(19) United States
(12) Patent Application Publication
(10) Pub. No.: US 2026/0066079 A1
(43) Pub. Date: Mar. 5, 2026

(54) METHODS, SYSTEMS, AND KITS FOR TREATMENT OF INFLAMMATORY DISEASES TARGETING TL1A

(71) Applicant: Prometheus Biosciences, Inc., San Diego, CA (US)

(72) Inventors: Fadi Towfic, La Jolla, CA (US); Yong Wang, San Diego, CA (US); Olivier Laurent, San Diego, CA (US); Jiandong Lu, Lexington, MA (US); Allison Luo, Rancho Santa Fe, CA (US)

(73) Assignee: Prometheus Biosciences, Inc., San Diego, CA (US)

(21) Appl. No.: 19/129,758

(22) PCT Filed: Nov. 20, 2023
(86) PCT No.: PCT/US2023/080443

§ 371 Date: May 14, 2025

Related U.S. Application Data
(60) Provisional application No. 63/384,925, filed on Nov. 23, 2022.
(60) Provisional application No. 63/385,622, filed on Nov. 30, 2022.
(60) Provisional application No. 63/478,865, filed on Jan. 6, 2023.
(60) Provisional application No. 63/487,853, filed on Mar. 1, 2023.

(51) Int. Cl. G16H 20/17; A61K 39/00; A61P 1/00; A61P 29/00; C07K 16/24; C07K 16/28; C12Q 1/6806; C12Q 1/6827; G16B 20/20; G16B 30/00; G16H 50/70

(52) U.S. Cl. CPC G16H 20/17; A61P 1/00; A61P 29/00; C07K 16/241; C07K 16/244; C07K 16/2842; C12Q 1/6806; C12Q 1/6827; G16B 20/20; G16B 30/00; G16H 50/70; A61K 2039/505; C07K 2317/52; C07K 2317/565; C07K 2317/567; C07K 2317/732; C07K 2317/734; C07K 2317/76; C12Q 2600/156

(57) ABSTRACT

Provided are methods, systems, and kits for selecting a subject for treatment with an inhibitor of Tumor necrosis factor-like cytokine 1A (TL1A) activity or expression based on a presence of one or more genotypes associated with a positive therapeutic response to the inhibitor of TL1A. Also provided are methods, systems and kits for detecting the one or more genotypes described herein.

### CROSS-REFERENCE TO RELATED APPLICATIONS

This application claims priority to U.S. Provisional Application No. 63/384,925 filed Nov. 23, 2022, U.S. Provisional Application No. 63/385,622 filed Nov. 30, 2022, U.S. Provisional Application No. 63/478,865 filed Jan. 6, 2023, and U.S. Provisional Application No. 63/487,853 filed Mar. 1, 2023.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Nov. 13, 2023, is named 25747-WO-PCT_SL.xml and is 1,647,264 bytes in size.

### 1. BACKGROUND

Inflammatory disease, fibrostenotic disease, and fibrotic disease pose a significant health burden worldwide due to the vast number of individuals affected and heterogeneous disease pathogenesis and varied clinical manifestations. One such disease is inflammatory bowel disease (IBD), which has two common forms, Crohn's disease (CD) and ulcerative colitis (UC). IBD is the chronic, relapsing inflammatory disorders of the gastrointestinal tract. Incidences of IBD are prevalent, affecting nearly three million individuals in the United States alone.

Few treatment options are available to patients that suffer from inflammatory disease, fibrostenotic disease, and fibrotic disease. Existing anti-inflammatory therapy such as steroids and tumor necrosis factor (TNF) inhibitors are typically used as a first line treatment for treating IBD. Unfortunately, a significant number of patients experience a lack of response or a loss of response to existing anti-inflammatory therapies, especially TNF inhibitors. While the patient is treated with an anti-inflammatory therapy that is ineffective, the disease worsens. Surgery, in the form of structureplasty (reshaping of the intestine) or resection (removal of the intestine), is the only treatment option for patients that do not respond to first line therapies. Surgical treatments for IBD are invasive, causing post-operative risks for an estimated third of patients undergoing surgery, such as anastomotic leak, infection, and bleeding.

The pathogenesis of inflammatory disease, fibrostenotic disease, and fibrotic disease, like IBD, is thought to involve an uncontrolled immune response that may be triggered by certain environmental factors in a genetically susceptible individual. The heterogeneity of disease pathogenesis and clinical course, combined with the variable response to treatment and its associated side effects, suggests a personalized medicine approach to treating these diseases is the best treatment strategy. Yet there are very few personalized therapies available to patients. Accordingly, there is a need to identify targeted therapeutic approaches f or the treatment of inflammatory disease, fibrostenotic disease, and fibrotic disease and subclinical phenotypes thereof, and an even greater need to develop reliable methodology to identifying patients who, based on their genotype, may respond to any given therapeutic approach. The needed methodologies would also identify subjects not yet diagnosed who are at risk of developing the disease, for which preventative interventions could be prescribed to reduce the growing health burden.

### 2. SUMMARY

The models and genotypes described herein are associated (individually or together) with (i) an increase in a level of TNFSF15 (TL1A) protein expression in a sample obtained from a subject or patient, as compared to a reference level of TNFSF15 (TL1A) protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. More specifically, models and genotypes described herein are associated (individually or together) with (i) alone; (ii) alone; (iii) alone; (iv) alone; (i) and (ii) together; (i) and (iii) together; (i) and (iv) together; (ii) and (iii) together; (ii) and (iv) together; (iii) and (iv) together; (i), (ii) and (iii) together; (i), (ii), and (iv) together; (i), (iii), and (iv) together; (ii), (iii), and (iv) together; or (i), (ii), (iii), and (iv) together, wherein (i), (ii), (iii), and (iv) corresponds to the (i), (ii), (iii), and (iv) numbered items in the previous clause of this paragraph. Accordingly and as an example, the models and genotypes described herein are associated (individually or together) with an increase in a level of TNFSF15 (TL1A) protein expression in a sample obtained from a subject or patient, as compared to a reference level of TNFSF15 (TL1A) protein expression (e.g., derived from a normal individual). Additionally, the models and genotypes described herein are associated (individually or together) with an increase of IBD enriched cell types in an IBD-affected tissue as compared to a reference level in a tissue not affected by IBD. Alternatively, the models and genotypes described herein are associated (individually or together) with a decrease of IBD depleted cell types in an IBD-affected tissue as compared to a reference level in a tissue not affected by IBD. Additionally, the models and genotypes described herein are associated (individually or together) with an increase of a positive therapeutic response in IBD patients to a treatment with the inhibitor of TL1A activity or expression, as compared to the reference level of response in patients not selected by the genotypes or models. The models provided herein, e.g. the models for calculating a Predictive Response Index (PRI), uses the genotypes described herein and applies a mathematical function to the genotypes to generate the PRI, as described in Sections 5.2 and 7. The genotypes disclosed herein are located at gene or genetic loci that are involved either directly or indirectly with TL1A-mediated or T-cell dependent inflammatory pathways. In addition, some of the genotypes provided herein are also significantly associated with inflammatory bowel disease (IBD), such as Crohn's disease (CD). The genotypes are useful for selecting a patient or a subject for treatment with an inhibitor of TL1A activity or expression. The patient may be diagnosed with IBD, CD, or both. The subject may be suspected of having IBD, CD, or both.

In one aspect, provided herein is a method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a Predictive Response Index (PRI) above a cutoff, wherein the PRI is calculated from a combination of polymorphisms determined from a sample from the subject and the PRI above the cutoff predicts a positive therapeutic response in the subject to a treatment with the inhibitor of TL1A activity or expression with a positive predictive value of at least about 29%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI above a cutoff indicates the subject is suitable for treatment with an inhibitor of TL1A activity or expression.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is above a cutoff.

In some embodiments, the method further comprises preparing DNA from the sample.

In one aspect, provided herein is a computer-implemented method of determining a Response Probability Score (RPS) for a subject, the method comprising:
(a) receiving genotype data obtained from a sample from the subject with an inflammatory, a fibrotic, or a fibrostenotic disease or condition, wherein the genotype data comprises a combination of polymorphisms;
(b) analyzing the genotype data with a first statistical algorithm configured to produce a Model Risk Score (MRS) for the subject by performing operations comprising:
   (i) assigning a weighted numerical value to each polymorphism in the combination of polymorphisms to produce a plurality of weighted values; and
   (ii) summing the plurality of weighted values;
(c) providing the MRS to a second statistical algorithm configured to perform a logarithmic function on the MRS to produce a Response Probability Score (RPS); and
(d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition.

In another aspect, provided herein is a computer-implemented method of determining a Response Probability Score (RPS) for a subject, the method comprising:
(a) obtaining a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
(b) receiving genotype data for a plurality of polymorphisms obtained from a sample from the subject;
(c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
(d) applying a logarithmic scale and a cutoff to the MRS to produce a Response Probability Score (RPS).

In some embodiments, the PRI is a Response Probability Score (RPS). In some embodiments, the PRI has a positive correlation coefficient with RPS. In some embodiments, the correlation coefficient between the PRI and RPS is Pearson correlation coefficient or Spearman correlation coefficient. In some embodiments, the positive correlation coefficient between the PRI and RPS is at least about 0.6, at least about 0.65, at least about 0.7, at least about 0.75, at least about 0.8, at least about 0.85, at least about 0.95, at least about 0.99, or 1. In some embodiments, the RPS ranges from 0 to 1. In some embodiments, the cutoff is 0.5. In some embodiments, the RPS is calculated as 1/(1+ e^{(-MRS)}), wherein the MRS is calculated as

$${β}_{0} + {\sum }_{i = 1}^{n} ( {β}_{i} ) \times ( {χ}_{i} ) ,$$

and wherein χᵢ is the mathematical representation of the ith single nucleotide polymorphisms (SNP) in the model and βᵢ is the weight for the ith SNP in the model.

In some embodiments, PRI is a Model Risk Score (MRS). In some embodiments, the PRI has a positive correlation coefficient with MRS. In some embodiments, the correlation coefficient between the PRI and MRS is Pearson correlation coefficient or Spearman correlation coefficient. In some embodiments, the positive correlation coefficient between the PRI and MRS is at least about 0.6, at least about 0.65, at least about 0.7, at least about 0.75, at least about 0.8, at least about 0.85, at least about 0.95, at least about 0.99, or 1. In some embodiments, the MRS is calculated as

$${β}_{0} + {\sum }_{i = 1}^{n} ( {β}_{i} ) \times ( {χ}_{i} ) ,$$

and wherein χᵢ is the mathematical representation of the ith SNP in the model.

In some embodiments, the SNP in the model is mathematically represented by χᵢ as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and 1 for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

In some embodiments, the combination of polymorphisms comprises one or more polymorphisms selected from Table 27, or a proxy polymorphism in linkage disequilibrium therewith as determined with an R² of at least 0.85, or a combination thereof.

In some embodiments, the cutoff is such that the PRI above the cutoff is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%. In some embodiments, the cutoff is such that the PRI above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%. In some embodiments, the cutoff is such that the PRI above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%. In some embodiments, the cutoff is such that the PRI above the cutoff is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%. In some embodiments, the cutoff is such that the PRI above the cutoff is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%. In some embodiments, the cutoff is such that the PRI above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%. In some embodiments, the cutoff is such that the PRI above the cutoff is predictive of an increase of one or more IBD enriched cell types with a positive predictive value of at least about 29%, 30%, 35%, 40%, 45%, 50%, 51%, 55%, 60%, 65% 70%, 75% 80%, 85%, 90% or 95%. In some embodiments, the cutoff is such that the PRI above the cutoff is predictive of a decrease of one or more IBD depleted cell types with a positive predictive value of at least about 29%, 30%, 35%, 40%, 45%, 50%, 51%, 55%, 60%, 65%, 70%, 75%, 80%, 85%, 90%, or 95%. In some embodiments, the cutoff is such that the PRI above the cutoff is predictive of an increase of one or more IBD enriched cell types with a specificity of at least about 50%, 55%, 60%, 65%, 70%, 75%, 80%, 85%, 90%, or 95%. In some embodiments, the cutoff is such that the PRI above the cutoff is predictive of a decrease of one or more IBD depleted cell types with a specificity of at least about 50%, 55%, 60%, 65%, 70%, 75%, 80%, 85%, 90%, or 95%. In some embodiments, the cutoff is such that the PRI above the cutoff is predictive of an increase of one or more IBD enriched cell types with a negative predictive value of at least about 50%, 55%, 60%, 65%, 70%, 75%, 80%, 85%, 90%, or 95%. In some embodiments, the cutoff is such that the PRI above the cutoff is predictive of a decrease of one or more IBD depleted cell types with a negative predictive value of at least about 50%, 55%, 60%, 65%, 70%, 75%, 80%, 85%, 90%, or 95%. In some embodiments, the cutoff is such that the PRI above the cutoff is predictive of an increase of one or more IBD enriched cell types with a sensitivity of at least about 20%, 25%, 30%, 35%, 40%, 45%, 50%, 55%, 60%, 65%, 70%, 75%, 80%, 85%, 90%, or 95%. In some embodiments, the cutoff is such that the PRI above the cutoff is predictive of a decrease of one or more IBD depleted cell types with a sensitivity of at least about 20%, 25%, 30%, 35%, 40%, 45%, 50%, 55%, 60%, 65%, 70%, 75%, 80%, 85%, 90%, or 95%. In some embodiments, the cutoff is such that the PRI above the cutoff is predictive of an increase of one or more IBD enriched cell types with a positive rate of at least about 10%, 15%, 20%, 25%, 30%, 35%, 40%, 45%, 50%, 55%, 60%, 65%, 70%, or 75%. In some embodiments, the cutoff is such that the PRI above the cutoff is predictive of a decrease of one or more IBD depleted cell types with a positive rate of at least about 10%, 15%, 20%, 25%, 30%, 35%, 40%, 45%, 50%, 55%, 60%, 65%, 70%, or 75%. In some embodiments, the cutoff is such that the PRI above the cutoff is predictive of an increase of one or more IBD enriched cell types with an accuracy of at least about 50%, 55%, 60%, 65%, 70%, 75%, 80%, 85%, 90%, or 95%. In some embodiments, the cutoff is such that the PRI above the cutoff is predictive of a decrease of one or more IBD depleted cell types with an accuracy of at least about 50%, 55%, 60%, 65%, 70%, 75%, 80%, 85%, 90%, or 95%.

In some embodiments, the one or more IBD enriched cell types comprises 1, 2, 3, 4, 5, 6, 7, 8, 9, 10, 11, 12, 13, or 14 cell types selected from the group consisting of activated fibroblasts, monocyte-derived dendritic cells (moDCs), and CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts.

In some embodiments, the one or more IBD depleted cell types comprises 1 or 2 cell types selected from the group consisting of Tuft cells and BEST4+ epithelial cells.

In some embodiments, the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

In some embodiments, the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

In one aspect, provided herein is a method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a comparison of a Predictive Response Index (PRI) to a cutoff according to (1) or (2):
(1) if the PRI has a positive correlation with a Response Probability Score (RPS), then the subject is selected if the PRI is above the cutoff; or
(2) if the PRI has a negative correlation with a RPS, then the subject is selected if the PRI is below the cutoff,
and wherein the PRI is calculated from a combination of polymorphisms determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 29%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2):
      (1) if the PRI has a positive correlation with RPS, then the subject is determined to be suitable if the PRI is above the cutoff; or
      (2) if the PRI has a negative correlation with RPS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the comparison is determined according to (1) or (2):
   (1) if the PRI has a positive correlation with RPS, then the subject is determined to be suitable for treatment with an inhibitor of TL1A activity or expression if the PRI is above the cutoff; or
   (2) if the PRI has a negative correlation with RPS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression based on a comparison of the PRI to a cutoff according to (1) or (2):
   (1) if the PRI has a positive correlation with RPS, then selecting the subject if the PRI is above the cutoff; or
   (2) if the PRI has a negative correlation with RPS, then selecting the subject if the PRI is below the cutoff.

In some embodiments, the method further comprises preparing DNA from the sample.

In one aspect, provided herein is a method of treating an inflammatory, a fibrotic, or a fibrostenotic disease or condition in a subject, the method comprising: administering to the subject a therapeutically effective amount of an inhibitor of TL1A activity or expression, based, at least partially, on a Predictive Response Index (PRI) calculated by applying one or more statistical algorithms to a combination of polymorphisms detected from a sample obtained from the subject and determining a comparison of the PRI to a cutoff to predicts a positive therapeutic response in the subject to a treatment with the inhibitor of TL1A activity or expression.

In another aspect, provided herein is a method of treating an inflammatory, a fibrotic, or a fibrostenotic disease or condition in a subject, the method comprising: administering an inhibitor of TL1A activity or expression to the subject that is predicted to exhibit a positive therapeutic response to the inhibitor of TL1A activity or expression, as determined by a Predictive Response Index (PRI) that is calculated by:
(a) detecting a presence of a combination of polymorphisms in a sample from the subject;
(b) applying a statistical algorithm to the combination of polymorphisms detected in step (a) to generate the PRI; and
(c) determining a comparison of the PRI to a cutoff.

In a further aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the PRI is further determined in a comparison to a cutoff; and
(b) treating the subject by administering a therapeutically effective amount of the inhibitor of TL1A activity or expression to the subject.

In some embodiments, the method further comprises preparing DNA from the sample.

In some embodiments, the comparison of PRI to a cutoff is determined according to (1) or (2): (1) if the PRI has a positive correlation with RPS, then determining the PRI of the subject if the PRI is above the cutoff, or (2) if the PRI has a negative correlation with RPS, then determining the PRI of the subject if the PRI is below the cutoff.

In some embodiments, the correlation coefficient is Pearson correlation coefficient or Spearman correlation coefficient.

In some embodiments, if the PRI has a positive correlation with RPS then the positive correlation coefficient is at least about 0.6, at least about 0.65, at least about 0.7, at least about 0.75, at least about 0.8, at least about 0.85, at least about 0.95, at least about 0.99, or 1, or if the PRI has a negative correlation with RPS then the negative correlation coefficient is at most about −0.6, at most about −0.65, at most about −0.7, at most about −0.75, at most about −0.8, at most about −0.85, at most about −0.95, at most about −0.99, or −1.

In some embodiments, the RPS ranges from 0 to 1.

In some embodiments, if the PRI has a positive correlation with RPS then the cutoff is 0.5, or if the PRI has a negative correlation with RPS then the cutoff is −0.5.

In some embodiments, the RPS is calculated as 1/(1+e(−MRS)), wherein the MRS is calculated as

$$β0 + {\sum }_{i = 1}^{n} ( {β}_{i} ) \times ( {χ}_{i} ) ,$$

and wherein χᵢ is the mathematical representation of the ith single nucleotide polymorphisms (SNP) in the model and βi is the weight for the ith SNP in the model.

In some embodiments, the SNP in the model is mathematically represented by χᵢ as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and 1 for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

In some embodiments, the combination of polymorphisms comprises one or more polymorphisms selected from Table 27, or a proxy polymorphism in linkage disequilibrium therewith as determined with an R2 of at least 0.85, or a combination thereof.

In some embodiments, the cutoff is such that the comparison of the PRI to the cutoff according to (1) or (2) is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%. In some embodiments, the cutoff is such that the comparison of the PRI to the cutoff according to (1) or (2) is predictive of a positive therapeutic response in the subject to a treatment with the inhibitor of TL1A activity or expression with a specificity of at least about 50%, 55%, 60%, 65%, 70%, 75%, 80%, 85%, 90%, or 95%. In some embodiments, the cutoff is such that the comparison of the PRI to the cutoff according to (1) or (2) is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%. In some embodiments, the cutoff is such that the comparison of the PRI to the cutoff according to (1) or (2) is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85% 90%, or 95%. In some embodiments, the cutoff is such that the comparison of the PRI to the cutoff according to (1) or (2) is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%. In some embodiments, the cutoff is such that the comparison of the PRI to the cutoff according to (1) or (2) is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%. In some embodiments, the cutoff is such that the comparison of the PRI to the cutoff according to (1) or (2) is predictive of an increase of one or more IBD enriched cell types with a positive predictive value of at least about 29%, 30%, 35%, 40%, 45%, 50%, 51%, 55%, 60%, 65%, 70%, 75%, 80%, 85%, 90%, or 95%. In some embodiments, the cutoff is such that the comparison of the PRI to the cutoff according to (1) or (2) is predictive of a decrease of one or more IBD depleted cell types with a positive predictive value of at least about 29%, 30%, 35%, 40%, 45%, 50%, 51%, 55%, 60%, 65%, 70%, 75%, 80%, 85%, 90%, or 95%. In some embodiments, the cutoff is such that the comparison of the PRI to the cutoff according to (1) or (2) is predictive of an increase of one or more IBD enriched cell types with a specificity of at least about 50%, 55%, 60%, 65%, 70%, 75%, 80%, 85%, 90%, or 95%. In some embodiments, the cutoff is such that the comparison of the PRI to the cutoff according to (1) or (2) is predictive of a decrease of one or more IBD depleted cell types with a specificity of at least about 50%, 55%, 60%, 65%, 70%, 75%, 80%, 85%, 90%, or 95%. In some embodiments, the cutoff is such that the comparison of the PRI to the cutoff according to (1) or (2) is predictive of an increase of one or more IBD enriched cell types with a negative predictive value of at least about 50%, 55%, 60%, 65%, 70%, 75%, 80%, 85%, 90%, or 95%. In some embodiments, the cutoff is such that the comparison of the PRI to the cutoff according to (1) or (2) is predictive of a decrease of one or more IBD depleted cell types with a negative predictive value of at least about 50%, 55%, 60%, 65%, 70%, 75%, 80%, 85%, 90%, or 95%. In some embodiments, the cutoff is such that the comparison of the PRI to the cutoff according to (1) or (2) is predictive of an increase of one or more IBD enriched cell types with a sensitivity of at least about 20%, 25%, 30%, 35%, 40%, 45%, 50%, 55%, 60%, 65%, 70%, 75%, 80%, 85%, 90%, or 95%. In some embodiments, the cutoff is such that the comparison of the PRI to the cutoff according to (1) or (2) is predictive of a decrease of one or more IBD depleted cell types with a sensitivity of at least about 20%, 25%, 30%, 35%, 40%, 45%, 50%, 55%, 60%, 65%, 70%, 75%, 80%, 85%, 90%, or 95%. In some embodiments, the cutoff is such that the comparison of the PRI to the cutoff according to (1) or (2) is predictive of an increase of one or more IBD enriched cell types with a positive rate of at least about 10%, 15%, 20%, 25% 30%, 35%, 40%, 45%, 50%, 55%, 60%, 65%, 70%, or 75%. In some embodiments, the cutoff is such that the comparison of the PRI to the cutoff according to (1) or (2) is predictive of a decrease of one or more IBD depleted cell types with a positive rate of at least about 10%, 15%, 20%, 25%, 30%, 35%, 40%, 45%, 50%, 55%, 60%, 65%, 70%, or 75%. In some embodiments, the cutoff is such that the comparison of the PRI to the cutoff according to (1) or (2) is predictive of an increase of one or more IBD enriched cell types with an accuracy of at least about 50%, 55%, 60%, 65%, 70%, 75%, 80%, 85%, 90%, or 95%. In some embodiments, the cutoff is such that the comparison of the PRI to the cutoff according to (1) or (2) is predictive of a decrease of one or more IBD depleted cell types with an accuracy of at least about 50%, 55%, 60%, 65%, 70%, 75%, 80%, 85%, 90%, or 95%. In some embodiments, the (1) and (2) referenced in this paragraph are the (1) and (2) clauses described in this Section (Section 2), e.g. in the applicable preceding paragraphs.

In some embodiments, the one or more IBD enriched cell types comprises 1, 2, 3, 4, 5, 6, 7, 8, 9, 10, 11, 12, 13, or 14 cell types selected from the group consisting of activated fibroblasts, monocyte-derived dendritic cells (moDCs), and CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts.

In some embodiments, the one or more IBD depleted cell types comprises 1 or 2 cell types selected from the group consisting of Tuft cells and BEST4+ epithelial cells.

In some embodiments, the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

In some embodiments, the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

In some embodiments, (i) the PRI is calculated with a combination of polymorphisms selected from the combinations listed in column 2 of Table 31 and corresponding β coefficients listed in column 1 of Table 31; (ii) the MRS is calculated with a combination of polymorphisms selected from the combinations listed in column 2 of Table 31 and corresponding β coefficients listed in column 1 of Table 31; and/or (iii) the RPS is calculated with a combination of polymorphisms selected from the combinations listed in column 2 of Table 31 and corresponding β coefficients listed in column 1 of Table 31.

In some embodiments, the combination of polymorphisms is detected in the sample by subjecting the sample to an assay configured to detect a presence of at least three nucleotides corresponding to nucleic acid position 501 within at least three of SEQ ID NOS: 2001-2048 and 2057-2059.

In one aspect, provided herein is a computer-implemented system comprising at least one processor and instructions executable by the at least one processor to provide an application configured to determine a Response Probability Score (RPS) for a subject by performing operations comprising:
(a) receiving genotype data obtained from a sample from the subject with an inflammatory, a fibrotic, or a fibrostenotic disease or condition, wherein the genotype data comprises a combination of polymorphisms;
(b) applying a first statistical algorithm to the genotype data, the first statistical algorithm configured to produce a Model Risk Score (MRS) for the subject by performing operations comprising:
   (i) assigning a weighted numerical value to each polymorphism in the combination of polymorphisms to produce a plurality of weighted values; and
   (ii) summing the plurality of weighted values;
(c) applying a second statistical algorithm to the MRS, the second statistical algorithm configured to perform a logarithmic function on the MRS to produce a Response Probability Score (RPS); and
(d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition

In another aspect, provided herein is a computer-implemented system comprising at least one processor and instructions executable by the at least one processor to provide an application configured to determine a Response Probability Score (RPS) for a subject by performing operations comprising:
(a) receiving a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
(b) receiving genotype data for a plurality of polymorphisms obtained from a sample from the subject;
(c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
(d) applying a logarithmic scale and a cutoff to the MRS to produce a Response Probability Score (RPS).

In some embodiments of the computer-implemented system, the RPS ranges from 0 to 1.

In some embodiments of the computer-implemented system, the cutoff is 0.5.

In some embodiments of the computer-implemented system, the genotype data is a combination of single nucleotide polymorphisms (SNPs).

In some embodiments of the computer-implemented system, the RPS is calculated as 1/(1+e^{(-MRS)}), wherein the MRS is calculated as

$${β}_{0} + {\sum }_{i = 1}^{n} ( {β}_{i} ) \times ( {χ}_{i} )$$

and wherein χᵢ is the mathematical representation of the ith single nucleotide polymorphisms (SNP) in the model and βᵢ is the weight for the ith SNP in the model.

In some embodiments of the computer-implemented system, the MRS is calculated as

$${β}_{0} + {\sum }_{i = 1}^{n} ( {β}_{i} ) \times ( {χ}_{i} ) ,$$

and wherein χᵢ is the mathematical representation of the ith SNP in the model.

In some embodiments of the computer-implemented system, the SNP in the model is mathematically represented by χᵢ as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and 1 for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

In some embodiments of the computer-implemented system, the combination of polymorphisms comprises one or more polymorphisms selected from Table 27, or a proxy polymorphism in linkage disequilibrium therewith as determined with an R² of at least 0.85, or a combination thereof.

In some embodiments of the computer-implemented system, the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

In some embodiments of the computer-implemented system, the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

In some embodiments of the computer-implemented system, (i) the MRS is calculated with a combination of polymorphisms selected from the combinations listed in column 2 of Table 31 and corresponding β coefficients listed in column 1 of Table 31; and/or (ii) the RPS is calculated with a combination of polymorphisms selected from the combinations listed in column 2 of Table 31 and corresponding β coefficients listed in column 1 of Table 31.

In some embodiments of the computer-implemented system, the combination of polymorphisms is detected in the sample by subjecting the sample to an assay configured to detect a presence of at least three nucleotides corresponding to nucleic acid position 501 within at least three of SEQ ID NOS: 2001-2048 and 2057-2059.

In some embodiments of the methods or the computer-implemented systems provided herein, the subject has been treated with an advanced IBD therapy prior to the treatment with the inhibitor of TL1A activity or expression. In some embodiments of the methods or the computer-implemented systems provided herein, the subject has not been treated with an advanced IBD therapy prior to the treatment with the inhibitor of TL1A activity or expression. In some embodiments of the methods or the computer-implemented systems provided herein, including in this paragraph, the advanced IBD therapy comprises one or more selected from the group consisting of a biologic therapeutic agent for IBD, an S1P1 modulator, or a JAK inhibitor. In some embodiments of the methods or the computer-implemented systems provided herein, including in this paragraph, the biologic therapeutic agent for IBD comprises an anti-TNFα antibody, an anti-IL23 antibody, or an anti-integrin antibody. In some embodiments of the methods or the computer-implemented systems provided herein, including in this paragraph, the anti-TNFα comprises adalimumab, infliximab, golimumab, certolizumab, or etanercept. In some embodiments of the methods or the computer-implemented systems provided herein, including in this paragraph, the anti-IL23 antibody comprises ustekinumab, guselkumab, risankizumab, brazikumab, mirikizumab, tildrakizumab, or briakinumab. In some embodiments of the methods or the computer-implemented systems provided herein, including in this paragraph, the anti-integrin antibody comprises etrolizumab, vedolizumab, natalizumab, or ontamalimab. In some embodiments of the methods or the computer-implemented systems provided herein, including in this paragraph, the S1P1 modulator comprises fingolimod, siponimod, etrasimod, ozanimod, ponesimod, amiselimod, ceralifimod, or mocravimod. In some embodiments of the methods or the computer-implemented systems provided herein, including in this paragraph, the JAK inhibitor comprises tofacitinib, abrocitinib, baricitinib, upadacitinib, or filgotinib.

In some embodiments, the inhibitor of TL1A activity or expression is an antibody or antigen binding fragment thereof that binds to TL1A (anti-TL1A antibody or antigen binding fragment), wherein the anti-TL1A antibody or antigen binding fragment comprises a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5, and (c) an HCDR3 comprising an amino acid sequence set forth by any one of SEQ ID NOS: 6-9; and a light chain variable region comprising: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15.

In some embodiments, the inhibitor of TL1A activity or expression is an anti-TL1A antibody or antigen binding fragment, wherein the anti-TL1A antibody or antigen binding fragment comprises a heavy chain variable domain comprising an amino acid sequence at least about 90% identical to any one of SEQ ID NOS: 101-135, or 310-302, and a light chain variable domain comprising an amino acid sequence at least about 90% identical to any one of SEQ ID NOS: 201-206 or 303. In some embodiments, the heavy chain variable domain comprises an amino acid sequence at least about 91%, 92%, 93%, 94%, 95%, 96%, 97%, 98%, 99%, or 100% identical to any one of SEQ ID NOS: 101-135, or 310-302. In some embodiments, the light chain variable domain comprises an amino acid sequence at least about 91%, 92%, 93%, 94%, 95%, 96%, 97%, 98%, 99%, or 100% identical to any one of SEQ ID NOS: 201-206 or 303.

In some embodiments, the inhibitor of TL1A activity or expression is an anti-TL1A antibody or antigen binding fragment, wherein the anti-TL1A antibody or antigen binding fragment comprises: (a) a heavy chain variable framework region comprising a human IGHV1-46*02 framework or a modified human IGHV1-46*02 framework; and (b) a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework. In some embodiments, an amino acid modification of the less than 14 amino acid modifications comprises: (a) the amino acid modification is at position 47 in the heavy chain variable region, and the amino acid at position 47 is R, N, D, C, Q, E, G, H, I, L, K, M, F, P, S, T, W, Y, or V; (b) the amino acid modification is at position 45 in the heavy chain variable region, and the amino acid at position 45 is A, N, D, C, Q, E, G, H, I, L, K, M, F, P, S, T, W, Y, or V; (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N, D, C, Q, E, G, H, I, L, K, M, F, P, S, T, W, or Y; (e) the amino acid modification is at position 80 in the heavy chain variable region, and the amino acid at position 80 is A, R, N, D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V; (g) the amino acid modification is at position 89 in the heavy chain variable region, and the amino acid at position 89 is A, R, N, D, C, Q, E, G, H, I, L, K, M, F, P, S, T, W, or Y; or (h) the amino acid modification is at position 91 in the heavy chain variable region, and the amino acid at position 91 is A, R, N, D, C, Q, E, G, H, I, L, K, F, P, S, T, W, Y, or V; or a combination of two or more modifications selected from (a) to (h). In some embodiments, an amino acid modification of the less than 14 amino acid modifications comprises: A47R, R45K, M55I, V78A, M80I, R82T, V89A, M91L in the heavy chain variable region, per Aho or Kabat numbering. In some embodiments, an amino acid modification of the less than 14 amino acid modifications comprises: (a) a modification at amino acid position 54 in the light chain variable region; and/or (b) a modification at amino acid position 55 in the light chain variable region; per Aho or Kabat numbering. In some embodiments, an amino acid modification of the less than 14 amino acid modifications comprises: (a) the amino acid modification is at position 54 of the light chain variable region, and the amino acid at position 54 is A, R, N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V. In some embodiments, an amino acid modification of the less than 14 amino acid modifications comprises L54P and/or L55 W in the light chain variable region, per Aho or Kabat numbering.

In some embodiments, the inhibitor of TL1A activity or expression is an antibody or antigen binding fragment thereof that binds to TL1A and comprises: a heavy chain variable region comprising SEQ ID NO: 301 X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V. In some embodiments, the inhibitor of TL1A activity or expression is an antibody or antigen binding fragment thereof that binds to TL1A and comprises: a heavy chain variable region comprising SEQ ID NO: 302 X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYC[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V. In some embodiments, (a) X1 is Q or E, (b) X2 is R or K (c) X3 is A or R; (d) X4 is M or I; (e) X5 is V or A; (f) X6 is M or I; (g) X7 is R or T; (h) X8 is V or A; (i) X9 is M or L (j) X10 is L or P; (k) X11 is L or W; or (l) X1-X11 are any combination of (a) to (k).

In some embodiments, the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15.

In some embodiments, the antibody or antigen binding fragment comprises a heavy chain framework (FR) 1 as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof.

In some embodiments, the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG16, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of two or more selected from (a)-(uu), per Kabat numbering.

In some embodiments, the antibody or antigen binding fragment comprises a human IgG4 Fc region. In some embodiments, the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362. In some embodiments, the antibody of antigen binding fragment comprises a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1. In some embodiments, the antibody or antigen binding fragment comprises a Fc region and wherein the Fc comprises the human IgG1 comprises SEQ ID NO: 320. In some embodiments, the antibody or antigen binding fragment comprises a Fc region and wherein the ADCC function of the Fc region comprising reduced ADCC is at least about 50% reduced as compared to human IgG1. In some embodiments, the antibody or antigen binding fragment comprises a Fc region and wherein the CDC function of the Fc region comprising reduced CDC is at least about 50% reduced as compared to human IgG1. In some embodiments, the antibody or antigen binding fragment comprises a Fc region and wherein the Fc comprises (i) a human IgG4 Fc region or (ii) a human IgG4 Fc region comprising (a) S228P, (b) S228P and L235E, or (c) S228P, F234A, and L235A, per Kabat numbering. In some embodiments, the antibody or antigen binding fragment comprises a Fc region and wherein the Fc comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). In some embodiments, the antibody or antigen binding fragment comprises a Fe region and wherein the Fc comprises a human IgG1 with a substitution selected from 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering.

In some embodiments, the antibody or antigen binding fragment comprises a Fc region and wherein the Fe comprises a sequence at least about 90%, 91%, 92%, 93%, 94%, 95%, 96%, 97%, 98%, 99%, or 100% identical to any one of SEQ ID NOS: 320-362. In some embodiments, the antibody or antigen binding fragment comprises a Fc region and wherein the Fe comprises any one of SEQ ID NOs: 401-413 or a sequence at least about 90%, 91%, 92%, 93%, 94%, 95%, 96%, 97%, 98%, or 99% identical to any one of SEQ ID NOs: 401-413. In some embodiments, the antibody or antigen binding fragment comprises a heavy chain comprising any one of SEQ ID NOs: 501-513 or a sequence at least about 90%, 91%, 92%, 93%, 94%, 95%, 96%, 97%, 98%, or 99% identical to any one of SEQ ID NOs: 501-513. In some embodiments, the antibody or antigen binding fragment comprises a light chain comprising any one of SEQ ID NO: 514 or a sequence at least about 90%, 91%, 92%, 93%, 94%, 95%, 96%, 97%, 98%, or 99% identical to any one of SEQ ID NO: 514.

In some embodiments, the combination of polymorphisms are detected in the sample by subjecting the sample to an assay configured to detect a presence of combination of nucleotides corresponding to nucleic acid position 501 within a combination of sequences selected from SEQ ID NOS: 2001-2041, and 2057-2059.

In some embodiments, the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis. In some embodiments, the Crohn's disease is ileal, ileocolonic, or colonic Crohn's disease. In some embodiments, the subject has, or is at risk for developing, a non-response or loss-of-response to a standard therapy comprising glucocorticosteroids, anti-TNF therapy, anti-a4-b7 therapy, anti-IL12p40 therapy, or a combination thereof.

In some embodiments, the method further comprises determining whether the subject with an inflammatory, a fibrotic, or a fibrostenotic disease or condition is suitable for treatment with an inhibitor of TL1A activity or expression based, at least in part, on the at least three polymorphisms detected in the sample. In some embodiments, the at least three polymorphisms are detected in utilizing assay comprising a quantitative polymerase chain reaction (qPCR), nucleic acid sequencing reaction, or a genotyping array.

In some embodiments, the combination of polymorphsims comprises or consists of any combination of polymorphisms described in row x of column 2 of Table 31, wherein x is any number between 2 to 1374.

In some embodiments, the combination of polymorphisms comprises or consists of any combination of polymorphisms described in row x of column 2 of Table 31, wherein x is any number between 2 to 1374, wherein the polymorphisms of the combination of polymorphisms have β coefficients described in the row x of column 1 of Table 1, and wherein the polymorphisms of the combination of polymorphisms are numerically encoded as described in the row x of column 2 of Table 1.

In further embodiments, at least three polymorphisms is at least eight polymorphisms. In some embodiments, the at least eight polymorphisms are provided in an 8-SNP model in Table 25.

In some embodiments, the method further comprises providing the sample to determine PRI, MRS, or RPS. In some embodiments, the method further comprises selecting the subject according to PRI, MRS, or RPS.

In some embodiments, the method further comprises: contacting genetic materials in the sample with one or more nucleic acid primer pairs having forward and reverse primers capable of hybridizing to one or more target nucleic acid sequences, the one or more target nucleic acid sequences collectively comprising chromosome positions of the polymorphisms of row x of column 2 of Table 31, wherein x is any number between 2 to 1374, amplifying the target nucleic acid sequences by polymerase chain reactions with the nucleic acid primer pairs of the contacting step, inputting results from the amplifying step into a computer system, and analyzing the results via the computer system to determine PRI, RPS, or MRS, wherein the computer system comprises a storage unit configured to store the parameters of row y of column 1 of Table 31, wherein the y is identical to the x in the contacting step.

In some embodiments, the β₀ used for calculating MRS is about 0.0077127943934849 or about 0.008. In some embodiments, the cutoff for MRS is about 0.0322446725024791 or about 0.03.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### 3. INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### 4. BRIEF DESCRIPTION OF THE DRAWINGS

The novel features of the inventive concepts set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a workflow according to an embodiment of the present disclosure for processing a biological sample obtained from a subject to inform the selection of a therapeutic agent to treat a disease or a condition of the subject.

FIG. 2 shows a computer-implemented workflow according to an embodiment of the present disclosure for generating an electronic report to a user, such as a physician, comprising a TNFSF15 profile of a subject based on an analysis of genotype data from the subject.

FIG. 3 shows a computer system that is programmed or otherwise configured to implement methods provided herein.

FIG. 4 shows a computer-implemented workflow according to an embodiment of the present disclosure for producing a TNFSF15 profile.

FIG. 5A-5C shows a clustering analysis within our the TL1A companion diagnostic (CDx) dataset according to some embodiments herein. FIG. 5A shows cluster 1, FIG. 5B shows cluster 2, and FIG. 5C shows cluster 3, from the TL1A CDx dataset.

FIG. 6 shows that the 3 clusters from FIG. 5A-5C were collapsed into 2 clusters (high TL1A expression clusters shown on the left) and (low TL1A expression clusters on the right).

FIGS. 7A-7C show chromatograms for analytical size exclusion chromatography of anti-TL1A antibodies. FIG. 7A shows chromatograms for analytical size exclusion chromatography of antibodies A193, A194, and A195. FIG. 7B shows chromatograms for analytical size exclusion chromatography of antibodies A196, A197, and A198. FIG. 7C shows chromatograms for analytical size exclusion chromatography of antibodies A199, A200, and A201.

FIG. 8A-8B depict inhibition of interferon gamma in human blood with anti-TL1A antibodies. FIG. 8A depicts inhibition of interferon gamma in human blood with anti-TL1A antibodies A219 and A213. FIG. 8B depicts inhibition of interferon gamma in human blood with anti-TL1A antibody A212.

FIGS. 9A-9C depicts a PLS model demonstrating effect of pH and protein concentration on viscosity. FIG. 9A shows a PLS graph, FIG. 9B shows a model of the predicted viscosity versus anti-TL1A antibody concentration in mg/mL, and FIG. 9C shows a model of the estimated viscosity versus actual viscosity. Viscosity units are in mPa-s.

FIG. 10 illustrates, according to some embodiments, the TL1A CDx performance statistics against TL1A ex vivo production assay.

FIGS. 11A-11C show classes or sub-classes of cells in gastrointestinal tissues are distinguishable based on gene expression profile established from single cell RNA sequencing. FIG. 11A shows that cell types and sub-cell types listed in the figure legend are distinguishable based on gene expression profile established from single cell RNA sequencing. FIG. 11B shows that B cells, epithelial cells, innate lymphoid cells (ILCs) cells, myloid cells, plasma cells, stromal cells, and T cells are distinguishable based on gene expression profile established from single cell RNA sequencing. FIG. 11C shows that various classes and subclasses of cells can also be identified by hierarchical clustering analyses.

FIGS. 11D-11K show that various cell types and sub-cell types from gastrointestinal tissues can be distinguished based on gene expression profile established from single cell RNA sequencing and validated with known cell type markers. FIG. 11D shows that B cells, epithelial cells, innate lymphoid cells (ILCs) cells, mast cells, MNPs, pDCs, plasma cells, stromal cells, and T cells are distinguishable based on gene expression profile established from single cell RNA sequencing and validated with the known gene expression markers for each cell type. In FIG. 11D, the cell types are listed on the top, the known cell markers are listed in the left axis, and each column in the figure represents one single cell. FIG. 11E shows the same results and data as FIG. 1D, except that averages across all single cells under each cell type are presented. FIG. 11F is based on the same data as FIG. 11D and FIG. 11E, except that unsupervised analyses of differential expressed genes (DEG) were performed and the top 10 DEGs for each cell type are shown. FIG. 11G shows that sub-cell types within the epithelial cells, including BEST+ epithelial cells, colonocytes, EECs, enterocytes, Goblet cells, microfold cells, Paneth cells, stem cells, TA cells, and Tuft cells are distinguishable based on gene expression profile established from single cell RNA sequencing and validated with the known gene expression markers for each cell type. In FIG. 11G, the cell types are listed on the top, the known cell markers are listed in the left axis, and each column in the figure represents one single cell. FIG. 11H shows the same results and data as FIG. 11G, except that averages across all single cells under each cell type are presented. FIG. 11I shows that sub-cell types within the endothelial cells, including ACKR1+ endothelial cells, activated fibroblasts, CD36+ endothelial cells, fibroblasts, glial cells, lymphatics, myofibroblasts, pericytes, and smooth muscle cells are distinguishable based on gene expression profile established from single cell RNA sequencing and validated with the known gene expression markers for each cell type. In FIG. 11I, the cell types are listed on the top, the known cell markers are listed in the left axis, and each column in the figure represents one single cell. FIG. 11J shows that sub-cell types within the T cells, including cytotoxic T cells, highly activated T cells, ILC1, ILC3, naïve/CM T cells, natural killer (NK) cells, NK T-cells, TFH-like cells, Gamma-delta (γδ) T cells, regulatory T cells, and tissue-resident memory T (Trm) cells are distinguishable based on gene expression profile established from single cell RNA sequencing and validated with the known cytokine markers for each cell type. In FIG. 11J, the cell types are listed at the bottom, the known cell markers are listed in the left axis, the size of the dot depicts the percent of the corresponding cell type expressing that cytokine, and the color of the dot depicts the level of expression based on RNA-seq, normalized so that the total reads are the same across the cells. FIG. 11K shows that sub-cell types within the myloid cells, including activated dendritic cells (DCs), DC1, DC2, monocyte-derived dendritic cells (moDC), plasmacytoid dendritic cells (pDCs), inflammatory macrophages, and resident macrophages are distinguishable based on gene expression profile established from single cell RNA sequencing and validated with the known gene expression markers for each cell type. In FIG. 11K, the cell types are listed on the top, the known cell markers are listed in the left axis, and each column in the figure represents one single cell. In FIGS. 11D-11I and 11K, gene expression levels are depicted by color-coded expression score, with 0 being the lowest and 6 being the highest, normalized so that the total reads are the same across the cells.

FIG. 12A shows the procedure for validating the MuSiC methodology of deconvoluting bulk RNA sequencing data. FIG. 12B right panel shows the single cells (X-axis at the bottom) used to construct the pseudo-bulk RNA sequencing data, FIG. 12B left panel shows the cell types (X-axis at the bottom) and their respective proportions (color of the heat map) deconvoluted from the pseudo-bulk RNA sequencing data, and the validation in FIG. 12B was performed for multiple patients as indicated in the Y-axis as subject numbers. FIG. 12C shows that the absolute difference between FIG. 12B left and right panel is small, indicating that the cell types and their respective proportions deconvoluted from the pseudo-bulk RNA sequencing data matched well with the single cell RNA-seq data used to construct the pseudo-bulk data. FIG. 12D right panel shows the single cells (X-axis at the bottom) used to construct the pseudo-bulk RNA sequencing data, FIG. 12D left panel shows the cell types (X-axis at the bottom) and their respective proportions (color of the heat map) deconvoluted from the pseudo-bulk RNA sequencing data, and the validation in FIG. 12D was performed for multiple patients as indicated in the Y-axis as subject numbers. FIG. 12E shows that the absolute difference between FIG. 12D left and right panels is small, indicating that the cell types and their respective proportions deconvoluted from the pseudo-bulk RNA sequencing data matched well with the single cell RNA-seq data used to construct the pseudo-bulk data. In FIG. 12B and FIG. 12D, the color of the heatmap indicates the proportion of the cells according to the color legend, and in FIG. 12C and FIG. 12E, the color of the difference map indicates the magnitude of difference according to the color legend.

FIG. 13A shows that 31 cell types or sub-cell types were identified in Mount Sinai GSE83687 RNAseq Dataset with enrichment or reduction in UC or CD patient tissues as compared control samples. FIG. 13B shows that 34 cell types or sub-cell types were identified in Lloyd Price GSE111889 RNAseq Dataset with enrichment or reduction in UC or CD patient tissues as compared control samples. FIG. 13C shows that 20 cell types or sub-cell types were identified in UC119 with enrichment or reduction in UC patient tissues. FIG. 13D shows that 20 cell types or sub-cell types were identified in CD100 with enrichment or reduction in CD patient tissues. FIG. 13E shows that the deconvoluted cell types and cell type proportions demonstrated enrichment of Paneth cells in small bowel and ileum over other tissues in the gastrointestinal tract, consistent with the known distribution of Paneth cells. FIG. 13F shows that deconvoluted cell types and cell type proportions demonstrated enrichment of Paneth cells in intestinal tissues over rectum or colon, consistent with the known distribution of Paneth cells.

FIGS. 13G-13J show that activated fibroblasts were enriched in both CD and UC diseased tissues as compared to non-diseased control tissues, based on deconvolution of Mount Sinai GSE83687 RNAseq Dataset (FIG. 13G), Lloyd Price GSE111889 RNAseq Dataset (FIG. 13H), UC119 (FIG. 13I), and CD100 (FIG. 13J). FIGS. 13K-13N show that monocyte derived dendritic cells (moDCs) were enriched in both CD and UC diseased tissues as compared to non-diseased control tissues, based on deconvolution of Mount Sinai GSE83687 RNAseq Dataset (FIG. 13K), Lloyd Price GSE111889 RNAseq Dataset (FIG. 13L), UC119 (FIG. 13M), and CD100 (FIG. 13N). FIGS. 13O-13R show that CD36+ endothelial cells were enriched in both CD and UC diseased tissues as compared to non-diseased control tissues, based on deconvolution of Mount Sinai GSE83687 RNAseq Dataset (FIG. 13O), Lloyd Price GSE111889 RNAseq Dataset (FIG. 13P), UC119 (FIG. 13Q), and CD100 (FIG. 13R). FIGS. 13S-13V show that BEST4+ enterocytes/epithelial cells were reduced in CD and/or UC diseased tissues as compared to non-diseased control tissues, based on deconvolution of Mount Sinai GSE83687 RNAseq Dataset (FIG. 13S), Lloyd Price GSE111889 RNAseq Dataset (FIG. 13T), UC119 (FIG. 13U), and CD100 (FIG. 13V). FIGS. 13W-13X show that Tuft cells were reduced in CD and/or UC diseased tissues as compared to non-diseased control tissues, based on deconvolution of Mount Sinai GSE83687 RNAseq Dataset (FIG. 13W) and Lloyd Price GSE111889 RNAseq Dataset (FIG. 13X). In FIGS. 13A-13X, the y-axis depicts the proportion of the identified cells in all cells.

FIG. 14 shows the software design architecture of the assay interpretation software, according to some embodiments herein.

FIGS. 15A-15K show screenshots of the assay interpretation software application from to illustrate the user experience, according to some embodiments herein. The assay interpretation software application comprises a user interface that comprises a login view (FIG. 15A), a files view (FIG. 15B), a samples view (FIG. 15C), a data analyses view (FIG. 15D), a new data analysis view (FIG. 15E), system settings view (FIG. 15F), a clinical settings view (FIG. 15G), a clinical study settings view (FIG. 15H), a user management view (FIG. 15I), an activities view (FIG. 15J), or a my profile view (FIG. 15K), or any combination thereof.

FIGS. 16A-16B show exemplary clinical trial studies of the anti-TL1A antibodies disclosed herein. FIG. 16A depicts the study schema for induction period for the phase 2 clinical trial for A219 in UC in accordance with some embodiments herein. FIG. 16B depicts the study schema for open-label extension period for the phase 2 clinical trial for A219 in UC in accordance with some embodiments herein.

FIGS. 17A-17I show comparisons of clinical remission and endoscopic improvement between CDx classified patients and all patients for various CDx models, wherein the CDx models were optimized to predict the enrichment in the IBD-affected tissue of ileum entero_clonocytes (FIG. 17A), colon moDC (FIG. 17B and FIG. 17C), colon goblet (FIG. 17D and FIG. 17E), colon resident macrophage (FIG. 17F and FIG. 17G), and colon transit-amplifying (TA) progenitor cells (FIG. 17H and FIG. 17I), as further described in Section 7.23 (Example 23).

FIG. 17J shows that CD patients that have been treated with an advanced IBD therapy have a robust response after A219 treatment, as measured by both clinical remission.

FIG. 17K shows that UC patients that have not been treated with an advanced IBD therapy (“Naïve” in FIG. 17K) have a higher response rate than both the patients that have already been treated with an advanced IBD therapy (“Experienced” in FIG. 17K) and the overall patient population (both naïve and experienced, shown as “Overall” in FIG. 17K), as measured by both clinical remission (left panel in FIG. 17K) and endoscopic improvement (right panel in FIG. 17K).

### 5. DETAILED DESCRIPTION

Provided herein are methods, systems, and kits for treating a subject who may be suitable for treatment with an inhibitor of Tumor Necrosis Factor (Ligand) Superfamily, Member 15 (TL1A) activity or expression, provided the subject is a carrier of a genotype. The subject may be a patient, who may be diagnosed with an inflammatory disease, a fibrostenotic disease, or a fibrotic disease, such as inflammatory bowel disease (IBD) or Crohn's disease (CD). The subject may not be a patient, but may be suspected of having the inflammatory disease, the fibrostenotic disease, or the fibrotic disease. The genotype may, in some cases, be useful for treating the inflammatory fibrostenotic, or fibrotic disease or condition, as mediated by TL1A. The subject, in some embodiments, is treated by administering the inhibitor of TL1A activity or expression (e.g., anti-TL1A antibody) to the subject, provided the genotype is detected. In some cases, identifying the subject as being suitable for treatment with the inhibitor of activity or expression is required in order to administer the inhibitor to the subject.

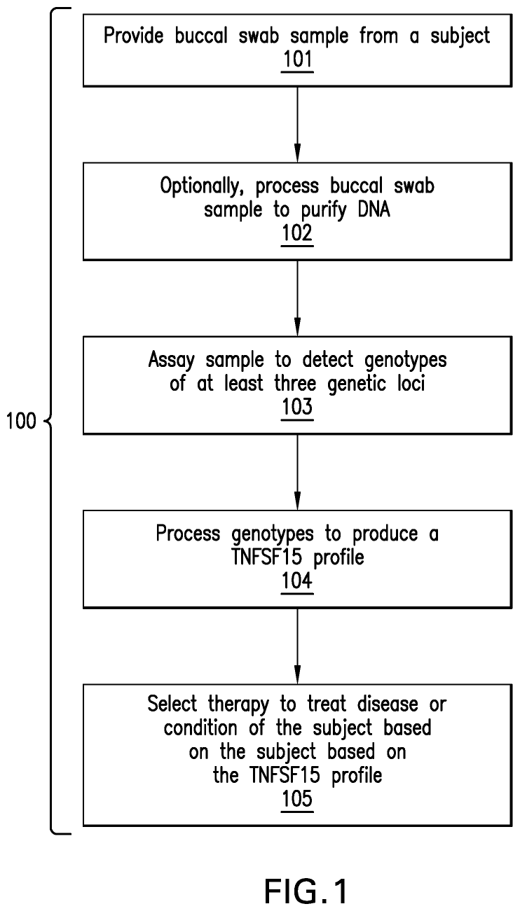

Referring to FIG. 1, the methods, systems and kits of the present disclosure involve, in some embodiments, the steps of providing a buccal swab sample from a subject 101, optionally purifying DNA from the sample by processing the sample 102, assaying the optionally processed sample to detect genotypes of at least three genetic loci in the sample 103, processing the genotypes to produce a TNFSF15 profile 104, and treating the subject with an anti-TL1A antibody or antibody fragment as disclosed herein to treat a disease or disorder of the subject based on the TNFSF15 profile 105.

The genotypes described herein are detected using suitable genotyping devices (e.g., array, sequencing). In some instances, a sample is obtained from the subject or patient indirectly or directly. In some instances, the sample may be obtained by the subject. In other instances, the sample may be obtained by a healthcare professional, such as a nurse or physician. The sample may be derived from virtually any biological fluid or tissue containing genetic information, such as blood.

The subject disclosed herein can be a mammal, such as for example a mouse, rat, guinea pig, rabbit, non-human primate, or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF alpha therapy, anti-a4-b7 therapy (vedolizumab), anti-IL12p40 therapy (ustekinumab), Thalidomide, or Cytoxin).

The disease or condition disclosed herein may be an inflammatory disease, a fibrostenotic disease, or a fibrotic disease. In some instances, the disease or the condition is a TL1A-mediated disease or condition. The term, “TL1A-mediated disease or condition” refers to a disease or a condition pathology or pathogenesis that is driven, at least in part, by TL1A signaling. In some instances, the disease or the condition is immune-mediated disease or condition, such as those mediated by TL1A.

In some embodiments the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis, rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Balb disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia—Chronic Lymphocytic (CLL), Leukemia—Chronic Myeloid (CML), Liver Cancer, Lobular Carcinoma, Lung Cancer, Lung Cancer—Small Cell (SCLC), Lung Cancer—Non-small Cell (NSCLC), Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma—Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteroids, anti-TNF therapy, anti-a4-b7 therapy (vedolizumab), anti-IL12p40 therapy (ustekinumab), Thalidomide, and Cytoxin.

### 5.1 Genotypes

Disclosed herein are genotypes that may be detected in a sample obtained from a subject by analyzing the genetic material in the sample. In some instances, the subject may be human. In some embodiments, the genetic material is obtained from a subject having a disease or condition disclosed herein. In some cases, the genetic material is obtained from blood, serum, plasma, sweat, hair, tears, urine, and other techniques known by one of skill in the art. In some cases, the genetic material is obtained from a biopsy, e.g., from the intestinal track of the subject.

The genotypes of the present disclosure comprise genetic material that is deoxyribonucleic acid (DNA). In some instances, the genotype comprises a denatured DNA molecule or fragment thereof. In some instances, the genotype comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented.

The genotypes disclosed herein comprise at least one polymorphism at a gene or genetic locus described herein. In some instances, the gene or genetic locus is selected from the group consisting of Tumor Necrosis Factor (Ligand) Superfamily, Member 15 (TNFSF15), THADA Armadillo Repeat Containing (THADA), Pleckstrin Homology, MyTH4 And FERM Domain Containing H2 (PLEKHH2), XK Related 6 (XKR6), Myotubularin Related Protein 9 (MTMR9), ETS Proto-Oncogene 1, Transcription Factor (ETS1), C-Type Lectin Domain Containing 16A (CLEC16A), Suppressor Of Cytokine Signaling 1 (SOCS1), Protein Tyrosine Phosphatase Non-Receptor Type 2 (PTPN2), Inducible T Cell Costimulator Ligand (ICOSLG), Janus Kinase 2 (JAK2), Catenin Delta 2 (CTNND2), Regulator Of G Protein Signaling 7 (RGS7), RNA Binding Fox-1 Homolog 1 (RBFOX1), RNA Binding Motif Protein 17 (RBM17), 6-Phosphofructo-2-Kinase/Fructose-2,6-Biphosphatase 3 (PFKFB3), Ecto-NOX Disulfide-Thiol Exchanger 1 (ENOX1), Coiled-Coil Domain Containing 122 (CCDC122), Regulator Of Telomere Elongation Helicase 1 (RTEL1), TNF Receptor Superfamily Member 6b (TNFRSF6B), GLIS Family Zinc Finger 3 (GLIS3), Solute Carrier Family 1 Member 1 (SLC1A1), IKAROS Family Zinc Finger 2 (IKZF2), Fatty Acyl-CoA Reductase 1 (FAR1), Spondin 1 (SPON1), Plexin A2 (PLXNA2), MIR205 Host Gene (MIR205HG), C-Type Lectin Domain Containing 16A (CLEC16A), PR/SET Domain 14 (PRDM), Autophagy Related 5 (ATG5), and Prostaglandin E Receptor 4 (PTGER4). In some instances, the gene or genetic locus comprises a gene or genetic locus provided in Table 1. The genotypes disclosed herein are, in some cases, a haplotype. In some instances, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In preferred embodiments, the genotypes disclosed herein comprise a combination of 3 polymorphisms, such as those provided in Table 1.

The polymorphisms described herein can be a single nucleotide polymorphism, or an indel (insertion/deletion). In some instances, the polymorphism is an insertion or a deletion of at least one nucleobase (e.g., an indel). In some instances, the genotype may comprise a copy number variation (CNV), which is a variation in a number of a nucleic acid sequence between individuals in a given population. In some instances, the CNV comprises at least or about two, three, four, five, six, seven, eight, nine, ten, twenty, thirty, forty or fifty nucleic acid molecules. In some instances, the genotype is heterozygous. In some instances, the genotype is homozygous.

Disclosed herein, in the following embodiments, are genotypes disclosed herein:
1. A genotype comprising at least one polymorphism at a gene or genetic locus.
2. The genotype of embodiment 1 comprising a polymorphism provided in Table 1.
3. The genotype of embodiments 1-2 that is heterozygous.
4. The genotype of embodiments 1-2 that is homozygous.
5. The genotype of embodiments 1-4, wherein the genotype comprises at least two polymorphisms.
6. The genotype of embodiments 1-4, wherein the genotype comprises at least three polymorphisms.
7. The genotype of embodiments 1-4, wherein the genotype comprises at least four polymorphisms.
8. The genotype of embodiments 1-4, wherein the genotype comprises at least five polymorphisms.
9. The genotype of embodiments 1-4, wherein the genotype comprises at least six polymorphisms.
10. The genotype of embodiments 1-4, wherein the genotype comprises at least seven polymorphisms.
11. The genotype of embodiments 1-4, wherein the genotype comprises at least eight polymorphisms.
12. The genotype of embodiment 1, comprising a polymorphism in linkage disequilibrium with a polymorphism provided in Table 1.
13. The genotype of embodiment 12, wherein LD is defined by (i) a D′ value of at least about 0.70, or (ii) a D′ value of 0 and an r² value of at least about 0.70.
14. The genotype of embodiment 12, wherein LD is defined by (i) a D′ value of at least about 0.80, or (ii) a D′ value of 0 and an r² value of at least about 0.80.
15. The genotype of embodiment 12, wherein LD is defined by (i) a D′ value of at least about 0.90, or (ii) a D′ value of 0 and an r² value of at least about 0.90.
16. The genotype of embodiment 12, wherein LD is defined by (i) a D′ value of at least about 0.95, or (ii) a D′ value of 0 and an r² value of at least about 0.95.
17. The genotype of embodiments 1-16, wherein the gene or genetic locus is selected from the group consisting of Tumor Necrosis Factor (Ligand) Superfamily, Member 15 (TNFSF15), THADA Armadillo Repeat Containing (THADA), Pleckstrin Homology, MyTH4 And FERM Domain Containing H2 (PLEKHH2), XK Related 6 (XKR6), Myotubularin Related Protein 9 (MTMR9), ETS Proto-Oncogene 1, Transcription Factor (ETS1), C-Type Lectin Domain Containing 16A (CLEC16A), Suppressor Of Cytokine Signaling 1 (SOCS1), Protein Tyrosine Phosphatase Non-Receptor Type 2 (PTPN2), Inducible T Cell Costimulator Ligand (ICOSLG), Janus Kinase 2 (JAK2), Catenin Delta 2 (CTNND2), Regulator Of G Protein Signaling 7 (RGS7), RNA Binding Fox-1 Homolog 1 (RBFOX1), RNA Binding Motif Protein 17 (RBM17), 6-Phosphofructo-2-Kinase/Fructose-2,6-Biphosphatase 3 (PFKFB3), Ecto-NOX Disulfide-Thiol Exchanger 1 (ENOX1), Coiled-Coil Domain Containing 122 (CCDC122), Regulator Of Telomere Elongation Helicase 1 (RTEL1), TNF Receptor Superfamily Member 6b (TNFRSF6B), GLIS Family Zinc Finger 3 (GLIS3), Solute Carrier Family 1 Member 1 (SLC1A1), IKAROS Family Zinc Finger 2 (IKZF2), Fatty Acyl-CoA Reductase 1 (FAR1), Spondin 1 (SPON1), Plexin A2 (PLXNA2), MIR205 Host Gene (MIR205HG), C-Type Lectin Domain Containing 16A (CLEC16A), PR/SET Domain 14 (PRDM), Autophagy Related 5 (ATG5), and Prostaglandin E Receptor 4 (PTGER4).
18. The genotype of embodiments 5-6, wherein the genotype comprises at least two polymorphisms selected from: (1) rs16901748, rs7759385, rs4246905; (2) rs16901748, rs7759385, rs7935393; (3) rs16901748, rs7759385, rs1892231; (4) rs16901748, rs7759385, rs12934476; (5) rs16901748, rs7759385, rs9806914; (6) rs16901748, rs7759385, rs2297437; (7) rs16901748, rs7759385, rs2070557; (8) rs16901748, rs7759385, rs7278257; (9) rs16901748, rs7759385, rs11221332; (10) rs16901748, rs7759385, rs41309367; (11) rs16901748, rs7759385, rs6478109; (12) rs16901748, rs4246905, rs7935393; (13) rs16901748, rs4246905, rs1892231; (14) rs16901748, rs4246905, rs12934476; (15) rs16901748, rs4246905, rs9806914; (16) rs16901748, rs4246905, rs2297437; (17) rs16901748, rs4246905, rs2070557; (18) rs16901748, rs4246905, rs7278257; (19) rs16901748, rs4246905, rs11221332; (20) rs16901748, rs4246905, rs41309367; (21) rs16901748, rs4246905, rs6478109; (22) rs16901748, rs7935393, rs1892231; (23) rs16901748, rs7935393, rs12934476; (24) rs16901748, rs7935393, rs9806914; (25) rs16901748, rs7935393, rs2297437; (26) rs16901748, rs7935393, rs2070557; (27) rs16901748, rs7935393, rs7278257; (28) rs16901748, rs7935393, rs11221332; (29) rs16901748, rs7935393, rs41309367; (30) rs16901748, rs7935393, rs6478109; (31) rs16901748, rs1892231, rs12934476; (32) rs16901748, rs1892231, rs9806914; (33) rs16901748, rs1892231, rs2297437; (34) rs16901748, rs1892231, rs2070557; (35) rs16901748, rs1892231, rs7278257; (36) rs16901748, rs1892231, rs11221332; (37) rs16901748, rs1892231, rs41309367; (38) rs16901748, rs1892231, rs6478109; (39) rs16901748, rs12934476, rs9806914; (40) rs16901748, rs12934476, rs2297437; (41) rs16901748, rs12934476, rs2070557; (42) rs16901748, rs12934476, rs7278257; (43) rs16901748, rs12934476, rs11221332; (44) rs16901748, rs12934476, rs41309367; (45) rs16901748, rs12934476, rs6478109; (46) rs16901748, rs9806914, rs2297437; (47) rs16901748, rs9806914, rs2070557; (48) rs16901748, rs9806914, rs7278257; (49) rs16901748, rs9806914, rs11221332; (50) rs16901748, rs9806914, rs41309367; (51) rs16901748, rs9806914, rs6478109; (52) rs16901748, rs2297437, rs2070557; (53) rs16901748, rs2297437, rs7278257; (54) rs16901748, rs2297437, rs11221332; (55) rs16901748, rs2297437, rs41309367; (56) rs16901748, rs2297437, rs6478109; (57) rs16901748, rs2070557, rs7278257; (58) rs16901748, rs2070557, rs11221332; (59) rs16901748, rs2070557, rs41309367; (60) rs16901748, rs2070557, rs6478109; (61) rs16901748, rs7278257, rs11221332; (62) rs16901748, rs7278257, rs41309367; (63) rs16901748, rs7278257, rs6478109; (64) rs16901748, rs11221332, rs41309367; (65) rs16901748, rs11221332, rs6478109; (66) rs16901748, rs41309367, rs6478109; (67) rs7759385, rs4246905, rs7935393; (68) rs7759385, rs4246905, rs1892231; (69) rs7759385, rs4246905, rs12934476; (70) rs7759385, rs4246905, rs9806914; (71) rs7759385, rs4246905, rs2297437; (72) rs7759385, rs4246905, rs2070557; (73) rs7759385, rs4246905, rs7278257; (74) rs7759385, rs4246905, rs11221332; (75) rs7759385, rs4246905, rs41309367; (76) rs7759385, rs4246905, rs6478109; (77) rs7759385, rs7935393, rs1892231; (78) rs7759385, rs7935393, rs12934476; (79) rs7759385, rs7935393, rs9806914; (80) rs7759385, rs7935393, rs2297437; (81) rs7759385, rs7935393, rs2070557; (82) rs7759385, rs7935393, rs7278257; (83) rs7759385, rs7935393, rs11221332; (84) rs7759385, rs7935393, rs41309367; (85) rs7759385, rs7935393, rs6478109; (86) rs7759385, rs1892231, rs12934476; (87) rs7759385, rs1892231, rs9806914; (88) rs7759385, rs1892231, rs2297437; (89) rs7759385, rs1892231, rs2070557; (90) rs7759385, rs1892231, rs7278257; (91) rs7759385, rs1892231, rs11221332; (92) rs7759385, rs1892231, rs41309367; (93) rs7759385, rs1892231, rs6478109; (94) rs7759385, rs12934476, rs9806914; (95) rs7759385, rs12934476, rs2297437; (96) rs7759385, rs12934476, rs2070557; (97) rs7759385, rs12934476, rs7278257; (98) rs7759385, rs12934476, rs11221332; (99) rs7759385, rs12934476, rs41309367; (100) rs7759385, rs12934476, rs6478109; (101) rs7759385, rs9806914, rs2297437; (102) rs7759385, rs9806914, rs2070557; (103) rs7759385, rs9806914, rs7278257; (104) rs7759385, rs9806914, rs11221332; (105) rs7759385, rs9806914, rs41309367; (106) rs7759385, rs9806914, rs6478109; (107) rs7759385, rs2297437, rs2070557; (108) rs7759385, rs2297437, rs7278257; (109) rs7759385, rs2297437, rs11221332; (110) rs7759385, rs2297437, rs41309367; (111) rs7759385, rs2297437, rs6478109; (112) rs7759385, rs2070557, rs7278257; (113) rs7759385, rs2070557, rs11221332; (114) rs7759385, rs2070557, rs41309367; (115) rs7759385, rs2070557, rs6478109; (116) rs7759385, rs7278257, rs11221332; (117) rs7759385, rs7278257, rs41309367; (118) rs7759385, rs7278257, rs6478109; (119) rs7759385, rs11221332, rs41309367; (120) rs7759385, rs11221332, rs6478109; (121) rs7759385, rs41309367, rs6478109; (122) rs4246905, rs7935393, rs1892231; (123) rs4246905, rs7935393, rs12934476; (124) rs4246905, rs7935393, rs9806914; (125) rs4246905, rs7935393, rs2297437; (126) rs4246905, rs7935393, rs2070557; (127) rs4246905, rs7935393, rs7278257; (128) rs4246905, rs7935393, rs11221332; (129) rs4246905, rs7935393, rs41309367; (130) rs4246905, rs7935393, rs6478109; (131) rs4246905, rs1892231, rs12934476; (132) rs4246905, rs1892231, rs9806914; (133) rs4246905, rs1892231, rs2297437; (134) rs4246905, rs1892231, rs2070557; (135) rs4246905, rs1892231, rs7278257; (136) rs4246905, rs1892231, rs11221332; (137) rs4246905, rs1892231, rs41309367; (138) rs4246905, rs1892231, rs6478109; (139) rs4246905, rs12934476, rs9806914; (140) rs4246905, rs12934476, rs2297437; (141) rs4246905, rs12934476, rs2070557; (142) rs4246905, rs12934476, rs7278257; (143) rs4246905, rs12934476, rs11221332; (144) rs4246905, rs12934476, rs41309367; (145) rs4246905, rs12934476, rs6478109; (146) rs4246905, rs9806914, rs2297437; (147) rs4246905, rs9806914, rs2070557; (148) rs4246905, rs9806914, rs7278257; (149) rs4246905, rs9806914, rs11221332; (150) rs4246905, rs9806914, rs41309367; (151) rs4246905, rs9806914, rs6478109; (152) rs4246905, rs2297437, rs2070557; (153) rs4246905, rs2297437, rs7278257; (154) rs4246905, rs2297437, rs11221332; (155) rs4246905, rs2297437, rs41309367; (156) rs4246905, rs2297437, rs6478109; (157) rs4246905, rs2070557, rs7278257; (158) rs4246905, rs2070557, rs11221332; (159) rs4246905, rs2070557, rs41309367; (160) rs4246905, rs2070557, rs6478109; (161) rs4246905, rs7278257, rs11221332; (162) rs4246905, rs7278257, rs41309367; (163) rs4246905, rs7278257, rs6478109; (164) rs4246905, rs11221332, rs41309367; (165) rs4246905, rs11221332, rs6478109; (166) rs4246905, rs41309367, rs6478109; (167) rs7935393, rs1892231, rs12934476; (168) rs7935393, rs1892231, rs9806914; (169) rs7935393, rs1892231, rs2297437; (170) rs7935393, rs1892231, rs2070557; (171) rs7935393, rs1892231, rs7278257; (172) rs7935393, rs1892231, rs11221332; (173) rs7935393, rs1892231, rs41309367; (174) rs7935393, rs1892231, rs6478109; (175) rs7935393, rs12934476, rs9806914; (176) rs7935393, rs12934476, rs2297437; (177) rs7935393, rs12934476, rs2070557; (178) rs7935393, rs12934476, rs7278257; (179) rs7935393, rs12934476, rs11221332; (180) rs7935393, rs12934476, rs41309367; (181) rs7935393, rs12934476, rs6478109; (182) rs7935393, rs9806914, rs2297437; (183) rs7935393, rs9806914, rs2070557; (184) rs7935393, rs9806914, rs7278257; (185) rs7935393, rs9806914, rs11221332; (186) rs7935393, rs9806914, rs41309367; (187) rs7935393, rs9806914, rs6478109; (188) rs7935393, rs2297437, rs2070557; (189) rs7935393, rs2297437, rs7278257; (190) rs7935393, rs2297437, rs11221332; (191) rs7935393, rs2297437, rs41309367; (192) rs7935393, rs2297437, rs6478109; (193) rs7935393, rs2070557, rs7278257; (194) rs7935393, rs2070557, rs11221332; (195) rs7935393, rs2070557, rs41309367; (196) rs7935393, rs2070557, rs6478109; (197) rs7935393, rs7278257, rs11221332; (198) rs7935393, rs7278257, rs41309367; (199) rs7935393, rs7278257, rs6478109; (200) rs7935393, rs11221332, rs4130936;7 (201) rs7935393, rs11221332, rs6478109; (202) rs7935393, rs41309367, rs6478109; (203) rs1892231, rs12934476, rs9806914; (204) rs1892231, rs12934476, rs2297437; (205) rs1892231, rs12934476, rs2070557; (206) rs1892231, rs12934476, rs7278257; (207) rs1892231, rs12934476, rs11221332; (208) rs1892231, rs12934476, rs41309367; (209) rs1892231, rs12934476, rs6478109; (210) rs1892231, rs9806914, rs2297437; (211) rs1892231, rs9806914, rs2070557; (212) rs1892231, rs9806914, rs7278257; (213) rs1892231, rs9806914, rs11221332; (214) rs1892231, rs9806914, rs41309367; (215) rs1892231, rs9806914, rs6478109; (216) rs1892231, rs2297437, rs2070557; (217) rs1892231, rs2297437, rs7278257; (218) rs1892231, rs2297437, rs11221332; (219) rs1892231, rs2297437, rs41309367; (220) rs1892231, rs2297437, rs6478109; (221) rs1892231, rs2070557, rs7278257; (222) rs1892231, rs2070557, rs11221332; (223) rs1892231, rs2070557, rs41309367; (224) rs1892231, rs2070557, rs6478109; (225) rs1892231, rs7278257, rs11221332; (226) rs1892231, rs7278257, rs41309367; (227) rs1892231, rs7278257, rs6478109; (228) rs1892231, rs11221332, rs41309367; (229) rs1892231, rs11221332, rs6478109; (230) rs1892231, rs41309367, rs6478109; (231) rs12934476, rs9806914, rs2297437; (232) rs12934476, rs9806914, rs2070557; (233) rs12934476, rs9806914, rs7278257; (234) rs12934476, rs9806914, rs11221332; (235) rs12934476, rs9806914, rs41309367; (236) rs12934476, rs9806914, rs6478109; (237) rs12934476, rs2297437, rs2070557; (238) rs12934476, rs2297437, rs7278257; (239) rs12934476, rs2297437, rs11221332; (240) rs12934476, rs2297437, rs41309367; (241) rs12934476, rs2297437, rs6478109; (242) rs12934476, rs2070557, rs7278257; (243) rs12934476, rs2070557, rs11221332; (244) rs12934476, rs2070557, rs41309367; (245) rs12934476, rs2070557, rs6478109; (246) rs12934476, rs7278257, rs11221332; (247) rs12934476, rs7278257, rs41309367; (248) rs12934476, rs7278257, rs6478109; (249) rs12934476, rs11221332, rs41309367; (250) rs12934476, rs11221332, rs6478109; (251) rs12934476, rs41309367, rs6478109; (252) rs9806914, rs2297437, rs2070557; (253) rs9806914, rs2297437, rs7278257; (254) rs9806914, rs2297437, rs11221332; (255) rs9806914, rs2297437, rs41309367; (256) rs9806914, rs2297437, rs6478109; (257) rs9806914, rs2070557, rs7278257; (258) rs9806914, rs2070557, rs11221332; (259) rs9806914, rs2070557, rs41309367; (260) rs9806914, rs2070557, rs6478109; (261) rs9806914, rs7278257, rs11221332; (262) rs9806914, rs7278257, rs41309367; (263) rs9806914, rs7278257, rs6478109; (264) rs9806914, rs11221332, rs41309367; (265) rs9806914, rs11221332, rs6478109; (266) rs9806914, rs41309367, rs6478109; (267) rs2297437, rs2070557, rs7278257; (268) rs2297437, rs2070557, rs11221332; (269) rs2297437, rs2070557, rs41309367; (270) rs2297437, rs2070557, rs6478109; (271) rs2297437, rs7278257, rs11221332; (272) rs2297437, rs7278257, rs41309367; (273) rs2297437, rs7278257, rs6478109; (274) rs2297437, rs11221332, rs41309367; (275) rs2297437, rs11221332, rs6478109; (276) rs2297437, rs41309367, rs6478109; (277) rs2070557, rs7278257, rs11221332; (278) rs2070557, rs7278257, rs41309367; (279) rs2070557, rs7278257, rs6478109; (280) rs2070557, rs11221332, rs41309367; (281) rs2070557, rs11221332, rs6478109; (282) rs2070557, rs41309367, rs6478109; (283) rs7278257, rs11221332, rs41309367; (284) rs7278257, rs11221332, rs6478109; (285) rs7278257, rs41309367, rs6478109; or (286) rs11221332, rs41309367, rs6478109.
19. The genotype of embodiment 18, wherein the rs7278257 is replaced with rs56124762.
20. The genotype of embodiment 18, wherein the rs7278257 is replaced with rs2070558.
21. The genotype of embodiment 18, wherein the rs7278257 is replaced with rs2070561.
22. The genotype of embodiments 5-6, wherein the genotype comprises at least two polymorphisms selected from imm_9_116608587, imm_11_127948309, and rs1892231.
23. The genotype of embodiments 5-6, wherein the genotype comprises at least two polymorphisms selected from imm_9_116608587, imm_11_127948309, and rs9806914.
24. The genotype of embodiments 5-6, wherein the genotype comprises at least two polymorphisms selected from imm_9_116608587, imm_11_127948309, and imm_21_44478192.
25. The genotype of embodiments 5-6, wherein the genotype comprises at least two polymorphisms selected from imm_9_116608587, imm_11_127948309, and imm_21_44479552.
26. The genotype of embodiments 5-6, wherein the genotype comprises at least two polymorphisms selected from imm_9_116608587, rs1892231, and rs9806914.
27. The genotype of embodiments 5-6, wherein the genotype comprises at least two polymorphisms selected from imm_9_116608587, rs1892231, and imm_21_44478192.
28. The genotype of embodiments 5-6, wherein the genotype comprises at least two polymorphisms selected from imm_9_116608587, rs1892231, and imm_21_44479552.
29. The genotype of embodiments 5-6, wherein the genotype comprises at least two polymorphisms selected from imm_9_116608587, rs9806914, and imm_21_44478192.
30. The genotype of embodiments 5-6, wherein the genotype comprises at least two polymorphisms selected from imm_9_116608587, rs9806914, and imm_21_44479552.
31. The genotype of embodiments 5-6, wherein the genotype comprises at least two polymorphisms selected from imm_9_116608587, imm_21_44478192, and imm_21_44479552.
32. The genotype of embodiments 5-6, wherein the genotype comprises at least two polymorphisms selected from imm_11_127948309, rs1892231, and rs9806914.
33. The genotype of embodiments 5-6, wherein the genotype comprises at least two polymorphisms selected from imm_11_127948309, rs1892231, and imm_21_44478192.
34. The genotype of embodiments 5-6, wherein the genotype comprises at least two polymorphisms selected from imm_11_127948309, rs1892231, and imm_21_44479552.
35. The genotype of embodiments 5-6, wherein the genotype comprises at least two polymorphisms selected from imm_11_127948309, rs9806914, and imm_21_44478192.
36. The genotype of embodiments 5-6, wherein the genotype comprises at least two polymorphisms selected from imm_11_127948309, rs9806914, and imm_21_44479552.
37. The genotype of embodiments 5-6, wherein the genotype comprises at least two polymorphisms selected from imm_11_127948309, imm_21_44478192, and imm_21_44479552.
38. The genotype of embodiments 5-6, wherein the genotype comprises at least two polymorphisms selected from rs1892231, rs9806914, and imm_21_44478192.
39. The genotype of embodiments 5-6, wherein the genotype comprises at least two polymorphisms selected from rs1892231, rs9806914, and imm_21_44479552.
40. The genotype of embodiments 5-6, wherein the genotype comprises at least two polymorphisms selected from rs1892231, imm_21_44478192, and imm_21_44479552.
41. The genotype of embodiments 5-6, wherein the genotype comprises at least two polymorphisms selected from rs9806914, imm_21_44478192, and imm_21_44479552.
42. The genotype of embodiments 5-6, wherein the genotype comprises at least two polymorphisms selected from rs6478109, rs56124762, and rs1892231.
43. The genotype of embodiments 5-6, wherein the genotype comprises at least two polymorphisms selected from rs6478109, rs56124762, and rs16901748.
44. The genotype of embodiments 5-6, wherein the genotype comprises at least two polymorphisms selected from rs6478109, rs1892231, and rs16901748.
45. The genotype of embodiments 5-6, wherein the genotype comprises at least two polymorphisms selected from rs56124762, rs1892231, and rs16901748.
46. The genotype of embodiments 5-6, wherein the genotype comprises at least two polymorphisms selected from rs6478109, rs2070558, and rs1892231. 47. The genotype of embodiments 5-6, wherein the genotype comprises at least two polymorphisms selected from rs6478109, rs2070558, and rs16901748.
48. The genotype of embodiments 5-6, wherein the genotype comprises at least two polymorphisms selected from rs6478109, rs1892231, and rs16901748.
49. The genotype of embodiments 5-6, wherein the genotype comprises at least two polymorphisms selected from rs2070558, rs1892231, and rs16901748.
50. The genotype of embodiments 5-6, wherein the genotype comprises at least two polymorphisms selected from rs6478109, rs2070561, and rs1892231.
51. The genotype of embodiments 5-6, wherein the genotype comprises at least two polymorphisms selected from rs6478109, rs2070561, and rs16901748.
52. The genotype of embodiments 5-6, wherein the genotype comprises at least two polymorphisms selected from rs6478109, rs1892231, and rs16901748.
53. The genotype of embodiments 5-6, wherein the genotype comprises at least two polymorphisms selected from rs2070561, rs1892231, and rs16901748.
54. The genotype of embodiment 11, wherein the genotype comprises eight polymorphisms selected from any 8-SNP combination of Model_1 to Model_495 described in Table 25.
55. The genotype of embodiments 1-53, wherein the genotype comprises a minor allele provided in Table 1 for at least one polymorphism.
56. The genotype of embodiments 1-53, wherein the genotype comprises a major allele provided in Table 1 for at least one polymorphism.
57. The genotype of embodiments 1-56, wherein a presence of the genotype is predictive of a positive therapeutic response of IBD patients to a treatment with an inhibitor of TL1A activity of expression at a positive predictive value of at least about 25%, 26%, 27%, 28%, 29%, 30%, 31%, 32%, 33%, 34%, 35%, 40%, 45%, 50%, 55%, 60%, 65%, 70%, 75%, 80%, 85%, 90%, 91%, 92%, 93%, 94%, 95%, 96%, 97%, 98%, 99%, or 100%.
58. The genotype of embodiments 1-57, wherein a presence of the genotype is predictive of a positive therapeutic response of IBD patients to a treatment with an inhibitor of TL1A activity of expression with a specificity of at least about 50%, 55%, 60%, 65%, 70%, 75%, 80%, 85%, 90%, 91%, 92%, 93%, 94%, 95%, 96%, 97%, 98%, 99%, or 100%.
59. The genotype of embodiments 1-58, wherein a presence of the genotype is predictive of a positive therapeutic response of IBD patients to a treatment with an inhibitor of TL1A activity of expression with a negative predictive value of at least about 50%, 55%, 60%, 65%, 70%, 75%, 80%, 85%, 90%, 91%, 92%, 93%, 94%, 95%, 96%, 97%, 98%, 99%, or 100%.
60. The genotype of embodiments 1-59, wherein a presence of the genotype is predictive of a positive therapeutic response of IBD patients to a treatment with an inhibitor of TL1A activity of expression with a sensitivity of at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100%.
61. The genotype of embodiments 1-60, wherein a presence of the genotype is predictive of a positive therapeutic response of IBD patients to a treatment with an inhibitor of TL1A activity of expression with an accuracy of at least about 50%, 55%, 60%, 65%, 70%, 75%, 80%, 85%, 90%, 91%, 92%, 93%, 94%, 95%, 96%, 97%, 98%, 99%, or 100%.
62. The genotype of embodiments 1-61, wherein a presence of the genotype is predictive of a positive therapeutic response of IBD patients to a treatment with an inhibitor of TL1A activity of expression with positive rate of at least about 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, or 70%.

Aspects disclosed herein provide genotypes that are associated with, and therefore indicative of, a subject having or being susceptible to developing a particular disease or condition, or a subclinical phenotype thereof. In addition, the genotypes disclosed herein are associated with an increase TNFSF15 (TL1A) expression or activity. Thus, the genotypes are indicative that the subject will have a positive therapeutic response to an inhibitor of TL1A activity or expression. Table 1 provides exemplary polymorphisms associated with, and therefore predictive of, a positive therapeutic response to an inhibitor of TNFSF15 (TL1A) expression or activity. The term, “positive therapeutic response” refers to a reduction or an elimination of at least one symptom of the disease or the condition (e.g., Cohn's disease) after induction of a therapy (e.g., anti-TL1A antibody).

TABLE 1
Exemplary Polymorphisms
Table 1. Exemplary Polymorphisms

| | | | Minor | Major | |
|---|---|---|---|---|---|
| | | | Allele (or | Allele (or | |
| | | | referred to as | referred to as | |
| | | | Alternative, | Reference, | |
| | | | ALT, Alt | REF, Ref | SEQ ID |
| rsID | Chip_id | Gene | Allele) | Allele) | NO |
| rs11897732 | lkg_2_43394890 | THADA | G | A | 2001 |
| rs6740739 | lkg_2_43709147 | THADA, PLEKHH2 | A | G | 2002 |
| rs17796285 | lkg_8_11161865 | XKR6, MTMR9 | G | C | 2003 |
| rs7935393 | imm_11_127948309 | ETS1 | C | A | 2004 |
| rs12934476 | imm_16_11239010 | CLEC16A, SOCS1 | G | A | 2005 |
| rs12457255 | imm_18_12749976 | LOC100996324, PTPN2 | A | C | 2006 |
| rs2070557 | imm_21_44479552 | ICOSLG | A | T | 2007 |
| rs4246905 | imm_9_116593070 | TNFSF15 | A | G | 2008 |
| rs10974900 | imm_9_4977958 | JAK2 | A | G | 2009 |
| rs12434976 | rs12434976 | LINC01550, C14orf177 | C | A | 2010 |
| rs16901748 | rs16901748 | CTNND2 | T | G | 2011 |
| rs2815844 | rs2815844 | RGS7 | A | G | 2012 |
| rs889702 | rs889702 | RBFOX1 | G | A | 2013 |
| rs2409750 | lkg_8_11125104 | XKR6, MTMR9 | C | A | 2014 |
| rs1541020 | imm_10_6205036 | RBM17, PFKFB3 | A | G | 2015 |
| rs4942248 | imm_13_43304805 | ENOX1, CCDC122 | T | A | 2016 |
| rs12934476 | imm_16_11239010 | CLEC16A, SOCS1 | G | A | 2017 |
| rs12457255 | imm_18_12749976 | LOC100996324, PTPN2 | A | C | 2018 |
| rs2297437 | imm_20_61775718 | RTEL1-TNFRSF6B | A | G | 2019 |
| rs41309367 | imm_20_61779998 | RTEL1-TNFRSF6B | G | A | 2020 |
| rs10733509 | imm_9_4298050 | GLIS3, SLC1A1 | A | G | 2021 |
| rs10750376 | rs10750376 | LOC101929497, ETS1 | G | A | 2022 |
| rs10932456 | rs10932456 | MIR4776-2, IKZF2 | G | A | 2023 |
| rs1326860 | rs1326860 | LINC01031, NONE | A | G | 2024 |
| rs1528663 | rs1528663 | FAR1, SPON1 | G | A | 2025 |
| rs1892231 | rs1892231 | LINC01550, C14orf177 | C | A | 2026 |
| rs951279 | rs951279 | PLXNA2, MIR205HG | G | A | 2027 |
| rs9806914 | rs9806914 | RBFOX1 | A | G | 2028 |
| rs7935393 | imm_11_127948309 | ETS1 | C | A | 2029 |
| rs1690492 | imm_16_11226317 | CLEC16A, SOCS1 | G | C | 2030 |
| rs420726 | imm_21_44483873 | ICOSLG | G | A | 2031 |
| rs7759385 | imm_6_106695463 | PRDM1, ATG5 | T | A | 2032 |
| rs10974900 | imm_9_4977958 | JAK2 | A | G | 2033 |
| rs1326860 | rs1326860 | LINC01031. NONE | A | G | 2034 |
| rs2548147 | rs2548147 | LINC00603, PTGER4 | C | G | 2035 |
| rs2815844 | rs2815844 | RGS7 | A | G | 2036 |
| rs889702 | rs889702 | RBFOX1 | G | A | 2037 |
| rs9806914 | rs9806914 | RBFOX1 | A | G | 2038 |
| rs6478109 | imm_9_116608587 | TNFSF15 | A | G | 2039 |
| rs7278257 | imm_21_44478192 | ICOSLG | C | G | 2040 |
| rs11221332 | imm_11_127886184 | ETS1 | A | G | 2041 |
| rs56124762 | imm_21_44482902 | ICOSLG | G | A | 2057 |
| rs2070558 | imm_21_44480086 | ICOSLG | G | A | 2058 |
| rs2070561 | rs2070561 | ICOSLG | T | C | 2059 |

The instant disclosure provides models comprising 3 polymorphisms (e.g., “3-SNP Models”) that, when detected in a sample obtained from a subject, indicate a positive therapeutic response in the subject to a treatment, such as with an inhibitor of TL1A activity or expression. Non-limiting examples of models described herein include Model A (rs6478109, rs7278257, and rs1892231); Model B (rs6478109, rs2070557, and rs9806914); Model C (rs6478109, rs7935393, and rs1892231); Model D (rs6478109, rs7935393, and rs9806914); Model E (rs6478109, rs9806914, and rs16901748); Model F (rs6478109, rs16901748, and rs2297437); Model G (rs6478109, rs1892231, and rs16901748); Model H (rs6478109, rs2070557, and rs7935393); Model I (rs6478109, rs7278257, and rs7935393); Model J (rs6478109, rs9806914, and rs1892231); and Model K (rs6478109, rs7278257, and rs16901748).

The instant disclosure provides models comprising 1, 2, 3, 4, 5, 6, 7, 8, 9, 10 or more polymorphisms (e.g., “1-SNP Model,” “2-SNP Models” or “3-SNP Models,” etc.) that, when detected in a sample obtained from a subject, indicate a positive therapeutic response in the subject to a treatment, such as with an inhibitor of TL1A activity or expression. Non-limiting examples of models described herein include the 10-SNP, 9-SNP, 8-SNP, 7-SNP, 6-SNP, 5-SNP, 4-SNP, 3-SNP, 2-SNP, or 1-SNP models described in Table 31.

The genotypes and/or the polymorphisms provided herein in Table 1 have already been validated as the genotypes and/or the polymorphisms that correlate with increased increased TL1A expression in inflammatory cells, with increased inflammation, with phenotypes of IBD, with an increase of IBD enriched cell types, with a decrease of IBD depleted cell types, and/or with an increase of a positive therapeutic response in IBD patients to a treatment with the inhibitor of TL1A activity or expression, as further described in Section 7 (EXAMPLES). As such, the disclosure provides that the genotypes and/or the polymorphisms, the combinations of genotypes, and/or the combinations of polymorphisms provided herein can be used as criteria for identifying subjects or patients for the various methods provided herein including in Sections 2, 5.2, and 7 (EXAMPLES). Similarly, the disclosure provides that the genotypes and/or the polymorphisms, the combinations of genotypes, and/or the combinations of polymorphisms provided herein can be used as criteria for identifying subjects or patients for the various kits and compositions provided herein including in Sections 2, 5.5, 5.7, and 7 (EXAMPLES). Additionally, the disclosure provides that the genotypes and/or the polymorphisms, the combinations of genotypes, and/or the combinations of polymorphisms provided herein can be used as criteria for the various methods of selecting patients provided herein including in Sections 2, 5.2, and 7.

The disclosure further provides simple methods to validate the suitability of the genotypes and/or polymorphisms, the combination of genotypes, and/or the combination of polymorphisms for the various methods of treatment, methods of selecting patients, and/or the kits and compositions provided herein including in Sections 2, 5.2, 5.5, 5.7, and 7.

The polymorphisms identified in the analysis provided in the Examples above may be used to predict a positive therapeutic response in a subject or a patient to an inhibitor of TL1A activity or expression (e.g., anti-TL1A antibody), either alone, or in combinations (e.g., 2, 3, 4, 5, 6, 7, 8, 9, 10, or more). The polymorphisms described herein may be used in a diagnostic or prognostic test to identify a subject suitable for treatment with an inhibitor of TL1A activity or expression to treat a disease or condition described herein in the subject. In some cases, the diagnostic is a companion diagnostic test, such as for example, a TL1A companion diagnostic test (“TL1A CDx”).

To validate the rules and polymorphisms, an external cohort of IBD (e.g., UC or CD) patients can be identified and genotyped. The positive predictive value, negative predictive value, specificity, sensitivity, and positive rate of the patient population can be calculated from the genotypes of the patients and the clinical response or clinical remission of the IBD patients after the TL1A inhibitor therapy, for example as described in Section 7 (Examples). The disclosure provides that because the polymorphisms provided herein were already selected via the machine learning algorithms provided herein (e.g., in the preceding few paragraphs and in Section 7 (Examples)) to be associated with IBD readouts (e.g., TL1A expression, TL1A activity, IBD phenotypes, an increase of IBD enriched cell types, with a decrease of IBD depleted cell types, patient clinical response, and patient clinical remission, as described in Section 7 (Examples)), the rules and combinations of polymorphisms for a given positive predictive value, negative predictive value, specificity, sensitivity, and/or positive rate can be identified by the machine learning algorithms and validated as provided herein without undue experimentation.

In some embodiments of the methods provided herein, the genotype comprises polymorphism. In certain embodiments of the methods provided herein, the polymorphism comprises SNP. In some embodiments of the methods provided herein, the combination of genotype comprises a combination of polymorphisms. In certain embodiments of the methods provided herein, the combination of polymorphisms comprises a combination of SNPs.

Table 1 and Table 27 provides exemplary SNPs predictive of clinical response or remission in patients treated with TL1A inhibitor therapy. The disclosure provides that the various combinations of polymorphisms predictive of a positive therapeutic response to the inhibitor of the TL1A activity or expression comprise combinations of polymorphisms predictive of elevated TL1A expression, combinations of polymorphisms predictive of elevated TL1A transcriptome, or combinations of both polymorphisms predictive of elevated TL1A expression and polymorphisms predictive of elevated TL1A transcriptome. The disclosure also provides that the various combinations of polymorphisms predictive of a positive therapeutic response to the inhibitor of the TL1A activity or expression comprise combinations of polymorphisms predictive of clinical response or remission in patients treated with TL1A inhibitor therapy, as listed in Table 1 and Table 27. The disclosure additionally provides that the various combinations of polymorphisms predictive of a positive therapeutic response to the inhibitor of the TL1A activity or expression comprise combinations of polymorphisms predictive of clinical response or remission in patients treated with TL1A inhibitor therapy, as listed in Table 1 and Table 27.

Accordingly, in one embodiment, the combination of polymorphisms predictive of a positive therapeutic response to the inhibitor of the TL1A activity or expression comprises a combination of 2 polymorphisms predictive of elevated TL1A expression. In one embodiment, the combination of polymorphisms predictive of a positive therapeutic response to the inhibitor of the TL1A activity or expression comprises a combination of 3 polymorphisms predictive of elevated TL1A expression. In one embodiment, the combination of polymorphisms predictive of a positive therapeutic response to the inhibitor of the TL1A activity or expression comprises a combination of 4 polymorphisms predictive of elevated TL1A expression. In one embodiment, the combination of polymorphisms predictive of a positive therapeutic response to the inhibitor of the TL1A activity or expression comprises a combination of 5 polymorphisms predictive of elevated TL1A expression. In one embodiment, the combination of polymorphisms predictive of a positive therapeutic response to the inhibitor of the TL1A activity or expression comprises a combination of 6 polymorphisms predictive of elevated TL1A expression. In one embodiment, the combination of polymorphisms predictive of a positive therapeutic response to the inhibitor of the TL1A activity or expression comprises a combination of 7 polymorphisms predictive of elevated TL1A expression. In one embodiment, the combination of polymorphisms predictive of a positive therapeutic response to the inhibitor of the TL1A activity or expression comprises a combination of 8 polymorphisms predictive of elevated TL1A expression. In one embodiment, the combination of polymorphisms predictive of a positive therapeutic response to the inhibitor of the TL1A activity or expression comprises a combination of 9 polymorphisms predictive of elevated TL1A expression. In one embodiment, the combination of polymorphisms predictive of a positive therapeutic response to the inhibitor of the TL1A activity or expression comprises a combination of 10 polymorphisms predictive of elevated TL1A expression. In one embodiment, the polymorphism predictive of a positive therapeutic response to the inhibitor of the TL1A activity or expression comprises 1 polymorphism predictive of elevated TL1A expression. In some embodiments, the polymorphism or the combinations of polymorphisms predictive of elevated TL1A expression provided herein for the various methods (including in this paragraph) are selected from Table 27 (SEQ ID NOS: 2004-2006, 2009, 2011, 2012, 2014-2016, 2019, 2024, 2026, 2028, 2032, 2039, and 2057). In some embodiments, the polymorphism or the combinations of polymorphisms predictive of elevated TL1A expression provided herein for the various methods (including in this paragraph) are selected from Table 1 (SEQ ID NOS: 2001-2041 and 2057-2059).

Alternatively, in one embodiment, the combination of polymorphisms predictive of a positive therapeutic response to the inhibitor of the TL1A activity or expression comprises a combination of 2 polymorphisms predictive of elevated TL1A activity. In one embodiment, the combination of polymorphisms predictive of a positive therapeutic response to the inhibitor of the TL1A activity or expression comprises a combination of 3 polymorphisms predictive of elevated TL1A activity. In one embodiment, the combination of polymorphisms predictive of a positive therapeutic response to the inhibitor of the TL1A activity or expression comprises a combination of 4 polymorphisms predictive of elevated TL1A activity. In one embodiment, the combination of polymorphisms predictive of a positive therapeutic response to the inhibitor of the TL1A activity or expression comprises a combination of 5 polymorphisms predictive of elevated TL1A activity. In one embodiment, the combination of polymorphisms predictive of a positive therapeutic response to the inhibitor of the TL1A activity or expression comprises a combination of 6 polymorphisms predictive of elevated TL1A activity. In one embodiment, the combination of polymorphisms predictive of a positive therapeutic response to the inhibitor of the TL1A activity or expression comprises a combination of 7 polymorphisms predictive of elevated TL1A activity. In one embodiment, the combination of polymorphisms predictive of a positive therapeutic response to the inhibitor of the TL1A activity or expression comprises a combination of 8 polymorphisms predictive of elevated TL1A activity. In one embodiment, the combination of polymorphisms predictive of a positive therapeutic response to the inhibitor of the TL1A activity or expression comprises a combination of 9 polymorphisms predictive of elevated TL1A activity. In one embodiment, the combination of polymorphisms predictive of a positive therapeutic response to the inhibitor of the TL1A activity or expression comprises a combination of 10 polymorphisms predictive of elevated TL1A activity. In one embodiment, the polymorphism predictive of a positive therapeutic response to the inhibitor of the TL1A activity or expression comprises 1 polymorphism predictive of elevated TL1A activity. In some embodiments, the polymorphism or the combinations of polymorphisms predictive of elevated TL1A activity provided herein for the various methods (including in this paragraph) are selected from Table 27 (SEQ ID NOS: 2004-2006, 2009, 2011, 2012, 2014-2016, 2019, 2024, 2026, 2028, 2032, 2039, and 2057). In some embodiments, the polymorphism or the combinations of polymorphisms predictive of elevated TL1A activity provided herein for the various methods (including in this paragraph) are selected from Table 1 (SEQ ID NOS: 2001-2041 and 2057-2059).

Additionally, in one embodiment, the combination of polymorphisms predictive of a positive therapeutic response to the inhibitor of the TL1A activity or expression comprises a combination of 2 polymorphisms predictive of an increase in IBD enriched cell types. In one embodiment, the combination of polymorphisms predictive of a positive therapeutic response to the inhibitor of the TL1A activity or expression comprises a combination of 3 polymorphisms predictive of an increase in IBD enriched cell types. In one embodiment, the combination of polymorphisms predictive of a positive therapeutic response to the inhibitor of the TL1A activity or expression comprises a combination of 4 polymorphisms predictive of an increase in IBD enriched cell types. In one embodiment, the combination of polymorphisms predictive of a positive therapeutic response to the inhibitor of the TL1A activity or expression comprises a combination of 5 polymorphisms predictive of an increase in IBD enriched cell types. In one embodiment, the combination of polymorphisms predictive of a positive therapeutic response to the inhibitor of the TL1A activity or expression comprises a combination of 6 polymorphisms predictive of an increase in IBD enriched cell types. In one embodiment, the combination of polymorphisms predictive of a positive therapeutic response to the inhibitor of the TL1A activity or expression comprises a combination of 7 polymorphisms predictive of an increase in IBD enriched cell types. In one embodiment, the combination of polymorphisms predictive of a positive therapeutic response to the inhibitor of the TL1A activity or expression comprises a combination of 8 polymorphisms predictive of an increase in IBD enriched cell types. In one embodiment, the combination of polymorphisms predictive of a positive therapeutic response to the inhibitor of the TL1A activity or expression comprises a combination of 9 polymorphisms predictive of an increase in IBD enriched cell types. In one embodiment, the combination of polymorphisms predictive of a positive therapeutic response to the inhibitor of the TL1A activity or expression comprises a combination of 10 polymorphisms predictive of an increase in IBD enriched cell types. In one embodiment, the polymorphism predictive of a positive therapeutic response to the inhibitor of the TL1A activity or expression comprises 1 polymorphism predictive of an increase in IBD enriched cell types. In some embodiments, the polymorphism or the combinations of polymorphisms predictive of an increase in IBD enriched cell types provided herein for the various methods (including in this paragraph) are selected from Table 27 (SEQ ID NOS: SEQ ID NOS: 2004-2006, 2009, 2011, 2012, 2014-2016, 2019, 2024, 2026, 2028, 2032, 2039, and 2057). In some embodiments, the polymorphism or the combinations of polymorphisms predictive of an increase in IBD enriched cell types provided herein for the various methods (including in this paragraph) are selected from Table 1 (SEQ ID NOS: 2001-2041 and 2057-2059).

Furthermore, in one embodiment, the combination of polymorphisms predictive of a positive therapeutic response to the inhibitor of the TL1A activity or expression comprises a combination of 2 polymorphisms predictive of a decrease in IBD depleted cell types. In one embodiment, the combination of polymorphisms predictive of a positive therapeutic response to the inhibitor of the TL1A activity or expression comprises a combination of 3 polymorphisms predictive of a decrease in IBD depleted cell types. In one embodiment, the combination of polymorphisms predictive of a positive therapeutic response to the inhibitor of the TL1A activity or expression comprises a combination of 4 polymorphisms predictive of a decrease in IBD depleted cell types. In one embodiment, the combination of polymorphisms predictive of a positive therapeutic response to the inhibitor of the TL1A activity or expression comprises a combination of 5 polymorphisms predictive of a decrease in IBD depleted cell types. In one embodiment, the combination of polymorphisms predictive of a positive therapeutic response to the inhibitor of the TL1A activity or expression comprises a combination of 6 polymorphisms predictive of a decrease in IBD depleted cell types. In one embodiment, the combination of polymorphisms predictive of a positive therapeutic response to the inhibitor of the TL1A activity or expression comprises a combination of 7 polymorphisms predictive of a decrease in IBD depleted cell types. In one embodiment, the combination of polymorphisms predictive of a positive therapeutic response to the inhibitor of the TL1A activity or expression comprises a combination of 8 polymorphisms predictive of a decrease in IBD depleted cell types. In one embodiment, the combination of polymorphisms predictive of a positive therapeutic response to the inhibitor of the TL1A activity or expression comprises a combination of 9 polymorphisms predictive of a decrease in IBD depleted cell types. In one embodiment, the combination of polymorphisms predictive of a positive therapeutic response to the inhibitor of the TL1A activity or expression comprises a combination of 10 polymorphisms predictive of a decrease in IBD depleted cell types. In one embodiment, the polymorphism predictive of a positive therapeutic response to the inhibitor of the TL1A activity or expression comprises 1 polymorphism predictive of a decrease in IBD depleted cell types. In some embodiments, the polymorphism or the combinations of polymorphisms predictive of a decrease in IBD depleted cell types provided herein for the various methods (including in this paragraph) are selected from Table 27 (SEQ ID NOS: SEQ ID NOS: 2004-2006, 2009, 2011, 2012, 2014-2016, 2019, 2024, 2026, 2028, 2032, 2039, and 2057). In some embodiments, the polymorphism or the combinations of polymorphisms predictive of a decrease in IBD depleted cell types provided herein for the various methods (including in this paragraph) are selected from Table 1 (SEQ ID NOS: 2001-2041 and 2057-2059).

In some embodiments, the elevated TL1A expression, elevated TL1A activity, increase of IBD enriched cell types, and decrease of IBD depleted cell types in this Section (including in the preceding paragraphs) are in reference to such in a tissue or subject not affected by IBD (e.g. UC or CD).

### 5.2 Methods of Treatment

Disclosed herein are methods of treating a disease or condition, or a symptom of the disease or condition, in a subject, comprising administrating of therapeutic effective amount of one or more therapeutic agents to the subject. In some embodiments, the one or more therapeutic agents is administered to the subject alone (e.g., standalone therapy). In some embodiments, the one or more therapeutic agents is administered in combination with an additional agent. In some embodiments, the therapeutic agent is a first-line therapy for the disease or condition. In some embodiments, the therapeutic agent is a second-line, third-line, or fourth-line therapy, for the disease or condition. In some embodiments, the therapeutic agent comprises an inhibitor of Tumor necrosis factor-like cytokine 1A (TL1A) activity or expression.

Various embodiments provide for methods of treating inflammatory bowel disease (IBD), comprising administering an anti-TL1A antibody described herein to a subject in need thereof. In some embodiments, the subject comprises one or more risk genotypes. In some embodiments, the IBD is a severe form of IBD.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of polymorphisms determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI above a cutoff indicates the subject is suitable for treatment with an inhibitor of TL1A activity or expression.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is above a cutoff.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of polymorphisms determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with Response Probability Score (RPS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI above a cutoff indicates the subject is suitable for treatment with an inhibitor of TL1A activity or expression,
wherein the PRI has a positive correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is above a cutoff,
wherein the PRI has a positive correlation coefficient with RPS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of polymorphisms determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with Model Risk Score (MRS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI above a cutoff indicates the subject is suitable for treatment with an inhibitor of TL1A activity or expression,
wherein the PRI has a positive correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is above a cutoff,
wherein the PRI has a positive correlation coefficient with MRS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the PRI above a cutoff indicates the subject is suitable for treatment with an inhibitor of TL1A activity or expression.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression if the PRI is above a cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the PRI above a cutoff indicates the subject is suitable for treatment with an inhibitor of TL1A activity or expression,
wherein the PRI has a positive correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression if the PRI is above a cutoff,
wherein the PRI has a positive correlation coefficient with RPS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the PRI above a cutoff indicates the subject is suitable for treatment with an inhibitor of TL1A activity or expression,
wherein the PRI has a positive correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression if the PRI is above a cutoff,
wherein the PRI has a positive correlation coefficient with MRS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) below a cutoff, wherein the PRI is calculated from a combination of polymorphisms determined from a sample from the subject and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with Response Probability Score (RPS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject, wherein the PRI has a negative correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI below a cutoff indicates the subject is suitable for treatment with an inhibitor of TL1A activity or expression,
wherein the PRI has a negative correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is below a cutoff,
wherein the PRI has a negative correlation coefficient with RPS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) below a cutoff, wherein the PRI is calculated from a combination of polymorphisms determined from a sample from the subject and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with Model Risk Score (MRS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI below a cutoff indicates the subject is suitable for treatment with an inhibitor of TL1A activity or expression,
wherein the PRI has a negative correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is below a cutoff,
wherein the PRI has a negative correlation coefficient with MRS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the PRI below a cutoff indicates the subject is suitable for treatment with an inhibitor of TL1A activity or expression,
wherein the PRI has a negative correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression if the PRI is below a cutoff,
wherein the PRI has a negative correlation coefficient with RPS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the PRI below a cutoff indicates the subject is suitable for treatment with an inhibitor of TL1A activity or expression,
wherein the PRI has a negative correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression if the PRI is below a cutoff,
wherein the PRI has a negative correlation coefficient with MRS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of genotypes determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the PRI above a cutoff indicates the subject is suitable for treatment with an inhibitor of TL1A activity or expression.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression if the PRI is above a cutoff.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of genotypes determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with Response Probability Score (RPS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the PRI above a cutoff indicates the subject is suitable for treatment with an inhibitor of TL1A activity or expression,
wherein the PRI has a positive correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression if the PRI is above a cutoff,
wherein the PRI has a positive correlation coefficient with RPS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) above a cutoff, wherein the PRI is calculated from a combination of genotypes determined from a sample from the subject and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with Model Risk Score (MRS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the PRI above a cutoff indicates the subject is suitable for treatment with an inhibitor of TL1A activity or expression,
wherein the PRI has a positive correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression if the PRI is above a cutoff,
wherein the PRI has a positive correlation coefficient with MRS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the PRI above a cutoff indicates the subject is suitable for treatment with an inhibitor of TL1A activity or expression.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression if the PRI is above a cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the PRI above a cutoff indicates the subject is suitable for treatment with an inhibitor of TL1A activity or expression,
wherein the PRI has a positive correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression if the PRI is above a cutoff,
wherein the PRI has a positive correlation coefficient with RPS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject,
wherein the PRI has a positive correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the PRI above a cutoff indicates the subject is suitable for treatment with an inhibitor of TL1A activity or expression,
wherein the PRI has a positive correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression if the PRI is above a cutoff,
wherein the PRI has a positive correlation coefficient with MRS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) below a cutoff, wherein the PRI is calculated from a combination of genotypes determined from a sample from the subject and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with Response Probability Score (RPS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the PRI below a cutoff indicates the subject is suitable for treatment with an inhibitor of TL1A activity or expression,
wherein the PRI has a negative correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression if the PRI is below a cutoff,
wherein the PRI has a negative correlation coefficient with RPS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a Predictive Response Index (PRI) below a cutoff, wherein the PRI is calculated from a combination of genotypes determined from a sample from the subject and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with Model Risk Score (MRS).

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the PRI below a cutoff indicates the subject is suitable for treatment with an inhibitor of TL1A activity or expression,
wherein the PRI has a negative correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression if the PRI is below a cutoff,
wherein the PRI has a negative correlation coefficient with MRS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with RPS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the PRI below a cutoff indicates the subject is suitable for treatment with an inhibitor of TL1A activity or expression,
wherein the PRI has a negative correlation coefficient with RPS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression if the PRI is below a cutoff,
wherein the PRI has a negative correlation coefficient with RPS.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression if the PRI is below a cutoff; and
(b) treating the subject by administering a therapeutically effective amount of the inhibitor of TL1A activity or expression to the subject,
wherein the PRI has a negative correlation coefficient with MRS.

In a further aspect, provided herein is a method of determining a Predictive Response Index (PRI) for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the PRI below a cutoff indicates the subject is suitable for treatment with an inhibitor of TL1A activity or expression,
wherein the PRI has a negative correlation coefficient with MRS.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression if the PRI is below a cutoff,
wherein the PRI has a negative correlation coefficient with MRS.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a comparison of a Predictive Response Index (PRI) to a cutoff according to (1) or (2) as follows:
(1) if the PRI has a positive correlation with Response Probability Score (RPS), then the subject is selected if the PRI is above the cutoff; or
(2) if the PRI has a negative correlation with RPS, then the subject is selected if the PRI is below the cutoff;
wherein the PRI is calculated from a combination of genotypes determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression based on a comparison of the PRI to a cutoff according to (1) or (2) as follows:
      (1) if the PRI has a positive correlation with RPS, then the subject is determined to be suitable if the PRI is above the cutoff; or
      (2) if the PRI has a negative correlation with RPS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the comparison is determined according to (1) or (2):
   (1) if the PRI has a positive correlation with RPS, then the subject is determined to be suitable for treatment with an inhibitor of TL1A activity or expression if the PRI is above the cutoff; or
   (2) if the PRI has a negative correlation with RPS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression based on a comparison of the PRI to a cutoff according to (1) or (2) as follows:
   (1) if the PRI has a positive correlation with RPS, then selecting the subject if the PRI is above the cutoff; or
   (2) if the PRI has a negative correlation with RPS, then selecting the subject if the PRI is below the cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression based on a comparison of the PRI to a cutoff according to (1) or (2) as follows:
      (1) if the PRI has a positive correlation with RPS, then the subject is determined to be suitable if the PRI is above the cutoff; or
      (2) if the PRI has a negative correlation with RPS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the comparison is determined according to (1) or (2):
   (1) if the PRI has a positive correlation with RPS, then the subject is determined to be suitable for treatment with an inhibitor of TL1A activity or expression if the PRI is above the cutoff; or
   (2) if the PRI has a negative correlation with RPS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression based on a comparison of the PRI to a cutoff according to (1) or (2) as follows:
   (1) if the PRI has a positive correlation with RPS, then selecting the subject if the PRI is above the cutoff; or
   (2) if the PRI has a negative correlation with RPS, then selecting the subject if the PRI is below the cutoff.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a comparison of a Predictive Response Index (PRI) to a cutoff according to (1) or (2) as follows:
(1) if the PRI has a positive correlation with Model Risk Score (MRS), then the subject is selected if the PRI is above the cutoff; or
(2) if the PRI has a negative correlation with MRS, then the subject is selected if the PRI is below the cutoff;
wherein the PRI is calculated from a combination of genotypes determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of genotypes;
   (iii) calculating a Predictive Response Index (PRI) from the combination of genotypes, wherein the subject is determined to be suitable for treatment with an inhibitor of TL1A activity or expression based on a comparison of the PRI to a cutoff according to (1) or (2) as follows:
      (1) if the PRI has a positive correlation with MRS, then the subject is determined to be suitable if the PRI is above the cutoff; or
      (2) if the PRI has a negative correlation with MRS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of genotypes; and
(c) calculating the PRI from the combination of genotypes, wherein the comparison is determined according to (1) or (2):
   (1) if the PRI has a positive correlation with MRS, then the subject is determined to be suitable for treatment with an inhibitor of TL1A activity or expression if the PRI is above the cutoff; or
   (2) if the PRI has a negative correlation with MRS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of genotypes;
(c) calculating a Predictive Response Index (PRI) from the combination of genotypes; and
(d) selecting the subject for treatment with the inhibitor of TL1A activity or expression based on a comparison of the PRI to a cutoff according to (1) or (2) as follows:
   (1) if the PRI has a positive correlation with MRS, then selecting the subject if the PRI is above the cutoff; or
   (2) if the PRI has a negative correlation with MRS, then selecting the subject if the PRI is below the cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of genotypes;
   (iv) calculating a Predictive Response Index (PRI) from the combination of genotypes, wherein the subject is determined to be suitable for treatment with an inhibitor of TL1A activity or expression based on a comparison of the PRI to a cutoff according to (1) or (2) as follows:
      (1) if the PRI has a positive correlation with MRS, then the subject is determined to be suitable if the PRI is above the cutoff; or
      (2) if the PRI has a negative correlation with MRS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of genotypes; and
(d) calculating the PRI from the combination of genotypes, wherein the comparison is determined according to (1) or (2):
   (1) if the PRI has a positive correlation with MRS, then the subject is determined to be suitable for treatment with an inhibitor of TL1A activity or expression if the PRI is above the cutoff; or
   (2) if the PRI has a negative correlation with MRS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of genotypes;
(d) calculating a Predictive Response Index (PRI) from the combination of genotypes; and
(e) selecting the subject for treatment with the inhibitor of TL1A activity or expression based on a comparison of the PRI to a cutoff according to (1) or (2) as follows:
   (1) if the PRI has a positive correlation with MRS, then selecting the subject if the PRI is above the cutoff; or
   (2) if the PRI has a negative correlation with MRS, then selecting the subject if the PRI is below the cutoff.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a comparison of a Predictive Response Index (PRI) to a cutoff according to (1) or (2) as follows:
(1) if the PRI has a positive correlation with Response Probability Score (RPS), then the subject is selected if the PRI is above the cutoff; or
(2) if the PRI has a negative correlation with RPS, then the subject is selected if the PRI is below the cutoff;
wherein the PRI is calculated from a combination of polymorphisms determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2) as follows:
      (1) if the PRI has a positive correlation with RPS, then the subject is determined to be suitable if the PRI is above the cutoff; or
      (2) if the PRI has a negative correlation with RPS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the comparison is determined according to (1) or (2):
   (1) if the PRI has a positive correlation with RPS, then the subject is determined to be suitable for treatment with an inhibitor of TL1A activity or expression if the PRI is above the cutoff; or
   (2) if the PRI has a negative correlation with RPS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression based on a comparison of the PRI to a cutoff according to (1) or (2) as follows:
   (1) if the PRI has a positive correlation with RPS, then selecting the subject if the PRI is above the cutoff; or
   (2) if the PRI has a negative correlation with RPS, then selecting the subject if the PRI is below the cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2) as follows:
      (1) if the PRI has a positive correlation with RPS, then the subject is determined to be suitable if the PRI is above the cutoff; or
      (2) if the PRI has a negative correlation with RPS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the comparison is determined according to (1) or (2):
   (1) if the PRI has a positive correlation with RPS, then the subject is determined to be suitable for treatment with an inhibitor of TL1A activity or expression if the PRI is above the cutoff; or
   (2) if the PRI has a negative correlation with RPS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression based on a comparison of the PRI to a cutoff according to (1) or (2) as follows:
   (1) if the PRI has a positive correlation with RPS, then selecting the subject if the PRI is above the cutoff; or
   (2) if the PRI has a negative correlation with RPS, then selecting the subject if the PRI is below the cutoff.

In one aspect, provided herein is a method of treating an inflammatory, a fibrotic, or a fibrostenotic disease or condition in a subject, the method comprising: administering to the subject a therapeutically effective amount of an inhibitor of tumor necrosis factor-like cytokine 1A (TL1A) activity or expression, wherein the subject is selected based on a comparison of a Predictive Response Index (PRI) to a cutoff according to (1) or (2) as follows:
(1) if the PRI has a positive correlation with Model Risk Score (MRS), then the subject is selected if the PRI is above the cutoff; or
(2) if the PRI has a negative correlation with MRS, then the subject is selected if the PRI is below the cutoff;
wherein the PRI is calculated from a combination of polymorphisms determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2) as follows:
      (1) if the PRI has a positive correlation with MRS, then the subject is determined to be suitable if the PRI is above the cutoff; or
      (2) if the PRI has a negative correlation with MRS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the comparison is determined according to (1) or (2):
   (1) if the PRI has a positive correlation with MRS, then the subject is determined to be suitable for treatment with an inhibitor of TL1A activity or expression if the PRI is above the cutoff; or
   (2) if the PRI has a negative correlation with MRS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression based on a comparison of the PRI to a cutoff according to (1) or (2) as follows:
   (1) if the PRI has a positive correlation with MRS, then selecting the subject if the PRI is above the cutoff; or
   (2) if the PRI has a negative correlation with MRS, then selecting the subject if the PRI is below the cutoff.

In another aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) preparing DNA from the sample;
   (iii) subjecting the DNA to an assay adapted to detect a combination of polymorphisms;
   (iv) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2) as follows:
      (1) if the PRI has a positive correlation with MRS, then the subject is determined to be suitable if the PRI is above the cutoff; or
      (2) if the PRI has a negative correlation with MRS, then the subject is determined to be suitable if the PRI is below the cutoff; and
(b) treating the subject by administering a therapeutically effective amount of the inhibitor of TL1A activity or expression to the subject.

In a further aspect, provided herein is a method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) preparing DNA from the sample;
(c) subjecting the DNA to an assay adapted to detect a combination of polymorphisms; and
(d) calculating the PRI from the combination of polymorphisms, wherein the comparison is determined according to (1) or (2):
   (1) if the PRI has a positive correlation with MRS, then the subject is determined to be suitable for treatment with an inhibitor of TL1A activity or expression if the PRI is above the cutoff; or
   (2) if the PRI has a negative correlation with MRS, then the subject is determined to be suitable for treatment with an inhibitor of TL1A activity or expression if the PRI is below the cutoff.

In yet another aspect, provided herein is a method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) preparing DNA from the sample;
(c) subjecting the DNA to an assay adapted to detect a combination of polymorphisms;
(d) calculating a Predictive Response Index (PRI) from the combination of polymorphisms; and
(e) selecting the subject for treatment with the inhibitor of TL1A activity or expression based on a comparison of the PRI to a cutoff according to (1) or (2) as follows:
   (1) if the PRI has a positive correlation with MRS, then selecting the subject if the PRI is above the cutoff; or
   (2) if the PRI has a negative correlation with MRS, then selecting the subject if the PRI is below the cutoff.

In one aspect, provided herein is a computer-implemented methods comprising: (a) receiving a combination of genotypes of a subject with an inflammatory, a fibrotic, or a fibrostenotic disease or condition; and (b) analyzing the combination of genotypes to determine the subject is responsive to a treatment based on a Predictive Response Index (PRI) above a cutoff, wherein the PRI is calculated from the combination of genotypes and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In one aspect, provided herein is a computer-implemented methods comprising: (a) receiving a combination of polymorphisms of a subject with an inflammatory, a fibrotic, or a fibrostenotic disease or condition; and (b) analyzing the combination of polymorphisms to determine the subject is responsive to a treatment of an inhibitor of TL1A activity or expression based on a Predictive Response Index (PRI) above a cutoff, wherein the PRI is calculated from the combination of polymorphisms and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%.

In one aspect, provided herein is a computer-implemented methods comprising: (a) receiving a combination of genotypes of a subject with an inflammatory, a fibrotic, or a fibrostenotic disease or condition; and (b) analyzing the combination of genotypes to determine the subject is responsive to a treatment based on a Predictive Response Index (PRI) above a cutoff, wherein the PRI is calculated from the combination of genotypes and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with RPS, MRS, or both RPS and MRS.

In one aspect, provided herein is a computer-implemented methods comprising: (a) receiving a combination of polymorphisms of a subject with an inflammatory, a fibrotic, or a fibrostenotic disease or condition; and (b) analyzing the combination of polymorphisms to determine the subject is responsive to a treatment of an inhibitor of TL1A activity or expression based on a Predictive Response Index (PRI) above a cutoff, wherein the PRI is calculated from the combination of polymorphisms and the PRI above the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a positive correlation coefficient with RPS, MRS, or both RPS and MRS.

In one aspect, provided herein is a computer-implemented methods comprising: (a) receiving a combination of genotypes of a subject with an inflammatory, a fibrotic, or a fibrostenotic disease or condition; and (b) analyzing the combination of genotypes to determine the subject is responsive to a treatment based on a Predictive Response Index (PRI) below a cutoff, wherein the PRI is calculated from the combination of genotypes and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with RPS, MRS, or both RPS and MRS.

In one aspect, provided herein is a computer-implemented methods comprising: (a) receiving a combination of polymorphisms of a subject with an inflammatory, a fibrotic, or a fibrostenotic disease or condition; and (b) analyzing the combination of polymorphisms to determine the subject is responsive to a treatment of an inhibitor of TL1A activity or expression based on a Predictive Response Index (PRI) below a cutoff, wherein the PRI is calculated from the combination of polymorphisms and the PRI below the cutoff predicts a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, or 51%, wherein the PRI has a negative correlation coefficient with RPS, MRS, or both RPS and MRS.

In one aspect, provided herein is a computer-implemented method of determining a Response Probability Score (RPS) for a subject, the method comprising:
(a) receiving genotype data obtained from a sample from the subject with an inflammatory, a fibrotic, or a fibrostenotic disease or condition, wherein the genotype data comprises a combination of polymorphisms;
(b) analyzing the genotype data with a first statistical algorithm configured to produce a Model Risk Score (MRS) for the subject by performing operations comprising:
   (i) assigning a weighted numerical value to each polymorphism in the combination of polymorphisms to produce a plurality of weighted values; and
   (ii) summing the plurality of weighted values;
(c) providing the MRS to a second statistical algorithm configured to perform a logarithmic function on the MRS to produce a Response Probability Score (RPS); and
(d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition.

In another aspect, provided herein is a computer-implemented method of determining a Response Probability Score (RPS) for a subject, the method comprising:
(a) obtaining a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
(b) receiving genotype data for a plurality of polymorphisms obtained from a sample from the subject;
(c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
(d) applying a logarithmic scale and a cutoff to the MRS to produce a Response Probability Score (RPS).

In a further aspect, provided herein is a method of treating an inflammatory, a fibrotic, or a fibrostenotic disease or condition in a subject, the method comprising: administering to the subject a therapeutically effective amount of an inhibitor of TL1A activity or expression, based, at least partially, on a Predictive Response Index (PRI) calculated by applying one or more statistical algorithms to a combination of polymorphisms detected from a sample obtained from the subject and determining a comparison of the PRI to a cutoff to predicts a positive therapeutic response in the subject to a treatment with the inhibitor of TL1A activity or expression.

In yet another aspect, provided herein is a method of treating an inflammatory, a fibrotic, or a fibrostenotic disease or condition in a subject, the method comprising: administering an inhibitor of TL1A activity or expression to the subject that is predicted to exhibit a positive therapeutic response to the inhibitor of TL1A activity or expression, as determined by a Predictive Response Index (PRI) that is calculated by:
(a) detecting a presence of a combination of polymorphisms in a sample from the subject;
(b) applying a statistical algorithm to the combination of polymorphisms detected in step (a) to generate the PRI; and
(c) determining a comparison of the PRI to a cutoff.

In one aspect, provided herein is a method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
   (i) obtaining or having obtained a sample from the subject;
   (ii) subjecting the sample to an assay adapted to detect a combination of polymorphisms;
   (iii) calculating a Predictive Response Index (PRI) from the combination of polymorphisms, wherein the PRI is further determined in a comparison to a cutoff; and
(b) treating the subject by administering a therapeutically effective amount of the inhibitor of TL1A activity or expression to the subject.

In various embodiments of the methods provided herein including in Sections 2, 5.2 (e.g. the preceding paragraphs), and 7, the method further comprises preparing DNA from the sample.

In various embodiments of the methods provided herein including in Sections 2, 5.2 (e.g. the preceding paragraphs), and 7, the comparison of PRI to a cutoff is determined according to (1) or (2): (1) if the PRI has a positive correlation with RPS, then determining the PRI of the subject if the PRI is above the cutoff, or (2) if the PRI has a negative correlation with RPS, then determining the PRI of the subject if the PRI is below the cutoff.

The term “model risk score” or “MRS” is calculated as weighted summation of the SNPs in the SNP combination model or

$$MRS = {β}_{0} + {\sum }_{i = 1}^{n} ( {β}_{i} ) \times ( {χ}_{i} ) ,$$

wherein χᵢ is a mathematical representation of the ith SNP in the model, which can be any one of the mathematical representations described in Table 28. In some examples of the methods provided herein, the MRS can be used as the basis for binarily classifying a patient as a responder (CDx positive) or non-responder (CDx negative). The cutoff of the MRS for determining such binary classification of the patient can be determined by training the model with the therapeutic response data from a patient cohort such that the MRS cutoff provide the highest accuracy for classifying the responder, the non-responder, or both the responder and the non-responder, as described elsewhere in this disclosure, for example in Section 7.20.4. Such MRS cutoff can also be determined by other machine learning or computerized clustering methods known and practiced in the art and described elsewhere in this disclosure, for example in Section 7.20.4. In general, if the MRS is ≥cutoff, the prediction is “yes, responder” and CDx positive and if the MRS is <cutoff, the prediction is “no, non-responder” and CDx negative.

The term “Response Probability Score” or “RPS” refers to a score calculated via a mathematical function using the patient's genotype or combination of genotypes as the variable input, wherein genotype input for any SNP in the model can be a mathematical representation selected from the mathematical representations described in Table 28. Such RPS can be used to indicate the probability of the patient having a therapeutic response to the treatment of an TL1A inhibitor, with higher RPS indicating higher probability of the patient having a therapeutic response and lower RPS indicating lower probability of the patient having a therapeutic response. In some examples of the methods provided herein, the RPS can be used as the basis for binarily classifying a patient as a responder (CDx positive) or non-responder (CDx negative). The cutoff of the RPS for determining such binary classification of the patient can be determined by training the model with the therapeutic response data from a patient cohort such that the RPS cutoff provide the highest accuracy for classifying the responder, the non-responder, or both the responder and the non-responder, as described elsewhere in this disclosure, for example in Section 7.20.4. Such RPS cutoff can also be determined by other machine learning or computerized clustering methods known and practiced in the art and as described elsewhere in this disclosure, for example in Section 7.20.4. In other examples, the mathematical function (e.g coefficients in the function) used to calculate the RPS from patient's genotype or combination of genotypes can be trained and normalized such that the RPS has a range of 0 to 1 and 0.5 is cutoff for optimally classifying patient as a responder (CDx positive) or non-responder (CDx negative) to TL1A inhibitor treatment. In one specific example, the RPS is calculated as described in this Section and Section 7.20.4. In another specific example, the RPS is calculated as RPS=1/(1+e^{(-MRS)}) wherein (1) if the RPS is ≥0.5, the prediction is “yes, responder” and CDx positive and (2) if the RPS is <0.5, the prediction is “no, non-responder” and CDx negative. In general, if the RPS is ≥cutoff, the prediction is “yes, responder” and CDx positive and if the RPS is <cutoff, the prediction is “no, non-responder” and CDx negative.

The term “Predictive Response Index” or “PRI” refers to a value calculated via a mathematical function using the patient's genotype or combination of genotypes as the variable input, which value is predictive of (i) an increase in a level of TNFSF15 (TL1A) protein expression in a sample obtained from a subject or patient, as compared to a reference level of TNFSF15 (TL1A) protein expression (e.g., derived from a normal individual), (ii) an increase of IBD enriched cell types as compared to a reference level in a tissue not affected by IBD, (iii) a decrease of IBD depleted cell types as compared to a reference level in a tissue not affected by IBD, and/or (iv) an increase of a positive therapeutic response in IBD patients to a treatment with the TL1A inhibitor as compared to the reference level of response in patients not selected by the genotypes or models, wherein genotype input for any SNP in the model can be a mathematical representation selected from the mathematical representations described in Table 28. Such a PRI can be MRS described in this Section and Section 7. Such a PRI can also be RPS described in this Section and Section 7. PRI can also be a mathematical function that generates scores correlated positively or negatively with MRS or RPS calculated from the subjects' genotypes. In some examples, the PRI correlates with RPS with a correlation coefficient of at least about 0.7, at least about 0.75, at least about 0.8, at least about 0.85, at least about 0.9, at least about 0.95, or at least about 0.99. In other examples, the PRI correlates with RPS with a correlation coefficient of at most about −0.7, at most about −0.75, at most about −0.8, at most about −0.85, at most about −0.9, at most about −0.95, or at most about −0.99.

In some embodiments of the various methods provided herein, including in Sections 2, 5.2, and 7, the MRS is calculated with a combination of β coefficients and the corresponding SNPs, wherein the combination of β coefficients and the corresponding SNPs is selected from the combinations described in columns 1 and 2 of Table 31. In some embodiments of the various methods provided herein, including in Sections 2, 5.2, and 7, the MRS is calculated with a combination of β coefficients and the corresponding SNPs as

$$MRS = {β}_{0} + {\sum }_{i = 1}^{n} ( {β}_{i} ) \times ( {χ}_{i} ) ,$$

wherein the combination of β coefficients and the corresponding SNPs is selected from the combinations described in columns 1 and 2 of Table 31. In some embodiments of the various methods provided herein, including in Sections 2, 5.2, and 7, the MRS is calculated with a combination of β coefficients and the corresponding SNPs as

$$MRS = {β}_{0} + {\sum }_{i = 1}^{n} ( {β}_{i} ) \times ( {χ}_{i} ) ,$$

wherein the combination of β coefficients and the corresponding SNPs is selected from the combinations described in columns 1 and 2 of Table 31, and wherein the χᵢ is a mathematical representation of the ith SNP in the model which can be any one of the mathematical representations described in Table 28. As is clear from the description of Table 31, each row in columns 1 and 2 of Table 31 describes a specific combination of β coefficients and the corresponding SNPs (e.g. the β coefficients of row 3 in column 1 correspond to the SNP encodings of row 3 in column 2). Accordingly, in some embodiments of the various methods provided herein including in this paragraph, including in Sections 2, 5.2, and 7, the MRS is calculated with a combination of β coefficients and the corresponding SNPs, wherein the combination of β coefficients and the corresponding SNPs can be that described in any one row of columns 1 and 2 of Table 31. More specifically, in some embodiments of the various methods provided herein including in this paragraph, the MRS is calculated with a combination of β coefficients and the corresponding SNPs, wherein the combination of β coefficients and the corresponding SNPs is described in row x of columns 1 and 2 of Table 31, and wherein x is any number between 2 to 1374.

In some embodiments of the various methods provided herein, including in Sections 2, 5.2, and 7, the RPS is calculated with a combination of β coefficients and the corresponding SNPs, wherein the combination of β coefficients and the corresponding SNPs is selected from the combinations described in columns 1 and 2 of Table 31. In some embodiments of the various methods provided herein, including in Sections 2, 5.2, and 7, the RPS is calculated with a combination of β coefficients and the corresponding SNPs as RPS=1/(1+e^{(-MRS)}), wherein

$$MRS = {β}_{0} + {\sum }_{i = 1}^{n} ( {β}_{i} ) \times ( {χ}_{i} ) ,$$

and wherein the combination of β coefficients and the corresponding SNPs is selected from the combinations described in columns 1 and 2 of Table 31. In some embodiments of the various methods provided herein, including in Sections 2, 5.2, and 7, the RPS is calculated with a combination of β coefficients and the corresponding SNPs as RPS=1/(1+e^{(-MRS)}), wherein

$$MRS = {β}_{0} + {\sum }_{i = 1}^{n} ( {β}_{i} ) \times ( {χ}_{i} ) ,$$

wherein the combination of β coefficients and the corresponding SNPs is selected from the combinations described in columns 1 and 2 of Table 31, and wherein the χᵢ is a mathematical representation of the ith SNP in the model which can be any one of the mathematical representations described in Table 28. As is clear from the description of Table 31, each row in columns 1 and 2 of Table 31 describes a specific combination of β coefficients and the corresponding SNPs (e.g. the β coefficients of row 3 in column 1 correspond to the combination of SNP encodings of row 3 in column 2). Accordingly, in some embodiments of the various methods provided herein including in this paragraph, the RPS is calculated with a combination of β coefficients and the corresponding SNPs, wherein the combination of β coefficients and the corresponding SNPs can be that described in any one row of columns 1 and 2 of Table 31. More specifically, in some embodiments of the various methods provided herein including in this paragraph, the RPS is calculated with a combination of β coefficients and the corresponding SNPs, wherein the combination of β coefficients and the corresponding SNPs is described in row x of columns 1 and 2 of Table 31, and wherein x is any number between 2 to 1374.

In some embodiments of the various methods provided herein, the PRI positively correlates with RPS with a correlation coefficient of at least about 0.6. In some embodiments of the various methods provided herein, the PRI positively correlates with RPS with a correlation coefficient of at least about 0.65. In some embodiments of the various methods provided herein, the PRI positively correlates with RPS with a correlation coefficient of at least about 0.7. In some embodiments of the various methods provided herein, the PRI positively correlates with RPS with a correlation coefficient of at least about 0.75. In some embodiments of the various methods provided herein, the PRI positively correlates with RPS with a correlation coefficient of at least about 0.8. In some embodiments of the various methods provided herein, the PRI positively correlates with RPS with a correlation coefficient of at least about 0.85. In some embodiments of the various methods provided herein, the PRI positively correlates with RPS with a correlation coefficient of at least about 0.9. In some embodiments of the various methods provided herein, the PRI positively correlates with RPS with a correlation coefficient of at least about 0.95. In some embodiments of the various methods provided herein, the PRI positively correlates with RPS with a correlation coefficient of at least about 0.99.

In some embodiments of the various methods provided herein, the PRI negatively correlates with RPS with a correlation coefficient of at most about −0.6. In some embodiments of the various methods provided herein, the PRI negatively correlates with RPS with a correlation coefficient of at most about −0.65. In some embodiments of the various methods provided herein, the PRI negatively correlates with RPS with a correlation coefficient of at most about −0.7. In some embodiments of the various methods provided herein, the PRI negatively correlates with RPS with a correlation coefficient of at most about −0.75. In some embodiments of the various methods provided herein, the PRI negatively correlates with RPS with a correlation coefficient of at most about −0.8. In some embodiments of the various methods provided herein, the PRI negatively correlates with RPS with a correlation coefficient of at most about −0.85. In some embodiments of the various methods provided herein, the PRI negatively correlates with RPS with a correlation coefficient of at most about −0.9. In some embodiments of the various methods provided herein, the PRI negatively correlates with RPS with a correlation coefficient of at most about −0.95. In some embodiments of the various methods provided herein, the PRI negatively correlates with RPS with a correlation coefficient of at most about −0.99.

In some embodiments of the various methods provided herein, the PRI positively correlates with RPS with a correlation coefficient of about 0.6. In some embodiments of the various methods provided herein, the PRI positively correlates with RPS with a correlation coefficient of about 0.65. In some embodiments of the various methods provided herein, the PRI positively correlates with RPS with a correlation coefficient of about 0.7. In some embodiments of the various methods provided herein, the PRI positively correlates with RPS with a correlation coefficient of about 0.75. In some embodiments of the various methods provided herein, the PRI positively correlates with RPS with a correlation coefficient of about 0.8. In some embodiments of the various methods provided herein, the PRI positively correlates with RPS with a correlation coefficient of about 0.85. In some embodiments of the various methods provided herein, the PRI positively correlates with RPS with a correlation coefficient of about 0.9. In some embodiments of the various methods provided herein, the PRI positively correlates with RPS with a correlation coefficient of about 0.95. In some embodiments of the various methods provided herein, the PRI positively correlates with RPS with a correlation coefficient of about 0.99.

In some embodiments of the various methods provided herein, the PRI negatively correlates with RPS with a correlation coefficient of about −0.6. In some embodiments of the various methods provided herein, the PRI negatively correlates with RPS with a correlation coefficient of about −0.65. In some embodiments of the various methods provided herein, the PRI negatively correlates with RPS with a correlation coefficient of about −0.7. In some embodiments of the various methods provided herein, the PRI negatively correlates with RPS with a correlation coefficient of about −0.75. In some embodiments of the various methods provided herein, the PRI negatively correlates with RPS with a correlation coefficient of about −0.8. In some embodiments of the various methods provided herein, the PRI negatively correlates with RPS with a correlation coefficient of about −0.85. In some embodiments of the various methods provided herein, the PRI negatively correlates with RPS with a correlation coefficient of about −0.9. In some embodiments of the various methods provided herein, the PRI negatively correlates with RPS with a correlation coefficient of about −0.95. In some embodiments of the various methods provided herein, the PRI negatively correlates with RPS with a correlation coefficient of about −0.99.

In some embodiments of the various methods provided herein, the PRI positively correlates with MRS with a correlation coefficient of at least about 0.6. In some embodiments of the various methods provided herein, the PRI positively correlates with MRS with a correlation coefficient of at least about 0.65. In some embodiments of the various methods provided herein, the PRI positively correlates with MRS with a correlation coefficient of at least about 0.7. In some embodiments of the various methods provided herein, the PRI positively correlates with MRS with a correlation coefficient of at least about 0.75. In some embodiments of the various methods provided herein, the PRI positively correlates with MRS with a correlation coefficient of at least about 0.8. In some embodiments of the various methods provided herein, the PRI positively correlates with MRS with a correlation coefficient of at least about 0.85. In some embodiments of the various methods provided herein, the PRI positively correlates with MRS with a correlation coefficient of at least about 0.9. In some embodiments of the various methods provided herein, the PRI positively correlates with MRS with a correlation coefficient of at least about 0.95. In some embodiments of the various methods provided herein, the PRI positively correlates with MRS with a correlation coefficient of at least about 0.99.

In some embodiments of the various methods provided herein, the PRI negatively correlates with MRS with a correlation coefficient of at most about −0.6. In some embodiments of the various methods provided herein, the PRI negatively correlates with MRS with a correlation coefficient of at most about −0.65. In some embodiments of the various methods provided herein, the PRI negatively correlates with MRS with a correlation coefficient of at most about −0.7. In some embodiments of the various methods provided herein, the PRI negatively correlates with MRS with a correlation coefficient of at most about −0.75. In some embodiments of the various methods provided herein, the PRI negatively correlates with MRS with a correlation coefficient of at most about −0.8. In some embodiments of the various methods provided herein, the PRI negatively correlates with MRS with a correlation coefficient of at most about −0.85. In some embodiments of the various methods provided herein, the PRI negatively correlates with MRS with a correlation coefficient of at most about −0.9. In some embodiments of the various methods provided herein, the PRI negatively correlates with MRS with a correlation coefficient of at most about −0.95. In some embodiments of the various methods provided herein, the PRI negatively correlates with MRS with a correlation coefficient of at most about −0.99.

In some embodiments of the various methods provided herein, the PRI positively correlates with MRS with a correlation coefficient of about 0.6. In some embodiments of the various methods provided herein, the PRI positively correlates with MRS with a correlation coefficient of about 0.65. In some embodiments of the various methods provided herein, the PRI positively correlates with MRS with a correlation coefficient of about 0.7. In some embodiments of the various methods provided herein, the PRI positively correlates with MRS with a correlation coefficient of about 0.75. In some embodiments of the various methods provided herein, the PRI positively correlates with MRS with a correlation coefficient of about 0.8. In some embodiments of the various methods provided herein, the PRI positively correlates with MRS with a correlation coefficient of about 0.85. In some embodiments of the various methods provided herein, the PRI positively correlates with MRS with a correlation coefficient of about 0.9. In some embodiments of the various methods provided herein, the PRI positively correlates with MRS with a correlation coefficient of about 0.95. In some embodiments of the various methods provided herein, the PRI positively correlates with MRS with a correlation coefficient of about 0.99.

In some embodiments of the various methods provided herein, the PRI negatively correlates with MRS with a correlation coefficient of about −0.6. In some embodiments of the various methods provided herein, the PRI negatively correlates with MRS with a correlation coefficient of about −0.65. In some embodiments of the various methods provided herein, the PRI negatively correlates with MRS with a correlation coefficient of about −0.7. In some embodiments of the various methods provided herein, the PRI negatively correlates with MRS with a correlation coefficient of about −0.75. In some embodiments of the various methods provided herein, the PRI negatively correlates with MRS with a correlation coefficient of about −0.8. In some embodiments of the various methods provided herein, the PRI negatively correlates with MRS with a correlation coefficient of about −0.85. In some embodiments of the various methods provided herein, the PRI negatively correlates with MRS with a correlation coefficient of about −0.9. In some embodiments of the various methods provided herein, the PRI negatively correlates with MRS with a correlation coefficient of about −0.95. In some embodiments of the various methods provided herein, the PRI negatively correlates with MRS with a correlation coefficient of about −0.99.

In some specific embodiment of the various methods provided herein, including in Sections 2, 5.2, and 7, the PRI is the MRS. In some specific embodiment of the various methods provided herein, including in Sections 2, 5.2, and 7, the PRI is the RPS. In some specific embodiment of the various methods provided herein, including in Sections 2, 5.2, and 7, the PRI equals to the MRS. In some specific embodiment of the various methods provided herein, including in Sections 2, 5.2, and 7, the PRI equals to the RPS. In some specific embodiment of the various methods provided herein, including in Sections 2, 5.2, and 7, the PRI equals to −1×MRS (correlation coefficient of −1 with MRS). In some specific embodiment of the various methods provided herein, including in Sections 2, 5.2, and 7, the PRI equals to −1×RPS (correlation coefficient of −1 with RPS).

In some embodiments of the various methods provided herein, including in Sections 2, 5.2, and 7, the RPS ranges from 0 to 1. In some embodiments, the cutoff for RPS is 0.5.

In some embodiments of the various methods provided herein, including in Sections 2, 5.2, and 7, the β₀ used for the calculation of MRS, RPS, and/or PRI is about 0.0077127943934849. In some embodiments of the various methods provided herein, including in Sections 2, 5.2, and 7, the β₀ used for the calculation of MRS, RPS, and/or PRI is 0.0077127943934849. In some embodiments of the various methods provided herein, including in Sections 2, 5.2, and 7, the β₀ used for the calculation of MRS, RPS, and/or PRI is about 0.008. In some embodiments of the various methods provided herein, including in Sections 2, 5.2, and 7, the cutoff for MRS is about 0.0322446725024791. In some embodiments of the various methods provided herein, including in Sections 2, 5.2, and 7, the cutoff for MRS is 0.0322446725024791. In some embodiments of the various methods provided herein, including in Sections 2, 5.2, and 7, the cutoff for MRS is about 0.03.

In some embodiment of the various methods provided herein, including in Sections 2, 5.2 (e.g. the preceding paragraphs), and 7, the polymorphisms in the model are mathematically represented by χᵢ for calculating PRI, RPS, or MRS as: (i) 0 for homozygous reference alleles, 1 for heterozygous reference and alternative alleles, and 2 for homozygous alternative alleles (additive numeric encoding); (ii) 1 for homozygous reference alleles, 1 for heterozygous reference and alternative alleles, and 0 for homozygous alternative alleles (ref negative numeric encoding); (iii) 0 for homozygous reference alleles, 1 for heterozygous reference and alternative alleles, and 1 for homozygous alternative alleles (alt negative numeric encoding); (iv) 0 for homozygous reference alleles, 0 for heterozygous reference and alternative alleles, and 1 for homozygous alternative alleles (alt recessive numeric encoding); (v) 1 for homozygous reference alleles, 0 for heterozygous reference and alternative alleles, and 0 for homozygous alternative alleles (ref recessive numeric encoding); and/or (vi) 0 for homozygous reference alleles, 1 for heterozygous reference and alternative alleles, and 0 for homozygous alternative alleles (haploinsufficient numeric encoding). In some embodiment of the various methods provided herein, including in Sections 2, 5.2 (e.g. the preceding paragraphs), and 7, the genotypes in the model are mathematically represented by χᵢ for calculating PRI, RPS, or MRS as: (i) 0 for homozygous reference alleles, 1 for heterozygous reference and alternative alleles, and 2 for homozygous alternative alleles; (ii) 1 for homozygous reference alleles, 1 for heterozygous reference and alternative alleles, and 0 for homozygous alternative alleles; (iii) 0 for homozygous reference alleles, 1 for heterozygous reference and alternative alleles, and 1 for homozygous alternative alleles; (iv) 0 for homozygous reference alleles, 0 for heterozygous reference and alternative alleles, and 1 for homozygous alternative alleles; (v) 1 for homozygous reference alleles, 0 for heterozygous reference and alternative alleles, and 0 for homozygous alternative alleles; and/or (vi) 0 for homozygous reference alleles, 1 for heterozygous reference and alternative alleles, and 0 for homozygous alternative alleles.

In certain embodiment of the various methods provided herein, including in Sections 2, 5.2 (e.g. the preceding paragraphs), and 7, each polymorphism is represented by χᵢ for calculating PRI, RPS, or MRS as: (i) 0 for homozygous reference alleles, 1 for heterozygous reference and alternative alleles, and 2 for homozygous alternative alleles; (ii) 1 for homozygous reference alleles, 1 for heterozygous reference and alternative alleles, and 0 for homozygous alternative alleles; (iii) 0 for homozygous reference alleles, 1 for heterozygous reference and alternative alleles, and 1 for homozygous alternative alleles; (iv) 0 for homozygous reference alleles, 0 for heterozygous reference and alternative alleles, and 1 for homozygous alternative alleles; (v) 1 for homozygous reference alleles, 0 for heterozygous reference and alternative alleles, and 0 for homozygous alternative alleles; and/or (vi) 0 for homozygous reference alleles, 1 for heterozygous reference and alternative alleles, and 0 for homozygous alternative alleles. Accordingly, for the various methods provided herein, including in Sections 2, 5.2 (e.g. the preceding paragraphs), and 7, each polymorphism can be represented by any one of the 6 numeric encodings described in Table 28. In some embodiments of the various methods provided herein, including in Sections 2, 5.2 (e.g. the preceding paragraphs), and 7, each polymorphism can be represented by any two of the 6 numeric encodings described in Table 28. In some embodiments of the various methods provided herein, including in Sections 2, 5.2 (e.g. the preceding paragraphs), and 7, each polymorphism can be represented by any three of the 6 numeric encodings described in Table 28. In some embodiments of the various methods provided herein, including in Sections 2, 5.2 (e.g. the preceding paragraphs), and 7, each polymorphism can be represented by any four of the 6 numeric encodings described in Table 28. In some embodiments of the various methods provided herein, including in Sections 2, 5.2 (e.g. the preceding paragraphs), and 7, each polymorphism can be represented by any five of the 6 numeric encodings described in Table 28. In some embodiments of the various methods provided herein, including in Sections 2, 5.2 (e.g. the preceding paragraphs), and 7, each polymorphism can be represented by all six of the 6 numeric encodings described in Table 28. Additionally, for the various methods provided herein, including in Sections 2, 5.2 (e.g. the preceding paragraphs), and 7, any one or more or all polymorphisms can be represented by any one of the 6 numeric encodings described in Table 28. In some embodiments of the various methods provided herein, including in Sections 2, 5.2 (e.g. the preceding paragraphs), and 7, any one or more or all polymorphisms can be represented by any two of the 6 numeric encodings described in Table 28. In some embodiments of the various methods provided herein, including in Sections 2, 5.2 (e.g. the preceding paragraphs), and 7, any one or more or all polymorphisms can be represented by any three of the 6 numeric encodings described in Table 28. In some embodiments of the various methods provided herein, including in Sections 2, 5.2 (e.g. the preceding paragraphs), and 7, any one or more or all polymorphisms can be represented by any four of the 6 numeric encodings described in Table 28. In some embodiments of the various methods provided herein, including in Sections 2, 5.2 (e.g. the preceding paragraphs), and 7, any one or more or all polymorphisms can be represented by any five of the 6 numeric encodings described in Table 28. In some embodiments of the various methods provided herein, including in Sections 2, 5.2 (e.g. the preceding paragraphs), and 7, any one or more or all polymorphisms can be represented by all six of the 6 numeric encodings described in Table 28. In some embodiments of the various methods provided herein, including in Sections 2, 5.2 (e.g. the preceding paragraphs), and 7, any one or more or all polymorphisms can be represented by any 1, 2, 3, 4, 5, or 6 of the 6 numeric encodings described in Table 28. Consequently the combination of polymorphisms can be represented by any combination of the 6 numeric encodings described in Table 28. For example, a 3-SNP CDx model may have each of the 3 SNPs represented by one encoding selected from Table 28; each of the 3 SNPs represented by two encodings selected from Table 28; each of the 3 SNPs represented by three encodings selected from Table 28; each of the 3 SNPs represented by four encodings selected from Table 28; each of the 3 SNPs represented by five encodings selected from Table 28; each of the 3 SNPs represented by six encodings selected from Table 28; 1 of the 3 SNPs represented by 1 of the encodings and 2 of the 3 SNPs represented by two encodings selected from Table 28; etc. Table 31 describes some of the specific embodiments of the CDx models based on SNP combinations and the representation of the SNPs in the model.

In certain embodiment of the various methods provided herein, including in Sections 2, 5.2 (e.g. the preceding paragraphs), and 7, each genotype is represented by χᵢ for calculating PRI, RPS, or MRS as: (i) 0 for homozygous reference alleles, 1 for heterozygous reference and alternative alleles, and 2 for homozygous alternative alleles; (ii) 1 for homozygous reference alleles, 1 for heterozygous reference and alternative alleles, and 0 for homozygous alternative alleles; (iii) 0 for homozygous reference alleles, 1 for heterozygous reference and alternative alleles, and 1 for homozygous alternative alleles; (iv) 0 for homozygous reference alleles, 0 for heterozygous reference and alternative alleles, and 1 for homozygous alternative alleles; (v) 1 for homozygous reference alleles, 0 for heterozygous reference and alternative alleles, and 0 for homozygous alternative alleles; and/or (vi) 0 for homozygous reference alleles, 1 for heterozygous reference and alternative alleles, and 0 for homozygous alternative alleles. Accordingly, for the various methods provided herein, including in Sections 2, 5.2 (e.g. the preceding paragraphs), and 7, each genotype can be represented by any one of the 6 numeric encodings described in Table 28. In some embodiments of the various methods provided herein, including in Sections 2, 5.2 (e.g. the preceding paragraphs), and 7, each genotype can be represented by any two of the 6 numeric encodings described in Table 28. In some embodiments of the various methods provided herein, including in Sections 2, 5.2 (e.g. the preceding paragraphs), and 7, each genotype can be represented by any three of the 6 numeric encodings described in Table 28. In some embodiments of the various methods provided herein, including in Sections 2, 5.2 (e.g. the preceding paragraphs), and 7, each genotype can be represented by any four of the 6 numeric encodings described in Table 28. In some embodiments of the various methods provided herein, including in Sections 2, 5.2 (e.g. the preceding paragraphs), and 7, each genotype can be represented by any five of the 6 numeric encodings described in Table 28. In some embodiments of the various methods provided herein, including in Sections 2, 5.2 (e.g. the preceding paragraphs), and 7, each genotype can be represented by all six of the 6 numeric encodings described in Table 28. Additionally, for the various methods provided herein, including in Sections 2, 5.2 (e.g. the preceding paragraphs), and 7, any one or more or all genotypes can be represented by any one of the 6 numeric encodings described in Table 28. In some embodiments of the various methods provided herein, including in Sections 2, 5.2 (e.g. the preceding paragraphs), and 7, any one or more or all genotypes can be represented by any two of the 6 numeric encodings described in Table 28. In some embodiments of the various methods provided herein, including in Sections 2, 5.2 (e.g. the preceding paragraphs), and 7, any one or more or all genotypes can be represented by any three of the 6 numeric encodings described in Table 28. In some embodiments of the various methods provided herein, including in Sections 2, 5.2 (e.g. the preceding paragraphs), and 7, any one or more or all genotypes can be represented by any four of the 6 numeric encodings described in Table 28. In some embodiments of the various methods provided herein, including in Sections 2, 5.2 (e.g. the preceding paragraphs), and 7, any one or more or all genotypes can be represented by any five of the 6 numeric encodings described in Table 28. In some embodiments of the various methods provided herein, including in Sections 2, 5.2 (e.g. the preceding paragraphs), and 7, any one or more or all genotypes can be represented by all six of the 6 numeric encodings described in Table 28. In some embodiments of the various methods provided herein, including in Sections 2, 5.2 (e.g. the preceding paragraphs), and 7, any one or more or all genotypes can be represented by any 1, 2, 3, 4, 5, or 6 of the 6 numeric encodings described in Table 28. Consequently the combination of genotypes can be represented by any combination of the 6 numeric encodings described in Table 28. For example, a 3-SNP CDx model may have each of the 3 SNPs represented by one encoding selected from Table 28; each of the 3 SNPs represented by two encodings selected from Table 28; each of the 3 SNPs represented by three encodings selected from Table 28; each of the 3 SNPs represented by four encodings selected from Table 28; each of the 3 SNPs represented by five encodings selected from Table 28; each of the 3 SNPs represented by six encodings selected from Table 28; 1 of the 3 SNPs represented by 1 of the encodings and 2 of the 3 SNPs represented by two encodings selected from Table 28; etc. Table 31 describes some of the specific embodiments of the CDx models based on SNP combinations and the representation of the SNPs in the model.

In various embodiments of the methods provided herein including in Sections 2, 5.2 (e.g. the preceding paragraphs), and 7, the methods comprise interpreting the genotype, combination of genotypes, the polymorphism, or the combination of polymorphisms by calculating a PRI, MRS, or RPS. In some embodiments, the interpreting step is performed by an interpretation software. In certain embodiments, the interpretation software is as described in Sections 5.8.1, 5.8.2, 5.8.3, 5.8.4, 1.1.1, and 5.8.5. In various embodiments of the methods provided herein including in Sections 2, 5.2 (e.g. the preceding paragraphs), and 7, the methods comprise interpreting the genotype, combination of genotypes, the polymorphism, or the combination of polymorphisms with an interpretation software. In certain embodiments, the step of interpreting the genotype, combination of genotypes, the polymorphism, or the combination of polymorphisms is any embodiment of such interpreting step as described in Sections 5.8.1, 5.8.2, 5.8.3, 5.8.4, 1.1.1, and 5.8.5. In some embodiments, the step of interpreting the genotype, combination of genotypes, the polymorphism, or the combination of polymorphisms is performed with the software as described in Sections 5.8.1, 5.8.2, 5.8.3, 5.8.4, 1.1.1, and 5.8.5. In some embodiments, the step of interpreting the genotype, combination of genotypes, the polymorphism, or the combination of polymorphisms is performed in a system as described in Sections 5.8.1, 5.8.2, 5.8.3, 5.8.4, 1.1.1, and 5.8.5. In some embodiments, the step of interpreting the genotype, combination of genotypes, the polymorphism, or the combination of polymorphisms is performed with a software as described in Sections 5.8.1, 5.8.2, 5.8.3, 5.8.4, 1.1.1, and 5.8.5 and in a system as described in Sections 5.8.1, 5.8.2, 5.8.3, 5.8.4, 1.1.1, and 5.8.5.

In various embodiments of the methods provided herein including in Sections 2, 5.2 (e.g. the preceding paragraphs), and 7, the combination of polymorphisms comprises a combination of genotypes. In various embodiments of the methods provided herein including in Sections 2, 5.2 (e.g. the preceding paragraphs), and 7, the combination of polymorphisms consists of a combination of genotypes.

In various embodiments of the methods provided herein including in Sections 2, 5.2 (e.g. the preceding paragraphs), and 7, the combination of polymorphisms comprises or consists of 2 polymorphisms. In some embodiments, the combination of polymorphisms comprises or consists of 3 polymorphisms. In certain embodiments, the combination of polymorphisms comprises or consists of 4 polymorphisms. In some other embodiments, the combination of polymorphisms comprises or consists of 5 polymorphisms. In some additional embodiments, the combination of polymorphisms comprises or consists of 6 polymorphisms. In some embodiments, the combination of polymorphisms comprises or consists of 7 polymorphisms. In certain embodiments, the combination of polymorphisms comprises or consists of 8 polymorphisms. In some other embodiments, the combination of polymorphisms comprises or consists of 9 polymorphisms. In some additional embodiments, the combination of polymorphisms comprises or consists of 10 polymorphisms. In various embodiments of the methods provided herein including in Sections 2, 5.2 (e.g. the preceding paragraphs), and 7, the combination of polymorphisms comprises or consists of at least 2 polymorphisms. In some embodiments, the combination of polymorphisms comprises or consists of at least 3 polymorphisms. In certain embodiments, the combination of polymorphisms comprises or consists of at least 4 polymorphisms. In some other embodiments, the combination of polymorphisms comprises or consists of at least 5 polymorphisms. In some additional embodiments, the combination of polymorphisms comprises or consists of at least 6 polymorphisms. In some embodiments, the combination of polymorphisms comprises or consists of at least 7 polymorphisms. In certain embodiments, the combination of polymorphisms comprises or consists of at least 8 polymorphisms. In some other embodiments, the combination of polymorphisms comprises or consists of at least 9 polymorphisms. In some additional embodiments, the combination of polymorphisms comprises or consists of at least 10 polymorphisms.

In various embodiments of the methods provided herein including in Sections 2, 5.2 (e.g. the preceding paragraphs), and 7, the method further comprises preparing DNA from the sample obtained from the subject.

In various embodiments of the methods provided herein including in Sections 2, 5.2 (e.g. the preceding paragraphs), and 7, the method further comprises contacting the sample with one or more probes adapted to detect the presence of the combination of genotype or the combination of polymorphisms. In various embodiments of the methods provided herein including in Sections 2, 5.2 (e.g. the preceding paragraphs), and 7, the method further comprises contacting the sample with an assay adapted to detect the presence of the combination of genotype or the combination of polymorphisms.

In various embodiments of the methods provided herein including in Sections 2, 5.2 (e.g. the preceding paragraphs), and 7, the method further comprises processing the sample to enrich the genetic materials. In various embodiments of the methods provided herein including in Sections 2, 5.2 (e.g. the preceding paragraphs), and 7, the method further comprises processing the sample to enrich the genetic materials targeted by the assay or the one or more probes.

In various embodiments of the methods provided herein including in Sections 2, 5.2 (e.g. the preceding paragraphs), and 7, the PRI above the cutoff predicts a positive therapeutic response in the subject to a treatment with the inhibitor of TL1A activity or expression with a positive predictive value of at least 25%. The other embodiments described in this paragraph are provided for the various methods described herein including in Sections 2, 5.2 (e.g. the preceding paragraphs), and 7. In some embodiments, the PRI above the cutoff predicts a positive therapeutic response in the subject to a treatment with the inhibitor of TL1A activity or expression with a positive predictive value of at least 26%. In some embodiments, the PRI above the cutoff predicts a positive therapeutic response in the subject to a treatment with the inhibitor of TL1A activity or expression with a positive predictive value of at least 27%. In some embodiments, the PRI above the cutoff predicts a positive therapeutic response in the subject to a treatment with the inhibitor of TL1A activity or expression with a positive predictive value of at least 28%. In some embodiments, the PRI above the cutoff predicts a positive therapeutic response in the subject to a treatment with the inhibitor of TL1A activity or expression with a positive predictive value of at least 29%. In some embodiments, the PRI above the cutoff predicts a positive therapeutic response in the subject to a treatment with the inhibitor of TL1A activity or expression with a positive predictive value of at least 30%. In some embodiments, the PRI above the cutoff predicts a positive therapeutic response in the subject to a treatment with the inhibitor of TL1A activity or expression with a positive predictive value of at least 31%. In some embodiments, the PRI above the cutoff predicts a positive therapeutic response in the subject to a treatment with the inhibitor of TL1A activity or expression with a positive predictive value of at least 32%. In some embodiments, the PRI above the cutoff predicts a positive therapeutic response in the subject to a treatment with the inhibitor of TL1A activity or expression with a positive predictive value of at least 33%. In some embodiments, the PRI above the cutoff predicts a positive therapeutic response in the subject to a treatment with the inhibitor of TL1A activity or expression with a positive predictive value of at least 34%. In some embodiments, the PRI above the cutoff predicts a positive therapeutic response in the subject to a treatment with the inhibitor of TL1A activity or expression with a positive predictive value of at least 35%. In some embodiments, the PRI above the cutoff predicts a positive therapeutic response in the subject to a treatment with the inhibitor of TL1A activity or expression with a positive predictive value of at least 40%. In some embodiments, the PRI above the cutoff predicts a positive therapeutic response in the subject to a treatment with the inhibitor of TL1A activity or expression with a positive predictive value of at least 45%. In some embodiments, the PRI above the cutoff predicts a positive therapeutic response in the subject to a treatment with the inhibitor of TL1A activity or expression with a positive predictive value of at least 50%. In some embodiments, the PRI above the cutoff predicts a positive therapeutic response in the subject to a treatment with the inhibitor of TL1A activity or expression with a positive predictive value of at least 51%. In some embodiments, the PRI above the cutoff predicts a positive therapeutic response in the subject to a treatment with the inhibitor of TL1A activity or expression with a positive predictive value of at least 55%. In some embodiments, the PRI above the cutoff predicts a positive therapeutic response in the subject to a treatment with the inhibitor of TL1A activity or expression with a positive predictive value of at least 60%. In some embodiments, the PRI above the cutoff predicts a positive therapeutic response in the subject to a treatment with the inhibitor of TL1A activity or expression with a positive predictive value of at least 65%. In some embodiments, the PRI above the cutoff predicts a positive therapeutic response in the subject to a treatment with the inhibitor of TL1A activity or expression with a positive predictive value of at least 70%. In some embodiments, the PRI above the cutoff predicts a positive therapeutic response in the subject to a treatment with the inhibitor of TL1A activity or expression with a positive predictive value of at least 75%. In some embodiments, the PRI above the cutoff predicts a positive therapeutic response in the subject to a treatment with the inhibitor of TL1A activity or expression with a positive predictive value of at least 80%. In some embodiments, the PRI above the cutoff predicts a positive therapeutic response in the subject to a treatment with the inhibitor of TL1A activity or expression with a positive predictive value of at least 85%. In some embodiments, the PRI above the cutoff predicts a positive therapeutic response in the subject to a treatment with the inhibitor of TL1A activity or expression with a positive predictive value of at least 90%. In some embodiments, the PRI above the cutoff predicts a positive therapeutic response in the subject to a treatment with the inhibitor of TL1A activity or expression with a positive predictive value of at least 95%. In some embodiments, the PRI above the cutoff predicts a positive therapeutic response in the subject to a treatment with the inhibitor of TL1A activity or expression with a positive predictive value of about 25%. In some embodiments, the PRI above the cutoff predicts a positive therapeutic response in the subject to a treatment with the inhibitor of TL1A activity or expression with a positive predictive value of about 26%. In some embodiments, the PRI above the cutoff predicts a positive therapeutic response in the subject to a treatment with the inhibitor of TL1A activity or expression with a positive predictive value of about 27%. In some embodiments, the PRI above the cutoff predicts a positive therapeutic response in the subject to a treatment with the inhibitor of TL1A activity or expression with a positive predictive value of about 28%. In some embodiments, the PRI above the cutoff predicts a positive therapeutic response in the subject to a treatment with the inhibitor of TL1A activity or expression with a positive predictive value of about 29%. In some embodiments, the PRI above the cutoff predicts a positive therapeutic response in the subject to a treatment with the inhibitor of TL1A activity or expression with a positive predictive value of about 30%. In some embodiments, the PRI above the cutoff predicts a positive therapeutic response in the subject to a treatment with the inhibitor of TL1A activity or expression with a positive predictive value of about 31%. In some embodiments, the PRI above the cutoff predicts a positive therapeutic response in the subject to a treatment with the inhibitor of TL1A activity or expression with a positive predictive value of about 32%. In some embodiments, the PRI above the cutoff predicts a positive therapeutic response in the subject to a treatment with the inhibitor of TL1A activity or expression with a positive predictive value of about 33%. In some embodiments, the PRI above the cutoff predicts a positive therapeutic response in the subject to a treatment with the inhibitor of TL1A activity or expression with a positive predictive value of about 34%. In some embodiments, the PRI above the cutoff predicts a positive therapeutic response in the subject to a treatment with the inhibitor of TL1A activity or expression with a positive predictive value of about 35%. In some embodiments, the PRI above the cutoff predicts a positive therapeutic response in the subject to a treatment with the inhibitor of TL1A activity or expression with a positive predictive value of about 40%. In some embodiments, the PRI above the cutoff predicts a positive therapeutic response in the subject to a treatment with the inhibitor of TL1A activity or expression with a positive predictive value of about 45%. In some embodiments, the PRI above the cutoff predicts a positive therapeutic response in the subject to a treatment with the inhibitor of TL1A activity or expression with a positive predictive value of about 50%. In some embodiments, the PRI above the cutoff predicts a positive therapeutic response in the subject to a treatment with the inhibitor of TL1A activity or expression with a positive predictive value of about 51%. In some embodiments, the PRI above the cutoff predicts a positive therapeutic response in the subject to a treatment with the inhibitor of TL1A activity or expression with a positive predictive value of about 55%. In some embodiments, the PRI above the cutoff predicts a positive therapeutic response in the subject to a treatment with the inhibitor of TL1A activity or expression with a positive predictive value of about 60%. In some embodiments, the PRI above the cutoff predicts a positive therapeutic response in the subject to a treatment with the inhibitor of TL1A activity or expression with a positive predictive value of about 65%. In some embodiments, the PRI above the cutoff predicts a positive therapeutic response in the subject to a treatment with the inhibitor of TL1A activity or expression with a positive predictive value of about 70%. In some embodiments, the PRI above the cutoff predicts a positive therapeutic response in the subject to a treatment with the inhibitor of TL1A activity or expression with a positive predictive value of about 75%. In some embodiments, the PRI above the cutoff predicts a positive therapeutic response in the subject to a treatment with the inhibitor of TL1A activity or expression with a positive predictive value of about 80%. In some embodiments, the PRI above the cutoff predicts a positive therapeutic response in the subject to a treatment with the inhibitor of TL1A activity or expression with a positive predictive value of about 85%. In some embodiments, the PRI above the cutoff predicts a positive therapeutic response in the subject to a treatment with the inhibitor of TL1A activity or expression with a positive predictive value of about 90%. In some embodiments, the PRI above the cutoff predicts a positive therapeutic response in the subject to a treatment with the inhibitor of TL1A activity or expression with a positive predictive value of about 95%. In some embodiments, the PRI above the cutoff predicts a positive therapeutic response in the subject to a treatment with the inhibitor of TL1A activity or expression with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a positive therapeutic response in the subject to a treatment with the inhibitor of TL1A activity or expression with a negative predictive value of at least 40%. The other embodiments described in this paragraph are provided for the various methods described herein including in Sections 2, 5.2 (e.g. the preceding paragraphs), and 7. In some embodiments, the PRI above the cutoff predicts a positive therapeutic response in the subject to a treatment with the inhibitor of TL1A activity or expression with a negative predictive value of at least 45%. In some embodiments, the PRI above the cutoff predicts a positive therapeutic response in the subject to a treatment with the inhibitor of TL1A activity or expression with a negative predictive value of at least 50%. In some embodiments, the PRI above the cutoff predicts a positive therapeutic response in the subject to a treatment with the inhibitor of TL1A activity or expression with a negative predictive value of at least 55%. In some embodiments, the PRI above the cutoff predicts a positive therapeutic response in the subject to a treatment with the inhibitor of TL1A activity or expression with a negative predictive value of at least 60%. In some embodiments, the PRI above the cutoff predicts a positive therapeutic response in the subject to a treatment with the inhibitor of TL1A activity or expression with a negative predictive value of at least 65%. In some embodiments, the PRI above the cutoff predicts a positive therapeutic response in the subject to a treatment with the inhibitor of TL1A activity or expression with a negative predictive value of at least 70%. In some embodiments, the PRI above the cutoff predicts a positive therapeutic response in the subject to a treatment with the inhibitor of TL1A activity or expression with a negative predictive value of at least 75%. In some embodiments, the PRI above the cutoff predicts a positive therapeutic response in the subject to a treatment with the inhibitor of TL1A activity or expression with a negative predictive value of at least 80%. In some embodiments, the PRI above the cutoff predicts a positive therapeutic response in the subject to a treatment with the inhibitor of TL1A activity or expression with a negative predictive value of at least 85%. In some embodiments, the PRI above the cutoff predicts a positive therapeutic response in the subject to a treatment with the inhibitor of TL1A activity or expression with a negative predictive value of at least 90%. In some embodiments, the PRI above the cutoff predicts a positive therapeutic response in the subject to a treatment with the inhibitor of TL1A activity or expression with a negative predictive value of at least 95%. In some embodiments, the PRI above the cutoff predicts a positive therapeutic response in the subject to a treatment with the inhibitor of TL1A activity or expression with a negative predictive value of about 40%. In some embodiments, the PRI above the cutoff predicts a positive therapeutic response in the subject to a treatment with the inhibitor of TL1A activity or expression with a negative predictive value of about 45%. In some embodiments, the PRI above the cutoff predicts a positive therapeutic response in the subject to a treatment with the inhibitor of TL1A activity or expression with a negative predictive value of about 50%. In some embodiments, the PRI above the cutoff predicts a positive therapeutic response in the subject to a treatment with the inhibitor of TL1A activity or expression with a negative predictive value of about 55%. In some embodiments, the PRI above the cutoff predicts a positive therapeutic response in the subject to a treatment with the inhibitor of TL1A activity or expression with a negative predictive value of about 60%. In some embodiments, the PRI above the cutoff predicts a positive therapeutic response in the subject to a treatment with the inhibitor of TL1A activity or expression with a negative predictive value of about 65%. In some embodiments, the PRI above the cutoff predicts a positive therapeutic response in the subject to a treatment with the inhibitor of TL1A activity or expression with a negative predictive value of about 70%. In some embodiments, the PRI above the cutoff predicts a positive therapeutic response in the subject to a treatment with the inhibitor of TL1A activity or expression with a negative predictive value of about 75%. In some embodiments, the PRI above the cutoff predicts a positive therapeutic response in the subject to a treatment with the inhibitor of TL1A activity or expression with a negative predictive value of about 80%. In some embodiments, the PRI above the cutoff predicts a positive therapeutic response in the subject to a treatment with the inhibitor of TL1A activity or expression with a negative predictive value of about 85%. In some embodiments, the PRI above the cutoff predicts a positive therapeutic response in the subject to a treatment with the inhibitor of TL1A activity or expression with a negative predictive value of about 90%. In some embodiments, the PRI above the cutoff predicts a positive therapeutic response in the subject to a treatment with the inhibitor of TL1A activity or expression with a negative predictive value of about 95%. In some embodiments, the PRI above the cutoff predicts a positive therapeutic response in the subject to a treatment with the inhibitor of TL1A activity or expression with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a positive therapeutic response in the subject to a treatment with the inhibitor of TL1A activity or expression with a sensitivity of at least 20%. The other embodiments described in this paragraph are provided for the various methods described herein including in Sections 2, 5.2 (e.g. the preceding paragraphs), and 7. In some embodiments, the PRI above the cutoff predicts a positive therapeutic response in the subject to a treatment with the inhibitor of TL1A activity or expression with a sensitivity of at least 25%. In some embodiments, the PRI above the cutoff predicts a positive therapeutic response in the subject to a treatment with the inhibitor of TL1A activity or expression with a sensitivity of at least 30%. In some embodiments, the PRI above the cutoff predicts a positive therapeutic response in the subject to a treatment with the inhibitor of TL1A activity or expression with a sensitivity of at least 35%. In some embodiments, the PRI above the cutoff predicts a positive therapeutic response in the subject to a treatment with the inhibitor of TL1A activity or expression with a sensitivity of at least 40%. In some embodiments, the PRI above the cutoff predicts a positive therapeutic response in the subject to a treatment with the inhibitor of TL1A activity or expression with a sensitivity of at least 45%. In some embodiments, the PRI above the cutoff predicts a positive therapeutic response in the subject to a treatment with the inhibitor of TL1A activity or expression with a sensitivity of at least 50%. In some embodiments, the PRI above the cutoff predicts a positive therapeutic response in the subject to a treatment with the inhibitor of TL1A activity or expression with a sensitivity of at least 55%. In some embodiments, the PRI above the cutoff predicts a positive therapeutic response in the subject to a treatment with the inhibitor of TL1A activity or expression with a sensitivity of at least 60%. In some embodiments, the PRI above the cutoff predicts a positive therapeutic response in the subject to a treatment with the inhibitor of TL1A activity or expression with a sensitivity of at least 65%. In some embodiments, the PRI above the cutoff predicts a positive therapeutic response in the subject to a treatment with the inhibitor of TL1A activity or expression with a sensitivity of at least 70%. In some embodiments, the PRI above the cutoff predicts a positive therapeutic response in the subject to a treatment with the inhibitor of TL1A activity or expression with a sensitivity of at least 75%. In some embodiments, the PRI above the cutoff predicts a positive therapeutic response in the subject to a treatment with the inhibitor of TL1A activity or expression with a sensitivity of at least 80%. In some embodiments, the PRI above the cutoff predicts a positive therapeutic response in the subject to a treatment with the inhibitor of TL1A activity or expression with a sensitivity of at least 85%. In some embodiments, the PRI above the cutoff predicts a positive therapeutic response in the subject to a treatment with the inhibitor of TL1A activity or expression with a sensitivity of at least 90%. In some embodiments, the PRI above the cutoff predicts a positive therapeutic response in the subject to a treatment with the inhibitor of TL1A activity or expression with a sensitivity of at least 95%. In some embodiments, the PRI above the cutoff predicts a positive therapeutic response in the subject to a treatment with the inhibitor of TL1A activity or expression with a sensitivity of about 20%. In some embodiments, the PRI above the cutoff predicts a positive therapeutic response in the subject to a treatment with the inhibitor of TL1A activity or expression with a sensitivity of about 25%. In some embodiments, the PRI above the cutoff predicts a positive therapeutic response in the subject to a treatment with the inhibitor of TL1A activity or expression with a sensitivity of about 30%. In some embodiments, the PRI above the cutoff predicts a positive therapeutic response in the subject to a treatment with the inhibitor of TL1A activity or expression with a sensitivity of about 35%. In some embodiments, the PRI above the cutoff predicts a positive therapeutic response in the subject to a treatment with the inhibitor of TL1A activity or expression with a sensitivity of about 40%. In some embodiments, the PRI above the cutoff predicts a positive therapeutic response in the subject to a treatment with the inhibitor of TL1A activity or expression with a sensitivity of about 45%. In some embodiments, the PRI above the cutoff predicts a positive therapeutic response in the subject to a treatment with the inhibitor of TL1A activity or expression with a sensitivity of about 50%. In some embodiments, the PRI above the cutoff predicts a positive therapeutic response in the subject to a treatment with the inhibitor of TL1A activity or expression with a sensitivity of about 55%. In some embodiments, the PRI above the cutoff predicts a positive therapeutic response in the subject to a treatment with the inhibitor of TL1A activity or expression with a sensitivity of about 60%. In some embodiments, the PRI above the cutoff predicts a positive therapeutic response in the subject to a treatment with the inhibitor of TL1A activity or expression with a sensitivity of about 65%. In some embodiments, the PRI above the cutoff predicts a positive therapeutic response in the subject to a treatment with the inhibitor of TL1A activity or expression with a sensitivity of about 70%. In some embodiments, the PRI above the cutoff predicts a positive therapeutic response in the subject to a treatment with the inhibitor of TL1A activity or expression with a sensitivity of about 75%. In some embodiments, the PRI above the cutoff predicts a positive therapeutic response in the subject to a treatment with the inhibitor of TL1A activity or expression with a sensitivity of about 80%. In some embodiments, the PRI above the cutoff predicts a positive therapeutic response in the subject to a treatment with the inhibitor of TL1A activity or expression with a sensitivity of about 85%. In some embodiments, the PRI above the cutoff predicts a positive therapeutic response in the subject to a treatment with the inhibitor of TL1A activity or expression with a sensitivity of about 90%. In some embodiments, the PRI above the cutoff predicts a positive therapeutic response in the subject to a treatment with the inhibitor of TL1A activity or expression with a sensitivity of about 95%. In some embodiments, the PRI above the cutoff predicts a positive therapeutic response in the subject to a treatment with the inhibitor of TL1A activity or expression with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a positive therapeutic response in the subject to a treatment with the inhibitor of TL1A activity or expression with a specificity of at least 40%. The other embodiments described in this paragraph are provided for the various methods described herein including in Sections 2, 5.2 (e.g. the preceding paragraphs), and 7. In some embodiments, the PRI above the cutoff predicts a positive therapeutic response in the subject to a treatment with the inhibitor of TL1A activity or expression with a specificity of at least 45%. In some embodiments, the PRI above the cutoff predicts a positive therapeutic response in the subject to a treatment with the inhibitor of TL1A activity or expression with a specificity of at least 50%. In some embodiments, the PRI above the cutoff predicts a positive therapeutic response in the subject to a treatment with the inhibitor of TL1A activity or expression with a specificity of at least 55%. In some embodiments, the PRI above the cutoff predicts a positive therapeutic response in the subject to a treatment with the inhibitor of TL1A activity or expression with a specificity of at least 60%. In some embodiments, the PRI above the cutoff predicts a positive therapeutic response in the subject to a treatment with the inhibitor of TL1A activity or expression with a specificity of at least 65%. In some embodiments, the PRI above the cutoff predicts a positive therapeutic response in the subject to a treatment with the inhibitor of TL1A activity or expression with a specificity of at least 70%. In some embodiments, the PRI above the cutoff predicts a positive therapeutic response in the subject to a treatment with the inhibitor of TL1A activity or expression with a specificity of at least 75%. In some embodiments, the PRI above the cutoff predicts a positive therapeutic response in the subject to a treatment with the inhibitor of TL1A activity or expression with a specificity of at least 80%. In some embodiments, the PRI above the cutoff predicts a positive therapeutic response in the subject to a treatment with the inhibitor of TL1A activity or expression with a specificity of at least 85%. In some embodiments, the PRI above the cutoff predicts a positive therapeutic response in the subject to a treatment with the inhibitor of TL1A activity or expression with a specificity of at least 90%. In some embodiments, the PRI above the cutoff predicts a positive therapeutic response in the subject to a treatment with the inhibitor of TL1A activity or expression with a specificity of at least 95%. In some embodiments, the PRI above the cutoff predicts a positive therapeutic response in the subject to a treatment with the inhibitor of TL1A activity or expression with a specificity of about 40%. In some embodiments, the PRI above the cutoff predicts a positive therapeutic response in the subject to a treatment with the inhibitor of TL1A activity or expression with a specificity of about 45%. In some embodiments, the PRI above the cutoff predicts a positive therapeutic response in the subject to a treatment with the inhibitor of TL1A activity or expression with a specificity of about 50%. In some embodiments, the PRI above the cutoff predicts a positive therapeutic response in the subject to a treatment with the inhibitor of TL1A activity or expression with a specificity of about 55%. In some embodiments, the PRI above the cutoff predicts a positive therapeutic response in the subject to a treatment with the inhibitor of TL1A activity or expression with a specificity of about 60%. In some embodiments, the PRI above the cutoff predicts a positive therapeutic response in the subject to a treatment with the inhibitor of TL1A activity or expression with a specificity of about 65%. In some embodiments, the PRI above the cutoff predicts a positive therapeutic response in the subject to a treatment with the inhibitor of TL1A activity or expression with a specificity of about 70%. In some embodiments, the PRI above the cutoff predicts a positive therapeutic response in the subject to a treatment with the inhibitor of TL1A activity or expression with a specificity of about 75%. In some embodiments, the PRI above the cutoff predicts a positive therapeutic response in the subject to a treatment with the inhibitor of TL1A activity or expression with a specificity of about 80%. In some embodiments, the PRI above the cutoff predicts a positive therapeutic response in the subject to a treatment with the inhibitor of TL1A activity or expression with a specificity of about 85%. In some embodiments, the PRI above the cutoff predicts a positive therapeutic response in the subject to a treatment with the inhibitor of TL1A activity or expression with a specificity of about 90%. In some embodiments, the PRI above the cutoff predicts a positive therapeutic response in the subject to a treatment with the inhibitor of TL1A activity or expression with a specificity of about 95%. In some embodiments, the PRI above the cutoff predicts a positive therapeutic response in the subject to a treatment with the inhibitor of TL1A activity or expression with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the positive rate of the subjects having a PRI above the cutoff as provided in the methods is at least 10%. The other embodiments described in this paragraph are provided for the various methods described herein including in Sections 2, 5.2 (e.g. the preceding paragraphs), and 7. In some embodiments, the positive rate of the subjects having a PRI above the cutoff as provided in the methods is at least 15%. In some embodiments, the positive rate of the subjects having a PRI above the cutoff as provided in the methods is at least 20%. In some embodiments, the positive rate of the subjects having a PRI above the cutoff as provided in the methods is at least 25%. In some embodiments, the positive rate of the subjects having a PRI above the cutoff as provided in the methods is at least 30%. In some embodiments, the positive rate of the subjects having a PRI above the cutoff as provided in the methods is at least 35%. In some embodiments, the positive rate of the subjects having a PRI above the cutoff as provided in the methods is at least 40%. In some embodiments, the positive rate of the subjects having a PRI above the cutoff as provided in the methods is at least 45%. In some embodiments, the positive rate of the subjects having a PRI above the cutoff as provided in the methods is at least 50%. In some embodiments, the positive rate of the subjects having a PRI above the cutoff as provided in the methods is at least 55%. In some embodiments, the positive rate of the subjects having a PRI above the cutoff as provided in the methods is at least 60%. In some embodiments, the positive rate of the subjects having a PRI above the cutoff as provided in the methods is at least 65%. In some embodiments, the positive rate of the subjects having a PRI above the cutoff as provided in the methods is at least 70%. In some embodiments, the positive rate of the subjects having a PRI above the cutoff as provided in the methods is at least 75%. In some embodiments, the positive rate of the subjects having a PRI above the cutoff as provided in the methods is about 10%. In some embodiments, the positive rate of the subjects having a PRI above the cutoff as provided in the methods is about 15%. In some embodiments, the positive rate of the subjects having a PRI above the cutoff as provided in the methods is about 20%. In some embodiments, the positive rate of the subjects having a PRI above the cutoff as provided in the methods is about 25%. In some embodiments, the positive rate of the subjects having a PRI above the cutoff as provided in the methods is about 30%. In some embodiments, the positive rate of the subjects having a PRI above the cutoff as provided in the methods is about 35%. In some embodiments, the positive rate of the subjects having a PRI above the cutoff as provided in the methods is about 40%. In some embodiments, the positive rate of the subjects having a PRI above the cutoff as provided in the methods is about 45%. In some embodiments, the positive rate of the subjects having a PRI above the cutoff as provided in the methods is about 50%. In some embodiments, the positive rate of the subjects having a PRI above the cutoff as provided in the methods is about 55%. In some embodiments, the positive rate of the subjects having a PRI above the cutoff as provided in the methods is about 60%. In some embodiments, the positive rate of the subjects having a PRI above the cutoff as provided in the methods is about 65%. In some embodiments, the positive rate of the subjects having a PRI above the cutoff as provided in the methods is about 70%. In some embodiments, the positive rate of the subjects having a PRI above the cutoff as provided in the methods is about 75%. In some embodiments, the positive rate of the subjects having a PRI above the cutoff as provided in the methods is from 10% to 75%, from 15% to 75%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, 10% to 70%, from 15% to 70%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 10% to 65%, from 15% to 65%, from 20% to 65%, from 25% to 65%, from 30% to 65%, from 35% to 65%, from 40% to 65%, from 45% to 65%, from 50% to 65%, from 55% to 65%, from 60% to 65%, from 10% to 60%, from 15% to 60%, from 20% to 60%, from 25% to 60%, from 30% to 60%, from 35% to 60%, from 40% to 60%, from 45% to 60%, from 50% to 60%, from 55% to 60%, from 10% to 55%, from 15% to 55%, from 20% to 55%, from 25% to 55%, from 30% to 55%, from 35% to 55%, from 40% to 55%, from 45% to 55%, from 50% to 55%, from 10% to 50%, from 15% to 50%, from 20% to 50%, from 25% to 50%, from 30% to 50%, from 35% to 50%, from 40% to 50%, from 45% to 50%, from 10% to 45%, from 15% to 45%, from 20% to 45%, from 25% to 45%, from 30% to 45%, from 35% to 45%, from 40% to 45%, from 10% to 40%, from 15% to 40%, from 20% to 40%, from 25% to 40%, from 30% to 40%, from 35% to 40%, from 10% to 35%, from 15% to 35%, from 20% to 35%, from 25% to 35%, from 30% to 35%, from 10% to 30%, from 15% to 30%, from 20% to 30%, from 25% to 30%, from 10% to 25%, from 15% to 25%, from 20% to 25%, from 10% to 20%, from 15% to 20%, or from 10% to 15%. In some specific embodiments, the positive rate of the subjects having a PRI above the cutoff as provided in the methods is from 30% to 40%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

As used herein, the term “positive rate,” when used in reference to a matter (e.g. in reference to subjects having PRI above a cutoff or in reference to PRI above a cutoff), means the percentage of the subjects or patients that are positive or determined to be positive for the matter (e.g. positive for having PRI above a cutoff) over the whole subject or patient population. For another example, the positive rate of samples classified as having a PRI above a cutoff means the percentage of samples classified as having a PRI above a cutoff over the population of samples subjected to the classification. The term “negative rate,” when used in reference to a matter (e.g. in reference to subjects having PRI above a cutoff or in reference to PRI above a cutoff), means the percentage of the subjects or patients that are negative or determined to be negative for the matter (e.g. negative for having PRI above a cutoff) over the whole subject or patient population. As a subject or a patient is either positive or negative for having a PRI above a cutoff, the disclosure provides the corresponding embodiments for the negative rate of having PRI above a cutoff, which negative rate is calculated as 100% minus the positive rate of having PRI above the cutoff.

In various embodiments of the methods provided herein including in Sections 2, 5.2 (e.g. the preceding paragraphs), and 7, the PRI above the cutoff predicts a positive therapeutic response in the subject to a treatment with the inhibitor of TL1A activity or expression with an accuracy of at least 50%. The other embodiments described in this paragraph are provided for the various methods described herein including in Sections 2, 5.2 (e.g. the preceding paragraphs), and 7. In some embodiments, the PRI above the cutoff predicts a positive therapeutic response in the subject to a treatment with the inhibitor of TL1A activity or expression with an accuracy of at least 55%. In some embodiments, the PRI above the cutoff predicts a positive therapeutic response in the subject to a treatment with the inhibitor of TL1A activity or expression with an accuracy of at least 60%. In some embodiments, the PRI above the cutoff predicts a positive therapeutic response in the subject to a treatment with the inhibitor of TL1A activity or expression with an accuracy of at least 65%. In some embodiments, the PRI above the cutoff predicts a positive therapeutic response in the subject to a treatment with the inhibitor of TL1A activity or expression with an accuracy of at least 70%. In some embodiments, the PRI above the cutoff predicts a positive therapeutic response in the subject to a treatment with the inhibitor of TL1A activity or expression with an accuracy of at least 75%. In some embodiments, the PRI above the cutoff predicts a positive therapeutic response in the subject to a treatment with the inhibitor of TL1A activity or expression with an accuracy of at least 80%. In some embodiments, the PRI above the cutoff predicts a positive therapeutic response in the subject to a treatment with the inhibitor of TL1A activity or expression with an accuracy of at least 85%. In some embodiments, the PRI above the cutoff predicts a positive therapeutic response in the subject to a treatment with the inhibitor of TL1A activity or expression with an accuracy of at least 90%. In some embodiments, the PRI above the cutoff predicts a positive therapeutic response in the subject to a treatment with the inhibitor of TL1A activity or expression with an accuracy of at least 95%. In some embodiments, the PRI above the cutoff predicts a positive therapeutic response in the subject to a treatment with the inhibitor of TL1A activity or expression with an accuracy of about 50%. In some embodiments, the PRI above the cutoff predicts a positive therapeutic response in the subject to a treatment with the inhibitor of TL1A activity or expression with an accuracy of about 55%. In some embodiments, the PRI above the cutoff predicts a positive therapeutic response in the subject to a treatment with the inhibitor of TL1A activity or expression with an accuracy of about 60%. In some embodiments, the PRI above the cutoff predicts a positive therapeutic response in the subject to a treatment with the inhibitor of TL1A activity or expression with an accuracy of about 65%. In some embodiments, the PRI above the cutoff predicts a positive therapeutic response in the subject to a treatment with the inhibitor of TL1A activity or expression with an accuracy of about 70%. In some embodiments, the PRI above the cutoff predicts a positive therapeutic response in the subject to a treatment with the inhibitor of TL1A activity or expression with an accuracy of about 75%. In some embodiments, the PRI above the cutoff predicts a positive therapeutic response in the subject to a treatment with the inhibitor of TL1A activity or expression with an accuracy of about 80%. In some embodiments, the PRI above the cutoff predicts a positive therapeutic response in the subject to a treatment with the inhibitor of TL1A activity or expression with an accuracy of about 85%. In some embodiments, the PRI above the cutoff predicts a positive therapeutic response in the subject to a treatment with the inhibitor of TL1A activity or expression with an accuracy of about 90%. In some embodiments, the PRI above the cutoff predicts a positive therapeutic response in the subject to a treatment with the inhibitor of TL1A activity or expression with an accuracy of about 95%. In some embodiments, the PRI above the cutoff predicts a positive therapeutic response in the subject to a treatment with the inhibitor of TL1A activity or expression with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

The term “accuracy,” when used in reference to a test for selecting patients for treatment or in the context of a performing metrics of a test for selecting patients for treatment, means the summation of the performance for correctly calling the true positive and correctly calling the true negatives. Accuracy can be calculated as (true positive+true negative)/(true positive+true negative+false positive+false negative)). Accuracy can also be calculated as the balanced accuracy, which is the geometric mean of recall, or sensitivity, across all labels or treatment response phenotypes.

In various embodiments of the methods provided herein including in Sections 2, 5.2 (e.g. the preceding paragraphs), and 7, the PRI above the cutoff predicts an increase of IBD enriched cell types in an IBD-affected tissue as compared to a reference level in a tissue not affected by IBD with a positive predictive value of at least 25%. The other embodiments described in this paragraph are provided for the various methods described herein including in Sections 2, 5.2 (e.g. the preceding paragraphs), and 7. In some embodiments, the PRI above the cutoff predicts an increase of IBD enriched cell types in an TBD-affected tissue as compared to a reference level in a tissue not affected by IBD with a positive predictive value of at least 26%. In some embodiments, the PRI above the cutoff predicts an increase of IBD enriched cell types in an IBD-affected tissue as compared to a reference level in a tissue not affected by IBD with a positive predictive value of at least 27%. In some embodiments, the PRI above the cutoff predicts an increase of IBD enriched cell types in an IBD-affected tissue as compared to a reference level in a tissue not affected by IBD with a positive predictive value of at least 28%. In some embodiments, the PRI above the cutoff predicts an increase of IBD enriched cell types in an IBD-affected tissue as compared to a reference level in a tissue not affected by IBD with a positive predictive value of at least 29%. In some embodiments, the PRI above the cutoff predicts an increase of IBD enriched cell types in an IBD-affected tissue as compared to a reference level in a tissue not affected by IBD with a positive predictive value of at least 30%. In some embodiments, the PRI above the cutoff predicts an increase of IBD enriched cell types in an IBD-affected tissue as compared to a reference level in a tissue not affected by IBD with a positive predictive value of at least 31%. In some embodiments, the PRI above the cutoff predicts an increase of IBD enriched cell types in an IBD-affected tissue as compared to a reference level in a tissue not affected by IBD with a positive predictive value of at least 32%. In some embodiments, the PRI above the cutoff predicts an increase of IBD enriched cell types in an TBD-affected tissue as compared to a reference level in a tissue not affected by IBD with a positive predictive value of at least 33%. In some embodiments, the PRI above the cutoff predicts an increase of IBD enriched cell types in an IBD-affected tissue as compared to a reference level in a tissue not affected by IBD with a positive predictive value of at least 34%. In some embodiments, the PRI above the cutoff predicts an increase of IBD enriched cell types in an IBD-affected tissue as compared to a reference level in a tissue not affected by IBD with a positive predictive value of at least 35%. In some embodiments, the PRI above the cutoff predicts an increase of IBD enriched cell types in an IBD-affected tissue as compared to a reference level in a tissue not affected by IBD with a positive predictive value of at least 40%. In some embodiments, the PRI above the cutoff predicts an increase of IBD enriched cell types in an IBD-affected tissue as compared to a reference level in a tissue not affected by IBD with a positive predictive value of at least 45%. In some embodiments, the PRI above the cutoff predicts an increase of IBD enriched cell types in an IBD-affected tissue as compared to a reference level in a tissue not affected by IBD with a positive predictive value of at least 50%. In some embodiments, the PRI above the cutoff predicts an increase of IBD enriched cell types in an IBD-affected tissue as compared to a reference level in a tissue not affected by IBD with a positive predictive value of at least 51%. In some embodiments, the PRI above the cutoff predicts an increase of IBD enriched cell types in an IBD-affected tissue as compared to a reference level in a tissue not affected by IBD with a positive predictive value of at least 55%. In some embodiments, the PRI above the cutoff predicts an increase of IBD enriched cell types in an IBD-affected tissue as compared to a reference level in a tissue not affected by IBD with a positive predictive value of at least 60%. In some embodiments, the PRI above the cutoff predicts an increase of IBD enriched cell types in an IBD-affected tissue as compared to a reference level in a tissue not affected by IBD with a positive predictive value of at least 65%. In some embodiments, the PRI above the cutoff predicts an increase of IBD enriched cell types in an IBD-affected tissue as compared to a reference level in a tissue not affected by IBD with a positive predictive value of at least 70%. In some embodiments, the PRI above the cutoff predicts an increase of IBD enriched cell types in an IBD-affected tissue as compared to a reference level in a tissue not affected by IBD with a positive predictive value of at least 75%. In some embodiments, the PRI above the cutoff predicts an increase of IBD enriched cell types in an IBD-affected tissue as compared to a reference level in a tissue not affected by IBD with a positive predictive value of at least 80%. In some embodiments, the PRI above the cutoff predicts an increase of IBD enriched cell types in an IBD-affected tissue as compared to a reference level in a tissue not affected by IBD with a positive predictive value of at least 85%. In some embodiments, the PRI above the cutoff predicts an increase of IBD enriched cell types in an IBD-affected tissue as compared to a reference level in a tissue not affected by IBD with a positive predictive value of at least 90%. In some embodiments, the PRI above the cutoff predicts an increase of IBD enriched cell types in an IBD-affected tissue as compared to a reference level in a tissue not affected by IBD with a positive predictive value of at least 95%. In some embodiments, the PRI above the cutoff predicts an increase of IBD enriched cell types in an IBD-affected tissue as compared to a reference level in a tissue not affected by IBD with a positive predictive value of about 25%. In some embodiments, the PRI above the cutoff predicts an increase of IBD enriched cell types in an IBD-affected tissue as compared to a reference level in a tissue not affected by IBD with a positive predictive value of about 26%. In some embodiments, the PRI above the cutoff predicts an increase of IBD enriched cell types in an IBD-affected tissue as compared to a reference level in a tissue not affected by IBD with a positive predictive value of about 27%. In some embodiments, the PRI above the cutoff predicts an increase of IBD enriched cell types in an IBD-affected tissue as compared to a reference level in a tissue not affected by IBD with a positive predictive value of about 28%. In some embodiments, the PRI above the cutoff predicts an increase of IBD enriched cell types in an IBD-affected tissue as compared to a reference level in a tissue not affected by IBD with a positive predictive value of about 29%. In some embodiments, the PRI above the cutoff predicts an increase of IBD enriched cell types in an IBD-affected tissue as compared to a reference level in a tissue not affected by IBD with a positive predictive value of about 30%. In some embodiments, the PRI above the cutoff predicts an increase of IBD enriched cell types in an IBD-affected tissue as compared to a reference level in a tissue not affected by IBD with a positive predictive value of about 31%. In some embodiments, the PRI above the cutoff predicts an increase of IBD enriched cell types in an IBD-affected tissue as compared to a reference level in a tissue not affected by IBD with a positive predictive value of about 32%. In some embodiments, the PRI above the cutoff predicts an increase of IBD enriched cell types in an IBD-affected tissue as compared to a reference level in a tissue not affected by IBD with a positive predictive value of about 33%. In some embodiments, the PRI above the cutoff predicts an increase of IBD enriched cell types in an IBD-affected tissue as compared to a reference level in a tissue not affected by IBD with a positive predictive value of about 34%. In some embodiments, the PRI above the cutoff predicts an increase of IBD enriched cell types in an IBD-affected tissue as compared to a reference level in a tissue not affected by IBD with a positive predictive value of about 35%. In some embodiments, the PRI above the cutoff predicts an increase of IBD enriched cell types in an IBD-affected tissue as compared to a reference level in a tissue not affected by IBD with a positive predictive value of about 40%. In some embodiments, the PRI above the cutoff predicts an increase of IBD enriched cell types in an IBD-affected tissue as compared to a reference level in a tissue not affected by IBD with a positive predictive value of about 45%. In some embodiments, the PRI above the cutoff predicts an increase of IBD enriched cell types in an IBD-affected tissue as compared to a reference level in a tissue not affected by IBD with a positive predictive value of about 50%. In some embodiments, the PRI above the cutoff predicts an increase of IBD enriched cell types in an IBD-affected tissue as compared to a reference level in a tissue not affected by IBD with a positive predictive value of about 51%. In some embodiments, the PRI above the cutoff predicts an increase of IBD enriched cell types in an IBD-affected tissue as compared to a reference level in a tissue not affected by IBD with a positive predictive value of about 55%. In some embodiments, the PRI above the cutoff predicts an increase of IBD enriched cell types in an IBD-affected tissue as compared to a reference level in a tissue not affected by IBD with a positive predictive value of about 60%. In some embodiments, the PRI above the cutoff predicts an increase of IBD enriched cell types in an IBD-affected tissue as compared to a reference level in a tissue not affected by IBD with a positive predictive value of about 65%. In some embodiments, the PRI above the cutoff predicts an increase of IBD enriched cell types in an IBD-affected tissue as compared to a reference level in a tissue not affected by IBD with a positive predictive value of about 70%. In some embodiments, the PRI above the cutoff predicts an increase of IBD enriched cell types in an IBD-affected tissue as compared to a reference level in a tissue not affected by IBD with a positive predictive value of about 75%. In some embodiments, the PRI above the cutoff predicts an increase of IBD enriched cell types in an IBD-affected tissue as compared to a reference level in a tissue not affected by IBD with a positive predictive value of about 80%. In some embodiments, the PRI above the cutoff predicts an increase of IBD enriched cell types in an IBD-affected tissue as compared to a reference level in a tissue not affected by IBD with a positive predictive value of about 85%. In some embodiments, the PRI above the cutoff predicts an increase of IBD enriched cell types in an IBD-affected tissue as compared to a reference level in a tissue not affected by IBD with a positive predictive value of about 90%. In some embodiments, the PRI above the cutoff predicts an increase of IBD enriched cell types in an IBD-affected tissue as compared to a reference level in a tissue not affected by IBD with a positive predictive value of about 95%. In some embodiments, the PRI above the cutoff predicts an increase of IBD enriched cell types in an IBD-affected tissue as compared to a reference level in a tissue not affected by IBD with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase of IBD enriched cell types in an IBD-affected tissue as compared to a reference level in a tissue not affected by IBD with a negative predictive value of at least 40%. The other embodiments described in this paragraph are provided for the various methods described herein including in Sections 2, 5.2 (e.g. the preceding paragraphs), and 7. In some embodiments, the PRI above the cutoff predicts an increase of IBD enriched cell types in an TBD-affected tissue as compared to a reference level in a tissue not affected by IBD with a negative predictive value of at least 45%. In some embodiments, the PRI above the cutoff predicts an increase of IBD enriched cell types in an IBD-affected tissue as compared to a reference level in a tissue not affected by IBD with a negative predictive value of at least 50%. In some embodiments, the PRI above the cutoff predicts an increase of IBD enriched cell types in an IBD-affected tissue as compared to a reference level in a tissue not affected by IBD with a negative predictive value of at least 55%. In some embodiments, the PRI above the cutoff predicts an increase of IBD enriched cell types in an IBD-affected tissue as compared to a reference level in a tissue not affected by IBD with a negative predictive value of at least 60%. In some embodiments, the PRI above the cutoff predicts an increase of IBD enriched cell types in an IBD-affected tissue as compared to a reference level in a tissue not affected by IBD with a negative predictive value of at least 65%. In some embodiments, the PRI above the cutoff predicts an increase of IBD enriched cell types in an IBD-affected tissue as compared to a reference level in a tissue not affected by IBD with a negative predictive value of at least 70%. In some embodiments, the PRI above the cutoff predicts an increase of IBD enriched cell types in an IBD-affected tissue as compared to a reference level in a tissue not affected by IBD with a negative predictive value of at least 75%. In some embodiments, the PRI above the cutoff predicts an increase of IBD enriched cell types in an IBD-affected tissue as compared to a reference level in a tissue not affected by IBD with a negative predictive value of at least 80%. In some embodiments, the PRI above the cutoff predicts an increase of IBD enriched cell types in an IBD-affected tissue as compared to a reference level in a tissue not affected by IBD with a negative predictive value of at least 85%. In some embodiments, the PRI above the cutoff predicts an increase of IBD enriched cell types in an IBD-affected tissue as compared to a reference level in a tissue not affected by IBD with a negative predictive value of at least 90%. In some embodiments, the PRI above the cutoff predicts an increase of IBD enriched cell types in an IBD-affected tissue as compared to a reference level in a tissue not affected by IBD with a negative predictive value of at least 95%. In some embodiments, the PRI above the cutoff predicts an increase of IBD enriched cell types in an IBD-affected tissue as compared to a reference level in a tissue not affected by IBD with a negative predictive value of about 40%. In some embodiments, the PRI above the cutoff predicts an increase of IBD enriched cell types in an IBD-affected tissue as compared to a reference level in a tissue not affected by IBD with a negative predictive value of about 45%. In some embodiments, the PRI above the cutoff predicts an increase of IBD enriched cell types in an IBD-affected tissue as compared to a reference level in a tissue not affected by IBD with a negative predictive value of about 50%. In some embodiments, the PRI above the cutoff predicts an increase of IBD enriched cell types in an IBD-affected tissue as compared to a reference level in a tissue not affected by IBD with a negative predictive value of about 55%. In some embodiments, the PRI above the cutoff predicts an increase of IBD enriched cell types in an IBD-affected tissue as compared to a reference level in a tissue not affected by IBD with a negative predictive value of about 60%. In some embodiments, the PRI above the cutoff predicts an increase of IBD enriched cell types in an IBD-affected tissue as compared to a reference level in a tissue not affected by IBD with a negative predictive value of about 65%. In some embodiments, the PRI above the cutoff predicts an increase of IBD enriched cell types in an IBD-affected tissue as compared to a reference level in a tissue not affected by IBD with a negative predictive value of about 70%. In some embodiments, the PRI above the cutoff predicts an increase of IBD enriched cell types in an IBD-affected tissue as compared to a reference level in a tissue not affected by IBD with a negative predictive value of about 75%. In some embodiments, the PRI above the cutoff predicts an increase of IBD enriched cell types in an IBD-affected tissue as compared to a reference level in a tissue not affected by IBD with a negative predictive value of about 80%. In some embodiments, the PRI above the cutoff predicts an increase of IBD enriched cell types in an IBD-affected tissue as compared to a reference level in a tissue not affected by IBD with a negative predictive value of about 85%. In some embodiments, the PRI above the cutoff predicts an increase of IBD enriched cell types in an IBD-affected tissue as compared to a reference level in a tissue not affected by IBD with a negative predictive value of about 90%. In some embodiments, the PRI above the cutoff predicts an increase of IBD enriched cell types in an IBD-affected tissue as compared to a reference level in a tissue not affected by IBD with a negative predictive value of about 95%. In some embodiments, the PRI above the cutoff predicts an increase of IBD enriched cell types in an IBD-affected tissue as compared to a reference level in a tissue not affected by IBD with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase of IBD enriched cell types in an IBD-affected tissue as compared to a reference level in a tissue not affected by IBD with a sensitivity of at least 20%. The other embodiments described in this paragraph are provided for the various methods described herein including in Sections 2, 5.2 (e.g. the preceding paragraphs), and 7. In some embodiments, the PRI above the cutoff predicts an increase of IBD enriched cell types in an IBD-affected tissue as compared to a reference level in a tissue not affected by IBD with a sensitivity of at least 25%. In some embodiments, the PRI above the cutoff predicts an increase of IBD enriched cell types in an IBD-affected tissue as compared to a reference level in a tissue not affected by IBD with a sensitivity of at least 30%. In some embodiments, the PRI above the cutoff predicts an increase of IBD enriched cell types in an IBD-affected tissue as compared to a reference level in a tissue not affected by IBD with a sensitivity of at least 35%. In some embodiments, the PRI above the cutoff predicts an increase of IBD enriched cell types in an IBD-affected tissue as compared to a reference level in a tissue not affected by IBD with a sensitivity of at least 40%. In some embodiments, the PRI above the cutoff predicts an increase of IBD enriched cell types in an IBD-affected tissue as compared to a reference level in a tissue not affected by IBD with a sensitivity of at least 45%. In some embodiments, the PRI above the cutoff predicts an increase of IBD enriched cell types in an IBD-affected tissue as compared to a reference level in a tissue not affected by IBD with a sensitivity of at least 50%. In some embodiments, the PRI above the cutoff predicts an increase of IBD enriched cell types in an IBD-affected tissue as compared to a reference level in a tissue not affected by IBD with a sensitivity of at least 55%. In some embodiments, the PRI above the cutoff predicts an increase of BD enriched cell types in an MID-affected tissue as compared to a reference level in a tissue not affected by IBD with a sensitivity of at least 60%. In some embodiments, the PRI above the cutoff predicts an increase of IBD enriched cell types in an IBD-affected tissue as compared to a reference level in a tissue not affected by IBD with a sensitivity of at least 65%. In some embodiments, the PRI above the cutoff predicts an increase of IBD enriched cell types in an IBD-affected tissue as compared to a reference level in a tissue not affected by IBD with a sensitivity of at least 70%. In some embodiments, the PRI above the cutoff predicts an increase of IBD enriched cell types in an IBD-affected tissue as compared to a reference level in a tissue not affected by IBD with a sensitivity of at least 75%. In some embodiments, the PRI above the cutoff predicts an increase of IBD enriched cell types in an IBD-affected tissue as compared to a reference level in a tissue not affected by IBD with a sensitivity of at least 80%. In some embodiments, the PRI above the cutoff predicts an increase of IBD enriched cell types in an IBD-affected tissue as compared to a reference level in a tissue not affected by IBD with a sensitivity of at least 85%. In some embodiments, the PRI above the cutoff predicts an increase of IBD enriched cell types in an IBD-affected tissue as compared to a reference level in a tissue not affected by IBD with a sensitivity of at least 90%. In some embodiments, the PRI above the cutoff predicts an increase of IBD enriched cell types in an IBD-affected tissue as compared to a reference level in a tissue not affected by IBD with a sensitivity of at least 95%. In some embodiments, the PRI above the cutoff predicts an increase of IBD enriched cell types in an IBD-affected tissue as compared to a reference level in a tissue not affected by IBD with a sensitivity of about 20%. In some embodiments, the PRI above the cutoff predicts an increase of IBD enriched cell types in an TBD-affected tissue as compared to a reference level in a tissue not affected by IBD with a sensitivity of about 25%. In some embodiments, the PRI above the cutoff predicts an increase of IBD enriched cell types in an IBD-affected tissue as compared to a reference level in a tissue not affected by IBD with a sensitivity of about 30%. In some embodiments, the PRI above the cutoff predicts an increase of IBD enriched cell types in an IBD-affected tissue as compared to a reference level in a tissue not affected by IBD with a sensitivity of about 35%. In some embodiments, the PRI above the cutoff predicts an increase of IBD enriched cell types in an IBD-affected tissue as compared to a reference level in a tissue not affected by IBD with a sensitivity of about 40%. In some embodiments, the PRI above the cutoff predicts an increase of IBD enriched cell types in an IBD-affected tissue as compared to a reference level in a tissue not affected by IBD with a sensitivity of about 45%. In some embodiments, the PRI above the cutoff predicts an increase of IBD enriched cell types in an IBD-affected tissue as compared to a reference level in a tissue not affected by IBD with a sensitivity of about 50%. In some embodiments, the PRI above the cutoff predicts an increase of IBD enriched cell types in an IBD-affected tissue as compared to a reference level in a tissue not affected by IBD with a sensitivity of about 55%. In some embodiments, the PRI above the cutoff predicts an increase of IBD enriched cell types in an IBD-affected tissue as compared to a reference level in a tissue not affected by IBD with a sensitivity of about 60%. In some embodiments, the PRI above the cutoff predicts an increase of IBD enriched cell types in an IBD-affected tissue as compared to a reference level in a tissue not affected by IBD with a sensitivity of about 65%. In some embodiments, the PRI above the cutoff predicts an increase of IBD enriched cell types in an IBD-affected tissue as compared to a reference level in a tissue not affected by IBD with a sensitivity of about 70%. In some embodiments, the PRI above the cutoff predicts an increase of IBD enriched cell types in an IBD-affected tissue as compared to a reference level in a tissue not affected by IBD with a sensitivity of about 75%. In some embodiments, the PRI above the cutoff predicts an increase of IBD enriched cell types in an IBD-affected tissue as compared to a reference level in a tissue not affected by IBD with a sensitivity of about 80%. In some embodiments, the PRI above the cutoff predicts an increase of IBD enriched cell types in an IBD-affected tissue as compared to a reference level in a tissue not affected by IBD with a sensitivity of about 85%. In some embodiments, the PRI above the cutoff predicts an increase of IBD enriched cell types in an IBD-affected tissue as compared to a reference level in a tissue not affected by IBD with a sensitivity of about 90%. In some embodiments, the PRI above the cutoff predicts an increase of IBD enriched cell types in an IBD-affected tissue as compared to a reference level in a tissue not affected by IBD with a sensitivity of about 95%. In some embodiments, the PRI above the cutoff predicts an increase of IBD enriched cell types in an IBD-affected tissue as compared to a reference level in a tissue not affected by IBD with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase of IBD enriched cell types in an IBD-affected tissue as compared to a reference level in a tissue not affected by IBD with a specificity of at least 40%. The other embodiments described in this paragraph are provided for the various methods described herein including in Sections 2, 5.2 (e.g. the preceding paragraphs), and 7. In some embodiments, the PRI above the cutoff predicts an increase of IBD enriched cell types in an IBD-affected tissue as compared to a reference level in a tissue not affected by IBD with a specificity of at least 45%. In some embodiments, the PRI above the cutoff predicts an increase of IBD enriched cell types in an IBD-affected tissue as compared to a reference level in a tissue not affected by IBD with a specificity of at least 50%. In some embodiments, the PRI above the cutoff predicts an increase of IBD enriched cell types in an IBD-affected tissue as compared to a reference level in a tissue not affected by IBD with a specificity of at least 55%. In some embodiments, the PRI above the cutoff predicts an increase of IBD enriched cell types in an IBD-affected tissue as compared to a reference level in a tissue not affected by IBD with a specificity of at least 60%. In some embodiments, the PRI above the cutoff predicts an increase of IBD enriched cell types in an IBD-affected tissue as compared to a reference level in a tissue not affected by IBD with a specificity of at least 65%. In some embodiments, the PRI above the cutoff predicts an increase of IBD enriched cell types in an IBD-affected tissue as compared to a reference level in a tissue not affected by IBD with a specificity of at least 70%. In some embodiments, the PRI above the cutoff predicts an increase of IBD enriched cell types in an IBD-affected tissue as compared to a reference level in a tissue not affected by IBD with a specificity of at least 75%. In some embodiments, the PRI above the cutoff predicts an increase of IBD enriched cell types in an IBD-affected tissue as compared to a reference level in a tissue not affected by IBD with a specificity of at least 80%. In some embodiments, the PRI above the cutoff predicts an increase of IBD enriched cell types in an IBD-affected tissue as compared to a reference level in a tissue not affected by IBD with a specificity of at least 85%. In some embodiments, the PRI above the cutoff predicts an increase of IBD enriched cell types in an TED-affected tissue as compared to a reference level in a tissue not affected by IBD with a specificity of at least 90%. In some embodiments, the PRI above the cutoff predicts an increase of IBD enriched cell types in an IBD-affected tissue as compared to a reference level in a tissue not affected by IBD with a specificity of at least 95%. In some embodiments, the PRI above the cutoff predicts an increase of IBD enriched cell types in an IBD-affected tissue as compared to a reference level in a tissue not affected by IBD with a specificity of about 40%. In some embodiments, the PRI above the cutoff predicts an increase of IBD enriched cell types in an IBD-affected tissue as compared to a reference level in a tissue not affected by IBD with a specificity of about 45%. In some embodiments, the PRI above the cutoff predicts an increase of IBD enriched cell types in an IED-affected tissue as compared to a reference level in a tissue not affected by IBD with a specificity of about 50%. In some embodiments, the PRI above the cutoff predicts an increase of IBD enriched cell types in an IBD-affected tissue as compared to a reference level in a tissue not affected by IBD with a specificity of about 55%. In some embodiments, the PRI above the cutoff predicts an increase of IBD enriched cell types in an IBD-affected tissue as compared to a reference level in a tissue not affected by IBD with a specificity of about 60%. In some embodiments, the PRI above the cutoff predicts an increase of IBD enriched cell types in an TBD-affected tissue as compared to a reference level in a tissue not affected by IBD with a specificity of about 65%. In some embodiments, the PRI above the cutoff predicts an increase of IBD enriched cell types in an IBD-affected tissue as compared to a reference level in a tissue not affected by IBD with a specificity of about 70%. In some embodiments, the PRI above the cutoff predicts an increase of IBD enriched cell types in an IBD-affected tissue as compared to a reference level in a tissue not affected by IBD with a specificity of about 75%. In some embodiments, the PRI above the cutoff predicts an increase of IBD enriched cell types in an IBD-affected tissue as compared to a reference level in a tissue not affected by IBD with a specificity of about 80%. In some embodiments, the PRI above the cutoff predicts an increase of IBD enriched cell types in an IBD-affected tissue as compared to a reference level in a tissue not affected by IBD with a specificity of about 85%. In some embodiments, the PRI above the cutoff predicts an increase of IBD enriched cell types in an IBD-affected tissue as compared to a reference level in a tissue not affected by IBD with a specificity of about 90%. In some embodiments, the PRI above the cutoff predicts an increase of IBD enriched cell types in an IBD-affected tissue as compared to a reference level in a tissue not affected by IBD with a specificity of about 95%. In some embodiments, the PRI above the cutoff predicts an increase of IBD enriched cell types in an IBD-affected tissue as compared to a reference level in a tissue not affected by IBD with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase of IBD enriched cell types in an IBD-affected tissue as compared to a reference level in a tissue not affected by IBD with an accuracy of at least 50%. The other embodiments described in this paragraph are provided for the various methods described herein including in Sections 2, 5.2 (e.g. the preceding paragraphs), and 7. In some embodiments, the PRI above the cutoff predicts an increase of IBD enriched cell types in an IBD-affected tissue as compared to a reference level in a tissue not affected by IBD with an accuracy of at least 55%. In some embodiments, the PRI above the cutoff predicts an increase of IBD enriched cell types in an IBD-affected tissue as compared to a reference level in a tissue not affected by IBD with an accuracy of at least 60%. In some embodiments, the PRI above the cutoff predicts an increase of IBD enriched cell types in an IBD-affected tissue as compared to a reference level in a tissue not affected by IBD with an accuracy of at least 65%. In some embodiments, the PRI above the cutoff predicts an increase of IBD enriched cell types in an IBD-affected tissue as compared to a reference level in a tissue not affected by IBD with an accuracy of at least 70%. In some embodiments, the PRI above the cutoff predicts an increase of IBD enriched cell types in an IBD-affected tissue as compared to a reference level in a tissue not affected by IBD with an accuracy of at least 75%. In some embodiments, the PRI above the cutoff predicts an increase of IBD enriched cell types in an IBD-affected tissue as compared to a reference level in a tissue not affected by IBD with an accuracy of at least 80%. In some embodiments, the PRI above the cutoff predicts an increase of IBD enriched cell types in an IBD-affected tissue as compared to a reference level in a tissue not affected by IBD with an accuracy of at least 85%. In some embodiments, the PRI above the cutoff predicts an increase of IBD enriched cell types in an TED-affected tissue as compared to a reference level in a tissue not affected by IBD with an accuracy of at least 90%. In some embodiments, the PRI above the cutoff predicts an increase of IBD enriched cell types in an IBD-affected tissue as compared to a reference level in a tissue not affected by IBD with an accuracy of at least 95%. In some embodiments, the PRI above the cutoff predicts an increase of IBD enriched cell types in an IBD-affected tissue as compared to a reference level in a tissue not affected by IBD with an accuracy of about 50%. In some embodiments, the PRI above the cutoff predicts an increase of IBD enriched cell types in an 113D-affected tissue as compared to a reference level in a tissue not affected by IBD with an accuracy of about 55%. In some embodiments, the PRI above the cutoff predicts an increase of IBD enriched cell types in an IBD-affected tissue as compared to a reference level in a tissue not affected by IBD with an accuracy of about 60%. In some embodiments, the PRI above the cutoff predicts an increase of IBD enriched cell types in an IBD-affected tissue as compared to a reference level in a tissue not affected by IBD with an accuracy of about 65%. In some embodiments, the PRI above the cutoff predicts an increase of IBD enriched cell types in an IBD-affected tissue as compared to a reference level in a tissue not affected by IBD with an accuracy of about 70%. In some embodiments, the PRI above the cutoff predicts an increase of IBD enriched cell types in an IBD-affected tissue as compared to a reference level in a tissue not affected by IBD with an accuracy of about 75%. In some embodiments, the PRI above the cutoff predicts an increase of IBD enriched cell types in an IBD-affected tissue as compared to a reference level in a tissue not affected by IBD with an accuracy of about 80%. In some embodiments, the PRI above the cutoff predicts an increase of IBD enriched cell types in an IBD-affected tissue as compared to a reference level in a tissue not affected by IBD with an accuracy of about 85%. In some embodiments, the PRI above the cutoff predicts an increase of IBD enriched cell types in an IBD-affected tissue as compared to a reference level in a tissue not affected by IBD with an accuracy of about 90%. In some embodiments, the PRI above the cutoff predicts an increase of IBD enriched cell types in an IBD-affected tissue as compared to a reference level in a tissue not affected by IBD with an accuracy of about 95%. In some embodiments, the PRI above the cutoff predicts an increase of IBD enriched cell types in an IBD-affected tissue as compared to a reference level in a tissue not affected by IBD with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a decrease of one or more IBD depleted cell types in an IBD-affected tissue as compared to a reference level in a tissue not affected by IBD with a positive predictive value of at least 25%. The other embodiments described in this paragraph are provided for the various methods described herein including in Sections 2, 5.2 (e.g. the preceding paragraphs), and 7. In some embodiments, the PRI above the cutoff predicts a decrease of one or more IBD depleted cell types in an IBD-affected tissue as compared to a reference level in a tissue not affected by IBD with a positive predictive value of at least 26%. In some embodiments, the PRI above the cutoff predicts a decrease of one or more IBD depleted cell types in an IBD-affected tissue as compared to a reference level in a tissue not affected by IBD with a positive predictive value of at least 27%. In some embodiments, the PRI above the cutoff predicts a decrease of one or more IBD depleted cell types in an IBD-affected tissue as compared to a reference level in a tissue not affected by IBD with a positive predictive value of at least 28%. In some embodiments, the PRI above the cutoff predicts a decrease of one or more IBD depleted cell types in an IBD-affected tissue as compared to a reference level in a tissue not affected by IBD with a positive predictive value of at least 29%. In some embodiments, the PRI above the cutoff predicts a decrease of one or more IBD depleted cell types in an IBD-affected tissue as compared to a reference level in a tissue not affected by IBD with a positive predictive value of at least 30%. In some embodiments, the PRI above the cutoff predicts a decrease of one or more IBD depleted cell types in an IBD-affected tissue as compared to a reference level in a tissue not affected by IBD with a positive predictive value of at least 31%. In some embodiments, the PRI above the cutoff predicts a decrease of one or more IBD depleted cell types in an IBD-affected tissue as compared to a reference level in a tissue not affected by IBD with a positive predictive value of at least 32%. In some embodiments, the PRI above the cutoff predicts a decrease of one or more IBD depleted cell types in an IBD-affected tissue as compared to a reference level in a tissue not affected by IBD with a positive predictive value of at least 33%. In some embodiments, the PRI above the cutoff predicts a decrease of one or more IBD depleted cell types in an IBD-affected tissue as compared to a reference level in a tissue not affected by IBD with a positive predictive value of at least 34%. In some embodiments, the PRI above the cutoff predicts a decrease of one or more IBD depleted cell types in an IBD-affected tissue as compared to a reference level in a tissue not affected by IBD with a positive predictive value of at least 35%. In some embodiments, the PRI above the cutoff predicts a decrease of one or more IBD depleted cell types in an IBD-affected tissue as compared to a reference level in a tissue not affected by IBD with a positive predictive value of at least 40%. In some embodiments, the PRI above the cutoff predicts a decrease of one or more IBD depleted cell types in an IBD-affected tissue as compared to a reference level in a tissue not affected by IBD with a positive predictive value of at least 45%. In some embodiments, the PRI above the cutoff predicts a decrease of one or more IBD depleted cell types in an IBD-affected tissue as compared to a reference level in a tissue not affected by IBD with a positive predictive value of at least 50%. In some embodiments, the PRI above the cutoff predicts a decrease of one or more IBD depleted cell types in an IBD-affected tissue as compared to a reference level in a tissue not affected by IBD with a positive predictive value of at least 51%. In some embodiments, the PRI above the cutoff predicts a decrease of one or more IBD depleted cell types in an IBD-affected tissue as compared to a reference level in a tissue not affected by IBD with a positive predictive value of at least 55%. In some embodiments, the PRI above the cutoff predicts a decrease of one or more IBD depleted cell types in an IBD-affected tissue as compared to a reference level in a tissue not affected by IBD with a positive predictive value of at least 60%. In some embodiments, the PRI above the cutoff predicts a decrease of one or more IBD depleted cell types in an IBD-affected tissue as compared to a reference level in a tissue not affected by IBD with a positive predictive value of at least 65%. In some embodiments, the PRI above the cutoff predicts a decrease of one or more IBD depleted cell types in an IBD-affected tissue as compared to a reference level in a tissue not affected by IBD with a positive predictive value of at least 70%. In some embodiments, the PRI above the cutoff predicts a decrease of one or more IBD depleted cell types in an IBD-affected tissue as compared to a reference level in a tissue not affected by IBD with a positive predictive value of at least 75%. In some embodiments, the PRI above the cutoff predicts a decrease of one or more IBD depleted cell types in an IBD-affected tissue as compared to a reference level in a tissue not affected by IBD with a positive predictive value of at least 80%. In some embodiments, the PRI above the cutoff predicts a decrease of one or more IBD depleted cell types in an IBD-affected tissue as compared to a reference level in a tissue not affected by IBD with a positive predictive value of at least 85%. In some embodiments, the PRI above the cutoff predicts a decrease of one or more IBD depleted cell types in an IBD-affected tissue as compared to a reference level in a tissue not affected by IBD with a positive predictive value of at least 90%. In some embodiments, the PRI above the cutoff predicts a decrease of one or more IBD depleted cell types in an IBD-affected tissue as compared to a reference level in a tissue not affected by IBD with a positive predictive value of at least 95%. In some embodiments, the PRI above the cutoff predicts a decrease of one or more IBD depleted cell types in an IBD-affected tissue as compared to a reference level in a tissue not affected by IBD with a positive predictive value of about 25%. In some embodiments, the PRI above the cutoff predicts a decrease of one or more IBD depleted cell types in an IBD-affected tissue as compared to a reference level in a tissue not affected by IBD with a positive predictive value of about 26%. In some embodiments, the PRI above the cutoff predicts a decrease of one or more IBD depleted cell types in an IBD-affected tissue as compared to a reference level in a tissue not affected by IBD with a positive predictive value of about 27%. In some embodiments, the PRI above the cutoff predicts a decrease of one or more IBD depleted cell types in an IBD-affected tissue as compared to a reference level in a tissue not affected by IBD with a positive predictive value of about 28%. In some embodiments, the PRI above the cutoff predicts a decrease of one or more IBD depleted cell types in an IBD-affected tissue as compared to a reference level in a tissue not affected by IBD with a positive predictive value of about 29%. In some embodiments, the PRI above the cutoff predicts a decrease of one or more IBD depleted cell types in an IBD-affected tissue as compared to a reference level in a tissue not affected by IBD with a positive predictive value of about 30%. In some embodiments, the PRI above the cutoff predicts a decrease of one or more IBD depleted cell types in an IBD-affected tissue as compared to a reference level in a tissue not affected by IBD with a positive predictive value of about 31%. In some embodiments, the PRI above the cutoff predicts a decrease of one or more IBD depleted cell types in an IBD-affected tissue as compared to a reference level in a tissue not affected by IBD with a positive predictive value of about 32%. In some embodiments, the PRI above the cutoff predicts a decrease of one or more IBD depleted cell types in an IBD-affected tissue as compared to a reference level in a tissue not affected by IBD with a positive predictive value of about 33%. In some embodiments, the PRI above the cutoff predicts a decrease of one or more IBD depleted cell types in an IBD-affected tissue as compared to a reference level in a tissue not affected by IBD with a positive predictive value of about 34%. In some embodiments, the PRI above the cutoff predicts a decrease of one or more IBD depleted cell types in an IBD-affected tissue as compared to a reference level in a tissue not affected by IBD with a positive predictive value of about 35%. In some embodiments, the PRI above the cutoff predicts a decrease of one or more IBD depleted cell types in an IBD-affected tissue as compared to a reference level in a tissue not affected by IBD with a positive predictive value of about 40%. In some embodiments, the PRI above the cutoff predicts a decrease of one or more IBD depleted cell types in an IBD-affected tissue as compared to a reference level in a tissue not affected by IBD with a positive predictive value of about 45%. In some embodiments, the PRI above the cutoff predicts a decrease of one or more IBD depleted cell types in an IBD-affected tissue as compared to a reference level in a tissue not affected by IBD with a positive predictive value of about 50%. In some embodiments, the PRI above the cutoff predicts a decrease of one or more IBD depleted cell types in an IBD-affected tissue as compared to a reference level in a tissue not affected by IBD with a positive predictive value of about 51%. In some embodiments, the PRI above the cutoff predicts a decrease of one or more IBD depleted cell types in an IBD-affected tissue as compared to a reference level in a tissue not affected by IBD with a positive predictive value of about 55%. In some embodiments, the PRI above the cutoff predicts a decrease of one or more IBD depleted cell types in an IBD-affected tissue as compared to a reference level in a tissue not affected by IBD with a positive predictive value of about 60%. In some embodiments, the PRI above the cutoff predicts a decrease of one or more IBD depleted cell types in an IBD-affected tissue as compared to a reference level in a tissue not affected by IBD with a positive predictive value of about 65%. In some embodiments, the PRI above the cutoff predicts a decrease of one or more TED depleted cell types in an IBD-affected tissue as compared to a reference level in a tissue not affected by IBD with a positive predictive value of about 70%. In some embodiments, the PRI above the cutoff predicts a decrease of one or more IBD depleted cell types in an TED-affected tissue as compared to a reference level in a tissue not affected by IBD with a positive predictive value of about 75%. In some embodiments, the PRI above the cutoff predicts a decrease of one or more IBD depleted cell types in an IBD-affected tissue as compared to a reference level in a tissue not affected by IBD with a positive predictive value of about 80%. In some embodiments, the PRI above the cutoff predicts a decrease of one or more IBD depleted cell types in an IBD-affected tissue as compared to a reference level in a tissue not affected by IBD with a positive predictive value of about 85%. In some embodiments, the PRI above the cutoff predicts a decrease of one or more IBD depleted cell types in an IBD-affected tissue as compared to a reference level in a tissue not affected by IBD with a positive predictive value of about 90%. In some embodiments, the PRI above the cutoff predicts a decrease of one or more IBD depleted cell types in an IBD-affected tissue as compared to a reference level in a tissue not affected by IBD with a positive predictive value of about 95%. In some embodiments, the PRI above the cutoff predicts a decrease of one or more IBD depleted cell types in an IBD-affected tissue as compared to a reference level in a tissue not affected by IBD with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a decrease of one or more IBD depleted cell types in an IBD-affected tissue as compared to a reference level in a tissue not affected by IBD with a negative predictive value of at least 40%. The other embodiments described in this paragraph are provided for the various methods described herein including in Sections 2, 5.2 (e.g. the preceding paragraphs), and 7. In some embodiments, the PRI above the cutoff predicts a decrease of one or more IBD depleted cell types in an IBD-affected tissue as compared to a reference level in a tissue not affected by IBD with a negative predictive value of at least 45%. In some embodiments, the PRI above the cutoff predicts a decrease of one or more IBD depleted cell types in an IBD-affected tissue as compared to a reference level in a tissue not affected by IBD with a negative predictive value of at least 50%. In some embodiments, the PRI above the cutoff predicts a decrease of one or more IBD depleted cell types in an TBD-affected tissue as compared to a reference level in a tissue not affected by IBD with a negative predictive value of at least 55%. In some embodiments, the PRI above the cutoff predicts a decrease of one or more IBD depleted cell types in an IBD-affected tissue as compared to a reference level in a tissue not affected by IBD with a negative predictive value of at least 60%. In some embodiments, the PRI above the cutoff predicts a decrease of one or more IBD depleted cell types in an IBD-affected tissue as compared to a reference level in a tissue not affected by IBD with a negative predictive value of at least 65%. In some embodiments, the PRI above the cutoff predicts a decrease of one or more IBD depleted cell types in an IBD-affected tissue as compared to a reference level in a tissue not affected by IBD with a negative predictive value of at least 70%. In some embodiments, the PRI above the cutoff predicts a decrease of one or more IBD depleted cell types in an IBD-affected tissue as compared to a reference level in a tissue not affected by IBD with a negative predictive value of at least 75%. In some embodiments, the PRI above the cutoff predicts a decrease of one or more IBD depleted cell types in an IBD-affected tissue as compared to a reference level in a tissue not affected by IBD with a negative predictive value of at least 80%. In some embodiments, the PRI above the cutoff predicts a decrease of one or more IBD depleted cell types in an IBD-affected tissue as compared to a reference level in a tissue not affected by IBD with a negative predictive value of at least 85%. In some embodiments, the PRI above the cutoff predicts a decrease of one or more IBD depleted cell types in an IBD-affected tissue as compared to a reference level in a tissue not affected by IBD with a negative predictive value of at least 90%. In some embodiments, the PRI above the cutoff predicts a decrease of one or more IBD depleted cell types in an IBD-affected tissue as compared to a reference level in a tissue not affected by IBD with a negative predictive value of at least 95%. In some embodiments, the PRI above the cutoff predicts a decrease of one or more IBD depleted cell types in an IBD-affected tissue as compared to a reference level in a tissue not affected by IBD with a negative predictive value of about 40%. In some embodiments, the PRI above the cutoff predicts a decrease of one or more IBD depleted cell types in an IBD-affected tissue as compared to a reference level in a tissue not affected by IBD with a negative predictive value of about 45%. In some embodiments, the PRI above the cutoff predicts a decrease of one or more IBD depleted cell types in an IBD-affected tissue as compared to a reference level in a tissue not affected by IBD with a negative predictive value of about 50%. In some embodiments, the PRI above the cutoff predicts a decrease of one or more IBD depleted cell types in an IBD-affected tissue as compared to a reference level in a tissue not affected by IBD with a negative predictive value of about 55%. In some embodiments, the PRI above the cutoff predicts a decrease of one or more IBD depleted cell types in an IBD-affected tissue as compared to a reference level in a tissue not affected by IBD with a negative predictive value of about 60%. In some embodiments, the PRI above the cutoff predicts a decrease of one or more IBD depleted cell types in an IBD-affected tissue as compared to a reference level in a tissue not affected by IBD with a negative predictive value of about 65%. In some embodiments, the PRI above the cutoff predicts a decrease of one or more IBD depleted cell types in an IBD-affected tissue as compared to a reference level in a tissue not affected by IBD with a negative predictive value of about 70%. In some embodiments, the PRI above the cutoff predicts a decrease of one or more IBD depleted cell types in an IBD-affected tissue as compared to a reference level in a tissue not affected by IBD with a negative predictive value of about 75%. In some embodiments, the PRI above the cutoff predicts a decrease of one or more IBD depleted cell types in an IBD-affected tissue as compared to a reference level in a tissue not affected by IBD with a negative predictive value of about 80%. In some embodiments, the PRI above the cutoff predicts a decrease of one or more IBD depleted cell types in an IBD-affected tissue as compared to a reference level in a tissue not affected by IBD with a negative predictive value of about 85%. In some embodiments, the PRI above the cutoff predicts a decrease of one or more IBD depleted cell types in an TBD-affected tissue as compared to a reference level in a tissue not affected by IBD with a negative predictive value of about 90%. In some embodiments, the PRI above the cutoff predicts a decrease of one or more IBD depleted cell types in an IBD-affected tissue as compared to a reference level in a tissue not affected by IBD with a negative predictive value of about 95%. In some embodiments, the PRI above the cutoff predicts a decrease of one or more IBD depleted cell types in an IBD-affected tissue as compared to a reference level in a tissue not affected by IBD with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a decrease of one or more IBD depleted cell types in an IBD-affected tissue as compared to a reference level in a tissue not affected by IBD with a sensitivity of at least 20%. The other embodiments described in this paragraph are provided for the various methods described herein including in Sections 2, 5.2 (e.g. the preceding paragraphs), and 7. In some embodiments, the PRI above the cutoff predicts a decrease of one or more IBD depleted cell types in an IBD-affected tissue as compared to a reference level in a tissue not affected by IBD with a sensitivity of at least 25%. In some embodiments, the PRI above the cutoff predicts a decrease of one or more IBD depleted cell types in an IBD-affected tissue as compared to a reference level in a tissue not affected by IBD with a sensitivity of at least 30%. In some embodiments, the PRI above the cutoff predicts a decrease of one or more IBD depleted cell types in an IBD-affected tissue as compared to a reference level in a tissue not affected by IBD with a sensitivity of at least 35%. In some embodiments, the PRI above the cutoff predicts a decrease of one or more IBD depleted cell types in an IBD-affected tissue as compared to a reference level in a tissue not affected by IBD with a sensitivity of at least 40%. In some embodiments, the PRI above the cutoff predicts a decrease of one or more IBD depleted cell types in an IBD-affected tissue as compared to a reference level in a tissue not affected by IBD with a sensitivity of at least 45%. In some embodiments, the PRI above the cutoff predicts a decrease of one or more IBD depleted cell types in an IBD-affected tissue as compared to a reference level in a tissue not affected by IBD with a sensitivity of at least 50%. In some embodiments, the PRI above the cutoff predicts a decrease of one or more IBD depleted cell types in an IBD-affected tissue as compared to a reference level in a tissue not affected by IBD with a sensitivity of at least 55%. In some embodiments, the PRI above the cutoff predicts a decrease of one or more IBD depleted cell types in an IBD-affected tissue as compared to a reference level in a tissue not affected by IBD with a sensitivity of at least 60%. In some embodiments, the PRI above the cutoff predicts a decrease of one or more IBD depleted cell types in an IBD-affected tissue as compared to a reference level in a tissue not affected by IBD with a sensitivity of at least 65%. In some embodiments, the PRI above the cutoff predicts a decrease of one or more IBD depleted cell types in an IBD-affected tissue as compared to a reference level in a tissue not affected by IBD with a sensitivity of at least 70%. In some embodiments, the PRI above the cutoff predicts a decrease of one or more IBD depleted cell types in an IBD-affected tissue as compared to a reference level in a tissue not affected by IBD with a sensitivity of at least 75%. In some embodiments, the PRI above the cutoff predicts a decrease of one or more IBD depleted cell types in an IBD-affected tissue as compared to a reference level in a tissue not affected by IBD with a sensitivity of at least 80%. In some embodiments, the PRI above the cutoff predicts a decrease of one or more IBD depleted cell types in an IBD-affected tissue as compared to a reference level in a tissue not affected by IBD with a sensitivity of at least 85%. In some embodiments, the PRI above the cutoff predicts a decrease of one or more IBD depleted cell types in an IBD-affected tissue as compared to a reference level in a tissue not affected by IBD with a sensitivity of at least 90%. In some embodiments, the PRI above the cutoff predicts a decrease of one or more IBD depleted cell types in an IBD-affected tissue as compared to a reference level in a tissue not affected by IBD with a sensitivity of at least 95%. In some embodiments, the PRI above the cutoff predicts a decrease of one or more IBD depleted cell types in an IBD-affected tissue as compared to a reference level in a tissue not affected by IBD with a sensitivity of about 20%. In some embodiments, the PRI above the cutoff predicts a decrease of one or more IBD depleted cell types in an IBD-affected tissue as compared to a reference level in a tissue not affected by IBD with a sensitivity of about 25%. In some embodiments, the PRI above the cutoff predicts a decrease of one or more IBD depleted cell types in an IBD-affected tissue as compared to a reference level in a tissue not affected by IBD with a sensitivity of about 30%. In some embodiments, the PRI above the cutoff predicts a decrease of one or more IBD depleted cell types in an IBD-affected tissue as compared to a reference level in a tissue not affected by IBD with a sensitivity of about 35%. In some embodiments, the PRI above the cutoff predicts a decrease of one or more IBD depleted cell types in an IBD-affected tissue as compared to a reference level in a tissue not affected by IBD with a sensitivity of about 40%. In some embodiments, the PRI above the cutoff predicts a decrease of one or more IBD depleted cell types in an IBD-affected tissue as compared to a reference level in a tissue not affected by IBD with a sensitivity of about 45%. In some embodiments, the PRI above the cutoff predicts a decrease of one or more IBD depleted cell types in an IBD-affected tissue as compared to a reference level in a tissue not affected by IBD with a sensitivity of about 50%. In some embodiments, the PRI above the cutoff predicts a decrease of one or more IBD depleted cell types in an IBD-affected tissue as compared to a reference level in a tissue not affected by IBD with a sensitivity of about 55%. In some embodiments, the PRI above the cutoff predicts a decrease of one or more IBD depleted cell types in an IBD-affected tissue as compared to a reference level in a tissue not affected by IBD with a sensitivity of about 60%. In some embodiments, the PRI above the cutoff predicts a decrease of one or more IBD depleted cell types in an IBD-affected tissue as compared to a reference level in a tissue not affected by IBD with a sensitivity of about 65%. In some embodiments, the PRI above the cutoff predicts a decrease of one or more IBD depleted cell types in an IBD-affected tissue as compared to a reference level in a tissue not affected by IBD with a sensitivity of about 70%. In some embodiments, the PRI above the cutoff predicts a decrease of one or more IBD depleted cell types in an IBD-affected tissue as compared to a reference level in a tissue not affected by IBD with a sensitivity of about 75%. In some embodiments, the PRI above the cutoff predicts a decrease of one or more IBD depleted cell types in an IBD-affected tissue as compared to a reference level in a tissue not affected by IBD with a sensitivity of about 80%. In some embodiments, the PRI above the cutoff predicts a decrease of one or more IBD depleted cell types in an IBD-affected tissue as compared to a reference level in a tissue not affected by IBD with a sensitivity of about 85%. In some embodiments, the PRI above the cutoff predicts a decrease of one or more IBD depleted cell types in an IBD-affected tissue as compared to a reference level in a tissue not affected by IBD with a sensitivity of about 90%. In some embodiments, the PRI above the cutoff predicts a decrease of one or more IBD depleted cell types in an IBD-affected tissue as compared to a reference level in a tissue not affected by IBD with a sensitivity of about 95%. In some embodiments, the PRI above the cutoff predicts a decrease of one or more IBD depleted cell types in an IBD-affected tissue as compared to a reference level in a tissue not affected by IBD with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a decrease of one or more IBD depleted cell types in an IBD-affected tissue as compared to a reference level in a tissue not affected by IBD with a specificity of at least 40%. The other embodiments described in this paragraph are provided for the various methods described herein including in Sections 2, 5.2 (e.g. the preceding paragraphs), and 7. In some embodiments, the PRI above the cutoff predicts a decrease of one or more IBD depleted cell types in an IBD-affected tissue as compared to a reference level in a tissue not affected by IBD with a specificity of at least 45%. In some embodiments, the PRI above the cutoff predicts a decrease of one or more IBD depleted cell types in an IBD-affected tissue as compared to a reference level in a tissue not affected by IBD with a specificity of at least 50%. In some embodiments, the PRI above the cutoff predicts a decrease of one or more IBD depleted cell types in an IBD-affected tissue as compared to a reference level in a tissue not affected by IBD with a specificity of at least 55%. In some embodiments, the PRI above the cutoff predicts a decrease of one or more IBD depleted cell types in an IBD-affected tissue as compared to a reference level in a tissue not affected by IBD with a specificity of at least 60%. In some embodiments, the PRI above the cutoff predicts a decrease of one or more IBD depleted cell types in an IBD-affected tissue as compared to a reference level in a tissue not affected by IBD with a specificity of at least 65%. In some embodiments, the PRI above the cutoff predicts a decrease of one or more IBD depleted cell types in an IBD-affected tissue as compared to a reference level in a tissue not affected by IBD with a specificity of at least 70%. In some embodiments, the PRI above the cutoff predicts a decrease of one or more IBD depleted cell types in an IBD-affected tissue as compared to a reference level in a tissue not affected by IBD with a specificity of at least 75%. In some embodiments, the PRI above the cutoff predicts a decrease of one or more IBD depleted cell types in an IBD-affected tissue as compared to a reference level in a tissue not affected by IBD with a specificity of at least 80%. In some embodiments, the PRI above the cutoff predicts a decrease of one or more IBD depleted cell types in an IBD-affected tissue as compared to a reference level in a tissue not affected by IBD with a specificity of at least 85%. In some embodiments, the PRI above the cutoff predicts a decrease of one or more IBD depleted cell types in an IBD-affected tissue as compared to a reference level in a tissue not affected by IBD with a specificity of at least 90%. In some embodiments, the PRI above the cutoff predicts a decrease of one or more IBD depleted cell types in an IBD-affected tissue as compared to a reference level in a tissue not affected by IBD with a specificity of at least 95%. In some embodiments, the PRI above the cutoff predicts a decrease of one or more IBD depleted cell types in an IBD-affected tissue as compared to a reference level in a tissue not affected by IBD with a specificity of about 40%. In some embodiments, the PRI above the cutoff predicts a decrease of one or more IBD depleted cell types in an IBD-affected tissue as compared to a reference level in a tissue not affected by IBD with a specificity of about 45%. In some embodiments, the PRI above the cutoff predicts a decrease of one or more IBD depleted cell types in an IBD-affected tissue as compared to a reference level in a tissue not affected by IBD with a specificity of about 50%. In some embodiments, the PRI above the cutoff predicts a decrease of one or more IBD depleted cell types in an IBD-affected tissue as compared to a reference level in a tissue not affected by IBD with a specificity of about 55%. In some embodiments, the PRI above the cutoff predicts a decrease of one or more IBD depleted cell types in an IBD-affected tissue as compared to a reference level in a tissue not affected by IBD with a specificity of about 60%. In some embodiments, the PRI above the cutoff predicts a decrease of one or more IBD depleted cell types in an IBD-affected tissue as compared to a reference level in a tissue not affected by IBD with a specificity of about 65%. In some embodiments, the PRI above the cutoff predicts a decrease of one or more IBD depleted cell types in an IBD-affected tissue as compared to a reference level in a tissue not affected by IBD with a specificity of about 70%. In some embodiments, the PRI above the cutoff predicts a decrease of one or more IBD depleted cell types in an IBD-affected tissue as compared to a reference level in a tissue not affected by IBD with a specificity of about 75%. In some embodiments, the PRI above the cutoff predicts a decrease of one or more IBD depleted cell types in an IBD-affected tissue as compared to a reference level in a tissue not affected by IBD with a specificity of about 80%. In some embodiments, the PRI above the cutoff predicts a decrease of one or more IBD depleted cell types in an IBD-affected tissue as compared to a reference level in a tissue not affected by IBD with a specificity of about 85%. In some embodiments, the PRI above the cutoff predicts a decrease of one or more IBD depleted cell types in an IBD-affected tissue as compared to a reference level in a tissue not affected by IBD with a specificity of about 90%. In some embodiments, the PRI above the cutoff predicts a decrease of one or more IBD depleted cell types in an IBD-affected tissue as compared to a reference level in a tissue not affected by IBD with a specificity of about 95%. In some embodiments, the PRI above the cutoff predicts a decrease of one or more IBD depleted cell types in an IBD-affected tissue as compared to a reference level in a tissue not affected by IBD with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts a decrease of one or more IBD depleted cell types in an IBD-affected tissue as compared to a reference level in a tissue not affected by IBD with an accuracy of at least 50%. The other embodiments described in this paragraph are provided for the various methods described herein including in Sections 2, 5.2 (e.g. the preceding paragraphs), and 7. In some embodiments, the PRI above the cutoff predicts a decrease of one or more IBD depleted cell types in an IBD-affected tissue as compared to a reference level in a tissue not affected by IBD with an accuracy of at least 55%. In some embodiments, the PRI above the cutoff predicts a decrease of one or more IBD depleted cell types in an IBD-affected tissue as compared to a reference level in a tissue not affected by IBD with an accuracy of at least 60%. In some embodiments, the PRI above the cutoff predicts a decrease of one or more IBD depleted cell types in an IBD-affected tissue as compared to a reference level in a tissue not affected by IBD with an accuracy of at least 65%. In some embodiments, the PRI above the cutoff predicts a decrease of one or more IBD depleted cell types in an IBD-affected tissue as compared to a reference level in a tissue not affected by IBD with an accuracy of at least 70%. In some embodiments, the PRI above the cutoff predicts a decrease of one or more IBD depleted cell types in an IBD-affected tissue as compared to a reference level in a tissue not affected by IBD with an accuracy of at least 75%. In some embodiments, the PRI above the cutoff predicts a decrease of one or more IBD depleted cell types in an IBD-affected tissue as compared to a reference level in a tissue not affected by IBD with an accuracy of at least 80%. In some embodiments, the PRI above the cutoff predicts a decrease of one or more IBD depleted cell types in an IBD-affected tissue as compared to a reference level in a tissue not affected by IBD with an accuracy of at least 85%. In some embodiments, the PRI above the cutoff predicts a decrease of one or more IBD depleted cell types in an IBD-affected tissue as compared to a reference level in a tissue not affected by IBD with an accuracy of at least 90%. In some embodiments, the PRI above the cutoff predicts a decrease of one or more IBD depleted cell types in an IBD-affected tissue as compared to a reference level in a tissue not affected by IBD with an accuracy of at least 95%. In some embodiments, the PRI above the cutoff predicts a decrease of one or more IBD depleted cell types in an IBD-affected tissue as compared to a reference level in a tissue not affected by IBD with an accuracy of about 50%. In some embodiments, the PRI above the cutoff predicts a decrease of one or more IBD depleted cell types in an IBD-affected tissue as compared to a reference level in a tissue not affected by IBD with an accuracy of about 55%. In some embodiments, the PRI above the cutoff predicts a decrease of one or more IBD depleted cell types in an IBD-affected tissue as compared to a reference level in a tissue not affected by IBD with an accuracy of about 60%. In some embodiments, the PRI above the cutoff predicts a decrease of one or more IBD depleted cell types in an IBD-affected tissue as compared to a reference level in a tissue not affected by IBD with an accuracy of about 65%. In some embodiments, the PRI above the cutoff predicts a decrease of one or more IBD depleted cell types in an IBD-affected tissue as compared to a reference level in a tissue not affected by IBD with an accuracy of about 70%. In some embodiments, the PRI above the cutoff predicts a decrease of one or more IBD depleted cell types in an IBD-affected tissue as compared to a reference level in a tissue not affected by IBD with an accuracy of about 75%. In some embodiments, the PRI above the cutoff predicts a decrease of one or more IBD depleted cell types in an IBD-affected tissue as compared to a reference level in a tissue not affected by IBD with an accuracy of about 80%. In some embodiments, the PRI above the cutoff predicts a decrease of one or more IBD depleted cell types in an IBD-affected tissue as compared to a reference level in a tissue not affected by IBD with an accuracy of about 85%. In some embodiments, the PRI above the cutoff predicts a decrease of one or more IBD depleted cell types in an IBD-affected tissue as compared to a reference level in a tissue not affected by IBD with an accuracy of about 90%. In some embodiments, the PRI above the cutoff predicts a decrease of one or more IBD depleted cell types in an IBD-affected tissue as compared to a reference level in a tissue not affected by IBD with an accuracy of about 95%. In some embodiments, the PRI above the cutoff predicts a decrease of one or more IBD depleted cell types in an IBD-affected tissue as compared to a reference level in a tissue not affected by IBD with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%. In some embodiments of the methods provided herein including in this paragraph, the PRI is the MRS. In certain embodiments of the methods provided herein including in this paragraph the PRI is the RPS. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase in TL1A protein expression in a sample obtained from a subject as compared to a reference level of TL1A protein expression (e.g., level in a tissue not affected by IBD) with a positive predictive value of at least 25%. The other embodiments described in this paragraph are provided for the various methods described herein including in Sections 2, 5.2 (e.g. the preceding paragraphs), and 7. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 26%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 27%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 28%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 29%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 30%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 31%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 32%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 33%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 34%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 35%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 40%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 45%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 50%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 51%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 55%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 60%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 65%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 70%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 75%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 80%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 85%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 90%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of at least 95%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 25%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 26%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 27%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 28%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 29%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 30%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 31%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 32%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 33%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 34%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 35%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 40%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 45%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 50%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 51%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 55%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 60%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 65%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 70%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 75%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 80%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 85%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 90%. In some embodiments, the PRI above the cutoff predicts an increase in TL1A protein expression in a sample obtained from a subject as compared to a reference level of TL1A protein expression (e.g., level in a tissue not affected by IBD) with a positive predictive value of about 95%. In some embodiments, the PRI above the cutoff predicts an increase in TL1A protein expression in a sample obtained from a subject as compared to a reference level of TL1A protein expression (e.g., level in a tissue not affected by IBD) with a positive predictive value from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 25% to 70%, from 30% to 70%, from 35% to 70%, from 40% to 70%, from 45% to 70%, from 50% to 70%, from 55% to 70%, from 60% to 70%, from 65% to 70%, from 25% to 65%, from 30% to 65%, from 35% to 65%, from 40% to 65%, from 45% to 65%, from 50% to 65%, from 55% to 65%, from 60% to 65%, from 25% to 60%, from 30% to 60%, from 35% to 60%, from 40% to 60%, from 45% to 60%, from 50% to 60%, from 55% to 60%, from 25% to 55%, from 30% to 55%, from 35% to 55%, from 40% to 55%, from 45% to 55%, from 50% to 55%, from 25% to 50%, from 30% to 50%, from 35% to 50%, from 40% to 50%, from 45% to 50%, from 25% to 45%, from 30% to 45%, from 35% to 45%, from 40% to 45%, from 25% to 40%, from 30% to 40%, from 35% to 40%, from 25% to 35%, from 30% to 35%, or from 25% to 30%. In some embodiments of the methods provided herein including in this paragraph, the PRI is the MRS. In certain embodiments of the methods provided herein including in this paragraph the PRI is the RPS. In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff, or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase in TL1A protein expression in a sample obtained from a subject as compared to a reference level of TL1A protein expression (e.g., level in a tissue not affected by IBD) with a negative predictive value of at least 40%. The other embodiments described in this paragraph are provided for the various methods described herein including in Sections 2, 5.2 (e.g. the preceding paragraphs), and 7. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 45%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 50%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 55%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 60%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 65%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 70%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 75%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 80%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 85%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 90%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of at least 95%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 45%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 50%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 50%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 55%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 60%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 65%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 70%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 75%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 80%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 85%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 90%. In some embodiments, the PRI above the cutoff predicts an increase in TL1A protein expression in a sample obtained from a subject as compared to a reference level of TL1A protein expression (e.g., level in a tissue not affected by IBD) with a negative predictive value of about 95%. In some embodiments, the PRI above the cutoff predicts an increase in TL1A protein expression in a sample obtained from a subject as compared to a reference level of TL1A protein expression (e.g., level in a tissue not affected by IBD) with a negative predictive value from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase in TL1A protein expression in a sample obtained from a subject as compared to a reference level of TL1A protein expression (e.g., level in a tissue not affected by IBD) with a sensitivity of at least 20%. The other embodiments described in this paragraph are provided for the various methods described herein including in Sections 2, 5.2 (e.g. the preceding paragraphs), and 7. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 25%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 30%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 35%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 40%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 45%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 50%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 55%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 60%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 65%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 70%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 75%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 80%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 85%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 90%. In some embodiments, the PRI above the cutoff predicts an increase in TL1A protein expression in a sample obtained from a subject as compared to a reference level of TL1A protein expression (e.g., level in a tissue not affected by IBD) with a sensitivity of at least 95%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 20%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 25%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 30%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 35%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 40%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 45%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 50%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 55%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 60%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 65%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 70%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 75%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 80%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 85%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 90%. In some embodiments, the PRI above the cutoff predicts an increase in TL1A protein expression in a sample obtained from a subject as compared to a reference level of TL1A protein expression (e.g., level in a tissue not affected by IBD) with a sensitivity of about 95%. In some embodiments, the PRI above the cutoff predicts an increase in TL1A protein expression in a sample obtained from a subject as compared to a reference level of TL1A protein expression (e.g., level in a tissue not affected by IBD) with a sensitivity from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 85% to 95%, from 90% to 95%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 85% to 90%, from 20% to 85%, from 25% to 85%, from 30% to 85%, from 35% to 85%, from 40% to 85%, from 45% to 85%, from 50% to 85%, from 55% to 85%, from 60% to 85%, from 65% to 85%, from 70% to 85%, from 75% to 85%, from 80% to 85%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 20% to 75%, from 25% to 75%, from 30% to 75%, from 35% to 75%, from 40% to 75%, from 45% to 75%, from 50% to 75%, from 55% to 75%, from 60% to 75%, from 65% to 75%, from 70% to 75%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 20% to 65%, from 25% to 65%, from 30% to 65%, from 35% to 65%, from 40% to 65%, from 45% to 65%, from 50% to 65%, from 55% to 65%, from 60% to 65%, from 20% to 60%, from 25% to 60%, from 30% to 60%, from 35% to 60%, from 40% to 60%, from 45% to 60%, from 50% to 60%, from 55% to 60%, from 20% to 55%, from 25% to 55%, from 30% to 55%, from 35% to 55%, from 40% to 55%, from 45% to 55%, from 50% to 55%, from 20% to 50%, from 25% to 50%, from 30% to 50%, from 35% to 50%, from 40% to 50%, from 45% to 50%, from 20% to 45%, from 25% to 45%, from 30% to 45%, from 35% to 45%, from 40% to 45%, from 20% to 40%, from 25% to 40%, from 30% to 40%, from 35% to 40%, from 20% to 35%, from 25% to 35%, from 30% to 35%, from 20% to 30%, from 25% to 30%, or from 20% to 25%. In some embodiments of the methods provided herein including in this paragraph, the PRI is the MRS. In certain embodiments of the methods provided herein including in this paragraph the PRI is the RPS. In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase in TL1A protein expression in a sample obtained from a subject as compared to a reference level of TL1A protein expression (e.g., level in a tissue not affected by IBD) with a specificity of at least 40%. The other embodiments described in this paragraph are provided for the various methods described herein including in Sections 2, 5.2 (e.g. the preceding paragraphs), and 7. In some embodiments, the PRI above the cutoff predicts an increase in TL1A protein expression in a sample obtained from a subject as compared to a reference level of TL1A protein expression (e.g., level in a tissue not affected by IBD) with a specificity of at least 45%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 50%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 55%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 60%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 65%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 70%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 75%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 80%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 85%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 90%. In some embodiments, the PRI above the cutoff predicts an increase in TL1A protein expression in a sample obtained from a subject as compared to a reference level of TL1A protein expression (e.g., level in a tissue not affected by IBD) with a specificity of at least 95%. In some embodiments, the PRI above the cutoff predicts an increase in TL1A protein expression in a sample obtained from a subject as compared to a reference level of TL1A protein expression (e.g., level in a tissue not affected by IBD) with a specificity of about 40%. In some embodiments, the PRI above the cutoff predicts an increase in TL1A protein expression in a sample obtained from a subject as compared to a reference level of TL1A protein expression (e.g., level in a tissue not affected by IBD) with a specificity of about 45%. In some embodiments, the PRI above the cutoff predicts an increase in TL1A protein expression in a sample obtained from a subject as compared to a reference level of TL1A protein expression (e.g., level in a tissue not affected by IBD) with a specificity of about 50%. In some embodiments, the PRI above the cutoff predicts an increase in TL1A protein expression in a sample obtained from a subject as compared to a reference level of TL1A protein expression (e.g., level in a tissue not affected by IBD) with a specificity of about 55%. In some embodiments, the PRI above the cutoff predicts an increase in TL1A protein expression in a sample obtained from a subject as compared to a reference level of TL1A protein expression (e.g., level in a tissue not affected by IBD) with a specificity of about 60%. In some embodiments, the PRI above the cutoff predicts an increase in TL1A protein expression in a sample obtained from a subject as compared to a reference level of TL1A protein expression (e.g., level in a tissue not affected by IBD) with a specificity of about 65%. In some embodiments, the PRI above the cutoff predicts an increase in TL1A protein expression in a sample obtained from a subject as compared to a reference level of TL1A protein expression (e.g., level in a tissue not affected by IBD) with a specificity of about 70%. In some embodiments, the PRI above the cutoff predicts an increase in TL1A protein expression in a sample obtained from a subject as compared to a reference level of TL1A protein expression (e.g., level in a tissue not affected by IBD) with a specificity of about 75%. In some embodiments, the PRI above the cutoff predicts an increase in TL1A protein expression in a sample obtained from a subject as compared to a reference level of TL1A protein expression (e.g., level in a tissue not affected by IBD) with a specificity of about 80%. In some embodiments, the PRI above the cutoff predicts an increase in TL1A protein expression in a sample obtained from a subject as compared to a reference level of TL1A protein expression (e.g., level in a tissue not affected by IBD) with a specificity of about 85%. In some embodiments, the PRI above the cutoff predicts an increase in TL1A protein expression in a sample obtained from a subject as compared to a reference level of TL1A protein expression (e.g., level in a tissue not affected by IBD) with a specificity of about 90%. In some embodiments, the PRI above the cutoff predicts an increase in TL1A protein expression in a sample obtained from a subject as compared to a reference level of TL1A protein expression (e.g., level in a tissue not affected by IBD) with a specificity of about 95%. In some embodiments, the PRI above the cutoff predicts an increase in TL1A protein expression in a sample obtained from a subject as compared to a reference level of TL1A protein expression (e.g., level in a tissue not affected by IBD) with a specificity from 50% to 95%, from 55% to 95%, from 60% to 95%, from 65% to 95%, from 70% to 95%, from 75% to 95%, from 80% to 95%, from 85% to 95%, from 90% to 95%, from 50% to 90%, from 55% to 90%, from 60% to 90%, from 65% to 90%, from 70% to 90%, from 75% to 90%, from 80% to 90%, from 85% to 90%, from 50% to 85%, from 55% to 85%, from 60% to 85%, from 65% to 85%, from 70% to 85%, from 75% to 85%, from 80% to 85%, from 50% to 80%, from 55% to 80%, from 60% to 80%, from 65% to 80%, from 70% to 80%, from 75% to 80%, from 50% to 75%, from 55% to 75%, from 60% to 75%, from 65% to 75%, from 70% to 75%, from 50% to 70%, from 55% to 70%, from 60% to 70%, from 65% to 70%, from 50% to 65%, from 55% to 65%, from 60% to 65%, from 50% to 60%, from 55% to 60%, or from 50% to 55%. In some embodiments of the methods provided herein including in this paragraph, the PRI is the MRS. In certain embodiments of the methods provided herein including in this paragraph the PRI is the RPS. In some embodiments of the methods provided herein including in this paragraph, the subject is a subject having IBD. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff, or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

In various embodiments of the methods provided herein including in Sections 2, 5.2 (e.g. the preceding paragraphs), and 7, the PRI above the cutoff predicts an increase in TL1A protein expression in a sample obtained from a subject as compared to a reference level of TL1A protein expression (e.g., level in a tissue not affected by IBD) with an accuracy of at least 50%. The other embodiments described in this paragraph are provided for the various methods described herein including in Sections 2, 5.2 (e.g. the preceding paragraphs), and 7. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 55%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 60%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 65%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 70%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 75%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 80%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 85%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 90%. In some embodiments, the PRI above the cutoff predicts an increase in TL1A protein expression in a sample obtained from a subject as compared to a reference level of TL1A protein expression (e.g., level in a tissue not affected by IBD) with an accuracy of at least 95%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 50%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 55%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 60%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 65%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 70%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 75%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 80%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 85%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 90%. In some embodiments, the PRI above the cutoff predicts an increase in TL1A protein expression in a sample obtained from a subject as compared to a reference level of TL1A protein expression (e.g., level in a tissue not affected by IBD) with an accuracy of about 95%. In some embodiments, the PRI above the cutoff predicts an increase in TL1A protein expression in a sample obtained from a subject as compared to a reference level of TL1A protein expression (e.g., level in a tissue not affected by IBD) with an accuracy of from 60% to 95%, from 65% to 95%, from 70% to 95%, from 75% to 95%, from 80% to 95%, from 85% to 95%, from 90% to 95%, from 60% to 90%, from 65% to 90%, from 70% to 90%, from 75% to 90%, from 80% to 90%, from 85% to 90%, from 60% to 85%, from 65% to 85%, from 70% to 85%, from 75% to 85%, from 80% to 85%, from 60% to 80%, from 65% to 80%, from 70% to 80%, from 75% to 80%, from 60% to 75%, from 65% to 75%, from 70% to 75%, from 60% to 70%, from 65% to 70%, or from 60% to 65%. In some embodiments of the methods provided herein including in this paragraph, the PRI is the MRS. In certain embodiments of the methods provided herein including in this paragraph the PRI is the RPS. In some embodiments of the methods provided herein including in this paragraph, the subject is a subject having 113D. In some embodiments of the methods provided herein including in this paragraph, the subject is a subject having UC. In some embodiments of the methods provided herein including in this paragraph, the subject is a subject having CD. Furthermore, for each embodiment described in this paragraph, also provided is a corresponding embodiment wherein the phrase “the PRI above the cutoff predicts” is substituted by the phrase “the cutoff is such that the comparison of the PRI to the cutoff is predictive of.” In some further embodiments of the previous clause, the comparison of the PRI to the cutoff is determined according to (1) or (2) as follows: (1) if the PRI has a positive correlation with RPS, then the comparison of the PRI to the cutoff is based on the PRI above the cutoff; or (2) if the PRI has a negative correlation with RPS, then the comparison of the PRI to the cutoff is based on the PRI below the cutoff. Similarly, in some further embodiments, the comparison of the PRI to the cutoff is determined according to (1) or (2) as follows: (1) if the PRI has a positive correlation with MRS, then the comparison of the PRI to the cutoff is based on the PRI above the cutoff; or (2) if the PRI has a negative correlation with MRS, then the comparison of the PRI to the cutoff is based on the PRI below the cutoff.

The disclosure provides that for the embodiments of the preceding 23 paragraphs, when PRI above a cutoff achieves a certain metrics such as PPV, specificity, sensitivity, NPV, positive rate, or accuracy, a cutoff is determined and provided such that the PRI above the cutoff can achieve the metrics recited in the preceding 23 paragraphs. Accordingly, In some embodiments of the methods provided herein including Sections 2, 5.2 (e.g. the preceding paragraphs such as the preceding 23 paragraphs), and 7, the cutoff is such that the PRI above the cutoff achieve any of the PPV, specificity, sensitivity, NPV, positive rate, or accuracy described in the preceding paragraphs such as the preceding 23 paragraphs. Specifically, in some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with a positive predictive value of at least about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%. In some embodiments of the methods provided herein, including in Sections 2, 5.2 (e.g. the preceding paragraphs such as the preceding 23 paragraphs), and 7, the cutoff is such that the PRI above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with a positive predictive value of at least about 25%, 26%, 27%, 28% 29%, 30%, 31%, 32%, 33%, 34%, 35%, 40%, 45%, 50%, 51%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with a positive predictive value of at least about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with a specificity of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with a specificity of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with a negative predictive value of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with a negative predictive value of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with a sensitivity of at least about 20%, 25%, 30%, 35%, 40%, 45%, 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with a sensitivity of at least about 20%, 25%, 30%, 35%, 40%, 45%, 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with a positive rate of at least about 10%, 15%, 20%, 25%, 30%, 35%, 40%, 45%, 50%, 55%, 60%, 65%, 70%, or 75%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with a positive rate of at least about 10%, 15%, 20%, 25%, 30%, 35%, 40%, 45%, 50%, 55%, 60%, 65%, 70%, or 75%. In some embodiments of the methods provided herein, including in Sections 2, 5.2 (e.g. the preceding paragraphs such as the preceding 23 paragraphs), and 7, the cutoff is such that the PRI above the cutoff is predictive of an increase of one or more IBD enriched cell types with an accuracy of at least about 50%, 55%, 60%, 65%, 70%, 75%, 80%, 85%, 90%, or 95%. In some embodiments of the methods provided herein, including in Sections 2, 5.2 (e.g. the preceding paragraphs such as the preceding 23 paragraphs), and 7, the cutoff is such that the PRI above the cutoff is predictive of a decrease of one or more IBD depleted cell types with an accuracy of at least about 50%, 55%, 60%, 65%, 70%, 75%, 80%, 85%, 90%, or 95%. In some embodiments of the methods provided herein, including in this paragraph, the cutoff can be determined as described in Sections 5.2 and 7.20.4.

The term “IBD enriched cell type” refers to cell types (i) that are increased in a diseased tissue of IBD patients over non-diseased control tissues and (ii) the increase of which are associated with TL1A-mediated inflammatory, a fibrotic, or a fibrostenotic disease or condition, such as IBD, UC, and CD and therefore the increase of which are predictive of enhanced probability of responding to treatment with an inhibitor of TL1A activity or expression. Such IBD enriched cell types are increased in IBD related tissues, the IBD enriched cell types' increases are associated with TL1A disease pathology, and the IBD enriched cell types can be identified by analyzing the up- or down-regulation of various cells in IBD diseased tissues (via single cell analyses or deconvoluting bulk cell data into single cells) as described in Section 7.20. In some examples, IBD enriched cell types include activated fibroblasts, monocyte-derived dendritic cells (moDCs), and CD36+ endothelial cells as established in in Section 7.20.

The term “IBD depleted cell type” refers to cell types (i) that are decreased in a diseased tissue of IBD patients over non-diseased control tissues and (ii) decrease of which are associated with TL1A-mediated inflammatory, a fibrotic, or a fibrostenotic disease or condition, such as IBD, UC, and CD, and therefore decrease of which are predictive of enhanced probability of responding to treatment with an inhibitor of TL1A activity or expression. Such IBD depleted cell types are decreased in IBD related tissues, IBD depleted cell types' decreases are associated with TL1A disease pathology, and the IBD depleted cell types can be identified by analyzing the up- or down-regulation of various cells in IBD diseased tissues (via single cell analyses or deconvoluting bulk cell data into single cells) as described in Section 7.20. In some examples, IBD depleted cell types include Tuft cells and BEST4+ epithelial cells as established in in Section 7.20.

In various embodiments of the methods provided herein including in Sections 2, 5.2 (e.g. the preceding paragraphs), and 7, the IBD enriched cell types comprise activated fibroblasts. In some embodiments of the methods provided herein including in Sections 2, 5.2 (e.g. the preceding paragraphs), and 7, the IBD enriched cell types comprise moDCs. In certain embodiments of the methods provided herein including in Sections 2, 5.2 (e.g. the preceding paragraphs), and 7, the IBD enriched cell types comprise CD36+ endothelial cells. In some embodiments of the methods provided herein including in Sections 2, 5.2 (e.g. the preceding paragraphs), and 7, the IBD enriched cell types comprise enterocytes and clonocytes (entero_clonocyte in Table 31). In some embodiments of the methods provided herein including in Sections 2, 5.2 (e.g. the preceding paragraphs), and 7, the IBD enriched cell types comprise enteroendocrine cells (EEC in Table 31). In some embodiments of the methods provided herein including in Sections 2, 5.2 (e.g. the preceding paragraphs), and 7, the IBD enriched cell types comprise goblet cells. In some embodiments of the methods provided herein including in Sections 2, 5.2 (e.g. the preceding paragraphs), and 7, the IBD enriched cell types comprise IgG plasma cells. In some embodiments of the methods provided herein including in Sections 2, 5.2 (e.g. the preceding paragraphs), and 7, the IBD enriched cell types comprise Paneth cells. In some embodiments of the methods provided herein including in Sections 2, 5.2 (e.g. the preceding paragraphs), and 7, the IBD enriched cell types comprise resident.macrophages. In some embodiments of the methods provided herein including in Sections 2, 5.2 (e.g. the preceding paragraphs), and 7, the IBD enriched cell types comprise transit-amplifying (TA) progenitor cells. In some embodiments of the methods provided herein including in Sections 2, 5.2 (e.g. the preceding paragraphs), and 7, the IBD enriched cell types comprise highly activated T cells. In some embodiments of the methods provided herein including in Sections 2, 5.2 (e.g. the preceding paragraphs), and 7, the IBD enriched cell types comprise lymphatic epithelial cells (lymphatics in Table 31). In some embodiments of the methods provided herein including in Sections 2, 5.2 (e.g. the preceding paragraphs), and 7, the IBD enriched cell types comprise microfold cells. In some embodiments of the methods provided herein including in Sections 2, 5.2 (e.g. the preceding paragraphs), and 7, the IBD enriched cell types comprise myofibroblasts.

In some embodiments of the methods provided herein including in Sections 2, 5.2 (e.g. the preceding paragraphs), and 7, the IBD enriched cell types comprise any one selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any two selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any three selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any four selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any five selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any six selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any seven selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any eight selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any nine selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any ten selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any eleven selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any twelve selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any thirteen selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise all fourteen selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts. In some embodiments of the methods provided herein including in Sections 2, 5.2 (e.g. the preceding paragraphs), and 7, the IBD enriched cell types comprise any one or more (e.g. any 1 to 14) selected from the group consisting of activated fibroblasts, moDCs, CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts.

In certain embodiments of the methods provided herein including in Sections 2, 5.2 (e.g. the preceding paragraphs), and 7, the IBD depleted cell types comprise Tuft cells. In some embodiments of the methods provided herein including in Sections 2, 5.2 (e.g. the preceding paragraphs), and 7, the IBD depleted cell types comprise BEST4+ epithelial cells. In some embodiments of the methods provided herein including in Sections 2, 5.2 (e.g. the preceding paragraphs), and 7, the IBD depleted cell types comprise both Tuft cells and BEST4+ epithelial cells

As used herein, the term “positive therapeutic response” is intended to mean improvement of one or more aspects of the symptoms of the inflammatory, fibrotic, or fibrostenotic disease or condition in a subject after treatment with the inhibitor of TL1A activity or expression provided herein, as determined by clinical parameters for such disease or condition. As is clear from the description herein, the inflammatory, fibrotic, or fibrostenotic disease or condition can be inflammatory bowel disease such as Crohn's disease or ulcerative colitis. In the context of ulcerative colitis, such improvement of the disease symptoms can be measured by Mayo Score (based on stool frequency, rectal bleeding, the endoscopic appearance of the mucosa (endoscopic score), and a Physician's Global Assessment (PGA), each of which were scored on a scale from 0 to 3, giving a Mayo Score of 0-12) or Modified Mayo Score (based on stool frequency, rectal bleeding, and the endoscopic appearance of the mucosa (endoscopic score), each of which were scored on a scale from 0 to 3, giving a Modified Mayo Score of 0-9), as known and practiced in the field. For example, in one embodiment, the positive therapeutic response in UC can be an improvement of the 3-component Modified Mayo Score, as determined by a reduction from baseline by ≥2 points and ≥30% in the 3-component Modified Mayo Score, accompanied by a reduction≥1 in rectal bleeding subscore or absolute rectal bleeding subscore≤1. In the context of Crohn's disease, such improvement of the disease symptoms can be measured by Crohn's disease activity index (CDAI), simple endoscopy score for Crohn's disease (SES-CD, e.g. as further described in Gastrointest Endosc 2004; 60:505-512), or both CDAI and SES-CD, as known and practiced in the field. For example, in one embodiment, the positive therapeutic response can be an improvement of the CDAI, as determined by a reduction from baseline by ≥100 in CDAI. In another example, the positive therapeutic response can be a clinical remission, as defined below. In another example, the positive therapeutic response can be an endoscopic improvement, as defined below.

As used herein, the term “clinical remission” is intended to mean (i) the symptoms of the inflammatory, fibrotic, or fibrostenotic disease or condition in a subject have improved to the extent that the symptoms are absent, close to be absent, or moving close towards disappearance, or (ii) the inflammatory, fibrotic, or fibrostenotic disease or condition in the subject becomes a mild or less than a mild disease, after treatment with the inhibitor of TL1A activity or expression provided herein, as determined by clinical parameters for such disease or condition. As is clear from the description herein, the inflammatory, fibrotic, or fibrostenotic disease or condition can be inflammatory bowel disease such as Crohn's disease or ulcerative colitis. In the context of ulcerative colitis, the clinical remission can be measured by Mayo Score or Modified Mayo Score, each as described above in the definition of “positive therapeutic response”. For example, in one embodiment, the clinical remission in UC can be determined based on the 3-component Modified Mayo Score as an endoscopic subscore of 0 or 1, rectal bleeding subscore of 0, and stool frequency subscore of 0 or 1 and not greater than baseline. In the context of Crohn's disease, such improvement of the disease symptoms can be measured by CDAI, SES-CD, or both CDAI and SES-CD, as known and practiced in the field. For example, in one embodiment, the clinical remission in CD can be determined based on the CDAI, as CDAI<150.

As used herein, the term “endoscopic improvement” is intended to mean (i) the inflammation in the mucosa of the digestive tract of the subject is absent, close to be absent, or moving close towards disappearance, or (ii) substantial reduction (≥50% reduction) of the inflammation in the mucosa of the digestive tract in a subject, after treatment with the inhibitor of TL1A activity or expression provided herein, as determined by a colonoscopy, a sigmoidoscopy, or other applicable endoscopy. As is clear from the description herein, the subject can be a subject affected by inflammatory, fibrotic, or fibrostenotic disease or condition, for example, inflammatory bowel disease such as Crohn's disease or ulcerative colitis. In the context of ulcerative colitis, the endoscopic improvement can be measured by the endoscopy subscore that is used as a component of the Mayo Score or Modified Mayo Score. For example, in one embodiment, the endoscopic improvement in UC can be determined based on endoscopy subscore≤1 with no friability. In the context of Crohn's disease, such endoscopic improvement can be measured by SES-CD, as known and practiced in the field. For example, in one embodiment, the endoscopic improvement in CD can be determined based on decrease in simple endoscopy score for Crohn's disease (SES-CD) 50% from baseline.

In some embodiments of the methods provided herein including in Sections 2, 5.2 (e.g. the preceding paragraphs), and 7, the positive therapeutic response is clinical remission. In some embodiments of the methods provided herein including in Sections 2, 5.2 (e.g. the preceding paragraphs), and 7, the positive therapeutic response is endoscopic improvement. In some embodiments of the methods provided herein including in Sections 2, 5.2 (e.g. the preceding paragraphs), and 7, the positive therapeutic response is histologic remission (determined by Geboes score≤3.1, as known and practiced in the field of IBD). In some embodiments of the methods provided herein including in Sections 2, 5.2 (e.g. the preceding paragraphs), and 7, the positive therapeutic response is histologic-endoscopic mucosal improvement (determined by Geboes score≤3.1 and endoscopy subscore≤1 with no friability). In some embodiments of the methods provided herein including in Sections 2, 5.2 (e.g. the preceding paragraphs), and 7, the positive therapeutic response is histologic improvement (determined by Geboes score≤3.1). In some embodiments of the methods provided herein including in Sections 2, 5.2 (e.g. the preceding paragraphs), and 7, the positive therapeutic response is mucosal healing (determined by Geboes score≤2B.1 and endoscopy subscore of ≤1). In some embodiments of the methods provided herein including in Sections 2, 5.2 (e.g. the preceding paragraphs), and 7, the positive therapeutic response is inflammatory bowel disease questionnaire (IBDQ) response, as determined by 16-point increase from baseline.

In some embodiments of the methods provided herein including in Sections 2, 5.2 (e.g. the preceding paragraphs), and 7, the positive therapeutic response is determined at the end of induction dose regimen. In some embodiments of the methods provided herein including in Sections 2, 5.2 (e.g. the preceding paragraphs), and 7, the positive therapeutic response is determined at week 12.

The disclosure specifically provides that the various embodiments of positive predictive value as provided herein (including in Sections 2, 5.2 (e.g. the preceding paragraphs), and 7) can be combined with the various embodiments of specificity as provided herein (Sections 2, 5.2 (e.g. the preceding paragraphs), and 7) in various embodiments of the methods, kit, and compositions as provided herein (Sections 2, 5.2 (e.g. the preceding paragraphs), and 7).

The disclosure also specifically provides that the various embodiments of positive predictive value as provided herein (including in Sections 2, 5.2 (e.g. the preceding paragraphs), and 7) can be combined with the various embodiments of the positive rate of PRI above the cutoff as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure further specifically provides that the various embodiments of positive predictive value as provided herein (including in Sections 2, 5.2 (e.g. the preceding paragraphs), and 7) can be combined with the various embodiments of sensitivity as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure additionally provides that the various embodiments of positive predictive value as provided herein (including in Sections 2, 5.2 (e.g. the preceding paragraphs), and 7) can be combined with the various embodiments of negative predictive value as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure also provides that the various embodiments of negative predictive value as provided herein (including in Sections 2, 5.2 (e.g. the preceding paragraphs), and 7) can be combined with the various embodiments of specificity as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure specifically provides that the various embodiments of negative predictive value as provided herein (including in Sections 2, 5.2 (e.g. the preceding paragraphs), and 7) can be combined with the various embodiments of sensitivity as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure further specifically provides that the various embodiments of sensitivity as provided herein (including in Sections 2, 5.2 (e.g. the preceding paragraphs), and 7) can be combined with the various embodiments of specificity as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure also specifically provides that the various embodiments of sensitivity as provided herein (including in Sections 2, 5.2 (e.g. the preceding paragraphs), and 7) can be combined with the various embodiments of the positive rate of PRI above the cutoff as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure additionally provides that the various embodiments of specificity as provided herein (including in Sections 2, 5.2 (e.g. the preceding paragraphs), and 7) can be combined with the various embodiments of the positive rate of PRI above the cutoff as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

The disclosure specifically provides that the various embodiments of negative predictive value as provided herein (including in Sections 2, 5.2 (e.g. the preceding paragraphs), and 7) can be combined with the various embodiments of the positive rate of PRI above the cutoff as provided herein (including in Sections 2, 5.2 (e.g. the preceding paragraphs), and 7) in various embodiments of the methods, kit, and compositions as provided herein (including in Sections 2, 5.2 (e.g. the preceding paragraphs), 5.5, 5.6, 5.7, 5.8, and 7).

Additionally, the disclosure provides that positive predictive value, negative predictive value, sensitivity, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7).

In some embodiments, the disclosure provides that negative predictive value, sensitivity, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, sensitivity, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, negative predictive value, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, negative predictive value, sensitivity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, negative predictive value, sensitivity, and/or specificity, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7).

In some embodiments, the disclosure provides that positive predictive value, negative predictive value, and/or sensitivity, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, negative predictive value, and/or specificity, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, negative predictive value, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, sensitivity, and/or specificity, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, sensitivity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that positive predictive value, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that negative predictive value, sensitivity, and/or specificity, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that negative predictive value, sensitivity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that negative predictive value, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7). In some embodiments, the disclosure provides that sensitivity, specificity, and/or positive rate of PRI above the cutoff, each as provided herein (including in Sections 2, 5.2 (e.g. the preceding paragraphs), and 7), can be combined in various combinations in various embodiments of the methods, kit, and compositions as provided herein (including in Sections 2, 5.2 (e.g. the preceding paragraphs), 5.5, 5.6, 5.7, 5.8, and 7).

In some embodiments of the methods provided herein including in Sections 2, 5.2 (e.g. the preceding paragraphs), and 7, the combination of polymorphisms comprises one or more polymorphisms selected from Table 27, or a proxy polymorphism in linkage disequilibrium therewith as determined with an R² of at least 0.85, or a combination thereof. In certain embodiments of the methods provided herein including in Sections 2, 5.2 (e.g. the preceding paragraphs), and 7, the combination of polymorphisms comprises one polymorphism selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises two polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises three polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises four polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises five polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises six polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises seven polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises eight polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises nine polymorphisms selected from Table 27, or a proxy polymorphism in linkage disequilibrium therewith as determined with an R² of at least 0.85, or a combination thereof. In some embodiments of the methods provided herein including in Sections 2, 5.2 (e.g. the preceding paragraphs), and 7, the combination of polymorphisms comprises ten polymorphisms selected from Table 27, or a proxy polymorphism in linkage disequilibrium therewith as determined with an R² of at least 0.85, or a combination thereof. In certain embodiments of the methods provided in this paragraph, any polymorphism from Table 27 used in the combination of polymorphisms of the methods can be replaced with a proxy polymorphism in linkage disequilibrium therewith as determined with an R² of at least 0.85. Similarly, any 1 to 10 polymorphisms (each an original polymorphism) from Table 27 used in the combination of polymorphisms of the methods can be replaced with corresponding proxy polymorphisms, wherein each proxy polymorphism is in linkage disequilibrium with the original polymorphism as determined with an R² of at least 0.85.

In some embodiments of the various methods provided herein, including in Sections 2, 5.2, and 7, the combination of polymorphisms in the methods comprises a combination described in columns 1 and 2 of Table 31. In some embodiments of the various methods provided herein, including in Sections 2, 5.2, and 7, the combination of polymorphisms in the methods comprises a combination described in columns 1 and 2 of Table 31, and the mathematical representation of the polymorphism in the methods can be any one of the mathematical representations described in Table 28. As is clear from the description of Table 31, each row in columns 1 and 2 of Table 31 describes a specific combination of β coefficients and the corresponding SNPs. Accordingly, in some embodiments of the various methods provided herein including in Sections 2, 5.2 (including in this paragraph), and 7, the combination of polymorphisms in the methods comprises a combination described in any one row of columns 1 and 2 of Table 31. More specifically, in some embodiments of the various methods provided herein including in Sections 2, 5.2 (including in this paragraph), and 7, the combination of polymorphisms in the methods comprises a combination described in row x of columns 1 and 2 of Table 31, and wherein x is any number between 2 to 1374. In certain embodiments of the methods provided in this paragraph, any polymorphism from Table 31 used in the combination of polymorphisms of the methods can be replaced with a proxy polymorphism in linkage disequilibrium therewith as determined with an R² of at least 0.85. Similarly, any 1 to 10 polymorphisms (each an original polymorphism) from Table 31 used in the combination of polymorphisms of the methods can be replaced with corresponding proxy polymorphisms, wherein each proxy polymorphism is in linkage disequilibrium with the original polymorphism as determined with an R² of at least 0.85.

For various embodiments of the methods provided herein, including in Sections 2, 5.2 (including in this paragraph), and 7, the method comprises various elements or combinations of elements provided herein, for example, as described in the following: further embodiments of the therapeutic agent including anti-TL1A therapeutic agents and anti-TL1A antibodies (e.g., anti-TL1A embodiments with exemplary CDRs, framework sequences, constant region sequences, Fc mutations, variable regions, Fc regions, and other properties) are provided in Sections 2, 5.4, and 7; assays for detecting the genotypes or polymorphisms are provided in Sections 2, 5.3, 5.8, 5.9.5, and 7; assays for validating the genotypes or genetic polymorphisms for the methods are provided in in Sections 2, 5.3, 5.8, and 7; methods of preparing the samples for detecting the genotypes or polymorphisms are provided in Section 5.9 (including Sections 5.9.1 to 5.9.4); genotypes, genetic polymorphisms, or other criteria used for selecting patients or subjects for the methods are provided in Section 2, 5.1, 5.2 (including in this paragraph), and 7; methods for selecting the patients for the treatment is provided in Section 2, 5.1, 5.2 (including in this paragraph), and 7; pharmaceutical compositions for the anti-TL1A antibodies are described and provided in Sections 2, 5.4, 5.5, and 7; the step of, the software for, and the system for interpreting the genotype, the combination of genotypes, the polymorphism, or the combination of polymorphisms are described in Section 2, 5.2 (including in this paragraph), 5.8, and 7; and/or further specific and validated embodiments for the therapeutic agents against TL1A and the methods of using the same are provided in Sections 2, 5.4 (e.g. Section 5.4.1.5), and 7. As such, the disclosure provides the various combinations of the therapeutic agent against TL1A, the pharmaceutical compositions of such therapeutic agent against TL1A, the methods of generating the therapeutic agent against TL1A, the methods of assaying the genotypes and polymorphisms, the methods of preparing the sample, the methods of interpreting the genotype, the combination of genotypes, the polymorphism, or the combination of polymorphisms, and/or the methods of using the therapeutic agent against TL1A for treatment.

As is clear from the above description, the patients or subjects are selected with the methods of selecting patients or subjects as provided herein including in Sections 2, 5.2 (including in the preceding paragraphs), and 7, and then subject to the various methods of treatment as provided herein including in Sections 2, 5.2 (including in the preceding paragraphs), and 7.

For various embodiments of the methods provided herein, including in Sections 2, 5.2 (including in the preceding paragraphs), and 7, the inflammatory disease comprises or is inflammatory bowel disease.

For various embodiments of the methods provided herein, including in Sections 2, 5.2 (including in the preceding paragraphs), and 7, the inflammatory disease comprises or is Crohn's disease. In some embodiments, the IBD comprises or is Crohn's disease.

For various embodiments of the methods provided herein, including in Sections 2, 5.2 (including in the preceding paragraphs), and 7, the Crohn's disease comprises or consists of ileal, ileocolonic, or colonic Crohn's disease.

For various embodiments of the methods provided herein, including in Sections 2, 5.2 (including in the preceding paragraphs), and 7, the inflammatory disease comprises or is ulcerative colitis (UC). In some embodiments, the IBD comprises or is UC.

For various embodiments of the methods provided herein, including in Sections 2, 5.2 (including in the preceding paragraphs), and 7, the UC is medically refractory UC.

For various embodiments of the methods provided herein, including in Sections 2, 5.2 (including in the preceding paragraphs), and 7, the sample is a biological sample.

For various embodiments of the methods, kit, systems, processes, or compositions provided herein, including in Sections 2, 5 (including in the paragraphs of Section 5.2), and 7, the corresponding alternative methods, kit, systems, processes, or compositions are also provided herein in which “comprising” is replaced by “consisting of.” In various embodiments of the methods, kit, systems, processes, or compositions provided herein, including in Sections 2, 5 (including in the paragraphs of Section 5.2), and 7, the corresponding alternative methods, kit, systems, processes, or compositions are also provided herein in which “comprise(s)” is replaced by “consist(s) of.”

Additionally, the disclosure provides various assays for determining or detecting the genotypes, combinations of genotypes, polymorphisms, or combinations of polymorphisms. As such, in various embodiments of the methods provided herein including in Sections 2, 5 (including in the paragraphs of Section 5.2), and 7, determining or detecting the genotypes, combinations of genotypes, polymorphisms, or combinations of polymorphisms comprises or consists of assaying for the genotypes, combinations of genotypes, polymorphisms, or combinations of polymorphisms via any assays as described in Sections 2, 5.3, 5.9.5, and 7. In some embodiments of the methods provided herein including in Sections 2, 5 (including in the paragraphs of Section 5.2), and 7, determining or detecting the genotypes, combinations of genotypes, polymorphisms, or combinations of polymorphisms comprises or consists of determining or detecting the genotypes, combinations of genotypes, polymorphisms, or combinations of polymorphisms via any assays as described in Sections 2, 5.3, 5.9.5, and 7. In some embodiments of the methods provided herein including in Sections 2, 5 (including in the paragraphs of Section 5.2), and 7, determining or detecting the genotypes, combinations of genotypes, polymorphisms, or combinations of polymorphisms comprises or consists of determining or detecting the genotypes, combinations of genotypes, polymorphisms, or combinations of polymorphisms via any or any combination of method steps as described in Sections 2, 5.3, 5.9.5, and 7. Alternatively, in various embodiments of the methods provided herein including in Sections 2, 5 (including in the paragraphs of Section 5.2), and 7, the method further comprises assaying for the genotypes, combinations of genotypes, polymorphisms, or combinations of polymorphisms via any assays as described in Sections 2, 5.3, 5.9.5, and 7. In certain embodiments of the methods provided herein including in Sections 2, 5 (including in the paragraphs of Section 5.2), and 7, the method further comprises assaying for the genotypes, combinations of genotypes, polymorphisms, or combinations of polymorphisms via any or any combination of method steps as described in Sections 2, 5.3, 5.9.5, and 7.

Specifically, for various embodiments of the methods provided herein, including in Sections 2, 5 (including in the paragraphs of Section 5.2), and 7, the anti-TL1A antibody or antigen binding fragment thereof used in the methods is any one selected from the anti-TL1A antibodies and antigen binding fragments described in Section 5.4. Additionally, for various embodiments of the methods provided herein, including in Sections 2, 5 (including in the paragraphs of Section 5.2), and 7, the inhibitor of TL1A activity or expression is any one selected from the inhibitors of TL1A activity or expression described in Section 5.4. In one specific embodiment of the methods provided herein, including in Sections 2, 5 (including in the paragraphs of Section 5.2), and 7, the anti-TL1A antibody or antigen binding fragment thereof used in the method comprises or consists of any antibody or antigen binding fragment selected from those provided in Section 5.4. In another specific embodiment of the methods provided herein, including in Sections 2, 5 (including in the paragraphs of Section 5.2), and 7, the anti-TL1A antibody or antigen binding fragment thereof used in the method is selected from those listed in Table 15 and Table 20. In one specific embodiment of the methods provided herein, including in Sections 2, 5 (including in the paragraphs of Section 5.2), and 7, the subject in the method has a genotype selected from the genotypes described in Section 5.1. In another specific embodiment of the methods provided herein, including in Sections 2, 5 (including in the paragraphs of Section 5.2), and 7, the subject in the method has a combination of genotypes selected from the combinations of genotypes described in Section 5.1. In yet another specific embodiment of the methods provided herein, including in Sections 2, 5 (including in the paragraphs of Section 5.2), and 7, the subject in the method has a combination of polymorphisms selected from the combination of polymorphisms described in Section 5.1. In one specific embodiment of the methods provided herein, including in Sections 2, 5 (including in the paragraphs of Section 5.2), and 7, the subject in the method has a genotype selected from the genotypes described in Section 5.1 and the anti-TL1A antibody or antigen binding fragment thereof used in the methods is any one selected from the anti-TL1A antibodies and antigen binding fragments described in Section 5.4. In another specific embodiment of the methods provided herein, including in Sections 2, 5 (including in the paragraphs of Section 5.2), and 7, the subject in the method has a combination of genotypes selected from the combinations of genotypes described in Section 5.1 and the anti-TL1A antibody or antigen binding fragment thereof used in the methods is any one selected from the anti-TL1A antibodies and antigen binding fragments described in Section 5.4. In yet another specific embodiment of the methods provided herein, including in Sections 2, 5 (including in the paragraphs of Section 5.2), and 7, the subject in the method has a combination of polymorphisms selected from the combination of polymorphisms described in Section 5.1 and the anti-TL1A antibody or antigen binding fragment thereof used in the methods is any one selected from the anti-TL1A antibodies and antigen binding fragments described in Section 5.4.

In one specific embodiment of the methods provided herein, including in Sections 2, 5 (including in the paragraphs of Section 5.2), and 7, the subject in the method has a genotype selected from the genotypes described in Section 5.1. In another specific embodiment of the methods provided herein, including in Sections 2, 5 (including in the paragraphs of Section 5.2), and 7, the subject in the method has a combination of genotypes selected from the combinations of genotypes described in Section 5.1. In yet another specific embodiment of the methods provided herein, including in Sections 2, 5 (including in the paragraphs of Section 5.2), and 7, the subject in the method has a combination of polymorphisms selected from the combination of polymorphisms described in Section 5.1.

In one embodiment of the methods provided herein, including in Sections 2, 5 (including in the paragraphs of Section 5.2), and 7, the MRS is calculated from a combination of genotypes provided herein (e.g. in Sections 2, 5.1, and 7), wherein the MRS above a cutoff is predictive of (i) an increase in a level of TNFSF15 (TL1A) protein expression in a sample obtained from a subject or patient, as compared to a reference level of TNFSF15 (TL1A) protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. In one embodiment of the methods provided herein, including in Sections 2, 5 (including in the paragraphs of Section 5.2), and 7, the RPS is calculated from a combination of genotypes provided herein (e.g. in Sections 2, 5.1, and 7), wherein the RPS above a cutoff is predictive of (i) an increase in a level of TNFSF15 (TL1A) protein expression in a sample obtained from a subject or patient, as compared to a reference level of TNFSF15 (TL1A) protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. In one embodiment of the methods provided herein, including in Sections 2, 5 (including in the paragraphs of Section 5.2), and 7, the PRI is calculated from a combination of genotypes provided herein (e.g. in Sections 2, 5.1, and 7), wherein the PRI above a cutoff is predictive of (i) an increase in a level of TNFSF15 (TL1A) protein expression in a sample obtained from a subject or patient, as compared to a reference level of TNFSF15 (TL1A) protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. In some embodiments of the methods provided herein (e.g. in this paragraph), the step of determining PRI, RPS, or MRS above the cutoff is performed using a system selected from the systems provided in Section 5.8. In some embodiments of the methods provided herein (e.g. in this paragraph), the step of determining PRI, RPS, or MRS above the cutoff is performed using a computer implemented method selected from the computer implemented methods provided in Section 5.8. In some embodiments of the methods provided herein (e.g. in this paragraph), the step of determining PRI, RPS, or MRS above the cutoff is performed as described in Sections 5.8 and 7.

A few methods were adopted and compared to determine the cutoff value for the MRS that was used for selecting patients responsive to a treatment with an inhibitor of TL1A expression or activity (e.g. TL1A CDx positive or TL1A CDx negative). Two of these methods give equal weight to sensitivity and specificity with no ethical, cost and prevalence constraints. The first method uses the square of distance between the point (0, 1) on the upper left hand corner of ROC space and any point on ROC curve, i.e. d²=(1-sensitivity)²+(1-specificity)². In order to obtain the optimal cut off points, the square of this distance is minimized. In other words, this distance for each point on the ROC curve from the point (0, 1) can be calculated to find the optimal cut-off value. The second method called Youden index uses the maximum of vertical distance of ROC curve from the point (x, y) on diagonal line (chance line). Youden index maximizes the difference between sensitivity and (1-specificity): Youden Index=Sensitivity+Specificity−1. Thus, by maximizing Sensitivity+Specificity across various points on the ROC curve, the optimal cut-off point is calculated. The third method incorporates the financial costs for correct and false diagnosis and the costs of further work up for diagnosis. In fact, the consequence of each possible test outcome is ascertained to their costs and combining ROC analysis with utility-based decision theory can be used to determine the optimal cutoff. For example, given a disease with low prevalence and high cost of false positive diagnosis, the cutoff may be chosen at higher value to maximize specificity while for a disease occurring at high prevalence and missing diagnosis has a serious fatal consequences, a lower cutoff value would be selected to maximize sensitivity. In a specific example, the cutoff value is set for a sensitivity of 100%, 95%, 90%, 85%, 80%, 75%, 70%, 65%, 60%, 55%, 50%, 45%, 40%, 35%, 30%, 25%, or 20%. In another example, the cutoff value can be set for a pre-determined positive predictive value, e.g. 95%, 90%, 85%, 80%, 75%, 70%, 65%, 60%, 55%, 51%, 50%, 45%, 40%, 35%, 34%, 33%, 32%, 31%, 30%, 29%, 28%, 27%, 26%, or 25%. In other examples, the cutoff value can be set for a pre-determined positive rate, e.g. 30-40%. In one example, the cutoff value is set for a positive rate of about 25%. In one example, the cutoff value is set for a positive rate of about 26%. In one example, the cutoff value is set for a positive rate of about 27%. In one example, the cutoff value is set for a positive rate of about 28%. In one example, the cutoff value is set for a positive rate of about 29%. In one example, the cutoff value is set for a positive rate of about 30%. In one example, the cutoff value is set for a positive rate of about 31%. In one example, the cutoff value is set for a positive rate of about 32%. In one example, the cutoff value is set for a positive rate of about 33%. In one example, the cutoff value is set for a positive rate of about 34%. In one example, the cutoff value is set for a positive rate of about 35%. In one example, the cutoff value is set for a positive rate of about 36%. In one example, the cutoff value is set for a positive rate of about 37%. In one example, the cutoff value is set for a positive rate of about 38%. In one example, the cutoff value is set for a positive rate of about 39%. In one example, the cutoff value is set for a positive rate of about 40%. In one example, the cutoff value is set for a positive rate of about 41%. In one example, the cutoff value is set for a positive rate of about 42%. In one example, the cutoff value is set for a positive rate of about 43%. In one example, the cutoff value is set for a positive rate of about 44%. In one example, the cutoff value is set for a positive rate of about 45%. In the fourth method, the MRS score is calculated as described above and the logistic function is then applied to the MRS to generate a continuous Response Probability Score (“RPS”), wherein in some examples RPS equals to 1/(1+e^{(-MRS)}) and ranges from 0 to 1. In some examples, the model is trained, optimized, and normalized (e.g. normalization of β coefficient) such that patients in the training cohort that responded to TL1A inhibitor therapy would have a RPS value≥0.5 and patients in the training cohort that did not respond to TL1A inhibitor therapy would have a RPS value<0.5. Accordingly, 0.5 can be the cutoff for the RPS for determining whether a patient would be a responder or non-responder to TL1A inhibitor therapy. If the RPS is ≥0.5, the prediction is “yes, responder”. If the RPS is <0.5, the prediction is “no, non-responder”.

As is clear from the previous paragraph, the cutoff can be determined to achieve any positive predictive value, specificity, sensitivity, negative predictive value, positive rate, or accuracy. Accordingly, in some embodiments of the various methods provided herein, including in Sections 2, 5 (including in the paragraphs of Section 5.2), and 7, the cutoff in the methods is such a cutoff that provide any one of the positive predictive values provided in Sections 2, 5 (including in the paragraphs of Section 5.2), and 7. In some embodiments of the various methods provided herein, including in Sections 2, 5 (including in the paragraphs of Section 5.2), and 7, the cutoff in the methods is such a cutoff that provide any one of the specificity provided in Sections 2, 5 (including in the paragraphs of Section 5.2), and 7. In some embodiments of the various methods provided herein, including in Sections 2, 5 (including in the paragraphs of Section 5.2), and 7, the cutoff in the methods is such a cutoff that provide any one of the sensitivity provided in Sections 2, 5 (including in the paragraphs of Section 5.2), and 7. In some embodiments of the various methods provided herein, including in Sections 2, 5 (including in the paragraphs of Section 5.2), and 7, the cutoff in the methods is such a cutoff that provide any one of the negative predictive values provided in Sections 2, 5 (including in the paragraphs of Section 5.2), and 7. In some embodiments of the various methods provided herein, including in Sections 2, 5 (including in the paragraphs of Section 5.2), and 7, the cutoff in the methods is such a cutoff that provide any one of the accuracy provided in Sections 2, 5 (including in the paragraphs of Section 5.2), and 7. In some embodiments of the various methods provided herein, including in Sections 2, 5 (including in the paragraphs of Section 5.2), and 7, the cutoff in the methods is such a cutoff that provide any one of the positive rates provided in Sections 2, 5 (including in the paragraphs of Section 5.2), and 7.

In one embodiment of the methods provided herein, including in Sections 2, 5 (including in the paragraphs of Section 5.2), and 7, the method further comprising determining a combination of polymorphisms provided herein, e.g. a combination of polymorphisms provided in Sections 2, 5.1, and 7. In one embodiment of the methods provided herein, including in Sections 2, 5 (including in the paragraphs of Section 5.2), and 7, the method further comprising determining a combination of genotypes provided herein, e.g. a combination of genotypes provided in Sections 2, 5.1, and 7. In some embodiments of the methods provided herein (e.g. in this paragraph), the step of determining the combination of genotypes or polymorphisms is performed as described in Sections 5.3, 5.9.5, and 7. In one embodiment of the methods provided herein, including in Sections 2, 5 (including in the paragraphs of Section 5.2), and 7, the method further comprises preparing a sample from the patient. In some embodiments of the methods provided herein (e.g. in this paragraph), the step of preparing a sample from the patient is performed as described in Section 5.9. In some embodiments of the methods provided herein (e.g. in this paragraph), the step of determining a combination of genotypes or polymorphisms comprises determining combination of genotypes or polymorphisms in the sample prepared as described in Sections 5.3, 5.9.5, and 7.

In one embodiment of the methods provided herein, including in Sections 2, 5 (including in the paragraphs of Section 5.2), and 7, the method further comprises preparing a sample from the patient and determining a combination of polymorphisms in the sample, e.g. a combination of polymorphisms provided in Sections 2, 5.1, and 7. In one embodiment of the methods provided herein, including in Sections 2, 5 (including in the paragraphs of Section 5.2), and 7, the method further comprises preparing a sample from the patient and determining a combination of genotypes in the sample, e.g. a combination of genotypes provided in Sections 2, 5.1, and 7. In some embodiments of the methods provided herein (e.g. in this paragraph), the step of preparing a sample from the patient is performed as described in Section 5.9. In some embodiments of the methods provided herein (e.g. in this paragraph), the step of determining the combination of genotypes or polymorphisms is performed as described in Sections 5.3, 5.9.5, and 7.

In one embodiment of the methods provided herein, including in Sections 2, 5 (including in the paragraphs of Section 5.2), and 7, the method further comprising preparing a sample from the patient, determining a combination of polymorphisms in the sample, e.g. a combination of polymorphisms provided in Sections 2, 5.1, and 7, and determining PRI above a cutoff, wherein the PRI above the cutoff is indicative (i) an increase in a level of TL1A protein expression in a sample obtained from a subject or patient, as compared to a reference level of TL1A protein expression in a tissue not affected by IBD, (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. In one embodiment of the methods provided herein, including in Sections 2, 5 (including in the paragraphs of Section 5.2), and 7, the method further comprising preparing a sample from the patient, determining a combination of genotypes in the sample, e.g. a combination of genotypes provided in Sections 2, 5.1, and 7, and determining PRI above a cutoff, wherein the PRI above the cutoff is indicative (i) an increase in a level of TL1A protein expression in a sample obtained from a subject or patient, as compared to a reference level of TL1A protein expression in a tissue not affected by IBD, (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. In some embodiments of the methods provided herein (e.g. in this paragraph), the step of preparing a sample from the patient is performed as described in Section 5.9. In some embodiments of the methods provided herein (e.g. in this paragraph), the step of determining the combination of genotypes or polymorphisms is performed as described in Sections 5.3, 5.9.5, and 7.

In some embodiments of the methods provided herein, the step of determining a positive result is performed using a system selected from the systems provided in Section 5.8. In some embodiments of the methods provided herein (e.g. in this paragraph), the step of determining a positive result is performed using a computer implemented method selected from the computer implemented methods provided in Section 5.8. In some embodiments of the methods provided herein (e.g. in this paragraph), the step of determining a PRI above a cutoff (or PRI below a cutoff when PRI negatively correlates with MRS or RPS) is performed as described in Sections 2, 5.2, and 7. In some embodiments of the methods provided herein (e.g. in this paragraph), the PRI, RPS or MRS is calculated as described in Sections 2, 5.2, and 7. In some embodiments of the methods provided herein (e.g. in this paragraph), the combination of polymorphisms comprises at least 2, 3, 4, 5, 6, 7, 8, 9, or 10 polymorphisms. In some embodiments of the methods provided herein (e.g. in this paragraph), the combination of polymorphisms comprises at least 2 polymorphisms. In some embodiments of the methods provided herein (e.g. in this paragraph), the combination of genotypes comprises at least 2, 3, 4, 5, 6, 7, 8, 9, or 10 genotypes. In some embodiments of the methods provided herein (e.g. in this paragraph), the combination of genotypes comprises at least 2 genotypes.

In one embodiment of the methods provided herein, including in Sections 2, 5 (including in the paragraphs of Section 5.2), and 7, the test comprises matching the combination of polymorphisms of the subject to a profile of polymorphisms set for positive results. In one embodiment of the methods provided herein, including in Sections 2, 5 (including in the paragraphs of Section 5.2), and 7, the test comprises matching the combination of polymorphisms of the subject to a profile of polymorphisms set for positive results, wherein the test is positive if there is a match with the profile of polymorphisms set for positive results.

In some embodiments of the methods provided herein, including in Sections 2, 5 (including in the paragraphs of Section 5.2), and 7, determining a positive result comprises (1) determining a positive result when the PRI is above a cutoff if the PRI has a positive correlation with Response Probability Score (RPS); or (2) determining a positive result when the PRI is below a cutoff if the PRI has a negative correlation with RPS, wherein the PRI is calculated from a combination of genotypes determined from a sample from the subject. In some embodiments of the methods provided herein, including in Sections 2, 5 (including in the paragraphs of Section 5.2), and 7, determining a positive result comprises (1) determining a positive result when the PRI is above a cutoff if the PRI has a positive correlation with Model Risk Score (MRS); or (2) determining a positive result when the PRI is below a cutoff if the PRI has a negative correlation with MRS, wherein the PRI is calculated from a combination of genotypes determined from a sample from the subject. In some embodiments of the methods provided herein, including in Sections 2, 5 (including in the paragraphs of Section 5.2), and 7, determining a positive result comprises determining a positive result when the RPS is above a cutoff, wherein the RPS is calculated from a combination of genotypes determined from a sample from the subject. In some embodiments of the methods provided herein, including in Sections 2, 5 (including in the paragraphs of Section 5.2), and 7, determining a positive result comprises determining a positive result when the MRS is above a cutoff, wherein the MRS is calculated from a combination of genotypes determined from a sample from the subject.

In one embodiment of the methods provided herein, including in Sections 2, 5 (including in the paragraphs of Section 5.2), and 7, having a positive correlation coefficient is having a positive Pearson correlation coefficient. In one embodiment of the methods provided herein, including in Sections 2, 5 (including in the paragraphs of Section 5.2), and 7, having a positive correlation coefficient is having a positive Spearman correlation coefficient. Additionally, for every embodiment of the methods provided herein, including in Sections 2, 5 (including in the paragraphs of Section 5.2), and 7, that recite “have [has, or having] a positive correlation coefficient,” the disclosure also provides a corresponding embodiment wherein the recitation of “have [has, or having] a positive correlation coefficient with” is substituted with “positively correlate with,” all else remaining identical. In one embodiment of the methods provided herein, including in Sections 2, 5 (including in the paragraphs of Section 5.2), and 7, having a negative correlation coefficient is having a negative Pearson correlation coefficient. In one embodiment of the methods provided herein, including in Sections 2, 5 (including in the paragraphs of Section 5.2), and 7, having a negative correlation coefficient is having a negative Spearman correlation coefficient. Additionally, for every embodiment of the methods provided herein, including in Sections 2, 5 (including in the paragraphs of Section 5.2), and 7, that recite “have [has, or having] a negative correlation coefficient,” the disclosure also provides a corresponding embodiment wherein the recitation of “have [has, or having] a negative correlation coefficient with” is substituted with “negatively correlate with,” all else remaining identical. In one embodiment of the methods provided herein, including in Sections 2, 5 (including in the paragraphs of Section 5.2), and 7, the correlation is determined by Pearson correlation coefficient. In one embodiment of the methods provided herein, including in Sections 2, 5 (including in the paragraphs of Section 5.2), and 7, the correlation is determined by Spearman correlation coefficient. In one embodiment of the methods provided herein, including in Sections 2, 5 (including in the paragraphs of Section 5.2), and 7, the correlation coefficient is Pearson correlation coefficient. In one embodiment of the methods provided herein, including in Sections 2, 5 (including in the paragraphs of Section 5.2), and 7, the correlation coefficient is Spearman correlation coefficient.

In one embodiment of the methods provided herein, including in Sections 2, 5 (including in the paragraphs of Section 5.2), and 7, the cutoff value in the method is such a value that maximizes the accuracy of predicting the therapeutic response to TL1A inhibitor treatment in a training subject cohort. In one embodiment of the methods provided herein, including in Sections 2, 5 (including in the paragraphs of Section 5.2), and 7, the cutoff value in the method is such a value that maximizes the PPV of predicting the therapeutic response to TL1A inhibitor treatment in a training subject cohort. In one embodiment of the methods provided herein, including in Sections 2, 5 (including in the paragraphs of Section 5.2), and 7, the cutoff value in the method is such a value that maximizes the NPV of predicting the therapeutic response to TL1A inhibitor treatment in a training subject cohort. In one embodiment of the methods provided herein, including in Sections 2, 5 (including in the paragraphs of Section 5.2), and 7, the cutoff value in the method is such a value that maximizes the sum of PPV and NPV of predicting the therapeutic response to TL1A inhibitor treatment in a training subject cohort. In one embodiment of the methods provided herein, including in Sections 2, 5 (including in the paragraphs of Section 5.2), and 7, the cutoff value in the method is such a value that maximizes the sum of specificity and sensitivity of predicting the therapeutic response to TL1A inhibitor treatment in a training subject cohort.

As described above, when the PRI has a negative correlation coefficient with RPS, e.g. PRI=−1×RPS, the PRI inversely correlate with (i) an increase in a level of TL1A protein expression in a sample obtained from a subject or patient, as compared to a reference level of TL1A protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. Therefore, when the PRI has a negative correlation coefficient with RPS, e.g. PRI=−1×RPS, the subject selected for the methods provided herein (including in Sections 2, 5.2, and 7) will be based on PRI below the cutoff, all else in the methods remaining identical. Accordingly, for every embodiment of method described herein (including Sections 2, 5.2, and 7) that provides PRI above a cutoff, the disclosure provides a corresponding embodiment in which the element of PRI above a cutoff is substituted by PRI below a cutoff wherein the PRI has a negative correlation coefficient with RPS, while keeping all else in the methods identical. Additionally, for every embodiment of method described herein (including Sections 2, 5.2, and 7) that provides PRI above a cutoff wherein the PRI has a positive correlation coefficient with RPS, the disclosure provides a corresponding embodiment in which the element of PRI above a cutoff wherein the PRI has a positive correlation coefficient with RPS is substituted by PRI below a cutoff wherein the PRI has a negative correlation coefficient with RPS, while keeping all else in the methods identical.

Similarly, when the PRI has a negative correlation coefficient with MRS, e.g. PRI=−1×MRS, the PRI inversely correlate with (i) an increase in a level of TL1A protein expression in a sample obtained from a subject or patient, as compared to a reference level of TL1A protein expression (e.g., derived from a normal individual), (ii) an increase of IBD enriched cell types in an IBD-affected tissue as compared to a reference level in a tissue not affected by IBD, (iii) a decrease of IBD depleted cell types in an IBD affected tissue as compared to a reference level in a tissue not affected by IBD, and/or (iv) an increase of a positive therapeutic response in IBD patients to a treatment with the TL1A inhibitor as compared to the reference level of response in patients not selected by the genotypes or models. Therefore, when the PRI has a negative correlation coefficient with MRS, e.g. PRI=−1×MRS, the subject selected for the methods provided herein (including in Sections 2, 5.2, and 7) will be based on PRI below the cutoff, all else in the methods remaining identical. Accordingly, for every embodiment of method described herein (including Sections 2, 5.2, and 7) that provides PRI above a cutoff, the disclosure provides a corresponding embodiment in which the element of PRI above a cutoff is substituted by PRI below a cutoff wherein the PRI has a negative correlation coefficient with MRS, while keeping all else in the methods identical. Additionally, for every embodiment of method described herein (including Sections 2, 5.2, and 7) that provides PRI above a cutoff wherein the PRI has a positive correlation coefficient with MRS, the disclosure provides a corresponding embodiment in which the element of PRI above a cutoff wherein the PRI has a positive correlation coefficient with MRS is substituted by PRI below a cutoff wherein the PRI has a negative correlation coefficient with MRS, while keeping all else in the methods identical.

In various embodiments of the methods or the computer-implemented systems provided herein, such as those provided in Sections 2, 5 (including in the paragraphs of Section 5.2 and paragraphs of Section 5.8), and 7, the subject has been treated with an advanced IBD therapy prior to the treatment with the inhibitor of TL1A activity or expression. In some embodiments of the methods or the computer-implemented systems provided herein, including in this paragraph, the subject has been treated with 1, 2, or 3 advanced IBD therapies prior to the treatment with the inhibitor of TL1A activity or expression. In some embodiments of the methods or the computer-implemented systems provided herein, the subject has not been treated with an advanced IBD therapy prior to the treatment with the inhibitor of TL1A activity or expression. In some embodiments of the methods or the computer-implemented systems provided herein, including in this paragraph, the advanced IBD therapy comprises one or more selected from the group consisting of a biologic therapeutic agent for IBD, an S1P1 modulator, or a JAK inhibitor. In some embodiments of the methods or the computer-implemented systems provided herein, including in this paragraph, the biologic therapeutic agent for IBD comprises an anti-TNFα antibody, an anti-IL23 antibody, or an anti-integrin antibody. In some embodiments of the methods or the computer-implemented systems provided herein, including in this paragraph, the anti-TNFα comprises adalimumab, infliximab, golimumab, certolizumab, or etanercept. In some embodiments of the methods or the computer-implemented systems provided herein, including in this paragraph, the anti-IL23 antibody comprises ustekinumab, guselkumab, risankizumab, brazikumab, mirikizumab, tildrakizumab, or briakinumab. In some embodiments of the methods or the computer-implemented systems provided herein, including in this paragraph, the anti-integrin antibody comprises etrolizumab, vedolizumab, natalizumab, or ontamalimab. In some embodiments of the methods or the computer-implemented systems provided herein, including in this paragraph, the S1P1 modulator comprises fingolimod, siponimod, etrasimod, ozanimod, ponesimod, amiselimod, ceralifimod, or mocravimod. In some embodiments of the methods or the computer-implemented systems provided herein, including in this paragraph, the JAK inhibitor comprises tofacitinib, abrocitinib, baricitinib, upadacitinib, or filgotinib.

In various embodiments, provided herein is a method of treating inflammatory bowel disease (IBD) in a subject in need thereof, comprising: administering to the subject a therapeutically effective amount of an antibody or an antigen-binding fragment that specifically binds TL1A. In some embodiments of the methods provided herein, such as those provided in Sections 2, 5 (including in the paragraphs of Section 5.2), and 7, the subjects can be selected by various selection criteria described in Sections 2, 5 (including in the paragraphs of Sections 5.1 and 5.2), and 7. In some embodiments of the methods provided herein, such as those provided in Sections 2, 5 (including in the paragraphs of Section 5.2), and 7, the subjects can be selected by various selection criteria described in Sections 2, 5 (including in the paragraphs of Sections 5.1 and 5.2), and 7, and the inhibitor of TL1A activity or expression can be any one described in Section 2, 5.4 and 7. In some embodiments of the methods provided herein, such as those provided in Sections 2, 5 (including in the paragraphs of Section 5.2), and 7, the inhibitor of TL1A activity or expression can be any one described in Section 2, 5.4 and 7. In some embodiments, the anti-TL1A antibody comprises antibody A. In some embodiments, the anti-TL1A antibody comprises antibody B. In some embodiments, the anti-TL1A antibody comprises antibody C. In some embodiments, the anti-TL1A antibody comprises antibody D. In some embodiments, the anti-TL1A antibody comprises antibody E. In some embodiments, the anti-TL1A antibody comprises antibody F. In some embodiments, the anti-TL1A antibody comprises antibody G. In some embodiments, the anti-TL1A antibody comprises antibody I. In some embodiments, the anti-TL1A antibody comprises antibody H. In some embodiments, the anti-TL1A antibody comprises antibody A2. In some embodiments, the anti-TL1A antibody comprises antibody B2. In some embodiments, the anti-TL1A antibody comprises antibody C2. In some embodiments, the anti-TL1A antibody comprises antibody D2. In some embodiments, the anti-TL1A antibody comprises antibody E2. In some embodiments, the anti-TL1A antibody comprises antibody F2. In some embodiments, the anti-TL1A antibody comprises antibody G2. In some embodiments, the anti-TL1A antibody comprises antibody I2. In some embodiments, the anti-TL1A antibody comprises antibody H2. In certain embodiments, the anti-TL1A antibody comprises any one of the antibodies of Table 10. In some embodiments, the anti-TL1A antibody comprises antibody A217. In some embodiments, the anti-TL1A antibody comprises antibody A220. In some embodiments, the anti-TL1A antibody comprises antibody A223. In some embodiments, the anti-TL1A antibody comprises antibody A219. In some embodiments, the anti-TL1A antibody comprises antibody A221. In some embodiments, the anti-TL1A antibody comprises antibody A200. In some embodiments, the anti-TL1A antibody comprises antibody A213. In some embodiments, the anti-TL1A antibody comprises antibody A212. In some embodiments, the anti-TL1A antibody comprises antibody A107. In some embodiments, the anti-TL1A antibody comprises antibody A205. In some embodiments, the anti-TL1A antibody comprises antibody A211. In some embodiments, the anti-TL1A antibody comprises antibody A199. In some embodiments, the anti-TL1A antibody comprises antibody A15. In some embodiments, the anti-TL1A antibody comprises antibody A30. In some embodiments, the anti-TL1A antibody comprises antibody A100. In some embodiments, the anti-TL1A antibody comprises antibody A181. In some embodiments, the anti-TL1A antibody comprises antibody A129. In some embodiments, the anti-TL1A antibody comprises antibody A214. In some embodiments, the anti-TL1A antibody comprises antibody A216. In some embodiments, the anti-TL1A antibody comprises antibody A122. In some embodiments, the anti-TL1A antibody comprises antibody A222. In some embodiments, the anti-TL1A antibody comprises antibody A188. In some embodiments, the anti-TL1A antibody comprises antibody A203. In some embodiments, the anti-TL1A antibody comprises antibody A147. In some embodiments, the anti-TL1A antibody comprises antibody A127. In some embodiments, the anti-TL1A antibody comprises antibody A126. In some embodiments, the anti-TL1A antibody comprises antibody A160. In some embodiments, the anti-TL1A antibody comprises antibody A157. In some embodiments, the anti-ILIA antibody comprises antibody A159. In some embodiments, the anti-TL1A antibody comprises antibody A218. In some embodiments, the anti-TL1A antibody comprises antibody A158. In some embodiments, the anti-TL1A antibody comprises antibody A125. In some embodiments, the anti-TL1A antibody comprises antibody A103. In some embodiments, the anti-TL1A antibody comprises antibody A64. In some embodiments, the anti-TL1A antibody comprises antibody A67. In some embodiments, the anti-TL1A antibody comprises antibody A138. In some embodiments, the anti-TL1A antibody comprises antibody A68. In some embodiments, the anti-TL1A antibody comprises antibody A94. In some embodiments, the anti-TL1A antibody comprises antibody A110. In some embodiments, the anti-TL1A antibody comprises antibody A197. In some embodiments, the anti-TL1A antibody comprises antibody A112. In some embodiments, the anti-TL1A antibody comprises antibody A169. In some embodiments, the anti-TL1A antibody comprises antibody A173. In some embodiments, the anti-TL1A antibody comprises antibody A179. In some embodiments, the anti-TL1A antibody comprises antibody A148. In some embodiments, the anti-TL1A antibody comprises antibody A115. In some embodiments, the anti-TL1A antibody comprises antibody A149. In some embodiments, the anti-TL1A antibody comprises antibody A134. In some embodiments, the anti-TL1A antibody comprises antibody A113. In some embodiments, the anti-TL1A antibody comprises antibody A151. In some embodiments, the anti-TL1A antibody comprises antibody A96. In some embodiments, the anti-TL1A antibody comprises antibody A132. In some embodiments, the anti-TL1A antibody comprises antibody A196. In some embodiments, the anti-TL1A antibody comprises antibody A172. In some embodiments, the anti-TL1A antibody comprises antibody A75. In some embodiments, the anti-TL1A antibody comprises antibody A174. In some embodiments, the anti-TL1A antibody comprises antibody A109. In some embodiments, the anti-TL1A antibody comprises antibody A198. In some embodiments, the anti-TL1A antibody comprises antibody A170. In certain embodiments, the anti-TL1A antibody comprises any one of the antibodies of Tables 20-21. In some embodiments, the anti-TL1A antibody comprises antibody clone 34. In some embodiments, the anti-TL1A antibody comprises antibody 5C3D11. In some embodiments, the anti-TL1A antibody comprises antibody 9E12E5. In some embodiments, the anti-TL1A antibody comprises antibody AS12824. In some embodiments, the anti-TL1A antibody comprises antibody AS12823. In some embodiments, the anti-TL1A antibody comprises antibody AS12819. In some embodiments, the anti-TL1A antibody comprises antibody AS12816. In some embodiments, the anti-TL1A antibody comprises antibody AS12825. In some embodiments, the anti-TL1A antibody comprises antibody 12835. In some embodiments, the anti-TL1A antibody comprises antibody 18-7 S93E. In some embodiments, the anti-TL1A antibody comprises antibody 18-7. In some embodiments, the anti-TL1A antibody comprises antibody 18-7 S92D. In some embodiments, the anti-TL1A antibody comprises antibody 18-7 S92H. In some embodiments, the anti-TL1A antibody comprises antibody 18-7 S92N. In some embodiments, the anti-TL1A antibody comprises antibody 18-7 S92Q. In some embodiments, the anti-TL1A antibody comprises antibody 18-7 CDRv. In some embodiments, the anti-TL1A antibody comprises antibody 21-3. In some embodiments, the anti-TL1A antibody comprises antibody 21-3 V102K. In some embodiments, the anti-TL1A antibody comprises antibody 21-3 V102M. In some embodiments, the anti-TL1A antibody comprises antibody 21-3 V102Q. In some embodiments, the anti-TL1A antibody comprises antibody 21-3 V102 W. In some embodiments, the anti-TL1A antibody comprises antibody 21-3 CDRv. In some embodiments, the anti-TL1A antibody comprises antibody 21-3 CDRv. In some embodiments, the anti-TL1A antibody comprises antibody clone 2. In some embodiments, the anti-TL1A antibody comprises antibody clone 52. In some embodiments, the anti-TL1A antibody comprises antibody clone 46. In some embodiments, the anti-TL1A antibody comprises antibody clone 47. In some embodiments, the anti-TL1A antibody comprises antibody clone 14. In some embodiments, the anti-TL1A antibody comprises antibody clone 16L. In some embodiments, the anti-TL1A antibody comprises antibody clone 17L. In some embodiments, the anti-TL1A antibody comprises antibody clone 17L-1. In some embodiments, the anti-TL1A antibody comprises antibody clone 23. In some embodiments, the anti-TL1A antibody comprises antibody clone 53. In some embodiments, the anti-TL1A antibody comprises antibody clone E1. In some embodiments, the anti-TL1A antibody comprises antibody clone 3-17L V-A. In some embodiments, the anti-TL1A antibody comprises antibody clone 3-17L. In some embodiments, the anti-TL1A antibody comprises antibody clone L8 mod. In some embodiments, the anti-TL1A antibody comprises antibody clone X-V. In some embodiments, the anti-TL1A antibody comprises antibody clone X. In some embodiments, the anti-TL1A antibody comprises antibody clone XL3-6. In some embodiments, the anti-TL1A antibody comprises antibody clone XL3-10. In some embodiments, the anti-TL1A antibody comprises antibody clone XL3-15. In some embodiments, the anti-TL1A antibody comprises antibody clone L3-13. In some embodiments, the anti-TL1A antibody comprises antibody clone H3-1. In some embodiments, the anti-TL1A antibody comprises antibody clone H2-2. In some embodiments, the anti-TL1A antibody comprises antibody clone H2-5. In some embodiments, the anti-TL1A antibody comprises antibody M1. In some embodiments, the anti-TL1A antibody comprises antibody M2. In some embodiments, the anti-TL1A antibody comprises antibody M3. In some embodiments, the anti-TL1A antibody comprises antibody M4. In some embodiments, the anti-TL1A antibody comprises antibody M5. In some embodiments, the anti-TL1A antibody comprises antibody M6. In some embodiments, the anti-TL1A antibody comprises antibody M7. In some embodiments, the anti-TL1A antibody comprises antibody M8. In some embodiments, the anti-TL1A antibody comprises antibody M9. In some embodiments, the anti-TL1A antibody comprises antibody M10. In some embodiments, the anti-TL1A antibody comprises antibody M11. In some embodiments, the anti-TL1A antibody comprises antibody M12.

Methods disclosed herein provide methods of treating an inflammatory bowel disease (IBD) in a subject by administering an anti-TL1A antibody described herein to the subject. In various embodiments, IBD is Crohn's Disease (CD) or ulcerative colitis (UC). In some embodiments, the IBD is a severe form of IBD. In some embodiments, the IBD is a moderate to severe form of IBD. In some embodiments, the IBD is a moderate form of IBD. In various other embodiments, the subject is determined to have an increased TL1A expression. In some embodiments, the administration of a therapeutically effective amount of an anti-TL1A antibody causes a decrease in TL1A in the subject treated.

### 5.3 Methods of Detection

Methods disclosed herein for detecting a genotype in a sample from a subject comprise analyzing the genetic material in the sample to detect at least one of a presence, an absence, and a quantity of a nucleic acid sequence encompassing the genotype of interest and administering an anti-TL1A antibody or antigen binding fragment as disclosed herein. In some embodiments, the sample is assayed to measure a presence, absence or quantity of at least three polymorphisms. In some embodiments, the sample is assayed to measure a presence, absence, or quantity of at least four polymorphisms. In some embodiments, the sample is assayed to measure a presence, absence, or quantity of at least five polymorphisms. In some embodiments, the sample is assayed to measure a presence, absence, or quantity of at least six polymorphisms. In some embodiments, the sample is assayed to measure a presence, absence, or quantity of at least seven polymorphisms. In some embodiments, the sample is assayed to measure a presence, absence, or quantity of at least eight polymorphisms. In some embodiments, at least three genotypes are detected, using the methods described herein. In some embodiments, at least eight genotypes are detected, using the methods described herein.

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In one embodiment, determining the genotype of a gene is performed at the nucleic acid level by performing RNA-seq, a reverse transcriptase polymerase chain reaction (RT-PCR) or a hybridization assay with oligonucleotides that are substantially complementary to portions of cDNA molecules of the at least one biomarker gene under conditions suitable for RNA-seq, RT-PCR or hybridization and obtaining expression levels of the at least one biomarker gene.

In another embodiment, determining the genotype of a gene is performed at the nucleic acid level by performing DNA sequencing as described herein, a polymerase chain reaction (PCR, e.g., real time PCR or quantitative PCR) and/or a hybridization assay with oligonucleotides that are substantially complementary to portions of amplified DNA molecules of the gene under conditions suitable for hybridization, thereby obtaining the genotype of the biomarker genes.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Exemplary probes comprise a nucleic acid sequence of at least 10 contiguous nucleic acids provided in any one of SEQ ID NOS: 2001-2048, or 2057-2059, including the nucleobase indicated with a non-nucleobase letter (e.g., R, N, S), or a reverse complement thereof. In some instances, the probes may be used to detect the polymorphisms provided in Table 1, wherein the probe comprises a nucleic acid sequence of at least 10 contiguous nucleic acids provided in a corresponding SEQ ID NO or reverse complement thereof, the 10 contiguous nucleic acids comprising the “risk allele” also provided in Table 1 at a nucleoposition indicated with the non-nucleobase letter, or reverse complement thereof. In some embodiments, the probe comprises at least 70%, 80%, 85%, 90%, 91%, 92%, 93%, 94%, 95%, 96%, 97%, 98% or 99% sequence identity to any one of SEQ ID NOS: 2001-2048, or 2057-2059, or its reverse complement. In some instances, forward and reverse primers are used to amplify the target nucleic acid sequence. Forward and reverse primers may comprise a nucleic acid sequence flanking the risk allele provided in Table 1 corresponding to the nucleic acid sequence provided in any one of SEQ ID NOS: 2001-2048, or 2057-2059, or a reverse complement thereof.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. PCT primers and probes can be designed with tools known and used in the art. For example, forward and reverse primers for regions containing SNPs can be designed by uploading the flanking sequences into the Thermofisher OligoPerfect Primer Designer tool. The primer set with longest amplicon can be selected for the forward and reverse primers. Flanking sequences of SNPs can be obtained from the NCBI dbSNP database. Probes can be designed with the Thermofisher SNP genotype tool. The resulting probe design from the SNP genotype tool can be then truncated to 10-20 nucleotide flanks for the final design. Non-limiting examples of primer pairs useful for detecting one or more polymorphisms described herein are provided in Table 2, below.

TABLE 2
Exemplary Primer Sequences

| rsID | Forward Primer | Reverse Primer | Wt_Probe_Hex | Mut_Probe_FAM |
|---|---|---|---|---|
| rs6478109 | TGCTTCTGGAAGT | TGAGGTTCAAAA | TG+C +AG+A | TG+C +AGG |
| | GAAAGT (SEQ ID | TGACAGAGG | +T+TG GGA | +TTG GGA (SEQ |
| | NO: 2301) | (SEQ ID NO: 2311) | (SEQ ID NO: | ID NO: 2331) |
| | | | 2321) | |
| rs1892231 | GTCATCATCGCTT | TTT TCA ATG | AT +T+TG | ATT TGG |
| | TCATGTG (SEQ ID | CAC AGA TTT | +G+A+A | +C+A+A GGG |
| | NO: 2302) | AAG GA (SEQ ID | AGGG+AA (SEQ | AA (SEQ ID NO: |
| | | NO: 2312) | ID NO: 2322) | 2332) |
| rs7935393 | CTGGATGCTCAC | CCT AAG GAG | AG+A A+T+A | T+AG +AA+T |
| | AGGTTTG (SEQ ID | ACT TTT AGT TCT | +CA+C A+AG | +C+C+A CAA |
| | NO: 2303) | AAG (SEQ ID NO: | GA (SEQ ID NO: | (SEQ ID NO: |
| | | 2313) | 2323) | 2333) |
| rs7278257 | AGTCCCTGTTCTG | ATGGGGAACGTT | TC+C +TA+G | T+C+C TA+G |
| | AATCCTCT (SEQ | GTGGCAG (SEQ | +C+G+A TA | +G+GA TA (SEQ |
| | ID NO: 2304) | ID NO: 2314) | (SEQ ID NO: | ID NO: 2334) |
| | | | 2324) | |
| rs2070557 | CTT TTT GTC TCC | CGG CAG CCA | CGG GC+A | TCG GGC |
| | TAC CTC AGA GG | GAC AGG TAA | +C+AG C+TC | +TC+A GC+T C |
| | (SEQ ID NO: 2305) | (SEQ ID NO: 2315) | (SEQ ID NO: | (SEQ ID NO: |
| | | | 2325) | 2335) |
| rs9806914 | ATAAGAACCTCT | ACAGAGGCAGTA | A+GT +GAT | AGT GAT |
| | GCTGCACA (SEQ | TAGCACAG (SEQ | T+G+A +CT+C | T+G+G +CTC AA |
| | ID NO: 2306) | ID NO: 2316) | AA (SEQ ID NO: | (SEQ ID NO: |
| | | | 2326) | 2336) |
| rs16901748 | TTGGGAATCAGA | ATC AAG TCA | C+CA TTA | A+C+C ATT |
| | TAGGTGCA (SEQ | CAA CTG CCA GA | A+A+G +T+CA | AA+A +T+T+C |
| | ID NO: 2307) | (SEQ ID NO: 2317) | +GA (SEQ ID | AGA (SEQ ID |
| | | | NO: 2327) | NO: 2337) |
| rs56124762 | AAA CAG GAA | GCTCTGCCTTCA | TAG +T+T+A | TAG T+T+A |
| | CAG GCT GGT TC | CATTTCTG (SEQ | +A+G+C CCAT | +G+GC CCA |
| | (SEQ ID NO: 2308) | ID NO: 2318) | (SEQ ID NO: | (SEQ ID NO: |
| | | | 2328) | 2338) |
| rs2070558 | CCAAGCCAGTCC | AAT GAC CAG | AGG GAC | AGG GAC |
| | CAGTAG (SEQ ID | ATC CAA ATG | +C+G+C TGA | +C+A+C +TGA |
| | NO: 2309) | AGG (SEQ ID NO: | (SEQ ID NO: | (SEQ ID NO: |
| | | 2319) | 2329) | 2339) |
| rs2070561 | TTG GCA AGG | GTC CCC TGG | CGG T+G+C | CGG TGC +C+TC |
| | TTT CAG GTT TG | TCT CCC TGT C | +T+TC GTC C | GTC C (SEQ ID |
| | (SEQ ID NO: 2310) | (SEQ ID NO: 2320) | (SEQ ID NO: | NO: 2340) |
| | | | 2330) | |
| rs56124762 | AAACAGGAACAG | GCTCTGCCTTCA | +T+T+A | TAG T+T+A |
| | GCTGGTTC (SEQ | CATTTCTG (SEQ | +A+G+CCCAT' | +G+GC CCA |
| | ID NO: 2341) | ID NO: 2345) | (SEQ ID NO: | (SEQ ID NO: |
| | | | 2349) | 2353) |
| rs16901748 | GATCTTGGGAAT | ACAACTGCCAGA | CAT +TAA | CCA +T+TA |
| | CAGATAGGT (SEQ | CATATTTG (SEQ | +A+G+T+CAG | AA+T |
| | ID NO: 2342) | ID NO: 2346) | A+GT (SEQ ID | T+CA+GA+GT |
| | | | NO: 2350) | (SEQ ID NO: |
| | | | | 2354) |
| rs12934476 | GAAATCAGGTTA | CCCACCAGCCCA | CAT +TAA | AT+TTAA+T+G+ |
| | GAAATACACACA | TGTTATT (SEQ ID | +A+G+T+CAG | CTAA+C+GT |
| | TTA (SEQ ID NO: | NO: 2347) | A+GT (SEQ ID | (SEQ ID NO: |
| | 2343) | | NO: 2351) | 2355) |
| rs2297437 | CACCCCATTTCTG | AGGATGCCGATT | ATT C+G+G | ATT C+A+G |
| | CTTTCTG (SEQ ID | CTTCACA (SEQ ID | GT+G TGCTTG | GT+G TG+CTTG |
| | NO: 2344) | NO: 2348) | (SEQ ID NO: | (SEQ ID NO: |
| | | | 2352) | 2356) |

“Wt_Probe_Hex” and “Mut_Probe_FAM” mean “Wild type_probes_tagged with HEX reporter dye” and “Mut_probe_tagged with FAM reporter dye”, respectively. “+” stands for LNA bases (Locked nucleotides), which are analogues that are modified at 2′-O, 4′-C and form a bridge. This bridge results in restricted base pairing giving room to adjust the Tm as needed between the probes. Thus, +A, +T, +C or +G signify A, T, G or C bases are added on the modified backbone.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the target nucleic acid is at least 10 contiguous nucleic acid molecules of SEQ ID NO: 2001 comprising a non-reference allele at nucleoposition 501 within SEQ ID NO: 2001. In some embodiments, the target nucleic acid is at least 10 contiguous nucleic acid molecules of SEQ ID NO: 2001 comprising a “G” or an “A” allele at nucleoposition 501 within SEQ ID NO: 2001. In some embodiments, detecting the at least 10 contiguous nucleic acid molecules comprising a “G” or an “A” allele at nucleoposition 501 within SEQ ID NO: 2001 is sufficient to detect the polymorphism at rs11897732.

In some embodiments, the target nucleic acid is at least 10 contiguous nucleic acid molecules of SEQ ID NO: 2002 comprising non-reference allele at nucleoposition 501 within SEQ ID NO: 2002. In some embodiments, the target nucleic acid is at least 10 contiguous nucleic acid molecules of SEQ ID NO: 2002 comprising an “A” or a “G” allele at nucleoposition 501 within SEQ ID NO: 2002. In some embodiments, detecting the at least 10 contiguous nucleic acid molecules comprising an “A” or a “G” allele at nucleoposition 501 within SEQ ID NO: 2002 is sufficient to detect the polymorphism at rs6740739.

In some embodiments, the target nucleic acid is at least 10 contiguous nucleic acid molecules of SEQ ID NO: 2003 comprising a non-reference allele at nucleoposition 501 within SEQ ID NO: 2003. In some embodiments, the target nucleic acid is at least 10 contiguous nucleic acid molecules of SEQ ID NO: 2003 comprising a “G” or a “C” allele at nucleoposition 501 within SEQ ID NO: 2003. In some embodiments, detecting the at least 10 contiguous nucleic acid molecules comprising a “G” or a “C” allele at nucleoposition 501 within SEQ ID NO: 2003 is sufficient to detect the polymorphism at rs17796285.

In some embodiments, the target nucleic acid is at least 10 contiguous nucleic acid molecules of SEQ ID NO: 2004 comprising a non-reference allele at nucleoposition 501 within SEQ ID NO: 2004. In some embodiments, the target nucleic acid is at least 10 contiguous nucleic acid molecules of SEQ ID NO: 2004 comprising a “C” or an “A” allele at nucleoposition 501 within SEQ ID NO: 2004. In some embodiments, detecting the at least 10 contiguous nucleic acid molecules comprising a “C” or an “A” allele at nucleoposition 501 within SEQ ID NO: 2004 is sufficient to detect the polymorphism at rs7935393.

In some embodiments, the target nucleic acid is at least 10 contiguous nucleic acid molecules of SEQ ID NO: 2005 comprising a non-reference allele at nucleoposition 501 within SEQ ID NO: 2005. In some embodiments, the target nucleic acid is at least 10 contiguous nucleic acid molecules of SEQ ID NO: 2005 comprising a “G” or an “A” allele at nucleoposition 501 within SEQ ID NO: 2005. In some embodiments, detecting the at least 10 contiguous nucleic acid molecules comprising a “G” or an “A” allele at nucleoposition 501 within SEQ ID NO: 2005 is sufficient to detect the polymorphism at rs12934476.

In some embodiments, the target nucleic acid is at least 10 contiguous nucleic acid molecules of SEQ ID NO: 2006 comprising a non-reference allele at nucleoposition 501 within SEQ ID NO: 2006. In some embodiments, the target nucleic acid is at least 10 contiguous nucleic acid molecules of SEQ ID NO: 2006 comprising an “A” or a “C” allele at nucleoposition 501 within SEQ ID NO: 2006. In some embodiments, detecting the at least 10 contiguous nucleic acid molecules comprising an “A” or a “C” allele at nucleoposition 501 within SEQ ID NO: 2006 is sufficient to detect the polymorphism at rs12457255.

In some embodiments, the target nucleic acid is at least 10 contiguous nucleic acid molecules of SEQ ID NO: 2007 comprising a non-reference allele at nucleoposition 501 within SEQ ID NO: 2007. In some embodiments, the target nucleic acid is at least 10 contiguous nucleic acid molecules of SEQ ID NO: 2007 comprising an “A” or a “T” allele at nucleoposition 501 within SEQ ID NO: 2007. In some embodiments, detecting the at least 10 contiguous nucleic acid molecules comprising an “A” or a “T” allele at nucleoposition 501 within SEQ ID NO: 2007 is sufficient to detect the polymorphism at rs2070557.

In some embodiments, the target nucleic acid is at least 10 contiguous nucleic acid molecules of SEQ ID NO: 2008 comprising a non-reference allele at nucleoposition 501 within SEQ ID NO: 2008. In some embodiments, the target nucleic acid is at least 10 contiguous nucleic acid molecules of SEQ ID NO: 2008 comprising an “A” or a “G” allele at nucleoposition 501 within SEQ ID NO: 2008. In some embodiments, detecting the at least 10 contiguous nucleic acid molecules comprising an “A” or “G” allele at nucleoposition 501 within SEQ ID NO: 2008 is sufficient to detect the polymorphism at rs4246905.

In some embodiments, the target nucleic acid is at least 10 contiguous nucleic acid molecules of SEQ ID NO: 2009 comprising a non-reference allele at nucleoposition 501 within SEQ ID NO: 2009. In some embodiments, the target nucleic acid is at least 10 contiguous nucleic acid molecules of SEQ ID NO: 2009 comprising an “A” or a “G” allele at nucleoposition 501 within SEQ ID NO: 2009. In some embodiments, detecting the at least 10 contiguous nucleic acid molecules comprising an “A” or a “G” allele at nucleoposition 501 within SEQ ID NO: 2009 is sufficient to detect the polymorphism at rs10974900.

In some embodiments, the target nucleic acid is at least 10 contiguous nucleic acid molecules of SEQ ID NO: 2010 comprising a non-reference allele at nucleoposition 501 within SEQ ID NO: 2010. In some embodiments, the target nucleic acid is at least 10 contiguous nucleic acid molecules of SEQ ID NO: 2010 comprising a “C” or an “A” allele at nucleoposition 501 within SEQ ID NO: 2010. In some embodiments, detecting the at least 10 contiguous nucleic acid molecules comprising a “C” or an “A” allele at nucleoposition 501 within SEQ ID NO: 2010 is sufficient to detect the polymorphism at rs12434976.

In some embodiments, the target nucleic acid is at least 10 contiguous nucleic acid molecules of SEQ ID NO: 2011 comprising a non-reference allele at nucleoposition 501 within SEQ ID NO: 2011. In some embodiments, the target nucleic acid is at least 10 contiguous nucleic acid molecules of SEQ ID NO: 2011 comprising an “A” or a “C” allele at nucleoposition 501 within SEQ ID NO: 2011. In some embodiments, detecting the at least 10 contiguous nucleic acid molecules comprising an “T” or a “G” allele at nucleoposition 501 within SEQ ID NO: 2011 is sufficient to detect the polymorphism at rs16901748.

In some embodiments, the target nucleic acid is at least 10 contiguous nucleic acid molecules of SEQ ID NO: 2012 comprising a non-reference allele at nucleoposition 501 within SEQ ID NO: 2012. In some embodiments, the target nucleic acid is at least 10 contiguous nucleic acid molecules of SEQ ID NO: 2012 comprising an “A” or a “G” allele at nucleoposition 501 within SEQ ID NO: 2012. In some embodiments, detecting the at least 10 contiguous nucleic acid molecules comprising an “A” or a “G” allele at nucleoposition 501 within SEQ ID NO: 2012 is sufficient to detect the polymorphism at rs2815844.

In some embodiments, the target nucleic acid is at least 10 contiguous nucleic acid molecules of SEQ ID NO: 2013 comprising a non-reference allele at nucleoposition 501 within SEQ ID NO: 2013. In some embodiments, the target nucleic acid is at least 10 contiguous nucleic acid molecules of SEQ ID NO: 2013 comprising a “G” or an “A” allele at nucleoposition 501 within SEQ ID NO: 2013. In some embodiments, detecting the at least 10 contiguous nucleic acid molecules comprising a “G” or an “A” allele at nucleoposition 501 within SEQ ID NO: 2013 is sufficient to detect the polymorphism at rs889702.

In some embodiments, the target nucleic acid is at least 10 contiguous nucleic acid molecules of SEQ ID NO: 2014 comprising a non-reference allele at nucleoposition 501 within SEQ ID NO: 2014. In some embodiments, the target nucleic acid is at least 10 contiguous nucleic acid molecules of SEQ ID NO: 2014 comprising a “C” or an “A” allele at nucleoposition 501 within SEQ ID NO: 2014. In some embodiments, detecting the at least 10 contiguous nucleic acid molecules comprising a “C” or an “A” allele at nucleoposition 501 within SEQ ID NO: 2014 is sufficient to detect the polymorphism at rs2409750.

In some embodiments, the target nucleic acid is at least 10 contiguous nucleic acid molecules of SEQ ID NO: 2015 comprising a non-reference allele at nucleoposition 501 within SEQ ID NO: 2015. In some embodiments, the target nucleic acid is at least 10 contiguous nucleic acid molecules of SEQ ID NO: 2015 comprising an “A” or a “G” allele at nucleoposition 501 within SEQ ID NO: 2015. In some embodiments, detecting the at least 10 contiguous nucleic acid molecules comprising an “A” or “G” allele at nucleoposition 501 within SEQ ID NO: 2015 is sufficient to detect the polymorphism at rs1541020.

In some embodiments, the target nucleic acid is at least 10 contiguous nucleic acid molecules of SEQ ID NO: 2016 comprising a non-reference allele at nucleoposition 501 within SEQ ID NO: 2016. In some embodiments, the target nucleic acid is at least 10 contiguous nucleic acid molecules of SEQ ID NO: 2016 comprising a “T” or an “A” allele at nucleoposition 501 within SEQ ID NO: 2016. In some embodiments, detecting the at least 10 contiguous nucleic acid molecules comprising a “T” or an “A” allele at nucleoposition 501 within SEQ ID NO: 2016 is sufficient to detect the polymorphism at rs4942248.

In some embodiments, the target nucleic acid is at least 10 contiguous nucleic acid molecules of SEQ ID NO: 2017 comprising a non-reference allele at nucleoposition 501 within SEQ ID NO: 2017. In some embodiments, the target nucleic acid is at least 10 contiguous nucleic acid molecules of SEQ ID NO: 2017 comprising a “G” or an “A” allele at nucleoposition 501 within SEQ ID NO: 2017. In some embodiments, detecting the at least 10 contiguous nucleic acid molecules comprising a “G” or an “A” allele at nucleoposition 501 within SEQ ID NO: 2017 is sufficient to detect the polymorphism at rs12934476.

In some embodiments, the target nucleic acid is at least 10 contiguous nucleic acid molecules of SEQ ID NO: 2018 comprising a non-reference allele at nucleoposition 501 within SEQ ID NO: 2018. In some embodiments, the target nucleic acid is at least 10 contiguous nucleic acid molecules of SEQ ID NO: 2018 comprising an “A” or a “C” allele at nucleoposition 501 within SEQ ID NO: 2018. In some embodiments, detecting the at least 10 contiguous nucleic acid molecules comprising an “A” or a “C” allele at nucleoposition 501 within SEQ ID NO: 2018 is sufficient to detect the polymorphism at rs12457255.

In some embodiments, the target nucleic acid is at least 10 contiguous nucleic acid molecules of SEQ ID NO: 2019 comprising a non-reference allele at nucleoposition 501 within SEQ ID NO: 2019. In some embodiments, the target nucleic acid is at least 10 contiguous nucleic acid molecules of SEQ ID NO: 2019 comprising an “A” or a “G” allele at nucleoposition 501 within SEQ ID NO: 2019. In some embodiments, detecting the at least 10 contiguous nucleic acid molecules comprising an “A” or a “G” allele at nucleoposition 501 within SEQ ID NO: 2019 is sufficient to detect the polymorphism at rs2297437.

In some embodiments, the target nucleic acid is at least 10 contiguous nucleic acid molecules of SEQ ID NO: 2020 comprising a non-reference allele at nucleoposition 501 within SEQ ID NO: 2020. In some embodiments, the target nucleic acid is at least 10 contiguous nucleic acid molecules of SEQ ID NO: 2020 comprising a “G” or an “A” allele at nucleoposition 501 within SEQ ID NO: 2020. In some embodiments, detecting the at least 10 contiguous nucleic acid molecules comprising a “G” or an “A” allele at nucleoposition 501 within SEQ ID NO: 2020 is sufficient to detect the polymorphism at rs41309367.

In some embodiments, the target nucleic acid is at least 10 contiguous nucleic acid molecules of SEQ ID NO: 2021 comprising a non-reference allele at nucleoposition 501 within SEQ ID NO: 2021. In some embodiments, the target nucleic acid is at least 10 contiguous nucleic acid molecules of SEQ ID NO: 2021 comprising an “A” or a “G” allele at nucleoposition 501 within SEQ ID NO: 2021. In some embodiments, detecting the at least 10 contiguous nucleic acid molecules comprising an “A” or a “G” allele at nucleoposition 501 within SEQ ID NO: 2021 is sufficient to detect the polymorphism at rs10733509.

In some embodiments, the target nucleic acid is at least 10 contiguous nucleic acid molecules of SEQ ID NO: 2022 comprising a non-reference allele at nucleoposition 501 within SEQ ID NO: 2022. In some embodiments, the target nucleic acid is at least 10 contiguous nucleic acid molecules of SEQ ID NO: 2022 comprising a “G” or an “A” allele at nucleoposition 501 within SEQ ID NO: 2022. In some embodiments, detecting the at least 10 contiguous nucleic acid molecules comprising a “G” or an “A” allele at nucleoposition 501 within SEQ ID NO: 2022 is sufficient to detect the polymorphism at rs10750376.

In some embodiments, the target nucleic acid is at least 10 contiguous nucleic acid molecules of SEQ ID NO: 2023 comprising a non-reference allele at nucleoposition 501 within SEQ ID NO: 2023. In some embodiments, the target nucleic acid is at least 10 contiguous nucleic acid molecules of SEQ ID NO: 2023 comprising a “G” or an “A” allele at nucleoposition 501 within SEQ ID NO: 2023. In some embodiments, detecting the at least 10 contiguous nucleic acid molecules comprising a “G” or an “A” allele at nucleoposition 501 within SEQ ID NO: 2023 is sufficient to detect the polymorphism at rs10932456.

In some embodiments, the target nucleic acid is at least 10 contiguous nucleic acid molecules of SEQ ID NO: 2024 comprising a non-reference allele at nucleoposition 501 within SEQ ID NO: 2024. In some embodiments, the target nucleic acid is at least 10 contiguous nucleic acid molecules of SEQ ID NO: 2024 comprising an “A” or a “G” allele at nucleoposition 501 within SEQ ID NO: 2024. In some embodiments, detecting the at least 10 contiguous nucleic acid molecules comprising an “A” or a “G” allele at nucleoposition 501 within SEQ ID NO: 2024 is sufficient to detect the polymorphism at rs1326860.

In some embodiments, the target nucleic acid is at least 10 contiguous nucleic acid molecules of SEQ ID NO: 2025 comprising a “G” or an “A” allele at nucleoposition 501 within SEQ ID NO: 2025. In some embodiments, detecting the at least 10 contiguous nucleic acid molecules comprising a “G” or an “A” allele at nucleoposition 501 within SEQ ID NO: 2025 is sufficient to detect the polymorphism at rs1528663.

In some embodiments, the target nucleic acid is at least 10 contiguous nucleic acid molecules of SEQ ID NO: 2026 comprising a non-reference allele at nucleoposition 501 within SEQ ID NO: 2026. In some embodiments, the target nucleic acid is at least 10 contiguous nucleic acid molecules of SEQ ID NO: 2026 comprising a “C” or an “A” allele at nucleoposition 501 within SEQ ID NO: 2026. In some embodiments, detecting the at least 10 contiguous nucleic acid molecules comprising a “C” or an “A” allele at nucleoposition 501 within SEQ ID NO: 2026 is sufficient to detect the polymorphism at rs1892231.

In some embodiments, the target nucleic acid is at least 10 contiguous nucleic acid molecules of SEQ ID NO: 2027 comprising a non-reference allele at nucleoposition 501 within SEQ ID NO: 2027. In some embodiments, the target nucleic acid is at least 10 contiguous nucleic acid molecules of SEQ ID NO: 2027 comprising a “G” or an “A” allele at nucleoposition 501 within SEQ ID NO: 2027. In some embodiments, detecting the at least 10 contiguous nucleic acid molecules comprising a “G” or an “A” allele at nucleoposition 501 within SEQ ID NO: 2027 is sufficient to detect the polymorphism at rs951279.

In some embodiments, the target nucleic acid is at least 10 contiguous nucleic acid molecules of SEQ ID NO: 2028 comprising a non-reference allele at nucleoposition 501 within SEQ ID NO: 2028. In some embodiments, the target nucleic acid is at least 10 contiguous nucleic acid molecules of SEQ ID NO: 2028 comprising an “A” or a “G” allele at nucleoposition 501 within SEQ ID NO: 2028. In some embodiments, detecting the at least 10 contiguous nucleic acid molecules comprising an “A” or a “G” allele at nucleoposition 501 within SEQ ID NO: 2028 is sufficient to detect the polymorphism at rs9806914.

In some embodiments, the target nucleic acid is at least 10 contiguous nucleic acid molecules of SEQ ID NO: 2029 comprising a non-reference allele at nucleoposition 501 within SEQ ID NO: 2029. In some embodiments, the target nucleic acid is at least 10 contiguous nucleic acid molecules of SEQ ID NO: 2029 comprising a “C” or an “A” allele at nucleoposition 501 within SEQ ID NO: 2029. In some embodiments, detecting the at least 10 contiguous nucleic acid molecules comprising a “C” or an “A” allele at nucleoposition 501 within SEQ ID NO: 2029 is sufficient to detect the polymorphism at rs7935393.

In some embodiments, the target nucleic acid is at least 10 contiguous nucleic acid molecules of SEQ ID NO: 2030 comprising a non-reference allele at nucleoposition 501 within SEQ ID NO: 2030. In some embodiments, the target nucleic acid is at least 10 contiguous nucleic acid molecules of SEQ ID NO: 2030 comprising a “G” or a “C” allele at nucleoposition 501 within SEQ ID NO: 2030. In some embodiments, detecting the at least 10 contiguous nucleic acid molecules comprising a “G” or a “C” allele at nucleoposition 501 within SEQ ID NO: 2030 is sufficient to detect the polymorphism at rs1690492.

In some embodiments, the target nucleic acid is at least 10 contiguous nucleic acid molecules of SEQ ID NO: 2031 comprising a non-reference allele at nucleoposition 501 within SEQ ID NO: 2031. In some embodiments, the target nucleic acid is at least 10 contiguous nucleic acid molecules of SEQ ID NO: 2031 comprising a “G” or an “A” allele at nucleoposition 501 within SEQ ID NO: 2031. In some embodiments, detecting the at least 10 contiguous nucleic acid molecules comprising a “G” or an “A” allele at nucleoposition 501 within SEQ ID NO: 2031 is sufficient to detect the polymorphism at rs420726.

In some embodiments, the target nucleic acid is at least 10 contiguous nucleic acid molecules of SEQ ID NO: 2032 comprising a non-reference allele at nucleoposition 501 within SEQ ID NO: 2032. In some embodiments, the target nucleic acid is at least 10 contiguous nucleic acid molecules of SEQ ID NO: 2032 comprising a “T” or an “A” allele at nucleoposition 501 within SEQ ID NO: 2032. In some embodiments, detecting the at least 10 contiguous nucleic acid molecules comprising a “T” of an “A” allele at nucleoposition 501 within SEQ ID NO: 2032 is sufficient to detect the polymorphism at rs7759385.

In some embodiments, the target nucleic acid is at least 10 contiguous nucleic acid molecules of SEQ ID NO: 2033 comprising a non-reference allele at nucleoposition 501 within SEQ ID NO: 2033. In some embodiments, the target nucleic acid is at least 10 contiguous nucleic acid molecules of SEQ ID NO: 2033 comprising an “A” or a “G” allele at nucleoposition 501 within SEQ ID NO: 2033. In some embodiments, detecting the at least 10 contiguous nucleic acid molecules comprising an “A” or a “G” allele at nucleoposition 501 within SEQ ID NO: 2033 is sufficient to detect the polymorphism at rs10974900.

In some embodiments, the target nucleic acid is at least 10 contiguous nucleic acid molecules of SEQ ID NO: 2034 comprising a non-reference allele at nucleoposition 501 within SEQ ID NO: 2034. In some embodiments, the target nucleic acid is at least 10 contiguous nucleic acid molecules of SEQ ID NO: 2034 comprising an “A” or a “G” allele at nucleoposition 501 within SEQ ID NO: 2034. In some embodiments, detecting the at least 10 contiguous nucleic acid molecules comprising an “A” or a “G” allele at nucleoposition 501 within SEQ ID NO: 2034 is sufficient to detect the polymorphism at rs1326860.

In some embodiments, the target nucleic acid is at least 10 contiguous nucleic acid molecules of SEQ ID NO: 2035 comprising a non-reference allele at nucleoposition 501 within SEQ ID NO: 2035. In some embodiments, the target nucleic acid is at least 10 contiguous nucleic acid molecules of SEQ ID NO: 2035 comprising a “C” or a “G” allele at nucleoposition 501 within SEQ ID NO: 2035. In some embodiments, detecting the at least 10 contiguous nucleic acid molecules comprising a “C” or a “G” allele at nucleoposition 501 within SEQ ID NO: 2035 is sufficient to detect the polymorphism at rs2548147.

In some embodiments, the target nucleic acid is at least 10 contiguous nucleic acid molecules of SEQ ID NO: 2036 comprising a non-reference allele at nucleoposition 501 within SEQ ID NO: 2036. In some embodiments, the target nucleic acid is at least 10 contiguous nucleic acid molecules of SEQ ID NO: 2036 comprising an “A” or a “G” allele at nucleoposition 501 within SEQ ID NO: 2036. In some embodiments, detecting the at least 10 contiguous nucleic acid molecules comprising an “A” of a “G” allele at nucleoposition 501 within SEQ ID NO: 2036 is sufficient to detect the polymorphism at rs2815844.

In some embodiments, the target nucleic acid is at least 10 contiguous nucleic acid molecules of SEQ ID NO: 2037 comprising a non-reference allele at nucleoposition 501 within SEQ ID NO: 2037. In some embodiments, the target nucleic acid is at least 10 contiguous nucleic acid molecules of SEQ ID NO: 2037 comprising a “G” or an “A” allele at nucleoposition 501 within SEQ ID NO: 2037. In some embodiments, detecting the at least 10 contiguous nucleic acid molecules comprising a “G” or an “A” allele at nucleoposition 501 within SEQ ID NO: 2037 is sufficient to detect the polymorphism at rs889702.

In some embodiments, the target nucleic acid is at least 10 contiguous nucleic acid molecules of SEQ ID NO: 2038 comprising a non-reference allele at nucleoposition 501 within SEQ ID NO: 2038. In some embodiments, the target nucleic acid is at least 10 contiguous nucleic acid molecules of SEQ ID NO: 2038 comprising an “A” or a “G” allele at nucleoposition 501 within SEQ ID NO: 2038. In some embodiments, detecting the at least 10 contiguous nucleic acid molecules comprising an “A” or a “G” allele at nucleoposition 501 within SEQ ID NO: 2038 is sufficient to detect the polymorphism at rs9806914.

In some embodiments, the target nucleic acid is at least 10 contiguous nucleic acid molecules of SEQ ID NO: 2039 comprising a non-reference allele at nucleoposition 501 within SEQ ID NO: 2039. In some embodiments, the target nucleic acid is at least 10 contiguous nucleic acid molecules of SEQ ID NO: 2039 comprising an “A” or a “G” allele at nucleoposition 501 within SEQ ID NO: 2039. In some embodiments, detecting the at least 10 contiguous nucleic acid molecules comprising an “A” or a “G” allele at nucleoposition 501 within SEQ ID NO: 2039 is sufficient to detect the polymorphism at rs6478109.

In some embodiments, the target nucleic acid is at least 10 contiguous nucleic acid molecules of SEQ ID NO: 2040 comprising a non-reference allele at nucleoposition 501 within SEQ ID NO: 2040. In some embodiments, the target nucleic acid is at least 10 contiguous nucleic acid molecules of SEQ ID NO: 2040 comprising a “C” or a “G” allele at nucleoposition 501 within SEQ ID NO: 2040. In some embodiments, detecting the at least 10 contiguous nucleic acid molecules comprising a “C” or a “G” allele at nucleoposition 501 within SEQ ID NO: 2040 is sufficient to detect the polymorphism at rs7278257.

In some embodiments, the target nucleic acid is at least 10 contiguous nucleic acid molecules of SEQ ID NO: 2041 comprising a non-reference allele at nucleoposition 501 within SEQ ID NO: 2041. In some embodiments, the target nucleic acid is at least 10 contiguous nucleic acid molecules of SEQ ID NO: 2041 comprising an “A” or a “G” allele at nucleoposition 501 within SEQ ID NO: 2041. In some embodiments, detecting the at least 10 contiguous nucleic acid molecules comprising an “A” or a “G” allele at nucleoposition 501 within SEQ ID NO: 2041 is sufficient to detect the polymorphism at rs11221332.

In some embodiments, the target nucleic acid is at least 10 contiguous nucleic acid molecules of SEQ ID NO: 2057 comprising a non-reference allele at nucleoposition 501 within SEQ ID NO: 2057. In some embodiments, the target nucleic acid is at least 10 contiguous nucleic acid molecules of SEQ ID NO: 2057 comprising an “A” or a “G” allele at nucleoposition 501 within SEQ ID NO: 2057. In some embodiments, detecting the at least 10 contiguous nucleic acid molecules comprising an “A” or a “G” allele at nucleoposition 501 within SEQ ID NO: 2057 is sufficient to detect the polymorphism at rs56124762.

In some embodiments, the target nucleic acid is at least 10 contiguous nucleic acid molecules of SEQ ID NO: 2058 comprising a non-reference allele at nucleoposition 501 within SEQ ID NO: 2058. In some embodiments, the target nucleic acid is at least 10 contiguous nucleic acid molecules of SEQ ID NO: 2058 comprising an “A” or a “G” allele at nucleoposition 501 within SEQ ID NO: 2058. In some embodiments, detecting the at least 10 contiguous nucleic acid molecules comprising an “A” or a “G” allele at nucleoposition 501 within SEQ ID NO: 2058 is sufficient to detect the polymorphism at rs2070558.

In some embodiments, the target nucleic acid is at least 10 contiguous nucleic acid molecules of SEQ ID NO: 2059 comprising a non-reference allele at nucleoposition 501 within SEQ ID NO: 2059. In some embodiments, the target nucleic acid is at least 10 contiguous nucleic acid molecules of SEQ ID NO: 2059 comprising an “T” or a “C” allele at nucleoposition 501 within SEQ ID NO: 2059. In some embodiments, detecting the at least 10 contiguous nucleic acid molecules comprising an “T” or a “C” allele at nucleoposition 501 within SEQ ID NO: 2059 is sufficient to detect the polymorphism at rs2070561.

In some embodiments, one target nucleic acid (e.g., a polymorphism) is detected with the methods disclosed herein. In some embodiments, at least 2, 3, 4, 5, 6, 7, 8, 9, or 10 target nucleic acids are detected. In some embodiments, the at least 2, 3, 4, 5, 6, 7, 8, 9, or 10 target nucleic acids are detected in a single multiplexed assay. In some embodiments, when 4 target nucleic acids are detected in a sample from subject, 4 unique 3-polymorphism combinations are measured. In a non-limiting example, a sample (e.g., blood or plasma) obtained from a subject is contacted by 4 primer pairs, each primer pair individually adapted to amplify rs6487109, rs56124762, rs1892231, and rs16901748, respectively. A positive, negative, or indeterminate TNFSF15 profile may depend, at least in part, on which of the 3-polymorphism combinations is detected in the sample, and/or whether the genotype is heterozygous or homozygous for the polymorphism. In this example, assaying 4 polymorphism means a total of 4 unique 3-polymorphisms may be detected in the patient sample, which are rs6478109, rs56124762, rs1892231; rs6478109, rs56124762, rs16901748; rs6478109, rs1892231, rs16901748; and rs56124762, rs1892231, rs16901748. Each polymorphism detected may be heterozygous or homozygous.

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B, Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. In some embodiments, the target protein is TL1A, or a binding partner of TL1A such as Death Domain Receptor 3 (DcR3). A target protein may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Non-limiting examples of binding partners to TL1A include DcR3, and Tumor necrosis factor receptor superfamily member 25 (TNR25). Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-I2 antibody, and anti-Cbir1 flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

### 5.4 Therapeutic Agents

### 5.4.1 Antibodies

In one aspect, provided herein are antibodies and antigen-binding fragments. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or 1 amino acid mutations. The humanized light chain variable domain may comprise IGKV3-20 framework with no or fewer than about 10, 9, 8, 7, 6, 5, 4, 3, 2, or 1 amino acid mutations.

In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

In some embodiments, a protein such as an antibody described herein comprises a hydrophobic amino acid. Non-limiting exemplary hydrophobic amino acids include glycine (Gly), proline (Pro), phenylalanine (Phe), alanine (Ala), isoleucine (Ile), leucine (Leu), and valine (Val). In some embodiments, a protein such as an antibody described herein comprises a hydrophilic amino acid. Non-limiting exemplary hydrophilic amino acids include serine (Ser), threonine (Thr), aspartic acid (Asp), glutamic acid (Glu), cysteine (Cys), asparagine (Asn), glutamine (Gln), arginine (Arg), and histidine (His). In some embodiments, a protein such as an antibody described herein comprises an amphipathic amino acid. Non-limiting exemplary amphipathic amino acids include lysine (Lys), tryptophan (Trp), tyrosine (Tyr), and methionine (Met). In some embodiments, a protein such as an antibody described herein comprises an aliphatic amino acid. Non-limiting exemplary aliphatic amino acids include alanine (Ala), isoleucine (Ile), leucine (Leu) and valine (Val). In some embodiments, a protein such as an antibody described herein comprises an aromatic amino acid. Non-limiting exemplary aromatic amino acids include phenylalanine (Phe), tryptophan (Trp), and tyrosine (Tyr). In some embodiments, a protein such as an antibody described herein comprises an acidic amino acid. Non-limiting exemplary acidic amino acids include aspartic acid (Asp) and glutamic acid (Glu). In some embodiments, a protein such as an antibody described herein comprises a basic amino acid. Non-limiting exemplary basic amino acids include arginine (Arg), histidine (His), and lysine (Lys). In some embodiments, a protein such as an antibody described herein comprises a hydroxylic amino acid. Non-limiting exemplary hydroxylic amino acids include serine (Ser) and threonine (Thr). In some embodiments, a protein such as an antibody described herein comprises a sulfur-containing amino acid. Non-limiting exemplary sulfur-containing amino acids include cysteine (Cys) and methionine (Met). In some embodiments, a protein such as an antibody described herein comprises an amidic amino acid. Non-limiting exemplary amidic amino acids include asparagine (Asn) and glutamine (Gln).

In some embodiments, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means within 10% of the stated amount. For instance, an antibody variable region comprising about 80% identity to a reference variable region may comprise 72% to 88% identity to the reference variable region.

In certain aspects, antibodies are described herein that specifically bind to TL1A (Entrez Gene: 9966; UniProtKB: 095150). In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in Tables 15-21, the examples, and the sequences.

### 5.4.1.1 Exemplary Anti-TL1A CDRs

In certain embodiments, an anti-TL1A antibody comprises a HCDR1 as set forth by any one of SEQ ID NOS: 1 or 601-722. In certain embodiments, an anti-TL1A antibody comprises a HCDR2 as set forth by any one of SEQ ID NOS: 2-5 or 723-787. In certain embodiments, an anti-TL1A antibody comprises a HCDR3 as set forth by any one of SEQ ID NOS: 6-9 or 788-842. In certain embodiments, an anti-TL1A antibody comprises a LCDR1 as set forth by any one of SEQ ID NOS: 10 or 843-865. In certain embodiments, an anti-TL1A antibody comprises a LCDR2 as set forth by any one of SEQ ID NOS: 11 or 866-885. In certain embodiments, an anti-TL1A antibody comprises a LCDR3 as set forth by any one of SEQ ID NOS: 12-15 or 886-1101.

In one aspect, provided herein are anti-TL1A antibodies having a HCDR1 comprising any one of SEQ ID NOS: 1 or 601-708, a HCDR2 comprising any one of SEQ ID NOS: 2-5 or 723-774, a HCDR3 comprising any one of SEQ ID NOS: 6-9 or 788-828, a LCDR1 comprising any one of SEQ ID NOS: 10 or 843-852, a LCDR2 comprising any one of SEQ ID NOS: 11 or 866-873, and a LCDR3 comprising any one of SEQ ID NOS: 12-15 or 886-1089.

In one aspect, provided herein are anti-TL1A antibodies having a HCDR1 comprising SEQ ID NO: 709, a HCDR2 comprising SEQ ID NO: 775, a HCDR3 comprising SEQ ID NO: 829, a LCDR1 comprising SEQ ID NO: 853, a LCDR2 comprising SEQ ID NO: 874, and a LCDR3 comprising SEQ ID NO: 1090.

In one aspect, provided herein are anti-TL1A antibodies having a HCDR1 comprising SEQ ID NO: 710, a HCDR2 comprising SEQ ID NO: 776, a HCDR3 comprising SEQ ID NO: 830, a LCDR1 comprising SEQ ID NO: 854, a LCDR2 comprising SEQ ID NO: 875, and a LCDR3 comprising SEQ ID NO: 1091. In one aspect, provided herein are anti-TL1A antibodies having a HCDR1 comprising SEQ ID NO: 711 or 712, a HCDR2 comprising SEQ ID NO: 777 or 778, a HCDR3 comprising SEQ ID NO: 831 or 832, a LCDR1 comprising SEQ ID NO: 855, a LCDR2 comprising SEQ ID NO: 876, and a LCDR3 comprising SEQ ID NO: 1092.

In one aspect, provided herein are anti-TL1A antibodies having a HCDR1 comprising SEQ ID NO: 713, a HCDR2 comprising SEQ ID NO: 779, a HCDR3 comprising SEQ ID NO: 833, a LCDR1 comprising SEQ ID NO: 856, a LCDR2 comprising SEQ ID NO: 877, and a LCDR3 comprising SEQ ID NO: 1093.

In one aspect, provided herein are anti-TL1A antibodies having a HCDR1 comprising SEQ ID NO: 714, a HCDR2 comprising SEQ ID NO: 780, a HCDR3 comprising SEQ ID NO: 834, a LCDR1 comprising SEQ ID NO: 857, a LCDR2 comprising SEQ ID NO: 878, and a LCDR3 comprising SEQ ID NO: 1094.

In one aspect, provided herein are anti-TL1A antibodies having a HCDR1 comprising SEQ ID NO: 715 or 716, a HCDR2 comprising SEQ ID NO: 781, a HCDR3 comprising SEQ ID NO: 835 or 836, a LCDR1 comprising SEQ ID NO: 858 or 859, a LCDR2 comprising SEQ ID NO: 879, and a LCDR3 comprising SEQ ID NO: 1095.

In one aspect, provided herein are anti-TL1A antibodies having a HCDR1 comprising SEQ ID NO: 717, a HCDR2 comprising SEQ ID NO: 782, a HCDR3 comprising SEQ ID NO: 837, a LCDR1 comprising SEQ ID NO: 860, a LCDR2 comprising SEQ ID NO: 880, and a LCDR3 comprising SEQ ID NO: 1096.

In one aspect, provided herein are anti-TL1A antibodies having a HCDR1 comprising SEQ ID NO: 718, a HCDR2 comprising SEQ ID NO: 783, a HCDR3 comprising SEQ ID NO: 838, a LCDR1 comprising SEQ ID NO: 861, a LCDR2 comprising SEQ ID NO: 881, and a LCDR3 comprising SEQ ID NO: 1097.

In one aspect, provided herein are anti-TL1A antibodies having a HCDR1 comprising SEQ ID NO: 719, a HCDR2 comprising SEQ ID NO: 784, a HCDR3 comprising SEQ ID NO: 839, a LCDR1 comprising SEQ ID NO: 862, a LCDR2 comprising SEQ ID NO: 882, and a LCDR3 comprising SEQ ID NO: 1098.

In one aspect, provided herein are anti-TL1A antibodies having a HCDR1 comprising SEQ ID NO: 720, a HCDR2 comprising SEQ ID NO: 785, a HCDR3 comprising SEQ ID NO: 840, a LCDR1 comprising SEQ ID NO: 863, a LCDR2 comprising SEQ ID NO: 883, and a LCDR3 comprising SEQ ID NO: 1099.

In one aspect, provided herein are anti-TL1A antibodies having a HCDR1 comprising SEQ ID NO: 721, a HCDR2 comprising SEQ ID NO: 786, a HCDR3 comprising SEQ ID NO: 841, a LCDR1 comprising SEQ ID NO: 864, a LCDR2 comprising SEQ ID NO: 884, and a LCDR3 comprising SEQ ID NO: 1100.

In one aspect, provided herein are anti-TL1A antibodies having a HCDR1 comprising SEQ ID NO: 722, a HCDR2 comprising SEQ ID NO: 787, a HCDR3 comprising SEQ ID NO: 842, a LCDR1 comprising SEQ ID NO: 865, a LCDR2 comprising SEQ ID NO: 885, and a LCDR3 comprising SEQ ID NO: 1101.

In certain embodiments, an anti-TL1A antibody comprises a HCDR1, HCDR2, HCDR3, LCDR1, LCDR2, LCDR3 selected from Table 15. In certain embodiments, an anti-TL1A antibody comprises the CDRs set forth in antibody A as shown in Table 20. In certain embodiments, an anti-TL1A antibody comprises the CDRs set forth in antibody B as shown in Table 20. In certain embodiments, an anti-TL1A antibody comprises the CDRs set forth in antibody C as shown in Table 20. In certain embodiments, an anti-TL1A antibody comprises the CDRs set forth in antibody D as shown in Table 20. In certain embodiments, an anti-TL1A antibody comprises the CDRs set forth in antibody E as shown in Table 20. In certain embodiments, an anti-TL1A antibody comprises the CDRs set forth in antibody F as shown in Table 20. In certain embodiments, an anti-TL1A antibody comprises the CDRs set forth in antibody G as shown in Table 20. In certain embodiments, an anti-TL1A antibody comprises the CDRs set forth in antibody H as shown in Table 20. In certain embodiments, an anti-TL1A antibody comprises the CDRs set forth in antibody A2 as shown in Table 20. In certain embodiments, an anti-TL1A antibody comprises the CDRs set forth in antibody B2 as shown in Table 20. In certain embodiments, an anti-TL1A antibody comprises the CDRs set forth in antibody C2 as shown in Table 20. In certain embodiments, an anti-TL1A antibody comprises the CDRs set forth in antibody D2 as shown in Table 20. In certain embodiments, an anti-TL1A antibody comprises the CDRs set forth in antibody E2 as shown in Table 20. In certain embodiments, an anti-TL1A antibody comprises the CDRs set forth in antibody F2 as shown in Table 20. In certain embodiments, an anti-TL1A antibody comprises the CDRs set forth in antibody G2 as shown in Table 20. In certain embodiments, an anti-TL1A antibody comprises the CDRs set forth in antibody H2 as shown in Table 20. In certain embodiments, an anti-TL1A antibody comprises the CDRs set forth in antibody I as shown in Table 20. In certain embodiments, an anti-TL1A antibody comprises the CDRs set forth in antibody I2 as shown in Table 20. In certain embodiments, an anti-TL1A antibody comprises the CDRs set forth in antibody M1 as shown in Table 15. In certain embodiments, an anti-TL1A antibody comprises the CDRs set forth in antibody M2 as shown in Table 15. In certain embodiments, an anti-TL1A antibody comprises the CDRs set forth in antibody M3 as shown in Table 15. In certain embodiments, an anti-TL1A antibody comprises the CDRs set forth in antibody M4 as shown in Table 15. In certain embodiments, an anti-TL1A antibody comprises the CDRs set forth in antibody M5 as shown in Table 15. In certain embodiments, an anti-TL1A antibody comprises the CDRs set forth in antibody M6 as shown in Table 15. In certain embodiments, an anti-TL1A antibody comprises the CDRs set forth in antibody M7 as shown in Table 15. In certain embodiments, an anti-TL1A antibody comprises the CDRs set forth in antibody M8 as shown in Table 15. In certain embodiments, an anti-TL1A antibody comprises the CDRs set forth in antibody M9 as shown in Table 15. In certain embodiments, an anti-TL1A antibody comprises the CDRs set forth in antibody M10 as shown in Table 15. In certain embodiments, an anti-TL1A antibody comprises the CDRs set forth in antibody M11 as shown in Table 15. In certain embodiments, an anti-TL1A antibody comprises the CDRs set forth in antibody M12 as shown in Table 15. In certain embodiments, an anti-TL1A antibody comprises a P HCDR1 (any one of SEQ ID NOS: 1 or 601-708), a P HCDR2 (any one of SEQ ID NOS: 2-5 or 723-774), a P HCDR3 (any one of SEQ ID NOS: 6-9 or 788-828), a P LCDR1 (any one of SEQ ID NOS: 10 or 843-852), a P LCDR2 (any one of SEQ ID NOS: 11 or 866-873), and a P LCDR3 (any one of SEQ ID NOS: 12-15 or 886-1089) as shown in Table 15.

In certain embodiments, an anti-TL1A antibody comprises the CDRs set forth in any one of the antibodies in Table 10. In certain embodiments, an anti-TL1A antibody comprises the CDRs set forth in any one of the heavy chain variable regions in Table 16. In certain embodiments, an anti-TL1A antibody comprises the CDRs set forth in any one of the light chain variable regions in Table 17. The CDRs may be defined by the Aho or Kabat, Chothia, or IMGT method. In certain embodiments, an anti-TL1A antibody comprises the CDRs set forth in antibody A217. In certain embodiments, an anti-TL1A antibody comprises the CDRs set forth in antibody A220. In certain embodiments, an anti-TL1A antibody comprises the CDRs set forth in antibody A223. In certain embodiments, an anti-TL1A antibody comprises the CDRs set forth in antibody A219. In certain embodiments, an anti-TL1A antibody comprises the CDRs set forth in antibody A221. In certain embodiments, an anti-TL1A antibody comprises the CDRs set forth in antibody A200. In certain embodiments, an anti-TL1A antibody comprises the CDRs set forth in antibody A213. In certain embodiments, an anti-TL1A antibody comprises the CDRs set forth in antibody A212. In certain embodiments, an anti-TL1A antibody comprises the CDRs set forth in antibody A107. In certain embodiments, an anti-TL1A antibody comprises the CDRs set forth in antibody A205. In certain embodiments, an anti-TL1A antibody comprises the CDRs set forth in antibody A211. In certain embodiments, an anti-TL1A antibody comprises the CDRs set forth in antibody A199. In certain embodiments, an anti-TL1A antibody comprises the CDRs set forth in antibody A15. In certain embodiments, an anti-TL1A antibody comprises the CDRs set forth in antibody A30. In certain embodiments, an anti-TL1A antibody comprises the CDRs set forth in antibody A100. In certain embodiments, an anti-TL1A antibody comprises the CDRs set forth in antibody A181. In certain embodiments, an anti-TL1A antibody comprises the CDRs set forth in antibody A129. In certain embodiments, an anti-TL1A antibody comprises the CDRs set forth in antibody A214. In certain embodiments, an anti-TL1A antibody comprises the CDRs set forth in antibody A216. In certain embodiments, an anti-TL1A antibody comprises the CDRs set forth in antibody A122. In certain embodiments, an anti-TL1A antibody comprises the CDRs set forth in antibody A222. In certain embodiments, an anti-TL1A antibody comprises the CDRs set forth in antibody A188. In certain embodiments, an anti-TL1A antibody comprises the CDRs set forth in antibody A203. In certain embodiments, an anti-TL1A antibody comprises the CDRs set forth in antibody A147. In certain embodiments, an anti-TL1A antibody comprises the CDRs set forth in antibody A127. In certain embodiments, an anti-TL1A antibody comprises the CDRs set forth in antibody A126. In certain embodiments, an anti-TL1A antibody comprises the CDRs set forth in antibody A160. In certain embodiments, an anti-TL1A antibody comprises the CDRs set forth in antibody A157. In certain embodiments, an anti-TL1A antibody comprises the CDRs set forth in antibody A159. In certain embodiments, an anti-TL1A antibody comprises the CDRs set forth in antibody A218. In certain embodiments, an anti-TL1A antibody comprises the CDRs set forth in antibody A158. In certain embodiments, an anti-TL1A antibody comprises the CDRs set forth in antibody A125. In certain embodiments, an anti-TL1A antibody comprises the CDRs set forth in antibody A103. In certain embodiments, an anti-TL1A antibody comprises the CDRs set forth in antibody A64. In certain embodiments, an anti-TL1A antibody comprises the CDRs set forth in antibody A67. In certain embodiments, an anti-TL1A antibody comprises the CDRs set forth in antibody A138. In certain embodiments, an anti-TL1A antibody comprises the CDRs set forth in antibody A68. In certain embodiments, an anti-TL1A antibody comprises the CDRs set forth in antibody A94. In certain embodiments, an anti-TL1A antibody comprises the CDRs set forth in antibody A110. In certain embodiments, an anti-TL1A antibody comprises the CDRs set forth in antibody A197. In certain embodiments, an anti-TL1A antibody comprises the CDRs set forth in antibody A112. In certain embodiments, an anti-TL1A antibody comprises the CDRs set forth in antibody A169. In certain embodiments, an anti-TL1A antibody comprises the CDRs set forth in antibody A173. In certain embodiments, an anti-TL1A antibody comprises the CDRs set forth in antibody A179. In certain embodiments, an anti-TL1A antibody comprises the CDRs set forth in antibody A148. In certain embodiments, an anti-TL1A antibody comprises the CDRs set forth in antibody A115. In certain embodiments, an anti-TL1A antibody comprises the CDRs set forth in antibody A149. In certain embodiments, an anti-TL1A antibody comprises the CDRs set forth in antibody A134. In certain embodiments, an anti-TL1A antibody comprises the CDRs set forth in antibody A113. In certain embodiments, an anti-TL1A antibody comprises the CDRs set forth in antibody A151. In certain embodiments, an anti-TL1A antibody comprises the CDRs set forth in antibody A96. In certain embodiments, an anti-TL1A antibody comprises the CDRs set forth in antibody A132. In certain embodiments, an anti-TL1A antibody comprises the CDRs set forth in antibody A196. In certain embodiments, an anti-TL1A antibody comprises the CDRs set forth in antibody A172. In certain embodiments, an anti-TL1A antibody comprises the CDRs set forth in antibody A75. In certain embodiments, an anti-TL1A antibody comprises the CDRs set forth in antibody A174. In certain embodiments, an anti-TL1A antibody comprises the CDRs set forth in antibody A109. In certain embodiments, an anti-TL1A antibody comprises the CDRs set forth in antibody A198. In certain embodiments, an anti-TL1A antibody comprises the CDRs set forth in antibody A170. In certain embodiments, an anti-TL1A antibody comprises the CDRs set forth in antibody M1. In certain embodiments, an anti-TL1A antibody comprises the CDRs set forth in antibody M2. In certain embodiments, an anti-TL1A antibody comprises the CDRs set forth in antibody M3. In certain embodiments, an anti-TL1A antibody comprises the CDRs set forth in antibody M4. In certain embodiments, an anti-TL1A antibody comprises the CDRs set forth in antibody M5. In certain embodiments, an anti-TL1A antibody comprises the CDRs set forth in antibody M6. In certain embodiments, an anti-TL1A antibody comprises the CDRs set forth in antibody M7. In certain embodiments, an anti-TL1A antibody comprises the CDRs set forth in antibody M8. In certain embodiments, an anti-TL1A antibody comprises the CDRs set forth in antibody M9. In certain embodiments, an anti-TL1A antibody comprises the CDRs set forth in antibody M10. In certain embodiments, an anti-TL1A antibody comprises the CDRs set forth in antibody M11. In certain embodiments, an anti-TL1A antibody comprises the CDRs set forth in antibody M12.

### 5.4.1.2 Exemplary Anti-TL1A Framework Regions

Tables 16-18 and Table 21 provides exemplary framework and variable region sequences. In some embodiments, an anti-TL1A antibody comprises a HC FR1 of Table 19 (SEQ ID NO: 304), a HC FR2 of Table 19 (any one of SEQ ID NOS: 305, 313, 1317), a HC FR3 of Table 19 (any one of SEQ ID NOS: 306, 307, 314, 315, 1318-1323), a HC FR4 of Table 19 (SEQ ID NO: 308), a LC FR1 of Table 19 (SEQ ID NO: 309), a LC FR2 of Table 19 (SEQ ID NO: 310 or 1324), a LC FR3 of Table 19 (SEQ ID NO: 311), and a LC FR4 of Table 19 (SEQ ID NO: 312).

In some embodiments, an anti-TL1A antibody comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Aho or Kabat numbering. In some embodiments, X2 is at position 45 of IGHV1-46*02 as determined by Aho or Kabat numbering. In some embodiments, X3 is at position 47 of IGHV1-46*02 as determined by Aho or Kabat numbering. In some embodiments, X4 is at position 55 of IGHV1-46*02 as determined by Aho or Kabat numbering. In some embodiments, X5 is at position 78 of IGHV1-46*02 as determined by Aho or Kabat numbering. In some embodiments, X6 is at position 80 of IGHV1-46*02 as determined by Aho or Kabat numbering. In some embodiments, X7 is at position 82 of IGHV1-46*02 as determined by Aho or Kabat numbering. In some embodiments, X8 is at position 89 of IGHV1-46*02 as determined by Aho or Kabat numbering. In some embodiments, X9 is at position 91 of IGHV1-46*02 as determined by Aho or Kabat numbering.

In one aspect, provided herein is a first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 301. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. As used herein, any combination of a group, such as (a) to (uu), includes at least about two or more items from the group, e.g., any combination of a group of (a) to (uu) includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and up to 47 or all of the members of the group. (30) The anti-TL1A of any one of embodiments 1-28, comprising a (i) human IgG4 Fc region or (ii) a human IgG4 Fc region comprising (a) S228P, (b) S228P and L235E, or (c) S228P, F234A, and L235A, per Kabat numbering. (31) The anti-TL1A of any one of embodiments 1-28, comprising a human IgG2 Fc region; IgG2-IgG4 cross-subclass Fc region; IgG2-IgG3 cross-subclass Fe region; IgG2 comprising H268Q, V309L, A330S, P331S (IgG2m4); or IgG2 comprising V234A, G237A, P238S, H268A, V309L, A330S, P331S (IgG2σ). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Aho or Kabat numbering. In some embodiments, X2 is at position 45 of IGHV1-46*02 as determined by Aho or Kabat numbering. In some embodiments, X3 is at position 47 of IGHV1-46*02 as determined by Aho or Kabat numbering. In some embodiments, X4 is at position 55 of IGHV1-46*02 as determined by Aho or Kabat numbering. In some embodiments, X5 is at position 78 of IGHV1-46*02 as determined by Aho or Kabat numbering. In some embodiments, X6 is at position 80 of IGHV1-46*02 as determined by Aho or Kabat numbering. In some embodiments, X7 is at position 82 of IGHV1-46*02 as determined by Aho or Kabat numbering. In some embodiments, X8 is at position 89 of IGHV1-46*02 as determined by Aho or Kabat numbering. In some embodiments, X9 is at position 91 of IGHV1-46*02 as determined by Aho or Kabat numbering.

In one aspect, provided herein is another first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 302. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. (30) The anti-TL1A of any one of embodiments 1-28, comprising a (i) human IgG4 Fc region or (ii) a human IgG4 Fc region comprising (a) S228P and L235E, or (b) S228P, F234A, and L235A, per Kabat numbering. (31) The anti-TL1A of any one of embodiments 1-28, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK). In some cases, X10 is L. In some cases, X10 is P. In some cases, X11 is L. In some cases, X11 is W. In some embodiments, X10 is at position 54 of IGKV3-20*01 as determined by Aho or Kabat numbering. In some embodiments, X11 is at position 55 of IGKV3-20*01 as determined by Aho or Kabat numbering.

In some embodiments, an anti-TL1A antibody comprises a heavy chain framework comprising IGHV1-46*02. In some embodiments, an anti-TL1A antibody comprises a heavy chain framework comprising a variant of IGHV1-46*02 comprising between about 1 and about 20 amino acid substitutions from SEQ ID NO: 316. In some embodiments, an anti-TL1A antibody comprises a heavy chain framework comprising a variant of IGHV1-46*02 comprising between about 1 and about 9 amino acid substitutions from SEQ ID NO: 316. In some embodiments, an anti-TL1A antibody comprises a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. In some cases, the heavy chain framework substitution comprises Q1E, as determined by Aho or Kabat numbering. In some cases, the heavy chain framework substitution comprises R45K, as determined by Aho or Kabat numbering. In some cases, the heavy chain framework substitution comprises A47R, as determined by Aho or Kabat numbering. In some cases, the heavy chain framework substitution comprises M55I, as determined by Aho or Kabat numbering. In some cases, the heavy chain framework substitution comprises V78A, as determined by Aho or Kabat numbering. In some cases, the heavy chain framework substitution comprises M80I, as determined by Aho or Kabat numbering. In some cases, the heavy chain framework substitution comprises R82T, as determined by Aho or Kabat numbering. In some cases, the heavy chain framework substitution comprises V89A, as determined by Aho or Kabat numbering. In some cases, the heavy chain framework substitution comprises M91L, as determined by Aho or Kabat numbering.

In some embodiments, an anti-TL1A antibody comprises a light chain framework comprising IGKV3-20*01. In some embodiments, an anti-TL1A antibody comprises a variant of IGKV3-20*01 comprising between about 1 and about 20 amino acid substitutions from SEQ ID NO: 317. In some embodiments, an anti-TL1A antibody comprises a variant of IGKV3-20*01 comprising about 1 amino acid substitution from SEQ ID NO: 317. In some embodiments, an anti-TL1A antibody comprises a light chain framework comprising a variant of IGKV3-20*01 comprising about 2 amino acid substitutions from SEQ ID NO: 317. In some embodiments, an anti-TL1A antibody comprises a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. In some cases, the light chain framework substitution comprises Q1E, as determined by Aho or Kabat numbering. In some cases, the light chain framework substitution comprises R45K, as determined by Aho or Kabat numbering.

In some embodiments, an anti-TL1A antibody comprises a heavy chain FR1 as set forth by SEQ ID NO: 304. In some embodiments, an anti-TL1A antibody comprises a heavy chain FR2 as set forth by SEQ ID NO: 305. In some embodiments, an anti-TL1A antibody comprises a heavy chain FR2 as set forth by SEQ ID NO: 313. In some embodiments, an anti-TL1A antibody comprises a heavy chain FR2 as set forth by SEQ ID NO: 1317. In some embodiments, an anti-TL1A antibody comprises a heavy chain FR3 as set forth by SEQ ID NO: 306. In some embodiments, an anti-TL1A antibody comprises a heavy chain FR3 as set forth by SEQ ID NO: 307. In some embodiments, an anti-TL1A antibody comprises a heavy chain FR3 as set forth by SEQ ID NO: 314. In some embodiments, an anti-TL1A antibody comprises a heavy chain FR3 as set forth by SEQ ID NO: 315. In some embodiments, an anti-TL1A antibody comprises a heavy chain FR3 as set forth by SEQ ID NO: 1318. In some embodiments, an anti-TL1A antibody comprises a heavy chain FR3 as set forth by SEQ ID NO: 1319. In some embodiments, an anti-TL1A antibody comprises a heavy chain FR3 as set forth by SEQ ID NO: 1320. In some embodiments, an anti-TL1A antibody comprises a heavy chain FR3 as set forth by SEQ ID NO: 1321. In some embodiments, an anti-TL1A antibody comprises a heavy chain FR3 as set forth by SEQ ID NO: 1322. In some embodiments, an anti-TL1A antibody comprises a heavy chain FR3 as set forth by SEQ ID NO: 1323. In some embodiments, an anti-TL1A antibody comprises a heavy chain FR4 as set forth by SEQ ID NO: 308. In some embodiments, an anti-TL1A antibody comprises a light chain FR1 as set forth by SEQ ID NO: 309. In some embodiments, an anti-TL1A antibody comprises a light chain FR2 as set forth by SEQ ID NO: 310. In some embodiments, an anti-TL1A antibody comprises a light chain FR2 as set forth by SEQ ID NO: 1324. In some embodiments, an anti-TL1A antibody comprises a light chain FR3 as set forth by SEQ ID NO: 311. In some embodiments, an anti-TL1A antibody comprises a light chain FR3 as set forth by SEQ ID NO: 1325. In some embodiments, an anti-TL1A antibody comprises a light chain FR4 as set forth by SEQ ID NO: 312.

In certain embodiments, an anti-TL1A antibody comprises the framework regions set forth in any one of the antibodies in Table 16 and Table 17, wherein the framework regions are defined by the Aho or Kabat, Chothia, or IMGT method. In certain embodiments, an anti-TL1A antibody comprises the framework regions set forth in antibody A217. In certain embodiments, an anti-TL1A antibody comprises the framework regions set forth in antibody A220. In certain embodiments, an anti-TL1A antibody comprises the framework regions set forth in antibody A223. In certain embodiments, an anti-TL1A antibody comprises the framework regions set forth in antibody A219. In certain embodiments, an anti-TL1A antibody comprises the framework regions set forth in antibody A221. In certain embodiments, an anti-TL1A antibody comprises the framework regions set forth in antibody A200. In certain embodiments, an anti-TL1A antibody comprises the framework regions set forth in antibody A213. In certain embodiments, an anti-TL1A antibody comprises the framework regions set forth in antibody A212. In certain embodiments, an anti-TL1A antibody comprises the framework regions set forth in antibody A107. In certain embodiments, an anti-TL1A antibody comprises the framework regions set forth in antibody A205. In certain embodiments, an anti-TL1A antibody comprises the framework regions set forth in antibody A211. In certain embodiments, an anti-TL1A antibody comprises the framework regions set forth in antibody A199. In certain embodiments, an anti-TL1A antibody comprises the framework regions set forth in antibody A15. In certain embodiments, an anti-TL1A antibody comprises the framework regions set forth in antibody A30. In certain embodiments, an anti-TL1A antibody comprises the framework regions set forth in antibody A100. In certain embodiments, an anti-TL1A antibody comprises the framework regions set forth in antibody A181. In certain embodiments, an anti-TL1A antibody comprises the framework regions set forth in antibody A129. In certain embodiments, an anti-TL1A antibody comprises the framework regions set forth in antibody A214. In certain embodiments, an anti-TL1A antibody comprises the framework regions set forth in antibody A216. In certain embodiments, an anti-TL1A antibody comprises the framework regions set forth in antibody A122. In certain embodiments, an anti-TL1A antibody comprises the framework regions set forth in antibody A222. In certain embodiments, an anti-TL1A antibody comprises the framework regions set forth in antibody A188. In certain embodiments, an anti-TL1A antibody comprises the framework regions set forth in antibody A203. In certain embodiments, an anti-TL1A antibody comprises the framework regions set forth in antibody A147. In certain embodiments, an anti-TL1A antibody comprises the framework regions set forth in antibody A127. In certain embodiments, an anti-TL1A antibody comprises the framework regions set forth in antibody A126. In certain embodiments, an anti-TL1A antibody comprises the framework regions set forth in antibody A160. In certain embodiments, an anti-TL1A antibody comprises the framework regions set forth in antibody A157. In certain embodiments, an anti-TL1A antibody comprises the framework regions set forth in antibody A159. In certain embodiments, an anti-TL1A antibody comprises the framework regions set forth in antibody A218. In certain embodiments, an anti-TL1A antibody comprises the framework regions set forth in antibody A158. In certain embodiments, an anti-TL1A antibody comprises the framework regions set forth in antibody A125. In certain embodiments, an anti-TL1A antibody comprises the framework regions set forth in antibody A103. In certain embodiments, an anti-TL1A antibody comprises the framework regions set forth in antibody A64. In certain embodiments, an anti-TL1A antibody comprises the framework regions set forth in antibody A67. In certain embodiments, an anti-TL1A antibody comprises the framework regions set forth in antibody A138. In certain embodiments, an anti-TL1A antibody comprises the framework regions set forth in antibody A68. In certain embodiments, an anti-TL1A antibody comprises the framework regions set forth in antibody A94. In certain embodiments, an anti-TL1A antibody comprises the framework regions set forth in antibody A110. In certain embodiments, an anti-TL1A antibody comprises the framework regions set forth in antibody A197. In certain embodiments, an anti-TL1A antibody comprises the framework regions set forth in antibody A112. In certain embodiments, an anti-TL1A antibody comprises the framework regions set forth in antibody A169. In certain embodiments, an anti-TL1A antibody comprises the framework regions set forth in antibody A173. In certain embodiments, an anti-TL1A antibody comprises the framework regions set forth in antibody A179. In certain embodiments, an anti-TL1A antibody comprises the framework regions set forth in antibody A148. In certain embodiments, an anti-TL1A antibody comprises the framework regions set forth in antibody A115. In certain embodiments, an anti-TL1A antibody comprises the framework regions set forth in antibody A149. In certain embodiments, an anti-TL1A antibody comprises the framework regions set forth in antibody A134. In certain embodiments, an anti-TL1A antibody comprises the framework regions set forth in antibody A113. In certain embodiments, an anti-TL1A antibody comprises the framework regions set forth in antibody A151. In certain embodiments, an anti-TL1A antibody comprises the framework regions set forth in antibody A96. In certain embodiments, an anti-TL1A antibody comprises the framework regions set forth in antibody A132. In certain embodiments, an anti-TL1A antibody comprises the framework regions set forth in antibody A196. In certain embodiments, an anti-TL1A antibody comprises the framework regions set forth in antibody A172. In certain embodiments, an anti-TL1A antibody comprises the framework regions set forth in antibody A75. In certain embodiments, an anti-TL1A antibody comprises the framework regions set forth in antibody A174. In certain embodiments, an anti-TL1A antibody comprises the framework regions set forth in antibody A109. In certain embodiments, an anti-TL1A antibody comprises the framework regions set forth in antibody A198. In certain embodiments, an anti-TL1A antibody comprises the framework regions set forth in antibody A170.

In certain embodiments, an anti-TL1A antibody comprises the heavy chain framework regions set forth in any one of the antibodies in Table 16, and the light chain framework regions set forth in any one of the antibodies in Table 17, wherein the framework regions are defined by the Aho or Kabat, Chothia, or IMGT method.

### 5.4.1.3 Exemplary Anti-TL1A Variable Regions

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 101-135; and a light chain variable region at least about 85%, 90%, 91%, 92%, 93%, 94%, 95%, 96%, 97%, 98%, 99%, or 100% identical to any one of SEQ ID NOS: 201-206.

Further provided herein is a first embodiment of an anti-TL1A antibody comprising a heavy chain variable region and a light chain variable region. Non-limiting additional embodiments include: (Embodiment 2) The anti-TL1A antibody of embodiment 1, wherein the heavy chain variable region comprises SEQ ID NO: 101. (Embodiment 3) The anti-TL1A antibody of embodiment 1, wherein the heavy chain variable region comprises a sequence at least about 90%, 95%, 96%, 97%, 98%, 99%, or 100% identical to SEQ ID NO: 101. (Embodiment 4) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 101. (Embodiment 5) The anti-TL1A antibody of embodiment 1, wherein the heavy chain variable region comprises SEQ ID NO: 102. (Embodiment 6) The anti-TL1A antibody of embodiment 1, wherein the heavy chain variable region comprises a sequence at least about 90%, 95%, 96%, 97%, 98%, 99%, or 100% identical to SEQ ID NO: 102. (Embodiment 7) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 102. (Embodiment 8) The anti-TL1A antibody of embodiment 1, wherein the heavy chain variable region comprises SEQ ID NO: 103. (Embodiment 9) The anti-TL1A antibody of embodiment 1, wherein the heavy chain variable region comprises a sequence at least about 90%, 95%, 96%, 97%, 98%, 99%, or 100% identical to SEQ ID NO: 103. (Embodiment 10) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 103.

(Embodiment 11) The anti-TL1A antibody of embodiment 1, wherein the heavy chain variable region comprises SEQ ID NO: 104. (Embodiment 12) The anti-TL1A antibody of embodiment 1, wherein the heavy chain variable region comprises a sequence at least about 90%, 95%, 96%, 97%, 98%, 99%, or 100% identical to SEQ ID NO: 104. (Embodiment 13) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 104. (Embodiment 14) The anti-TL1A antibody of embodiment 1, wherein the heavy chain variable region comprises SEQ ID NO: 105. (Embodiment 15) The anti-TL1A antibody of embodiment 1, wherein the heavy chain variable region comprises a sequence at least about 90%, 95%, 96%, 97%, 98%, 99%, or 100% identical to SEQ ID NO: 105. (Embodiment 16) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 105. (Embodiment 17) The anti-TL1A antibody of embodiment 1, wherein the heavy chain variable region comprises SEQ ID NO: 106. (Embodiment 18) The anti-TL1A antibody of embodiment 1, wherein the heavy chain variable region comprises a sequence at least about 90%, 95%, 96%, 97%, 98%, 99%, or 100% identical to SEQ ID NO: 106. (Embodiment 19) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 106. (Embodiment 20) The anti-TL1A antibody of embodiment 1, wherein the heavy chain variable region comprises SEQ ID NO: 107. (Embodiment 21) The anti-TL1A antibody of embodiment 1, wherein the heavy chain variable region comprises a sequence at least about 90%, 95%, 96%, 97%, 98%, 99%, or 100% identical to SEQ ID NO: 107. (Embodiment 22) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 107.

(Embodiment 23) The anti-TL1A antibody of embodiment 1, wherein the heavy chain variable region comprises SEQ ID NO: 108. (Embodiment 24) The anti-TL1A antibody of embodiment 1, wherein the heavy chain variable region comprises a sequence at least about 90%, 95%, 96%, 97%, 98%, 99%, or 100% identical to SEQ ID NO: 108. (Embodiment 25) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 108. (Embodiment 26) The anti-TL1A antibody of embodiment 1, wherein the heavy chain variable region comprises SEQ ID NO: 109. (Embodiment 27) The anti-TL1A antibody of embodiment 1, wherein the heavy chain variable region comprises a sequence at least about 90%, 95%, 96%, 97%, 98%, 99%, or 100% identical to SEQ ID NO: 109. (Embodiment 28) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 109. (Embodiment 29) The anti-TL1A antibody of embodiment 1, wherein the heavy chain variable region comprises SEQ ID NO: 110. (Embodiment 30) The anti-TL1A antibody of embodiment 1, wherein the heavy chain variable region comprises a sequence at least about 90%, 95%, 96%, 97%, 98%, 99%, or 100% identical to SEQ ID NO: 110. (Embodiment 31) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 110. (Embodiment 32) The anti-TL1A antibody of embodiment 1, wherein the heavy chain variable region comprises SEQ ID NO: 111. (Embodiment 33) The anti-TL1A antibody of embodiment 1, wherein the heavy chain variable region comprises a sequence at least about 90%, 95%, 96%, 97%, 98%, 99%, or 100% identical to SEQ ID NO: 111. (Embodiment 34) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 111. (Embodiment 35) The anti-TL1A antibody of embodiment 1, wherein the heavy chain variable region comprises SEQ ID NO: 112. (Embodiment 36) The anti-TL1A antibody of embodiment 1, wherein the heavy chain variable region comprises a sequence at least about 90%, 95%, 96%, 97%, 98%, 99%, or 100% identical to SEQ ID NO: 112. (Embodiment 37) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 112. (Embodiment 38) The anti-TL1A antibody of embodiment 1, wherein the heavy chain variable region comprises SEQ ID NO: 113. (Embodiment 39) The anti-TL1A antibody of embodiment 1, wherein the heavy chain variable region comprises a sequence at least about 90%, 95%, 96%, 97%, 98%, 99%, or 100% identical to SEQ ID NO: 113. (Embodiment 40) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 113.

(Embodiment 41) The anti-TL1A antibody of embodiment 1, wherein the heavy chain variable region comprises SEQ ID NO: 114. (Embodiment 42) The anti-TL1A antibody of embodiment 1, wherein the heavy chain variable region comprises a sequence at least about 90%, 95%, 96%, 97%, 98%, 99%, or 100% identical to SEQ ID NO: 114. (Embodiment 43) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 114. (Embodiment 44) The anti-TL1A antibody of embodiment 1, wherein the heavy chain variable region comprises SEQ ID NO: 115. (Embodiment 45) The anti-TL1A antibody of embodiment 1, wherein the heavy chain variable region comprises a sequence at least about 90%, 95%, 96%, 97%, 98%, 99%, or 100% identical to SEQ ID NO: 115. (Embodiment 46) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 115. (Embodiment 47) The anti-TL1A antibody of embodiment 1, wherein the heavy chain variable region comprises SEQ ID NO: 116. (Embodiment 48) The anti-TL1A antibody of embodiment 1, wherein the heavy chain variable region comprises a sequence at least about 90%, 95%, 96%, 97%, 98%, 99%, or 100% identical to SEQ ID NO: 116. (Embodiment 49) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 116. (Embodiment 50) The anti-TL1A antibody of embodiment 1, wherein the heavy chain variable region comprises SEQ ID NO: 117. (Embodiment 51) The anti-TL1A antibody of embodiment 1, wherein the heavy chain variable region comprises a sequence at least about 90%, 95%, 96%, 97%, 98%, 99%, or 100% identical to SEQ ID NO: 117. (Embodiment 52) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 117.

(Embodiment 53) The anti-TL1A antibody of embodiment 1, wherein the heavy chain variable region comprises SEQ ID NO: 118. (Embodiment 54) The anti-TL1A antibody of embodiment 1, wherein the heavy chain variable region comprises a sequence at least about 90%, 95%, 96%, 97%, 98%, 99%, or 100% identical to SEQ ID NO: 118. (Embodiment 55) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 118. (Embodiment 56) The anti-TL1A antibody of embodiment 1, wherein the heavy chain variable region comprises SEQ ID NO: 119. (Embodiment 57) The anti-TL1A antibody of embodiment 1, wherein the heavy chain variable region comprises a sequence at least about 90%, 95%, 96%, 97%, 98%, 99%, or 100% identical to SEQ ID NO: 119. (Embodiment 58) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 119. (Embodiment 59) The anti-TL1A antibody of embodiment 1, wherein the heavy chain variable region comprises SEQ ID NO: 120. (Embodiment 60) The anti-TL1A antibody of embodiment 1, wherein the heavy chain variable region comprises a sequence at least about 90%, 95%, 96%, 97%, 98%, 99%, or 100% identical to SEQ ID NO: 120. (Embodiment 61) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 120.

(Embodiment 62) The anti-TL1A antibody of embodiment 1, wherein the heavy chain variable region comprises SEQ ID NO: 121. (Embodiment 63) The anti-TL1A antibody of embodiment 1, wherein the heavy chain variable region comprises a sequence at least about 90%, 95%, 96%, 97%, 98%, 99%, or 100% identical to SEQ ID NO: 121. (Embodiment 64) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 121. (Embodiment 65) The anti-TL1A antibody of embodiment 1, wherein the heavy chain variable region comprises SEQ ID NO: 122. (Embodiment 66) The anti-TL1A antibody of embodiment 1, wherein the heavy chain variable region comprises a sequence at least about 90%, 95%, 96%, 97%, 98%, 99%, or 100% identical to SEQ ID NO: 122. (Embodiment 67) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 122. (Embodiment 68) The anti-TL1A antibody of embodiment 1, wherein the heavy chain variable region comprises SEQ ID NO: 123. (Embodiment 69) The anti-TL1A antibody of embodiment 1, wherein the heavy chain variable region comprises a sequence at least about 90%, 95%, 96%, 97%, 98%, 99%, or 100% identical to SEQ ID NO: 123. (Embodiment 70) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 123. (Embodiment 71) The anti-TL1A antibody of embodiment 1, wherein the heavy chain variable region comprises SEQ ID NO: 124. (Embodiment 72) The anti-TL1A antibody of embodiment 1, wherein the heavy chain variable region comprises a sequence at least about 90%, 95%, 96%, 97%, 98%, 99%, or 100% identical to SEQ ID NO: 124. (Embodiment 73) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 124.

(Embodiment 74) The anti-TL1A antibody of embodiment 1, wherein the heavy chain variable region comprises SEQ ID NO: 125. (Embodiment 75) The anti-TL1A antibody of embodiment 1, wherein the heavy chain variable region comprises a sequence at least about 90%, 95%, 96%, 97%, 98%, 99%, or 100% identical to SEQ ID NO: 125. (Embodiment 76) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 125. (Embodiment 77) The anti-TL1A antibody of embodiment 1, wherein the heavy chain variable region comprises SEQ ID NO: 126. (Embodiment 78) The anti-TL1A antibody of embodiment 1, wherein the heavy chain variable region comprises a sequence at least about 90%, 95%, 96%, 97%, 98%, 99%, or 100% identical to SEQ ID NO: 126. (Embodiment 79) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 126. (Embodiment 80) The anti-TL1A antibody of embodiment 1, wherein the heavy chain variable region comprises SEQ ID NO: 127. (Embodiment 81) The anti-TL1A antibody of embodiment 1, wherein the heavy chain variable region comprises a sequence at least about 90%, 95%, 96%, 97%, 98%, 99%, or 100% identical to SEQ ID NO: 127. (Embodiment 82) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 127. (Embodiment 83) The anti-TL1A antibody of embodiment 1, wherein the heavy chain variable region comprises SEQ ID NO: 128. (Embodiment 84) The anti-TL1A antibody of embodiment 1, wherein the heavy chain variable region comprises a sequence at least about 90%, 95%, 96%, 97%, 98%, 99%, or 100% identical to SEQ ID NO: 128. (Embodiment 85) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 128.

(Embodiment 86) The anti-TL1A antibody of embodiment 1, wherein the heavy chain variable region comprises SEQ ID NO: 129. (Embodiment 87) The anti-TL1A antibody of embodiment 1, wherein the heavy chain variable region comprises a sequence at least about 90%, 95%, 96%, 97%, 98%, 99%, or 100% identical to SEQ ID NO: 129. (Embodiment 88) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 129. (Embodiment 89) The anti-TL1A antibody of embodiment 1, wherein the heavy chain variable region comprises SEQ ID NO: 130. (Embodiment 90) The anti-TL1A antibody of embodiment 1, wherein the heavy chain variable region comprises a sequence at least about 90%, 95%, 96%, 97%, 98%, 99%, or 100% identical to SEQ ID NO: 130. (Embodiment 91) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 130. (Embodiment 92) The anti-TL1A antibody of embodiment 1, wherein the heavy chain variable region comprises SEQ ID NO: 131. (Embodiment 93) The anti-TL1A antibody of embodiment 1, wherein the heavy chain variable region comprises a sequence at least about 90%, 95%, 96%, 97%, 98%, 99%, or 100% identical to SEQ ID NO: 131. (Embodiment 94) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 131. (Embodiment 95) The anti-TL1A antibody of embodiment 1, wherein the heavy chain variable region comprises SEQ ID NO: 132. (Embodiment 96) The anti-TL1A antibody of embodiment 1, wherein the heavy chain variable region comprises a sequence at least about 90%, 95%, 96%, 97%, 98%, 99%, or 100% identical to SEQ ID NO: 132. (Embodiment 97) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 132.

(Embodiment 98) The anti-TL1A antibody of embodiment 1, wherein the heavy chain variable region comprises SEQ ID NO: 133. (Embodiment 99) The anti-TL1A antibody of embodiment 1, wherein the heavy chain variable region comprises a sequence at least about 90%, 95%, 96%, 97%, 98%, 99%, or 100% identical to SEQ ID NO: 133. (Embodiment 100) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 133. (Embodiment 101) The anti-TL1A antibody of embodiment 1, wherein the heavy chain variable region comprises SEQ ID NO: 134. (Embodiment 102) The anti-TL1A antibody of embodiment 1, wherein the heavy chain variable region comprises a sequence at least about 90%, 95%, 96%, 97%, 98%, 99%, or 100% identical to SEQ ID NO: 134. (Embodiment 103) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 134. (Embodiment 104) The anti-TL1A antibody of embodiment 1, wherein the heavy chain variable region comprises SEQ ID NO: 135. (Embodiment 105) The anti-TL1A antibody of embodiment 1, wherein the heavy chain variable region comprises a sequence at least about 90%, 95%, 96%, 97%, 98%, 99%, or 100% identical to SEQ ID NO: 135. (Embodiment 106) The anti-TL1A antibody of embodiment 1, wherein the heavy chain variable region comprises a sequence having about 1, 2, 3, 4, 5, 6, 7, 8, 9 or 10 amino acid substitutions or deletions as compared to SEQ ID NO: 135.

(Embodiment 107) The anti-TL1A antibody of any one of embodiments 1-106, wherein the light chain variable region comprises SEQ ID NO: 201. (Embodiment 108) The anti-TL1A antibody of any one of embodiments 1-106, wherein the light chain variable region comprises a sequence at least about 90%, 95%, 96%, 97%, 98%, 99%, or 100% identical to SEQ ID NO: 201. (Embodiment 109) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 201. (Embodiment 110) The anti-TL1A antibody of any one of embodiments 1-106, wherein the light chain variable region comprises SEQ ID NO: 202. (Embodiment 111) The anti-TL1A antibody of any one of embodiments 1-106, wherein the light chain variable region comprises a sequence at least about 90%, 95%, 96%, 97%, 98%, 99%, or 100% identical to SEQ ID NO: 202. (Embodiment 112) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 202. (Embodiment 113) The anti-TL1A antibody of any one of embodiments 1-106, wherein the light chain variable region comprises SEQ ID NO: 203. (Embodiment 114) The anti-TL1A antibody of any one of embodiments 1-106, wherein the light chain variable region comprises a sequence at least about 90%, 95%, 96%, 97%, 98%, 99%, or 100% identical to SEQ ID NO: 203. (Embodiment 115) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 203. (Embodiment 116) The anti-TL1A antibody of any one of embodiments 1-106, wherein the light chain variable region comprises SEQ ID NO: 204. (Embodiment 117) The anti-TL1A antibody of any one of embodiments 1-106, wherein the light chain variable region comprises a sequence at least about 90%, 95%, 96%, 97%, 98%, 99%, or 100% identical to SEQ ID NO: 204. (Embodiment 118) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 204. (Embodiment 119) The anti-TL1A antibody of any one of embodiments 1-106, wherein the light chain variable region comprises SEQ ID NO: 205. (Embodiment 120) The anti-TL1A antibody of any one of embodiments 1-106, wherein the light chain variable region comprises a sequence at least about 90%, 95%, 96%, 97%, 98%, 99%, or 100% identical to SEQ ID NO: 205. (Embodiment 121) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 205. (Embodiment 122) The anti-TL1A antibody of any one of embodiments 1-106, wherein the light chain variable region comprises SEQ ID NO: 206. (Embodiment 123) The anti-TL1A antibody of any one of embodiments 1-106, wherein the light chain variable region comprises a sequence at least about 90%, 95%, 96%, 97%, 98%, 99%, or 100% identical to SEQ ID NO: 206. (Embodiment 124) The anti-TL1A antibody of any one of embodiments 1-106, wherein the light chain variable region comprises a sequence having about 1, 2, 3, 4, 5, 6, 7, 8, 9 or 10 amino acid substitutions or deletions as compared to SEQ ID NO: 206.

(Embodiment 125) The anti-TL1A antibody of embodiment 1, comprising A217. (Embodiment 126) The anti-TL1A antibody of embodiment 1, wherein the heavy chain variable region comprises a sequence at least about 90%, 95%, 96%, 97%, 98%, 99%, or 100% identical to SEQ ID NO: 101, and the light chain variable region comprises a sequence at least about 90%, 95%, 96%, 97%, 98%, 99%, or 100% identical to SEQ ID NO: 201. (Embodiment 127) The anti-TL1A antibody of embodiment 1, comprising A220. (Embodiment 128) The anti-TL1A antibody of embodiment 1, wherein the heavy chain variable region comprises a sequence at least about 90%, 95%, 96%, 97%, 98%, 99%, or 100% identical to SEQ ID NO: 102, and the light chain variable region comprises a sequence at least about 90%, 95%, 96%, 97%, 98%, 99%, or 100% identical to SEQ ID NO: 201. (Embodiment 129) The anti-TL1A antibody of embodiment 1, comprising A223. (Embodiment 130) The anti-TL1A antibody of embodiment 1, wherein the heavy chain variable region comprises a sequence at least about 90%, 95%, 96%, 97%, 98%, 99%, or 100% identical to SEQ ID NO: 103, and the light chain variable region comprises a sequence at least about 90%, 95%, 96%, 97%, 98%, 99%, or 100% identical to SEQ ID NO: 202. (Embodiment 131) The anti-TL1A antibody of embodiment 1, comprising A219. (Embodiment 132) The anti-TL1A antibody of embodiment 1, wherein the heavy chain variable region comprises a sequence at least about 90%, 95%, 96%, 97%, 98%, 99%, or 100% identical to SEQ ID NO: 104, and the light chain variable region comprises a sequence at least about 90%, 95%, 96%, 97%, 98%, 99%, or 100% identical to SEQ ID NO: 201. (Embodiment 133) The anti-TL1A antibody of embodiment 1, comprising A221. (Embodiment 134) The anti-TL1A antibody of embodiment 1, wherein the heavy chain variable region comprises a sequence at least about 90%, 95%, 96%, 97%, 98%, 99%, or 100% identical to SEQ ID NO: 105, and the light chain variable region comprises a sequence at least about 90%, 95%, 96%, 97%, 98%, 99%, or 100% identical to SEQ ID NO: 201.

(Embodiment 135) The anti-TL1A antibody of embodiment 1, comprising A200. (Embodiment 136) The anti-TL1A antibody of embodiment 1, wherein the heavy chain variable region comprises a sequence at least about 90%, 95%, 96%, 97%, 98%, 99%, or 100% identical to SEQ ID NO: 103, and the light chain variable region comprises a sequence at least about 90%, 95%, 96%, 97%, 98%, 99%, or 100% identical to SEQ ID NO: 201. (Embodiment 137) The anti-TL1A antibody of embodiment 1, comprising A213. (Embodiment 138) The anti-TL1A antibody of embodiment 1, wherein the heavy chain variable region comprises a sequence at least about 90%, 95%, 96%, 97%, 98%, 99%, or 100% identical to SEQ ID NO: 106, and the light chain variable region comprises a sequence at least about 90%, 95%, 96%, 97%, 98%, 99%, or 100% identical to SEQ ID NO: 201. (Embodiment 139) The anti-TL1A antibody of embodiment 1, comprising A212. (Embodiment 140) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 202. (Embodiment 141) The anti-TL1A antibody of embodiment 1, comprising A107. (Embodiment 142) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 202. (Embodiment 143) The anti-TL1A antibody of embodiment 1, comprising A205. (Embodiment 144) The anti-TL1A antibody of embodiment 1, wherein the heavy chain variable region comprises a sequence at least about 90%, 95%, 96%, 97%, 98%, 99%, or 100% identical to SEQ ID NO: 109, and the light chain variable region comprises a sequence at least about 90%, 95%, 96%, 97%, 98%, 99%, or 100% identical to SEQ ID NO: 202.

(Embodiment 145) The anti-TL1A antibody of embodiment 1, comprising A211. (Embodiment 146) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 201. (Embodiment 147) The anti-TL1A antibody of embodiment 1, comprising A199. (Embodiment 148) The anti-TL1A antibody of embodiment 1, wherein the heavy chain variable region comprises a sequence at least about 90%, 95%, 96%, 97%, 98%, 99%, or 100% identical to SEQ ID NO: 109, and the light chain variable region comprises a sequence at least about 90%, 95%, 96%, 97%, 98%, 99%, or 100% identical to SEQ ID NO: 201. (Embodiment 149) The anti-TL1A antibody of embodiment 1, comprising A15. (Embodiment 150) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 203. (Embodiment 151) The anti-TL1A antibody of embodiment 1, comprising A30. (Embodiment 152) The anti-TL1A antibody of embodiment 1, wherein the heavy chain variable region comprises a sequence at least about 90%, 95%, 96%, 97%, 98%, 99%, or 100% identical to SEQ ID NO: 108, and the light chain variable region comprises a sequence at least about 90%, 95%, 96%, 97%, 98%, 99%, or 100% identical to SEQ ID NO: 204. (Embodiment 153) The anti-TL1A antibody of embodiment 1, comprising A100. (Embodiment 154) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 204.

(Embodiment 155) The anti-TL1A antibody of embodiment 1, comprising A181. (Embodiment 156) The anti-TL1A antibody of embodiment 1, wherein the heavy chain variable region comprises a sequence at least about 90%, 95%, 96%, 97%, 98%, 99%, or 100% identical to SEQ ID NO: 107, and the light chain variable region comprises a sequence at least about 90%, 95%, 96%, 97%, 98%, 99%, or 100% identical to SEQ ID NO: 202. (Embodiment 157) The anti-TL1A antibody of embodiment 1, comprising A129. (Embodiment 158) The anti-TL1A antibody of embodiment 1, wherein the heavy chain variable region comprises a sequence at least about 90%, 95%, 96%, 97%, 98%, 99%, or 100% identical to SEQ ID NO: 110, and the light chain variable region comprises a sequence at least about 90%, 95%, 96%, 97%, 98%, 99%, or 100% identical to SEQ ID NO: 204. (Embodiment 159) The anti-TL1A antibody of embodiment 1, comprising A214. (Embodiment 160) The anti-TL1A antibody of embodiment 1, wherein the heavy chain variable region comprises a sequence at least about 90%, 95%, 96%, 97%, 98%, 99%, or 100% identical to SEQ ID NO: 111, and the light chain variable region comprises a sequence at least about 90%, 95%, 96%, 97%, 98%, 99%, or 100% identical to SEQ ID NO: 201. (Embodiment 161) The anti-TL1A antibody of embodiment 1, comprising A216. (Embodiment 162) The anti-TL1A antibody of embodiment 1, wherein the heavy chain variable region comprises a sequence at least about 90%, 95%, 96%, 97%, 98%, 99%, or 100% identical to SEQ ID NO: 112, and the light chain variable region comprises a sequence at least about 90%, 95%, 96%, 97%, 98%, 99%, or 100% identical to SEQ ID NO: 201. (Embodiment 163) The anti-TL1A antibody of embodiment 1, comprising A122. (Embodiment 164) The anti-TL1A antibody of embodiment 1, wherein the heavy chain variable region comprises a sequence at least about 90%, 95%, 96%, 97%, 98%, 99%, or 100% identical to SEQ ID NO: 113, and the light chain variable region comprises a sequence at least about 90%, 95%, 96%, 97%, 98%, 99%, or 100% identical to SEQ ID NO: 204.

(Embodiment 165) The anti-TL1A antibody of embodiment 1, comprising A222. (Embodiment 166) The anti-TL1A antibody of embodiment 1, wherein the heavy chain variable region comprises a sequence at least about 90%, 95%, 96%, 97%, 98%, 99%, or 100% identical to SEQ ID NO: 114, and the light chain variable region comprises a sequence at least about 90%, 95%, 96%, 97%, 98%, 99%, or 100% identical to SEQ ID NO: 201. (Embodiment 167) The anti-TL1A antibody of embodiment 1, comprising A188. (Embodiment 168) The anti-TL1A antibody of embodiment 1, wherein the heavy chain variable region comprises a sequence at least about 90%, 95%, 96%, 97%, 98%, 99%, or 100% identical to SEQ ID NO: 115, and the light chain variable region comprises a sequence at least about 90%, 95%, 96%, 97%, 98%, 99%, or 100% identical to SEQ ID NO: 202. (Embodiment 169) The anti-TL1A antibody of embodiment 1, comprising A203. (Embodiment 170) The anti-TL1A antibody of embodiment 1, wherein the heavy chain variable region comprises a sequence at least about 90%, 95%, 96%, 97%, 98%, 99%, or 100% identical to SEQ ID NO: 116, and the light chain variable region comprises a sequence at least about 90%, 95%, 96%, 97%, 98%, 99%, or 100% identical to SEQ ID NO: 201. (Embodiment 171) The anti-TL1A antibody of embodiment 1, comprising A147. (Embodiment 172) The anti-TL1A antibody of embodiment 1, wherein the heavy chain variable region comprises a sequence at least about 90%, 95%, 96%, 97%, 98%, 99%, or 100% identical to SEQ ID NO: 117, and the light chain variable region comprises a sequence at least about 90%, 95%, 96%, 97%, 98%, 99%, or 100% identical to SEQ ID NO: 201. (Embodiment 173) The anti-TL1A antibody of embodiment 1, comprising A127. (Embodiment 174) The anti-TL1A antibody of embodiment 1, wherein the heavy chain variable region comprises a sequence at least about 90%, 95%, 96%, 97%, 98%, 99%, or 100% identical to SEQ ID NO: 118, and the light chain variable region comprises a sequence at least about 90%, 95%, 96%, 97%, 98%, 99%, or 100% identical to SEQ ID NO: 204.

(Embodiment 175) The anti-TL1A antibody of embodiment 1, comprising A126. (Embodiment 176) The anti-TL1A antibody of embodiment 1, wherein the heavy chain variable region comprises a sequence at least about 90%, 95%, 96%, 97%, 98%, 99%, or 100% identical to SEQ ID NO: 114, and the light chain variable region comprises a sequence at least about 90%, 95%, 96%, 97%, 98%, 99%, or 100% identical to SEQ ID NO: 204. (Embodiment 177) The anti-TL1A antibody of embodiment 1, comprising A160. (Embodiment 178) The anti-TL1A antibody of embodiment 1, wherein the heavy chain variable region comprises a sequence at least about 90%, 95%, 96%, 97%, 98%, 99%, or 100% identical to SEQ ID NO: 102, and the light chain variable region comprises a sequence at least about 90%, 95%, 96%, 97%, 98%, 99%, or 100% identical to SEQ ID NO: 204. (Embodiment 179) The anti-TL1A antibody of embodiment 1, comprising A157. (Embodiment 180) The anti-TL1A antibody of embodiment 1, wherein the heavy chain variable region comprises a sequence at least about 90%, 95%, 96%, 97%, 98%, 99%, or 100% identical to SEQ ID NO: 104, and the light chain variable region comprises a sequence at least about 90%, 95%, 96%, 97%, 98%, 99%, or 100% identical to SEQ ID NO: 204. (Embodiment 181) The anti-TL1A antibody of embodiment 1, comprising A159. (Embodiment 182) The anti-TL1A antibody of embodiment 1, wherein the heavy chain variable region comprises a sequence at least about 90%, 95%, 96%, 97%, 98%, 99%, or 100% identical to SEQ ID NO: 119, and the light chain variable region comprises a sequence at least about 90%, 95%, 96%, 97%, 98%, 99%, or 100% identical to SEQ ID NO: 204. (Embodiment 183) The anti-TL1A antibody of embodiment 1, comprising A218. (Embodiment 184) The anti-TL1A antibody of embodiment 1, wherein the heavy chain variable region comprises a sequence at least about 90%, 95%, 96%, 97%, 98%, 99%, or 100% identical to SEQ ID NO: 119, and the light chain variable region comprises a sequence at least about 90%, 95%, 96%, 97%, 98%, 99%, or 100% identical to SEQ ID NO: 201.

(Embodiment 185) The anti-TL1A antibody of embodiment 1, comprising A158. (Embodiment 186) The anti-TL1A antibody of embodiment 1, wherein the heavy chain variable region comprises a sequence at least about 90%, 95%, 96%, 97%, 98%, 99%, or 100% identical to SEQ ID NO: 101, and the light chain variable region comprises a sequence at least about 90%, 95%, 96%, 97%, 98%, 99%, or 100% identical to SEQ ID NO: 204. (Embodiment 187) The anti-TL1A antibody of embodiment 1, comprising A125. (Embodiment 188) The anti-TL1A antibody of embodiment 1, wherein the heavy chain variable region comprises a sequence at least about 90%, 95%, 96%, 97%, 98%, 99%, or 100% identical to SEQ ID NO: 105, and the light chain variable region comprises a sequence at least about 90%, 95%, 96%, 97%, 98%, 99%, or 100% identical to SEQ ID NO: 204. (Embodiment 189) The anti-TL1A antibody of embodiment 1, comprising A103. (Embodiment 190) The anti-TL1A antibody of embodiment 1, wherein the heavy chain variable region comprises a sequence at least about 90%, 95%, 96%, 97%, 98%, 99%, or 100% identical to SEQ ID NO: 120, and the light chain variable region comprises a sequence at least about 90%, 95%, 96%, 97%, 98%, 99%, or 100% identical to SEQ ID NO: 204. (Embodiment 191) The anti-TL1A antibody of embodiment 1, comprising A64. (Embodiment 192) The anti-TL1A antibody of embodiment 1, wherein the heavy chain variable region comprises a sequence at least about 90%, 95%, 96%, 97%, 98%, 99%, or 100% identical to SEQ ID NO: 121, and the light chain variable region comprises a sequence at least about 90%, 95%, 96%, 97%, 98%, 99%, or 100% identical to SEQ ID NO: 202. (Embodiment 193) The anti-TL1A antibody of embodiment 1, comprising A67. (Embodiment 194) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 202.

(Embodiment 195) The anti-TL1A antibody of embodiment 1, comprising A138. (Embodiment 196) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 204. (Embodiment 197) The anti-TL1A antibody of embodiment 1, comprising A68. (Embodiment 198) The anti-TL1A antibody of embodiment 1, wherein the heavy chain variable region comprises a sequence at least about 90%, 95%, 96%, 97%, 98%, 99%, or 100% identical to SEQ ID NO: 123, and the light chain variable region comprises a sequence at least about 90%, 95%, 96%, 97%, 98%, 99%, or 100% identical to SEQ ID NO: 202. (Embodiment 199) The anti-TL1A antibody of embodiment 1, comprising A94. (Embodiment 200) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable region comprises a sequence at least about 90%, 95%, 96%, 97%, 98%, 99%, or 100% identical to SEQ ID NO: 202. (Embodiment 201) The anti-TL1A antibody of embodiment 1, comprising A110. (Embodiment 202) The anti-TL1A antibody of embodiment 1, wherein the heavy chain variable region comprises a sequence at least about 90%, 95%, 96%, 97%, 98%, 99%, or 100% identical to SEQ ID NO: 125, and the light chain variable region comprises a sequence at least about 90%, 95%, 96%, 97%, 98%, 99%, or 100% identical to SEQ ID NO: 205. (Embodiment 203) The anti-TL1A antibody of embodiment 1, comprising A197. (Embodiment 204) The anti-TL1A antibody of embodiment 1, wherein the heavy chain variable region comprises a sequence at least about 90%, 95%, 96%, 97%, 98%, 99%, or 100% identical to SEQ ID NO: 116, and the light chain variable region comprises a sequence at least about 90%, 95%, 96%, 97%, 98%, 99%, or 100% identical to SEQ ID NO: 205.

(Embodiment 205) The anti-TL1A antibody of embodiment 1, comprising A112. (Embodiment 206) The anti-TL1A antibody of embodiment 1, wherein the heavy chain variable region comprises a sequence at least about 90%, 95%, 96%, 97%, 98%, 99%, or 100% identical to SEQ ID NO: 117, and the light chain variable region comprises a sequence at least about 90%, 95%, 96%, 97%, 98%, 99%, or 100% identical to SEQ ID NO: 205. (Embodiment 207) The anti-TL1A antibody of embodiment 1, comprising A169. (Embodiment 208) The anti-TL1A antibody of embodiment 1, wherein the heavy chain variable region comprises a sequence at least about 90%, 95%, 96%, 97%, 98%, 99%, or 100% identical to SEQ ID NO: 126, and the light chain variable region comprises a sequence at least about 90%, 95%, 96%, 97%, 98%, 99%, or 100% identical to SEQ ID NO: 205. (Embodiment 209) The anti-TL1A antibody of embodiment 1, comprising A173. (Embodiment 210) The anti-TL1A antibody of embodiment 1, wherein the heavy chain variable region comprises a sequence at least about 90%, 95%, 96%, 97%, 98%, 99%, or 100% identical to SEQ ID NO: 127, and the light chain variable region comprises a sequence at least about 90%, 95%, 96%, 97%, 98%, 99%, or 100% identical to SEQ ID NO: 205. (Embodiment 211) The anti-TL1A antibody of embodiment 1, comprising A179. (Embodiment 212) The anti-TL1A antibody of embodiment 1, wherein the heavy chain variable region comprises a sequence at least about 90%, 95%, 96%, 97%, 98%, 99%, or 100% identical to SEQ ID NO: 127, and the light chain variable region comprises a sequence at least about 90%, 95%, 96%, 97%, 98%, 99%, or 100% identical to SEQ ID NO: 201. (Embodiment 213) The anti-TL1A antibody of embodiment 1, comprising A148. (Embodiment 214) The anti-TL1A antibody of embodiment 1, wherein the heavy chain variable region comprises a sequence at least about 90%, 95%, 96%, 97%, 98%, 99%, or 100% identical to SEQ ID NO: 121, and the light chain variable region comprises a sequence at least about 90%, 95%, 96%, 97%, 98%, 99%, or 100% identical to SEQ ID NO: 201.

(Embodiment 215) The anti-TL1A antibody of embodiment 1, comprising A115. (Embodiment 216) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 205. (Embodiment 217) The anti-TL1A antibody of embodiment 1, comprising A149. (Embodiment 218) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 201. (Embodiment 219) The anti-TL1A antibody of embodiment 1, comprising A134. (Embodiment 220) The anti-TL1A antibody of embodiment 1, wherein the heavy chain variable region comprises a sequence at least about 90%, 95%, 96%, 97%, 98%, 99%, or 100% identical to SEQ ID NO: 122, and the light chain variable region comprises a sequence at least about 90%, 95%, 96%, 97%, 98%, 99%, or 100% identical to SEQ ID NO: 206. (Embodiment 221) The anti-TL1A antibody of embodiment 1, comprising A113. (Embodiment 222) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable region comprises a sequence at least about 90%, 95%, 96%, 97%, 98%, 99%, or 100% identical to SEQ ID NO: 205. (Embodiment 223) The anti-TL1A antibody of embodiment 1, comprising A151. (Embodiment 224) The anti-TL1A antibody of embodiment 1, wherein the heavy chain variable region comprises a sequence at least about 90%, 95%, 96%, 97%, 98%, 99%, or 100% identical to SEQ ID NO: 124, and the light chain variable region comprises a sequence at least about 90%, 95%, 96%, 97%, 98%, 99%, or 100% identical to SEQ ID NO: 201.

(Embodiment 225) The anti-TL1A antibody of embodiment 1, comprising A96. (Embodiment 226) The anti-TL1A antibody of embodiment 1, wherein the heavy chain variable region comprises a sequence at least about 90%, 95%, 96%, 97%, 98%, 99%, or 100% identical to SEQ ID NO: 128, and the light chain variable region comprises a sequence at least about 90%, 95%, 96%, 97%, 98%, 99%, or 100% identical to SEQ ID NO: 205. (Embodiment 227) The anti-TL1A antibody of embodiment 1, comprising A132. (Embodiment 228) The anti-TL1A antibody of embodiment 1, wherein the heavy chain variable region comprises a sequence at least about 90%, 95%, 96%, 97%, 98%, 99%, or 100% identical to SEQ ID NO: 128, and the light chain variable region comprises a sequence at least about 90%, 95%, 96%, 97%, 98%, 99%, or 100% identical to SEQ ID NO: 206. (Embodiment 229) The anti-TL1A antibody of embodiment 1, comprising A196. (Embodiment 230) The anti-TL1A antibody of embodiment 1, wherein the heavy chain variable region comprises a sequence at least about 90%, 95%, 96%, 97%, 98%, 99%, or 100% identical to SEQ ID NO: 129, and the light chain variable region comprises a sequence at least about 90%, 95%, 96%, 97%, 98%, 99%, or 100% identical to SEQ ID NO: 205. (Embodiment 231) The anti-TL1A antibody of embodiment 1, comprising A172. (Embodiment 232) The anti-TL1A antibody of embodiment 1, wherein the heavy chain variable region comprises a sequence at least about 90%, 95%, 96%, 97%, 98%, 99%, or 100% identical to SEQ ID NO: 130, and the light chain variable region comprises a sequence at least about 90%, 95%, 96%, 97%, 98%, 99%, or 100% identical to SEQ ID NO: 205. (Embodiment 233) The anti-TL1A antibody of embodiment 1, comprising A75. (Embodiment 234) The anti-TL1A antibody of embodiment 1, wherein the heavy chain variable region comprises a sequence at least about 90%, 95%, 96%, 97%, 98%, 99%, or 100% identical to SEQ ID NO: 131, and the light chain variable region comprises a sequence at least about 90%, 95%, 96%, 97%, 98%, 99%, or 100% identical to SEQ ID NO: 205.

(Embodiment 235) The anti-TL1A antibody of embodiment 1, comprising A174. (Embodiment 236) The anti-TL1A antibody of embodiment 1, wherein the heavy chain variable region comprises a sequence at least about 90%, 95%, 96%, 97%, 98%, 99%, or 100% identical to SEQ ID NO: 132, and the light chain variable region comprises a sequence at least about 90%, 95%, 96%, 97%, 98%, 99%, or 100% identical to SEQ ID NO: 205. (Embodiment 237) The anti-TL1A antibody of embodiment 1, comprising A109. (Embodiment 238) The anti-TL1A antibody of embodiment 1, wherein the heavy chain variable region comprises a sequence at least about 90%, 95%, 96%, 97%, 98%, 99%, or 100% identical to SEQ ID NO: 133, and the light chain variable region comprises a sequence at least about 90%, 95%, 96%, 97%, 98%, 99%, or 100% identical to SEQ ID NO: 205. (Embodiment 239) The anti-TL1A antibody of embodiment 1, comprising A198. (Embodiment 240) The anti-TL1A antibody of embodiment 1, wherein the heavy chain variable region comprises a sequence at least about 90%, 95%, 96%, 97%, 98%, 99%, or 100% identical to SEQ ID NO: 134, and the light chain variable region comprises a sequence at least about 90%, 95%, 96%, 97%, 98%, 99%, or 100% identical to SEQ ID NO: 205. (Embodiment 241) The anti-TL1A antibody of embodiment 1, comprising A170. (Embodiment 242) The anti-TL1A antibody of embodiment 1, wherein the heavy chain variable region comprises a sequence at least about 90%, 95%, 96%, 97%, 98%, 99%, or 100% identical to SEQ ID NO: 135, and the light chain variable region comprises a sequence at least about 90%, 95%, 96%, 97%, 98%, 99%, or 100% identical to SEQ ID NO: 205. (Embodiment 243) The anti-TL1A antibody of embodiment 1, comprising A500. (Embodiment 244) The anti-TL1A antibody of embodiment 1, comprising A501.

(Embodiment 245) The anti-TL1A of any one of embodiments 1-244, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. (Embodiment 246) The anti-TL1A of any one of embodiments 1-244, comprising a (i) human IgG4 Fc region or (ii) a human IgG4 Fc region comprising (a) S228P and L235E, or (b) S228P, F234A, and L235A, per Kabat numbering. (Embodiment 247) The anti-TL1A of any one of embodiments 1-244, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). (Embodiment 248) The anti-TL1A of any one of embodiments 1-247, comprising a heavy chain Fc region comprising any one of SEQ ID NOS: 320-362. (Embodiment 249) The anti-TL1A of any one of embodiments 1-248, comprising a light chain constant region comprising SEQ ID NO: 319.

(Embodiment 250) The anti-TL1A of any one of embodiments 1-249, comprising at least about 80% monomeric fraction as determined by the size exclusion chromatography method described herein. (Embodiment 251) The anti-TL1A of any one of embodiments 1-250, comprising at least about 81%, at least about 82%, at least about 83%, or at least about 84% monomeric fraction as determined by the size exclusion chromatography method described herein. (Embodiment 252) The anti-TL1A of any one of embodiments 1-251, comprising at least about 85% monomeric fraction as determined by the size exclusion chromatography method described herein. (Embodiment 253) The anti-TL1A of any one of embodiments 1-252, comprising at least about 86%, at least about 87%, at least about 88%, or at least about 89% monomeric fraction as determined by the size exclusion chromatography method described herein. (Embodiment 254) The anti-TL1A of any one of embodiments 1-253, comprising at least about 90% monomeric fraction as determined by the size exclusion chromatography method described herein. (Embodiment 255) The anti-TL1A of any one of embodiments 1-254, comprising at least about 91%, at least about 92%, at least about 93%, or at least about 94% monomeric fraction as determined by the size exclusion chromatography method described herein. (Embodiment 256) The anti-TL1A of any one of embodiments 1-255, comprising at least about 95% monomeric fraction as determined by the size exclusion chromatography method described herein. (Embodiment 257) The anti-TL1A of any one of embodiments 1-256, comprising at least about 96%, at least about 97%, at least about 98%, or at least about 99% monomeric fraction as determined by the size exclusion chromatography method described herein.

(Embodiment 258) The anti-TL1A of any one of embodiments 1-257, comprising at least about 2 μg/mL expression as determined by the method disclosed herein. (Embodiment 259) The anti-TL1A of any one of embodiments 1-258, comprising between about 2 μg/mL and about 60 μg/mL expression as determined by the method disclosed herein. (Embodiment 260) The anti-TL1A of any one of embodiments 1-259, comprising between about 5 μg/mL and about 60 μg/mL expression as determined by the method disclosed herein. (Embodiment 261) The anti-TL1A of any one of embodiments 1-260, comprising between about 10 μg/mL and about 60 μg/mL expression as determined by the method disclosed herein. (Embodiment 262) The anti-TL1A of any one of embodiments 1-261, comprising at least about 5 μg/mL expression as determined by the method disclosed herein. (Embodiment 263) The anti-TL1A of any one of embodiments 1-262, comprising at least about 10 μg/mL expression as determined by the method disclosed herein. (Embodiment 264) The anti-TL1A of any one of embodiments 1-263, comprising at least about 15 μg/mL expression as determined by the method disclosed herein. (Embodiment 265) The anti-TL1A of any one of embodiments 1-264, comprising at least about 20 μg/mL expression as determined by the method disclosed herein.

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1200-1263; and a light chain variable region at least about 85%, 90%, 91%, 92%, 93%, 94%, 95%, 96%, 97%, 98%, 99%, or 100% identical to any one of SEQ ID NOS: 1264-1300, 1304-1316.

In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 136; and a light chain variable region at least about 85%, 90%, 91%, 92%, 93%, 94%, 95%, 96%, 97%, 98%, 99%, or 100% identical to SEQ ID NO: 202 (clone 34). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1200; and a light chain variable region at least about 85%, 90%, 91%, 92%, 93%, 94%, 95%, 96%, 97%, 98%, 99%, or 100% identical to SEQ ID NO: 12646 (5C3D11). In one aspect, provided is an anti-TL1A antibody comprising a heavy chain variable region comprising an amino acid sequence at least about 85%, 90%, 91%, 92%, 93%, 94%, 95%, 96%, 97% 98% 99%, or 100% identical to SEQ ID NO: 1201; and a light chain variable region at least about 85%, 90%, 91%, 92%, 93%, 94%, 95%, 96%, 97%, 98%, 99%, or 100% identical to SEQ ID NO: 1265 (9E12E5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1202; and a light chain variable region at least about 85%, 90%, 91%, 92%, 93%, 94%, 95%, 96%, 97%, 98%, 99%, or 100% identical to SEQ ID NO: 208 (AS12824). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1203; and a light chain variable region at least about 85%, 90%, 91%, 92%, 93%, 94%, 95%, 96%, 97%, 98%, 99%, or 100% identical to SEQ ID NO: 1266 (AS12823). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1204; and a light chain variable region at least about 85%, 90%, 91%, 92%, 93%, 94%, 95%, 96%, 97%, 98%, 99%, or 100% identical to SEQ ID NO: 1267 (AS12819). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1205; and a light chain variable region at least about 85%, 90%, 91%, 92%, 93%, 94%, 95%, 96%, 97%, 98%, 99%, or 100% identical to SEQ ID NO: 1268 (AS12816). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1206; and a light chain variable region at least about 85%, 90%, 91%, 92%, 93%, 94%, 95%, 96%, 97%, 98%, 99%, or 100% identical to SEQ ID NO: 1269 (AS12825). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1207; and a light chain variable region at least about 85%, 90%, 91%, 92%, 93%, 94%, 95%, 96%, 97%, 98%, 99%, or 100% identical to SEQ ID NO: 1270 (12835). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 202 (18-7 S93E). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 203 (18-7). In one aspect, provided is an anti-TL1A antibody comprising a heavy chain variable region comprising an amino acid sequence at least about 85%, 90%, 91%, 92% 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1272 (18-7 S92D). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1273 (18-7 S92H). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1274 (18-7 S92N). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1275 (18-7 S92Q). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1208; and a light chain variable region at least about 85%, 90%, 91%, 92%, 93%, 94%, 95%, 96%, 97%, 98%, 99%, or 100% identical to SEQ ID NO: 1276 (18-7 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1209; and a light chain variable region at least about 85%, 90%, 91%, 92%, 93%, 94%, 95%, 96%, 97%, 98%, 99%, or 100% identical to SEQ ID NO: 1271 (21-3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1210; and a light chain variable region at least about 85%, 90%, 91%, 92%, 93%, 94%, 95%, 96%, 97%, 98%, 99%, or 100% identical to SEQ ID NO: 1271 (21-3 V102K). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1211; and a light chain variable region at least about 85%, 90%, 91%, 92%, 93%, 94%, 95%, 96%, 97%, 98%, 99%, or 100% identical to SEQ ID NO: 1271 (21-3 V102M). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1212; and a light chain variable region at least about 85%, 90%, 91%, 92%, 93%, 94%, 95%, 96%, 97%, 98%, 99%, or 100% identical to SEQ ID NO: 1271 (21-3 V102Q). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1213; and a light chain variable region at least about 85%, 90%, 91%, 92%, 93%, 94%, 95%, 96%, 97%, 98%, 99%, or 100% identical to SEQ ID NO: 1271 (21-3 V102 W). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1214; and a light chain variable region at least about 85%, 90%, 91%, 92%, 93%, 94%, 95%, 96%, 97%, 98%, 99%, or 100% identical to SEQ ID NO: 1271 (21-3 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1215; and a light chain variable region at least about 85%, 90%, 91%, 92%, 93%, 94%, 95%, 96%, 97%, 98%, 99%, or 100% identical to SEQ ID NO: 1271 (21-3 CDRv). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1216; and a light chain variable region at least about 85%, 90%, 91%, 92%, 93%, 94%, 95%, 96%, 97%, 98%, 99%, or 100% identical to SEQ ID NO: 202 (clone 2). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1216; and a light chain variable region at least about 85%, 90%, 91%, 92%, 93%, 94%, 95%, 96%, 97%, 98%, 99%, or 100% identical to SEQ ID NO: 1277 (clone 52). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1217; and a light chain variable region at least about 85%, 90%, 91%, 92%, 93%, 94%, 95%, 96%, 97%, 98%, 99%, or 100% identical to SEQ ID NO: 202 (clone 46). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1218; and a light chain variable region at least about 85%, 90%, 91%, 92%, 93%, 94%, 95%, 96%, 97%, 98%, 99%, or 100% identical to SEQ ID NO: 202 (clone 47). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1219; and a light chain variable region at least about 85%, 90%, 91%, 92%, 93%, 94%, 95%, 96%, 97%, 98%, 99%, or 100% identical to SEQ ID NO: 1275 (clone 14). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1220; and a light chain variable region at least about 85%, 90%, 91%, 92%, 93%, 94%, 95%, 96%, 97%, 98%, 99%, or 100% identical to SEQ ID NO: 1275 (clone 16L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1221; and a light chain variable region at least about 85%, 90%, 91%, 92%, 93%, 94%, 95%, 96%, 97%, 98%, 99%, or 100% identical to SEQ ID NO: 1275 (clone 17L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1222; and a light chain variable region at least about 85%, 90%, 91%, 92%, 93%, 94%, 95%, 96%, 97%, 98%, 99%, or 100% identical to SEQ ID NO: 1275 (clone 17L-1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1223; and a light chain variable region at least about 85%, 90%, 91%, 92%, 93%, 94%, 95%, 96%, 97%, 98%, 99%, or 100% identical to SEQ ID NO: 202 (clone 23). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1224; and a light chain variable region at least about 85%, 90%, 91%, 92%, 93%, 94%, 95%, 96%, 97%, 98%, 99%, or 100% identical to SEQ ID NO: 202 (clone 53). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1224; and a light chain variable region at least about 85%, 90%, 91%, 92%, 93%, 94%, 95%, 96%, 97%, 98%, 99%, or 100% identical to SEQ ID NO: 1277 (clone E1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1225; and a light chain variable region at least about 85%, 90%, 91%, 92%, 93%, 94%, 95%, 96%, 97%, 98%, 99%, or 100% identical to SEQ ID NO: 1275 (clone 3-17L V-A). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1226; and a light chain variable region at least about 85%, 90%, 91%, 92%, 93%, 94%, 95%, 96%, 97%, 98%, 99%, or 100% identical to SEQ ID NO: 1275 (clone 3-17L). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1227; and a light chain variable region at least about 85%, 90%, 91%, 92%, 93%, 94%, 95%, 96%, 97%, 98%, 99%, or 100% identical to SEQ ID NO: 203 (clone L8 mod). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1209; and a light chain variable region at least about 85%, 90%, 91%, 92%, 93%, 94%, 95%, 96%, 97%, 98%, 99%, or 100% identical to SEQ ID NO: 203 (clone L8). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1228; and a light chain variable region at least about 85%, 90%, 91%, 92%, 93%, 94%, 95%, 96%, 97%, 98%, 99%, or 100% identical to SEQ ID NO: 203 (clone X-V). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 203 (clone X). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 1278 (clone XL3-6). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 1279 (clone XL3-10). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 207 (clone XL3-15). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1229; and a light chain variable region at least about 85%, 90%, 91%, 92%, 93%, 94%, 95%, 96%, 97%, 98%, 99%, or 100% identical to SEQ ID NO: 204 (clone L3-13). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1230; and a light chain variable region at least about 85%, 90%, 91%, 92%, 93%, 94%, 95%, 96%, 97%, 98%, 99%, or 100% identical to SEQ ID NO: 203 (clone H3-1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1231; and a light chain variable region at least about 85%, 90%, 91%, 92%, 93%, 94%, 95%, 96%, 97% 98% 99%, or 100% identical to SEQ ID NO: 203 (clone H2-2). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1232; and a light chain variable region at least about 85%, 90%, 91%, 92%, 93%, 94%, 95%, 96%, 97%, 98%, 99%, or 100% identical to SEQ ID NO: 203 (clone H2-5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1233; and a light chain variable region at least about 85%, 90%, 91%, 92%, 93%, 94%, 95%, 96%, 97%, 98%, 99%, or 100% identical to SEQ ID NO: 1280 (M1). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1234; and a light chain variable region at least about 85%, 90%, 91%, 92%, 93%, 94%, 95%, 96%, 97%, 98%, 99%, or 100% identical to SEQ ID NO: 1281 (M2). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1235; and a light chain variable region at least about 85%, 90%, 91%, 92%, 93%, 94%, 95%, 96%, 97%, 98%, 99%, or 100% identical to SEQ ID NO: 1282 (M3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1235; and a light chain variable region at least about 85%, 90%, 91%, 92%, 93%, 94%, 95%, 96%, 97%, 98%, 99%, or 100% identical to SEQ ID NO: 1283 (M3). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1237; and a light chain variable region at least about 85%, 90%, 91%, 92%, 93%, 94%, 95%, 96%, 97%, 98%, 99%, or 100% identical to SEQ ID NO: 1284 (M4). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1238; and a light chain variable region at least about 85%, 90%, 91%, 92%, 93%, 94%, 95%, 96%, 97%, 98%, 99%, or 100% identical to SEQ ID NO: 1285 (M5). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1239-1242; and a light chain variable region at least about 85%, 90%, 91%, 92%, 93%, 94%, 95%, 96%, 97%, 98%, 99%, or 100% identical to any one of SEQ ID NOS: 1286-1293 (M6). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1243-1247; and a light chain variable region at least about 85%, 90%, 91%, 92%, 93%, 94%, 95%, 96%, 97%, 98%, 99%, or 100% identical to any one of SEQ ID NOS: 1294-1297 (M7). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1248-1259; and a light chain variable region at least about 85%, 90%, 91%, 92%, 93%, 94%, 95%, 96%, 97%, 98%, 99%, or 100% identical to any one of SEQ ID NOS: 1298-1312 (M8). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1260; and a light chain variable region at least about 85%, 90%, 91%, 92%, 93%, 94%, 95%, 96%, 97%, 98%, 99%, or 100% identical to SEQ ID NO: 1313 (M9). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1261; and a light chain variable region at least about 85%, 90%, 91%, 92%, 93%, 94%, 95%, 96%, 97%, 98%, 99%, or 100% identical to SEQ ID NO: 1314 (M10). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1262; and a light chain variable region at least about 85%, 90%, 91%, 92%, 93%, 94%, 95%, 96%, 97%, 98%, 99%, or 100% identical to SEQ ID NO: 1315 (M11). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1263; and a light chain variable region at least about 85%, 90%, 91%, 92%, 93%, 94%, 95%, 96%, 97%, 98%, 99%, or 100% identical to SEQ ID NO: 1316 (M12). In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 301; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 302; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1301; and a light chain variable region at least about 85%, 90%, 91%, 92%, 93%, 94%, 95%, 96%, 97%, 98%, 99%, or 100% identical to SEQ ID NO: 303. In one aspect, provided is an anti-TL1A antibody comprising a heavy chain variable region comprising an amino acid sequence at least about 85%, 90%, 91%, 92%, 93%, 94%, 95%, 96%, 97%, 98%, 99%, or 100% identical to SEQ ID NO: 1302; and a light chain variable region at least about 85%, 90%, 91%, 92%, 93%, 94%, 95%, 96%, 97%, 98%, 99%, or 100% identical to SEQ ID NO: 303.

### 5.4.1.4 Exemplary Anti-TL1A Constant Regions

In some embodiments, one or more amino acid modifications may be introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In an exemplary embodiment, the Fc region comprises any one of SEQ ID NOS: 320-362, 401-413, 501-515.

In some embodiments, antibodies of this disclosure have a reduced effector function as compared to a human IgG. Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

Some Fe regions have a natural lack of effector function, and some Fc regions can comprise mutations that reduce effector functions. For instance, IgG4 has low ADCC and CDC activities and IgG2 has low ADCC activity.

The disclosure provides antibodies comprising Fc regions characterized by exhibiting ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1, SEQ ID NO: 320). The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1, SEQ ID NO: 320). In certain embodiments, the antibodies of this disclosure have reduced effector function as compared with human IgG1. Measurement of effector function may be performed as described in Example 3.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In some embodiments, an antibody comprises an IgG1 Fc region comprising an N297A substitution, according to the Kabat numbering system. In some embodiments, an antibody comprises an IgG1 Fc region comprising an N297Q substitution, according to the Kabat numbering system. In some embodiments, an antibody comprises an IgG1 Fc region comprising an N297D substitution, according to the Kabat numbering system. In some embodiments, an antibody comprises an IgG1 Fc region comprising an D265A substitution, according to the Kabat numbering system. In some embodiments, an antibody comprises an IgG1 Fc region comprising an S228P substitution, according to the Kabat numbering system. In some embodiments, an antibody comprises an IgG1 Fc region comprising an L235A substitution, according to the Kabat numbering system. In some embodiments, an antibody comprises an IgG1 Fc region comprising an L237A substitution, according to the Kabat numbering system. In some embodiments, an antibody comprises an IgG1 Fc region comprising an L234A substitution, according to the Kabat numbering system. In some embodiments, an antibody comprises an IgG1 Fc region comprising an E233P substitution, according to the Kabat numbering system. In some embodiments, an antibody comprises an IgG1 Fc region comprising an L234V substitution, according to the Kabat numbering system. In some embodiments, an antibody comprises an IgG1 Fc region comprising an C236 deletion, according to the Kabat numbering system. In some embodiments, an antibody comprises an IgG1 Fc region comprising a P238A substitution, according to the Kabat numbering system. In some embodiments, an antibody comprises an IgG1 Fc region comprising an A327Q substitution, according to the Kabat numbering system. In some embodiments, an antibody comprises an IgG1 Fc region comprising a P329A substitution, according to the Kabat numbering system. In some embodiments, an antibody comprises an IgG1 Fc region comprising an P329G substitution, according to the Kabat numbering system. In some embodiments, an antibody comprises an IgG1 Fc region comprising an L235E substitution, according to the Kabat numbering system. In some embodiments, an antibody comprises an IgG1 Fc region comprising an P331S substitution, according to the Kabat numbering system. In some embodiments, an antibody comprises an IgG1 Fc region comprising an L234F substitution, according to the Kabat numbering system. In some embodiments, an antibody comprises an IgG1 Fe region comprising a 235G substitution, according to the Kabat numbering system. In some embodiments, an antibody comprises an IgG1 Fe region comprising an 235Q substitution, according to the Kabat numbering system. In some embodiments, an antibody comprises an IgG1 Fc region comprising an 235R substitution, according to the Kabat numbering system. In some embodiments, an antibody comprises an IgG1 Fc region comprising an 235S substitution, according to the Kabat numbering system. In some embodiments, an antibody comprises an IgG1 Fc region comprising an 236F substitution, according to the Kabat numbering system. In some embodiments, an antibody comprises an IgG1 Fc region comprising an 236R substitution, according to the Kabat numbering system. In some embodiments, an antibody comprises an IgG1 Fc region comprising an 237E substitution, according to the Kabat numbering system. In some embodiments, an antibody comprises an IgG1 Fc region comprising an 237K substitution, according to the Kabat numbering system. In some embodiments, an antibody comprises an IgG1 Fc region comprising an 237N substitution, according to the Kabat numbering system. In some embodiments, an antibody comprises an IgG1 Fe region comprising an 237R substitution, according to the Kabat numbering system. In some embodiments, an antibody comprises an IgG1 Fc region comprising an 238A substitution, according to the Kabat numbering system. In some embodiments, an antibody comprises an IgG1 Fc region comprising an 238E substitution, according to the Kabat numbering system. In some embodiments, an antibody comprises an IgG1 Fc region comprising an 238G substitution, according to the Kabat numbering system. In some embodiments, an antibody comprises an IgG1 Fc region comprising an 238H substitution, according to the Kabat numbering system. In some embodiments, an antibody comprises an IgG1 Fc region comprising an 238I substitution, according to the Kabat numbering system. In some embodiments, an antibody comprises an IgG1 Fc region comprising an 238V substitution, according to the Kabat numbering system. In some embodiments, an antibody comprises an IgG1 Fc region comprising an 238 W substitution, according to the Kabat numbering system. In some embodiments, an antibody comprises an IgG1 Fc region comprising an 238Y substitution, according to the Kabat numbering system. In some embodiments, an antibody comprises an IgG1 Fc region comprising an 248A substitution, according to the Kabat numbering system. In some embodiments, an antibody comprises an IgG1 Fc region comprising an 254D substitution, according to the Kabat numbering system. In some embodiments, an antibody comprises an IgG1 Fc region comprising an 254E substitution, according to the Kabat numbering system. In some embodiments, an antibody comprises an IgG1 Fc region comprising an 254G substitution, according to the Kabat numbering system. In some embodiments, an antibody comprises an IgG1 Fc region comprising an 254H substitution, according to the Kabat numbering system. In some embodiments, an antibody comprises an IgG1 Fc region comprising an 254I substitution, according to the Kabat numbering system. In some embodiments, an antibody comprises an IgG1 Fc region comprising an 254N substitution, according to the Kabat numbering system. In some embodiments, an antibody comprises an IgG1 Fc region comprising an 254P substitution, according to the Kabat numbering system. In some embodiments, an antibody comprises an IgG1 Fc region comprising an 254Q substitution, according to the Kabat numbering system. In some embodiments, an antibody comprises an IgG1 Fc region comprising an 254T substitution, according to the Kabat numbering system. In some embodiments, an antibody comprises an IgG1 Fe region comprising an 254V substitution, according to the Kabat numbering system. In some embodiments, an antibody comprises an IgG1 Fc region comprising an 255N substitution, according to the Kabat numbering system. In some embodiments, an antibody comprises an IgG1 Fc region comprising an 256H substitution, according to the Kabat numbering system. In some embodiments, an antibody comprises an IgG1 Fc region comprising an 256K substitution, according to the Kabat numbering system. In some embodiments, an antibody comprises an IgG1 Fc region comprising an 256R substitution, according to the Kabat numbering system. In some embodiments, an antibody comprises an IgG1 Fc region comprising an 256V substitution, according to the Kabat numbering system. In some embodiments, an antibody comprises an IgG1 Fc region comprising an 264S substitution, according to the Kabat numbering system. In some embodiments, an antibody comprises an IgG1 Fc region comprising an 265H substitution, according to the Kabat numbering system. In some embodiments, an antibody comprises an IgG1 Fc region comprising an 265K substitution, according to the Kabat numbering system. In some embodiments, an antibody comprises an IgG1 Fc region comprising an 265S substitution, according to the Kabat numbering system. In some embodiments, an antibody comprises an IgG1 Fc region comprising an 265Y substitution, according to the Kabat numbering system. In some embodiments, an antibody comprises an IgG1 Fc region comprising an 267G substitution, according to the Kabat numbering system. In some embodiments, an antibody comprises an IgG1 Fc region comprising an 267H substitution, according to the Kabat numbering system. In some embodiments, an antibody comprises an IgG1 Fc region comprising an 267I substitution, according to the Kabat numbering system. In some embodiments, an antibody comprises an IgG1 Fc region comprising an 267K substitution, according to the Kabat numbering system. In some embodiments, an antibody comprises an IgG1 Fc region comprising an 268K substitution, according to the Kabat numbering system. In some embodiments, an antibody comprises an IgG1 Fc region comprising an 269N substitution, according to the Kabat numbering system. In some embodiments, an antibody comprises an IgG1 Fc region comprising an 269Q substitution, according to the Kabat numbering system. In some embodiments, an antibody comprises an IgG1 Fc region comprising an 270A substitution, according to the Kabat numbering system. In some embodiments, an antibody comprises an IgG1 Fc region comprising an 270G substitution, according to the Kabat numbering system. In some embodiments, an antibody comprises an IgG1 Fc region comprising an 270M substitution, according to the Kabat numbering system. In some embodiments, an antibody comprises an IgG1 Fc region comprising an 270N substitution, according to the Kabat numbering system. In some embodiments, an antibody comprises an IgG1 Fc region comprising an 271T substitution, according to the Kabat numbering system. In some embodiments, an antibody comprises an IgG1 Fc region comprising an 272N substitution, according to the Kabat numbering system. In some embodiments, an antibody comprises an IgG1 Fc region comprising an 279F substitution, according to the Kabat numbering system. In some embodiments, an antibody comprises an IgG1 Fc region comprising an 279K substitution, according to the Kabat numbering system. In some embodiments, an antibody comprises an IgG1 Fc region comprising an 279L substitution, according to the Kabat numbering system. In some embodiments, an antibody comprises an IgG1 Fc region comprising an 292E substitution, according to the Kabat numbering system. In some embodiments, an antibody comprises an IgG1 Fc region comprising an 292F substitution, according to the Kabat numbering system. In some embodiments, an antibody comprises an IgG1 Fc region comprising an 292G substitution, according to the Kabat numbering system. In some embodiments, an antibody comprises an IgG1 Fc region comprising an 292I substitution, according to the Kabat numbering system. In some embodiments, an antibody comprises an IgG1 Fc region comprising an 293S substitution, according to the Kabat numbering system. In some embodiments, an antibody comprises an IgG1 Fc region comprising an 301 W substitution, according to the Kabat numbering system. In some embodiments, an antibody comprises an IgG1 Fc region comprising an 304E substitution, according to the Kabat numbering system. In some embodiments, an antibody comprises an IgG1 Fe region comprising an 311E substitution, according to the Kabat numbering system. In some embodiments, an antibody comprises an IgG1 Fe region comprising an 311G substitution, according to the Kabat numbering system. In some embodiments, an antibody comprises an IgG1 Fc region comprising an 311S substitution, according to the Kabat numbering system. In some embodiments, an antibody comprises an IgG1 Fc region comprising an 316F substitution, according to the Kabat numbering system. In some embodiments, an antibody comprises an IgG1 Fc region comprising an 327T substitution, according to the Kabat numbering system. In some embodiments, an antibody comprises an IgG1 Fc region comprising an 328V substitution, according to the Kabat numbering system. In some embodiments, an antibody comprises an IgG1 Fc region comprising an 329Y substitution, according to the Kabat numbering system. In some embodiments, an antibody comprises an IgG1 Fc region comprising an 330R substitution, according to the Kabat numbering system. In some embodiments, an antibody comprises an IgG1 Fc region comprising an 339E substitution, according to the Kabat numbering system. In some embodiments, an antibody comprises an IgG1 Fe region comprising an 339L substitution, according to the Kabat numbering system. In some embodiments, an antibody comprises an IgG1 Fc region comprising an 343I substitution, according to the Kabat numbering system. In some embodiments, an antibody comprises an IgG1 Fc region comprising an 343V substitution, according to the Kabat numbering system. In some embodiments, an antibody comprises an IgG1 Fc region comprising an 373A substitution, according to the Kabat numbering system. In some embodiments, an antibody comprises an IgG1 Fc region comprising an 373G substitution, according to the Kabat numbering system. In some embodiments, an antibody comprises an IgG1 Fc region comprising an 373S substitution, according to the Kabat numbering system. In some embodiments, an antibody comprises an IgG1 Fc region comprising an 376E substitution, according to the Kabat numbering system. In some embodiments, an antibody comprises an IgG1 Fc region comprising an 376W substitution, according to the Kabat numbering system. In some embodiments, an antibody comprises an IgG1 Fc region comprising an 376Y substitution, according to the Kabat numbering system. In some embodiments, an antibody comprises an IgG1 Fc region comprising an 380D substitution, according to the Kabat numbering system. In some embodiments, an antibody comprises an IgG1 Fc region comprising an 382D substitution, according to the Kabat numbering system. In some embodiments, an antibody comprises an IgG1 Fc region comprising an 382P substitution, according to the Kabat numbering system. In some embodiments, an antibody comprises an IgG1 Fc region comprising an 385P substitution, according to the Kabat numbering system. In some embodiments, an antibody comprises an IgG1 Fc region comprising an 424H substitution, according to the Kabat numbering system. In some embodiments, an antibody comprises an IgG1 Fc region comprising an 424M substitution, according to the Kabat numbering system. In some embodiments, an antibody comprises an IgG1 Fc region comprising an 424V substitution, according to the Kabat numbering system. In some embodiments, an antibody comprises an IgG1 Fc region comprising an 434I substitution, according to the Kabat numbering system. In some embodiments, an antibody comprises an IgG1 Fc region comprising an 438G substitution, according to the Kabat numbering system. In some embodiments, an antibody comprises an IgG1 Fc region comprising an 439E substitution, according to the Kabat numbering system. In some embodiments, an antibody comprises an IgG1 Fe region comprising an 439H substitution, according to the Kabat numbering system. In some embodiments, an antibody comprises an IgG1 Fc region comprising an 439Q substitution, according to the Kabat numbering system. In some embodiments, an antibody comprises an IgG1 Fc region comprising an 440A substitution, according to the Kabat numbering system. In some embodiments, an antibody comprises an IgG1 Fc region comprising an 440D substitution, according to the Kabat numbering system. In some embodiments, an antibody comprises an IgG1 Fc region comprising an 440E substitution, according to the Kabat numbering system. In some embodiments, an antibody comprises an IgG1 Fc region comprising an 440F substitution, according to the Kabat numbering system. In some embodiments, an antibody comprises an IgG1 Fc region comprising an 440M substitution, according to the Kabat numbering system. In some embodiments, an antibody comprises an IgG1 Fc region comprising an 440T Fc region substitution, according to the Kabat numbering system. In some embodiments, an antibody comprises an IgG1 Fc region comprising an 440V substitution, according to the Kabat numbering system.

In some embodiments, an antibody comprises a Fc region selected from the representative sequences disclosed in Table 3 or 20 In some embodiments, an antibody comprises an IgG1 Fc region comprising E233P, according to the Kabat numbering system. In some embodiments, an antibody comprises an IgG4 Fc region comprising S228P and L235E. In some embodiments, an antibody comprises an IgG1 Fc region comprising L235E, according to the Kabat numbering system. In some embodiments, an antibody comprises an IgG1 Fc region comprising L234A and L235A, according to the Kabat numbering system. In some embodiments, an antibody comprises an IgG1 Fc region comprising L234A, L235A, and G237A, according to the Kabat numbering system. In some embodiments, an antibody comprises an IgG1 Fc region comprising L234A, L235A, P329G, according to the Kabat numbering system. In some embodiments, an antibody comprises an IgG1 Fc region comprising L234F, L235E, and P331S, according to the Kabat numbering system. In some embodiments, an antibody comprises an IgG1 Fc region comprising L234A, L235E, and G237A, according to the Kabat numbering system. In some embodiments, an antibody comprises an IgG1 Fc region comprising L234A, L235E, G237A, and P331S, according to the Kabat numbering system. In some embodiments, an antibody comprises an IgG1 Fc region comprising L234A, L235A, G237A, P238S, H268A, A330S, and P331S (IgG1σ), according to the Kabat numbering system. In some embodiments, an antibody comprises an IgG1 Fc region comprising L234A, L235A, and P329A, according to the Kabat numbering system. In some embodiments, an antibody comprises an IgG1 Fc region comprising G236R and L328R, according to the Kabat numbering system. In some embodiments, an antibody comprises an IgG1 Fc region comprising G237A, according to the Kabat numbering system. In some embodiments, an antibody comprises an IgG1 Fc region comprising F241A, according to the Kabat numbering system. In some embodiments, an antibody comprises an IgG1 Fc region comprising V264A, according to the Kabat numbering system. In some embodiments, an antibody comprises an IgG1 Fc region comprising D265A, according to the Kabat numbering system. In some embodiments, an antibody comprises an IgG1 Fc region comprising D265A and N297A, according to the Kabat numbering system. In some embodiments, an antibody comprises an IgG1 Fc region comprising D265A and N297G, according to the Kabat numbering system. In some embodiments, an antibody comprises an IgG1 Fc region comprising D270A, according to the Kabat numbering system. In some embodiments, an antibody comprises an IgG1 Fc region comprising N297A, according to the Kabat numbering system. In some embodiments, an antibody comprises an IgG1 Fc region comprising N297G, according to the Kabat numbering system. In some embodiments, an antibody comprises an IgG1 Fc region comprising N297D, according to the Kabat numbering system. In some embodiments, an antibody comprises an IgG1 Fc region comprising N297Q, according to the Kabat numbering system. In some embodiments, an antibody comprises an IgG1 Fc region comprising P329A, according to the Kabat numbering system. In some embodiments, an antibody comprises an IgG1 Fc region comprising P329G, according to the Kabat numbering system. In some embodiments, an antibody comprises an IgG1 Fc region comprising P329R, according to the Kabat numbering system. In some embodiments, an antibody comprises an IgG1 Fc region comprising A330L, according to the Kabat numbering system. In some embodiments, an antibody comprises an IgG1 Fc region comprising P331A, according to the Kabat numbering system. In some embodiments, an antibody comprises an IgG1 Fc region comprising P331S, according to the Kabat numbering system. In some embodiments, an antibody comprises an IgG2 Fc region. In some embodiments, an antibody comprises an IgG4 Fc region. In some embodiments, an antibody comprises an IgG4 Fc region comprising S228P, according to the Kabat numbering system. In some embodiments, an antibody comprises an IgG4 Fc region comprising S228P, F234A, and L235A, according to the Kabat numbering system. In some embodiments, an antibody comprises an IgG2-IgG4 cross-subclass (IgG2/G4) Fc region. In some embodiments, an antibody comprises an IgG2-IgG3 cross-subclass Fc region. In some embodiments, an antibody comprises an IgG2 Fc region comprising H268Q, V309L, A330S, and P331S, according to the Kabat numbering system. In some embodiments, an antibody comprises an IgG2 Fc region comprising V234A, G237A, P238S, H268A, V309L, A330S, and P331S, according to the Kabat numbering system. In some embodiments, an antibody comprises a Fc region comprising high mannose glycosylation.

In some embodiments, an antibody comprises an IgG4 Fc region comprising a S228P substitution, according to the Kabat numbering system. In some embodiments, an antibody comprises an IgG4 Fc region comprising an A330S substitution, according to the Kabat numbering system. In some embodiments, an antibody comprises an IgG4 Fc region comprising a P331S substitution, according to the Kabat numbering system.

In some embodiments, an antibody comprises an IgG2 Fc region comprising an A330S substitution, according to the Kabat numbering system. In some embodiments, an antibody comprises an IgG2 Fc region comprising an P331S substitution, according to the Kabat numbering system. In some embodiments, an antibody comprises an IgG2 Fc region comprising an 234A substitution, according to the Kabat numbering system. In some embodiments, an antibody comprises an IgG2 Fc region comprising an 237A substitution, according to the Kabat numbering system.

In certain embodiments, an anti-TL1A antibody described herein comprises a Fc region comprising a sequence from Table 19. In certain embodiments, an anti-TL1A described herein comprises a Fc region as shown in Table 3.

TABLE 3
Exemplary Fc Mutations

| | Constant Region (SEQ ID NO) | | |
|---|---|---|---|
| Mutations | K_DL | R_EM | K_EM |
| Wild-type IgG1 | 320 | 321 | 322 |
| L235E | 323 | 324 | 325 |
| L234A, L235A | 326 | 327 | 328 |
| L234A, L235A, G237A | 329 | 330 | 331 |
| L234A, L235A, P329G | 332 | 333 | 334 |
| L234F, L235E, P331S | 335 | 336 | 337 |
| L234A, L235E, G237A | 338 | 339 | 340 |
| L234A, L235E, G237A, P331S | 341 | 342 | 343 |
| L234A, L235A, P329A | 344 | 345 | 346 |
| D265A | 347 | 348 | 349 |
| N297G | 350 | 351 | 352 |
| D265A, N297A | 353 | 354 | 355 |
| D265A, N297G | 356 | 357 | 358 |
| L235A, G237A | 359 | 360 | 361 |
| Wild-type IgG4 | 362 | | |

In certain embodiments, an anti-TL1A antibody described herein comprises a Fc region comprising SEQ ID NO: 320 or a sequence at least about 90%, 91%, 92%, 93%, 94%, 95%, 96%, 97%, 98%, or 99% identical to SEQ ID NO: 320. In certain embodiments, an anti-TL1A antibody described herein comprises a Fc region comprising SEQ ID NO: 321 or a sequence at least about 90%, 91%, 92%, 93%, 94%, 95%, 96%, 97%, 98%, or 99% identical to SEQ ID NO: 321. In certain embodiments, an anti-TL1A antibody described herein comprises a Fc region comprising SEQ ID NO: 322 or a sequence at least about 90%, 91%, 92%, 93%, 94%, 95%, 96%, 97%, 98%, or 99% identical to SEQ ID NO: 322. In certain embodiments, an anti-TL1A antibody described herein comprises a Fc region comprising SEQ ID NO: 323 or a sequence at least about 90%, 91%, 92%, 93%, 94%, 95%, 96%, 97%, 98%, or 99% identical to SEQ ID NO: 323. In certain embodiments, an anti-TL1A antibody described herein comprises a Fc region comprising SEQ ID NO: 324 or a sequence at least about 90%, 91%, 92%, 93%, 94%, 95%, 96%, 97%, 98%, or 99% identical to SEQ ID NO: 324. In certain embodiments, an anti-TL1A antibody described herein comprises a Fc region comprising SEQ ID NO: 325 or a sequence at least about 90%, 91%, 92%, 93%, 94%, 95%, 96%, 97%, 98%, or 99% identical to SEQ ID NO: 325. In certain embodiments, an anti-TL1A antibody described herein comprises a Fc region comprising SEQ ID NO: 326 or a sequence at least about 90%, 91%, 92%, 93%, 94%, 95%, 96%, 97%, 98%, or 99% identical to SEQ ID NO: 326. In certain embodiments, an anti-TL1A antibody described herein comprises a Fc region comprising SEQ ID NO: 327 or a sequence at least about 90%, 91%, 92%, 93%, 94%, 95%, 96%, 97%, 98%, or 99% identical to SEQ ID NO: 327. In certain embodiments, an anti-TL1A antibody described herein comprises a Fc region comprising SEQ ID NO: 328 or a sequence at least about 90%, 91%, 92%, 93%, 94%, 95%, 96%, 97%, 98%, or 99% identical to SEQ ID NO: 328. In certain embodiments, an anti-TL1A antibody described herein comprises a Fc region comprising SEQ ID NO: 329 or a sequence at least about 90%, 91%, 92%, 93%, 94%, 95%, 96%, 97%, 98%, or 99% identical to SEQ ID NO: 329. In certain embodiments, an anti-TL1A antibody described herein comprises a Fc region comprising SEQ ID NO: 330 or a sequence at least about 90%, 91%, 92%, 93%, 94%, 95%, 96%, 97%, 98%, or 99% identical to SEQ ID NO: 330. In certain embodiments, an anti-TL1A antibody described herein comprises a Fc region comprising SEQ ID NO: 331 or a sequence at least about 90%, 91%, 92%, 93%, 94%, 95%, 96%, 97%, 98%, or 99% identical to SEQ ID NO: 331. In certain embodiments, an anti-TL1A antibody described herein comprises a Fc region comprising SEQ ID NO: 332 or a sequence at least about 90%, 91%, 92%, 93%, 94%, 95%, 96%, 97%, 98%, or 99% identical to SEQ ID NO: 332. In certain embodiments, an anti-TL1A antibody described herein comprises a Fc region comprising SEQ ID NO: 333 or a sequence at least about 90%, 91%, 92%, 93%, 94%, 95%, 96%, 97%, 98%, or 99% identical to SEQ ID NO: 333. In certain embodiments, an anti-TL1A antibody described herein comprises a Fc region comprising SEQ ID NO: 334 or a sequence at least about 90%, 91%, 92%, 93%, 94%, 95%, 96%, 97%, 98%, or 99% identical to SEQ ID NO: 334. In certain embodiments, an anti-TL1A antibody described herein comprises a Fc region comprising SEQ ID NO: 335 or a sequence at least about 90%, 91%, 92%, 93%, 94%, 95%, 96%, 97%, 98%, or 99% identical to SEQ ID NO: 335. In certain embodiments, an anti-TL1A antibody described herein comprises a Fc region comprising SEQ ID NO: 336 or a sequence at least about 90%, 91%, 92%, 93%, 94%, 95%, 96%, 97%, 98%, or 99% identical to SEQ ID NO: 336. In certain embodiments, an anti-TL1A antibody described herein comprises a Fc region comprising SEQ ID NO: 337 or a sequence at least about 90%, 91%, 92%, 93%, 94%, 95%, 96%, 97%, 98%, or 99% identical to SEQ ID NO: 337. In certain embodiments, an anti-TL1A antibody described herein comprises a Fc region comprising SEQ ID NO: 338 or a sequence at least about 90%, 91%, 92%, 93%, 94%, 95%, 96%, 97%, 98%, or 99% identical to SEQ ID NO: 338. In certain embodiments, an anti-TL1A antibody described herein comprises a Fc region comprising SEQ ID NO: 339 or a sequence at least about 90%, 91%, 92%, 93%, 94%, 95%, 96%, 97%, 98%, or 99% identical to SEQ ID NO: 339. In certain embodiments, an anti-TL1A antibody described herein comprises a Fc region comprising SEQ ID NO: 340 or a sequence at least about 90%, 91%, 92%, 93%, 94%, 95%, 96%, 97%, 98%, or 99% identical to SEQ ID NO: 340. In certain embodiments, an anti-TL1A antibody described herein comprises a Fc region comprising SEQ ID NO: 341 or a sequence at least about 90%, 91%, 92%, 93%, 94%, 95%, 96%, 97%, 98%, or 99% identical to SEQ ID NO: 341. In certain embodiments, an anti-TL1A antibody described herein comprises a Fc region comprising SEQ ID NO: 342 or a sequence at least about 90%, 91%, 92%, 93%, 94%, 95%, 96%, 97%, 98%, or 99% identical to SEQ ID NO: 342. In certain embodiments, an anti-TL1A antibody described herein comprises a Fc region comprising SEQ ID NO: 343 or a sequence at least about 90%, 91%, 92%, 93%, 94%, 95%, 96%, 97%, 98%, or 99% identical to SEQ ID NO: 343. In certain embodiments, an anti-TL1A antibody described herein comprises a Fc region comprising SEQ ID NO: 344 or a sequence at least about 90%, 91%, 92%, 93%, 94%, 95%, 96%, 97%, 98%, or 99% identical to SEQ ID NO: 344. In certain embodiments, an anti-TL1A antibody described herein comprises a Fc region comprising SEQ ID NO: 345 or a sequence at least about 90%, 91%, 92%, 93%, 94%, 95%, 96%, 97%, 98%, or 99% identical to SEQ ID NO: 345. In certain embodiments, an anti-TL1A antibody described herein comprises a Fc region comprising SEQ ID NO: 346 or a sequence at least about 90%, 91%, 92%, 93%, 94%, 95%, 96%, 97%, 98%, or 99% identical to SEQ ID NO: 346. In certain embodiments, an anti-TL1A antibody described herein comprises a Fc region comprising SEQ ID NO: 347 or a sequence at least about 90%, 91%, 92%, 93%, 94%, 95%, 96%, 97%, 98%, or 99% identical to SEQ ID NO: 347. In certain embodiments, an anti-TL1A antibody described herein comprises a Fc region comprising SEQ ID NO: 348 or a sequence at least about 90%, 91%, 92%, 93%, 94%, 95%, 96%, 97%, 98%, or 99% identical to SEQ ID NO: 348. In certain embodiments, an anti-TL1A antibody described herein comprises a Fc region comprising SEQ ID NO: 349 or a sequence at least about 90%, 91%, 92%, 93%, 94%, 95%, 96%, 97%, 98%, or 99% identical to SEQ ID NO: 349. In certain embodiments, an anti-TL1A antibody described herein comprises a Fc region comprising SEQ ID NO: 350 or a sequence at least about 90%, 91%, 92%, 93%, 94%, 95%, 96%, 97%, 98%, or 99% identical to SEQ ID NO: 350. In certain embodiments, an anti-TL1A antibody described herein comprises a Fc region comprising SEQ ID NO: 351 or a sequence at least about 90%, 91%, 92%, 93%, 94%, 95%, 96%, 97%, 98%, or 99% identical to SEQ ID NO: 351. In certain embodiments, an anti-TL1A antibody described herein comprises a Fc region comprising SEQ ID NO: 352 or a sequence at least about 90%, 91%, 92%, 93%, 94%, 95%, 96%, 97%, 98%, or 99% identical to SEQ ID NO: 352. In certain embodiments, an anti-TL1A antibody described herein comprises a Fc region comprising SEQ ID NO: 353 or a sequence at least about 90%, 91%, 92%, 93%, 94%, 95%, 96%, 97%, 98%, or 99% identical to SEQ ID NO: 353. In certain embodiments, an anti-TL1A antibody described herein comprises a Fc region comprising SEQ ID NO: 354 or a sequence at least about 90%, 91%, 92%, 93%, 94%, 95%, 96%, 97%, 98%, or 99% identical to SEQ ID NO: 354. In certain embodiments, an anti-TL1A antibody described herein comprises a Fe region comprising SEQ ID NO: 355 or a sequence at least about 90%, 91%, 92%, 93%, 94%, 95%, 96%, 97%, 98%, or 99% identical to SEQ ID NO: 355. In certain embodiments, an anti-TL1A antibody described herein comprises a Fc region comprising SEQ ID NO: 356 or a sequence at least about 90%, 91%, 92%, 93%, 94%, 95%, 96%, 97%, 98%, or 99% identical to SEQ ID NO: 356. In certain embodiments, an anti-TL1A antibody described herein comprises a Fc region comprising SEQ ID NO: 357 or a sequence at least about 90%, 91%, 92%, 93%, 94%, 95%, 96%, 97%, 98%, or 99% identical to SEQ ID NO: 357. In certain embodiments, an anti-TL1A antibody described herein comprises a Fc region comprising SEQ ID NO: 358 or a sequence at least about 90%, 91%, 92%, 93%, 94%, 95%, 96%, 97%, 98%, or 99% identical to SEQ ID NO: 358. In certain embodiments, an anti-TL1A antibody described herein comprises a Fc region comprising SEQ ID NO: 359 or a sequence at least about 90%, 91%, 92%, 93%, 94%, 95%, 96%, 97%, 98%, or 99% identical to SEQ ID NO: 359. In certain embodiments, an anti-TL1A antibody described herein comprises a Fc region comprising SEQ ID NO: 360 or a sequence at least about 90%, 91%, 92%, 93%, 94%, 95%, 96%, 97%, 98%, or 99% identical to SEQ ID NO: 360. In certain embodiments, an anti-TL1A antibody described herein comprises a Fc region comprising SEQ ID NO: 361 or a sequence at least about 90%, 91%, 92%, 93%, 94%, 95%, 96%, 97%, 98%, or 99% identical to SEQ ID NO: 361. In certain embodiments, an anti-TL1A antibody described herein comprises a Fc region comprising SEQ ID NO: 362 or a sequence at least about 90%, 91%, 92%, 93%, 94%, 95%, 96%, 97%, 98%, or 99% identical to SEQ ID NO: 362.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. Measurement of effector function may be performed as described in Example 3.

In some embodiments, antibodies are tested for binding to Fcγ receptors and complement C1q by ELISA. In some embodiments, antibodies are tested for the ability to activate primary human immune cells in vitro, for example, by assessing their ability to induce expression of activation markers.

In some embodiments, assessment of ADCC activity of an anti-TL1A antibody comprises adding the antibody to target cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361, Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, antibodies comprising a Fc region herein exhibit decreased ADCC activities as compared to an unmodified antibody (e.g., an antibody with human IgG1). In some embodiments, the antibodies herein exhibit ADCC activities that are at least 2-fold, or at least 3-fold, or at least 5-fold or at least 10-fold or at least 50-fold or at least 100-fold less than that of an unmodified antibody. In some embodiments, antibodies herein exhibit ADCC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein have no detectable ADCC activity. In certain embodiments, the reduction and/or abatement of ADCC activity may be attributed to the reduced affinity antibodies of the invention exhibit for Fc ligands and/or receptors.

In some embodiments, an assessment of complement activation, a CDC assay, may be performed as described in Gazzano-Santoro et al., 1996, J. Immunol. Methods, 202:163.

In some embodiments, antibodies comprising Fc regions described herein exhibit decreased affinities to C1q relative to an unmodified antibody (e.g., human IgG1). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody. In some embodiments, antibodies herein exhibit affinities for C1q that are between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, antibodies herein exhibit affinities for C1q that are greater than 1 μM, greater than 5 μM, greater than 10 μM, greater than 25 μM, greater than 50 μM, or greater than 100 μM.

In some embodiments, antibodies comprising Fc regions described herein exhibit decreased CDC activities as compared to an unmodified antibody (e.g., human IgG1). In some embodiments, antibodies herein exhibit CDC activities that are at least 2-fold, or at least 3-fold, or at least 5-fold or at least 10-fold or at least 50-fold or at least 100-fold less than that of an unmodified antibody. In some embodiments, antibodies herein exhibit CDC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein exhibit no detectable CDC activities. In some embodiments, the reduction and/or abatement of CDC activity may be attributed to the reduced affinity antibodies of the invention exhibit for Fc ligands and/or receptors.

Accordingly, further provided and described herein are anti-TL1A antibodies comprising a variant (e.g. harboring mutations) Fc region that reduce the cytotoxic response (e.g. ADCC or CDC) elicited by an anti-TL1A antibody. In some embodiments, an anti-TL1A antibody described herein comprises a Fc region comprising SEQ ID NO: 401 or a sequence at least about 90%, 91%, 92%, 93%, 94%, 95%, 96%, 97%, 98%, or 99% identical to SEQ ID NO: 401. In some embodiments, an anti-TL1A antibody described herein comprises a Fc region comprising SEQ ID NO: 402 or a sequence at least about 90%, 91%, 92%, 93%, 94%, 95%, 96%, 97%, 98%, or 99% identical to SEQ ID NO: 402. In some embodiments, an anti-TL1A antibody described herein comprises a Fc region comprising SEQ ID NO: 403 or a sequence at least about 90%, 91%, 92%, 93%, 94%, 95%, 96%, 97%, 98%, or 99% identical to SEQ ID NO: 403. In some embodiments, an anti-TL1A antibody described herein comprises a Fc region comprising SEQ ID NO: 404 or a sequence at least about 90%, 91%, 92%, 93%, 94%, 95%, 96%, 97%, 98%, or 99% identical to SEQ ID NO: 404. In some embodiments, an anti-TL1A antibody described herein comprises a Fc region comprising SEQ ID NO: 405 or a sequence at least about 90%, 91%, 92%, 93%, 94%, 95%, 96%, 97%, 98%, or 99% identical to SEQ ID NO: 405. In some embodiments, an anti-TL1A antibody described herein comprises a Fc region comprising SEQ ID NO: 406 or a sequence at least about 90%, 91%, 92%, 93%, 94%, 95%, 96%, 97%, 98%, or 99% identical to SEQ ID NO: 406. In some embodiments, an anti-TL1A antibody described herein comprises a Fc region comprising SEQ ID NO: 407 or a sequence at least about 90%, 91%, 92%, 93%, 94%, 95%, 96%, 97%, 98%, or 99% identical to SEQ ID NO: 407. In some embodiments, an anti-TL1A antibody described herein comprises a Fc region comprising SEQ ID NO: 408 or a sequence at least about 90%, 91%, 92%, 93%, 94%, 95%, 96%, 97%, 98%, or 99% identical to SEQ ID NO: 408. In some embodiments, an anti-TL1A antibody described herein comprises a Fc region comprising SEQ ID NO: 409 or a sequence at least about 90%, 91%, 92%, 93%, 94%, 95%, 96%, 97%, 98%, or 99% identical to SEQ ID NO: 409. In some embodiments, an anti-TL1A antibody described herein comprises a Fc region comprising SEQ ID NO: 410 or a sequence at least about 90%, 91%, 92%, 93%, 94%, 95%, 96%, 97%, 98%, or 99% identical to SEQ ID NO: 410. In some embodiments, an anti-TL1A antibody described herein comprises a Fc region comprising SEQ ID NO: 411 or a sequence at least about 90%, 91%, 92%, 93%, 94%, 95%, 96%, 97%, 98%, or 99% identical to SEQ ID NO: 411. In some embodiments, an anti-TL1A antibody described herein comprises a Fc region comprising SEQ ID NO: 412 or a sequence at least about 90%, 91%, 92%, 93%, 94%, 95%, 96%, 97%, 98%, or 99% identical to SEQ ID NO: 412. In some embodiments, an anti-TL1A antibody described herein comprises a Fc region comprising SEQ ID NO: 413 or a sequence at least about 90%, 91%, 92%, 93%, 94%, 95%, 96%, 97%, 98%, or 99% identical to SEQ ID NO: 413.

By way of further example, in certain embodiments, an anti-TL1A antibody described herein comprises a heavy chain comprising SEQ ID NO: 501 or a sequence at least about 90%, 91%, 92%, 93%, 94%, 95%, 96%, 97%, 98%, or 99% identical to SEQ ID NO: 501. In certain embodiments, an anti-TL1A antibody described herein comprises a heavy chain comprising SEQ ID NO: 502 or a sequence at least about 90%, 91%, 92%, 93%, 94%, 95%, 96%, 97%, 98%, or 99% identical to SEQ ID NO: 502. In certain embodiments, an anti-TL1A antibody described herein comprises a heavy chain comprising SEQ ID NO: 503 or a sequence at least about 90%, 91%, 92%, 93%, 94%, 95%, 96%, 97%, 98%, or 99% identical to SEQ ID NO: 503. In certain embodiments, an anti-TL1A antibody described herein comprises a heavy chain comprising SEQ ID NO: 504 or a sequence at least about 90%, 91%, 92%, 93%, 94%, 95%, 96%, 97%, 98%, or 99% identical to SEQ ID NO: 504. In certain embodiments, an anti-TL1A antibody described herein comprises a heavy chain comprising SEQ ID NO: 505 or a sequence at least about 90%, 91%, 92%, 93%, 94%, 95%, 96%, 97%, 98%, or 99% identical to SEQ ID NO: 505. In certain embodiments, an anti-TL1A antibody described herein comprises a heavy chain comprising SEQ ID NO: 506 or a sequence at least about 90%, 91%, 92%, 93%, 94%, 95%, 96%, 97%, 98%, or 99% identical to SEQ ID NO: 506. In certain embodiments, an anti-TL1A antibody described herein comprises a heavy chain comprising SEQ ID NO: 507 or a sequence at least about 90%, 91%, 92%, 93%, 94%, 95%, 96%, 97%, 98%, or 99% identical to SEQ ID NO: 507. In certain embodiments, an anti-TL1A antibody described herein comprises a heavy chain comprising SEQ ID NO: 508 or a sequence at least about 90%, 91%, 92%, 93%, 94%, 95%, 96%, 97%, 98%, or 99% identical to SEQ ID NO: 508. In certain embodiments, an anti-TL1A antibody described herein comprises a heavy chain comprising SEQ ID NO: 509 or a sequence at least about 90%, 91%, 92%, 93%, 94%, 95%, 96%, 97%, 98%, or 99% identical to SEQ ID NO: 509. In certain embodiments, an anti-TL1A antibody described herein comprises a heavy chain comprising SEQ ID NO: 510 or a sequence at least about 90%, 91%, 92%, 93%, 94%, 95%, 96%, 97%, 98%, or 99% identical to SEQ ID NO: 510. In certain embodiments, an anti-TL1A antibody described herein comprises a heavy chain comprising SEQ ID NO: 511 or a sequence at least about 90%, 91%, 92%, 93%, 94%, 95%, 96%, 97%, 98%, or 99% identical to SEQ ID NO: 511. In certain embodiments, an anti-TL1A antibody described herein comprises a heavy chain comprising SEQ ID NO: 512 or a sequence at least about 90%, 91%, 92%, 93%, 94%, 95%, 96%, 97%, 98%, or 99% identical to SEQ ID NO: 512. In certain embodiments, an anti-TL1A antibody described herein comprises a heavy chain comprising SEQ ID NO: 513 or a sequence at least about 90%, 91%, 92%, 93%, 94%, 95%, 96%, 97%, 98%, or 99% identical to SEQ ID NO: 513. In certain embodiments, the heavy chain is paired with a light chain comprising SEQ ID NO: 514 or a sequence at least about 90%, 91%, 92%, 93%, 94%, 95%, 96%, 97%, 98%, or 99% identical to SEQ ID NO: 514. In certain embodiments, the heavy chain is paired with the light chain variable region of SEQ ID NO: 514 or a sequence at least about 90%, 91%, 92%, 93%, 94%, 95%, 96%, 97%, 98%, or 99% identical to the light chain variable region of SEQ ID NO: 514. In certain embodiments, the heavy chain is paired with the light chain variable region of SEQ ID NO: 514 or a sequence at least about 90%, 91%, 92%, 93%, 94%, 95%, 96%, 97%, 98%, or 99% identical to the light chain variable region of SEQ ID NO: 515.

In some embodiments, anti-TL1A described herein comprise a light chain constant region comprising SEQ ID NO: 319 or a sequence at least about 90%, 91%, 92%, 93%, 94%, 95%, 96%, 97%, 98%, or 99% identical to SEQ ID NO: 319.

### Further Non-Limiting Exemplary Anti-TL1A Antibodies

In one aspect, provided herein is a first embodiment of an anti-TL1A antibody comprising a heavy chain comprising a HCDR1, a HCDR2, and a HCDR3, and a light chain comprising a LCDR1, a LCDR2, and a LCDR3. Non-limiting additional embodiments include: (Embodiment 2) The anti-TL1A antibody of embodiment 1, comprising a HCDR1 comprising SEQ ID NO: 1. (Embodiment 3) The anti-TL1A antibody of embodiment 1 or embodiment 2, comprising a HCDR2 comprising SEQ ID NO: 2. (Embodiment 4) The anti-TL1A antibody of embodiment 1 or embodiment 2, comprising a HCDR2 comprising SEQ ID NO: 3. (Embodiment 5) The anti-TL1A antibody of embodiment 1 or embodiment 2, comprising a HCDR2 comprising SEQ ID NO: 4. (Embodiment 6) The anti-TL1A antibody of embodiment 1 or embodiment 2, comprising a HCDR2 comprising SEQ ID NO: 5. (Embodiment 7) The anti-TL1A antibody of any one of embodiments 1-6, comprising a HCDR3 comprising SEQ ID NO: 6. (Embodiment 8) The anti-TL1A antibody of any one of embodiments 1-6, comprising a HCDR3 comprising SEQ ID NO: 7. (Embodiment 9) The anti-TL1A antibody of any one of embodiments 1-6, comprising a HCDR3 comprising SEQ ID NO: 8. (Embodiment 10) The anti-TL1A antibody of any one of embodiments 1-6, comprising a HCDR3 comprising SEQ ID NO: 9. (Embodiment 11) The anti-TL1A antibody of any one of embodiments 1-10, comprising a LCDR1 comprising SEQ ID NO: 10. (Embodiment 12) The anti-TL1A antibody of any one of embodiments 1-11, comprising a LCDR2 comprising SEQ ID NO: 11. (Embodiment 13) The anti-TL1A antibody of any one of embodiments 1-12, comprising a LCDR3 comprising SEQ ID NO: 12. (Embodiment 14) The anti-TL1A antibody of any one of embodiments 1-12, comprising a LCDR3 comprising SEQ ID NO: 13. (Embodiment 15) The anti-TL1A antibody of any one of embodiments 1-12, comprising a LCDR3 comprising SEQ ID NO: 14 or 15.

(Embodiment 16) The anti-TL1A antibody of any one of embodiments 1-15, comprising a heavy chain framework comprising IGHV1-46*02. (Embodiment 17) The anti-TL1A antibody of any one of embodiments 1-15, comprising a heavy chain framework comprising a variant of IGHV1-46*02 comprising between about 1 and about 20 amino acid substitutions from SEQ ID NO: 316. (Embodiment 18) The anti-TL1A antibody of any one of embodiments 1-15, comprising a heavy chain framework comprising a variant of IGHV1-46*02 comprising between about 1 and about 9 amino acid substitutions from SEQ ID NO: 316. (Embodiment 19) The anti-TL1A antibody of any one of embodiments 1-15, comprising a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. (Embodiment 20) The anti-TL1A antibody of any one of embodiments 17-19, wherein the heavy chain framework substitution comprises Q1E, as determined by Aho or Kabat numbering. (Embodiment 21) The anti-TL1A antibody of any one of embodiments 17-20, wherein the heavy chain framework substitution comprises R45K, as determined by Aho or Kabat numbering. (Embodiment 22) The anti-TL1A antibody of any one of embodiments 17-21, wherein the heavy chain framework substitution comprises A47R, as determined by Aho or Kabat numbering. (Embodiment 23) The anti-TL1A antibody of any one of embodiments 17-22, wherein the heavy chain framework substitution comprises M55I, as determined by Aho or Kabat numbering. (Embodiment 24) The anti-TL1A antibody of any one of embodiments 17-23, wherein the heavy chain framework substitution comprises V78A, as determined by Aho or Kabat numbering. (Embodiment 25) The anti-TL1A antibody of any one of embodiments 17-24, wherein the heavy chain framework substitution comprises M80I, as determined by Aho or Kabat numbering. (Embodiment 26) The anti-TL1A antibody of any one of embodiments 17-25, wherein the heavy chain framework substitution comprises R82T, as determined by Aho or Kabat numbering. (Embodiment 27) The anti-TL1A antibody of any one of embodiments 17-26, wherein the heavy chain framework substitution comprises V89A, as determined by Aho or Kabat numbering. (Embodiment 28) The anti-TL1A antibody of any one of embodiments 17-27, wherein the heavy chain framework substitution comprises M91L, as determined by Aho or Kabat numbering.

(Embodiment 29) The anti-TL1A antibody of any one of embodiments 1-15, comprising a heavy chain framework comprising SEQ ID NO: 301. (Embodiment 30) The anti-TL1A antibody of embodiment 29, wherein X1 is Q. (Embodiment 31) The anti-TL1A of embodiment 29, wherein X1=E. (Embodiment 32) The anti-TL1A of any one of embodiments 29-31, wherein X2=R. (Embodiment 33) The anti-TL1A of any one of embodiments 29-31, wherein X2=K. (Embodiment 34) The anti-TL1A of any one of embodiments 29-33, wherein X3=A. (Embodiment 35) The anti-TL1A of any one of embodiments 29-33, wherein X3=R. (Embodiment 36) The anti-TL1A of any one of embodiments 29-35, wherein X4=M. (Embodiment 37) The anti-TL1A of any one of embodiments 29-35, wherein X4=I. (Embodiment 38) The anti-TL1A of any one of embodiments 29-37, wherein X5=V. (Embodiment 39) The anti-TL1A of any one of embodiments 29-37, wherein X5=A. (Embodiment 40) The anti-TL1A of any one of embodiments 29-39, wherein X6=M. (Embodiment 41) The anti-TL1A of any one of embodiments 29-39, wherein X6=I. (Embodiment 42) The anti-TL1A of any one of embodiments 29-41, wherein X7=R. (Embodiment 43) The anti-TL1A of any one of embodiments 29-41, wherein X7=T. (Embodiment 44) The anti-TL1A of any one of embodiments 29-43, wherein X8=V. (Embodiment 45) The anti-TL1A of any one of embodiments 29-43, wherein X8=A. (Embodiment 46) The anti-TL1A of any one of embodiments 29-45, wherein X9=M. (Embodiment 47) The anti-TL1A of any one of embodiments 29-45, wherein X9=L.

(Embodiment 48) The anti-TL1A antibody of any one of embodiments 1-15, comprising a heavy chain framework comprising SEQ ID NO: 302. (Embodiment 49) The anti-TL1A antibody of embodiment 48, wherein X1 is Q. (Embodiment 50) The anti-TL1A of embodiment 48, wherein X1=E. (Embodiment 51) The anti-TL1A of any one of embodiments 48-50, wherein X2=R. (Embodiment 52) The anti-TL1A of any one of embodiments 48-50, wherein X2=K. (Embodiment 53) The anti-TL1A of any one of embodiments 48-52, wherein X3=A. (Embodiment 54) The anti-TL1A of any one of embodiments 48-52, wherein X3=R. (Embodiment 55) The anti-TL1A of any one of embodiments 48-54, wherein X4=M. (Embodiment 56) The anti-TL1A of any one of embodiments 48-54, wherein X4=I. (Embodiment 57) The anti-TL1A of any one of embodiments 48-56, wherein X5=V. (Embodiment 58) The anti-TL1A of any one of embodiments 48-56, wherein X5=A. (Embodiment 59) The anti-TL1A of any one of embodiments 48-58, wherein X6=M. (Embodiment 60) The anti-TL1A of any one of embodiments 48-58, wherein X6=I. (Embodiment 61) The anti-TL1A of any one of embodiments 48-60, wherein X7=R. (Embodiment 62) The anti-TL1A of any one of embodiments 48-60, wherein X7=T. (Embodiment 63) The anti-TL1A of any one of embodiments 48-62, wherein X8=V. (Embodiment 64) The anti-TL1A of any one of embodiments 48-62, wherein X8=A. (Embodiment 65) The anti-TL1A of any one of embodiments 48-64, wherein X9=M. (Embodiment 66) The anti-TL1A of any one of embodiments 48-64, wherein X9=L.

(Embodiment 67) The anti-TL1A antibody of any one of embodiments 1-66, comprising a light chain framework comprising IGKV3-20*01. (Embodiment 68) The anti-TL1A antibody of any one of embodiments 1-66, comprising a light chain framework comprising a variant of IGKV3-20*01 comprising between about 1 and about 20 amino acid substitutions from SEQ ID NO: 317. (Embodiment 69) The anti-TL1A antibody of any one of embodiments 1-66, comprising a light chain framework comprising a variant of IGKV3-20*01 comprising about 1 amino acid substitution from SEQ ID NO: 317. (Embodiment 70) The anti-TL1A antibody of any one of embodiments 1-66, comprising a light chain framework comprising a variant of IGKV3-20*01 comprising about 2 amino acid substitutions from SEQ ID NO: 317. (Embodiment 71) The anti-TL1A antibody of any one of embodiments 1-66, comprising a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. (Embodiment 72) The anti-TL1A antibody of any one of embodiments 69-71, wherein the light chain framework substitution comprises Q1E, as determined by Aho or Kabat numbering. (Embodiment 73) The anti-TL1A antibody of any one of embodiments 69-72, wherein the light chain framework substitution comprises R45K, as determined by Aho or Kabat numbering.

(Embodiment 74) The anti-TL1A antibody of any one of embodiments 1-66, comprising a light chain comprising a light chain framework comprising SEQ ID NO: 303. (Embodiment 75) The anti-TL1A antibody of embodiment 74, wherein X10 is L. (Embodiment 76) The anti-TL1A antibody of embodiment 74, wherein X10 is P. (Embodiment 77) The anti-TL1A antibody of any one of embodiments 74-76, wherein X11 is L. (Embodiment 78) The anti-TL1A antibody of any one of embodiments 74-76, wherein X11 is W.

(Embodiment 79) The anti-TL1A of any one of embodiments 1-78, comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. (Embodiment 80) The anti-TL1A of any one of embodiments 1-78, comprising a (i) human IgG4 Fc region or (ii) a human IgG4 Fc region comprising (a) S228P and L235E, or (b) S228P, F234A, and L235A, per Kabat numbering. (Embodiment 81) The anti-TL1A of any one of embodiments 1-78, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ (Embodiment 82) The anti-TL1A of any one of embodiments 1-81, comprising a heavy chain Fc region comprising any one of SEQ ID NOS: 320-362. (Embodiment 83) The anti-TL1A antibody of any one of embodiments 1-82, comprising a light chain constant region comprising SEQ ID NO: 319.

(Embodiment 84) The anti-TL1A antibody of any one of embodiments 1-83, comprising at least about 80% monomeric fraction as determined by the size exclusion chromatography method described herein. (Embodiment 85) The anti-TL1A antibody of any one of embodiments 1-84, comprising at least about 81%, at least about 82%, at least about 83%, or at least about 84% monomeric fraction as determined by the size exclusion chromatography method described herein. (Embodiment 86) The anti-TL1A antibody of any one of embodiments 1-85, comprising at least about 85% monomeric fraction as determined by the size exclusion chromatography method described herein. (Embodiment 87) The anti-TL1A antibody of any one of embodiments 1-86, comprising at least about 86%, at least about 87%, at least about 88%, or at least about 89% monomeric fraction as determined by the size exclusion chromatography method described herein. (Embodiment 88) The anti-TL1A antibody of any one of embodiments 1-87, comprising at least about 90% monomeric fraction as determined by the size exclusion chromatography method described herein. (Embodiment 89) The anti-TL1A antibody of any one of embodiments 1-88, comprising at least about 91%, at least about 92%, at least about 93%, or at least about 94% monomeric fraction as determined by the size exclusion chromatography method described herein. (Embodiment 90) The anti-TL1A antibody of any one of embodiments 1-89, comprising at least about 95% monomeric fraction as determined by the size exclusion chromatography method described herein. (Embodiment 91) The anti-TL1A antibody of any one of embodiments 1-90, comprising at least about 96%, at least about 97%, at least about 98%, or at least about 99% monomeric fraction as determined by the size exclusion chromatography method described herein.

(Embodiment 92) The anti-TL1A antibody of any one of embodiments 1-91, comprising at least about 2 μg/mL expression as determined by the method disclosed herein. (Embodiment 93) The anti-TL1A antibody of any one of embodiments 1-92, comprising between about 2 μg/mL and about 60 μg/mL expression as determined by the method disclosed herein. (Embodiment 94) The anti-TL1A antibody of any one of embodiments 1-93, comprising between about 5 μg/mL and about 60 μg/mL expression as determined by the method disclosed herein. (Embodiment 95) The anti-TL1A antibody of any one of embodiments 1-94, comprising between about 10 μg/mL and about 60 μg/mL expression as determined by the method disclosed herein. (Embodiment 96) The anti-TL1A antibody of any one of embodiments 1-95, comprising at least about 5 μg/mL expression as determined by the method disclosed herein. (Embodiment 97) The anti-TL1A antibody of any one of embodiments 1-96, comprising at least about 10 μg/mL expression as determined by the method disclosed herein. (Embodiment 98) The anti-TL1A antibody of any one of embodiments 1-97, comprising at least about 15 μg/mL expression as determined by the method disclosed herein. (Embodiment 99) The anti-TL1A antibody of any one of embodiments 1-98, comprising at least about 20 μg/mL expression as determined by the method disclosed herein. (Embodiment 100) The anti-TL1A antibody of any one of embodiments 1-91, comprising between about 2 μg/mL and about 50 μg/mL, between about 2 μg/mL and about 40 μg/mL, between about 2 μg/mL and about 30 μg/mL expression, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL as determined by the method disclosed herein. (Embodiment 101) The anti-TL1A antibody of any one of embodiments 1-91, comprising about 2, 3, 4, 5, 6, 7, 8, 9, 10, 11, 12, 13, 14, 15, 16, 17, 18, 19, 20, 21, 22, 23, 24, 25, 26, 27, 28, 29, 30 μg/mL expression as determined by the method disclosed herein.

In some embodiments, an anti-TL1A antibody comprises antibody A. As used herein, antibody A comprises the CDRs of antibody A in Table 20. In some cases, antibody A comprises a heavy chain framework comprising SEQ ID NO: 301 (Embodiment X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody A comprises a light chain framework comprising SEQ ID NO: 303 (Embodiment EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK), wherein X10=L or P and X11=L or W. In some cases, antibody A comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A comprises a constant region comprising reduced ADCC and/or CDC as compared to IgG1. For instance, antibody A comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody A comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody A comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody A is expressed from FreeStyle 293-F (e.g., ThermoFisher Scientific #R79007) cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody A is expressed from FreeStyle 293-F cells at an expression level of between about 2 μg/mL to about 60 μg/mL. In some cases, antibody A is expressed from FreeStyle 293-F cells at an expression level of between about 10 μg/mL to about 60 μg/mL. In some cases, antibody A comprises a viscosity less than about 30 mPa-s. In some cases, antibody A comprises a viscosity from about 4 mPa-s to about 30 mPa-s, or about 4, 5, 6, 7, 8, 9, 10. 11, 12, 13, 14, 15, 16, 17, 18, 19, 20, 21, 22, 23, 24, 25, 26, 27, 28, 29, 30 mPa-s. In some cases, antibody A is formulated in a solution having a concentration of about 10 mg/ml to about 170 mg/ml, with a viscosity less than about 30 mPa-s. In some cases, the formulation has a pH of about 5 to about 7.5.

In some embodiments, an anti-TL1A antibody comprises antibody B. As used herein, antibody B comprises the CDRs of antibody B in Table 20. In some cases, antibody B comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody B comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody B comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B comprises a constant region comprising reduced ADCC and/or CDC as compared to IgG1. For instance, antibody B comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody B comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody B comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody B is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody B is expressed from FreeStyle 293-F cells at an expression level of between about 2 μg/mL to about 60 μg/mL. In some cases, antibody B is expressed from FreeStyle 293-F cells at an expression level of between about 10 μg/mL to about 60 μg/mL.

In some embodiments, an anti-TL1A antibody comprises antibody C. As used herein, antibody C comprises the CDRs of antibody C in Table 20. In some cases, antibody C comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody C comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody C comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C comprises a constant region comprising reduced ADCC and/or CDC as compared to IgG1. For instance, antibody C comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, ( ) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody C comprises a constant region comprising at least about 85%, 87%, 88%, 89%, 90%, 91%, 92%, 93%, 94%, 95%, 96%, 97% 98% 99%, or 100% identity to SEQ ID NO: 323. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody C comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody C comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody C is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody C is expressed from FreeStyle 293-F cells at an expression level of between about 2 μg/mL to about 60 μg/mL. In some cases, antibody C is expressed from FreeStyle 293-F cells at an expression level of between about 10 μg/mL to about 60 μg/mL.

In some embodiments, an anti-TL1A antibody comprises antibody D. As used herein, antibody D comprises the CDRs of antibody D in Table 20. In some cases, antibody D comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R orK, X3=A or R, X4=M or I, X5=V or A, X6=M or I, X7=R or T, X8=V or A, and X9=M or L. In some cases, antibody D comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody D comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D comprises a constant region comprising reduced ADCC and/or CDC as compared to IgG1. For instance, antibody D comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody D comprises a constant region comprising at least about 85%, 87%, 88%, 89%, 90%, 91%, 92%, 93%, 94%, 95%, 96%, 97% 98% 99%, or 100% identity to SEQ ID NO: 331. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody D comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody D comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody D is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody D is expressed from FreeStyle 293-F cells at an expression level of between about 2 μg/mL to about 60 μg/mL. In some cases, antibody D is expressed from FreeStyle 293-F cells at an expression level of between about 10 μg/mL to about 60 μg/mL.

In some embodiments, an anti-TL1A antibody comprises antibody E. As used herein, antibody E comprises the CDRs of antibody E in Table 20. In some cases, antibody E comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody E comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody E comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E comprises a constant region comprising reduced ADCC and/or CDC as compared to IgG1. For instance, antibody E comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody E comprises a constant region comprising at least about 85%, 87%, 88%, 89%, 90%, 91%, 92%, 93%, 94%, 95%, 96%, 97% 98% 99%, or 100% identity to SEQ ID NO: 339. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody E comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody E comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody E is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody E is expressed from FreeStyle 293-F cells at an expression level of between about 2 μg/mL to about 60 μg/mL. In some cases, antibody E is expressed from FreeStyle 293-F cells at an expression level of between about 10 μg/mL to about 60 μg/mL.

In some embodiments, an anti-TL1A antibody comprises antibody F. As used herein, antibody F comprises the CDRs of antibody F in Table 20. In some cases, antibody F comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody F comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody F comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F comprises a constant region comprising reduced ADCC and/or CDC as compared to IgG1. For instance, antibody F comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody F comprises a constant region comprising at least about 85%, 87%, 88%, 89%, 90%, 91%, 92% 93%, 94%, 95%, 96%, 97%, 98%, 99%, or 100% identity to SEQ ID NO: 347. In some cases, antibody F comprises a constant region comprising at least about 85%, 87%, 88%, 89%, 9%, 91%, 92%, 93%, 94%, 95%, 96%, 97%, 98%, 99%, or 100% identity to SEQ ID NO: 348. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody F comprises a constant region comprising at least about 85%, 87%, 88%, 89%, 90%, 91%, 92%, 930, 94%, 95%, 96%, 97%, 98%, 99%, or 100% identity to SEQ ID NO: 359. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody F comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody F comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody F is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody F is expressed from FreeStyle 293-F cells at an expression level of between about 2 μg/mL to about 60 μg/mL. In some cases, antibody F is expressed from FreeStyle 293-F cells at an expression level of between about 10 μg/mL to about 60 μg/mL.

In some embodiments, an anti-TL1A antibody comprises antibody G. As used herein, antibody G comprises the CDRs of antibody G in Table 20. In some cases, antibody G comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody G comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody G comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G comprises a constant region comprising reduced ADCC and/or CDC as compared to IgG1. For instance, antibody G comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (j) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody G comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody G comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody G is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody G is expressed from FreeStyle 293-F cells at an expression level of between about 2 μg/mL to about 60 μg/mL. In some cases, antibody G is expressed from FreeStyle 293-F cells at an expression level of between about 10 μg/mL to about 60 μg/mL.

In some embodiments, an anti-TL1A antibody comprises antibody H. As used herein, antibody H comprises the CDRs of antibody H in Table 20. In some cases, antibody H comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody H comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody H comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H comprises a constant region comprising reduced ADCC and/or CDC as compared to IgG1. For instance, antibody H comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody H comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody H comprises at least about 80%, 81%, 82%, 83%, 84%, 85%, 85%, 87%, 88%, 89%, 90%, 91% 92%, 93%, 94%, 95% 96% 97%, 98%, 99%, or 100% monomeric fraction as measured by the size exclusion method described in Example 2. In some cases, antibody H is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody H is expressed from FreeStyle 293-F cells at an expression level of between about 2 μg/mL to about 60 μg/mL. In some cases, antibody H is expressed from FreeStyle 293-F cells at an expression level of between about 10 μg/mL to about 60 μg/mL.

In some embodiments, an anti-TL1A antibody comprises antibody I. As used herein, antibody I comprises the CDRs of antibody I in Table 20. In some cases, antibody I comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody I comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody I comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody I comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody I comprises a constant region comprising reduced ADCC and/or CDC as compared to IgG1. For instance, antibody I comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P³31A or P331S, or (nnn) any combination of (a)-(uu), per Kabat numbering. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody I comprises a constant region comprising at least about 85%, 87%, 88%, 89% 90%, 91%, 92%, 93%, 94%, 95%, 96%, 97%, 98%, 99%, or 100% identity to SEQ ID NO: 352. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody I comprises a constant region comprising at least about 85%, 87%, 88%, 89%, 90%, 91%, 92%, 93%, 94%, 95%, 96%, 97% 98% 99%, or 100% identity to SEQ ID NO: 358. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody I comprises a constant region comprising at least about 85%, 87%, 88%, 9%, 90%, 91%, 92%, 93% 94%, 95%, 96%, 97%, 98%, 99%, or 100% identity to SEQ ID NO: 361. In some cases, antibody I comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody I comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody I is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody I is expressed from FreeStyle 293-F cells at an expression level of between about 2 μg/mL to about 60 μg/mL. In some cases, antibody I is expressed from FreeStyle 293-F cells at an expression level of between about 10 μg/mL to about 60 μg/mL.

In some embodiments, an anti-TL1A antibody comprises antibody A2. As used herein, antibody A2 comprises the CDRs of antibody A2 in Table 20. In some cases, antibody A2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody A2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody A2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody A2 comprises a constant region comprising reduced ADCC and/or CDC as compared to IgG1. For instance, antibody A2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody A2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody A2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody A2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody A2 is expressed from FreeStyle 293-F cells at an expression level of between about 2 μg/mL to about 60 μg/mL. In some cases, antibody A2 is expressed from FreeStyle 293-F cells at an expression level of between about 10 μg/mL to about 60 μg/mL.

In some embodiments, an anti-TL1A antibody comprises antibody B2. As used herein, antibody B2 comprises the CDRs of antibody B2 in Table 20. In some cases, antibody B2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody B2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody B2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody B2 comprises a constant region comprising reduced ADCC and/or CDC as compared to IgG1. For instance, antibody B2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody B2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody B2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody B2 is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody B2 is expressed from FreeStyle 293-F cells at an expression level of between about 2 μg/mL to about 60 μg/mL. In some cases, antibody B2 is expressed from FreeStyle 293-F cells at an expression level of between about 10 μg/mL to about 60 μg/mL.

In some embodiments, an anti-TL1A antibody comprises antibody C2. As used herein, antibody C2 comprises the CDRs of antibody C2 in Table 20. In some cases, antibody C2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody C2 comprises a light chain framework comprising SEQ ID NO: 303 (EIVLTQSPGTLSLSPGERATLSC[LCDR1]WYQQKPGQAPRX10X11Y[LCDR2]GIPDRFS GSGSGTDFTLTISRLEPEDFAVYYC[LCDR3]FGGGTKLEIK), wherein X10=L or P and X11=L or W. In some cases, antibody C2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody C2 comprises a constant region comprising reduced ADCC and/or CDC as compared to IgG1. For instance, antibody C2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody C2 comprises a constant region comprising at least about 85%, 87%, 88%, 89%, 90%, 91%, 92%, 93%, 94%, 95%, 96%, 97% 98% 99%, or 100% identity to SEQ ID NO: 322. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody C2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody C2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody C2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody C2 is expressed from FreeStyle 293-F cells at an expression level of between about 2 μg/mL to about 60 μg/mL. In some cases, antibody C2 is expressed from FreeStyle 293-F cells at an expression level of between about 10 μg/mL to about 60 μg/mL.

In some embodiments, an anti-TL1A antibody comprises antibody D2. As used herein, antibody D2 comprises the CDRs of antibody D2 in Table 20. In some cases, antibody D2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody D2 comprises a light chain framework comprising SEQ ID NO: 303 (EIVLTQSPGTLSLSPGERATLSC[LCDR1]WYQQKPGQAPRXIOX11IY[LCDR2]GIPDRFS GSGSGTDFTLTISRLEPEDFAVYYC[LCDR3]FGGGTKLEIK), wherein X10=L or P and X11=L or W. In some cases, antibody D2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody D2 comprises a constant region comprising reduced ADCC and/or CDC as compared to IgG1. For instance, antibody D2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody D2 comprises a constant region comprising at least about 85%, 87%, 88%, 89%, 90%, 91%, 92%, 93%, 94%, 95%, 96%, 97% 98% 99%, or 100% identity to SEQ ID NO: 330. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody D2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody D2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody D2 is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody D2 is expressed from FreeStyle 293-F cells at an expression level of between about 2 μg/mL to about 60 μg/mL. In some cases, antibody D2 is expressed from FreeStyle 293-F cells at an expression level of between about 10 μg/mL to about 60 μg/mL.

In some embodiments, an anti-TL1A antibody comprises antibody E2. As used herein, antibody E2 comprises the CDRs of antibody E2 in Table 20. In some cases, antibody E2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody E2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody E2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody E2 comprises a constant region comprising reduced ADCC and/or CDC as compared to IgG1. For instance, antibody E2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody E2 comprises a constant region comprising at least about 85%, 87%, 88%, 89%, 90%, 91%, 92%, 93%, 94%, 95%, 96%, 97% 98% 99%, or 100% identity to SEQ ID NO: 338. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody E2 comprises a constant region comprising at least about 85%, 87%, 88%, 89%, 90%, 91%, 92%, 93%, 94%, 95%, 96%, 97% 98% 99%, or 100% identity to SEQ ID NO: 354. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody E2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody E2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody E2 is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody E2 is expressed from FreeStyle 293-F cells at an expression level of between about 2 μg/mL to about 60 μg/mL. In some cases, antibody E2 is expressed from FreeStyle 293-F cells at an expression level of between about 10 μg/mL to about 60 μg/mL.

In some embodiments, an anti-TL1A antibody comprises antibody F2. As used herein, antibody F2 comprises the CDRs of antibody F2 in Table 20. In some cases, antibody F2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody F2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody F2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody F2 comprises a constant region comprising reduced ADCC and/or CDC as compared to IgG1. For instance, antibody F2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody F2 comprises a constant region comprising at least about 85%, 87%, 88%, 89%, 90%, 91%, 92%, 93%, 94%, 95%, 96% 97%, 98%, 99%, or 100% identity to SEQ ID NO: 323. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody F2 comprises a constant region comprising at least about 85%, 87%, 88%, 89%, 90%, 91%, 92%, 93%, 94%, 95%, 96%, 97% 98% 99%, or 100% identity to SEQ ID NO: 346. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody F2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody F2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody F2 is expressed from FreeStyle 293-F cells at an expression level of about or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody F2 is expressed from FreeStyle 293-F cells at an expression level of between about 2 μg/mL to about 60 μg/mL. In some cases, antibody F2 is expressed from FreeStyle 293-F cells at an expression level of between about 10 μg/mL to about 60 μg/mL.

In some embodiments, an anti-TL1A antibody comprises antibody G2. As used herein, antibody G2 comprises the CDRs of antibody G2 in Table 20. In some cases, antibody G2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody G2 comprises a light chain framework comprising SEQ ID NO: 303 (EIVLTQSPGTLSLSPGERATLSC[LCDR1]WYQQKPGQAPRXIOX11IY[LCDR2]GIPDRFS GSGSGTDFTLTISRLEPEDFAVYYC[LCDR3]FGGGTKLEIK), wherein X10=L or P and X11=L or W. In some cases, antibody G2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody G2 comprises a constant region comprising reduced ADCC and/or CDC as compared to IgG1. For instance, antibody G2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 323. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody G2 comprises a constant region comprising at least about 85%, 87%, 88%, 89%, 90%, 91%, 92%, 93%, 94%, 95%, 96%, 97% 98% 99%, or 100% identity to SEQ ID NO: 354. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody G2 comprises a constant region comprising at least about 85%, 87%, 88%, 89%, 90%, 91%, 92%, 93%, 94%, 95%, 96%, 97%, 98%, 99%, or 100% identity to SEQ ID NO: 362. In some cases, antibody G2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody G2 is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody G2 is expressed from FreeStyle 293-F cells at an expression level of between about 2 μg/mL to about 60 μg/mL. In some cases, antibody G2 is expressed from FreeStyle 293-F cells at an expression level of between about 10 μg/mL to about 60 μg/mL.

In some embodiments, an anti-TL1A antibody comprises antibody H2. As used herein, antibody H2 comprises the CDRs of antibody H2 in Table 20. In some cases, antibody H2 comprises a heavy chain framework comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), wherein X1=Q or E, X2=R or K, X3=A or R, X4=M or I, X5=V or A, X6=M or I, X7=R or T, X8=V or A, and X9=M or L. In some cases, antibody H2 comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein X10=L or P and X11=L or W. In some cases, antibody H2 comprises a heavy chain variable region comprising human IGHV1-46*02 framework or a modified human IGHV1-46*02 framework, wherein the modified human IGHV1-46*02 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H2 comprises a light chain variable region comprising human IGKV3-20 framework or a modified human IGKV3-20 framework, wherein the modified human IGKV3-20 framework has less than or equal to about 1, 2, 3, 4, 5, 6, 7, 8, 9, 10, 11, 12, 13, 14, 15, 16, 17, 18, 19, or 20 amino acid substitutions in the framework. In some cases, antibody H2 comprises a constant region comprising reduced ADCC and/or CDC as compared to IgG1. For instance, antibody H2 comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 320. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 321. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 322. In some cases, antibody H2 comprises a constant region comprising at least about 85%, 87%, 88%, 89%, 90%, 91%, 92%, 93%, 94%, 95%, 96% 97%, 98%, 99%, or 100% identity to SEQ ID NO: 323. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 324. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 325. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 326. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 327. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 328. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 329. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 330. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 331. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 332. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 333. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 334. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 335. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 336. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 337. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 338. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 339. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 340. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 341. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 342. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 343. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 344. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 345. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 346. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 347. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 348. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 349. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 350. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 351. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 352. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 353. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 354. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 355. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 356. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 357. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 358. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 359. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 360. In some cases, antibody H2 comprises a constant region comprising at least about 85%, 87%, 88%, 89%, 90%, 91%, 92%, 93%, 94%, 95%, 96%, 97%, 98%, 99%, or 100% identity to SEQ ID NO: 361. In some cases, antibody H2 comprises a constant region comprising at least about 85%, 87%, 88%, 89%, 90%, 91%, 92%, 93%, 94%, 95%, 96%, 97% 98% 99%, or 100% identity to SEQ ID NO: 362. In some cases, antibody H2 comprises at least about 80%, 81%, 82%, 83%, 84%, 85%, 85%, 87%, 88%, 89%, 90%, 91%, 92%, 93%, 94%, 95%, 96%, 97%, 98%, 99%, or 100% monomeric fraction as measured by the size exclusion method described in Example 2. In some cases, antibody H2 is expressed from FreeStyle 293-F cells at an expression level of about or at least about 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 57, 58, 59 or 60 μg/mL as determined by the method described in Example 2. In some cases, antibody H2 is expressed from FreeStyle 293-F cells at an expression level of between about 2 μg/mL to about 60 μg/mL. In some cases, antibody H2 is expressed from FreeStyle 293-F cells at an expression level of between about 10 μg/mL to about 60 μg/mL.

The TL1A antibodies described herein bind to specific regions or epitopes of human TL1A demonstrated herein as useful to inhibit interferon gamma secretion from T lymphocytes. Various embodiments provide for an anti-TL1A antibody that binds to the same region of a TL1A protein or portion thereof as a reference antibody such as the anti-TL1A antibodies described herein. In some embodiments, the reference antibody comprises antibody A, B, C, D, E, F, G, H, A2, B2, C2, D2, E2, F2, G2, or H2, or a combination thereof. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 104, and a light chain comprising a sequence at least about 90%, 92%, 93%, 94%, 95%, 96%, 97%, 98%, 99% or 100% identical to SEQ ID NO: 201. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 107, and a light chain comprising a sequence at least about 90%, 92%, 93%, 94%, 95%, 96%, 97%, 98%, 99% or 100% identical to SEQ ID NO: 201.

Non-limiting methods for determining whether an anti-TL1A antibody (i.e. test antibody) binds to the same region of a TL1A protein or portion thereof as an antibody described herein are provided. An exemplary embodiment comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in the examples.

In certain embodiments, disclosed herein are antibodies that compete for binding TL1A with the antibodies described herein. In certain embodiments, disclosed herein are antibodies that bind a discrete epitope that overlaps with an epitope of TL1A bound by an antibody described herein. In certain embodiments, disclosed herein are antibodies that bind the same epitope of TL1A, overlap with an epitope of TL1A by one or more amino acid residues, or that compete for binding to an epitope of TL1A with an antibody or fragment thereof that comprises a heavy chain variable region comprising the amino acid sequence of SEQ ID NO: 104; and a light chain variable region comprising the amino acid of SEQ ID NO: 201. In certain embodiments, disclosed herein are antibodies that bind the same epitope of TL1A, overlap with an epitope of TL1A by one or more amino acid residues, or that compete for binding to an epitope of TL1A with an antibody or fragment thereof that comprises a heavy chain variable region comprising the amino acid sequence of SEQ ID NO: 107; and a light chain variable region comprising the amino acid of SEQ ID NO: 201.

### 5.4.1.5 Additional Antibody Embodiments

In one aspect, provided herein, is an antibody or antigen binding fragment thereof that binds to TL1A, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework. In some embodiments, the amino acid modification comprises: (a) a modification at amino acid position 47 in the heavy chain variable region; (b) a modification at amino acid position 45 in the heavy chain variable region; (c) a modification at amino acid position 55 in the heavy chain variable region; (d) a modification at amino acid position 78 in the heavy chain variable region; (e) a modification at amino acid position 80 in the heavy chain variable region; (f) a modification at amino acid position 82 in the heavy chain variable region; (g) a modification at amino acid position 89 in the heavy chain variable region; or (h) a modification at amino acid position 91 in the heavy chain variable region; per Aho or Kabat numbering; or a combination of two or more modifications selected from (a) to (h). In some embodiments, (a) the amino acid modification is at position 47 in the heavy chain variable region, and the amino acid at position 47 is R, N, D, C, Q, E, G, H, I, L, K, M, F, P, S, T, W, Y, or V; (b) the amino acid modification is at position 45 in the heavy chain variable region, and the amino acid at position 45 is A, N, D, C, Q, E, G, H, I, L, K, M, F, P, S, T, W, Y, or V; (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N, D, C, Q, E, G, H, I, L, K, M, F, P, S, T, W, or Y; (e) the amino acid modification is at position 80 in the heavy chain variable region, and the amino acid at position 80 is A, R, N, D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V; (g) the amino acid modification is at position 89 in the heavy chain variable region, and the amino acid at position 89 is A, R, N, D, C, Q, E, G, H, I, L, K, M, F, P, S, T, W, or Y; or (h) the amino acid modification is at position 91 in the heavy chain variable region, and the amino acid at position 91 is A, R, N, D, C, Q, E, G, H, I, L, K, F, P, S, T, W, Y, or V; or a combination of two or more modifications selected from (a) to (h). In some embodiments, the amino acid modifications comprise one or more of: A47R, R45K, M55I, V78A, M80I, R82T, V89A, M91L in the heavy chain variable region, per Aho or Kabat numbering. In some embodiments, the amino acid modification comprises: (a) a modification at amino acid position 54 in the light chain variable region; and/or (b) a modification at amino acid position 55 in the light chain variable region; per Aho or Kabat numbering. In some embodiments, (a) the amino acid modification is at position 54 of the light chain variable region, and the amino acid at position 54 is A, R, N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V. In some embodiments, the amino acid modifications comprise L54P and/or L55W in the light chain variable region, per Aho or Kabat numbering. In some embodiments, the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15. In some embodiments, the antibody or antigen binding fragment comprises a heavy chain CDR1 as set forth by SEQ ID NO: 1, a heavy chain CDR2 as set forth by SEQ ID NO: 2, a heavy chain CDR3 as set forth by SEQ ID NO: 6, a light chain CDR1 as set forth by SEQ ID NO: 10, a light chain CDR2 as set forth by SEQ ID NO: 11, and a light chain CDR3 as set forth by SEQ ID NO: 12. In some embodiments, the antibody or antigen binding fragment comprises comprising a heavy chain framework (FR) 1 as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof. In some embodiments, the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of two or more selected from (a)-(uu), per Kabat numbering. In some embodiments, the antibody or antigen binding fragment comprises a human IgG4 Fc region. In some embodiments, the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362. In some embodiments, the antibody or antigen binding fragment comprises at least about 80% monomeric fraction as determined by size exclusion chromatography. In some embodiments, the antibody or antigen binding fragment expresses at least about 20 μg/ml total antibody, optionally about 20 μg/ml and 70 μg/mL total antibody.

In another aspect, provided herein is an antibody or antigen binding fragment thereof that binds to tumor necrosis factor-like protein 1A (TL1A), comprising a heavy chain variable domain comprising an amino acid sequence at least 96% identical to SEQ ID NO: 104, and a light chain variable domain comprising an amino acid sequence at least 97% identical to SEQ ID NO: 201. In some embodiments, the heavy chain variable domain comprises an amino acid sequence at least 97% identical to SEQ ID NO: 104. In some embodiments, the heavy chain variable domain comprises an amino acid sequence at least 98% identical to SEQ ID NO: 104. In some embodiments, the heavy chain variable domain comprises an amino acid sequence at least 99% identical to SEQ ID NO: 104. In some embodiments, the heavy chain variable domain comprises SEQ ID NO: 104. In some embodiments, the light chain variable domain comprises an amino acid sequence at least 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

In another aspect, provided herein is an antibody or antigen binding fragment thereof that binds to tumor necrosis factor-like protein 1A (TL1A), comprising a heavy chain variable domain comprising an amino acid sequence at least about 99% identical to any one of SEQ ID NOS: 101-135, and a light chain variable domain comprising an amino acid sequence at least about 99% identical to any one of SEQ ID NOS: 201-206.

In some embodiments, antibodies or antigen binding fragments described herein comprise a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of two or more selected from (a)-(uu), per Kabat numbering. In some embodiments, antibodies or antigen binding fragments described herein comprise a human IgG4 Fc region. In some embodiments, antibodies or antigen binding fragments described herein comprise a Fc region comprising a sequence at least about 90%, 91%, 92%, 93%, 94%, 95%, 96%, 97%, 98%, 99%, or 100% identical to any one of SEQ ID NOS: 320-362. In some embodiments, antibodies or antigen binding fragments described herein comprise at least about 80% monomeric fraction as determined by size exclusion chromatography. In some embodiments, antibodies or antigen binding fragments described herein express at least about 20 μg/ml total antibody, optionally about 20 μg/ml and 70 μg/mL total antibody.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; (f) an LCDR3 comprising an amino acid sequence set forth by any one of SEQ ID NOS: 12-15; and a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1. In some embodiments, the human IgG1 comprises SEQ ID NO: 320. In some embodiments, the ADCC function of the Fc region comprising reduced ADCC is at least about 50% reduced as compared to human IgG1. In some embodiments, the CDC function of the Fc region comprising reduced CDC is at least about 50% reduced as compared to human IgG1. In some embodiments, the Fc region comprises a human IgG1 comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some embodiments, the antibody or antigen binding fragment comprises a (i) human IgG4 Fc region or (ii) a human IgG4 Fc region comprising (a) S228P, (b) S228P and L235E, or (c) S228P, F234A, and L235A, per Kabat numbering. In some embodiments, the antibody or antigen binding fragment comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). In some embodiments, the Fc region comprises a human IgG1 with a substitution selected from 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering. In some embodiments, the antibody or antigen binding fragment comprises a sequence at least about 90%, 91%, 92%, 93%, 94%, 95%, 96%, 97%, 98%, 99%, or 100% identical to any one of SEQ ID NOS: 320-362. In some embodiments, the HCDR1 comprises SEQ ID NO: 1, HCDR2 comprises any one of SEQ ID NOS: 2-5, HCDR3 comprises any one of SEQ ID NOS: 6-9, LCDR1 comprises SEQ ID NO: 10, LCDR2 comprises SEQ ID NO: 11, and LCDR3 comprises any one of SEQ ID NOS: 12-15. In some embodiments, the Fc region comprises any one of SEQ ID NOs: 401-413 or a sequence at least about 90%, 91%, 92%, 93%, 94%, 95%, 96%, 97%, 98%, or 99% identical to any one of SEQ ID NOs: 401-413. In some embodiments, the heavy chain comprises any one of SEQ ID NOs: 501-513 or a sequence at least about 90%, 91%, 92%, 93%, 94%, 95%, 96%, 97%, 98%, or 99% identical to any one of SEQ ID NOs: 501-513. In some embodiments, the light chain comprises any one of SEQ ID NO: 514 or a sequence at least about 90%, 91%, 92%, 93%, 94%, 95%, 96%, 97%, 98%, or 99% identical to any one of SEQ ID NO: 514. In some embodiments, the HCDR2 comprises SEQ ID NO: 2, the HCDR3 comprises SEQ ID NO: 6, and the LCDR3 comprises SEQ ID NO: 12, and wherein the Fc region comprises any one of SEQ ID NOs: 401-413 or a sequence at least about 90%, 91%, 92%, 93%, 94%, 95%, 96%, 97%, 98%, or 99% identical to any one of SEQ ID NOs: 401-413. In some embodiments, the antibody or antigen binding fragment comprises at least about 80% monomeric fraction as determined by size exclusion chromatography. In some embodiments, the antibody or antigen binding fragment expresses at least about 20 μg/ml total antibody, optionally about 20 μg/ml and 70 μg/mL total antibody.

Further provided are methods of treating fibrosis or an intestinal inflammatory condition, disease, or disorder in a subject in need thereof, the method comprising administering to the subject an antibody or antigen binding fragment of any antibody or antigen binding fragment disclosed herein. In some embodiments, the subject has fibrosis. In some embodiments, the subject has an intestinal inflammatory condition, disease, or disorder. In some embodiments, the intestinal inflammatory condition, disease, or disorder comprises inflammatory bowel disease (IBD). In some embodiments, the IBD comprises Crohn's Disease. In some embodiments, the IBD comprises ulcerative colitis.

In some embodiments, antibodies or antigen binding fragments described herein comprise are present in a liquid composition a concentration of the antibody or antigen binding fragment of 10 mg/ml to 170 mg/ml. In some embodiments, the antibody or antigen binding fragment thereof is at a concentration of 10 mg/ml to 170 mg/ml. In some embodiments, the viscosity is from about 4 to about 30 mPa-s (millipascal-second (mPa s)). In some embodiments, the viscosity is from about 4 to about 10 mPa-s (millipascal-second (mPa-s)).

In another aspect, provided herein, is an antibody or antigen binding fragment thereof that binds to TL1A, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework. In some embodiments, the heavy chain variable framework region and the light chain variable framework region collectively comprise 13, 12, 11, 10, 9, 8, 7, 6, 5, 4, 3, 2, 1, or no amino acid modifications from the human IGHV1-46*02 framework and the human IGKV3-20 framework. In some embodiments, the amino acid modification comprises a modification at amino acid position 1 in the heavy chain variable region, per Aho or Kabat numbering. In some embodiments, the amino acid at position 1 comprises A, R, N, D, C, E, G, H, I, L, K, M, F, P, S, T, W, Y, or V. In some embodiments, the amino acid at position 1 comprises a hydrophobic amino acid, a hydrophilic amino acid, or an amphipathic amino acid. In some embodiments, the amino acid at position 1 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 1 comprises E. In some embodiments, the amino acid modification comprises a modification at amino acid position 45 in the heavy chain variable region, per Aho or Kabat numbering. In some embodiments, the amino acid at position 45 comprises A, N, D, C, Q, E, G, H, I, L, K, M, F, P, S, T, W, Y, or V. In some embodiments, the amino acid at position 45 comprises a hydrophobic amino acid, a hydrophilic amino acid, or an amphipathic amino acid. In some embodiments, the amino acid at position 45 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 45 comprises K. In some embodiments, the amino acid modification comprises a modification at amino acid position 47 in the heavy chain variable region, per Aho or Kabat numbering. In some embodiments, the amino acid at position 47 comprises R, N, D, C, Q, E, G, H, I, L, K, M, F, P, S, T, W, Y, or V. In some embodiments, the amino acid at position 47 comprises a hydrophobic amino acid, a hydrophilic amino acid, or an amphipathic amino acid. In some embodiments, the amino acid at position 47 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 47 comprises R. In some embodiments, the amino acid modification comprises a modification at amino acid position 55 in the heavy chain variable region, per Aho or Kabat numbering. In some embodiments, the amino acid at position 55 comprises A, R, N, D, C, Q, E, G, H, I, L, K, F, P, S, T, W, Y, or V. In some embodiments, the amino acid at position 55 comprises a hydrophobic amino acid, a hydrophilic amino acid, or an amphipathic amino acid. In some embodiments, the amino acid at position 55 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 55 comprises I. In some embodiments, the amino acid modification comprises a modification at amino acid position 78 in the heavy chain variable region, per Aho or Kabat numbering. In some embodiments, the amino acid at position 78 comprises A, R, N, D, C, Q, E, G, H, I, L, K, M, F, P, S, T, W, or Y. In some embodiments, the amino acid at position 78 comprises a hydrophobic amino acid, a hydrophilic amino acid, or an amphipathic amino acid. In some embodiments, the amino acid at position 78 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 78 comprises A. In some embodiments, the amino acid modification comprises a modification at amino acid position 80 in the heavy chain variable region, per Aho or Kabat numbering. In some embodiments, the amino acid at position 80 comprises A, R, N, D, C, Q, E, G, H, I, L, K, F, P, S, T, W, Y, or V. In some embodiments, the amino acid at position 80 comprises a hydrophobic amino acid, a hydrophilic amino acid, or an amphipathic amino acid. In some embodiments, the amino acid at position 80 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 80 comprises I. In some embodiments, the amino acid modification comprises a modification at amino acid position 82 in the heavy chain variable region, per Aho or Kabat numbering. In some embodiments, the amino acid at position 82 comprises A, N, D, C, Q, E, G, H, I, L, K, M, F, P, S, T, W, Y, or V. In some embodiments, the amino acid at position 82 comprises a hydrophobic amino acid, a hydrophilic amino acid, or an amphipathic amino acid. In some embodiments, the amino acid at position 82 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 82 comprises T. In some embodiments, the amino acid modification comprises a modification at amino acid position 89 in the heavy chain variable region, per Aho or Kabat numbering. In some embodiments, the amino acid at position 89 comprises A, R, N, D, C, Q, E, G, H, I, L, K, M, F, P, S, T, W, or Y. In some embodiments, the amino acid at position 89 comprises a hydrophobic amino acid, a hydrophilic amino acid, or an amphipathic amino acid. In some embodiments, the amino acid at position 89 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 89 comprises A. In some embodiments, the amino acid modification comprises a modification at amino acid position 91 in the heavy chain variable region, per Aho or Kabat numbering. In some embodiments, the amino acid at position 91 comprises A, R, N, D, C, Q, E, G, H, I, L, K, F, P, S, T, W, Y, or V. In some embodiments, the amino acid at position 91 comprises a hydrophobic amino acid, a hydrophilic amino acid, or an amphipathic amino acid. In some embodiments, the amino acid at position 91 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 91 comprises L.

In some embodiments, the amino acid modifications comprise one or more of Q1E, R45K, A47R, M55I, V78A, M80I, R82T, V89A, M91L in the heavy chain variable region, per Aho or Kabat numbering. In some embodiments, the amino acid modifications comprise Q1E. In some embodiments, the amino acid modifications comprise R45K. In some embodiments, the amino acid modifications comprise A47R. In some embodiments, the amino acid modifications comprise M55I. In some embodiments, the amino acid modifications comprise V78A. In some embodiments, the amino acid modifications comprise M80I. In some embodiments, the amino acid modifications comprise R82T. In some embodiments, the amino acid modifications comprise V89A. In some embodiments, the amino acid modifications comprise M91L.

In some embodiments, the amino acid modification comprises a modification at amino acid position 54 in the light chain variable region, per Aho or Kabat numbering. In some embodiments, the amino acid at position 54 comprises A, R, N, D, C, Q, E, G, H, I, K, M, F, P, S, T, W, Y, or V. In some embodiments, the amino acid at position 54 comprises a hydrophobic amino acid, a hydrophilic amino acid, or an amphipathic amino acid. In some embodiments, the amino acid at position 54 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 54 comprises P. In some embodiments, the amino acid modification comprises a modification at amino acid position 55 in the light chain variable region, per Aho or Kabat numbering. In some embodiments, the amino acid at position 55 comprises A, R, N, D, C, Q, E, G, H, I, K, M, F, P, S, T, W, Y, or V. In some embodiments, the amino acid at position 55 comprises a hydrophobic amino acid, a hydrophilic amino acid, or an amphipathic amino acid. In some embodiments, the amino acid at position 55 comprises an aliphatic amino acid, an aromatic amino acid, an acidic amino acid, a basic amino acid, a hydroxylic amino acid, a sulfur-containing amino acid, or an amidic amino acid. In some embodiments, the amino acid at position 55 comprises W.

In some embodiments, the amino acid modifications comprise L54P and/or L55 W in the light chain variable region, per Aho or Kabat numbering. In some embodiments, the amino acid modifications comprise L54P. In some embodiments, the amino acid modifications comprise L55 W.

In some embodiments, the antibody or antigen binding fragment comprises a heavy chain CDR1 as set forth by SEQ ID NO: 1. In some embodiments, the antibody or antigen binding fragment comprises a heavy chain CDR2 as set forth by SEQ ID NO: 2. In some embodiments, the antibody or antigen binding fragment comprises a heavy chain CDR2 as set forth by SEQ ID NO: 3. In some embodiments, the antibody or antigen binding fragment comprises a heavy chain CDR2 as set forth by SEQ ID NO: 4. In some embodiments, the antibody or antigen binding fragment comprises a heavy chain CDR2 as set forth by SEQ ID NO: 5. In some embodiments, the antibody or antigen binding fragment comprises a heavy chain CDR3 as set forth by SEQ ID NO: 6. In some embodiments, the antibody or antigen binding fragment comprises a heavy chain CDR3 as set forth by SEQ ID NO: 7. In some embodiments, the antibody or antigen binding fragment comprises a heavy chain CDR3 as set forth by SEQ ID NO: 8. In some embodiments, the antibody or antigen binding fragment comprises a heavy chain CDR3 as set forth by SEQ ID NO: 9. In some embodiments, the antibody or antigen binding fragment comprises a light chain CDR1 as set forth by SEQ ID NO: 10. In some embodiments, the antibody or antigen binding fragment comprises a light chain CDR2 as set forth by SEQ ID NO: 11. In some embodiments, the antibody or antigen binding fragment comprises a light chain CDR3 as set forth by SEQ ID NO: 12. In some embodiments, the antibody or antigen binding fragment comprises a light chain CDR3 as set forth by SEQ ID NO: 13. In some embodiments, the antibody or antigen binding fragment comprises a light chain CDR3 as set forth by SEQ ID NO: 14. In some embodiments, the antibody or antigen binding fragment comprises a light chain CDR3 as set forth by SEQ ID NO: 15.

In some embodiments, the antibody or antigen binding fragment comprises a heavy chain FR1 as set forth by SEQ ID NO: 304. In some embodiments, the antibody or antigen binding fragment comprises a heavy chain FR2 as set forth by SEQ ID NO: 305. In some embodiments, the antibody or antigen binding fragment comprises a heavy chain FR2 as set forth by SEQ ID NO: 313. In some embodiments, the antibody or antigen binding fragment comprises a heavy chain FR3 as set forth by SEQ ID NO: 306. In some embodiments, the antibody or antigen binding fragment comprises a heavy chain FR3 as set forth by SEQ ID NO: 307. In some embodiments, the antibody or antigen binding fragment comprises a heavy chain FR3 as set forth by SEQ ID NO: 314. In some embodiments, the antibody or antigen binding fragment comprises a heavy chain FR3 as set forth by SEQ ID NO: 315. In some embodiments, the antibody or antigen binding fragment comprises a heavy chain FR4 as set forth by SEQ ID NO: 308. In some embodiments, the antibody or antigen binding fragment comprises a light chain FR1 as set forth by SEQ ID NO: 309. In some embodiments, the antibody or antigen binding fragment comprises a light chain FR2 as set forth by SEQ ID NO: 310. In some embodiments, the antibody or antigen binding fragment comprises a light chain FR3 as set forth by SEQ ID NO: 311. In some embodiments, the antibody or antigen binding fragment comprises a light chain FR4 as set forth by SEQ ID NO: 312.

In another aspect, provided herein is an antibody or antigen binding fragment thereof that binds to TL1A, comprising: (a) a heavy chain variable region comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS), and (b) a light chain variable region comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein each of X1-X11 is independently selected from A, R, N, D, C, Q, E, G, H, I, L, K, M, F, P, S, T, W, Y, or V.

In some embodiments, X1 comprises a hydrophobic amino acid, a hydrophilic amino acid, or an amphipathic amino acid. In some embodiments, X1 comprises an aliphatic amino acid, an aromatic amino acid, an acidic amino acid, a basic amino acid, a hydroxylic amino acid, a sulfur-containing amino acid, or an amidic amino acid. In some embodiments, X2 comprises a hydrophobic amino acid, a hydrophilic amino acid, or an amphipathic amino acid. In some embodiments, X2 comprises an aliphatic amino acid, an aromatic amino acid, an acidic amino acid, a basic amino acid, a hydroxylic amino acid, a sulfur-containing amino acid, or an amidic amino acid. In some embodiments, X3 comprises a hydrophobic amino acid, a hydrophilic amino acid, or an amphipathic amino acid. In some embodiments, X3 comprises an aliphatic amino acid, an aromatic amino acid, an acidic amino acid, a basic amino acid, a hydroxylic amino acid, a sulfur-containing amino acid, or an amidic amino acid. In some embodiments, X4 comprises a hydrophobic amino acid, a hydrophilic amino acid, or an amphipathic amino acid. In some embodiments, X4 comprises an aliphatic amino acid, an aromatic amino acid, an acidic amino acid, a basic amino acid, a hydroxylic amino acid, a sulfur-containing amino acid, or an amidic amino acid. In some embodiments, X5 comprises a hydrophobic amino acid, a hydrophilic amino acid, or an amphipathic amino acid. In some embodiments, X5 comprises an aliphatic amino acid, an aromatic amino acid, an acidic amino acid, a basic amino acid, a hydroxylic amino acid, a sulfur-containing amino acid, or an amidic amino acid. In some embodiments, X6 comprises a hydrophobic amino acid, a hydrophilic amino acid, or an amphipathic amino acid. In some embodiments, X6 comprises an aliphatic amino acid, an aromatic amino acid, an acidic amino acid, a basic amino acid, a hydroxylic amino acid, a sulfur-containing amino acid, or an amidic amino acid. In some embodiments, X7 comprises a hydrophobic amino acid, a hydrophilic amino acid, or an amphipathic amino acid. In some embodiments, X7 comprises an aliphatic amino acid, an aromatic amino acid, an acidic amino acid, a basic amino acid, a hydroxylic amino acid, a sulfur-containing amino acid, or an amidic amino acid. In some embodiments, X8 comprises a hydrophobic amino acid, a hydrophilic amino acid, or an amphipathic amino acid. In some embodiments, X8 comprises an aliphatic amino acid, an aromatic amino acid, an acidic amino acid, a basic amino acid, a hydroxylic amino acid, a sulfur-containing amino acid, or an amidic amino acid. In some embodiments, X9 comprises a hydrophobic amino acid, a hydrophilic amino acid, or an amphipathic amino acid. In some embodiments, X9 comprises an aliphatic amino acid, an aromatic amino acid, an acidic amino acid, a basic amino acid, a hydroxylic amino acid, a sulfur-containing amino acid, or an amidic amino acid. In some embodiments, X10 comprises a hydrophobic amino acid, a hydrophilic amino acid, or an amphipathic amino acid. In some embodiments, X10 comprises an aliphatic amino acid, an aromatic amino acid, an acidic amino acid, a basic amino acid, a hydroxylic amino acid, a sulfur-containing amino acid, or an amidic amino acid. In some embodiments, X11 comprises a hydrophobic amino acid, a hydrophilic amino acid, or an amphipathic amino acid. In some embodiments, X11 comprises an aliphatic amino acid, an aromatic amino acid, an acidic amino acid, a basic amino acid, a hydroxylic amino acid, a sulfur-containing amino acid, or an amidic amino acid.

In some embodiments, X1=Q or E, X2=R or K, X3=A or R, X4=M or I, X5=V or A, X6=M or I, X7=R or T, X8=V or A, X9=M or L, X10=L or P, and X11=L or W. In some embodiments, X1=Q. In some embodiments, X1=E. In some embodiments, X2=R. In some embodiments, X2=K. In some embodiments, X3=A. In some embodiments, X3=R. In some embodiments, X4=M. In some embodiments, X4=I. In some embodiments, X5=V. In some embodiments, X5=A. In some embodiments, X6=M. In some embodiments, X6=I. In some embodiments, X7=R. In some embodiments, X7=T. In some embodiments, X8=V. In some embodiments, X8=A. In some embodiments, X9=M. In some embodiments, X9=L. In some embodiments, X10=L. In some embodiments, X10=P. In some embodiments, X11=L. In some embodiments, X11=W.

In some embodiments, HCDR1 comprises SEQ ID NO: 1. In some embodiments, HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, the antibody or antigen binding fragment comprises a light chain CDR3 as set forth by SEQ ID NO: 15.

In another aspect, provided herein is an antibody or antigen binding fragment thereof that binds to TL1A, comprising: (a) a heavy chain variable region comprising SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS), and (b) a light chain variable region comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK), wherein each of X1-X11 is independently selected from A, R, N, D, C, Q, E, G, H, I, L, K, M, F, P, S, T, W, Y, or V.

In some embodiments, X1 comprises a hydrophobic amino acid, a hydrophilic amino acid, or an amphipathic amino acid. In some embodiments, X1 comprises an aliphatic amino acid, an aromatic amino acid, an acidic amino acid, a basic amino acid, a hydroxylic amino acid, a sulfur-containing amino acid, or an amidic amino acid. In some embodiments, X2 comprises a hydrophobic amino acid, a hydrophilic amino acid, or an amphipathic amino acid. In some embodiments, X2 comprises an aliphatic amino acid, an aromatic amino acid, an acidic amino acid, a basic amino acid, a hydroxylic amino acid, a sulfur-containing amino acid, or an amidic amino acid. In some embodiments, X3 comprises a hydrophobic amino acid, a hydrophilic amino acid, or an amphipathic amino acid. In some embodiments, X3 comprises an aliphatic amino acid, an aromatic amino acid, an acidic amino acid, a basic amino acid, a hydroxylic amino acid, a sulfur-containing amino acid, or an amidic amino acid. In some embodiments, X4 comprises a hydrophobic amino acid, a hydrophilic amino acid, or an amphipathic amino acid. In some embodiments, X4 comprises an aliphatic amino acid, an aromatic amino acid, an acidic amino acid, a basic amino acid, a hydroxylic amino acid, a sulfur-containing amino acid, or an amidic amino acid. In some embodiments, X5 comprises a hydrophobic amino acid, a hydrophilic amino acid, or an amphipathic amino acid. In some embodiments, X5 comprises an aliphatic amino acid, an aromatic amino acid, an acidic amino acid, a basic amino acid, a hydroxylic amino acid, a sulfur-containing amino acid, or an amidic amino acid. In some embodiments, X6 comprises a hydrophobic amino acid, a hydrophilic amino acid, or an amphipathic amino acid. In some embodiments, X6 comprises an aliphatic amino acid, an aromatic amino acid, an acidic amino acid, a basic amino acid, a hydroxylic amino acid, a sulfur-containing amino acid, or an amidic amino acid. In some embodiments, X7 comprises a hydrophobic amino acid, a hydrophilic amino acid, or an amphipathic amino acid. In some embodiments, X7 comprises an aliphatic amino acid, an aromatic amino acid, an acidic amino acid, a basic amino acid, a hydroxylic amino acid, a sulfur-containing amino acid, or an amidic amino acid. In some embodiments, X8 comprises a hydrophobic amino acid, a hydrophilic amino acid, or an amphipathic amino acid. In some embodiments, X8 comprises an aliphatic amino acid, an aromatic amino acid, an acidic amino acid, a basic amino acid, a hydroxylic amino acid, a sulfur-containing amino acid, or an amidic amino acid. In some embodiments, X9 comprises a hydrophobic amino acid, a hydrophilic amino acid, or an amphipathic amino acid. In some embodiments, X9 comprises an aliphatic amino acid, an aromatic amino acid, an acidic amino acid, a basic amino acid, a hydroxylic amino acid, a sulfur-containing amino acid, or an amidic amino acid. In some embodiments, X10 comprises a hydrophobic amino acid, a hydrophilic amino acid, or an amphipathic amino acid. In some embodiments, X10 comprises an aliphatic amino acid, an aromatic amino acid, an acidic amino acid, a basic amino acid, a hydroxylic amino acid, a sulfur-containing amino acid, or an amidic amino acid. In some embodiments, X11 comprises a hydrophobic amino acid, a hydrophilic amino acid, or an amphipathic amino acid. In some embodiments, X11 comprises an aliphatic amino acid, an aromatic amino acid, an acidic amino acid, a basic amino acid, a hydroxylic amino acid, a sulfur-containing amino acid, or an amidic amino acid.

In some embodiments, X1=Q or E, X2=R or K, X3=A or R, X4=M or I, X5=V or A, X6=M or I, X7=R or T, X8=V or A, X9=M or L, X10=L or P, and X11=L or W. In some embodiments, X1=Q. In some embodiments, X1=E. In some embodiments, X2=R. In some embodiments, X2=K. In some embodiments, X3=A. In some embodiments, X3=R. In some embodiments, X4=M. In some embodiments, X4=I. In some embodiments, X5=V. In some embodiments, X5=A. In some embodiments, X6=M. In some embodiments, X6=I. In some embodiments, X7=R. In some embodiments, X7=T. In some embodiments, X8=V. In some embodiments, X8=A. In some embodiments, X9=M. In some embodiments, X9=L. In some embodiments, X10=L. In some embodiments, X10=P. In some embodiments, X11=L. In some embodiments, X11=W.

In some embodiments, HCDR1 comprises SEQ ID NO: 1. In some embodiments, HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, LCDR3 comprises SEQ ID NO: 15.

Further provided herein is an antibody or antigen binding fragment thereof that binds to tumor necrosis factor-like protein 1A (TL1A), comprising a heavy chain variable domain comprising an amino acid sequence at least about 96% identical to SEQ ID NO: 104, and a light chain variable domain comprising an amino acid sequence at least about 97% identical to SEQ ID NO: 201. In some embodiments, the heavy chain variable domain comprises an amino acid sequence at least about 97% identical to SEQ ID NO: 104. In some embodiments, the heavy chain variable domain comprises an amino acid sequence at least about 98% identical to SEQ ID NO: 104. In some embodiments, the heavy chain variable domain comprises an amino acid sequence at least about 99% identical to SEQ ID NO: 104. In some embodiments, the heavy chain variable domain comprises SEQ ID NO: 104. In some embodiments, the light chain variable domain comprises an amino acid sequence at least about 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided herein is an antibody or antigen binding fragment thereof that binds to tumor necrosis factor-like protein 1A (TL1A), comprising a heavy chain variable domain comprising an amino acid sequence at least about 99% identical to SEQ ID NO: 107, and a light chain variable domain comprising an amino acid sequence at least about 97% identical to SEQ ID NO: 201. In some embodiments, the heavy chain variable domain comprises SEQ ID NO: 107. In some embodiments, the light chain variable domain comprises an amino acid sequence at least about 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided herein is an antibody or antigen binding fragment thereof that binds to tumor necrosis factor-like protein 1A (TL1A), comprising a heavy chain variable domain comprising SEQ ID NO: 101, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 102, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 103, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 104, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 105, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 103, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 106, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 109, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 109, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 203. Further provided herein is an antibody or antigen binding fragment thereof that binds to tumor necrosis factor-like protein 1A (TL1A), comprising a heavy chain variable domain comprising SEQ ID NO: 108, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 107, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 110, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 111, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 112, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 113, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 114, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 115, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 116, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 117, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 118, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 114, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 102, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 104, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 119, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 119, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 101, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 105, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 120, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 121, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 204. Further provided herein is an antibody or antigen binding fragment thereof that binds to tumor necrosis factor-like protein 1A (TL1A), comprising a heavy chain variable domain comprising SEQ ID NO: 123, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 202. Further provided herein is an antibody or antigen binding fragment thereof that binds to tumor necrosis factor-like protein 1A (TL1A), comprising a heavy chain variable domain comprising SEQ ID NO: 125, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 116, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 117, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 126, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 127, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 127, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 121, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 122, and a light chain variable domain comprising SEQ ID NOS: 206. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 124, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 128, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 128, and a light chain variable domain comprising SEQ ID NOS: 206. Further provided herein is an antibody or antigen binding fragment thereof that binds to tumor necrosis factor-like protein 1A (TL1A), comprising a heavy chain variable domain comprising SEQ ID NO: 129, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 130, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 131, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 132, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 133, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 134, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 135, and a light chain variable domain comprising SEQ ID NOS: 205. Further provided herein is an antibody or antigen binding fragment thereof that binds to tumor necrosis factor-like protein 1A (TL1A), comprising a heavy chain variable domain comprising SEQ ID NO: 132, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 126, and a light chain variable domain comprising SEQ ID NOS: 201. Further provided herein is an antibody or antigen binding fragment thereof that binds to tumor necrosis factor-like protein 1A (TL1A), comprising a heavy chain variable domain comprising SEQ ID NO: 130, and a light chain variable domain comprising SEQ ID NOS: 201.

Further provided herein is an antibody or antigen binding fragment thereof that binds to tumor necrosis factor-like protein 1A (TL1A), comprising a heavy chain domain comprising any one of SEQ ID NOs: 501-513, and a light chain domain comprising SEQ ID NOS: 514.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; (f) an LCDR3 comprising an amino acid sequence set forth by any one of SEQ ID NOS: 12-15; and a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1. In some embodiments, the human IgG1 comprises SEQ ID NO: 320. In some embodiments, the ADCC function of the Fc region comprising reduced ADCC is at least about 50% reduced as compared to human IgG1. In some embodiments, the CDC function of the Fc region comprising reduced ADCC is at least about 50% reduced as compared to human IgG1 In some embodiments, the Fc region comprises a human IgG1 comprising (a) 297A, 297Q, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some embodiments, the antibody of antigen binding fragment comprises a (i) human IgG4 Fc region or (ii) a human IgG4 Fc region comprising (a) S228P and L235E, or (b) S228P, F234A, and L235A, per Kabat numbering. In some embodiments, the antibody of antigen binding fragment comprises a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG26). In some embodiments, the antibody of antigen binding fragment comprises a human Fc region comprising high mannose glycosylation. In some embodiments, the Fe region comprises a human IgG1 with a substitution selected from 297A, 297Q, 297D, 279F, 279K, 279L, 228P, 235A, 235E, 235G, 235Q, 235R, 235S, 237A, 237E, 237K, 237N, 237R, 268K, 269N, 269Q, 270A, 270G, 270M, 270N, 424H, 424M, and 424V, per Kabat numbering. In some embodiments, the Fc region comprises a human IgG1 with a substitution selected from 271T, 272N, 292E, 292F, 292G, 292I, 293S, 301W, 304E, 311E, 311G, 311S, 255N, 256H, 256K, 256R, 256V, 316F, 328V, 330R, 339E, 339L, 343I, 343V, 373A, 373G, 373S, 376E, 376W, 376Y, 380D, 382D, 382P, 385P, 234A, 234V, 234F, 233P, 328A, 327Q and 327T, per Kabat numbering. In some embodiments, the Fc region comprises a human IgG1 with a substitution selected from 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering. In some embodiments, the antibody or antigen binding fragment comprises a sequence at least about 90%, 91%, 92%, 93%, 94%, 95%, 96%, 97%, 98%, 99%, or 100% identical to any one of SEQ ID NOS: 320-362. In some embodiments, HCDR1 comprises SEQ ID NO: 1. In some embodiments, HCDR2 comprises SEQ ID NO: 2. In some embodiments, HCDR2 comprises SEQ ID NO: 3. In some embodiments, HCDR2 comprises SEQ ID NO: 4. In some embodiments, HCDR2 comprises SEQ ID NO: 5. In some embodiments, HCDR3 comprises SEQ ID NO: 6. In some embodiments, HCDR3 comprises SEQ ID NO: 7. In some embodiments, HCDR3 comprises SEQ ID NO: 8. In some embodiments, HCDR3 comprises SEQ ID NO: 9. In some embodiments, LCDR1 comprises SEQ ID NO: 10. In some embodiments, LCDR2 comprises SEQ ID NO: 11. In some embodiments, LCDR3 comprises SEQ ID NO: 12. In some embodiments, LCDR3 comprises SEQ ID NO: 13. In some embodiments, LCDR3 comprises SEQ ID NO: 14. In some embodiments, LCDR3 comprises SEQ ID NO: 15.

In another aspect, provided herein is an antibody or antigen binding fragment thereof that binds to TL1A, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15. In some embodiments, the heavy chain variable region comprises human IGHV1-46*02 framework or a modified human IGHV1-46*02 framework. In some embodiments, the light chain variable framework region comprising a human IGKV3-20 framework or a modified human IGKV3-20 framework. In some embodiments, the heavy chain variable region comprises one of more of the following amino acids: 1E, 45K, 47R, 55I, 78A, 80I, 82T, 89A, 91L, per Aho or Kabat numbering. In some embodiments, the heavy chain variable region comprises 1E. In some embodiments, the heavy chain variable region comprises 45K. In some embodiments, the heavy chain variable region comprises 47R. In some embodiments, the heavy chain variable region comprises 55I. In some embodiments, the heavy chain variable region comprises 78A. In some embodiments, the heavy chain variable region comprises 80I. In some embodiments, the heavy chain variable region comprises 82T. In some embodiments, the heavy chain variable region comprises 89A. In some embodiments, the heavy chain variable region comprises 91L. In some embodiments, the light chain variable region comprises one or more of the following amino acids: 54P and 55W, per Aho or Kabat numbering. In some embodiments, the light chain variable region comprises 54P. In some embodiments, the light chain variable region comprises 55 W. In some embodiments, the HCDR2 comprises SEQ ID NO: 2. In some embodiments, the HCDR2 comprises SEQ ID NO: 3. In some embodiments, the HCDR2 comprises SEQ ID NO: 4. In some embodiments, the HCDR2 comprises SEQ ID NO: 5. In some embodiments, the HCDR3 comprises SEQ ID NO: 6. In some embodiments, the HCDR3 comprises SEQ ID NO: 7. In some embodiments, the HCDR3 comprises SEQ ID NO: 8. In some embodiments, the HCDR3 comprises SEQ ID NO: 9. In some embodiments, the LCDR3 comprises SEQ ID NO: 12. In some embodiments, the LCDR3 comprises SEQ ID NO: 13. In some embodiments, the LCDR3 comprises SEQ ID NO: 14. In some embodiments, LCDR3 comprises SEQ ID NO: 15.

In some embodiments, an antibody or antigen binding fragment described herein comprises a human IgG1 Fc region comprising (a) 297A, 297Q, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some embodiments, an antibody of antigen binding fragment herein comprises a (i) human IgG4 Fc region or (ii) a human IgG4 Fc region comprising (a) S228P and L235E, or (b) S228P, F234A, and L235A, per Kabat numbering. In some embodiments, an antibody of antigen binding fragment herein comprises a human IgG2 Fc region; IgG2-IgG4 cross-subclass Fc region; IgG2-IgG3 cross-subclass Fe region; IgG2 comprising H268Q, V309L, A330S, P331S (IgG2m4); or IgG2 comprising V234A, G237A, P238S, H268A, V309L, A330S, P331S (IgG2(a). In some embodiments, an antibody of antigen binding fragment herein comprises a human Fc region comprising high mannose glycosylation. In some embodiments, any of the antibody or antigen binding fragments described herein comprise a human IgG4 Fe region.

In some embodiments, an antibody or antigen binding fragment described herein comprises a human IgG1 with a substitution selected from 297A, 297Q, 297D, 279F, 279K, 279L, 228P, 235A, 235E, 235G, 235Q, 235R, 235S, 237A, 237E, 237K, 237N, 237R, 268K, 269N, 269Q, 270A, 270G, 270M, 270N, 424H, 424M, and 424V, per Kabat numbering. In some embodiments, an antibody or antigen binding fragment described herein comprises a human IgG1 with a substitution selected from 271T, 272N, 292E, 292F, 292G, 292I, 293S, 301W, 304E, 311E, 311G, 311S, 255N, 256H, 256K, 256R, 256V, 316F, 328V, 330R, 339E, 339L, 343I, 343V, 373A, 373G, 373S, 376E, 376W, 376Y, 380D, 382D, 382P, 385P, 234A, 234V, 234F, 233P, 328A, 327Q and 327T, per Kabat numbering. In some embodiments, an antibody or antigen binding fragment described herein comprises a human IgG1 with a substitution selected from 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering.

In some embodiments, an antibody or antigen binding fragment described herein comprises a Fc region comprising a sequence at least about 90%, 91%, 92%, 93%, 94%, 95%, 96%, 97%, 98%, 99%, or 100% identical to any one of SEQ ID NOS: 320-362.

In some embodiments, an antibody or antigen binding fragment described herein comprises at least about 80% monomeric fraction as determined by size exclusion chromatography. In some embodiments, an antibody or antigen binding fragment described herein comprises at least about 85%, 90%, 91%, 92%, 93%, 94%, 95%, 96%, 97%, 98%, or 99% monomeric fraction. In some embodiments, the size exclusion chromatography comprises injecting purified antibody or antigen binding fragment onto a size exclusion column, wherein the antibody or antigen binding fragment is purified by protein A. In some embodiments, the antibody or antigen binding fragment is purified as described in Example 2. In some embodiments, the antibody or antigen binding fragment is expressed under conditions described in Example 2. In some embodiments, the size exclusion chromatography column has an inner diameter of 4.6 mm. In some embodiments, the size exclusion chromatography column has a length of 150 mm. In some embodiments, the size exclusion chromatography column has a pore size of 200 Å. In some embodiments, the size exclusion chromatography column has a particle size of 1.7 micrometer. In some embodiments, the size exclusion chromatography column is ACQUITY UPLC BEH200 SEC column. In some embodiments, the antibody or antigen binding fragment is injected at a total volume of 15 μL. In some embodiments, the antibody or antigen binding fragment is injected at a concentration of about 0.1 μg/μL to about 1.0 μg/μL. In some embodiments, the size exclusion chromatography is performed on a Shimadzu UPLC instrument. In some embodiments, the size exclusion chromatography is performed at a flow rate of 0.2 mL/min. In some embodiments, the size exclusion chromatography is performed at a column oven temperature of 30° C. In some embodiments, the percentage of monomer is calculated using Shimadzu software. In some embodiments, the size exclusion chromatography is performed as described in Example 2.

In some embodiments, an antibody or antigen binding fragment described herein expresses at least about 20 μg/ml total antibody. In some embodiments, an antibody or antigen binding fragment described herein expresses between about 20 μg/ml and 70 μg/mL total antibody. In some embodiments, the antibody or antigen binding fragment is expressed in FreeStyle 293-F cells. In some embodiments, the antibody or antigen binding fragment is expressed as described in Example 2. In some embodiments, the antibody or antigen binding fragment expression level is quantified using Enzyme-Linked Immunosorbent assay (ELISA). In some embodiments, the ELISA comprises coating a surface of a substrate with a capture antibody that binds to a human or humanized antibody, applying the antibody or antigen binding fragment to the substrate, and applying to the substrate a second antibody that binds to a human or humanized antibody. In some embodiments, the capture antibody comprises an anti-kappa antibody. In some embodiments, the second antibody comprises an anti-Fc antibody. In some embodiments, the ELISA is performed as described in Example 2.

Further provided herein are methods of treating inflammatory bowel disease (IBD) in a subject in need thereof, the method comprising administering to the subject an antibody or antigen binding fragment described herein. In some embodiments, the IBD comprises Crohn's Disease. In some embodiments, the IBD comprises ulcerative colitis.

Also provided are antibodies or antigen binding fragments thereof that bind to tumor necrosis factor-like protein 1A (TL1A), comprising a heavy chain variable domain comprising an amino acid sequence at least 96% identical to SEQ ID NO: 104, and a light chain variable domain comprising an amino acid sequence at least 97% identical to SEQ ID NO: 201. In some embodiments, the heavy chain variable domain comprises an amino acid sequence at least 97% identical to SEQ ID NO: 104. In some embodiments, the heavy chain variable domain comprises an amino acid sequence at least 98% identical to SEQ ID NO: 104. In some embodiments, the heavy chain variable domain comprises an amino acid sequence at least 99% identical to SEQ ID NO: 104. In some embodiments, the heavy chain variable domain comprises SEQ ID NO: 104. In some embodiments, the light chain variable domain comprises an amino acid sequence at least 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Further provided are antibodies or antigen binding fragments thereof that bind to tumor necrosis factor-like protein 1A (TL1A), comprising a heavy chain variable domain comprising an amino acid sequence at least about 99% identical to any one of SEQ ID NOS: 101-135, and a light chain variable domain comprising an amino acid sequence at least about 99% identical to any one of SEQ ID NOS: 201-206. Table 16 and Table 17 set forth exemplary variable region amino acid sequences of anti-TL1A antibodies.

Provided are also antibodies or antigen binding fragments, as described herein, further comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ⊐, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. In some embodiments, the antibodies comprise a human IgG4 Fc region. In some embodiments, the antibodies a Fc region comprising a sequence at least about 90%, 91%, 92%, 93%, 94%, 95%, 96%, 97%, 98%, 99%, or 100% identical to any one of SEQ ID NOS: 320-362. In some embodiments, the antibodies comprise at least about 80% monomeric fraction as determined by size exclusion chromatography. In some embodiments, the antibodies are expressed in an amount at least about 20 μg/ml total antibody, optionally about 20 μg/ml and 70 μg/mL total antibody.

### 5.4.1.6 Antibody Properties

In various embodiments, the anti-TL1A antibody is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor. In certain embodiments, the antibodies inhibit TL1A activation as measured by interferon gamma release in human blood. In certain embodiments, the antibody inhibits interferon gamma release in human blood at an IC₅₀ of between about 1 nanomolar and about 30 picomolar. In certain embodiments, the antibody inhibits interferon gamma release in human blood at an IC₅₀ of between about 500 picomolar and about 30 picomolar. In certain embodiments, the antibody inhibits interferon gamma release in human blood at an IC₅₀ of between about 200 picomolar and about 30 picomolar. In certain embodiments, the antibody inhibits interferon gamma release in human blood at an IC₅₀ of less than or equal to about 200 picomolar. In certain embodiments, the antibody inhibits interferon gamma release in human blood at an IC₅₀ of less than or equal to about 100 picomolar.

In various embodiments, an anti-TL1A antibody provided herein has a binding affinity to human TL1A of less than about 1E⁻⁷, 1E⁻⁸, 1E⁻⁹, or 1E⁻¹⁰ Kd. In some cases, the binding affinity is from about 1E⁻⁹ to about 1E⁻¹⁰ Kd. In some embodiments, an anti-TL1A antibody provided herein has a binding affinity to murine TL1A and/or rat TL1A of less than about 1E⁻⁷, 1E⁻⁸, 1E⁻⁹, 1E⁻¹⁰, or 1E⁻¹¹ Kd. Methods for determining binding affinity are exemplified herein, including in Example 2.

In various embodiments, an anti-TL1A antibody provided herein comprises at least about 80% monomeric fraction after expression and purification as described in Example 2 or elsewhere herein. In various embodiments, an anti-TL1A antibody provided herein comprises at least about 85% monomeric fraction after expression and purification as described in Example 2 or elsewhere herein. In various embodiments, an anti-TL1A antibody provided herein comprises at least about 90% monomeric fraction after expression and purification as described in Example 2 or elsewhere herein. In various embodiments, an anti-TL1A antibody provided herein comprises at least about 91%, 92%, 93%, 94%, 95%, 96%, 97%, 98%, 99%, or 100% monomeric fraction after expression and purification as described in Example 2 or elsewhere herein.

In various embodiments, an anti-TL1A antibody provided herein has at least about 2 μg/mL expression as determined by the method disclosed herein. In some embodiments, the anti-TL1A antibody has about 2 μg/mL to about 60 μg/mL expression as determined by the method disclosed herein. In some embodiments, the anti-TL1A antibody has about 5 μg/mL to about 60 μg/mL expression as determined by the method disclosed herein. In some embodiments, the anti-TL1A antibody has about 10 μg/mL to about 60 μg/mL expression as determined by the method disclosed herein. In some embodiments, the anti-TL1A antibody has at least about 5 μg/mL expression as determined by the method disclosed herein. In some embodiments, the anti-TL1A antibody has at least about 10 μg/mL expression as determined by the method disclosed herein. In some embodiments, the anti-TL1A antibody has at least about 15 μg/mL expression as determined by the method disclosed herein. In some embodiments, the anti-TL1A antibody has at least about 20 μg/mL expression as determined by the method disclosed herein. In some embodiments, the anti-TL1A antibody expresses between about 2 μg/mL and about 50 μg/mL, between about 2 μg/mL and about 40 μg/mL, between about 2 μg/mL and about 30 μg/mL expression, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL as determined by the method disclosed herein. In some embodiments, the anti-TL1A antibody has about 2, 3, 4, 5, 6, 7, 8, 9, 10, 11, 12, 13, 14, 15, 16, 17, 18, 19, 20, 21, 22, 23, 24, 25, 26, 27, 28, 29, 30 μg/mL expression as determined by the method disclosed herein. Methods disclosed herein include those described in Example 2.

In various embodiments, an anti-TL1A antibody provided herein is humanized and has less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. The humanized heavy chain variable domain may comprise IGHV1-46*02 framework with no or fewer than about 10, 9, 8, 7, 6, 5, 4, 3, 2, or 1 non-human mutations. The humanized light chain variable domain may comprise IGKV3-20 framework with no or fewer than about 10, 9, 8, 7, 6, 5, 4, 3, 2, or 1 non-human mutations.

### 5.4.2 Assays

An exemplary screening paradigm for identification of antibody variants that express well in mammalian cells and preserve TL1A binding activity while minimizing the propensity of the antibody to aggregate comprises a five-step process. This screen was performed as detailed in the examples. Briefly, (1) variants were cloned and transiently expressed as intact Ig in 293 cells using small-scale (3 mL, 6-well culture plates) transfections, (2) the expression level of the antibody was assessed in the culture supernatant 96-120 hours after transfection using an antibody quantitation ELISA, (3) the binding of the supernatant antibody variants to human TL1A was assessed by ELISA, (4) the antibody was purified in a single step using Protein A and (5) the material was analyzed by analytical SEC to assess monomer/aggregate content. This approach enabled identification of variants that expressed well, preserved binding to TL1A, and displayed high monomer content.

Further provided herein are methods for analyzing antibody solubility based on percentage of monomeric fraction. For example, as described in Example 2.

Further provided herein are assays for quantifying antibody expression. For example, as described in Example 2.

Further provided herein are assays for quantifying immunogenicity of an antibody.

The antibodies described herein can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are provided in for e.g., Ausubel et al., eds, 1994, Current Protocols in Molecular Biology, Vol. 1, John Wiley & Sons, Inc., New York.

### 5.4.3 Additional Therapeutic Agent

A therapeutic agent may be used alone or in combination with an additional therapeutic agent. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

### 5.4.4 Methods of Generating Antibodies

In various embodiments, monoclonal antibodies are prepared using methods known in the art, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the anti-TL1A monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the anti-TL1A antibody is a human antibody. Human antibodies can be directly prepared using various techniques known in the art. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A. In addition, methods can be adapted for the construction of Fab expression libraries to allow rapid and effective identification of monoclonal Fab fragments with the desired specificity for TL1A, or derivatives, fragments, analogs or homologs thereof. Antibody fragments may be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO-S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 1303), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42: 133, 1986); Bauer et al. (Gene 37:73, 1985); Craik (BioTechniques, January 1985, 12-19); Smith et al. (Genetic Engineering: Principles and Methods, Plenum Press, 1981).

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

### 5.5 Pharmaceutical Compositions, Administration and Dosage

The anti-TL1A antibodies provided are useful in a variety of applications including, but not limited to, therapeutic treatment methods, such as the treatment of IBD. The methods of use may be in vitro, ex vivo, or in vivo methods. In certain embodiments, the anti-TL1A antibody is an antagonist for TL1A receptors.

In certain embodiments, the disease treated with anti-TL1A antibody or TL1A receptor antagonist is IBD, CD, UC and/or MR-UC.

In various embodiments, the pharmaceutical compositions are formulated for delivery via any route of administration. “Route of administration” may refer to any administration pathway known in the art, including but not limited to aerosol, nasal, oral, transmucosal, transdermal or parenteral.

The pharmaceutical compositions can also contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of an anti-TL1A antibody. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent (i.e. antibody or fragment thereof) used that will be effective in the treatment of a particular disorder or condition will depend on the nature of the disorder or condition and can be determined by one of skill in the art with standard clinical techniques.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

For the treatment of the disease, the appropriate dosage of an antibody depends on the type of disease to be treated, the severity and course of the disease, the responsiveness of the disease, whether the antibody is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The TL1A antibody can be administered one time or over a series of treatments lasting from several days to several months, or until a cure is effected or a diminution of the disease state is achieved (e.g., treatment or amelioration of IBD). The duration of treatment depends upon the subject's clinical progress and responsiveness to therapy. In certain embodiments, dosage is from 0.01 μg to 100 mg per kg of body weight, and can be given once or more daily, weekly, monthly or yearly.

Antibody therapeutics suitable for injection and/or administration are important to realizing the full therapeutic potential of mAbs (monoclonal antibodies, e.g. an anti-TL1A monoclonal antibody). However, administration is generally restricted by volume. This, in turn, elucidates the importance developing of high concentration mAb formulations of greater than, for example in some cases, 100 mg/ml. Problems associated with mAb development include high solution viscosity and opalescence, which are commonly encountered during the development of high-concentration (e.g. greater than 100 mg/ml). Both viscosity and opalescence impact mAb developability broadly, affecting manufacturability, stability, and delivery. High solution viscosities (e.g. greater than 30 mPa-s) cause limiting back-pressures in ultrafiltration/diafiltration during the mAb concentration unit operation. Similarly, viscous mAb solutions also result in forbidding or incompatible injection forces when administering via injection, including via patient friendly autoinjectors. In effect, solution viscosity can be a determining factor for the maximum mAb dose possible via injection. Solution opalescence in therapeutic mAbs can be equally problematic as opalescence can indicate predisposition for liquid-liquid phase separation, precipitation, or aggregation

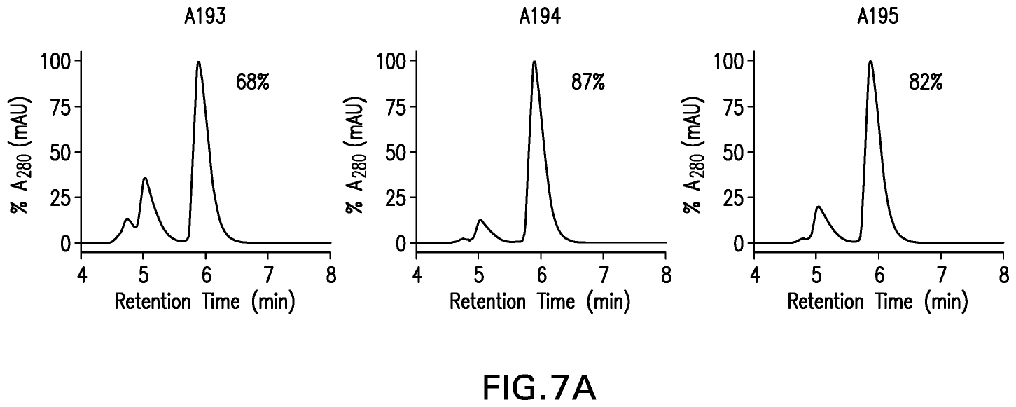

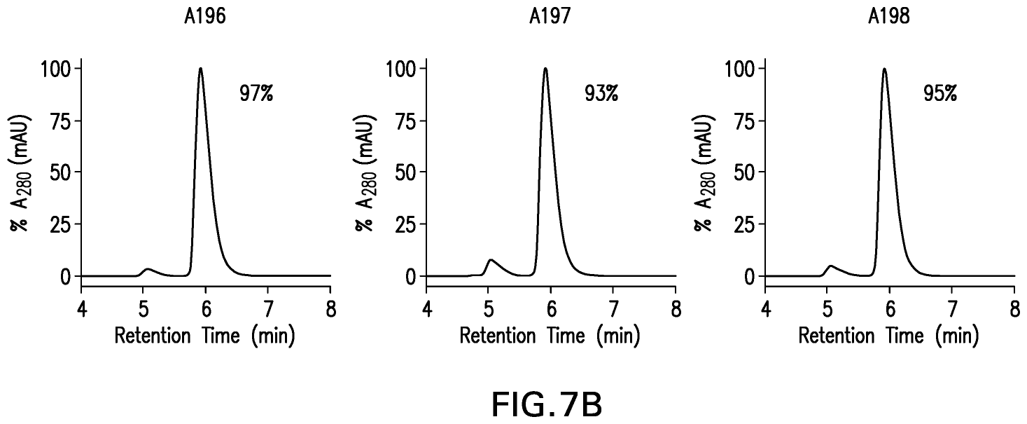

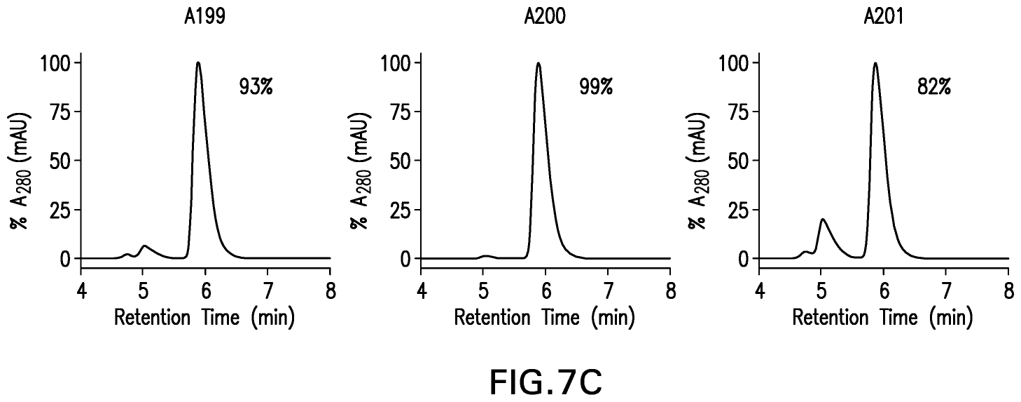

The anti-TL1A antibodies provided herein demonstrate advantageous viscosity and aggregation properties at high antibody concentrations (e.g. greater than 100 mg/mL or greater than 150 mg/mL). Notably, anti-TL1A antibodies provided herein are characterized by low viscosity (e.g. less than 10 mPa-s) and low aggregation (e.g. less than 5% high molecular weight species) at high concentrations (FIGS. 7A-7C).

For example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-T1LA antibody is characterized by a viscosity less than about 30, 20, 15, or 10 mPa-s at a concentration greater than about 100 mg/mL, e.g., up to about 170 mg/mL. In some embodiments, the anti-T1LA antibody is characterized by a viscosity less than about 30, 20, 15, or 10 mPa-s at a concentration greater than at least about 100 mg/mL. In some embodiments, the anti-T1LA antibody is characterized by a viscosity less than about 30, 20, 15, or 10 mPa-s at a concentration up to about 170 mg/mL. In some embodiments, the anti-T1LA antibody is characterized by a viscosity less than about 30, 20, 15, or 10 mPa-s at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-T1LA antibodies is characterized by a viscosity less than about 30, 20, 15, or 10 mPa-s at a concentration about or greater than about 100 mg/mL, about 125 mg/mL, about 150 mg/mL, about 160 mg/mL, or about 170 mg/mL. In some embodiments, less than about 10 mPa-s includes from about 4 to about 10 mPa-s, from about 4 to about 9 mPa-s, from about 4 to about 8 mPa-s, from about 4 to about 7 mPa-s, from about 4 to about 6 mPa-s, from about 4 to about 5 mPa-s, from about 5 to about 10 mPa-s, from about 5 to about 9 mPa-s, from about 5 to about 8 mPa-s, from about 5 to about 7 mPa-s, from about 5 to about 6 mPa-s, from about 6 to about 10 mPa-s, from about 6 to about 9 mPa-s, from about 6 to about 8 mPa-s, or from about 6 to about 7 mPa-s. In some embodiments, greater than about 100, 125, 150, or 160 mg/ml is up to about 170 mg/ml.

Additionally, for example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody is characterized by a turbidity less than 12 Nephelometric Turbidity Units (NTU) when at a concentration greater than about 100 mg/mL to about 170 mg/mL. In some embodiments, the anti-TL1A antibody is characterized by a turbidity less than 12 Nephelometric Turbidity Units (NTU) when at a concentration greater than at least about 100 mg/mL. In some embodiments, the anti-TL1A antibody is characterized by a turbidity less than 12 Nephelometric Turbidity Units (NTU) when at a concentration up to about 170 mg/mL. In some embodiments, the anti-TL1A antibody is characterized by a turbidity less than 12 Nephelometric Turbidity Units (NTU) when at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody is characterized by a turbidity less than 12 Nephelometric Turbidity Units (NTU) when at a concentration at or greater than about 100 mg/mL, about 125 mg/mL, about 150 mg/mL, about 160 mg/mL, or about 170 mg/mL.

By way of further example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-T1LA antibody at a concentration from about 150 mg/mL to about or greater than about 170 mg/mL is characterized by a viscosity less than about 10 mPa-s to about 30 mPa-s. In some embodiments, the anti-T1LA antibody at a concentration from about 150 mg/mL to about or greater than about 170 mg/mL is characterized by a viscosity less than about 30 mPa-s. In some embodiments, the anti-T1LA antibody at a concentration from about 150 mg/mL to about or greater than about 170 mg/mL is characterized by a viscosity less than about 5 mPa-s to about 10 mPa-s, about 5 mPa-s to about 15 mPa-s, about 5 mPa-s to about 20 mPa-s, about 5 mPa-s to about 30 mPa-s, about 10 mPa-s to about 15 mPa-s, about 10 mPa-s to about 20 mPa-s, about 10 mPa-s to about 30 mPa-s, about 15 mPa-s to about 20 mPa-s, about 15 mPa-s to about 30 mPa-s, about 20 mPa-s to about 30 mPa-s, about 5 mPa-s to about 9 mPa-s, about 4 to about 10 mPa-s, about 4 to about 9 mPa-s, about 4 to about 8 mPa-s, about 4 to about 7 mPa-s, about 4 to about 6 mPa-s, about 4 to about 5 mPa-s, about 5 to about 10 mPa-s, about 5 to about 9 mPa-s, about 5 to about 8 mPa-s, about 5 to about 7 mPa-s, about 5 to about 6 mPa-s, about 6 to about 10 mPa-s, about 6 to about 9 mPa-s, about 6 to about 8 mPa-s, or about 6 to about 7 mPa-s. In some embodiments, the anti-T1LA antibody at a concentration of about 150 mg/mL to about or greater than about 170 mg/mL is characterized by a viscosity less than about 5 mPa-s, about 10 mPa-s, about 15 mPa-s, about 20 mPa-s, or about 30 mPa-s. In some embodiments, less than about 5, 10, 15, 20, or 30 mPa-s is at least about 1 mPa-s.

Additionally, for example, for an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody having a concentration greater than 150 mg/mL is characterized by a turbidity less than about 5 Nephelometric Turbidity Units (NTU) to about 15 Nephelometric Turbidity Units (NTU). In some embodiments, the anti-TL1A antibody having a concentration greater than 150 mg/mL is characterized by a turbidity less than at least about 5 Nephelometric Turbidity Units (NTU). In some embodiments, the anti-TL1A antibody having a concentration greater than 150 mg/mL is characterized by a turbidity less than at most about 15 Nephelometric Turbidity Units (NTU). In some embodiments, the anti-TL1A antibody having a concentration greater than 150 mg/mL is characterized by a turbidity less than about 5 Nephelometric Turbidity Units (NTU) to about 7.5 Nephelometric Turbidity Units (NTU), about 5 Nephelometric Turbidity Units (NTU) to about 10 Nephelometric Turbidity Units (NTU), about 5 Nephelometric Turbidity Units (NTU) to about 12.5 Nephelometric Turbidity Units (NTU), about 5 Nephelometric Turbidity Units (NTU) to about 15 Nephelometric Turbidity Units (NTU), about 7.5 Nephelometric Turbidity Units (NTU) to about 10 Nephelometric Turbidity Units (NTU), about 7.5 Nephelometric Turbidity Units (NTU) to about 12.5 Nephelometric Turbidity Units (NTU), about 7.5 Nephelometric Turbidity Units (NTU) to about 15 Nephelometric Turbidity Units (NTU), about 10 Nephelometric Turbidity Units (NTU) to about 12.5 Nephelometric Turbidity Units (NTU), about 10 Nephelometric Turbidity Units (NTU) to about 15 Nephelometric Turbidity Units (NTU), or about 12.5 Nephelometric Turbidity Units (NTU) to about 15 Nephelometric Turbidity Units (NTU). In some embodiments, the anti-TL1A antibody having a concentration greater than 150 mg/mL is characterized by a turbidity less than about 5 Nephelometric Turbidity Units (NTU), about 7.5 Nephelometric Turbidity Units (NTU), about 10 Nephelometric Turbidity Units (NTU), about 12.5 Nephelometric Turbidity Units (NTU), or about 15 Nephelometric Turbidity Units (NTU).

The anti-TL1A antibodies described herein also demonstrate advantageous aggregation properties. For an antibody or antigen binding fragment wherein HCDR1 comprises SEQ ID NO: 1, HCDR2 comprises SEQ ID NO: 2, HCDR3 comprises SEQ ID NO: 6, LCDR1 comprises SEQ ID NO: 10, LCDR2 comprises SEQ ID NO: 11, and LCDR3 comprises SEQ ID NO: 12 or an antibody or antigen binding fragment wherein the heavy chain variable region comprises SEQ ID NO: 104 and the light chain variable region comprises SEQ ID NO: 201, in some embodiments, the anti-TL1A antibody composition is characterized by percent high molecular weight species (e.g. a species having a molecular weight greater than the molecular weight of the monomer)) less than 10% when at a concentration greater than about 100 mg/mL to about greater than 170 mg/mL. In some embodiments, the anti-TL1A antibody composition is characterized by percent high molecular weight species less than 10% when at a concentration greater than at least about 100 mg/mL. In some embodiments, the anti-TL1A antibody composition is characterized by percent high molecular weight species less than 10% when at a concentration up to about 170 mg/mL. In some embodiments, the anti-TL1A antibody composition is characterized by percent high molecular weight species less than 10% when at a concentration from about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody composition is characterized by percent high molecular weight species less than 10% when at a concentration about or greater than about 100 mg/mL, about 125 mg/mL, about 150 mg/mL, about 160 mg/mL, or about 170 mg/mL. In some embodiments, the anti-TL1A antibody composition having an antibody concentration greater than 150 mg/mL is characterized by a high molecular weight species less than about 5% to about 15%. In some embodiments, the anti-TL1A antibody composition having an antibody concentration greater than 150 mg/mL is characterized by a high molecular weight species less than at most about 15%. In some embodiments, the anti-TL1A antibody composition having an antibody concentration greater than 150 mg/mL is characterized by a high molecular weight species less than about 5% to about 7.5%, about 5% to about 10%, about 5% to about 15%, about 5% to about 17.5%, about 5% to about 20%, about 5% to about 25%, about 7.5% to about 10%, about 7.5% to about 15%, about 7.5% to about 17.5%, about 7.5% to about 20%, about 7.5% to about 25%, about 10% to about 15%, about 10% to about 17.5%, about 10% to about 20%, about 10% to about 25%, about 15% to about 17.5%, about 15% to about 20%, about 15% to about 25%, about 17.5% to about 20%, about 17.5% to about 25%, or about 20% to about 25%. In some embodiments, the anti-TL1A antibody composition having an antibody concentration greater than 150 mg/mL is characterized by a high molecular weight species less than about 5%, about 7.5%, about 10%, about 15%, about 17.5%, about 20%, or about 25%.

By way of further example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-T1LA antibodies is characterized by a viscosity less than 10 mPa-s at a concentration greater than about 100 mg/mL to about greater than 170 mg/mL. In some embodiments, the anti-T1LA antibodies is characterized by a viscosity less than 10 mPa-s at a concentration greater than at least about 100 mg/mL. In some embodiments, the anti-TILA antibodies is characterized by a viscosity less than 10 mPa-s at a concentration greater than at most about 170 mg/mL. In some embodiments, the anti-TILA antibodies is characterized by a viscosity less than 10 mPa-s at a concentration greater than about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-T1LA antibodies is characterized by a viscosity less than 10 mPa-s at a concentration greater than about 100 mg/mL, about 125 mg/mL, about 150 mg/mL, about 160 mg/mL, or about 170 mg/mL.

Additionally, for example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody is characterized by a turbidity less than 12 Nephelometric Turbidity Units (NTU) when at a concentration greater than about 100 mg/mL to about greater than 170 mg/mL. In some embodiments, the anti-TL1A antibody is characterized by a turbidity less than 12 Nephelometric Turbidity Units (NTU) when at a concentration greater than at least about 100 mg/mL. In some embodiments, the anti-TL1A antibody is characterized by a turbidity less than 12 Nephelometric Turbidity Units (NTU) when at a concentration greater than at most about 170 mg/mL. In some embodiments, the anti-TL1A antibody is characterized by a turbidity less than 12 Nephelometric Turbidity Units (NTU) when at a concentration greater than about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody is characterized by a turbidity less than 12 Nephelometric Turbidity Units (NTU) when at a concentration greater than about 100 mg/mL, about 125 mg/mL, about 150 mg/mL, about 160 mg/mL, or about 170 mg/mL.

Additionally, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-T1LA antibody at a concentration greater than 150 mg/mL to greater than about 170 mg/mL is characterized by a viscosity less than about 10 mPa-s to about 30 mPa-s. In some embodiments, the anti-T1LA antibody at a concentration greater than 150 mg/mL to greater than about 170 mg/mL is characterized by a viscosity less than at most about 30 mPa-s. In some embodiments, the anti-T1LA antibody at a concentration greater than 150 mg/mL to greater than about 170 mg/mL is characterized by a viscosity less than about 5 mPa-s to about 10 mPa-s, about 5 mPa-s to about 15 mPa-s, about 5 mPa-s to about 20 mPa-s, about 5 mPa-s to about 30 mPa-s, about 10 mPa-s to about 15 mPa-s, about 10 mPa-s to about 20 mPa-s, about 10 mPa-s to about 30 mPa-s, about 15 mPa-s to about 20 mPa-s, about 15 mPa-s to about 30 mPa-s, or about 20 mPa-s to about 30 mPa-s. In some embodiments, the anti-T1LA antibody at a concentration greater than 150 mg/mL to greater than about 170 mg/mL is characterized by a viscosity less than about 5 mPa-s, about 10 mPa-s, about 15 mPa-s, about 20 mPa-s, or about 30 mPa-s.

Additionally, for example, for an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody having a concentration greater than 150 mg/mL is characterized by a turbidity less than about 5 Nephelometric Turbidity Units (NTU) to about 15 Nephelometric Turbidity Units (NTU). In some embodiments, the anti-TL1A antibody having a concentration greater than 150 mg/mL is characterized by a turbidity less than at least about 5 Nephelometric Turbidity Units (NTU). In some embodiments, the anti-TL1A antibody having a concentration greater than 150 mg/mL is characterized by a turbidity less than at most about 15 Nephelometric Turbidity Units (NTU). In some embodiments, the anti-TL1A antibody having a concentration greater than 150 mg/mL is characterized by a turbidity less than about 5 Nephelometric Turbidity Units (NTU) to about 7.5 Nephelometric Turbidity Units (NTU), about 5 Nephelometric Turbidity Units (NTU) to about 10 Nephelometric Turbidity Units (NTU), about 5 Nephelometric Turbidity Units (NTU) to about 12.5 Nephelometric Turbidity Units (NTU), about 5 Nephelometric Turbidity Units (NTU) to about 15 Nephelometric Turbidity Units (NTU), about 7.5 Nephelometric Turbidity Units (NTU) to about 10 Nephelometric Turbidity Units (NTU), about 7.5 Nephelometric Turbidity Units (NTU) to about 12.5 Nephelometric Turbidity Units (NTU), about 7.5 Nephelometric Turbidity Units (NTU) to about 15 Nephelometric Turbidity Units (NTU), about 10 Nephelometric Turbidity Units (NTU) to about 12.5 Nephelometric Turbidity Units (NTU), about 10 Nephelometric Turbidity Units (NTU) to about 15 Nephelometric Turbidity Units (NTU), or about 12.5 Nephelometric Turbidity Units (NTU) to about 15 Nephelometric Turbidity Units (NTU). In some embodiments, the anti-TL1A antibody having a concentration greater than 150 mg/mL is characterized by a turbidity less than about 5 Nephelometric Turbidity Units (NTU), about 7.5 Nephelometric Turbidity Units (NTU), about 10 Nephelometric Turbidity Units (NTU), about 12.5 Nephelometric Turbidity Units (NTU), or about 15 Nephelometric Turbidity Units (NTU).

The anti-TL1A antibodies described herein also demonstrate advantageous aggregation properties. For an antibody or antigen binding fragment comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework, in some embodiments, the anti-TL1A antibody composition is characterized by percent high molecular weight species (e.g. a species having a molecular weight greater than the molecular weight of the monomer)) less than 10% when at a concentration greater than about 100 mg/mL to about greater than 170 mg/mL. In some embodiments, the anti-TL1A antibody composition is characterized by percent high molecular weight species less than 10% when at a concentration greater than at least about 100 mg/mL. In some embodiments, the anti-TL1A antibody composition is characterized by percent high molecular weight species less than 10% when at a concentration greater than at most about 170 mg/mL. In some embodiments, the anti-TL1A antibody composition is characterized by percent high molecular weight species less than 10% when at a concentration greater than about 100 mg/mL to about 125 mg/mL, about 100 mg/mL to about 150 mg/mL, about 100 mg/mL to about 160 mg/mL, about 100 mg/mL to about 170 mg/mL, about 125 mg/mL to about 150 mg/mL, about 125 mg/mL to about 160 mg/mL, about 125 mg/mL to about 170 mg/mL, about 150 mg/mL to about 160 mg/mL, about 150 mg/mL to about 170 mg/mL, or about 160 mg/mL to about 170 mg/mL. In some embodiments, the anti-TL1A antibody composition is characterized by percent high molecular weight species less than 10% when at a concentration greater than about 100 mg/mL, about 125 mg/mL, about 150 mg/mL, about 160 mg/mL, or about 170 mg/mL. In some embodiments, the anti-TL1A antibody composition having an antibody concentration greater than 150 mg/mL is characterized by a high molecular weight species less than about 5% to about 15%. In some embodiments, the anti-TL1A antibody composition having an antibody concentration greater than 150 mg/mL is characterized by a high molecular weight species less than at most about 15%. In some embodiments, the anti-TL1A antibody composition having an antibody concentration greater than 150 mg/mL is characterized by a high molecular weight species less than about 5% to about 7.5%, about 5% to about 10%, about 5% to about 15%, about 5% to about 17.5%, about 5% to about 20%, about 5% to about 25%, about 7.5% to about 10%, about 7.5% to about 15%, about 7.5% to about 17.5%, about 7.5% to about 20%, about 7.5% to about 25%, about 10% to about 15%, about 10% to about 17.5%, about 10% to about 20%, about 10% to about 25%, about 15% to about 17.5%, about 15% to about 20%, about 15% to about 25%, about 17.5% to about 20%, about 17.5% to about 25%, or about 20% to about 25%. In some embodiments, the anti-TL1A antibody composition having an antibody concentration greater than 150 mg/mL is characterized by a high molecular weight species less than about 5%, about 7.5%, about 10%, about 15%, about 17.5%, about 20%, or about 25%.

### 5.5.1 Dosages and Routes of Administration

In general, methods disclosed herein comprise administering a therapeutic agent by oral administration. However, in some instances, methods comprise administering a therapeutic agent by intraperitoneal injection. In some instances, methods comprise administering a therapeutic agent in the form of an anal suppository. In some instances, methods comprise administering a therapeutic agent by intravenous (“i.v.”) administration. It is conceivable that one may also administer therapeutic agents disclosed herein by other routes, such as subcutaneous injection, intramuscular injection, intradermal injection, transdermal injection percutaneous administration, intranasal administration, intralymphatic injection, rectal administration intragastric administration, or any other suitable renteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., step-wise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 pmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

### 5.6 Numbered Embodiments

In certain embodiments, described herein are methods for evaluating an effect of a treatment described herein. In some instances, the treatment comprises administration with an inhibitor of TL1A activity or expression and optionally, one or more additional therapeutic agents. In some instances, the treatment is monitored by evaluating the quantity of TL1A in the subject prior to and/or after administration of a therapeutic agent.
1. A method of inhibiting or reducing TL1A activity or expression in a subject having or suspected of having at least one of an inflammatory, fibrostenotic, and fibrotic, disease or condition the method comprising:
   a) identifying the subject as being a carrier of a genotype comprising a polymorphism provided in Table 1 or Table 4, or a polymorphism in linkage disequilibrium (LD) therewith; and
   b) administering to the subject a therapeutically effective amount of an anti-TL1A antibody from Tables 16-17 or 20, thereby inhibiting or reducing TL1A activity or expression in the subject.
2. The method of embodiment 1, provided that the inflammatory disease comprises Crohn's disease.
3. The method of embodiment 2, provided that the Crohn's disease comprises ileal, ileocolonic, or colonic Crohn's disease.
4. The method of embodiment 1, provided that the inflammatory disease comprises ulcerative colitis (UC).
5. The method of embodiment 4, provided that the UC is medically refractory UC.
6. The method of any of embodiments 1-5, wherein identifying the subject as being a carrier of the genotype of step (a) comprises:
   c) contacting a sample comprising genetic material from the subject with a nucleic acid sequence capable of hybridizing to at least 10 contiguous nucleobases comprising a risk allele located at nucleoposition 501 within any one of SEQ ID NOS: 2001-2048, or 2057-2059; and
   d) detecting binding between the nucleic acid sequence and the at least 10 contiguous nucleobases comprising the risk allele.
7. The method of embodiment 6, provided that the standard hybridization conditions comprise an annealing temperature between about 35° C. and about 65° C.
8. The method of embodiment 6 or embodiment 7, provided that the standard hybridization conditions are performed with a TaqMan master mix solution.
9. The method of any of embodiments 6-8, provided that the nucleic acid sequence is conjugated to a detectable molecule.
10. The method of embodiment 9, provided that the detectable molecule comprises a fluorophore.
11. The method of any of embodiments 6-10, provided that the nucleic acid sequence is conjugated to a quencher.
12. The method of any of embodiments 6-11, provided that the sample comprising genetic material from the subject is amplified genetic material obtained from a nucleic acid amplification assay.
13. The method of embodiment 12, provided that the nucleic acid amplification assay comprises amplification of DNA from the subject with a pair of primers capable of amplifying at least 15 contiguous nucleobases comprising the risk allele located at nucleoposition 501 within any one of SEQ ID NOS: 2001-2048, or 2057-2059, the pair of primers comprising a first primer and a second primer.
14. The method of embodiment 12, provided that the first primer comprises a nucleic acid sequence complimentary to at least 15 contiguous nucleobases upstream of the risk allele located at nucleobase 501 within any one of SEQ ID NOS: 2001-2048, or 2057-2059, and the second primer comprises a nucleic acid sequence complimentary to at least 15 contiguous nucleobases downstream of the risk allele located at nucleobase 501 within any one of SEQ ID NOS: 2001-2048, or 2057-2059.
15. The method of any of embodiments 1-14, provided that the subject has been determined to be a carrier of the genotype by a process comprising DNA sequencing.
16. The method of any of embodiments 1-15, provided that the subject further comprises soluble TL1A at a level greater than a control level derived from a non-diseased individual or population of non-diseased individuals.
17. The method of any of embodiments 1-16, provided that the subject is homozygous for the genotype.
18. The method of any of embodiments 1-17, wherein the genotype comprises at least two polymorphisms provided in Table 1 or Table 4.
19. The method of any of embodiments 1-17, wherein the genotype comprises at least three polymorphisms provided in Table 1 or Table 4.
20. The method of any of embodiments 1-17, wherein the genotype comprises at least four polymorphisms provided in Table 1 or Table 4.
21. The method of any of embodiments 1-17, wherein the genotype comprises at least five polymorphisms provided in Table 1 or Table 4.
22. The method of any of embodiments 1-17, wherein the genotype comprises at least six polymorphisms provided in Table 1 or Table 4.
23. The method of any of embodiments 1-17, wherein the genotype comprises at least seven polymorphisms provided in Table 1 or Table 4.
24. The method of any of embodiments 1-17, wherein the genotype comprises at least eight polymorphisms provided in Table 1 or Table 4.
25. The method of any of embodiments 1-24, wherein the genotype comprises a polymorphism selected from the group consisting of a “G” allele at rs11897732 (SEQ ID NO: 2001), an “A” allele at rs6740739 (SEQ ID NO: 2002), a “G” allele at rs17796285 (SEQ ID NO: 2003), an “A” allele at rs7935393 (SEQ ID NO: 2004), a “G” allele at rs12934476 (SEQ ID NO: 2005), an “A” allele at rs12457255 (SEQ ID NO: 2006), an “A” allele at rs2070557 (SEQ ID NO: 2007), an “A” allele at rs4246905 (SEQ ID NO: 2008), an “A” allele at rs10974900 (SEQ ID NO: 2009), a “C” allele at rs12434976 (SEQ ID NO: 2010), an “T” allele at rs16901748 (SEQ ID NO: 2011), an “A” allele at rs2815844 (SEQ ID NO: 2012), a “G” allele at rs889702 (SEQ ID NO: 2013), a “C” allele at rs2409750 (SEQ ID NO: 2014), an “A” allele at rs1541020 (SEQ ID NO: 2015), a “T” allele at rs4942248 (SEQ ID NO: 2016), a “G” allele at rs12934476 (SEQ ID NO: 2017), an “A” allele at rs12457255 (SEQ ID NO: 2018), an “A” allele at rs2297437 (SEQ ID NO: 2019), a “G” allele at rs41309367 (SEQ ID NO: 2020), an “A” allele at rs10733509 (SEQ ID NO: 2021), a “G” allele at rs10750376 (SEQ ID NO: 2022), a “G” allele at rs10932456 (SEQ ID NO: 2023), an “A” allele at rs1326860 (SEQ ID NO: 2024), a “G” allele at rs1528663 (SEQ ID NO: 2025), a “C” allele at rs1892231 (SEQ ID NO: 2026), an “A” allele at rs951279 (SEQ ID NO: 2027), an “A” allele at rs9806914 (SEQ ID NO: 2028), an “A” allele at rs7935393 (SEQ ID NO: 2029), a “G” allele at rs1690492 (SEQ ID NO: 2030), an “A” allele at rs420726 (SEQ ID NO: 2031), a “T” allele at rs7759385 (SEQ ID NO: 2032), an “A” allele at rs10974900 (SEQ ID NO: 2033), an “A” allele at rs1326860 (SEQ ID NO: 2034), a “C” allele at rs2548147 (SEQ ID NO: 2035), an “A” allele at rs2815844 (SEQ ID NO: 2036), a “G” allele at rs889702 (SEQ ID NO: 2037), an “A” allele at rs9806914 (SEQ ID NO: 2038), an “A” allele at rs6478109 (SEQ ID NO: 2039), a “C” allele at rs7278257 (SEQ ID NO: 2040), an “A” allele at rs11221332 (SEQ ID NO: 2041), an “A” allele at rs56124762 (SEQ ID NO: 2057), a “G” at rs2070558 (SEQ ID NO: 2058), and a “T” allele at rs2070561 (SEQ ID NO: 2059).
26. The method of embodiments 1-25, wherein the inhibitor of TL1A activity or expression is an anti-TL1A antibody.
27. The method of embodiment 26, wherein the anti-TL1A antibody is selected from Table 20.
28. The method of embodiment 26, wherein the anti-TL1A antibody comprises an amino acid sequence provided in Tables 16-17.
29. The method of embodiment 26, wherein the anti-TL1A antibody binds to the same region of human TL1A as a reference antibody selected from Table 20.
30. The method of embodiment 26, wherein the anti-TL1A antibody binds to the same region of human TL1A as a reference antibody, the reference antibody comprising an amino acid sequence provided in Tables 16-17.
31. The method of embodiments 26-30, wherein the anti-TL1A antibody is a neutralizing TL1A antibody.
32. The method of embodiments 26-31, wherein the anti-TL1A antibody is an antagonist of TL1A.
33. A method of treating an inflammatory, fibrostenotic, and fibrotic, disease or condition in a subject comprising administering to the subject a therapeutically effective amount of an inhibitor of TL1A activity or expression, provided a presence of a genotype is detected in a sample obtained from the subject.
34. A method of treating an inflammatory, fibrostenotic, and fibrotic, disease or condition in a subject comprising:
   e) analyzing a sample obtained from a subject to detect a presence or an absence of a genotype;
   f) detect the presence of the genotype in the sample obtained from the subject;
   g) administering to the subject a therapeutically effective amount of a TL1A antibody or antigen binding fragment from Tables 16-17 or 21.
35. The method of embodiment 33-34, provided that the inflammatory disease comprises Crohn's disease.
36. The method of embodiment 35, provided that the Crohn's disease comprises ileal, ileocolonic, or colonic Crohn's disease.
37. The method of embodiment 33-34, provided that the inflammatory disease is ulcerative colitis (UC).
38. The method of embodiment 37, provided that the UC is medically refractory UC.
39. The method of any of embodiments 33-38, wherein the presence of the genotype is detected in the sample obtained from the subject by:
   h) contacting the sample comprising genetic material from the subject with a nucleic acid sequence capable of hybridizing to at least 10 contiguous nucleobases comprising a risk allele located at nucleoposition 501 within any one of SEQ ID NOS: 2001-2048, or 2057-2059; and
   i) detecting binding between the nucleic acid sequence and the at least 10 contiguous nucleobases comprising the risk allele.
40. The method of embodiment 39, provided that the standard hybridization conditions comprise an annealing temperature between about 35° C. and about 65° C.
41. The method of embodiment 39 or embodiment 40, provided that the standard hybridization conditions are performed with a TaqMan master mix solution.
42. The method of any of embodiments 39-37, provided that the nucleic acid sequence is conjugated to a detectable molecule.
43. The method of embodiment 42, provided that the detectable molecule comprises a fluorophore.
44. The method of any of embodiments 39-43, provided that the nucleic acid sequence is conjugated to a quencher.
45. The method of any of embodiments 39-44, provided that the sample comprising genetic material from the subject is amplified genetic material obtained from a nucleic acid amplification assay.
46. The method of embodiment 45, provided that the nucleic acid amplification assay comprises amplification of DNA from the subject with a pair of primers capable of amplifying at least 15 contiguous nucleobases comprising the risk allele located at nucleoposition 501 within any one of SEQ ID NOS: 2001-2048, or 2057-2059, the pair of primers comprising a first primer and a second primer.
47. The method of embodiment 46, provided that the first primer comprises a nucleic acid sequence complimentary to at least 15 contiguous nucleobases upstream of the risk allele located at nucleobase 501 within any one of SEQ ID NOS: 2001-2048, or 2057-2059, and the second primer comprises a nucleic acid sequence complimentary to at least 15 contiguous nucleobases downstream of the risk allele located at nucleobase 501 within any one of SEQ ID NOS: 2001-2048, or 2057-2059.
48. The method of any of embodiments 34-47, the presence of the genotype is detected in the sample obtained from the subject by a process comprising DNA sequencing.
49. The method of any of embodiments 34-48, provided that the subject further comprises soluble TL1A at a level greater than a control level derived from a non-diseased individual or population of non-diseased individuals.
50. The method of any of embodiments 34-49, provided that the subject is homozygous for the genotype.
51. The method of any of embodiments 34-50, wherein the genotype comprises at least two polymorphisms provided in Table 1 or Table 4.
52. The method of any of embodiments 34-51, wherein the genotype comprises at least three polymorphisms provided in Table 1 or Table 4.
53. The method of any of embodiments 34-52, wherein the genotype comprises at least four polymorphisms provided in Table 1 or Table 4.
54. The method of any of embodiments 34-53, wherein the genotype comprises at least five polymorphisms provided in Table 1 or Table 4.
55. The method of any of embodiments 34-54, wherein the genotype comprises at least six polymorphisms provided in Table 1 or Table 4.
56. The method of any of embodiments 34-55, wherein the genotype comprises at least seven polymorphisms provided in Table 1 or Table 4.
57. The method of any of embodiments 34-56, wherein the genotype comprises at least eight polymorphisms provided in Table 1 or Table 4.
58. The method of any of embodiments 34-57, wherein the genotype comprises a polymorphism selected from the group consisting of a “G” allele at rs11897732 (SEQ ID NO: 2001), an “A” allele at rs6740739 (SEQ ID NO: 2002), a “G” allele at rs17796285 (SEQ ID NO: 2003), an “A” allele at rs7935393 (SEQ ID NO: 2004), a “G” allele at rs12934476 (SEQ ID NO: 2005), an “A” allele at rs12457255 (SEQ ID NO: 2006), an “A” allele at rs2070557 (SEQ ID NO: 2007), an “A” allele at rs4246905 (SEQ ID NO: 2008), an “A” allele at rs10974900 (SEQ ID NO: 2009), a “C” allele at rs12434976 (SEQ ID NO: 2010), an “T” allele at rs16901748 (SEQ ID NO: 2011), an “A” allele at rs2815844 (SEQ ID NO: 2012), a “G” allele at rs889702 (SEQ ID NO: 2013), a “C” allele at rs2409750 (SEQ ID NO: 2014), an “A” allele at rs1541020 (SEQ ID NO: 2015), a “T” allele at rs4942248 (SEQ ID NO: 2016), a “G” allele at rs12934476 (SEQ ID NO: 2017), an “A” allele at rs12457255 (SEQ ID NO: 2018), an “A” allele at rs2297437 (SEQ ID NO: 2019), a “G” allele at rs41309367 (SEQ ID NO: 2020), an “A” allele at rs10733509 (SEQ ID NO: 2021), a “G” allele at rs10750376 (SEQ ID NO: 2022), a “G” allele at rs10932456 (SEQ ID NO: 2023), an “A” allele at rs1326860 (SEQ ID NO: 2024), a “G” allele at rs1528663 (SEQ ID NO: 2025), a “C” allele at rs1892231 (SEQ ID NO: 2026), an “A” allele at rs951279 (SEQ ID NO: 2027), an “A” allele at rs9806914 (SEQ ID NO: 2028), an “A” allele at rs7935393 (SEQ ID NO: 2029), a “G” allele at rs1690492 (SEQ ID NO: 2030), an “A” allele at rs420726 (SEQ ID NO: 2031), a “T” allele at rs7759385 (SEQ ID NO: 2032), an “A” allele at rs10974900 (SEQ ID NO: 2033), an “A” allele at rs1326860 (SEQ ID NO: 2034), a “C” allele at rs2548147 (SEQ ID NO: 2035), an “A” allele at rs2815844 (SEQ ID NO: 2036), a “G” allele at rs889702 (SEQ ID NO: 2037), an “A” allele at rs9806914 (SEQ ID NO: 2038), an “A” allele at rs6478109 (SEQ ID NO: 2039), a “C” allele at rs7278257 (SEQ ID NO: 2040), an “A” allele at rs11221332 (SEQ ID NO: 2041) an “A” allele at rs56124762 (SEQ ID NO: 2057), a “G” at rs2070558 (SEQ ID NO: 2058), and a “T” allele at rs2070561 (SEQ ID NO: 2059).
59. The method of embodiments 34-58, wherein the inhibitor of TL1A activity or expression is an anti-TL1A antibody.
60. The method of embodiment 59, wherein the anti-TL1A antibody is selected from Table 20.
61. The method of embodiment 59, wherein the anti-TL1A antibody comprises an amino acid sequence provided in Tables 16-17.
62. The method of embodiment 59, wherein the anti-TL1A antibody binds to the same region of human TL1A as a reference antibody selected from Table 20.
63. The method of embodiment 59, wherein the anti-TL1A antibody binds to the same region of human TL1A as a reference antibody, the reference antibody comprising an amino acid sequence provided in Tables 16-17.
64. The method of embodiments 59-643, wherein the anti-TL1A antibody is a neutralizing TL1A antibody.
65. The method of embodiments 59-64, wherein the anti-TL1A antibody is an antagonist of TL1A.
66. A method of characterizing at least one of an inflammatory, fibrostenotic, and fibrotic, disease or condition of a subject, the method comprising assaying genetic material from the subject to identify the presence or absence of a genotype comprising a polymorphism provided in Table 1 or Table 4.
67. The method of embodiment 66, further comprising assigning a more favorable prognosis to treatment with an inhibitor of TL1A activity or expression when the genotype is present.
68. The method of embodiment 66, further comprising assigning a less favorable prognosis to with an inhibitor of TL1A activity or expression when the genotype is absent.
69. The method of embodiment 66, further comprising assigning the subject to treatment with an inhibitor of TL1A activity or expression when the genotype is present.
70. The method of embodiment 66, further comprising prescribing to the subject an inhibitor of TL1A activity or expression when the genotype is present.
71. The method of embodiment 66, further comprising administering to the subject an inhibitor of anti-CD30 ligand activity or expression when the genotype is present.
72. The method of any of embodiments 67-71, provided that the inhibitor of TL1A activity or expression is an anti-TL1A antibody or antigen-binding fragment thereof.
73. The method of any of embodiments 67-72, provided that assaying comprises amplifying from the genetic material comprising at least 15 contiguous nucleobases including a risk allele located at nucleoposition 501 within any one of SEQ ID NOS: 2001-2048, or 2057-2059 using a pair of primers comprising a first primer and a second primer.
74. The method of any of embodiment 73, provided that the first primer comprises a nucleic acid sequence complimentary to at least 15 contiguous nucleobases upstream of the risk allele located at nucleobase 501 within any one of SEQ ID NOS: 2001-2048, or 2057-2059, and the second primer comprises a nucleic acid sequence complimentary to at least 15 contiguous nucleobases downstream of the risk allele located at nucleobase501 within any one of SEQ ID NOS: 2001-2048, or 2057-2059.
75. The method of any of embodiments 66-74, provided that assaying comprises hybridizing to the genetic material a nucleic acid comprising any one of SEQ ID NOS: 2001-2048, or 2057-2059.
76. The method of embodiment 75, provided that the nucleic acid sequence is conjugated to a detectable molecule.
77. The method of embodiment 76, provided that the detectable molecule comprises a fluorophore.
78. The method of any of embodiments 75-77, provided that the nucleic acid sequence is conjugated to a quencher.
79. The method of any of embodiments 66-78, provided that assaying comprises DNA sequencing.
80. The method of any of embodiments 66-79, further comprising measuring the level of TL1A in the subject.
81. The method of any of embodiments 66-80, provided that the subject is homozygous for the genotype.
82. The method of embodiments 66-81, wherein the genotype comprises at least two polymorphisms provided in Table 1 or Table 4.
83. The method of any of embodiments 66-82, wherein the genotype comprises at least three polymorphisms provided in Table 1 or Table 4
84. The method of any one of embodiments 66-83, wherein the genotype comprises at least one polymorphism comprising a non-reference allele.
85. The method of any of embodiments 66-84, further comprising characterizing the at least one of the inflammatory, the fibrostenotic, and the fibrotic, disease or condition as Crohn's disease (CD) provided the genotype is present.
86. The method of embodiment 85, provided that the CD comprises ileal, ileocolonic, or colonic CD.
87. The method of any of embodiments 66-86, further comprising characterizing the at least one of the inflammatory, the fibrostenotic, and the fibrotic, disease or condition as a ulcerative colitis (UC), provided the genotype is present.
88. The method of embodiment 87, provided that the fibrotic disease is medically refractory UC.
89. The method of embodiment 72, wherein the anti-TL1A antibody is selected from Table 20.
90. The method of embodiment 72, wherein the anti-TL1A antibody comprises an amino acid sequence provided in Tables 16-17.
91. The method of embodiment 72, wherein the anti-TL1A antibody binds to the same region of human TL1A as a reference antibody selected from Table 20.
92. The method of embodiment 72, wherein the anti-TL1A antibody binds to the same region of human TL1A as a reference antibody, the reference antibody comprising an amino acid sequence provided in Tables 16-17.
93. The method of embodiments 89-92, wherein the anti-TL1A antibody is a neutralizing TL1A antibody.
94. The method of embodiments 89-93, wherein the anti-TL1A antibody is an antagonist of TL1A.
95. A method for detecting a genotype of interest in a subject comprising at least one of an inflammatory, a fibrostenotic, and a fibrotic, disease or condition, the method comprising:
   (a) contacting genetic material from the subject with a composition sufficiently complementary to and capable of hybridizing to the genotype of interest, the composition comprising:
      (i) a detectably labeled oligonucleotide probe comprising at least 10 contiguous nucleobases provided in any one of SEQ ID NOS: 2001-2048, or 2057-2059,
      (ii) a detectably labeled oligonucleotide probe comprising at least 10 contiguous nucleobases provided in any one of SEQ ID NOS: 2001-2048, or 2057-2059,
      (iii) a detectably labeled oligonucleotide probe comprising at least 10 contiguous nucleobases provided in any one of SEQ ID NOS: 2001-2048, or 2057-2059,
      (iv) a detectably labeled oligonucleotide probe comprising a nucleic acid sequence that differs from a probe selected from the group consisting of (i)-(iii) by up to three nucleobases, provided the detectably labeled oligonucleotide probe of (iv) hybridizes to the genotype of interest,
      (v) a detectably labeled oligonucleotide probe comprising a nucleic acid sequence complementary to a probe selected from the group consisting of (i)-(iv), or
      (vi) a combination of probes selected from the group consisting of (i)-(v), wherein the detectably labeled oligonucleotide probe of (i), (ii), and (iii) are different,
   (b) detecting the presence or absence of hybridization of the genetic material with the composition using the detectably labeled probe, whereby hybridization of the genetic material with the composition is indicative of the presence of the genotype of interest in the subject.
96. The method of embodiment 95, provided that the presence of the genotype of interest is indicative of the subject comprising elevated levels of TL1A.
97. The method of embodiment 95 or embodiment 96, provided that the inflammatory disease comprises Crohn's disease (CD).
98. The method of embodiment 97, provided that the CD comprises ileal, ileocolonic, or colonic CD.
99. The method of embodiments 99 or 92, provided that the inflammatory disease is ulcerative colitis (UC).
100. A method of treating the at least one of an inflammatory disease, a fibrostenotic disease, in the subject of any one of embodiments 95-99, the method comprising:
   j) administering to the subject of any of embodiments 95-89 a therapeutically effective amount of an inhibitor of TL1A activity or expression, provided that the subject comprises the genotype of interest.
101. The method of embodiment 100, provided that the inhibitor of TL1A activity comprises an anti-TL1A ligand antibody or antigen binding fragment thereof.
102. A composition comprising at least 10 but less than 50 contiguous nucleobase residues of any one of SEQ ID NOS: 2001-2048, or 2057-2059 or its complement, wherein the contiguous nucleobase residues comprise the nucleobase at position 501 of the any one of SEQ ID NOS: 2001-2048, or 2057-2059, and wherein the contiguous nucleobase residues are connected to a detectable molecule.
103. The composition of embodiment 102, provided that the detectable molecule is a fluorophore.
104. The composition of embodiments 102-103, wherein the contiguous nucleobase residues comprise the nucleobase at position 501 of any one of SEQ ID NOS: 2001-2048, or 2057-2059.
105. The composition of embodiments 102-104, wherein the contiguous nucleobase residues comprise the nucleobase at position 501 of any one of SEQ ID NOS: 2060-2108, or 2357-2358.
106. The composition of embodiments 102-105, provided that the contiguous nucleobase residues are connected to a quencher.
107. A kit comprising the composition of any of embodiments 102-106, and a primer pair capable of amplifying at least 15 contiguous nucleic acid molecules of any one of SEQ ID NOS: 2001-2048, or 2057-2059, the at least 15 contiguous nucleic acid molecules comprising the nucleic acid located at position 501 of any one of SEQ ID NOS: 2001-2048, or 2057-2059.
108. A method comprising contacting DNA from a subject with the composition of any of embodiments 102-106 or the kit of any of embodiment 107 under conditions configured to hybridize the composition to the DNA if the DNA comprises a sequence complementary to the composition.
109. A method comprising treating the subject of embodiment 108 with an inhibitor of TL1A activity or expression, provided that the DNA from the subject comprises the sequence complementary to the composition.
110. The method of embodiment 109, provided that the inhibitor of TL1A comprises an anti-TL1A antibody or antigen binding fragment thereof.
111. A method of identifying a risk of developing a TL1A mediated disease or condition comprising at least one of an inflammatory, a fibrostenotic, and a fibrotic, disease or condition in a subject, the method comprising:
   k) assaying a sample obtained from the subject to identify the presence of a genotype comprising a polymorphism provided in Table 1 or Table 4, or a polymorphism in linkage disequilibrium (LD) therewith; and
   l) identifying the risk of developing at least one of an inflammatory, a fibrostenotic, and a fibrotic, disease or condition in the subject, provided the presence of the genotype is identified in step (a).
112. A method of selecting a subject for treatment, the method comprising:
   m) assaying a sample obtained from the subject to identify the presence of a genotype comprising a polymorphism provided in Table 1 or Table 4, or a polymorphism in linkage disequilibrium (LD) therewith; and
   n) selecting the subject for treatment with an inhibitor of TL1A activity or expression, provided the presence of the genotype is identified in step (a).
113. The method of any of embodiments 111-112, provided that the subject is homozygous for the genotype.
114. The method of any of embodiments 111-113, wherein the genotype comprises at least two polymorphisms provided in Table 1 or Table 4.
115. The method of any of embodiments 111-114, wherein the genotype comprises at least three polymorphisms provided in Table 1 or Table 4.
116. The method of any of embodiments 111-115, wherein the genotype comprises at least four polymorphisms provided in Table 1 or Table 4.
117. The method of any of embodiments 111-116, wherein the genotype comprises at least five polymorphisms provided in Table 1 or Table 4.
118. The method of any of embodiments 111-117, wherein the genotype comprises at least six polymorphisms provided in Table 1 or Table 4.
119. The method of any of embodiments 111-118, wherein the genotype comprises at least seven polymorphisms provided in Table 1 or Table 4.
120. The method of any of embodiments 111-119, wherein the genotype comprises at least eight polymorphisms provided in Table 1 or Table 4.
121. The method of any of embodiments 111-1220, wherein the genotype comprises at least one polymorphism comprising a non-reference allele.
122. The method of embodiments 111-121, further comprising treating the subject by administering to the subject a therapeutically effective amount of an inhibitor of TL1A activity or expression.
123. The method of embodiment 122, wherein the inhibitor of TL1A activity or expression is an anti-TL1A antibody.
124. The method of embodiment 123, wherein the anti-TL1A antibody is selected from Table 20.
125. The method of embodiment 123, wherein the anti-TL1A antibody comprises an amino acid sequence provided in Tables 16-17.
126. The method of embodiment 123, wherein the anti-TL1A antibody binds to the same region of human TL1A as a reference antibody selected from Table 20.
127. The method of embodiment 123, wherein the anti-TL1A antibody binds to the same region of human TL1A as a reference antibody, the reference antibody comprising an amino acid sequence provided in Tables 16-17.
128. The method of embodiments 123-128, wherein the anti-TL1A antibody is a neutralizing TL1A antibody.
129. The method of embodiments 123-129, wherein the anti-TL1A antibody is an antagonist of TL1A.
130. The methods of embodiments 33-65 or 111-121, further comprising administering a therapeutically effective amount of an additional therapeutic agent.
131. The method of embodiment 130, wherein the additional therapeutic agent is a modulator of Receptor Interacting Serine/Threonine Kinase 2 (RIPK2).
132. The method of embodiment 130, wherein the additional therapeutic agent is a modulator of G Protein-Coupled Receptor 35 (GPR35).
133. The method of embodiment 130, wherein the additional therapeutic agent is a modulator of CD30 ligand (CD30L)
134. The method of any one of embodiments 1-134, further comprising predicting a positive therapeutic response in a subject to a treatment with the inhibitor of TL1A activity or expression with a positive predictive value of at least or about 25%, 26%, 27%, 28%, 29%, 30%, 31%, 32%, 33%, 34%, 35%, 40%, 45%, 50%, 51%, 50%, 55% 60%, 65%, 70%, 75%, 80%, 85%, 90%, 91%, 92%, 93%, 94% 95%, 96%, 97%, 98%, 99%, or 100%.
135. The method of any one of embodiments 1-135, further comprising predicting a positive therapeutic response in a subject to a treatment with the inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
136. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression, provided at least three polymorphisms comprising rs1892231, rs56124762, rs6478109, rs2070558, rs2070561, rs11897732, rs6740739, rs17796285, rs7935393, rs12934476, rs12457255, rs2070557, rs4246905, rs10974900, rs12434976, rs16901748, rs2815844, rs889702, rs2409750, rs1541020, rs4942248, rs12934476, rs12457255, rs2297437, rs41309367, rs10733509, rs10750376, rs10932456, rs1326860, rs1528663, rs951279, rs9806914, rs7935393, rs1690492, rs420726, rs7759385, rs10974900, rs1326860, rs2548147, rs2815844, rs889702, rs9806914, rs7278257, or rs11221332, or a proxy polymorphism in linkage disequilibrium therewith as determined with an R² of at least 0.85, or a combination thereof, are detected in a sample obtained from the subject; wherein the inhibitor of TL1A activity is an antibody or antigen binding fragment as described herein.
137. The method of embodiment 136, wherein the at least three polymorphisms are predictive of a positive therapeutic response in the subject to a treatment with the inhibitor of TL1A activity or expression at a positive predictive value of at least about 30%.
138. The method of embodiment 136, wherein the at least three polymorphisms are predictive of positive therapeutic response in the subject to a treatment with the inhibitor of TL1A activity or expression with a specificity of at least about 50%.
139. The method of embodiment 136, wherein the at least three polymorphisms comprise:
   (a) rs6478109, rs56124762, and rs1892231;
   (b) rs6478109, rs56124762, and rs16901748;
   (c) rs6478109, rs1892231, and rs16901748;
   (d) rs56124762, rs1892231, and rs16901748;
   (e) rs6478109, rs2070558, and rs1892231;
   (f) rs6478109, rs2070558, and rs16901748;
   (g) rs6478109, rs1892231, and rs16901748;
   (h) rs2070558, rs1892231, and rs16901748;
   (i) rs6478109, rs2070561, and rs1892231;
   (j) rs6478109, rs2070561, and rs16901748;
   (k) rs6478109, rs1892231, and rs16901748;
   (l) rs2070561, rs1892231, and rs16901748;
   (m) rs6478109, rs7935393, and rs1892231;
   (n) rs6478109, rs7935393, and rs9806914;
   (o) rs6478109, rs7935393, and rs7278257;
   (p) rs6478109, rs7935393, and rs2070557;
   (q) rs6478109, rs1892231, and rs9806914;
   (r) rs6478109, rs1892231, and rs7278257;
   (s) rs6478109, rs1892231, and rs2070557;
   (t) rs6478109, rs9806914, and rs7278257;
   (u) rs6478109, rs9806914, and rs2070557;
   (v) rs6478109, rs7278257, and rs2070557;
   (w) rs7935393, rs1892231, and rs9806914;
   (x) rs7935393, rs1892231, and rs7278257;
   (y) rs7935393, rs1892231, and rs2070557;
   (z) rs7935393, rs9806914, and rs7278257;
   (aa) rs7935393, rs9806914, and rs2070557;
   (bb) rs7935393, rs7278257, and rs2070557;
   (cc) rs1892231, rs9806914, and rs7278257;
   (dd) rs1892231, rs9806914, and rs2070557;
   (ee) rs1892231, rs7278257, and rs2070557; or
   (ff) rs9806914, rs7278257, and rs2070557.
140. The method of embodiment 136, wherein the at least three polymorphisms further comprises a fourth polymorphism comprising rs16901748, rs1892231, rs56124762, rs6478109, rs2070558, rs2070561, rs11897732, rs6740739, rs17796285, rs7935393, rs12934476, rs12457255, rs2070557, rs4246905, rs10974900, rs12434976, rs2815844, rs889702, rs2409750, rs1541020, rs4942248, rs12934476, rs12457255, rs2297437, rs41309367, rs10733509, rs10750376, rs10932456, rs1326860, rs1528663, rs951279, rs9806914, rs7935393, rs1690492, rs420726, rs7759385, rs10974900, rs1326860, rs2548147, rs2815844, rs889702, rs9806914, rs7278257, or rs11221332, or a proxy polymorphism in linkage disequilibrium therewith as determined with an R² of at least 0.85, or a combination thereof.
141. The method of method of embodiment 136, wherein the at least three polymorphisms are detected in the sample by subjecting the sample to an assay configured to detect a presence of at least three nucleotides corresponding to nucleic acid position 501 within at least three of SEQ ID NOS: 2001-2041, or 2057-2059.
142. The method of embodiment 136, wherein the at least eight polymorphisms are predictive of a positive therapeutic response in the subject to a treatment with the inhibitor of TL1A activity or expression at a positive predictive value of at least about 30%.
143. The method of embodiment 136, wherein the at least eight polymorphisms are predictive of positive therapeutic response in the subject to a treatment with the inhibitor of TL1A activity or expression with a specificity of at least about 50%.
144. The method of embodiment 136, wherein the at least eight polymorphisms comprise a set of polymorphisms selected from Table 25.
145. The method of embodiment 1, wherein the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis.
146. The method of embodiment 145, wherein the Crohn's disease is ileal, ileocolonic, or colonic Crohn's disease.
147. The method of embodiment 136, wherein the subject has, or is at risk for developing, a non-response or loss-of-response to a standard therapy comprising glucocorticosteroids, anti-TNF therapy, anti-a4-b7 therapy, anti-IL12p40 therapy, or a combination thereof.
148. The method of embodiment 136, wherein the inhibitor of TL1A is an anti-TL1A antibody or antigen-binding fragment.
149. A method of treating an inflammatory, a fibrotic, or a fibrostenotic disease or condition in a subject, the method comprising:
   (a) determining whether the subject with an inflammatory, a fibrotic, or a fibrostenotic disease or condition is suitable for treatment with an inhibitor of TL1A activity or expression by:
      (i) obtaining or having obtained a sample from the subject; and
      (ii) subjecting the sample to an assay adapted to detect at least three polymorphisms comprising rs1892231, rs56124762, rs6478109, rs2070558, rs2070561, rs11897732, rs6740739, rs17796285, rs7935393, rs12934476, rs12457255, rs2070557, rs4246905, rs10974900, rs12434976, rs16901748, rs2815844, rs889702, rs2409750, rs1541020, rs4942248, rs12934476, rs12457255, rs2297437, rs41309367, rs10733509, rs10750376, rs10932456, rs1326860, rs1528663, rs951279, rs9806914, rs7935393, rs1690492, rs420726, rs7759385, rs10974900, rs1326860, rs2548147, rs2815844, rs889702, rs9806914, rs7278257, rs11221332, or a proxy polymorphism in linkage disequilibrium therewith as determined with an R² of at least 0.85, or a combination thereof; and
   (b) treating the subject by administering a therapeutically effective amount of the inhibitor of TL1A activity or expression to the subject; wherein the inhibitor of TL1A activity or expression is an anti-TL1A antibody or antigen as described herein.
150. The method of embodiment 149, wherein the at least three polymorphisms are predictive of a positive therapeutic response in the subject to a treatment with the inhibitor of TL1A activity or expression at a positive predictive value of at least about 30%.
151. The method of embodiment 149, wherein the at least three polymorphisms are predictive of a positive therapeutic response in the subject to a treatment with the inhibitor of TL1A activity or expression at a specificity of at least about 50%.
152. A method of treating an inflammatory, a fibrotic, or a fibrostenotic disease or condition in a subject, the method comprising:
   (c) determining whether the subject with an inflammatory, a fibrotic, or a fibrostenotic disease or condition is suitable for treatment with an inhibitor of TL1A activity or expression by:
      (iii) obtaining or having obtained a sample from the subject; and
      (iv) subjecting the sample to an assay adapted to detect at least eight polymorphisms comprising rs1892231, rs56124762, rs6478109, rs2070558, rs2070561, rs11897732, rs6740739, rs17796285, rs7935393, rs12934476, rs12457255, rs2070557, rs4246905, rs10974900, rs12434976, rs16901748, rs2815844, rs889702, rs2409750, rs1541020, rs4942248, rs12934476, rs12457255, rs2297437, rs41309367, rs10733509, rs10750376, rs10932456, rs1326860, rs1528663, rs951279, rs9806914, rs7935393, rs1690492, rs420726, rs7759385, rs10974900, rs1326860, rs2548147, rs2815844, rs889702, rs9806914, rs7278257, rs11221332, or a proxy polymorphism in linkage disequilibrium therewith as determined with an R² of at least 0.85, or a combination thereof; and
   (d) treating the subject by administering a therapeutically effective amount of the inhibitor of TL1A activity or expression to the subject;
      wherein the inhibitor of TL1A activity or expression is an anti-TL1A antibody or antigen as described herein.
153. The method of embodiment 152, wherein the at least eight polymorphisms are predictive of a positive therapeutic response in the subject to a treatment with the inhibitor of TL1A activity or expression at a positive predictive value of at least about 30%.
154. The method of embodiment 152, wherein the at least eight polymorphisms are predictive of a positive therapeutic response in the subject to a treatment with the inhibitor of TL1A activity or expression at a specificity of at least about 50%.
155. The method of embodiment 136, wherein the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis.
156. The method of embodiment 156, wherein the Crohn's disease is ileal, ileocolonic, or colonic Crohn's disease.
157. The method of embodiment 149, wherein the wherein the inhibitor of TL1A activity or expression is an anti-TL1A antibody or antigen-binding fragment.
158. The method of embodiment 149, wherein the at least three polymorphisms comprise:
   (a) rs6478109, rs56124762, and rs1892231;
   (b) rs6478109, rs56124762, and rs16901748;
   (c) rs6478109, rs1892231, and rs16901748;
   (d) rs56124762, rs1892231, and rs16901748;
   (e) rs6478109, rs2070558, and rs1892231;
   (f) rs6478109, rs2070558, and rs16901748;
   (g) rs6478109, rs1892231, and rs16901748;
   (h) rs2070558, rs1892231, and rs16901748;
   (i) rs6478109, rs2070561, and rs1892231;
   (j) rs6478109, rs2070561, and rs16901748;
   (k) rs6478109, rs1892231, and rs16901748;
   (l) rs2070561, rs1892231, and rs16901748;
   (m) rs6478109, rs7935393, and rs1892231;
   (n) rs6478109, rs7935393, and rs9806914;
   (o) rs6478109, rs7935393, and rs7278257;
   (p) rs6478109, rs7935393, and rs2070557;
   (q) rs6478109, rs1892231, and rs9806914;
   (r) rs6478109, rs1892231, and rs7278257;
   (s) rs6478109, rs1892231, and rs2070557;
   (t) rs6478109, rs9806914, and rs7278257;
   (u) rs6478109, rs9806914, and rs2070557;
   (v) rs6478109, rs7278257, and rs2070557;
   (w) rs7935393, rs1892231, and rs9806914;
   (x) rs7935393, rs1892231, and rs7278257;
   (y) rs7935393, rs1892231, and rs2070557;
   (z) rs7935393, rs9806914, and rs7278257;
   (aa) rs7935393, rs9806914, and rs2070557;
   (bb) rs7935393, rs7278257, and rs2070557;
   (cc) rs1892231, rs9806914, and rs7278257;
   (dd) rs1892231, rs9806914, and rs2070557;
   (ee) rs1892231, rs7278257, and rs2070557; or
   (ff) rs9806914, rs7278257, and rs2070557.
159. The method of embodiment 136, wherein the at least three polymorphisms further comprises a fourth polymorphism comprising rs16901748, rs1892231, rs56124762, rs6478109, rs2070558, rs2070561, rs11897732, rs6740739, rs17796285, rs7935393, rs12934476, rs12457255, rs2070557, rs4246905, rs10974900, rs12434976, rs2815844, rs889702, rs2409750, rs1541020, rs4942248, rs12934476, rs12457255, rs2297437, rs41309367, rs10733509, rs10750376, rs10932456, rs1326860, rs1528663, rs951279, rs9806914, rs7935393, rs1690492, rs420726, rs7759385, rs10974900, rs1326860, rs2548147, rs2815844, rs889702, rs9806914, rs7278257, or rs11221332 or a proxy polymorphism in linkage disequilibrium therewith as determined with an R² of at least 0.85, or a combination thereof.
160. The method of embodiment 136, wherein the subject is at risk of developing a non-response or loss-of-response to a standard therapy comprising glucocorticosteroids, anti-TNF therapy, anti-a4-b7 therapy, anti-IL12p40 therapy, or a combination thereof.
161. A method of treating an inflammatory, a fibrotic, or a fibrostenotic disease or condition in a subject, the method comprising administering to the subject a therapeutically effective amount of an inhibitor of TL1A activity or expression, wherein the subject expresses at least three polymorphisms comprising rs16901748, rs6478109, rs56124762, or a proxy polymorphism in linkage disequilibrium therewith as determined with an R² of at least 0.85;
   wherein the inhibitor of TL1A activity of expression is an nti-TL1A antibody or antigen-binding fragment as described herein.
162. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, wherein said antibody or antigen-binding fragment comprises:
      o) a heavy chain comprising a heavy chain complementarity determining region 1 (HCDR1), a heavy chain complementarity determining region 2 (HCDR2), and a heavy chain complementarity determining region 3 (HCDR3), wherein the HCDR1 comprises a first amino acid sequence of DTYMH of SEQ ID NO: 601; the HCDR2 comprises a second amino acid sequence of PASGH of SEQ ID NO: 768; and the HCDR3 comprises a third amino acid sequence of SGGLPD of SEQ ID NO: 805; and
      p) a light chain comprising a light chain complementarity determining region 1 (LCDR1), a light chain complementarity determining region 2 (LCDR2), and a light chain complementarity determining region 3 (LCDR3), wherein the LCDR1 comprises a fourth amino acid sequence of ASSSVSYMY of SEQ ID NO: 851; the LCDR2 comprises a fifth amino acid sequence of ATSNLAS of SEQ ID NO: 11; and the LCDR3 comprises a sixth amino acid sequence of GNPRT of SEQ ID NO: 921.
163. The method of embodiment 163, provided that the antibody or antigen-binding fragment is a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof.
164. The method of embodiment 163, provided that the antibody or antigen-binding fragment is a humanized antibody.
165. The method of embodiment 163, where the antibody or antigen-binding fragment of embodiment 141 is administered with a pharmaceutically acceptable carrier.
166. The method of embodiment 163, provided the antibody or antigen binding fragment is an immunoglobulin G (IgG).
167. The method of embodiment 165, provided the IgG comprises an IgG1.
168. The method of embodiment 164, provided the IgG comprises an IgG2.
169. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, wherein said antibody or antigen-binding fragment comprises an antibody or antigen binding fragment thereof that binds to TL1A, comprising a heavy chain variable region comprising complementarity determining regions (CDRs) as set forth in SEQ ID NOS: 3001, 3002, and 3006; and a light chain variable region comprising CDRs as set forth in SEQ ID NOS: 3010, 3011, and 3013.
170. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least eight polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, wherein said antibody or antigen-binding fragment comprises an antibody or antigen binding fragment thereof that binds to TL1A, comprising a heavy chain variable region comprising complementarity determining regions (CDRs) as set forth in SEQ ID NOS: 3001, 3002, and 3006; and a light chain variable region comprising CDRs as set forth in SEQ ID NOS: 3010, 3011, and 3013.
171. The method of embodiment 170 or 171, wherein the light chain variable region comprises SEQ ID NO: 3204.
172. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3005, and 3008; and a light chain variable region comprising CDRs as set forth in SEQ ID NOS: 3010, 3011, and 3012.
173. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least eight polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3005, and 3008; and a light chain variable region comprising CDRs as set forth in SEQ ID NOS: 3010, 3011, and 3012.
174. The method of embodiment 173 or 174, wherein the light chain variable region comprises SEQ ID NO: 3202.
175. The method of any one of embodiments 173-175, wherein the heavy chain variable region comprises SEQ ID NO: 3121.
176. The method of any one of embodiments 173-175, wherein the heavy chain variable region comprises SEQ ID NO: 3122.
177. The method of any one of embodiments 173-175, wherein the heavy chain variable region comprises SEQ ID NO: 3123.
178. The method of any one of embodiments 173-175, wherein the heavy chain variable region comprises SEQ ID NO: 3124.
179. The method of embodiments 173 or 174, wherein the light chain variable region comprises SEQ ID NO: 3205.
180. The method of embodiment 173 or embodiment 174, wherein the heavy chain variable region comprises SEQ ID NO: 3122.
181. The method of embodiment 173 or embodiment 174, wherein the heavy chain variable region comprises SEQ ID NO: 3124.
182. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3005, and 3008; and a light chain variable region comprising CDRs as set forth in SEQ ID NOS: 3010, 3011, and 3013.
183. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least eight polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3005, and 3008; and a light chain variable region comprising CDRs as set forth in SEQ ID NOS: 3010, 3011, and 3013.
184. The method of embodiment 183 or 184, wherein the heavy chain variable region comprises SEQ ID NO: 3122.
185. The method of embodiment 183 or embodiment 184, wherein the light chain variable region comprises SEQ ID NO: 3204.
186. The method of embodiment 183 or embodiment 184, wherein the light chain variable region comprises SEQ ID NO: 3206.
187. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3003, and 3008; and a light chain variable region comprising CDRs as set forth in SEQ ID NOS: 3010, 3011, and 3013.
188. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least eight polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable region comprising complementarity determining regions (CDRs) as set forth in SEQ ID NOS: 3001, 3003, and 3008; and a light chain variable region comprising CDRs as set forth in SEQ ID NOS: 3010, 3011, and 3013.
189. The method of embodiment 188 or 189, wherein the heavy chain variable region comprises SEQ ID NO: 3128.
190. The method of embodiment 188 or 189, wherein the light chain variable region comprises SEQ ID NO: 3206.
191. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least three polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework.
192. A method of treating an inflammatory, a fibrotic, or a fibrostenotic disease or condition in a subject, the method comprising administering to the subject a therapeutically effective amount of an inhibitor of Tumor necrosis factor-like cytokine 1A (TL1A) activity or expression,
   wherein at least eight polymorphisms that are predictive of a positive therapeutic response in the subject to a treatment with the inhibitor of TL1A activity or expression with a positive predictive value or a specificity of at least about 30%, are detected in a sample obtained from the subject, and wherein the inhibitor of TL1A activity or expression comprises an antibody or antigen-binding fragment that specifically binds to a tumor necrosis factor-like protein 1A (TL1A) polypeptide, comprising a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework.
193. The method of embodiment 193 or 194, wherein the heavy chain variable framework region and the light chain variable framework region collectively comprise 13, 12, 11, 10, 9, 8, 7, 6, 5, 4, 3, 2, 1, or no amino acid modifications from the human IGHV1-46*02 framework and the human IGKV3-20 framework.
194. The method of any one of embodiments 170-193, wherein the antibody is humanized.
195. The method of any one of embodiments 170-194, wherein the antibody comprises a human IgG1 fragment crystallizable (Fc) region.
196. The method of any one of embodiments 170-194, wherein the antibody comprises a human IgG4 fragment crystallizable (Fc) region.
197. The method of embodiments 170-197, wherein the disease or condition comprises inflammatory bowel disease.
198. The method of embodiments 170-198, wherein the disease or condition comprises Crohn's disease.
199. The method of embodiments 170-199, wherein the disease or condition comprises ulcerative colitis.
200. The method of any one of embodiments 170-200, further comprising predicting a positive therapeutic response in a subject to a treatment with the inhibitor of TL1A activity or expression with a positive predictive value of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
201. The method of any one of embodiments 170-201, further comprising predicting a positive therapeutic response in a subject to a treatment with the inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.

In some embodiments, the at least three polymorphisms is eight polymorphisms. Non-limiting examples of eight polymorphism combinations are provided in Table 25 (495 combinations). In some embodiments, the at least three polymorphisms is any combination of polymorphisms selected from Table 31.

### 5.7 Kits

Further provided is a kit to treat IBD (e.g., CD, UC and/or mrUC). The kit comprises of the antibodies described herein, which can be used to perform the methods described herein. The kit is useful for practicing the inventive method of providing treatment to an IBD, CD, UC and/or mrUC patient by administering an anti-TL1A antibody. The kit is an assemblage of materials or components, including at least one of the inventive compositions. Thus, in some embodiments, the kit contains a composition including anti-TL1A antibodies, for the treatment of IBD, CD, UC and/or MR-UC, as described above. In other embodiments, the kits contain all of the components necessary and/or sufficient to perform a detection assay for TL1A, including all controls, directions for performing assays, and any necessary software for analysis and presentation of results.

The exact nature of the components configured in the inventive kit depends on its intended purpose. For example, some embodiments are configured for the purpose of treating IBD, CD, UC and/or MR-UC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or alleviate IBD, CD, UC and/or MR-UC. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of an inventive composition containing anti-TL1A antibodies and/or primers and probes for TL1A. The packaging material generally has an external label which indicates the contents and/or purpose of the kit and/or its components.

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject suitable for treatment with an inhibitor of TL1A activity or expression (e.g., anti-TL1A antibody). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. An exemplary therapeutic agent is an anti-TL1A antibody.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### 5.8 Systems

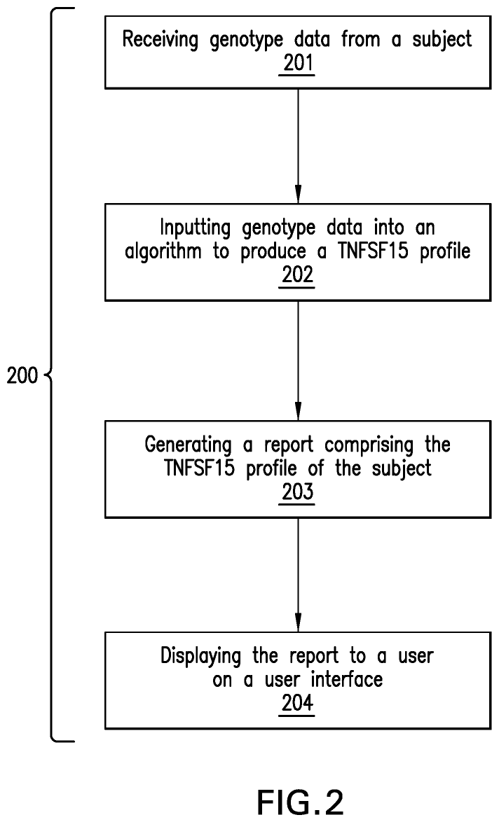

Disclosed herein are systems for treating a subject with an inhibitor of TL1A activity or expression (e.g., anti-TL1A antibody. In some embodiments, the systems described herein comprise kits and compositions for detecting the genotypes described herein in a biological sample of a subject. The system may comprise a computer system for implementing one or more methods of the disclosure, such as for example, receiving genotype data of a subject 201, inputting the genotype data into an algorithm to produce a TNFSF15 profile 202, and generating a report comprising the TNFSF15 profile of the subject 203, and displaying the report to a user on a graphical user interface 204, as shown in FIG. 2. A “TNFSF15 profile” as used herein refers to a profile of one or more genotypes described herein of a subject that is detected in a biological sample obtained from the subject. In some embodiments, a TNFSF15 profile comprises a positive, a negative, or an indeterminate result (e.g., therapeutic response to treatment with an inhibitor of TL1A activity or expression).

### 5.8.1 Computer Systems

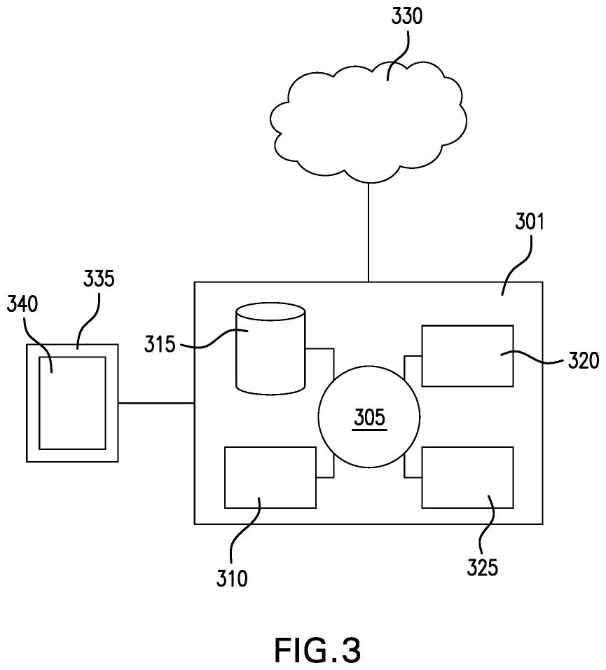

FIG. 3 shows a computer system 301 that is programmed or otherwise configured to generate a TNFSF15 profile for a subject in need thereof. The computer system 301 can regulate various aspects of producing the TNFSF15 profile (e.g., receiving genotype data, generating a report with the TNFSF15 profile of the biological sample, and displaying the report to a user), of the present disclosure, such as, for example, by including permissions or encryption of genotype data and/or TNFSF15 profile of the subject to ensure patient privacy.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. In some embodiments, the software comprises the Assay Interpretation Software, as disclosed elsewhere herein. In some embodiments, the software comprises the Model Identification Software as disclosed elsewhere herein. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the TNFSF15 profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface. In some embodiments, the computer system comprises one or more input devices. Non-limiting examples of input devices comprise a computer mouse, keyboard, microphone, trackball, scanner, joystick, camera, or any other suitable input device.

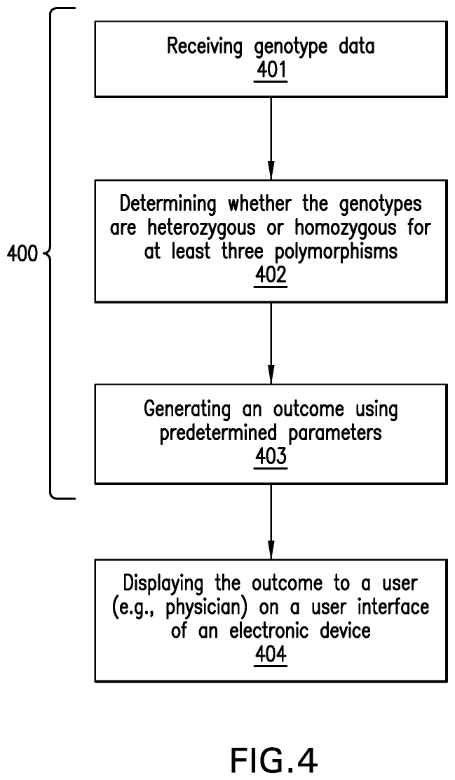

Methods and systems of the present disclosure can be implemented by way of one or more algorithms. An algorithm can be implemented by way of software upon execution by the central processing unit 305. The algorithm can, for example, perform: (a) receiving genotype data of a subject 401, (b) determining whether the genotypes are heterozygous or homozygous for at least three polymorphisms 402, (c) generating an outcome using predetermined parameters 403, and (d) displaying the outcome to a user (e.g., physician) on a user interface of an electronic device 404, as shown in FIG. 4. In some embodiments, the outcome is positive, negative or indeterminant. In some embodiments, the predetermined parameters are genotype combinations known to be predictive of a therapeutic response to a treatment, such as with an inhibitor of TL1A activity or expression.

### 5.8.2 Web Application

In some embodiments, the computer system comprises software for a web application. In light of the disclosure provided herein, those of skill in the art will recognize that a web application may utilize one or more software frameworks and one or more database systems. A web application, for example, is created upon a software framework such as Microsoft® NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### 5.8.3 Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, R, SQL, MATLAB, Scala, Julia, SAS, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

### 5.8.4 Standalone Application

In some embodiments, the computer system comprises software a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB NET, or combinations thereof. Compilation may be often performed, at least in part, to create an executable program. In some instances, a computer program includes one or more executable complied applications.

### 5.8.5 Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### 5.8.5.1 Model Identification Software

Disclosed herein is model identification software that employs a machine learning approach to identify single nucleotide polymorphisms (SNP) models that can be used to predict a positive therapeutic response to an inhibitor of TL1A activity or expression. In some embodiments, a polygenetic risk score (“PRS”) is calculated for genetic loci associated with inflammatory bowel disease (e.g., Crohn's disease, ulcerative colitis) that are involved in the TL1A pathway. There are many suitable methods of calculating a PRS, including but not limited to the methodologies provided in Li et al., 2018. Inflamm Bowel Dis. 12; 24(11):2413-2422, which is hereby incorporated by reference. In some embodiments, a ≤25th percentile of the PRS distribution indicates a low likelihood of developing the inflammatory bowel disease or severe form of the disease. In some embodiments, a ≥75^{th} percentile of the PRS distribution indicates a high likelihood of developing the inflammatory bowel disease or severe form of the disease.

A data set from several genome wide association (GWA) studies for subjects with inflammatory bowel disease or severe forms of the disease may be analyzed using a gradient boosting algorithm that applies the distribution of PRS across the dataset to assign an output value to subset of the dataset that are indicative of ≤25th percentile of the PRS distribution and ≥75^{th} percentile of the PRS distribution. Non-limiting examples of gradient boosting algorithms include XGBoost (Extreme Gradient Boosting), AdaBoost algorithm (Adaptive Boosting), gradient boosting algorithm, Gradient Tree Boosting algorithm, LightGBM (Light Gradient Boosting Machine), or CatBoost. In some embodiments, a similar analysis may be performed to cluster samples obtained from patients with the inflammatory bowel disease based on gene expression using a soft clustering algorithm. Non-limiting examples of soft clustering algorithms include mixed-effects models with nonparametric smoothing spline fitting as implemented in TMixClust and Fuzzy C-Means Clustering. In some embodiments, the gene expression is quantified by measuring protein expression. In some embodiments, the gene expression is quantified by measuring RNA expression, such as mRNA). In some embodiments, the expression is expression of TL1A protein or mRNA. In some embodiments, the distribution comprises two or more clusters, such as for example high producers of the RNA or protein (cluster 1) and low producers of the RNA or protein (cluster 2). The resulting SNPs common to both analyses (analyses using the gradient boosting algorithm and the soft clustering algorithms) are therefore predictive of high RNA or protein expression in patients with inflammatory bowel disease, and predictive of a PRS in the ≥75^{th} percentile.

A Market Based Analysis may be performed to determine a combination of rules to predict genotype combinations that are predictive of high RNA or protein expression in patients with inflammatory bowel disease, and predictive of a PRS in the ≥75^{th} percentile. Such a Market Based Analysis is described in Breuer et al. Int J Bipolar Disord. 2018; 6:24), which is herein incorporated by reference. In some embodiments, a support vector machine (SVM) and regression may be used to prioritize the SNPs based on the SVM or regression coefficient.

In some embodiments, the model identification software comprises one or more algorithms disclosed herein. In some embodiments the one or more algorithms comprises a classifier configured to classify samples by assigning output values. In some embodiments, the output values comprise descriptive labels, numerical values, or a combination thereof. In some embodiments, the algorithm is a linear classifier. In some embodiments, the linear classifier algorithm is a logistic regression. In some embodiments, the linear classifier algorithm is Naive Bayes classifier. In some embodiments, the linear classifier algorithm is Fisher's linear discriminant. In some embodiments, the algorithm is support vector machine. In some embodiments, the support vector machine algorithm is least squares support vector machines. In some embodiments, the algorithm is quadratic classifier. In some embodiments, the algorithm is Kernel estimation. In some embodiments, the Kernel estimation algorithm is k-nearest neighbor. In some embodiments, the algorithm is a decision tree. In some embodiments, the decision tree algorithm is random forest. In some embodiments, the algorithm is neural network. In some embodiments, the algorithm is learning vector quantization.

Some of the output values may comprise descriptive labels. Such descriptive labels may provide an identification or indication of the positive output or a negative output. In some embodiments, the positive output comprises ≥75^{th} percentile of the PRS distribution. In some embodiments, the positive output comprises high expression of a protein or RNA (e.g., TL1A). In some embodiments, the negative output comprises ≤25th percentile of the PRS distribution. In some embodiments, the negative output comprises low expression of a protein or RNA (e.g., TL1A). In some embodiments, the output values comprise a relative output, such as a high-risk, an intermediate-risk, or a low-risk, which is relative to an index or a control. In some embodiments, the index or the control is derived from reference subjects that have responded to a treatment with an inhibitor of TL1A activity or expression (positive control), and/or reference subjects that have not responded to a treatment with an inhibitor of TL1A activity or expression (negative control).

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject is in the ≥75^{th} percentile of the PRS distribution. As another example, a binary classification of samples may assign an output value of “negative” or 0 if the sample indicates that the subject is the ≤25th percentile of the PRS distribution. In this case, a single cutoff value of 25^{th} and 75^{th} percentiles is used to classify samples into one of the two possible binary output values or classes of individuals.

In another example, a binary classification of samples may assign an output value of “positive” or 1 if the sample indicates that the subject is a high producer of gene expression (e.g., TL1A). As another example, a binary classification of samples may assign an output value of “negative” or 0 if the sample indicates that the subject is a low producer of gene expression. In some embodiments, a high producer is relative to a cutoff value of mean value, median value, slope of TL1A expression time curve of at least 10 degree, slope of TL1A expression time curve of at least 10 degree, slope of TL1A expression time curve of at least 15 degree, slope of TL1A expression time curve of at least 20 degree, slope of TL1A expression time curve of at least 25 degree, slope of TL1A expression time curve of at least 30 degree, slope of TL1A expression time curve of at least 35 degree, slope of TL1A expression time curve of at least 40 degree, slope of TL1A expression time curve of at least 10 degree, slope of TL1A expression time curve of at least 45 degree, or slope of TL1A expression time curve of at least 50 degree. In some embodiments, a low producer is relative to a cutoff value of mean value, median value, or slope of TL1A expression time curve of 10 degree. In some embodiments the cutoff value

The classifier may be configured to classify samples by assigning output values based on one or more cutoff values. For example, a binary classification of samples may assign an output value of “positive” or 1 if the sample indicates that the subject at least a 50% probability of responding to an inhibitor of TL1A activity or expression, thereby assigning the subject to a class of individuals receiving a positive result. As another example, a binary classification of samples may assign an output value of “negative” or 0 if the sample indicates that the subject has less than 50% probability of responding to an inhibitor of TL1A activity or expression, thereby assigning the subject to a class of individuals receiving a negative test result. In this case, a single cutoff value of 50% is used to classify samples into one of the two possible binary output values or classes of individuals. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of responding to an inhibitor of TL1A activity or expression of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of responding to an inhibitor of TL1A activity or expression of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of responding to an inhibitor of TL1A activity or expression of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of responding to an inhibitor of TL1A activity or expression of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of responding to an inhibitor of TL1A activity or expression. Examples of sets of cutoff values may include {1%, 99%}{2%, 98%}, {5%, 95%}{10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

In some embodiments, the SNP models are predictive of a positive therapeutic response to an inhibitor of TL1A activity or expression with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of predicting a positive therapeutic response to an inhibitor of TL1A activity or expression using the algorithm may be calculated as the percentage of samples identified or classified as responding to an inhibitor of TL1A activity or expression that correspond to subjects that truly responded to an inhibitor of TL1A activity or expression.

In some embodiments, the SNP models are predictive of a positive therapeutic response to an inhibitor of TL1A activity or expression with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of predicting a positive therapeutic response to an inhibitor of TL1A activity or expression using the algorithm may be calculated as the percentage of samples identified or classified as having a positive therapeutic response to an inhibitor of TL1A activity or expression that correspond to subjects that truly responded to an inhibitor of TL1A activity or expression.

In some embodiments, the SNP models are predictive of a positive therapeutic response to an inhibitor of TL1A activity or expression with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of predicting a positive therapeutic response to an inhibitor of TL1A activity or expression using the algorithm may be calculated as the percentage of independent test samples associated with a positive therapeutic response to an inhibitor of TL1A activity or expression (e.g., subjects known to have responded to the inhibitor of TL1A activity or expression) that are correctly identified or classified as having a positive therapeutic response to an inhibitor of TL1A activity or expression.

In some embodiments, the SNP models are predictive of a positive therapeutic response to an inhibitor of TL1A activity or expression with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of predicting a positive therapeutic response to an inhibitor of TL1A activity or expression using the algorithm may be calculated as the percentage of independent test samples associated with absence of a positive therapeutic response to an inhibitor of TL1A activity or expression (e.g., subjects with negative clinical test results for response to an inhibitor of TL1A activity or expression) that are correctly identified or classified as not having a positive therapeutic response to an inhibitor of TL1A activity or expression.

In some embodiments, the SNP models identify the presence (e.g., positive test result) or absence (e.g., negative test result) of a positive therapeutic response to an inhibitor of TL1A activity or expression with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the algorithm in classifying samples as having or not having a positive therapeutic response to an inhibitor of TL1A activity or expression. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

The one or more algorithms in the model identification software may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the positive therapeutic response to an inhibitor of TL1A activity or expression. The classifiers may be adjusted or tuned by adjusting parameters of the classifiers (e.g., a set of cutoff values used to classify a sample as described elsewhere herein) to improve or optimize the performance metrics. The one or more classifiers may be adjusted or tuned so as to reduce an overall classification error.

### 5.8.5.2 Assay Interpretation Software

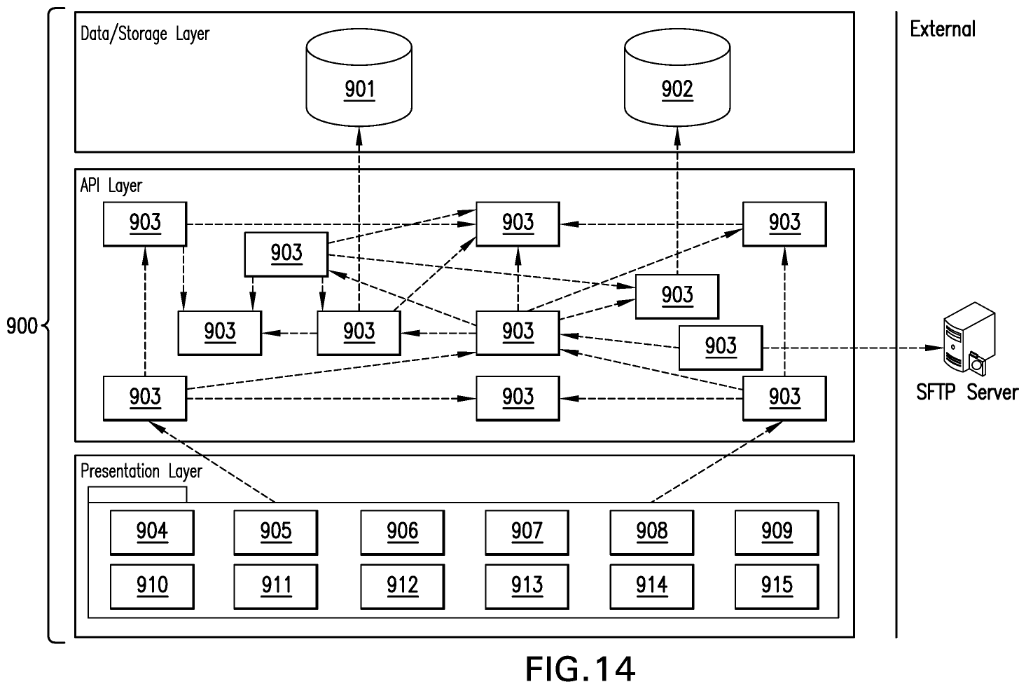

In some embodiments, the software modules described herein comprise genotype analysis software modules or display software modules, or combinations thereof as shown in FIG. 14. In some embodiments, the genotype analysis software modules are communicatively coupled to each other. In some embodiments, the genotype analysis software modules are configured to receive data, analyze data, and/or transmit data to other genotype analysis software modules within the API layer, or to one or more databases 901, 902 in the data/storage layer. In some embodiments, the genotype analysis software modules 903 are communicatively coupled to one or more databases described herein. Display software modules disclosed herein within the display layer enable the display of a result from the genotype analysis performed by the genotype analysis software modules. In some embodiments, the display software modules are implemented as a web application, a mobile application, or a standalone application.

The genotype analysis software modules instruct the one or more computer processors to output a result for a sample. In some embodiments, the result is positive, which indicates that the subject from which the sample was obtained is predicted to respond to an inhibitor of TL1A activity or expression (e.g., an anti-TL1A antibody or antigen binding fragment thereof). In some embodiments, the result is negative, which indicates that the subject from which the sample was obtained is not predicted to respond to the inhibitor of TL1A activity or expression. In some embodiments, the result is indeterminate.

In some embodiments, the genotype analysis software module comprises one or more algorithms configured to instruct one or more processers to perform the methods disclosed herein (e.g., analyze genotype data to produce a TL1A profile). In some embodiments, the one or more algorithms comprises a Brute Force algorithm. In some embodiments, the one or more algorithms comprises a Greedy algorithm. In some embodiments, the one or more algorithms comprises a Recursive algorithm. In some embodiments, the one or more algorithms comprises a Backtracking algorithm. In some embodiments, the one or more algorithms comprises a Divide & Conquer algorithm, In some embodiments, the one or more algorithms comprises a Dynamic programming algorithm. In some embodiments, the one or more algorithms comprises a Randomized algorithm. In some embodiments, the genotype analysis software module comprises a script of a plurality of such algorithms that is configured to analyze genotype data in a genotype file disclosed herein.

In some embodiments, the algorithm is configured to detect genotype combinations at select single nucleotide polymorphism (SNP) positions of the one or more models disclosed herein. In the case of a 3-SNP model, the algorithm may analyze the genotypes in two or three SNP positions (a possible 27 genotype combinations), and compares the genotype combinations that are detected in the sample with a series of genotype combinations of the 3-SNP model that has been determined to be associated with a positive result as described elsewhere herein. If the algorithm determines a presence of one or more of the series of genotypes associated with a positive result, then a positive result is assigned to the sample. If the algorithm determines an absence of one or more of the series of genotypes associated with a positive result, then a negative result is assigned to the sample. In some embodiments, the algorithm is redirected to a positive result without analyzing the entire genotype data set upon detection of one or more genotype combinations disclosed herein that has been determined to have an association with predicting a positive therapeutic response to the inhibitor of TL1A activity or expression that outweighs the association of the remaining possible genotypes combinations that could be detected.

In some embodiments of the methods provided herein, including in Sections 2, 5 (including in the paragraphs of Section 5.8.5), and 7, determining a positive result comprises (1) determining a positive result when the PRI is above a cutoff if the PRI has a positive correlation with Response Probability Score (RPS); or (2) determining a positive result when the PRI is below a cutoff if the PRI has a negative correlation with RPS, wherein the PRI is calculated from a combination of genotypes determined from a sample from the subject. In some embodiments of the methods provided herein, including in Sections 2, 5 (including in the paragraphs of Section 5.8.5), and 7, determining a positive result comprises (1) determining a positive result when the PRI is above a cutoff if the PRI has a positive correlation with Model Risk Score (MRS); or (2) determining a positive result when the PRI is below a cutoff if the PRI has a negative correlation with MRS, wherein the PRI is calculated from a combination of genotypes determined from a sample from the subject. In some embodiments of the methods provided herein, including in Sections 2, 5 (including in the paragraphs of Section 5.8.5), and 7, determining a positive result comprises determining a positive result when the RPS is above a cutoff, wherein the RPS is calculated from a combination of genotypes determined from a sample from the subject. In some embodiments of the methods provided herein, including in Sections 2, 5 (including in the paragraphs of Section 5.8.5), and 7, determining a positive result comprises determining a positive result when the MRS is above a cutoff, wherein the MRS is calculated from a combination of genotypes determined from a sample from the subject.

In some embodiments, each genotype is assigned a numerical value and weights are applied to those values based on the significance of the SNP in predicting a positive therapeutic response to the inhibitor of TL1A activity or expression. For example, a genotype may be assigned the number “0” for two copies of the minor allele, a “1” for one copy of the major allele and one copy of the mino allele, or a “2” for two copies of the major allele. Other examples of numeric encoding of genotypes or polymorphisms are described in Table 28. In some embodiments, the algorithm calculates the sum of the numerical values it assigns for each SNP in the model. In the case of a 3-SNP model as described elsewhere herein, the algorithm would calculate a score between 0 and 6. In some embodiments, the algorithm weights each SNP based on its relative significance in predicting a positive therapeutic response to the inhibitor of TL1A activity or expression, by giving SNPs with less significance a smaller weight than those SNPs having a larger significance. For example a particular SNP may be given a weight of 1.5, meaning that the possible score would be 0, 1.5, or 9, if its significance in predicting positive therapeutic response to the inhibitor of TL1A activity or expression is higher than the others in the model. In some embodiments, the weight comprises multiplier of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0 or 5.0.

In some embodiments, one or more genotype data analysis software modules executes a script to analyze the genotypes in the genotype file. A MRS and/or RPS score is assigned as described in Examples, using the SNP models described in Table 31. MRS can be used to determine the CDx result (positive or negative) by comparing the MRS against the cutoff as described in the definition of MRS. Similarly, RPS can be used to determine the CDx result (positive or negative) by comparing the RPS against the cutoff as described in the definition of RPS and elsewhere in this disclosure.

In some embodiments of the various methods and systems provided herein including in Sections 2, 5 (including but not limited to Sections 5.8.1, 5.8.5, 5.8.11, and 5.8.12) and 7, the TL1A profile comprises or consists of RPS. In some embodiments of the various methods and systems provided herein including in Sections 2, 5 (including but not limited to Sections 5.8.1, 5.8.5, 5.8.11, and 5.8.12) and 7, the TL1A profile comprises or consists of MRS score. In some embodiments of the various methods and systems provided herein including in Sections 2, 5 (including but not limited to Sections 5.8.1, 5.8.5, 5.8.11, and 5.8.12) and 7, the TL1A profile comprises or consists of PRI. In some embodiments of the various methods and systems provided herein including in Sections 2, 5 (including but not limited to Sections 5.8.1, 5.8.5, 5.8.11, and 5.8.12) and 7, the TL1A profile comprises or consists of the combination of the polymorphisms of a subject. In some embodiments of the various methods and systems provided herein including in Sections 2, 5 (including but not limited to Sections 5.8.1, 5.8.5, 5.8.11, and 5.8.12) and 7, the TL1A profile comprises or consists of the genotypes of the combination of the polymorphisms.

### 5.8.5.3 Assay Interpretation Software Architecture

The software architecture described herein may be a layered architecture pattern, wherein the individual layers (e.g., horizontal layers) may be interconnected. In some embodiments, the software architecture is provided in FIG. 14, which illustrates three horizontal layers in the architecture. In some embodiments, the layered architecture pattern comprises 2, 3, 4, 5, 6, 7, 8, 9, or 10 layers. In some embodiments, the layered architecture pattern comprises between 2 and 10, 3 and 9, 4 and 9, or 5 and 7 layers. In some embodiments, the layered architecture pattern is a hybrid layered architecture, wherein additional layers are added or existing layers are modified. In some embodiments, one or more of the layers is optional (or “open”). In some embodiments, one or more of the layers is required (or “closed”). In some embodiments, all of the layers in the layered architecture pattern are closed. In some embodiments, each layer comprises similar software modules or “components,” such that each layer is relatively isolated from the others to perform a set of related tasks. In some embodiments, each component is interconnected to at least one other component within the same layer, and optionally connected with components in different layers. In some embodiments, the software architecture is a central computing environment, in which a majority of the computing is performed by a central processor. In some embodiments, the software architecture is a Distributed Computing Environment (DCE).

Referring to FIG. 14, the layered architecture pattern may comprise a data/storage layer, an API layer, or a presentation layer, or any combination thereof. In some embodiments, the data/storage layer comprises one or more databases 901, 902. The one or more databases is configured to store data, including by not limited to the genotype data, one or more TL1A profile(s) of a subject, clinical study samples, algorithms, metadata, and the like). In some embodiments, the API layer comprises genotype analysis software modules 903. In some embodiments, the genotype analysis software modules 903 are custom rules or algorithms that handle the exchange of information between the one or more databases and a user interface disclosed herein. In some embodiments, the genotype analysis software modules 903 instruct the one or more processors of the computer system to analyze the genotype data disclosed herein and generate a TL1A profile for a subject. In some embodiments, the presentation layer comprises display software modules 904-915. In some embodiments, the presentation layer comprises components related to the presentation of information related to the genotype analysis to a user (e.g., genotype data (input), the TL1A profile (output) for a sample). In some embodiments, the display software modules comprises a login view 904, a files view 905, a samples view 906, a data analyses view 907, a new data analysis view 908, an algorithm editor view 909, a system settings view 910, a clinical settings view 911, a clinical study settings view 912, a user management view 913, an activities view 914, or a my profile view 915, or any combination thereof.

In some embodiments, the genotype analysis software modules 903 are communicatively coupled to each other. In some embodiments, the genotype analysis software modules 903 are configured to receive data, analyze data, and/or transmit data to other genotype analysis software modules 903 within the API layer, or to one or more databases 901, 902 in the data/storage layer. In some embodiments, the genotype analysis software modules 903 are communicatively coupled to one or more databases 901, 902 described herein. Display software modules 904-915 disclosed herein within the display layer enable the display of a result from the genotype analysis performed by the genotype analysis software modules 903. In some embodiments, the display software modules are implemented as a web application, a mobile application, or a standalone application.

In some embodiments, the layered architecture pattern comprises one or more additional layers comprising a service layer, or a persistence layer, or any combination thereof.

### 5.8.6 Databases

The medium, method, and system disclosed herein comprise one or more databases, or use of the same. In view of the disclosure provided herein, those of skill in the art will recognize that many databases are suitable for storage and retrieval of genotype data, a TL1A profile of a subject, clinical study data user preference and login information, and the like. Suitable databases include, by way of non-limiting examples, relational databases, non-relational databases, object oriented databases, feature databases, entity-relationship model databases, associative databases, XML databases, document oriented databases, and graph databases. Further non-limiting examples include SQL, PostgreSQL, MySQL, Oracle, DB2, Sybase, and MongoDB.

Referring to Assay Interpretation Software architecture provide in FIG. 14, in some embodiments, the computer systems described herein comprise one or more databases 901, 902. In some embodiments, the software modules 903 are communicatively coupled to the one or more databases 901, 902. In some embodiments, the one or more database 901, 902 is internet-based. In some embodiments, the one or more databases 901, 902 is web-based. In some embodiments, the one or more databases 901, 902 is cloud computing-based. In some embodiments, the one or more databases 901, 902 may be based on one or more local computer storage devices.

### 5.8.7 Data Transmission

The subject matter described herein, including methods for producing a TNFSF15 profile are configured to be performed in one or more facilities at one or more locations. Facility locations are not limited by country and include any country or territory. In some instances, one or more steps are performed in a different country than another step of the method. For example, referring to FIG. 2, the step of receiving genotype data from a subject 201, inputting the genotype data into the algorithm to produce a TL1A profile 202, and generating a repot comprising the TL1A profile of the subject 203 may be performed in a different country or territory than displaying the report to the user on the user interface 204.

In some instances, one or more steps for obtaining a biological sample are performed in a different country than one or more steps for analyzing the biological sample, such as detecting the presence or absence of a genotype in the biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein (e.g., genotype data). For example, referring to FIG. 1, the step of providing a buccal swab sample from the subject 101 may be performed at a facility or location that is separate from the sample processing facility where the step of assaying the sample to detect genotypes of at least two or at least three genetic loci 103 is performed. In some embodiments, the step of processing the genotypes to produce the TL1A profile 104 is performed a data processing facility that may be separate from the sample processing facility. In some embodiments of the methods and systems described herein, the analysis is performed and a subsequent data transmission step will convey or transmit the results of the analysis.

As shown in FIG. 14, in some embodiments, the results of the analysis are transmitted from one or more software modules 903 in the API layer to one or more software modules in the presentation layer, which enable a user to view the results in one or more views described herein. In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### 5.8.8 User

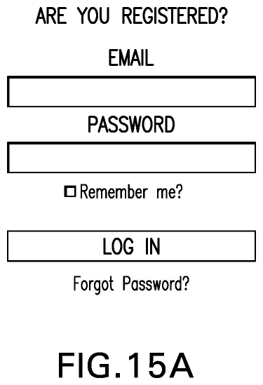

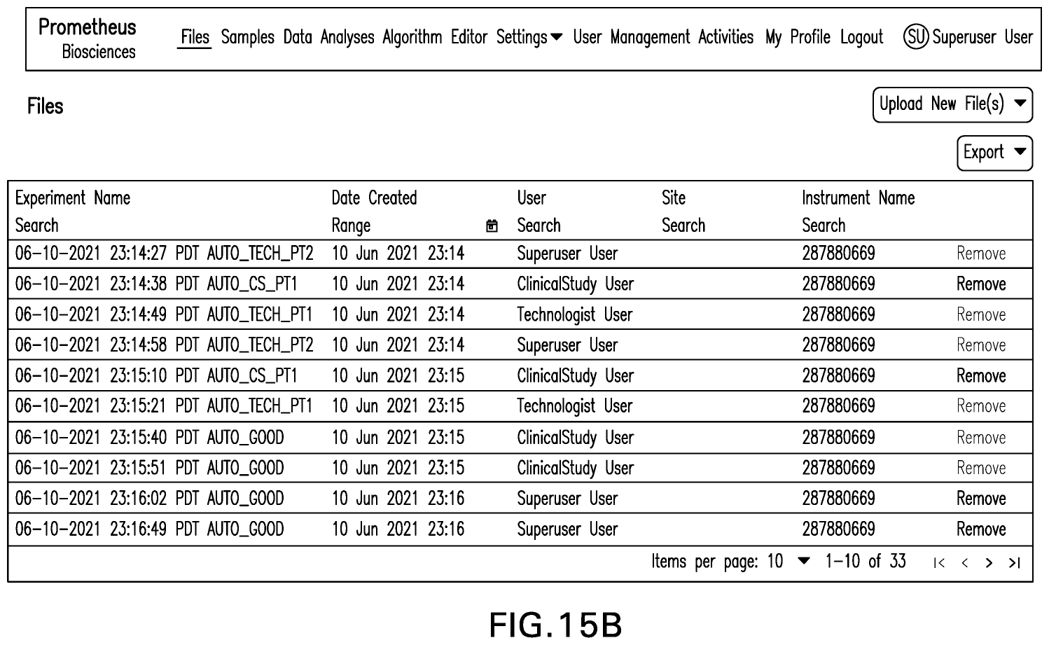

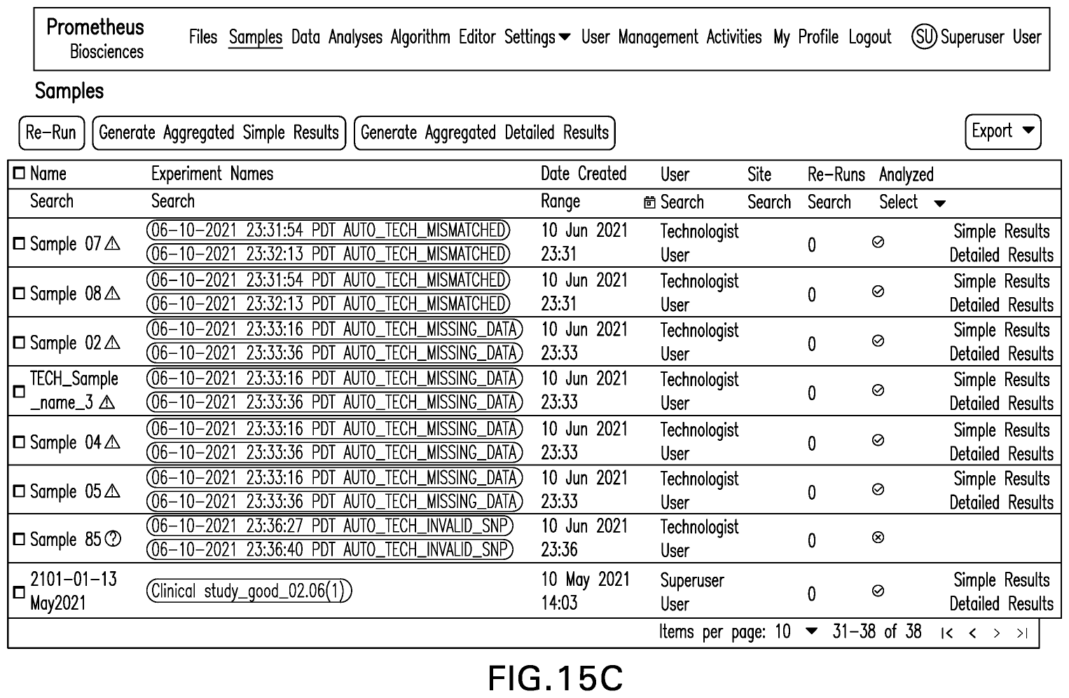

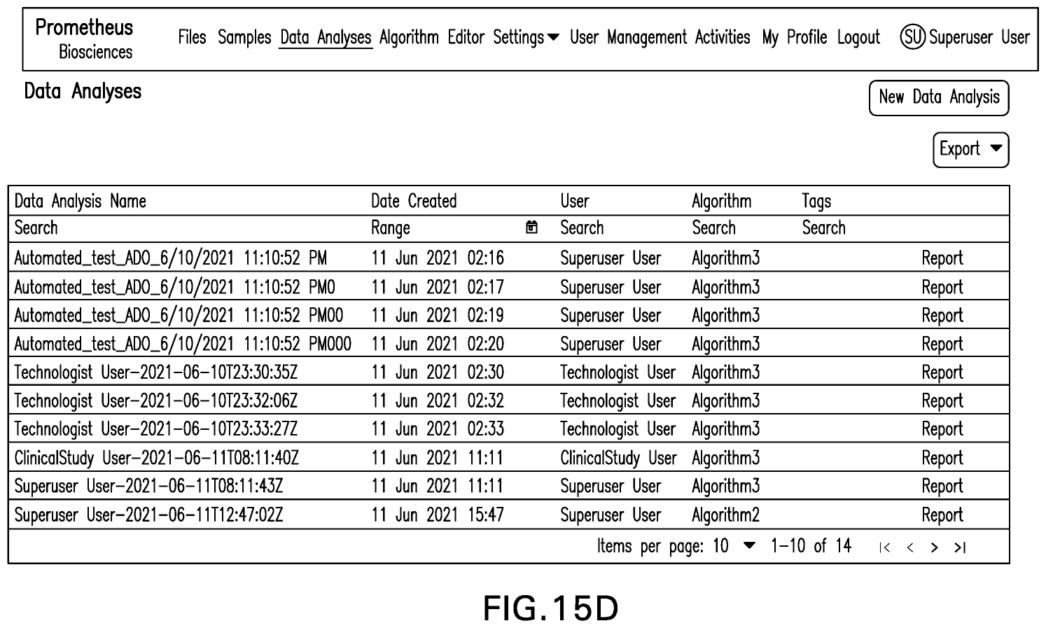

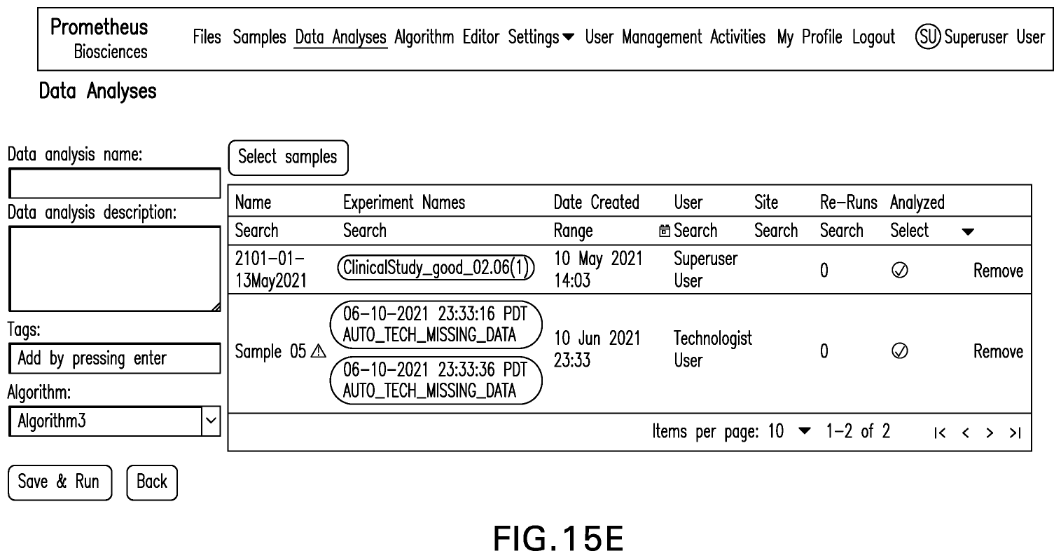

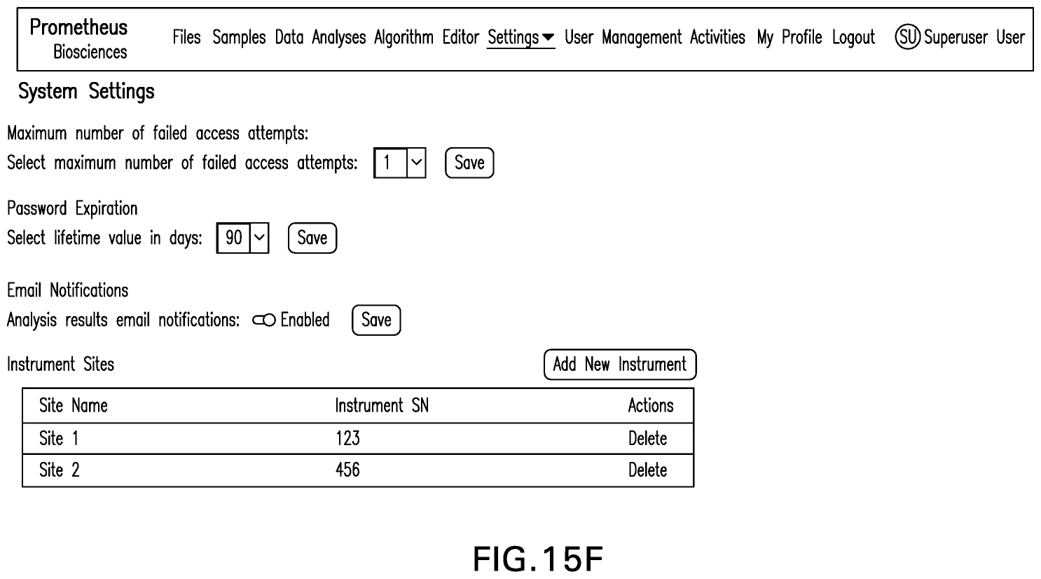

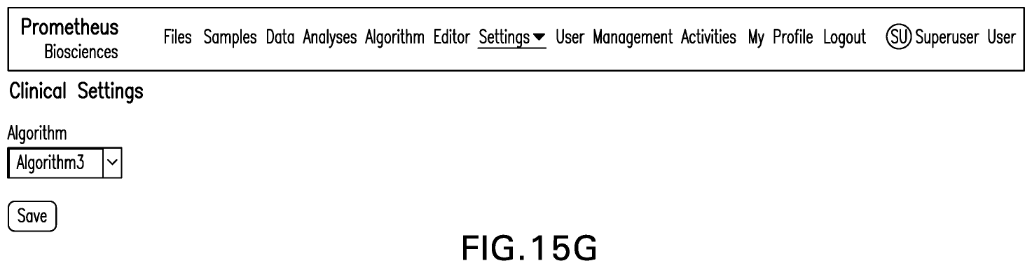

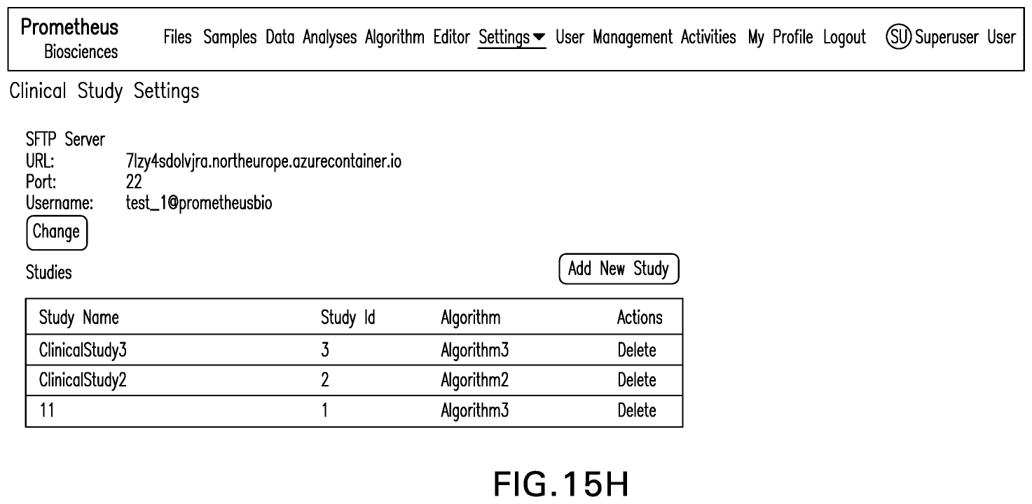

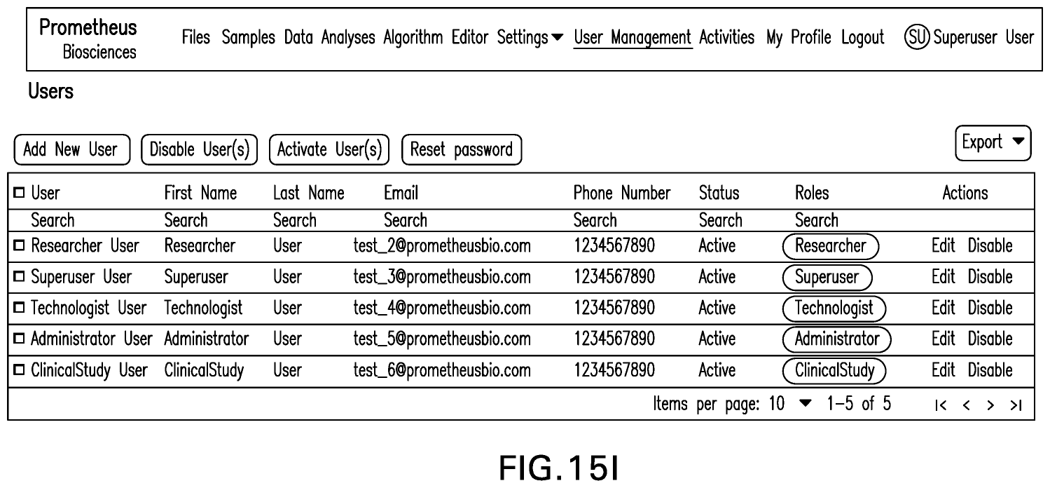

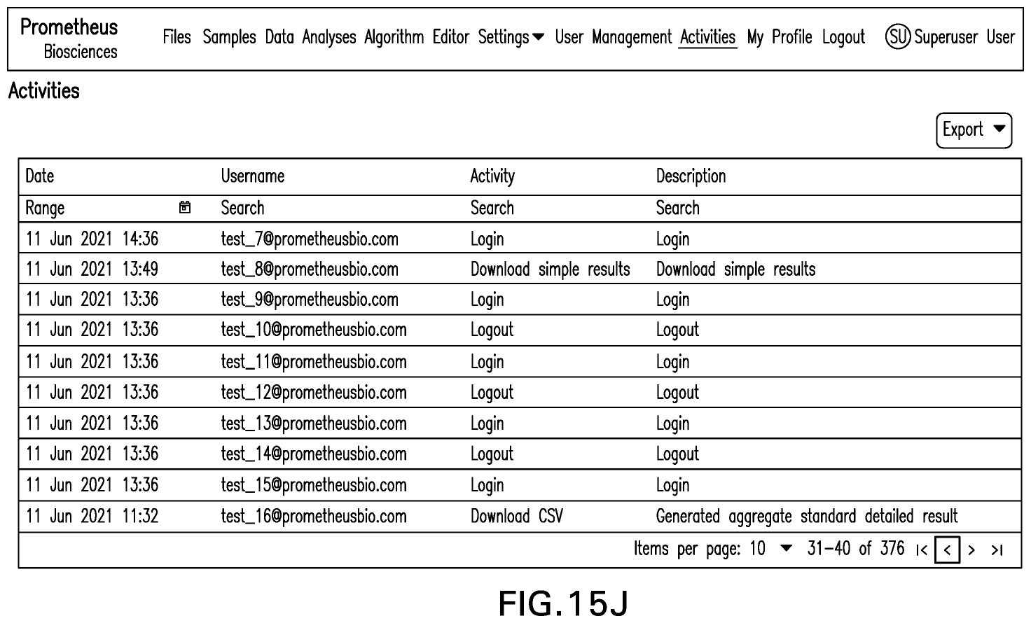

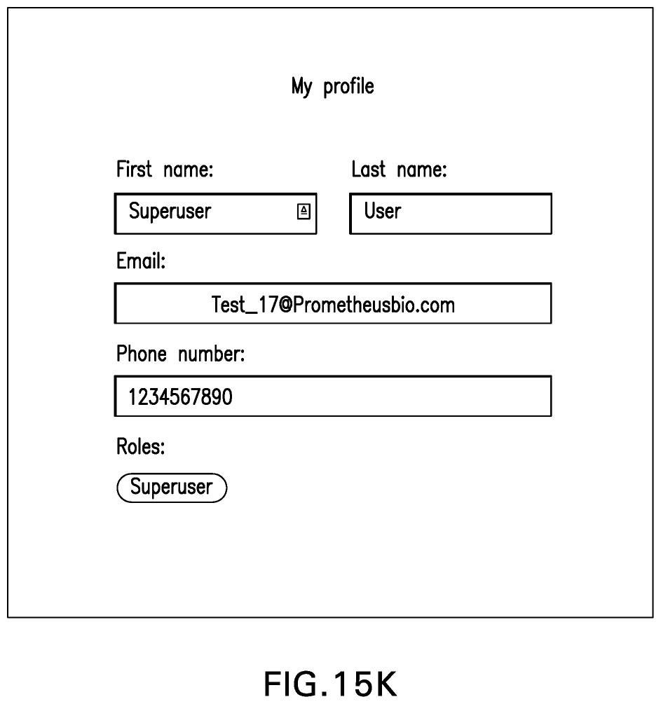

The users of the computer systems disclosed herein may have different privileges when using the software programs disclosed herein. In some embodiments, the user comprises a super user, an administrator, a researcher, a clinical technician, or a technologies, as shown in FIG. 15I. In some embodiments, the design of the software is such that certain software modules (genotype analysis software modules, display software modules) are inaccessible to certain users.

### 5.8.9 User Interface

The computer systems disclosed herein, in some embodiments, comprise a user interface for displaying information related to an analysis, such as a result to a user. Referring to FIG. 14, one or more display software modules 904-915 in the display layer instructs the one or more computer processors to display information related to an analysis of a sample to the user. In some embodiments, the information related to ana analysis of a sample comprises the genotype data (uploaded), samples analyzed, algorithm(s) used, clinical settings, or results from the analyses performed. In some embodiments, the one or more display software modules runs as a web application, a standalone application, or a mobile application as disclosed herein. In some embodiments, the one or more display software modules in the display layer comprises a login view 904, a files view 905, a samples view 906, a data analyses view 907, a new data analysis view 908, an algorithm editor view 909, a system settings view 910, a clinical settings view 911, a clinical study settings view 912, a user management view 913, an activities view 914, or a my profile view 915, or any combination thereof.

In some embodiments, the login view 904 is provided in FIG. 15A. In some embodiments, the user logs in as a registered user to a portal and uploads a data file comprising genotype data for analysis by the genotype analysis software modules 903. In some embodiments, the genotype data comprises the two alleles present at each polymorphism location in a biological sample. In the case of one single nucleotide polymorphism, the genotype is one of the following maximum possible genotypes: (i) one copy of the major allele and one copy of the minor allele, (ii) two copies of the minor allele, and (iii) two copies of the major allele. In some embodiments, the minor allele is a variant allele. In the case of analyzing three single nucleotide polymorphisms, the maximum number of possible genotypes is 27, calculated by multiplying three (3) by itself three (3) times (3\hat{ }3). In some embodiments, the genotype data is numerical, where a “1” is assigned for one copy of the major allele and one copy of the minor allele, a “0” is assigned for two copies of the minor allele, and “2” is assigned for two copies of the major allele. In some embodiments, the data or the data file further comprises (i) the patient identification (ID), (ii) disease type, (iii) collection site ID, (iv) physician ID, (v) both the patient ID and disease type, (vi) both the patient ID and collection site ID, (vii) both the patient ID and physician ID, (viii) both disease type and collection side ID, (ix) both disease type and physician ID, (x) both collection side ID and physician ID, (xi) the patient ID, disease type and collection side ID, (xii) the patient ID, disease type, and physician ID, (xiii) the patient ID, collection side ID, and physician ID, (xiv) disease type, collection side ID, and physician ID, or (xv) the patient ID, disease type, collection side ID, and physician ID.

In some embodiments, the files view 905 is provided in FIG. 15B. In the files view 905, the user can evaluate whether the samples from within the files are valid and match the required number of polymorphisms. If the samples are not valid, then the samples may be removed from the analysis. If the samples are valid, then the analysis of the genotypes of those samples will begin. In some embodiments, a warning flag is provided by the software if a sample has a mismatch duplicate (e.g. one replicate is called a “1” and other replicate is called a “2”, one replicate is called a 0 and the other called 1, one replicate called a 0 and the other called a 2), an invalid call, an invalid SNP (e.g. incorrect SNP name or SNP genotypes not recognizable by the software), or a duplicate call missing (e.g. if a replicate is required to make a call and the replicate does not have data). In some embodiments, the software allows overrise of certain SNPs, such as, for example, by prompting the user to upload another data file that contains the rest of the SNPs that were not present in the previous data file.

In some embodiments, the samples view 906 is provided in FIG. 15C. in the samples view 906, the user may rerun the analysis of the samples, generate a report to display the results, or export the table content. In some embodiments, the sample view provides to the user one or more of the name of the sample, the experiment name, the date created, the user (e.g., the user that uploaded the first sample information), the site (site location of the instrument that created the genotype data file), reruns (number of reruns performed for the selected sample), analyzed (true/false if the sample was analyzed), or a simple result from the genotype data analysis. In some embodiments, the genotype data file was created by a polymerase chain reaction (PCR) instrument, such as a real-time PCT instrument. In some embodiments, the genotype file is a .FASTQ file created from a next generation sequencing instrument.

In some embodiments, the data analyses view 907 is provided in FIG. 15D. The data analysis view 907 may only be viewed by certain uses, such as those with additional privileges (e.g., super user and researcher). In this view, the user can create a new analysis with custom configurations, search and filter the results, select a different algorithm for a rerun of the samples, and export a version of the report that contain additional details. In some embodiments, the new data analysis view 908 is provided in FIG. 15E. The new data analysis view 908 is used by super users or researches to configure and run custom analyses.

In some embodiments, the algorithm editor view 909 allows superusers and researches to create a new algorithm from scratch, or modify an existing algorithm, such as to optimize the desired number of polymorphisms or types of polymorphisms to detect in a sample to provide a positive or a negative result.

In some embodiments, the system settings view 910 is provided in FIG. 15F. The system settings view 910 allows superusers and administrators to change the configurations of the account settings (e.g., failed login attempts, password lifetime expiration).

In some embodiments, the clinical settings view 911 is provided in FIG. 15G. The clinical settings view 911 permits superusers to change the default algorithms for certain samples. In some embodiments, the clinical study settings view 912 is provided in FIG. 15H. The clinical study setting view permits superusers to change the default algorithms used for certain samples depending on the study they came from.

In some embodiments, the user management view 913 is provided in FIG. 15I. The user management view 913 permits superusers and administrators to manage the account by adding new users to the platform, activate or disable certain users, and the like. In some embodiments, the activities view 914 is provided in FIG. 15J. The activities view 914 allows any user to trace the activities of other uses, as well as sort, filter, and/or export the activities in a viewable format. In some embodiments, a my profile view 915 is provided in FIG. 15K. The my profile view 915 can be accessed by each user in the system.

### 5.8.10 Reports

In some embodiments, the results from the analyses described herein (to produce a TL1A profile) are provided to a user in a report. In some embodiments, the reports comprise the result (e.g., positive or negative) for a sample of a subject. In some embodiments, a positive result indicates that the subject is predicted to have a positive therapeutic response to an inhibitor of TL1A activity or expression. In some embodiments, a negative result indicates that the subject is not predicted to have a positive therapeutic response to the inhibitor of TL1A activity or expression. In some embodiments, the report further comprises the date created, the user (e.g., the user that uploaded the first sample information), the site (site location of the instrument that created the genotype data file), reruns (number of reruns performed for the selected sample), or analyzed (true/false if the sample was analyzed), or any combination thereof.

In some embodiments of the methods provided herein, including in Sections 2, 5 (including in the paragraphs of Section 5.8.10), and 7, determining a positive result comprises (1) determining a positive result when the PRI is above a cutoff if the PRI has a positive correlation with Response Probability Score (RPS); or (2) determining a positive result when the PRI is below a cutoff if the PRI has a negative correlation with RPS, wherein the PRI is calculated from a combination of genotypes determined from a sample from the subject. In some embodiments of the methods provided herein, including in Sections 2, 5 (including in the paragraphs of Section 5.8.10), and 7, determining a positive result comprises (1) determining a positive result when the PRI is above a cutoff if the PRI has a positive correlation with Model Risk Score (MRS); or (2) determining a positive result when the PRI is below a cutoff if the PRI has a negative correlation with MRS, wherein the PRI is calculated from a combination of genotypes determined from a sample from the subject. In some embodiments of the methods provided herein, including in Sections 2, 5 (including in the paragraphs of Section 5.8.10), and 7, determining a positive result comprises determining a positive result when the RPS is above a cutoff, wherein the RPS is calculated from a combination of genotypes determined from a sample from the subject. In some embodiments of the methods provided herein, including in Sections 2, 5 (including in the paragraphs of Section 5.8.10), and 7, determining a positive result comprises determining a positive result when the MRS is above a cutoff, wherein the MRS is calculated from a combination of genotypes determined from a sample from the subject.

### 5.8.11 Computer-Implemented Methods

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the TNFSF15 profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### 5.8.12 Systems Embodiments

Among the exemplary embodiments are:
1. A computer system for evaluating a sample from a subject, the system comprising:
   a) a computing environment;
   b) an input device operatively connected to said computing environment, wherein said input device is configured to receive a combination of genotype determined from the sample, wherein the combination of genotypes is predictive of a positive therapeutic response to an inhibitor of TL1A activity or expression;
   c) an algorithm executed by one or more processors in said computing environment, wherein the algorithm is configured to use the combination of genotypes to calculate a Predictive Response Index (PRI) and classify said sample as from a subject having a positive therapeutic response to the inhibitor of TL1A activity or expression, wherein the classification is based on PRI as follows:
      (i) if PRI has a positive correlation with RPS, then classify said sample as from a subject having a positive therapeutic response to the inhibitor of TL1A activity or expression if PRI is above a cutoff; or
      (ii) if PRI has a negative correlation with RPS, then classify said sample as from a subject having a positive therapeutic response to the inhibitor of TL1A activity or expression if PRI is below a cutoff; and
   d) an output device operatively connected to said computing environment, wherein said output device is configured to provide information on the classification to a user.
2. A computer system for evaluating a sample from a subject, the system comprising:
   a) a computing environment;
   b) an input device operatively connected to said computing environment, wherein said input device is configured to receive a combination of genotype determined from the sample, wherein the combination of genotypes is predictive of a positive therapeutic response to an inhibitor of TL1A activity or expression;
   c) an algorithm executed by one or more processors in said computing environment, wherein the algorithm is configured to use the combination of genotypes to calculate a Predictive Response Index (PRI) and classify said sample as from a subject having a positive therapeutic response to the inhibitor of TL1A activity or expression, wherein the classification is based on PRI as follows:
      (i) if PRI has a positive correlation with MRS, then classify said sample as from a subject having a positive therapeutic response to the inhibitor of TL1A activity or expression if PRI is above a cutoff; or
      (ii) if PRI has a negative correlation with MRS, then classify said sample as from a subject having a positive therapeutic response to the inhibitor of TL1A activity or expression if PRI is below a cutoff; and
   d) an output device operatively connected to said computing environment, wherein said output device is configured to provide information on the classification to a user.
3. The computer system of embodiment 1 or 2, wherein the subject has a disease comprising at least one of an inflammatory, a fibrostenotic, and a fibrotic, disease or condition.
4. The computer system of embodiment 3, wherein the disease is a TL1A mediated disease state selected from the group consisting of inflammatory bowel disease (IBD), Crohn's disease (CD), obstructive CD, ulcerative colitis (UC), intestinal fibrosis, intestinal fibrostenosis, rheumatoid arthritis, and primary sclerosing cholangitis.
5. The computer system of any previous embodiment, wherein the sample comprises or consists of whole blood, plasma, serum, or tissue.
6. The computer system of any previous embodiment, wherein the combination of genotypes comprises genotypes at at least one polymorphism selected from Table 1, Table 4, or Table 27, a polymorphism in linkage disequilibrium (LD) therewith, and any combination thereof.
7. The computer system of any previous embodiment, wherein the combination of genotypes comprises genotypes at at least one polymorphism comprising a non-reference allele.
8. The computer system of any previous embodiment, wherein the combination of genotypes comprises genotypes at at least two, three, four, five, six, seven, or eight polymorphisms provided in Table 1, Table 4, or Table 27.
9. The computer system of any preceding embodiment, wherein said algorithm is configured to classify said sample as a positive therapeutic response to said inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
10. The computer system of any preceding embodiment, wherein said algorithm is configured to classify said sample as said positive therapeutic response to said inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
11. The computer system of any preceding embodiment, wherein said algorithm is configured to classify said sample as a positive therapeutic response to said inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
12. The computer system of any preceding embodiment, wherein said algorithm is configured to classify said sample as a positive therapeutic response to an inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
13. The computer system of any preceding embodiment, wherein the computing environment comprises a central computing environment or a distributed computing environment.
14. The computer system of any previous embodiment, wherein the combination of genotypes comprises a homozygous genotype at at one or more polymorphism position.
15. The computer system of embodiment 5-13, where LD is defined by an r² value of at least 0.80, 0.85, 0.90, 0.95, or 1.0.
16. The computer system of any previous embodiment, wherein the combination of genotypes is associated with a risk that a subject has, or will have positive therapeutic response to the inhibitor of TL1A activity or expression by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸, about 1.0×10⁻⁹⁰, or about 1.0×10⁻¹⁰⁰.
17. The computer system of any previous embodiment, wherein said output device provides a report summarizing said information on said classification.
18. The computer system of any previous embodiment, wherein said report comprises a recommendation for treatment of said disease of the subject.
19. The computer system of embodiment 18, wherein the treatment comprises administration of an inhibitor of TL1A activity or expression.
20. The computer system of embodiment 19, wherein the inhibitor of TL1A activity or expression comprises an antibody or antigen-binding fragment, peptide, or small molecule.
21. The computer system of any preceding embodiment, wherein said combination of genotypes is determined with an assay comprising polymerase chain reaction (PCR), quantitative reverse-transcription PCR (qPCR), automated sequencing, genotype array, or a combination thereof.
22. Use of a composition comprising one or more binding agents for generating a report that classifies a sample from a subject as at least one of (i) a disease or non-disease state and (ii) a response or a non-response to an anti-TL1A therapy, wherein the one or more binding agents specifically bind to a risk allele provided in Table 1, Table 4, or Table 27 corresponding to a polymorphism provided in Table 1, Table 4, or Table 27 their compliment, a polymorphism in linkage disequilibrium therewith, and any combination thereof, wherein the classification is based on a PRI calculated from the polymorphism as follows:
   (i) if PRI has a positive correlation with MRS, then classify said sample as from a subject having a positive therapeutic response to the inhibitor of TL1A activity or expression if PRI is above a cutoff; or
   (ii) if PRI has a negative correlation with MRS, then classify said sample as from a subject having a positive therapeutic response to the inhibitor of TL1A activity or expression if PRI is below a cutoff.
23. The use of embodiment 22, wherein generating the report further comprises:
   a) providing the sample from the subject;
   b) assaying the sample from the subject for detecting the presence of a combination of polymorphisms provided in Table 1, Table 4, or Table 27;
   c) generating the report based on the result of step (b), wherein the classification is based on the PRI calculated from the combination of polymorphisms; and
   d) determining whether said subject has or is likely to exhibit a positive therapeutic response to a treatment with an inhibitor of TL1A activity or expression based on the results of step (b).
24. The use of embodiment 22 or 23, wherein the subject has a disease comprising at least one of an inflammatory, a fibrostenotic, and a fibrotic, disease or condition.
25. The use of embodiment 22-24, wherein the disease state is a TL1A-mediated disease state selected from the group consisting of inflammatory bowel disease (TBD), Crohn's disease (CD), obstructive CD, ulcerative colitis (UC), intestinal fibrosis, intestinal fibrostenosis, and primary sclerosing cholangitis.
26. The use of any of embodiments 22-25, wherein the sample comprises or consists of whole blood, plasma, serum, or tissue.
27. The use of any of embodiments 23-26, wherein assaying the sample from the subject for detecting the presence of the risk allele corresponding to the polymorphism provided in Table 1, Table 4, or Table 27 of step (b) comprises:
   (i) contacting the sample with the one or more binding agents that specifically bind to at least 10 contiguous nucleobases that includes the risk allele provided in any one of SEQ ID NOS: 2001-2041, or 2057-2059; and
   (ii) determining whether the sample specifically binds to said one or more binding agents, wherein binding of the sample to the one or more binding agents indicates the presence of the polymorphism in the subject.
28. The use of any of embodiments 23-27, wherein assaying the sample from the subject for detecting the presence of the risk allele corresponding to the polymorphism provided in Table 1, Table 4, or Table 27 of step (b) comprises sequencing the sample.
29. The use of any of embodiments 23-28, wherein assaying the sample from the subject for detecting the presence of the one or more polymorphisms of step (b) comprises quantifying the amount of DNA comprising the risk allele.
30. The use of embodiment 29, wherein the quantifying comprises PCR.
31. The use of embodiment 30, wherein the PCR comprises real-time PCR.
32. The use of embodiment 29, wherein the quantifying comprises hybridization.
33. The use of any one of embodiments 23-32, wherein said report classifies said sample as a positive therapeutic response to said inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
34. The use of any one of embodiments 23-33, wherein said report classifies said sample as said positive therapeutic response to said inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%, 35. The use of any one of embodiments 23-34, wherein said report classifies said sample as said positive therapeutic response to said inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
36. The use of any one of embodiments 23-35, wherein said report classifies said sample as said positive therapeutic response to said inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
37. The use of any one of embodiments 23-36, wherein the report further comprises a date the report was created, the user that generated the report, the location of a genotype device used to create a genotype data file was produced, a number or reruns for said sample, whether said sample was analyzed, or any combination thereof.
38. A composition comprising one or more binding agents that specifically bind to a risk allele corresponding to a polymorphism provided in Table 1, Table 4 or Table 27, wherein the one or more binding agents are selected to classify a sample as at least one of (i) a disease or non-disease or a disease state and (ii) a response or a non-response to an inhibitor of TL1A activity or expression, wherein the classification is based on a PRI calculated from the polymorphism as follows:
   (i) if PRI has a positive correlation with MRS, then classify said sample as from a subject having a positive therapeutic response to the inhibitor of TL1A activity or expression if PRI is above a cutoff; or
   (ii) if PRI has a negative correlation with MRS, then classify said sample as from a subject having a positive therapeutic response to the inhibitor of TL1A activity or expression if PRI is below a cutoff.
39. The composition of embodiment 38, wherein the one or more binding agents comprise oligonucleotides.
40. The composition of embodiment 39, wherein the oligonucleotides comprise RNA or DNA.
41. The composition of embodiment 40, wherein the one or more binding agents comprise aptamers, antibodies, peptide nucleic acids, or pyranosyl RNA.
42. The composition of any one of embodiments 38-41, wherein one or more binding agents bind to risk alleles corresponding to a combination of polymorphisms provided in Table 1, Table 4, or Table 27.
43. The composition of any one of embodiments 38-42, wherein said one or more binding agents classify said sample as a positive therapeutic response to said inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
44. The composition of any one of embodiments 38-43, wherein said one or more binding agents classify said sample as said positive therapeutic response to said inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
45. The composition of any one of embodiments 38-44, wherein said one or more binding agents classify said sample as said positive therapeutic response to said inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
46. The composition of any one of embodiments 38-45, wherein said one or more binding agents classify said sample as said positive therapeutic response to said inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
47. A kit for detecting at least one of an inflammatory, a fibrostenotic, and a fibrotic, disease or condition in a subject, the kit comprising:
   e) at least one binding agent that specifically binds to at least 10 contiguous nucleic acid molecules provided in any one of SEQ ID NOS: 2001-2041, or 2057-2059 including a corresponding risk allele provided in Table 1, or their complement, wherein the at least one binding agent is selected to detect at least one of (i) a disease or non-disease state and (ii) a response or a non-response to an anti-TL1A therapy; and
   f) reagents for detecting binding of said at least one binding agent to a DNA sample from a subject.
48. The kit of embodiment 47, wherein the at least one binding agent comprises at least one oligonucleotide.
49. The kit of embodiment 47, wherein the at least one binding agent comprises at least one aptamer, antibody, peptide nucleic acid, or pyranosyl RNA.
50. The kit of any one of embodiments 47-49, wherein the at least one binding agent is labelled with a detectable label.
51. The kit of any one of embodiments 47-50, wherein the at least one binding agent is immobilized to a surface.
52. A system for generating a report that classifies a sample as from a subject having a positive therapeutic response to the inhibitor of TL1A activity or expression, comprising:
   g) a computer system comprising one or more computer processors individually or collectively programmed to:
      i. generates a molecular profile of a DNA sample from a subject based upon the presence of a combination of genotypes at 2 or more polymorphism positions corresponding to a combination of polymorphisms provided in Table 1, Table 4 or Table 27, or a polymorphism in linkage disequilibrium (LD) therewith; and
      ii. generate the report that classifies the sample of the subject based on said molecular profile, wherein the classification is based on PRI calculated from the combination of genotypes as follows:
         (1) if PRI has a positive correlation with MRS or RPS, then classify said sample as from the subject having a positive therapeutic response to the inhibitor of TL1A activity or expression if PRI is above a cutoff; or
         (2) if PRI has a negative correlation with MRS or RPS, then classify said sample as from the subject having a positive therapeutic response to the inhibitor of TL1A activity or expression if PRI is below a cutoff; and
   h) a user interface configured to display said report to a user.
53. The system of embodiment 52, wherein the presence of the at least one polymorphism is based on the result of an assay of said DNA sample provided in a genotype data file that is analysed by the one or more computer processors.
54. The system of any one of embodiments 52-53, further comprising said genotype data file.
55. The system of any one of embodiments 52-54, wherein the combination of polymorphisms is selected from Table 1, Table 4 or Table 27.
56. The system of any one of embodiments 52-55, wherein the combination of polymorphisms comprises a non-reference allele.
57. The system of embodiment 56, wherein the combination of polymorphisms is two polymorphisms.
58. The system of embodiment 56, wherein the combination of polymorphisms is three polymorphisms.
59. The system of any one of embodiments 52-58, wherein the subject has a disease comprising at least one of an inflammatory, a fibrostenotic, and a fibrotic, disease or condition.
60. The system of embodiment 59, wherein the disease is a TL1A mediated disease selected from the group consisting of inflammatory bowel disease (IBD), Crohn's disease (CD), obstructive CD, ulcerative colitis (UC), intestinal fibrosis, intestinal fibrostenosis, rheumatoid arthritis, and primary sclerosing cholangitis.
61. The system of any one of embodiments 52-60, wherein the sample consists of or comprises whole blood, plasma, serum, or tissue.
62. The system of any one of embodiments 52-61, wherein the combination of genotypes comprises a homozygous genotype at one or more polymorphism positions.
63. The system of any one of embodiments 52-62, where LD is defined by an r² value of at least 0.80, 0.85, 0.90, 0.95, or 1.0.
64. The system of any one of embodiments 52-63, wherein the combination of genotypes is associated with a risk that a subject has, or will develop, the positive therapeutic response to said inhibitor of TL1A activity or expression by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
65. The system of any one of embodiments 52-64, wherein said report comprises a recommendation for treatment of a disease of the subject.
66. The system of embodiment 65, wherein the treatment comprises administration of an inhibitor of TL1A activity or expression.
67. The system of embodiment 66, wherein the inhibitor of TL1A activity or expression comprises an antibody or antigen-binding fragment, peptide, or small molecule.
68. The system of embodiment 67, wherein the inhibitor of TL1A activity or expression comprises an anti-TL1A antibody.
69. The system of embodiment 68, wherein the anti-TL1A antibody comprises an amino acid sequence or a combination of amino acid sequences provided in Tables 16, 17, and 20.
70. The system of any one of embodiments 68-69, wherein the anti-TL1A antibody comprises an amino acid sequence or a combination of amino acid sequences provided in Section 5.4.1.
71. The system of any one of embodiments 68-69, wherein the anti-TL1A antibody binds to the same region of human TL1A as a reference antibody comprising an amino acid sequence or a combination of amino acid sequences provided in Tables 16, 17, and 20.
72. The system of any one of embodiments 68-69, wherein the anti-TL1A antibody binds to the same region of human TL1A as a reference antibody, the reference antibody comprising an amino acid sequence or a combination of amino acid sequences provided in Section 5.4.1.
73. The system of any one of embodiments 68-69, wherein the anti-TL1A antibody is a neutralizing TL1A antibody.
74. The system of any one of embodiments 68-69, wherein the anti-TL1A antibody is an antagonist of TL1A.
75. The system of any one of embodiments 52-74, wherein said combination of genotypes is determined with an assay comprising polymerase chain reaction (PCR), quantitative reverse-transcription PCR (qPCR), automated sequencing, genotype array, or a combination thereof.
76. The system of any one of embodiments 52-75, wherein said one or more computer processors is individually or collectively programed to generate said report that classifies said sample of said subject as a positive therapeutic response to said inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
77. The system of any one of embodiments 52-76, wherein said one or more computer processors is individually or collectively programed to generate said report that classifies said sample of said subject as a positive therapeutic response to said inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
78. The system of any one of embodiments 52-77, wherein said one or more computer processors is individually or collectively programed to generate said report that classifies said sample of said subject as a positive therapeutic response to said inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
79. The system of any one of embodiments 52-78, wherein said one or more computer processors is individually or collectively programed to generate said report that classifies said sample of said subject as a positive therapeutic response to said inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
80. The system of any one of embodiments 52-79, wherein the computer system comprises a central computing environment or a distributed computing environment.
81. Use of a composition comprising an inhibitor of TL1A for treating a subject, provided the subject is selected based on a combinations of genotypes at two or more polymorphism positions corresponding to a combination of polymorphisms provided in Table 1, Table 4, or Table 27, wherein the selection is based on PRI calculated from the combination of genotypes as follows:
   (1) if PRI has a positive correlation with MRS or RPS, then select the subject as having a positive therapeutic response to the inhibitor of TL1A activity or expression if PRI is above a cutoff; or
   (2) if PRI has a negative correlation with MRS or RPS, then select the subject as having a positive therapeutic response to the inhibitor of TL1A activity or expression if PRI is below a cutoff.
82. The use of embodiment 81, wherein the inhibitor of TL1A activity or expression is an anti-TL1A antibody.
83. The use of embodiment 82, wherein the anti-TL1A antibody is selected from Table 20.
84. The use of embodiment 82, wherein the anti-TL1A antibody comprises an amino acid sequence provided in Tables 16-17.
85. The use of embodiment 82, wherein the anti-TL1A antibody binds to the same region of human TL1A as a reference antibody selected from Table 20.
86. The use of embodiment 82, wherein the anti-TL1A antibody binds to the same region of human TL1A as a reference antibody, the reference antibody comprising an amino acid sequence provided in Tables 16-17.
87. The use of any one of embodiments 82-86, wherein the anti-TL1A antibody is a neutralizing TL1A antibody.
88. The use of any one of embodiments 82-86, wherein the anti-TL1A antibody is an antagonist of TL1A.
89. The use of any one of embodiments 82-88, wherein the genotype comprises at least two polymorphism positions corresponding to at least two polymorphisms provided in Table 1, Table 4, or Table 27.
90. The use of any one of embodiments 82-88, wherein the genotype comprises at least three polymorphism positions corresponding to at least three polymorphisms provided in Table 1, Table 4, or Table 27.
91. The use of any one of embodiments 82-88, wherein the genotype comprises at least four polymorphism positions corresponding to at least four polymorphisms provided in Table 1, Table 4, or Table 27.
92. The use of any one of embodiments 82-88, wherein the genotype comprises at least five polymorphism positions corresponding to at least four polymorphisms provided in Table 1, Table 4, or Table 27.
93. The use of any one of embodiments 82-88, wherein the genotype comprises at least six polymorphism positions corresponding to at least six polymorphisms provided in Table 1, Table 4, or Table 27.
94. The use of any one of embodiments 82-88, wherein the genotype comprises at least seven polymorphism positions corresponding to at least seven polymorphisms provided in Table 1, Table 4, or Table 27.
95. The use of any one of embodiments 82-88, wherein the genotype comprises at least eight polymorphism positions corresponding to at least eight polymorphisms provided in Table 1, Table 4, or Table 27.
96. The use of any one of embodiments 82-95, wherein the genotype comprises at least one polymorphism position corresponding to a non-reference allele.
97. The use of any one of embodiments 82-96, wherein the subjects selected based on the PRI calculated from the combination of genotypes have a positive therapeutic response to the inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
98. The use of any one of embodiments 82-97, wherein the subjects selected based on the PRI calculated from the combination of genotypes have a positive therapeutic response to the inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
99. The use of any one of embodiments 82-98, wherein the subjects selected based on the PRI calculated from the combination of genotypes have a positive therapeutic response to the inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
100. The use of any one of embodiments 82-99, wherein the subjects selected based on the PRI calculated from the combination of genotypes have a positive therapeutic response to the inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
101. A computer-implemented method for generating a report that classifies a sample, the method comprising:
   i) providing a computer system comprising:
      i. an input device configured to receive genotype data obtained from a DNA sample of a subject;
      ii. one or more computer processors individually or collectively programmed to:
         1. generate a PRI calculated based on a combination of genotypes determined from a DNA sample from a subject at polymorphism positions corresponding to a combination of polymorphisms provided in Table 1, Table 4 or Table 27, or a polymorphism in linkage disequilibrium (LD) therewith; and
         2. generate the report that classifies the sample of the subject based on the PRI; and
      iii. a user interface configured to display said report to a user.
   j) receiving, by the input device, the genotype data determined from the DNA sample of a subject;
   k) generating the PRI based upon the combination of genotypes; and
   l) generating the report that classifies the sample of the subject based on the PRI, wherein the classification is based on PRI calculated from the combination of genotypes as follows:
      (1) if PRI has a positive correlation with MRS or RPS, then classify said sample as from the subject having a positive therapeutic response to the inhibitor of TL1A activity or expression if PRI is above a cutoff; or
      (2) if PRI has a negative correlation with MRS or RPS, then classify said sample as from the subject having a positive therapeutic response to the inhibitor of TL1A activity or expression if PRI is below a cutoff.
102. The method of embodiment 101, wherein the combination of genotypes is based on the result of an assay of said DNA sample provided in a genotype data file that is analysed by the one or more computer processors.
103. The method of any one of embodiments 101-102, wherein the genotype data file comprises a plurality of genotypes, wherein each genotype of the plurality is at a polymorphism position corresponding to a polymorphism provided in Table 1, Table 4, or Table 27 represented as described in Table 28.
104. The method of any one of embodiments 102-103, wherein the assay of the DNA sample detects said combination of genotypes at said polymorphism provided in Table 1, Table 4, or Table 27.
105. The method of any one of embodiments 101-104, wherein the combination of genotypes comprises a non-reference allele at said polymorphism position corresponding to said polymorphism provided in Table 1, Table 4, or Table 27.
106. The method of any one of embodiments 101-104, wherein the combination of genotypes comprises polymorphism positions corresponding to at least two polymorphisms provided in Table 1, Table 4, or Table 27.
107. The method of any one of embodiments 101-104, wherein the combination of genotypes comprises polymorphism positions corresponding to at least three polymorphisms provided in Table 1, Table 4, or Table 27.
108. The method of any one of embodiments 101-107, wherein the subject has a disease comprising at least one of an inflammatory, a fibrostenotic, and a fibrotic, disease or condition.
109. The method of embodiment 108, wherein the disease is a TL1A mediated disease selected from the group consisting of inflammatory bowel disease (IBD), Crohn's disease (CD), obstructive CD, ulcerative colitis (UC), intestinal fibrosis, intestinal fibrostenosis, rheumatoid arthritis, and primary sclerosing cholangitis.
110. The method of any one of embodiments 101-109, wherein the sample consists of or comprises whole blood, plasma, serum, or tissue.
111. The method of any one of embodiments 101-110, wherein the combination of genotypes comprises a homozygous genotype at one or more polymorphism positions.
112. The method of any one of embodiments 101-111, where LD is defined by an r² value of at least 0.80, 0.85, 0.90, 0.95, or 1.0.
113. The method of any one of embodiments 101-112, wherein the combination of genotypes is associated with a risk that a subject has, or will develop, the positive therapeutic response to said inhibitor of TL1A activity or expression by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
114. The method of any one of embodiments 101-113, wherein said report comprises a recommendation for treatment of a disease of the subject.
115. The method of embodiment 114, wherein the treatment comprises administration of the inhibitor of TL1A activity or expression.
116. The method of any one of embodiments 101-115, wherein the inhibitor of TL1A activity or expression comprises an antibody or antigen-binding fragment, peptide, or small molecule.
117. The method of embodiment 116, wherein the inhibitor of TL1A activity or expression comprises an anti-TL1A antibody.
118. The method of embodiment 117, wherein the anti-TL1A antibody comprises an amino acid sequence or a combination of amino acid sequences provided in Tables 16-17 and 20.
119. The method of embodiment 117, wherein the anti-TL1A antibody comprises an amino acid sequence or a combination of amino acid sequences provided in Section 5.4.1.
120. The method of embodiment 117, wherein the anti-TL1A antibody binds to the same region of human TL1A as a reference antibody comprising an amino acid sequence or a combination of amino acid sequences provided in Tables 16-17 and 20.
121. The method of embodiment 117, wherein the anti-TL1A antibody binds to the same region of human TL1A as a reference antibody, the reference antibody comprising an amino acid sequence or a combination of amino acid sequences provided in Section 5.4.1.
122. The method of embodiment 117, wherein the anti-TL1A antibody is a neutralizing TL1A antibody.
123. The method of embodiment 117, wherein the anti-TL1A antibody is an antagonist of TL1A.
124. The method of any one of embodiments 101-123, wherein said combination of genotypes is determined with an assay comprising polymerase chain reaction (PCR), quantitative reverse-transcription PCR (qPCR), automated sequencing, genotype array, or a combination thereof.
125. The method of any one of embodiments 101-124, wherein said report classifies said sample of said subject as a positive therapeutic response to said inhibitor of TL1A activity or expression with a positive predictive value (PPV) of at least or about 25%, 26%, 27%, 28%, 29%, 30%, 31%, 32%, 33%, 34%, 35%, 40%, 45%, 50%, 51%, 55%, 60%, 65%, 70%, 75%, 80%, 85%, 90%, 91%, 92%, 93%, 94% 95%, 96%, 97%, 98%, 99%, or 100%.
126. The method of any one of embodiments 101-125, wherein said report classifies said sample of said subject as a positive therapeutic response to said inhibitor of TL1A activity or expression with a specificity of at least or about 50%, 55%, 60%, 65%, 70%, 75%, 80%, 85%, 90%, 91%, 92%, 93%, 94% 95%, 96%, 97%, 98%, 99%, or 100%.
127. The method of any one of embodiments 101-126, wherein said report classifies said sample of said subject as a positive therapeutic response to said inhibitor of TL1A activity or expression with a negative predictive value (NPV) of at least or about 50%, 55%, 60%, 65%, 70%, 75%, 80%, 85%, 90%, 91%, 92%, 93%, 94% 95%, 96%, 97%, 98%, 99%, or 100%.
128. The method of any one of embodiments 101-127, wherein said report classifies said sample of said subject as a positive therapeutic response to said inhibitor of TL1A activity or expression with a positive rate of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.
129. The method of any one of embodiments 101-128, wherein the computer system comprises a central computing environment or a distributed computing environment.
130. A computer-implemented system comprising at least one processor and instructions executable by the at least one processor to provide an application configured to determine a Response Probability Score (RPS) for a subject by performing operations comprising:
   (a) receiving genotype data obtained from a sample from the subject with an inflammatory, a fibrotic, or a fibrostenotic disease or condition, wherein the genotype data comprises a combination of polymorphisms;
   (b) applying a first statistical algorithm to the genotype data, the first statistical algorithm configured to produce a Model Risk Score (MRS) for the subject by performing operations comprising:
      (i) assigning a weighted numerical value to each polymorphism in the combination of polymorphisms to produce a plurality of weighted values; and
      (ii) summing the plurality of weighted values;
   (c) applying a second statistical algorithm to the MRS, the second statistical algorithm configured to perform a logarithmic function on the MRS to produce a Response Probability Score (RPS); and
   (d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition
131. A computer-implemented system comprising at least one processor and instructions executable by the at least one processor to provide an application configured to determine a Response Probability Score (RPS) for a subject by performing operations comprising:
   (a) receiving a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
   (b) receiving genotype data for a plurality of polymorphisms obtained from a sample from the subject;
   (c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
   (d) applying a logarithmic scale and a cutoff to the MRS to produce a Response Probability Score (RPS).
132. The computer-implemented system of any one of embodiments 130 to 131, wherein the RPS ranges from 0 to 1.
133. The computer-implemented system of any one of embodiments 130 to 132, wherein the cutoff is 0.5.
134. The computer-implemented system of embodiment 130, wherein the genotype data is a combination of single nucleotide polymorphisms (SNPs).
135. The computer-implemented system of any one of embodiments 130 to 134, wherein the RPS is calculated as 1/(1+e^{(-MRS)}), wherein the MRS is calculated as

$${β}_{0} + {\sum }_{i = 1}^{n} ( {β}_{i} ) \times ( {χ}_{i} ) ,$$

and wherein χᵢ is the mathematical representation of the ith single nucleotide polymorphisms (SNP) in the model and P, is the weight for the ith SNP in the model.
136. The computer-implemented system of any one of embodiments 130 to 135, wherein the MRS is calculated as

$${β}_{0} + {\sum }_{i = 1}^{n} ( {β}_{i} ) \times ( {χ}_{i} ) ,$$

and wherein χᵢ is the mathematical representation of the ith SNP in the model.
137. The computer-implemented system of any one of embodiments 131 to 136, wherein the SNP in the model is mathematically represented by χᵢ as:
   (i) 0 for homozygous reference alleles, 1 for heterozygous reference and alternative alleles, and 2 for homozygous alternative alleles;
   (ii) 1 for homozygous reference alleles, 1 for heterozygous reference and alternative alleles, and 0 for homozygous alternative alleles;
   (iii) 0 for homozygous reference alleles, 1 for heterozygous reference and alternative alleles, and 1 for homozygous alternative alleles;
   (iv) 0 for homozygous reference alleles, 0 for heterozygous reference and alternative alleles, and 1 for homozygous alternative alleles;
   (v) 1 for homozygous reference alleles, 0 for heterozygous reference and alternative alleles, and 0 for homozygous alternative alleles; and/or
   (vi) 0 for homozygous reference alleles, 1 for heterozygous reference and alternative alleles, and 0 for homozygous alternative alleles.
138. The computer-implemented system of any one of embodiments 131 to 137, wherein the combination of polymorphisms comprises one or more polymorphisms selected from Table 27, or a proxy polymorphism in linkage disequilibrium therewith as determined with an R² of at least 0.85, or a combination thereof.
139. The computer-implemented system of any one of embodiments 131 to 138, wherein the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.
140. The computer-implemented system of any one of embodiments 131 to 139, wherein the MRS or RPS is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.
141. The computer-implemented system of any one of embodiments 131 to 140, wherein
   (i) the MRS is calculated with a combination of polymorphisms selected from the combinations listed in column 2 of Table 31 and corresponding β coefficients listed in column 1 of Table 31; and/or
   (ii) the RPS is calculated with a combination of polymorphisms selected from the combinations listed in column 2 of Table 31 and corresponding β coefficients listed in column 1 of Table 31.
142. The computer-implemented system of any one of embodiments 131 to 141, wherein the combination of polymorphisms is detected in the sample by subjecting the sample to an assay configured to detect a presence of at least three nucleotides corresponding to nucleic acid position 501 within at least three of SEQ ID NOS: 2001-2048 and 2057-2059.

### 5.9 Sample Preparation and Genotyping Assays

As described further above, in various embodiments of the methods provided herein including in Sections 2, 5 (including but not limited to paragraphs of Section 5.2) and 7, the methods further comprise preparing the sample. In one embodiment, preparing sample comprises or consists of obtaining the sample from the subject (e.g. as described in Section 5.9.1). In another embodiments, preparing sample comprises or consists of releasing DNA from the sample (e.g. as described in Section 5.9.2). In a further embodiment, preparing sample comprises or consists of purifying the DNA (e.g. as described in Section 5.9.3). In yet another embodiments, preparing sample comprises or consists of amplifying the DNA (e.g. as described in Section 5.9.4). In one embodiment, preparing sample comprises or consists of obtaining the sample from the subject (e.g. as described in Section 5.9.1) and releasing DNA from the sample (e.g. as described in Section 5.9.2). In some embodiments, preparing sample comprises or consists of obtaining the sample from the subject (e.g. as described in Section 5.9.1) and purifying the DNA (e.g. as described in Section 5.9.3). In certain embodiments, preparing sample comprises or consists of obtaining the sample from the subject (e.g. as described in Section 5.9.1) and amplifying the DNA (e.g. as described in Section 5.9.4). In further embodiments, preparing sample comprises or consists of releasing DNA from the sample (e.g. as described in Section 5.9.2) and purifying the DNA (e.g. as described in Section 5.9.3). In one embodiment, preparing sample comprises or consists of releasing DNA from the sample (e.g. as described in Section 5.9.2) and amplifying the DNA (e.g. as described in Section 5.9.4). In other embodiments, preparing sample comprises or consists of purifying the DNA (e.g. as described in Section 5.9.3) and amplifying the DNA (e.g. as described in Section 5.9.4). In yet other embodiments, preparing sample comprises or consists of obtaining the sample from the subject (e.g. as described in Section 5.9.1), releasing DNA from the sample (e.g. as described in Section 5.9.2), and purifying the DNA (e.g. as described in Section 5.9.3). In some embodiments, preparing sample comprises or consists of obtaining the sample from the subject (e.g. as described in Section 5.9.1), releasing DNA from the sample (e.g. as described in Section 5.9.2) and amplifying the DNA (e.g. as described in Section 5.9.4). In certain embodiments, preparing sample comprises or consists of obtaining the sample from the subject (e.g. as described in Section 5.9.1), purifying the DNA (e.g. as described in Section 5.9.3) and amplifying the DNA (e.g. as described in Section 5.9.4). In some embodiments, preparing sample comprises or consists of releasing DNA from the sample (e.g. as described in Section 5.9.2), purifying the DNA (e.g. as described in Section 5.9.3) and amplifying the DNA (e.g. as described in Section 5.9.4). In other embodiments, preparing sample comprises or consists of obtaining the sample from the subject (e.g. as described in Section 5.9.1), releasing DNA from the sample (e.g. as described in Section 5.9.2), purifying the DNA (e.g. as described in Section 5.9.3), and amplifying the DNA (e.g. as described in Section 5.9.4).

Additionally, the disclosure provides various assays for determining or detecting the genotypes, combinations of genotypes, polymorphisms, or combinations of polymorphisms. As such, in various embodiments of the methods provided herein including in Sections 2, 5 (including but not limited to paragraphs of Section 5.2) and 7, determining or detecting the genotypes, combinations of genotypes, polymorphisms, or combinations of polymorphisms comprises or consists of assaying for the genotypes, combinations of genotypes, polymorphisms, or combinations of polymorphisms via any assays as described in Section 5.9.5. Alternatively, in various embodiments of the methods provided herein including in Sections 2, 5 (including but not limited to paragraphs of Section 5.2) and 7, the method further comprises assaying for the genotypes, combinations of genotypes, polymorphisms, or combinations of polymorphisms via any assays as described in Section 5.9.5.

### 5.9.1 Sample Collection from Patients

In some embodiments, the methods further comprise a step of obtaining the sample from the subject. Samples used for the genotyping, can be any samples collected from patients that contain the patient's DNA such as genomic DNA. In some specific embodiment of the methods provided herein, the sample is a bodily fluid sample. In one embodiment, the sample is a tissue sample. In one embodiment, the sample is a cell sample. In one embodiment, the sample is a blood sample. In one embodiment, the sample is a bone marrow sample. In one embodiment, the sample is a plasma sample. In one embodiment, the sample is a serum sample. In one embodiment, the sample is a saliva sample. In one embodiment, the sample is a cerebrospinal fluid sample.

### 5.9.2 DNA Release from Samples

DNA molecules can be released from the cells or tissues in patient's samples by various ways as known and practiced in the art. For example, the DNA molecules can be released by breaking up the host cells physically, mechanically, enzymatically, chemically, or by a combination of physical, mechanical, enzymatic and chemical actions. In some embodiments, the DNA molecules can be released from the samples by subjecting the samples to a solution of cell lysis reagents. Cell lysis reagents include detergents, such as triton, SDS, Tween, NP-40, and/or CHAPS. In other embodiments, the DNA molecules can be released from the samples by subjecting the samples to difference in osmolarity, for example, subjecting the samples to a hypotonic solution. In other embodiments, the DNA molecules can be released from the samples by subjecting the samples to a solution of high or low pH. In certain embodiments, the DNA molecules can be released from the samples by subjecting the samples to enzyme treatment, for example, treatment by lysozyme. In some further embodiments, the DNA molecules can be released from the samples by subjecting the samples to any combinations of detergent, osmolarity pressure, high or low pH, and/or enzymes (e.g. lysozyme).

Alternatively, the DNA molecules can be released from the host cells by exerting physical force on the host cells. In one embodiment, the DNA molecules can be released from the host cells by directly applying force to the host cells, e.g. by using the Waring blender and the Polytron. Waring blender uses high-speed rotating blades to break up the cells and the Polytron draws tissue into a long shaft containing rotating blades. In another embodiment, the DNA molecules can be released from the host cells by applying shear stress or shear force to the host cells. Various homogenizers can be used to force the host cells through a narrow space, thereby shearing the cell membranes. In some embodiments, the DNA molecules can be released from the host cells by liquid-based homogenization. In one specific embodiment, the DNA molecules can be released from the host cells by use a Dounce homogenizer. In another specific embodiment, the DNA molecules can be released from the host cells by use a Potter-Elvehjem homogenizer. In yet another specific embodiment, the DNA molecules can be released from the host cells by use a French press. Other physical forces to release the DNA molecules from host cells include manual grinding, e.g. with a mortar and pestle. In manual grinding, host cells are often frozen, e.g. in liquid nitrogen and then crushed using a mortar and pestle, during which process the tensile strength of the cellulose and other polysaccharides of the cell wall breaks up the host cells.

Additionally, the DNA molecules can be released from the samples by subjecting the samples to freeze and thaw cycles. In some embodiments, a suspension of samples are frozen and then thawed for a number of such freeze and thaw cycles. In some embodiments, the DNA molecules can be released from the samples by applying 1, 2, 3, 4, 5, 6, 7, 8, 9, 10, 11, 12, 13, 14, 15, 16, 17, 18, 19, or 20 freeze and thaw cycles to the samples.

The above described methods for releasing the DNA molecules from the samples are not mutually exclusive. Therefore, the disclosure provides that the DNA molecules can be released from the samples by any combinations of DNA releasing methods provide in this Section 5.9.2.

### 5.9.3 DNA Purification and/or Enrichment

In some embodiments, the methods provided herein further comprise purifying the subject's DNA molecules before genotyping assays. In one embodiment, the methods provided herein further comprise purifying the DNA by affinity purification. In one embodiment, the methods provided herein further comprise purifying the DNA by affinity purification with spin column. In one embodiment, the methods provided herein further comprise purifying the DNA by affinity purification with a positively charged matrix in the spin column that binds to the negatively charged DNA. In one embodiment, the methods provided herein further comprise purifying the DNA by affinity purification with a silica matrix in the spin column that binds to the DNA. In one embodiment, the methods provided herein further comprise purifying the DNA by affinity purification with an affinity tag that binds to the DNA or a fragment thereof. In some embodiments, the DNA bound to the affinity purification matrix can be eluted with an elution buffer or water, thereby yielding DNA with higher purity and higher concentration.

### 5.9.4 DNA Amplification

In some embodiments, the method provided herein comprises an DNA amplification step. The DNA amplification includes, for example, reactions comprising a forward and reverse primer, such that the primer extension products of the forward primer serve as templates for primer extension of the reverse primer, and vice versa. Amplification may be isothermal or non-isothermal. A variety of methods for amplification of target polynucleotides are available, and include without limitation, methods based on polymerase chain reaction (PCR). Conditions favorable to the amplification of target sequences by PCR can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some or all of which can be suitably altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization include adjustments to the type or amount of elements in the amplification reaction and/or to the conditions of a given step in the process, such as temperature at a particular step, duration of a particular step, and/or number of cycles. In some embodiments, an amplification reaction comprises at least or about 5, 10, 15, 20, 25, 30, 35, 40, 45, 50, or more cycles. In some embodiments, an amplification reaction comprises no more than 5, 10, 15, 20, 25, 35, 40, 45, 50, or more cycles. Cycles can contain any number of steps, such as 1, 2, 3, 4, 5, or more steps. Steps can comprise any temperature or gradient of temperatures, suitable for achieving the purpose of the given step, including but not limited to, 3′ end extension, primer annealing, primer extension, and strand denaturation. Steps can be of any duration, including but not limited to about or less than about 1, 5, 10, 15, 20, 25, 30, 35, 40, 45, 50, 55, 60, 70, 80, 90, 100, 120, 180, 240, 300, 360, 420, 480, 540, 600, or more seconds, including indefinitely until manually interrupted. In some embodiments, amplification is performed separately for each sample (e.g., for DNA purified from patient samples as described above). In some embodiments, amplification is performed separately for each sample (e.g., for DNA purified from patient samples as described above), but together on one PCR plate (e.g. 96 well plate wherein up to 96 PCR reactions were performed together). In some embodiments, amplification is performed before or after pooling of target polynucleotides (e.g., DNA purified from patient samples as described above) from independent samples or aliquots. Non-limiting examples of PCR amplification techniques include quantitative PCR (qPCR or real-time PCR), digital PCR, and target-specific PCR.

Non-limiting examples of polymerase enzymes for use in PCR include thermostable DNA polymerases, such as Thermus thermophilus HB8 polymerase; Thermus oshimai polymerase; Thermus scotoductus polymerase; Thermus thermophilus polymerase; Thermus aquaticus polymerase (e.g., AmpliTaq® FS or Taq (G46D; F667Y); Pyrococcus furiosus polymerase; Thermococcus sp. (strain 9° N-7) polymerase; Tsp polymerase; Phusion High-Fidelity DNA Polymerase (ThermoFisher); and mutants, variants, or derivatives thereof. Further examples of polymerase enzymes useful for some PCR reactions include, but are not limited to, DNA polymerase I, mutant DNA polymerase I, Klenow fragment, Klenow fragment (3′ to 5′ exonuclease minus), T4 DNA polymerase, mutant T4 DNA polymerase, T7 DNA polymerase, mutant T7 DNA polymerase, phi29 DNA polymerase, and mutant phi29 DNA polymerase. In some embodiments, a hot start polymerase is used. A hot start polymerase is a modified form of a DNA Polymerase that requires thermal activation. Typically, the hot start enzyme is provided in an inactive state. Upon thermal activation the modification or modifier is released, generating active enzyme. A number of hot start polymerases are available from various commercial sources, such as Applied Biosystems; Bio-Rad; ThermoFisher, New England Biolabs; Promega; QIAGEN; Roche Applied Science; Sigma-Aldrich; and the like.

In some embodiments, primer extension and amplification reactions comprise isothermal reactions. Non-limiting examples of isothermal amplification technologies are ligase chain reaction (LCR) (see e.g., U.S. Pat. Nos. 5,494,810 and 5,830,711); transcription mediated amplification (TMA) (see e.g., U.S. Pat. Nos. 5,399,491, 5,888,779, 5,705,365, 5,710,029); nucleic acid sequence-based amplification (NASBA) (see e.g., U.S. Pat. No. 5,130,238); signal mediated amplification of RNA technology (SMART) (see e.g., Wharam et al., Nucleic Acids Res. 2001, 29, e54); strand displacement amplification (SDA) (see e.g., U.S. Pat. No. 5,455,166); thermophilic SDA (see e.g., U.S. Pat. No. 5,648,211); rolling circle amplification (RCA) (see e.g., U.S. Pat. No. 5,854,033); loop-mediated isothermal amplification of DNA (LAMP) (see e.g., U.S. Pat. No. 6,410,278); helicase-dependent amplification (HDA) (see e.g., U.S. Pat. Appl. 20040058378); exponential amplification methods based on SPIA (see e.g., U.S. Pat. No. 7,094,536); and circular helicase-dependent amplification (cHDA) (e.g., U.S. Pat. Appl. 20100075384).

### 5.9.5 Determination of Genotypes

Genotypes can be determined by hybridization of probes to the amplified DNA (e.g. as described above), wherein the probes are specific for each polymorphism (e.g. each SNP) and a short sequence flanking the polymorphism. Alternatively, genotypes can be determined by adding probes to the PCR reaction mixture and having the probe hybridize with the PCR product during each cycle of the PCR amplification.

In one embodiment, genotypes (e.g. SNPs) can be determined by adding a fluorogenic probe, complementary to the target sequence (e.g. the short sequence encompassing the polymorphisms), to the PCR reaction mixture. This probe is an oligonucleotide with a reporter dye attached to the 5′ end and a quencher dye attached to the 3′ end such that the reporter and the quencher are in close proximity in the probe in a default configuration (e.g. with a short hairpin structure or due to the short length of the probe). When the probe is not bound to the target or hydrolyzed by the polymerase, the quencher and the fluorophore remain in proximity to each other, separated only by the length of the probe, leaving only a background fluorescence. During PCR, the probe anneals specifically between the forward and reverse primer to the internal region of the PCR product encompassing the polymorphism. The polymerase then carries out the extension of the primer and replicates the template to which the probe is bound. The 5′ exonuclease activity of the polymerase cleaves the probe, releasing the reporter molecule away from the close vicinity of the quencher. The fluorescence intensity of the reporter dye increases as a result. This process repeats in every cycle and does not interfere with the accumulation of PCR product, resulting in continuous increase of the reporter fluorescence intensity. The genotypes (e.g. polymorphisms and SNPs) are determined by the fluorescence signal. The probes for the genotypes (e.g. polymorphisms and SNPs) are often 10-30 bases in length and designed to discriminate between its target and a highly related mismatch sequence. For this discrimination to be successful, the probes are designed to provide a difference in the melting temperatures of the duplex with the intended target and the duplex with highly related mismatch sequence (i.e., a high ΔTm value). The length and sequence of the probe is designed, at least in part, to optimize such ΔTm. In some embodiments, the probes are DNA molecules. In some embodiments, the probes are RNA molecules. In some embodiments, the probes are locked nucleic acids (LNA). The LNA probes provide significant differences in ΔTm, often around 20° C. for single mismatches, due to the high specificity and high affinity of the LNA probes. In some embodiments, the reporter dye is a fluorescence dye.

In some embodiments, the genotyping can be performed in a multiplexing assay. A multiplexing assay refers to an assay that can detect or determine multiple genotypes, e.g. multiple polymorphisms or multiple SNPs in the sample. Multiplexing can be achieved via physical separation or multiplication of the same sample, e.g. running a 96-well plate PCR with specific PCR primer and SNP detecting probe per well, but multiple SNP detecting probes for the sample per plate, thereby detecting multiple genotypes for a sample in one 96-well PCR. Multiplexing can also be achieved by running a PCR reaction with multiple PCR primers and multiple SNP detecting probes, with each probe attached to a fluorescent dye of a unique color, thereby distinguishing the SNPs in the single reaction via unique fluorescence signal associated with each SNP. In one embodiment, the methods provide herein comprise a multiplexing PCR. In another embodiment, the methods provided herein comprise a multiplexing PCR with each genotype (e.g. each polymorphism or SNP) detected in a different fluorescence signal. Other multiplexing PCR methods, such as multiplexed qPCR or multiplexed digital PCR can be used here as well. In one embodiment, the methods provided herein comprise multiplexed qPCR. In another embodiment, the methods provided herein comprise multiplexed digital PCR.

Similarly, other hybridization or PCT based can also be used to detect or determining the genotypes (e.g. polymorphisms or SNPs) and are provided herein. For example, in some embodiments, the genotypes (e.g. polymorphisms or SNPs) are detected or determined via dynamic allele-specific hybridization such as described in Genome Res. 2001 January; 11(1): 152-162, molecular beacons such as described in Clin Chem Lab Med. 2003 April; 41(4):468-74, SNP microarrays as commercially available from Affymetrix.

Alternatively, the genotype (e.g. the polymorphisms or SNPs) can be detected or determined by sequencing the DNA purified from the sample as described in Section 5.9.3 or the amplified DNA described in Section 5.9.4. In some embodiments, the methods comprise sequencing the purified DNA or the amplified DNA. In some embodiments, the methods comprise sequencing products of the amplification with a primer different from the primers used in the amplification. In some embodiments, the methods comprise sequencing the purified DNA or the amplified DNA by next generation sequencing (NGS).

A variety of sequencing methodologies are available, particularly high-throughput sequencing methodologies. Examples include, without limitation, sequencing systems manufactured by Illumina (ILLUMINA next generation sequencing, sequencing systems such as HiSeq® and MiSeq®), Life Technologies (Ion Torrent®, SOLiD®, etc.), Roche's 454 Life Sciences systems, Pacific Biosciences systems, nanopore sequencing platforms by Oxford Nanopore Technologies, etc, which manufactures public protocols and instructions for sequencing are each hereby incorporated in their entirety by reference. In some embodiments, sequencing comprises producing reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate, an example description of which can be found in U.S. Pat. No. 6,210,891. According to some sequencing methodologies, the primer/template/polymerase complex is immobilized upon a substrate and the complex is contacted with labeled nucleotides. Further non-limiting examples of sequencing technologies are described in US20160304954, U.S. Pat. Nos. 7,033,764, 7,416,844, and WO2016077602. In some embodiments, the methods comprise sequencing the purified DNA or the amplified DNA by next generation sequencing (NGS)

In some cases, sequencing reactions of various types, as described herein, may comprise a variety of sample processing units. Sample processing units may include but are not limited to multiple lanes, multiple channels, multiple wells, and other mean of processing multiple sample sets substantially simultaneously. Additionally, the sample processing unit may include multiple sample chambers to facilitate processing of multiple runs simultaneously. In some embodiments, simultaneous sequencing reactions are performed using multiplex sequencing. In some embodiments, polynucleotides are sequenced to produce about or more than about 5000, 10000, 50000, 100000, 1000000, 5000000, 10000000, or more sequencing reads in parallel, such as in a single reaction or reaction vessel. Subsequent data analysis can be performed on all or part of the sequencing reactions. Where polynucleotides are associated with an index sequence, data analysis can comprise grouping sequences based on index sequence for analysis together, and/or comparison to sequences associated with one or more different indices.

In some embodiments, sequence analysis comprises comparison of one or more reads to a reference sequence (e.g., a control sequence, sequencing data for a reference population, or a reference genome), such as by performing an alignment. In a typical alignment, a base in a sequencing read alongside a non-matching base in the reference indicates a polymorphism (e.g. SNP) at that nucleoposition. Similarly, where one sequence includes a gap alongside a base in the other sequence, an insertion or deletion mutation (an “indel”) is inferred to have occurred. When it is desired to specify that one sequence is being aligned to one other, the alignment is sometimes called a pairwise alignment. Multiple sequence alignment generally refers to the alignment of two or more sequences, including, for example, by a series of pairwise alignments. Examples of algorithms for performing alignments include, without limitation, the Smith-Waterman (SW) algorithm, the Needleman-Wunsch (NW) algorithm, algorithms based on the Burrows-Wheeler Transform (BWT), and hash function aligners such as Novoalign (Novocraft Technologies; available at www.novocraft.com), ELAND (Illumina, San Diego, Calif.), SOAP (available at soap.genomics.org.cn), and Maq (available at maq.sourceforge.net). One exemplary alignment program, which implements a BWT approach, is Burrows-Wheeler Aligner (BWA) available from the SourceForge web site maintained by Geeknet (Fairfax, Va.). An alignment program that implements a version of the Smith-Waterman algorithm is MUMmer, available from the SourceForge web site maintained by Geeknet (Fairfax, Va.). Other non-limiting examples of alignment programs include: BLAT from Kent Informatics (Santa Cruz, Calif.); SOAP2, from Beijing Genomics Institute (Beijing, Conn.) or BGI Americas Corporation (Cambridge, Mass.); Bowtie; Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator from Real Time Genomics, Inc. (San Francisco, Calif.); Novoalign from Novocraft (Selangor, Malaysia); Exonerate, European Bioinformatics Institute (Hinxton, UK), Clustal Omega, from University College Dublin (Dublin, Ireland); and ClustalW or ClustalX from University College Dublin (Dublin, Ireland).

Furthermore, barcode IDs can be introduced to the amplified DNA for each sample and for each SNP via the PCR primer pairs for the PCR reaction. “Barcode ID,” “barcode,” or “ID,” refers to a sequence or a series of sequences that can be used to identify, directly or indirectly through the identification information contained in the sequence or the series of the sequences. Such an ID can be a nucleic acid molecule with a given sequence, a unique fluorescent label, a unique colorimetric label, a sequence of the fluorescent labels, a sequence of the colorimetric label, or any other molecules or combination of molecules, so long as molecules or the combination of molecules used as IDs can identify or otherwise distinguish a particular target or sample from other targets or samples and be correlated with the intended target or sample. Nucleic acid molecules used as such IDs are also known as barcode sequences. Such an ID can also be a further derivative molecule that contains the information derived from but is non-identical to the original ID, so long as such derived molecules or the derived information can identify or otherwise distinguish a particular target or sample from other targets or samples and be correlated with the intended target or sample. For example, a nucleic acid ID can include both the original nucleic acid barcode sequence and/or the reverse complement of the original nucleic acid barcode sequence, as both can distinguish and be correlated with the intended target or sample. The barcode sequence can be any sequences, natural or non-natural, that are not present without being introduced as barcode sequences in the intended sample, the intended target, or any part of the intended sample or target, so that the barcode sequence can identify and be correlated with the sample or target. A barcode sequence can be unique to a single nucleic acid species in a population or a barcode sequence can be shared by several different nucleic acid species in a population. Each nucleic acid probe in a population can include different barcode sequences from all other nucleic acid probes in the population. Alternatively, each nucleic acid probe in a population can include different barcode sequences from some or most other nucleic acid probes in a population. For a specific example, all the amplified DNA generated from one patient sample can have the same sample barcode sequence (sample ID). For another example, all the amplified DNA generated for a target SNP can have a unique target barcode sequences (“target IDs”). Therefore, the disclosure provides that each patient sample can be identified by the patient ID and the PCR product for each SNP can be identified by a target ID, thereby providing multiplexing for multiple samples and multiple SNP detection in one reaction.

As such, in one embodiment, the methods comprising detecting multiple SNPs in a multiplexing assay by incorporating a unique target ID to each PCR primer pairs used to amplify the sequence fragment containing each SNP. In one embodiment, the methods comprising detecting multiple SNPs in a multiplexing assay by incorporating a unique sample ID to all PCR primer pairs used to amplify one patient sample. In another embodiment, the methods comprising detecting multiple SNPs in a multiplexing assay by (1) incorporating a unique target ID to each PCR primer pairs used to amplify the sequence fragment containing each SNP and (2) incorporating a unique sample ID to all PCR primer pairs used to amplify one patient sample.

The amplified DNA in the multiplexing assay methods disclosed herein can be detected by multiplexed qPCR, multiplexed digital PCR, or NGS. For example, in some embodiments, the amplified DNA in the multiplexing assay methods disclosed herein can be detected by NGS. The use of NGS to detect the amplified DNA generated by assay methods disclosed herein include some advantages. For example, by incorporating target and sample ID tags into the amplified DNA, as described herein, NGS is capable of multiplexed detection at a very large scale. For example, NGS can read a pool of 100 samples, each comprising 10 targets (i.e. 1000-plex) in a single run. This significantly reduces the per data point cost. Additionally, NGS can count and aggregate the number of molecules of the same sequence, providing digital quantification at single molecule resolution. Furthermore, a wide range of error correction algorithms, such as parity check, Hamming codes (e.g. Bystrykh, PLoS ONE 7(5): e36852 (2012)), and Levenshtein codes (e.g. Buschmann, BMC Bioinformatics. 2013; 14: 272 (2013)) can be used from communication theory and applied herein to reduce false counts so that NGS based quantification can achieve high precision without repeated sequencing.

As such, provided herein are also assay methods comprising simultaneously detecting at least two SNPs in a patient sample, by simultaneously detecting the unique target IDs associated with each SNP. Also provided herein are assay methods comprising simultaneously detecting at least two SNPs in at least two samples, by simultaneously detecting the unique target IDs associated with each SNP and the unique sample IDs associated with each sample.

In some embodiments, the assay methods provided herein simultaneously detect at least two, at least three, at least four, at least five, at least six, at least seven, at least eight, at least nine, at least ten, or more SNPs in at least two, at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the amplified DNA with each sample. In some embodiments, the assay methods provided herein simultaneously detect at least two, at least three, at least four, at least five, at least six, at least seven, at least eight, at least nine, at least ten, or more SNPs in a sample by detecting unique target IDs in the amplified DNA with each sample. In some embodiments, the assay methods provided herein simultaneously detect about two, about three, about four, about five, about six, about seven, about eight, about nine, about ten, or more SNPs in about two, about three, about four, about five, about six, about seven, about eight, about nine, about ten, about 15, about 20, about 30, about 40, about 50, about 60, about 70, about 80, about 90, about 100, about 150, about 200, about 250, about 300, about 350, about 400, about 450, or about 500 samples by simultaneously detecting unique sample IDs and unique target IDs in the amplified DNA with each sample. In some embodiments, the assay methods provided herein simultaneously detect about two, about three, about four, about five, about six, about seven, about eight, about nine, about ten, or more SNPs in a sample by detecting unique target IDs in the amplified DNA with each sample.

In certain embodiments, the assay methods provided herein simultaneously detect at least two, at least three, at least four, at least five, at least six, at least seven, at least eight, at least nine, at least ten, or more SNPs in a sample by detecting unique fluorescence signal associated with each SNP. In some embodiments, the assay methods provided herein simultaneously detect about two, about three, about four, about five, about six, about seven, about eight, about nine, about ten, or more SNPs in a sample by detecting unique fluorescence signal associated with each SNP.

### 6. Other Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “and/or” as used in a phrase with a list of members is intended to include all members individually and all combination of full or partial list of members. For example, a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “model,” when used in the context of CDx model or SNP model, or other similar contexts, refer to a system, method, process, and/or composition of (i) a combination of SNPs used in a patient selection criteria, (ii) the description, the identity, or the representations (e.g. mathematical or logical representation) of the SNPs used in the patient selection criteria, and/or (iii) the methods, processes, rules, systems, and/or mathematical operations used to determine the readout of the patient selection criteria based on (i) and (ii), wherein the patient selection criteria is for selecting patients suitable for treatment with an inhibitor of TL1A activity or expression.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

In some embodiments, the terms “individual” or “subject” are used interchangeably and refer to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition. In some embodiments, the subject is a “patient,” that has been diagnosed with a disease or condition described herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of polynucleotide sequences within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNV is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the dbSNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, which tend to be inherited together in a reference population. In some embodiments, a haplotype comprises particular polymorphism or another polymorphism in linkage disequilibrium (LD) therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r² value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteroids, anti-TNF therapy, anti-a4-b7 therapy (vedolizumab), anti-IL12p40 therapy (ustekinumab), Thalidomide, and Cytoxin.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician. In some cases, therapeutically effective amount of the drug reduces the severity of symptoms of the disease or disorder. In some instances, the disease or disorder comprises inflammatory bowel disease (IBD), Crohn's disease (CD), or ulcerative colitis (UC). In some instances, the IBD, CD, and/or UC are severe or medically refractory forms of the IBD, CD, and/or UC. Non-limiting examples of symptoms of IBD, CD, and/or UC include, but are not limited to, diarrhea, fever, fatigue, abdominal pain, abdominal cramping, inflammation, ulceration, nausea, vomiting, bleeding, blood in stool, reduced appetite, and weight loss.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-12 antibody, and anti-Cbir1 flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### 7. EXAMPLES

The following examples are included for illustrative purposes only and are not intended to limit the scope of the invention.

### 7.1 Example 1: Overview of the Identification of Genotypes

Tumor Necrosis Factor (Ligand) Superfamily, Member 15 (TNFSF15) has been determined to be significantly associated with inflammatory bowel disease (IBD), including Crohn's disease (CD), by Genome Wide Association Studies (GWAS) (e.g., cases versus controls). In addition, increased levels of TL1A (e.g., RNA and protein) are associated with IBD, including CD. Therefore, therapeutic strategies targeting TNFSF15 (TL1A) expression or activity offer a promising approach for the treatment of IBD. Disclosed herein is the identification of polymorphisms that are associated with, and therefore predictive of, an increase in TNFSF15 (TL1A) expression in patients with IBD, including CD, using a machine learning approach.

A polygenic risk score (PRS) adapted to identify individuals at risk for having increased TNFSF15 (TL1A) was applied to a cohort of CD patients recruited at the Cedars-Sinai Medical Center. A machine learning algorithm (e.g., XGBoost) was used to identify combinations of polymorphisms associated with increased TNFSF15 (TL1A) expression or activity. The resulting 41 polymorphisms, and a possible combinations of polymorphisms, have optimal prediction precision across multiple iterations of training the machine learning algorithm, which was able to analyze large combinations of polymorphism interactions (e.g., including non-linear interactions) in an efficient manner, which traditional GWAS methodologies cannot achieve. The resulting polymorphisms are useful for selecting a subject, who may or may not be diagnosed with IBD, who may exhibit a therapeutic response to an TNFSF15 (TL1A)-targeting therapeutic agent (e.g., neutralizing anti-TL1A antibody).

### 7.2 Example 2: Calculation of TNFSF15 PRS

A polygenic risk score (PRS) based on polymorphisms within multiple genes of interest (e.g., involved in the TL1A-mediated inflammatory pathways) and their associated weights in each respective reference population was calculated. The PRS is referred to herein as the “TNFSF15 PRS.” The polymorphisms were selected from multiple GWAS based on a defined distances from the transcription start and stop sites for the gene(s) of interest (e.g., 250 kilobases upstream and downstream). Each GWAS was to define the individual weights for contribution of a polymorphism to the total score. The GWAS used include, but are not limited to, Jostins et al., 2012. Nature. 491:119-124, Liu et al., 2015. Nat Genet. 47:979-986, Ellinghaus et al., 2016. Nat Genet. 48:510-518, Huang et al., 2017. Nat Genet. 49:256-261, and de Lange et al., 2017. Nat Genet. 48:256-261.

To confirm the relevance of inclusion of a polymorphism within the TNFSF15 PRS, the polymorphisms were cross-checked by (i) evidence of cis-QTL, where the SNP is directly associated with target gene expression in tissues and (ii) sensitivity analysis, where selected polymorphisms are removed from the TNFSF15 PRS and a regression analysis is run against disease susceptibility and subclinical phenotypes, thus highlighting relevant polymorphisms to disease risk. In some cases, the polymorphisms were subjected to sensitivity analysis, and in other cases, they were not. For example, in some cases, only those polymorphisms with questionable association to the pathway TNFSF15 PRS (e.g., no eQTL or multiple genes within the loci) are subjected to sensitivity analysis.

Patients with Crohn's disease (CD) were recruited. The diagnosis of each patient was based on standard endoscopic, histologic, and radiographic features. Blood samples were collected from patients at the time of enrollment. Genotyping was performed using Immunochip (ICHIP) per manufacturer's protocol on all samples collected.

A TNFSF15 PRS was calculated for Caucasian patients within a Cedars-Sinai CD cohort from Example 1, based on the defined set of polymorphisms selected in this Example 2, which are provided in Table 4. An exemplary calculation of TNFSF15 PRS is outlined in Li et al., 2018. Inflamm Bowel Dis. 12; 24(11):2413-2422. The TNFSF15 PRS is calculated as the weighted sum of the number of risk alleles carried by each patient (in the Cedars-Sinai CD cohort) (0, 1, or 2) at each loci for the genes described in this Example 2, divided by a total number of genetic variants used in the model. The same calculations were performed for each individual belonging to a reference group, thereby generating a range of raw scores (observed range). The resulting TNFSF15 PRS is generated by comparing the score of each patient with the observed range observed in the reference group.

TABLE 4
TNFSF15 Polygenic Risk Score (PRS) Polymorphisms

| | | | Minor | | | | |
|---|---|---|---|---|---|---|---|
| | | Minor | Allele | Major | | | Seq |
| rsID | Illumina_id | Allele | Frequency | Allele | Chromosome | Gene | ID No. |
| rs11221332 | imm_11_127886184 | A | 0.232475 | G | chr11 | ETS1 | 41 |
| rs7134599 | imm_12_66786342 | A | 0.381954 | G | chr12 | IFNG | 42 |
| rs6062496 | imm_20_61799543 | G | 0.406514 | A | chr20 | TNFRSF6B | 43 |
| rs4246905 | imm_9_116593070 | A | 0.270925 | G | chr9 | TNFSF15 | 8 |
| rs7468800 | imm_9_116631826 | A | 0.124622 | C | chr9 | TNFSF15; TNFSF8 | 44 |
| rs1569328 | rs1569328 | A | 0.14869 | G | chr14 | U2; FOS | 45 |
| rs2284553 | rs2284553 | A | 0.386244 | G | chr21 | IFNGR2 | 46 |
| rs6062504 | rs6062504 | A | 0.27128 | G | chr20 | ZGPAT | 47 |
| rs7556897 | rs7556897 | G | 0.334936 | A | chr2 | SLC19A3; CCL20 | 48 |

### 7.3 Example 3: XGBoost Machine Learning Algorithm Trained on Binary Classifiers Based on TNFSF15 Polygenic Risk Score

A binary classifier to be used in the XGBoost machine learning platform for CD samples was created based on the distribution of the TNFSF15 PRS scores (calculated in Example 2) across the CD cohort. In this example, patient samples from the CD cohort were classified as 0 if their TNFSF15 PRS was ≤25th percentile of the TNFSF15 PRS CD distribution. Patient samples were classified as 1 if their TNFSF15 PRS was ≥75th percentile of the TNFSF15 PRS CD distribution.

Once the initial classifier of TNFSF15 SNPs was established, the XGBoost algorithm was optimized for the polymorphisms and implemented to generate an initial list of candidate polymorphisms. XGBoost is rooted in the gradient boosted decision trees, which in contrast to lasso and ridge regression methods, incorporates complex non-linear feature interactions into prediction models in a non-additive form. Exemplary optimization and implementation procedures are provided in Behravan et al. Sci Rep. 2018; 8:13149. A total of ten iterations of 5-fold cross validation were used to obtain an initial list of candidate polymorphisms. These polymorphisms were further filtered/optimized using an adaptive iterative search procedure as outlined in Behravan et al. as well as support vector machines (SVM) resulting in a final list of SNPs, which had high prediction precision (>90%) for the TNFSF15 PRS binary classifier.

### 7.4 Example 4: XGBoost Machine Learning Algorithm Trained on Binary Classifiers Based on TNFSF15 Protein Expression

Patients with Crohn's disease (CD) were recruited. The diagnosis of each patient was based on standard endoscopic, histologic, and radiographic features. Blood samples were collected from the patients. All patients were genotyped either by Illumina ImmunoArray or polymerase chain reaction (PCR) under standard hybridization conditions. Peripheral blood mononuclear cells (PBMCs) were isolated from the blood samples. The PMBCs were stimulated in vitro with immune complex. Supernatants were collected from unstimulated samples and from stimulated samples at 6, 18, 24, and 72 hours. Soluble TL1A protein in the supernatants was quantified using a plate-based ELISA using and monoclonal antibodies at all time points.

Binary classifiers to be used in the XGBoost machine learning platform for the samples were derived using TL1A protein expression levels at 6 hours. The classifier at 6 hours reflects absolute levels of TL1A protein expression at that time point. Additional binary classifiers were derived using the results of clustering of samples (k=2 and k=3 groups) based on TNFSF15 protein expression across 6, 18, 24, and 72-hour time points. The classifiers at the combination of time points (e.g., 6, 18, 24, and 72) reflect a rate of production of TL1A between time points. The clustering was performed using the TMixClust Bioconductor package as described in Golumbeanu et al. (“Clustering time series gene expression data with TMixClust 2018). A set of predictive SNPs was obtained from each of the three XGBoost analyses of the three classifiers. The polymorphisms identified here were compared to the list of polymorphisms generated in Example 3 to identify only those polymorphisms that overlap between the two analyses.

Determination of overlaps of SNPs was performed on the gene annotation (refGene annotation) of the generated SNPs and not on the actual ICHIP or dbSNP reference sequence identification numbers. Only polymorphisms with minor allele frequencies (MAF)≥0.1 and the beta coefficients (from support vector machine (SVM) runs) with absolute values≥0.1 were kept. This resulted in 129 polymorphisms remaining for further analysis. The 9 polymorphisms used in the TNFF15 PRS (Table 4) were also added to this list of SNPs, resulting in a total of 138 SNPs.

### 7.5 Example 5: Implementation of Market Basket Analysis to Determine Non-Linear Polymorphism Combination Rules Associated with CD

In order to further filter out SNPs not strongly associated with clinical phenotypes, a market basket analysis approach was used to determine combination rules for the polymorphisms associated with Crohn's Disease (CD) clinical phenotypes. An exemplary market basket analysis is described in Breuer et al. Int J Bipolar Disord. 2018; 6:24). The initial dataset on CD localization and CD characterization was obtained from 1,803 CD cohorts from Cedars-Sinai Medical Center. The RUDI (Rule Discoverer) program (dominant minor model), also described in Breuer et al., was run on the 1,803 CD cohorts using the genotypes of the previously generated 138 SNPs (Example 4). The analysis resulted in 57 rules with significant associations with clinical phenotypes for Crohn's Disease. The clinical phenotypes used in the analysis were CD location (ileum, colon, and ilealcolon), CD characterization (non-stricturing/non-penetrating, stricturing, stricturing and internal penetrating, and isolated internal penetrating), and presence of perianal disease. The 57 association rules consisted of only 89 out of the 138 input polymorphisms from Example 4. Therefore, only these 89 polymorphisms were considered for further evaluation.

Finally, support vector machines (SVM) analysis based on the 3 binary classifiers described in Example 3 and Example 4 were re-applied to this list of 89 polymorphisms. Only XGBoost models (and their corresponding polymorphisms) with a prediction precision≥0.70 were maintained. And further, only the polymorphisms from these models with an SVM coefficient≥0.25 or an SVM coefficient≤−0.25 were kept. Applying these filters, a total of 41 polymorphisms were generated when analyzing all 3 classifiers. These polymorphisms and reference alleles are provided in Table 1. The nucleic acid sequences comprising the polymorphisms are provided in SEQ ID NOS: 1-41, or 57-59, and the position of the polymorphism within the nucleic acid sequence is indicated with a non-nucleobase letter (e.g., V, R, S, and the like). The sequences provided are from build 151.

The polymorphisms identified in the analysis provided in the Examples above may be used to predict a positive therapeutic response in a subject or a patient to an inhibitor of TL1A activity or expression (e.g., anti-TL1A antibody), either alone, or in combinations (e.g., 2, 3, 4, 5, 6, 7, and so forth). The polymorphisms described herein may be used in a diagnostic or prognostic test to identify a subject suitable for treatment with an inhibitor of TL1A activity or expression to treat a disease or condition described herein in the subject. In some cases, the diagnostic is a companion diagnostic test, such as for example, a TL1A companion diagnostic test (“TL1A CDx”).

In a non-limiting example, any combination of three polymorphisms, each selected from Table 1, may be used to predict a positive therapeutic response to an inhibitor of TL1A activity or expression. Exemplary three-polymorphism combinations include: imm_9_116608587, imm_11_127948309, and rs1892231; imm_9_116608587, imm_11_127948309, and rs9806914; imm_9_116608587, imm_11_127948309, and imm_21_44478192; imm_9_116608587, imm_11_127948309, and imm_21_44479552 imm_9_116608587, rs1892231, and rs9806914; imm_9_116608587, rs1892231, and imm_21_44478192; imm_9_116608587, rs1892231, and imm_21_44479552; imm_9_116608587, rs9806914, and imm_21_44478192; imm_9_116608587, rs9806914, and imm_21_44479552; imm_9_116608587, imm_21_44478192, and imm_21_44479552; imm_11_127948309, rs1892231, and rs9806914; imm_11_127948309, rs1892231, and imm_21_44478192; imm_11_127948309, rs1892231, and imm_21_44479552; imm_11_127948309, rs9806914, and imm_21_44478192; imm_11_127948309, rs9806914, and imm_21_44479552; imm_11_127948309, imm_21_44478192, and imm_21_44479552; rs1892231, rs9806914, and imm_21_44478192; rs1892231, rs9806914, and imm_21_44479552; rs1892231, imm_21_44478192, and imm_21_44479552; and rs9806914, imm_21_44478192, and imm_21_44479552.

Table 5 provides a table with the position of each polymorphism provided in Table 1 within the human genome according to GRCh38.p13 Primary Assembly. The nucleic acid sequence flanking each polymorphism is identified with the relevant SEQ ID NO.

TABLE 5
GRCh38.p13 Primary Assembly Positions of Polymorphisms

| | SEQ | |
|---|---|---|
| | ID | |
| dbSNP | NO: | SNP_SEQ_GRCh38.p13 Primary Assembly |
| rs11897732 | 2060 | >NC_000002.12: 43313747-43314246 |
| | | Homo sapiens chromosome 2 |
| | | SEQ = [G/A] |
| | | >NC_000002.12: 43314248-43314747 |
| | | Homo sapiens chromosome 2 |
| rs6740739 | 2061 | >NC_000002.12: 43628004-43628503 |
| | | Homo sapiens chromosome 2 |
| | | SEQ = [G/A] |
| | | >NC_000002.12: 43628505-43629004 |
| | | Homo sapiens chromosome 2 |
| rs17796285 | 2062 | >NC_000008.11: 11266446-11266945 |
| | | Homo sapiens chromosome 8 |
| | | SEQ = [G/A/ |
| | | >NC_000008.11: 11266947-11267446 |
| | | Homo sapiens chromosome 8 |
| rs7935393 | 2063 | >NC_000011.10: 128572704-128573203 |
| | | Homo sapiens chromosome 11 |
| | | SEQ = [A/C] |
| | | >NC_000011.10: 128573205-128573704 |
| | | Homo sapiens chromosome 11 |
| rs12934476 | 2064 | >NC_000016.10: 11237152-11237651 |
| | | Homo sapiens chromosome 16 |
| | | SEQ = [A/G] |
| | | >NC_000016.10: 11237653-11238152 |
| | | Homo sapiens chromosome 16 |
| rs12457255 | 2065 | >NC_000018.10: 12759477-12759976 |
| | | Homo sapiens chromosome 18 |
| | | SEQ = [C/A] |
| | | >NC_000018.10: 12759978-12760477 |
| | | Homo sapiens chromosome 18 |
| rs2070557 | 2066 | >NC_000021.9: 44234741-44235240 |
| | | Homo sapiens chromosome 21 |
| | | SEQ = [A/T] |
| | | >NC_000021.9: 44235242-44235741 |
| | | Homo sapiens chromosome 21 |
| rs4246905 | 2067 | >NC_000009.12: 114790469-114790968 |
| | | Homo sapiens chromosome 9 |
| | | SEQ = [T/A/ |
| | | >NC_000009.12: 114790970-114791469 |
| | | Homo sapiens chromosome 9 |
| rs10974900 | 2068 | >NC_000009.12: 4987458-4987957 |
| | | Homo sapiens chromosome 9 |
| | | SEQ = [C/T] |
| | | >NC_000009.12: 4987959-4988458 |
| | | Homo sapiens chromosome 9 |
| rs12434976 | 2069 | >NC_000014.9: 98185370-98185869 |
| | | Homo sapiens chromosome 14 |
| | | SEQ = [A/C] |
| | | >NC_000014.9: 98185871-98186370 |
| | | Homo sapiens chromosome 14 |
| rs16901748 | 2070 | >NC_000005.10: 11561609-11562108 |
| | | Homo sapiens chromosome 5 |
| | | SEQ = [G/T] |
| | | >NC_000005.10: 11562110-11562609 |
| | | Homo sapiens chromosome 5 |
| rs2815844 | 2071 | >NC_000001.11: 241083704-241084203 |
| | | Homo sapiens chromosome 1 |
| | | SEQ = [C/T] |
| | | >NC_000001.11: 241084205-241084704 |
| | | Homo sapiens chromosome 1 |
| rs889702 | 2072 | >NC_000016.10: 6088639-6089138 |
| | | Homo sapiens chromosome 16 |
| | | SEQ = [G/A] |
| | | >NC_000016.10: 6089140-6089639 |
| | | Homo sapiens chromosome 16 |
| rs2409750 | 2073 | >NC_000008.11: 11229685-11230184 |
| | | Homo sapiens chromosome 8 |
| | | SEQ = [A/C] |
| | | >NC_000008.11: 11230186-11230685 |
| | | Homo sapiens chromosome 8 |
| rs1541020 | 2074 | >NC_000010.11: 6122567-6123066 |
| | | Homo sapiens chromosome 10 |
| | | SEQ = [C/T] |
| | | >NC_000010.11: 6123068-6123567 |
| | | Homo sapiens chromosome 10 |
| rs4942248 | 2075 | >NC_000013.11: 43832169-43832668 |
| | | Homo sapiens chromosome 13 |
| | | SEQ = [T/A] |
| | | >NC_000013.11: 43832670-43833169 |
| | | Homo sapiens chromosome 13 |
| rs12934476 | 2076 | >NC_000016.10: 11237152-11237651 |
| | | Homo sapiens chromosome 16 |
| | | SEQ = [A/G] |
| | | >NC_000016.10: 11237653-11238152 |
| | | Homo sapiens chromosome 16 |
| rs12457255 | 2077 | >NC_000018.10: 12759477-12759976 |
| | | Homo sapiens chromosome 18 |
| | | SEQ = [C/A] |
| | | >NC_000018.10: 12759978-12760477 |
| | | Homo sapiens chromosome 18 |
| rs2297437 | 2078 | >NC_000020.11: 63673421-63673920 |
| | | Homo sapiens chromosome 20 |
| | | SEQ = [G/A] |
| | | >NC_000020.11: 63673922-63674421 |
| | | Homo sapiens chromosome 20 |
| rs41309367 | 2079 | >NC_000020.11: 63677701-63678200 |
| | | Homo sapiens chromosome 20 |
| | | SEQ = [C/T] |
| | | >NC_000020.11: 63678202-63678701 |
| | | Homo sapiens chromosome 20 |
| rs10733509 | 2080 | >NC_000009.12: 4307550-4308049 |
| | | Homo sapiens chromosome 9 |
| | | SEQ = [A/G] |
| | | >NC_000009.12: 4308051-4308550 |
| | | Homo sapiens chromosome 9 |
| rs10750376 | 2081 | >NC_000011.10: 127867534-127868033 |
| | | Homo sapiens chromosome 11 |
| | | SEQ = [C/T] |
| | | >NC_000011.10: 127868035-127868534 |
| | | Homo sapiens chromosome 11 |
| rs10932456 | 2082 | >NC_000002.12: 212988031-212988530 |
| | | Homo sapiens chromosome 2 |
| | | SEQ = [A/G] |
| | | >NC_000002.12: 212988532-212989031 |
| | | Homo sapiens chromosome 2 |
| rs1326860 | 2083 | >NC_000001.11: 193834579-193835078 |
| | | Homo sapiens chromosome 1 |
| | | SEQ = [A/G] |
| | | >NC_000001.11: 193835080-193835579 |
| | | Homo sapiens chromosome 1 |
| rs1528663 | 2084 | >NC_000011.10: 13945175-13945674 |
| | | Homo sapiens chromosome 11 |
| | | SEQ = [G/A] |
| | | >NC_000011.10: 13945676-13946175 |
| | | Homo sapiens chromosome 11 |
| rs1892231 | 2085 | >NC_000014.9: 98267730-98268229 |
| | | Homo sapiens chromosome 14 |
| | | SEQ = [A/C] |
| | | >NC_000014.9: 98268231-98268730 |
| | | Homo sapiens chromosome 14 |
| rs951279 | 2086 | >NC_000001.11: 208593050-208593549 |
| | | Homo sapiens chromosome 1 |
| | | SEQ = [A/G] |
| | | >NC_000001.11: 208593551-208594050 |
| | | Homo sapiens chromosome 1 |
| rs9806914 | 2087 | >NC_000016.10: 6097144-6097643 |
| | | Homo sapiens chromosome 16 |
| | | SEQ = [A/G] |
| | | >NC_000016.10: 6097645-6098144 |
| | | Homo sapiens chromosome 16 |
| rs7935393 | 2088 | >NC_000011.10: 128572704-128573203 |
| | | Homo sapiens chromosome 11 |
| | | SEQ = [A/C] |
| | | >NC_000011.10: 128573205-128573704 |
| | | Homo sapiens chromosome 11 |
| rs1690492 | 2089 | >NC_000016.10: 11224459-11224958 |
| | | Homo sapiens chromosome 16 |
| | | SEQ = [G/C] |
| | | >NC_000016.10: 11224960-11225459 |
| | | Homo sapiens chromosome 16 |
| rs420726 | 2090 | >NC_000021.9: 44239062-44239561 |
| | | Homo sapiens chromosome 21 |
| | | SEQ = [T/C] |
| | | >NC_000021.9: 44239563-44240062 |
| | | Homo sapiens chromosome 21 |
| rs7759385 | 2091 | >NC_000006.12: 106140395-106140894 |
| | | Homo sapiens chromosome 6 |
| | | SEQ = [T/A] |
| | | >NC_000006.12: 106140896-106141395 |
| | | Homo sapiens chromosome 6 |
| rs10974900 | 2092 | >NC_000009.12: 4987458-4987957 |
| | | Homo sapiens chromosome 9 |
| | | SEQ = [C/T] |
| | | >NC_000009.12: 4987959-4988458 |
| | | Homo sapiens chromosome 9 |
| rs1326860 | 2093 | >NC_000001.11: 193834579-193835078 |
| | | Homo sapiens chromosome 1 |
| | | SEQ = [A/G] |
| | | >NC_000001.11: 193835080-193835579 |
| | | Homo sapiens chromosome 1 |
| rs2548147 | 2094 | >NC_000005.10: 40151459-40151958 |
| | | Homo sapiens chromosome 5 |
| | | SEQ = [G/C] |
| | | >NC_000005.10: 40151960-40152459 |
| | | Homo sapiens chromosome 5 |
| rs2815844 | 2095 | >NC_000001.11: 241083704-241084203 |
| | | Homo sapiens chromosome 1 |
| | | SEQ = [C/T] |
| | | >NC_000001.11: 241084205-241084704 |
| | | Homo sapiens chromosome 1 |
| rs889702 | 2096 | >NC_000016.10: 6088639-6089138 |
| | | Homo sapiens chromosome 16 |
| | | SEQ = [G/A] |
| | | >NC_000016.10: 6089140-6089639 |
| | | Homo sapiens chromosome 16 |
| rs9806914 | 2097 | >NC_000016.10: 6097144-6097643 |
| | | Homo sapiens chromosome 16 |
| | | SEQ = [A/C/ |
| | | >NC_000016.10: 6097645-6098144 |
| | | Homo sapiens chromosome 16 |
| rs6478109 | 2098 | >NC_000009.12: 114805986-114806485 |
| | | Homo sapiens chromosome 9 |
| | | SEQ = [A/G] |
| | | >NC_000009.12: 114806487-114806986 |
| | | Homo sapiens chromosome 9 |
| rs7278257 | 2099 | >NC_000021.9: 44233381-44233880 |
| | | Homo sapiens chromosome 21 |
| | | SEQ = [G/C] |
| | | >NC_000021.9: 44233882-44234381 |
| | | Homo sapiens chromosome 21 |
| rs11221332 | 2100 | >NC_000011.10: 128510579-128511078 |
| | | Homo sapiens chromosome 11 |
| | | SEQ = [C/A/ |
| | | >NC_000011.10: 128511080-128511579 |
| | | Homo sapiens chromosome 11 |
| rs56124762 | 2101 | >NC_000021.9: 44238091-44238590 |
| | | Homo sapiens chromosome 21 |
| | | SEQ = [A/G] |
| | | >NC_000021.9: 44238592-44239091 |
| | | Homo sapiens chromosome 21 |
| rs2070558 | 2102 | >NC_000021.9: 44235275-44235774 |
| | | Homo sapiens chromosome 21 |
| | | SEQ = [G/A] |
| | | >NC_000021.9: 44235776-44236275 |
| | | Homo sapiens chromosome 21 |
| rs2070561 | 2103 | >NC_000021.9: 44237587-44238086 |
| | | Homo sapiens chromosome 21 |
| | | SEQ = [T/C] |
| | | >NC_000021.9: 44238088-44238587 |
| | | Homo sapiens chromosome 21 |
| rs7134599 | 2104 | >NC_000012.12: 68105795-68106294 |
| | | Homo sapiens chromosome 12 |
| | | SEQ = [G/A] |
| | | >NC_000012.12: 68106296-68106795 |
| | | Homo sapiens chromosome 12 |
| rs6062496 | 2105 | >NC_000020.11: 63697246-63697745 |
| | | Homo sapiens chromosome 20 |
| | | SEQ = [G/A] |
| | | >NC_000020.11: 63697747-63698246 |
| | | Homo sapiens chromosome 20 |
| rs7468800 | 2106 | >NC_000009.12: 114829225-114829724 |
| | | Homo sapiens chromosome 9 |
| | | SEQ = [C/A] |
| | | >NC_000009.12: 114829726-114830225 |
| | | Homo sapiens chromosome 9 |
| rs1569328 | 2107 | >NC_000014.9: 75274548-75275047 |
| | | Homo sapiens chromosome 14 |
| | | SEQ = [C/T] |
| | | >NC_000014.9: 75275049-75275548 |
| | | Homo sapiens chromosome 14 |
| rs2284553 | 2108 | >NC_000021.9: 33403889-33404388 |
| | | Homo sapiens chromosome 21 |
| | | SEQ = [A/G] |
| | | >NC_000021.9: 33404390-33404889 |
| | | Homo sapiens chromosome 21 |
| rs6062504 | 2357 | >NC_000020.11: 63717055-63717554 |
| | | Homo sapiens chromosome 20 |
| | | SEQ = [A/G/ |
| | | >NC_000020.11: 63717556-63718055 |
| | | Homo sapiens chromosome 20 |
| rs7556897 | 2358 | >NC_000002.12: 227794896-227795395 |
| | | Homo sapiens chromosome 2 |
| | | SEQ = [C/G/ |
| | | >NC_000002.12: 227795397-227795896 |
| | | Homo sapiens chromosome 2 |

### 7.6 Example 6. Validation of 3-SNP Models for TL1A Companion Diagnostic

The machine learning workflow identified several SNP model combinations for the development of the TL1A companion diagnostic (TL1A CDx). Previous analyses had identified 3-SNP combination models composed of variants associated with TNFSF15 (rs6478109), ICOSLG (rs7278257, rs2070557), ETS1 (rs7935393), and RBFOX1 (rs9806914) genes as well as variant rs1892231. These SNP models were identified via a Cedars Sinai Crohn's Disease cohort. In order to validate the findings, an external cohort of Crohn's Disease patients was identified and genotyped. Genotype and TL1A protein expression were obtained from the non-Cedars cohort in order to validate the 3-SNP models. A total of 712 Crohn's Disease individuals were genotyped while a 114 subset of the 712 samples were used to obtain TL1A protein expression via PBMC assays.

### Genotyping and Imputation of Cohort

The initial data mining analyses was performed on a Cedars Sinai cohort using genotypes from the Illumina's ICHIP (Immunochip) platform. The validation cohort was genotyped using Illumina's GSA platform (24v2.0), which has substantial improvements over the Immunochip platform. Most of the SNPs identified in the models were not present on the GSA platform. Therefore, imputation of the GSA genotype data was initiated in order to obtain a larger panel of genotyped SNPs so the SNP models could be validated. Genotype imputation refers to the prediction of genotypes that are not directly assayed on a given platform. Different methodologies and significant improvements in algorithms have been developed for implementing genotype imputation. The quality of the genotype imputation was validated by selecting a random sample of 120 patients from our validation cohort to be genotyped for a small set of imputed SNPs. The overall agreement between imputed genotype and assay genotypes were evaluated using Cohen's Kappa statistic.

Validation cohort genotyped results were downloaded from Illumina and transformed into PED format (through Illumina's Genome Studio Workbench) so that they could be further processed using the PLINK software (v1.9). The genotyped data went through a process of QC looking at factors such as heterozygosity, SNP missingness, MAF distributions, and relatedness of samples in order to prepare the genotype data for ancestry determination. Once genotyped data was QCed, the admixture and ancestry PCA plots were used to determine which samples were of European (EUR) ancestry to move forward with imputation. In order to perform imputation, ancestry needs to be taken into account, since genotype reference panels based on ancestry are used by imputation algorithms. For our imputation, the European Reference Panel: hrc.r1.1 for the reference panel was selected. All qc'd EUR ancestry genotyped samples were submitted to the Michigan Imputation Server for genotype imputation. The resulting imputed genotypes were downloaded and further processed for model validation.

In order to move forward with analyzing the imputed genotypes for the 3-SNP model validation, a random selection of 120 samples from the initial 470 samples were sent to Illumina for genotyping in order to compare the imputed genotype with the actual laboratory genotype results. The imputed genotypes were evaluated by looking at the level of agreement for the following SNPs: rs6478109, rs2070557, rs1892231, rs7935393.

The SNP rs6478109 was already on the GSA platform and this was used as a “control” to determine that the assay was in fact working appropriately. The levels of agreement (based on Cohen's Kappa) were high (based on the table below) and therefore it was decided to move forward with using the imputed genotype data for further analysis.

TABLE 6
Levels of Agreement Based On Cohen's Kappa

| SNP | Rsq | Cohen's Kappa |
|---|---|---|
| rs6478109 | 0.998 | 1.00 |
| rs2070557 | 0.978 | 0.98 |
| rs1892231 | 0.989 | 1.00 |
| rs7935393 | 0.980 | 1.00 |

### Evaluation and Validation of 3-SNP Models

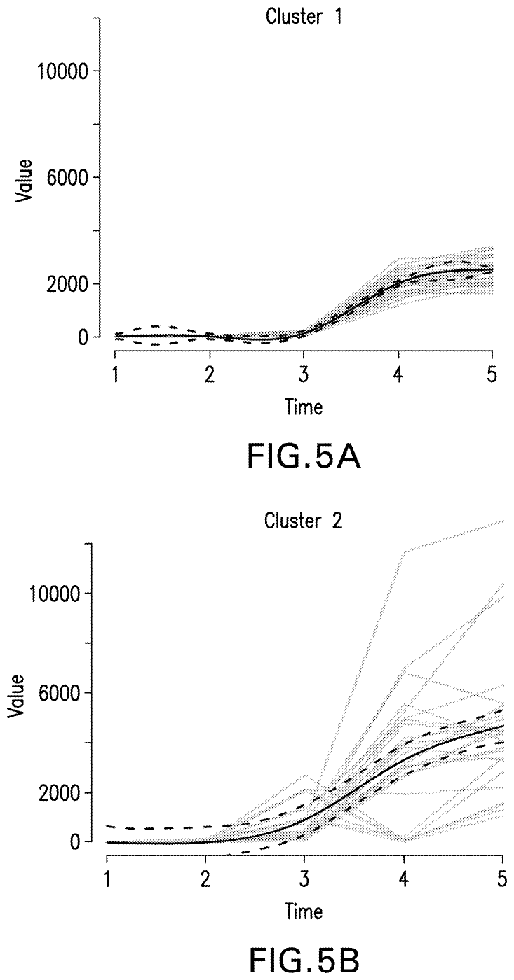

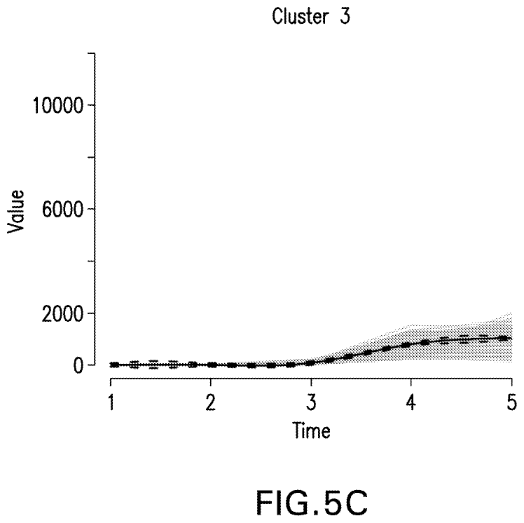

Next, the initial 3-SNP models were evaluated using the validation cohort. Similar to the analysis of the Cedars cohort TL1A protein expression data, the TL1A Expression (based on PBMC assay) from the validation cohort was clustered using the TL1A measurements at the 0, 3, 6, 24, and 72 hour time points. The clustering identified 3 cluster within the dataset, which are provided in FIG. 5A-5C. FIG. 5A shows cluster 1, FIG. 5B shows cluster 2, and FIG. 5C shows cluster 3.

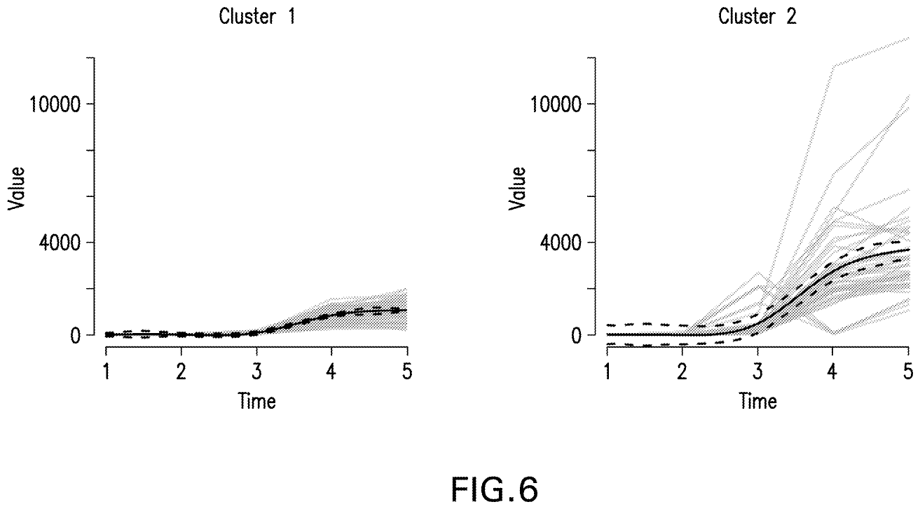

The 3 clusters were collapsed into 2 clusters (high expression clusters) and (low expression clusters), which are shown in FIG. 6. The clusters above were collapsed down to two clusters because there was substantial overlap between cluster 3 (FIG. 5C) above and cluster 1 (FIG. 6, left). The same level of overlap was seen for clusters 1 (FIG. 5A) and 2 (FIG. 5B) (based on the 3 clusters) and cluster 2 (FIG. 6, right).

The imputed genotype data were integrated from the validation cohort with the TL1A protein expression data in order to determine which models validated in the external validation cohort. This was done by looking at results of the TL1A CDx 3-SNP model. The 3-SNP models were evaluated in the context of the validation cohort and the TL1A expression clusters. “Positive” genotype hits were determined based on the association of a particular genotype and its ability to associate with the higher expressing TL1A cluster. Without being bound by any particular theory, high TL1A clusters (e.g., high expression of TL1A in CD patients, relative to baseline expression of TL1A in normal individuals) directly correlates with positive therapeutic responsiveness to an inhibitor of TL1A activity or expression; whereas low TL1A clusters (e.g., low TL1A expression in CD patients, relative to baseline expression of TL1A in normal individuals) directly correlates to non-responsiveness to an inhibitor of TL1A activity or expression.

Calculations of Positive Predictive Value (PPV) and Specificity were calculated for the 3-SNP models. Each model consists of 3 unique SNPs based off of the identified SNPs in our training cohort analysis (TNFSF15 (rs6478109), ICOSLG (rs7278257, rs2070557), ETS1 (rs7935393), and RBFOX1 (rs9806914) genes as well as variant rs1892231). The PPV and Specificity for Models A-C are provided in Table 7. As shown in Table 7, Model A may be used to predict a positive therapeutic response to a treatment with an inhibitor of TL1A activity or expression with a PPV of at least or about 0.797 and a specificity of at least or about 0816 (when considering both training and validation cohorts combined). Model A was further explored due to its better performance compared to Models B and C (when considering both training and validation cohorts combined).

TABLE 7
Exemplary 3-SNP Models A-C

| | | | Validation | | | | |
|---|---|---|---|---|---|---|---|
| | Training Cohort | | Cohort | | Combined | | CD |
| Model | PPV | SPEC | PPV | SPEC | PPV | SPEC | FREQ |
| A | 0.902 | 0.867 | 0.643 | 0.783 | 0.797 | 0.816 | 38.7 |
| B | 0.806 | 0.767 | 0.682 | 0.848 | 0.759 | 0.816 | 26.3 |
| C | 0.838 | 0.800 | 0.576 | 0.696 | 0.714 | 0.737 | 36.7 |

Other previously identified 3-SNP models were further evaluated due the fact that only Model A showed strong concordance of positive hit genotypes across the training and validation cohorts. These additional SNP models (Models D-K) consisted of 3-SNP combinations of the following SNPs: rs6478109, rs7935393, rs9806914, rs16901748, rs2070557, rs7278257, rs2297437, rs1892231, as shown in Table 8.

TABLE 8
3-SNP Models D-K

| | Cohort | | Cohort | | | | |
|---|---|---|---|---|---|---|---|
| | 1 | | 2 | | Combined | | CD |
| Model | PPV | SPEC | PPV | SPEC | PPV | SPEC | FREQ |
| D | 0.815 | 0.833 | 0.750 | 0.848 | 0.782 | 0.842 | 18.6 |
| E | 0.848 | 0.833 | 0.606 | 0.717 | 0.727 | 0.763 | 31.2 |
| F | 0.909 | 0.933 | 0.667 | 0.870 | 0.800 | 0.895 | 18.3 |
| G | 0.917 | 0.933 | 0.609 | 0.804 | 0.766 | 0.855 | 22.6 |
| H | 0.923 | 0.967 | 0.727 | 0.935 | 0.833 | 0.947 | 12.9 |
| I | 0.941 | 0.967 | 0.733 | 0.913 | 0.844 | 0.934 | 17.2 |
| J | 0.923 | 0.967 | 0.727 | 0.935 | 0.833 | 0.947 | 12.9 |
| K | 0.844 | 0.833 | 0.731 | 0.848 | 0.793 | 0.842 | 23.9 |

As before, the PPV and Specificity for each model was evaluated in the context of positive and negative hits and their association with high and lower TL1A clusters. After evaluating the models across both training and validation cohorts, an additional model (Model K) was evaluated, which contained SNP, rs16901748 (CTNND2), which had not been utilized in our previous models (based on the initial training cohort).

### ICOSLG Proxy SNP Selection

One of ICOSLG SNPs (rs7278257) was determined to be challenging to genotype given the SNP location within the genome. Therefore, candidate proxy SNPs to rs7278257 were identified. The proxy SNPs were identified via LDLink. A list of potential proxy SNPs for rs7278257 is shown below in Table 9.

TABLE 9
Proxy SNPs Utilized in Validation

| RS Number | Coord | Alleles | MAF | Distance | Dprime | R2 |
|---|---|---|---|---|---|---|
| rs7278257 | chr21: 45653764 | (G/C) | 0.2833 | 0 | 1 | 1 |
| rs56124762 | chr21: 45658474 | (A/G) | 0.2763 | 4710 | 0.9849 | 0.9372 |
| rs11558819 | chr21: 45656774 | (C/T) | 0.2873 | 3010 | 0.9705 | 0.9236 |
| rs2070557 | chr21: 45655124 | (A/T) | 0.2972 | 1360 | 0.9651 | 0.8705 |
| rs2070558 | chr21: 45655658 | (G/A) | 0.2952 | 1894 | 0.9602 | 0.87 |
| rs2329718 | chr21: 45656199 | (T/C) | 0.2952 | 2435 | 0.9602 | 0.87 |
| rs2070559 | chr21: 45657700 | (C/G) | 0.2982 | 3936 | 0.96 | 0.8573 |
| rs2070560 | chr21: 45657848 | (C/G) | 0.2972 | 4084 | 0.9551 | 0.8526 |
| rs2070561 | chr21: 45657970 | (T/C) | 0.2972 | 4206 | 0.9551 | 0.8526 |

Next, the SNPs provided in Table 9 were analyzed for relevant inflammatory bowel disease related clinical associations within the Cedars R/Shiny database. From the SNPs above, the following SNPs had relevant clinical associations (examples include key phenotypes: CD vs. Ctrl, IBD vs. Ctrl,L1 B2a+B2b vs B1): rs2070561, rs56124762, rs2070558, rs11558819. CD v. Ctrl refers to cases of Crohn's disease versus cases of controls (individuals without Crohn's disease); IBD vs. Ctrl refers to cases of inflammatory bowel disease versus cases of controls (individuals without IBD); L1 refers to the ileum; B2a+B2b refers to stricturing and penetrating disease; B1 refers to non-stricturing and non-penetrating disease.

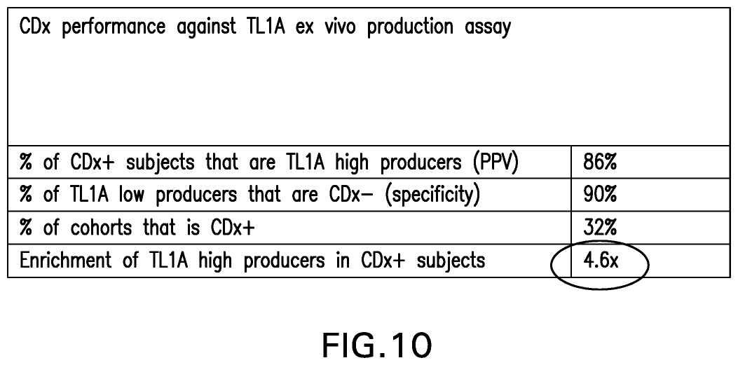

The TL1A companion diagnostic (CDx) described herein was validated in two independent CD and UC patient cohorts, the results of which are summarized in FIG. 10. The TL1A CDx captured approximately 32% of the IBD population and predicted with positive predictive value (PPV) of 86% whether the CD or the UC subjects were positive for a therapeutic response to TL1A (“CDx+”). Overall, the TL1A CDx had an approximately 4.6× greater probability of identifying patients predisposed to increased TL1A expression (“high producers”) over IBD patients predisposed to lowered TL1A expression (“low producers”). Across two CD patient cohorts alone, the TL1A CDx predicted as high as 42% of the IBD patients to have increased TL1A expression.

### 7.7 Example 7. Validation in Japanese Cohort

An external cohort of Crohn's Disease patients of Japanese ancestry is identified and genotyped. Genotype and TL1A protein expression are obtained from second Japanese cohort in order to validate the 3-SNP models. A total of 800 Crohn's Disease individuals are genotyped while a 100 subset of the 800 samples are used to obtain TL1A protein expression via PBMC assays.

The initial 3-SNP models are evaluated using the Japanese validation cohort. Similar to the analysis of the validation cohort in Example 6, the TL1A Expression (based on PBMC assay) from the validation cohort is clustered using the TL1A measurements at the 0, 3, 6, 24, and 72 hour time points. The clustering identifies at least 2 clusters in the dataset: (i) high expression clusters and (ii) low expression clusters.

The imputed genotype data are integrated from the Japanese validation cohort with the TL1A protein expression data in order to determine which models validated in the Japanese validation cohort. This is done by looking at results of the TL1A CDx 3-SNP model. The 3-SNP models are evaluated in the context of the Japanese validation cohort and the TL1A expression clusters. “Positive” genotype hits are determined based on the association of a particular genotype and its ability to associate with the higher expressing TL1A cluster. Calculations of PPV and Specificity are calculated for the 3-SNP models. Each model consists of 3 unique SNPs based off of the identified SNPs in the training cohort analysis (TNFSF15 (rs6478109), ICOSLG (rs7278257, rs2070557), ETS1 (rs7935393), and RBFOX1 (rs9806914) genes as well as variant rs1892231). The 3-SNP models shown in Table 7 and Table 8 are explored in this validation. The PPV and SPEC values expected in the Japanese validation cohort for Models A-K are the same as those reported in Table 7 and op. Without being bound by any particular theory, validation of the 3-SNP models for the TL1A CDx is expected across all ancestral populations.

Candidate proxy SNPs to any one of the SNPs provided in Table 7 or Table 8 may be identified. The proxy SNPs are identified via LDLink using a reference population of Japanese ancestry. The proxy SNPs are further analyzed for relevant inflammatory bowel disease related clinical associations within the Cedars R/Shiny database in Japanese cohorts, such as for example, CD vs. Ctrl, IBD vs. Ctrl,L1 B2a+B2b vs B1).

### 7.8 Example 8: Design of Humanized Anti-TL1A Antibodies

Two different strategies were employed to identify humanized variants that express well in mammalian cells, preserve TL1A binding, and display high monomeric content.

The first strategy utilized a previously humanized variant, termed ASX, that displays high monomeric content (98%) and expresses well (30 μg/mL in small-scale transient cultures) as a template for additional mutagenesis. However, ASX contains a significant number of murine framework residues, eight heavy chain residues and 7 light chain residues, that may pose an immunogenicity risk. The ASX heavy and light chain templates were used to systematically mutate murine framework residues to human residues corresponding to the most closely related human germline framework. The goal of this strategy was to reduce the total number of murine framework residues while preserving the favorable expression and solubility characteristics of ASX. Because ASX contained 15 murine framework residues there were 2\hat{ }15 (32,768) distinct variants (restricting each position to either the murine or the human residue) that could be made and tested.

The second strategy utilized a previously humanized variant, termed c34, that expresses well (17 μg/mL in small-scale transient cultures) and contains CDRs optimized for binding within a fully human germline framework, as a template for additional mutagenesis. Large-scale expression of c34 unexpectedly resulted in a sub-optimal monomeric content (55-60%). The c34 heavy and light chain templates were used to systematically mutate certain framework residues to murine residues corresponding to the original murine antibody framework. The goal of this strategy was to improve the solubility of c34 (monomeric content) through the introduction of as few murine framework residues as possible (minimizing potential immunogenicity risks) while preserving the favorable expression characteristics of c34.

For both strategies, the initial approach was to scan differing framework residues, one at a time, and express and characterize the variants. Thus, human framework residues were introduced into variant ASX where it differed from c34 and conversely, murine framework mutations were introduced into variant c34 where it differed from ASX. The initial scan identified certain framework and CDR residues that had minimal impact on the characteristics displayed by the template antibody while other mutations had a more dramatic impact, favorable in some cases and unfavorable in others. The information gained from the positional scan was subsequently used in an iterative and combinatorial fashion, to identify multiple variants with favorable characteristics. Importantly, by applying a stepwise, iterative and combinatorial approach the beneficial variants were identified without necessitating the expression and characterization of 32,768 distinct variants.

In certain cases, mutation of the first residue of the heavy chain from glutamine to aspartic acid or glutamic acid was evaluated, alone or in combination with other mutations.

In addition, for both strategies certain CDR residues were also mutated to determine the impact on expression and solubility. For example, a limited number of mutations in HCDR2, HCDR3 and LCDR3 were examined. Similar to the approach used with frameworks, the mutations were predominantly restricted to the original murine CDR residue or mutations that were previously identified as enhancing binding affinity.

Finally, for both strategies “shuffling” of heavy and light chains was used. Specifically, certain human light chains containing few murine framework residues and having a favorable impact on expression of antibody with higher monomeric content were identified early in the process and these were paired with various engineered heavy chains in order to accelerate the process of identifying suitable variants.

TABLE 10
Sequences of Certain Designed anti-TL1A Antibodies

| | Heavy Chain Variable | Light Chain Variable |
|---|---|---|
| Antibody | Region SEQ ID NO | Region SEQ ID NOS |
| A15 | 108 | 203 |
| A29 | 108 | 205 |
| A30 | 108 | 204 |
| A31 | 136 | 205 |
| A32 | 137 | 205 |
| A33 | 137 | 202 |
| A34 | 107 | 208 |
| A35 | 138 | 208 |
| A36 | 139 | 208 |
| A37 | 140 | 208 |
| A38 | 141 | 208 |
| A39 | 142 | 208 |
| A40 | 143 | 208 |
| A41 | 115 | 208 |
| A42 | 144 | 208 |
| A43 | 145 | 208 |
| A44 | 146 | 208 |
| A45 | 120 | 208 |
| A46 | 147 | 208 |
| A47 | 148 | 208 |
| A48 | 108 | 210 |
| A49 | 108 | 211 |
| A50 | 108 | 212 |
| A51 | 108 | 213 |
| A52 | 108 | 214 |
| A53 | 146 | 208 |
| A54 | 149 | 208 |
| A55 | 109 | 208 |
| A56 | 108 | 215 |
| A57 | 150 | 202 |
| A58 | 125 | 202 |
| A59 | 117 | 202 |
| A60 | 151 | 202 |
| A61 | 152 | 202 |
| A62 | 153 | 202 |
| A63 | 154 | 202 |
| A64 | 121 | 202 |
| A65 | 128 | 202 |
| A66 | 155 | 202 |
| A67 | 122 | 202 |
| A68 | 123 | 202 |
| A69 | 156 | 202 |
| A70 | 157 | 202 |
| A71 | 158 | 202 |
| A72 | 131 | 202 |
| A73 | 157 | 205 |
| A74 | 158 | 205 |
| A75 | 131 | 205 |
| A76 | 159 | 202 |
| A77 | 160 | 202 |
| A78 | 124 | 202 |
| A79 | 107 | 208 |
| A81 | 139 | 208 |
| A82 | 140 | 208 |
| A83 | 144 | 208 |
| A85 | 136 | 209 |
| A86 | 136 | 216 |
| A87 | 136 | 217 |
| A88 | 136 | 218 |
| A89 | 136 | 219 |
| A90 | 136 | 220 |
| A91 | 133 | 202 |
| A92 | 161 | 202 |
| A93 | 162 | 202 |
| A94 | 124 | 202 |
| A95 | 131 | 205 |
| A96 | 128 | 205 |
| A97 | 121 | 202 |
| A98 | 122 | 202 |
| A99 | 123 | 202 |
| A100 | 107 | 204 |
| A101 | 140 | 204 |
| A102 | 115 | 204 |
| A103 | 120 | 204 |
| A104 | 139 | 204 |
| A105 | 143 | 204 |
| A107 | 108 | 202 |
| A108 | 156 | 205 |
| A109 | 133 | 205 |
| A110 | 125 | 205 |
| A111 | 150 | 205 |
| A112 | 117 | 205 |
| A113 | 124 | 205 |
| A114 | 121 | 205 |
| A115 | 122 | 205 |
| A116 | 123 | 205 |
| A117 | 151 | 205 |
| A118 | 153 | 205 |
| A119 | 159 | 205 |
| A120 | 154 | 205 |
| A121 | 163 | 204 |
| A122 | 113 | 204 |
| A123 | 112 | 204 |
| A124 | 164 | 204 |
| A125 | 105 | 204 |
| A126 | 114 | 204 |
| A127 | 118 | 204 |
| A128 | 111 | 204 |
| A129 | 110 | 204 |
| A130 | 121 | 205 |
| A132 | 128 | 206 |
| A133 | 121 | 206 |
| A134 | 122 | 206 |
| A135 | 133 | 206 |
| A136 | 125 | 206 |
| A137 | 121 | 207 |
| A138 | 122 | 207 |
| A139 | 110 | 207 |
| A140 | 110 | 202 |
| A141 | 111 | 207 |
| A142 | 111 | 202 |
| A143 | 136 | 202 |
| A144 | 111 | 204 |
| A145 | 133 | 201 |
| A146 | 125 | 201 |
| A147 | 117 | 201 |
| A148 | 121 | 201 |
| A149 | 122 | 201 |
| A150 | 128 | 201 |
| A151 | 124 | 201 |
| A152 | 131 | 201 |
| A153 | 133 | 205 |
| A154 | 125 | 205 |
| A155 | 121 | 205 |
| A156 | 122 | 205 |
| A157 | 104 | 204 |
| A158 | 101 | 204 |
| A159 | 119 | 204 |
| A160 | 102 | 204 |
| A161 | 165 | 204 |
| A162 | 106 | 204 |
| A163 | 166 | 204 |
| A164 | 167 | 204 |
| A165 | 139 | 205 |
| A166 | 146 | 205 |
| A167 | 120 | 205 |
| A168 | 147 | 205 |
| A169 | 126 | 205 |
| A170 | 135 | 205 |
| A171 | 168 | 205 |
| A172 | 130 | 205 |
| A173 | 127 | 205 |
| A174 | 132 | 205 |
| A175 | 126 | 201 |
| A176 | 135 | 201 |
| A177 | 168 | 201 |
| A178 | 130 | 201 |
| A179 | 127 | 201 |
| A180 | 132 | 201 |
| A181 | 107 | 202 |
| A182 | 138 | 202 |
| A183 | 140 | 202 |
| A184 | 145 | 202 |
| A185 | 147 | 202 |
| A186 | 144 | 202 |
| A187 | 120 | 202 |
| A188 | 115 | 202 |
| A189 | 146 | 202 |
| A190 | 141 | 202 |
| A191 | 142 | 202 |
| A192 | 143 | 202 |
| A193 | 109 | 205 |
| A194 | 103 | 205 |
| A195 | 169 | 205 |
| A196 | 129 | 205 |
| A197 | 116 | 205 |
| A198 | 134 | 205 |
| A199 | 109 | 201 |
| A200 | 103 | 201 |
| A201 | 169 | 201 |
| A202 | 129 | 201 |
| A203 | 116 | 201 |
| A204 | 134 | 201 |
| A205 | 109 | 202 |
| A206 | 103 | 202 |
| A207 | 169 | 202 |
| A208 | 129 | 202 |
| A209 | 116 | 202 |
| A210 | 134 | 202 |
| A211 | 108 | 201 |
| A212 | 107 | 201 |
| A213 | 106 | 201 |
| A214 | 111 | 201 |
| A215 | 110 | 201 |
| A216 | 112 | 201 |
| A217 | 101 | 201 |
| A218 | 119 | 201 |
| A219 | 104 | 201 |
| A220 | 102 | 205 |
| A221 | 105 | 201 |
| A222 | 114 | 201 |
| A223 | 103 | 202 |
| A224 | 116 | 201 |
| A500 | 301 | 303 |
| A501 | 302 | 303 |

As used herein, reference to A(number), refers to an antibody of this table. For instance, A15 used herein refers to A15 in Table 10.

### 7.9 Example 9: Generation and Characterization of Humanized Anti-tl1a Antibodies

Humanized anti-TL1A antibodies designed in Example 1 were prepared and characterized.

### Cloning of Humanized Antibodies

DNA encoding leader sequence and the heavy and light chain variable regions of humanized variants of interest was cloned into pFuse1-hIgG1-Fc1 (InvivoGen) and pFuse2-CLig-hk (InvivoGen), respectively. Two distinct humanized heavy chain templates, termed ASX-HC and c34-HC, and four distinct humanized light chain templates, termed ASX-LC, cH3-1, c34-LC, cXL3-13-LC and cXL3-15-LC were all cloned.

In order to introduce mutations into the templates, the QuickChange Site Directed Mutagenesis Kit (Agilent, cat. #200518) was used per manufacturer's directions. Briefly, mutagenesis was performed using miniprep double-stranded plasmid DNA, two synthetic oligonucleotides primers containing the desired mutation, PfuTurbo® DNA polymerase and a temperature cycler. Following temperature cycling, the product was treated with Dpn I. The nicked vector DNA containing the mutation(s) of interest was used to transform bacteria. Subsequently, colonies were picked, the DNA was sequenced to confirm mutagenesis and was subsequently used for transfection of mammalian FreeStyle 293-F cells.

### Antibody Expression

Small-scale (3 mL, 6-well) expression of variants in FreeStyle 293-F cells was performed in the following manner. One or two days prior to transfection cells were passaged so that the density would be >1×10⁶ cells/mL on the day of the transfection. Typically, this meant passaging at 6-7×105 cells/mL one day prior or 4×105 cells/mL two days prior. Transfections were only performed with cell viability>90%. On the day of the transfection Opti-MEM media was warmed to 37° C. and cells were resuspended to 1.1×106 cells/mL, using 3.3×106 cells per 3 mL transfection. A total of 3 ug DNA was used for each transfection. Briefly, the transfections used heavy and light chain plasmid at a heavy chain:light chain ratio of 1:3. For 3 mL transfections, 4 μL 293fectin was added to 96 μL Opti-MEM, combined with 100 μL DNA mixture, and incubated at 25° C. for 20-30 minutes. Subsequently, this mixture was added dropwise to 2.8 mL cells and the plate was transferred to an incubator and placed on a rotating platform at 175 rpm for up to 120 hours. After 96-120 hours, transfection supernatants were collected by centrifuging the transfected cells and supernatant at 1200 rpm for 5 min. The supernatant was transferred to a clean tube and centrifuged again at 3900 rpm for 10 min to remove any remaining cell debris. The supernatant was filtered through a 0.45 mm PES syringe filter and stored at 4° C. until the next step.

### Quantitation of Antibody Expression

Antibody expression was quantitated by ELISA. Briefly, a Corning Costar 3366 96-well round bottom high bind plate was coated with 50 mL anti-kappa (2 μg/mL) in PBS overnight at 4° C. The plate was washed 3× with PBS-0.05% Tween 20 (PBS-T) and was blocked with 100 μL 10 BSA/PBS for 1 h at 25° C. The block was removed, and culture supernatant diluted 5-fold was added and serially diluted 2-fold across the plate. Every plate also contained an IgG standard diluted serially 3-fold beginning at 1 μg/mL. Samples were incubated for 1 h at 25° C., the plate was washed three times with PBS-T, and 50 μL anti-Fc HRP secondary (Southern Biotech #2048-05), diluted 1:4000 in BSA/PBS was added for 1 h at 25° C. The plate was washed three times with PBS-T and developed for up to 15 min following the addition of 50 μL Ultra TMB ELISA substrate (Thermo #34028). The reaction was terminated by the addition of 50 μL 2 N H₂SO₄ and the A450 nm was measured. Antibody expression levels obtained from 3 mL scale transfections are shown in Table 11.

TABLE 11
Expression, Binding, and Analytical SEC Characterization
of anti-TL1A Antibodies (ND, not determined)

| Var- | Expression | KD | % | Murine | HC | LC |
|---|---|---|---|---|---|---|
| iant | (ug/mL) | (pM) | Monomer | FR | Template | Template |
| 15 | 21 | ND | 87 | 8 | ASX | cH3-1 |
| 29 | 18 | 65 | 65 | 10 | ASX | c34 |
| 30 | 29 | 77 | 90 | 8 | ASX | cXL3-13 |
| 31 | 11 | 92 | 73 | 2 | c34 | c34 |
| 32 | 10 | 111 | 78 | 2 + D | c34 | c34 |
| 33 | 21 | 81 | 54 | 0 + D | c34 | c34 |
| 34 | 35 | <50 | 97 | 14 | ASX | ASX |
| 35 | 36 | 72 | 91 | 14 | ASX | ASX |
| 36 | 40 | <50 | 87 | 13 | ASX | ASX |
| 37 | 40 | 34 | 95 | 14 | ASX | ASX |
| 38 | 28 | 103 | 75 | 14 | ASX | ASX |
| 39 | 15 | 125 | 83 | 14 | ASX | ASX |
| 40 | 30 | <50 | 87 | 13 | ASX | ASX |
| 41 | 20 | 16 | 96 | 14 | ASX | ASX |
| 42 | 30 | <50 | 88 | 14 | ASX | ASX |
| 43 | 18 | 51 | 90 | 14 | ASX | ASX |
| 44 | ND | ND | ND | 13 | ASX | ASX |
| 45 | 15 | 85 | 90 | 13 | ASX | ASX |
| 46 | 27 | 63 | 72 | 13 | ASX | ASX |
| 47 | 18 | 82 | 78 | 12 | ASX | ASX |
| 48 | 22 | 76 | 92 | 14 | ASX | ASX |
| 49 | 26 | 92 | 65 | 13 | ASX | ASX |
| 50 | 33 | 19 | 94 | 14 | ASX | ASX |
| 51 | 16 | <50 | 93 | 14 | ASX | ASX |
| 52 | 29 | 27 | 91 | 13 | ASX | ASX |
| 53 | 26 | 126 | 84 | 13 | ASX | ASX |
| 54 | 25 | 83 | 94 | 15 + D | ASX | ASX |
| 55 | 22 | 91 | 99 | 15 + E | ASX | ASX |
| 56 | 15 | 116 | 71 | 14 | ASX | ASX |
| 57 | 20 | 191 | 59 | 1 | c34 | c34 |
| 58 | 9 | 112 | 67 | 1 | c34 | c34 |
| 59 | 11 | 136 | 78 | 2 | c34 | c34 |
| 60 | 19 | 168 | 57 | 0 | c34 | c34 |
| 61 | 15 | 127 | 44 | 1 | c34 | c34 |
| 62 | 21 | 150 | 58 | 1 | c34 | c34 |
| 63 | 20 | 132 | 52 | 0 | c34 | c34 |
| 64 | 2 | 90 | 97 | 0 | c34 | c34 |
| 65 | 7 | 97 | 69 | 1 | c34 | c34 |
| 66 | 19 | 150 | 49 | 1 | c34 | c34 |
| 67 | 4 | 89 | 97 | 1 | c34 | c34 |
| 68 | 2 | 74 | 92 | 1 | c34 | c34 |
| 69 | 12 | 136 | 64 | 0 + E | c34 | c34 |
| 70 | 15 | 149 | 54 | 1 | c34 | c34 |
| 71 | 18 | 150 | 55 | 2 | c34 | c34 |
| 72 | 13 | 159 | 61 | 3 | c34 | c34 |
| 73 | 8 | 128 | 71 | 3 | c34 | c34 |
| 74 | 10 | 141 | 70 | 4 | c34 | c34 |
| 75 | 8 | 259 | 95 | 5 | c34 | c34 |
| 76 | 19 | ND | 50 | 0 | c34 | c34 |
| 77 | 12 | ND | 50 | 2 | c34 | c34 |
| 78 | 3 | ND | 86 | 2 | c34 | c34 |
| 79 | 42 | ND | 98 | 14 | ASX | ASX |
| 81 | 31 | ND | 88 | 13 | ASX | ASX |
| 82 | 26 | ND | 92 | 14 | ASX | ASX |
| 83 | 29 | ND | 74 | 14 | ASX | ASX |
| 85 | 25 | 130 | 49 | 1 | c34 | c34 |
| 86 | 26 | 129 | 55 | 1 | c34 | c34 |
| 87 | 26 | 121 | 52 | 1 | c34 | c34 |
| 88 | 9 | 81 | 63 | 2 | c34 | c34 |
| 89 | 31 | 117 | 55 | 1 | c34 | c34 |
| 90 | 19 | 107 | 53 | 1 | c34 | c34 |
| 91 | 14 | 132 | 63 | 1 | c34 | c34 |
| 92 | 20 | 121 | 49 | 1 | c34 | c34 |
| 93 | 12 | 117 | 63 | 2 | c34 | c34 |
| 94 | 5 | 81 | 91 | 2 | c34 | c34 |
| 95 | 13 | 105 | 92 | 5 | c34 | c34 |
| 96 | 7 | 95 | 99 | 3 | c34 | c34 |
| 97 | 2 | 71 | 97 | 0 | c34 | c34 |
| 98 | 7 | 140 | 98 | 1 | c34 | c34 |
| 99 | 3 | 102 | 95 | 1 | c34 | c34 |
| 100 | 39 | 84 | 84 | 7 | ASX | cXL3-13 |
| 101 | 23 | 96 | 81 | 7 | ASX | cXL3-13 |
| 102 | 19 | 104 | 75 | 7 | ASX | cXL3-13 |
| 103 | 11 | 107 | 90 | 6 | ASX | cXL3-13 |
| 104 | 26 | 108 | 70 | 6 | ASX | cXL3-13 |
| 105 | 23 | 110 | 58 | 6 | ASX | cXL3-13 |
| 107 | 55 | 71 | 85 | 8 | ASX | c34 |
| 108 | 9 | 55 | 83 | 2 + E | c34 | c34 |
| 109 | 9 | 50 | 96 | 3 | c34 | c34 |
| 110 | 7 | 56 | 95 | 3 | c34 | c34 |
| 111 | 17 | 68 | 61 | 3 | c34 | c34 |
| 112 | 6 | 54 | 93 | 4 | c34 | c34 |
| 113 | 2 | 50 | 99 | 4 | c34 | c34 |
| 114 | 1 | 51 | 99 | 2 | c34 | c34 |
| 115 | 3 | 58 | 99 | 3 | c34 | c34 |
| 116 | 1 | 53 | 99 | 3 | c34 | c34 |
| 117 | 16 | 94 | 80 | 2 | c34 | c34 |
| 118 | 21 | 83 | 70 | 3 | c34 | c34 |
| 119 | 15 | 87 | 77 | 2 | c34 | c34 |
| 120 | 12 | 85 | 64 | 2 | c34 | c34 |
| 121 | 24 | 106 | 77 | 6 | ASX | cXL3-13 |
| 122 | 22 | 112 | 85 | 6 | ASX | cXL3-13 |
| 123 | 18 | 104 | 76 | 5 | ASX | cXL3-13 |
| 124 | 21 | 91 | 83 | 6 | ASX | cXL3-13 |
| 125 | 10 | 116 | 98 | 6 | ASX | cXL3-13 |
| 126 | 4 | 123 | 99 | 5 | ASX | cXL3-13 |
| 127 | 8 | 70 | 94 | 6 | ASX | cXL3-13 |
| 128 | 17 | 111 | 84 | 4 | ASX | cXL3-13 |
| 129 | 17 | 99 | 92 | 5 | ASX | cXL3-13 |
| 130 | 1 | 75 | 99 | 2 | c34 | c34 |
| 132 | 6 | 62 | 99 | 2 | c34 | cXL3-13 |
| 133 | 1 | 58 | 99 | 1 | c34 | cXL3-13 |
| 134 | 3 | 55 | 99 | 2 | c34 | cXL3-13 |
| 135 | 7 | 56 | 74 | 2 | c34 | cXL3-13 |
| 136 | 6 | 53 | 84 | 2 | c34 | cXL3-13 |
| 137 | 2 | 50 | 96 | 0 | c34 | cXL3-15 |
| 138 | 5 | 69 | 99 | 1 | c34 | cXL3-15 |
| 139 | 35 | 74 | 78 | 5 | ASX | cXL3-15 |
| 140 | 26 | 73 | 75 | 5 | ASX | c34 |
| 141 | 27 | 108 | 81 | 4 | ASX | cXL3-15 |
| 142 | 25 | 126 | 68 | 4 | ASX | c34 |
| 143 | 16 | 85 | 57 | 0 | c34 | c34 |
| 144 | ND | ND | ND | 4 | ASX | cXL3-13 |
| 145 | 20 | 70 | 78 | 2 | c34 | c34 |
| 146 | 25 | 65 | 84 | 2 | c34 | c34 |
| 147 | 26 | 63 | 87 | 3 | c34 | c34 |
| 148 | 2 | 46 | 98 | 1 | c34 | c34 |
| 149 | 7 | 48 | 99 | 2 | c34 | c34 |
| 150 | 15 | 59 | 83 | 2 | c34 | c34 |
| 151 | 5 | 57 | 96 | 3 | c34 | c34 |
| 152 | 36 | 58 | 73 | 4 | c34 | c34 |
| 153 | 9 | 49 | 97 | 3 | c34 | c34 |
| 154 | 8 | 66 | 92 | 3 | c34 | c34 |
| 155 | 1 | 67 | 99 | 2 | c34 | c34 |
| 156 | 2 | 94 | 99 | 3 | c34 | c34 |
| 157 | 6 | 69 | 93 | 4 | ASX | cXL3-13 |
| 158 | 6 | 66 | 91 | 3 | ASX | cXL3-13 |
| 159 | 4 | 69 | 99 | 4 | ASX | cXL3-13 |
| 160 | 7 | 94 | 99 | 4 | ASX | cXL3-13 |
| 161 | 11 | 72 | 59 | 4 | ASX | cXL3-13 |
| 162 | 9 | 75 | 79 | 3 | ASX | cXL3-13 |
| 163 | 22 | 51 | 60 | 4 | ASX | cXL3-13 |
| 164 | 23 | 58 | 61 | 4 | ASX | cXL3-13 |
| 165 | 19 | 59 | 53 | 8 | ASX | c34 |
| 166 | 13 | 57 | 76 | 8 | ASX | c34 |
| 167 | 9 | 42 | 96 | 8 | ASX | c34 |
| 168 | 16 | 62 | 85 | 8 | ASX | c34 |
| 169 | 8 | 47 | 90 | 3 | c34 | c34 |
| 170 | 9 | 49 | 93 | 3 | c34 | c34 |
| 171 | 13 | 50 | 80 | 5 | c34 | c34 |
| 172 | 7 | 40 | 96 | 3 | c34 | c34 |
| 173 | 4 | 40 | 99 | 4 | c34 | c34 |
| 174 | 4 | 43 | 98 | 4 | c34 | c34 |
| 175 | 31 | 45 | 86 | 2 | c34 | c34 |
| 176 | 18 | 48 | 80 | 2 | c34 | c34 |
| 177 | 35 | 52 | 67 | 4 | c34 | c34 |
| 178 | 18 | 43 | 85 | 2 | c34 | c34 |
| 179 | 16 | 79 | 93 | 3 | c34 | c34 |
| 180 | 17 | 58 | 94 | 3 | c34 | c34 |
| 181 | 46 | 60 | 87 | 7 | ASX | c34 |
| 182 | 39 | 67 | 74 | 7 | ASX | c34 |
| 183 | 38 | 65 | 82 | 7 | ASX | c34 |
| 184 | 30 | 61 | 73 | 7 | ASX | c34 |
| 185 | 30 | 56 | 66 | 6 | ASX | c34 |
| 186 | 38 | 67 | 66 | 7 | ASX | c34 |
| 187 | 27 | 56 | 72 | 6 | ASX | c34 |
| 188 | 31 | 63 | 87 | 7 | ASX | c34 |
| 189 | 44 | 76 | 71 | 6 | ASX | c34 |
| 190 | 32 | 57 | 69 | 7 | ASX | c34 |
| 191 | 21 | 57 | 80 | 7 | ASX | c34 |
| 192 | 27 | 55 | 70 | 6 | ASX | c34 |
| 193 | 16 | 55 | 68 | 10 + E | ASX | c34 |
| 194 | 16 | 51 | 87 | 9 + E | ASX | c34 |
| 195 | 12 | 56 | 82 | 5 + E | c34 | c34 |
| 196 | 7 | 54 | 97 | 3 + E | c34 | c34 |
| 197 | 7 | 54 | 97 | 3 + E | c34 | c34 |
| 198 | 9 | 53 | 95 | 3 + E | c34 | c34 |
| 199 | 28 | 50 | 93 | 9 + E | ASX | c34 |
| 200 | 24 | 52 | 99 | 8 + E | ASX | c34 |
| 201 | 25 | 58 | 82 | 4 + E | c34 | c34 |
| 202 | 13 | 59 | 87 | 2 + E | c34 | c34 |
| 203 | 18 | 62 | 89 | 2 + E | c34 | c34 |
| 204 | 11 | 53 | 84 | 2 + E | c34 | c34 |
| 205 | 27 | 55 | 86 | 8 + E | ASX | c34 |
| 206 | 20 | 50 | 98 | 7 + E | ASX | c34 |
| 207 | ND | ND | ND | 3 + E | c34 | c34 |
| 208 | ND | ND | ND | 1 + E | c34 | c34 |
| 209 | 14 | 58 | 66 | 1 + E | c34 | c34 |
| 210 | 15 | 70 | 61 | 1 + E | c34 | c34 |
| 211 | 42 | 58 | 96 | 9 | ASX | c34 |
| 212 | 33 | 50 | 99 | 8 | ASX | c34 |
| 213 | 29 | 49 | 99 | 4 | ASX | c34 |
| 214 | 27 | 51 | 97 | 5 | ASX | c34 |
| 215 | 20 | 48 | 77 | 6 | ASX | c34 |
| 216 | 24 | 49 | 97 | 6 | ASX | c34 |
| 217 | 15 | 43 | 99 | 4 | ASX | c34 |
| 218 | 13 | 51 | 96 | 5 | ASX | c34 |
| 219 | 21 | 50 | 99 | 5 | ASX | c34 |
| 220 | 18 | 50 | 99 | 6 | ASX | c34 |
| 221 | 23 | 51 | 98 | 7 | ASX | c34 |
| 222 | 29 | 60 | 96 | 6 | ASX | c34 |
| 223 | 19 | 62 | 98 | 7 + E | ASX | c34 |
| 224 | 15 | 76 | 92 | 2 + E | c34 | c34 |

### Antibody Binding to Human TL1A

Antibody binding to human TL1A (Fitzgerald 930R-AT070) was quantitated by ELISA. Briefly, a Corning Costar 3366 96-well round bottom high bind plate was coated with 50 μL TL1A (1 μg/mL) in PBS overnight at 4° C. The plate was washed 3× with PBS-0.05% Tween 20 (PBS-T) and was blocked with 100 μL 1% BSA/PBS for 1 h at 25° C. The block was removed, and culture supernatant diluted 5-fold was added and serially diluted 2-fold across the plate. Samples were incubated for 1 h at 25° C., the plate was washed three times with PBS-T, and 50 μL anti-Fc HRP secondary, diluted 1:4000 in BSA/PBS was added for 1 h at 25° C. The plate was washed three times with PBS-T and developed for up to 15 min following the addition of 50 μL Ultra TMB ELISA substrate. The reaction was terminated by the addition of 50 μL 2 N H₂SO₄ and the A450 nm was measured. The antibody affinities, as determined by ELISA titration against human TL1A using unpurified culture supernatants, is shown in Table 11.

### Purification of Antibodies

Antibodies were purified from culture supernatants in a single step using Dynabeads Protein A (ThermoFisher Scientific, cat. #10002D). First, culture supernatants were concentrated per manufacturer's instructions using an Amicon Ultra-4 Centrifugal Filter Unit (30,000 MWCO; Millipore Sigma, cat. #C7719). The Dynabeads were resuspended by gentle vortexing and 100 μL were transferred to an Eppendorf tube. Using a magnet to retain the beads, the storage buffer was removed, and the beads were washed with 0.5 mL of 20 mM sodium phosphate, 150 mM NaCl, pH 7.4 (EB, Equilibration Buffer). A total of up to 24 μg of IgG from culture supernatant was added to the beads and mixed gently until the beads were resuspended. When necessary, antibody supernatants were diluted with EB. The tubes were placed sideways on a shaking platform and mixed for 10 min at 25° C. at 500 rpm. Subsequently, the beads were collected at the bottom of the tube using a microfuge at 10,000 rpm for 30 sec. Using a magnet to retain the beads, the supernatant was removed. The beads were washed once with 0.5 mL of 20 mM sodium phosphate, 500 mM NaCl, pH 7.4 followed by another wash with 50 mM sodium phosphate, pH 6.0. The beads were collected at the bottom of the tube using a microfuge at 10,000 rpm for 30 sec. Purified antibody was eluted from the beads using 20 uL 50 mM sodium acetate, pH 3.5 with gentle mixing for 2 min at 25° C. Using a magnet to retain the beads, the eluate was transferred to a fresh tube containing 1.1 uL 1 M Tris, pH 8.5 to neutralize the pH of the sample. This sample was then centrifuged at 10,000 rpm for 2 min and transferred to a fresh tube to ensure removal of residual Dynabeads. The concentration of the purified sample was determined using a DeNovix DS-11 Spectrophotometer/Fluorometer, buffer blank, and a mass extinction coefficient of 13.70 at 280 nm for a 1% IgG solution.

### Size Exclusion Chromatography

The antibodies were analyzed by size exclusion chromatography (SEC) to determine percent monomer and identify any large molecular weight aggregate contaminant species. A total volume of 15 μL of protein A purified antibodies at a concentration of 0.1-1 μg/L were analyzed using a Waters SEC column (Acquity UPLC BEH SEC, 200 Å, 1.7 m, 4.6×150 mm) on a Shimadzu UPLC instrument at a flow rate of 0.2 mL/min and a column oven temperature of 30° C. Standard PBS was used as the mobile phase and absorbance at 280 nm was used to monitor protein elution. For some antibody clones tested that demonstrated non-symmetrical elution profiles, PBS buffer supplemented with 350 mM NaCl at pH 6.0 was utilized to reduced non-specific interactions with the column matrix. The percent main peak (monomer) value was calculated using the Shimadzu software. Representative sample profiles are shown in FIGS. 7A-7C. The monomeric content of purified antibody variants is shown in Table 11.

### 7.10 Example 10: Abrogation of Effector Function

In certain cases, it might be beneficial to reduce the potential effector function of the antibodies. Multiple strategies to diminish effector function have been described, including point mutations to ablate FcγR and C1q binding, cross-subclass Fc designs to eliminate FcγR and C1q binding, and glycoengineering to ablate FcγR and C1q binding. Representative examples are highlighted in Table 12.

TABLE 12
Representative Aproaches to Abrogating Effector Function

| Mutation(s) | Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235A, G237A, P238S, H268A, | Decreases binding to FcγRI, IIa, IIb, IIIa |
| A330S, P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A, S | Diminished C1q binding |
| IgG2 | Decreases binding to FcγRs |
| IgG4 | Decreases binding to FcγRs; |
| | Does not activate complement system |
| S228P | Prevent IgG4 Fab arm exchange |
| S228P, F234A, L235A (IgG4) | Decreases binding to FcγRI, IIa, IIIa |
| IgG2-IgG4 cross-subclass (IgG2/G4) | Decreases binding to FcγRI, II, III, C1q |
| IgG2-IgG3 cross-subclass | Decreases binding to FcγRs; |
| | Decreases binding to C1q |
| H268Q, V309L, A330S, P331S (IgG2m4) | Decreases binding to FcγRI, II, III, C1q |
| V234A, G237A, P238S, H268A, V309L, | Decreases binding to FcγRI, IIa, IIb, IIIa, C1q |
| A330S, P331S (IgG2 σ) | |
| High mannose glycosylation | Decreases binding to C1q |

In order to express antibodies with abrogated effector function, the light chain variable regions of the antibodies disclosed in Example 2 and Table 10 are cloned with a kappa light chain constant region, while the heavy chain variable regions are cloned with a modified IgG1 heavy chain backbone, or a modified IgG2 backbone, or a modified IgG4 backbone, or an unmodified IgG2 or IgG4 backbone, such as those disclosed in Table 3 or elsewhere.

The impact of the various Fc engineering approaches on CDC activity can be assessed using C1q binding and C3 fixation assays. Purified antibodies are diluted in PBS and serial dilutions are plated on a microtiter plate for 12-18h at 4° C. The plates are blocked with 500 gelatin/PBS containing 100 (v/v) Tween-20 for 1 h at 25° C. Subsequently, the plates are incubated with 10% (v/v) human sera in PBS and C1q binding is detected using 1:500 dilution of HRP-conjugated rabbit anti-C1q (Bioss Inc.) in PBS containing 1% (v/v) Tween-20. To test C3 fixation, a 1:1000 dilution of rabbit anti C3 (abcam) is used followed by a 1:2000 dilution of HRP-conjugated chicken anti-rabbit IgG (abcam). The plates are developed as described for antibody quantitation assays in Example 1. EC50 values are calculated by fitting the data to a log (agonist) vs. response-variable slope (four parameter) model using GraphPad Prism (Sunnyvale, CA).

Additionally, the variants may be characterized for the binding of isolated C1q. MaxiSorp 384-well plates (Thermo Scientific, Nunc) are coated with serially diluted antibodies in 50 mM carbonate buffer, pH 9.6 (coat buffer), for 12-18h at 4° C. Plates are washed with phosphate buffered saline (PBS) containing 0.05% polysorbate 20, pH 7.4 and blocked with PBS containing 0.5% BSA, 0.05% polysorbate 20, 15 ppm Proclin and 10% Blocker Casein (ThermoScientific), pH 7.4. After 1-hour incubation at 25° C., plates are washed. Human C1q (Quidel, San Diego, CA) in the same buffer is added and incubated for 1.5 hour. Bound C1q is detected by adding 20 ng/mL biotinylated mouse anti-mouse C1q (Hycult biotech, cross reacting with human C1q) for 1.5 hour followed by horseradish peroxidase (HRP)-conjugated streptavidin (GE Healthcare Life Sciences) for 1 hour. To check for coating efficiency, some coated wells receive buffer only for the first two incubation steps and receive goat anti-human Fab′2-HRP when the wells used for measuring C1q binding received streptavidin-HRP. Plates are washed after each incubation step. Peroxidase activity is detected with substrate 3, 3′, 5, 5′-tetramethyl benzidine (TMB) (Kirkegaard & Perry Laboratories). The reaction is stopped with 1M phosphoric acid and absorbance is measured at 450 nm. Dose-response binding curves are fitted with a four-parameter model and EC50 values are calculated using GraphPad Prism (Sunnyvale, CA).

The impact of the various Fc engineering approaches on ADCC activity is assessed using soluble FcγR receptor binding ELISAs. Soluble human FcγRI, FcγRIIb and FcγRIII (binding affinity to both the F158 and V158 polymorphic forms of FcγRIII is assessed) are expressed as recombinant fusion proteins with Gly-His6-glutathione-S-transferase (GST) at the C-terminus of the extracellular domain of the receptor. MaxiSorp 384-well plates are coated with 1 μg/ml human FcγR in coat buffer. Plates are washed and blocked with PBS containing 0.5% BSA, 15 ppm Proclin, pH 7.4. After a 1 h incubation, plates are washed and 3-fold serial dilution of antibodies in PBS containing 0.5% BSA, 0.05% polysorbate 20, 15 ppm Proclin, pH 7.4 is added to the plates and incubated for 2 h. For enhanced binding sensitivity due to avidity, immune complexes are formed using anti-human antibody. Bound antibody is detected with HRP-conjugated goat anti-human kappa (Southern Biotech) using Ultra TMB substrate as described in Example 1. The reaction is terminated and the plate is read as described above. The dose-dependent binding curve of the wild type antibody (no Fc modifications) is fitted with GraphPad Prism (Sunnyvale, CA) four parameter curve fitting program. The relative affinity of the variant vs. the wild type is estimated by dividing the equivalent ng/ml wild type concentration at the appropriate concentration.

In addition, the variants are tested directly in Fc effector bioassays (Promega) following manufacturer's directions. These assays include FcγRIIa-H ADCP Bioassay (Promega cat #G9901), ADCC Reporter Bioassays, FcγRIIIa F Variant (Promega, cat #G9798), ADCC Reporter Bioassays, FeγRIIIa V Variant (Promega, cat, #G7015). The variants are tested both as monomeric Ig and as small immune complexes (ICs) by using an anti-hu Ig antibody to form small ICs.

A Europium based ADCC assay is performed. Briefly, peripheral blood lymphocytes (PBLs) are isolated by Ficoll Paque Plus gradient centrifugation. The PBLs are collected, washed with RPMI1640, 10% FCS and resuspended in cell culture medium. The cells are diluted to 2.5×10⁶ cells/ml. Target cells are labelled with BADTA (2,2′:6′,2″-terpyridine-6,6″-dicarboxylic acid acetoxymethylester): Cells are harvested by adding Accutase (Millipore), washed once and diluted to 1×10⁶ cells/ml. Next, 2.5 uL BADTA is added per 1×10⁶ cells and incubated for 35 min at 37° C. with 5% CO₂. After labelling the cells are diluted with 10 ml culture medium, centrifuged at 200×g for 10 min and supernatant aspirated. This step is repeated 3× with culture medium/2 mM Probenicid and the sample is diluted to 1×10⁵ cells/ml, centrifuged at 300×g for 5 min, supernatant taken off and 50 μL pipetted into the wells intended for the background controls. The final ratio of effector (PBL) to target cells is 25:1.

Controls include: (1) Background: the 50 μL aliquot, diluted with 100 μL medium, (2) Spontaneous lysis: 50 μL of the labelled target cell suspension plus 100 μL culture medium, incubated 2 h at 37° C., (3) Maximal lysis: 50 μL/well of the labelled target cell suspension plus 100 μL Triton X-100 (0.5% in PBS) incubated 2 h at 37° C., (4) Lysis control without antibodies: 50 μL/well of the labelled target cell suspension and 50 μL culture medium plus 50 μL of effector cells incubated 2 h at 37° C., (5) Lysis control without effector cells: 50 μL/well of the labelled target cell suspension; add 50 μL culture medium plus antibody at highest concentration used and incubate 2 h at 37° C.

At the end of the incubation period the 96 well plate is centrifuged at 100 rpm. 20 μL of each supernatant is transferred into an OptiPlate HTRF-96 (Packard) and 200 μL Europium solution is added and incubated for 15 min on a shaker. Fluorescence is measured as for time resolved fluorescence and spontaneous release and specific release are calculated.

A CDC assay is performed. Briefly, target cells are washed and diluted to 1×10⁵ cells/ml and 100 μL/well (10⁴ cells) are added to a 96-well flat bottom microtiter plate. A titration curve of the test antibody is created using serial dilutions, beginning at 1 μg/mL. Antibody is added to the plate, mixed gently, and is then placed at 37° C./5% CO2 incubator for 30 min. Next, 25 μL freshly dissolved baby rabbit complement (Cedarlane CL3441, 1 ml lyophilized, dilute freshly in 4 ml double distilled water) is added, mixed gently, and the plate is incubated at 37° C./5% CO2 incubator for 30 min. After the incubation period 50 μL supernatant is taken off and 100 μL Cell Titer Glo. reagent (Promega Corp.) is added to the remaining 100 μL supernatant. The plate is placed on an orbital shaker for 2 min, 100 μL/well is transferred into a black luminescence microtiter plate (Costar) and luminescence is measured. Controls included: (1) medium control (target cells plus 50 μL medium), (2) maximal lysis control (target cells plus 50 μL 0.5% Triton X-100), (3) complement control (target cells plus 25 μL medium plus 25 μL complement).

### 7.11 Example 11: Characterization of Potency and Species Selectivity in Whole Blood Assay

The relative potency of a panel of candidate antibodies was first assessed by determining the inhibition of interferon gamma release in human blood using the antibodies at 1 and 10 nM. All of the antibodies displayed potent activity, with A219 appearing to be one of the most potent candidates (Table 13).

TABLE 13

| Clone | % Inhibition at 1 nM Ig | % Inhibition at 10 nM Ig |
|---|---|---|
| A147 | 51.3 | 72.4 |
| A212 | 46.8 | 71.2 |
| A213 | 48.6 | 69.8 |
| A217 | 46.0 | 72.2 |
| A219 | 59.8 | 75.2 |
| A220 | 36.9 | 63.2 |

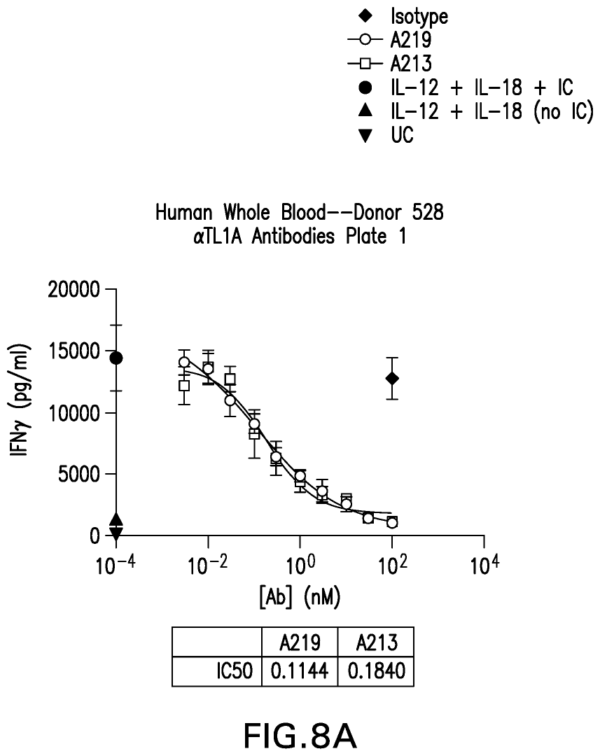

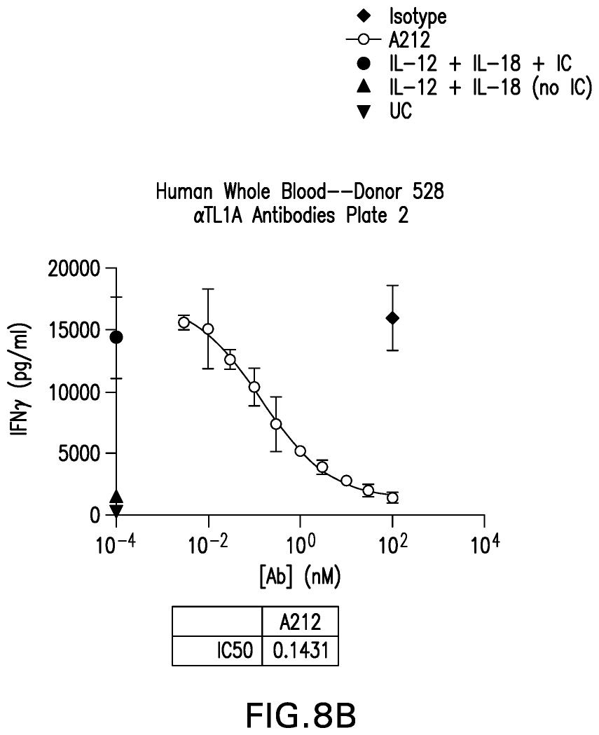

Next, three of the variants were characterized for inhibition of interferon gamma release in human blood using multiple human blood donors and testing the antibodies across a broader range of concentrations (0.01-100 nM). Representative inhibition profiles of variants A212, A213 and A219 are shown in FIG. 8. The mean IC50 values for these variants, and a control antibody termed 1D1, for the inhibition of interferon gamma release from multiple human donors is shown in Table 14.

TABLE 14

| Clone | Mean | SD |
|---|---|---|
| A212 | 51.3 | 72.4 |
| A213 | 46.8 | 71.2 |
| A219 | 48.6 | 69.8 |
| 1D1 | 46.0 | 72.2 |

### 7.12 Example 12: In Vivo Assessment of Anti-Tl1a Efficacy

The efficacy of anti-TL1A antibodies in animal models of colitis is performed. Anti-TL1A antibodies are tested in rodent models of acute colitis induced by intrarectal administration of di- or tri-nitrobenzenesulfonic acid (D/TNBS) or oxazolone, and chronic colitis induced by administration of DSS in drinking water or transfer of CD45RB^{hi} T cells. DNBS and oxazolone induce localized ulceration and inflammation. DSS administration induces robust generalized inflammation of the intestinal tract characterized by erosive lesions and inflammatory infiltrate. Symptoms of all these models usually include diarrhea, occult blood, weight loss and occasionally rectal prolapse. In a prophylactic model, antibody treatment begins at the start of administration of the colitis-inducing compound. In a therapeutic model, antibody treatment begins several days after commencement of induction. The effect of the treatment on weight, stool consistency and occult blood, as well as microscopic effects on epithelial integrity and degree of inflammatory infiltrate is determined. Daily clinical scoring is performed based on stool consistency and presence of occult blood giving a disease activity index (DAI) score.

### 7.13 Example 13: Phase I Clinical Trial

A phase 1 clinical trial is performed to evaluate the safety, tolerability, pharmacokinetics and pharmacodynamics of an anti-TL1A antibody from Table 20 on subjects having Crohn's disease (CD).

Single ascending dose (SAD) arms: Subjects in each group (subjects are grouped based on the presence of a genotype comprising at least one, and preferably three, polymorphism(s) selected from Table 1 and subjects without the presence of the genotype) receive either a single dose of the antibody or a placebo. Exemplary doses are 1, 3, 10, 30, 100, 300, 600 and 800 mg of antibody. Safety monitoring and PK assessments are performed for a predetermined time. Based on evaluation of the PK data, and if the antibody is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group (subjects are grouped based on the same criteria as above) receive multiple doses of the antibody or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Inclusion Criteria: Healthy subjects of non-childbearing potential between the ages of 18 and 55 years. Healthy is defined as no clinically relevant abnormalities identified by a detailed medical history, full physical examination, including blood pressure and pulse rate measurement, 12 lead ECG and clinical laboratory tests. Female subjects of non-childbearing potential must meet at least one of the following criteria: (1) achieved postmenopausal status, defined as: cessation of regular menses for at least 12 consecutive months with no alternative pathological or physiological cause; and have a serum follicle stimulating hormone (FSH) level within the laboratory's reference range for postmenopausal females; (2) have undergone a documented hysterectomy and/or bilateral oophorectomy; (3) have medically confirmed ovarian failure. All other female subjects (including females with tubal ligations and females that do NOT have a documented hysterectomy, bilateral oophorectomy and/or ovarian failure) will be considered to be of childbearing potential. Body Mass Index (BMI) of 17.5 to 30.5 kg/m2; and a total body weight>50 kg (110 lbs). Evidence of a personally signed and dated informed consent document indicating that the subject (or a legal representative) has been informed of all pertinent aspects of the study.

Two groups of CD patients are selected: patients having the genotype described herein, and patients without the genotype. For example, the genotype may comprise rs6478109, rs56124762, and rs1892231; rs6478109, rs56124762, and rs16901748; rs6478109, rs1892231, and rs16901748; rs56124762, rs1892231, and rs16901748; rs6478109, rs2070558, and rs1892231; rs6478109, rs2070558, and rs16901748; rs6478109, rs1892231, and rs16901748; rs2070558, rs1892231, and rs16901748; rs6478109, rs2070561, and rs1892231; rs6478109, rs2070561, and rs16901748; rs6478109, rs1892231, and rs16901748, rs2070561, rs1892231, and rs16901748; rs6478109, rs7935393, and rs1892231; rs6478109, rs7935393, and rs9806914; rs6478109, rs7935393, and rs7278257; rs6478109, rs7935393, and rs2070557; rs6478109, rs1892231, and rs9806914; rs6478109, rs1892231, and rs7278257; rs6478109, rs1892231, and rs2070557; rs6478109, rs9806914, and rs7278257; rs6478109, rs9806914, and rs2070557; rs6478109, rs7278257, and rs2070557; rs7935393, rs1892231, and rs9806914; rs7935393, rs1892231, and rs7278257; rs7935393, rs1892231, and rs2070557; rs7935393, rs9806914, and rs7278257; rs7935393, rs9806914, and rs2070557; rs7935393, rs7278257, and rs2070557; rs1892231, rs9806914, and rs7278257; rs1892231, rs9806914, and rs2070557; rs1892231, rs7278257, and rs2070557; or rs9806914, rs7278257, and rs2070557.

Exclusion Criteria: Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing). Subjects with a history of or current positive results for any of the following serological tests: Hepatitis B surface antigen (HBsAg), Hepatitis B core antibody (HBcAb), anti-Hepatitis C antibody (HCV Ab) or human immunodeficiency virus (HIV). Subjects with a history of allergic or anaphylactic reaction to a therapeutic drug. Treatment with an investigational drug within 30 days (or as determined by the local requirement, whichever is longer) or 5 half-lives or 180 days for biologics preceding the first dose of study medication. Pregnant females; breastfeeding females; and females of childbearing potential.

Primary Outcome Measures: Incidence of dose limiting or intolerability treatment related adverse events (AEs) [Time Frame: 12 weeks]. Incidence, severity and causal relationship of treatment emergent AEs (TEAEs) and withdrawals due to treatment emergent adverse events [Time Frame: 12 weeks]. Incidence and magnitude of abnormal laboratory findings [Time Frame: 12 weeks]. Abnormal and clinically relevant changes in vital signs, blood pressure (BP) and electrocardiogram (ECG) parameters [Time Frame: 12 weeks].

Secondary Outcome Measures: Single Ascending Dose: Maximum Observed Plasma Concentration (Cmax) [Time Frame: 12 weeks]. Single Ascending Dose: Time to Reach Maximum Observed Plasma Concentration (Tmax) [Time Frame: 12 weeks]. Single Ascending Dose: Area under the plasma concentration-time profile from time zero to 14 days (AUC14 days) [Time Frame: 12 weeks]. Single Ascending Dose: Area under the plasma concentration-time profile from time zero extrapolated to infinite time (AUCinf) [Time Frame: 12 weeks]. Single Ascending Dose: Area under the plasma concentration-time profile from time zero to the time of last quantifiable concentration (AUClast) [Time Frame: 12 weeks]. Single Ascending Dose: Dose normalized maximum plasma concentration (Cmax[dn]) [Time Frame: 12 weeks]. Single Ascending Dose: Dose normalized area under the plasma concentration-time profile from time zero extrapolated to infinite time (AUCinf[dn]) [Time Frame: 12 weeks]. Single Ascending Dose: Dose normalized area under the plasma concentration-time profile from time zero to the time of last quantifiable concentration (AUClast[dn]) [Time Frame: 12 weeks]. Single Ascending Dose: Plasma Decay Half-Life (t½) [Time Frame: 12 weeks]. Plasma decay half-life is the time measured for the plasma concentration to decrease by one half. Single Ascending Dose: Mean residence time (MRT) [Time Frame: 12 weeks]. Single Ascending Dose: Volume of Distribution at Steady State (Vss) [Time Frame: 6 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Single Ascending Dose: Systemic Clearance (CL) [Time Frame: 6]. CL is a quantitative measure of the rate at which a drug substance is removed from the body.

Multiple Ascending Dose First Dose: Maximum Observed Plasma Concentration (Cmax) [Time Frame: 12 weeks]. Multiple Ascending Dose First Dose: Time to Reach Maximum Observed Plasma Concentration (Tmax) [Time Frame: 12 weeks]. Multiple Ascending Dose First Dose: Area under the plasma concentration-time profile from time zero to time τ, the dosing interval where τ=2 weeks (AUCτ) [Time Frame: 12 weeks]. Multiple Ascending Dose First Dose: Dose normalized maximum plasma concentration (Cmax[dn]) [Time Frame: 12 weeks]. Multiple Ascending Dose First Dose: Dose normalized Area under the plasma concentration-time profile from time zero to time τ, the dosing interval where τ=2 weeks (AUCτ [dn]) [Time Frame: 12 weeks]. Plasma Decay Half-Life (t½) [Time Frame: 12 weeks]. Plasma decay half-life is the time measured for the plasma concentration to decrease by one half. Multiple Ascending Dose First Dose: Mean residence time (MRT) [Time Frame: 12 weeks]. Apparent Volume of Distribution (Vz/F) [Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose First Dose: Volume of Distribution at Steady State (Vss) [Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Multiple Ascending Dose First Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance is estimated from population pharmacokinetic (PK) modeling. Drug clearance is a quantitative measure of the rate at which a drug substance is removed from the blood. Multiple Ascending Dose First Dose: Systemic Clearance (CL) [Time Frame: 12 weeks]. CL is a quantitative measure of the rate at which a drug substance is removed from the body.

Multiple Ascending Dose Multiple Dose: Maximum Observed Plasma Concentration (Cmax) [Time Frame: 12 weeks]. Multiple Ascending Dose Multiple Dose: Time to Reach Maximum Observed Plasma Concentration (Tmax) [Time Frame: 12 weeks]. Multiple Ascending Dose Multiple Dose: Area under the plasma concentration-time profile from time zero to time τ, the dosing interval where τ=2 weeks (AUCτ) [Time Frame: 12 weeks]. Multiple Ascending Dose Multiple Dose: Dose normalized maximum plasma concentration (Cmax[dn]) [Time Frame: 12 weeks]. Multiple Ascending Dose Multiple Dose: Dose normalized Area under the plasma concentration-time profile from time zero to time τ, the dosing interval where τ=2 weeks (AUCτ [dn]) [Time Frame: 12 weeks]. Multiple Ascending Dose Multiple Dose: Plasma Decay Half-Life (t½) [Time Frame: 12 weeks]. Plasma decay half-life is the time measured for the plasma concentration to decrease by one half. Multiple Ascending Dose Multiple Dose: Apparent Volume of Distribution (Vz/F) [Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose Multiple Dose: Volume of Distribution at Steady State (Vss) [Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state.

Multiple Ascending Dose Multiple Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance was estimated from population pharmacokinetic (PK) modeling. Drug clearance is a quantitative measure of the rate at which a drug substance is removed from the blood. Multiple Ascending Dose Multiple Dose: Systemic Clearance (CL) [Time Frame: 12 weeks]. CL is a quantitative measure of the rate at which a drug substance is removed from the body. Multiple Ascending Dose Multiple Dose: Minimum Observed Plasma Trough Concentration (Cmin) [Time Frame: 12 weeks]. Multiple Ascending Dose Multiple Dose: Average concentration at steady state (Cav) [Time Frame: 12 weeks]. Multiple Ascending Dose Multiple Dose: Observed accumulation ratio (Rac) [Time Frame: 12 weeks]. Multiple Ascending Dose Multiple Dose: Peak to trough fluctuation (PTF) [Time Frame: 12 weeks]. Multiple Ascending Dose Additional Parameter: estimate of bioavailability (F) for subcutaneous administration at the corresponding intravenous dose [Time Frame: 12 weeks]. Immunogenicity for both Single Ascending Dose and Multiple Ascending Dose: Development of anti-drug antibodies (ADA) [Time Frame: 12 weeks].

### 7.14 Example 14: Phase 1B Clinical Trial

A phase 1b open label clinical trial is performed to evaluate efficacy of an anti-TL1A antibody on subjects having CD.

Arms: 10 patients positive for genotypes comprising at least one, but preferably three, polymorphism(s) provided in Table 1 are administered the antibody. 10 patients negative for the genotype are administered the antibody. Patients are monitored in real-time. Central ready of endoscopy and biopsy is employed, with readers blinded to point of time of treatment and endpoints.

For example, the genotypes may comprise rs6478109, rs56124762, and rs1892231; rs6478109, rs56124762, and rs16901748; rs6478109, rs1892231, and rs16901748; rs56124762, rs1892231, and rs16901748; rs6478109, rs2070558, and rs1892231; rs6478109, rs2070558, and rs16901748; rs6478109, rs1892231, and rs16901748; rs2070558, rs1892231, and rs16901748; rs6478109, rs2070561, and rs1892231; rs6478109, rs2070561, and rs16901748; rs6478109, rs1892231, and rs16901748; rs2070561, rs1892231, and rs16901748; rs6478109, rs7935393, and rs1892231; rs6478109, rs7935393, and rs9806914; rs6478109, rs7935393, and rs7278257; rs6478109, rs7935393, and rs2070557; rs6478109, rs1892231, and rs9806914; rs6478109, rs1892231, and rs7278257; rs6478109, rs1892231, and rs2070557; rs6478109, rs9806914, and rs7278257; rs6478109, rs9806914, and rs2070557; rs6478109, rs7278257, and rs2070557; rs7935393, rs1892231, and rs9806914; rs7935393, rs1892231, and rs7278257; rs7935393, rs1892231, and rs2070557; rs7935393, rs9806914, and rs7278257; rs7935393, rs9806914, and rs2070557; rs7935393, rs7278257, and rs2070557; rs1892231, rs9806914, and rs7278257; rs1892231, rs9806914, and rs2070557; rs1892231, rs7278257, and rs2070557; or rs9806914, rs7278257, and rs2070557.

Inclusion Criteria: Two groups of patients are selected: subject with the genotype described herein, and patients without the genotype.

Primary Outcome Measures: Simple Endoscopic Score for Crohn's Disease (SESCD), Crohn's Disease Activity Index (CDAI), and Patient Reported Outcome (PRO). If risk either positive group shows 50% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### 7.15 Example 15: Phase 2A Clinical Trial

A phase 2a clinical trial is performed to evaluate the efficacy of an anti-TL1A antibody in patients with CD. Optionally, the patients are positive for a genotype comprising at least one, but preferably three, polymorphism(s) provided in Table 1.

Arms: 40 patients per arm (antibody and placebo arms) are treated with antibody or placebo for 12 weeks. An interim analysis is performed after 20 patients from each group are treated at the highest dose to look for a 40-50% delta between placebo and treated group in primary outcome (50% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### 7.16 Example 16: Treating Crohn's Disease (CD) in a Subject

CD is treated in a subject, by first, determining the genotypes of the subject. Optionally, the subject is, or is susceptible to, non-response to the induction of certain therapies such as anti-TNF, steroids, or immunomodulators, or loses response to such therapies after a period of time. A sample of whole blood is obtained from the subject. An assay is performed on the sample obtained from the subject to detect a presence of a genotype comprising at least one, but preferably three or four, polymorphism(s) provided in Table 1.

At three polymorphisms comprising rs6478109, rs56124762, and rs1892231; rs6478109, rs56124762, and rs16901748; rs6478109, rs1892231, and rs16901748; rs56124762, rs1892231, and rs16901748; rs6478109, rs2070558, and rs1892231; rs6478109, rs2070558, and rs16901748; rs6478109, rs1892231, and rs16901748; rs2070558, rs1892231, and rs16901748; rs6478109, rs2070561, and rs1892231; rs6478109, rs2070561, and rs16901748; rs6478109, rs1892231, and rs16901748; rs2070561, rs1892231, and rs16901748; rs6478109, rs7935393, and rs1892231; rs6478109, rs7935393, and rs9806914; rs6478109, rs7935393, and rs7278257; rs6478109, rs7935393, and rs2070557; rs6478109, rs1892231, and rs9806914; rs6478109, rs1892231, and rs7278257; rs6478109, rs1892231, and rs2070557; rs6478109, rs9806914, and rs7278257; rs6478109, rs9806914, and rs2070557; rs6478109, rs7278257, and rs2070557; rs7935393, rs1892231, and rs9806914; rs7935393, rs1892231, and rs7278257; rs7935393, rs1892231, and rs2070557; rs7935393, rs9806914, and rs7278257; rs7935393, rs9806914, and rs2070557; rs7935393, rs7278257, and rs2070557; rs1892231, rs9806914, and rs7278257; rs1892231, rs9806914, and rs2070557; rs1892231, rs7278257, and rs2070557; or rs9806914, rs7278257, and rs2070557, or any of the above combinations in which a polymorphism is substituted with a proxy polymorphism, are detected in the sample by Illumina ImmunoArray or polymerase chain reaction (PCR) under standard hybridization conditions. Proxy polymorphisms are identified using linkage disequilibrium with a D′1 value of at least 0.8, or an r² value of at least 0.85. In some cases, the proxy polymorphism is additionally selected based on an independent association between the polymorphism and a relevant clinical phenotype of CD (e.g., stricturing and penetrating disease) In this example, one or more primer pairs described herein and/or nucleic acid probes comprising nucleic acid sequences capable of hybridizing the nucleic acid sequences, or their reverse compliments, provided in SEQ ID NOS: 2001-2041, or 2057-2059, are used.

A TNFSF15 profile is generated that correlates the presence or absence of the genotypes with a positive, negative or indeterminate result for a therapeutic response to treatment with an inhibitor of TL1A activity or expression with a positive predictive value and specificity of at least or about 70%.

The TNFSF15 profile of the subject is positive. Based on the TNFSF15 profile of the CD patient, a doctor determines that the subject is suitable for treatment with the inhibitor of TL1A activity or expression. A therapeutically effective amount of an inhibitor of TL1A activity or expression is administered to the subject with the positive TNFSF15 profile. The inhibitor of TL1A activity or expression may comprise an anti-TL1A antibody. The anti-TL1A antibody may be an antibody listed in Table 20. The anti-TL1A antibody may be a neutralizing anti-TL1A antibody.

TABLE 15
CDR Amino Acid Sequences

| SEQ ID | | |
|---|---|---|
| NO | Description | Sequence |
| 1 | P HCDR1 | GFDIQDTYMH |
| 601 | P HCDR1 | DTYMH |
| 602 | P HCDR1 | KYDIN |
| 603 | P HCDR1 | GFEIQDTYMH |
| 604 | P HCDR1 | GFDPQDTYMH |
| 605 | P HCDR1 | GFDVQDTYMH |
| 606 | P HCDR1 | GFDIGDTYMH |
| 607 | P HCDR1 | GFDISDTYMH |
| 608 | P HCDR1 | GFDIVDTYMH |
| 609 | P HCDR1 | GFDIQDAYMH |
| 610 | P HCDR1 | GFDIQDSYMH |
| 611 | P HCDR1 | GFDIQDTFMH |
| 612 | P HCDR1 | GFDIQDTYIH |
| 613 | P HCDR1 | GFDLQDTYMH |
| 614 | P HCDR1 | GFDIQDTYLH |
| 615 | P HCDR1 | GFDIGDTFIH |
| 616 | P HCDR1 | GFDIGDTFMH |
| 617 | P HCDR1 | GFDIGDTYIH |
| 618 | P HCDR1 | GFDIQDTFIH |
| 619 | P HCDR1 | GFDIQDTFMH |
| 620 | P HCDR1 | GFDIQDTYIH |
| 621 | P HCDR1 | GFDISDTFIH |
| 622 | P HCDR1 | GFDISDTFMH |
| 623 | P HCDR1 | GFDISDTYIH |
| 624 | P HCDR1 | GFDISDTYMH |
| 625 | P HCDR1 | GFDIVDTFIH |
| 626 | P HCDR1 | GFDIVDTFMH |
| 627 | P HCDR1 | GFDIVDTYIH |
| 628 | P HCDR1 | GFDPGDTFIH |
| 629 | P HCDR1 | GFDPGDTFMH |
| 630 | P HCDR1 | GFDPGDTYIH |
| 631 | P HCDR1 | GFDPGDTYMH |
| 632 | P HCDR1 | GFDPQDTFIH |
| 633 | P HCDR1 | GFDPQDTFMH |
| 634 | P HCDR1 | GFDPQDTYIH |
| 635 | P HCDR1 | GFDPQDTYMH |
| 636 | P HCDR1 | GFDPSDTFIH |
| 637 | P HCDR1 | GFDPSDTFMH |
| 638 | P HCDR1 | GFDPSDTYIH |
| 639 | P HCDR1 | GFDPSDTYMH |
| 640 | P HCDR1 | GFDPVDTFIH |
| 641 | P HCDR1 | GFDPVDTFMH |
| 642 | P HCDR1 | GFDPVDTYIH |
| 643 | P HCDR1 | GFDPVDTYMH |
| 644 | P HCDR1 | GFDVGDTFIH |
| 645 | P HCDR1 | GFDVGDTFMH |
| 646 | P HCDR1 | GFDVGDTYIH |
| 647 | P HCDR1 | GFDVGDTYMH |
| 648 | P HCDR1 | GFDVQDTFIH |
| 649 | P HCDR1 | GFDVQDTFMH |
| 650 | P HCDR1 | GFDVQDTYIH |
| 651 | P HCDR1 | GFDVSDTFIH |
| 652 | P HCDR1 | GFDVSDTFMH |
| 653 | P HCDR1 | GFDVSDTYIH |
| 654 | P HCDR1 | GFDVSDTYMH |
| 655 | P HCDR1 | GFDVVDTFIH |
| 656 | P HCDR1 | GFDVVDTFMH |
| 657 | P HCDR1 | GFDVVDTYIH |
| 658 | P HCDR1 | GFDVVDTYMH |
| 659 | P HCDR1 | GFEIGDTFIH |
| 660 | P HCDR1 | GFEIGDTFMH |
| 661 | P HCDR1 | GFEIGDTYIH |
| 662 | P HCDR1 | GFEIGDTYMH |
| 663 | P HCDR1 | GFEIQDTFIH |
| 664 | P HCDR1 | GFEIQDTFMH |
| 665 | P HCDR1 | GFEIQDTYIH |
| 666 | P HCDR1 | GFEIQDTYMH |
| 667 | P HCDR1 | GFEISDTFIH |
| 668 | P HCDR1 | GFEISDTFMH |
| 669 | P HCDR1 | GFEISDTYIH |
| 670 | P HCDR1 | GFEISDTYMH |
| 671 | P HCDR1 | GFEIVDTFIH |
| 672 | P HCDR1 | GFEIVDTFMH |
| 673 | P HCDR1 | GFEIVDTYIH |
| 674 | P HCDR1 | GFEIVDTYMH |
| 675 | P HCDR1 | GFEPGDTFIH |
| 676 | P HCDR1 | GFEPGDTFMH |
| 677 | P HCDR1 | GFEPGDTYIH |
| 678 | P HCDR1 | GFEPGDTYMH |
| 679 | P HCDR1 | GFEPQDTFIH |
| 680 | P HCDR1 | GFEPQDTFMH |
| 681 | P HCDR1 | GFEPQDTYIH |
| 682 | P HCDR1 | GFEPQDTYMH |
| 683 | P HCDR1 | GFEPSDTFIH |
| 684 | P HCDR1 | GFEPSDTFMH |
| 685 | P HCDR1 | GFEPSDTYIH |
| 686 | P HCDR1 | GFEPSDTYMH |
| 687 | P HCDR1 | GFEPVDTFIH |
| 688 | P HCDR1 | GFEPVDTFMH |
| 689 | P HCDR1 | GFEPVDTYIH |
| 690 | P HCDR1 | GFEPVDTYMH |
| 691 | P HCDR1 | GFEVGDTFIH |
| 692 | P HCDR1 | GFEVGDTFMH |
| 693 | P HCDR1 | GFEVGDTYIH |
| 694 | P HCDR1 | GFEVGDTYMH |
| 695 | P HCDR1 | GFEVQDTFIH |
| 696 | P HCDR1 | GFEVQDTFMH |
| 697 | P HCDR1 | GFEVQDTYIH |
| 698 | P HCDR1 | GFEVQDTYMH |
| 699 | P HCDR1 | GFEVSDTFIH |
| 700 | P HCDR1 | GFEVSDTFMH |
| 701 | P HCDR1 | GFEVSDTYIH |
| 702 | P HCDR1 | GFEVSDTYMH |
| 703 | P HCDR1 | GFEVVDTFIH |
| 704 | P HCDR1 | GFEVVDTFMH |
| 705 | P HCDR1 | GFEVVDTYIH |
| 706 | P HCDR1 | GFEVVDTYMH |
| 707 | P HCDR1 | GFX₁X₂X₃DX₄X₅X₆H |
| | | X₁ = D or E |
| | | X₂ = I, L, P, or V |
| | | X₃ = G, Q, S, or V |
| | | X₄ = A, S, T |
| | | X₅ = F or Y |
| | | X₆ = I, L, or M |
| 708 | PHCDR1 | GFX₁X₂X₃DTX₄X₅H |
| | | X₁ = D, OR E |
| | | X₂ = I, P, OR V |
| | | X₃ = G, Q, S, OR V |
| | | X₄ = F, OR Y |
| | | X₅ = I, OR M |
| 709 | M1 HCDR1 | GFTFSSYW |
| 710 | M2 HCDR1 | GGSFTGFY |
| 711 | M3 HCDR1 | GY(X1)F(X2)(X3)YGIS; X1 = P, S, D, Q, N; X2 = T, R; |
| | | X3 = N, T, Y, H |
| 712 | M3 HCDR1 | GYDFTYYGIS |
| 713 | M4 HCDR1 | SRSYYWG |
| 714 | M5 HCDR1 | TSNMGVV |
| 715 | M6 HCDR1 | LYGMN |
| 716 | M6 HCDR1 | NYGMN |
| 717 | M7 HCDR1 | GYTFTSSWMH |
| 718 | M8 HCDR1 | GYTFTSYDIN |
| 719 | M9 HCDR1 | SYFWS |
| 720 | M10 HCDR1 | GYYWN |
| 721 | M11 HCDR1 | GFTFSTYG |
| 722 | M12 HCDR1 | TYGMS |
| 2 | P HCDR2 | RIDPASGHTKYDPKFQV |
| 3 | P HCDR2 | RIEPASGHIKYDPKFQG |
| 4 | P HCDR2 | RIDPASGHIKYDPKFQG |
| 5 | P HCDR2 | RIEPASGHIKYDPKFQV |
| 723 | P HCDR2 | WIFPGDGRTDYNEKFKG |
| 724 | P HCDR2 | RLDPASGHTK |
| 725 | P HCDR2 | RIEPASGHTK |
| 726 | P HCDR2 | RIDPESGHTK |
| 727 | P HCDR2 | RIDPASGHTK |
| 728 | P HCDR2 | RIDPAGGHTK |
| 729 | P HCDR2 | RIDPASAHTK |
| 730 | P HCDR2 | RIDPASGHIK |
| 731 | P HCDR2 | RIDPASGHLK |
| 732 | P HCDR2 | RIDPASGHVK |
| 733 | P HCDR2 | YDPKFQV |
| 734 | P HCDR2 | IDPKFQV |
| 735 | P HCDR2 | LDPKFQV |
| 736 | P HCDR2 | MDPKFQV |
| 737 | P HCDR2 | SDPKFQV |
| 738 | P HCDR2 | TDPKFQV |
| 739 | P HCDR2 | VDPKFQV |
| 740 | P HCDR2 | YIPKFQV |
| 741 | P HCDR2 | YNPKFQV |
| 742 | P HCDR2 | YRPKFQV |
| 743 | P HCDR2 | YSPKFQV |
| 744 | P HCDR2 | YDPKFRV |
| 745 | P HCDR2 | YDPKFQA |
| 746 | P HCDR2 | YDPKFQD |
| 747 | P HCDR2 | YDPKFQE |
| 748 | P HCDR2 | YDPKFQG |
| 749 | P HCDR2 | YDPKFQH |
| 750 | P HCDR2 | YDPKFQK |
| 751 | P HCDR2 | YDPKFQL |
| 752 | P HCDR2 | YDPKFQM |
| 753 | P HCDR2 | YDPKFQN |
| 754 | P HCDR2 | YDPKFQP |
| 755 | P HCDR2 | YDPKFQR |
| 756 | P HCDR2 | YDPKFQS |
| 757 | P HCDR2 | YDPKFQT |
| 758 | P HCDR2 | RIEPASGHIKYDPKFQG |
| 759 | P HCDR2 | RIEPASGHIKYSPKFQG |
| 760 | P HCDR2 | RIEPASGHVKYSPKFQV |
| 761 | P HCDR2 | RIEPASGHVKYDPKFQT |
| 762 | P HCDR2 | RIDPASGHIKYDPKFQK |
| 763 | P HCDR2 | RIDPASGHVKIDPKFQV |
| 764 | P HCDR2 | RIDPASGHLKYDPKFQV |
| 765 | P HCDR2 | RIDPASGHLKYDPKFQR |
| 766 | P HCDR2 | RIDPASGHLKYDPKFQN |
| 767 | P HCDR2 | RIEPASGHLKYDPKFQE |
| 768 | P HCDR2 | PASGH |
| 769 | P HCDR2 | RIDPASGHTKYDPKFQV |
| 770 | P HCDR2 | RIDPASGHLKYDPKFQG |
| 771 | P HCDR2 | RIDPASGHTKYDPKFQG |
| 772 | P HCDR2 | RIDPASGHSKYDPKFQV |
| 773 | P HCDR2 | RIDPASGHYKYDPKFQV |
| 774 | P HCDR2 | RX₁X₂PX₃X₄X₅HX₆KX₇X₈PKFX₉X₁₀ |
| | | X₁ = I or L |
| | | X₂ = D or E |
| | | X₃ = A or E |
| | | X₄ = G or S |
| | | X₅ = A or G |
| | | X₆ = I, L, T, or V |
| | | X₇ = I, L, M, S, T, V, or Y |
| | | X₈ = D, I, N, R, or S |
| | | X₉ = Q or R |
| | | X₁₀ = A, D, E, G, H, K, L, M, N, P, R, S, T, or V |
| 775 | M1 HCDR2 | IKEDGSEK |
| 776 | M2 HCDR2 | INHRGNT |
| 777 | M3 HCDR2 | WIS(X1)YNG(X2)(X3)(X4)YA(X5)(X6)(X7)QG; X1 = |
| | | T, P, S, A; X2 = N, G, V, K, A; X3 = T, K; X4 = H, |
| | | N; X5 = Q, R; X6 = K, M; X7 = L, H |
| 778 | M3 HCDR2 | WISTYNGNTHYARMLQG |
| 779 | M4 HCDR2 | SIYYNGRTYYNPSLKS |
| 780 | M5 HCDR2 | HILWDDREYSNPALKS |
| 781 | M6 HCDR2 | WINTYTGEPTYADDFKG |
| 782 | M7 HCDR2 | IHPNSGGT |
| 783 | M8 HCDR2 | WLNPNSGXTG; X = N, Y |
| 784 | M9 HCDR2 | YIYYSGNTKYNPSLKS |
| 785 | M10 HCDR2 | EINHAGNTNYNPSLKS |
| 786 | M11 HCDR2 | ISGTGRTT |
| 787 | M12 HCDR2 | WMNTYSGVTTYADDFKG |
| 6 | P HCDR3 | SGGLPDV |
| 7 | P HCDR3 | ARSGGLPDV |
| 8 | P HCDR3 | SGGLPDW |
| 9 | P HCDR3 | ARSGGLPDW |
| 788 | P HCDR3 | YGPAMDY |
| 789 | P HCDR3 | LGGLPDV |
| 790 | P HCDR3 | SAGLPDV |
| 791 | P HCDR3 | SGGAPDV |
| 792 | P HCDR3 | SGGMPDV |
| 793 | P HCDR3 | SGGLPEV |
| 794 | P HCDR3 | SGGLPDK |
| 795 | P HCDR3 | SGGLPDM |
| 796 | P HCDR3 | SGGLPDQ |
| 797 | P HCDR3 | SGGLPDR |
| 798 | P HCDR3 | SGGLPDS |
| 799 | P HCDR3 | SGGLPDT |
| 800 | P HCDR3 | SGGLPDH |
| 801 | P HCDR3 | SGGLPDF |
| 802 | P HCDR3 | SGGSPDV |
| 803 | P HCDR3 | ARSGGLPDM |
| 804 | P HCDR3 | ARSGGLPDK |
| 805 | P HCDR3 | SGGLPD |
| 806 | P HCDR3 | ARSGGLPDF |
| 807 | P HCDR3 | ARSGGLPDL |
| 808 | P HCDR3 | SGGLPDE |
| 809 | P HCDR3 | SGGLPDI |
| 810 | P HCDR3 | SGGLPDK |
| 811 | P HCDR3 | SGGLPDL |
| 812 | P HCDR3 | SGGLPDM |
| 813 | P HCDR3 | SGGLPDQ |
| 814 | P HCDR3 | SGGLPDT |
| 815 | P HCDR3 | SGGLPDW |
| 816 | P HCDR3 | SGGLPDY |
| 817 | P HCDR3 | SGGMPDE |
| 818 | P HCDR3 | SGGMPDI |
| 819 | P HCDR3 | SGGMPDK |
| 820 | P HCDR3 | SGGMPDL |
| 821 | P HCDR3 | SGGMPDM |
| 822 | P HCDR3 | SGGMPDQ |
| 823 | P HCDR3 | SGGMPDT |
| 824 | P HCDR3 | SGGMPDV |
| 825 | P HCDR3 | SGGMPDW |
| 826 | P HCDR3 | SGGMPDY |
| 827 | P HCDR3 | X₁X₂GX₃PX₄X₅ |
| | | X1 = L or S |
| | | X2 = A or G |
| | | X3 = A, L, or M |
| | | X4 = D or E |
| | | X5 = K, M, Q, R, S, T, V, or W |
| 828 | P HCDR3 | SGGX₁PDX₂ |
| | | X₁ = L, OR M |
| | | X₂ = E, I, K, L, M, Q, T, V, W, OR Y |
| 829 | M1 HCDR3 | AREDYDSYYKYGMDV |
| 830 | M2 HCDR3 | ASPFYDFWSGSDY |
| 831 | M3 HCDR3 | ENYYGSG(X1)(X2)RGGMD(X3); X1 = S, A; X2 = Y, |
| | | P; X3 = V, A, G |
| 832 | M3 HCDR3 | ENYYGSGAYRGGMDV |
| 833 | M4 HCDR3 | EDYGDYGAFDI |
| 834 | M5 HCDR3 | MSRNYYGSSYVMDY |
| 835 | M6 HCDR3 | DTAMDYAMAY |
| 836 | M6 HCDR3 | DYGKYGDYYAMDY |
| 837 | M7 HCDR3 | ARGDYYGYVSWFAY |
| 838 | M8 HCDR3 | EVPETAAFEY |
| 839 | M9 HCDR3 | ETGSYYGFDY |
| 840 | M10 HCDR3 | GYCRSTTCYFDY |
| 841 | M11 HCDR3 | TKERGDYYYGVFDY |
| 842 | M12 HCDR3 | EGYVFDDYYATDY |
| 10 | P LCDR1 | RASSSVSYMY |
| 843 | P LCDR1 | RSSQTIVHSNGDTYLD |
| 844 | P LCDR1 | GASSSVSYMY |
| 845 | P LCDR1 | WASSSVSYMY |
| 846 | P LCDR1 | RASSSVIYMY |
| 847 | P LCDR1 | RASSSVSFMY |
| 848 | P LCDR1 | RASSSVSYLY |
| 849 | P LCDR1 | RASSSVSYMR |
| 850 | P LCDR1 | GASSSVSYMY |
| 851 | P LCDR1 | ASSSVSYMY |
| 852 | P LCDR1 | X₁ASSSVX₂X₃X₄X₅ |
| | | X1 = G, R, or W |
| | | X2 = I or S |
| | | X3 = F or Y |
| | | X4 = L or M |
| | | X5 = R or Y |
| 853 | M1 LCDR1 | QSILYSSNNKNY |
| 854 | M2 LCDR1 | QSL VHSDGNTY |
| 855 | M3 LCDR1 | RASQSVSSYLA |
| 856 | M4 LCDR1 | RASQGISSALA |
| 857 | M5 LCDR1 | SASSSVNYMH |
| 858 | M6 LCDR1 | KSSQNIVHSDGNTYLE |
| 859 | M6 LCDR1 | RSSQSIVHSNGNTYLD |
| 860 | M7 LCDR1 | QNINVL |
| 861 | M8 LCDR1 | TSSSSDIGA(X1)(X2)GV(X3); X1 = G, A; X2 = L, S, Q; |
| | | X3 = H, L |
| 862 | M9 LCDR1 | RASQSINNYLN |
| 863 | M10 LCDR1 | RASQSVRSSYLA |
| 864 | M11 LCDR1 | QTISSW |
| 865 | M12 LCDR1 | RSSQNIVHSDGNTYLE |
| 11 | P LCDR2 | ATSNLAS |
| 866 | P LCDR2 | KVSNRFS |
| 867 | P LCDR2 | AKSNLAS |
| 868 | P LCDR2 | ATPNLAS |
| 869 | P LCDR2 | ATENLAS |
| 870 | P LCDR2 | ATSLLAS |
| 871 | P LCDR2 | ATSPLAS |
| 872 | P LCDR2 | ATSNLTS |
| 873 | P LCDR2 | AX₁X₂X₃LX₄S |
| | | X1 = K or T |
| | | X2 = E, P, or S |
| | | X3 = L, N, or P |
| | | X4 = A or T |
| 874 | M1 LCDR2 | WAS |
| 875 | M2 LCDR2 | KIS |
| 876 | M3 LCDR2 | DASNRAT |
| 877 | M4 LCDR2 | DASSLES |
| 878 | M5 LCDR2 | STSNLAS |
| 879 | M6 LCDR2 | KVSNRFS |
| 880 | M7 LCDR2 | KAS |
| 881 | M8 LCDR2 | GYYNRPS |
| 882 | M9 LCDR2 | AASSLQS |
| 883 | M10 LCDR2 | GASSRAT |
| 884 | M11 LCDR2 | AAS |
| 885 | M12 LCDR2 | KVSNRFS |
| 12 | P LCDR3 | QQWEGNPRT |
| 13 | P LCDR3 | QQWKGNPRT |
| 14 | P LCDR3 | QQWSGNPRT |
| 15 | P LCDR3 | QQWSRNPRT |
| 886 | P LCDR3 | FQGSHVPYT |
| 887 | P LCDR3 | HQWSGNPRT |
| 888 | P LCDR3 | NQWSGNPRT |
| 889 | P LCDR3 | SQWSGNPRT |
| 890 | P LCDR3 | QQSSGNPRT |
| 891 | P LCDR3 | QQWDGNPRT |
| 892 | P LCDR3 | QQWHGNPRT |
| 893 | P LCDR3 | QQWNGNPRT |
| 894 | P LCDR3 | QQWQGNPRT |
| 895 | P LCDR3 | QQWVGNPRT |
| 896 | P LCDR3 | QQWSANPRT |
| 897 | P LCDR3 | QQWSDNPRT |
| 898 | P LCDR3 | QQWSQNPRT |
| 899 | P LCDR3 | QQWSSNPRT |
| 900 | P LCDR3 | QQWSGNPRS |
| 901 | P LCDR3 | QQFSGNPRT |
| 902 | P LCDR3 | QQHSGNPRT |
| 903 | P LCDR3 | QQISGNPRT |
| 904 | P LCDR3 | QQPSGNPRT |
| 905 | P LCDR3 | QQRSGNPRT |
| 906 | P LCDR3 | QQYSGNPRT |
| 907 | P LCDR3 | QQWSGHPRT |
| 908 | P LCDR3 | QQWSGLPRT |
| 909 | P LCDR3 | QQWSGQPRT |
| 910 | P LCDR3 | QQWSGSPRT |
| 911 | P LCDR3 | QQWSGTPRT |
| 912 | P LCDR3 | QQWSGMPRT |
| 913 | P LCDR3 | QQWSGFPRT |
| 914 | P LCDR3 | QQWSGKPRT |
| 915 | P LCDR3 | QQWSGRPRT |
| 916 | P LCDR3 | QQWSGDPRT |
| 917 | P LCDR3 | QQWAGNPRT |
| 918 | P LCDR3 | QQWYGNPRT |
| 919 | P LCDR3 | QQWFGNPRT |
| 920 | P LCDR3 | QQWQGNPRT |
| 921 | P LCDR3 | GNPRT |
| 922 | P LCDR3 | SQWSGNPRT |
| 923 | P LCDR3 | QQWSGNPRS |
| 924 | P LCDR3 | QQWSGTPRT |
| 925 | P LCDR3 | QQWSGDPRT |
| 926 | P LCDR3 | QQWSGFPRT |
| 927 | P LCDR3 | QQWSGKPRT |
| 928 | P LCDR3 | QQWSGRPRT |
| 929 | P LCDR3 | QQWSGSPRT |
| 930 | P LCDR3 | QQWDADPRT |
| 931 | P LCDR3 | QQWDAFPRT |
| 932 | P LCDR3 | QQWDAKPRT |
| 933 | P LCDR3 | QQWDANPRT |
| 934 | P LCDR3 | QQWDARPRT |
| 935 | P LCDR3 | QQWDASPRT |
| 936 | P LCDR3 | QQWDATPRT |
| 937 | P LCDR3 | QQWDGDPRT |
| 938 | PLCDR3 | QQWDGFPRT |
| 939 | P LCDR3 | QQWDGKPRT |
| 940 | P LCDR3 | QQWDGNPRT |
| 941 | P LCDR3 | QQWDGRPRT |
| 942 | P LCDR3 | QQWDGSPRT |
| 943 | P LCDR3 | QQWDGTPRT |
| 944 | P LCDR3 | QQWEADPRT |
| 945 | P LCDR3 | QQWEAFPRT |
| 946 | P LCDR3 | QQWEAKPRT |
| 947 | P LCDR3 | QQWEANPRT |
| 948 | P LCDR3 | QQWEARPRT |
| 949 | P LCDR3 | QQWEASPRT |
| 950 | P LCDR3 | QQWEATPRT |
| 951 | P LCDR3 | QQWEGDPRT |
| 952 | P LCDR3 | QQWEGFPRT |
| 953 | P LCDR3 | QQWEGKPRT |
| 954 | P LCDR3 | QQWEGRPRT |
| 955 | P LCDR3 | QQWEGSPRT |
| 956 | P LCDR3 | QQWEGTPRT |
| 957 | P LCDR3 | QQWHADPRT |
| 958 | P LCDR3 | QQWHAFPRT |
| 959 | P LCDR3 | QQWHAKPRT |
| 960 | P LCDR3 | QQWHANPRT |
| 961 | P LCDR3 | QQWHARPRT |
| 962 | P LCDR3 | QQWHASPRT |
| 963 | P LCDR3 | QQWHATPRT |
| 964 | P LCDR3 | QQWHGDPRT |
| 965 | P LCDR3 | QQWHGFPRT |
| 966 | P LCDR3 | QQWHGKPRT |
| 967 | P LCDR3 | QQWHGNPRT |
| 968 | P LCDR3 | QQWHGRPRT |
| 969 | P LCDR3 | QQWHGSPRT |
| 970 | P LCDR3 | QQWHGTPRT |
| 971 | P LCDR3 | QQWNADPRT |
| 972 | P LCDR3 | QQWNAFPRT |
| 973 | P LCDR3 | QQWNAKPRT |
| 974 | P LCDR3 | QQWNANPRT |
| 975 | P LCDR3 | QQWNARPRT |
| 976 | P LCDR3 | QQWNASPRT |
| 977 | P LCDR3 | QQWNATPRT |
| 978 | P LCDR3 | QQWNGDPRT |
| 979 | P LCDR3 | QQWNGFPRT |
| 980 | P LCDR3 | QQWNGKPRT |
| 981 | P LCDR3 | QQWNGNPRT |
| 982 | P LCDR3 | QQWNGRPRT |
| 983 | P LCDR3 | QQWNGSPRT |
| 984 | P LCDR3 | QQWNGTPRT |
| 985 | P LCDR3 | QQWQADPRT |
| 986 | P LCDR3 | QQWQAFPRT |
| 987 | P LCDR3 | QQWQAKPRT |
| 988 | P LCDR3 | QQWQANPRT |
| 989 | P LCDR3 | QQWQARPRT |
| 990 | P LCDR3 | QQWQASPRT |
| 991 | P LCDR3 | QQWQATPRT |
| 992 | P LCDR3 | QQWQGDPRT |
| 993 | P LCDR3 | QQWQGFPRT |
| 994 | P LCDR3 | QQWQGKPRT |
| 995 | P LCDR3 | QQWQGRPRT |
| 996 | P LCDR3 | QQWQGSPRT |
| 997 | P LCDR3 | QQWQGTPRT |
| 998 | P LCDR3 | QQWSADPRT |
| 999 | P LCDR3 | QQWSAFPRT |
| 1000 | P LCDR3 | QQWSAKPRT |
| 1001 | P LCDR3 | QQWSANPRT |
| 1002 | P LCDR3 | QQWSARPRT |
| 1003 | P LCDR3 | QQWSASPRT |
| 1004 | P LCDR3 | QQWSATPRT |
| 1005 | P LCDR3 | NQWDAFPRT |
| 1006 | P LCDR3 | NQWDAKPRT |
| 1007 | P LCDR3 | NQWDANPRT |
| 1008 | P LCDR3 | NQWDARPRT |
| 1009 | P LCDR3 | NQWDASPRT |
| 1010 | P LCDR3 | NQWDATPRT |
| 1011 | P LCDR3 | NQWDGDPRT |
| 1012 | P LCDR3 | NQWDGFPRT |
| 1013 | P LCDR3 | NQWDGKPRT |
| 1014 | P LCDR3 | NQWDGNPRT |
| 1015 | P LCDR3 | NQWDGRPRT |
| 1016 | P LCDR3 | NQWDGSPRT |
| 1017 | P LCDR3 | NQWDGTPRT |
| 1018 | P LCDR3 | NQWEADPRT |
| 1019 | P LCDR3 | NQWEAFPRT |
| 1020 | P LCDR3 | NQWEAKPRT |
| 1021 | P LCDR3 | NQWEANPRT |
| 1022 | P LCDR3 | NQWEARPRT |
| 1023 | P LCDR3 | NQWEASPRT |
| 1024 | P LCDR3 | NQWEATPRT |
| 1025 | P LCDR3 | NQWEGDPRT |
| 1026 | P LCDR3 | NQWEGFPRT |
| 1027 | P LCDR3 | NQWEGKPRT |
| 1028 | P LCDR3 | NQWEGNPRT |
| 1029 | P LCDR3 | NQWEGRPRT |
| 1030 | P LCDR3 | NQWEGSPRT |
| 1031 | P LCDR3 | NQWEGTPRT |
| 1032 | P LCDR3 | NQWHADPRT |
| 1033 | P LCDR3 | NQWHAFPRT |
| 1034 | P LCDR3 | NQWHAKPRT |
| 1035 | P LCDR3 | NQWHANPRT |
| 1036 | P LCDR3 | NQWHARPRT |
| 1037 | P LCDR3 | NQWHASPRT |
| 1038 | P LCDR3 | NQWHATPRT |
| 1039 | P LCDR3 | NQWHGDPRT |
| 1040 | P LCDR3 | NQWHGFPRT |
| 1041 | P LCDR3 | NQWHGKPRT |
| 1042 | P LCDR3 | NQWHGNPRT |
| 1043 | P LCDR3 | NQWHGRPRT |
| 1044 | P LCDR3 | NQWHGSPRT |
| 1045 | P LCDR3 | NQWHGTPRT |
| 1046 | P LCDR3 | NQWNADPRT |
| 1047 | P LCDR3 | NQWNAFPRT |
| 1048 | P LCDR3 | NQWNAKPRT |
| 1049 | P LCDR3 | NQWNANPRT |
| 1050 | P LCDR3 | NQWNARPRT |
| 1051 | P LCDR3 | NQWNASPRT |
| 1052 | P LCDR3 | NQWNATPRT |
| 1053 | P LCDR3 | NQWNGDPRT |
| 1054 | P LCDR3 | NQWNGFPRT |
| 1055 | P LCDR3 | NQWNGKPRT |
| 1056 | P LCDR3 | NQWNGNPRT |
| 1057 | P LCDR3 | NQWNGRPRT |
| 1058 | P LCDR3 | NQWNGSPRT |
| 1059 | P LCDR3 | NQWNGTPRT |
| 1060 | P LCDR3 | NQWQADPRT |
| 1061 | P LCDR3 | NQWQAFPRT |
| 1062 | P LCDR3 | NQWQAKPRT |
| 1063 | P LCDR3 | NQWQANPRT |
| 1064 | P LCDR3 | NQWQARPRT |
| 1065 | P LCDR3 | NQWQASPRT |
| 1066 | P LCDR3 | NQWQATPRT |
| 1067 | P LCDR3 | NQWQGDPRT |
| 1068 | P LCDR3 | NQWQGFPRT |
| 1069 | P LCDR3 | NQWQGKPRT |
| 1070 | P LCDR3 | NQWQGNPRT |
| 1071 | P LCDR3 | NQWQGRPRT |
| 1072 | P LCDR3 | NQWQGSPRT |
| 1073 | P LCDR3 | NQWQGTPRT |
| 1074 | P LCDR3 | NQWSADPRT |
| 1075 | PLCDR3 | NQWSAFPRT |
| 1076 | P LCDR3 | NQWSAKPRT |
| 1077 | P LCDR3 | NQWSANPRT |
| 1078 | P LCDR3 | NQWSARPRT |
| 1079 | P LCDR3 | NQWSASPRT |
| 1080 | PLCDR3 | NQWSATPRT |
| 1081 | PLCDR3 | NQWSGDPRT |
| 1082 | P LCDR3 | NQWSGFPRT |
| 1083 | PLCDR3 | NQWSGKPRT |
| 1084 | P LCDR3 | NQWSGNPRT |
| 1085 | PLCDR3 | NQWSGRPRT |
| 1086 | PLCDR3 | NQWSGSPRT |
| 1087 | PLCDR3 | NQWSGTPRT |
| 1088 | PLCDR3 | X₁QWX₂X₃X₄PRT |
| | | X₁ = Q, OR N |
| | | X₂ = D, E, H, N, Q, OR S |
| | | X₃ = A, OR G |
| | | X₄ = D, F, K, N, R, S, OR T |
| 1089 | P LCDR3 | X₁QX₂X₃X₄X₅PRX₆ |
| | | X1 = H, N, Q, or S |
| | | X2 = F, H, I, P, R, S, W, or Y |
| | | X3 = D, E, H, N, Q, S, or V |
| | | X4 = A, D, G, Q, or S |
| | | X5 = D, F, H, K, L, M, N, Q, R, S, or T |
| | | X6 = T or S |
| 1090 | M1 LCDR3 | QQYYSTPFT |
| 1091 | M2 LCDR3 | MQATQFPLT |
| 1092 | M3 LCDR3 | QQRSNWPWT |
| 1093 | M4 LCDR3 | QQFNSYPLT |
| 1094 | M5 LCDR3 | HQWNNYGT |
| 1095 | M6 LCDR3 | FQGSHVPLT |
| 1096 | M7 LCDR3 | QQGQSYPYT |
| 1097 | M8 LCDR3 | QSXDGTLSAL; X = Y, W, F |
| 1098 | M9 LCDR3 | QQSYSTPRT |
| 1099 | M10 LCDR3 | QQYGSSPT |
| 1100 | M11 LCDR3 | QQYHRSWT |
| 1101 | M12 LCDR3 | FQGSHVPLT |

TABLE 16
Heavy Chain Variable Region (VH) Amino Acid Sequences

| SEQ | | |
|---|---|---|
| ID | | |
| NO | Description | Sequence |
| 101 | 217 VH, 158 VH | QVQLVQSGAEVKKPGASVKVSCKASGFDIQDTYMHW |
| | | VKQRPGQGLEWMGRIDPASGHTKYDPKFQVRVTITRD |
| | | TSTSTVYMELSSLRSEDTAVYYCARSGGLPDVWGQGT |
| | | TVTVSS |
| 102 | 220 VH, 160 VH | QVQLVQSGAEVKKPGASVKVSCKASGFDIQDTYMHW |
| | | VKQRPGQGLEWMGRIDPASGHTKYDPKFQVRVTMTR |
| | | DTSTSTAYLELSSLRSEDTAVYYCARSGGLPDVWGQG |
| | | TTVTVSS |
| 103 | 223 VH, 200 VH, | EVQLVQSGAEVKKPGASVKVSCKASGFDIQDTYMHW |
| | 194 VL, 206 VH | VRQRPGQGLEWIGRIDPASGHTKYDPKFQVRATITTDT |
| | | STSTAYLELSSLRSEDTAVYYCARSGGLPDVWGQGTT |
| | | VTVSS |
| 104 | 219 VH, 157 VH | QVQLVQSGAEVKKPGASVKVSCKASGFDIQDTYMHW |
| | | VKQRPGQGLEWMGRIDPASGHTKYDPKFQVRVTITRD |
| | | TSTSTVYLELSSLRSEDTAVYYCARSGGLPDVWGQGT |
| | | TVTVSS |
| 105 | 221 VH, 125 VH | QVQLVQSGAEVKKPGASVKVSCKASGFDIQDTYMHW |
| | | VKQRPGQGLEWMGRIDPASGHTKYDPKFQVRATITRD |
| | | TSTSTAYLELSSLRSEDTAVYYCARSGGLPDVWGQGT |
| | | TVTVSS |
| 106 | 213 VH, 162 VH | QVQLVQSGAEVKKPGASVKVSCKASGFDIQDTYMHW |
| | | VRQRPGQGLEWMGRIDPASGHTKYDPKFQVRVTITTD |
| | | TSTSTVYMELSSLRSEDTAVYYCARSGGLPDVWGQGT |
| | | TVTVSS |
| 107 | 212 VH, 100 VH, | QVQLVQSGAEVKKPGASVKVSCKASGFDIQDTYMHW |
| | 181 VH, 34 VH, 79 | VRQRPGQGLEWIGRIDPASGHTKYDPKFQVRATITTDT |
| | VH | STSTAYLELSSLRSEDTAVYYCARSGGLPDVWGQGTT |
| | | VTVSS |
| 108 | 107 VH, 211 VH, | QVQLVQSGAEVKKPGASVKVSCKASGFDIQDTYMHW |
| | 15 VH, 30 VH, 29 | VKQRPGQGLEWIGRIDPASGHTKYDPKFQVRATITTDT |
| | VH, 48 VH, 49 VH, | STSTAYLELSSLRSEDTAVYYCARSGGLPDVWGQGTT |
| | 50 VH, 51 VH, 52 | VTVSS |
| | VH, 56 VH | |
| 109 | 205 VH, 199 VH, | EVQLVQSGAEVKKPGASVKVSCKASGFDIQDTYMHW |
| | 55 VH, 193 VH | VKQRPGQGLEWIGRIDPASGHTKYDPKFQVRATITTDT |
| | | STSTAYLELSSLRSEDTAVYYCARSGGLPDVWGQGTT |
| | | VTVSS |
| 110 | 129 VH, 139 VH, | QVQLVQSGAEVKKPGASVKVSCKASGFDIQDTYMHW |
| | 140 VH, 215 VH | VRQRPGQGLEWMGRIDPASGHTKYDPKFQVRVTITTD |
| | | TSTSTAYLELSSLRSEDTAVYYCARSGGLPDVWGQGT |
| | | TVTVSS |
| 111 | 214 VH, 128 VH, | QVQLVQSGAEVKKPGASVKVSCKASGFDIQDTYMHW |
| | 141 VH, 142 VH, | VRQRPGQGLEWMGRIDPASGHTKYDPKFQVRVTITRD |
| | 144 VH | TSTSTAYLELSSLRSEDTAVYYCARSGGLPDVWGQGT |
| | | TVTVSS |
| 112 | 216 VH, 123 VH | QVQLVQSGAEVKKPGASVKVSCKASGFDIQDTYMHW |
| | | VRQRPGQGLEWIGRIDPASGHTKYDPKFQVRVTITRDT |
| | | STSTAYLELSSLRSEDTAVYYCARSGGLPDVWGQGTT |
| | | VTVSS |
| 113 | 122 VH | QVQLVQSGAEVKKPGASVKVSCKASGFDIQDTYMHW |
| | | VRQRPGQGLEWIGRIDPASGHTKYDPKFQVRATITRDT |
| | | STSTAYLELSSLRSEDTAVYYCARSGGLPDVWGQGTT |
| | | VTVSS |
| 114 | 222 VH, 126 VH | QVQLVQSGAEVKKPGASVKVSCKASGFDIQDTYMHW |
| | | VKQRPGQGLEWMGRIDPASGHTKYDPKFQVRVTITRD |
| | | TSTSTAYLELSSLRSEDTAVYYCARSGGLPDVWGQGT |
| | | TVTVSS |
| 115 | 188 VH, 41 VH, | QVQLVQSGAEVKKPGASVKVSCKASGFDIQDTYMHW |
| | 102 VH | VKQRPGQGLEWIGRIDPASGHTKYDPKFQVRVTITTDT |
| | | STSTAYLELSSLRSEDTAVYYCARSGGLPDVWGQGTT |
| | | VTVSS |
| 116 | 203 VH, 197 VH, | EVQLVQSGAEVKKPGASVKVSCKASGFDIQDTYMHW |
| | 209 VH, 224 VH | VKQAPGQGLEWMGRIEPASGHIKYDPKFQGRVTMTR |
| | | DTSTSTVYMELSSLRSEDTAVYYCARSGGLPDWWGQ |
| | | GTTVTVSS |
| 117 | 147 VH, 112 VH, | QVQLVQSGAEVKKPGASVKVSCKASGFDIQDTYMHW |
| | 59 VH | VKQRPGQGLEWMGRIEPASGHIKYDPKFQGRVTMTR |
| | | DTSTSTVYMELSSLRSEDTAVYYCARSGGLPDWWGQ |
| | | GTTVTVSS |
| 118 | 127 VH | QVQLVQSGAEVKKPGASVKVSCKASGFDIQDTYMHW |
| | | VKQRPGQGLEWMGRIDPASGHTKYDPKFQVRVTITTD |
| | | TSTSTAYLELSSLRSEDTAVYYCARSGGLPDVWGQGT |
| | | TVTVSS |
| 119 | 159 VH, 218 VH | QVQLVQSGAEVKKPGASVKVSCKASGFDIQDTYMHW |
| | | VKQRPGQGLEWMGRIDPASGHTKYDPKFQVRVTITRD |
| | | TSTSTAYMELSSLRSEDTAVYYCARSGGLPDVWGQGT |
| | | TVTVSS |
| 120 | 103 VH, 45 VH, | QVQLVQSGAEVKKPGASVKVSCKASGFDIQDTYMHW |
| | 167 VH, 187 VH | VKQRPGQGLEWIGRIDPASGHTKYDPKFQVRVTITRDT |
| | | STSTAYLELSSLRSEDTAVYYCARSGGLPDVWGQGTT |
| | | VTVSS |
| 121 | 64 VH, 148 VH, 97 | QVQLVQSGAEVKKPGASVKVSCKASGFDIQDTYMHW |
| | VH, 114 VH, 130 | VRQAPGQGLEWMGRIEPASGHIKYDPKFQVRVTMTR |
| | VH, 133 VH, 137 | DTSTSTVYMELSSLRSEDTAVYYCARSGGLPDWWGQ |
| | VH, 155 VH | GTTVTVSS |
| 122 | 67 VH, 138 VH, | QVQLVQSGAEVKKPGASVKVSCKASGFDIQDTYMHW |
| | 115 VH, 149 VH, | VRQAPGQGLEWMGRIEPASGHIKYDPKFQVRATMTR |
| | 134 VH, 98 VH, | DTSTSTVYMELSSLRSEDTAVYYCARSGGLPDWWGQ |
| | 156 VH | GTTVTVSS |
| 123 | 68 VH, 99 VH, 116 | QVQLVQSGAEVKKPGASVKVSCKASGFDIQDTYMHW |
| | VH | VRQAPGQGLEWMGRIEPASGHIKYDPKFQVRVTITRD |
| | | TSTSTVYMELSSLRSEDTAVYYCARSGGLPDWWGQG |
| | | TTVTVSS |
| 124 | 94 VH, 113 VH, | QVQLVQSGAEVKKPGASVKVSCKASGFDIQDTYMHW |
| | 151 VH, 78 VH | VRQAPGQGLEWMGRIEPASGHIKYDPKFQVRATITRD |
| | | TSTSTVYMELSSLRSEDTAVYYCARSGGLPDWWGQG |
| | | TTVTVSS |
| 125 | 110 VH, 58 VH, | QVQLVQSGAEVKKPGASVKVSCKASGFDIQDTYMHW |
| | 136 VH, 146 VH, | VKQAPGQGLEWMGRIEPASGHIKYDPKFQGR VTMTR |
| | 154 VH | DTSTSTVYMELSSLRSEDTAVYYCARSGGLPDWWGQ |
| | | GTTVTVSS |
| 126 | 169 VH, 175 VH | QVQLVQSGAEVKKPGASVKVSCKASGFDIQDTYMHW |
| | | VKQAPGQGLEWMGRIDPASGHIKYDPKFQGRVTMTR |
| | | DTSTSTVYMELSSLRSEDTAVYYCARSGGLPDWWGQ |
| | | GTTVTVSS |
| 127 | 173 VH, 179 VH | QVQLVQSGAEVKKPGASVKVSCKASGFDIQDTYMHW |
| | | VKQAPGQGLEWMGRIEPASGHIKYDPKFQGRATMTR |
| | | DTSTSTVYMELSSLRSEDTAVYYCARSGGLPDWWGQ |
| | | GTTVTVSS |
| 128 | 96 VH, 132 VH, 65 | QVQLVQSGAEVKKPGASVKVSCKASGFDIQDTYMHW |
| | VH, 150 VH | VRQAPGQGLEWMGRIEPASGHIKYDPKFQGRATMTR |
| | | DTSTSTVYMELSSLRSEDTAVYYCARSGGLPDWWGQ |
| | | GTTVTVSS |
| 129 | 196 VH, 202 VH, | EVQLVQSGAEVKKPGASVKVSCKASGFDIQDTYMHW |
| | 208 VH | VRQAPGQGLEWMGRIEPASGHIKYDPKFQGRATMTR |
| | | DTSTSTVYMELSSLRSEDTAVYYCARSGGLPDWWGQ |
| | | GTTVTVSS |
| 130 | 172 VH, 178 VH | QVQLVQSGAEVKKPGASVKVSCKASGFDIQDTYMHW |
| | | VRQAPGQGLEWMGRIDPASGHIKYDPKFQGRATMTR |
| | | DTSTSTVYMELSSLRSEDTAVYYCARSGGLPDWWGQ |
| | | GTTVTVSS |
| 131 | 75 VH, 72 VH, 95 | QVQLVQSGAEVKKPGASVKVSCKASGFDIQDTYMHW |
| | VH, 152 VH | VRQAPGQGLEWMGRIEPASGHIKYDPKFQGRATITTD |
| | | TSTSTVYMELSSLRSEDTAVYYCARSGGLPDWWGQG |
| | | TTVTVSS |
| 132 | 174 VH, 180 VH | QVQLVQSGAEVKKPGASVKVSCKASGFDIQDTYMHW |
| | | VRQAPGQGLEWMGRIEPASGHIKYDPKFQGRATMTR |
| | | DTSTSTAYMELSSLRSEDTAVYYCARSGGLPDWWGQ |
| | | GTTVTVSS |
| 133 | 109 VH, 91 VH, | QVQLVQSGAEVKKPGASVKVSCKASGFDIQDTYMHW |
| | 135 VH, 145 VH, | VRQAPGQGLEWMGRIEPASGHIKYDPKFQGRVTMTR |
| | 153 VH | DTSTSTAYMELSSLRSEDTAVYYCARSGGLPDWWGQ |
| | | GTTVTVSS |
| 134 | 198 VH, 204 VH, | EVQLVQSGAEVKKPGASVKVSCKASGFDIQDTYMHW |
| | 210 VH | VRQAPGQGLEWMGRIEPASGHIKYDPKFQGRVTMTR |
| | | DTSTSTAYMELSSLRSEDTAVYYCARSGGLPDWWGQ |
| | | GTTVTVSS |
| 135 | 170 VH, 176 VH | QVQLVQSGAEVKKPGASVKVSCKASGFDIQDTYMHW |
| | | VRQAPGQGLEWMGRIDPASGHIKYDPKFQGRVTMTR |
| | | DTSTSTAYMELSSLRSEDTAVYYCARSGGLPDWWGQ |
| | | GTTVTVSS |
| 136 | 31 VH, 85 VH, 86 | QVQLVQSGAEVKKPGASVKVSCKASGFDIQDTYMHW |
| | VH, 87 VH, 88 VH, | VRQAPGQGLEWMGRIEPASGHIKYDPKFQGRVTMTR |
| | 89 VH, 90 VH, 143 | DTSTSTVYMELSSLRSEDTAVYYCARSGGLPDWWGQ |
| | VH, clone 34 VH | GTTVTVSS |
| 137 | 32 VH, 33 VH | DVQLVQSGAEVKKPGASVKVSCKASGFDIQDTYMHW |
| | | VRQAPGQGLEWMGRIEPASGHIKYDPKFQGRVTMTR |
| | | DTSTSTVYMELSSLRSEDTAVYYCARSGGLPDWWGQ |
| | | GTTVTVSS |
| 138 | 35 VH, 182 VH | QVQLVQSGAEVKKPGASVKVSCKASGFDIQDTYMHW |
| | | VKQAPGQGLEWIGRIDPASGHTKYDPKFQVRATITTDT |
| | | STSTAYLELSSLRSEDTAVYYCARSGGLPDVWGQGTT |
| | | VTVSS |
| 139 | 36 VH, 81 VH, 104 | QVQLVQSGAEVKKPGASVKVSCKASGFDIQDTYMHW |
| | VH, 165 VH, | VRQAPGQGLEWIGRIDPASGHTKYDPKFQVRATITTDT |
| | | STSTAYLELSSLRSEDTAVYYCARSGGLPDVWGQGTT |
| | | VTVSS |
| 140 | 37 VH, 82 VH, 101 | QVQLVQSGAEVKKPGASVKVSCKASGFDIQDTYMHW |
| | VH, 183 VH | VKQRPGQGLEWMGRIDPASGHTKYDPKFQVRATITTD |
| | | TSTSTAYLELSSLRSEDTAVYYCARSGGLPDVWGQGT |
| | | TVTVSS |
| 141 | 38 VH, 190 VH | QVQLVQSGAEVKKPGASVKVSCKASGFDIQDTYMHW |
| | | VKQRPGQGLEWIGRIDPASGHTKYDPKFQVRATITTDT |
| | | STSTVYLELSSLRSEDTAVYYCARSGGLPDVWGQGTT |
| | | VTVSS |
| 142 | 39 VH, 191 VH | QVQLVQSGAEVKKPGASVKVSCKASGFDIQDTYMHW |
| | | VKQRPGQGLEWIGRIDPASGHTKYDPKFQVRATITTDT |
| | | STSTAYMELSSLRSEDTAVYYCARSGGLPDVWGQGTT |
| | | VTVSS |
| 143 | 40 VH, 105 VH, | QVQLVQSGAEVKKPGASVKVSCKASGFDIQDTYMHW |
| | 192 VH | VKQRPGQGLEWIGRIDPASGHTKYDPKFQVRATITTDT |
| | | STSTVYMELSSLRSEDTAVYYCARSGGLPDVWGQGTT |
| | | VTVSS |
| 144 | 42 VH, 83 VH, 186 | QVQLVQSGAEVKKPGASVKVSCKASGFDIQDTYMHW |
| | VH | VKQRPGQGLEWIGRIDPASGHTKYDPKFQVRATMTTD |
| | | TSTSTAYLELSSLRSEDTAVYYCARSGGLPDVWGQGT |
| | | TVTVSS |
| 145 | 43 VH, 184 VH | QVQLVQSGAEVKKPGASVKVSCKASGFDIQDTYMHW |
| | | VKQRPGQGLEWIGRIDPASGHTKYDPKFQVRATITRDT |
| | | STSTAYLELSSLRSEDTAVYYCARSGGLPDVWGQGTT |
| | | VTVSS |
| 146 | 44 VH, 53 VH, 166 | QVQLVQSGAEVKKPGASVKVSCKASGFDIQDTYMHW |
| | VH, 189 VH | VKQRPGQGLEWIGRIDPASGHTKYDPKFQVRVTMTTD |
| | | TSTSTAYLELSSLRSEDTAVYYCARSGGLPDVWGQGT |
| | | TVTVSS |
| 147 | 46 VH, 168 VH, | QVQLVQSGAEVKKPGASVKVSCKASGFDIQDTYMHW |
| | 185 VH | VKQRPGQGLEWIGRIDPASGHTKYDPKFQVRATMTRD |
| | | TSTSTAYLELSSLRSEDTAVYYCARSGGLPDVWGQGT |
| | | TVTVSS |
| 148 | 47 VH | QVQLVQSGAEVKKPGASVKVSCKASGFDIQDTYMHW |
| | | VKQRPGQGLEWIGRIDPASGHTKYDPKFQVRVTMTRD |
| | | TSTSTAYLELSSLRSEDTAVYYCARSGGLPDVWGQGT |
| | | TVTVSS |
| 149 | 54 VH | DVQLVQSGAEVKKPGASVKVSCKASGFDIQDTYMHW |
| | | VKQRPGQGLEWIGRIDPASGHTKYDPKFQVRATITTDT |
| | | STSTAYLELSSLRSEDTAVYYCARSGGLPDVWGQGTT |
| | | VTVSS |
| 150 | 57 VH, 111 VH | QVQLVQSGAEVKKPGASVKVSCKASGFDIQDTYMHW |
| | | VRQRPGQGLEWMGRIEPASGHIKYDPKFQGRVTMTRD |
| | | TSTSTVYMELSSLRSEDTAVYYCARSGGLPDWWGQG |
| | | TTVTVSS |
| 151 | 60 VH, 117 VH | QVQLVQSGAEVKKPGASVKVSCKASGFDIQDTYMHW |
| | | VRQAPGQGLEWMGRIDPASGHIKYDPKFQGRVTMTR |
| | | DTSTSTVYMELSSLRSEDTAVYYCARSGGLPDWWGQ |
| | | GTTVTVSS |
| 152 | 61 VH | QVQLVQSGAEVKKPGASVKVSCKASGFDIQDTYMHW |
| | | VRQAPGQGLEWIGRIEPASGHIKYDPKFQGRVTMTRD |
| | | TSTSTVYMELSSLRSEDTAVYYCARSGGLPDWWGQG |
| | | TTVTVSS |
| 153 | 62 VH, 118 VH | QVQLVQSGAEVKKPGASVKVSCKASGFDIQDTYMHW |
| | | VRQAPGQGLEWIGRIDPASGHIKYDPKFQGRVTMTRD |
| | | TSTSTVYMELSSLRSEDTAVYYCARSGGLPDWWGQG |
| | | TTVTVSS |
| 154 | 63 VH, 120 VH | QVQLVQSGAEVKKPGASVKVSCKASGFDIQDTYMHW |
| | | VRQAPGQGLEWMGRIEPASGHVKYDPKFQGRVTMTR |
| | | DTSTSTVYMELSSLRSEDTAVYYCARSGGLPDWWGQ |
| | | GTTVTVSS |
| 155 | 66 VH | QVQLVQSGAEVKKPGASVKVSCKASGFDIQDTYMHW |
| | | VRQAPGQGLEWMGRIEPASGHIKYDPKFQGRVTITRD |
| | | TSTSTVYMELSSLRSEDTAVYYCARSGGLPDWWGQG |
| | | TTVTVSS |
| 156 | 69 VH, 108 VH | EVQLVQSGAEVKKPGASVKVSCKASGFDIQDTYMHW |
| | | VRQAPGQGLEWMGRIEPASGHIKYDPKFQGRVTMTR |
| | | DTSTSTVYMELSSLRSEDTAVYYCARSGGLPDWWGQ |
| | | GTTVTVSS |
| 157 | 70 VH, 73 VH | QVQLVQSGAEVKKPGASVKVSCKASGFDIQDTYMHW |
| | | VRQAPGQGLEWMGRIEPASGHIKYDPKFQGRVTMTTD |
| | | TSTSTVYMELSSLRSEDTAVYYCARSGGLPDWWGQG |
| | | TTVTVSS |
| 158 | 71 VH, 74 VH | QVQLVQSGAEVKKPGASVKVSCKASGFDIQDTYMHW |
| | | VRQAPGQGLEWMGRIEPASGHIKYDPKFQGRVTITTD |
| | | TSTSTVYMELSSLRSEDTAVYYCARSGGLPDWWGQG |
| | | TTVTVSS |
| 159 | 76 VH, 119 VH | QVQLVQSGAEVKKPGASVKVSCKASGFDIQDTYMHW |
| | | VRQAPGQGLEWMGRIEPASGHTKYDPKFQGRVTMTR |
| | | DTSTSTVYMELSSLRSEDTAVYYCARSGGLPDWWGQ |
| | | GTTVTVSS |
| 160 | 77 VH | QVQLVQSGAEVKKPGASVKVSCKASGFDIQDTYMHW |
| | | VRQAPGQGLEWMGRIEPASGHIKYDPKFQGRATITRD |
| | | TSTSTVYMELSSLRSEDTAVYYCARSGGLPDWWGQG |
| | | TTVTVSS |
| 161 | 92 VH | QVQLVQSGAEVKKPGASVKVSCKASGFDIQDTYMHW |
| | | VRQAPGQGLEWMGRIEPASGHIKYDPKFQGRVTMTR |
| | | DTSTSTVYLELSSLRSEDTAVYYCARSGGLPDWWGQG |
| | | TTVTVSS |
| 162 | 93 VH | QVQLVQSGAEVKKPGASVKVSCKASGFDIQDTYMHW |
| | | VRQAPGQGLEWMGRIEPASGHIKYDPKFQGRVTMTR |
| | | DTSTSTAYLELSSLRSEDTAVYYCARSGGLPDWWGQG |
| | | TTVTVSS |
| 163 | 121 VH | QVQLVQSGAEVKKPGASVKVSCKASGFDIQDTYMHW |
| | | VRQRPGQGLEWMGRIDPASGHTKYDPKFQVRATITTD |
| | | TSTSTAYLELSSLRSEDTAVYYCARSGGLPDVWGQGT |
| | | TVTVSS |
| 164 | 124 VH | QVQLVQSGAEVKKPGASVKVSCKASGFDIQDTYMHW |
| | | VRQRPGQGLEWIGRIDPASGHTKYDPKFQVRVTITTDT |
| | | STSTAYLELSSLRSEDTAVYYCARSGGLPDVWGQGTT |
| | | VTVSS |
| 165 | 161 VH | QVQLVQSGAEVKKPGASVKVSCKASGFDIQDTYMHW |
| | | VRQRPGQGLEWMGRIDPASGHTKYDPKFQVRVTITTD |
| | | TSTSTVYLELSSLRSEDTAVYYCARSGGLPDVWGQGT |
| | | TVTVSS |
| 166 | 163 VH | QVQLVQSGAEVKKPGASVKVSCKASGFDIQDTYMHW |
| | | VRQRPGQGLEWMGRIDPASGHTKYDPKFQVRVTITTD |
| | | TSTSTAYMELSSLRSEDTAVYYCARSGGLPDVWGQGT |
| | | TVTVSS |
| 167 | 164 VH | QVQLVQSGAEVKKPGASVKVSCKASGFDIQDTYMHW |
| | | VRQRPGQGLEWMGRIDPASGHTKYDPKFQVRVTMTT |
| | | DTSTSTAYLELSSLRSEDTAVYYCARSGGLPDVWGQG |
| | | TTVTVSS |
| 168 | 171 VH, 177 VH | QVQLVQSGAEVKKPGASVKVSCKASGFDIQDTYMHW |
| | | VRQAPGQGLEWMGRIDPASGHIKYDPKFQGRATITTD |
| | | TSTSTVYMELSSLRSEDTAVYYCARSGGLPDWWGQG |
| | | TTVTVSS |
| 169 | 195 VH, 201 VH, | EVQLVQSGAEVKKPGASVKVSCKASGFDIQDTYMHW |
| | 207 VH | VRQAPGQGLEWMGRIEPASGHIKYDPKFQGRATITTD |
| | | TSTSTVYMELSSLRSEDTAVYYCARSGGLPDWWGQG |
| | | TTVTVSS |
| 1200 | 5C3D11 VH | EVQLQQSGAELVKPGASVKLSCTASGFDIQDTYMHW |
| | | VKQRPEQGLEWIGRIDPASGHTKYDPKFQVKATITTDT |
| | | SSNTAYLQLSSLTSEDTAVYYCSRSGGLPDVWGAGTT |
| | | VTVSS |
| 1201 | 9E12E5 VH | QVHLQQSGPELVKPGASVKLSCKASGYTFTKYDINWV |
| | | RQRPEQGLEWIGWIFPGDGRTDYNEKFKGKATLTTDK |
| | | SSSTAYMEVSRLTSEDSAVYFCARYGPAMDYWGQGT |
| | | SVTVAS |
| 1202 | AS12824 VH | QVQLVQSGAEVKKPGASVKVSCKASGFDICDTYMHW |
| | | VKQRPGQGLEWIGRIDPASGHTKYDPKFQVRATITTDT |
| | | STSTAYLELSSLRSEDTAVYYCARSGGLPDVWGQGTT |
| | | VTVSS |
| 1203 | AS12823 VH | QVQLVQSGAEVKKPGASVKLSCKASGFDICDTYMHW |
| | | VRQRPGQGLEWIGRIDPASGHTKYDPKFQVRATMTTD |
| | | TSTSTVYLELSSLRSEDTAVYYCARSGGLPDVWGQGT |
| | | TVTVSS |
| 1204 | AS12819 VH | QVQLVQSGAEVVKPGASVKLSCKASGFDICDTYMHW |
| | | VRQRPGQGLEWMGRIDPASGHTKYDPKFQVRVTMTT |
| | | DTSTSTVYLELSSLRSEDTAVYYCARSGGLPDVWGQG |
| | | TTVTVSS |
| 1205 | AS12816 VH | QVQLVQSGAEVKKPGASVKVSCKASGFDICDTYMHW |
| | | VKQRPGQGLEWIGRIDPASGHTKYDPKFQVRATITRDT |
| | | STSTAYLELSSLRSEDTAVYYCSRSGGLPDVWGQGTT |
| | | VTVSS |
| 1206 | AS12825 VH | QVQLVQSGAEVKKPGASVKVSCKASGFDICDTYMHW |
| | | VKQAPGQGLEWMGRIDPASGHTKYDPKFQVRATMTT |
| | | DTSTSTAYLELSSLRSEDTAVYYCSRSGGLPDVWGQG |
| | | TTVTVSS |
| 1207 | 12835 VH | QVQLVQSGAEVKKPGASVKLSCKASGFDIQDTYMHW |
| | | VRQAPGQGLEWMGRIDPASGHTKYDPKFQVRVTMTT |
| | | DTSTSTVYMELSSLRSEDTAVYYCSRSGGLPDVWGQG |
| | | TTVTVSS |
| 1208 | 18-7 VH | QVQLVQSGAEVKKPGASVKLSCKASGFDIQDTYMHW |
| | | VRQAPGQGLEWMGRIDPASGHTKYDPKFQVRVTMTR |
| | | DTSTSTVYMELSSLRSEDTAVYYCSRSGGLPDVWGQG |
| | | TTVTVSS |
| 1209 | 21-3 VH, L8 VH | QVQLVQSGAEVKKPGASVKVSCKASGFDIQDTYMHW |
| | | VRQAPGQGLEWMGRIDPASGHTKYDPKFQVRVTMTR |
| | | DTSTSTVYMELSSLRSEDTAVYYCARSGGLPDVWGQG |
| | | TTVTVSS |
| 1210 | 21-3 V102K VH | QVQLVQSGAEVKKPGASVKVSCKASGFDIQDTYMHW |
| | | VRQAPGQGLEWMGRIDPASGHTKYDPKFQVRVTMTR |
| | | DTSTSTVYMELSSLRSEDTAVYYCARSGGLPDKWGQG |
| | | TTVTVSS |
| 1211 | 21-3 V102M VH | QVQLVQSGAEVKKPGASVKVSCKASGFDIQDTYMHW |
| | | VRQAPGQGLEWMGRIDPASGHTKYDPKFQVRVTMTR |
| | | DTSTSTVYMELSSLRSEDTAVYYCARSGGLPDMWGQ |
| | | GTTVTVSS |
| 1212 | 21-3 V102Q VH | QVQLVQSGAEVKKPGASVKVSCKASGFDIQDTYMHW |
| | | VRQAPGQGLEWMGRIDPASGHTKYDPKFQVRVTMTR |
| | | DTSTSTVYMELSSLRSEDTAVYYCARSGGLPDQWGQG |
| | | TTVTVSS |
| 1213 | 21-3 V102W VH | QVQLVQSGAEVKKPGASVKVSCKASGFDIQDTYMHW |
| | | VRQAPGQGLEWMGRIDPASGHTKYDPKFQVRVTMTR |
| | | DTSTSTVYMELSSLRSEDTAVYYCARSGGLPDWWGQ |
| | | GTTVTVSS |
| 1214 | 21-3 CDRv VH | QVQLVQSGAEVKKPGASVKVSCKASGFX₁X₂X₃DTX₄X₅ |
| | | HWVRQAPGQGLEWMGRIDPASGHTKYDPKFQVRVT |
| | | MTRDTSTSTVYMELSSLRSEDTAVYYCARSGGX₆PDX₇ |
| | | WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 1215 | 21-3 CDRv VH | QVQLVQSGAEVKKPGASVKVSCKASGFX₁X₂X₃DTX₄X₅ |
| | | HWVRQAPGQGLEWMGRIDPASGHTKYDPKFQVRVT |
| | | MTRDTSTSTVYMELSSLRSEDTAVYYCSRSGGX₆PDX₇ |
| | | WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 1216 | Clone 2 VH, clone | QVQLVQSGAEVKKPGASVKVSCKASGFDIQDTYMHW |
| | 52 VH | VRQAPGQGLEWMGRIEPASGHIKYSPKFQGRVTMTRD |
| | | TSTSTVYMELSSLRSEDTAVYYCARSGGLPDWWGQG |
| | | TTVTVSS |
| 1217 | Clone 46 VH | QVQLVQSGAEVKKPGASVKVSCKASGFDIQDTYMHW |
| | | VRQAPGQGLEWMGRIEPASGHVKYSPKFQVRVTMTR |
| | | DTSTSTVYMELSSLRSEDTAVYYCARSGGLPDWWGQ |
| | | GTTVTVSS |
| 1218 | Clone 47 VH | QVQLVQSGAEVKKPGASVKVSCKASGFDIQDTYMHW |
| | | VRQAPGQGLEWMGRIEPASGHVKYDPKFQTRVTMTR |
| | | DTSTSTVYMELSSLRSEDTAVYYCARSGGLPDWWGQ |
| | | GTTVTVSS |
| 1219 | Clone 14 VH | QVQLVQSGAEVKKPGASVKVSCKASGFDIQDTYMHW |
| | | VRQAPGQGLEWMGRIDPASGHIKYDPKFQKRVTMTRD |
| | | TSTSTVYMELSSLRSEDTAVYYCARSGGLPDMWGQGT |
| | | TVTVSS |
| 1220 | Clone 16L VH | QVQLVQSGAEVKKPGASVKVSCKASGFDIQDTYMHW |
| | | VRQAPGQGLEWMGRIDPASGHvKiDPKFQVRVTMTRD |
| | | TSTSTVYMELSSLRSEDTAVYYCARSGGLPDMWGQGT |
| | | TVTVSS |
| 1221 | Clone 17L VH | QVQLVQSGAEVKKPGASVKVSCKASGFDIQDTYMHW |
| | | VRQAPGQGLEWMGRIDPASGHLKYDPKFQVRVTMTR |
| | | DTSTSTVYMELSSLRSEDTAVYYCARSGGLPDMWGQ |
| | | GTTVTVSS |
| 1222 | Clone 17L-1 VH | QVQLVQSGAEVKKPGASVKVSCKASGFDIQDTYMHW |
| | | VRQAPGQGLEWMGRIDPASGHLKYDPKFQRRVTMTR |
| | | DTSTSTVYMELSSLRSEDTAVYYCARSGGLPDMWGQ |
| | | GTTVTVSS |
| 1223 | Clone 23 VH, clone | QVQLVQSGAEVKKPGASVKVSCKASGFDIQDTYMHW |
| | A1 VH | VRQAPGQGLEWMGRIDPASGHLKYDPKFQNRVTMTR |
| | | DTSTSTVYMELSSLRSEDTAVYYCARSGGLPDKWGQG |
| | | TTVTVSS |
| 1224 | Clone 53 VH, clone | QVQLVQSGAEVKKPGASVKVSCKASGFDIQDTYMHW |
| | E1 VH | VRQAPGQGLEWMGRIEPASGHLKYDPKFQERVTMTR |
| | | DTSTSTVYMELSSLRSEDTAVYYCARSGGLPDKWGQG |
| | | TTVTVSS |
| 1225 | Clone 3-17L V-A | QVQLVQSGAEVKKPGASVKVSCKASGFDIQDTYMHW |
| | VH | VRQAPGQGLEWMGRIDPASGHLKYDPKFQGRVTITRD |
| | | TSASTAYMELSSLRSEDTAVYYCARSGGLPDMWGQG |
| | | TTVTVSS |
| 1226 | Clone 3-17L VH | QVQLVQSGAEVKKPGASVKVSCKASGFDIQDTYMHW |
| | | VRQAPGQGLEWMGRIDPASGHLKYDPKFQGRVTITRD |
| | | TSASTVYMELSSLRSEDTAVYYCARSGGLPDMWGQG |
| | | TTVTVSS |
| 1227 | Clone L8mod VH | QVQLVQSGAEVKKPGASVKVSCKASGFDIQDTYMHW |
| | | VRQAPGQGLEWMGRIDPASGHTKYDPKFQGRATITTD |
| | | TSASTAYLQLSSLRSEDTAVYYCARSGGLPDVWGQGT |
| | | TVTVSS |
| 1228 | Clone X-V VH | QVQLVQSGAEVKKPGASVKVSCKASGFDIQDTYMHW |
| | | VRQAPGQGLEWMGRIDPASGHTKYDPKFQVRATITTD |
| | | TSASTAYLQLSSLRSEDTAVYYCARSGGLPDFWGQGT |
| | | TVTVSS |
| 1229 | Clone X VH, clone | QVQLVQSGAEVKKPGASVKVSCKASGFDIQDTYMHW |
| | XL3-6 VH, clone | VRQAPGQGLEWMGRIDPASGHTKYDPKFQGRATITTD |
| | XL3-10 VH, clone | TSASTAYLQLSSLRSEDTAVYYCARSGGLPDFWGQGT |
| | XL3-15 VH, clone | TVTVSS |
| | L3-13 VH | |
| 1230 | Clone H3-1 VH | QVQLVQSGAEVKKPGASVKVSCKASGFDIQDTYMHW |
| | | VRQAPGQGLEWMGRIDPASGHTKYDPKFQGRATITTD |
| | | TSASTAYLQLSSLRSEDTAVYYCARSGGLPDLWGQGT |
| | | TVTVSS |
| 1231 | Clone H2-2 VH | QVQLVQSGAEVKKPGASVKVSCKASGFDIQDTYMHW |
| | | VRQAPGQGLEWMGRIDPASGHSKYDPKFQVRATITTD |
| | | TSASTAYLQLSSLRSEDTAVYYCARSGGLPDFWGQGT |
| | | TVTVSS |
| 1232 | Clone H2-5 VH | QVQLVQSGAEVKKPGASVKVSCKASGFDIQDTYMHW |
| | | VRQAPGQGLEWMGRIDPASGHYKYDPKFQVRATITTD |
| | | TSASTAYLQLSSLRSEDTAVYYCARSGGLPDFWGQGT |
| | | TVTVSS |
| 1233 | M1 VH | EVQLVESGGGLVQPGGSLRLSCAVSGFTFSSYWMSWV |
| | | RQAPGKGLEWVANIKEDGSEKNYVDSVKGRFTLSSDN |
| | | AKNSLYLQMNSLRAEDTAVYYCAREDYDSYYKYGM |
| | | DVWGQGTAVIVSS |
| 1234 | M2 VH | QVQLQQWGAGLLKPSETLSLTCAVYGGSFTGFYWSWI |
| | | RQPPGKGLEWIGEINHRGNTNYNPSLKSRVTMSVDTS |
| | | KNQFSLNMISVTAADTAMYFCASPFYDFWSGSDYWG |
| | | QGTLVTVSS |
| 1235 | M3 VH | QVQLVQSGAEVKKPGASVKVSCKASGYDFTYYGISW |
| | | VRQAPGQGLEWMGWISTYNGNTHYARMLQGRVTMT |
| | | TDTSTRTAYMELRSLRSDDTAVYYCARENYYGSGAY |
| | | RGGMDVWGQGTTVTVSS |
| 1236 | M3 VH + constant | QVQLVQSGAEVKKPGASVKVSCKASGYDFTYYGISW |
| | | VRQAPGQGLEWMGWISTYNGNTHYARMLQGRVTMT |
| | | TDTSTRTAYMELRSLRSDDTAVYYCARENYYGSGAY |
| | | RGGMDVWGQGTTVTVSSASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPEAAGAPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALHN |
| | | HYTQKSLSLSPG |
| 1237 | M4 VH | QLQLQESGPGLVKPSETLSLTCTVSGGSISSRSYYWGW |
| | | IRQPPGKGLEWIGSIYYNGRTYYNPSLKSRVTISVDTSK |
| | | NQFSLKLSSVTAADTAVYYCAREDYGDYGAFDIWGQ |
| | | GTMVTVSS |
| 1238 | M5 VH | QVTLKESGPALVKPTQTLTLTCTFSGFSLSTSNMGVVW |
| | | IRQPPGKALEWLAHILWDDREYSNPALKSRLTISKDTS |
| | | KNQVVLTMTNMDPVDTATYYCARMSRNYYGSSYVM |
| | | DYWGQGTLVTVSS |
| 1239 | M6 VH | QVQLVQSGSELKKPGASVKVSCKASGYTFTLYGMNW |
| | | VRQAPGQGLEWMGWINTYTGEPTYADDFKGRFVFSL |
| | | DTSVSTAYLQISSLKAEDTAVYYCAR |
| | | DTAMDYAMAYWGQGTLVTVSS |
| 1240 | M6 VH | QVQLVQSGSELKKPGASVKVSCKASGYTFTLYGMNW |
| | | VKQAPGKGLKWMGWINTYTGEPTYADDFKGRFVFSL |
| | | DTSVSTAYLQISSLKAEDTAVYFCAR |
| | | DTAMDYAMAYWGQGTLVTVSS |
| 1241 | M6 VH | QVQLVQSGSELKKPGASVKVSCKASGYTFTNYGMNW |
| | | VRQAPGQGLEWMGWINTYTGEPTYADDFKGRFVFSL |
| | | DTSVSTAYLQISSLKAEDTAVYYCAR |
| | | DYGKYGDYYAMDYWGQGTLVTVSS |
| 1242 | M6 VH | QVQLVQSGSELKKPGASVKVSCKASGYTFTNYGMNW |
| | | VRQAPGKGLKWMGWINTYTGEPTYADDFKGRFVFSL |
| | | DTSVSTAYLQISSLKAEDTAVYFCAR |
| | | DYGKYGDYYAMDYWGQGTLVTVSS |
| 1243 | M7 VH | QVQLQQPGSVLVRPGASVKVSCKASGYTFTSSWMHW |
| | | AKQRPGQGLEWIGEIHPNSGGTNYNEKFKGKATVDTS |
| | | SSTAYVDLSSLTSEDSAVYYCARGDYYGYVSWFAYW |
| | | GQGTLVTVSS |
| 1244 | M7 VH | QVQLVQSGAEVKKPGASVKVSCKASGYTFTSSWMHW |
| | | ARQAPGQGLEWIGEIHPNSGGTNYAQKFQGRATLTVD |
| | | TSSSTAYMELSRLRSDDTAVYYCARGDYYGYVSWFA |
| | | YWGQGTLVTVSS |
| 1245 | M7 VH | QVQLVQSGAEVKKPGASVKVSCKASGYTFTSSWMHW |
| | | ARQAPGQGLEWIGEIHPNSGGTNYAQKFQGRATMTV |
| | | DTSISTAYMELSRLRSDDTAVYYCARGDYYGYVSWFA |
| | | YWGQGTLVTVSS |
| 1246 | M7 VH | QVQLVQSGAEVKKPGASVKVSCKASGYTFTSSWMHW |
| | | ARQAPGQGLEWIGEIHPNSGGTNYAQKFQGRVTMTV |
| | | DTSISTAYMELSRLRSDDTAVYYCARGDYYGYVSWFA |
| | | YWGQGTLVTVSS |
| 1247 | M7 VH | QVQLVQSGAEVKKPGASVKVSCKASGYTFTSSWMHW |
| | | ARQAPGQGLEWMGEIHPNSGGTNYAQKFQGRVTMTV |
| | | DTSISTAYMELSRLRSDDTAVYYCARGDYYGYVSWFA |
| | | YWGQGTLVTVSS |
| 1248 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1249 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1250 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1251 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1252 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1253 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1254 | M8 VH | QVQLVQSGAEVKKPGASVKVSCKASGYTFTSYDINW |
| | | VRQAPGQGLEWMGWLNPNSGNTGYAQKFQGRVTMT |
| | | ADRSTSTAYMELSSLRSEDTAVYYCAREVPETAAFEY |
| | | WGQGTLVTVSS |
| 1255 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1256 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1257 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1258 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1259 | M8 VH | QVQLVQSGAEVKKPGASVKVSCKASGYTFTSYDINW |
| | | VRQAPGQGLEWMGWLNPNSGYTGYAQKFQGRVTMT |
| | | ADRSTSTAYMELSSLRSEDTAVYYCAREVPETAAFEY |
| | | WGQGTLVTVSS |
| 1260 | M9 VH | QVQLQESGPGLVKPSETLSLTCTVSGGSISSYFWSWIR |
| | | QPPGKGLEWIGYIYYSGNTKYNPSLKSRVTISIDTSKNQ |
| | | FSLKLSSVTAADTAVYYCARETGSYYGFDYWGQGTL |
| | | VTVSS |
| 1261 | M10 VH | QVQLQQWGAGLLKPSETLSLTCAVHGGSFSGYYWNW |
| | | IRQPPGKGLEWIGEINHAGNTNYNPSLKSRVTISLDTSK |
| | | NQFSLTLTSVTAADTAVYYCARGYCRSTTCYFDYWG |
| | | QGTLVTVSS |
| 1262 | M11 VH | EVQLLESGGGLVQPGKSLRLSCAVSGFTFSTYGMNWV |
| | | RQAPGKGLEWVSSISGTGRTTYHADSVQGRFTVSRDN |
| | | SKNILYLQMNSLRADDTAVYFCTKERGDYYYGVFDY |
| | | WGQGTLVTVSS |
| 1263 | M12 VH | QIQLVQSGPELKKPGETVKISCKASGYTFTTYGMSWV |
| | | KQAPGKGLKWMGWMNTYSGVTTYADDFKGRFAFSL |
| | | ETSASTAYMQIDNLKNEDTATYFCAREGYVEDDYYAT |
| | | DYWGQGTSVTVSS |
| 301 | Variable Heavy 1 | X1VQLVQSGAEVKKPGASVKVSCKAS[HCDR1]WVX2 |
| | | QX3PGQGLEWX4G[HCDR2]RX5TX6TX7DTSTSTX8YX |
| | | 9ELSSLRSEDTAVYYCAR[HCDR3]WGQGTTVTVSS |
| | | wherein each of X1-X11 is independently |
| | | selected from A, R, N, D, C, Q, E, G, H, |
| | | I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 302 | Variable Heavy 2 | X1VQLVQSGAEVKKPGASVKVSCKAS[HCDR1]WVX2 |
| | | QX3PGQGLEWX4G[HCDR2]RX5TX6TX7DTSTSTX8YX |
| | | 9ELSSLRSEDTAVYYC[HCDR3]WGQGTTVTVSS |
| | | wherein each of X1-X11 is independently |
| | | selected from A, R, N, D, C, Q, E, G, H, |
| | | I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1301 | Variable Heavy 3 | X1VQLVQSGAEVKKPGASVKVSCKAS[HCDR1]WVX2 |
| | | QRPGQGLEWX4G[HCDR2]RX5TX6TX7DTSTSTX8YX9 |
| | | ELSSLRSEDTAVYYCAR[HCDR3]WGQGTTVTVSS |
| | | wherein each of X1, X2, X4, X5, X6, X7, |
| | | X8, X9, X10, and X11 is independently |
| | | selected from A, R, N, D, C, Q, E, G, |
| | | H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1302 | Variable Heavy 4 | X1VQLVQSGAEVKKPGASVKVSCKAS[HCDR1]WVX2 |
| | | QRPGQGLEWX4G[HCDR2]RX5TX6TX7DTSTSTX8YX9 |
| | | ELSSLRSEDTAVYYC[HCDR3]WGQGTTVTVSS |
| | | wherein each of X1, X2, X4, X5, X6, X7, |
| | | X8, X9, X10, and X11 is independently |
| | | selected from A, R, N, D, C, Q, E, G, |
| | | H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |

TABLE 17
Light Chain Variable Region (VL) Amino Acid Sequences

| SEQ | | |
|---|---|---|
| ID | | |
| NO | Description | Sequence |
| 201 | 217 VL, | EIVLTQSPGTLSLSPGERATLSCRASSSVSYMYW |
| | 219 VL, 221 VL, | YQQKPGQAPRPLIYATSNLASGIPDRFSGSGSGT |
| | 200 VL, 213 VL, | DFTLTISRLEPEDFAVYYCQQWEGNPRTFGGGT |
| | 212 VL, 211 VL, | KLEIK |
| | 199 VL, 214 VL, | |
| | 216 VL, 222 VL, | |
| | 203 VL, 147 VL, | |
| | 218 VL, 179 VL, | |
| | 148 VL, 149 VL, | |
| | 151 VL, 180 VL, | |
| | 175 VL, 178 VL, | |
| | 145 VL, 146 VL, | |
| | 150 VL, 152 VL, | |
| | 176 VL, 177 VL, | |
| | 201 VL, 202 VL, | |
| | 204 VL, 215 VL, | |
| | 224 VL | |
| 202 | 223 VL, 107 VL, | EIVLTQSPGTLSLSPGERATLSCRASSSVSYMYW |
| | 205 VL, 181 VL, | YQQKPGQAPRLLIYATSNLASGIPDRFSGSGSGT |
| | 188 VL, 64 VL, | DFTLTISRLEPEDFAVYYCQQWEGNPRTFGGGT |
| | 67 VL, 68 VL, | KLEIK |
| | 94 VL, 33 VL, 57 | |
| | VL, 58 VL, 59 VL, | |
| | 60 VL, 61 VL, 62 | |
| | VL, 63 VL, 65 VL, | |
| | 66 VL, 69 VL, 70 | |
| | VL, 71 VL, 72 VL, | |
| | 76 VL, 77 VL, 78 | |
| | VL, 91 VL, 92 VL, | |
| | 93 VL, 97 VL, 98 | |
| | VL, 99 VL, 140 | |
| | VL, 142 VL, 143 | |
| | VL, 182 VL, 183 | |
| | VL, 184 VL, 185 | |
| | VL, 186 VL, 187 | |
| | VL, 189 VL, 190 | |
| | VL, 191 VL, 192 | |
| | VL, 206 VL, 207 | |
| | VL, 208 VL, 209 | |
| | VL, 210 VL, 18-7 | |
| | S93E VL, clone 34 | |
| | VL, clone 2 VL, | |
| | clone 46 VL, clone | |
| | 47 VL, clone 23 | |
| | VL, clone 53 | |
| 203 | 15 VL, 18-7, L8, | EIVLTQSPGTLSLSPGERATLSCRASSSVSYMYW |
| | clone L8mod VL, | YQQKPGQAPRLLIYATSNLASGIPDRFSGSGSGT |
| | clone X-V VL, | DFTLTISRLEPEDFAVYYCQQWSGNPRTFGGGT |
| | clone X VL, clone | KLEIK |
| | H3-1 VL, clone | |
| | H2-2 VL, clone | |
| | H2-5 VL | |
| 204 | 30 VL, 100 VL, | EIVLTQSPGTLSLSPGERATLSCRASSSVSYMYW |
| | 129 VL, 122 VL, | YQQKPGQAPRLLIYATSNLASGIPDRFSGSGSGT |
| | 127 VL, 126 VL, | DFTLTISRLEPEDFAVYYCQQWKGNPRTFGGGT |
| | 160 VL, 157 VL, | KLEIK |
| | 159 VL, 158 VL, | |
| | 125 VL, 103 VL, | |
| | 101 VL, 102 VL, | |
| | 104 VL, 105 VL, | |
| | 121 VL, 123 VL, | |
| | 124 VL, 128 VL, | |
| | 144 VL, 161 VL, | |
| | 162 VL, 163 VL, | |
| | 164 VL, clone | |
| | L3-13 VL | |
| 205 | 110 VL, 197 VL, | EIVLTQSPGTLSLSPGERATLSCRASSSVSYMYWYQQK |
| | 112 VL, 169 VL, | PGQAPRPWIYATSNLASGIPDRFSGSGSGTDFTLTISRL |
| | 173 VL, 115 VL, | EPEDFAVYYCQQWEGNPRTFGGGTKLEIK |
| | 113 VL, 96 VL, | |
| | 196 VL, 172 VL, | |
| | 75 VL, 174 VL, | |
| | 109 VL, 198 VL, | |
| | 170 VL, 29 VL, 31 | |
| | VL, 32 VL, 73 VL, | |
| | 74 VL, 95 VL, 108 | |
| | VL, 111 VL, 114 | |
| | VL, 116 VL, 117 | |
| | VL, 118 VL, 119 | |
| | VL, 120 VL, 130 | |
| | VL, 153 VL, 154 | |
| | VL, 155 VL, 156 | |
| | VL, 165 VL, 166 | |
| | VL, 167 VL, 168 | |
| | VL, 171 VL, 193 | |
| | VL, 194 VL, 195 | |
| | VL, 220 VL | |
| 206 | 134 VL, 132 VL, | EIVLTQSPGTLSLSPGERATLSCRASSSVSYMYWYQQK |
| | 133 VL, 135 VL, | PGQAPRPLIYATSNLASGIPDRFSGSGSGTDFTLTISRLE |
| | 136 VL | PEDFAVYYCQQWKGNPRTFGGGTKLEIK |
| 207 | 138 VL, 137 VL, | EIVLTQSPGTLSLSPGERATLSCRASSSVSYMYWYQQK |
| | 139 VL, 141 VL, | PGQAPRLLIYATSNLASGIPDRFSGSGSGTDFTLTISRLE |
| | clone XL3-15 VL | PEDFAVYYCQQWSRNPRTFGGGTKLEIK |
| 208 | 34 VL, 35 VL, 36 | EIVLTQSPGTLSASPGERATMSCRASSSVSYMYWYQQ |
| | VL, 37 VL, 38 VL, | KPGQAPRPWIYATSNLASGVPDRFSGSGSGTDYTLTIS |
| | 39 VL, 40 VL, 41 | RVEPEDFAVYYCQQWSGNPRTFGGGTKLEIK |
| | VL, 42 VL, 43 VL, | |
| | 44 VL, 45 VL, 46 | |
| | VL, 47 VL, 53 VL, | |
| | 54 VL, 55 VL, 79 | |
| | VL, 81 VL, 82 VL, | |
| | 83 VL, AS12824 | |
| | VL | |
| 209 | 85 VL | EIVLTQSPGTLSLSPGERATLSCRASSSVSYMYWYQQK |
| | | PGQAPRLLIYATSNLASGVPDRFSGSGSGTDFTLTISRL |
| | | EPEDFAVYYCQQWEGNPRTFGGGTKLEIK |
| 210 | 48 VL | EIVLTQSPGTLSASPGERATLSCRASSSVSYMYWYQQK |
| | | PGQAPRPWIYATSNLASGVPDRFSGSGSGTDYTLTISR |
| | | VEPEDFAVYYCQQWSGNPRTFGGGTKLEIK |
| 211 | 49 VL | EIVLTQSPGTLSASPGERATMSCRASSSVSYMYWYQQ |
| | | KPGQAPRLLIYATSNLASGVPDRFSGSGSGTDYTLTISR |
| | | VEPEDFAVYYCQQWSGNPRTFGGGTKLEIK |
| 212 | 50 VL | EIVLTQSPGTLSASPGERATMSCRASSSVSYMYWYQQ |
| | | KPGQAPRPWIYATSNLASGVPDRFSGSGSGTDFTLTISR |
| | | VEPEDFAVYYCQQWSGNPRTFGGGTKLEIK |
| 213 | 51 VL | EIVLTQSPGTLSASPGERATMSCRASSSVSYMYWYQQ |
| | | KPGQAPRPWIYATSNLASGVPDRFSGSGSGTDYTLTIS |
| | | RLEPEDFAVYYCQQWSGNPRTFGGGTKLEIK |
| 214 | 52 VL | EIVLTQSPGTLSASPGERATMSCRASSSVSYMYWYQQ |
| | | KPGQAPRPWIYATSNLASGVPDRFSGSGSGTDFTLTISR |
| | | LEPEDFAVYYCQQWSGNPRTFGGGTKLEIK |
| 215 | 56 VL | EIVLTQSPGTLSASPGERATMSCRASSSVSYMYWYQQ |
| | | KPGQAPRPWIYATSNLASGIPDRFSGSGSGTDYTLTISR |
| | | VEPEDFAVYYCQQWSGNPRTFGGGTKLEIK |
| 216 | 86 VL | EIVLTQSPGTLSLSPGERATLSCRASSSVSYMYWYQQK |
| | | PGQAPRLLIYATSNLASGIPDRFSGSGSGTDYTLTISRLE |
| | | PEDFAVYYCQQWEGNPRTFGGGTKLEIK |
| 217 | 87 VL | EIVLTQSPGTLSLSPGERATLSCRASSSVSYMYWYQQK |
| | | PGQAPRLLIYATSNLASGIPDRFSGSGSGTDFTLTISRVE |
| | | PEDFAVYYCQQWEGNPRTFGGGTKLEIK |
| 218 | 88 VL | EIVLTQSPGTLSLSPGERATLSCRASSSVSYMYWYQQK |
| | | PGQAPRLLIYATSNLASGIPDRFSGSGSGTDYTLTISRV |
| | | EPEDFAVYYCQQWEGNPRTFGGGTKLEIK |
| 219 | 89 VL | EIVLTQSPGTLSASPGERATLSCRASSSVSYMYWYQQK |
| | | PGQAPRLLIYATSNLASGIPDRFSGSGSGTDFTLTISRLE |
| | | PEDFAVYYCQQWEGNPRTFGGGTKLEIK |
| 220 | 90 VL | EIVLTQSPGTMSLSPGERATLSCRASSSVSYMYWYQQ |
| | | KPGQAPRLLIYATSNLASGIPDRFSGSGSGTDFTLTISRL |
| | | EPEDFAVYYCQQWEGNPRTFGGGTKLEIK |
| 1264 | 5C3D11 VL | QIVLSQSPAILSASPGEKVTMTCRASSSVSYMYWYQQ |
| | | KPGSSPKPWIYATSNLASGVPDRFSGSGSGTSYSLTISR |
| | | VEAEDAATYYCQQWSGNPRTFGGGTKLEIK |
| 1265 | 9E12E5 VL | MKLPVRLLVLMFWIPASSSDVLMTQTPLSLPVSLGDQ |
| | | ASISCRSSQTIVHSNGDTYLDWFLQKPGQSPKLLIYKVS |
| | | NRFSGVPDRFSGSGSGTDFTLKISRVEAEDLGVYYCFQ |
| | | GSHVPYTFGGGTKLEIK |
| 1266 | AS12823 VL | EIVLTQSPGTLSLSPGERATMSCRASSSVSYMYWYQQ |
| | | KPGQAPRPWIYATSNLASGIPDRFSGSGSGTDFTLTISR |
| | | VEPEDFAVYYCQQWSGNPRTFGGGTKLEIK |
| 1267 | AS12819 VL | EIVLTQSPGTLSLSPGERVTMSCRASSSVSYMYWYQQ |
| | | KPGQAPRPWIYATSNLASGVPDRFSGSGSGTDFTLTISR |
| | | VEPEDFAVYYCQQWSGNPRTFGGGTKVEIK |
| 1268 | AS12816 VL | EIVLTQSPGTLSASPGERVTLSCRASSSVSYMYWYQQK |
| | | PGQAPRPWIYATSNLASGVPDRFSGSGSGTDFTLTISRL |
| | | EPEDFAVYYCQQWSGNPRTFGGGTKLEIK |
| 1269 | AS12825 VL | EIVLTQSPGTLSASPGERVTMSCRASSSVSYMYWYQQ |
| | | KPGQAPRLLIYATSNLASGVPDRFSGSGSGTDFTLTISR |
| | | VEPEDFAVYYCQQWSGNPRTFGGGTKLEIK |
| 1270 | 12835 VL | EIVLTQSPGTLSLSPGERVTMSCRASSSVSYMYWYQQ |
| | | KPGQAPRPWIYATSNLASGVPDRFSGSGSGTDYTLTIS |
| | | RLEPEDFAVYYCQQWSGNPRTFGGGTKLEIK |
| 1271 | 21-3 VL | EIVLTQSPGTLSLSPGERATLSCRASSSVSYMYWYQQK |
| | | PGQAPRLLIYATSNLASGVPDRFSGSGSGTDYTLTISRL |
| | | EPEDFAVYYCQQWSGNPRTFGGGTKLEIK |
| 1272 | 18-7 S92D VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWDGNPRTFGGGTKLEIK |
| 1273 | 18-7 S92H VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWHGNPRTFGGGTKLEIK |
| 1274 | 18-7 S92N VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWNGNPRTFGGGTKLEIK |
| 1275 | 18-7 S92Q VL, | EIVLTQSPGTLSLSPGERATLSCRASSSVSYMYWYQQK |
| | clone 14 VL, clone | PGQAPRLLIYATSNLASGIPDRFSGSGSGTDFTLTISRLE |
| | 16L VL, clone 17L | PEDFAVYYCQQWQGNPRTFGGGTKLEIK |
| | VL, clone 17L-1 | |
| | VL, clone 3-17L V- | |
| | A VL, clone 3-17L | |
| | VL | |
| 1276 | 18-7 CDRv VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCX₁QWX₂X₃X₄PRTFGGGTKLEIK |
| | | X₁ = Q or N |
| | | X₂ = D, E, H, N, Q, or S |
| | | X₃ = A or G |
| | | X₄ = D, F, K, N, R, S, or T |
| 1277 | Clone 52 VL, clone | EIVLTQSPGTLSLSPGERATLSCGASSSVSYMYWYQQK |
| | A1 VL, clone E1 | PGQAPRLLIYATSNLASGIPDRFSGSGSGTDFTLTISRLE |
| | VL | PEDFAVYYCQQWEGNPRTFGGGTKLEIK |
| 1278 | Clone XL3-6 VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCSQWSGNPRTFGGGTKLEIK |
| 1279 | Clone XL3-10 VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWSGNPRSFGGGTKLEIK |
| 1280 | M1 VL | DIVMTQSPDSLAVSLGERATINCKSSQSILYSSNNKNYL |
| | | AWYQQKPGQPPKLLIYWASTRESGVPDRFSGSGSGTD |
| | | FTLTISSLQAEDVSVYYCQQYYSTPFTFGPGTKVDIK |
| 1281 | M2 VL | DIMLTQTPLTSPVTLGQPASISCKSSQSLVHSDGNTYLS |
| | | WLQQRPGQPPRLLFYKISNRFSGVPDRFSGSGAGTDFT |
| | | LKISRVEAEDVGVYYCMQATQFPLTFGGGTKVEIK |
| 1282 | M3 VL | EIVLTQSPATLSLSPGERATLSCRASQSVSSYLAWYQQ |
| | | KPGQAPRLLIYDASNRATGIPARFSGSGSGTDFTLTISSL |
| | | EPEDFAVYYCQQRSNWPWTFGQGTKVEIK |
| 1283 | M3 VL | EIVLTQSPATLSLSPGERATLSCRASQSVSSYLAWYQQ |
| | | KPGQAPRLLIYDASNRATGIPARFSGSGSGTDFTLTISSL |
| | | EPEDFAVYYCQQRSNWPWTFGQGTKVEIKRTVAAPSV |
| | | FIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 1284 | M4 VL | AIQLTQSPSSLSASVGDRVTITCRASQGISSALAWYQQ |
| | | KPGKAPKLLIYDASSLESGVPSRFSGSGSGTDFTLTISSL |
| | | QPEDFATYYCQQFNSYPLTFGGGTKVEIK |
| 1285 | M5 VL | DIQLTQSPSFLSASVGDRVTITCSASSSVNYMHWYQQK |
| | | PGKAPKLLIYSTSNLASGVPSRFSGSGSGTEFTLTISSLQ |
| | | PEDFATYYCHQWNNYGTFGQGTKVEIKR |
| 1286 | M6 VL | DVVMTQSPLSLPVTLGQPASISCKSSQNIVHSDGNTYL |
| | | EWFQQRPGQSPRRLIYKVSNRFSGVPDRFSGSGSGTDF |
| | | TLKISRVEAEDVGVYYCFQGSHVPLTFGGGTKVEIKR |
| 1287 | M6 VL | DVVMTQSPLSLPVTLGQPASISCKSSQNIVHSDGNTYL |
| | | EWFQQRPGQSPRRLIYKVSNRFSGVPDRFSGSGSGTDF |
| | | TLKISRVEAEDVGVYYCFQGSHVPLTFGQGTKVEIKR |
| 1288 | M6 VL | DVVMTQTPLSLPVTPGEPASISCKSSQNIVHSDGNTYLE |
| | | WYLQKPGQSPQLLIYKVSNRFSGVPDRFSGSGSGTDFT |
| | | LKISRVEAEDLGVYYCFQGSHVPLTFGGGTKVEIKR |
| 1289 | M6 VL | DVVMTQTPLSLPVSLGDQASISCKSSQNIVHSDGNTYL |
| | | EWYLQKPGQSPKVLIYKVSNRFSGVPDRFSGSGSGTDF |
| | | TLKISRVEAEDLGVYYCFQGSHVPLTFGGGTKVEIKR |
| 1290 | M6 VL | DVVMTQSPLSLPVTLGQPASISCRSSQSIVHSNGNTYLD |
| | | WFQQRPGQSPRRLIYKVSNRFSGVPDRFSGSGSGTDFT |
| | | LKISRVEAEDVGVYYCFQGSHVPLTFGGGTKVEIKR |
| 1291 | M6 VL | DVVMTQSPLSLPVTLGQPASISCRSSQSIVHSNGNTYLD |
| | | WFQQRPGQSPRRLIYKVSNRFSGVPDRFSGSGSGTDFT |
| | | LKISRVEAEDVGVYYCFQGSHVPLTFGQGTKVEIKR |
| 1292 | M6 VL | DVVMTQTPLSLPVTPGEPASISCRSSQSIVHSNGNTYLD |
| | | WYLQKPGQSPQLLIYKVSNRFSGVPDRFSGSGSGTDFT |
| | | LKISRVEAEDLGVYYCFQGSHVPLTFGGGTKVEIKR |
| 1293 | M6 VL | DVVMTQTPLSLPVSLGDQASISCRSSQSIVHSNGNTYL |
| | | DWYLQKPGQSPKVLIYKVSNRFSGVPDRFSGSGSGTDF |
| | | TLKINRVEAEDLGVYFCFQGSHVPLTFGGGTKLEIKR |
| 1294 | M7 VL | DIQMNQSPSSLSASLGDTITITCHASQNINVLLSWYQQK |
| | | PGNIPKLLIYKASNLHTGVPSRFSGSGSGTGFTFTISSLQ |
| | | PEDIATYYCQQGQSYPYTFGGGTKLEIK |
| 1295 | M7 VL | DIQMTQSPSSLSASVGDRVTITCQASQDISNYLNWYQQ |
| | | KPGKAPKLLIYDASNLETGVPSRFSGSGSGTDFTFTISS |
| | | LQPEDIATYYCQQYDNLPYTFGQGTKLEIK |
| 1296 | M7 VL | DIQMTQSPSSLSASVGDRVTITCQASQNINVLLNWYQQ |
| | | KPGKAPKLLIYKASNLHTGVPSRFSGSGSGTDFTFTISS |
| | | LQPEDIATYYCQQGQSYPYTFGQGTKLEIK |
| 1297 | M7 VL | DIQMNQSPSSLSASVGDRVTITCQASQNINVLLSWYQQ |
| | | KPGKAPKLLIYKASNLHTGVPSRFSGSGSGTDFTFTISS |
| | | LQPEDIATYYCQQGQSYPYTFGQGTKLEIK |
| 1298 | M8 VL | QSVLTQPPSVSGAPGQRVTISCTSSSSDIGAXXGVXWY |
| | | QQLPGTAPKLLIEGYYNRPSGVPDRFSGSKSGTSASLTI |
| | | TGLLPEDEGDYYCQSXDGTLSALFGGGTKLTVLG |
| 1299 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1300 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSYDGTLSALFGGGTKLTVLG |
| 1304 | M8 VL | QSVLTQPPSVSGAPGQRVTISCTSSSSDIGAALGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1305 | M8 VL | QSVLTQPPSVSGAPGQRVTISCTSSSSDIGAGSGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1306 | M8 VL | QSVLTQPPSVSGAPGQRVTISCTSSSSDIGAGQGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1307 | M8 VL | QSVLTQPPSVSGAPGQRVTISCTSSSSDIGAGLGVLWY |
| | | QQLPGTAPKLLIEGYYNRPSGVPDRFSGSKSGTSASLTI |
| | | TGLLPEDEGDYYCQSWDGTLSALFGGGTKLTVLG |
| 1308 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1309 | M8 VL | QSVLTQPPSVSGAPGQRVTISCTSSSSDIGAGSGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1310 | M8 VL | QSVLTQPPSVSGAPGQRVTISCTSSSSDIGAGQGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 1311 | M8 VL | QSVLTQPPSVSGAPGQRVTISCTSSSSDIGAGLGVLWY |
| | | QQLPGTAPKLLIEGYYNRPSGVPDRFSGSKSGTSASLTI |
| | | TGLLPEDEGDYYCQSWDGTLSALFGGGTKLTVLG |
| 1312 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSFDGTLSALFGGGTKLTVLG |
| 1313 | M9 VL | DIQMTQSPSSLSASVGDRVTITCRASQSINNYLNWYQQ |
| | | RPGKAPKLLIYAASSLQSGVPSRFSGSGSGTDFTLTISSL |
| | | QPGDFATYYCQQSYSTPRTFGQGTKLEIK |
| 1314 | M10 VL | EIVLTQSPGTLSLSPGERATLSCRASQSVRSSYLAWYQ |
| | | QKPGQAPRLLIYGASSRATGIPDRFSGSGSGTDFTLTIS |
| | | RLEPEDFAVYYCQQYGSSPTFGQGTRLEIK |
| 1315 | M11 VL | DIQMTQSPSTLSASVGDRVTITCRASQTISSWLAWYQQ |
| | | TPEKAPKLLIYAASNLQSGVPSRFSGSGSGTEFTLTISSL |
| | | QPDDFATYYCQQYHRSWTFGQGTKVEIT |
| 1316 | M12 VL | DVLMTQTPLSLPVSLGDQASISCRSSQNIVHSDGNTYL |
| | | EWYLQKPGQSPKLLIYKVSNRFSGVPDRFSGSGSGTDF |
| | | TLKISRVEAEDLGIYYCFQGSHVPLTFGAGTKLELK |
| 303 | Variable Light | EIVLTQSPGTLSLSPGERATLSC[LCDR1]WYQQKPGQA |
| | | PRX10X11IY[LCDR2]GIPDRFSGSGSGTDFTLTISRLEPE |
| | | DFAVYYC[LCDR3]FGGGTKLEIK |
| | | wherein each of X10 and X11 is independently |
| | | selected from A, R, N, D, C, Q, E, G, H, I, |
| | | L, K, M, F, P, S, T, W, Y, or V |
| | | In some cases, X10 = L or P |
| | | In some cases, X11 = L or W |

TABLE 18A
Additional Sequences

| SEQ | | |
|---|---|---|
| ID | | |
| NO | Description | Sequence |
| 304 | 219 HC FR1, | QVQLVQSGAEVKKPGASVKVSCKAS |
| | 212 HC FR1 | |
| 305 | 219 HC FR2 | WVKQRPGQGLEWMG |
| 313 | 212 HC FR2 | WVRQRPGQGLEWIG |
| 1317 | HC FR2 | WVRQAPGQGLEWMG |
| 306 | 219 HC FR3a | RVTITRDTSTSTVYLELSSLRSEDTAVYYCAR |
| 307 | 219 HC FR3b | RVTITRDTSTSTVYLELSSLRSEDTAVYYC |
| 314 | 212 HC FR3a | RATITTDTSTSTAYLELSSLRSEDTAVYYCAR |
| 315 | 212 HC FR3b | RATITTDTSTSTAYLELSSLRSEDTAVYYC |
| 1318 | HC FR3 | RVTMTRDTSTSTVYMELSSLRSEDTAVYYC |
| 1319 | HC FR3 | RATITTDTSASTAYLQLSSLRSEDTAVYYC |
| 1320 | HC FR3 | RVTITRDTSASTVYMELSSLRSEDTAVYYC |
| 1321 | HC FR3 | RVTITRDTSASTAYMELSSLRSEDTAVYYC |
| 1322 | HC FR3 | RVTMTRDTSTSTVYMELSSLRSEDTAVYYCSR |
| 1323 | HC FR3 | RVTMTRDTSTSTVYMELSSLRSEDTAVYYCAR |
| 308 | 219 HC FR4, | WGQGTTVTVSS |
| | 212 HC FR4 | |
| 309 | 219 LC FR1, | EIVLTQSPGTLSLSPGERATLSC |
| | 212 LC FR1 | |
| 310 | 219 LC FR2, | WYQQKPGQAPRPLIY |
| | 212 LC FR2 | |
| 1324 | LC FR2 | WYQQKPGQAPRLLIY |
| 311 | 219 LC FR3, | GIPDRFSGSGSGTDFTLTISRLEPEDFAVYYC |
| | 212 LC FR3 | |
| 1325 | LC FR3 | GVPDRFSGSGSGTDYTLTISRLEPEDFAVYYC |
| 312 | 219 LC FR4, | FGGGTKLEIK |
| | 212 LC FR4 | |
| 316 | IGHV1-46*02 | QVQLVQSGAEVKKPGASVKVSCKASGYTFNSYYMHW |
| | | VRQAPGQGLEWMGIINPSGGSTSYAQKFQGRVTMTRD |
| | | TSTSTVYMELSSLRSEDTAVYYCAR |
| 317 | IGKV3-20*01 | EIVLTQSPGTLSLSPGERATLSCRASQSVSSSYLAWYQ |
| | | QKPGQAPRLLIYGASSRATGIPDRFSGSGSGTDFTLTIS |
| | | RLEPEDFAVYYCQQYGSSP |
| 319 | Light Chain | RTVAAPSVFIFPPSDEQLKSGTASVVCLLNNFYPREAK |
| | Constant | VQWKVDNALQSGNSQESVTEQDSKDSTYSLSSTLTLS |
| | | KADYEKHKVYACEVTHQGLSSPVTKSFNRGEC |

TABLE 18B
Additional sequences

| SEQ | |
|---|---|
| ID | |
| NO | Sequence |
| 3001 | GFDIQDTYMH |
| 3002 | RIDPASGHTKYDPKFQV |
| 3003 | RIEPASGHIKYDPKFQG |
| 3005 | RIEPASGHIKYDPKFQV |
| 3006 | SGGLPDV |
| 3008 | SGGLPDW |
| 3010 | RASSSVSYMY |
| 3011 | ATSNLAS |
| 3012 | QQWEGNPRT |
| 3013 | QQWKGNPRT |
| 3116 | EVQLVQSGAEVKKPGASVKVSCKASGFDIQDTYMHWVKQAPGQGLEW |
| | MGRIEPASGHIKYDPKFQGRVTMTRDTSTSTVYMELSSLRSEDTAVYYC |
| | ARSGGLPDWWGQGTTVTVSS |
| 3121 | QVQLVQSGAEVKKPGASVKVSCKASGFDIQDTYMHWVRQAPGQGLEW |
| | MGRIEPASGHIKYDPKFQVRVTMTRDTSTSTVYMELSSLRSEDTAVYYC |
| | ARSGGLPDWWGQGTTVTVSS |
| 3122 | QVQLVQSGAEVKKPGASVKVSCKASGFDIQDTYMHWVRQAPGQGLEW |
| | MGRIEPASGHIKYDPKFQVRATMTRDTSTSTVYMELSSLRSEDTAVYYC |
| | ARSGGLPDWWGQGTTVTVSS |
| 3123 | QVQLVQSGAEVKKPGASVKVSCKASGFDIQDTYMHWVRQAPGQGLEW |
| | MGRIEPASGHIKYDPKFQVRVTITRDTSTSTVYMELSSLRSEDTAVYYCA |
| | RSGGLPDWWGQGTTVTVSS |
| 3124 | QVQLVQSGAEVKKPGASVKVSCKASGFDIQDTYMHWVRQAPGQGLEW |
| | MGRIEPASGHIKYDPKFQVRATITRDTSTSTVYMELSSLRSEDTAVYYCA |
| | RSGGLPDWWGQGTTVTVSS |
| 3125 | QVQLVQSGAEVKKPGASVKVSCKASGFDIQDTYMHWVKQAPGQGLEW |
| | MGRIEPASGHIKYDPKFQGRVTMTRDTSTSTVYMELSSLRSEDTAVYYC |
| | ARSGGLPDWWGQGTTVTVSS |
| 3128 | QVQLVQSGAEVKKPGASVKVSCKASGFDIQDTYMHWVRQAPGQGLEW |
| | MGRIEPASGHIKYDPKFQGRATMTRDTSTSTVYMELSSLRSEDTAVYYC |
| | ARSGGLPDWWGQGTTVTVSS |
| 3129 | EVQLVQSGAEVKKPGASVKVSCKASGFDIQDTYMHWVRQAPGQGLEW |
| | MGRIEPASGHIKYDPKFQGRATMTRDTSTSTVYMELSSLRSEDTAVYYC |
| | ARSGGLPDWWGQGTTVTVSS |
| 3131 | QVQLVQSGAEVKKPGASVKVSCKASGFDIQDTYMHWVRQAPGQGLEW |
| | MGRIEPASGHIKYDPKFQGRATITTDTSTSTVYMELSSLRSEDTAVYYCA |
| | RSGGLPDWWGQGTTVTVSS |
| 3133 | QVQLVQSGAEVKKPGASVKVSCKASGFDIQDTYMHWVRQAPGQGLEW |
| | MGRIEPASGHIKYDPKFQGRVTMTRDTSTSTAYMELSSLRSEDTAVYYC |
| | ARSGGLPDWWGQGTTVTVSS |
| 3134 | EVQLVQSGAEVKKPGASVKVSCKASGFDIQDTYMHWVRQAPGQGLEW |
| | MGRIEPASGHIKYDPKFQGRVTMTRDTSTSTAYMELSSLRSEDTAVYYC |
| | ARSGGLPDWWGQGTTVTVSS |
| 3136 | QVQLVQSGAEVKKPGASVKVSCKASGFDIQDTYMHWVRQAPGQGLEW |
| | MGRIDPASGHIKYDPKFQGRVTMTRDTSTSTAYMELSSLRSEDTAVYYC |
| | ARSGGLPDWWGQGTTVTVSS |
| 3137 | DVQLVQSGAEVKKPGASVKVSCKASGFDIQDTYMHWVRQAPGQGLEW |
| | MGRIEPASGHIKYDPKFQGRVTMTRDTSTSTVYMELSSLRSEDTAVYYC |
| | ARSGGLPDWWGQGTTVTVSS |
| 3138 | QVQLVQSGAEVKKPGASVKVSCKASGFDIQDTYMHWVKQAPGQGLEWI |
| | GRIDPASGHTKYDPKFQVRATITTDTSTSTAYLELSSLRSEDTAVYYCARS |
| | GGLPDVWGQGTTVTVSS |
| 3139 | QVQLVQSGAEVKKPGASVKVSCKASGFDIQDTYMHWVRQAPGQGLEWI |
| | GRIDPASGHTKYDPKFQVRATITTDTSTSTAYLELSSLRSEDTAVYYCARS |
| | GGLPDVWGQGTTVTVSS |
| 3151 | QVQLVQSGAEVKKPGASVKVSCKASGFDIQDTYMHWVRQAPGQGLEW |
| | MGRIDPASGHIKYDPKFQGRVTMTRDTSTSTVYMELSSLRSEDTAVYYC |
| | ARSGGLPDWWGQGTTVTVSS |
| 3152 | QVQLVQSGAEVKKPGASVKVSCKASGFDIQDTYMHWVRQAPGQGLEWI |
| | GRIEPASGHIKYDPKFQGRVTMTRDTSTSTVYMELSSLRSEDTAVYYCAR |
| | SGGLPDWWGQGTTVTVSS |
| 3153 | QVQLVQSGAEVKKPGASVKVSCKASGFDIQDTYMHWVRQAPGQGLEWI |
| | GRIDPASGHIKYDPKFQGRVTMTRDTSTSTVYMELSSLRSEDTAVYYCAR |
| | SGGLPDWWGQGTTVTVSS |
| 3154 | QVQLVQSGAEVKKPGASVKVSCKASGFDIQDTYMHWVRQAPGQGLEW |
| | MGRIEPASGHVKYDPKFQGRVTMTRDTSTSTVYMELSSLRSEDTAVYYC |
| | ARSGGLPDWWGQGTTVTVSS |
| 3155 | QVQLVQSGAEVKKPGASVKVSCKASGFDIQDTYMHWVRQAPGQGLEW |
| | MGRIEPASGHIKYDPKFQGRVTITRDTSTSTVYMELSSLRSEDTAVYYCA |
| | RSGGLPDWWGQGTTVTVSS |
| 3156 | EVQLVQSGAEVKKPGASVKVSCKASGFDIQDTYMHWVRQAPGQGLEW |
| | MGRIEPASGHIKYDPKFQGRVTMTRDTSTSTVYMELSSLRSEDTAVYYC |
| | ARSGGLPDWWGQGTTVTVSS |
| 3157 | QVQLVQSGAEVKKPGASVKVSCKASGFDIQDTYMHWVRQAPGQGLEW |
| | MGRIEPASGHIKYDPKFQGRVTMTTDTSTSTVYMELSSLRSEDTAVYYC |
| | ARSGGLPDWWGQGTTVTVSS |
| 3158 | QVQLVQSGAEVKKPGASVKVSCKASGFDIQDTYMHWVRQAPGQGLEW |
| | MGRIEPASGHIKYDPKFQGRVTITTDTSTSTVYMELSSLRSEDTAVYYCA |
| | RSGGLPDWWGQGTTVTVSS |
| 3159 | QVQLVQSGAEVKKPGASVKVSCKASGFDIQDTYMHWVRQAPGQGLEW |
| | MGRIEPASGHTKYDPKFQGRVTMTRDTSTSTVYMELSSLRSEDTAVYYC |
| | ARSGGLPDWWGQGTTVTVSS |
| 3160 | QVQLVQSGAEVKKPGASVKVSCKASGFDIQDTYMHWVRQAPGQGLEW |
| | MGRIEPASGHIKYDPKFQGRATITRDTSTSTVYMELSSLRSEDTAVYYCA |
| | RSGGLPDWWGQGTTVTVSS |
| 3161 | QVQLVQSGAEVKKPGASVKVSCKASGFDIQDTYMHWVRQAPGQGLEW |
| | MGRIEPASGHIKYDPKFQGRVTMTRDTSTSTVYLELSSLRSEDTAVYYCA |
| | RSGGLPDWWGQGTTVTVSS |
| 3162 | QVQLVQSGAEVKKPGASVKVSCKASGFDIQDTYMHWVRQAPGQGLEW |
| | MGRIEPASGHIKYDPKFQGRVTMTRDTSTSTAYLELSSLRSEDTAVYYCA |
| | RSGGLPDWWGQGTTVTVSS |
| 3168 | QVQLVQSGAEVKKPGASVKVSCKASGFDIQDTYMHWVRQAPGQGLEW |
| | MGRIDPASGHIKYDPKFQGRATITTDTSTSTVYMELSSLRSEDTAVYYCA |
| | RSGGLPDWWGQGTTVTVSS |
| 3169 | EVQLVQSGAEVKKPGASVKVSCKASGFDIQDTYMHWVRQAPGQGLEW |
| | MGRIEPASGHIKYDPKFQGRATITTDTSTSTVYMELSSLRSEDTAVYYCA |
| | RSGGLPDWWGQGTTVTVSS |
| 3202 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWEGNPRTFGGGT |
| | KLEIK |
| 3204 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWKGNPRTFGGGT |
| | KLEIK |
| 3205 | EIVLTQSPGTLSLSPGERATLSCRASSSVSYMYWYQQKPGQAPRPWIYAT |
| | SNLASGIPDRFSGSGSGTDFTLTISRLEPEDFAVYYCQQWEGNPRTFGGGT |
| | KLEIK |
| 3206 | EIVLTQSPGTLSLSPGERATLSCRASSSVSYMYWYQQKPGQAPRPLIYATS |
| | NLASGIPDRFSGSGSGTDFTLTISRLEPEDFAVYYCQQWKGNPRTFGGGT |
| | KLEIK |
| 3207 | EIVLTQSPGTLSLSPGERATLSCRASSSVSYMYWYQQKPGQAPRLLIYATS |
| | NLASGIPDRFSGSGSGTDFTLTISRLEPEDFAVYYCQQWSRNPRTFGGGTK |
| | LEIK |
| 3208 | EIVLTQSPGTLSASPGERATMSCRASSSVSYMYWYQQKPGQAPRPWIYA |
| | TSNLASGVPDRFSGSGSGTDYTLTISRVEPEDFAVYYCQQWSGNPRTFGG |
| | GTKLEIK |
| 3209 | EIVLTQSPGTLSLSPGERATLSCRASSSVSYMYWYQQKPGQAPRLLIYATS |
| | NLASGVPDRFSGSGSGTDFTLTISRLEPEDFAVYYCQQWEGNPRTFGGGT |
| | KLEIK |
| 3216 | EIVLTQSPGTLSLSPGERATLSCRASSSVSYMYWYQQKPGQAPRLLIYATS |
| | NLASGIPDRFSGSGSGTDYTLTISRLEPEDFAVYYCQQWEGNPRTFGGGT |
| | KLEIK |
| 3217 | EIVLTQSPGTLSLSPGERATLSCRASSSVSYMYWYQQKPGQAPRLLIYATS |
| | NLASGIPDRFSGSGSGTDFTLTISRVEPEDFAVYYCQQWEGNPRTFGGGT |
| | KLEIK |
| 3218 | EIVLTQSPGTLSLSPGERATLSCRASSSVSYMYWYQQKPGQAPRLLIYATS |
| | NLASGIPDRFSGSGSGTDYTLTISRVEPEDFAVYYCQQWEGNPRTFGGGT |
| | KLEIK |
| 3219 | EIVLTQSPGTLSASPGERATLSCRASSSVSYMYWYQQKPGQAPRLLIYAT |
| | SNLASGIPDRFSGSGSGTDFTLTISRLEPEDFAVYYCQQWEGNPRTFGGGT |
| | KLEIK |
| 3220 | EIVLTQSPGTMSLSPGERATLSCRASSSVSYMYWYQQKPGQAPRLLIYAT |
| | SNLASGIPDRFSGSGSGTDFTLTISRLEPEDFAVYYCQQWEGNPRTFGGGT |
| | KLEIK |
| 3319 | RTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVT |
| | KSFNRGEC |
| 3320 | ASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLN |
| | GKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKS |
| | RWQQGNVFSCSVMHEALHNHYTQKSLSLSPGK |

TABLE 19
Fc and Constant Regions

SEQ ID NO: 320 IgG1 Constant
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 321 IgG1 Constant
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 322 IgG1 Constant
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 323 Fc1 (L235E)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 324 Fc2 (L235E)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 325 Fc3 (L235E)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 326 Fc4 (L234A, L235A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 327 Fc5 (L234A, L235A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 328 Fc6 (L234A, L235A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 329 Fc7 (L234A, L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 330 Fc8 (L234A, L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 331 Fc9 (L234A, L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 332 Fc10 (L234A, L235A, P329G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 333 Fc11 (L234A, L235A, P329G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 334 Fc12 (L234A, L235A, P329G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 335 Fc13 (L234F, L235E, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEF
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 336 Fc14 (L234F, L235E, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEF
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 337 Fc15 (L234F, L235E, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEF
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 338 Fc16 (L234A, L235E, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 339 Fc17 (L234A, L235E, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 340 Fc18 (L234A, L235E, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 341 Fc19 (L234A, L235E, G237A, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 342 Fc20 (L234A, L235E, G237A, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 343 Fc21 (L234A, L235E, G237A, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 344 Fc22 (L234A, L235A, P329A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 345 Fc23 (L234A, L235A, P329A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSREEMTKNQVSLTCLVKGFYPSDIA VEWESNGQPENNYKTTPPVLDSDGSFFL
YSKLTVDKSRWQQGNVFSCSVMHEALHNHYTQKSLSLSPGK
SEQ ID NO: 346 Fc24 (L234A, L235A, P329A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 347 Fc25 (D265A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 348 Fc26 (D265A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
LGGPSVFLFPPKPKDTLMISRTPEVTCVVVAVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 349 Fc27 (D265A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 350 Fc28 (N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYGSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 351 Fc29 (N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKP
REEQYGSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 352 Fc30 (N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYGSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 353 Fc31 (D265A, N297A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYASTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 354 Fc32 (D265A, N297A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
LGGPSVFLFPPKPKDTLMISRTPEVTCVVVAVSHEDPEVKFNWYVDGVEVHNAKTKP
REEQYASTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 355 Fc33 (D265A, N297A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYASTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 356 Fc34 (D265A, N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYGSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 357 Fc35 (D265A, N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
LGGPSVFLFPPKPKDTLMISRTPEVTCVVVAVSHEDPEVKFNWYVDGVEVHNAKTKP
REEQYGSTYRVVSVLTVLHQDWLNGKEYKCKVSNKALPAPIEKTISKAKGQPREPQV
YTLPPSREEMTKNQVSLTCLVKGFYPSDIA VEWESNGQPENNYKTTPPVLDSDGSFFL
YSKLTVDKSRWQQGNVFSCSVMHEALHNHYTQKSLSLSPGK
SEQ ID NO: 358 Fc36 (D265A, N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYGSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 359 Fc37 (L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 360 Fc38 (L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEL
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 361 Fc39 (L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 362 Fc40 (IgG4)
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPSCPAPEFLGGP
SVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
SEQ ID NO: 401 (L234A, L235A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 402 (L235E)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
EGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 403 (L234A, L235A, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 404 (L234A, L235E, G237A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 405 (L234A, L235A, P329A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 406 (L234A, L235A, P329G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 407 (P329A)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALAAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 408 (L234E, L235F, P331S)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEE
FGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPASIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 409 (D265A, N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVAVSHEDPEVKFNWYVDGVEVHNAKTKP
REEQYGSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 410 (N297G)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKP
REEQYGSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 411 (S228P)
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
SEQ ID NO: 412 (S228P, L235E)
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGG
PSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK
SEQ ID NO: 413 (S228P, F234A, L235A)
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEAAGG
PSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK
SEQ ID NO: 501 (L234A, L235A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 502 (L235E)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPELEGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 503 (L234A, L235A, G237A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAAGAPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 504 (L234A, L235E, G237A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAEGAPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 505 (L234A, L235A, P329A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALAAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 506 (L234A, L235A, P329G)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALAAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 507 (P329A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALAAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 508 (L234E, L235F, P331S)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPEEFGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPASIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 509 (D265A, N297G)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVAVSHEDPEVKFNWYVDGVEVHN
AKTKPREEQYGSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 510 (N297G)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHN
AKTKPREEQYGSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 511 (S228P)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 512 (S228P, L235E)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 513 (S228P, F234A, L235A)
QVQLVQSGAEVKKPGASVKVSCKASGFDIQDTYMHWVKQRPGQGLEWMGRIDPAS
GHTKYDPKFQVRVTITRDTSTSTVYLELSSLRSEDTAVYYCARSGGLPDVWGQGTTV
TVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 514
EIVLTQSPGTLSLSPGERATLSCRASSSVSYMYWYQQKPGQAPRPLIYATSNLASGIPD
RFSGSGSGTDFTLTISRLEPEDFAVYYCQQWEGNPRTFGGGTKLEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 515 G2 constant domain
ASTKGPSVFPLAPCSRSTSESTAALGCLVKDYFPEPVTVSWNSGALTSGVHTFPAVLQS
SGLYSLSSVVTVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPP
SREEMTKNQVSLTCLVKGFYPSDIAVEWESNGQPENNYKTTPPMLDSDGSFFLYSKLT
VDKSRWQQGNVFSCSVMHEALHNHYTQKSLSLSPGK

TABLE 20
Select Antibodies

| | Heavy Chain | Light Chain |
|---|---|---|
| | CDR SEQ ID NOS | CDR SEQ ID NOS |
| Antibody | (CDR1, CDR2, CDR3) | (CDR1, CDR2, CDR3) |
| A | 1, 2, 6 | 10, 11, 12 |
| B | 1, 3, 8 | 10, 11, 12 |
| C | 1, 4, 8 | 10, 11, 12 |
| D | 1, 2, 6 | 10, 11, 13 |
| E | 1, 2, 6 | 10, 11, 14 |
| F | 1, 5, 8 | 10, 11, 12 |
| G | 1, 5, 8 | 10, 11, 13 |
| H | 1, 3, 8 | 10, 11, 13 |
| A2 | 1, 2, 7 | 10, 11, 12 |
| B2 | 1, 3, 9 | 10, 11, 12 |
| C2 | 1, 4, 9 | 10, 11, 12 |
| D2 | 1, 2, 7 | 10, 11, 13 |
| E2 | 1, 2, 7 | 10, 11, 14 |
| F2 | 1, 5, 9 | 10, 11, 12 |
| G2 | 1, 5, 9 | 10, 11, 13 |
| H2 | 1, 3, 9 | 10, 11, 13 |
| I | 1, 5, 8 | 10, 11, 15 |
| I2 | 1, 5, 9 | 10, 11, 15 |

TABLE 21
Select Antibodies - Variable Regions

| | Heavy Chain | Light Chain |
|---|---|---|
| | Variable Region | Variable Region |
| Antibody | SEQ ID NO | SEQ ID NOS |
| 15 | 108 | 203 |
| 30 | 108 | 204 |
| 64 | 121 | 202 |
| 67 | 122 | 202 |
| 68 | 123 | 202 |
| 75 | 131 | 205 |
| 94 | 124 | 202 |
| 96 | 128 | 205 |
| 100 | 107 | 204 |
| 103 | 120 | 204 |
| 107 | 108 | 202 |
| 109 | 133 | 205 |
| 110 | 125 | 205 |
| 112 | 117 | 205 |
| 113 | 124 | 205 |
| 115 | 122 | 205 |
| 122 | 113 | 204 |
| 125 | 105 | 204 |
| 126 | 114 | 204 |
| 127 | 118 | 204 |
| 129 | 110 | 204 |
| 132 | 128 | 206 |
| 134 | 122 | 206 |
| 138 | 122 | 204 |
| 147 | 117 | 201 |
| 148 | 121 | 201 |
| 149 | 122 | 201 |
| 151 | 124 | 201 |
| 157 | 104 | 204 |
| 158 | 101 | 204 |
| 159 | 119 | 204 |
| 160 | 102 | 204 |
| 169 | 126 | 205 |
| 170 | 135 | 205 |
| 172 | 130 | 205 |
| 173 | 127 | 205 |
| 174 | 132 | 205 |
| 175 | 126 | 201 |
| 178 | 130 | 201 |
| 179 | 127 | 201 |
| 180 | 132 | 201 |
| 181 | 107 | 202 |
| 188 | 115 | 202 |
| 196 | 129 | 205 |
| 197 | 116 | 205 |
| 198 | 134 | 205 |
| 199 | 109 | 201 |
| 200 | 103 | 201 |
| 203 | 116 | 201 |
| 205 | 109 | 202 |
| 211 | 108 | 201 |
| 212 | 107 | 201 |
| 213 | 106 | 201 |
| 214 | 111 | 201 |
| 216 | 112 | 201 |
| 217 | 101 | 201 |
| 218 | 119 | 201 |
| 219 | 104 | 201 |
| 220 | 102 | 201 |
| 221 | 105 | 201 |
| 222 | 114 | 201 |
| 223 | 103 | 202 |
| 500 | 301 | 303 |
| 501 | 302 | 303 |

### 7.17 Example 17: Design of Humanized Anti-TL1A Antibodies with Reduced Cell-Mediated Cytotoxicity

As provided and described herein, Fc variants (e.g. SEQ TD NOs: 401-413) were designed to diminish effector function and subsequently tested for the ability to (i) effectively be purified/manufactured (Table 22), (ii) reduce antibody-dependent cell-mediated cytotoxicity (ADCC), and (iii) reduce complement-dependent cytotoxicity. Test articles tested comprise heavy chain SEQ ID NOs: 501-513, comprising Fc regions that comprises SEQ ID NOs: 401-413, respectively. Heavy chains used were paired with a light chain comprising SEQ ID NO: 514. ELISA titration profiles and EC50s were generated against recombinant TIA antigen (“EC50”, Table 23). Interestingly, Fc mutations did affect purity, as measured by monomer content, for select mutations/Fc variants (Table 22, wild-type IgG1 control).

### Reduction of CDC Activity

Test articles were evaluated for CDC activity, compared to negative control Human IgG4 isotype control, on TL1A-expressing HEK293 target cells. Rituxan (anti-CD20) was used as a positive technical control on CD20-expressing Raji cell. All test articles were used at a final top concentration of 10 μg/mL followed by a five-fold dilution series (7 points total), in addition to a no treatment control, in triplicate. Cells were incubated with test articles for 15 minutes at 37 C, then treated with human complement, at a final concentration of 25%, for 3 hours at 37 C, 5% CO2. Following incubation, cells were washed and resuspended in Propidium Iodide (P.I.) at a final concentration of 5 μg/mL prior to flow cytometry analysis. Total cells were examined by flow cytometry during sample acquisition. Data were plotted on an XY chart, graphing percentage P.I. positive cells against the log of the concentration and fit to a non-linear regression curve. Cell cytotoxicity in the presence of all test articles was not distinguishable from cell cytotoxicity in the presence of isotype control (Table 23). CDC bioactivity was observed on Raji target cells with Rituxan treatment.

### Reduction of CDC Activity

An antibody-dependent cell-mediated cytotoxicity (ADCC) reporter assay was performed for the characterization of test articles and IgG4 Isotype control on HEK 293 TL1A cells. A reporter cell line engineered to express human Fc-gamma-RIIIa V158 (high affinity) served as effector cells.

Prometheus test articles were evaluated with a top concentration of 10 ug/mL (log dilution for 7 points total, in addition to no test article control). Treatment conditions were tested in triplicate, effector and target cells were co-cultured for 6 hours at 37 C with 5% CO2. Raji target cells were used as a positive control, with Rituxan treatment at a top concentration of 10 ug/mL, 7-point log dilution series, and no treatment control. Test article 502 treatment resulted in dose-dependent increase in luciferase reporter gene activity, and 5044 treatment resulted in increase of reporter activity at the highest tested concentration. The rest of the test articles did not induce reporter activity (Table 23).

TABLE 22

| | | Heavy | | | | |
|---|---|---|---|---|---|---|
| | Fc | Chain | | | Purity | |
| | SEQ | SEQ | | | SDS- | SEC- |
| Class | ID NO | ID NO | mg/mL | mg | PAGE | HPLC |
| IgG1, protein | 401 | 501 | 2.65 | 10.60 | 95% | 90% |
| variants | 402 | 502 | 1.15 | 12.65 | 95% | 92% |
| | 403 | 503 | 3.22 | 10.62 | 90% | 89% |
| | 404 | 504 | 1.61 | 11.27 | 95% | 92% |
| | 405 | 505 | 3.43 | 10.29 | 95% | 91% |
| | 406 | 506 | 1.51 | 15.10 | 95% | 93% |
| | 407 | 507 | 2.85 | 11.40 | 95% | 92% |
| | 408 | 508 | 1.55 | 10.85 | 95% | 92% |
| IgG1, glycan | 409 | 509 | 2.33 | 9.32 | 90% | 90% |
| knock-out | 410 | 510 | 1.36 | 12.24 | 95% | 92% |
| IgG4 | 411 | 511 | 1.78 | 19.58 | 95% | 82% |
| | 412 | 512 | 2.33 | 18.64 | 90% | 81% |
| | 413 | 513 | 5.08 | 15.24 | 95% | 90% |
| Control | — | — | 3.70 | 5.55 | 95% | 97% |

TABLE 23

| | Fc SEQ | Heavy Chain | EC50 | | |
|---|---|---|---|---|---|
| Class | ID NO | SEQ ID NO | (nM) | ADCC | CDC |
| IgG1, protein | 401 | 501 | 0.222 | ND | ND |
| variants | 402 | 502 | 0.215 | 100 ng/mL | ND |
| | 403 | 503 | 0.188 | ND | ND |
| | 404 | 504 | 0.220 | 10 ug/mL | ND |
| | 405 | 505 | 0.346 | ND | ND |
| | 406 | 506 | 0.347 | ND | ND |
| | 407 | 507 | 0.329 | ND | ND |
| | 408 | 508 | 0.330 | ND | ND |
| IgG1, glycan | 409 | 509 | 0.340 | ND | ND |
| knock-out | 410 | 510 | 0.293 | ND | ND |
| IgG4 | 411 | 511 | 0.299 | ND | ND |
| | 412 | 512 | 0.324 | ND | ND |
| | 413 | 513 | 0.252 | ND | ND |

### 7.18 Example 18: Biophysical Properties of Anti-Tl1a Antibodies at High Concentrations

The data for A219 anti-TL1A antibody properties in solution were analyzed together using a chemometric method termed partial least squares (PLS). Detailed descriptions of PLS modeling have been published in, for example, Katz, M. H. Multivariate Analysis: A Practice Guide for Clinicians. Cambridge University Press, New York, pp. 158-162 (1999), Stahle, L., Wold, K., Multivariate data analysis and experimental design in biomedical research. Prog. Med. Chem. 1988, 25: 291-338; Wold S. PLS-regression: a basic tool of chemometrics. Chemom. Intell. Lab. Syst. 2001, 58: 109-130; and Martens, H.; Martens, M. Multivariate Analysis of Quality: An Introduction, Wiley and Sons, Chichester, UK (2001).

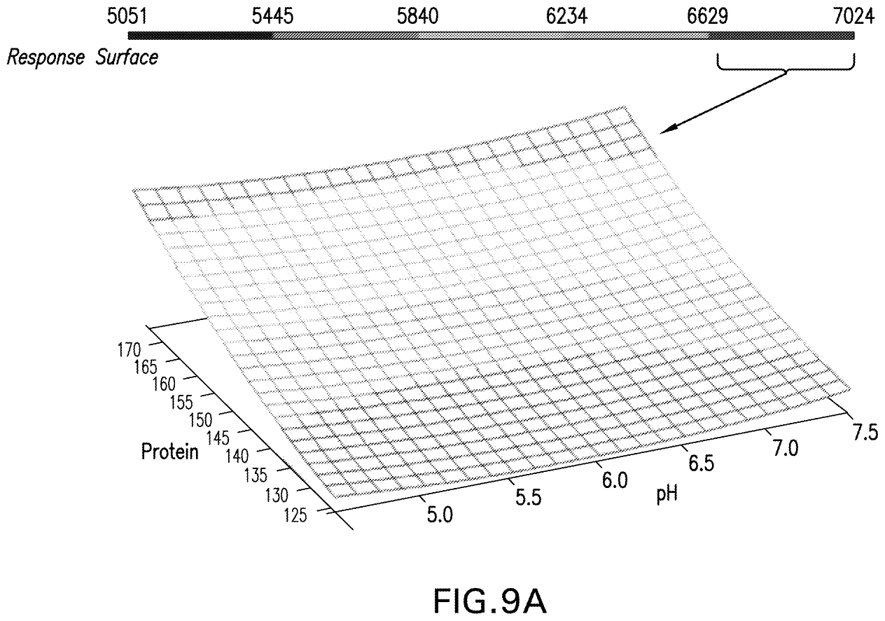

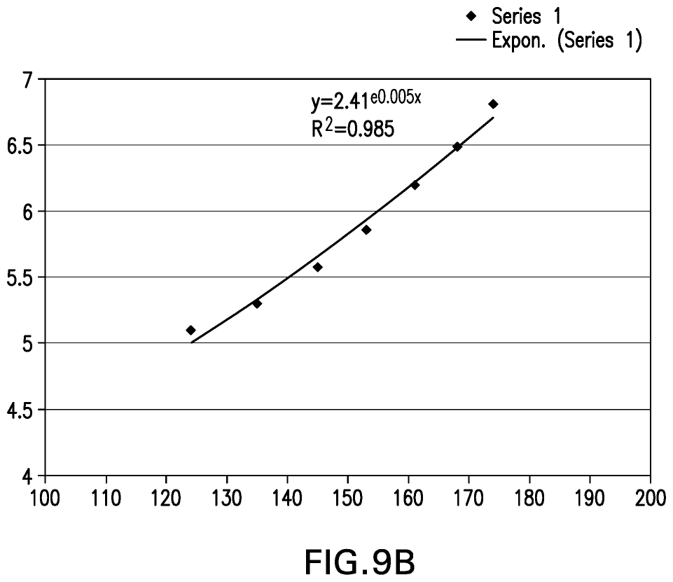

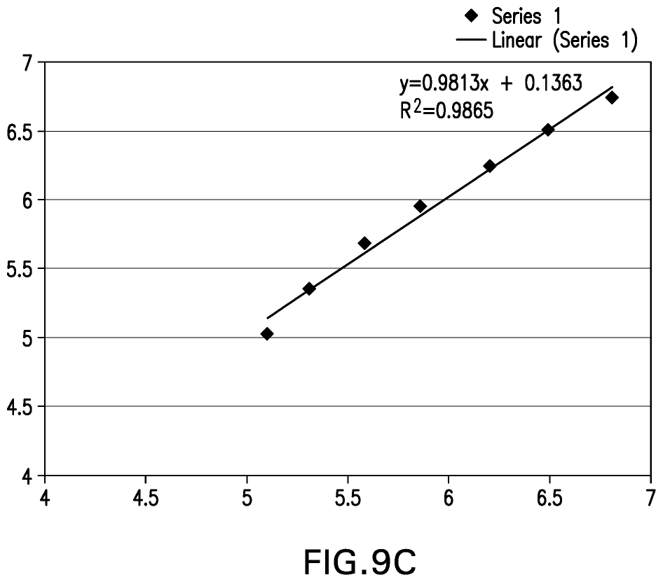

FIGS. 9A-9C demonstrate viscosity as a function of antibody concentration and pH. Antibody concentration ranged from greater than about 125 mg/mL to greater than about 170 mg/mL. pH ranged from less than 5.0 to about 7.5. Concentration dependence is evident, with very low viscosities (e.g. as indicated by a viscosity less than 5 mPa-s or 7 mPa-s). The viscosity was measured using an m-VROC™ viscometer by Rheosense with an A10 chip. The shear rates employed were about 1820 s-1. The viscometer was temperature controlled using a ThermoCube thermoelectric chiller and the samples were delivered using a Hamilton 100 μL syringe (81060). The accuracy of the instrument was verified using neat Isopropyl alcohol and measured at 25° C. Furthermore, across the concentration range tested, the percent increase in the HMW fraction as measured by size exclusion chromatography ranged from 0% to a 1.3% increase. HMW as used herein refers to high molecular weight antibody fraction, e.g., aggregated protein, and which excludes monomeric antibody.

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

TABLE 24
Additional Sequences

| SEQ ID | Sequence |
|---|---|
| 2001 | CATTCTGCAG CAGGGACAGA AGCCTGGGAA GTTGAGTCCA CGAAGAGGGT |
| | TCTCGGACTC CCTTCCCTAA GCGAGTGACT GATGTTTCCT GACTCTGGCC |
| | CTGGTTGTGC CGTGAGGCCA CAGTGAGGGC AGCTGGAGAG CTCAGGATCT |
| | GCCACCTTAG CACACATGGG ATGTTAGGCA CTTCTCGCTA TGCCTCAGTT |
| | TCTGTGGTCT CATGAAGGAA CATTAGTACT TTCTTCATTA ATGCACATAA |
| | AATGCTTGGC AGTGCTTGGC ATAGAGATAG ATGCTCAATA AATGTTGGCC |
| | ACTATTATTA TAGCCCCCTA CAAACAAGGA GGATGAGATT GAAAACAATG |
| | TTTCCTAAAA ATGAGGGTAT AATGGGCATC CTATCCATAT TTCAGGGGTA |
| | GGAAAACAGC CTTCTGAGAT CATTTATATG AAGGGTATGG ATGGACAAAT |
| | CCTTATCAGC CTAAAATATT CCTATCTATG ATTGCTATGT CATTTCTTTA R |
| | GATTAATCAA TCCTCTTCAT GAGTCATATT TCAATATATT ACACATACAC |
| | ATAATTATAA AAGAACCAAG TTGCATAAAA TTTATAAAAT GGAGACTAGG |
| | AAAGGAAAAA CCCGCTCATA AAACCCAACA AGAGAGCTTA AAATGAAAGT |
| | CTGAAAGTGC AGGTAGGTAT TTGTTTAAAT ACTTGGTAAA AGAAAATGTT |
| | CAATTCAGTA GCTGTGACAG ACACAATTTT AAGGGGTGTG GGCATAAGAG |
| | AAACAGAAAG TCCATCTGTC AAAGGGGTAA ATATTTAGTC AAGTCTTAGA |
| | TAGTATTTCT GTAAACAGAA ATTTCAATGG AAAATTTCCA ACCTGAGAGC |
| | AAATAATTTA AGATGGGTTT AATATAAGAA TTAAAGCTCT CGTCTCCATG |
| | TCGCTGCCCA GCCTGAGACC CAGCCCTCTG AGTCCTTGTG GCAGTTCGGC |
| | TTTTTCTTTC TTGTTCCCTC AACTACAACT GGACACTATA ATTTTCTTTC |
| 2002 | GACTATTAAC CAATCATGAA AAACATTTTC AAATAATACT TAATGACAAG |
| | GAAAATGCTC ACAATAATAT GTTAACTATA AAAGCCTGAT AAAAGTAAGA |
| | ATTCAGCATA ATCCCATATT TTAAGGGAAA TAAGTATATG TACAGAAAAA |
| | AGACTGAAAG ACAACAAAAT GTAAAATTGT TTATGAAGAC TGCCTGTGGT |
| | TTATTTAGGT TATCTTTGTT CTTTCTATCT TTTTATATTT TCCAGTAAAA |
| | ATGAGTTTAT ATTGCTGTAG AATCAGGAAA CAAAATATTG TGATGCAAAT |
| | ATGGCTATGG GACTCTTAAG AAAAAGGTTT TTATATTGAT TTGAGTATAT |
| | AACAGAGTAA CATAGCACTA AAGTTCAAAG TACAAGGCCA CCATATGAAT |
| | TTGATTAAGT AAAGGAAAAG AATTATTGAA AACAAGGATG CAAGTAATTA |
| | CTTTTTCATT TGGATTGTTT GTTCAAAATG TTGCTGCCAA GTTATCTCAC R |
| | TTATTTCAGC TAAGCTCAGA GAGACCCCAA CCTCCTGAGT TCAGCATGTC |
| | ATTGGCTGTT CTGTTCCTAC ACATTGCCCT CTTGCATTTT TTCATTTCCC |
| | TGGCTTCAAC TGCCTAGTCC TCCTAACCTT AACTCTTGGG ATTGACCTGC |
| | TGTGTATCTC CTATTTTTCA AAAAAATGCT TTATGCAAGT TTCTAATACC |
| | CACCAAATAC ACTAGTATTG TACTTCATTC GGTACAGTGC AATCAAACAC |
| | AGGTTGAGTA TTCTTTATCT AAAGTGCTTG GGACTGGAGG TGTTGCAGGT |
| | TTGGGAATTT TTTGGATTTT GGAATATATG CATATACATA ATGAGATATC |
| | TTGGGGATGT GACCCAAGTC TAAACATGAA ATTCACTTAT GTTTCACATA |
| | TACCTTATAC ACATAGCCTG AAGGTTAATT TTATACAGTA TTTTAATTTA |
| | ATAAGATATT TAAAATAACT TATGCAATAT TTCAAATGCA TGAAACAAAG |
| 2003 | GAGGTGCATT CCTCGAGAGA AGATTGACAG TTGCTTTTGC AGGTACTGGA |
| | TATATTAGCA ATCCAGAATA TCTTTTTTTG TTTTTGTTTT CTTGTTGTTG |
| | TTGTTTTAAA GACAGGGTCT TGCTCTGTCA TCCAGGCTGG AATGCAGTGG |
| | CACAATCATA GCTCACTGCA ACCATGGATT CCTGAGCTCA AGCAATCCTC |
| | CTGCCTCAGC CTCCTGAGTA GCTGGGACTA GAGCTGCACA CCACCACACC |
| | TAGCTAATTT TTAAAACTTT TTTATAGAGA TGGGGTTGCC CCGGCTGATC |
| | TTGAACTCCT GGGCTCAAGT GTTTCCCCTG CCTCAGCCTC CCAAAGTGCT |
| | GGGATTACAG GCATGAACCA CCACATCCAG TACAGGATGT CTTTAAATTA |
| | TGTTATCAGT TTGGTGTTTC CAGAATTCTT GGAGAGTAGA AATTCTGTCC |
| | CTAAACTTGT GTGATGGTAA AATACAAATT CTTAAACATG TTTTTCTCCC V |
| | TTTACTCAGA GGCAAAATCA AGGTAGACAA GTTTCCTTAC TAACTCGTTA |
| | AGAAAGGCAG GATTTTTTTT TTTTTTTAGT ATTACCCAGT GTGTGACTTT |
| | CAGGGTCTAA GCCTTATGTA AAATAAAAAG GTCTACAGTC AGACTTCCCA |
| | CCTTCATTAA TACCTGAGAA ATTAATGAAA TAGAGAATAC AAATAAAGAA |
| | AAATCTATCA AGTGTGGGCA CTAGATTTTG TCTCCTTTTC TCCTGAACTA |
| | AACCACGTAA ATGGAATCTC CAGGTCCCCA GAGACTGGCA GATATTCCTT |
| | AAGCAAAACC GTTTCAATGT CCTTGCTTAC CTTTCTGAAT TCATATGTTC |
| | CATTATTTGT GGTCTTTAAG GATTTCTCTT TTACTTTTTG CCAGGCCAGC |
| | AATATATTTA ATAATCTGTT GTTTAAAAAA TATTTACACA ATTATAGGTT |
| | TTTTTAAATC AAAAGTCTCA TTATGATTCC TAAGTCTGCC ATATTGCTAA |
| 2004 | TAAGTTCAAT TATAATATGT ATAGGAATAA GGTACCTCAA TATGTAAAAA |
| | ATCATCAAAT GAACTGGGCA GTGAAAATCA ACTGGCTTTA ACTGTTCATC |
| | ATATTTGAGT CATTTCTCTC AAACTCAAAC ATACTAAAAC TTCTTCAGCA |
| | AATCCATCAG TATTTGGTTT TCTTTTATAT GCTGATAAAA TCATAGATAA |
| | TCTTACAATT GACATCCTCC AAAAGATTCA AAGCTGATTC GAACCTTGGA |
| | GTTCAGGTTC CTGGCTTCTC TGTCTACTGG GGGTCAGGAT ACAGGAAGCA |
| | CAAGGAGGAG GGGAGAAGAT TGTGTGGGCA AAGGGGCAGG GAAGGGGCCA |
| | CCACTCACCA CCACTGCAGG ACGAGGCGCT GAGTAAGGGA CGGGGCTGCT |
| | CCCAGCAGAA TCCACAAAGT AGCTCATTCT GCTCTCAGCA CCTGTGTGAG |
| | AGAAGGCGTT GGCTGAGCCT CTGGATGCTC ACAGGTTTGT GGATTAGAAT M |
| | CACAAGGAAG ACACGAACCT TAGAACTAAA AGTCTCCTTA GGAGCCAACC |
| | AGTTCACTTC TTTGCATGGC AGGGAAAGCA ATGGAAATTC AGAAAGATCA |
| | GTGACTAACC CCCAGCACCG GTAAGAGCAG ACCTCAGACA GAACCAGTTC |
| | CCTGGTTGCA AGGTAACATG GATAACAGAC CATGTCTACA CACTGCAGAC |
| | AGCAAGCAGT AAACCAGTGC ACAAGAGGCA CATTTTTGTA GGGTTTCTGA |
| | GCAGGAACCA GTTCTTGTGT GTCTCCCCCA CCCACAACCA GGCCTGGCAG |
| | ATAAGTAGGC ATACAATACA CAGTTGGGGT GTAAATTAAT TGATAAAGGA |
| | ATAAACATAG AAAGGATTGG AGAAAGGAAG GATGGAAAAA GCTCTCCTGA |
| | AGAGGTATGT CACCCAGTAA CCCAAGAAAC AGATAAACAG AGAGAAACCC |
| | TGCAAAAGGG ATTGAGCATT ATTGATCTTG AAAAAAGGAG AAGGGCTATT |
| 2005 | GACCTAGAAG GATGCTTATG AAATGCTAAG TGGAAAAAAA GTGTGTTGCA |
| | ATATGGGTCA TAAGAAATGG TGTCTTTTCT TTTCTAAAGA GAGAATAGGC |
| | TGGGTGCAGT GGCTCACTCC TGTAATCCCT ACAATTTGGG AGGCCAAAGC |
| | AGGAAGATCA CTTGAGGCCA GGAGTTCGAG ACCAACCTGG GCAACATAGT |
| | GAGACCTCTG TATCCATAAA AAATTTTTAA AAATTAGCCA GATGTGGTGG |
| | CACGTGCCTG TAGTCACAGC TACTCTGAGA CTGAGGTGGA AGGATCACTT |
| | GAACCCAAGG AATTTGAGGC TGCAGTGAAC CGTGATCACA CCACTGCACT |
| | TCAACCTGGG TGACAGAGCA AGACCGTGTC TCAAAAAAAA GAGAAAGAAA |
| | AAACTATATA TGTGTGTGTA TCTGCAGAAA TCAGGTTAGA AATACACACA |
| | TTACACACAC ACATACATAT GTGTGTGTAT ATATATATAA TTTATTTAAT R |
| | CTAACGTTAT TGGTAGGTTC TTTAAACATT TTTTGAAACA AATGTGGAAA |
| | TTAAAAAATA ACATGGGCTG GTGGGGAATG TTAATGAGGG GGAGGCTGTG |
| | CACGTGGGGG CAGGGAGAAC ATGGGAACAC TATGTACTTT CCACTCCATT |
| | TTGATGTAAA CCTAAAACTG CTCTAATAAA TACTAAGTTA TTAAAAGCAA |
| | CCGTGAGGCC AGGCGCAGTG GCTGATGCCT GTAATCCCAG CACTTTGGGA |
| | GGCCAAGGCA GGTGCATCAC TTGAGGTCAG GAGTTCCAGA CCATCCTGGC |
| | CAACATAGTG AAACCCCATC TCTACAAAAA ATACAAAAAT TAGCTGGACG |
| | TGGTGGTACA CGCCTGTAAT CCCAGCTCCT CGGGAGGCTG AGGCAGGAGA |
| | ATCGCTTGAA CCCGGAAGGC ACAGGTTGCA GTGAGCCGAG ATCGCGCCAC |
| | GGCACTCCAG CCTGGGTGAC AGAGAGACTC CATTTCAAAA CTAACTAGCT |
| 2006 | ACTGCTAGAT CTGACATACA GAGAAGAGCA ATTACAGGGC TCTTCAAAGA |
| | TGCAGGTGCT GCCTTCACAA ACCTATTTTG CAAGGCATAT ATCAAGGGTA |
| | TTAAATATAT TAGCTGGGCG TGATCAGGCA TGATGGCACA TACCTGTAGT |
| | CCCAGCTACT TGGGAGGCTG AGGCATGAGA ATCGCTTGAA CCCGGGAGAC |
| | AGAGGCTGCA GTGAGCCAAG ATTGCACCAC TGCAATCCAG CCTGGGTGAC |
| | AGAGTGACTC TGTCTCCAAA AAAATAAGTA AATAAATAAA AAGAAGTTCT |
| | TAAGAGAGAA GGAAAATGAT ATAGGTTTGA AACTCAGATC TACATGAACA |
| | ATGGAAGAGC GTTACAGAGA TAACAGGTGA AGGTAAAACA AAACCCAAAG |
| | GGTATAATGG GTTTCCCTTC CCTTCTCTCT TTCTACGAAT GCAGTGAAAG |
| | GTGAGTTGTA ATCATTTCTA ATGATTTGTG CACATTCTTA TAAATATTAA M |
| | TTAATTCATA CACATGGGTA AACATATATA TATTCCTAGA TTATTATTGT |
| | TTATATGACA ATTTTTAATT CTAGTGAAAT AAAGATGTTA ATTTTAATCT |
| | ATAAAACTAC AAATTACCTA AAATTATTTT TGTCCCTTTT CTACATAAAC |
| | TTGTTAGAAA AAAATTATGT GCTCTTCATT TAACAGAAAT TTGAAATCAA |
| | GAGAACTGAT AATGCTCTTA TTTTCCGTAG AAGGTTTTGG ATTGACCAGT |
| | TTGCGTCAGG GAAAATATTT TTGTGCTTTT CACCTTCCTC AAAGCTGACA |
| | GATAGTTAAC TCTCTCCTTT CTTCCTTGGC ATGCAGGTTT CCTCCATGTT |
| | TTAGGTGGGC CTAGAGATGT AAAGTCATGG CCTTTTTCAA AAGCTACAAG |
| | CTGAGCCAAG CATGGTGGCT CATGGCTGTA ATCCCAGCAC TTTTGGAGGC |
| | TGAGGTGAGT GGAACACCTG AGGTTGGGAG TTCCAGACCA GCCTAGGAAA |
| 2007 | ACAAATGGGG AGGGCCTGTG TCCTCTACAA AGGAGTCCCC GTAGAGGAAC |
| | CCCTTCCACA GGGCACCCCT TCCACAGGGA GGGGCTGGGC CAGCAGCCGT |
| | GCGCAGTGGG ATTCCCGTGT CTGGGGGCTT CAGTCTTCTC AGGGGACACA |
| | CGCCCAAGAG GCAGGGAGCT TGGGTGGGGG CTGCACAGCT GTGGGCTTCT |
| | GTTCTGGGCT GCTGCTGCCT AGTGCTGCGG CTTCTGAAGT GTTACCCAGC |
| | CTCAGAGTTC AGCCCTCATC TGTAAAGTAG GTTTAAGAAA ACCGACTCCA |
| | GGTGAAGAAA ATGGAGCAAT ATGCACTGGG CACGGCACCC ATTCCTGTTC |
| | CCAGAGACCG CGGTGGTTCT CACTGTGCGG CATGTGCCTC TCACCTCCCC |
| | TCCCTTCCAA CCACCTTTCG GGTGCTGAGC CGCGTCGAAG GGACTTTTTG |
| | TCTCCTACCT CAGAGGCTCT CCCAAGCCAG CCCTGGCCCC ACTGTCGGGC W |
| | CAGCTCAGCA CGCAGGCGCC GGGACCTTAC CTGTCTGGCT GCCGACAGTC |
| | AGGTTCTGCT GCAGAAGCAC GTTCTCTATG CAGCAGCCAA TGTTCACGCT |
| | GGGGGTCCGT GCGATCCTCA GCACGCTGAC CACGTCATAC AAGCCCCGCA |
| | TGTTCAAGAA GACGGTGTCA TTCTGCAGAG CCTGGTCCAG CAGGCTGTTG |
| | TCCGTCTTAT TGATCCAGTA CACGTTGGGC CTGGGGTAGC CGTTTATGGA |
| | TGTACACGTG AAGGTGAGCT CATCCTGGGA GGGGCTGTGG GGGGCGCTGA |
| | CGACGGGCAC GCTGAAGTTT GCTGCAGGGG AGGGAAACAG ATTGTGAGAG |
| | ATGCCAGACC CTGCTGGTCA AGAAACAGAG GGTACACGGT GCCAAGGCTG |
| | GGTCAGAGGG GAGGCGGCCC ATTGTGCCCC GACATGGGTG ACAGGCCAGG |
| | ACCAGGGCTG TGGTCCGAGC AGCAGGCTCC GCCCTGTCCT GCCCAAGAGT |
| 2008 | CAAAGAAGGT TTTATCTTCT TTTGTGTAAT CCACCAAAGA GATGTCACTG |
| | ACGTTCACCA TTAGCTTGTC CCCTTCTTGC AAGGAGAACA TGGCTCCGAG |
| | GTAGATGGGC TGGAACCAGT TGCTACCTAC TTCGCATACA GACTTGGTCC |
| | CCATGAGGAG CTGGGTTGGC TCAGGGTAGC TGTCTGTTAC CTTGGTGATG |
| | ACCACAGTGA TGGAGTCTGG CTTGTTTGGT CGGCCTGCTT GTCTGATTTC |
| | ACTGCACTCA GAGGTCATCC CACGGAATGT GACCTGGGAG TAAATGAAGT |
| | AGTCTCCCGA CTCTGGGATC AGCAGGAATT TGTTGGTATA GTTCATTCGG |
| | TTCTTGGTGA AGGCCAGGCC TAGTTCATGT TCCCAGTGCA GAGCTGGGAA |
| | CTGATTTTTA AAGTGCTGTG TGGGAGTTTG TCTCACAACT GGAAAGACAA |
| | GAAAGAGGAT TAATTTTCTC ATTGGGAAAC TGTAGACTTT GCTTAAAAAG H |
| | GTCTCATATC ATTTTCAAAA TAGACTAAAG TGATCGAATA TACCTAACAG |
| | CTAAAAACTG CTTTGGGGAG GAATGAATGA AGAATATGTG ACTGGACATA |
| | CACATTTGTT CAAAGAGAAT AACATCTTGG ACTAGTGACC TGGGGCAAAT |
| | TACTTTGCCT TTCTGAGACT GAGTGGTCTG ACCTGAAAAT TTTTAATTTT |
| | ACCTGAGACC TATGATCCTG GAGAACAATG AAGGTTAAGG AATCCCCCTA |
| | TTCTTCTGTG TTTAGGAAAA TGGCTTGCTG CAAGAATTAT CCTTTCCCAA |
| | ATGACTCAGA TAAAACTCAT CGATGCCTCT CTTGTTTACG TATGACAAGG |
| | CCAGGCACCA GACCCTTCAA ATTCCCATTC TTTGCCCCAT AAGTGATTGG |
| | CTGAACTGTT TTATCCCCAT TCATCAATTG GAACAAAACA CTTGTCTTAC |
| | AGGCATGAGG TAAGTGATAG AAACTTGGCA TCTCCCTGGT CCTAATCCTC |
| 2009 | GGTCATGGTG GCTCACGCCT GTAATCCCAG CACTTTGGGA GGCTGAGGAA |
| | GGAGGATCAC TTGAGGTGAG GAGCTTGAGA CCAGCCTGGC CAACATGGTG |
| | AAACCCCATC TCTACTAAAA ATACAAAAAT TAGCCGGGCG TGGTGGTGCA |
| | CACCTGTAAT CCCAGCTACT CGGGAGGGTG AGGCAGGAGA ATTGCTTGAA |
| | CCTGGGAAGT GGTGGTTGCA GTGAGCCGAG ATCTCACCAC TGCACTCCAG |
| | GCTGGGCAAC ACAGCAAGAC TCTGTCTAAA AAAAAAAAAA AAAAGAAAGA |
| | AAAAAAGAGT TATATTATTT ACGACTCTTT TAGCTTCACT TTCTTATAAG |
| | ATTGTTGTGA GAAGTGAGTA AGACACATGC AAAGTGGTAA ACTGCCTGGC |
| | TGTCATAAAA GTCTTGGCTA CTATCACGGC TGGGAGGCAA AGGAGATTCA |
| | GAGAAGGGCA GAATGACTGC ATAAGGTGAT CTGCCTGTAA CTCGTGTTTC Y |
| | TCCTTTCTAG TATAAAGAGA TAAAGGACTT CAAGGTCTTA AACTGGTGAG |
| | GGAGTATTAC AGCCTTTGTA TGGAAAGGTT TGACTTTGTG TCTCTGCTTA |
| | AACCCCCATT GTGTTGCCCC TACCAATTAT TGTGCTGCCC CCTTGTGGTT |
| | AACTGTGCTA AGACCTTTCT TTTGCTCTTC TCCCATTACA TTTCCCACCC |
| | TTCCCCACTC CCTTTTAGAC CCCTCCTCTT AGCTCCCAGT TTCCCAAACT |
| | GTGTGCCAAG GTGCCCAGAG CACCTCAGGC TTGAACTTGT GTTTTGAGTT |
| | GGTTCATGGT TTCAATATTA GATACCTACA CCCGTTTCTA CAATGTCATG |
| | TCTTTGTAAA GATGTATTTT CAGAGGTTGC TCCGATAAAA AGCAACTACT |
| | ACATGAAAAT GAATGTAGAA CAGGAAATGA CAGTGTGGTA TTGTCCAATT |
| | TGATTCTAAG GTTTGATAAG TTCTACAGTG CTCTACACTA AGTTGTAAGG |
| 2010 | TGAGCAGGTG CAGCCCACGC TGTCAGGAAT GCCCTGAGAG CCTGGCAACA |
| | CTCCCGGGAC TCAAGAAGGC AGGTGGGATA AGAGCTACAA GAGAGGTGCT |
| | AATGGTACCA CTGTTATCAT CAAAGAGGGT AGAGTAGATT TCCCCTCACT |
| | GCGCATCTGG GACACCTTAC TGGAGGAGGT GGCATTTGAG CTCGTTTTGA |
| | TGAGATAGCA TATATTTTAA TCAAATCATG TGTACCTGAA TCAGGTAACT |
| | TATATTTTGA TATTATGAAT CAGGTAACTT ATATTTTGAT ATTATGATCC |
| | CTCCAGGAAA ATAAAAACTA GAAGATGGTA ATAGCTCACA TTTATGGAAG |
| | GCGACCTATA CAGCAAGCTT TTCACATCAT GTCCTACGTA TCTCATGAGG |
| | ATGCTGAGAT GCTCAGGGGC TCTGAAACAT ACCCGAGGGT TCACAGTGAG |
| | AGGGTGGCAG AGTTGGAATT TGAATCCTGG TAGGTTTGTT CCCAAAGAGA M |
| | CCTGGCATAT TGGATTATAG GGGCTATTCC TGCCTGACAG ATCGGACAGG |
| | TAGTGGAAGG AGAAATGCAG CTCAGAGCCC CATTTAGGAG GCTGGTTGAC |
| | TGCATCATAA GACACCCTCT GCTGAGGCTT GAAATGCATT GTTCAAAACA |
| | GTTTACAGCC AATTTTGGTT TGCCTCTGAT AGAAGGAGCT GAACACAATT |
| | TTGATTATTT GCTTTAATTC AAATCAGGTT AACAAAATCA GTATATTAGT |
| | GCAATTTAAG GACCTTTTTC CCCCTTTATT ACATAAGCTA GTGGACTTCC |
| | CATCTGATGT ATGAAATGTC ACTTTGTCTT TAATTCTTGA CGTTCACAAA |
| | CACTGGTGCT ACTGGAAAAG GAGTGGGAGT GAGCGTATGT GTGTGTGTGT |
| | GTGTGTGTGC ATGTGCATGT ATGTATATAC ACATATATAT GAGAGTGAAA |
| | ACCAAACTGA GGTTTCAGAT GGACTTTAGA AGGATTGAGC AGGTTTTGAA |
| 2011 | AAAGATCTCC AGATTGCTTG TCCCGCAAAT TCCTAGCATC CTCCTGTACT |
| | ATTGGATGGA CTGTTAGGAA GTTGACTTGT ATTACAAATG CATACTGATA |
| | ATTTTTTTTT AAAGTTGAAA TGTTGATGTG ACTACCTGAA AAGAGATCTT |
| | TGGCTGGGTA CAGTGGCTCA TGCCTGTAGT CCCAGCACTT TGTGGGGTCA |
| | AGGCAGGCAG ATCACTTGAG GTCAGGAGTT CGAGACCAGC CTGGCCAACA |
| | TGGTGAAACC CTGTCTCTAC CTAAAATATT TTTAAAAATT AGCTGAGTGT |
| | TATGGTGCAC ATCTGTAATC CCAGCTACTC AGGAGGCTGA GGCAGGAGAA |
| | TCCCTTGAAC CCAGGAGGGG GAGGCTGCAG TAAGCCGAGA TCGTGCCACA |
| | GTTGCACTCC AGCCTGGGTG ACAGAGCAAG ACTACATCTC AAAAAAAAAA |
| | GAGAGAGAGA TCTTGGGAAT CAGATAGGTG CAATTCCTAC CACCATTAAA K |
| | TCAGAGTAAA ACTGAAGTAT ATTTATTAGT CATTTCTACC TCTTATATAC |
| | AAATATGTCT GGCAGTTGTG ACTTGATTTT GTTTCTTTTC TCCTAAGAAA |
| | CTCTTTTACT CACTCGCTAG TATCATTAAG AGGTATGAAT CAACATCGAA |
| | ATTACCAGAC TTTTAAAATA GCCCTCTTTT TTTAAAAAAA AGTTGACGTT |
| | CTATTTTTGT TTAAATATCC CTTTTGATGT GCTAACTAGG TTAGTCTATC |
| | ACCTTTAATG AAAACTTGAA TTTCTAAAGG TGCATTTCTA AAGCCAAGAA |
| | AACAAGTAGG TTATGACAAA CAAACAAACA GTGGAAGTGA TGCTATGCAT |
| | CAACTGTTCT CCAGAGAAGT ATCAATAAAT TGCTTGTAGT TATACAAATG |
| | CAGATGCATA CATTAGAGAT TTCCAAAATA CAAAATAATT AAGCTTGATG |
| | AAGACTGATA TTATATATGG CAAAGGGAAA TATTTAGGCA GTGTCATCTT |
| 2012 | TTCTTTCTTATTTATTTGTACATTATCTGTTTGTATCCTTGAATTTTCTCTGGGATGTTAT |
| | AAAAGAAAAATATTACAAGCATAATTTCTGACACAATGTTTTATAACTGTTTTCAAAAG |
| | CAATCTATTTCTCTCTTAATTTCTATAGAACCCATTTTAGTCATTCCTATGATGAATGAA |
| | AAAAAACTCACAATTAGTIGTCAGTTTCAGAAAGGAGTCTCAGAAGCATCTTAGAACC |
| | ACCTAGGCGGTCTTATAGGCGTCACAGAAAAATATTCCACCTTACTTATTCTTATCTTCT |
| | TCAAACTGACTCCAAATATAATAAGCAGGCTTAAGTTATATAAGGTTCCTTAAAAGTTG |
| | TTCTCAAAAACAATTAAATGCAGACTGTCCTCAGTAAAACTTGCATTGGCCCAGTCCAT |
| | TTTACCAAGTTTGCTCCTTCTTACATGTACGACTGCCCCTGTGACAAGTTTAAGCCTACG |
| | TTAATTAATCATCTTTCCATAACANGAGACATATTCATCCAACCCTGAGAGGGATAAGG |
| | GGTAACAGGCAAGATATATTCAAATTGTGACATTTAAGGTCAACTTGACTTTAAAACTC |
| | ATATACTGCTCAGTTTTCATATACGTGGCTGAAAACAAGTCATCACAGAGAACTGGTCA |
| | CACTGTTAGAGGACCAAAATAGTAAAATATGAACAATTCTTCCATATTTAATGATATTA |
| | AATATAACAGGTCCTATGTCTATGCAATTGAAGTCAACATGTCACTAATTAGGGCTTCC |
| | TTTGTTATCAAACTTGCTGAGAAGATAGAACAGTGAGGAAAAAATAGCAGGAATTGAG |
| | CTGTACAGAATTAAAGACATTCTTATGAACCTTAAGACTTAAGGTCAGTCACACTCATA |
| | TGACATTTGGACTATTAGACAACTCTTCTGACCTCAAATTTTGCTCTTACCAGTTGGTGA |
| | TAATCCTTTGGTAAGAGAGCATGTTAAAAGTAACCAAATCAGATAAGGACTA |
| 2013 | TTCTGAGCAC TCACATTTTA ATGCTGGGAA TTTTATGCTG AGGTTCAGTG |
| | CTTTTCAGGG AGCCATGAAC CCCCTGCAAT GACTTGCAGA CTGTAGGTAT |
| | TACATGTAAA ACTTGTGTGA GTAATTTCCT GAAGAAGGGG TTTCATTCTT |
| | CAGATCCTCA AAAAGATCTA TGATCCGGCC AGGCGCGGTG GCTCATGCCT |
| | GTAATCCCAG CACTTTGGGA GACCAAGGCG GGCATATCAG GAGGTCAGGA |
| | GATCAATACC ATCCTCGCTA ACACAGTGAA ATCCCATCTC TACTAAAAAT |
| | ACAAAAAATT AGCTGGGTGT GGTGGCGCAT GCCTGTAATC CCAGCTACTC |
| | GAGAGGCCGA GGCAGGACAA CAGCTTGAAC CCAGGAGGCG GAGGTTGCAG |
| | TGAGCCAACA GAGTGAAACT CTGTCTCAAA AAAAAAAAAA ATATATATAT |
| | ATATATATGA TCCTAAAAAG ACATTGATAT TAAAGAGTAA TGATGGGGAA R |
| | GACCACCAAG TGATCATTTA TAAGATGCTG CGAGAATTGC TAAAGTTGAG |
| | GGATGGATTC TGCATTCCAG GAGAACAAAG GAGGTAGATC CCGATTAACT |
| | GAGTAGCAGG ATATTGAGGG AAGCTTCTTG TAATGATTCA GCTGAGTCTT |
| | ACACAATGAA TGAGACTTGG ATAGATGAAC ATGCATTAGG AGACATCAGT |
| | TCTGGTCAGA GGAAACAGCA AATGCAAAGG CCCAGAGCCA TAAGAGATTC |
| | AGATGCACTA AAGAAATATC CCATCATTTA GGATGGTCGG CATATACACA |
| | GAGGGCCTAT AAGGAAGGAG AAGAAACTGA GGTTGTAGAA GCTGGTAGGG |
| | ACTGCATGCT GAAAGGCATT GTAAATCTTA GACTAAAATC TGTGGGAGAC |
| | TTTGAACAAC CTAAAGATTT TAAGCAGAGT GATGCAATGC ATTTGTCTTT |
| | TGAATGCAAA TATTGCTGGG GCGGGTCTTT TGTGTTATCA GCTAATGATT |
| 2014 | TGTAAACTTA TTAGTGCCTC TGCACACCAT ATAATTAAGA TCAAATTCTC |
| | CAAGGGTTTA AAATAGGGAC TGCTGCTATT GTGCTGCTGA CAGCGCTAAG |
| | GAACAGATAT TTGCCTTTTT TACATAGACG GCTCTGCCCC GCTTTTGCTG |
| | AAATTCCCAC TCTCACAGGG CTGGGAGCCC AGCAGGGTTT ACCTGGACCA |
| | ATGAGGGGGG CTCACTCTGC TGGGAAATTT GCTTTTATAG ATGGCACCAC |
| | CAACGCTGAA AACATGGAAG TGGAGGCAAA CATTCCAGAG CATCTACTGT |
| | CGCATCCCCT AGAAGGGGAC TGTGGTCAAG GCAGATCAGT AGACAGAAGG |
| | CTGGAAGGGA AATGAGCCCC AAGGAAGAGG CTCAGATCCA GAGCCTGTGT |
| | CTTGGTGAGA CTCCAACAGA AGTGCCTCGA GGCTGGTGCT TCAGAAGGAA |
| | GTGGGTGTAC AGCTGTGTAA GTGATCTGGG TCATGAATAA ACAAATGAAC M |
| | CAGGAATGCA TGCACCATGA GAGCCTGGAG GGGACTATAA GACAGACAAC |
| | GGGTGATCTG GGCACAGGTA TCAACTGCTG AACGCCCAGT GTGTGCGTGG |
| | AGGAGGAAGG GGGTCAGAAA GGCAAGGAGG ATGTACCAGT ATGGTAGAGA |
| | CGAGCAACTG TGGGCAAGGG GTGCTCAGGA TGCATGGGGG TGTGGAGAGA |
| | AGGGGAGAGC AAGAAAGGCT TCCAGGGGTG AGGAGCCAAG GAGGTCCTGA |
| | AGACAGCTAG GAGTTAGCAG GCAGAGGAAG GGGATGGGCA TCCCAGGGAG |
| | AAGGAACGGC AAGAACAAAG CAGCACAGCC GTGGAGAGCC TGTCAGTGAG |
| | CGGAGAGCCC GTCAGTGAGC AGCTCGCCTG GGTTCCAGGG AACAGGGCCA |
| | GAATTTTTGG CAGGATAGAC TCAAAGGCCT GAGGTGAACC TGCAAACAAA |
| | GGTAAAAACA CTAACTGGTT TTAAGCAGGA GAGAAATATG ATCAGATTT |
| 2015 | TCACTGCAAC CTCCACCTCC CAGGTTCAAG CGATTCTCCT GCCTCGGCCT |
| | CCCGAGTAGC TGGGATTACT GGCATGCACA TCATGCCTGG CAAATTTTTG |
| | TATTTTTAGT AAAGACAGGG TTTCACTATG TTAGCTAGGC TGGTCTTGAA |
| | CTTCTGACCT CAAGTGAACC GCCCACCTCG GCCTCCCAAA GTGCTGGGAT |
| | TACAGGCATG AGCCACCGTG CCTGGCCAGG GGATGATCTC TTTCTAGAAA |
| | TTCATATCCT TCAATTCTGG AAATGTTTCT CATGATAGTT CTTTGATAAC |
| | TGACCCTCCT TCCTTCACTG TATTTCTCTT TCTGGGACTT TTACTAGTCA |
| | GTGTATTTCT GGGTTGGTCT CTCTCTTTTC TCTTTTATTG GCAACCCTGA |
| | TCTAAGGAAC TAAGGTATAG AGGTGGTTGA GTCCTGACCA GAGCCGTGAG |
| | GGTCTAGATT TATGTAAATG ATGGAGCCAT ACACGTTACT TTATTTATAG Y |
| | AAGATATTGT AAAGATTTTG AAATGTCATT TATATGAAAT GTCAAGATTA |
| | TTAAATTCGT AGTTCACTGT AACCTCAAAC TCCTGGGCTC AAGCAATCCT |
| | CCCACCTCAG CCTCCAAAGT AGCTGGGACT ACAGGCACAT CCCACCATGC |
| | CCAGCTAATT TTTGTATTTT TTGTAGAGAC GGGGTTTCAC CATGTTGCCT |
| | AGCCTTGTCT CGAACTCCTG GACTCAAGCA ATCCTCCTGC CTTGACCTCC |
| | CAAAGTGTGG GGATTACAGG CACTGAGCCA CTGCACCTGG TCAATGCATT |
| | TTTAAATATG GTCTAAGGGT ATCAGCAAGT TGCAGATTTG ATAACAGCTG |
| | GGTCTTTTCA TTTAGTAACC ACCCTTTTTT CCTCCTCTGT ACTCTCACAC |
| | AATATTAAAA TACCTCCTAT TAAATCTCTA TTATCCTCTT CTCTTCCTGT |
| | TGCATTTTTC TCCCACAACT TAAGGCTCAA GTCTAAGCAG TGAAAACATA |
| 2016 | ATAAATTAAA ATAACAGATA ACATGGGCTG TTTTTGACAG GAATATATTT |
| | TAGCTTCTGA TAATTAGCTT TAGTAATCAT TAGCTTAGTT AATGAAAAAT |
| | AAAATACTTA TAATATTAAA AATATGAGAA GCTAAACTAT GAGCAACTAA |
| | CAAATCAAAA CCACGTTCTT CTGAATTCCT GACTCCCCCG CAAATAGTTC |
| | CAAATCACTA TAGTATTCAC ATAGCCCTAT AATAACCTTT GTGGATAAAC |
| | AGTGAATTCC ATGTTCAATG TAAAATAACA CAGAAATTTA GAGTTTTAAT |
| | TGCAATGTCC TATGTAAACT TTAAAAGTTT CCAAAGCAAT TTTCATAAAT |
| | GGGATTCCAT TTTCCACAGC CAAAATACTA TACTGACACT GACCCTCATG |
| | AACTGTTACC TATGCATTTG TGTGCCATTT ACTTTTAATT ACAAAATTTC |
| | TCCCCTTGAT TAAATTTATT GTATGCCTTG CAATATATCA AAATGATAGA W |
| | TAAATGTATA AATAAATTAT TATTTATAAC AAAATTGAAG CTTGTCCTTG |
| | GAATCATTTT CTTGTAGTTC CATGTAGCTA AAATGGGGCT TTGATTTCAA |
| | CCTTGCCCCT GATGAGCTGT GTGATGAAAT CTTAGGCAAG TTACTCAACC |
| | TAAGCCTCAG TTTCCTCATC TGCAAATCAG GGAAAATAAT ACATGCTTTA |
| | GACTATTATT TTGAGAATTA AATGAAATCA TGTATACAAA TTGTCAAACA |
| | GGGCACAGCA TACTCAGTAC CTTATCATTG TTTAGTGTTG TTATTTTGTA |
| | TCTACAGTGT TTTTGGAAAA TAGCATGATT AATCAATTGA CTGGAAGACA |
| | CACAGTCTAA CAGACAATAT ATAGACCATT TATATTGGCC ATGAAAGTGT |
| | CCATAATGGC TGACTTAACT GGCTTTAATG TGTAAAATAT GGTGACTCCC |
| | ATGGTTTTTA AGATATGGCT TAAAGTAGAT ATCAATTTCT TTACATTGGT |
| 2017 | GACCTAGAAG GATGCTTATG AAATGCTAAG TGGAAAAAAA GTGTGTTGCA |
| | ATATGGGTCA TAAGAAATGG TGTCTTTTCT TTTCTAAAGA GAGAATAGGC |
| | TGGGTGCAGT GGCTCACTCC TGTAATCCCT ACAATTTGGG AGGCCAAAGC |
| | AGGAAGATCA CTTGAGGCCA GGAGTTCGAG ACCAACCTGG GCAACATAGT |
| | GAGACCTCTG TATCCATAAA AAATTTTTAA AAATTAGCCA GATGTGGTGG |
| | CACGTGCCTG TAGTCACAGC TACTCTGAGA CTGAGGTGGA AGGATCACTT |
| | GAACCCAAGG AATTTGAGGC TGCAGTGAAC CGTGATCACA CCACTGCACT |
| | TCAACCTGGG TGACAGAGCA AGACCGTGTC TCAAAAAAAA GAGAAAGAAA |
| | AAACTATATA TGTGTGTGTA TCTGCAGAAA TCAGGTTAGA AATACACACA |
| | TTACACACAC ACATACATAT GTGTGTGTAT ATATATATAA TTTATTTAAT R |
| | CTAACGTTAT TGGTAGGTTC TTTAAACATT TTTTGAAACA AATGTGGAAA |
| | TTAAAAAATA ACATGGGCTG GTGGGGAATG TTAATGAGGG GGAGGCTGTG |
| | CACGTGGGGG CAGGGAGAAC ATGGGAACAC TATGTACTTT CCACTCCATT |
| | TTGATGTAAA CCTAAAACTG CTCTAATAAA TACTAAGTTA TTAAAAGCAA |
| | CCGTGAGGCC AGGCGCAGTG GCTGATGCCT GTAATCCCAG CACTTTGGGA |
| | GGCCAAGGCA GGTGCATCAC TTGAGGTCAG GAGTTCCAGA CCATCCTGGC |
| | CAACATAGTG AAACCCCATC TCTACAAAAA ATACAAAAAT TAGCTGGACG |
| | TGGTGGTACA CGCCTGTAAT CCCAGCTCCT CGGGAGGCTG AGGCAGGAGA |
| | ATCGCTTGAA CCCGGAAGGC ACAGGTTGCA GTGAGCCGAG ATCGCGCCAC |
| | GGCACTCCAG CCTGGGTGAC AGAGAGACTC CATTTCAAAA CTAACTAGCT |
| 2018 | ACTGCTAGAT CTGACATACA GAGAAGAGCA ATTACAGGGC TCTTCAAAGA |
| | TGCAGGTGCT GCCTTCACAA ACCTATTTTG CAAGGCATAT ATCAAGGGTA |
| | TTAAATATAT TAGCTGGGCG TGATCAGGCA TGATGGCACA TACCTGTAGT |
| | CCCAGCTACT TGGGAGGCTG AGGCATGAGA ATCGCTTGAA CCCGGGAGAC |
| | AGAGGCTGCA GTGAGCCAAG ATTGCACCAC TGCAATCCAG CCTGGGTGAC |
| | AGAGTGACTC TGTCTCCAAA AAAATAAGTA AATAAATAAA AAGAAGTTCT |
| | TAAGAGAGAA GGAAAATGAT ATAGGTTTGA AACTCAGATC TACATGAACA |
| | ATGGAAGAGC GTTACAGAGA TAACAGGTGA AGGTAAAACA AAACCCAAAG |
| | GGTATAATGG GTTTCCCTTC CCTTCTCTCT TTCTACGAAT GCAGTGAAAG |
| | GTGAGTTGTA ATCATTTCTA ATGATTTGTG CACATTCTTA TAAATATTAA M |
| | TTAATTCATA CACATGGGTA AACATATATA TATTCCTAGA TTATTATTGT |
| | TTATATGACA ATTTTTAATT CTAGTGAAAT AAAGATGTTA ATTTTAATCT |
| | ATAAAACTAC AAATTACCTA AAATTATTTT TGTCCCTTTT CTACATAAAC |
| | TTGTTAGAAA AAAATTATGT GCTCTTCATT TAACAGAAAT TTGAAATCAA |
| | GAGAACTGAT AATGCTCTTA TTTTCCGTAG AAGGTTTTGG ATTGACCAGT |
| | TTGCGTCAGG GAAAATATTT TTGTGCTTTT CACCTTCCTC AAAGCTGACA |
| | GATAGTTAAC TCTCTCCTTT CTTCCTTGGC ATGCAGGTTT CCTCCATGTT |
| | TTAGGTGGGC CTAGAGATGT AAAGTCATGG CCTTTTTCAA AAGCTACAAG |
| | CTGAGCCAAG CATGGTGGCT CATGGCTGTA ATCCCAGCAC TTTTGGAGGC |
| | TGAGGTGAGT GGAACACCTG AGGTTGGGAG TTCCAGACCA GCCTAGGAAA |
| 2019 | GGCAATTCCA GTGAAATAAT TGTTTGTTTG TTTGTTGAGA CAGGGTCTCC |
| | TTCTGTCGTC CAGGCTGGAG TTCAGTGGTA TGATCTTGGC CCACTGCAAC |
| | CTCCACCTCC TGGGCTCAAG CCATCCTCCC ACCTCAGCCT CCCGAGTAGC |
| | CGGGACTACA GGTGCACACC ACCACGCCCG GCTAATTTTT GTATTTTTTG |
| | TAGAGGCGGG GTTTCCCAGC GTTGCCCAGG CTGGTCTTGA ACCCCTGAGC |
| | TCAAGTGATC TGCCCACCTT GGCCTCCCAA AGTGCTGGGA TTACAGGTGT |
| | GAGCCACCGC GCCCGGCCTG AAACAATCGT TTCTAAATAT TGGTGTGGGC |
| | CACACAGTCA TGTTTGGACC TACTTGTGGC CTTTTACAGA CCCCAGGCCA |
| | AGGCTTTGGG AACTTGGCTG TCAGCCTCCT GTGCCTTCTG CACCCCCACC |
| | CCATTTCTGC TTTCTGGAAC CCCCGATCCT GTCCTGTTCT GTGGTGATTC R |
| | GGTGTGCTTG GGCTCTAGGA GAAGATGTGT GAAGAATCGG CATCCTTTGA |
| | CCTGACTCCC CATGACCTGG CTTCAGGACT GGACGTCATA GACCAGGTGC |
| | TGGAGGAGCA GACCAAGGCA GCGCAGCAGG GTGAGCCCCA CCCGGAGTTC |
| | AGCGCGGACT CCCCCAGCCC AGGTGCGTTC ATAGCCAGAC TGCTTGGTCC |
| | TGAGGCCTGC GCTGCTGCAG GGTGAGCCCC ACCCGGAGTT CAGCACGGAC |
| | TCCCCCAGCC CAGGTGCGTT CATAGCCAGG CTGCTTGGTC CTGAGGCCCG |
| | TGCTACTGCA GTGGGCAGCC TGCCCTGTGG CTGTGTGTGG TCGGCCTGGG |
| | CACCATCTAT TCAGGCTGGC ACTGCAGGGC ATCCGCTTCT CTCAGAGGCT |
| | TCTTGGGTGT GAATTCTTCA GGGTCCTGTA GCCTGTGGAA GGGCTGGTAT |
| | TGTTCAGTAG TTCTGGTATT TTCCAAAGAC CTATGTCTTC TCCCAGCCAG |
| 2020 | GTTGGTGGAT TTGGCCTGCA CGGATTCTGT GTGGCCTCTT CCTTCCCCTG |
| | TTGGTGGATT TGGCCTGCAC GGATTCTGTG TGGCCTCTTC CTTCCCCTGT |
| | TGGTGGATTT GGCCTGCACG GATTCTGTGT GGCCTCTTCC TTCCCCTGTT |
| | GGTGGATTTG GCCTGCACGG ATTCTGTGTG GCCTCTTCCT TCCCCTGTTG |
| | GTGGATTTGG CCTGCACGGA TTCTGTGTGG CCTCTTCCTT CCCCTGTTGG |
| | TGGATTTGGC CTGCACGGAT TCTGTGTGGC CTCTTCCTTC CCATGTTGGT |
| | GGATTTGGCC TGCATGGATT CTGTGTGGCC TCTTCCTTTC CATGTTGGTG |
| | TCCTTTTTTC CATGCCAGGA ATCCTGGTTC TCAAGGGCGG GGTTGTTGGC |
| | ACGAGCGTGA TGCAGACTGC CTTTGCTGCC TTTCTCTTGC CCAGGGCTGA |
| | ACATGGAGCT GGAAGACATT GCAAAGCTGA AGAGTAAGTG TTGCCCTCCC Y |
| | GCCTCCTTGC AGCTGGGTGG GGCCTCCTCC TTGCGAGGAG GTGGGTGACA |
| | CCTCCTCGAC CCACAGTGAT CCTGCTGCGC CTGGAGGGGG CCATCGATGC |
| | TGTTGAGCTG CCTGGAGACG ACAGCGGTGT CACCAAGCCA GGGAGGTGAG |
| | AGGCGGGGAG CCAGCCCCTT CACTGCAGGC CCAGCCTAGA GCTAGAAACG |
| | GGCCATGGTG CAGTCCTGGG CTGTCACATC ACGAGTGAGG CCTGTTTTCA |
| | GGCCTGTTTT CCCTTTTTGA GACCTGGGAG GAGCACCTGC TTTGCATGAT |
| | CTGGTTGCTG AGATGTTGAG AGGAGCAGCA CACACTCCCA CGGGACAGCA |
| | CACAGCCCCC CACGGAACGG CACACACACC CATGGAACAG CACACACACT |
| | CCCACGAACA GCACACACAC TCCCACGAAC AGCACACACA CTCCCACGGA |
| | ACAGCACACA CACCCACGGA ACGGCACACA CACCCACGGA ACAGCACACA |
| 2021 | AAAAAAAATC CACATCCTAA CTCCTGGAAC CTATGAATAT GTTAGATTAC |
| | ATGGCAAAAA GGACTTAAGG CTGTGGATGG CATTAAGGTT GCTAATTAGT |
| | TGCCCTTAAA GTAGGGAGAC TATCCTGGAC TGTGGAAGTG AGCCCTGGAT |
| | TATGAATGTG ATAGGGAAGC AGAAGCATAG AGAAACTATG TTACCAGGTT |
| | TGAAGGTGCA GGAAGGAGCT ATTAACCAAG GAATATGGGC AGCTCTAGAA |
| | GCTGGAAAAG CAATGGAATA ATTCCTTCCT AGAGCCTCCA GAAAGGCACA |
| | CAGCCCTGCC ACACCTTGAT CTTAGACCAA GGAAACCTGT GTCAGACTTC |
| | TATCCTACAA AACTGTAAGA TAGAATTGCG GGTTGTTTAG GGTGCTATTT |
| | AGGGTAACTT GTGACAGCAG CAACAGAAAA GTAATGCACT TTCTCAACTT |
| | TCCTCTCTCC CTTCTCCCCC AGAACCAAAC ACTGCTGGCT CTGGGCTCTC R |
| | CAGAACACTG TCCTTACAGT ATTTAATGCT CTTGATACTA TCTGGGGCCT |
| | GGCCACGTGC ATCCCATCCT TCCCTTAGCT TCTCCAGGAG CATTGTGGCT |
| | TGGTGTCACG ACCTATCTGA AAGCCTTAAC CCTGCCACCA CCCACTTTCT |
| | GCTGGAAGGA CAGGCACTGT GAAGTGAAAG GACCCACAGC CCATGAACTG |
| | GAAGGAACAG GAAGGGCAGG AGAAAGAAGA AATAAGAGGA GAGCAGCAGG |
| | GGGATGCAGG GAGGAGGAGG ATAAAGGGGT TACCAGAGGA GAGGAGGTGA |
| | GAAAACAGAG GGAAGGAATG AAGGAGGAAG ACAGGGTCAA AGAAGAGGAG |
| | AAGGAAGGAA GGGCGCTGGA GGAGGACACA GAAGCTAGGG TGGCAACGAA |
| | GAGGAGAAGG GAGAATGAAG AGAAAGGACC TTGTTCTGCC CCCACCCATC |
| | TCCCACCTGA ACACCCTCTC CTCCCTCCTC CCTGAGATCT CCCCCAGCCT |
| 2022 | AGGAAATTTC ATAGAAAAGG AAGGGAAACA GTGACACTCT CCAGTTTTGG |
| | CCTGAGCATA TAAAATTATT TGCCTCTGAA GCCCTGAGAT CCTAGGCCAA |
| | AATAAAACAA AACAAATAAA ATTAAACATC ACGATAACTT CAAAGCCTTC |
| | GTTTCTACAT AAGCAGGAAC TGAGGAGGAA ACTGAAGCAT TGGGCTGCCT |
| | AGGTATATGA TCATTGCAAT GTCTCTAGGT GGATGGATGA GTGCCTGGGG |
| | AGCAGCATAG ACTGATCTCT TGCTTCTACT TCTAAATCAT GGACTTCCCC |
| | TATCATCTAC CTAACAATGT TGGACCCTGC AATTTCAACC TGTGAATTTT |
| | GTGATACTTA TTATATGCAG TTTTCAAGCT CTCTTTAGTT TGCTTTTTCT |
| | TAGGTGGAGA AAAGACTCAT TCAAGTTGAT GTTGATAGTG GATATCACCC |
| | CATCAGATAC TATGATCATA AGATTAGAGC TTGGAGAGCC CCAAAGAGTA Y |
| | CTGGTCTTCT TTTTCAAGCT GAGGATACAG AAAGCCCAGA AAAAAACATG |
| | ATTTGCTGAA GGTTACCTAC ATAGTGAGTA AATGCATGAG AATGAGAAGC |
| | CAGGTATTTT GCTTTCTGTG ATCACTCTTA TCAGAGGAAA TTCTTATGGT |
| | TGCACTTATG ACAGCTGCAT TAAGAGTCTG TACAATGGAA AGCTTTGGAG |
| | GAGCTGTTAA ACATATGGAG TCTAAGGAAA TGAGAGTCAG AGAGACGGCT |
| | TCAACTTATC CCTTATCTAT AACTTGGCAG TAGAGCTTTC AAGAGCTCCA |
| | ACCGCCAAAG CTATGGTTTG CTTTCTGAAT TGCTGAAACC ACAGTGAAAA |
| | AGGAGAAATA ACAGGAGTGT ACAGATTTTT CTGTGTTCTT TGGCTTTCAC |
| | ATTTATCTGG TCCAATAAAT TCAACAGGTA TTTCACTGCA AGTTGAAGTT |
| | GAATGATAAT GTACAACTAT GAGGTATATT TTCTCAGCCA TTTCTCTGAA |
| 2023 | CTCCACTGTA GCTCAAGTGT CAATGCTTAA TACTGCTTGA TTTTCCTGAA |
| | TGATTTCTCT ATCCCCCAAA GTAAAATGTG TAATTTCCCT TTTTCCGACT |
| | CCAATGTTCC TGCACTTACT GCCAACATAC CACTTTAAAA TACTCTAACA |
| | TCTGATTTTT TTATTTCCCA TTACATATCT ATTACATATC TCTTGAAGAC |
| | AAGAAATCAT TTGTATTTTT ATATCTTGTT TTCAATATTT AAATGACACA |
| | TGCTATTTCC ATTCCCAAAT TAATTGATAA ATACATGAAT AAATAAAATA |
| | GCTGTGCCAT ATAGTTATCC TTGATATCAC TCTAGATGAG GAAACTGAGG |
| | CTGGAAAGAT TAAACAGCTT AAGTAATATT AACAAAGCAA ATAACTCTTG |
| | GGACCTCCTT GCTCTGTGTA TGTCAAAGCT CATGCTCTCT TTCAACTCCA |
| | TTACATCTCT TTGAATCAGA TAGTAAGATG AAACCTCTTC TAGGAAGTAA R |
| | GACTTCCTGG CCCCAATCAA TTCTTTTTAC TTGCCACCTG CTTGTGAATA |
| | TTAATATTGC TTTCGCTGGA TTTTAAAAAG TAGACTCCTA GGTAAACAGC |
| | AAACAAGAGA GGCAGAGGCC AGAATGCTCA GAAAAAAGGA TACAAACTGG |
| | CGCAATAAGG ATTATATGGT CTGAAGCAGA GGTATGTGGA AGCTGAGGTG |
| | GGAAAGTGAT GGTAAAGCAA ACCAGACCCA AGGATATTGG TTTATCTTAT |
| | GGCAATACTT AACCCATGGA ATGAGAACAT TTGTATGAAT TATCCATGCC |
| | ATTGTTTTCC TCCATATAAG ATATCATATT TCCCATACGT GGTTTGGAGA |
| | GACTTCCTGG AGTAGATCGA TTCCAAGTGT ATCTTGAATG GTAGGGAAAA |
| | TATGGCTTAG TGTTTGGAAA AAGGGATCTA ATTTGCAGAC TTCAAGGCCA |
| | GTGAGATTGA AGGAGCTGAT TCAAATAAAA AATAGGCATA CTTGAAGCAA |
| 2024 | TTAGCTTCTG CTTGAGCATT TCTACTCACT GGAGTTCATT ACTTTCTGAA |
| | ACTGTTTCCT GTTGAACAGC TTTAATGGTT AGAAATTTCT TTGTTATACT |
| | GATCCCAGGC ATGCATCACG GTGATTTCTA ATAATTGACA AAATTATTCC |
| | CCAGTTTTCT TAATCTCTTC TCTACCTGAC CTGGTTCATA GTAATATATT |
| | TATATTCCTC AATATGCATT CTACTATAGC AATATTTATT TTTTCATTCT |
| | CATTGGAGAA TTGTGGATAA TGTATTGACT TCAGAAAATA TACATGTGTA |
| | ATCAGTATAC AGGTAGACTA CTGTGAGGGC CACCTTAGAG GGCTGTTTTT |
| | GGAAGCTCAG GATCCAATAA ATCTAACACC CAAGACCCAT GATTAGCTCT |
| | TCATGGGTAG TTGAGGTCTT TCAGGGGGGT TTATTTTTGG TTAGTTACCT |
| | ATCTTAATCT TTTTGGTTCA TATCTCTTAT GGGGAGAGAG GTAGGGAGGG R |
| | CATGGAAAAT ATCCTATCAA ACCTCCATTT ATAATTGTGC TCACTATCTT |
| | ATAAATAAGA CAAGTAAATT TTTCTAAAAC TTCGGAAGTT TTCTTGATAA |
| | TTATATAGTC ATTTAAGGAT TAATGAATTC TCTAAAGTTT AGCCAGTTAT |
| | CCATATTAAT GGTAGCTAAA AATTGAGAAT GCCACAAAAA TTTCAGACAT |
| | CTGACTTTTT AAAAATTAGA ATTTTTTGGC AGGGTGTGGT GGCCTGTAAT |
| | CCCAGCACCT TGGGAGGCCG AGGCAGGCAG ATCACTTGAA GTCAGGAGTT |
| | CCAGACCAGC CTGGCCAACA TGGTGAAACT TTGCTCTACT AAAAAATACA |
| | AAAATTAGCT GGGCCTGATG GTGCACACTT ATAATCCCAG CTACTTGGGA |
| | GGCTGAGGCA GGAGAATTGC TTCAATTTGG GAGGCAGAGG TTGCAGTGAG |
| | CCAAGATGGT GCTACTGCAC TCCAGCCTGG GTGACTTGTG ACTGGGCAAC |
| 2025 | TTTTCCATCAGGCTCAGAAAGCAGAGTGACATAAAAGCTGGGTATGTGCTAAGGAGGA |
| | GGGGGAGGGAAAGTCTCCAAATAAATCATTTAACTCACAGAGTTTAAGACTGGAAGAT |
| | TCTCCTGGGCCTTGTAAGCTATAAAAAAAAGCAGATCTCTGTCAAGAGGCCTTTTCTCT |
| | AAAGCAGTTCTGACTCAGCCTATTTGCCCTTGGAA |
| | GATGCTGCAGCACCTAAGACATCCACAGGCCATCAGTCAACACGTGATCTCCTCTCTAG |
| | CTATGCTCCCTCTAGAAATGCAGGATCTTTTGCCCCATCCTAAACTCAGAAAAGGGGTT |
| | TCATGTGCAGAACATGGTGGAGAATCCTAGGTCTCTGACTCACAAAAGGATCACCTTGG |
| | GCCTGAGCTTCCTCATCTATGAAATGGGGATGG |
| | GAATTCATGCCTTGCTGTATAATTGCAAGGATTCTATGGAGTAATAAAGGTAAGCGTCT |
| | GGCATGGTTTCTGTACATCTCSGGTGCTCCTGGTACTGGCTGTCACACAGGTCAATAAA |
| | TATCATTTCCTCCTAATCTTTCCACATGCATAGTTATTGGAAAGCTGGGTGGGCAGCAG |
| | GCTATAATTTTTATTTTGCCCAAAAAGGCAGGACCTTATTTTGTTTATAGAAGGCAGAA |
| | CCTCTTTGTTCTGCTTGAAACAAACAAACAAACAAAAAACCTCTCCAAAGGCTCTTTTC |
| | ATCCTCTGCTAAATTGGACAATCCAGTCGGTCCCTCTGGAGTGGACTCAGTTGATTGCC |
| | TACCCAGCACCCATTTCTCCCCAACTCCCTACCTCCTTTTTATTTCTCCTGCTTTATAGAA |
| | CTCTGATTTCATACAGATATATTCATGTTCCTCCCTGGCCATGTGCCTCAAGGAAGGTA |
| | ATCCCACTCCAGCTCCAGGGGGTTTTCTGATTGGTCTAAGACTTGGTTCTCAATCTCAGA |
| | GCTATTGATATTTTGGGTTGGATTTTTTTTTATTTTTTATTTTTATT |
| 2026 | CTATCTGGTGGAATTTGAAATATTTGTGTGATCCAGGCACCTATCTTTAAAAAGGATGG |
| | AGTACTTGACCTTCCATCCTTGTCCCATGGCCCTTCTTCCTTTGCTTGTTCAGTCAAGCC |
| | AAATCCCACCACGTCTCTATCATTGTAAAACTTTTCAGGGAGAAAACCTCAATGGAACC |
| | AGGGAAAGCATACCTACCAGGTCTTTTACTTTTACTTTTTTTTTTTAAGGTTGAGTTCCA |
| | CACACTGTTGGTGGCACCTAAAAAACCACTGCCACCAGTTGAAAAGTTAATTATGGATT |
| | ACCCCTTGAAATAAAATACAAGTTTTATTAGAAGTGGAGGGATAAATCAAAACAGCTG |
| | GAGGGGGAATGCCATGAAACTGCTATGACGAAGCCCATGATGTTCTACTTAGAGTAAA |
| | CATTCTTTTTATTTTATTTTTTTTAAGTCCTGGGTCTGCTGTGTAGAAAGTCATCATCGCT |
| | TTCATGTGGGACAAAAGAGGATTTGGNAAGGGAATTTCCTTAAATCTGTGCATTGAAA |
| | ATGCAGCTTATGGGAGCCTAAAGAAAATACGTTTTGCTTTTTCTTACAGTGGGTGCCTC |
| | TTAATTGTGACTTTTATAAACCCCTTCTGTAGCTATCATAACCATCCAATTATCTAACTT |
| | CATTTTCGTATGATTAAAAGAGCCTCTCTTGCCGAACATATGACAATTATTAGAACGTT |
| | TCTAAAATCTTTTTTTCTAGTGATATGAAACTCCTGTAGATAAAATAAGTCTTGATTTAT |
| | ACAATCCTCAAGTACATGTGAATGTTATGTCTATAAATAAATACTCCATAGTAGATGCA |
| | AAATCCCTTTATAAGTAACAATACATTGGCATATTCATTGACCAAAAAGCCTTGTGATG |
| | TTAATGTTAACAAAATTAAACTTCAGCCATTTCATTCAACTAGTAATTCCTGAGTTCTAA |
| | GGACTAGGCACTATGCTAGGCAGGGAAGCTATATATGTATGTGTGTCTGTGCC |
| 2027 | ACCATCCATGGTGAGCGGCTCTAACTTTCAATCTGGCCAGATCATTTTCCCCTTGTATTA |
| | AAATTGTCCTGGAAAACCATTAGGTTGATTTTCAATCATCACAGCTAATACATTCCTTGT |
| | TGCAAATGCCAAGTATTCTGCACTTGCTTCAGTTTTTCAGATCTTCTCTCTCATATTCAG |
| | TACCTGGGGTGAAATCCATGCCTAGTAAAACAGCCCTTTTGTATCCAGGCCAGGATGCA |
| | TCTCTGAGACTTGAATGCTTTTGCTTTCTCTCCTGTTTTTTGTTTTGTTTTGTTTTGTTTTG |
| | TTTTGGTTTTGTTTTGTTTTGTTTCTCTTCTCTGCCTGATCAATACTTTCCCATCTGAAGA |
| | AGCAAAACATTCTCTCTCTTCTGTTTCCCTTGCTGCTGATTCTGGCTCAAGGGGATGGGG |
| | GCAGGAGTGGAGGAAAGAGGGTCAGAGTCTATATTTAACCACAAAGATAATTCCTCCT |
| | TGACAGCAGCTCCAGAGAGANTTTGACATTTGAGGGGACTGAGTAGCAAGAGTAATTT |
| | GCATTTATCTGGCTATAGAAACAGAAGAGATTGGGAGCAACAAGTGATCTGTGGAGGT |
| | AAAAATGAGTCAGGGGTTTGGAAGAGGGAATATAGGGCAGGTGAAGAAGAAGAAATT |
| | ATTTGCCAACAATGATGGAAGGATAAAGAACAAGACTCCTGAAACTATTTCCTTAAAT |
| | GTCACACTAAAGCAACTCCAAGAGGATTGTAATAGCTTTGGAGTGGGACAAGTGGCAC |
| | AGATGGGAAGGCTGACATGTCCCGGCTGCTTAAATACTCTGAATTCTAGATCTGCATTT |
| | ATCAAAATTTAATGTACACCCGAATTGCCCTGGATCTTGAATAAAATGCATATATTAAT |
| | TCAGTAGGTTGGGGTGGGCCCCAAGATTTTGTATTTTTAGCAAGCTTCCAAATGAAGCT |
| | GATGTTGGTCCTGGGACCACACTTTGATTAGCAAAGCTCTGCTAGAGCGTTTTGG |
| 2028 | ATATTCTTTC CTGTCTGCTG CCTTGTAAGA CGTGACTTTC GCTTTCTGCC |
| | ATGATTGTGA GGCCTCCGTA GCCATGTGGA ACTGTGAGTC CATTAAACTT |
| | CTTTTTGTTT ATAAATTACT CAGTCTCATG TATGTCTTTA TCAGCAGCAT |
| | AAAAACAGAC TAACATAACC CCCCTTTCTT CCTACCCCTG TGATTTGGGT |
| | GGATATTAAA ATTTTGCAAG CCGCCACTCT AGAGAAGTAC CACTCAAACC |
| | ATGGTCTGCA GACTACCAGC ATTAGCAAGG CCTGGGAGTT TGTGAGAGAT |
| | GCAGATTCTT GGTGCCCATC CAAACCTACA GAAGCAGAAT CTCTGGTGGC |
| | AGGAACCAGC AATCTGTGCT TTCAACAAGC TCTCTGAGTG CTTCTTCTGA |
| | ATGTTAAAGT ATAAGAACCT CTGCTGCACA GGGAGACCTC ACTTTCATTA |
| | TTCTCATTTC ACAGATGAGA AAAATGAGTC AGTAGGAGGA AAAGTGATTG N |
| | CTCAAGTGCT TAATAAGTGT CTAAGCTAGG ATTTGACCCT AGGGCTTGTG |
| | AATCTGGAGC CCATAGACTC AACTCCTGTG CTATACTGCC TCTGTGTCTT |
| | TGGTCTGTGT TCTCTTCTTT TTTTTTTTTT CTTACCACCC TCCAAAATTC TTATTTATTT |
| | ATTTTCTATC ACCGTACTTA CTGGTGGAGT GGAAGTCCCT TGGAAGTGGG |
| | GGACGTGTCT GATTTGTGCA TGGCTATTTT GCGATTTGCC ATTGCTGGAA |
| | TATTTAGCAA GCATCAATGA GTCCTGGAGG GCTTTTAAAC CATAGATGAT |
| | CAGGGCCCCT GCTTGGTCTA GGATGGAGCC TGCAATTTTG CATTTGTGAC |
| | AAGCTCCCAC GGATGCTGGT GCTCACTTGG CATAGCAAGG GGTTATGCTA |
| | TTTTTCAAAG GCAAGAGAAG TGTGCATGTA CTTGAGAGGA GAAGGGGTCG |
| | GTCACCATTT ACTTTGTGCC TCTTGCTACC CTTCGTTGCC |
| 2029 | TAAGTTCAAT TATAATATGT ATAGGAATAA GGTACCTCAA TATGTAAAAA |
| | ATCATCAAAT GAACTGGGCA GTGAAAATCA ACTGGCTTTA ACTGTTCATC |
| | ATATTTGAGT CATTTCTCTC AAACTCAAAC ATACTAAAAC TTCTTCAGCA |
| | AATCCATCAG TATTTGGTTT TCTTTTATAT GCTGATAAAA TCATAGATAA |
| | TCTTACAATT GACATCCTCC AAAAGATTCA AAGCTGATTC GAACCTTGGA |
| | GTTCAGGTTC CTGGCTTCTC TGTCTACTGG GGGTCAGGAT ACAGGAAGCA |
| | CAAGGAGGAG GGGAGAAGAT TGTGTGGGCA AAGGGGCAGG GAAGGGGCCA |
| | CCACTCACCA CCACTGCAGG ACGAGGCGCT GAGTAAGGGA CGGGGCTGCT |
| | CCCAGCAGAA TCCACAAAGT AGCTCATTCT GCTCTCAGCA CCTGTGTGAG |
| | AGAAGGCGTT GGCTGAGCCT CTGGATGCTC ACAGGTTTGT GGATTAGAAT M |
| | CACAAGGAAG ACACGAACCT TAGAACTAAA AGTCTCCTTA GGAGCCAACC |
| | AGTTCACTTC TTTGCATGGC AGGGAAAGCA ATGGAAATTC AGAAAGATCA |
| | GTGACTAACC CCCAGCACCG GTAAGAGCAG ACCTCAGACA GAACCAGTTC |
| | CCTGGTTGCA AGGTAACATG GATAACAGAC CATGTCTACA CACTGCAGAC |
| | AGCAAGCAGT AAACCAGTGC ACAAGAGGCA CATTTTTGTA GGGTTTCTGA |
| | GCAGGAACCA GTTCTTGTGT GTCTCCCCCA CCCACAACCA GGCCTGGCAG |
| | ATAAGTAGGC ATACAATACA CAGTTGGGGT GTAAATTAAT TGATAAAGGA |
| | ATAAACATAG AAAGGATTGG AGAAAGGAAG GATGGAAAAA GCTCTCCTGA |
| | AGAGGTATGT CACCCAGTAA CCCAAGAAAC AGATAAACAG AGAGAAACCC |
| | TGCAAAAGGG ATTGAGCATT ATTGATCTTG AAAAAAGGAG AAGGGCTATT |
| 2030 | CACGCCGCAC AGCTGCCACC GCCGGCCCTG GCCCGGGAGA GGGGGGTCTC |
| | TGGGGATCTT AGCAGGCCAT GGTGGGGGTG TCTTGGGGCC AGCCTGAACT |
| | GAAAGACCAT CACAGCTTGC AAGCCACCTC CCTGCTCTGT TGCTGGCTCT |
| | CCTTGAAGAC AAGTGTTTTT CAATTAACCA AAGCGGTGCA TGGCAATGTG |
| | ATAGGGGAAT GAAACACAGC AACAGAATCA ATGCCCCACG CTGGGCAATA |
| | GCCGACTTTC TGTTCGCTCC CATCCCTTCC TTCCCTCCCC GCCTCCTTCC |
| | CCTGCTCCTT CCATTCCACA AACATTTATT GAGCACCCGC TGTGTGCCAG |
| | CCACTGTTCT AGGCCCTGAA GACACAGAAG TGAACAAAAA AAAAGAGTCC |
| | CTGTGCACAT CCTGGAGGGA CTTTCCCCGT GTGTGTGTGT GTGTGTGTGT |
| | GTGTGTGTCT ATACAGTGTA TGGAGACAGT GGATAATAAA AGCTGTTTAT S |
| | AGGCACTTTC TCTGAGCTGT TCCATGTGCT TCACTTATAC TCATTCAGCT |
| | AATCCTCAGG ACACCCCCGT GAAGTCAGTG ATATTAGTCA CAGAGGCCCA |
| | GAGAAGTGAA GTGACTTGCC CAAGGTCACA CAGCCAGCAA GAGGCCAAGC |
| | CCGGATTGAA CCCCAGCCGC CTGGCTCTGG AGCCTGCAGC ATAACCACAG |
| | CAGGGAACTG CCACCGGAGA CAGACATCGA CAGCCACTAG GAGATGTTAA |
| | CCAACAGGCT TGTCTTCACG GCACGGCCCC CGCTTCACCA GCTGCACTGT |
| | TTGATGAGCT TTGCAGGTCC CAGATCTTAT AAGCTCATGG TGATTGATCC |
| | AAATGATGCA GAGGTCGGCC TAAAGTTAGA AGTGGGCCCC TCTCTGCCCC |
| | AAGACAGCCC TTCACCCCAA TTCCATTCCC ACAGTTTGGG CATCCACCCA |
| | GGCTGCCAAG CCAAGCGGGG GCTGCCCGGG TTAGCAGGGA CCTGGCCATG |
| 2031 | GGCAGCAAAT TCCCCTCGTG ACTCCAATGC CCCTGCCATC TTGGGTCTGG |
| | GAGCCTTTGC ACCCTGCACG CCACAGGCTC GCCACCTCCT CCCGCCAGCC |
| | CCCAGGATGC CTTCCCCACG TGGGGGCCCT TAGTGGCCGG GACCACTTTG |
| | GTAGCAGTGC TTACCCCAGC CCCCCAAACC ATCAGTTTCT GGGGCCAGCT |
| | CCCCATGAAG CAAGAGCCCC AGGAGCAGGG ACATCTCCTT CTCTGATGTC |
| | CCTGCACCTC CAAGGTCTGG GAGACGCTCA TCCCTCTTCT CTGTGGCCTC |
| | TGCCTGGGAA AGCTTGGGGG CTTTGTAAGT CAAAGGTTGG TCTTGTGGGA |
| | ACCTCCCAGT GGGCTGTCCC TGAGCCCTGG AGCCACAGAA ACCCCTGCCC |
| | CACCCACGAT GGCTGTCATC TGAGTCTGCA TGGAGGTGTG TCTGTGCCAA |
| | GTGAGCACAC ATGCACCAAG GCGGCCCCTG GCCACCAGGG GCTCTTGAAG Y |
| | CTGCAGGCGG CCCTTCCTCT CTCTCCCACT CCCCCTGTTG GGGCCACCAT |
| | TCTGGCTGGC AAGATGGCTG TGATGGCCTC CAGCAGGCCC CCTGCCTCCA |
| | CCCTTAGCCC CTTCAAGCAT TTCCCCAGAG CCACCATCTA AACGACAGCT |
| | GTGAGAACTC CTCTGTGCCC CCATTTGTCT CCCACAGTGC CCATATTCAT |
| | CACCAGCCCA CAAGGCCCCT CCTGTCTATG GAGGCCTCCT CAGGACATCT |
| | GGGGGCTTCC TGGGCCAGGG TGGGGGGTGC CTGCTCCAGG CTGTTTCTGG |
| | CTTGTCCCCT CCCTGGGTGC TGCCTAAGCA GCGGCCTCTG TGTCCTTTCC |
| | TTGGACCCTC AGCACCCCTG CATCTAACCT ACCCCCACCC CGAGCTTGCC |
| | TCAAGACCCA GCATGGACAG GGCCGCCTAC CTGAAGGTGT GGGTGGCGGA |
| | GGGCTAAGAC ATTTGGGAGG GCTATGACAT TCCGGGACCT TTAAATTCTT |
| 2032 | ATATATTGAT CAGTTGATGA AAATGTTGTG ACCAGGGCTC TCAGGAACTT |
| | AACCATATGT TTCCCTGGAG TGATGGTTCA GAATTTGTTA ATTCAGTGTC |
| | CCAAGTGAGT TTATAGAATA ACCACCACGA ATAATGAGAA GCAACGGCAC |
| | AAACTTTGTG TACACAGGCA TTTTTTGAGA GAGAGAGAGA GCTATAATAT |
| | TTATCAAATT CTCAAGGGGG TAACTGAAAA ATGTTCAACC ATTACTTCAG |
| | AACTTCTATT ATGAGACAAA TAGTGCTGGT CCTCTTGAGA AACCAAGGGA |
| | GTGGGCTCAG CTCAGTGACT GTCTGGATCA GCCCAGAAAC AATGTAGGCT |
| | GCCATCTTGA TCTGTGCTTT TCCCCAGGGA CATGAGCAAG GGCACTTCTA |
| | GGCCAAGCAA AATCAATCGT GTTTCATAGA TTTGGATTTA GAGGGCGCTC |
| | TAAACCAGCT CAGAGGAATG GTATGTTTTA GAATCCTAGG GAAATTGAGT W |
| | CTCCAATCAT CACTTCAGTC AGCTAGGAAA GATACACTTT TGGCAGGGTG |
| | CGGTGGCTCA CGCCTGTAAT CCCAGCACTT TGGGAGGTCG ATCACAAGGT |
| | CCGGAGTTCA AGACCAGCCT GGCCAATATG GTGAAACCCT GTCTCTACTA |
| | AAAAATACAA AAATTAGCCA GGTGTAGTGG CATGTGCTTG TAGTCCCAGC |
| | TACTTGGGAG GCTGAGGCAG GAGAGTCGCT GGAACCAGGG AGGCGGAGTT |
| | GCAGTGAGCC GAGATTGTGC CACACTCCAG CCTGGGCAAC AGAGCGAGAC |
| | TCTGTCTCAA AAAAATAAAT AAATAAAAAA GATATCCTTT TAATAAAGAA |
| | AAAAAAACAA CACATAAAAC TTAAGAGTGT AATAATAGCT GCTGTTCATT |
| | GAATGCTTAC CAGGAACTAG TATGATCATA AGGAACTCTT ACAATCACCC |
| | TGAGAGGGAG GGCATGGCAA GCCCTAATTA ATGGATGAAG AAACTAAGGC |
| 2033 | GGTCATGGTG GCTCACGCCT GTAATCCCAG CACTTTGGGA GGCTGAGGAA |
| | GGAGGATCAC TTGAGGTGAG GAGCTTGAGA CCAGCCTGGC CAACATGGTG |
| | AAACCCCATC TCTACTAAAA ATACAAAAAT TAGCCGGGCG TGGTGGTGCA |
| | CACCTGTAAT CCCAGCTACT CGGGAGGGTG AGGCAGGAGA ATTGCTTGAA |
| | CCTGGGAAGT GGTGGTTGCA GTGAGCCGAG ATCTCACCAC TGCACTCCAG |
| | GCTGGGCAAC ACAGCAAGAC TCTGTCTAAA AAAAAAAAAA AAAAGAAAGA |
| | AAAAAAGAGT TATATTATTT ACGACTCTTT TAGCTTCACT TTCTTATAAG |
| | ATTGTTGTGA GAAGTGAGTA AGACACATGC AAAGTGGTAA ACTGCCTGGC |
| | TGTCATAAAA GTCTTGGCTA CTATCACGGC TGGGAGGCAA AGGAGATTCA |
| | GAGAAGGGCA GAATGACTGC ATAAGGTGAT CTGCCTGTAA CTCGTGTTTC Y |
| | TCCTTTCTAG TATAAAGAGA TAAAGGACTT CAAGGTCTTA AACTGGTGAG |
| | GGAGTATTAC AGCCTTTGTA TGGAAAGGTT TGACTTTGTG TCTCTGCTTA |
| | AACCCCCATT GTGTTGCCCC TACCAATTAT TGTGCTGCCC CCTTGTGGTT |
| | AACTGTGCTA AGACCTTTCT TTTGCTCTTC TCCCATTACA TTTCCCACCC |
| | TTCCCCACTC CCTTTTAGAC CCCTCCTCTT AGCTCCCAGT TTCCCAAACT |
| | GTGTGCCAAG GTGCCCAGAG CACCTCAGGC TTGAACTTGT GTTTTGAGTT |
| | GGTTCATGGT TTCAATATTA GATACCTACA CCCGTTTCTA CAATGTCATG |
| | TCTTTGTAAA GATGTATTTT CAGAGGTTGC TCCGATAAAA AGCAACTACT |
| | ACATGAAAAT GAATGTAGAA CAGGAAATGA CAGTGTGGTA TTGTCCAATT |
| | TGATTCTAAG GTTTGATAAG TTCTACAGTG CTCTACACTA AGTTGTAAGG |
| 2034 | TTAGCTTCTG CTTGAGCATT TCTACTCACT GGAGTTCATT ACTTTCTGAA |
| | ACTGTTTCCT GTTGAACAGC TTTAATGGTT AGAAATTTCT TTGTTATACT |
| | GATCCCAGGC ATGCATCACG GTGATTTCTA ATAATTGACA AAATTATTCC |
| | CCAGTTTTCT TAATCTCTTC TCTACCTGAC CTGGTTCATA GTAATATATT |
| | TATATTCCTC AATATGCATT CTACTATAGC AATATTTATT TTTTCATTCT |
| | CATTGGAGAA TTGTGGATAA TGTATTGACT TCAGAAAATA TACATGTGTA |
| | ATCAGTATAC AGGTAGACTA CTGTGAGGGC CACCTTAGAG GGCTGTTTTT |
| | GGAAGCTCAG GATCCAATAA ATCTAACACC CAAGACCCAT GATTAGCTCT |
| | TCATGGGTAG TTGAGGTCTT TCAGGGGGGT TTATTTTTGG TTAGTTACCT |
| | ATCTTAATCT TTTTGGTTCA TATCTCTTAT GGGGAGAGAG GTAGGGAGGG R |
| | CATGGAAAAT ATCCTATCAA ACCTCCATTT ATAATTGTGC TCACTATCTT |
| | ATAAATAAGA CAAGTAAATT TTTCTAAAAC TTCGGAAGTT TTCTTGATAA |
| | TTATATAGTC ATTTAAGGAT TAATGAATTC TCTAAAGTTT AGCCAGTTAT |
| | CCATATTAAT GGTAGCTAAA AATTGAGAAT GCCACAAAAA TTTCAGACAT |
| | CTGACTTTTT AAAAATTAGA ATTTTTTGGC AGGGTGTGGT GGCCTGTAAT |
| | CCCAGCACCT TGGGAGGCCG AGGCAGGCAG ATCACTTGAA GTCAGGAGTT |
| | CCAGACCAGC CTGGCCAACA TGGTGAAACT TTGCTCTACT AAAAAATACA |
| | AAAATTAGCT GGGCCTGATG GTGCACACTT ATAATCCCAG CTACTTGGGA |
| | GGCTGAGGCA GGAGAATTGC TTCAATTTGG GAGGCAGAGG TTGCAGTGAG |
| | CCAAGATGGT GCTACTGCAC TCCAGCCTGG GTGACTTGTG ACTGGGCAAC |
| 2035 | CCTCTAGAAT CCTTATTGCT GTTCCCACAT TCTGATCATA AATTTGGATA |
| | AATTCACTGC TACTCAGGCT TCAAGCTGAT ATAATGAATA GAGGGAAAAT |
| | AGGCACCCAA AGTCAATAAG AAGCACAGGT GATGATGCCC CTGTGATTTG |
| | TGGGATTGTA AAAATACATT GTCTTCTGTT TTCCCTTTTT GCCCAAATCC |
| | AAATTTAGAA GAGAACATTC TGGGAATGTA TACGTAAGAA CTGCAACATG |
| | AAGGAACAAA CAATAACCAT GATAAAGCAA GCAGGCAAAC AAAAAACAGC |
| | CCTGTGACTA TAACCACAAA GCTGGGCAGC ATATAATTCT AATTTAATAG |
| | TAGCTGGAAA AGGCTTCTCT CTACTCTATA GTCATGGCCC CCATGCTCAA |
| | CATGGAACTC CTATATTTTA GCTTTTAAGT CATCATAAAA TATTAAGGGG |
| | CCCATATTCT TTGACTCCCT AGGTTTCCAG AATTACTTGG ATTTTCTGAG S |
| | ACATTTTCAT TTCTTAGTGA CACATGCAAT GTCAATAGAT TTTGGAAAAG |
| | TATTTCTCAT GTTCCCTATT TACCTACAAA ATTCTTCTAT GCATCTTTTC |
| | TCCAAGAAAG ATTTAAGGAT GAAGAAATTA CTATGATACA TTAAGAAAGG |
| | CCAAAGGAAG TATACTCTAA TATATACCAC ATTATGTACT TAGGCTAAAA |
| | AACGTTTCAT TCCTTTGTGA TATTTTCTTC CTTCTGTGTA AGCAATTTGT |
| | CATATTTTCT AAAACCATGT TTTTAAGATT TTTCTTGGCT ACTCTTTAAA |
| | CTTATTTACT TCTGAAGTAT GAAAAACACA GTTCTCTTAG TTTGAGTGCA |
| | ATGTGCACAT ACATTAGGGA AAGAACAGCC TATTCAATAA ATAATTCTGG |
| | GAAAACTGAA TATCAACAAG CAGAAAAATG AAGCCAGACC CCTCTCACCA |
| | TATGCAAAAA TCAACTCAAA ATGAATTAAA GACTTAAAGC AAGACCCAAA |
| 2036 | TTCTTTCTTATTTATTTGTACATTATCTGTTTGTATCCTTGAATTTTCTCTGGGATGTTAT |
| | AAAAGAAAAATATTACAAGCATAATTTCTGACACAATGTTTTATAACTGTTTTCAAAAG |
| | CAATCTATTTCTCTCTTAATTTCTATAGAACCCATTTTAGTCATTCCTATGATGAATGAA |
| | AAAAAACTCACAATTAGTTGTCAGTTTCAGAAAGGAGTCTCAGAAGCATCTTAGAACC |
| | ACCTAGGCGGTCTTATAGGCGTCACAGAAAAATATTCCACCTTACTTATTCTTATCTTCT |
| | TCAAACTGACTCCAAATATAATAAGCAGGCTTAAGTTATATAAGGTTCCTTAAAAGTTG |
| | TTCTCAAAAACAATTAAATGCAGACTGTCCTCAGTAAAACTTGCATTGGCCCAGTCCAT |
| | TTTACCAAGTTTGCTCCTTCTTACATGTACGACTGCCCCTGTGACAAGTTTAAGCCTACG |
| | TTAATTAATCATCTTTCCATAACANTGAGACATATTCATCCAACCCTGAGAGGGATAAG |
| | GGGTAACAGGCAAGATATATTCAAATTGTGACATTTAAGGTCAACTTGACTTTAAAACT |
| | CATATACTGCTCAGTTTTCATATACGTGGCTGAAAACAAGTCATCACAGAGAACTGGTC |
| | ACACTGTTAGAGGACCAAAATAGTAAAATATGAACAATTCTTCCATATTTAATGATATT |
| | AAATATAACAGGTCCTATGTCTATGCAATTGAAGTCAACATGTCACTAATTAGGGCTTC |
| | CTTTGTTATCAAACTTGCTGAGAAGATAGAACAGTGAGGAAAAAATAGCAGGAATTGA |
| | GCTGTACAGAATTAAAGACATTCTTATGAACCTTAAGACTTAAGGTCAGTCACACTCAT |
| | ATGACATTTGGACTATTAGACAACTCTTCTGACCTCAAATTTTGCTCTTACCAGTTGGTG |
| | ATAATCCTTTGGTAAGAGAGCATGTTAAAAGTAACCAAATCAGATAAGGACTA |
| 2037 | TTCTGAGCAC TCACATTTTA ATGCTGGGAA TTTTATGCTG AGGTTCAGTG |
| | CTTTTCAGGG AGCCATGAAC CCCCTGCAAT GACTTGCAGA CTGTAGGTAT |
| | TACATGTAAA ACTTGTGTGA GTAATTTCCT GAAGAAGGGG TTTCATTCTT |
| | CAGATCCTCA AAAAGATCTA TGATCCGGCC AGGCGCGGTG GCTCATGCCT |
| | GTAATCCCAG CACTTTGGGA GACCAAGGCG GGCATATCAG GAGGTCAGGA |
| | GATCAATACC ATCCTCGCTA ACACAGTGAA ATCCCATCTC TACTAAAAAT |
| | ACAAAAAATT AGCTGGGTGT GGTGGCGCAT GCCTGTAATC CCAGCTACTC |
| | GAGAGGCCGA GGCAGGACAA CAGCTTGAAC CCAGGAGGCG GAGGTTGCAG |
| | TGAGCCAACA GAGTGAAACT CTGTCTCAAA AAAAAAAAAA ATATATATAT |
| | ATATATATGA TCCTAAAAAG ACATTGATAT TAAAGAGTAA TGATGGGGAA R |
| | GACCACCAAG TGATCATTTA TAAGATGCTG CGAGAATTGC TAAAGTTGAG |
| | GGATGGATTC TGCATTCCAG GAGAACAAAG GAGGTAGATC CCGATTAACT |
| | GAGTAGCAGG ATATTGAGGG AAGCTTCTTG TAATGATTCA GCTGAGTCTT |
| | ACACAATGAA TGAGACTTGG ATAGATGAAC ATGCATTAGG AGACATCAGT |
| | TCTGGTCAGA GGAAACAGCA AATGCAAAGG CCCAGAGCCA TAAGAGATTC |
| | AGATGCACTA AAGAAATATC CCATCATTTA GGATGGTCGG CATATACACA |
| | GAGGGCCTAT AAGGAAGGAG AAGAAACTGA GGTTGTAGAA GCTGGTAGGG |
| | ACTGCATGCT GAAAGGCATT GTAAATCTTA GACTAAAATC TGTGGGAGAC |
| | TTTGAACAAC CTAAAGATTT TAAGCAGAGT GATGCAATGC ATTTGTCTTT |
| | TGAATGCAAA TATTGCTGGG GCGGGTCTTT TGTGTTATCA GCTAATGATT |
| 2038 | ATATTCTTTC CTGTCTGCTG CCTTGTAAGA CGTGACTTTC GCTTTCTGCC |
| | ATGATTGTGA GGCCTCCGTA GCCATGTGGA ACTGTGAGTC CATTAAACTT |
| | CTTTTTGTTT ATAAATTACT CAGTCTCATG TATGTCTTTA TCAGCAGCAT |
| | AAAAACAGAC TAACATAACC CCCCTTTCTT CCTACCCCTG TGATTTGGGT |
| | GGATATTAAA ATTTTGCAAG CCGCCACTCT AGAGAAGTAC CACTCAAACC |
| | ATGGTCTGCA GACTACCAGC ATTAGCAAGG CCTGGGAGTT TGTGAGAGAT |
| | GCAGATTCTT GGTGCCCATC CAAACCTACA GAAGCAGAAT CTCTGGTGGC |
| | AGGAACCAGC AATCTGTGCT TTCAACAAGC TCTCTGAGTG CTTCTTCTGA |
| | ATGTTAAAGT ATAAGAACCT CTGCTGCACA GGGAGACCTC ACTTTCATTA |
| | TTCTCATTTC ACAGATGAGA AAAATGAGTC AGTAGGAGGA AAAGTGATTG N |
| | CTCAAGTGCT TAATAAGTGT CTAAGCTAGG ATTTGACCCT AGGGCTTGTG |
| | AATCTGGAGC CCATAGACTC AACTCCTGTG CTATACTGCC TCTGTGTCTT |
| | TGGTCTGTGT TCTCTTCTTT TTTTTTTTTT CTTACCACCC TCCAAAATTC TTATTTATTT |
| | ATTTTCTATC ACCGTACTTA CTGGTGGAGT GGAAGTCCCT TGGAAGTGGG |
| | GGACGTGTCT GATTTGTGCA TGGCTATTTT GCGATTTGCC ATTGCTGGAA |
| | TATTTAGCAA GCATCAATGA GTCCTGGAGG GCTTTTAAAC CATAGATGAT |
| | CAGGGCCCCT GCTTGGTCTA GGATGGAGCC TGCAATTTTG CATTTGTGAC |
| | AAGCTCCCAC GGATGCTGGT GCTCACTTGG CATAGCAAGG GGTTATGCTA |
| | TTTTTCAAAG GCAAGAGAAG TGTGCATGTA CTTGAGAGGA GAAGGGGTCG |
| | GTCACCATTT ACTTTGTGCC TCTTGCTACC CTTCGTTGCC |
| 2039 | AAAGCTCAGT CCCAGATCCT CGGCCATGCT CCTGCTGCTC CTGGAGGCAC |
| | CTCTGACTCC TGGGCAGAGA GAGCCCCCAT TAAATAGAAG ACCAGCTCTC |
| | ACAGTGGGTG ACTTAATCAC TCAGTCTCCT CCTTCCCTTT TCCTCTCCCC |
| | TCCCCTCTTC AAGAGCACAC TGTGGCCCTA CCCTGCTAGA GAATGCACCG |
| | CCTACAACAG AAACCAAGTT TGGTTTGAGG AAAAAAAAAA AAAAAAAAAA |
| | AAGAAGAAGA AGGAGAAGAA GAAAGCTTTC CCAAGCATAT TTATATACAG |
| | TATGCTCATG TGCTCCTTCC TTCGTTTACA GAAGGAAGTT AGGAAAGTCC |
| | CTGAAGGAGG AGAGAAAGAA TTCATCAAGT CAGTGGGTGG GGCAAATTAA |
| | AATATACCTG TTCCCTGCAC TGGAGGCTTA CCAGCTGTGC CAGTCTGGGG |
| | AGTGTGCTTC TGGAAGTGAA AGTGAGGGAT GAGAGGTGTG TGGTTTGCAG R |
| | TTGGGAAACG GAAATCACAT TTGCATCAGC TCTTTGCAAA GTGCTGCCTA |
| | GCCCTCTGTC ATTTTGAACC TCATAGAAAT TCATTCTCAG TGTACAGATG |
| | GGAATAGCAA AGTTCTAAAA GGTGAAGGCA CTTGTCCTAG GTCATCCAAG |
| | GATGAAGACA GAGGAGCTAG GAAGATGACC TAGTTCTAAA TCACGGCTTG |
| | GAGTTGTAAC CTCTAGCACA TGACTGCCCA TGAAAGGAAA GTATTTCCAG |
| | TCTGCATTGA CCATTGTTTA ATCAGAGTAT GAGGCCACAG ATCGAGGTGA |
| | CTGTCTGTGA GGGTAGAACA TTAACCACTA CTCCCTGATT AGTCTAAAGT |
| | TAATTGATCA TGTGATGTGC TTTGCCTGCA GTTGGGTGTG GGGGCCACAA |
| | CATGTAATAA AAGATTATAT TTATTAAGTG CTTACTTTGT GCCAATCACT |
| | GCTCTAAGTT AAATACATCA ATAAAATTAT TCAATCCTGA GATAAATTTT |
| 2040 | CTCAGCA CTTTGGGAGG CTTAGGCAGG AGCATCGCTT GTGTCCAGGA GCTTGAATCT |
| | AGCCTGGGCA ACACAGTGAG GCCCCATCGC TACAAATAAT CAAAATTAGC |
| | CAGGCGAGGT GCATGTGCCT GTAGTCCCAG ATACTCAGGA GGCTGAGGCA |
| | GGAGAATTGC TGTCCTCCTG CACCAGAAGC CTCCAGTTGC TTTGTGTCTC |
| | AGTGGCTCAC CCTATTCTGG ACACTTTGCA TAAACAGAGT CCCACAGGCT |
| | TGTCCTCAGG TGTCTGGCTC TGTCACTCGC ATAATGGCCT TGAGGTTCTC |
| | CACGCCGCAG CGTGCGTCAG GACAGCGGCC TCCTTCCTTT TTCAGGCTAA |
| | TTCTCCCCTG CACGCATGGA TCACGTTTGG TGCATCCGTC CACCCACGAG |
| | GAACACTCGG GTGGTTCCTA CCTTCTGGCT GCCATGAACA TTCACGGACA |
| | GGCATTTGTT TGAGTCCCTG TTCTGAATCC TCTGGCCTAT ATC S |
| | CTAGGAGAGA ACTGCTGGGT CCTGCAGTGC TTCCACGCTG AGCTTTTCGA |
| | GGAACTGCCA CAACGTTCCC CATGGCAGCT GCGCCCTTCT GCTTTTCCGC |
| | CACGATGCAC AAAGCTAACA ATGCCTCTGG TCTCTGTGTG CAGTGCCCTT |
| | CAGAGAGCCG TCCTCACCGG CAAACAAGCA GCGGCATCCC CCTGGGAGCT |
| | TGTTCTGAAT GCCCGGCGCG TCTCCAAACA CACTTGGAGA AACCACCTGG |
| | ACGGTGCTCA ACCTTGAACC TCTGAAGCTT TTAGACACTG TCCCGATGCC |
| | AGGTGCCAGC CCAGAGCTTC AGGTGCAATG GGTCTGGGCA TGGGGGCTTT |
| | CATAGGCTGC CCAGATGACC CCACTGTGTG GCCGGGGTTG AGACGACAGC |
| | CCTGGGAGAA GCTTCTGCTG CCACCTGGGC TGCTCTGCGG GGAGTATGCT GGC |
| 2041 | AGACACACTT CGTGACCTCA CTAGGATTTG CTTTACAAGA TAAGATTTTC |
| | CTGGCAAGCA TTGAAGTTAG TGCCCAGATT CAAAACAGCA AACTTAAAAC |
| | TATTATCTTC ATGCTTGCTA TAATCAGGTG TACCGCCCAA CTTCCCATCC |
| | CTACCTCCCC TTCCCTAGTA ATCCAGAGGT ACTCAAGGTC ACTACTGGCT |
| | ACTTAAGGTT TACATCCACT CCCAAGGAAA CACCAGCAAC CAAAAGAATA |
| | CTTCCCAGAT GGAAAACCCG TCATTTGACC TTGGAATGCT CTGCCTCCCA |
| | TAATGACCAA AATAAGGTCC GGCCAGAGGG GCTTACGTTT TTTGGTAAGG |
| | AACAACACAT TGTTTTAGAG AGATAATCTG ACATAACTCT GCTAAGCTCT |
| | GAGTGTCTAT GCATGGTACC ACAGCAAACA CCCTATGTCC CAAATGGAAA |
| | CTAGCAGGGT GAAAAAATTG TTCATCTAAG ATGACATTAA TCTCTGTATG H |
| | CTCCTTGGTA CAGTTCTTTT CCACAACATG CAGCTTAAGA TTCAACAATT |
| | TTTTCATTGT GTTTTGTGTT TCCTGAAGCA TTCCTCCAGC TACCTGAAGC |
| | TCTCACTGAT TAAAATAGAC CTCCTTTACT AAAAGGGGTA AACTGAGGCA |
| | CAGAAGAACA AAGCAATGTG CCTTATAGTT CAGAATAGGT AAACAAAAAT |
| | TGGACATCTA TTATTTTCAA ATCTCTTTAG ATTAAAAGAA AAAAAAATGT |
| | ATTTCTCAAA GTCCAAAGTC AACCTTCTCC AAAGGGAAAC TGCATGTCTG |
| | TCTAGTAACC TATAGGATCA TTCACACTTT TTTTTCCTTG AGAAGGAACT |
| | AACTTGCCAG CCTACCAACA ATTTTGCCAG TGCCTTGGCA TCAGGCATAT |
| | CTGCACAACC TTTCCAGAGC CTATATCATA GTGATCTGTT CAAGCTGCTC |
| | AAAGCTCAGT AATCATACTA ATCTGTGCAT AGTGACCAGT GCTACCTGCT |
| 2042 | GCTCCCCCAAAGATATTTACATCCTACCCTGTGAATATGCTACCTTACATGGAAAAAGG |
| | GGCTTTAAAAATATGATTGAGTTGAGAATCTTGAGATGGAAAGTTATCCAGTATTATCT |
| | GAGTGGGCCCAATGTCCTCACAAGGGTCCTTATAAAGTGAGAAATAAGAGGATCAAAG |
| | TAAGTAGTAGGTGATGTGACAGCAGAAGCAAGAGGTTGGAGTGATTTAAGGAAGTGGG |
| | AGATCAGGAAGTGTTAAGCACAAAGTTAACTTATACCTTTAAAAAAAAAGGAAAATTA |
| | AAGACAACAAGCAGATATATACATTATATAAATAAACACAAGAACTTGTTAAACTTGT |
| | GAAACACAGGACTACCCAAGACAAATGTTTCCCTTTCCATTTCTACTCTAGGCTATATG |
| | AGGAATCTATT |
| | TTTCTCTTTTTCTTAGAACTCTTACTCTGCAGGATCAAAATGTCAAGCAGTGACCACATA |
| | AGTGGGAAGTAGATCAAGACNTGCTTATAATTACATTCTTGTGGTTGGTATAAAATTCT |
| | GTACCACAGCCATTCCTTAGAAGCCAGCTCATGGGATCAAACAGACAAGCAAGCTCAC |
| | TTCTCAAGCCCACGATGTTACGTATTAGTCCATATTGGTGAGTTCCTTTGTGCCCAAACT |
| | TAGCTGCCGCACACTACAGTGATAGGAAACTGCCAACAGTTTTTTGTCAAACCCACTAC |
| | AGTGGATCCCACAATGGGCTGAGACCTCGTCTAGAGTCATCAGGTTTTCCAGGATGTGC |
| | AGATGGCCTGGAAACTTGGCCAAGAGGTAGGGATCAGCACTCTTTAGTAAGCTGTATA |
| | TCCACCAAATGCTGTGACACAAGGAATGGGCCTCAAAATGTTTCAGGCCAGCACAGCT |
| | TTGTCTTTGCAAGTCATTTTTCAGTGGCAGTTAAATATTGTGGCAAAGCCAAGGTACAG |
| | ACTCACGTGCATTAAAATATGCTTATGAGTAATAATACCATAAGTATTTCC |
| 2043 | GGGGTCAGGTTGCTGGTCCCAGCCTTGCACCCTGAGCTAGGACACCAGTTCCCCTGACC |
| | CTGTTCTTCCCTCCTGGCTGCAGGCACCCCCAGCCAGAACACGCAGTGCCAGCCGTGCC |
| | CCCCAGGCACCTTCTCAGCCAGCAGCTCCAGCTCAGAGCAGTGCCAGCCCCACCGCAA |
| | CTGCACGGCCCTGGGCCTGGCCCTCAATGTGCCA |
| | GGCTCTTCCTCCCATGACACCCTGTGCACCAGCTGCACTGGCTTCCCCCTCAGCACCAG |
| | GGTACCAGGTGAGCCAGAGGCCTGAGGGGGCAGCACACTGCAGGCCAGGCCCACTTGT |
| | GCCCTCACTCCTGCCCCTGCACGTGCATCTAGCCTGAGGCATGCCAGCTGGCTCTGGGA |
| | AGGGGCCACAGTGGATTTGAGGGGTCAGGGGTCC |
| | CTCCACTAGATCCCCACCAAGTCTGCCCTCTCAGGGGTGGCTGAGAATTTGGATCTGAG |
| | CCAGGGCACAGCCTCCCCTGGNGAGCTCTGGGAAAGTGGGCAGCAATCTCCTAACTGC |
| | CCGAGGGGAAGGTGGCTGGCTCCTCTGACACGGGGAAACCGAGGCCTGATGGTAACTC |
| | TCCTAACTGCCTGAGAGGAAGGTGGCTGCCTCCTCTGACATGGGGAAACCGAGGCCCA |
| | ATGTTAACCACTGTTGAGAAGTCACAGGGGGAAGTGACCCCCTTAACATCAAGTCAGG |
| | TCCGGTCCATCTGCAGGTCCCAACTCGCCCCTTCCGATGGCCCAGGAGCCCCAAGCCCT |
| | TGCCTGGGCCCCCTTGCCTCTTGCAGCCAAGGTCCGAGTGGCCGCTCCTGCCCCCTAGG |
| | CCTTTGCTCCAGCTCTCTGACCGAAGGCTCCTGCCCCTTCTCCAGTCCCCATCGTTGCAC |
| | TGCCCTCTCCAGCACGGCTCACTGCACAGGGATTTCTCTCTCCTGCAAACCCCCCGAGT |
| | GGGGCCCAGAAAGCAGGGTACCTGGCAGCCCCCGCCAGTGTGTGTGGGTGAAA |
| 2044 | CACAAACCAT ATAAGACATT ATTGTTCCCA TTTTACAGGT GGAAATCTGA |
| | AGCTCTGAGA GGTGGAATGA GTTGCCTGAA TTTACCAGAA TGTGGGATTT |
| | ACCAGAATGT TTACCAGAAC GAATTTACCA GTCAGGCTTT GAGGCCAGAT |
| | CTGTCTGATC CAAAATCAGT GCTCTTTCTG AAATGCTGAT GCAAAAGGAA |
| | GTCATCAGAA CTCTATAGAC CAACCCTGCA CATTTGTTAC CTTCTCCAGA |
| | AGTGCTTTCG TTGCTGATAT ACCAGACGTC TTTTGTTAAA ATTAATCTAC |
| | TACCATCACC GTATGGGGAA TAACTCTGGC TAGCCTAAGA AAAGCAACCT |
| | TACATAGGTA AAAATAATTG CACCCCTTCC ATCTGCCAAA AGAGTTCTAT |
| | TGTCGGTTTC CCAGGCAATA TATGTGCTTT GGGAAATATT GCCATTAGAC |
| | AATCTGAGTG TGGTTGCAGT AAAGTATTCT AGAATGACTG GAATACCCAG M |
| | AAACTGGGTA AGAATCATGT GTTCTGCTGT CAGCAGACCT TGTCTGGGCC |
| | TGGCAAACTC TGCCTCTGGG GTTAGCAAAA CTCCCTTACG CTCCCCTCTT |
| | TCAAACAAGA GTGATTATCA GTTGTCAAAG TTGTTACTTC TGCAGAGAAG |
| | GAAAAGTGGT TCTGGACACT GTCCACAGAT TGTTTCCTCC TCTGGGGCAA |
| | GAGGATTGGG AGAGGGGTAT GCACATGCTT TTCTCAATTT GTGGTTTTTG |
| | CTTGGGTTTG TGGTTTAAAT CCCTAATTTT GGCAATCATC TCTGCCACAG |
| | ACCATCCAAT CCATGCAGGT GGGTTCATTC CATACTCTGT GAAATATATT |
| | TTGATGTTAG AGGGGTGGGA ATGTCTGATT TATTGTTTCA TCCTCGTCTC |
| | TGGGGAGGGG AAGCAAAGCT AGTAGTAGTT TTTTTTTCAA GGGCAATCTC |
| | AGGCCATTCT AGAATGCTTC ATTGCAGCCA CACTCTGATT GCCTAATGGC |
| 2045 | GTGGGAGGAT CACTTGAGGC CAGGAGCTTA AGGCTAGCCT GGGCAACATA |
| | GCGAGACCCC ATCTCTACAA AAAATAAAAA ATTAGCTGGG CATGGTGGTA |
| | TGTGCCTGTG GTCCTAGCTA CTTGGGAGGC TGAGGTAGGG GCATTGAGCC |
| | TAGGAGTTCA AGGCTGCAGT GAGCTATAAT CACACCACTG CACTCCAGCC |
| | TGGGTAACAG AGTGAGACCC TGTCTCAAAA AAAAGAAAGA AAAAGAAAAA |
| | GAAAAGGGGT ATTTATTGAA CACCTACTAT GTTTCAGGCA CTGTGCTAGA |
| | TCCTAAGTGA ATATCAGCAC ATGAACAAGA CAAAGGCGAA AAGTTACCAA |
| | ACAAGTCTAA GTTGATTTCG GTATATGCAT CTTCCTGACT TCTGGTCCCG |
| | TGCTTTGACC ACAACCCTTC ACCACTAGAC CAGACTTCCC CAAATAAACA |
| | ACTACTTCTG CATGCTGGGG ATGGGCTGTG TGCGGCAGCA TTTACGTAGG Y |
| | GGTACAGACA GCAGCCTTTC ACTTAATGTT GCAATAACAC CAGGCTAAAC |
| | AATGTGCACT GACTTCAAAA GTGTGGGGTC AGGTCTCCTT CAAGTGCCAC |
| | AGGGAGAGTG CAAAGTAGGA AAAGTCTATC GGATGAGGAA ACACTGTAGA |
| | GGGAAAGTGA ATTTTTTTTC AATTTGGGTT AAAATTCAGA TGTGGAATTC |
| | TACCCTCCTT TTCACTTTTG GATCCCCAGA TAGGAGGAAC TCAGCACATA |
| | GATAATCATG AACTACACAC ATTTTGGTTT TATATGCTCA GACTTGTCCA |
| | GAGCATGAAA TCCCTGCCCT GTTGGAAGGC AGCGCCGTGC TCACGGAGGC |
| | ACACAAGCAC CTGTCTCAAA GTCACCCTGA CCTGCAGATC TGCAAATGGC |
| | AAAAATAATT TCACATGTTT GTTCTGATTT GTCTTCATTT TTAGGCTACC |
| | TTGTGTGAGC TCCACTTTTT AGAATGTGAT TTTGCAGTCC TGAAATGGAT |
| 2046 | GACAGGAGCT GGGCAGCAGC AGGAAGACGG GGGGCACCCC TCCTGGCGTC |
| | CTGGCCGCCG GGACACCCCT CTCCGGGAGA GGACACTCGG TGCCCCTCCG |
| | TGAAGCCGGT AACCTCGGCT GCGCCACCTC CCCCACCCGC GCGCCCGCGC |
| | CGGGAAAGCC AGCGTGGCCC CAGTCTCCGA ATTTCTCGGA ATGAACAACA |
| | GCAAATTGAA TCCAGGGCTC CGGCGGGGGC CGCCTTTGCT GGCCACTGGT |
| | TCTGCAGCCT GGCGAGACTG CGCTTAGTCC CCGCCTGGAG TGCCTCCCGA |
| | AGGCCTGGCT GGAAGCTAGA CTCAGTCCCT TTGTAGTAGC AGCGGTGGAG |
| | CAGGGACCAG CGTCTTGGAC GGGTGACCCA GACTCAATTC CGACTTTGGC |
| | CGAGGGCATG TTTGACACTG GGTGTGAAAT CTAGACAGTT CCCGCGGTCA |
| | TAGAGATGGG GCAGCAGCAT TCAGGCAGGG CTCAGAACTG TCCGGGTCCC R |
| | TCAGTGTTGG GGCGGAAGAG GAAGAGTGTC AACTGAGCCA GCATTTATT |
| | TATTTTATTG TTGTTTGAGA CAGGGTGTCG CTCTGTCGCC CAGGCTGGAG |
| | TGCAGTGGGC AGCATCTCAG CTACTGCAGC CTCCGCCTCC CGTGCTCAAG |
| | CGATCCTCCC ACGTCAGCCT CCCAAGAAGG TGGTATTATT GGCCACGCCT |
| | GGCCAATTTT TGTATTTTTT GTAGAGAGGA GGTCTCACTG TGTTGCCCAG |
| | GCTGTTCTTG TACTCCTGAG CTCCAGCAAT CCACCCGCCT TGGCCTCTGA |
| | AAGCGCTGGG ATTACAGGCT TGAGGCACCG TGCCCAGCTG AGCCAGGCTT |
| | TTAAAACCGG TTCTGTCCCT ACCCAGAGAG CTTCTTACCT GGCGGCCTCC |
| | TTAACCTCTG ACACCAGCTG ACCACGCTTA TGAGCCAAAC ATAAAAACCA |
| | AAAGAAACCC AGGCTTGGTG GCTCACGCCT GTAATCCCAG CACTTTGGGA |
| 2047 | tatccac cagccttggc ctcccaaagt gctaggatta caggcatgag ccaccacgca |
| | tggcctgtct tttcttcttg gtcattttcg ctaaaggttt gtcaattttg ttgatctttt |
| | ttgttgctga tctctattgt tttcccattc tgtttcattt atttccattt taacctttgt |
| | ttcctttttt ctgctggttt gggtttaatt tgctcttttt ttcccctaat tttTCAAGGT |
| | ATACAGTTAA GTTATTGATT TGAGATCTCT TTTTTCTTtt cttttttttt tttttttttt |
| | ttttttggtt gctgttgaga tggagtctcc ctctgtcacc cagactggag tgcagtggca |
| | tgatctcagc tcactgcagc ctccgccgcc caggcgattc tcctgcctca gcctcctgag |
| | tagACGTTTC CCGGCCAagg tgtttctttt tgaatgtaag catttacagc tacagatttc |
| | cctctaaaca ctgctttcac tgc D ttccataaga ttgttttttg ttgttgtttt |
| | gttgtttgag acacagtctc actctgttgc cgtttggaga gcagegatgc gatcatagct |
| | ctgtagcctt gagctcctgg actcaatcag tcctcctgcc tcagcctccc aagtagctgg |
| | gactacaggt gtacaccact gcacctaact aatttctttt ataagttttt gcagaggcca |
| | ggcacagtgg ctcacacctg taatcccagc actttgggag gccaaggtgg gtggatcacc |
| | taaggtcagg agttcgagac cagcctggcc gacagggaga aaccccatct ctactaaaaa |
| | tacaaaaatt agctgggcgt ggtggcaggt gcctgtaatc ccagctactc aggaggctga |
| | ggcaggagaa tcgcttgaac ctgggaggca gaggttgcag tgagccagga tcacaccatt |
| | gcactccagc ctgggtaaca aaagcaaaac tccatctcaa gaaaagaaaa |
| 2048 | CATCTGGCTACCTCGGCCTCCCAAAGTGCTGGGATTACAGGCGTGAGCCACCAAGCCCA |
| | GCCTAACGTTACATTTCTTTCTTTCTTTTTCTTTCCTTTTTTTTTTTTTTGAGACAGACTCT |
| | CACTCTGCTGCCCAGGCTGGAGTGCAGTGGCATGATCTTGTCTCACTACAACCTCTGTC |
| | TCCCAGGTTCAAGCAATTCTCGTGCCTCAGCCTCCCCAGTAGCTAGGATTACAGGCACC |
| | CACCACCACCCCCAGCTAACTTTTTATATTATTAGTAGAGACAGGGTTTCACCATGTTG |
| | GCTGGGCTGGTCTTGAACTCCTGACCTGAAGTGATCCACCCTCCTCAGGCTTCCAAAGT |
| | GCTGGGATTACAGGCATGAGCCATGGTGCCCGGCTATATTTCTAAGTTTATTTTGCTTCA |
| | TATACCAGGAACCGTAGTTTGTTTCACATGTGGTCTTGAAAATAGTTTGTTCTGTTCTGA |
| | TTATAAGTAACATGTGTTTATTGNTTTTTTTTAAAAAAGGGTAAATATCCAGGTGCAGT |
| | GGCATGTGCTTGTAGTCCCAGCTACTCAGGAGGCTGAGGCCGGAGGAAGCTTTTGAGC |
| | CCAGGCATTTGAGACTGTAGTGAGCTGTGATTGTTCCACTGCATTCTAACCTGGGTGAT |
| | AGAGCAAGACCCTGTCTCAAAAATAAAATACAATTAAATTAAATTTAAAAATTAAAAA |
| | ATTGATAAATATAGAAAAAATGTGAAAACCAAAGGGGAACGCCTCCCACTACCAGAGA |
| | TCATTTCTATTTTCATTCTGGAGAATTTCCTTCTAGTTTTTTTTTAAAACGCATAAATATG |
| | GGAAATACACAATTATGGGAAATATACAACTTCCCATAATGAGTTAACACACCATTTAC |
| | GATTTTGTGTCCTGCTAGTCTGTCATTATGTCATTAGCGTTTATCCCTTGCATAAAATGC |
| | TTCTCAGATTACTAAATGGCAACATACTATAATATTGAACTTTGTGGATATC |
| 2050 | MAEDLGLSFGETASVEMLPEHGSCRPKARSSSARWALTCCLVLLPFLAGLTTYLLVSQLRA |
| >sp\|O95150\|TNF15_ | QGEACVQFQALKGQEFAPSHQQVYAPLRADGDKPRAHLTVVRQTPTQHFKNQFPALHWE |
| HUMAN Tumor | HELGLAFTKNRMNYTNKFLLIPESGDYFIYSQVTFRGMTSECSEIRQAGRPNKPDSITVVITK |
| necrosis factor | VTDSYPEPTQLLMGTKSVCEVGSNWFQPIYLGAMFSLQEGDKLMVNVSDISLVDYTKEDK |
| ligand superfamily | TFFGAFLL |
| member 15 | |
| OS = Homo sapiens | |
| OX = 9606 | |
| GN = TNFSF15 | |
| PE = 1 SV = 2 | |
| 2051 | MQLTKGRLHFSHPLSHTKHISPFVTDAPLRADGDKPRAHLTVVRQTPTQHFKNQFPALHWE |
| >sp\|O95150-2\| | HELGLAFTKNRMNYTNKFLLIPESGDYFIYSQVTFRGMTSECSEIRQAGRPNKPDSITVVITK |
| TNF15_HUMAN | VTDSYPEPTQLLMGTKSVCEVGSNWFQPIYLGAMFSLQEGDKLMVNVSDISLVDYTKEDK |
| Isoform 2 of | TFFGAFLL |
| Tumor necrosis | |
| factor ligand | |
| superfamily | |
| member 15 | |
| OS = Homo sapiens | |
| OX = 9606 | |
| GN = TNFSF15 | |
| 2052 | MRRFLSKVYSFPMRKLILFLVFPVVRQTPTQHFKNQFPALHWEHELGLAFTKNRMNYTNKF |
| >sp\|O95150-3\| | LLIPESGDYFIYSQVTFRGMTSECSEIRQAGRPNKPDSITVVITKVTDSYPEPTQLLMGTKSVC |
| TNF15_HUMAN | EVGSNWFQPIYLGAMFSLQEGDKLMVNVSDISLVDYTKEDKTFFGAFLL |
| Isoform 3 of | |
| Tumor necrosis | |
| factor ligand | |
| superfamily | |
| member 15 | |
| OS = Homo sapiens | |
| OX = 9606 | |
| GN = TNFSF15 | |
| 2053 | MFCSTSAVNSCARCFFHSVCPAGMIVKFPGTAQKNTVCEPASPGVSPACASPENCKEPSSGT |
| tumor necrosis | IPQAKPTPVSPATSSASTMPVRGGTRLAQEAASKLTRAPDSPSSVGRPSSDPGLSPTQPCPEG |
| factor receptor | SGDCRKQCEPDYYLDEAGRCTACVSCSRDDLVEKTPCAWNSSRTCECRPGMICATSATNSC |
| superfamily | ARCVPYPICAAETVTKPQDMAEKDTTFEAPPLGTQPDCNPTPENGEAPASTSPTQSLLVDSQ |
| member 8 isoform | ASKTLPIPTSAPVALSSTGKPVLDAGPVLFWVILVLVVVVGSSAFLLCHRRACRKRIRQKLH |
| 3 | LCYPVQTSQPKLELVDSRPRRSSTLRSGASVTEPVAEERGLMSQPLMETCHSVGAAYLESLP |
| (NP_001268359.2) | LQDASPAGGPSSPRDLPEPRVSTEHTNNKIEKIYIMKADTVIVGTVKAELPEGRGLAGPAEPE |
| | LEEELEADHTPHYPEQETEPPLGSCSDVMLSVEEEGKEDPLPTAASGK |
| 2054 | MRVLLAALGLLFLGALRAFPQDRPFEDTCHGNPSHYYDKAVRRCCYRCPMGLFPTQQCPQ |
| tumor necrosis | RPTDCRKQCEPDYYLDEADRCTACVTCSRDDLVEKTPCAWNSSRVCECRPGMFCSTSAVN |
| factor receptor | SCARCFFHSVCPAGMIVKFPGTAQKNTVCEPASPGVSPACASPENCKEPSSGTIPQAKPTPVS |
| superfamily | PATSSASTMPVRGGTRLAQEAASKLTRAPDSPSSVGRPSSDPGLSPTQPCPEGSGDCRKQCE |
| member 8 isoform | PDYYLDEAGRCTACVSCSRDDLVEKTPCAWNSSRTCECRPGMICATSATNSCARCVPYPIC |
| 1 precursor | AAETVTKPQDMAEKDTTFEAPPLGTQPDCNPTPENGEAPASTSPTQSLLVDSQASKTLPIPTS |
| (NP_001234.3) | APVALSSTGKPVLDAGPVLFWVILVLVVVVGSSAFLLCHRRACRKRIRQKLHLCYPVQTSQ |
| | PKLELVDSRPRRSSTQLRSGASVTEPVAEERGLMSQPLMETCHSVGAAYLESLPLQDASPA |
| | GGPSSPRDLPEPRVSTEHTNNKIEKIYIMKADTVIVGTVKAELPEGRGLAGPAEPELEEELEA |
| | DHTPHYPEQETEPPLGSCSDVMLSVEEEGKEDPLPTAASGK |
| 2055 | QVQLVQSGAEVKKPGASVKVSCKVSGYTFTDYYITWVRQAPGQALEWMGWIYPGSGNTK |
| heavy chain | YSQKFQGRFVFSVDTSASTAYLQISSLKAEDTAVYYCANYGNYWFAYWGQGTLVTVS |
| variable region | |
| (CD30L) | |
| 2056 | EIVLTQSPDSLAVSLGERATINCKASQSVDFDGDSYLNWYQQKPGQPPKVLIYAASTLQSGV |
| light chain | PSRFSGSGSGTDFTLTINSLEAEDAATYYCQQSNEDPWTFGGGTKVEIK |
| variable | |
| region (CD30L) | |
| 2057 | TCCTTATTCCTGGCTCTTCCAGAGGCTGGTGGCTCTTAGGGCTCAGCAGGGCTGCTGCCT |
| | GGGACAGGGAGACCAGGGGACCCAGGAAGCCCTGGCTCAGAGGAACTGCATTCTGCCC |
| | GCAACAAAGACGGAGTCAAACAAGAACTCCGGAGTTTGCAGAAGTGTTTCCCTTTAAG |
| | GAAACGCTTCTATCCTGTGCAGCAACCTAGGTTTAGGGTCCAACTGTCTCCAAAGGACC |
| | ACTTGAGAAGCAAGAAGGAGCCTGGTGAGGCTGCTCCCAGTGCCCTGCGGCCAGCAGT |
| | CCACACAGCCGGAGAGCCCATCTCTGAGCTGGGCGCCCCATGGCAAAGCACCCCCTGA |
| | ACTTACCAGCTCGAAGGCTGCTGAAGAGCAGGAAGAGCAGTCCAGGACTGGAAAAAA |
| | CAACCAGAAAGGCAGGTGAGGCGGGGCCACCAGGGAGTTACCTGTATCTTCCAAACAG |
| | GAACAGGCTGGTTCCCATGAGCAGGAGGTAGTTANGCCCATCACAGAAATGTGAAGGC |
| | AGAGCCAGGAGCCTGCCTGCCTCTGTTCTCCATGGGAATTCGGGGTC |
| | CGGGGCCAGGACCACCACCCTCCTGCCCAGCACAGCAAGTCCGAGGGGCTGGCAGCCT |
| | CTCACCACTCTCTAGCTCTCGCGGTAACAAATCCCCAGAAACACAGTGATGCTGGCGTG |
| | AGGATGAAGGGGAGCGAGGGACGCCAGTGAGGCTGGTCTAGCCTTCCTGGGGACAGAT |
| | GCCAAGAAGTTGTAGGAAATCAGAAAAAAAAAAAAAAAAAAAAGCTTTCAGGGCACA |
| | TATAGTAGGAGTGTCACTTAAAATGCTGGGGAGACGGAGAACATCAGGCGTGCCCGTG |
| | ACAGGGTCAGGTGAGTGACGGGACAGCCCTGTGCGGAACGCGGATCATTTTGAAACCA |
| | AACCTGAGCTCCTCCTGTTTCAGATCCTGCTGCAGCTGGTGCCCCTGCCTCGGGCAGCA |
| | AATTCCCCTCGTGACTCCAATGC |
| 2058 | TCTGGCTGCCGACAGTCAGGTTCTGCTGCAGAAGCACGTTCTCTATGCAGCAGCCAATG |
| | TTCACGCTGGGGGTCCGTGCGATCCTCAGCACGCTGACCACGTCATACAAGCCCCGCAT |
| | GTTCAAGAAGACGGTGTCATTCTGCAGAGCCTGGTCCAGCAGGCTGTTGTCCGTCTTAT |
| | TGATCCAGTACACGTTGGGCCTGGGGTAGCCGTTTATGGATGTACACGTGAAGGTGAGC |
| | TCATCCTGGGAGGGGCTGTGGGGGGCGCTGACGACGGGCACGCTGAAGTTTGCTGCAG |
| | GGGAGGGAAACAGATTGTGAGAGATGCCAGACCCTGCTGGTCAAGAAACAGAGGGTA |
| | CACGGTGCCAAGGCTGGGTCAGAGGGGAGGCGGCCCATTGTGCCCCGACATGGGTGAC |
| | AGGCCAGGACC |
| | AGGGCTGTGGTCCGAGCAGCAGGCTCCGCCCTGTCCTGCCCAAGAGTCATCCCCCAAGC |
| | CAGTCCCAGTAGCCAGGGACCNCTGAGCCTGTCGGGCAGTGACTGGCACCCACAGACC |
| | CCCCACTCCACAGCCGTCGGCTGTGCCGGGACCCCCTCATTTGGATCTGGTCATTCTCA |
| | CTGCTGACACAGGGGCTCACAGTCCATCTGAAATGGACACAGCTGTTCTCCCCAATGAA |
| | CTGCCCAGAGCTCCTTGGTTGCCTCTTATTTTTCTCTTAAATTTTGTTCTCATTTAGTGCT |
| | TTATAGAGTTCCCTTCAAATTTCACTTTTAAAACTCTAGTTTGGATGTGGTGGCTCACGC |
| | CTGTAATCACAGCACTTTGGGAGGGTGAGGCAGGAGGATCGCTTGTGTCCAGGAGTTT |
| | GAGACCAGCCTGGGCAACATAGTGAGACCCCATTTCTACAAATAATCAAAATTAGCCA |
| | GGCGAGGTGGCATGTGCCTGTAGTCCCAGATACTCAGGAGGCTGAAGCAGGAGAATTG |
| | CTTGAGCTCAGGAGGTCAAGGCTGCAGCGTGATCACGCTACTGCACTCCAG |
| 2059 | AGAATTTCTCAGCAATTACATTATATTGCTGCTCACTTGCAGTAGGAAAGCTGTCACAA |
| | ATGTGGCAGACACAGGGTAGCTTTACTGTTTGTGCAGCTACGTAAATGCCTGTGGGAAA |
| | ATCTTTTTTTTTGAGATGGAGTCTTGCTCCGTCACCCAGGCTGGGGTGCAATGGCGTGAT |
| | CTCAGCTCACTGCAACCCCCGACTCTGGGGTTCACGCCATTCTCCTGCCTCACCCTCCCA |
| | AGTAGCTGGGATTACAGGTGCCCGCCACCATGCCCAGCTAATTTTTTGTATTTTTAGTA |
| | GAGATCGGGCTTCACTATGTTGGCCAGCCTGGTCTCGAACTCCTGACCTCAGGTGATCC |
| | ACCCGCCTTGGCCTCCCAAAGTCCTGGGATTACAGGAGTGAGCCACTGCACCTGGCACC |
| | TGCAGGAAAATCTGTGTGTGTCCCCTGCCCCCAAAGCCCTCTGGGCTGGAAGGCTTGGC |
| | AAGGTTTCAGGTTTGTGCTGCGGTGCNTCGTCCTTATTCCTGGCTCTTCCAGAGGCTGGT |
| | GGCTCTTAGGGCTCAGCAGGGCTGCTGCCTGGGACAGGGAGACCAGGGGACCCAGGAA |
| | GCCCTGGCTCAGAGGAACTGCATTCTGCCCGCAACAAAGACGGAGTCAAACAAGAACT |
| | CCGGAGTTTGCAGAAGTGTTTCCCTTTAAGGAAACGCTTCTATCCTGTGCAGCAACCTA |
| | GGTTTAGGGTCCAACTGTCTCCAAAGGACCACTTGAGAAGCAAGAAGGAGCCTGGTGA |
| | GGCTGCTCCCAGTGCCCTGCGGCCAGCAGTCCACACAGCCGGAGAGCCCATCTCTGAG |
| | CTGGGCGCCCCATGGCAAAGCACCCCCTGAACTTACCAGCTCGAAGGCTGCTGAAGAG |
| | CAGGAAGAGCAGTCCAGGACTGGAAAAAACAACCAGAAAGGCAGGTGAGGCGGGGCC |
| | ACCAGGGAGTTACCTGTATCTTCCAAACAGGAACAGGCTGGTTCCCATGAGCAGGAGG |
| | TAG |

### 7.19 Example 19: Validation of an 8-SNP Model for TL1A Companion Diagnostics

The machine learning workflow identified several SNP model combinations for the development of the TL1A companion diagnostic (TL1A CDx). Previous analyses had identified 3-SNP combination models composed of variants associated with TNFSF15 (rs6478109), ICOSLG (rs7278257, rs2070557), ETS1 (rs7935393), and RBFOX1 (rs9806914) genes as well as variant rs1892231. Next, 8-SNP models were investigated to identify novel combinations with improved positive predictive value (PPV), as well as negative predictive value (NPV).

S-SNP models were identified using the methods described above via a Cedars Sinai Crohn's Disease cohort. These additional SNP models (Models_1-Model_495) consisted of 8-SNIP combinations of the SNPs selected from Table 27.

TABLE 27
Candidate SNPs for Building the 8-SNP Models

| SNPs | rs6478109 | rs56124762 | rs7935393 | rs9806914 | rs1892231 | rs16901748 | rs12934476 | rs2297437 |
|---|---|---|---|---|---|---|---|---|
| Related | TNFSF15 | ICOSLG | ETS1 | RBFOX1 | C14orf177 | CTNND2 | CLEC16A | RTEL1- |
| Genes | | | | | | | | TNFRSF6B |
| SEQ | 2039 | 2057 | 2004 | 2028 | 2026 | 2011 | 2005 | 2019 |
| ID NO | | | | | | | | |

| SNPs | rs1326860 | rs12457255 | rs2815844 | rs10974900 | rs2409750 | rs1541020 | rs4942248 | rs7759385 |
|---|---|---|---|---|---|---|---|---|
| Related | LINC01031 | PTPN2 | RGS7 | JAK2 | XKR6 | RBM17 | ENOX1 | PRDM1 |
| Genes | | | | | | | | |
| SEQ | 2024 | 2006 | 2012 | 2009 | 2014 | 2015 | 2016 | 2032 |
| ID NO | | | | | | | | |

Table 25 provides, in accordance with the embodiments disclosed herein, the 8-SNP model combinations.

TABLE 25
8-SNP Models

| | |
|---|---|
| Model_1 | rs6478109, rs56124762, rs1892231, rs16901748, rs12934476, rs7935393, rs9806914, rs2297437 |
| Model_2 | rs6478109, rs56124762, rs1892231, rs16901748, rs12934476, rs7935393, rs9806914, rs1326860 |
| Model_3 | rs6478109, rs56124762, rs1892231, rs16901748, rs12934476, rs7935393, rs9806914, rs12457255 |
| Model_4 | rs6478109, rs56124762, rs1892231, rs16901748, rs12934476, rs7935393, rs9806914, rs2815844 |
| Model_5 | rs6478109, rs56124762, rs1892231, rs16901748, rs12934476, rs7935393, rs9806914, rs10974900 |
| Model_6 | rs6478109, rs56124762, rs1892231, rs16901748, rs12934476, rs7935393, rs9806914, rs2409750 |
| Model_7 | rs6478109, rs56124762, rs1892231, rs16901748, rs12934476, rs7935393, rs9806914, rs1541020 |
| Model_8 | rs6478109, rs56124762, rs1892231, rs16901748, rs12934476, rs7935393, rs9806914, rs4942248 |
| Model_9 | rs6478109, rs56124762, rs1892231, rs16901748, rs12934476, rs7935393, rs9806914, rs7759385 |
| Model_10 | rs6478109, rs56124762, rs1892231, rs16901748, rs12934476, rs7935393, rs2297437, rs1326860 |
| Model_11 | rs6478109, rs56124762, rs1892231, rs16901748, rs12934476, rs7935393, rs2297437, rs12457255 |
| Model_12 | rs6478109, rs56124762, rs1892231, rs16901748, rs12934476, rs7935393, rs2297437, rs2815844 |
| Model_13 | rs6478109, rs56124762, rs1892231, rs16901748, rs12934476, rs7935393, rs2297437, rs10974900 |
| Model_14 | rs6478109, rs56124762, rs1892231, rs16901748, rs12934476, rs7935393, rs2297437, rs2409750 |
| Model_15 | rs6478109, rs56124762, rs1892231, rs16901748, rs12934476, rs7935393, rs2297437, rs1541020 |
| Model_16 | rs6478109, rs56124762, rs1892231, rs16901748, rs12934476, rs7935393, rs2297437, rs4942248 |
| Model_17 | rs6478109, rs56124762, rs1892231, rs16901748, rs12934476, rs7935393, rs2297437, rs7759385 |
| Model_18 | rs6478109, rs56124762, rs1892231, rs16901748, rs12934476, rs7935393, rs1326860, rs12457255 |
| Model_19 | rs6478109, rs56124762, rs1892231, rs16901748, rs12934476, rs7935393, rs1326860, rs2815844 |
| Model_20 | rs6478109, rs56124762, rs1892231, rs16901748, rs12934476, rs7935393, rs1326860, rs10974900 |
| Model_21 | rs6478109, rs56124762, rs1892231, rs16901748, rs12934476, rs7935393, rs1326860, rs2409750 |
| Model_22 | rs6478109, rs56124762, rs1892231, rs16901748, rs12934476, rs7935393, rs1326860, rs1541020 |
| Model_23 | rs6478109, rs56124762, rs1892231, rs16901748, rs12934476, rs7935393, rs1326860, rs4942248 |
| Model_24 | rs6478109, rs56124762, rs1892231, rs16901748, rs12934476, rs7935393, rs1326860, rs7759385 |
| Model_25 | rs6478109, rs56124762, rs1892231, rs16901748, rs12934476, rs7935393, rs12457255, rs2815844 |
| Model_26 | rs6478109, rs56124762, rs1892231, rs16901748, rs12934476, rs7935393, rs12457255, rs10974900 |
| Model_27 | rs6478109, rs56124762, rs1892231, rs16901748, rs12934476, rs7935393, rs12457255, rs2409750 |
| Model_28 | rs6478109, rs56124762, rs1892231, rs16901748, rs12934476, rs7935393, rs12457255, rs1541020 |
| Model_29 | rs6478109, rs56124762, rs1892231, rs16901748, rs12934476, rs7935393, rs12457255, rs4942248 |
| Model_30 | rs6478109, rs56124762, rs1892231, rs16901748, rs12934476, rs7935393, rs12457255, rs7759385 |
| Model_31 | rs6478109, rs56124762, rs1892231, rs16901748, rs12934476, rs7935393, rs2815844, rs10974900 |
| Model_32 | rs6478109, rs56124762, rs1892231, rs16901748, rs12934476, rs7935393, rs2815844, rs2409750 |
| Model_33 | rs6478109, rs56124762, rs1892231, rs16901748, rs12934476, rs7935393, rs2815844, rs1541020 |
| Model_34 | rs6478109, rs56124762, rs1892231, rs16901748, rs12934476, rs7935393, rs2815844, rs4942248 |
| Model_35 | rs6478109, rs56124762, rs1892231, rs16901748, rs12934476, rs7935393, rs2815844, rs7759385 |
| Model_36 | rs6478109, rs56124762, rs1892231, rs16901748, rs12934476, rs7935393, rs10974900, rs2409750 |
| Model_37 | rs6478109, rs56124762, rs1892231, rs16901748, rs12934476, rs7935393, rs10974900, rs1541020 |
| Model_38 | rs6478109, rs56124762, rs1892231, rs16901748, rs12934476, rs7935393, rs10974900, rs4942248 |
| Model_39 | rs6478109, rs56124762, rs1892231, rs16901748, rs12934476, rs7935393, rs10974900, rs7759385 |
| Model_40 | rs6478109, rs56124762, rs1892231, rs16901748, rs12934476, rs7935393, rs2409750, rs1541020 |
| Model_41 | rs6478109, rs56124762, rs1892231, rs16901748, rs12934476, rs7935393, rs2409750, rs4942248 |
| Model_42 | rs6478109, rs56124762, rs1892231, rs16901748, rs12934476, rs7935393, rs2409750, rs7759385 |
| Model_43 | rs6478109, rs56124762, rs1892231, rs16901748, rs12934476, rs7935393, rs1541020, rs4942248 |
| Model_44 | rs6478109, rs56124762, rs1892231, rs16901748, rs12934476, rs7935393, rs1541020, rs7759385 |
| Model_45 | rs6478109, rs56124762, rs1892231, rs16901748, rs12934476, rs7935393, rs4942248, rs7759385 |
| Model_46 | rs6478109, rs56124762, rs1892231, rs16901748, rs12934476, rs9806914, rs2297437, rs1326860 |
| Model_47 | rs6478109, rs56124762, rs1892231, rs16901748, rs12934476, rs9806914, rs2297437, rs12457255 |
| Model_48 | rs6478109, rs56124762, rs1892231, rs16901748, rs12934476, rs9806914, rs2297437, rs2815844 |
| Model_49 | rs6478109, rs56124762, rs1892231, rs16901748, rs12934476, rs9806914, rs2297437, rs10974900 |
| Model_50 | rs6478109, rs56124762, rs1892231, rs16901748, rs12934476, rs9806914, rs2297437, rs2409750 |
| Model_51 | rs6478109, rs56124762, rs1892231, rs16901748, rs12934476, rs9806914, rs2297437, rs1541020 |
| Model_52 | rs6478109, rs56124762, rs1892231, rs16901748, rs12934476, rs9806914, rs2297437, rs4942248 |
| Model_53 | rs6478109, rs56124762, rs1892231, rs16901748, rs12934476, rs9806914, rs2297437, rs7759385 |
| Model_54 | rs6478109, rs56124762, rs1892231, rs16901748, rs12934476, rs9806914, rs1326860, rs12457255 |
| Model_55 | rs6478109, rs56124762, rs1892231, rs16901748, rs12934476, rs9806914, rs1326860, rs2815844 |
| Model_56 | rs6478109, rs56124762, rs1892231, rs16901748, rs12934476, rs9806914, rs1326860, rs10974900 |
| Model_57 | rs6478109, rs56124762, rs1892231, rs16901748, rs12934476, rs9806914, rs1326860, rs2409750 |
| Model_58 | rs6478109, rs56124762, rs1892231, rs16901748, rs12934476, rs9806914, rs1326860, rs1541020 |
| Model_59 | rs6478109, rs56124762, rs1892231, rs16901748, rs12934476, rs9806914, rs1326860, rs4942248 |
| Model_60 | rs6478109, rs56124762, rs1892231, rs16901748, rs12934476, rs9806914, rs1326860, rs7759385 |
| Model_61 | rs6478109, rs56124762, rs1892231, rs16901748, rs12934476, rs9806914, rs12457255, rs2815844 |
| Model_62 | rs6478109, rs56124762, rs1892231, rs16901748, rs12934476, rs9806914, rs12457255, rs10974900 |
| Model_63 | rs6478109, rs56124762, rs1892231, rs16901748, rs12934476, rs9806914, rs12457255, rs2409750 |
| Model_64 | rs6478109, rs56124762, rs1892231, rs16901748, rs12934476, rs9806914, rs12457255, rs1541020 |
| Model_65 | rs6478109, rs56124762, rs1892231, rs16901748, rs12934476, rs9806914, rs12457255, rs4942248 |
| Model_66 | rs6478109, rs56124762, rs1892231, rs16901748, rs12934476, rs9806914, rs12457255, rs7759385 |
| Model_67 | rs6478109, rs56124762, rs1892231, rs16901748, rs12934476, rs9806914, rs2815844, rs10974900 |
| Model_68 | rs6478109, rs56124762, rs1892231, rs16901748, rs12934476, rs9806914, rs2815844, rs2409750 |
| Model_69 | rs6478109, rs56124762, rs1892231, rs16901748, rs12934476, rs9806914, rs2815844, rs1541020 |
| Model_70 | rs6478109, rs56124762, rs1892231, rs16901748, rs12934476, rs9806914, rs2815844, rs4942248 |
| Model_71 | rs6478109, rs56124762, rs1892231, rs16901748, rs12934476, rs9806914, rs2815844, rs7759385 |
| Model_72 | rs6478109, rs56124762, rs1892231, rs16901748, rs12934476, rs9806914, rs10974900, rs2409750 |
| Model_73 | rs6478109, rs56124762, rs1892231, rs16901748, rs12934476, rs9806914, rs10974900, rs1541020 |
| Model_74 | rs6478109, rs56124762, rs1892231, rs16901748, rs12934476, rs9806914, rs10974900, rs4942248 |
| Model_75 | rs6478109, rs56124762, rs1892231, rs16901748, rs12934476, rs9806914, rs10974900, rs7759385 |
| Model_76 | rs6478109, rs56124762, rs1892231, rs16901748, rs12934476, rs9806914, rs2409750, rs1541020 |
| Model_77 | rs6478109, rs56124762, rs1892231, rs16901748, rs12934476, rs9806914, rs2409750, rs4942248 |
| Model_78 | rs6478109, rs56124762, rs1892231, rs16901748, rs12934476, rs9806914, rs2409750, rs7759385 |
| Model_79 | rs6478109, rs56124762, rs1892231, rs16901748, rs12934476, rs9806914, rs1541020, rs4942248 |
| Model_80 | rs6478109, rs56124762, rs1892231, rs16901748, rs12934476, rs9806914, rs1541020, rs7759385 |
| Model_81 | rs6478109, rs56124762, rs1892231, rs16901748, rs12934476, rs9806914, rs4942248, rs7759385 |
| Model_82 | rs6478109, rs56124762, rs1892231, rs16901748, rs12934476, rs2297437, rs1326860, rs12457255 |
| Model_83 | rs6478109, rs56124762, rs1892231, rs16901748, rs12934476, rs2297437, rs1326860, rs2815844 |
| Model_84 | rs6478109, rs56124762, rs1892231, rs16901748, rs12934476, rs2297437, rs1326860, rs10974900 |
| Model_85 | rs6478109, rs56124762, rs1892231, rs16901748, rs12934476, rs2297437, rs1326860, rs2409750 |
| Model_86 | rs6478109, rs56124762, rs1892231, rs16901748, rs12934476, rs2297437, rs1326860, rs1541020 |
| Model_87 | rs6478109, rs56124762, rs1892231, rs16901748, rs12934476, rs2297437, rs1326860, rs4942248 |
| Model_88 | rs6478109, rs56124762, rs1892231, rs16901748, rs12934476, rs2297437, rs1326860, rs7759385 |
| Model_89 | rs6478109, rs56124762, rs1892231, rs16901748, rs12934476, rs2297437, rs12457255, rs2815844 |
| Model_90 | rs6478109, rs56124762, rs1892231, rs16901748, rs12934476, rs2297437, rs12457255, rs10974900 |
| Model_91 | rs6478109, rs56124762, rs1892231, rs16901748, rs12934476, rs2297437, rs12457255, rs2409750 |
| Model_92 | rs6478109, rs56124762, rs1892231, rs16901748, rs12934476, rs2297437, rs12457255, rs1541020 |
| Model_93 | rs6478109, rs56124762, rs1892231, rs16901748, rs12934476, rs2297437, rs12457255, rs4942248 |
| Model_94 | rs6478109, rs56124762, rs1892231, rs16901748, rs12934476, rs2297437, rs12457255, rs7759385 |
| Model_95 | rs6478109, rs56124762, rs1892231, rs16901748, rs12934476, rs2297437, rs2815844, rs10974900 |
| Model_96 | rs6478109, rs56124762, rs1892231, rs16901748, rs12934476, rs2297437, rs2815844, rs2409750 |
| Model_97 | rs6478109, rs56124762, rs1892231, rs16901748, rs12934476, rs2297437, rs2815844, rs1541020 |
| Model_98 | rs6478109, rs56124762, rs1892231, rs16901748, rs12934476, rs2297437, rs2815844, rs4942248 |
| Model_99 | rs6478109, rs56124762, rs1892231, rs16901748, rs12934476, rs2297437, rs2815844, rs7759385 |
| Model_100 | rs6478109, rs56124762, rs1892231, rs16901748, rs12934476, rs2297437, rs10974900, rs2409750 |
| Model_101 | rs6478109, rs56124762, rs1892231, rs16901748, rs12934476, rs2297437, rs10974900, rs1541020 |
| Model_102 | rs6478109, rs56124762, rs1892231, rs16901748, rs12934476, rs2297437, rs10974900, rs4942248 |
| Model_103 | rs6478109, rs56124762, rs1892231, rs16901748, rs12934476, rs2297437, rs10974900, rs7759385 |
| Model_104 | rs6478109, rs56124762, rs1892231, rs16901748, rs12934476, rs2297437, rs2409750, rs1541020 |
| Model_105 | rs6478109, rs56124762, rs1892231, rs16901748, rs12934476, rs2297437, rs2409750, rs4942248 |
| Model_106 | rs6478109, rs56124762, rs1892231, rs16901748, rs12934476, rs2297437, rs2409750, rs7759385 |
| Model_107 | rs6478109, rs56124762, rs1892231, rs16901748, rs12934476, rs2297437, rs1541020, rs4942248 |
| Model_108 | rs6478109, rs56124762, rs1892231, rs16901748, rs12934476, rs2297437, rs1541020, rs7759385 |
| Model_109 | rs6478109, rs56124762, rs1892231, rs16901748, rs12934476, rs2297437, rs4942248, rs7759385 |
| Model_110 | rs6478109, rs56124762, rs1892231, rs16901748, rs12934476, rs1326860, rs12457255, rs2815844 |
| Model_111 | rs6478109, rs56124762, rs1892231, rs16901748, rs12934476, rs1326860, rs12457255, rs10974900 |
| Model_112 | rs6478109, rs56124762, rs1892231, rs16901748, rs12934476, rs1326860, rs12457255, rs2409750 |
| Model_113 | rs6478109, rs56124762, rs1892231, rs16901748, rs12934476, rs1326860, rs12457255, rs1541020 |
| Model_114 | rs6478109, rs56124762, rs1892231, rs16901748, rs12934476, rs1326860, rs12457255, rs4942248 |
| Model_115 | rs6478109, rs56124762, rs1892231, rs16901748, rs12934476, rs1326860, rs12457255, rs7759385 |
| Model_116 | rs6478109, rs56124762, rs1892231, rs16901748, rs12934476, rs1326860, rs2815844, rs10974900 |
| Model_117 | rs6478109, rs56124762, rs1892231, rs16901748, rs12934476, rs1326860, rs2815844, rs2409750 |
| Model_118 | rs6478109, rs56124762, rs1892231, rs16901748, rs12934476, rs1326860, rs2815844, rs1541020 |
| Model_119 | rs6478109, rs56124762, rs1892231, rs16901748, rs12934476, rs1326860, rs2815844, rs4942248 |
| Model_120 | rs6478109, rs56124762, rs1892231, rs16901748, rs12934476, rs1326860, rs2815844, rs7759385 |
| Model_121 | rs6478109, rs56124762, rs1892231, rs16901748, rs12934476, rs1326860, rs10974900, rs2409750 |
| Model_122 | rs6478109, rs56124762, rs1892231, rs16901748, rs12934476, rs1326860, rs10974900, rs1541020 |
| Model_123 | rs6478109, rs56124762, rs1892231, rs16901748, rs12934476, rs1326860, rs10974900, rs4942248 |
| Model_124 | rs6478109, rs56124762, rs1892231, rs16901748, rs12934476, rs1326860, rs10974900, rs7759385 |
| Model_125 | rs6478109, rs56124762, rs1892231, rs16901748, rs12934476, rs1326860, rs2409750, rs1541020 |
| Model_126 | rs6478109, rs56124762, rs1892231, rs16901748, rs12934476, rs1326860, rs2409750, rs4942248 |
| Model_127 | rs6478109, rs56124762, rs1892231, rs16901748, rs12934476, rs1326860, rs2409750, rs7759385 |
| Model_128 | rs6478109, rs56124762, rs1892231, rs16901748, rs12934476, rs1326860, rs1541020, rs4942248 |
| Model_129 | rs6478109, rs56124762, rs1892231, rs16901748, rs12934476, rs1326860, rs1541020, rs7759385 |
| Model_130 | rs6478109, rs56124762, rs1892231, rs16901748, rs12934476, rs1326860, rs4942248, rs7759385 |
| Model_131 | rs6478109, rs56124762, rs1892231, rs16901748, rs12934476, rs12457255, rs2815844, rs10974900 |
| Model_132 | rs6478109, rs56124762, rs1892231, rs16901748, rs12934476, rs12457255, rs2815844, rs2409750 |
| Model_133 | rs6478109, rs56124762, rs1892231, rs16901748, rs12934476, rs12457255, rs2815844, rs1541020 |
| Model_134 | rs6478109, rs56124762, rs1892231, rs16901748, rs12934476, rs12457255, rs2815844, rs4942248 |
| Model_135 | rs6478109, rs56124762, rs1892231, rs16901748, rs12934476, rs12457255, rs2815844, rs7759385 |
| Model_136 | rs6478109, rs56124762, rs1892231, rs16901748, rs12934476, rs12457255, rs10974900, rs2409750 |
| Model_137 | rs6478109, rs56124762, rs1892231, rs16901748, rs12934476, rs12457255, rs10974900, rs1541020 |
| Model_138 | rs6478109, rs56124762, rs1892231, rs16901748, rs12934476, rs12457255, rs10974900, rs4942248 |
| Model_139 | rs6478109, rs56124762, rs1892231, rs16901748, rs12934476, rs12457255, rs10974900, rs7759385 |
| Model_140 | rs6478109, rs56124762, rs1892231, rs16901748, rs12934476, rs12457255, rs2409750, rs1541020 |
| Model_141 | rs6478109, rs56124762, rs1892231, rs16901748, rs12934476, rs12457255, rs2409750, rs4942248 |
| Model_142 | rs6478109, rs56124762, rs1892231, rs16901748, rs12934476, rs12457255, rs2409750, rs7759385 |
| Model_143 | rs6478109, rs56124762, rs1892231, rs16901748, rs12934476, rs12457255, rs1541020, rs4942248 |
| Model_144 | rs6478109, rs56124762, rs1892231, rs16901748, rs12934476, rs12457255, rs1541020, rs7759385 |
| Model_145 | rs6478109, rs56124762, rs1892231, rs16901748, rs12934476, rs12457255, rs4942248, rs7759385 |
| Model_146 | rs6478109, rs56124762, rs1892231, rs16901748, rs12934476, rs2815844, rs10974900, rs2409750 |
| Model_147 | rs6478109, rs56124762, rs1892231, rs16901748, rs12934476, rs2815844, rs10974900, rs1541020 |
| Model_148 | rs6478109, rs56124762, rs1892231, rs16901748, rs12934476, rs2815844, rs10974900, rs4942248 |
| Model_149 | rs6478109, rs56124762, rs1892231, rs16901748, rs12934476, rs2815844, rs10974900, rs7759385 |
| Model_150 | rs6478109, rs56124762, rs1892231, rs16901748, rs12934476, rs2815844, rs2409750, rs1541020 |
| Model_151 | rs6478109, rs56124762, rs1892231, rs16901748, rs12934476, rs2815844, rs2409750, rs4942248 |
| Model_152 | rs6478109, rs56124762, rs1892231, rs16901748, rs12934476, rs2815844, rs2409750, rs7759385 |
| Model_153 | rs6478109, rs56124762, rs1892231, rs16901748, rs12934476, rs2815844, rs1541020, rs4942248 |
| Model_154 | rs6478109, rs56124762, rs1892231, rs16901748, rs12934476, rs2815844, rs1541020, rs7759385 |
| Model_155 | rs6478109, rs56124762, rs1892231, rs16901748, rs12934476, rs2815844, rs4942248, rs7759385 |
| Model_156 | rs6478109, rs56124762, rs1892231, rs16901748, rs12934476, rs10974900, rs2409750, rs1541020 |
| Model_157 | rs6478109, rs56124762, rs1892231, rs16901748, rs12934476, rs10974900, rs2409750, rs4942248 |
| Model_158 | rs6478109, rs56124762, rs1892231, rs16901748, rs12934476, rs10974900, rs2409750, rs7759385 |
| Model_159 | rs6478109, rs56124762, rs1892231, rs16901748, rs12934476, rs10974900, rs1541020, rs4942248 |
| Model_160 | rs6478109, rs56124762, rs1892231, rs16901748, rs12934476, rs10974900, rs1541020, rs7759385 |
| Model_161 | rs6478109, rs56124762, rs1892231, rs16901748, rs12934476, rs10974900, rs4942248, rs7759385 |
| Model_162 | rs6478109, rs56124762, rs1892231, rs16901748, rs12934476, rs2409750, rs1541020, rs4942248 |
| Model_163 | rs6478109, rs56124762, rs1892231, rs16901748, rs12934476, rs2409750, rs1541020, rs7759385 |
| Model_164 | rs6478109, rs56124762, rs1892231, rs16901748, rs12934476, rs2409750, rs4942248, rs7759385 |
| Model_165 | rs6478109, rs56124762, rs1892231, rs16901748, rs12934476, rs1541020, rs4942248, rs7759385 |
| Model_166 | rs6478109, rs56124762, rs1892231, rs16901748, rs7935393, rs9806914, rs2297437, rs1326860 |
| Model_167 | rs6478109, rs56124762, rs1892231, rs16901748, rs7935393, rs9806914, rs2297437, rs12457255 |
| Model_168 | rs6478109, rs56124762, rs1892231, rs16901748, rs7935393, rs9806914, rs2297437, rs2815844 |
| Model_169 | rs6478109, rs56124762, rs1892231, rs16901748, rs7935393, rs9806914, rs2297437, rs10974900 |
| Model_170 | rs6478109, rs56124762, rs1892231, rs16901748, rs7935393, rs9806914, rs2297437, rs2409750 |
| Model_171 | rs6478109, rs56124762, rs1892231, rs16901748, rs7935393, rs9806914, rs2297437, rs1541020 |
| Model_172 | rs6478109, rs56124762, rs1892231, rs16901748, rs7935393, rs9806914, rs2297437, rs4942248 |
| Model_173 | rs6478109, rs56124762, rs1892231, rs16901748, rs7935393, rs9806914, rs2297437, rs7759385 |
| Model_174 | rs6478109, rs56124762, rs1892231, rs16901748, rs7935393, rs9806914, rs1326860, rs12457255 |
| Model_175 | rs6478109, rs56124762, rs1892231, rs16901748, rs7935393, rs9806914, rs1326860, rs2815844 |
| Model_176 | rs6478109, rs56124762, rs1892231, rs16901748, rs7935393, rs9806914, rs1326860, rs10974900 |
| Model_177 | rs6478109, rs56124762, rs1892231, rs16901748, rs7935393, rs9806914, rs1326860, rs2409750 |
| Model_178 | rs6478109, rs56124762, rs1892231, rs16901748, rs7935393, rs9806914, rs1326860, rs1541020 |
| Model_179 | rs6478109, rs56124762, rs1892231, rs16901748, rs7935393, rs9806914, rs1326860, rs4942248 |
| Model_180 | rs6478109, rs56124762, rs1892231, rs16901748, rs7935393, rs9806914, rs1326860, rs7759385 |
| Model_181 | rs6478109, rs56124762, rs1892231, rs16901748, rs7935393, rs9806914, rs12457255, rs2815844 |
| Model_182 | rs6478109, rs56124762, rs1892231, rs16901748, rs7935393, rs9806914, rs12457255, rs10974900 |
| Model_183 | rs6478109, rs56124762, rs1892231, rs16901748, rs7935393, rs9806914, rs12457255, rs2409750 |
| Model_184 | rs6478109, rs56124762, rs1892231, rs16901748, rs7935393, rs9806914, rs12457255, rs1541020 |
| Model_185 | rs6478109, rs56124762, rs1892231, rs16901748, rs7935393, rs9806914, rs12457255, rs4942248 |
| Model_186 | rs6478109, rs56124762, rs1892231, rs16901748, rs7935393, rs9806914, rs12457255, rs7759385 |
| Model_187 | rs6478109, rs56124762, rs1892231, rs16901748, rs7935393, rs9806914, rs2815844, rs10974900 |
| Model_188 | rs6478109, rs56124762, rs1892231, rs16901748, rs7935393, rs9806914, rs2815844, rs2409750 |
| Model_189 | rs6478109, rs56124762, rs1892231, rs16901748, rs7935393, rs9806914, rs2815844, rs1541020 |
| Model_190 | rs6478109, rs56124762, rs1892231, rs16901748, rs7935393, rs9806914, rs2815844, rs4942248 |
| Model_191 | rs6478109, rs56124762, rs1892231, rs16901748, rs7935393, rs9806914, rs2815844, rs7759385 |
| Model_192 | rs6478109, rs56124762, rs1892231, rs16901748, rs7935393, rs9806914, rs10974900, rs2409750 |
| Model_193 | rs6478109, rs56124762, rs1892231, rs16901748, rs7935393, rs9806914, rs10974900, rs1541020 |
| Model_194 | rs6478109, rs56124762, rs1892231, rs16901748, rs7935393, rs9806914, rs10974900, rs4942248 |
| Model_195 | rs6478109, rs56124762, rs1892231, rs16901748, rs7935393, rs9806914, rs10974900, rs7759385 |
| Model_196 | rs6478109, rs56124762, rs1892231, rs16901748, rs7935393, rs9806914, rs2409750, rs1541020 |
| Model_197 | rs6478109, rs56124762, rs1892231, rs16901748, rs7935393, rs9806914, rs2409750, rs4942248 |
| Model_198 | rs6478109, rs56124762, rs1892231, rs16901748, rs7935393, rs9806914, rs2409750, rs7759385 |
| Model_199 | rs6478109, rs56124762, rs1892231, rs16901748, rs7935393, rs9806914, rs1541020, rs4942248 |
| Model_200 | rs6478109, rs56124762, rs1892231, rs16901748, rs7935393, rs9806914, rs1541020, rs7759385 |
| Model_201 | rs6478109, rs56124762, rs1892231, rs16901748, rs7935393, rs9806914, rs4942248, rs7759385 |
| Model_202 | rs6478109, rs56124762, rs1892231, rs16901748, rs7935393, rs2297437, rs1326860, rs12457255 |
| Model_203 | rs6478109, rs56124762, rs1892231, rs16901748, rs7935393, rs2297437, rs1326860, rs2815844 |
| Model_204 | rs6478109, rs56124762, rs1892231, rs16901748, rs7935393, rs2297437, rs1326860, rs10974900 |
| Model_205 | rs6478109, rs56124762, rs1892231, rs16901748, rs7935393, rs2297437, rs1326860, rs2409750 |
| Model_206 | rs6478109, rs56124762, rs1892231, rs16901748, rs7935393, rs2297437, rs1326860, rs1541020 |
| Model_207 | rs6478109, rs56124762, rs1892231, rs16901748, rs7935393, rs2297437, rs1326860, rs4942248 |
| Model_208 | rs6478109, rs56124762, rs1892231, rs16901748, rs7935393, rs2297437, rs1326860, rs7759385 |
| Model_209 | rs6478109, rs56124762, rs1892231, rs16901748, rs7935393, rs2297437, rs12457255, rs2815844 |
| Model_210 | rs6478109, rs56124762, rs1892231, rs16901748, rs7935393, rs2297437, rs12457255, rs10974900 |
| Model_211 | rs6478109, rs56124762, rs1892231, rs16901748, rs7935393, rs2297437, rs12457255, rs2409750 |
| Model_212 | rs6478109, rs56124762, rs1892231, rs16901748, rs7935393, rs2297437, rs12457255, rs1541020 |
| Model_213 | rs6478109, rs56124762, rs1892231, rs16901748, rs7935393, rs2297437, rs12457255, rs4942248 |
| Model_214 | rs6478109, rs56124762, rs1892231, rs16901748, rs7935393, rs2297437, rs12457255, rs7759385 |
| Model_215 | rs6478109, rs56124762, rs1892231, rs16901748, rs7935393, rs2297437, rs2815844, rs10974900 |
| Model_216 | rs6478109, rs56124762, rs1892231, rs16901748, rs7935393, rs2297437, rs2815844, rs2409750 |
| Model_217 | rs6478109, rs56124762, rs1892231, rs16901748, rs7935393, rs2297437, rs2815844, rs1541020 |
| Model_218 | rs6478109, rs56124762, rs1892231, rs16901748, rs7935393, rs2297437, rs2815844, rs4942248 |
| Model_219 | rs6478109, rs56124762, rs1892231, rs16901748, rs7935393, rs2297437, rs2815844, rs7759385 |
| Model_220 | rs6478109, rs56124762, rs1892231, rs16901748, rs7935393, rs2297437, rs10974900, rs2409750 |
| Model_221 | rs6478109, rs56124762, rs1892231, rs16901748, rs7935393, rs2297437, rs10974900, rs1541020 |
| Model_222 | rs6478109, rs56124762, rs1892231, rs16901748, rs7935393, rs2297437, rs10974900, rs4942248 |
| Model_223 | rs6478109, rs56124762, rs1892231, rs16901748, rs7935393, rs2297437, rs10974900, rs7759385 |
| Model_224 | rs6478109, rs56124762, rs1892231, rs16901748, rs7935393, rs2297437, rs2409750, rs1541020 |
| Model_225 | rs6478109, rs56124762, rs1892231, rs16901748, rs7935393, rs2297437, rs2409750, rs4942248 |
| Model_226 | rs6478109, rs56124762, rs1892231, rs16901748, rs7935393, rs2297437, rs2409750, rs7759385 |
| Model_227 | rs6478109, rs56124762, rs1892231, rs16901748, rs7935393, rs2297437, rs1541020, rs4942248 |
| Model_228 | rs6478109, rs56124762, rs1892231, rs16901748, rs7935393, rs2297437, rs1541020, rs7759385 |
| Model_229 | rs6478109, rs56124762, rs1892231, rs16901748, rs7935393, rs2297437, rs4942248, rs7759385 |
| Model_230 | rs6478109, rs56124762, rs1892231, rs16901748, rs7935393, rs1326860, rs12457255, rs2815844 |
| Model_231 | rs6478109, rs56124762, rs1892231, rs16901748, rs7935393, rs1326860, rs12457255, rs10974900 |
| Model_232 | rs6478109, rs56124762, rs1892231, rs16901748, rs7935393, rs1326860, rs12457255, rs2409750 |
| Model_233 | rs6478109, rs56124762, rs1892231, rs16901748, rs7935393, rs1326860, rs12457255, rs1541020 |
| Model_234 | rs6478109, rs56124762, rs1892231, rs16901748, rs7935393, rs1326860, rs12457255, rs4942248 |
| Model_235 | rs6478109, rs56124762, rs1892231, rs16901748, rs7935393, rs1326860, rs12457255, rs7759385 |
| Model_236 | rs6478109, rs56124762, rs1892231, rs16901748, rs7935393, rs1326860, rs2815844, rs10974900 |
| Model_237 | rs6478109, rs56124762, rs1892231, rs16901748, rs7935393, rs1326860, rs2815844, rs2409750 |
| Model_238 | rs6478109, rs56124762, rs1892231, rs16901748, rs7935393, rs1326860, rs2815844, rs1541020 |
| Model_239 | rs6478109, rs56124762, rs1892231, rs16901748, rs7935393, rs1326860, rs2815844, rs4942248 |
| Model_240 | rs6478109, rs56124762, rs1892231, rs16901748, rs7935393, rs1326860, rs2815844, rs7759385 |
| Model_241 | rs6478109, rs56124762, rs1892231, rs16901748, rs7935393, rs1326860, rs10974900, rs2409750 |
| Model_242 | rs6478109, rs56124762, rs1892231, rs16901748, rs7935393, rs1326860, rs10974900, rs1541020 |
| Model_243 | rs6478109, rs56124762, rs1892231, rs16901748, rs7935393, rs1326860, rs10974900, rs4942248 |
| Model_244 | rs6478109, rs56124762, rs1892231, rs16901748, rs7935393, rs1326860, rs10974900, rs7759385 |
| Model_245 | rs6478109, rs56124762, rs1892231, rs16901748, rs7935393, rs1326860, rs2409750, rs1541020 |
| Model_246 | rs6478109, rs56124762, rs1892231, rs16901748, rs7935393, rs1326860, rs2409750, rs4942248 |
| Model_247 | rs6478109, rs56124762, rs1892231, rs16901748, rs7935393, rs1326860, rs2409750, rs7759385 |
| Model_248 | rs6478109, rs56124762, rs1892231, rs16901748, rs7935393, rs1326860, rs1541020, rs4942248 |
| Model_249 | rs6478109, rs56124762, rs1892231, rs16901748, rs7935393, rs1326860, rs1541020, rs7759385 |
| Model_250 | rs6478109, rs56124762, rs1892231, rs16901748, rs7935393, rs1326860, rs4942248, rs7759385 |
| Model_251 | rs6478109, rs56124762, rs1892231, rs16901748, rs7935393, rs12457255, rs2815844, rs10974900 |
| Model_252 | rs6478109, rs56124762, rs1892231, rs16901748, rs7935393, rs12457255, rs2815844, rs2409750 |
| Model_253 | rs6478109, rs56124762, rs1892231, rs16901748, rs7935393, rs12457255, rs2815844, rs1541020 |
| Model_254 | rs6478109, rs56124762, rs1892231, rs16901748, rs7935393, rs12457255, rs2815844, rs4942248 |
| Model_255 | rs6478109, rs56124762, rs1892231, rs16901748, rs7935393, rs12457255, rs2815844, rs7759385 |
| Model_256 | rs6478109, rs56124762, rs1892231, rs16901748, rs7935393, rs12457255, rs10974900, rs2409750 |
| Model_257 | rs6478109, rs56124762, rs1892231, rs16901748, rs7935393, rs12457255, rs10974900, rs1541020 |
| Model_258 | rs6478109, rs56124762, rs1892231, rs16901748, rs7935393, rs12457255, rs10974900, rs4942248 |
| Model_259 | rs6478109, rs56124762, rs1892231, rs16901748, rs7935393, rs12457255, rs10974900, rs7759385 |
| Model_260 | rs6478109, rs56124762, rs1892231, rs16901748, rs7935393, rs12457255, rs2409750, rs1541020 |
| Model_261 | rs6478109, rs56124762, rs1892231, rs16901748, rs7935393, rs12457255, rs2409750, rs4942248 |
| Model_262 | rs6478109, rs56124762, rs1892231, rs16901748, rs7935393, rs12457255, rs2409750, rs7759385 |
| Model_263 | rs6478109, rs56124762, rs1892231, rs16901748, rs7935393, rs12457255, rs1541020, rs4942248 |
| Model_264 | rs6478109, rs56124762, rs1892231, rs16901748, rs7935393, rs12457255, rs1541020, rs7759385 |
| Model_265 | rs6478109, rs56124762, rs1892231, rs16901748, rs7935393, rs12457255, rs4942248, rs7759385 |
| Model_266 | rs6478109, rs56124762, rs1892231, rs16901748, rs7935393, rs2815844, rs10974900, rs2409750 |
| Model_267 | rs6478109, rs56124762, rs1892231, rs16901748, rs7935393, rs2815844, rs10974900, rs1541020 |
| Model_268 | rs6478109, rs56124762, rs1892231, rs16901748, rs7935393, rs2815844, rs10974900, rs4942248 |
| Model_269 | rs6478109, rs56124762, rs1892231, rs16901748, rs7935393, rs2815844, rs10974900, rs7759385 |
| Model_270 | rs6478109, rs56124762, rs1892231, rs16901748, rs7935393, rs2815844, rs2409750, rs1541020 |
| Model_271 | rs6478109, rs56124762, rs1892231, rs16901748, rs7935393, rs2815844, rs2409750, rs4942248 |
| Model_272 | rs6478109, rs56124762, rs1892231, rs16901748, rs7935393, rs2815844, rs2409750, rs7759385 |
| Model_273 | rs6478109, rs56124762, rs1892231, rs16901748, rs7935393, rs2815844, rs1541020, rs4942248 |
| Model_274 | rs6478109, rs56124762, rs1892231, rs16901748, rs7935393, rs2815844, rs1541020, rs7759385 |
| Model_275 | rs6478109, rs56124762, rs1892231, rs16901748, rs7935393, rs2815844, rs4942248, rs7759385 |
| Model_276 | rs6478109, rs56124762, rs1892231, rs16901748, rs7935393, rs10974900, rs2409750, rs1541020 |
| Model_277 | rs6478109, rs56124762, rs1892231, rs16901748, rs7935393, rs10974900, rs2409750, rs4942248 |
| Model_278 | rs6478109, rs56124762, rs1892231, rs16901748, rs7935393, rs10974900, rs2409750, rs7759385 |
| Model_279 | rs6478109, rs56124762, rs1892231, rs16901748, rs7935393, rs10974900, rs1541020, rs4942248 |
| Model_280 | rs6478109, rs56124762, rs1892231, rs16901748, rs7935393, rs10974900, rs1541020, rs7759385 |
| Model_281 | rs6478109, rs56124762, rs1892231, rs16901748, rs7935393, rs10974900, rs4942248, rs7759385 |
| Model_282 | rs6478109, rs56124762, rs1892231, rs16901748, rs7935393, rs2409750, rs1541020, rs4942248 |
| Model_283 | rs6478109, rs56124762, rs1892231, rs16901748, rs7935393, rs2409750, rs1541020, rs7759385 |
| Model_284 | rs6478109, rs56124762, rs1892231, rs16901748, rs7935393, rs2409750, rs4942248, rs7759385 |
| Model_285 | rs6478109, rs56124762, rs1892231, rs16901748, rs7935393, rs1541020, rs4942248, rs7759385 |
| Model_286 | rs6478109, rs56124762, rs1892231, rs16901748, rs9806914, rs2297437, rs1326860, rs12457255 |
| Model_287 | rs6478109, rs56124762, rs1892231, rs16901748, rs9806914, rs2297437, rs1326860, rs2815844 |
| Model_288 | rs6478109, rs56124762, rs1892231, rs16901748, rs9806914, rs2297437, rs1326860, rs10974900 |
| Model_289 | rs6478109, rs56124762, rs1892231, rs16901748, rs9806914, rs2297437, rs1326860, rs2409750 |
| Model_290 | rs6478109, rs56124762, rs1892231, rs16901748, rs9806914, rs2297437, rs1326860, rs1541020 |
| Model_291 | rs6478109, rs56124762, rs1892231, rs16901748, rs9806914, rs2297437, rs1326860, rs4942248 |
| Model_292 | rs6478109, rs56124762, rs1892231, rs16901748, rs9806914, rs2297437, rs1326860, rs7759385 |
| Model_293 | rs6478109, rs56124762, rs1892231, rs16901748, rs9806914, rs2297437, rs12457255, rs2815844 |
| Model_294 | rs6478109, rs56124762, rs1892231, rs16901748, rs9806914, rs2297437, rs12457255, rs10974900 |
| Model_295 | rs6478109, rs56124762, rs1892231, rs16901748, rs9806914, rs2297437, rs12457255, rs2409750 |
| Model_296 | rs6478109, rs56124762, rs1892231, rs16901748, rs9806914, rs2297437, rs12457255, rs1541020 |
| Model_297 | rs6478109, rs56124762, rs1892231, rs16901748, rs9806914, rs2297437, rs12457255, rs4942248 |
| Model_298 | rs6478109, rs56124762, rs1892231, rs16901748, rs9806914, rs2297437, rs12457255, rs7759385 |
| Model_299 | rs6478109, rs56124762, rs1892231, rs16901748, rs9806914, rs2297437, rs2815844, rs10974900 |
| Model_300 | rs6478109, rs56124762, rs1892231, rs16901748, rs9806914, rs2297437, rs2815844, rs2409750 |
| Model_301 | rs6478109, rs56124762, rs1892231, rs16901748, rs9806914, rs2297437, rs2815844, rs1541020 |
| Model_302 | rs6478109, rs56124762, rs1892231, rs16901748, rs9806914, rs2297437, rs2815844, rs4942248 |
| Model_303 | rs6478109, rs56124762, rs1892231, rs16901748, rs9806914, rs2297437, rs2815844, rs7759385 |
| Model_304 | rs6478109, rs56124762, rs1892231, rs16901748, rs9806914, rs2297437, rs10974900, rs2409750 |
| Model_305 | rs6478109, rs56124762, rs1892231, rs16901748, rs9806914, rs2297437, rs10974900, rs1541020 |
| Model_306 | rs6478109, rs56124762, rs1892231, rs16901748, rs9806914, rs2297437, rs10974900, rs4942248 |
| Model_307 | rs6478109, rs56124762, rs1892231, rs16901748, rs9806914, rs2297437, rs10974900, rs7759385 |
| Model_308 | rs6478109, rs56124762, rs1892231, rs16901748, rs9806914, rs2297437, rs2409750, rs1541020 |
| Model_309 | rs6478109, rs56124762, rs1892231, rs16901748, rs9806914, rs2297437, rs2409750, rs4942248 |
| Model_310 | rs6478109, rs56124762, rs1892231, rs16901748, rs9806914, rs2297437, rs2409750, rs7759385 |
| Model_311 | rs6478109, rs56124762, rs1892231, rs16901748, rs9806914, rs2297437, rs1541020, rs4942248 |
| Model_312 | rs6478109, rs56124762, rs1892231, rs16901748, rs9806914, rs2297437, rs1541020, rs7759385 |
| Model_313 | rs6478109, rs56124762, rs1892231, rs16901748, rs9806914, rs2297437, rs4942248, rs7759385 |
| Model_314 | rs6478109, rs56124762, rs1892231, rs16901748, rs9806914, rs1326860, rs12457255, rs2815844 |
| Model_315 | rs6478109, rs56124762, rs1892231, rs16901748, rs9806914, rs1326860, rs12457255, rs10974900 |
| Model_316 | rs6478109, rs56124762, rs1892231, rs16901748, rs9806914, rs1326860, rs12457255, rs2409750 |
| Model_317 | rs6478109, rs56124762, rs1892231, rs16901748, rs9806914, rs1326860, rs12457255, rs1541020 |
| Model_318 | rs6478109, rs56124762, rs1892231, rs16901748, rs9806914, rs1326860, rs12457255, rs4942248 |
| Model_319 | rs6478109, rs56124762, rs1892231, rs16901748, rs9806914, rs1326860, rs12457255, rs7759385 |
| Model_320 | rs6478109, rs56124762, rs1892231, rs16901748, rs9806914, rs1326860, rs2815844, rs10974900 |
| Model_321 | rs6478109, rs56124762, rs1892231, rs16901748, rs9806914, rs1326860, rs2815844, rs2409750 |
| Model_322 | rs6478109, rs56124762, rs1892231, rs16901748, rs9806914, rs1326860, rs2815844, rs1541020 |
| Model_323 | rs6478109, rs56124762, rs1892231, rs16901748, rs9806914, rs1326860, rs2815844, rs4942248 |
| Model_324 | rs6478109, rs56124762, rs1892231, rs16901748, rs9806914, rs1326860, rs2815844, rs7759385 |
| Model_325 | rs6478109, rs56124762, rs1892231, rs16901748, rs9806914, rs1326860, rs10974900, rs2409750 |
| Model_326 | rs6478109, rs56124762, rs1892231, rs16901748, rs9806914, rs1326860, rs10974900, rs1541020 |
| Model_327 | rs6478109, rs56124762, rs1892231, rs16901748, rs9806914, rs1326860, rs10974900, rs4942248 |
| Model_328 | rs6478109, rs56124762, rs1892231, rs16901748, rs9806914, rs1326860, rs10974900, rs7759385 |
| Model_329 | rs6478109, rs56124762, rs1892231, rs16901748, rs9806914, rs1326860, rs2409750, rs1541020 |
| Model_330 | rs6478109, rs56124762, rs1892231, rs16901748, rs9806914, rs1326860, rs2409750, rs4942248 |
| Model_331 | rs6478109, rs56124762, rs1892231, rs16901748, rs9806914, rs1326860, rs2409750, rs7759385 |
| Model_332 | rs6478109, rs56124762, rs1892231, rs16901748, rs9806914, rs1326860, rs1541020, rs4942248 |
| Model_333 | rs6478109, rs56124762, rs1892231, rs16901748, rs9806914, rs1326860, rs1541020, rs7759385 |
| Model_334 | rs6478109, rs56124762, rs1892231, rs16901748, rs9806914, rs1326860, rs4942248, rs7759385 |
| Model_335 | rs6478109, rs56124762, rs1892231, rs16901748, rs9806914, rs12457255, rs2815844, rs10974900 |
| Model_336 | rs6478109, rs56124762, rs1892231, rs16901748, rs9806914, rs12457255, rs2815844, rs2409750 |
| Model_337 | rs6478109, rs56124762, rs1892231, rs16901748, rs9806914, rs12457255, rs2815844, rs1541020 |
| Model_338 | rs6478109, rs56124762, rs1892231, rs16901748, rs9806914, rs12457255, rs2815844, rs4942248 |
| Model_339 | rs6478109, rs56124762, rs1892231, rs16901748, rs9806914, rs12457255, rs2815844, rs7759385 |
| Model_340 | rs6478109, rs56124762, rs1892231, rs16901748, rs9806914, rs12457255, rs10974900, rs2409750 |
| Model_341 | rs6478109, rs56124762, rs1892231, rs16901748, rs9806914, rs12457255, rs10974900, rs1541020 |
| Model_342 | rs6478109, rs56124762, rs1892231, rs16901748, rs9806914, rs12457255, rs10974900, rs4942248 |
| Model_343 | rs6478109, rs56124762, rs1892231, rs16901748, rs9806914, rs12457255, rs10974900, rs7759385 |
| Model_344 | rs6478109, rs56124762, rs1892231, rs16901748, rs9806914, rs12457255, rs2409750, rs1541020 |
| Model_345 | rs6478109, rs56124762, rs1892231, rs16901748, rs9806914, rs12457255, rs2409750, rs4942248 |
| Model_346 | rs6478109, rs56124762, rs1892231, rs16901748, rs9806914, rs12457255, rs2409750, rs7759385 |
| Model_347 | rs6478109, rs56124762, rs1892231, rs16901748, rs9806914, rs12457255, rs1541020, rs4942248 |
| Model_348 | rs6478109, rs56124762, rs1892231, rs16901748, rs9806914, rs12457255, rs1541020, rs7759385 |
| Model_349 | rs6478109, rs56124762, rs1892231, rs16901748, rs9806914, rs12457255, rs4942248, rs7759385 |
| Model_350 | rs6478109, rs56124762, rs1892231, rs16901748, rs9806914, rs2815844, rs10974900, rs2409750 |
| Model_351 | rs6478109, rs56124762, rs1892231, rs16901748, rs9806914, rs2815844, rs10974900, rs1541020 |
| Model_352 | rs6478109, rs56124762, rs1892231, rs16901748, rs9806914, rs2815844, rs10974900, rs4942248 |
| Model_353 | rs6478109, rs56124762, rs1892231, rs16901748, rs9806914, rs2815844, rs10974900, rs7759385 |
| Model_354 | rs6478109, rs56124762, rs1892231, rs16901748, rs9806914, rs2815844, rs2409750, rs1541020 |
| Model_355 | rs6478109, rs56124762, rs1892231, rs16901748, rs9806914, rs2815844, rs2409750, rs4942248 |
| Model_356 | rs6478109, rs56124762, rs1892231, rs16901748, rs9806914, rs2815844, rs2409750, rs7759385 |
| Model_357 | rs6478109, rs56124762, rs1892231, rs16901748, rs9806914, rs2815844, rs1541020, rs4942248 |
| Model_358 | rs6478109, rs56124762, rs1892231, rs16901748, rs9806914, rs2815844, rs1541020, rs7759385 |
| Model_359 | rs6478109, rs56124762, rs1892231, rs16901748, rs9806914, rs2815844, rs4942248, rs7759385 |
| Model_360 | rs6478109, rs56124762, rs1892231, rs16901748, rs9806914, rs10974900, rs2409750, rs1541020 |
| Model_361 | rs6478109, rs56124762, rs1892231, rs16901748, rs9806914, rs10974900, rs2409750, rs4942248 |
| Model_362 | rs6478109, rs56124762, rs1892231, rs16901748, rs9806914, rs10974900, rs2409750, rs7759385 |
| Model_363 | rs6478109, rs56124762, rs1892231, rs16901748, rs9806914, rs10974900, rs1541020, rs4942248 |
| Model_364 | rs6478109, rs56124762, rs1892231, rs16901748, rs9806914, rs10974900, rs1541020, rs7759385 |
| Model_365 | rs6478109, rs56124762, rs1892231, rs16901748, rs9806914, rs10974900, rs4942248, rs7759385 |
| Model_366 | rs6478109, rs56124762, rs1892231, rs16901748, rs9806914, rs2409750, rs1541020, rs4942248 |
| Model_367 | rs6478109, rs56124762, rs1892231, rs16901748, rs9806914, rs2409750, rs1541020, rs7759385 |
| Model_368 | rs6478109, rs56124762, rs1892231, rs16901748, rs9806914, rs2409750, rs4942248, rs7759385 |
| Model_369 | rs6478109, rs56124762, rs1892231, rs16901748, rs9806914, rs1541020, rs4942248, rs7759385 |
| Model_370 | rs6478109, rs56124762, rs1892231, rs16901748, rs2297437, rs1326860, rs12457255, rs2815844 |
| Model_371 | rs6478109, rs56124762, rs1892231, rs16901748, rs2297437, rs1326860, rs12457255, rs10974900 |
| Model_372 | rs6478109, rs56124762, rs1892231, rs16901748, rs2297437, rs1326860, rs12457255, rs2409750 |
| Model_373 | rs6478109, rs56124762, rs1892231, rs16901748, rs2297437, rs1326860, rs12457255, rs1541020 |
| Model_374 | rs6478109, rs56124762, rs1892231, rs16901748, rs2297437, rs1326860, rs12457255, rs4942248 |
| Model_375 | rs6478109, rs56124762, rs1892231, rs16901748, rs2297437, rs1326860, rs12457255, rs7759385 |
| Model_376 | rs6478109, rs56124762, rs1892231, rs16901748, rs2297437, rs1326860, rs2815844, rs10974900 |
| Model_377 | rs6478109, rs56124762, rs1892231, rs16901748, rs2297437, rs1326860, rs2815844, rs2409750 |
| Model_378 | rs6478109, rs56124762, rs1892231, rs16901748, rs2297437, rs1326860, rs2815844, rs1541020 |
| Model_379 | rs6478109, rs56124762, rs1892231, rs16901748, rs2297437, rs1326860, rs2815844, rs4942248 |
| Model_380 | rs6478109, rs56124762, rs1892231, rs16901748, rs2297437, rs1326860, rs2815844, rs7759385 |
| Model_381 | rs6478109, rs56124762, rs1892231, rs16901748, rs2297437, rs1326860, rs10974900, rs2409750 |
| Model_382 | rs6478109, rs56124762, rs1892231, rs16901748, rs2297437, rs1326860, rs10974900, rs1541020 |
| Model_383 | rs6478109, rs56124762, rs1892231, rs16901748, rs2297437, rs1326860, rs10974900, rs4942248 |
| Model_384 | rs6478109, rs56124762, rs1892231, rs16901748, rs2297437, rs1326860, rs10974900, rs7759385 |
| Model_385 | rs6478109, rs56124762, rs1892231, rs16901748, rs2297437, rs1326860, rs2409750, rs1541020 |
| Model_386 | rs6478109, rs56124762, rs1892231, rs16901748, rs2297437, rs1326860, rs2409750, rs4942248 |
| Model_387 | rs6478109, rs56124762, rs1892231, rs16901748, rs2297437, rs1326860, rs2409750, rs7759385 |
| Model_388 | rs6478109, rs56124762, rs1892231, rs16901748, rs2297437, rs1326860, rs1541020, rs4942248 |
| Model_389 | rs6478109, rs56124762, rs1892231, rs16901748, rs2297437, rs1326860, rs1541020, rs7759385 |
| Model_390 | rs6478109, rs56124762, rs1892231, rs16901748, rs2297437, rs1326860, rs4942248, rs7759385 |
| Model_391 | rs6478109, rs56124762, rs1892231, rs16901748, rs2297437, rs12457255, rs2815844, rs10974900 |
| Model_392 | rs6478109, rs56124762, rs1892231, rs16901748, rs2297437, rs12457255, rs2815844, rs2409750 |
| Model_393 | rs6478109, rs56124762, rs1892231, rs16901748, rs2297437, rs12457255, rs2815844, rs1541020 |
| Model_394 | rs6478109, rs56124762, rs1892231, rs16901748, rs2297437, rs12457255, rs2815844, rs4942248 |
| Model_395 | rs6478109, rs56124762, rs1892231, rs16901748, rs2297437, rs12457255, rs2815844, rs7759385 |
| Model_396 | rs6478109, rs56124762, rs1892231, rs16901748, rs2297437, rs12457255, rs10974900, rs2409750 |
| Model_397 | rs6478109, rs56124762, rs1892231, rs16901748, rs2297437, rs12457255, rs10974900, rs1541020 |
| Model_398 | rs6478109, rs56124762, rs1892231, rs16901748, rs2297437, rs12457255, rs10974900, rs4942248 |
| Model_399 | rs6478109, rs56124762, rs1892231, rs16901748, rs2297437, rs12457255, rs10974900, rs7759385 |
| Model_400 | rs6478109, rs56124762, rs1892231, rs16901748, rs2297437, rs12457255, rs2409750, rs1541020 |
| Model_401 | rs6478109, rs56124762, rs1892231, rs16901748, rs2297437, rs12457255, rs2409750, rs4942248 |
| Model_402 | rs6478109, rs56124762, rs1892231, rs16901748, rs2297437, rs12457255, rs2409750, rs7759385 |
| Model_403 | rs6478109, rs56124762, rs1892231, rs16901748, rs2297437, rs12457255, rs1541020, rs4942248 |
| Model_404 | rs6478109, rs56124762, rs1892231, rs16901748, rs2297437, rs12457255, rs1541020, rs7759385 |
| Model_405 | rs6478109, rs56124762, rs1892231, rs16901748, rs2297437, rs12457255, rs4942248, rs7759385 |
| Model_406 | rs6478109, rs56124762, rs1892231, rs16901748, rs2297437, rs2815844, rs10974900, rs2409750 |
| Model_407 | rs6478109, rs56124762, rs1892231, rs16901748, rs2297437, rs2815844, rs10974900, rs1541020 |
| Model_408 | rs6478109, rs56124762, rs1892231, rs16901748, rs2297437, rs2815844, rs10974900, rs4942248 |
| Model_409 | rs6478109, rs56124762, rs1892231, rs16901748, rs2297437, rs2815844, rs10974900, rs7759385 |
| Model_410 | rs6478109, rs56124762, rs1892231, rs16901748, rs2297437, rs2815844, rs2409750, rs1541020 |
| Model_411 | rs6478109, rs56124762, rs1892231, rs16901748, rs2297437, rs2815844, rs2409750, rs4942248 |
| Model_412 | rs6478109, rs56124762, rs1892231, rs16901748, rs2297437, rs2815844, rs2409750, rs7759385 |
| Model_413 | rs6478109, rs56124762, rs1892231, rs16901748, rs2297437, rs2815844, rs1541020, rs4942248 |
| Model_414 | rs6478109, rs56124762, rs1892231, rs16901748, rs2297437, rs2815844, rs1541020, rs7759385 |
| Model_415 | rs6478109, rs56124762, rs1892231, rs16901748, rs2297437, rs2815844, rs4942248, rs7759385 |
| Model_416 | rs6478109, rs56124762, rs1892231, rs16901748, rs2297437, rs10974900, rs2409750, rs1541020 |
| Model_417 | rs6478109, rs56124762, rs1892231, rs16901748, rs2297437, rs10974900, rs2409750, rs4942248 |
| Model_418 | rs6478109, rs56124762, rs1892231, rs16901748, rs2297437, rs10974900, rs2409750, rs7759385 |
| Model_419 | rs6478109, rs56124762, rs1892231, rs16901748, rs2297437, rs10974900, rs1541020, rs4942248 |
| Model_420 | rs6478109, rs56124762, rs1892231, rs16901748, rs2297437, rs10974900, rs1541020, rs7759385 |
| Model_421 | rs6478109, rs56124762, rs1892231, rs16901748, rs2297437, rs10974900, rs4942248, rs7759385 |
| Model_422 | rs6478109, rs56124762, rs1892231, rs16901748, rs2297437, rs2409750, rs1541020, rs4942248 |
| Model_423 | rs6478109, rs56124762, rs1892231, rs16901748, rs2297437, rs2409750, rs1541020, rs7759385 |
| Model_424 | rs6478109, rs56124762, rs1892231, rs16901748, rs2297437, rs2409750, rs4942248, rs7759385 |
| Model_425 | rs6478109, rs56124762, rs1892231, rs16901748, rs2297437, rs1541020, rs4942248, rs7759385 |
| Model_426 | rs6478109, rs56124762, rs1892231, rs16901748, rs1326860, rs12457255, rs2815844, rs10974900 |
| Model_427 | rs6478109, rs56124762, rs1892231, rs16901748, rs1326860, rs12457255, rs2815844, rs2409750 |
| Model_428 | rs6478109, rs56124762, rs1892231, rs16901748, rs1326860, rs12457255, rs2815844, rs1541020 |
| Model_429 | rs6478109, rs56124762, rs1892231, rs16901748, rs1326860, rs12457255, rs2815844, rs4942248 |
| Model_430 | rs6478109, rs56124762, rs1892231, rs16901748, rs1326860, rs12457255, rs2815844, rs7759385 |
| Model_431 | rs6478109, rs56124762, rs1892231, rs16901748, rs1326860, rs12457255, rs10974900, rs2409750 |
| Model_432 | rs6478109, rs56124762, rs1892231, rs16901748, rs1326860, rs12457255, rs10974900, rs1541020 |
| Model_433 | rs6478109, rs56124762, rs1892231, rs16901748, rs1326860, rs12457255, rs10974900, rs4942248 |
| Model_434 | rs6478109, rs56124762, rs1892231, rs16901748, rs1326860, rs12457255, rs10974900, rs7759385 |
| Model_435 | rs6478109, rs56124762, rs1892231, rs16901748, rs1326860, rs12457255, rs2409750, rs1541020 |
| Model_436 | rs6478109, rs56124762, rs1892231, rs16901748, rs1326860, rs12457255, rs2409750, rs4942248 |
| Model_437 | rs6478109, rs56124762, rs1892231, rs16901748, rs1326860, rs12457255, rs2409750, rs7759385 |
| Model_438 | rs6478109, rs56124762, rs1892231, rs16901748, rs1326860, rs12457255, rs1541020, rs4942248 |
| Model_439 | rs6478109, rs56124762, rs1892231, rs16901748, rs1326860, rs12457255, rs1541020, rs7759385 |
| Model_440 | rs6478109, rs56124762, rs1892231, rs16901748, rs1326860, rs12457255, rs4942248, rs7759385 |
| Model_441 | rs6478109, rs56124762, rs1892231, rs16901748, rs1326860, rs2815844, rs10974900, rs2409750 |
| Model_442 | rs6478109, rs56124762, rs1892231, rs16901748, rs1326860, rs2815844, rs10974900, rs1541020 |
| Model_443 | rs6478109, rs56124762, rs1892231, rs16901748, rs1326860, rs2815844, rs10974900, rs4942248 |
| Model_444 | rs6478109, rs56124762, rs1892231, rs16901748, rs1326860, rs2815844, rs10974900, rs7759385 |
| Model_445 | rs6478109, rs56124762, rs1892231, rs16901748, rs1326860, rs2815844, rs2409750, rs1541020 |
| Model_446 | rs6478109, rs56124762, rs1892231, rs16901748, rs1326860, rs2815844, rs2409750, rs4942248 |
| Model_447 | rs6478109, rs56124762, rs1892231, rs16901748, rs1326860, rs2815844, rs2409750, rs7759385 |
| Model_448 | rs6478109, rs56124762, rs1892231, rs16901748, rs1326860, rs2815844, rs1541020, rs4942248 |
| Model_449 | rs6478109, rs56124762, rs1892231, rs16901748, rs1326860, rs2815844, rs1541020, rs7759385 |
| Model_450 | rs6478109, rs56124762, rs1892231, rs16901748, rs1326860, rs2815844, rs4942248, rs7759385 |
| Model_451 | rs6478109, rs56124762, rs1892231, rs16901748, rs1326860, rs10974900, rs2409750, rs1541020 |
| Model_452 | rs6478109, rs56124762, rs1892231, rs16901748, rs1326860, rs10974900, rs2409750, rs4942248 |
| Model_453 | rs6478109, rs56124762, rs1892231, rs16901748, rs1326860, rs10974900, rs2409750, rs7759385 |
| Model_454 | rs6478109, rs56124762, rs1892231, rs16901748, rs1326860, rs10974900, rs1541020, rs4942248 |
| Model_455 | rs6478109, rs56124762, rs1892231, rs16901748, rs1326860, rs10974900, rs1541020, rs7759385 |
| Model_456 | rs6478109, rs56124762, rs1892231, rs16901748, rs1326860, rs10974900, rs4942248, rs7759385 |
| Model_457 | rs6478109, rs56124762, rs1892231, rs16901748, rs1326860, rs2409750, rs1541020, rs4942248 |
| Model_458 | rs6478109, rs56124762, rs1892231, rs16901748, rs1326860, rs2409750, rs1541020, rs7759385 |
| Model_459 | rs6478109, rs56124762, rs1892231, rs16901748, rs1326860, rs2409750, rs4942248, rs7759385 |
| Model_460 | rs6478109, rs56124762, rs1892231, rs16901748, rs1326860, rs1541020, rs4942248, rs7759385 |
| Model_461 | rs6478109, rs56124762, rs1892231, rs16901748, rs12457255, rs2815844, rs10974900, rs2409750 |
| Model_462 | rs6478109, rs56124762, rs1892231, rs16901748, rs12457255, rs2815844, rs10974900, rs1541020 |
| Model_463 | rs6478109, rs56124762, rs1892231, rs16901748, rs12457255, rs2815844, rs10974900, rs4942248 |
| Model_464 | rs6478109, rs56124762, rs1892231, rs16901748, rs12457255, rs2815844, rs10974900, rs7759385 |
| Model_465 | rs6478109, rs56124762, rs1892231, rs16901748, rs12457255, rs2815844, rs2409750, rs1541020 |
| Model_466 | rs6478109, rs56124762, rs1892231, rs16901748, rs12457255, rs2815844, rs2409750, rs4942248 |
| Model_467 | rs6478109, rs56124762, rs1892231, rs16901748, rs12457255, rs2815844, rs2409750, rs7759385 |
| Model_468 | rs6478109, rs56124762, rs1892231, rs16901748, rs12457255, rs2815844, rs1541020, rs4942248 |
| Model_469 | rs6478109, rs56124762, rs1892231, rs16901748, rs12457255, rs2815844, rs1541020, rs7759385 |
| Model_470 | rs6478109, rs56124762, rs1892231, rs16901748, rs12457255, rs2815844, rs4942248, rs7759385 |
| Model_471 | rs6478109, rs56124762, rs1892231, rs16901748, rs12457255, rs10974900, rs2409750, rs1541020 |
| Model_472 | rs6478109, rs56124762, rs1892231, rs16901748, rs12457255, rs10974900, rs2409750, rs4942248 |
| Model_473 | rs6478109, rs56124762, rs1892231, rs16901748, rs12457255, rs10974900, rs2409750, rs7759385 |
| Model_474 | rs6478109, rs56124762, rs1892231, rs16901748, rs12457255, rs10974900, rs1541020, rs4942248 |
| Model_475 | rs6478109, rs56124762, rs1892231, rs16901748, rs12457255, rs10974900, rs1541020, rs7759385 |
| Model_476 | rs6478109, rs56124762, rs1892231, rs16901748, rs12457255, rs10974900, rs4942248, rs7759385 |
| Model_477 | rs6478109, rs56124762, rs1892231, rs16901748, rs12457255, rs2409750, rs1541020, rs4942248 |
| Model_478 | rs6478109, rs56124762, rs1892231, rs16901748, rs12457255, rs2409750, rs1541020, rs7759385 |
| Model_479 | rs6478109, rs56124762, rs1892231, rs16901748, rs12457255, rs2409750, rs4942248, rs7759385 |
| Model_480 | rs6478109, rs56124762, rs1892231, rs16901748, rs12457255, rs1541020, rs4942248, rs7759385 |
| Model_481 | rs6478109, rs56124762, rs1892231, rs16901748, rs2815844, rs10974900, rs2409750, rs1541020 |
| Model_482 | rs6478109, rs56124762, rs1892231, rs16901748, rs2815844, rs10974900, rs2409750, rs4942248 |
| Model_483 | rs6478109, rs56124762, rs1892231, rs16901748, rs2815844, rs10974900, rs2409750, rs7759385 |
| Model_484 | rs6478109, rs56124762, rs1892231, rs16901748, rs2815844, rs10974900, rs1541020, rs4942248 |
| Model_485 | rs6478109, rs56124762, rs1892231, rs16901748, rs2815844, rs10974900, rs1541020, rs7759385 |
| Model_486 | rs6478109, rs56124762, rs1892231, rs16901748, rs2815844, rs10974900, rs4942248, rs7759385 |
| Model_487 | rs6478109, rs56124762, rs1892231, rs16901748, rs2815844, rs2409750, rs1541020, rs4942248 |
| Model_488 | rs6478109, rs56124762, rs1892231, rs16901748, rs2815844, rs2409750, rs1541020, rs7759385 |
| Model_489 | rs6478109, rs56124762, rs1892231, rs16901748, rs2815844, rs2409750, rs4942248, rs7759385 |
| Model_490 | rs6478109, rs56124762, rs1892231, rs16901748, rs2815844, rs1541020, rs4942248, rs7759385 |
| Model_491 | rs6478109, rs56124762, rs1892231, rs16901748, rs10974900, rs2409750, rs1541020, rs4942248 |
| Model_492 | rs6478109, rs56124762, rs1892231, rs16901748, rs10974900, rs2409750, rs1541020, rs7759385 |
| Model_493 | rs6478109, rs56124762, rs1892231, rs16901748, rs10974900, rs2409750, rs4942248, rs7759385 |
| Model_494 | rs6478109, rs56124762, rs1892231, rs16901748, rs10974900, rs1541020, rs4942248, rs7759385 |
| Model_495 | rs6478109, rs56124762, rs1892231, rs16901748, rs2409750, rs1541020, rs4942248, rs7759385 |

### 7.20 Example 20: Optimization of SNP Model for TL1A Companion Diagnostics Based on Single Cell Deconvolution

As further described above, IBD, including CD or UC, can have heterogeneous disease pathology. The disclosure provides that IBD can be caused by various pathological mechanisms and driven by various inflammatory cell types. Thus the disclosure recognizes that certain inflammatory cell types are associated with TL1A-driven IBD (e.g. UC and/or CD), and the up- or down-regulation of these particular TL1A-associated inflammatory cell types can predict response or remission upon anti-TL1A therapies. Accordingly, optimization of SNP models for TL1A companion diagnostics was performed by examining the association of the SNP models, the up- or down-regulation of certain TL1A-associated inflammatory cell types, and the response or remission of IBD patients after TL1A treatment.

To determine the up- or down-regulation of TL1A-associated inflammatory cell types, single cell analyses of the IBD patient samples were performed. To perform single cell analyses, single cell gene expression profiles were first generated and then used to deconvolute the bulk RNA-sequence-based gene expression data of the IBD patient samples. Once the bulk RNA sequence data were deconvoluted into single cell level, studies were performed to determine the association between the SNP models that have been determined to associate with TL1A expression in immune cells (as determined in the previous examples) and the up- or down-regulation of certain inflammatory cell types. Such association was then used to build and optimize SNP models for predicting IBD patients' response or remission to anti-TL1A therapeutic agents.

The SNP models that both associated with TL1A expression in immune cells and with certain inflammatory cell types were selected as the candidate models to further test for prediction of response or remission of IBD patients to anti-TL1A therapeutic agents.

### 7.20.1 Subpart 1. Generation of Single Cell Data Profile as Reference for Deconvolution

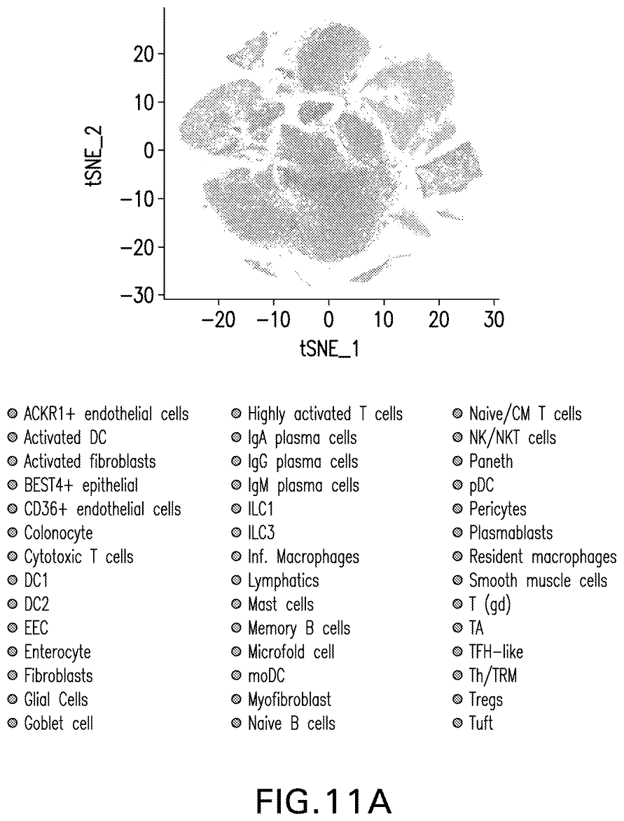

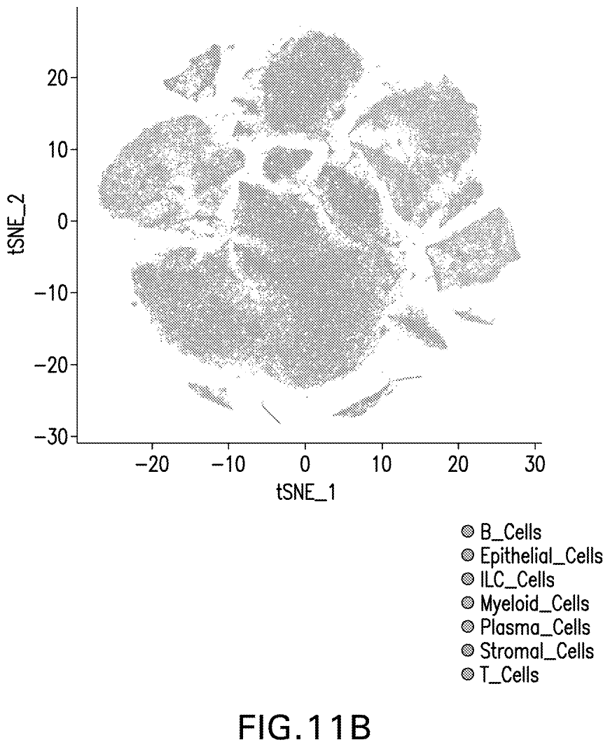

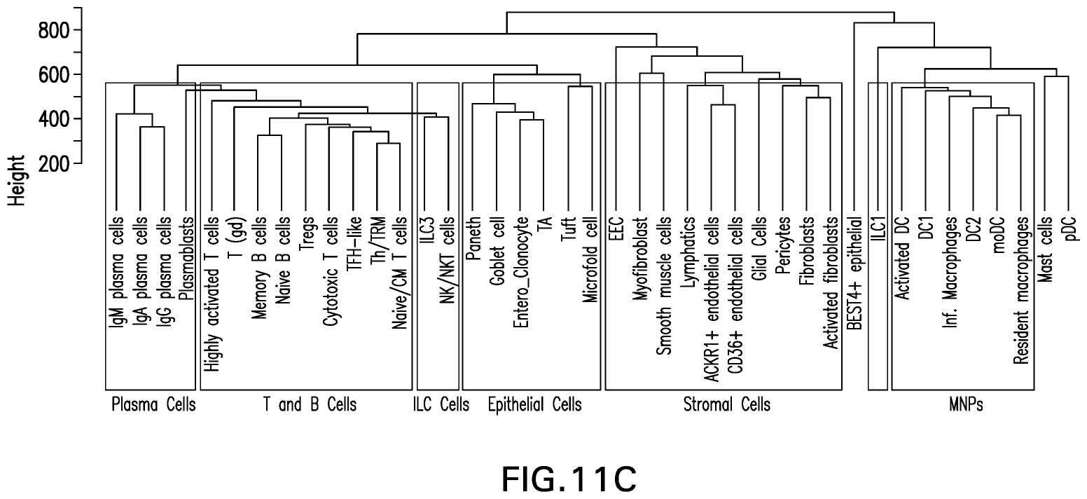

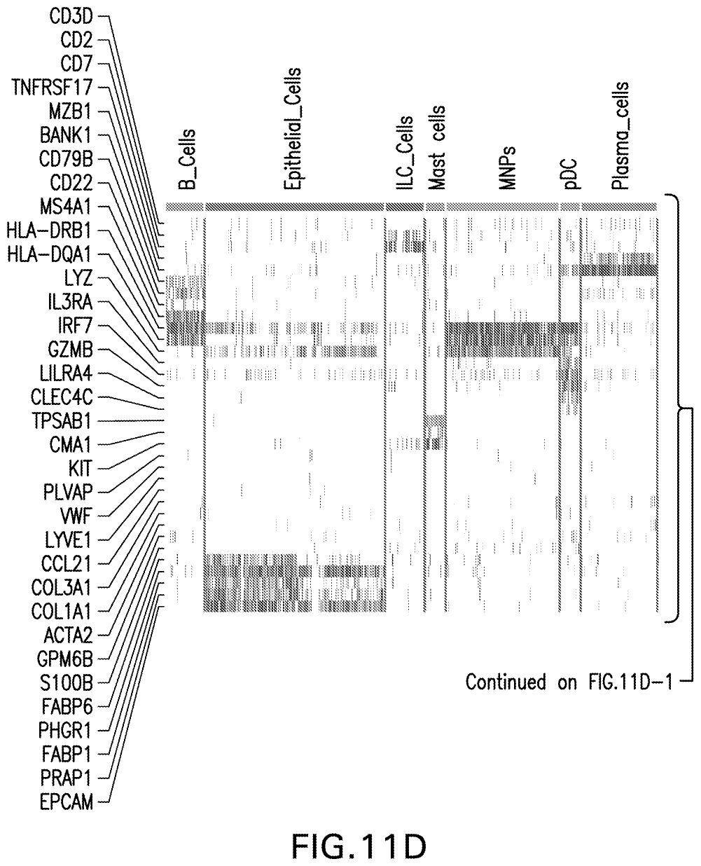

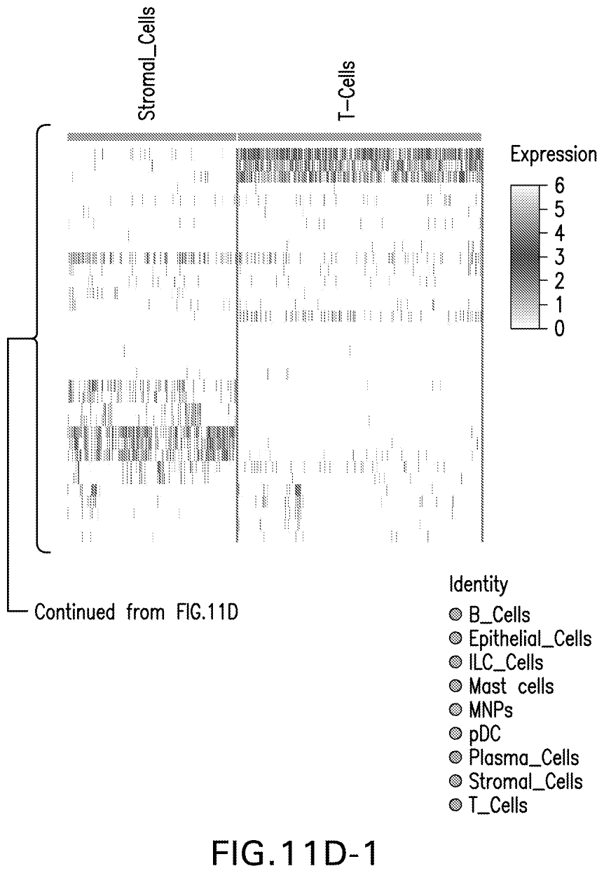

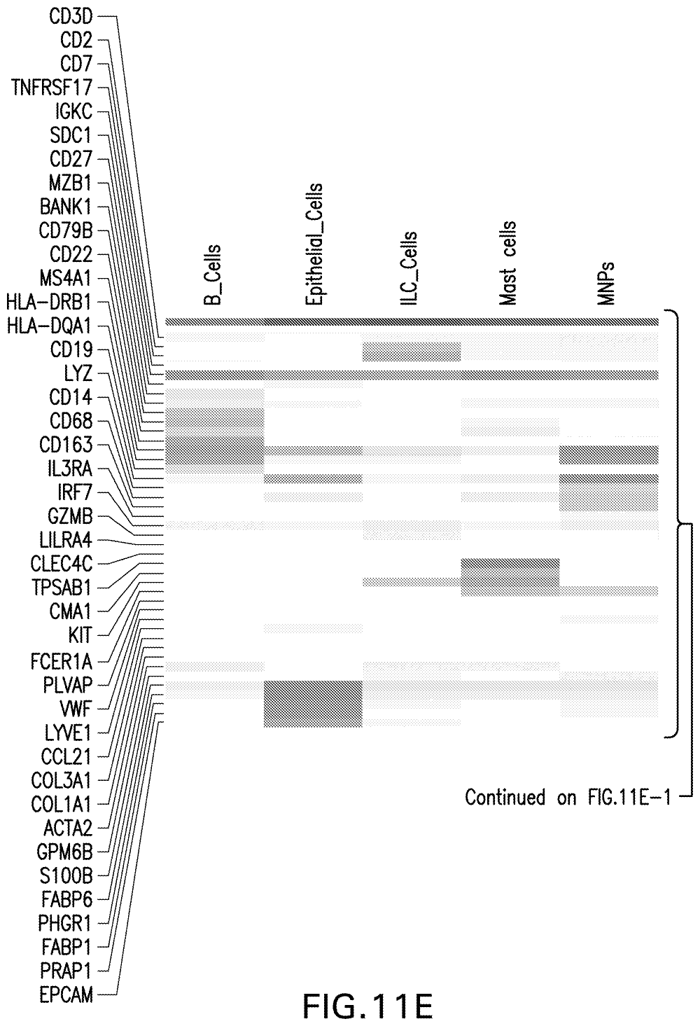

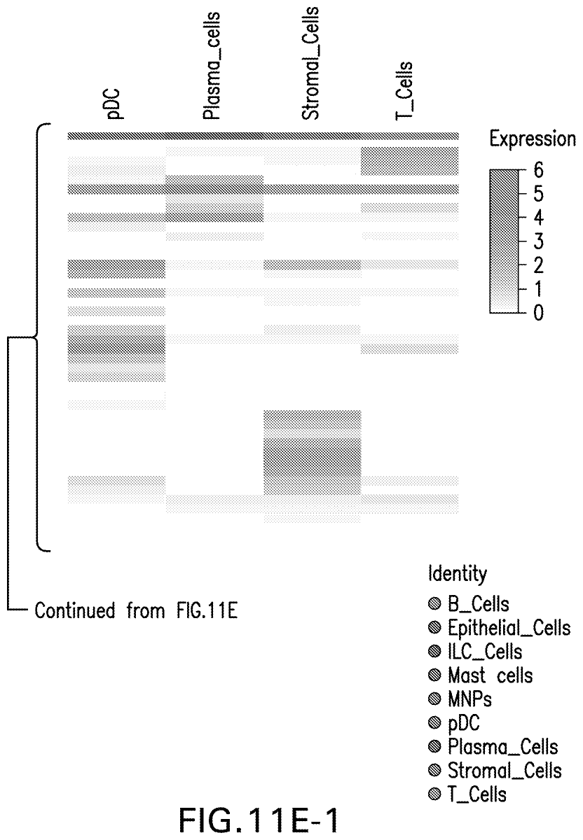

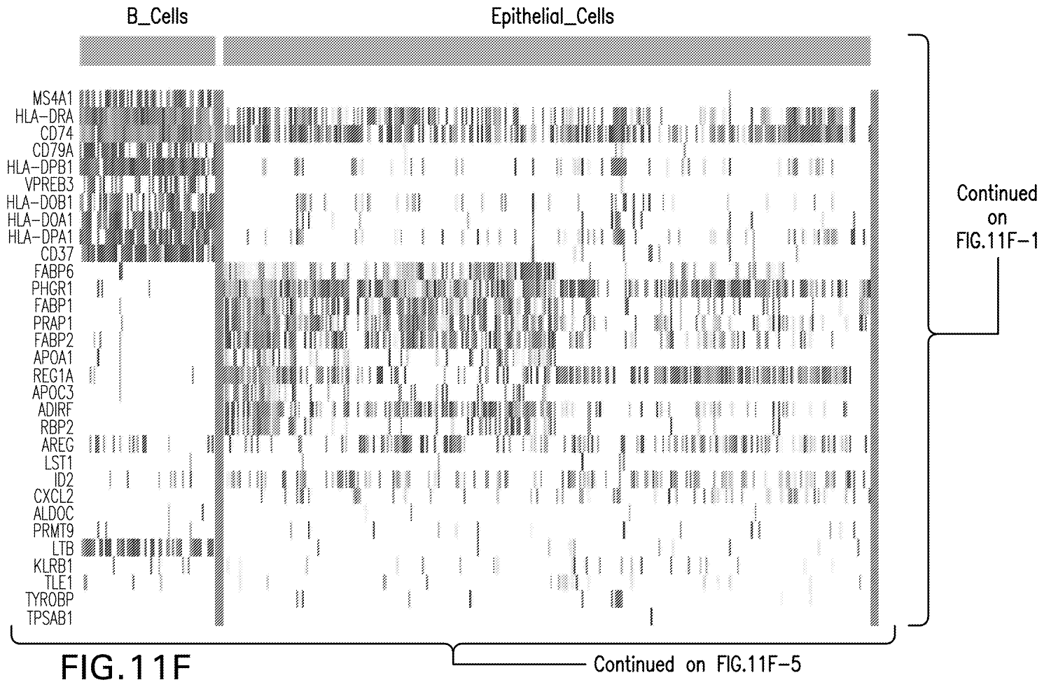

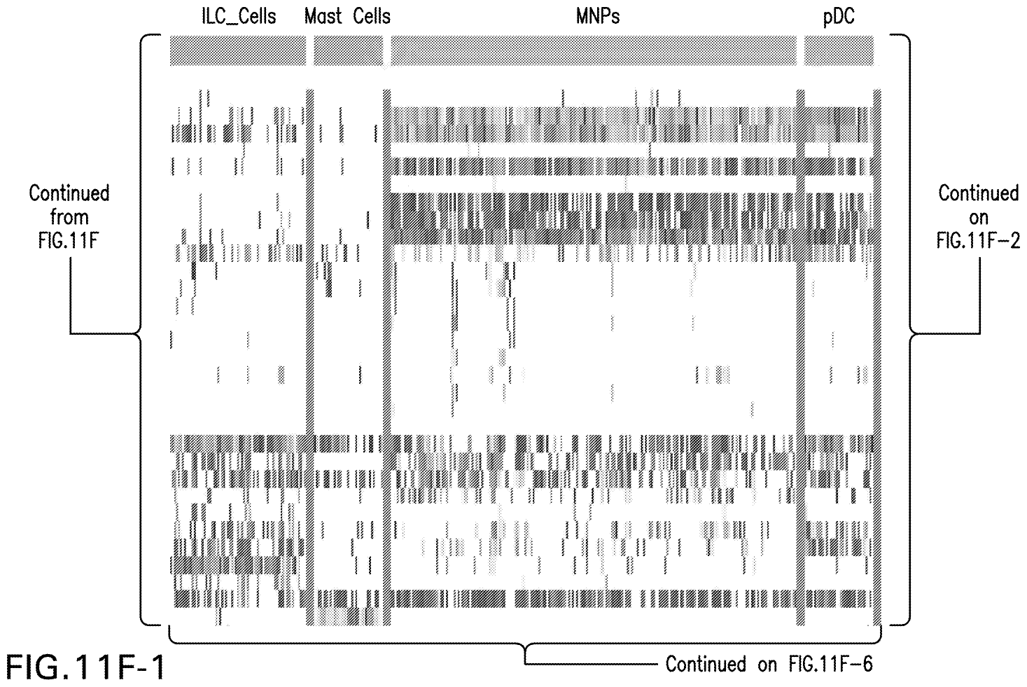

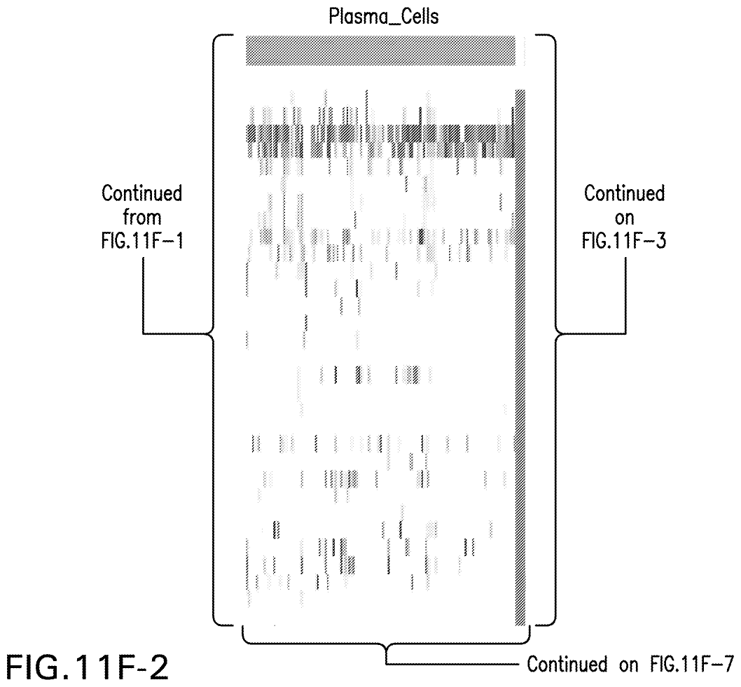

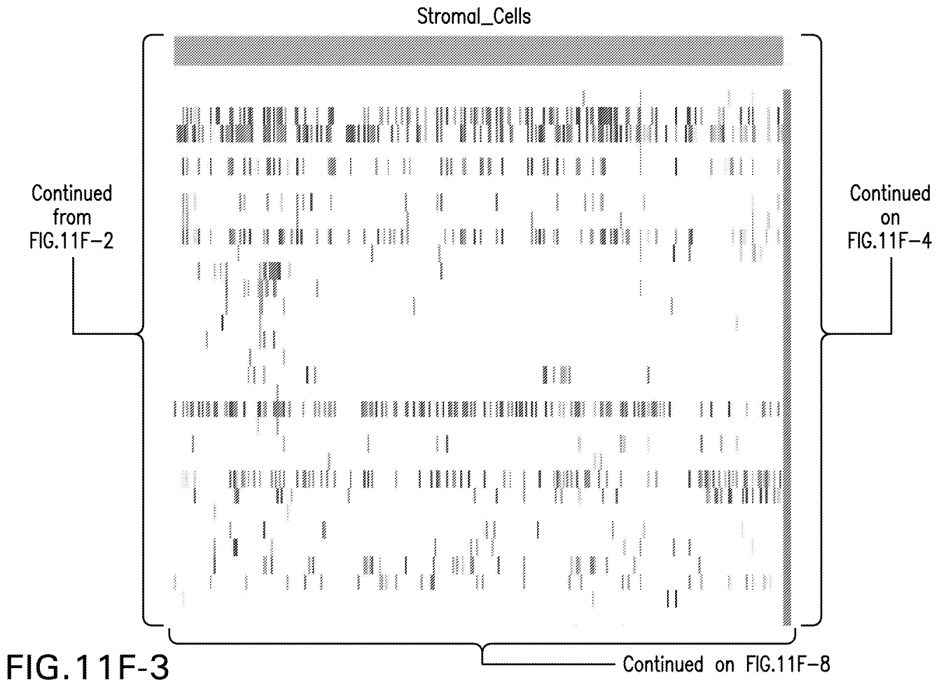

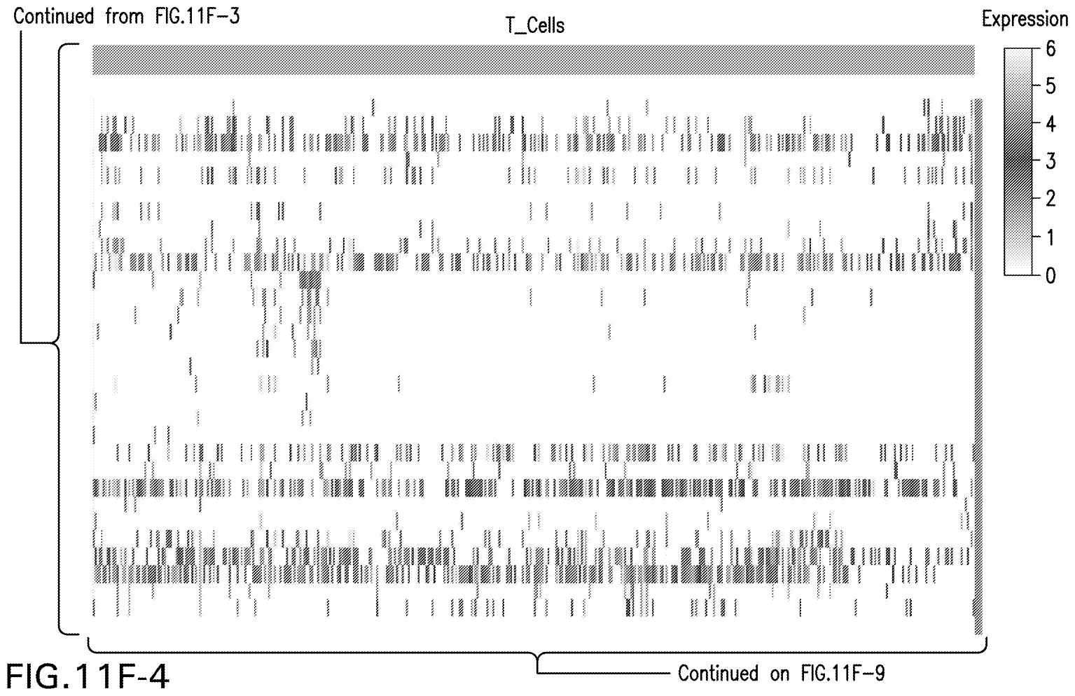

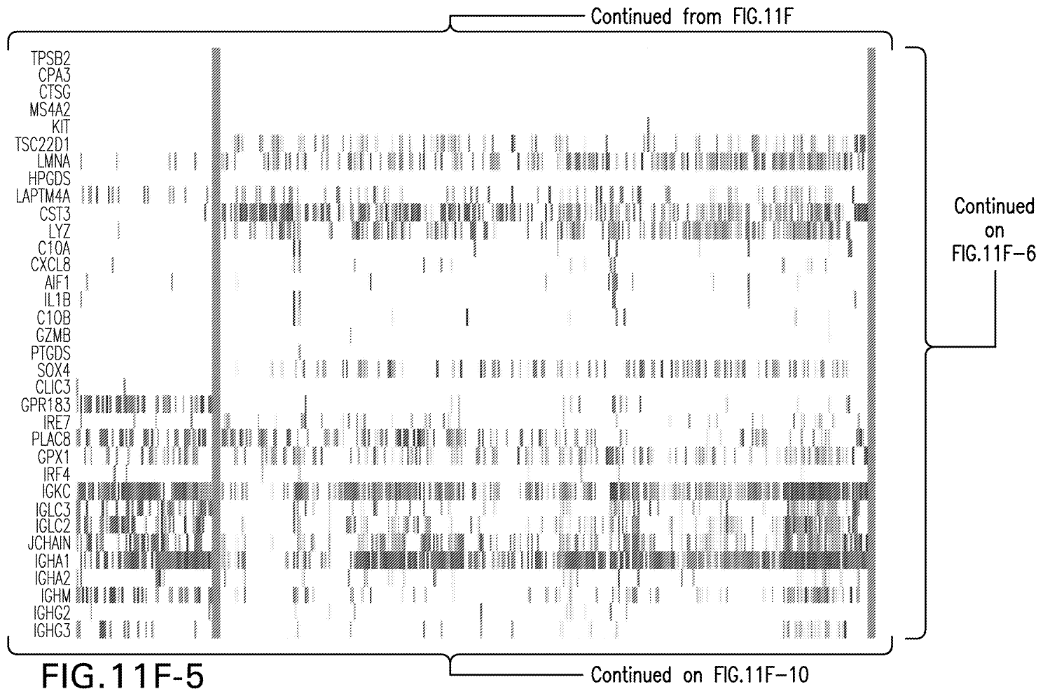

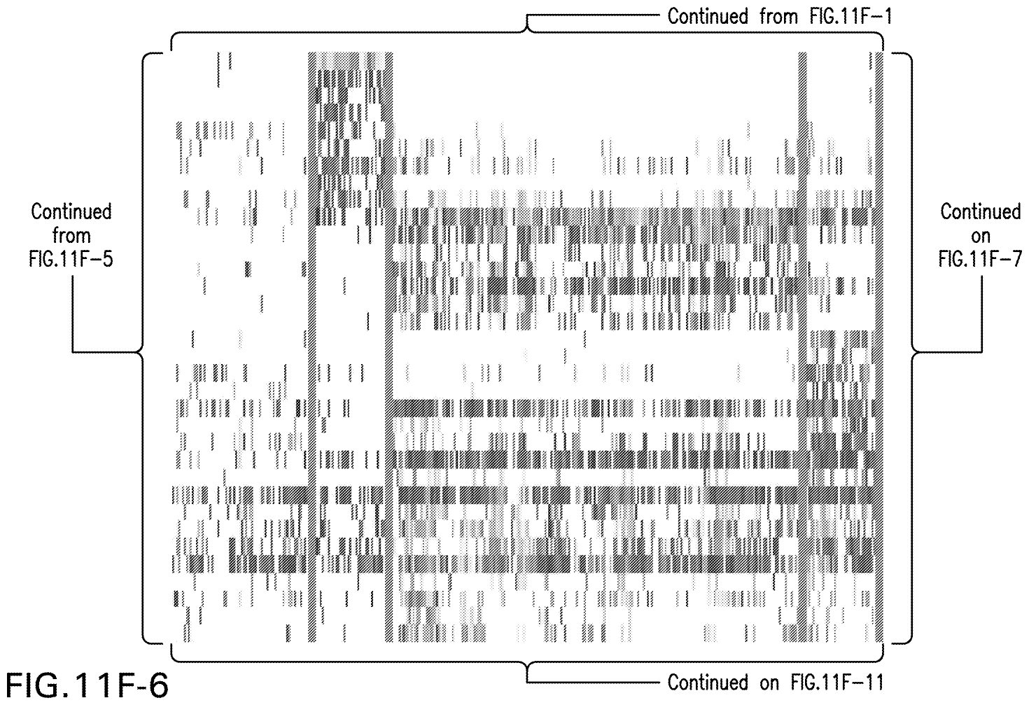

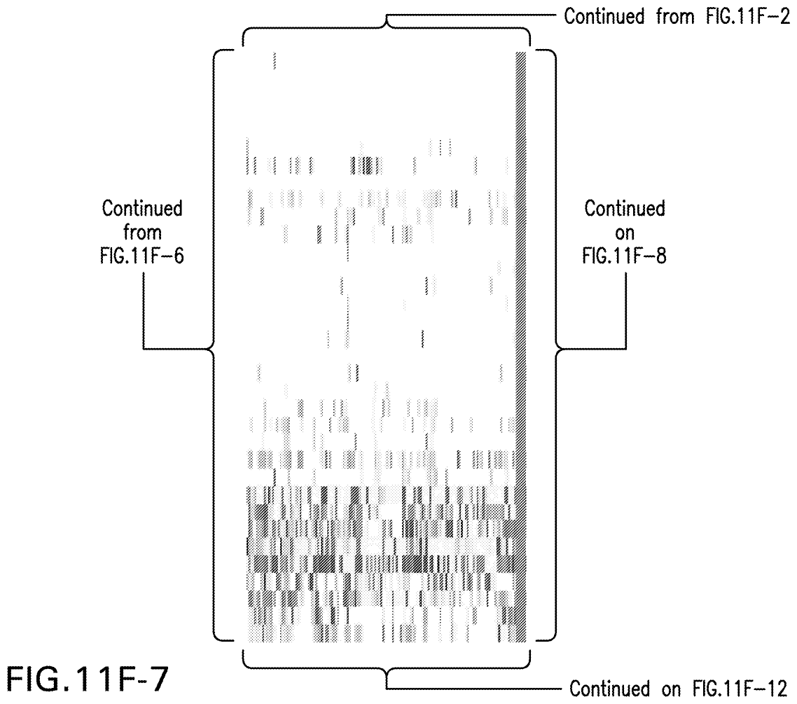

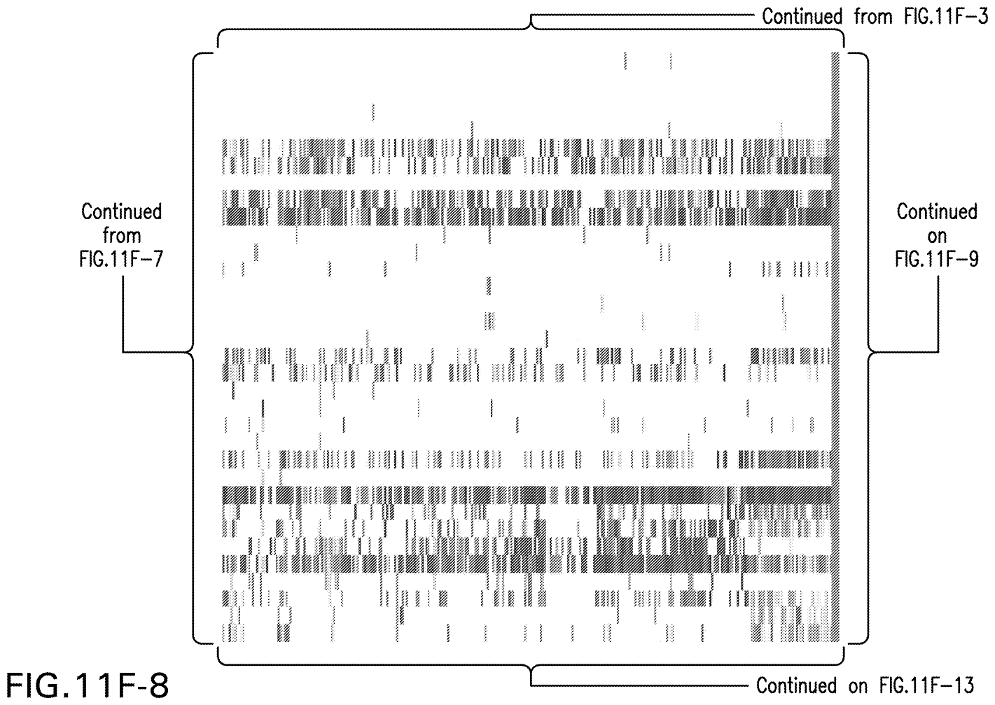

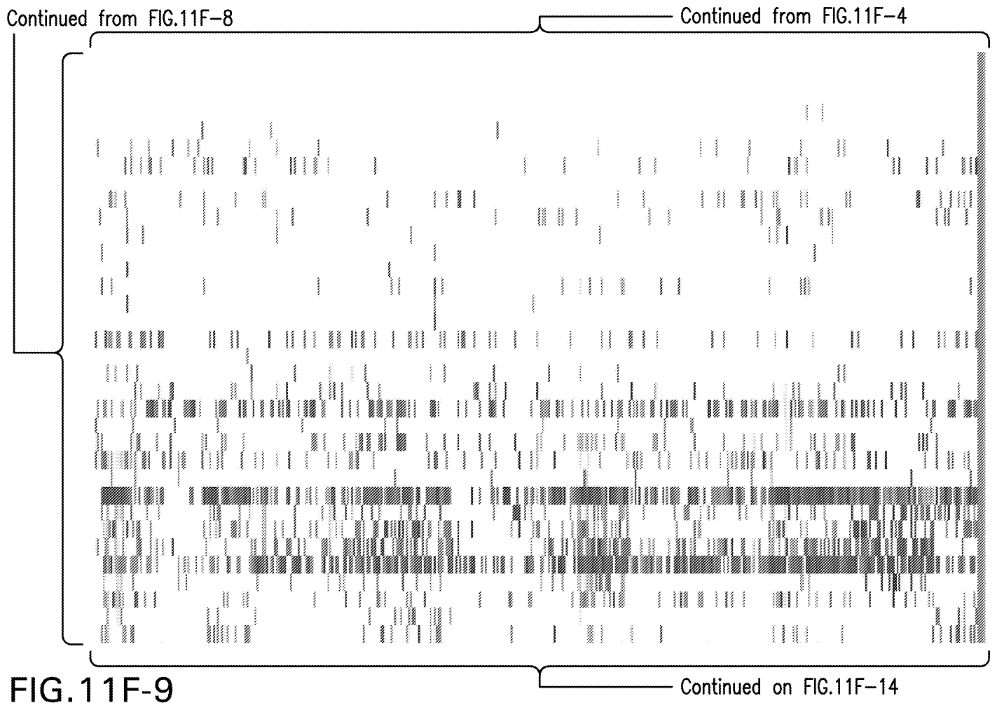

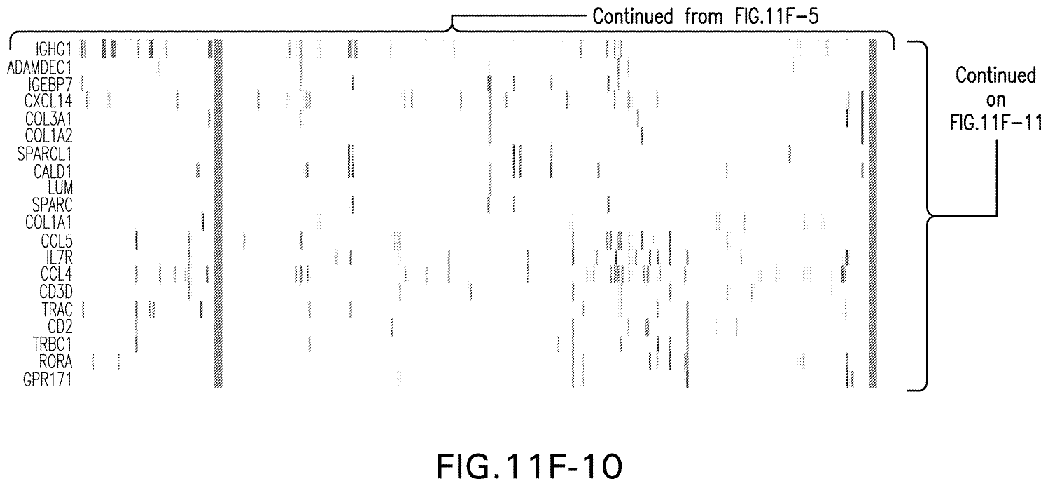

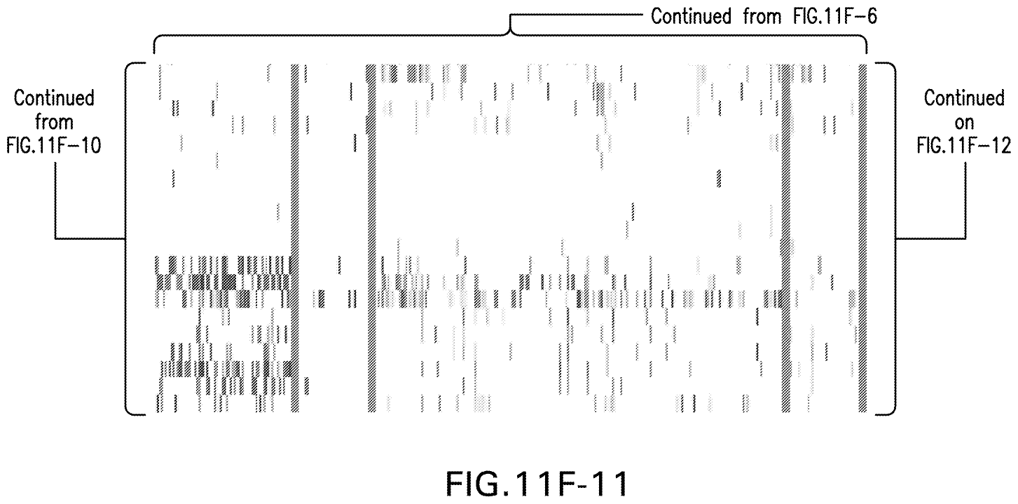

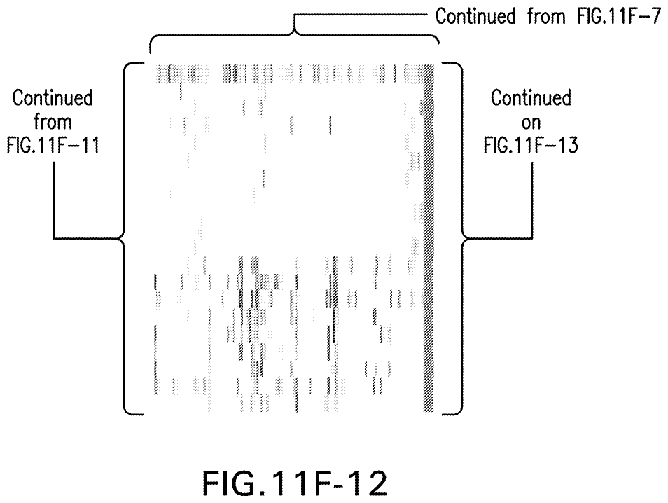

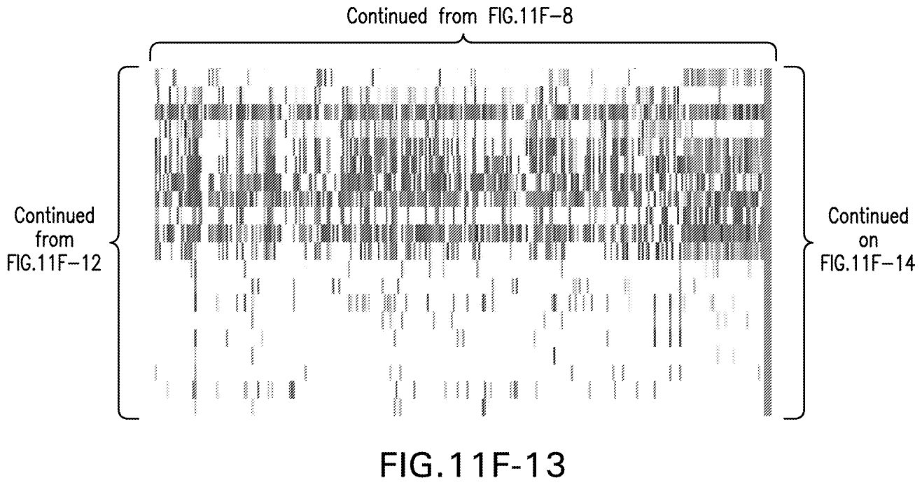

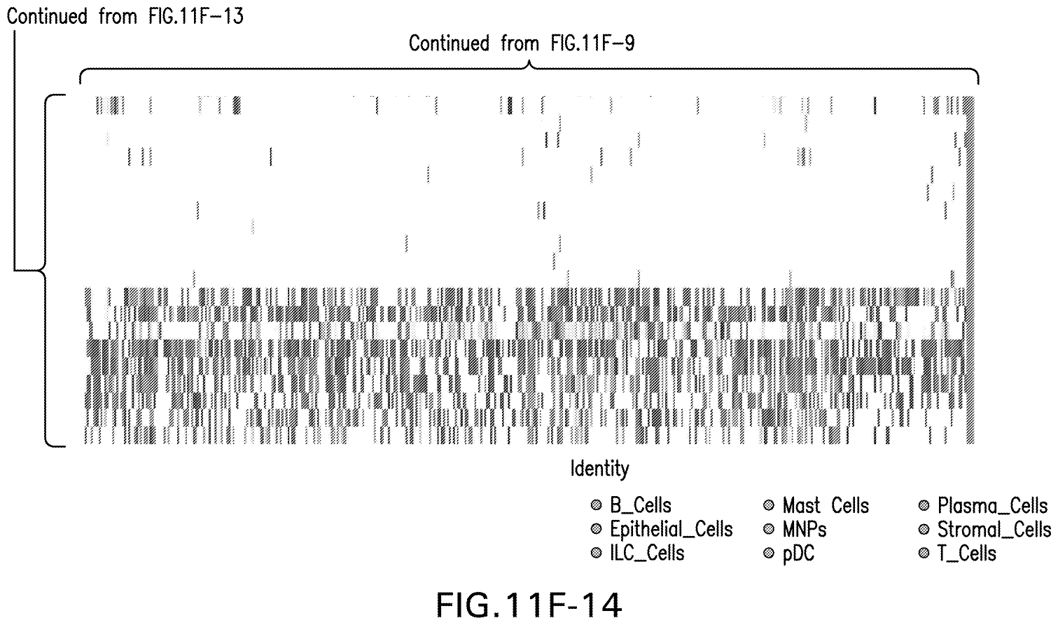

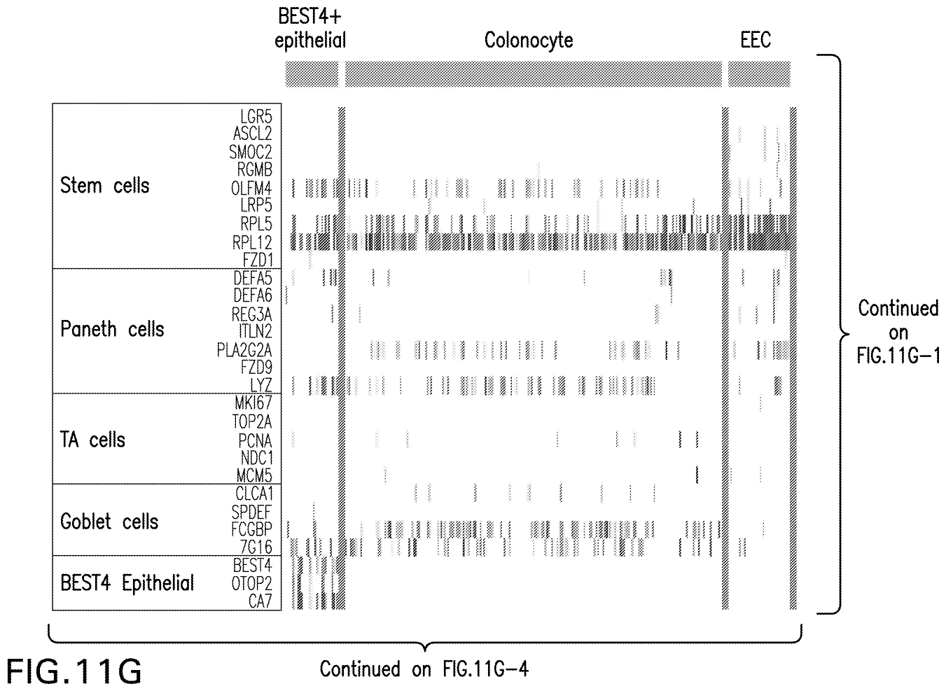

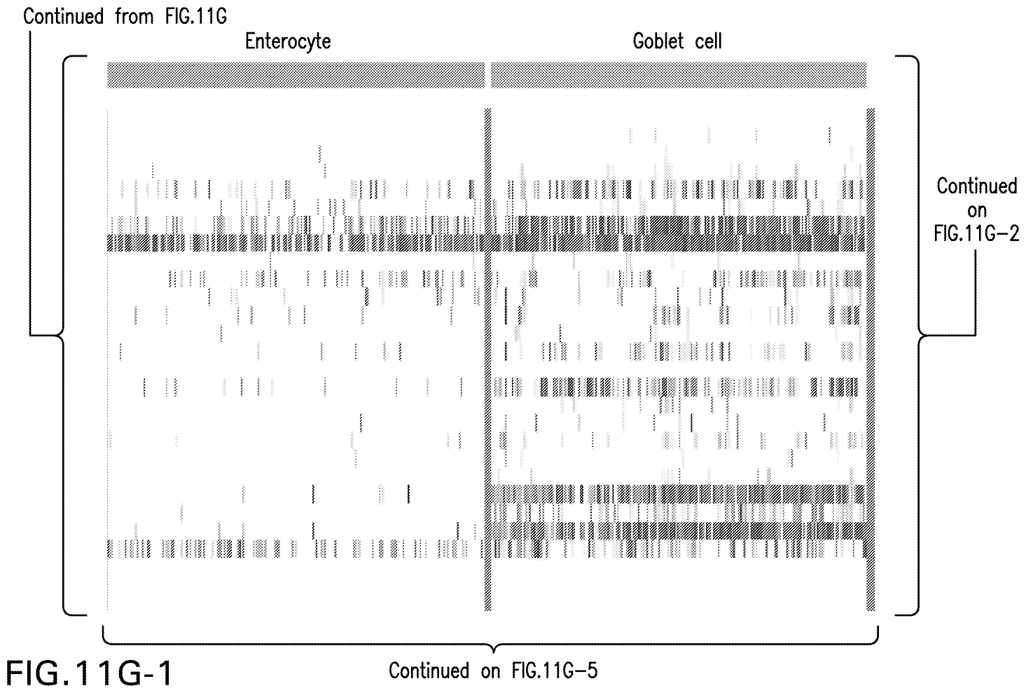

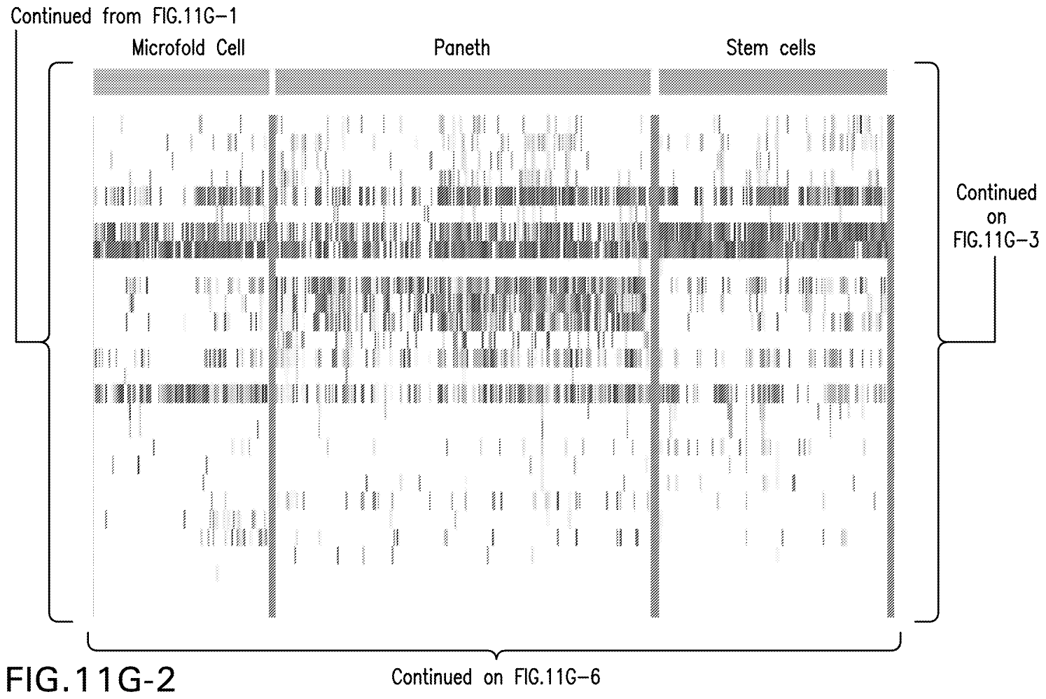

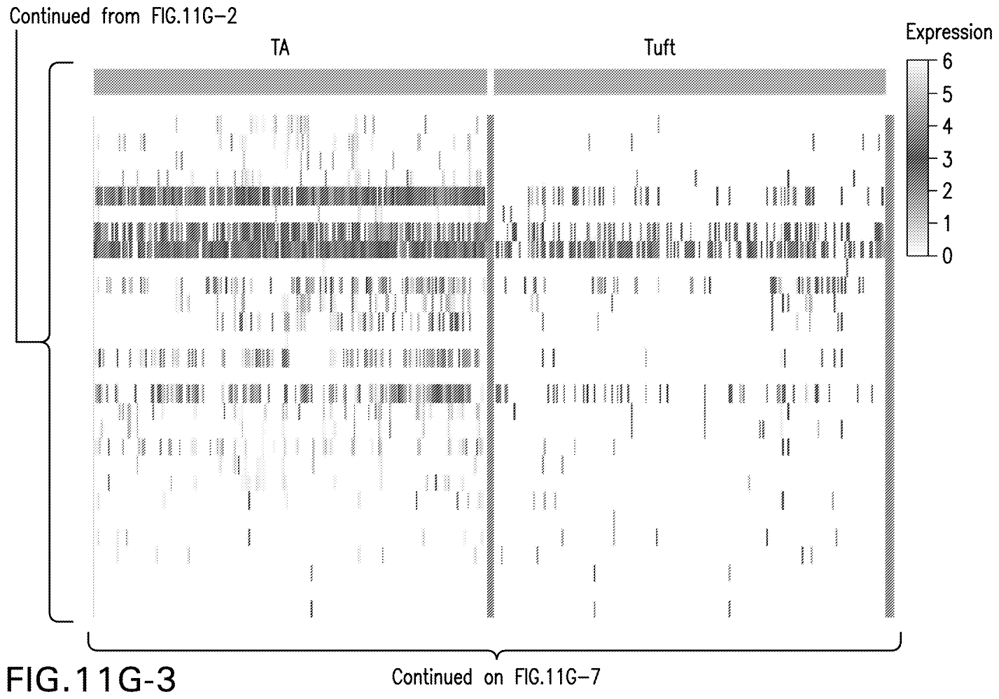

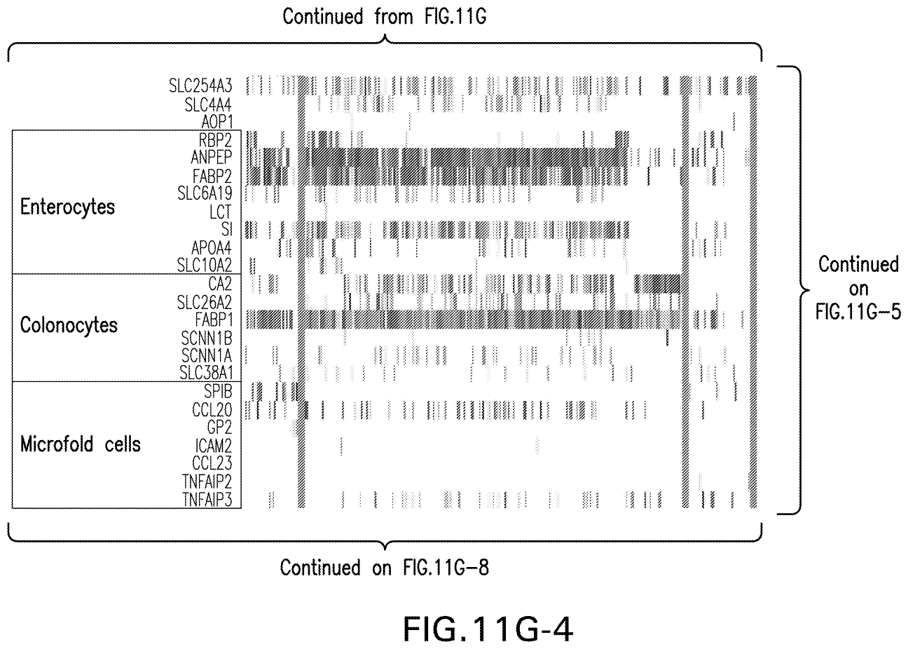

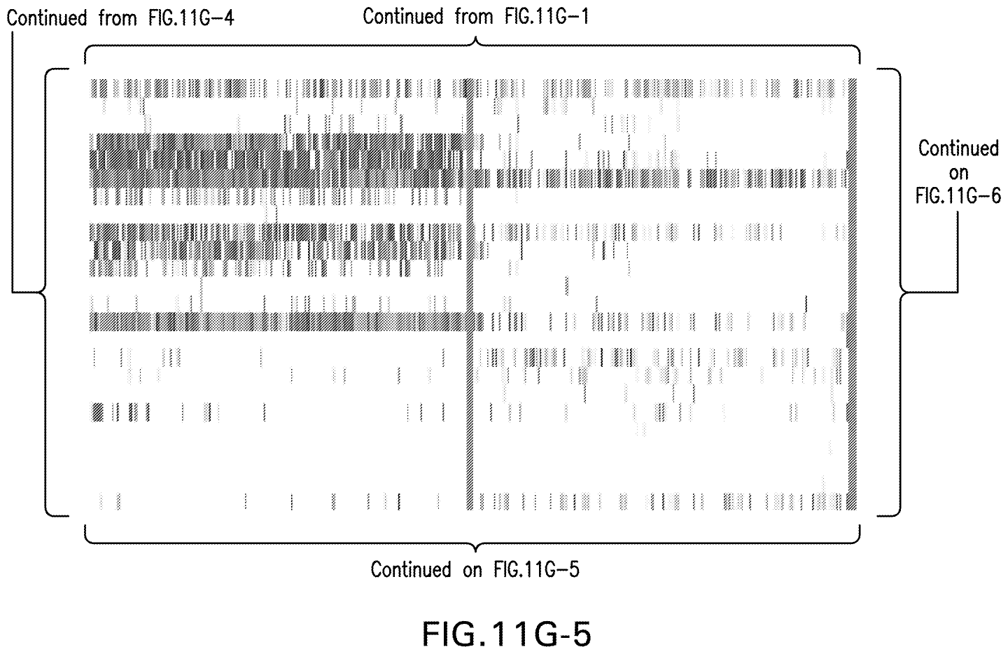

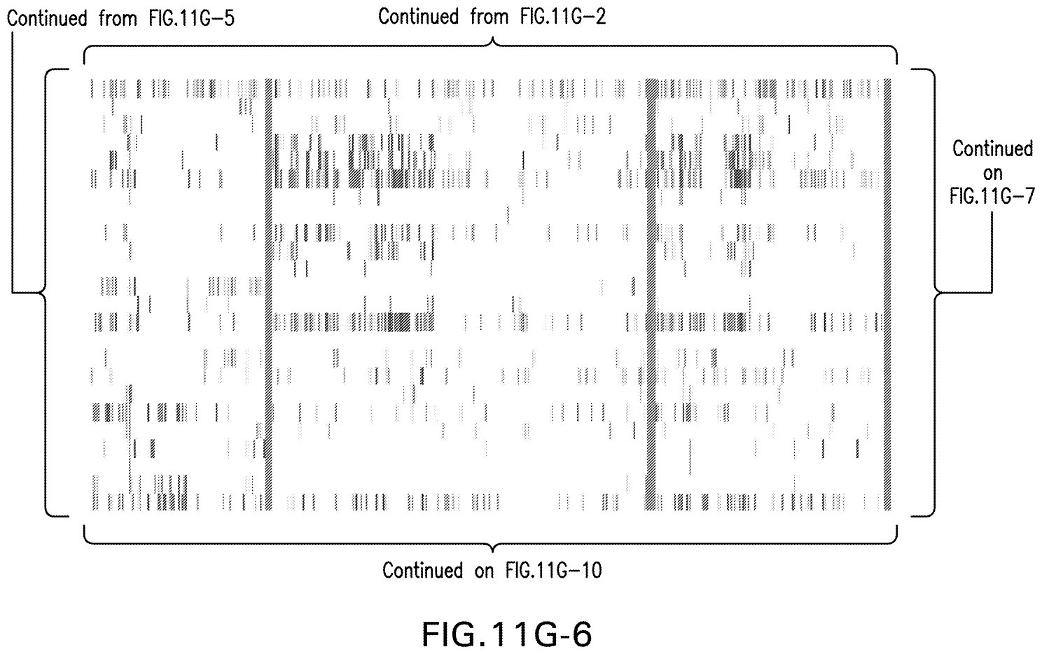

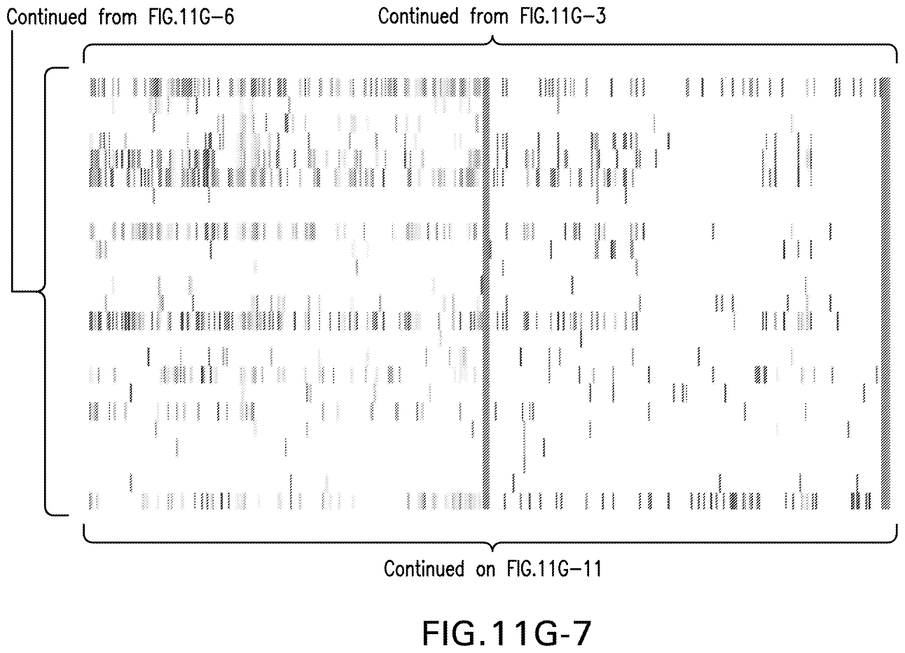

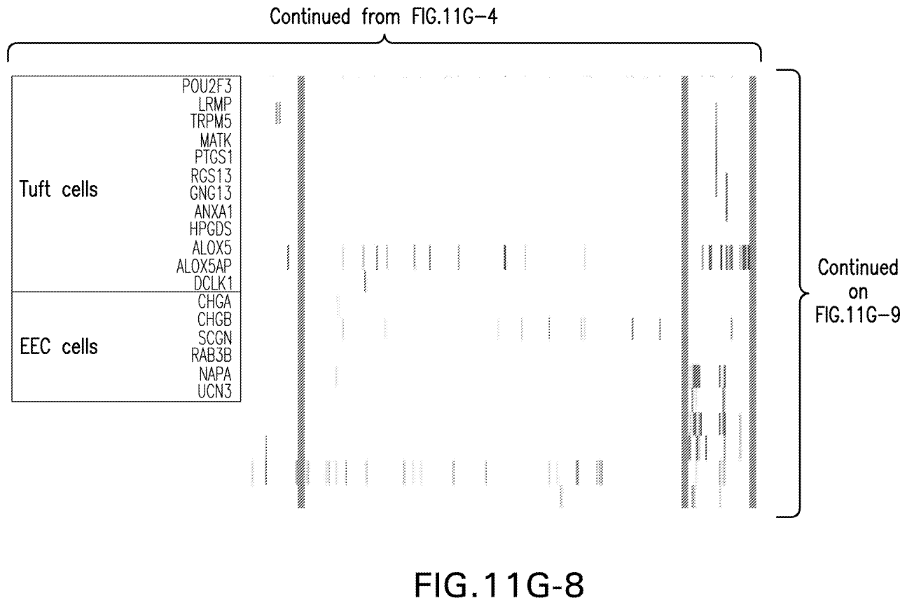

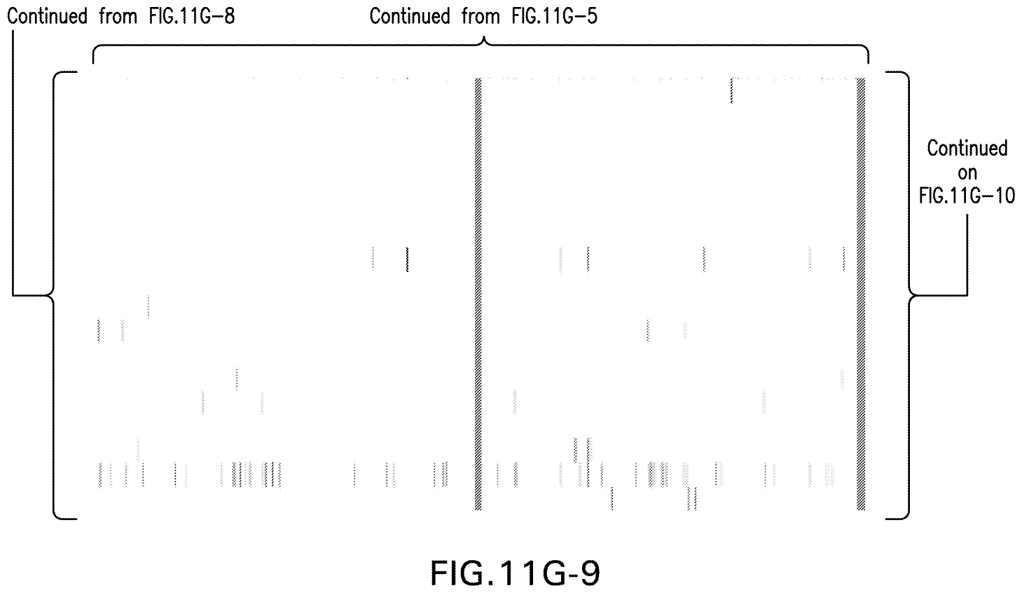

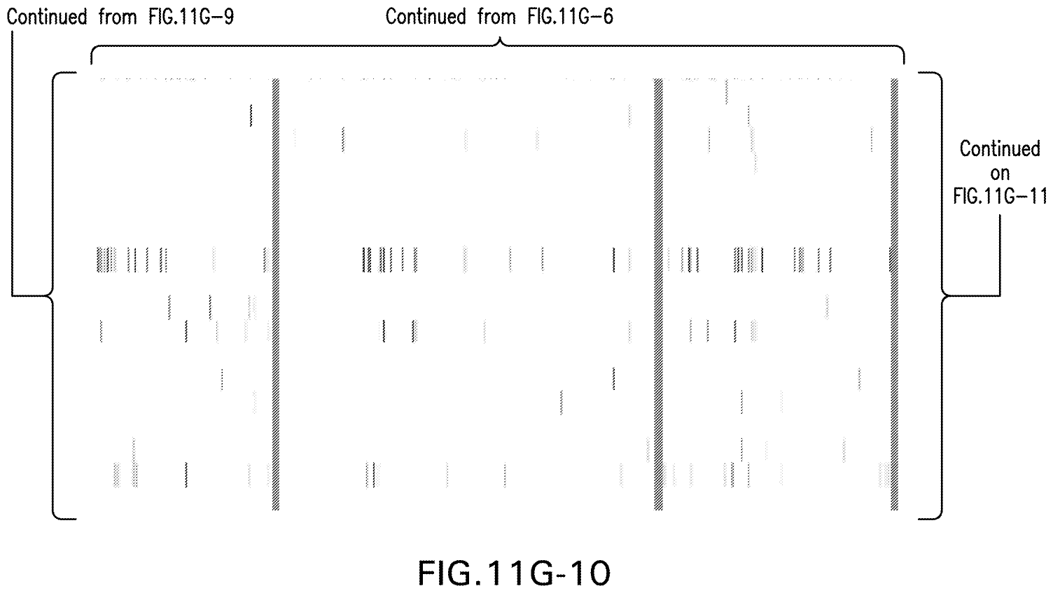

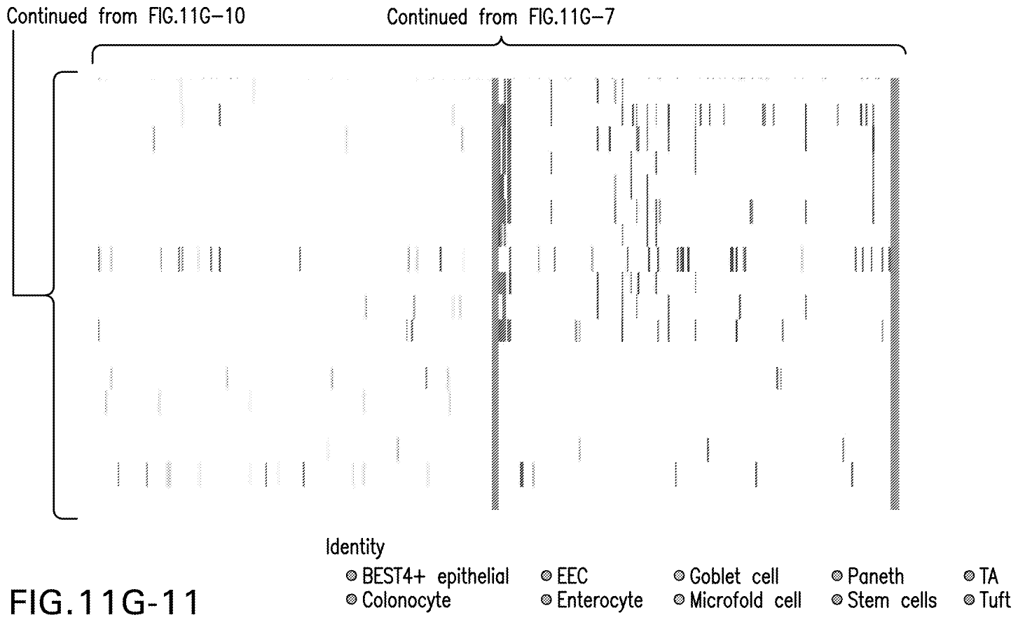

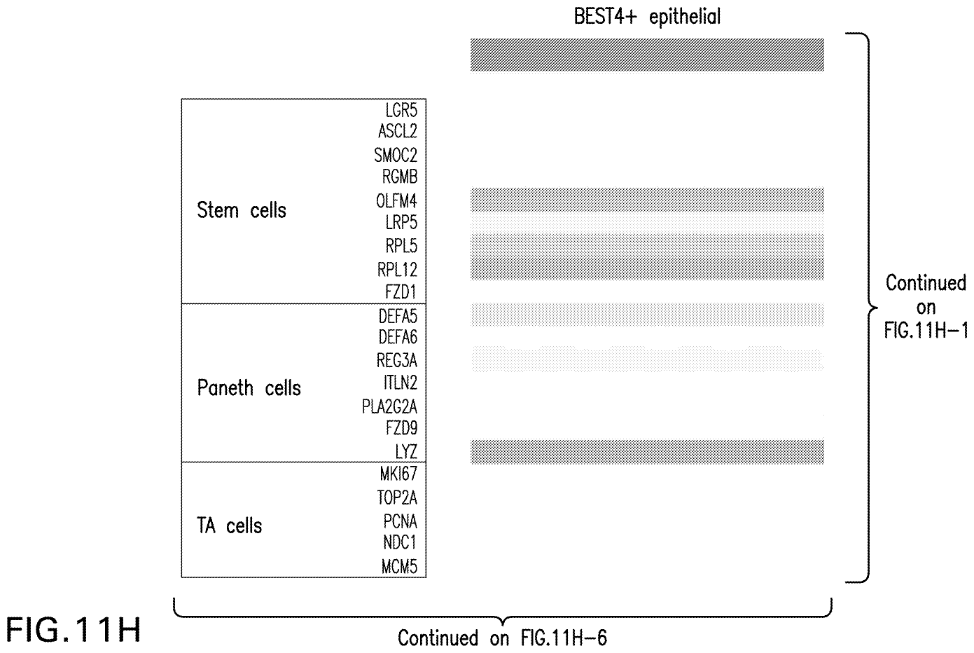

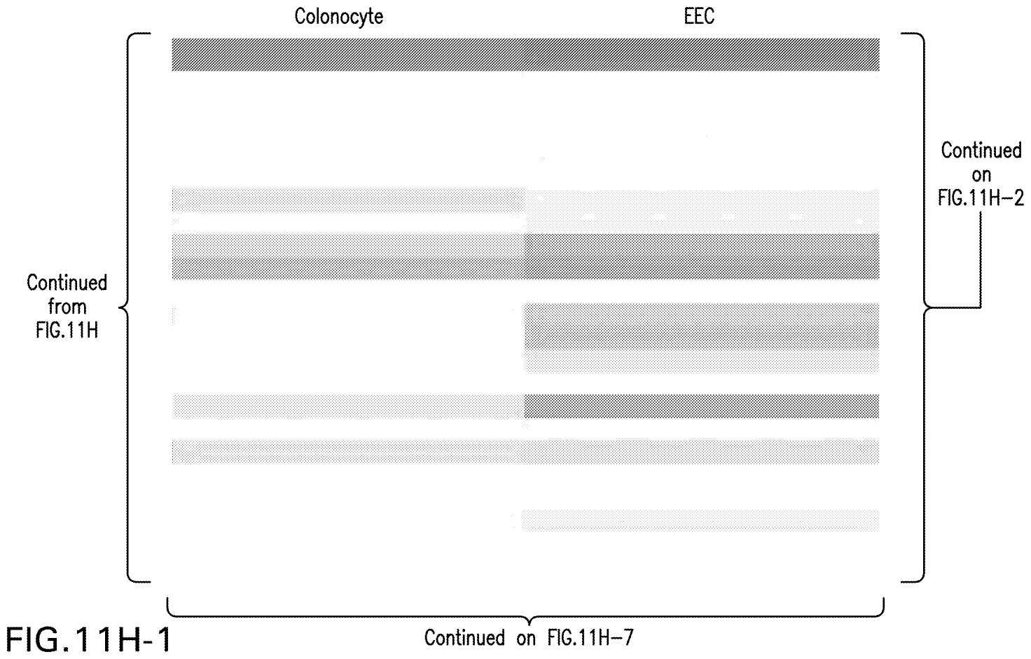

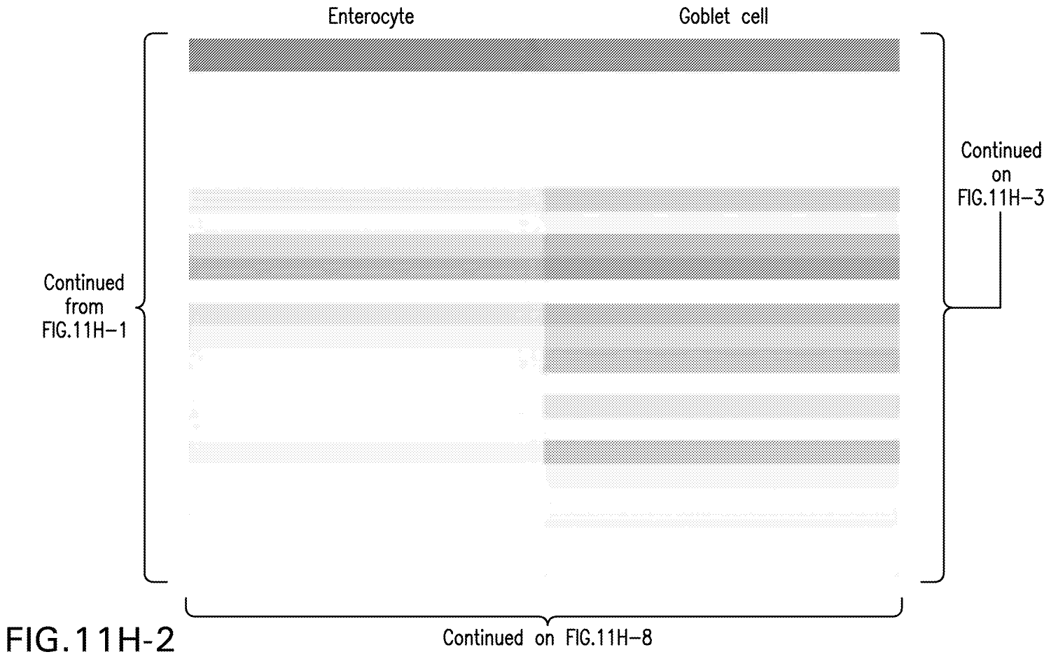

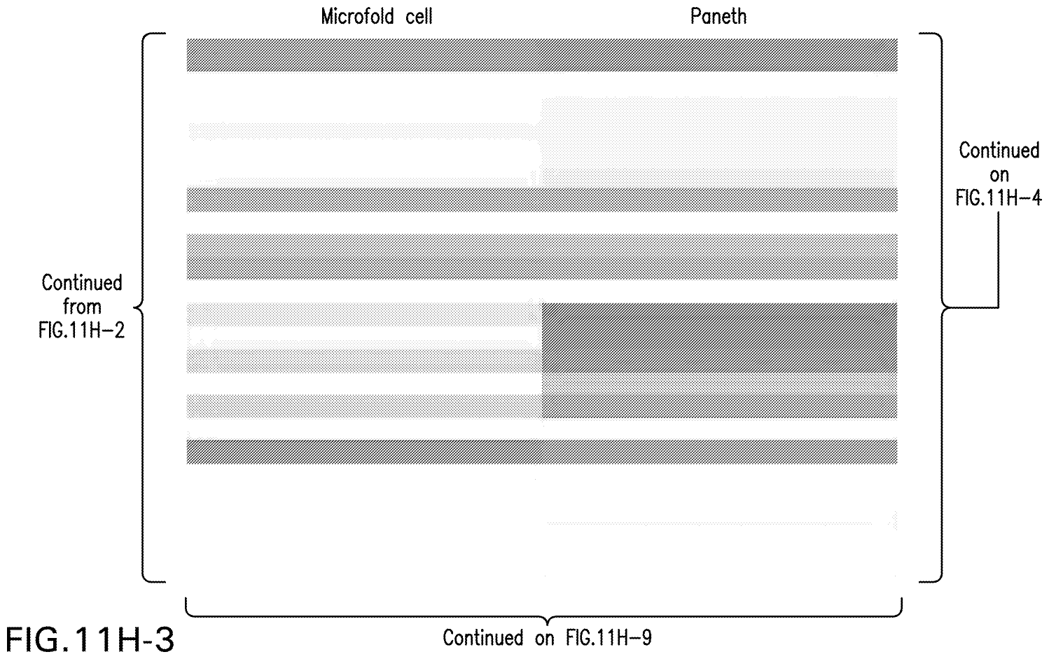

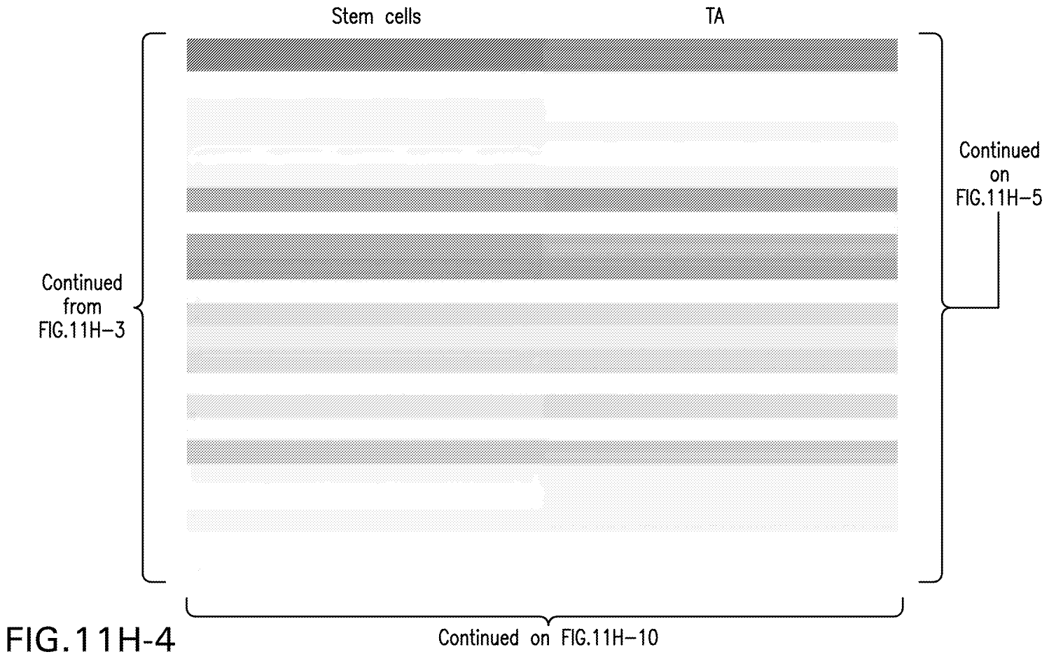

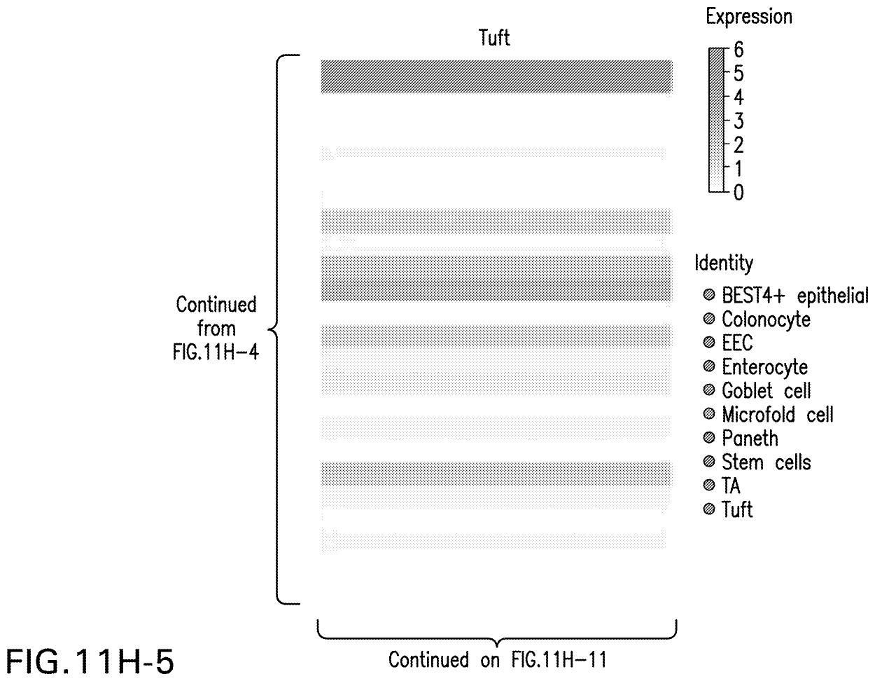

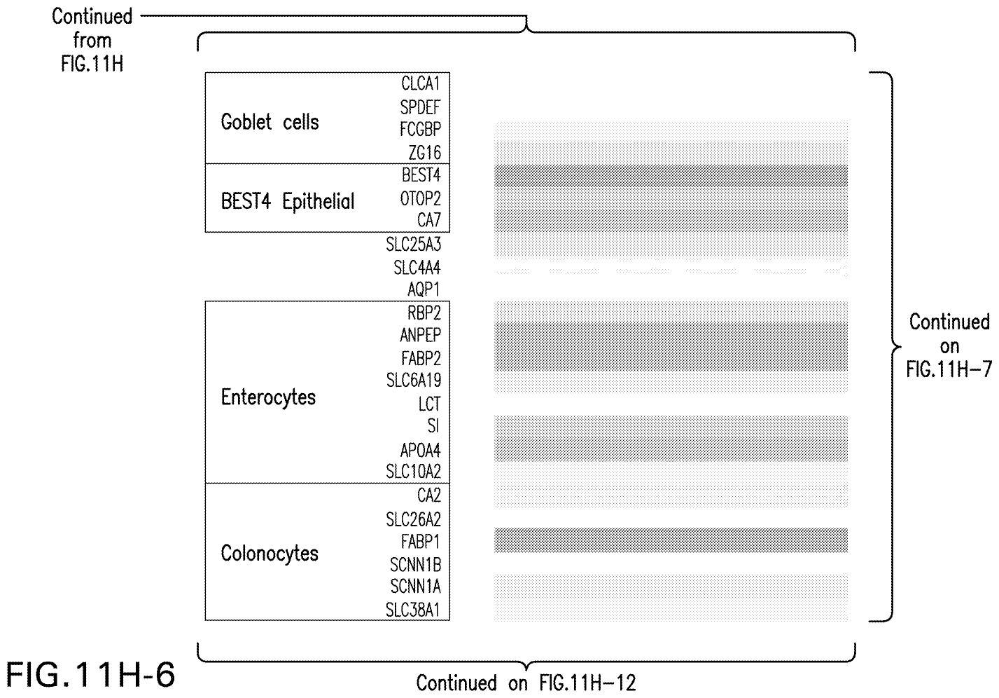

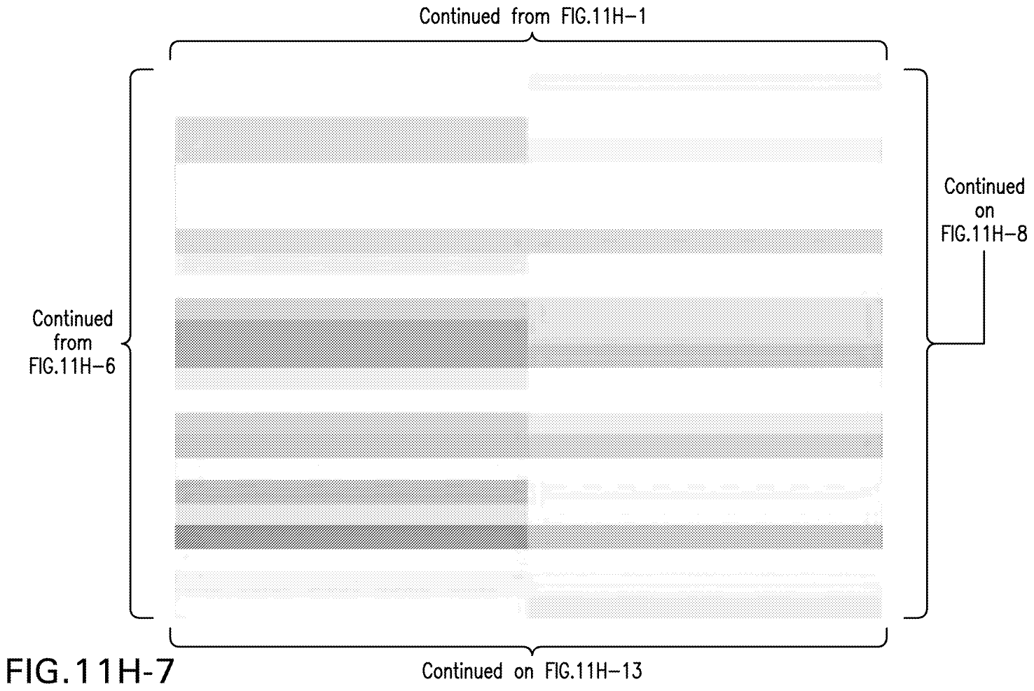

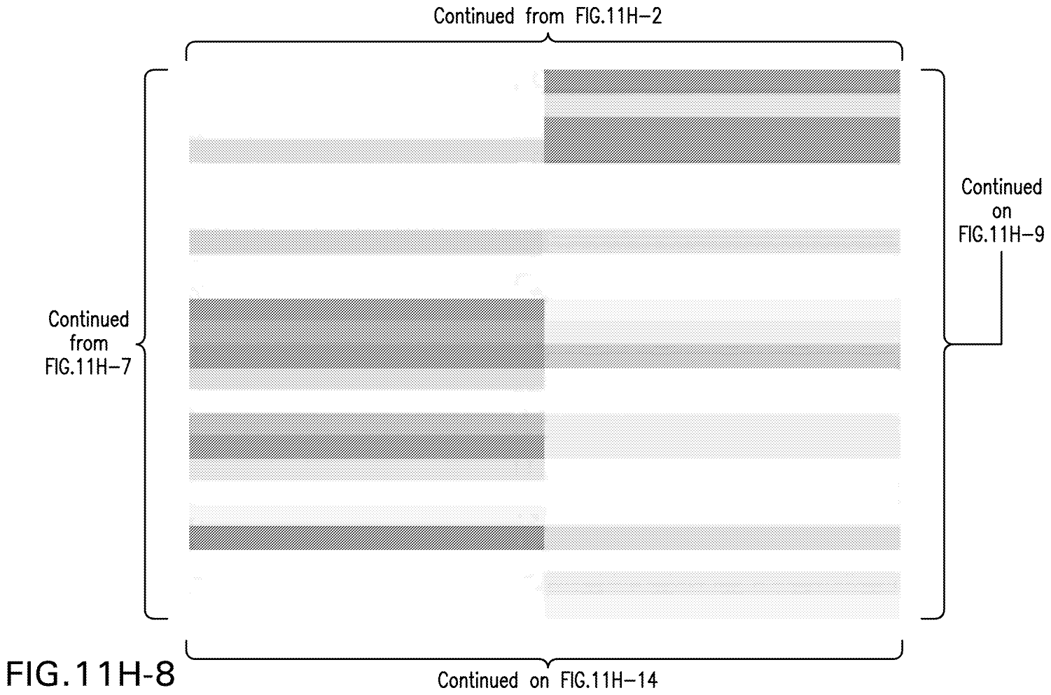

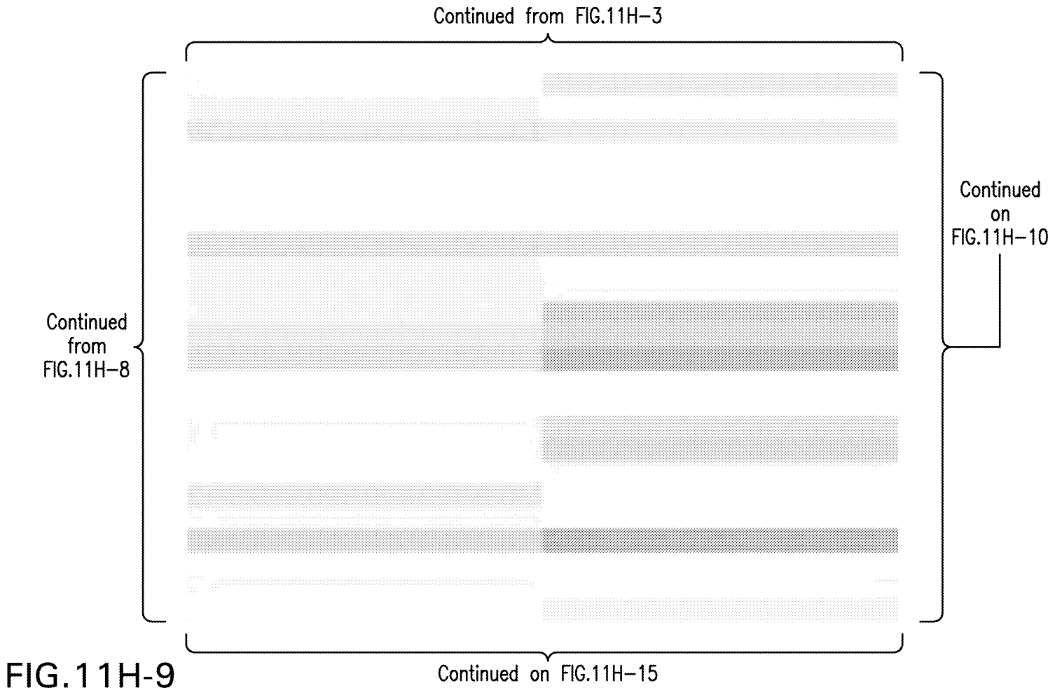

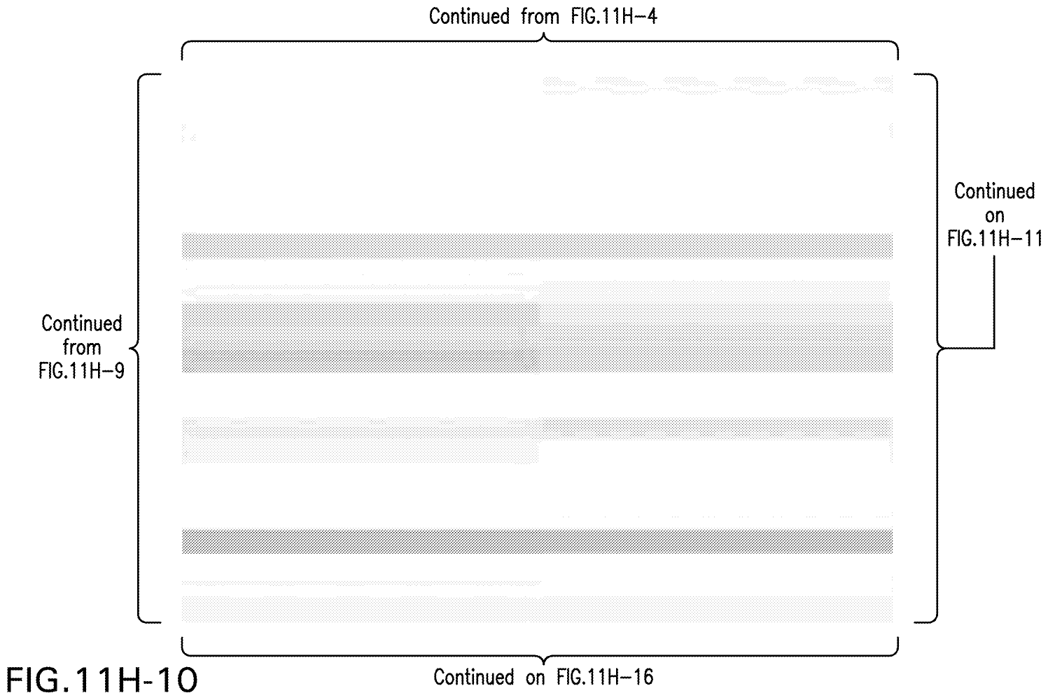

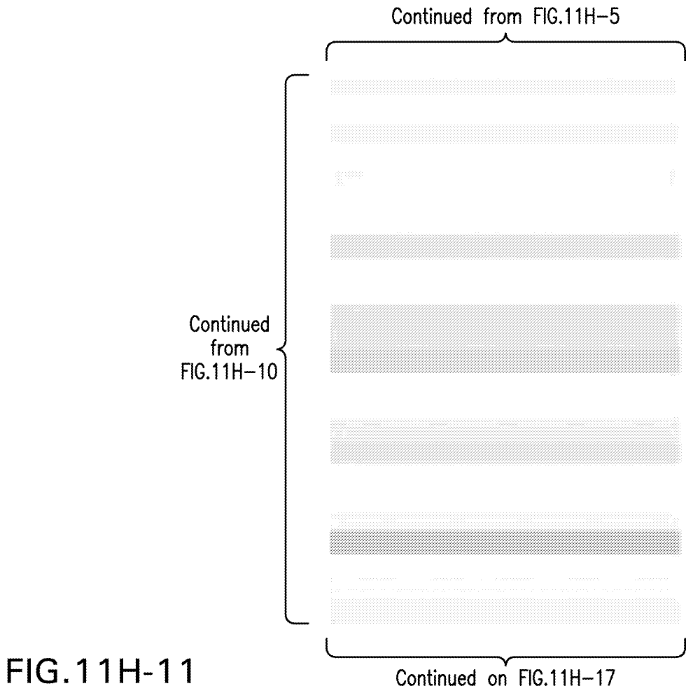

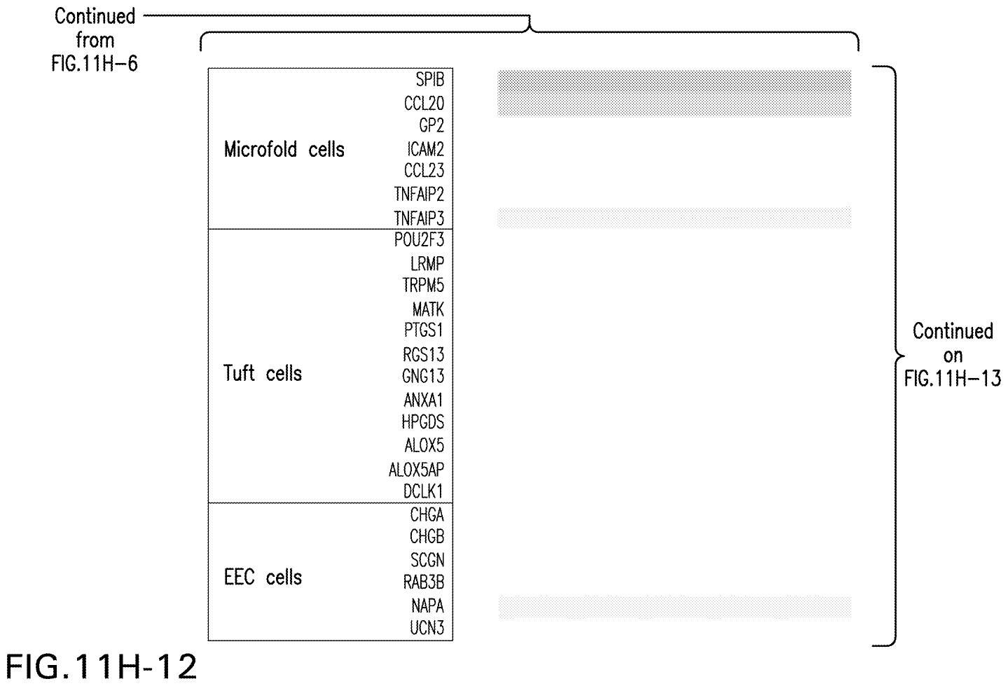

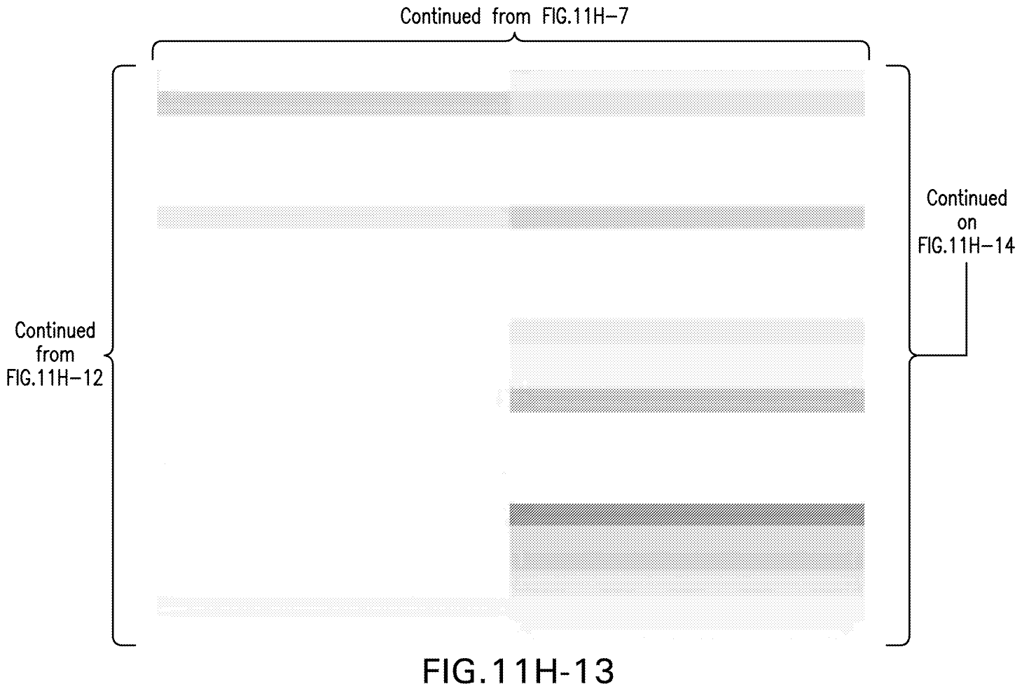

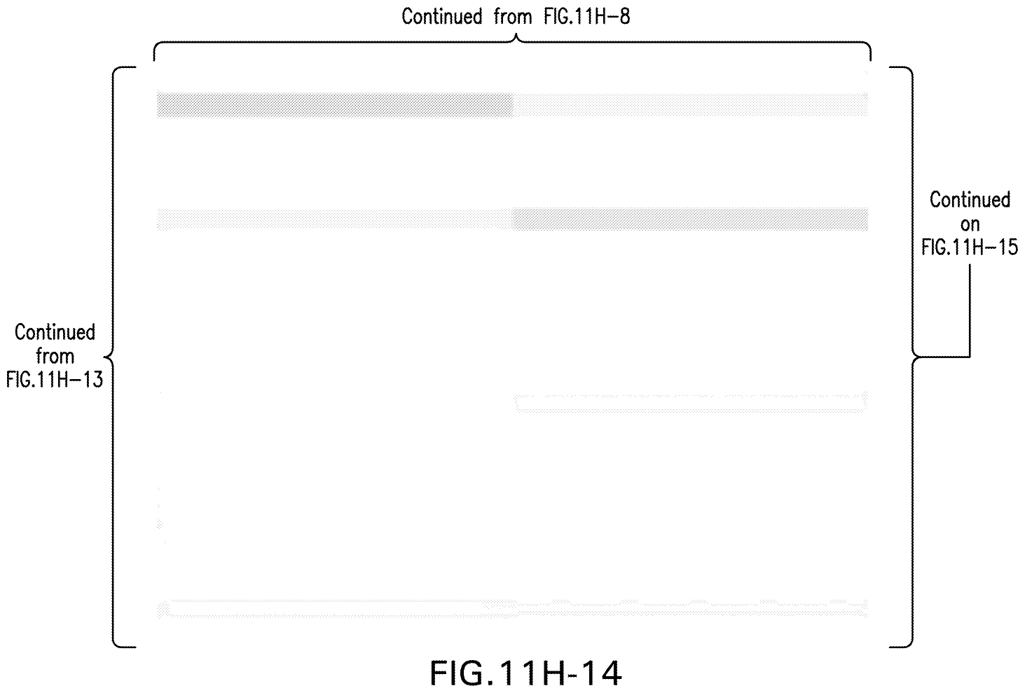

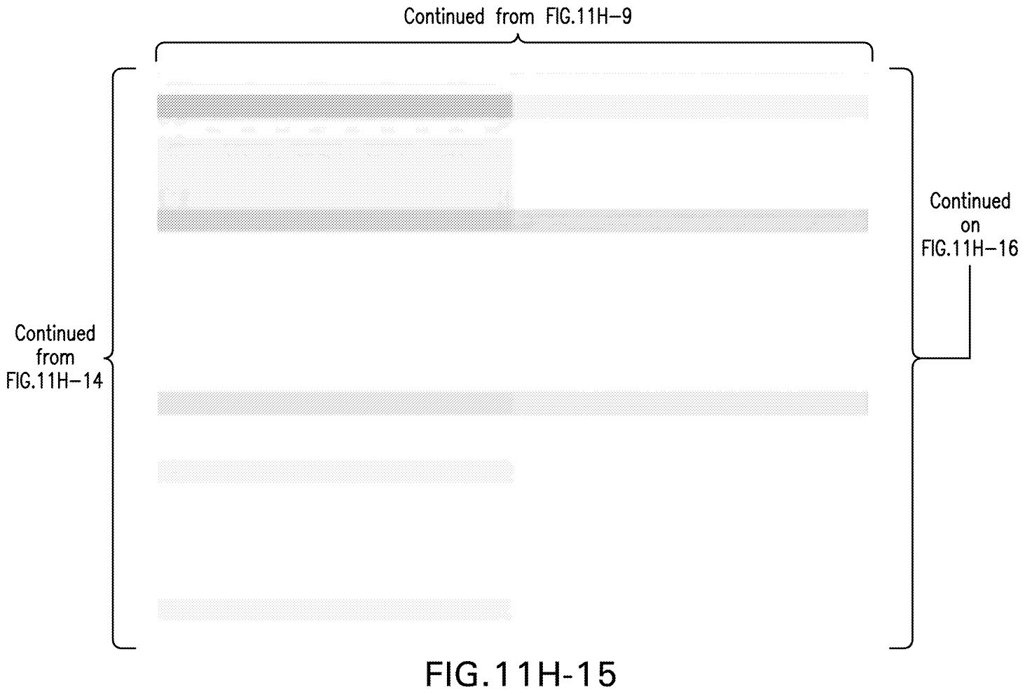

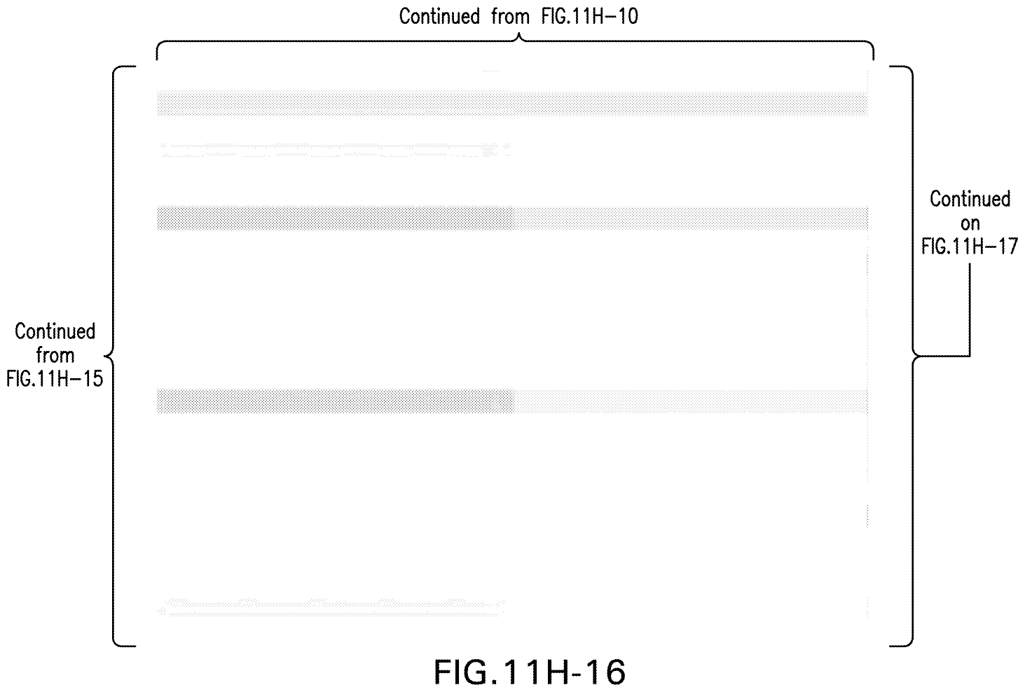

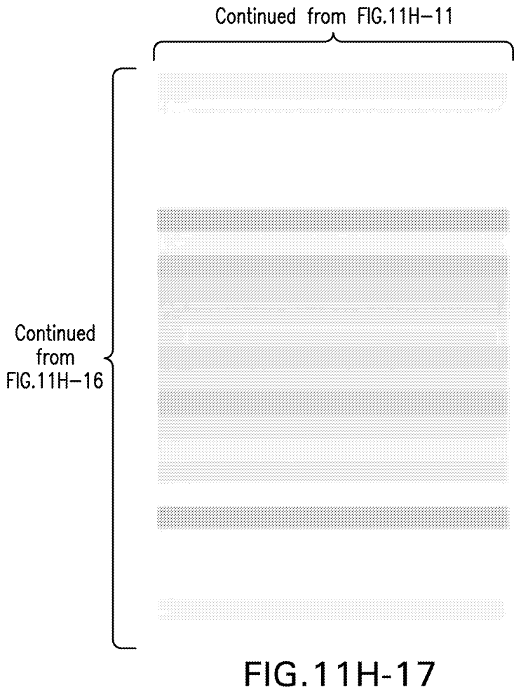

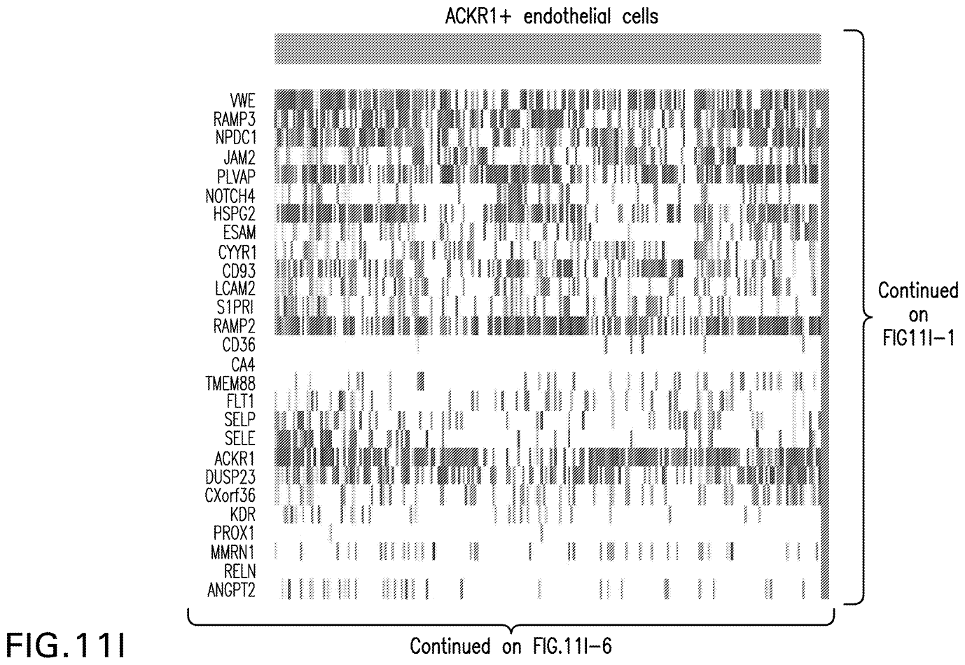

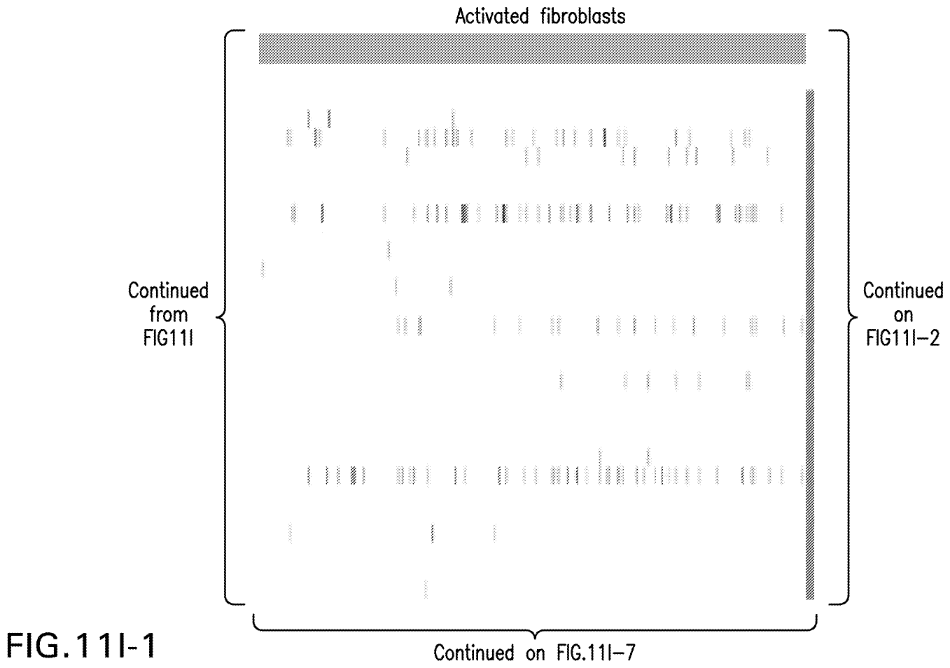

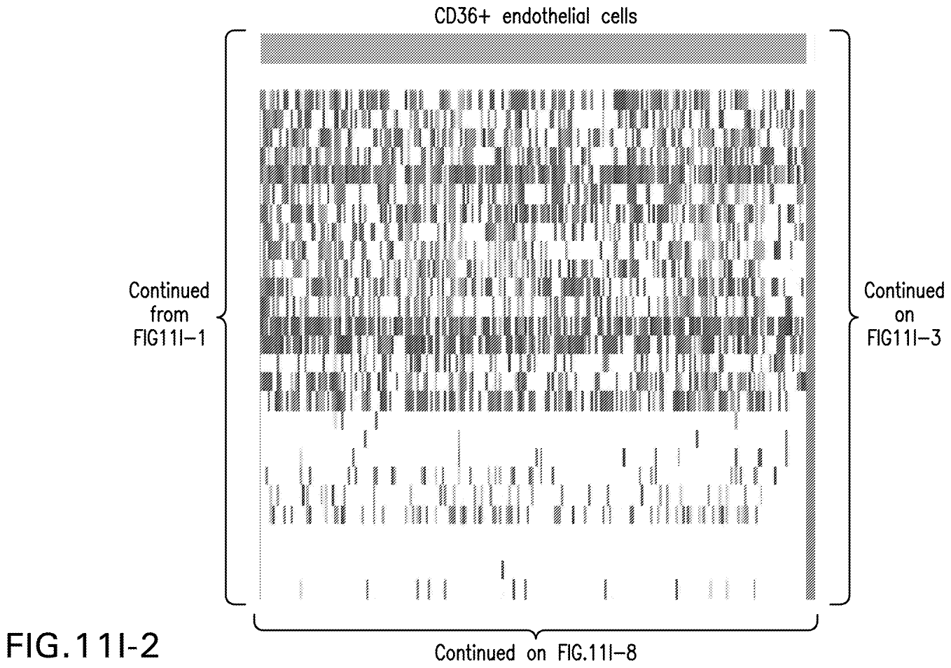

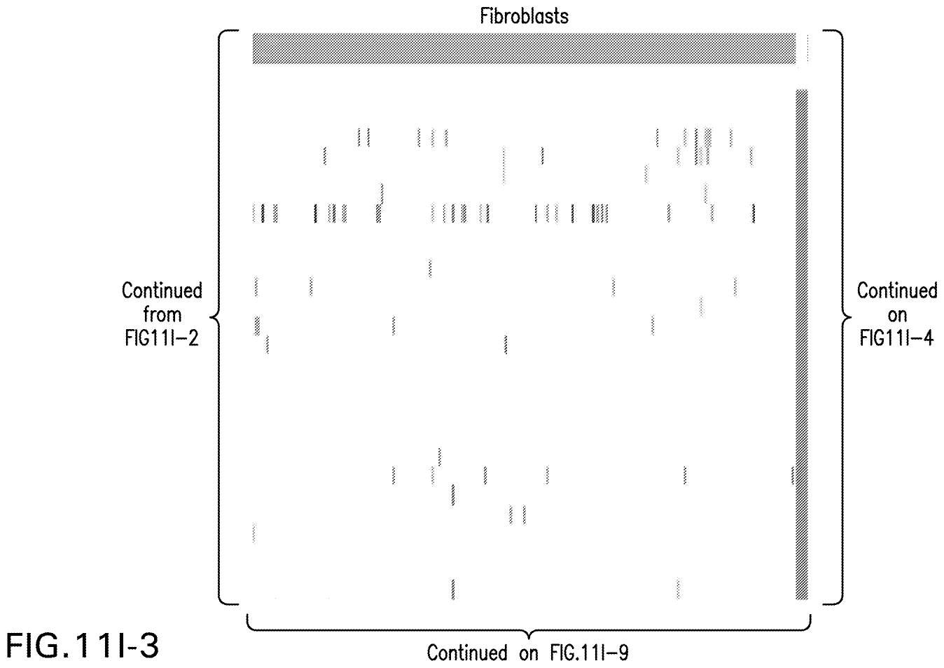

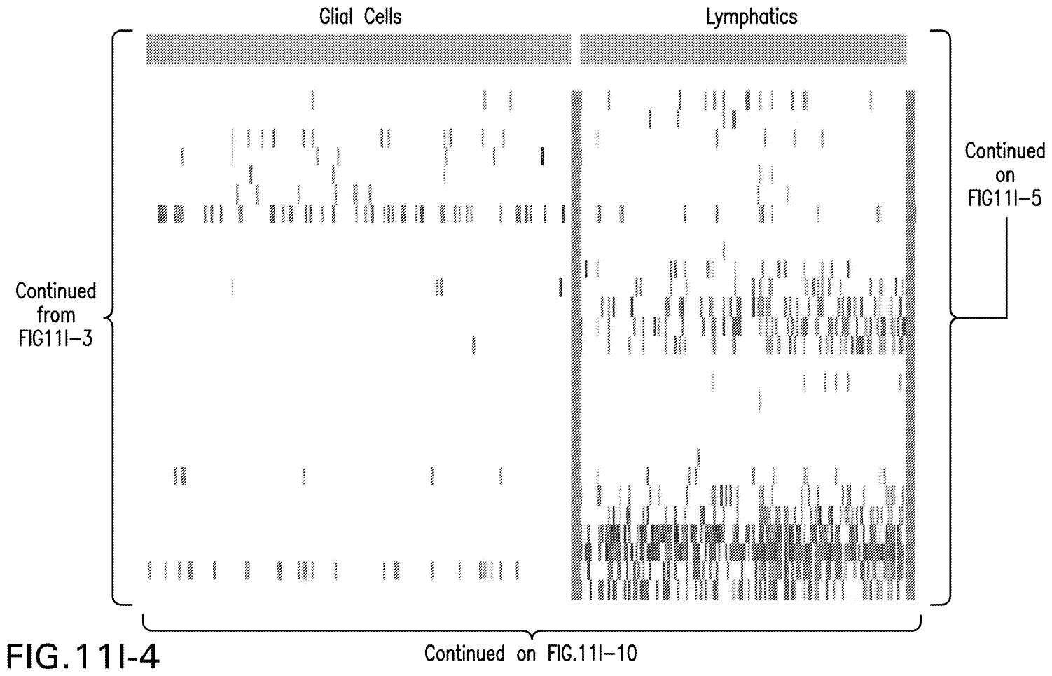

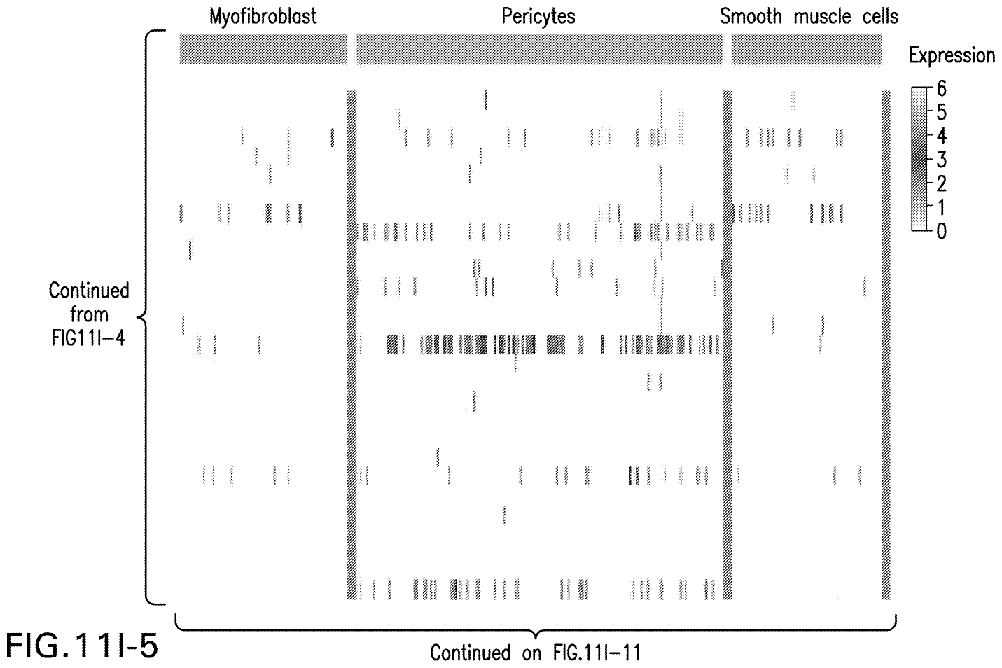

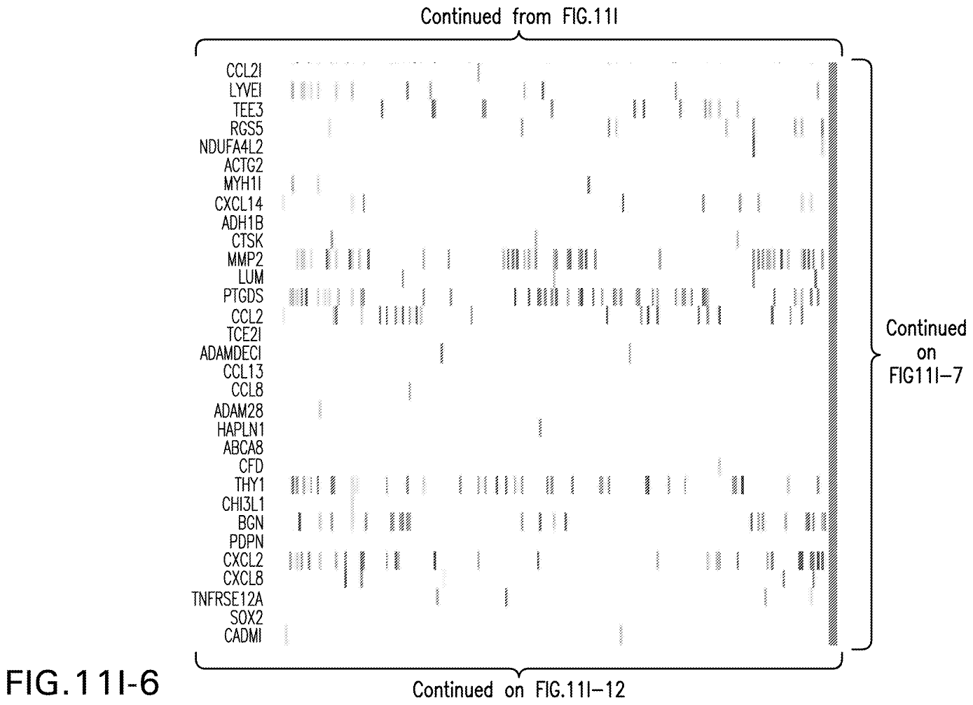

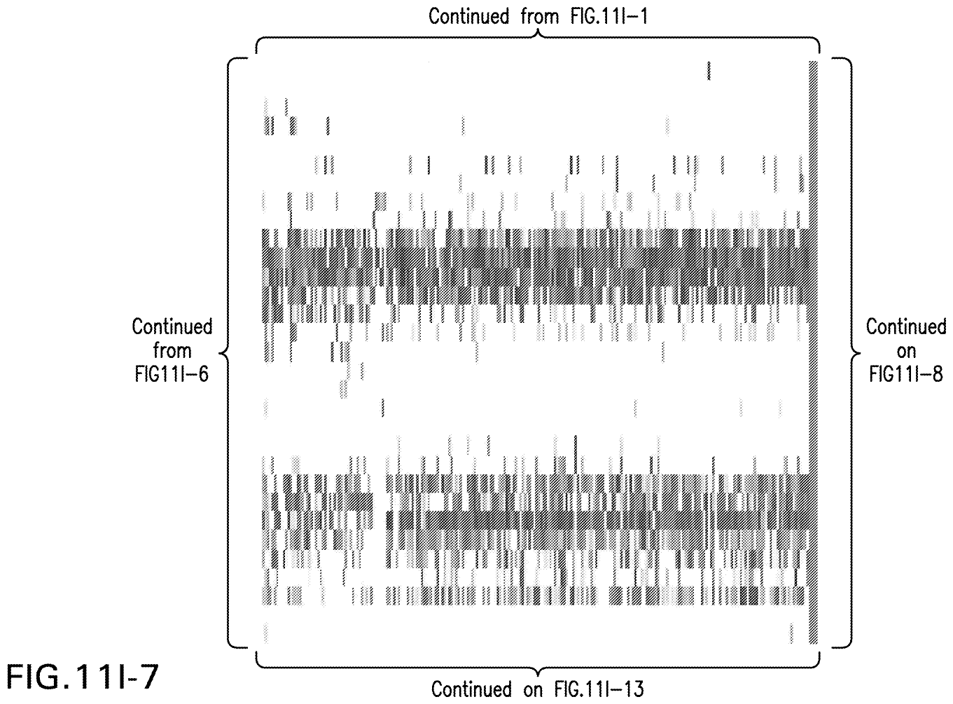

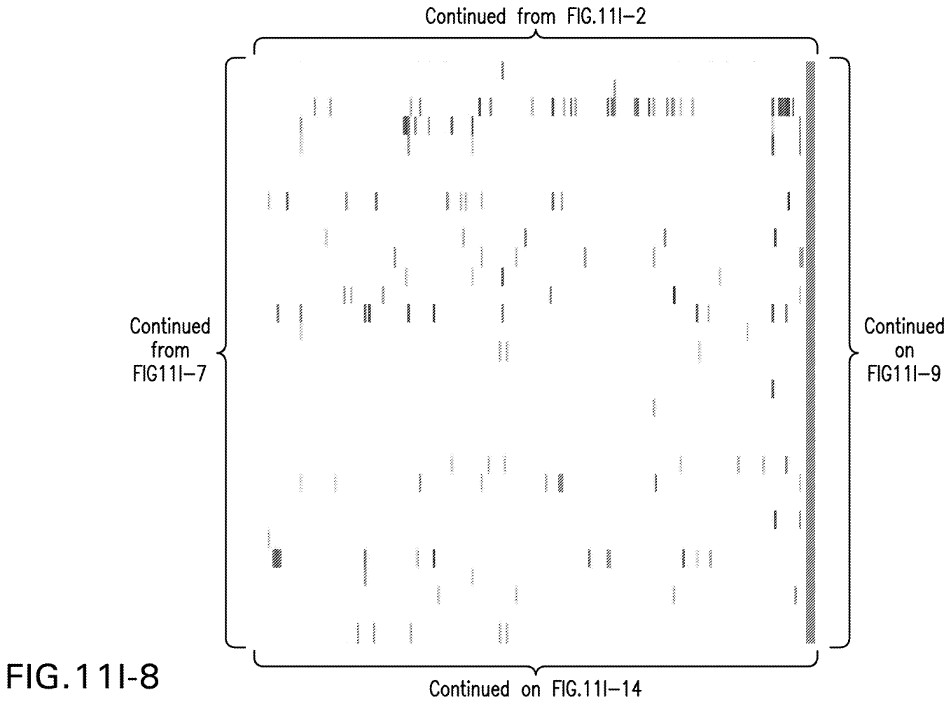

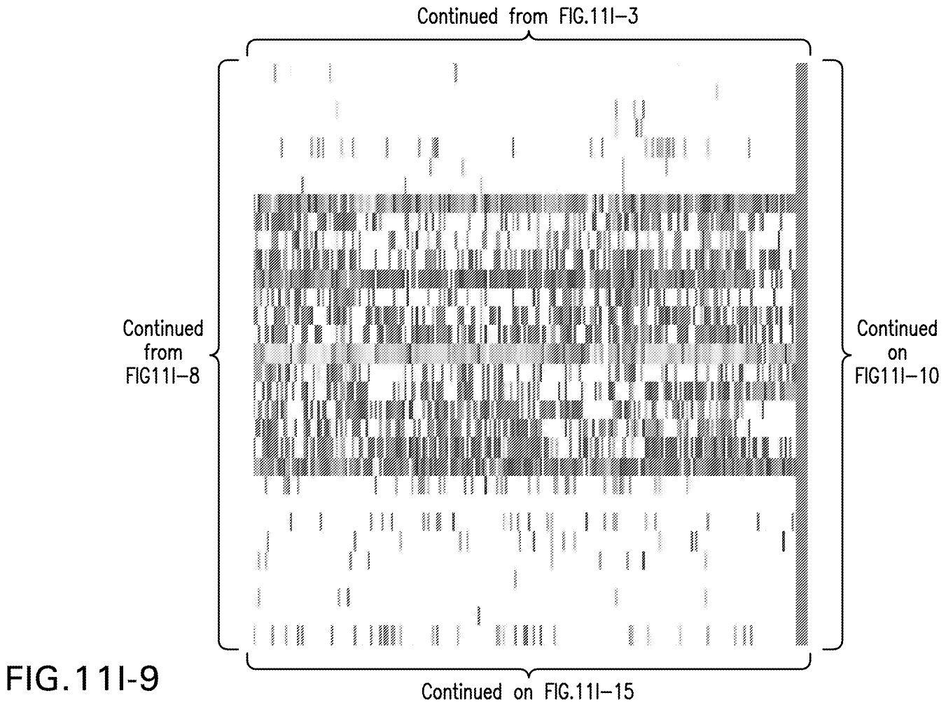

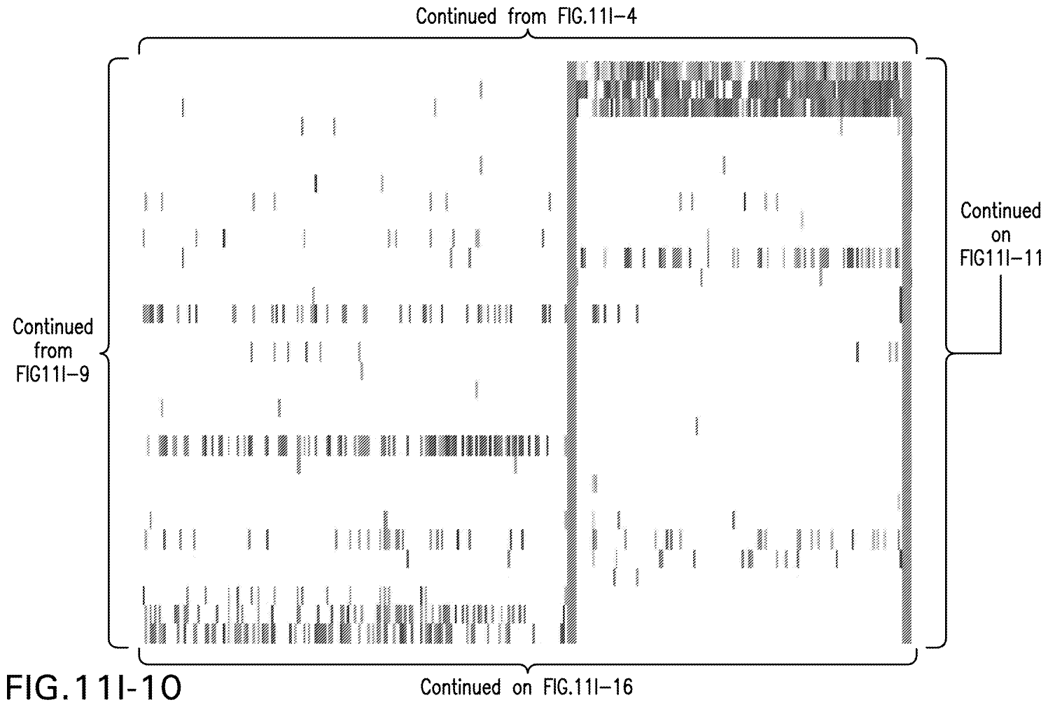

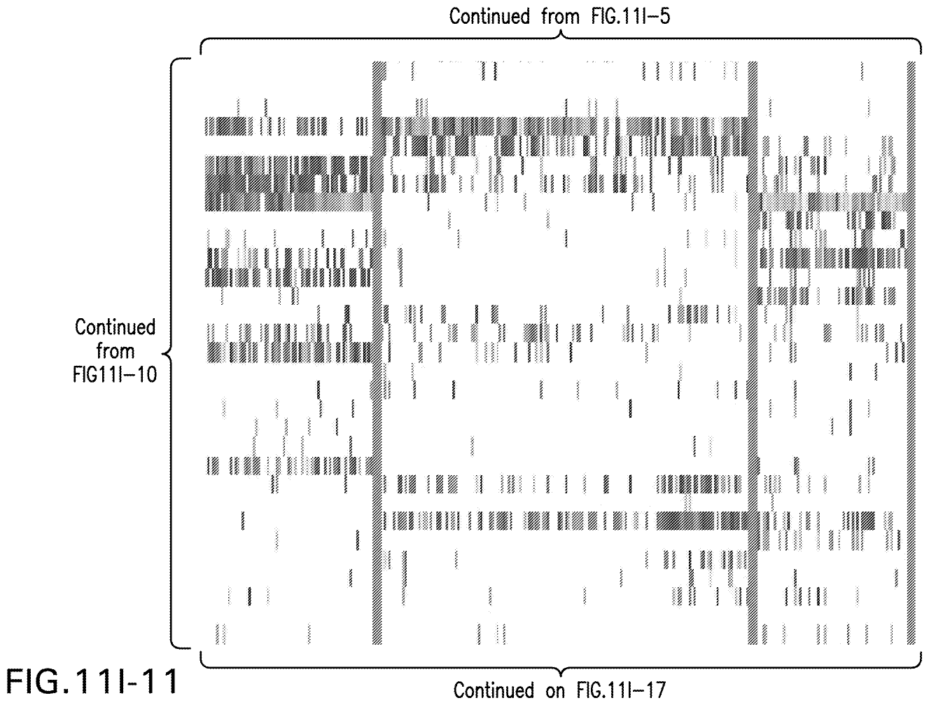

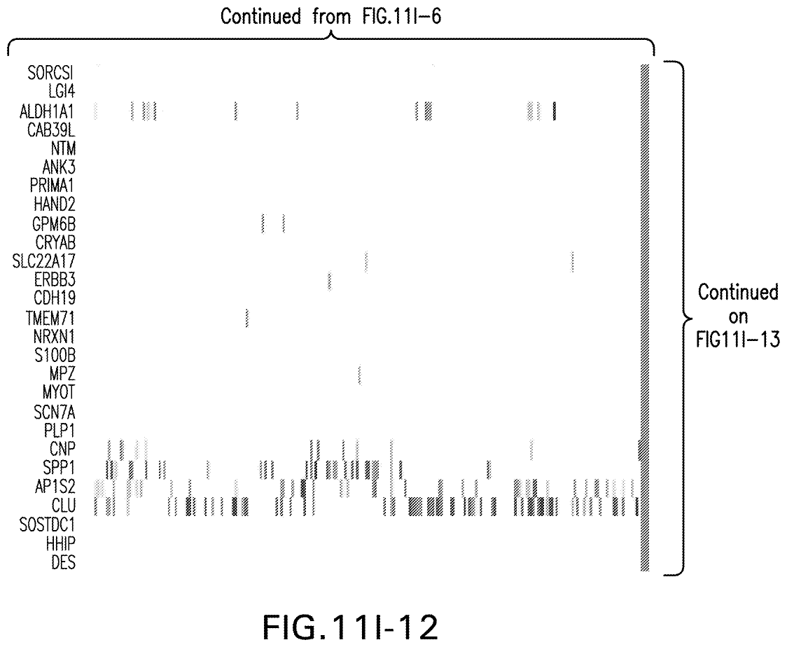

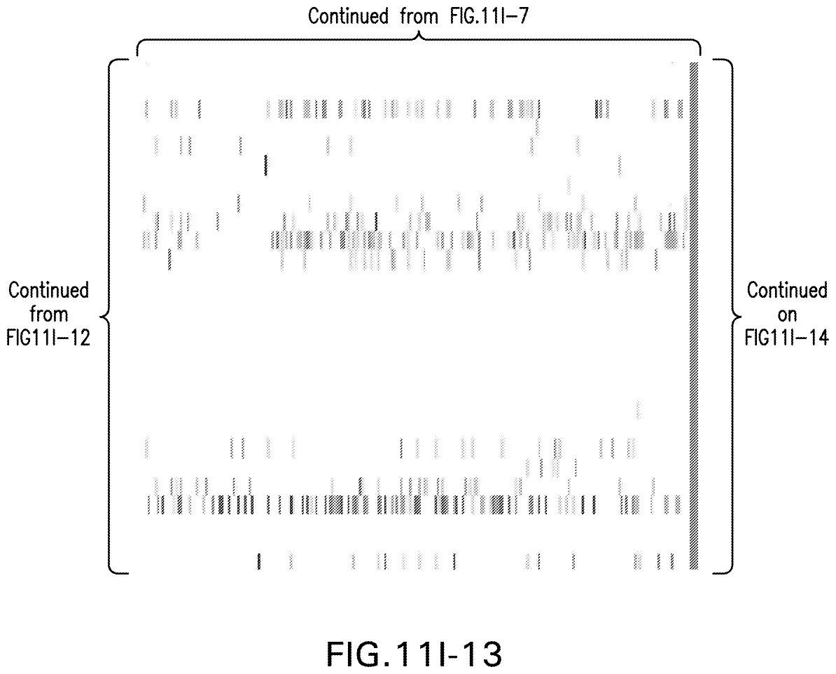

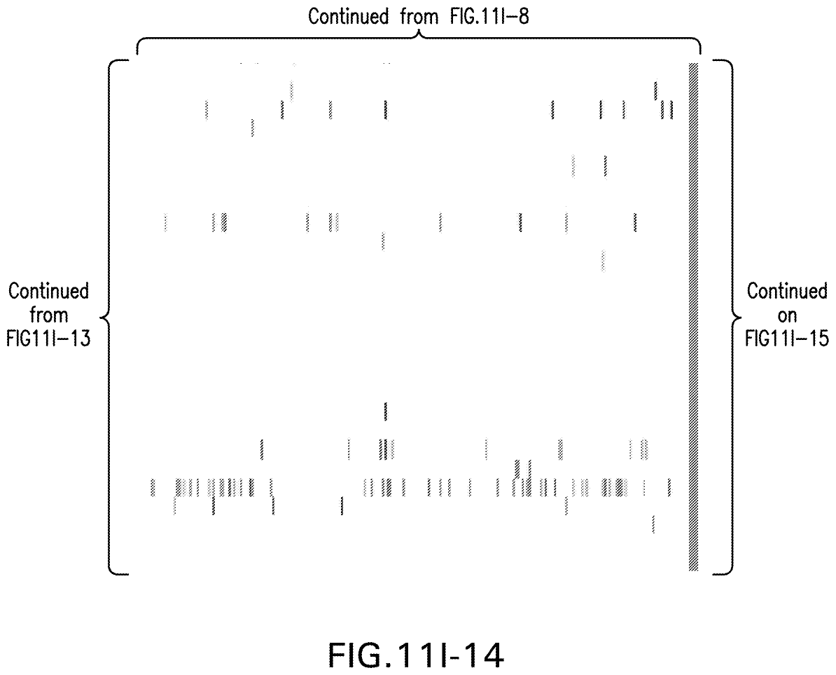

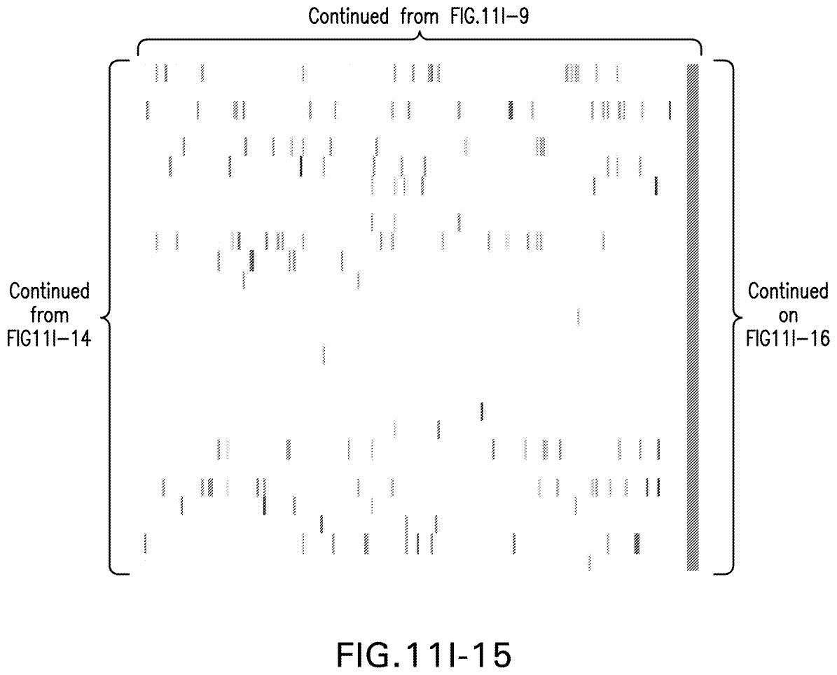

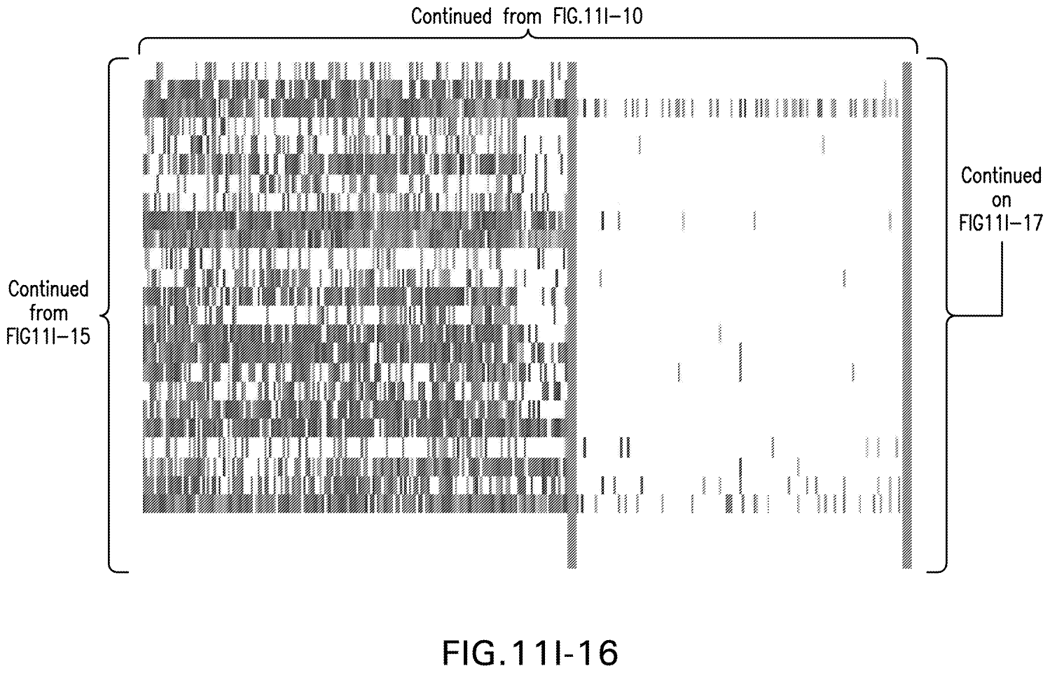

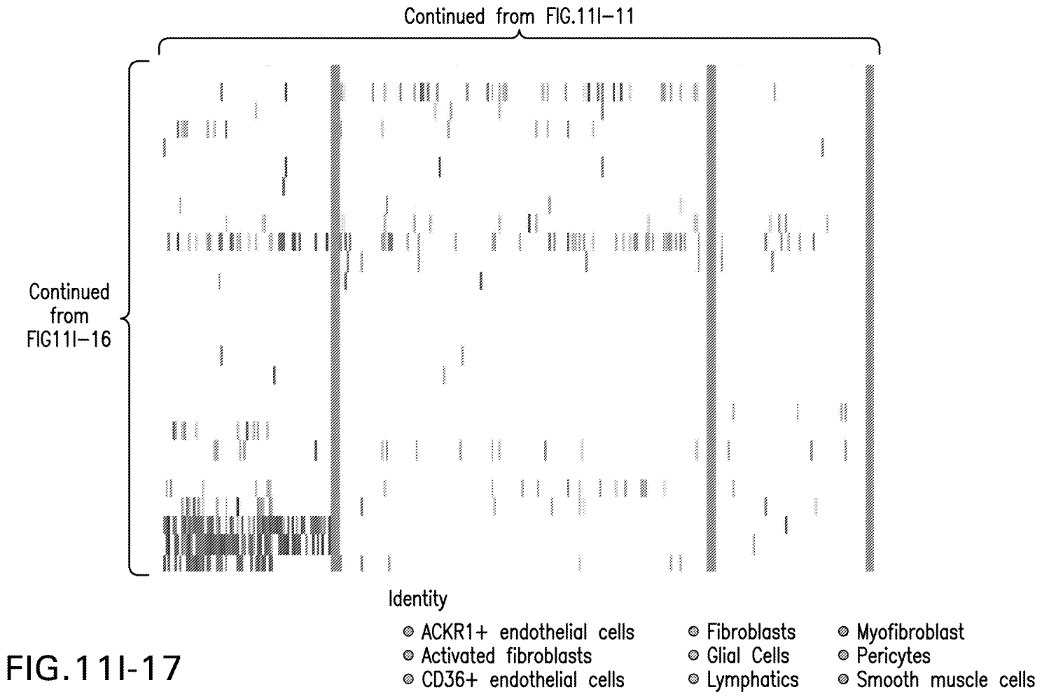

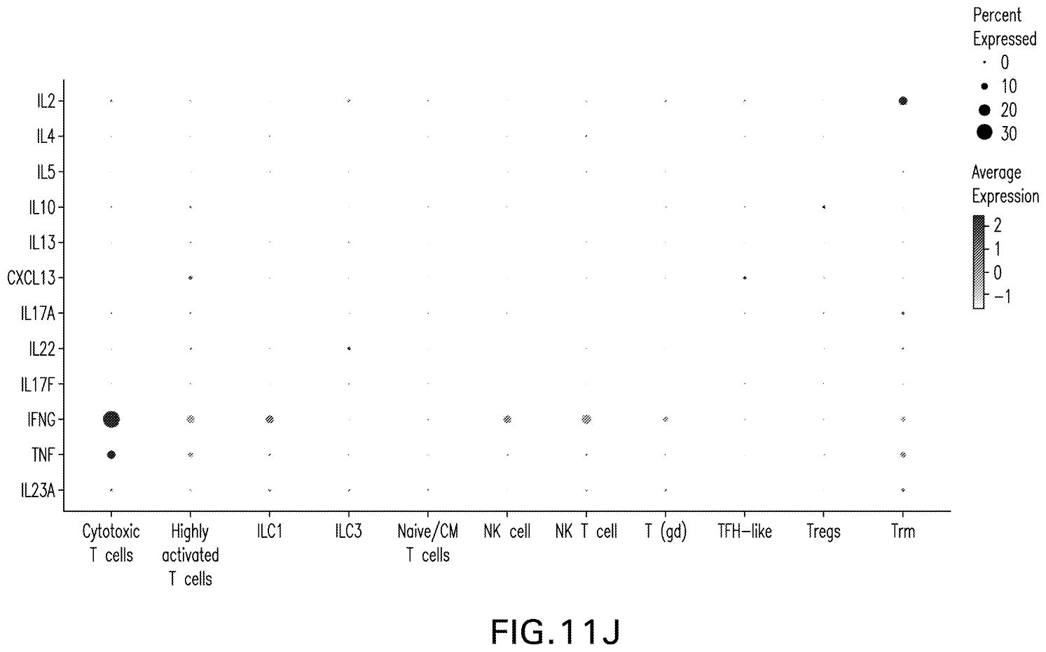

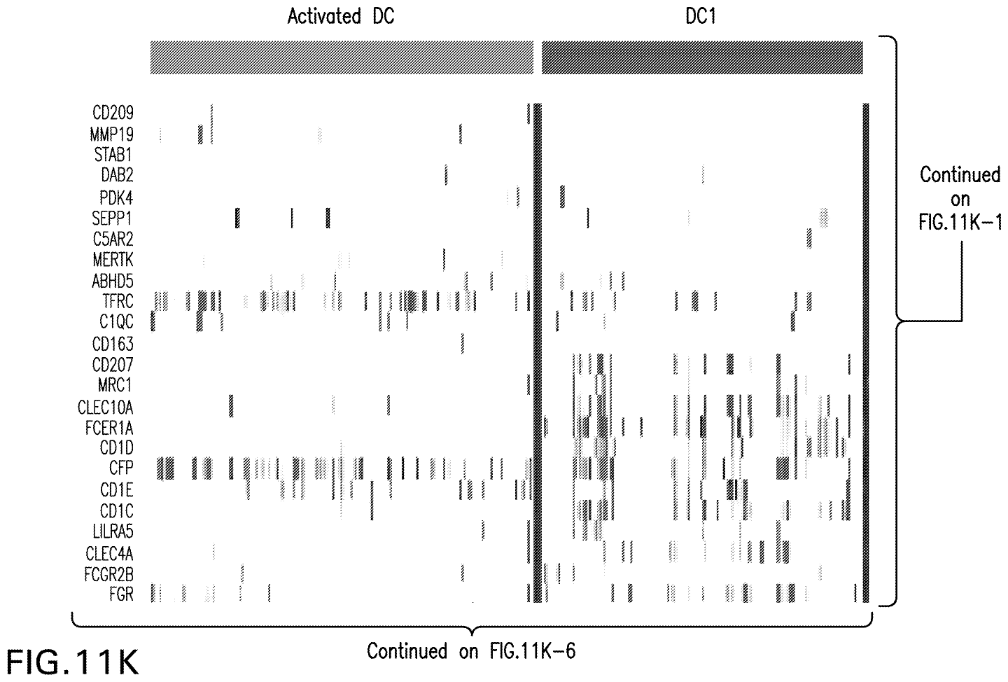

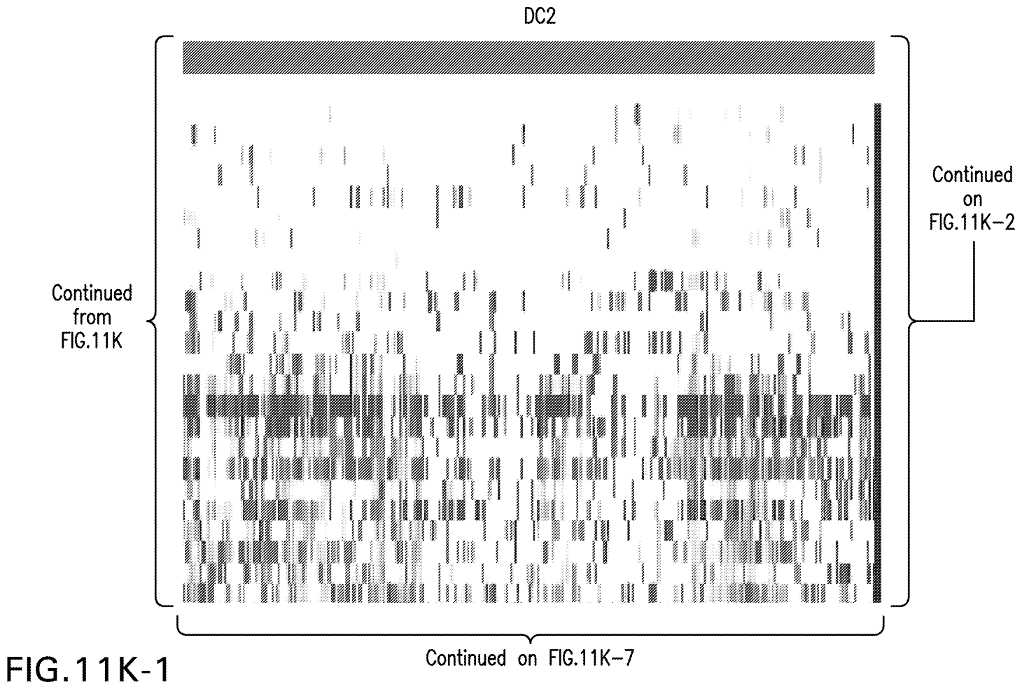

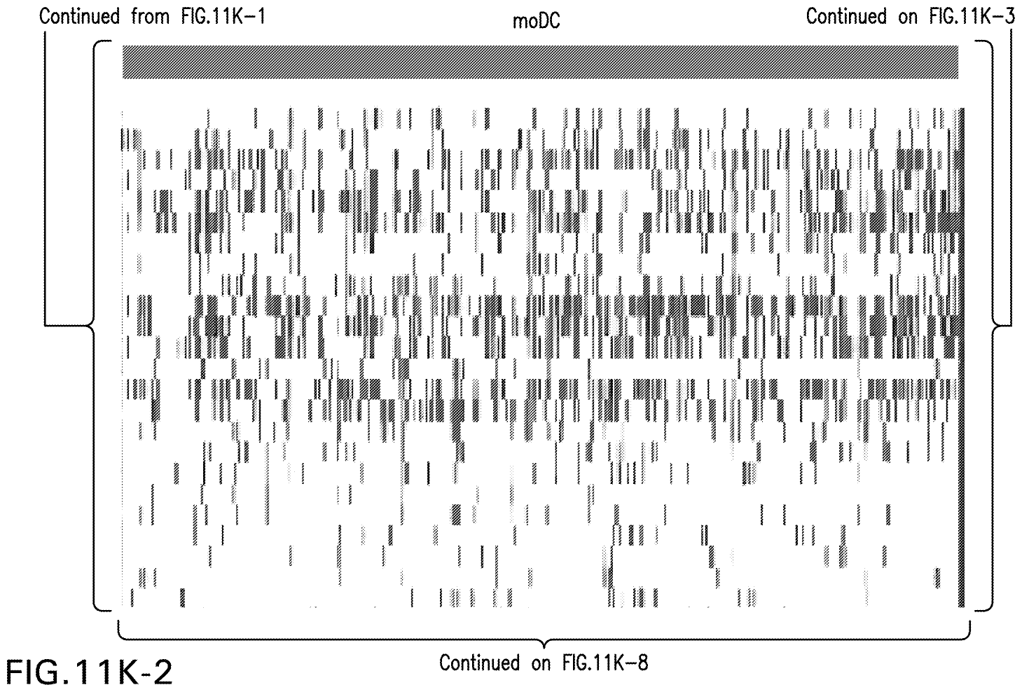

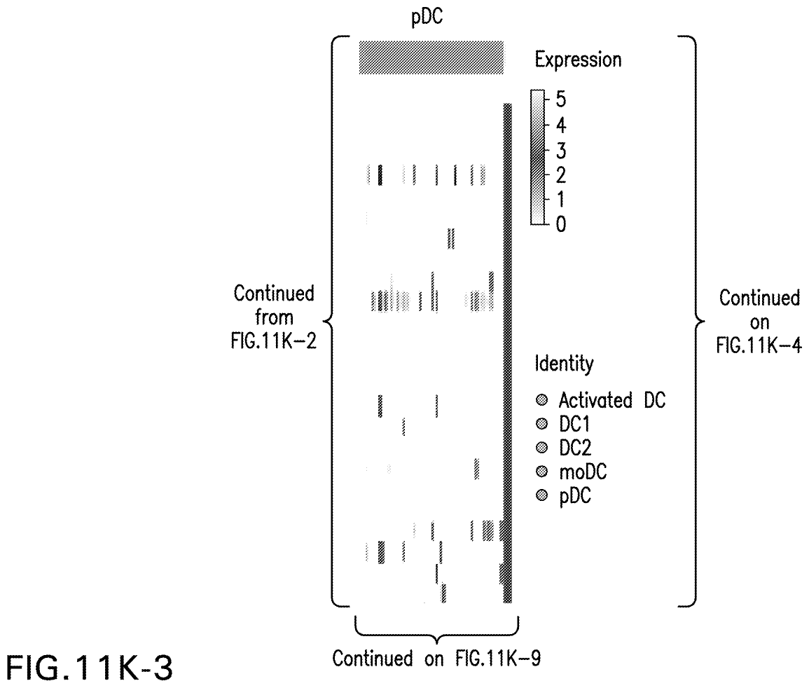

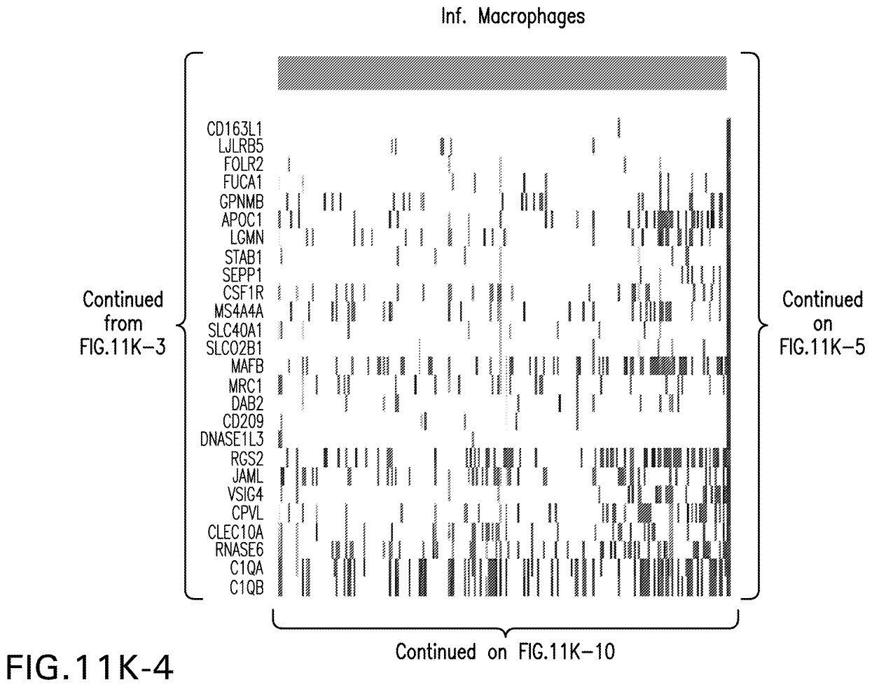

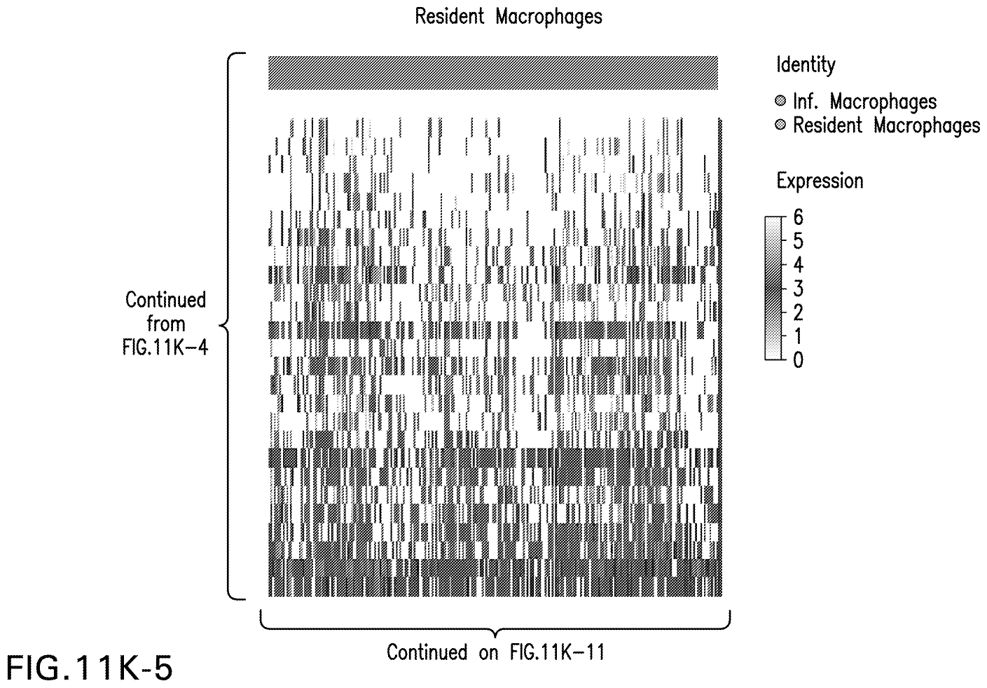

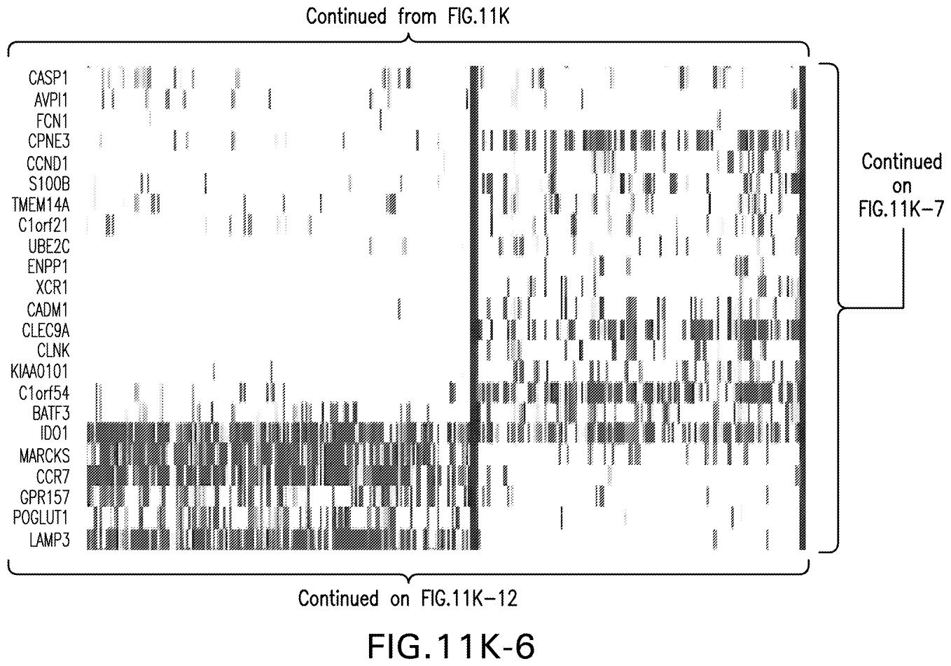

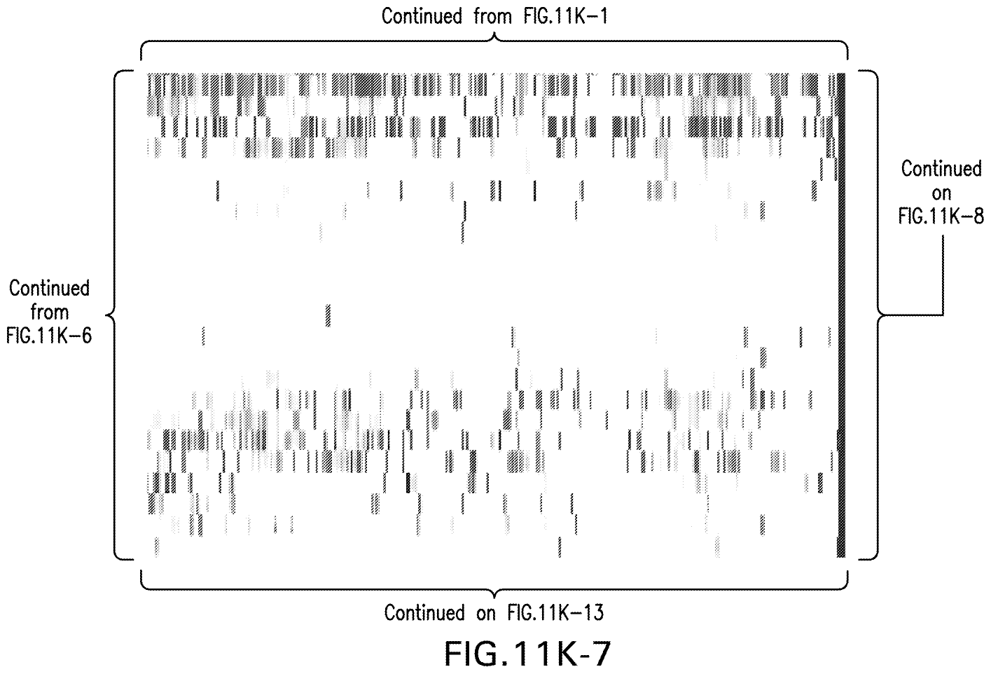

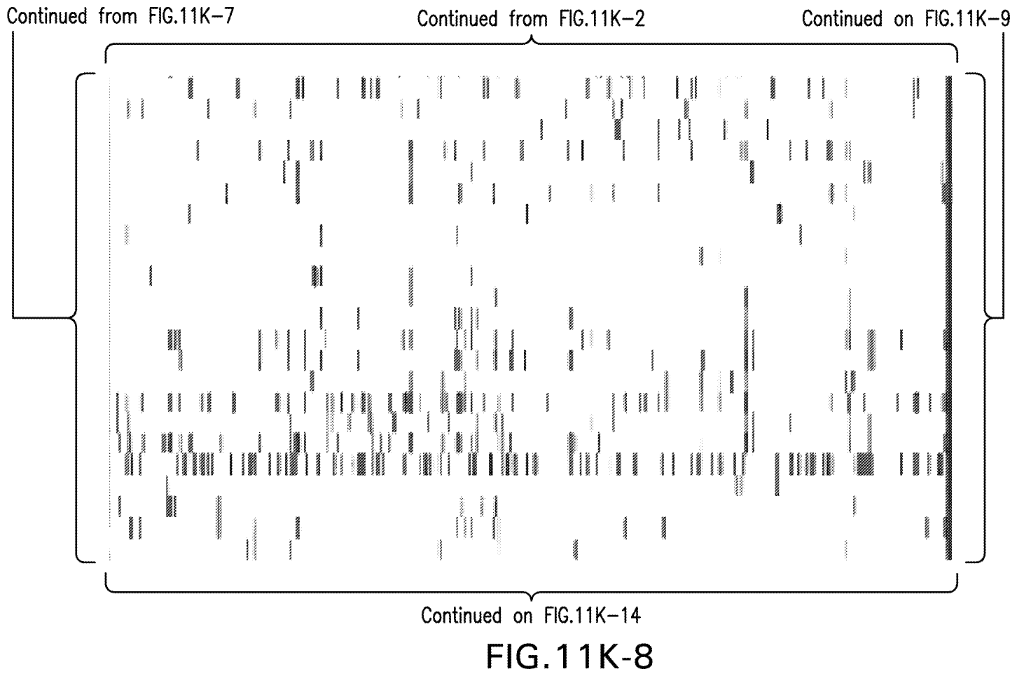

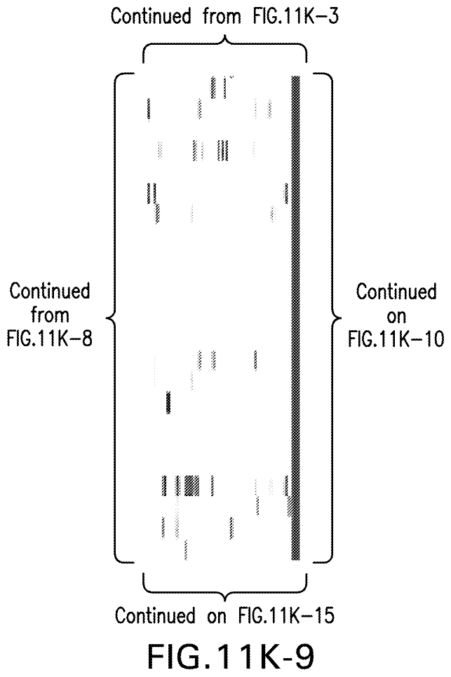

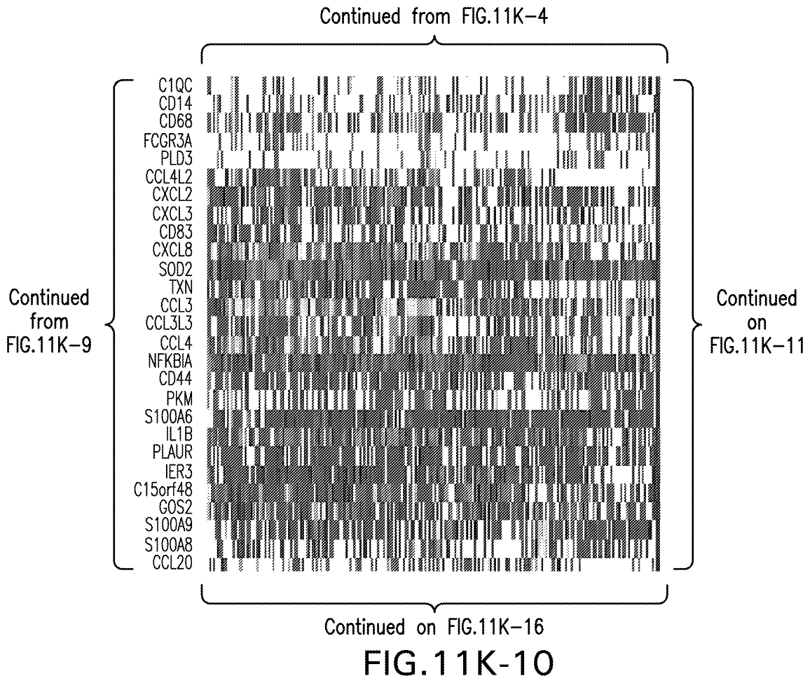

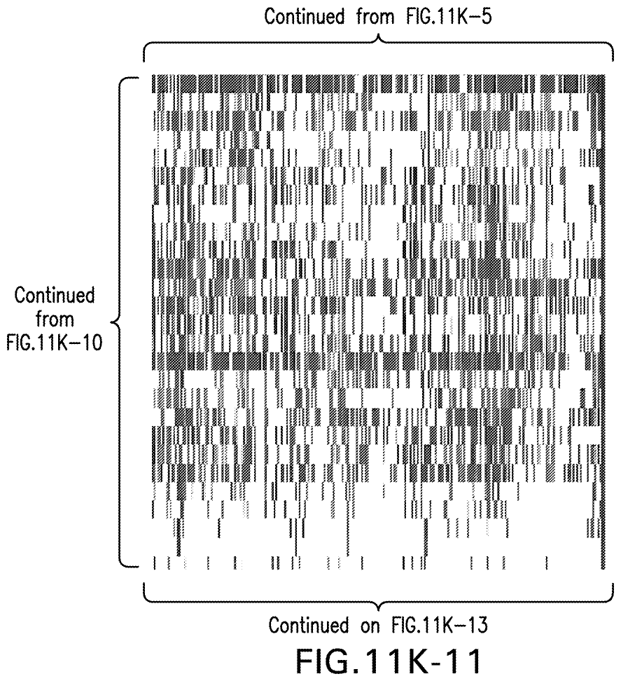

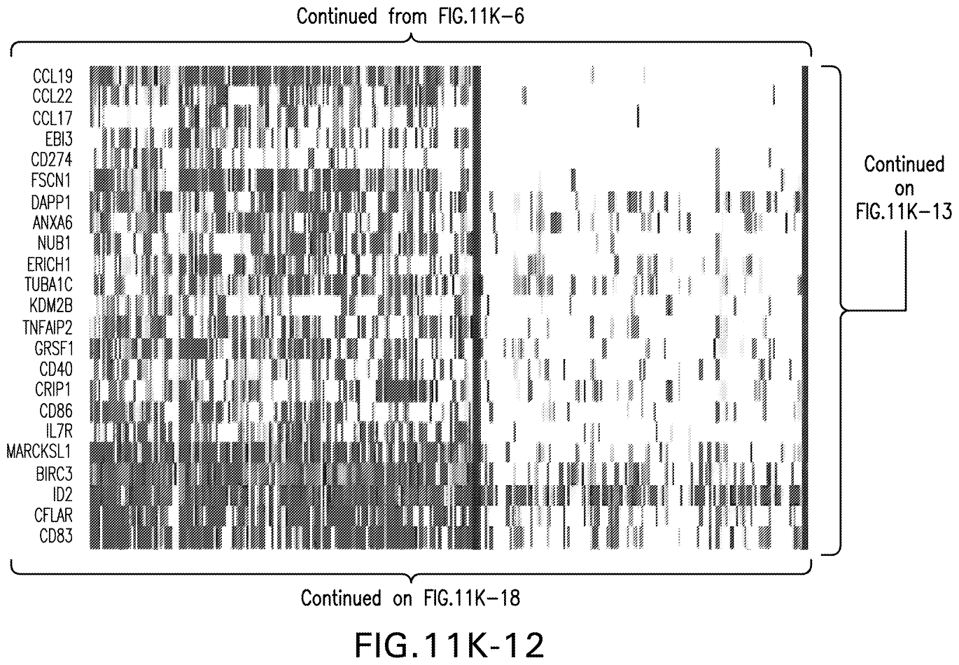

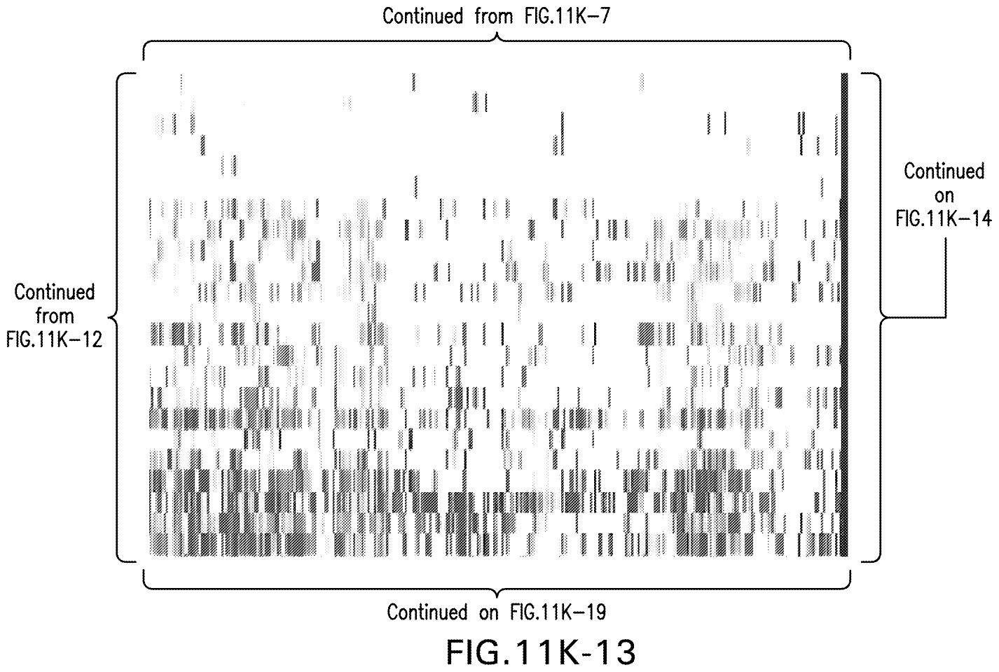

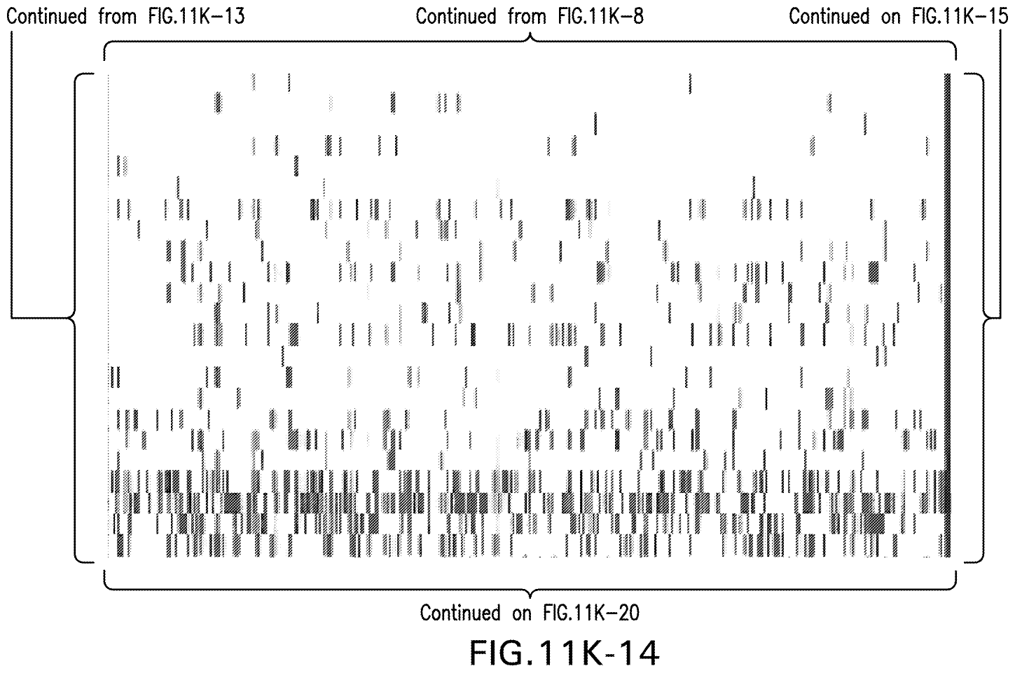

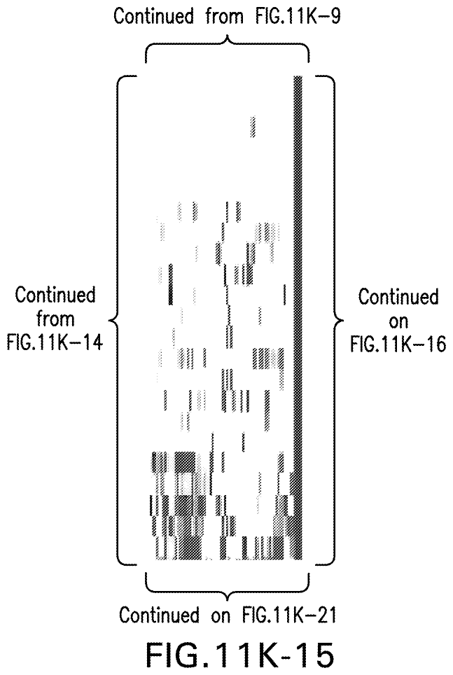

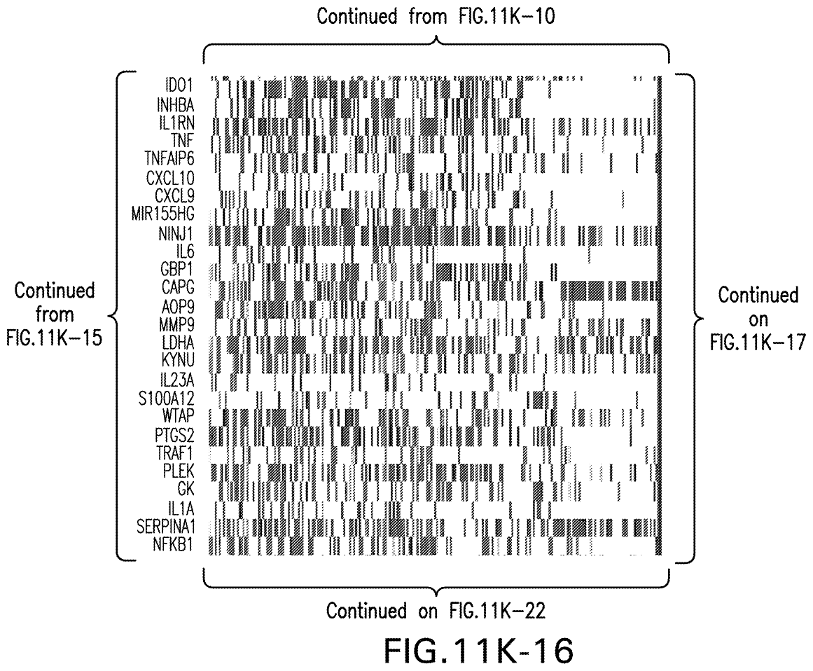

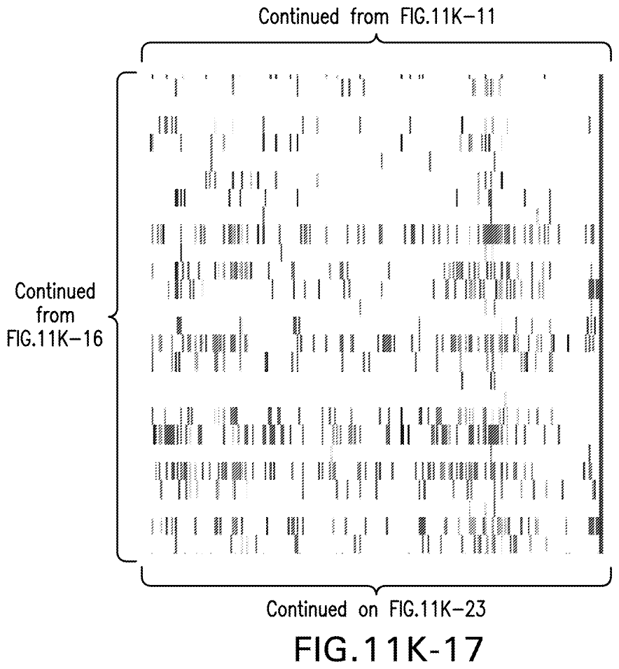

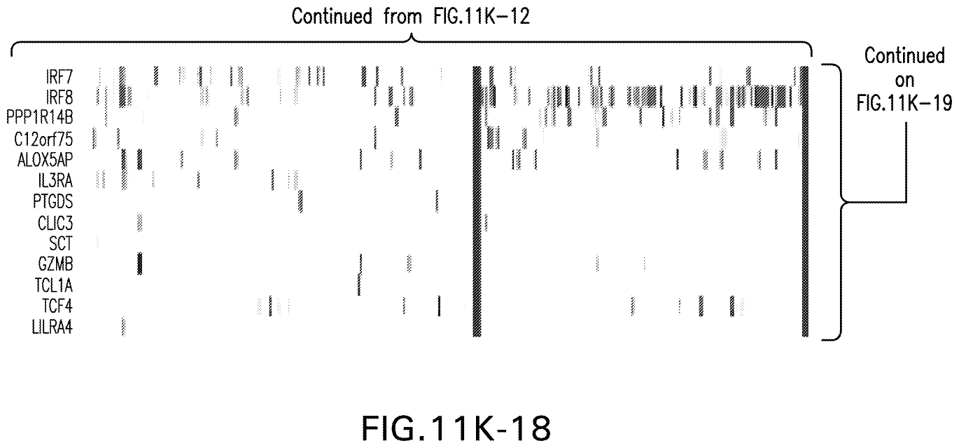

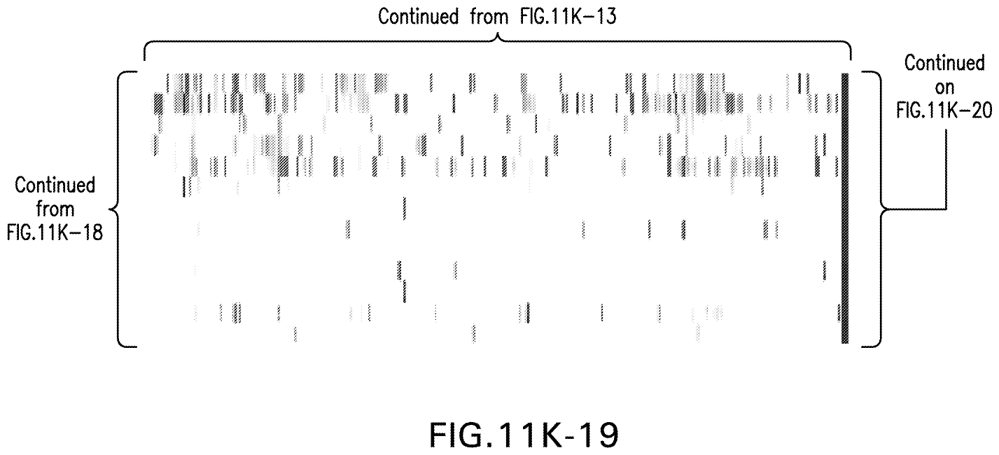

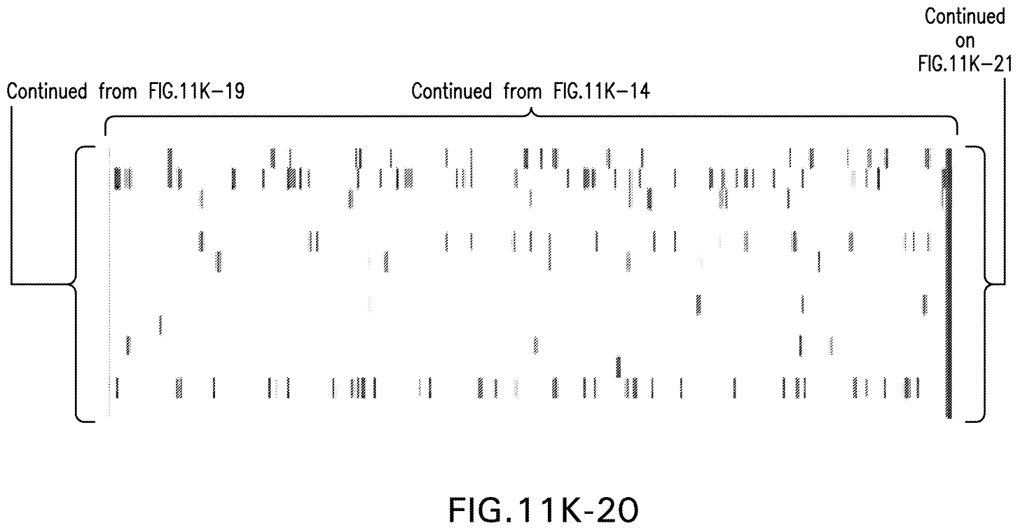

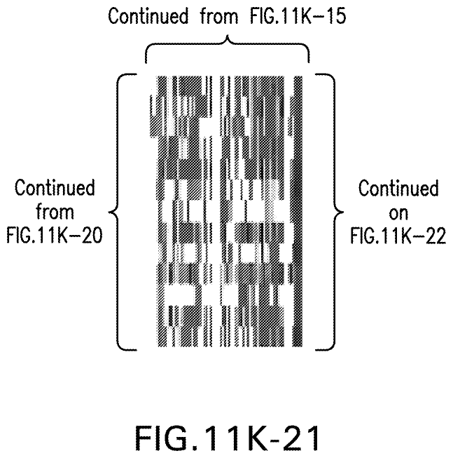

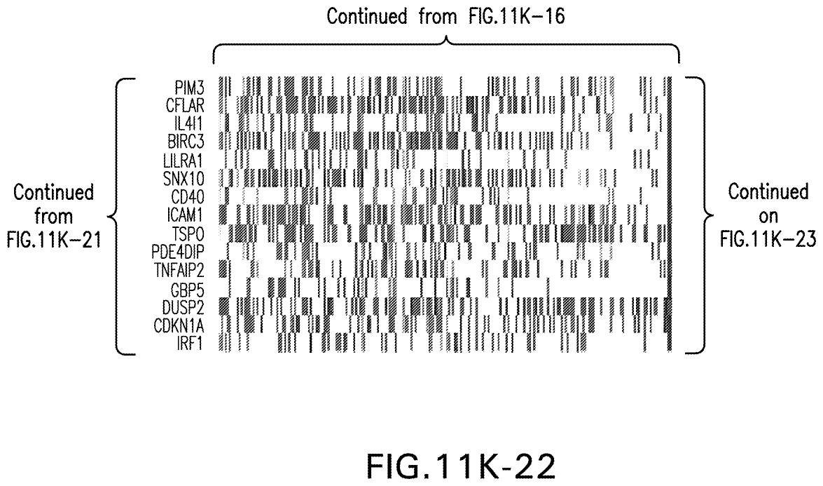

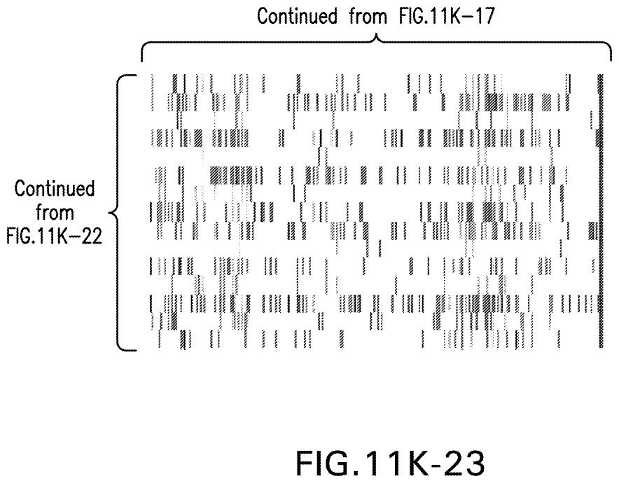

First, single cell RNA-sequencing (“single cell RNA-seq” or “scRNA-seq”) data from CD patients were analyses to confirm that the cells were sufficiently different to be distinguished by RNA-seq based gene expression profile at the single cell level. The data for the single cell RNA-seq analyses along with the cell type and cell subtype annotations were publically available and obtained from Martin J. et al., Cell 178, 1493-1508 (September 2019). As shown in FIGS. 11A and 11B, using t-distributed stochastic neighbourhood embedding (t-SNE) to plot the gene expression profiles of the various cell types in a 2-dimensional space, coarse classes of cells such as B cells, epithelial cells, innate lymphoid cells (ILCs) cells, myloid cells, plasma cells, stromal cells, and T cells were distinguishable (FIG. 11B), and further fine classes or subclasses such as those listed in FIG. 11A were also distinguishable. The various classes and subclasses of cells can also be identified by hierarchical clustering analyses, as shown in FIG. 11C. Thus the RNA-seq data provides sufficient difference to distinguish various cell types and cell sub-types based on gene expression profile.

Next, single cell RNA seq data were analysed for cell type determination. Briefly, single cell RNA seq data from ileum tissue of CD patients were annotated as gene expression based on normalized RNA sequencing reads using expression score (0-6, with 0 being the lowest and 6 being the highest, normalized so that the total reads are the same across the cells). The cell type of each single cell was then annotated using the gene expression profile of pre-annotated human intestinal tract cell as reference (Elmentaite R. et al., Nature 597: 250-255 (Sep. 9, 2021)), and confirmed for known cell type markers for accuracy. As shown in FIGS. 11D and 11E, B cells, Epithelial cells, ILC cells, mast cells, mononuclear phagocyte (MNPs), plasmacytoid dendritic cells (pDCs), plasma cells, stromal cells, and T cells were identified and annotated, and confirmed with the expected cell type markers.

Having established that the various cell types can be correctly identified and annotated based on reference cell gene expression profile and known markers, the disclosure analyzed whether the gene expression profiles of the cell types are sufficiently different to be distinguished with computation algorithms, which help the deconvolution algorithms. Accordingly, unsupervised analyses of differential expressed genes (DEG) were performed to see how different the single cell gene expression profiles were between the different cell types and how consistent they were within the same cell type. The top 10 DEGs for each cell type were shown in FIG. 11F. FIG. 11F demonstrated that the cells within each cell type were consistent in their gene expression profiles, the cells across the cell types had distinguishable gene expression profiles, and the genes that distinguish each cell types are consistent with the known cell type gene markers.

Analyses similar to the previous 2 paragraphs were also performed with respect to sub-cell types within the epithelial cells. As shown in FIGS. 11G and 11H, BEST+ epithelial cells, colonocytes, enteroendocrine cells (EEC), enterocytes, goblet cells, microfold cells, Paneth cells, stem cells, transit-amplifying (TA) progenitor cells, and Tuft cells were identified, annotated, and confirmed with the expression of expected cell type markers (cell subtypes indicated in the top and corresponding markers for each cell subtype indicated in the left column). The unsupervised analyses of top 10 DEG further confirmed that the sub-cell types within the epithelial cells expressed the known markers for the corresponding sub-cell types (data not shown).

For sub-cell types within the stromal cells, Martin J. et al., Cell 178, 1493-1508 (September 2019) provided such sub-cell type annotations. Accordingly, the annotated sub-cell types within the stromal cells, including ACKR1+ endothelial cells, activated fibroblasts, CD36+ endothelial cells, fibroblasts, glial cells, lymphatics, myofibroblasts, pericytes, and smooth muscle cells, were analysed for gene expression based on normalized RNA sequencing reads using expression score (0-6, with 0 being the lowest and 6 being the highest, normalized so that the total reads are the same across the cells), and compared with known gene expression markers for confirmation. As shown in FIG. 11I, each sub-cell type of the stromal cells expressed the expected cell markers. The unsupervised analyses of top 10 DEG further confirmed that the sub-cell types within the stromal cells expressed the known markers for the corresponding sub-cell types (data not shown).

Similarly, for sub-cell types within the T-cells, Martin J. et al., Cell 178, 1493-1508 (September 2019) provided such sub-cell type annotations. Accordingly, the annotated sub-cell types within the T-cells, including cytotoxic T cells, highly activated T cells, ILC1, ILC3, naïve/CM T cells, natural killer (NK) cells, NK T-cells, TFH-like cells, Gamma-delta (γδ) T cells, regulatory T cells, and tissue-resident memory T (Trm) cells, were analysed for gene expression based on normalized RNA sequencing reads using expression score (0-6, with 0 being the lowest and 6 being the highest, normalized so that the total reads are the same across the cells), and confirmed for expression of gene expression markers expected for the corresponding cell type (data not shown). The unsupervised analyses of top 10 DEG further confirmed that the sub-cell types within the T-cells expressed the known markers for the corresponding sub-cell types (data not shown). Additionally, as shown in FIG. 11J, each sub-cell type of the T-cells expressed the expected cytokines at a level and percentage consistent with the corresponding T cell subtype, thereby further confirming the annotation of the sub-cell types within the T-cells.

For sub-cell types within the myloid cells, Martin J. et al., Cell 178, 1493-1508 (September 2019) provided such sub-cell type annotations. Accordingly, the annotated sub-cell types within the myloid cells, including activated dendritic cells (DCs), DC1, DC2, monocyte-derived dendritic cells (moDC), plasmacytoid dendritic cells (pDCs), inflammatory macrophages, and resident macrophages, were analysed for gene expression based on normalized RNA sequencing reads using expression score (0-6, with 0 being the lowest and 6 being the highest, normalized so that the total reads are the same across the cells), and compared with known gene expression markers for confirmation. As shown in FIG. 11K, each sub-cell type of the myloid cells expressed the expected cell markers. The unsupervised analyses of top 10 DEG further confirmed that the sub-cell types within the myloid cells expressed the known markers for the corresponding sub-cell types (data not shown).

Accordingly, starting from publically available data from Martin J. et al., Cell 178, 1493-1508 (September 2019), single cell RNA-seq-based gene expression profiles are provided herein for various cell types and sub-cell types related to IBD diseased tissues, which can be the references for deconvoluting the bulk RNA sequencing data of diseased tissues from IBD patients as further described below.

### 7.20.2 Subpart 2. Validation of Methodology for Deconvoluting Bulk RNA Sequencing Data into Cell Types and Sub-Cell Types

Having established the scRNA-seq-based gene expression profiles for various cell types and sub-cell types related to IBD diseased tissues, deconvolution methodology based on Wang X et al., Nature Communications (2019) 10:380, termed MUlti-Subject SIngle Cell or MuSiC (incorporated herein in its entirety by reference), was performed to confirm the suitability of the methodology to IBD patient samples. Briefly, MuSiC deconvolves bulk RNA-seq samples to obtain the proportions of cell types or sub-cell types in each sample. Incorporated in MuSiC is two types of “marker gene consistency”: cross-subject and cross-cell, in which the first is to guard against bias in subject selection, and the second is to guard against bias in cell capture in scRNA-seq. By incorporating both types of consistency, MuSiC allows for scRNA-seq datasets as described in Section 7.20.1 to serve as effective references for independent bulk RNA-seq datasets involving different individuals. Rather than pre-selecting marker genes from scRNA-seq based only on mean expression, MuSiC gives weight to each gene, allowing for the use of a larger set of genes in deconvolution. The weighting scheme prioritizes consistent genes across subjects: up-weighing genes with low cross-subject variance (informative genes) and down-weighing genes with high cross-subject variance (non-informative genes). Such requirement provides for cross-subject consistency needed for transferring cell type-specific gene expression information from one dataset to another. MuSiC also employs a tree-guided procedure that recursively zooms in on closely related cell types. MuSiC groups similar cell types into the same cluster and estimate cluster proportions, then recursively repeat this procedure within each cluster. At each recursion stage, MuSiC only use genes that have low within-cluster variance, which are the cross-cell consistent genes.

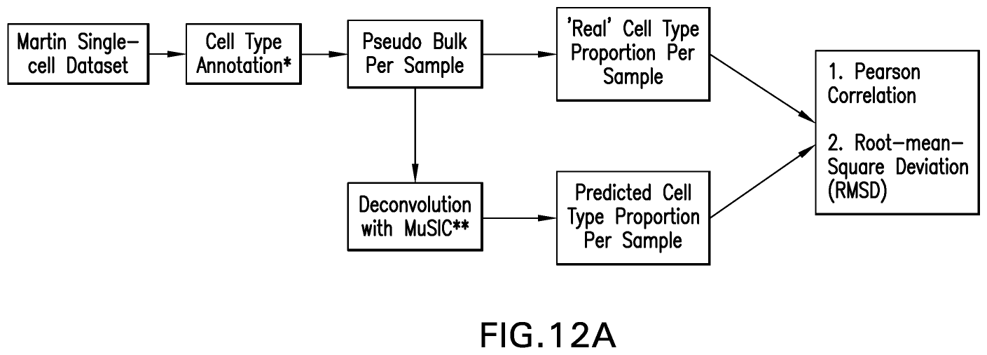

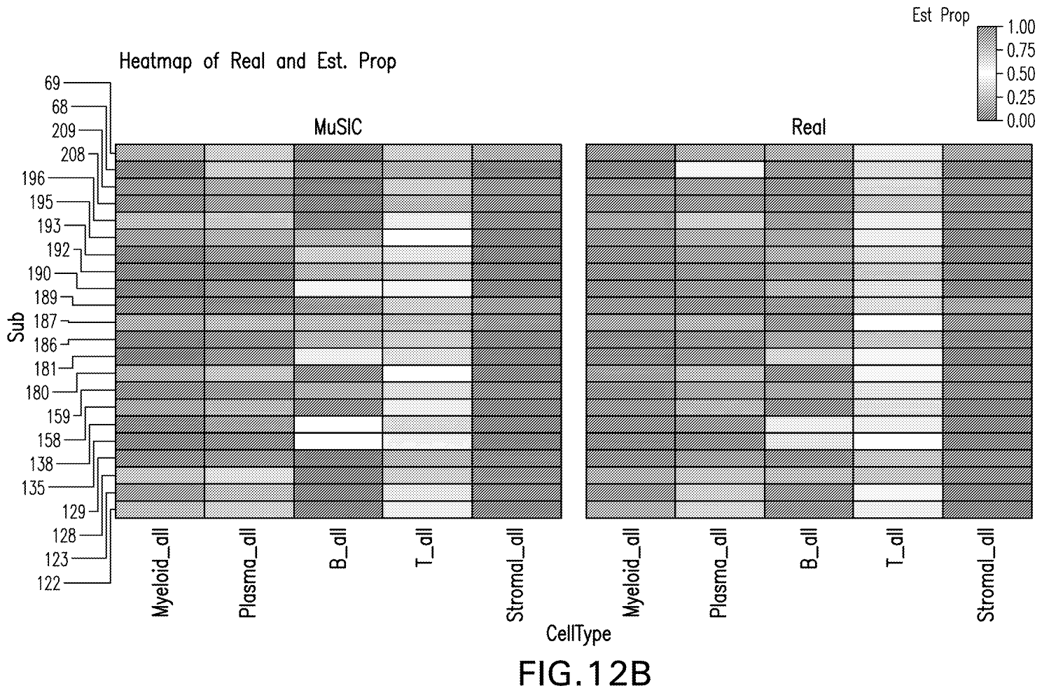

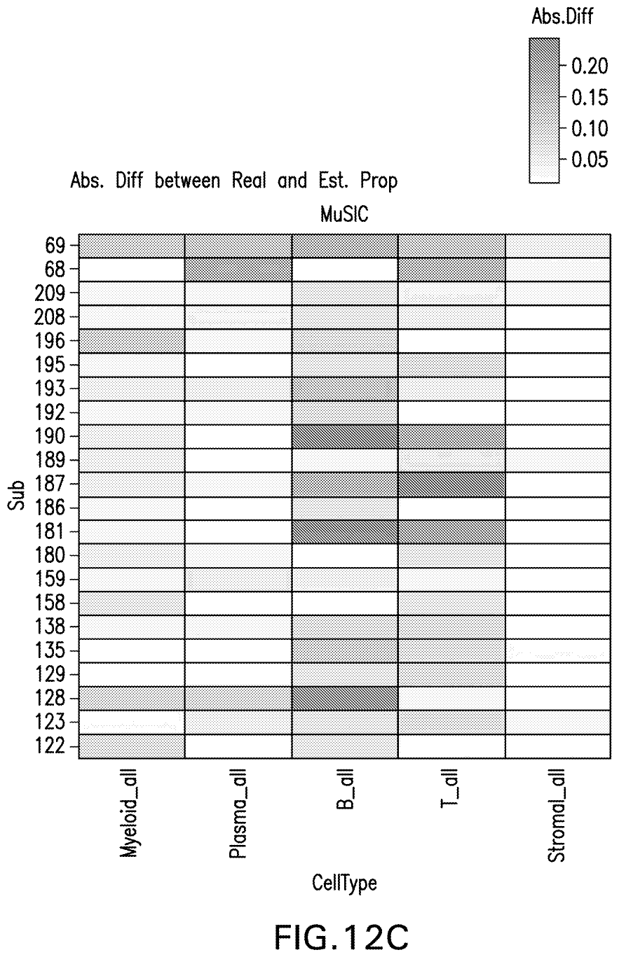

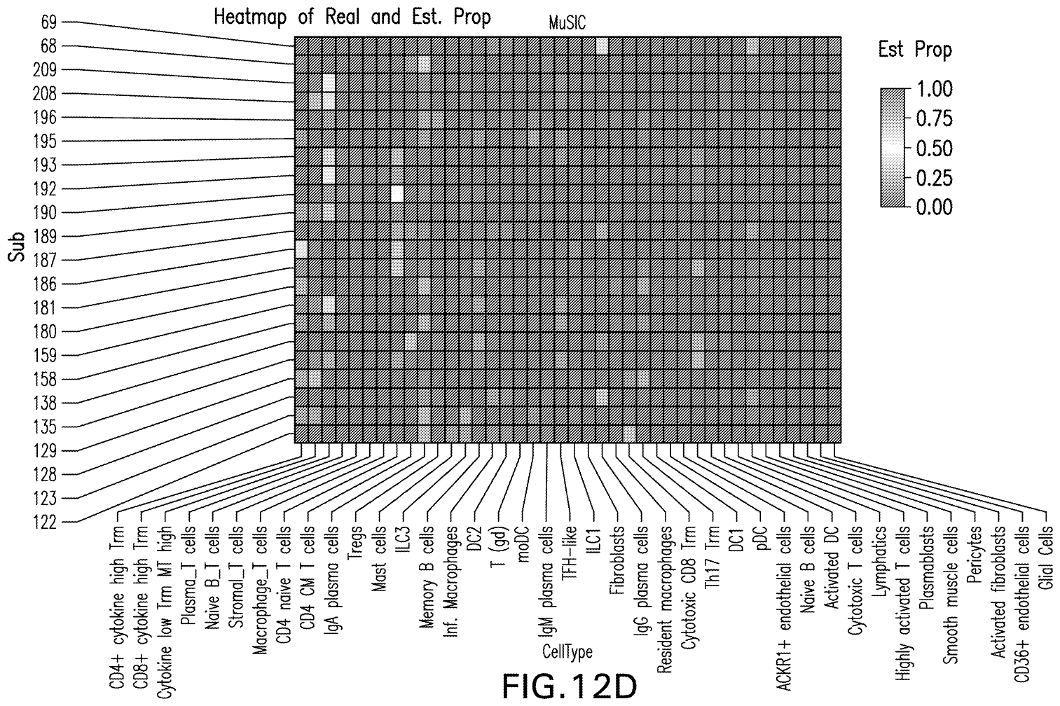

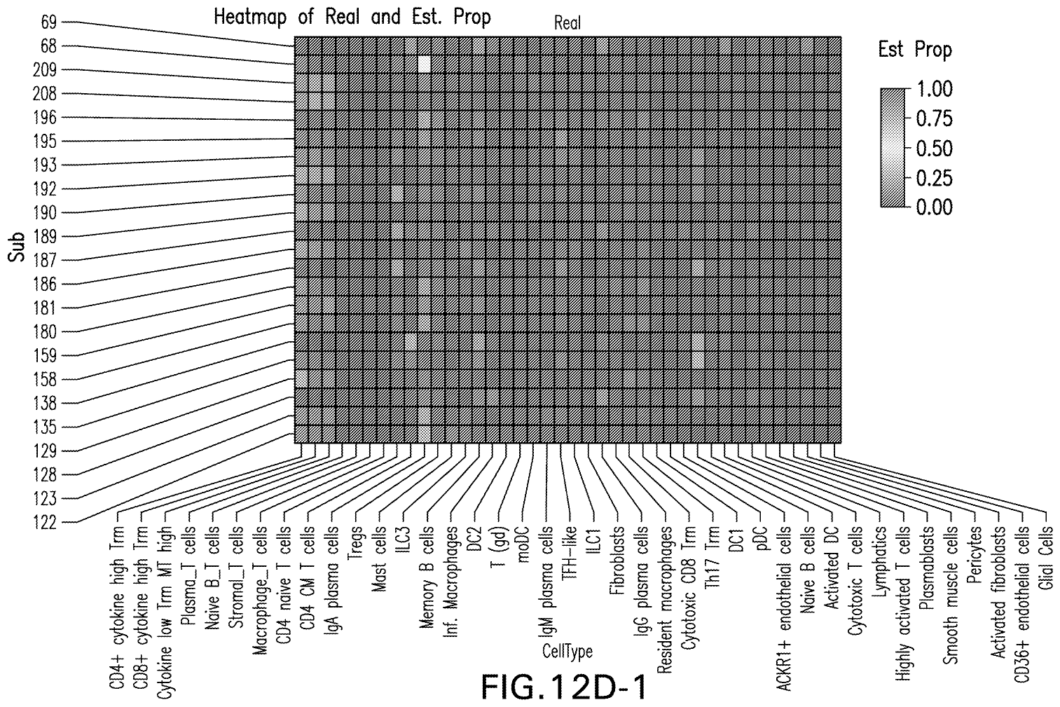

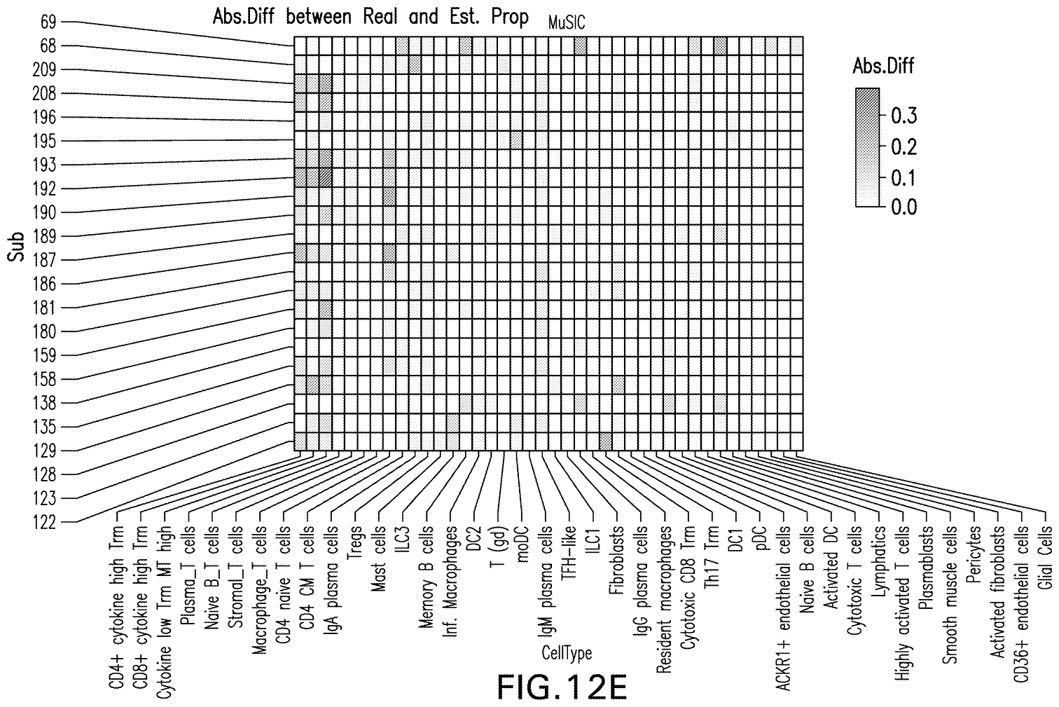

A pseudo-bulk RNA sequencing dataset was first constructed by combining the scRNA-seq data from Martin J. et al., Cell 178, 1493-1508 (September 2019). Such pseudo-bulk RNA sequencing dataset was then deconvolved using MuSiC and the resulting deconvoluted results were compared with the original scRNA-seq data to confirm the accuracy of the deconvolution methodology (FIG. 12A). Two parameters, Pearson correlation and root mean square deviation (RMSD) were calculated to evaluate whether the deconvoluted cell types and their proportions matched the known single cells used to construct the pseudo-bulk sequencing data. As shown in FIGS. 12B and 12C, the cell types and their respective proportions deconvoluted from the pseudo-bulk RNA sequencing data matched well with the single cell RNA-seq data used to construct the pseudo-bulk data, with strong Pearson correlations and low RMSDs (Table 29 and Table 30).

TABLE 29
Correlations and RSMDs between the cell types and their respective
proportions deconvolved from the pseudo-bulk RNA sequencing
data and the single cell RNA-seq data used to construct the
pseudo-bulk data, as calculated for each cell type.

| | Myeloid_all | Plasma_all | B_all | T_all | Stromal_all |
|---|---|---|---|---|---|
| Pearson | 0.903 | 0.953 | 0.812 | 0.894 | 0.937 |
| Correlation | | | | | |
| RSMD | 0.052 | 0.056 | 0.116 | 0.098 | 0.016 |

TABLE 30
Correlations and RSMDs between the cell types and their respective proportions
deconvolved from the pseudo-bulk RNA sequencing data and the single cell RNA-seq
data used to construct the pseudo-bulk data, as calculated for each patient.

| Patients | 69 | 68 | 209 | 208 | 196 | 195 | 193 | 192 | 190 | 189 | 187 |
|---|---|---|---|---|---|---|---|---|---|---|---|
| Pearson | 0.392 | 0.928 | 0.992 | 0.995 | 0.907 | 0.985 | 0.956 | 0.989 | 0.81 | 0.995 | 0.563 |
| Correlation | | | | | | | | | | | |
| RMSD | 0.12 | 0.105 | 0.037 | 0.037 | 0.062 | 0.045 | 0.07 | 0.039 | 0.133 | 0.035 | 0.132 |
| Patients | 186 | 181 | 180 | 159 | 158 | 138 | 135 | 129 | 128 | 123 | 122 |
| Pearson | 0.992 | 0.799 | 0.99 | 0.993 | 0.993 | 0.961 | 0.96 | 0.998 | 0.531 | 0.978 | 0.961 |
| Correlation | | | | | | | | | | | |
| RMSD | 0.034 | 0.139 | 0.032 | 0.033 | 0.036 | 0.052 | 0.062 | 0.046 | 0.119 | 0.051 | 0.045 |

Such pseudo-bulk RNA sequencing data were further deconvoluted for sub-cell types to further validate the accuracy of the MuSiC methodology. As shown in FIG. 12D, the various sub-cell types and their respective proportions deconvolved from the pseudo-bulk RNA sequencing data matched well with the single cell RNA-seq data used to construct the pseudo-bulk data, with low absolute differences observed (FIG. 12E).

Accordingly, the disclosure provides a validated methodology for deconvoluting bulk RNA sequence data into cell types, sub-cell types, and their respective proportions.

### 7.20.3 Subpart 3. Cell Types and Sub-Cell Types in IBD Patients Based on Deconvolution from Bulk RNA Sequencing Data

The reference scRNA-seq-based gene expression profiles for various cell types and sub-cell types related to IBD diseased tissues and deconvolution methodology based on MuSiC algorithm were then applied to bulk RNA sequencing data from IBD patients to determine the cell types and sub-cell types and their respective proportion. Four bulk RNA sequencing datasets were deconvoluted, including Mount Sinai GSE83687 RNAseq Dataset (available from NCBI GEO (gene expression omnibus) by searching with GSE83687, ncbi.nlm.nih.gov/geo/query/acc.cgi?acc=GSE83687), Lloyd Price GSE111889 RNAseq Dataset (available from NCBI GEO by searching with GSE111889, ncbi.nlm.nih.gov/geo/query/acc.cgi?acc=GSE111889), bulk RNA sequencing dataset from a cohort of 119 UC patients at Cedars Sinai Medical Center (referred to as UC119), and bulk RNA sequencing dataset from a cohort of 100 CD patients at Cedars Sinai Medical Center (referred to as CD100).

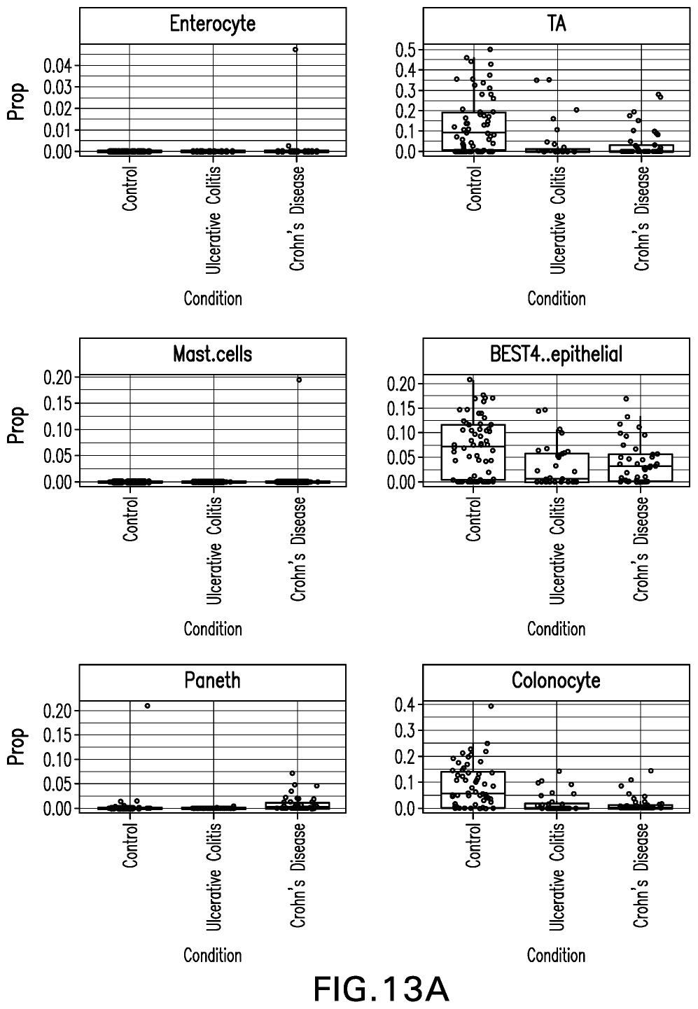

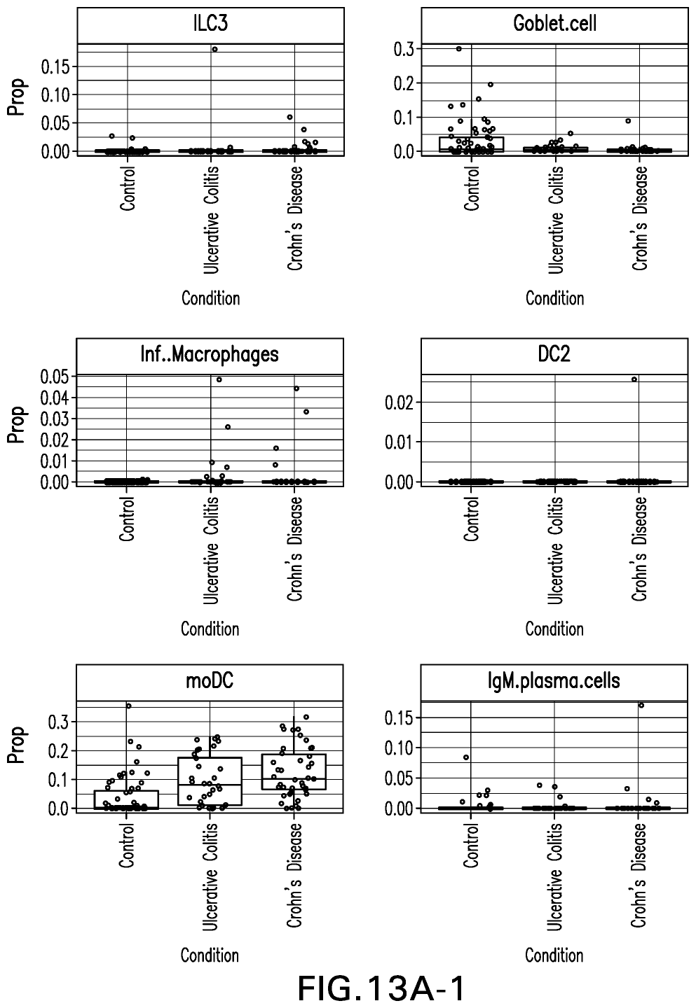

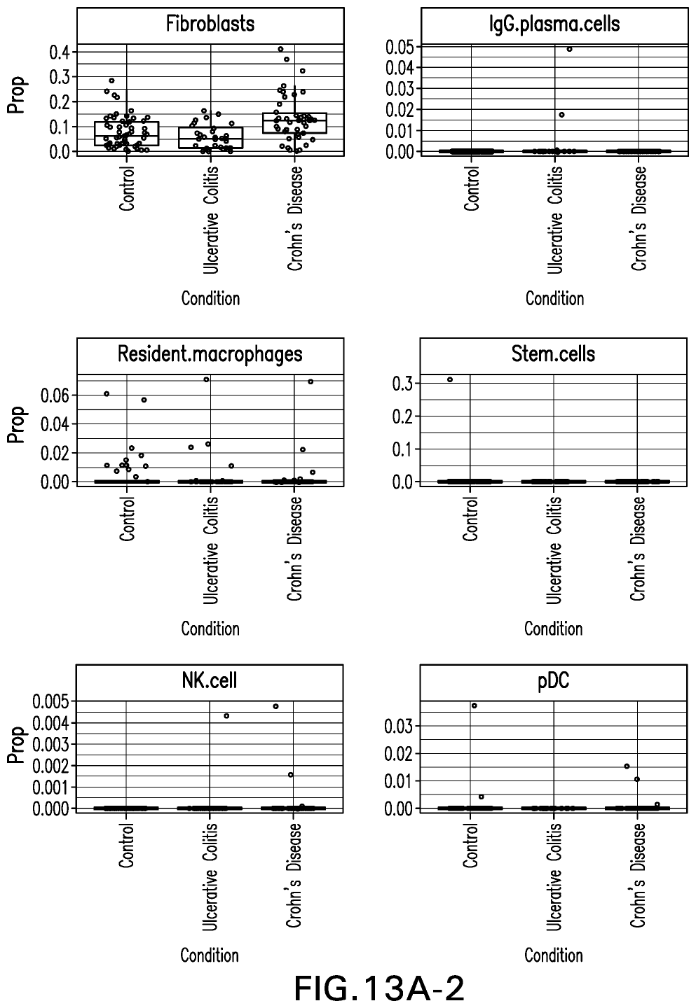

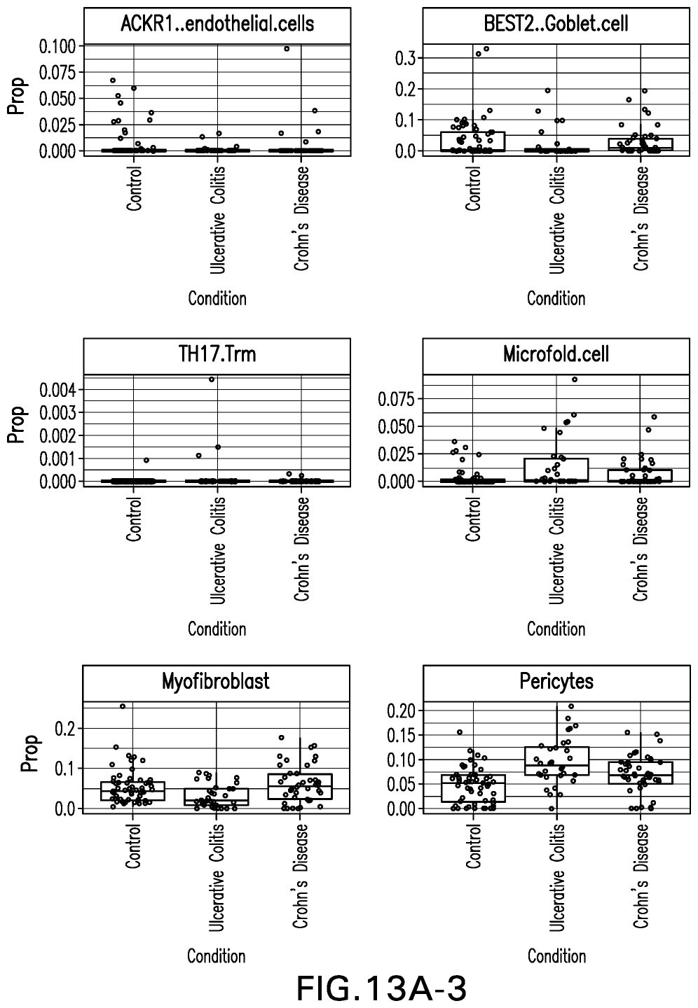

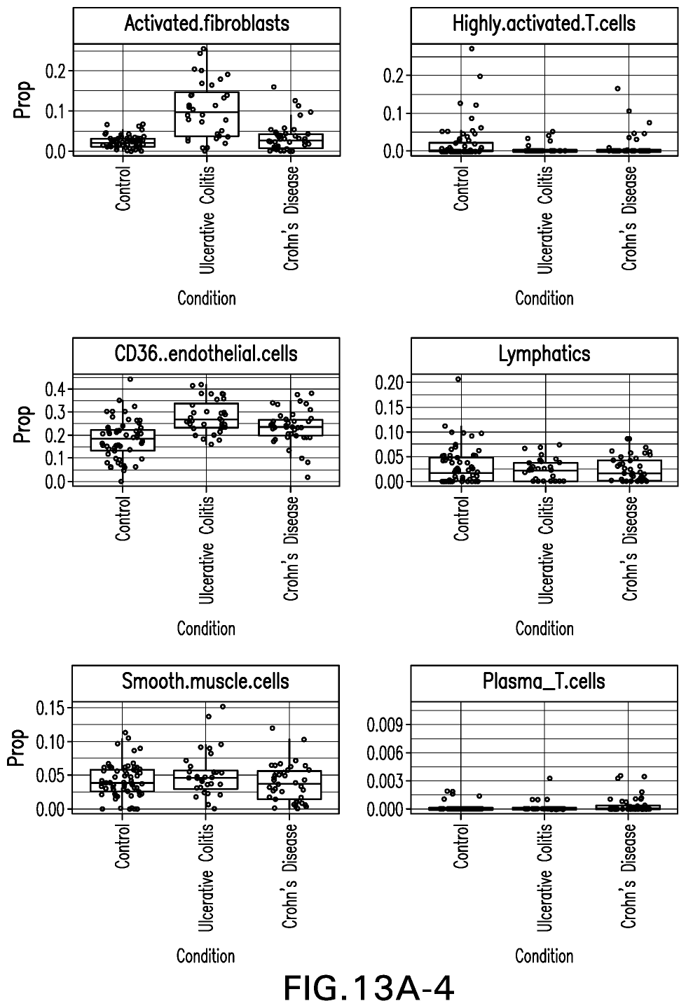

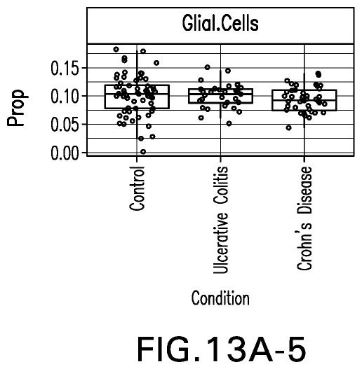

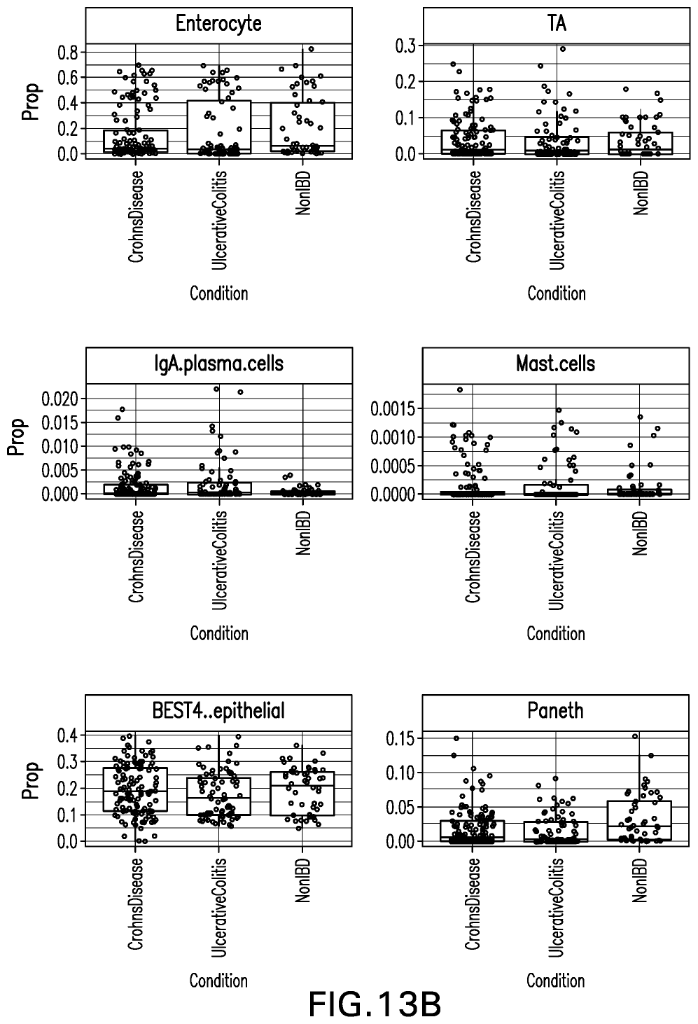

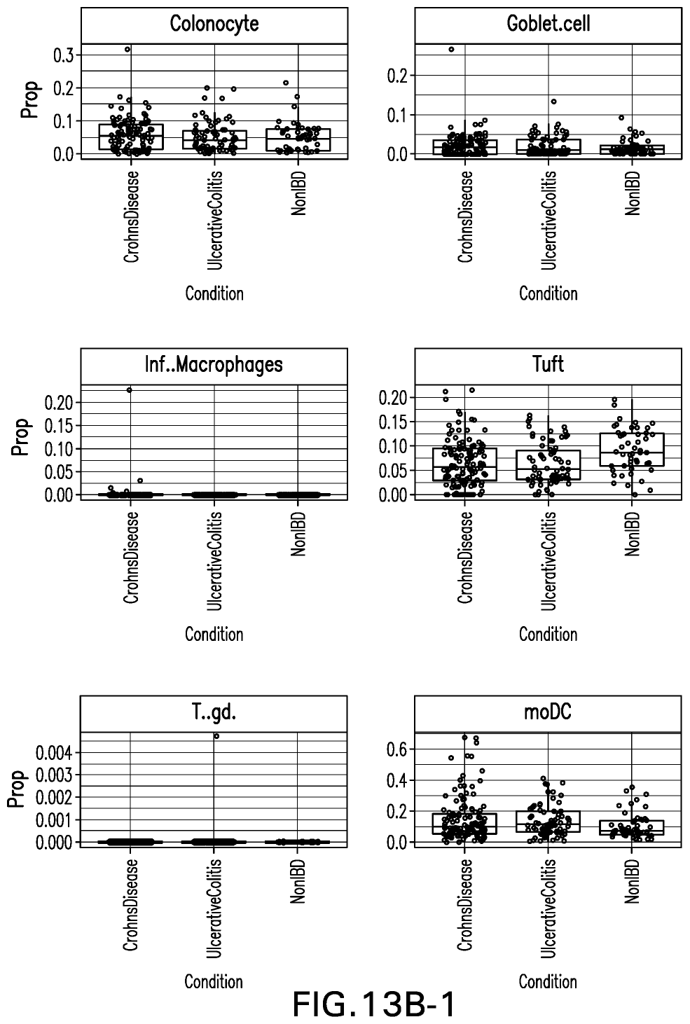

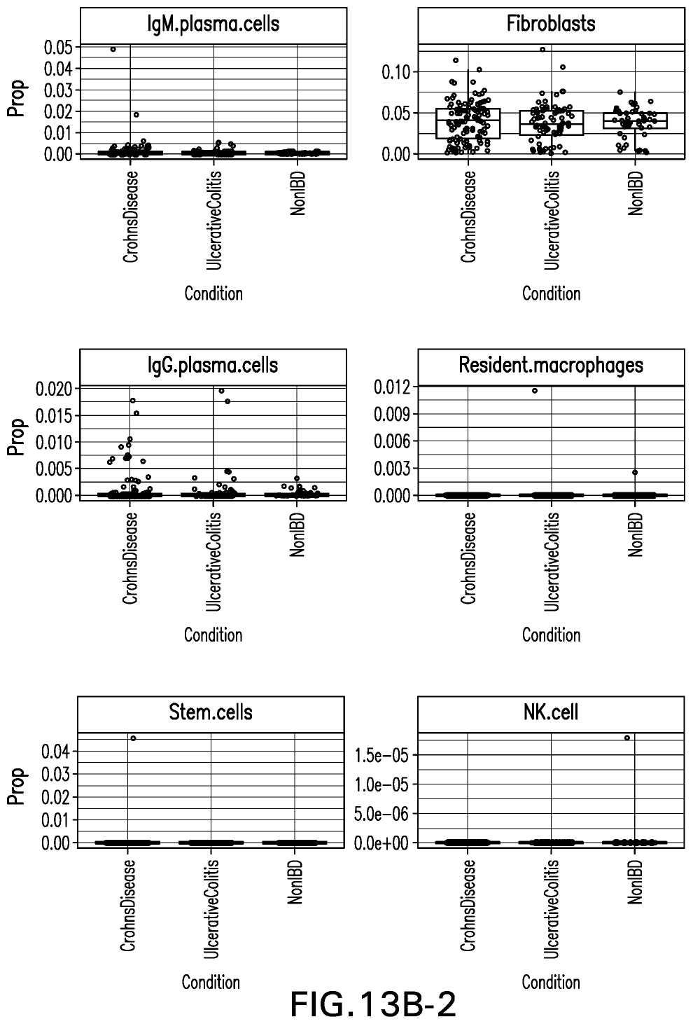

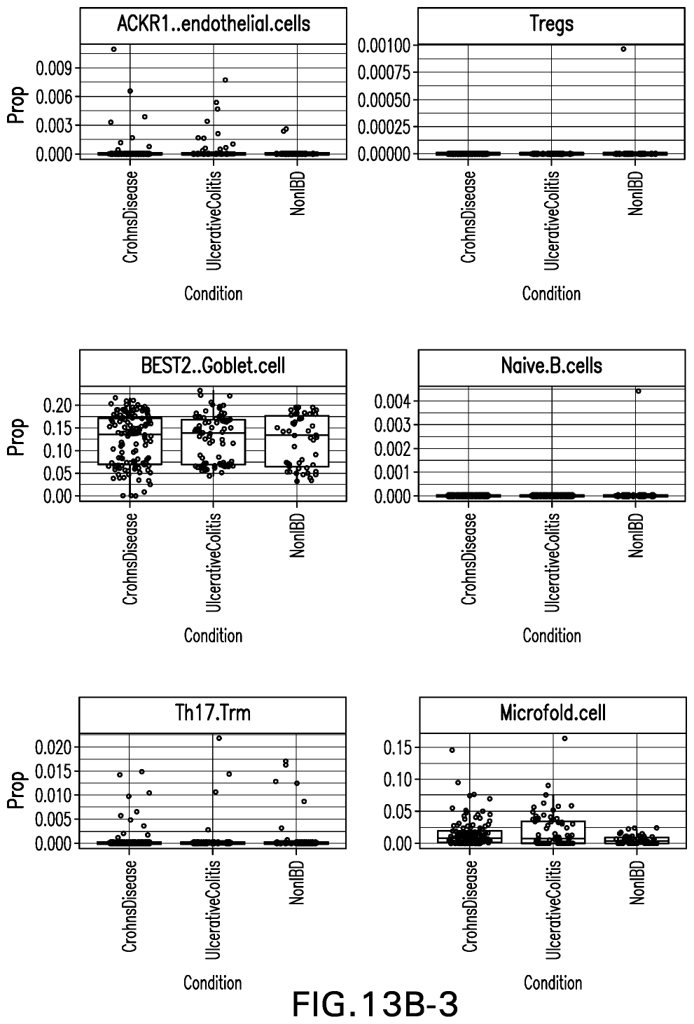

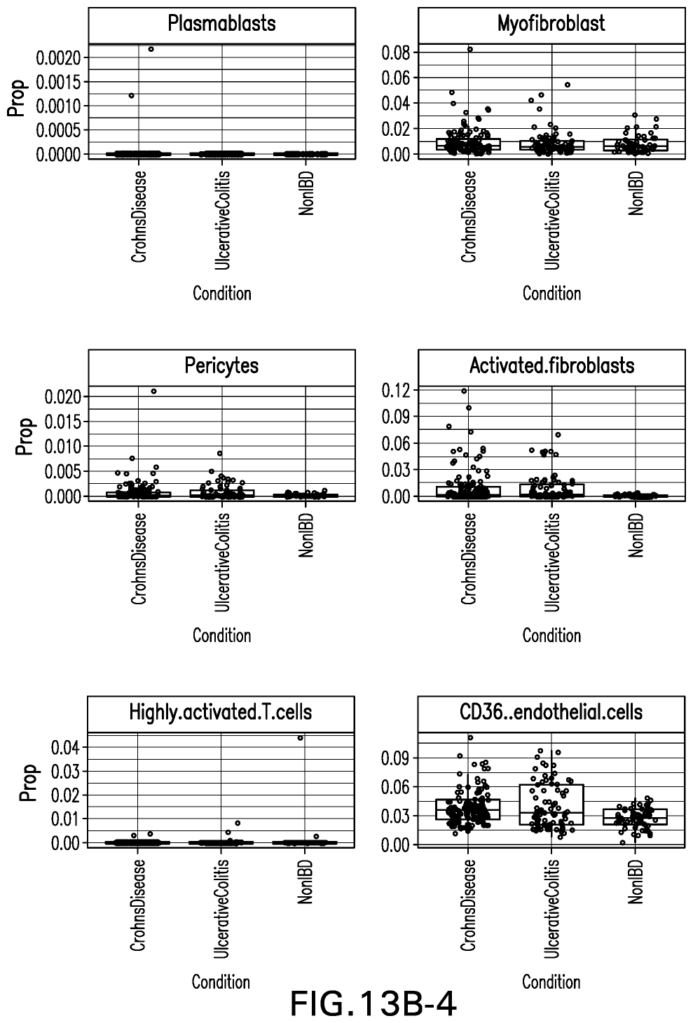

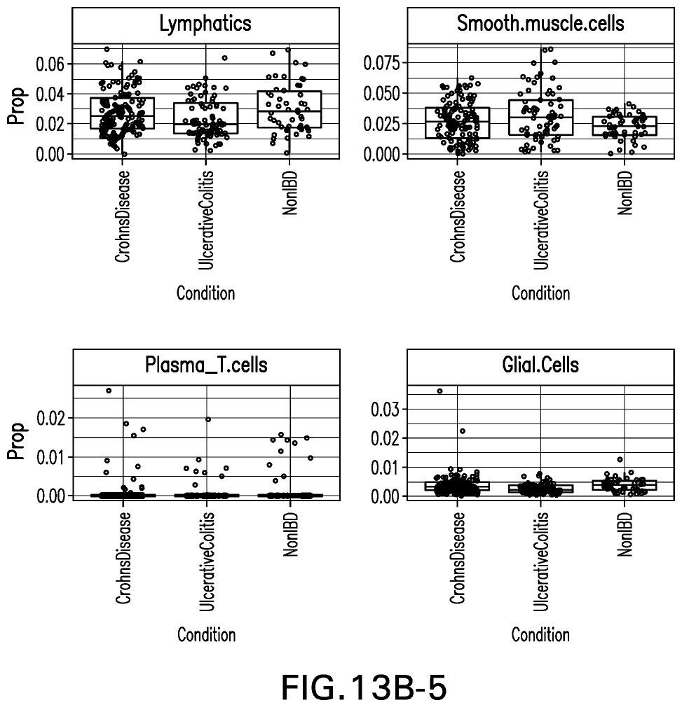

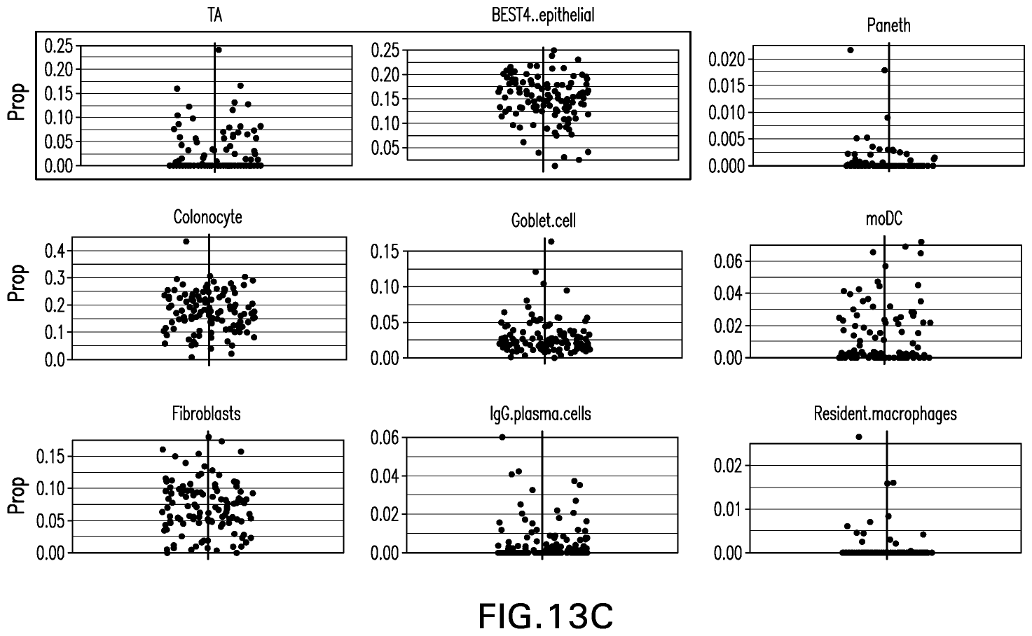

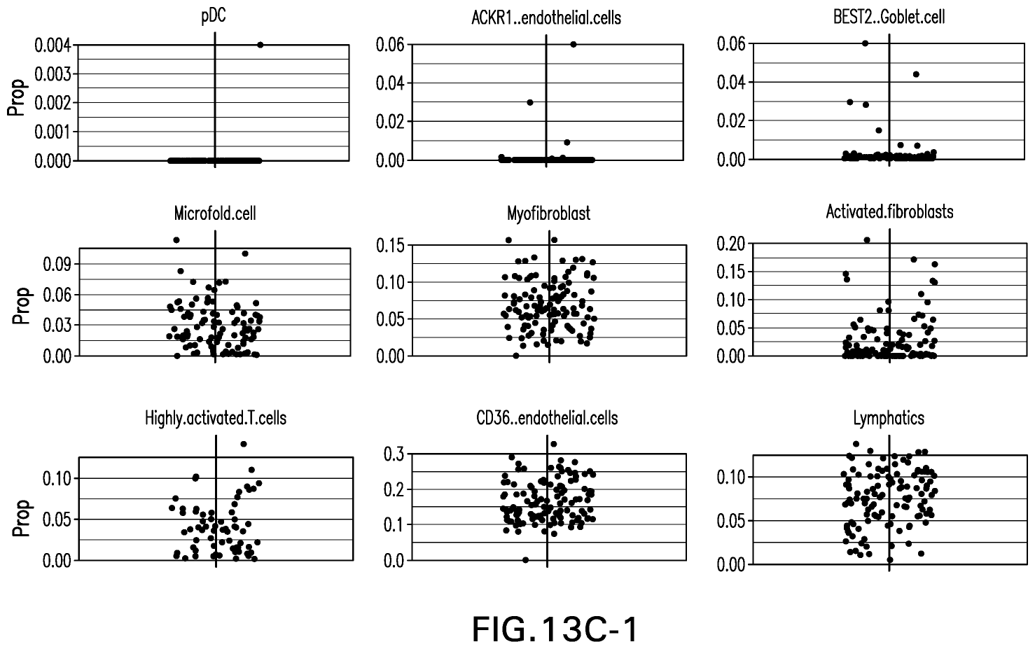

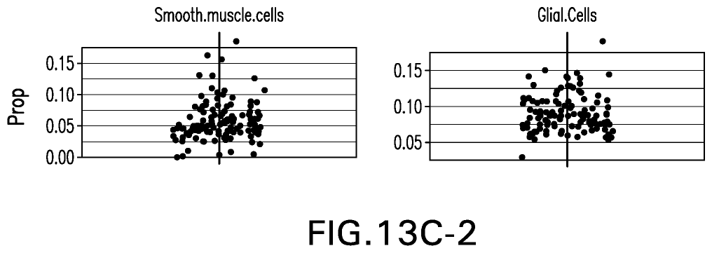

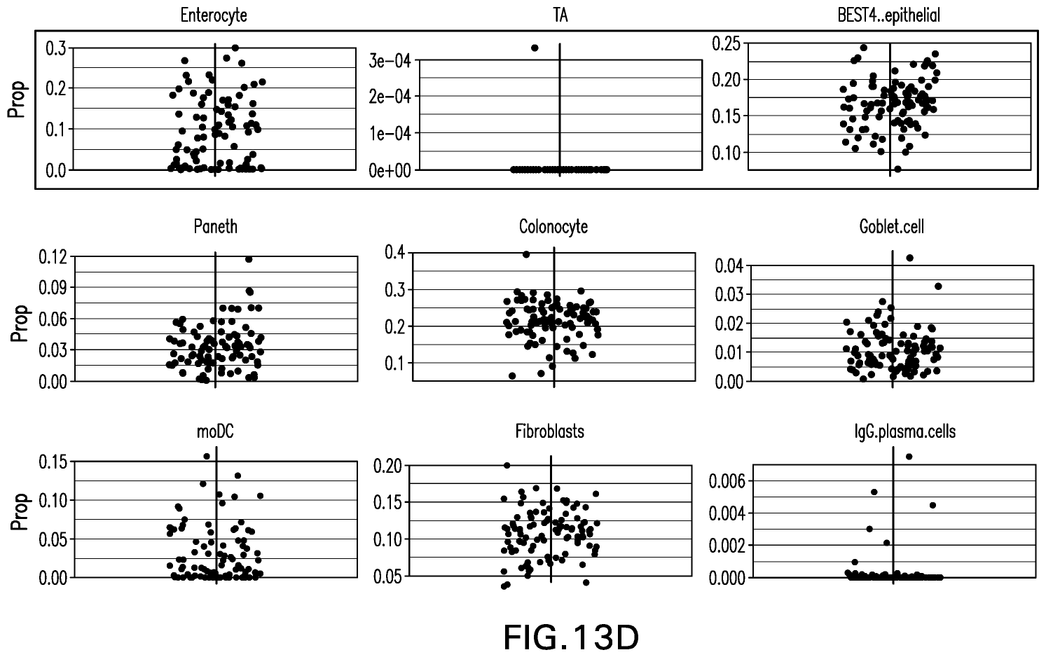

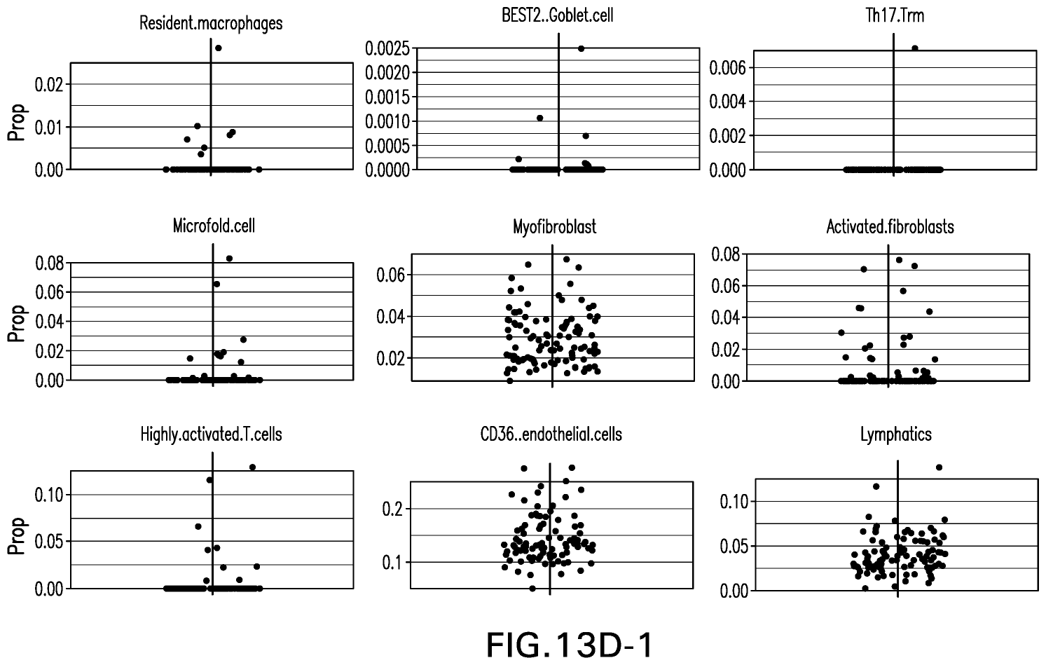

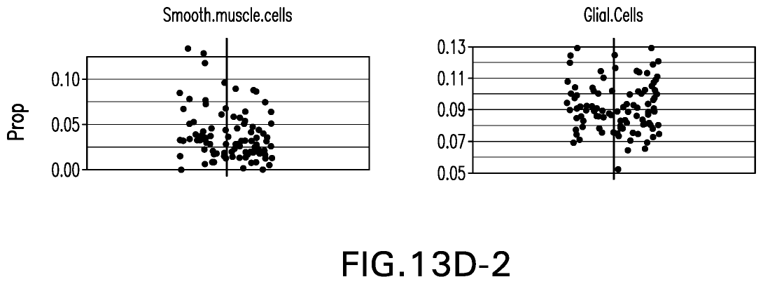

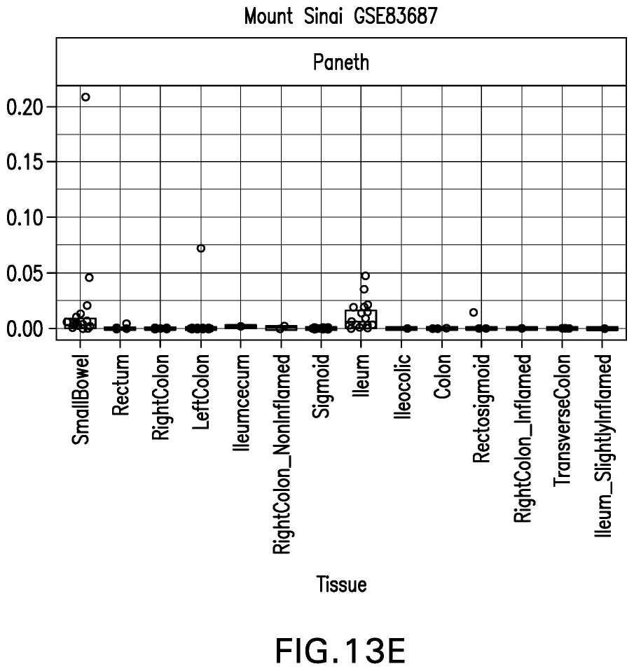

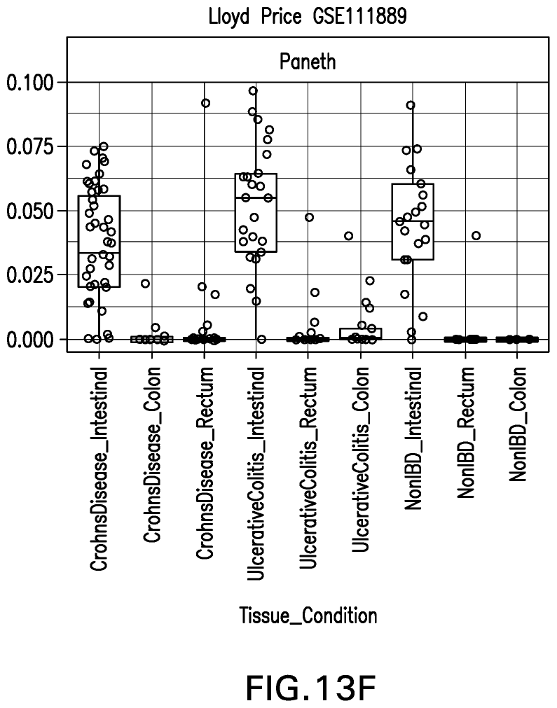

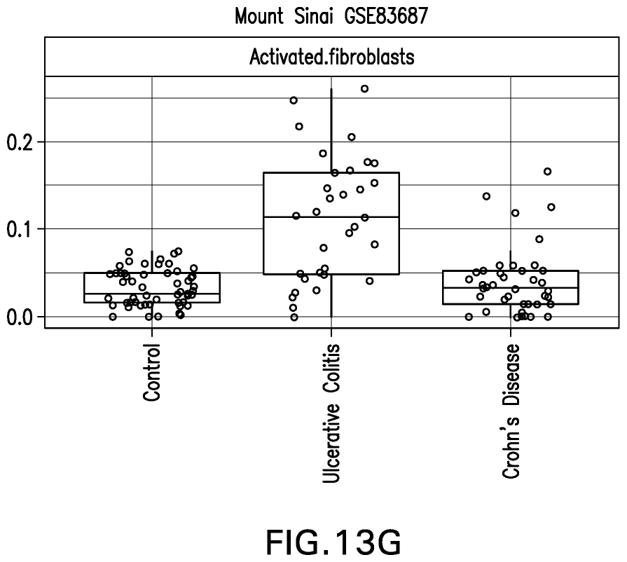

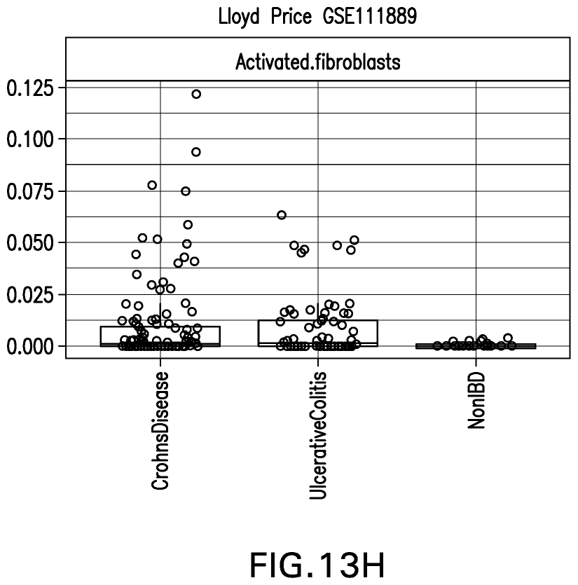

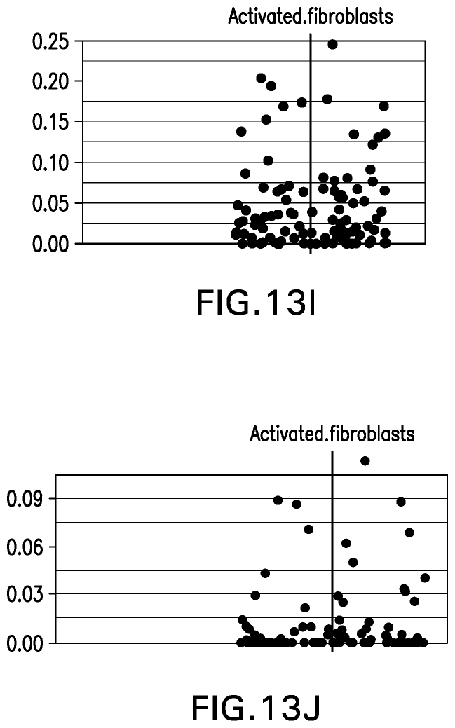

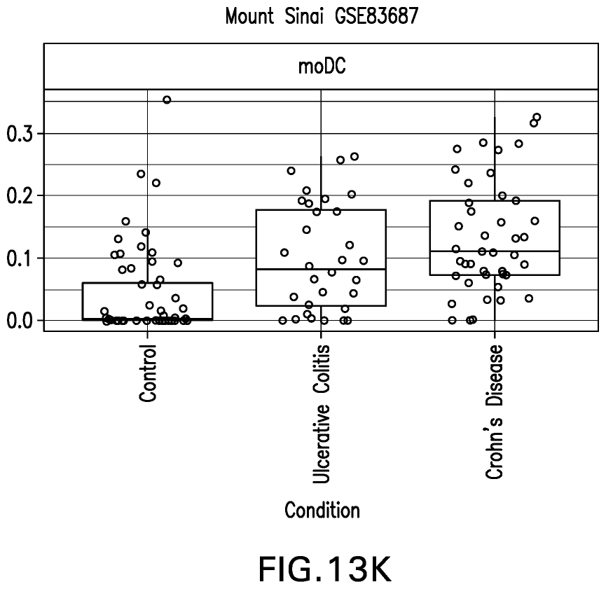

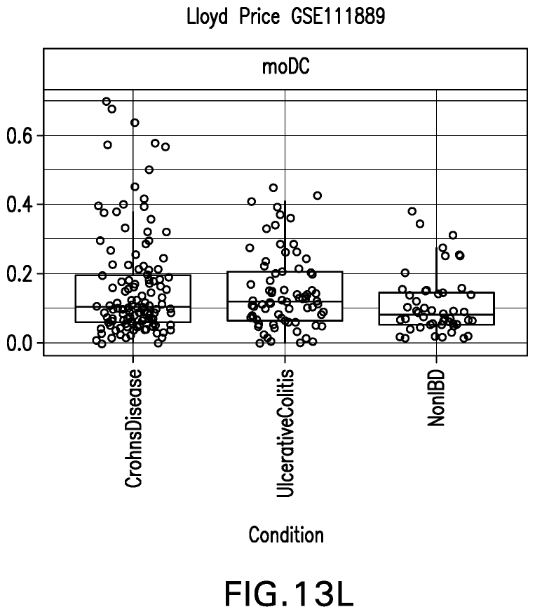

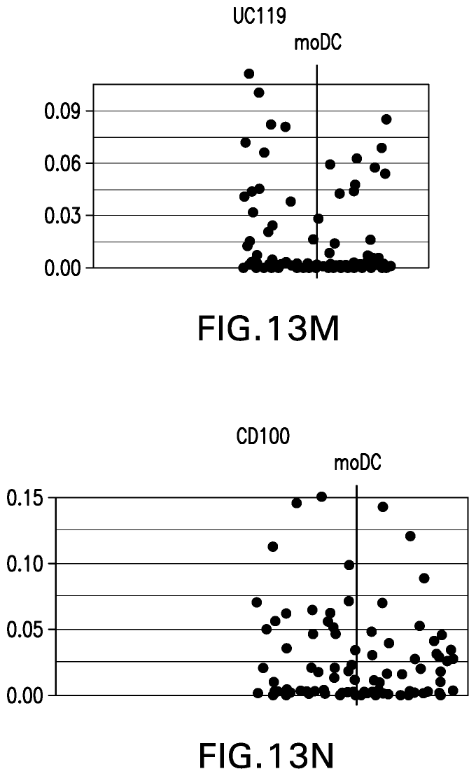

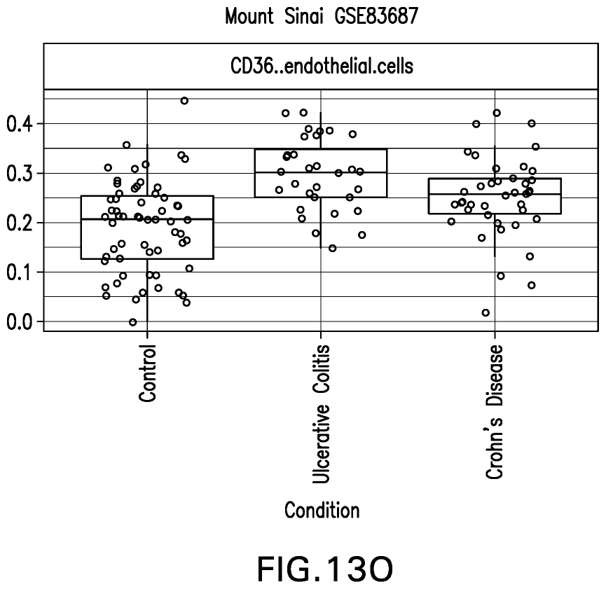

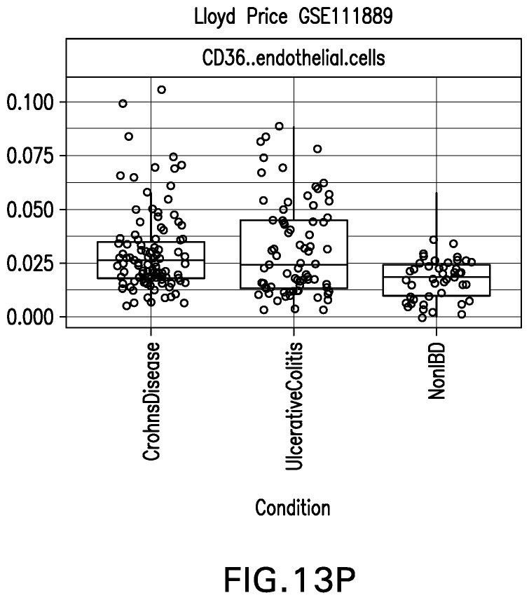

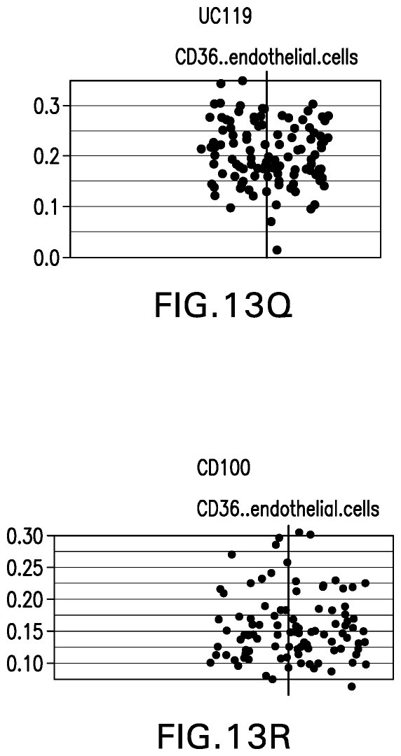

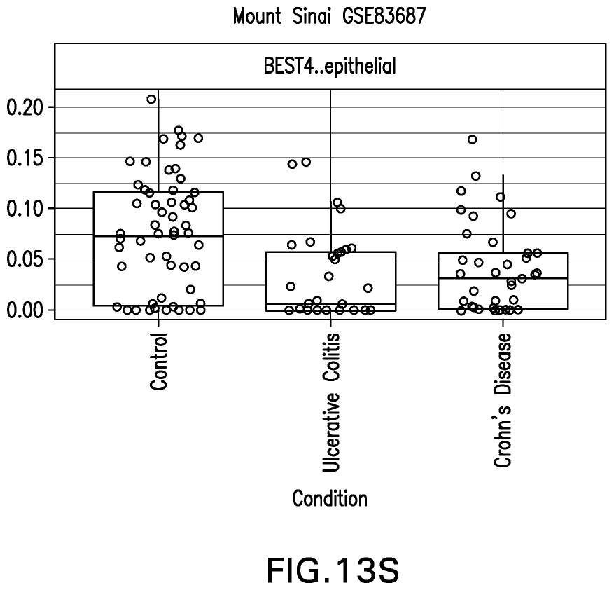

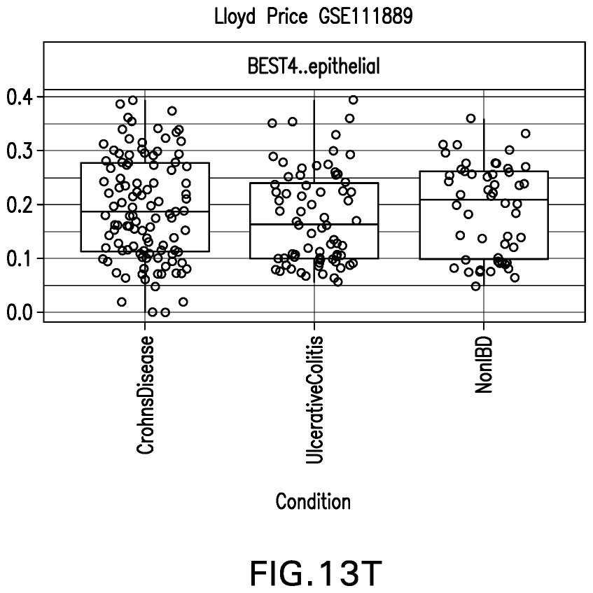

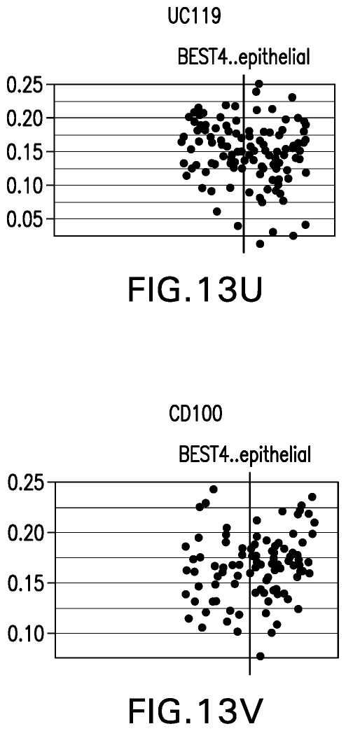

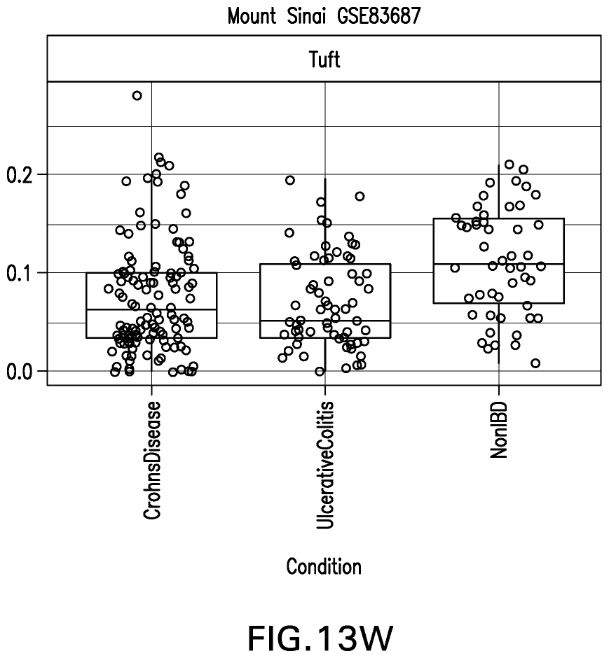

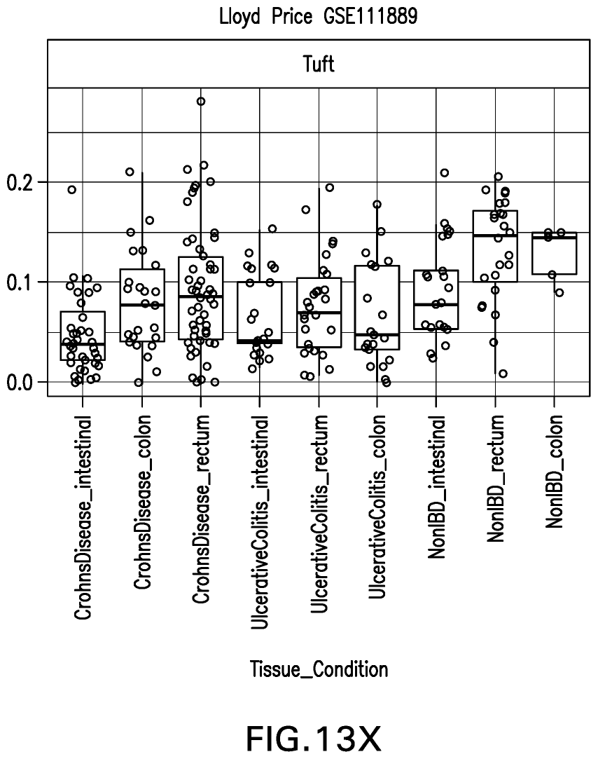

As shown in FIG. 13A, 31 cell types or sub-cell types were identified in Mount Sinai GSE83687 RNAseq Dataset with enrichment or reduction of certain cell types (or sub-types) in UC or CD patient tissues as compared control samples. Similarly, as shown in FIG. 13B, 34 cell types or sub-cell types were identified in Lloyd Price GSE111889 RNAseq Dataset with enrichment or reduction of certain cell types (or sub-types) in UC or CD patient tissues as compared control samples. Additionally, as shown in FIG. 13C, 20 cell types or sub-cell types were identified in UC119 with enrichment or reduction of certain cell types (or sub-types) in UC patient tissues. Additionally, as shown in FIG. 13D, 20 cell types or sub-cell types were identified in CD100 with enrichment or reduction of certain cell types (or sub-types) in CD patient tissues.

To further confirm the accuracy of the deconvolution, the cells deconvoluted from bulk RNA sequencing data from various IBD tissues were compared across the tissues. Because certain cell types are known to enrich in specific tissues in the gastrointestinal tract, the cell types deconvoluted from bulk RNA sequencing data is expected to show similar enrichment. As shown in FIG. 13E, the deconvoluted cell types and cell type proportions demonstrated that Paneth cells were enriched in small bowel and ileum over other tissues in the gastrointestinal tract, consistent with the known distribution of Paneth cells. Furthermore, as shown in FIG. 13F, the deconvoluted cell types and cell type proportions demonstrated that Paneth cells were enriched in intestinal tissues over rectum or colon, consistent with the known distribution of Paneth cells. Therefore, the deconvoluted cell types, sub-cell types, and their respective proportions were further validated by the expected cell type distribution across the tissues.

The deconvoluted cell types, sub-cell types and their respective proportions also demonstrated that certain cell types were enriched or reduced in diseased tissues of IBD patients over non-diseased control tissues. As shown in FIGS. 13G, 13H, 13I, and 13J, activated fibroblasts were enriched in both CD and UC diseased tissues as compared to non-diseased control tissues, based on deconvolution of Mount Sinai GSE83687 RNAseq Dataset (FIG. 13G), Lloyd Price GSE111889 RNAseq Dataset (FIG. 13H), UC119 (FIG. 13I), and CD100 (FIG. 13J). Similarly, monocyte derived dendritic cells (moDCs, FIGS. 13K-13N) and CD36+ endothelial cells (FIGS. 13O-13R) were enriched in both CD and UC diseased tissues as compared to non-diseased control tissues, based on deconvolution of Mount Sinai GSE83687 RNAseq Dataset (FIGS. 13K and 13O), Lloyd Price GSE111889 RNAseq Dataset (FIGS. 13L and 13P), UC119 (FIGS. 13M and 13Q), and CD100 (FIGS. 13N and 13R). On the other hands, BEST4+ enterocytes/epithelial cells (FIGS. 13S-13V) and Tuft cells (FIGS. 13W-13X) were reduced in CD and/or UC diseased tissues as compared to non-diseased control tissues, based on deconvolution of Mount Sinai GSE83687 RNAseq Dataset (FIGS. 13S and 13W), Lloyd Price GSE111889 RNAseq Dataset (FIGS. 13T and 13X), UC119 (FIG. 13U), and CD100 (FIG. 13V).

### 7.20.4 Subpart 4. Building and Validating SNP Models Based on the Association Between the SNP Models and the Up- or Down-Regulation of Certain Inflammatory Cell Types

As described above, a model for predicting therapeutic response to an inhibitor of TL1A activity or expression can use n number of the polymorphisms identified in the analyses provided in the Examples above and is referred to as n-SNP model, wherein n can be 1, 2, 3, 4, 5, 6, 7, 8, 9, 10, 11, 12, 13, 14, 15, 16, 17, 18, 19, 20, or more. (e.g. a model using 12 SNPs is a 12-SNP model, a model using 3 SNPs is a 3-SNP model, etc). A few methods can be adopted to identify the SNP models for predicting therapeutic response to a TL1A inhibitor treatment. Briefly, an exhaustive list of distinctive combinations of the candidate SNPs (e.g. SNPs from Table 27) can be formed and each distinctive combination of SNPs was used to train a CDx model using logistic regression to predict the binary outcome (remission or no remission after TL1A inhibitor treatment). The models from the exhaustive list of SNP combinations can be ranked by a performance metric, e.g. PPV, NPV, Sensitivity, Specificity, etc and the top models were determined based on the performance and selected for use in clinic along with the TL1A inhibitor therapy for IBD patients. For example, for each of the exhaustive list of all possible combinations of the 16 SNPs in Table 27, a logistic regression model was trained to classify the binary outcome (response or no response to TL1A inhibitor treatment) and the performance of the trained model was ranked by PPV value.

An alternative process similar to that described in the previous paragraph can also be adopted to arrive at the trained models with the various SNP combinations, in which the logistic regression is substituted with other supervised or unsupervised machine learning classifiers. The process described above uses a logistic regression with no penalty applied to the cost function to regularize the model. Any supervised machine learning classifier capable of taking SNPs as input features or covariates, and predicting a probability of a binary outcome, or alternatively the binary outcome (either true or false, 0 or 1, yes or no, etc.), is interchangeable in the aforementioned process with logistic regression. The disclosure provides that several well-established alternative classifier methods can be used to substituted logistic regression, including, but are not limited to, naïve bayes, perceptron, logistic regression with elastic net or lasso regularization, random forest, support vector machine (SVM), deep neural network, and XGBoost, all of which were known, available, and practiced in the field of data science and machine learning. These various processes, although different in computational efficiency and intensity, can often arrive at the same top performing SNP combination models.

In the event a different classifier than logistic regression is used, every preceding and successive step in the entire process of SNP selection and classifier training is identical, except for the process of obtaining beta coefficients from the trained model. This is because only some models are describable via beta coefficients, whereas other models are described using different mathematical representations. For example, a linear SVM is represented as a hyperplane that maximally separates the outcomes. The weights of the SVM model define the coordinates of a vector which is orthogonal to the hyperplane.

A supervised machine learning model may also be used to perform regression of a continuous score, rather than classification (e.g. response/no response; remission/no remission binary classification). This continuous score may be associated with the odds of the treatment response outcome, e.g., a higher score is associated with higher odds of response to anti-TL1A therapeutic agent provided herein. Such a continuous score could be a clinical evaluation or performance score or a continuous score from patients' objective markers, e.g. serum markers, fecal markers, scores from endoscopic image, etc. In the event a regression model is used instead of a logistic regression classifier, every preceding and successive step in the entire process of SNP selection and classifier training is identical, with the addition of another step to map the continuous outcome of the regression model against the binary outcome.

The disclosure further provides multiple ways of mathematically representing each SNP in the model, which can be optimized along with the optimization of the beta coefficient and/or the selection of the SNPs in the model. A more detailed description for the various ways of mathematically representing SNPs was provided in Tsepilov, Yakov A., et al. “Nonadditive effects of genes in human metabolomics.” Genetics 200.3 (2015): 707-718, which is incorporated herein in its entirety by reference.

TABLE 28
mathematical representation of a SNP in the model

| | Encoding Type | | Heterozygous | |
|---|---|---|---|---|
| | (Numeric | Homozygous | reference/ | Homozygous |
| | Encodings for | reference | alternative | alternative |
| | genotypes/ | alleles | alleles | alleles |
| | polymorphisms) | (Ref/Ref) | (Ref/Alt) | (Alt/Alt) |
| Representation | Additive | 0 | 1 | 2 |
| of SNPs | Ref Negative | 1 | 1 | 0 |
| in model | Alt Negative | 0 | 1 | 1 |
| | Alt Recessive | 0 | 0 | 1 |
| | Ref Recessive | 1 | 0 | 0 |
| | Haploinsuff | 0 | 1 | 0 |
| | (Haploinsufficient) | | | |

Because each of the encoding (the set of mathematical representations for Ref/Ref, Ref/Alt, and Alt/Alt as described in Table 28) may provide additional, non-redundant information, a PRI, MRS, or RPS can be calculated with a SNP simultaneously represented by one or more of encodings described in Table 28. When two different encodings were used in the model for one SNP and the two encodings provide identical information, the statistical analyses described above will lead to the β coefficient for one encoding being zero when both encodings were included in the statistical optimization. If two different encodings used in the model for one SNP provide non-redundant information, then the statistical analyses described above will lead to the β coefficients for both encodings being non-zero, thereby capturing signal from both encodings of that SNP in the model. Similar outcome applies when 3, 4, 5, or 6 encodings were used to simultaneously represent one SNP in a CDx model, in which the statistical analyses described herein determines that the signal from certain SNP encodings have already being captured, thus leading to the β being set to zero for these SNP encodings as shown in column 1 of Table 31. Therefore, any one or more (e.g. 1, 2, 3, 4, 5, 6, 7, 8, 9, 10 or more) SNPs or all SNPs in a model can be simultaneously represented in the model by 1, 2, 3, 4, 5, or 6 of encodings described in Table 28. In some examples, any one or more or all SNPs in the CDx model can be represented in the model by 1 encoding described in Table 28. In some examples, any one or more or all SNPs in the CDx model can be represented in the model by 2 encodings described in Table 28. In some examples, any one or more or all SNPs in the CDx model can be represented in the model by 3 encodings described in Table 28. In some examples, any one or more or all SNPs in the CDx model can be represented in the model by 4 encodings described in Table 28. In some examples, any one or more or all SNPs in the CDx model can be represented in the model by 5 encodings described in Table 28. In some examples, any one or more or all SNPs in the CDx model can be represented in the model by 6 encoding described in Table 28.

Applying the process described above, a large number of trained models with various combinations of SNPs shown in Table 27 along with various mathematical representation of the SNP described in Table 28 were generated and evaluated. For example, trained models from the exhaustive list of distinctive combinations of SNPs shown in Table 27 were generated, evaluated, and ranked. The resulting top performing models along with the parameters and performing metrics were described in Table 31.

For each model, a model risk score (“MRS”) can be calculated with the parameters described for each SNP, wherein in some examples the MRS=weighted summation of the SNPs in the model or

$$MRS = {β}_{0} + {\sum }_{i = 1}^{n} ( {β}_{i} ) \times ( {χ}_{i} ) ,$$

wherein χᵢ is the mathematical representation of the ith SNP in the model, which can be any one of the examples listed in Table 28. The performance of each model for predicting a patient response to an inhibitor of TL1A activity or expression can be assessed by plotting the specificity of the model against the sensitivity of the model (e.g. a receiver operating characteristic curve, a “ROC” curve) and evaluating the area under the curve (“AUC”) in such ROC curve. Alternatively, the performance of each model can be assessed by calculating the precision (positive predictive value, or precision, calculated as TP/(TP+FP) where is TP and FP are true positives and false positives) of SNP models in the patient cohort, plotting the precision against the sensitivity (also referred to as recall in the literature), and calculating the area under the precision-recall curve. Additionally, the performance of the model can be evaluated, ranked, and selected by measuring the actual PPV, NPV, specificity, accuracy, sensitivity, etc, with the clinical data, because the actual status of response or remission for each patient treated with TL1A inhibitor was known in the dataset generated as described in Section 7.22. Alternatively, the enrichment or reduction of certain IBD-related cell types in the IBD patient sample (as described in this Example) is used instead to assess how the model performed for predicting the enrichment or reduction of such IBD-related cell types, and for selecting the top performing models for clinical validation with actual IBD patients' response or remission. Similarly, the increase of TL1A expression in the immune cells in IBD patient sample (as described in Sections 7.4 to 7.6) is used instead to assess how the model performed for predicting the increase of TL1A expression in such IBD-related immune cell types, and for selecting the top performing models for clinical validation with actual IBD patients' response or remission.

Additionally, a logistic function can be applied to the MRS score to generate a continuous Response Probability Score (“RPS”), wherein in some RPS equals to 1/(1+e^{(-MRS)}) and ranges from 0 to 1.

Both RPS and MRS are examples of PRI, which can be used as described below to determine whether the subject is likely to respond to the treatment a treatment with an inhibitor of TL1A expression or activity. Briefly, to determine whether a patient is likely to responsive to a treatment with an inhibitor of TL1A expression or activity (e.g. TL1A CDx positive or TL1A CDx negative), various cutoff is adopted and for each the PRI (e.g. MRS or RPS) and for each cutoff adopted, a number of performing metrics of that PRI model with the adopted cutoff can be calculated, including positive rate (positivity), sensitivity, specificity, positive predictive value (PPV), negative predictive value (NPV), and balanced accuracy. These performing metrics can be plotted across the whole spectrum of the cutoff to determine the optimal cutoff based on the plot, for example, as further described below.

A few methods were adopted and compared to determine the cutoff value for the MRS that was used for selecting patients responsive to a treatment with an inhibitor of TL1A expression or activity (e.g. TL1A CDx positive or TL1A CDx negative). Two of these methods give equal weight to sensitivity and specificity with no ethical, cost and prevalence constraints. The first method uses the square of distance between the point (0, 1) on the upper left hand corner of ROC space and any point on ROC curve, i.e. d²=(1−sensitivity)²+(1−specificity)². In order to obtain the optimal cut off points, the square of this distance is minimized. In other words, this distance for each point on the ROC curve from the point (0, 1) can be calculated to find the optimal cut-off value. The second method called Youden index uses the maximum of vertical distance of ROC curve from the point (x, y) on diagonal line (chance line). Youden index maximizes the difference between sensitivity and (1−specificity): Youden Index=Sensitivity+Specificity−1. Thus, by maximizing Sensitivity+Specificity across various points on the ROC curve, the optimal cut-off point is calculated. The third method incorporates the financial costs for correct and false diagnosis and the costs of further work up for diagnosis. In fact, the consequence of each possible test outcome is ascertained to their costs and combining ROC analysis with utility-based decision theory can be used to determine the optimal cutoff. For example, given a disease with low prevalence and high cost of false positive diagnosis, the cutoff may be chosen at higher value to maximize specificity while for a disease occurring at high prevalence and missing diagnosis has a serious fatal consequences, a lower cutoff value would be selected to maximize sensitivity. In a specific example, the cutoff value is set for a sensitivity of 100%, 95%, 90%, 85%, 80%, 75%, 70%, 65%, 60%, 55%, 50%, 45%, 40%, 35%, 30%, 25%, or 20%. In another example, the cutoff value can be set for a pre-determined positive predictive value, e.g. 95%, 90%, 85%, 80%, 75%, 70%, 65%, 60%, 55%, 51%, 50%, 45%, 40%, 35%, 34%, 33%, 32%, 3%, 30%, 29%, 28%, 27%, 26%, r 25%. In other examples, the cutoff value can be set for a pre-determined positive rate, e.g. 30-40%. In one example, the cutoff value is set for a positive rate of about 25%. In one example, the cutoff value is set for a positive rate of about 26%. In one example, the cutoff value is set for a positive rate of about 27%. In one example, the cutoff value is set for a positive rate of about 28%. In one example, the cutoff value is set for a positive rate of about 29%. In one example, the cutoff value is set for a positive rate of about 30%. In one example, the cutoff value is set for a positive rate of about 31%. In one example, the cutoff value is set for a positive rate of about 32%. In one example, the cutoff value is set for a positive rate of about 33%. In one example, the cutoff value is set for a positive rate of about 34%. In one example, the cutoff value is set for a positive rate of about 35%. In one example, the cutoff value is set for a positive rate of about 36%. In one example, the cutoff value is set for a positive rate of about 37%. In one example, the cutoff value is set for a positive rate of about 38%. In one example, the cutoff value is set for a positive rate of about 39%. In one example, the cutoff value is set for a positive rate of about 40%. In one example, the cutoff value is set for a positive rate of about 41%. In one example, the cutoff value is set for a positive rate of about 42%. In one example, the cutoff value is set for a positive rate of about 43%. In one example, the cutoff value is set for a positive rate of about 44%. In one example, the cutoff value is set for a positive rate of about 45%. In the fourth method, the MRS score is calculated as described above and the logistic function is then applied to the MRS to generate a continuous Response Probability Score (“RPS”), wherein in some examples RPS equals to 1/(1+e^{(-MRS)}) and ranges from 0 to 1. In some examples, the model is trained, optimized, and normalized (e.g. normalization of β coefficient) such that patients in the training cohort that responded to TL1A inhibitor therapy would have a RPS value 0.5 and patients in the training cohort that did not respond to TL1A inhibitor therapy would have a RPS value≤0.5. Accordingly, 0.5 can be the cutoff for the RPS for determining whether a patient would be a responder or non-responder to TL1A inhibitor therapy. If the RPS is ≥0.5, the prediction is “yes, responder”. If the RPS is <0.5, the prediction is “no, non-responder”.

Having generated and evaluated CDx models using various combination of the candidate SNPs in the previous Examples (e.g. Table 27), the inventors identified a list of 8-SNP, 7-SNP, 6-SNP, 5-SNP, 4-SNP, 3-SNP, 2-SNP, or 1-SNP high performing models for predicting remission to treatment with an inhibitor of TL1A expression or activity. The parameters and performance of these models were summarized in Table 31.

In order to validate the model for predicting upregulation of IBD-related cell types and/or with remission in response to anti-TL1A treatment described in the Examples, the dataset of the patient cohorts were randomly split into training cohort (70% of the data) and testing/validating cohort (the remaining 30% of the data). The training cohort (70% of the data) was used to train logistic regression models, determine the coefficients, and determine a probabilistic threshold (above which the prediction is remission after treatment, and below which the prediction is no remission after treatment). In some examples, such training was performed with a logistic regression model with no regularization, and the limited-memory Broyden-Fletcher-Goldfarb-Shanno algorithm was used to solve the optimization problem. Such a trained model was applied to the held-out test/validating cohort (the remaining 30% of the data) to confirm the predictions, calculated the PPV, and calculate the various other performing metrics for the classifier (upregulation of IBD-related cell types as described in this Example or remission in response to anti-TL1A treatment generated from Section 7.22). Such a five-fold cross validation help minimize the bias in the set of metrics describing classification performance of the models, confirming that a model developed using the given combination of SNPs generalizes and performs well on predicting treatment response of data that the model has never seen. In such a testing/validating process, the dataset was randomly shuffled and divided into five bins with similar numbers of data per bin. Such cross validation helps minimize the bias in the set of metrics describing classification performance of the models, confirm that a model developed using the given combination of SNPs generalizes and performs well on predicting results (upregulation of IBD-related cell types as described in this Example or remission in response to anti-TL1A treatment generated from Section 7.22) that the model has never seen. This process was repeated 100 times. The mean and standard deviation of various classification performing metrics across the 100 test/validating sets were computed. Such metrics include positive rate (positivity), sensitivity, specificity, positive predictive value (PPV), negative predictive value (NPV), area under the receiver operating curve (AUROC), area under the precision recall curve (AUPRC), and balanced accuracy (geometric mean of recall, or sensitivity, across all labels or treatment response phenotypes), among others, as shown in Table 31.

Lengthy table referenced here
US20260066079A1-20260305-T00001
Please refer to the end of the specification for access instructions.

As shown in Table 31, the vast majority of the SNP models in Table 31 (65.4% or 1157 out of the 1770 models) have a PPV equal to or more than 70% and 700 of the SNP models in Table 31 have a PPV equal to or more than 60% (1356 out of 1770) for predicting IBD enriched cell types and/or MBD depleted cell types, thereby predicting a remission to TL1A inhibitor treatment.

Additionally, for all the models in Table 31 that were developed to predict an increase of a particular IBD-related cell type or cell sub-type, a merged model can be established by combining all the SNP encodings for that particular cell type and averaging all β coefficients for the same SNP encoding. Such merged models for each of ileum entero_clonocyte (enterocytes and clonocytes), ileum BEST4+ epithelial cells, ileum IgG plasma cells, ileum resident macrophages, ileum highly activated T cells, ileum microfold cells, ileum myofibroblasts, ileum activated fibroblasts, ileum CD36 endothelial cells, ileum lymphatic epithelial cells (lymphatics), colon TA cells, colon Panetch cells, colon Goblet cells, colon moDCs, colon IgG plasma cells, colon resident macrophages, colon EECs, ileum moDCs, and ileum enterocytes were listed in the last 19 rows. Such merged models can capture all the signals captured in each individual model and are tested in clinical trials described in Section 7.22.

### 7.21 Example 21: A SNP Genotyping Assay

This assay is used to identify the 8 SNPs in the 8 SNP models of Example 19. DNA from a sample derived from a subject is combined with primers specific to each SNP, PCR reagents, a wildtype probe and a mutant probe. Wildtype probes are tagged with Hex at the 5′ end and quencher dye at the 3′ end. Mutant probes are tagged with FAM at the 5′ end and a quencher at the 3′ end.

For each SNP to be tested, 4 standard reactions are run: no template, homozygous wildtype, heterozygous, and homozygous mutant. Standard reactions use templates comprising a gblock comprising all 8 WT or mutant SNP sequences. For the heterozygous standard reaction the template comprises both the WT gblock and the mutant gblock.

Samples are run in duplicate. The reactions are run and analyzed using QuantStudioDx software to detect the genotypes of each sample. Genotypes are identified as homozygous wildtype, heterozygous, or homozygous mutant for each SNP and sample tested.

### 7.22 Example 22: A Phase 2, Multi-Center, Double-Blind, Placebo-Controlled Study to Evaluate the CDx for Treatment with an Anti-TL1A Antibody in Subjects with Moderately to Severely Active Ulcerative Colitis

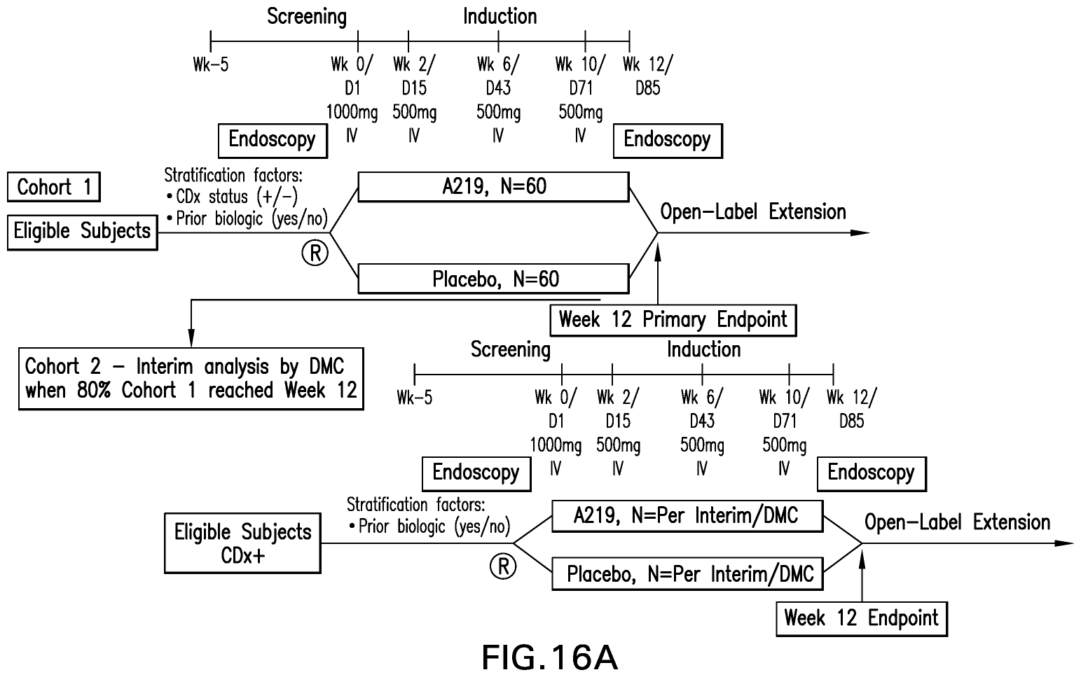

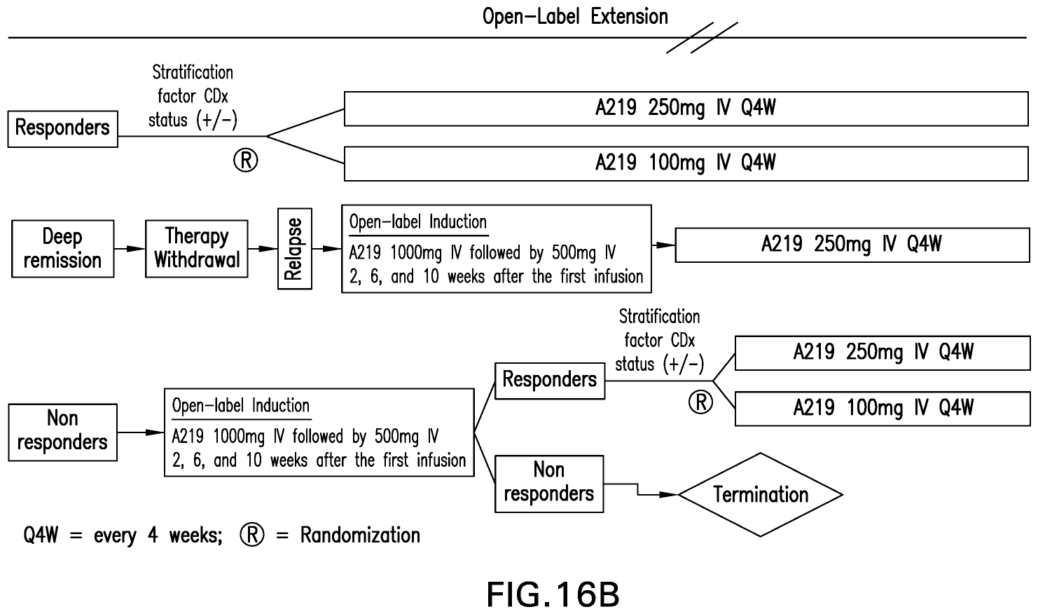

To validate the efficacy of the CDx treatment with anti-TL1A antibodies in ulcerative colitis (UC), a phase 2 clinical trial is conducted. The detailed design of the clinical trial protocol is provided in the protocol synopsis of Table 26 below and shown in FIGS. 16A-16B.

TABLE 26
Protocol Synopsis

| | |
|---|---|
| TITLE: | A Phase 2, Multi-Center, Double-Blind, Placebo-Controlled Study to |
| | Evaluatethe CDx for Treatment with an Anti-TL1A Antibody in |
| | Subjects with Moderately to Severely Active Ulcerative Colitis |
| PROJECT | Phase 2 |
| PHASE: | |
| OBJECTIVE: | Primary: |

To assess the safety and tolerability of A219 following 12-
weeks ofinduction therapy
To compare the efficacy of A219 vs placebo for induction of
clinicalremission at Week 12

Secondary:
All objectives below refer to comparison of A219-treated subjects
vs placebo-treated subjects in Cohort 1. For the objectives where
the companion diagnostic (CDx) status is a variable, a
comparison of subjectsin both Cohort 1 and Cohort 2 will be
conducted.

To compare the efficacy of A219 vs placebo for
induction ofendoscopic improvement at Week 12
To compare the efficacy of A219 vs placebo for induction of
clinicalresponse at Week 12
To compare the efficacy of A219 vs placebo in CDx
positive (CDx+) subjects (Cohort 1 + Cohort 2) for
induction of clinical remission at Week 12
To compare the efficacy of A219 vs placebo for induction
ofhistologic remission at Week 12
To compare the efficacy of A219 vs placebo for induction
ofhistologic-endoscopic mucosal improvement at Week
12
To compare the efficacy of A219 vs placebo in CDx+
subjects (Cohort 1 + Cohort 2) for induction of endoscopic
improvement atWeek 12
To compare the efficacy of A219 vs placebo in CDx+
subjects (Cohort 1 + Cohort 2) for induction of clinical
response at Week 12
To compare the efficacy of A219 vs placebo in CDx+ subjects
(Cohort 1 + Cohort 2) for induction of histologic remission at
Week 12
To compare the efficacy of A219 vs placebo in CDx+
subjects (Cohort 1 + Cohort 2) for induction of histologic-
endoscopic mucosalimprovement at Week 12
To compare the efficacy of A219 in CDx+ (Cohort 1 + Cohort
2) vsCDx negative (CDx−) subjects for induction of clinical
remission at Week 12
To compare the efficacy of A219 vs placebo for
induction ofmucosal healing at Week 12
To compare the efficacy of A219 vs placebo in CDx+
subjects (Cohort 1 + Cohort 2) for induction of mucosal
healing at Week 12
To compare the efficacy of A219 vs placebo for change
in Inflammatory Bowel Disease Questionnaire (IBDQ)
at Week 12
To compare the efficacy of A219 vs placebo in CDx+
subjects(Cohort 1 + Cohort 2) for change in IBDQ at
Week 12
To compare the efficacy of A219 vs. placebo in subjects
who areCDx+ per alternative algorithm (Cohort 1 + Cohort
2) for clinical remission at Week 12

Exploratory:

To assess the pharmacokinetics (PK) of A219 in subjects
withulcerative colitis (UC) over time
To assess the effects of A219 on tissue and serum
pharmacodynamic(PD) markers, including total TL1A
concentrations over time
To assess the effect of A219 on inflammatory biomarkers
including fecal calprotectin and high sensitivity C-reactive
protein (hsCRP) overtime
To assess the proportion of subjects in 3-component
Modified Mayo Score response, 3-component Modified
Mayo Score remission, endoscopic improvement, Robarts
histopathology index (RHI) histologic remission, Geboes
score histologic remission, and mucosalhealing at Week 50
To assess the change in Partial Mayo Score over time
To assess the change in Geboes Index and RHI from
Baseline toWeek 12 and Week 50
To assess the exposure-response relationship of A219 on PD
markersover time
To assess the proportion of subjects achieving
corticosteroid-free-remission at Week 50

| | |
|---|---|
| STUDY | This is a multi-center, double-blind, randomized, placebo- |
| DESIGN: | controlled proof of concept study designed to assess the safety, |
| | tolerability, and efficacy of A219 following 12 weeks of induction |
| | therapy in subjects with UC. Thisstudy will be conducted under the |
| | aegis of a Data Monitoring Committee (DMC) and will commence |
| | following the demonstration of an acceptable safety profile of A219 |
| | at a dose of ≥500 mg in the multiple ascending dose study in |
| | normal healthy volunteers (Example 16). |
| | The study has 4 periods (Screening Period, Induction Period [IP], |
| | Open- Label Extension [OLE] Period, and Follow-Up [FU] |
| | Period). The study will have 2 Cohorts that will enroll subjects in a |
| | sequential fashion utilizingan adaptive design as described below. |
| | Cohort 1: Following the Screening Period, approximately 120 |
| | eligible subjects with moderately to severely active UC will enter |
| | the IP and be randomized in a 1:1 fashion to receive intravenous |
| | (IV) administration of A219 1000 mg on Week 0/Day 1, followed |
| | by 500 mg on Weeks 2, 6, and 10, or placebo at the same |
| | timepoints. Randomization will be stratifiedby CDx status of |
| | positive (CDx+) or negative (CDx−) and prior biologic experience |
| | (yes/no) at Week 0/Day 1. Subjects who discontinue from the |
| | study will have a follow-up period of 12 weeks after last dose. |
| | Cohort 2: When approximately 80% of Cohort 1 subjects (i.e., ~96 |
| | subjects)have reached Week 12 or early terminated from the study, |
| | the DMC will conduct an unblinded analysis of clinical efficacy in |
| | CDx+ subjects and willrecommend whether an expansion to |
| | Cohort 2 is warranted. The planned sample size for CDx+ subjects |
| | (combining Cohort 1 and Cohort 2) will be approximately 40, in |
| | the case where Cohort 2 is initiated. For Cohort 2, eligible subjects |
| | (who must be CDx+) will enter the IP and be randomized in a 1:1 |
| | fashion to receive IV administration of A219 1000 mg on |
| | Week 0/Day 1, followed by 500 mg on Weeks 2, 6, and 10, or |
| | placebo atthe same timepoints. Randomization will be stratified |
| | by prior biologic experience (yes/no) at Baseline. Subjects who |
| | discontinue from the studywill have a follow-up period of 12 |
| | weeks after last dose. |
| | Subjects who complete the 12-week IP from either Cohort will |
| | have theoption to enter OLE. During OLE, starting at Week 14 |
| | visit: |

Subjects who have achieved deep remission (defined as in
clinical remission [endoscopic subscore of 0 or 1, rectal
bleeding subscore of 0, and stool frequency subscore of 0 or 1
and not greater than Baseline] and RHI < 3) will continue in the
study and have therapy withdrawal.

```
Upon disease relapse (defined as rectal bleeding score
  of ≥1, and either hsCRP ≥5 and/or fecal calprotectin ≥250),
  subjects can receive another course of induction therapy
  (1000 mg IV followedby 500 mg IV 2, 6, and 10 weeks
  after the first infusion) followed by maintenance therapy
  of 250 mg IV every 4 weeks (Q4W) for atotal of 50
  weeks.
```

Responders (defined as reduction from Baseline ≥2 points
and ≥30% in 3-component Modified Mayo Score, accompanied by
a reduction ≥1in rectal bleeding subscore or absolute rectal
bleeding subscore ≤1) who have not achieved deep remission
will be re-randomized, stratifiedby CDx status of CDx+ or
CDx−, to either 250 mg IV Q4W or 100 mg IV Q4W, starting at
Week 14 until Week 50.
Nonresponders will receive an open-label induction regimen of
1000 mg of A219 on Week 14, followed by 500 mg on
Weeks 16, 20, and 24. Nonresponders who do not respond at
Week 28 (per investigator discretion) should be discontinued
from the study.
Nonresponders who respond at Week 28 (per investigator
discretion) will be re-randomized to either 250 mg IV Q4W
or 100 mg IV Q4W, starting at Week 28 for a total of 50
weeks.

| | |
|---|---|
| | The study may be amended by the Sponsor to extend the |
| | OLE periodbeyond 50 weeks based on emerging safety data. |
| | The study also includes an optional PK sub-study during the IP for |
| | subjectswho consent to additional PK sampling. |
| SAMPLE | The study is planned to randomize up to approximately 170 |
| SIZE: | subjects, approximately 120 in Cohort 1 and up to 50 in Cohort 2. |
| | A sample size of60 per arm in Cohort 1 will enable a statistical |
| | power of >80% for the primary endpoint at 1-sided significance |
| | level of 0.025 using Cochran- Mantel-Haenszel (CMH), |
| | assuming clinical remission rate of 5% for placebo and 24% for |
| | A219. Additionally, the sample size will confer >80% |
| | power to achieve statistical significance for the 1^{st} |
| | secondary endpoint of endoscopic improvement with an overall 1- |
| | sided alpha level of0.025, assuming the endoscopic improvement |
| | rates are 15% and 38% for placebo and A219 groups, respectively. |
| | Additionally, for analyses of the CDx population (combining |
| | CDx+ subjects from Cohort 1 and Cohort 2), a sample size of 40 |
| | per arm will provide a statistical power of ≥80% at a 1-sided |
| | alpha level of 0.025, according to a group sequential design with a |
| | non-binding futility interimanalysis when 18 subjects per arm |
| | reach Week 12, assuming clinical remission rate of 5% for placebo |
| | and 31% for A219. |
| SUBJECT | Male or female subjects ≥18 years of age with moderately to |
| TYPE: | severelyactive UC. |
| FORMULATIONS: | A219 will be supplied in 10 mL vials each containing 500 mg |
| | A219(60 mg/mL solution) for IV administration after |
| | reconstitution. |
| DOSAGE: | Subjects will be stratified by CDx+/CDx− status and prior biologic |
| | experience (yes/no) in a 1:1 ratio to: |

A219 1000 mg IV on Week 0/Day 1, followed by 500 mg
IV on Weeks 2, 6, and 10
Placebo IV on Week 0/Day 1, followed by placebo IV on
Weeks 2, 6, and 10

During OLE:

Deep remitters will enter the therapy withdrawal period

```
All subjects who develop disease relapse after therapy
  withdrawal will receive another course of induction therapy
  (1000 mg IV followed by 500 mg IV 2, 6, and 10 weeks
  after the first infusion) followed by maintenance therapy of
  250 mg IV Q4W for a total of 50weeks.
```

Responders at the end of Week 12 will be stratified by CDx
status ofCDx+ or CDx− and re-randomized to receive one of
the following regimens:

```
  A219 250 mg IV on Week 14 then Q4W until Week 50
  A219 100 mg IV on Week 14 then Q4W until Week 50
```

Nonresponders at the end of Week 12 will receive open-label
A219 1000 mg IV on Week 14, followed by A219 500 mg IV
on Weeks 16, 20, and 24. Subjects who do not respond by
Week 26 should be discontinued from the study. Subjects who
respond by Week 26 will bestratified by CDx status of CDx+ or
CDx− and re-randomized to receiveone of the following
regimens:

```
  A219 250 mg IV on Week 28 then Q4W for a total of 50
  weeks
  A219 100 mg IV on Week 28 then Q4W for a total of 50
  weeks
```

| ROUTE OF | All study drug will be reconstituted in 250 mL of 0.9% normal | |
|---|---|---|
| ADMINISTRATION: | saline (NS)and will be administered IV over 30 minutes. | |
| STUDY | Primary endpoints: | |
| ENDPOINTS: | | The proportion of subjects reporting adverse events (AEs), |
| | | serious adverse events (SAEs), AEs leading to |
| | | discontinuation, and markedly abnormal laboratory values. |
| | | The proportion of subjects in the 3-component Modified Mayo |
| | | Score clinical remission (as defined by endoscopic subscore of |
| | | 0 or 1, rectal bleeding subscore of 0, and stool frequency |
| | | subscore of 0 or 1 and not greater than Baseline) at Week 12. |
| | | The 3-component Modified Mayo Score ranges from 0-9 and |
| | | includes rectal bleeding, stool frequency andendoscopic |
| | | assessment domains. |
| | Secondary endpoints: | |
| | | The proportion of subjects with endoscopic improvement, as |
| | | defined byendoscopy subscore ≤1 with no friability) at Week |
| | | 12. |
| | | The proportion of subjects in 3-component Modified Mayo Score |
| | | clinicalresponse at Week 12. The 3-component Modified Mayo |
| | | Score clinical response is defined by reduction from Baseline ≥2 |
| | | points and ≥30% in3-component Modified Mayo Score, |
| | | accompanied by a reduction ≥1 inrectal bleeding subscore or |
| | | absolute rectal bleeding subscore ≤1. |
| | | The proportion of subjects in the 3-component Modified Mayo |
| | | Score clinical remission (as defined by endoscopic subscore of |
| | | 0 or 1, rectal bleeding subscore of 0, and stool frequency |
| | | subscore of 0 or 1 and not greater than Baseline), in CDx+ |
| | | subjects (Cohort 1 + Cohort 2) treatedwith A219 compared to |
| | | CDx+ placebo-treated subjects at Week 12. The 3-component |
| | | Modified Mayo Score ranges from 0-9 and includes rectal |
| | | bleeding, stool frequency, and endoscopic assessment domains. |
| | | The proportion of subjects with histologic remission (defined |
| | | Geboesscore ≤3.1) at Week 12. |
| | | The proportion of subjects with histologic-endoscopic |
| | | mucosal improvement (defined as Geboes score ≤3.1 and |
| | | endoscopy subscore ≤1 |
| | | with no friability) at Week 12. |
| | | The proportion of subjects with endoscopic improvement, as |
| | | defined byendoscopy subscore ≤1 with no friability, in CDx+ |
| | | subjects (Cohort 1 + |
| | | Cohort 2) treated with A219 compared to CDx+ placebo- |
| | | treatedsubjects at Week 12. |
| | | The proportion of subjects in 3-component Modified Mayo |
| | | Score clinical response in CDx+ subjects treated with A219 |
| | | compared to CDx+ placebo-treated subjects at Week 12. The 3- |
| | | component ModifiedMayo Score clinical response is defined |
| | | by reduction from Baseline ≥2points and ≥30% in 3- |
| | | component Modified Mayo Score, accompanied by a reduction ≥1 |
| | | in rectal bleeding subscore or absolute rectal bleeding |
| | | subscore ≤1. |
| | | The proportion of subjects with histologic remission, defined as |
| | | Geboesscore ≤3.1, in CDx+ subjects (Cohort 1 + Cohort 2) |
| | | treated with A219 compared to CDx+ placebo-treated subjects |
| | | at Week 12. |
| | | The proportion of subjects with histologic-endoscopic |
| | | mucosal improvement (defined as Geboes score ≤3.1 and |
| | | endoscopy subscore ≤1 |
| | | with no friability), in CDx+ subjects (Cohort 1 + Cohort 2) |
| | | treatedwith A219 compared to CDx+ placebo-treated subjects |
| | | at Week 12. |
| | | The proportion of subjects with clinical remission (defined as |
| | | endoscopic subscore of 0 or 1, rectal bleeding subscore of 0, |
| | | and stoolfrequency subscore of 0 or 1 and not greater than |
| | | Baseline) in CDx+ subjects (Cohort 1 + Cohort 2) treated |
| | | with A219 compared to in CDx− subjects treated with A219 at |
| | | Week 12. |
| | | The proportion of subjects with mucosal healing (defined as |
| | | Geboesscore ≤2B.1 and endoscopy subscore of ≤1) at |
| | | Week 12. |
| | | The proportion of subjects with mucosal healing (defined as |
| | | Geboesscore ≤2B.1 and endoscopy subscore of ≤1), in |
| | | CDx+ subjects (Cohort 1 + Cohort 2) treated with A219 |
| | | compared to CDx+ placebo-treated subjects at Week 12. |
| | | The proportion of subjects with IBDQ response, as defined |
| | | by ≥16-point increase from Baseline at Week 12. |
| | | The proportion of subjects with IBDQ response, as defined |
| | | by ≥16- point increase from Baseline, in CDx+ subjects |
| | | (Cohort 1 + Cohort 2)treated with A219 compared to CDx+ |
| | | placebo-treated subjects at Week 12. |
| | | The proportion of subjects in the 3-component Modified |
| | | Mayo Score clinical remission (as defined by endoscopic |
| | | subscore of 0 or 1, rectalbleeding subscore of 0, and stool |
| | | frequency subscore of 0 or 1 and notgreater than Baseline), in |
| | | CDx+ subjects per alternative algorithm (Cohort 1 + Cohort |
| | | 2) treated with A219 compared to CDx+ |
| | | placebo-treated subjects per alternative algorithm at Week 12. |
| | | The 3-component Modified Mayo Score ranges from 0-9 and |
| | | includes rectal bleeding, stool frequency, and endoscopic |
| | | assessment domains. |
| | Exploratory endpoints: | |
| | | The pharmacokinetics of A219 in subjects with UC after |
| | | multipledoses |
| | | The change from Baseline in serum and fecal inflammatory |
| | | biomarkers(PD markers) |
| | | The proportion of subjects in 3-component Modified Mayo |
| | | Score response, 3-component Modified Mayo Score |
| | | remission, endoscopicimprovement, RHI histologic |
| | | remission, Geboes score histologic remission, and mucosal |
| | | healing at Week 50 |
| | | The change in Partial Mayo Score (with or without PGA |
| | | component)over time |
| | | The change in Geboes Index and RHI from Baseline to |
| | | Week 12 andWeek 50 |
| | | The exposure-response relationship of A219 on PD markers |
| | | Within subpopulation of subjects on corticosteroid at study |
| | | entry, the proportion of subjects in clinical remission and off |
| | | of corticosteroid atWeek 50 |
| INCLUSION | Subjects are required to meet the following criteria in order to be | |
| CRITERIA: | included inthe study: | |
| | 1. | Male or female ≥18 years of age. |
| | 2. | Subjects must have had a documented diagnosis of UC |
| | | (endoscopy +histology) to be eligible for study participation. |
| | | For subjects with no documented confirmation of UC |
| | | diagnosis or if previous diagnosis isnot deemed conclusive, |
| | | UC diagnosis must be confirmed at time of screening |
| | | colonoscopy. |
| | 3. | Moderately to severely active UC as defined by 3-component |
| | | ModifiedMayo score (3 components of rectal bleeding, stool |
| | | frequency, and endoscopy) of 4 to 9, inclusive, with Modified |
| | | Mayo endoscopic subscore ≥2 and rectal bleeding subscore ≥1. |
| | 4. | Subjects must satisfy at least one of the following criteria: |

| a) | In the past, had an inadequate response to one or more | |
|---|---|---|
| | of thefollowing treatments: | |
| | | Oral prednisone ≥40 mg/day (or equivalent) or |
| | | budesonide ≥9 mg/day or equivalent or beclomethasone ≥5 |
| | | mg/day for at least2 weeks |
| | | Corticosteroid dependence as defined by failed to |
| | | successfully taper to <10 mg/day of prednisone or |
| | | equivalent (i.e., had a |
| | | flare of disease) within 3 months of starting |
| | | therapy, or if relapse occurs within 3 months of |
| | | stopping corticosteroids |
| | | Immunosuppressants (azathioprine ≥2 mg/kg/day or |
| | | 6-mercaptopurine ≥1.0 mg/kg/day [or documentation |
| | | of a therapeutic concentration of 6-thioguanine |
| | | nucleotide]) for atleast 8 weeks |
| | | An approved anti-TNF agent at an approved labeled |
| | | dose for atleast 8 weeks |
| | | Vedolizumab at the approved labelled dose for at least 8 |
| | | weeks |
| | | An approved JAK inhibitor (e.g., tofacitinib) at an |
| | | approvedlabelled dose for at least 8 weeks |
| | | An approved anti-IL-12/23 (e.g., ustekinumab) at an |
| | | approvedlabelled dose for at least 8 weeks |
| | | An approved sphingosine 1-phosphate receptor |
| | | (S1PR) modulator at an approved labelled dose for |
| | | least 12 weeks |
| OR | | |

| b) | Had been intolerant to one or more of the above- |
|---|---|
| | mentioned treatments (e.g., unable to achieve doses or |
| | treatment durations because of dose limiting side effects |
| | [e.g., leukopenia, psychosis, uncontrolled diabetes, |
| | elevated liver enzymes]). |
| OR | |
| c) | Currently receiving one or more of the following treatments: |

Oral Prednisone ⊐ 10 mg/day (or equivalent) for at least 3
months
Immunosuppressants [azathioprine ≥2 mg/kg/day or
6-mercaptopurine □ 1.0 mg/kg/day (or documentation
of a therapeutic concentration of 6-thioguanine
nucleotide)] for atleast 8 weeks.

Notes on subjects who have had prior biologic/biologic-like
therapy(ies) (anti-TNF, JAK inhibitor, SIPR modulator, anti-
IL-12/23, and/or vedolizumab):

```
  The study will include a maximum of 70% subjects who
  have had prior biologic/biologic-like therapy(ies)
  experience. Upon reachingthe maximum number of
  allowed biologic/biologic-like experienced subjects (70%),
  subjects who have had prior biologic/biologic-like
  experience will no longer be allowed to enterthe study.
  Subject cannot have failed (no response, insufficient
  response, lossof response, and/or intolerance) >3 classes
  or >4 individual biologic/biologic-like therapies (refer to
  exclusion criterion #26).
```

| | |
|---|---|
| 5. | For subjects who are women of childbearing potential |
| | (WOCBP) involved in any sexual intercourse that could lead |
| | to pregnancy, the subject has used two highly effective |
| | methods of contraception for atleast 4 weeks prior to Day 1 |
| | and agrees to continue to use two highly |
| | effective methods of contraception until at least 12 weeks after |
| | the lastdose of study drug. |
| 6. | Male subjects must use, with their female partner of |
| | childbearing potential, two highly effective methods of |
| | contraception and refrain from sperm donation from screening |
| | to 12 weeks after the last dose ofstudy drug. |
| 7. | Subject must meet drug stabilization requirements, as |
| | applicable: |

| | |
|---|---|
| a) | Oral corticosteroid treatment must be equivalent of ≤20 |
| | mg prednisone or ≤9 mg budesonide or beclomethasone ≤5 |
| | mg dailyat a stable dose for at least 2 weeks prior to |
| | randomization |
| b) | Oral aminosalicylates should be at a stable dose for at least |
| | 2 weeksprior to randomization |
| c) | Azathioprine and 6-mercaptopurine should be at a stable |
| | dose for atleast 4 weeks prior to randomization |

| | 8. | Able to provide written informed consent and understand and |
|---|---|---|
| | | comply with the requirements of the study. |
| | 9. | For Cohort 2 only: Subjects must be CDx+. |
| EXCLUSION | Subjects with the following characteristics will be excluded from | |
| CRITERIA: | the study: | |
| | Sex and Reproductive Status | |
| | 1. | WOCBP and men with female partners of childbearing |
| | | potential who are unwilling or unable to use two highly |
| | | effective methods of contraception to avoid pregnancy for the |
| | | entire study period and for upto 12 weeks after the last dose of |
| | | study drug. |
| | 2. | Women who are pregnant or breastfeeding. |
| | 3. | Women with a positive pregnancy test on enrollment or |
| | | prior torandomization. |
| | Target Disease Exceptions | |
| | 4. | Diagnosis of Crohn's disease or indeterminate colitis. |
| | 5. | UC limited to the rectum (<15 cm from anal verge). |
| | 6. | Current evidence of fulminant colitis, toxic megacolon, |
| | | or bowelperforation. |
| | 7. | Current or impending need for colostomy or ileostomy. |
| | 8. | Previous total proctocolectomy or partial colectomy. |
| | 9. | Surgical bowel resection within 3 months before screening. |
| | 10. | Concomitant primary sclerosing cholangitis (PSC) |
| | Medical History and Concurrent Diseases | |
| | 11. | Past or current evidence of definite low-grade or high-grade |
| | | colonicdysplasia that has not been completely removed. |
| | 12. | Subjects who are scheduled or anticipate the need for |
| | | surgery, asidefrom dermatologic procedures. |
| | 13. | Subjects who have a history of clinically significant drug or |
| | | alcoholabuse. |
| | 14. | Concomitant illness that in the opinion of the Investigator, is |
| | | likely torequire systemic glucocorticosteroid therapy during |
| | | the study (e.g., moderate to severe asthma). |
| | 15. | Current symptoms of severe, progressive, or uncontrolled |
| | | renal, hepatic, hematological, pulmonary, cardiac, |
| | | neurological, ophthalmologic or cerebral disease. |
| | | Concomitant medical conditionsthat in the opinion of the |
| | | Investigator might place the subject at unacceptable risk for |
| | | participation in this study. |
| | 16. | Subjects with a history of cancer within the last 5 years (other |
| | | than non-melanoma skin cell cancers cured by local |
| | | resection). Existing non-melanoma skin cell cancers must be |
| | | removed prior to enrollment. Subjects with carcinoma in situ |
| | | or localized cervical cancer, treated with definitive surgical |
| | | intervention, are allowed. |
| | 17. | Subjects at risk for tuberculosis (TB). Specifically, subjects |
| | | with: |

| | |
|---|---|
| a) | A history of active TB |
| b) | Current clinical, radiographic or laboratory evidence of |
| | active TB |
| c) | Latent TB which was not successfully treated. Subjects |
| | with a positive TB screening test indicative of latent TB |
| | will not be eligible for the study unless active TB infection |
| | has been ruled out, and an appropriate course of |
| | intervention for latent TB has been initiated at least 2 |
| | weeks prior to randomization, and no evidence of active |
| | TB on chest x-ray during Screening. |

| 18. | Subjects with any serious bacterial infection within the last 3 |
|---|---|
| | months, unless treated and resolved with antibiotics, or any |
| | chronic bacterial infection (such as chronic pyelonephritis, |
| | osteomyelitis and bronchiectasis). |
| 19. | Female subjects who have had a breast cancer screening that is |
| | suspicious for malignancy, and in whom the possibility of |
| | malignancy cannot be reasonably excluded following |
| | additional clinical, laboratoryor other diagnostic evaluations. |
| 20. | Subjects with any active infections (excluding fungal |
| | infections of nailbeds) including, but not limited to, those that |
| | require intravenous (IV) antimicrobial treatment 4 weeks or |
| | oral antimicrobial treatment 2 weeks prior to randomization. |
| | Subjects with evidence of Human Immunodeficiency Virus |
| | (HIV), Hepatitis B or Hepatitis C infection detected during |
| | screening are also excluded, but subjects with successfully |
| | treated Hepatitis C with no recurrence for ≥1 year are |
| | allowed. Subjects with active documented or suspected |
| | COVID-19 |
| | infection within 4 weeks of randomization or asymptomatic |
| | SARS-CoV-2 PCR test within 2 weeks of randomization are |
| | excluded. |
| 21. | Subjects with herpes zoster reactivation or cytomegalovirus |
| | (CMV) that resolved less than 2 months prior to signing |
| | informed consent. |
| 22. | Subjects who have received any live vaccines within 3 months |
| | of theanticipated first dose of study medication or who will |
| | have need of alive vaccine at any time during the study. |
| Physical and Laboratory Test Findings | |
| 23. | Positive stool Polymerase Chain Reaction (PCR) or culture for |
| | entericpathogens. |
| 24. | Stool positive for Clostridium difficile (C. difficile) toxin. |
| | Subjects whoare positive can be retested after the completion |
| | of a full course of treatment for C. difficile infection. |
| 25. | Any of the following lab values: |

| | |
|---|---|
| a) | Hemoglobin (Hgb) < 8.0 g/dL (80 g/L) |
| b) | White blood cell (WBC) < 2,500/mm³ (2.5 × 10⁹/L) |
| c) | Neutrophils < 1,000/mm³ (1 × 10⁹/L) |
| d) | Platelets < 100,000/mm³ (100 × 10⁹/L) |
| e) | Serum creatinine >2 times upper limit of normal (ULN) |
| f) | Serum alanine aminotransferase (ALT) or |
| | aspartateaminotransferase (AST) >2 times |
| | ULN |
| g) | Any other laboratory test results that, in the opinion |
| | of the Investigator, might place the subject at |
| | unacceptable risk forparticipation in this study |

| Prohibited Therapies and/or Medications | |
|---|---|
| 26. | Failed (no response, insufficient response, loss of response, |
| | and/or intolerance) >3 classes (anti-TNF, anti-integrin, |
| | anti-IL12/23, JAKinhibitor, S1PR modulator) or >4 |
| | individual biologic/biologic-liketherapies. |
| 27. | Any marketed biologic or biologic-like within 2 weeks for |
| | tofacitinib, 8 weeks for anti-TNF agents, 10 weeks for SIPR |
| | modulators, and 12 weeks for vedolizumab and ustekinumab |
| | prior to randomization or if drug level per therapeutic dose |
| | monitoring is greater than lower limit ofdetection. |
| 28. | Any biologic immunomodulators not covered in exclusion |
| | criterion 27, used for UC or other conditions within 8 weeks or |
| | 5 half-lives, whichever is longer, prior to randomization or if |
| | drug level per therapeutic dose monitoring is greater than lower |
| | limit of detection. |
| 29. | Rituximab within 1 year prior to randomization. |
| 30. | Parenteral corticosteroids within 4 weeks or rectal |
| | administration ofcorticosteroids within 2 weeks prior to |
| | randomization. |
| 31. | Rectal administration of 5-ASA within 2 weeks prior to |
| | randomization. |
| 32. | Tacrolimus, methotrexate, cyclosporine, mycophenolate mofetil |
| | (CellCept ®), immunoadsorption columns (such as Prosorba |
| | columns), D Penicillamine, Leflunomide, Thalidomide, fish-oil |
| | preparations, probiotics, fecal transplantation, non-steroidal |
| | anti-inflammatory agents(NSAIDs), aspirin >81 mg/day within |
| | 2 weeks prior to randomization. |
| 33. | Other investigational chemical agent within 30 days or other |
| | investigational biologic agent within 8 weeks or 5 half-lives |
| | (whicheveris longer) of randomization. |
| 34. | Prior exposure to A219. |
| Other Exclusion Criteria | |
| 35. | Prisoners or subjects who are compulsorily detained |
| | (involuntarily incarcerated) for treatment of either a |
| | psychiatric or physical (e.g.,infectious disease) illness must |
| | not be enrolled into this study. |
| | Legal or mental incapacitation, or inability to understand and |
| | complywith the requirements of the study |

| | |
|---|---|
| Statistical | Statistical methods will be detailed in the Statistical Analysis Plan. |
| Methods: | The SAP will provide details about the method of analysis and |
| | specific plannedanalyses, and will be prepared and approved by |
| | Prometheus Biosciences and its designees before study database |
| | lock and unblinding of subject treatment assignments. |
| | The analysis populations are defined as follows: |

Full analysis set (FAS) from Cohort 1: all subjects
randomized andtreated in Cohort 1
FAS for CDx+: all CDx+ subjects who are randomized and
treated inboth Cohort 1 and Cohort 2
Safety analysis set: all subjects

treated The following analyses will
be performed:
Efficacy:
The efficacy assessment will test for the difference between
A219 andplacebo groups in FAS.
The primary endpoint will be analyzed and compared between
A219 andplacebo treatment groups in FAS from Cohort 1. The
primary endpoint, the proportion of subjects achieving clinical
remission, will be tested between the 2 treatment groups at 1-sided
significance level of 0.025 using CMH with stratification factors at
randomization. If significant, the 1^{st} secondary endpoint of
proportion of subjects achieving endoscopic improvement will be
tested between the 2 treatment groups at 1-sided significance level
of 0.025. If significant, the 2^{nd}, 3^{rd}, 4^{th}, and 5^{th} secondary endpoints
will be tested sequentially, each at 1-sided significance level of
0.025. Testing forstatistical significance will stop when the first
endpoint is not statistically significant at level of 0.025 and all
remaining p values will be nominal.
Treatment difference for primary and secondary endpoints for
Cohort 1 willbe estimated along with 95% CI for all subjects in
FAS. The secondary endpoints in CDx+ subjects will be
summarized and compared between A219 and placebo groups in
FAS for CDx+, while the treatment difference will be estimated
with 95% CI. Additional efficacy analysis will be detailed in SAP.
Interim Efficacy Analysis:
An interim analysis will be carried out when approximately 80%
of subjects (approximately 96 subjects) in Cohort 1 have reached
Week 12 orearly terminated from the study. The DMC will review
the unblinded efficacy and safety data and recommend on the
expansion to Cohort 2.
Decision rules to initiate Cohort 2 are determined according to the
futility bounds of group sequential design of a sample size of 40
per arm with oneinterim analysis at the information fraction of
45%. Because the exact bounds will be calculated using the actual
number of subjects with CDx+ included in the interim analysis,
the final decision rules, along with sensitivity analysis, will be
specified in the DMC SAP, prior to the interimanalysis.
Adverse Events:
AEs will be coded using the most current version of Medical
Dictionary forRegulatory Activities (MedDRA^{⊐}).
A by-subject AE data listing, including verbatim term, preferred
term (PT), system organ class (SOC), treatment, severity,
seriousness criteria, relationship to drug, and action taken, will be
provided.
The number of subjects experiencing treatment-emergent
adverse events(TEAEs) and number of TEAEs will be
summarized by treatment using frequency counts for Safety
analysis set.
Medical History, chest x-ray, electrocardiogram (ECG), and
physicalexamination will be listed by subject.
Changes in ECGs and physical examinations will be described in
the text ofthe final report.
Concomitant Medications:
Concomitant medications will be coded using the most
current WorldHealth Organization (WHO) drug dictionary
and listed by treatment.
Pharmacokinetics:
Summary statistics of A219 concentrations and anti-drug
antibody(ADA) by visit and by CDx+ and CDx−.

### 7.23 Example 23: Validation of CDx in a Phase 2a, Multi-Center, Open-Label Clinical Trial of A219 in Subjects with Moderately to Severely Active Crohn's Disease

To validate the effectiveness of the CDx models described in Section 7.20 in selecting Crohn's disease (CD) patients responsive to treatment of an anti-TL1A antibody, a phase 2 clinical trial with a representative anti-TL1A antibody, A219 as described herein, and the CDx models described in Section 7.20 is conducted. The detailed design of the clinical trial protocol is shown in the protocol synopsis in Table 32 below.

TABLE 32
Protocol Synopsis of Phase 2A Trial for CDx and A219 in CD Patients

| | |
|---|---|
| TITLE: | A Phase 2a, Multi-Center, Open-Label Study to Evaluate the Safety, |
| | Efficacy, and Pharmacokinetics of A219 in Subjects with Moderately to |
| | Severely Active Crohn's Disease |
| PROJECT PHASE: | Phase 2a |
| OBJECTIVE | Primary: |
| | To evaluate the safety and tolerability of A219 following 12-weeksof |
| | induction therapy |
| | To assess the proportion of subjects with endoscopic improvement |
| | (decrease in simple endoscopy score for Crohn's disease [SES-CD] ≥50% |
| | from Baseline) at Week 12 |
| | Secondary: |
| | To assess the proportion of subjects with clinical remission (Crohn's |
| | disease activity index [CDAI] <150) at Week 12 |
| | To assess the proportion of subjects with endoscopy and clinical |
| | improvement (decrease in SES-CD ≥50% AND reduction in |
| | CDAI ≥100 points from Baseline) at Week 12 |
| | To assess the proportion of subjects with clinical remission at Week |
| | 12 and/or with endoscopic improvement (each as defined in the above two |
| | clauses) among the subjects that tested positive in the CDx described in |
| | Section 7.20 |
| | To assess the proportion of subjects with biomarker and clinical |
| | improvement (decrease in high sensitivity C-reactive protein [hsCRP] or |
| | fecal calprotectin ≥50% from Baseline, among subjects with at leastone |
| | elevated biomarker at Baseline, AND reduction in CDAI ≥100 points from |
| | Baseline) at Week 12 |
| | ≥100 points from Baseline) at Week 12 |
| | To assess the proportion of subjects with normalization of C-reactive |
| | protein (hsCRP < upper limit of normal [ULN]), among subjects with |
| | elevated concentrations at Baseline, at Week 12 |
| | To assess the proportion of subjects with normalization of fecal |
| | calprotectin (fecal calprotectin < ULN), among subjects with elevated |
| | concentrations at Baseline, at Week 12 |
| | To assess the proportion of subjects with clinical improvement |
| | (reduction in CDAI ≥100 points from Baseline) at Week 12 |
| | To assess the proportion of subjects with two component patient- |
| | reported outcome (PRO-2) remission (average daily abdominal pain score ≤1 |
| | point and average daily stool frequency ≤3 points with abdominal pain and |
| | stool frequency no worse than Baseline at Week 12) |
| | To assess the change in SES-CD score from Baseline to Week 12 |
| | To assess the pharmacokinetics (PK) of A219 |
| | To assess the immunogenicity of A219 |
| | Exploratory: |
| | To assess the change in CDAI and component scores over time |
| | To assess the effects of A219 on tissue and serum pharmacodynamic |
| | (PD) markers, including tumor necrosis factor-like cytokine 1A (TL1A) |
| | concentrations, endoscopic healing index (EHI), fecal calprotectin, and |
| | hsCRP in all subjects over time |
| | To assess the change in SES-CD at Week 50 from Baseline |
| | To characterize the change in Perianal Disease Activity Index (PDAI) |
| | score from Baseline to Week 12, Week 28, and Week 50 |
| | To characterize the effect of A219 for improvement and remissionof |
| | enterocutaneous and/or perianal fistula during the Induction Period(IP) and |
| | Open-Label Extension (OLE) |
| | To assess all secondary endpoints at Week 50 |
| | To assess the change in global histological activity score (GHAS) and |
| | Robarts histopathology index (RHI) from Baseline to Week 12 and Week |
| | 50 |
| | To assess the proportion of subjects with histologic response and |
| | histologic remission at Week 12 and Week 50 |
| | To assess change in PRO-2 over time |
| | To assess change in extraintestinal manifestations over time |
| STUDY DESIGN: | This is a multi-center, open-label, proof of concept study designed to assess |
| | the safety, tolerability, and preliminary efficacy of A219 following |
| | 12 weeks of induction therapy in subjects with Crohn's disease (CD). This |
| | study will be conducted under the aegis of a Data Monitoring Committee |
| | (DMC) and will commence following the demonstration of an acceptable |
| | safety profile of A219 at a dose of ≥500 mg in the multiple ascending dose |
| | study in normal healthy volunteers (Study PR200-101). |
| | The study has 4 periods (Screening, IP, OLE and Follow-Up [FU] Period). |
| | Following the Screening Period, approximately 50 eligible subjects with |
| | moderately to severely active CD will enter the IP to receive A219 1000 |
| | mg on Week 0/Day 1, followed by 500 mg on Weeks 2, 6, and 10 via |
| | intravenous (IV) administration. Subjects who discontinue from the study |
| | will have a follow-up period of 12 weeks after the last dose. |
| | Response at Week 12 will be defined as reduction from Baseline in CDAI |
| | of ≥100 points. Non-responders at Week 12 should discontinue from the |
| | study. |
| | Subjects who complete the 12-week IP and have responded will have the |
| | option to enter OLE. Subjects will continue in OLE until they progress, |
| | withdraw from the study, study termination, or Week 50. During the OLE, |
| | starting at Week 14 visit: |
| | Subjects who achieved deep remission (defined as clinical remission |
| | with CDAI <150, endoscopic remission with SES-CD ≤4 with none of the |
| | segments with score of more than 1, and RHI <3) will continuein the study |
| | and have therapy withdrawal |
| | Upon disease relapse (defined as CDAI ≥220 and either hsCRP ≥5 |
| | mg/L and/or fecal calprotectin ≥250 μg/g), subjects can receive another |
| | course of induction therapy (1000 mg IV followedby 500 mg IV 2, 6, and |
| | 10 weeks after the first infusion) followed by maintenance therapy of 250 |
| | mg IV every 4 weeks (Q4W) for atotal of 50 weeks |
| | Other subjects will be re-randomized to either 250 mg IV Q4W or |
| | 100 mg IV Q4W until Week 50 |
| | The study may be amended by the Sponsor to extend the OLE period |
| | beyond 50 weeks based on emerging safety data. |
| SAMPLE SIZE: | The study is planned to enroll approximately 50 subjects. The sample size |
| | will enable a statistical power of 80%, at 1-sided significance level of 0.025, |
| | to test against the null hypothesis of endoscopic improvement rate of12%, |
| | assuming the endoscopic improvement rate for A219 is 27%. |
| SUBJECT TYPE: | Male or female subjects ≥18 years of age with moderately to severely |
| | active CD. |
| FORMULATIONS: | A219 will be supplied in 10 mL vials each containing 500 mg A219(60 |
| | mg/mL solution) for IV administration after reconstitution. |
| DOSAGE: | Induction Period: all subjects will receive A219 1000 mg on Week |
| | 0/Day 1, followed by 500 mg IV on Weeks 2, 6, and 10. |
| | Non-responders at Week 12 should be discontinued from the study. |
| | Responders at the end of Week 12 have the option to enter the OLE, where |
| | subjects with deep remission will undergo therapy withdrawal and subjects |
| | without deep remission will be randomized to receive one of the following |
| | regimens until disease progression, withdraw from the study, study |
| | termination, or Week 50: |
| | A219 250 mg IV on Week 14 then Q4W |
| | A219 100 mg IV on Week 14 then Q4W |
| | Subjects who do not respond by the end of Week 12 should be discontinued |
| | from the study. |
| | All subjects who develop disease relapse (defined as CDAI ≥220 and either |
| | hsCRP ≥5 mg/L and/or fecal calprotectin >250 ug/g), after therapy |
| | withdrawal will receive another course of induction therapy (1000 mg IV |
| | followed by 500 mg IV 2, 6, and 10 weeks after the first infusion) followed |
| | by maintenance therapy of 250 mg IV Q4W for a total of 50 weeks. |
| ROUTE OF | The study drug will be reconstituted in 250 mL of 0.9% normal saline (NS) |
| ADMINISTRATION: | and will be administered IV over 30 minutes. |
| STUDY ENDPOINTS: | Primary endpoints: |
| | Safety and tolerability: the proportion of subjects reporting adverse |
| | events (AEs), serious adverse events (SAEs), AEs leading to |
| | discontinuation, and markedly abnormal laboratory values |
| | The proportion of subjects with endoscopic improvement, as defined by |
| | decrease in SES-CD ≥50% from Baseline at Week 12 |
| | Secondary endpoints: |
| | The proportion of subjects in clinical remission (CDAI <150) at |
| | Week 12 |
| | The proportion of subjects with endoscopic and clinical improvement, |
| | as defined by decrease in SES-CD ≥50% AND reduction in CDAI ≥100 |
| | points from Baseline at Week 12 |
| | The proportion of subjects with both biomarker and clinical |
| | improvement (decrease in hsCRP OR fecal calprotectin ≥50% from |
| | Baseline, among subjects with at least one elevated biomarker at |
| | Baseline, AND reduction in CDAI ≥100 points from Baseline) at Week |
| | 12 |
| | The proportion of subjects with normalization of hsCRP (as defined by |
| | hsCRP < ULN), among subjects with elevated concentrations at Baseline, at |
| | Week 12 |
| | The proportion of subjects with normalization of fecal calprotectin |
| | (as defined by fecal calprotectin < ULN), among subjects with elevated |
| | concentrations at Baseline, at Week 12 |
| | The proportion of subjects in clinical response, as defined by reduction |
| | in CDAI ≥100 points from Baseline at Week 12 |
| | The proportion of subjects with PRO-2 remission (defined as average |
| | daily abdominal pain score ≤1 point and average daily stool frequency ≤3 |
| | points with abdominal pain and stool frequency no worse than |
| | Baseline) at Week 12 |
| | Change in SES-CD score at Week 12 from Baseline |
| | Descriptive summaries of PK and immunogenicity of A219 |
| | Proportion of subjects developing anti-drug antibody (ADA) and |
| | neutralizing antibody (Nab) |
| | Exploratory endpoints: |
| | Change in CDAI and components from Baseline over time |
| | Change in PD markers including TL1A concentrations, EHI, fecal |
| | calprotectin, and hsCRP over time |
| | Change in SES-CD from Baseline at Week 50 |
| | Change in PDAI from Baseline over time |
| | Proportion of subjects with improvement or remission of |
| | enterocutaneous and/or perianal fistula at Week 12 and Week 50 |
| | Change in GHAS and RHI from Baseline to Week 12 and Week 50 |
| | The proportion of subjects with GHAS histologic score ≤4 at Week 12 |
| | and Week 50 |
| | The proportion of subjects with Robarts histologic score <5 at Week |
| | 12 and Week 50 |
| | The proportion of subjects with GHAS histologic remission, defined as |
| | no neutrophils in the epithelium or subscore of 0, at Week 12 and Week 50 |
| | The proportion of subjects with Robarts histologic remission (<3) at |
| | Week 12 and Week 50 |
| | Time to relapse among subjects with deep remission at Week 12 |
| | Proportion of subjects who relapse, among subjects who achieved deep |
| | remission at Week 12 |
| | Change in PRO-2 over time |
| | The proportion of subjects with extraintestinal manifestation through |
| | Week 50 |
| INCLUSION | Subjects are required to meet the following criteria in order to be included in |
| CRITERIA: | the study: |
| | 1. Male or female ≥18 years of age. |
| | 2. Subjects must have had a documented diagnosis of CD (endoscopy + |
| | histology) to be eligible for study participation. For subjects with no |
| | documented confirmation of CD diagnosis or if previous diagnosis isnot |
| | deemed conclusive, CD diagnosis must be confirmed at time of screening |
| | colonoscopy. |
| | 3. Moderately to severely active CD as defined by CDAI of ≥220 and ≤450. |
| | 4. SES-CD score (percentral reading) ≥6 if ileocolonic or colonic |
| | disease; or ≥4 if isolated ileal disease only. |
| | 5. Subjects must satisfy at least one of the following criteria: |
| | a) In the past, had an inadequate response to one or more of the |
| | following treatments: |
| | Oral prednisone ≥40 mg/day (or equivalent) or budesonide ≥9 |
| | mg/day or equivalent or beclomethasone ≥5 mg/day for atleast 2 weeks |
| | Corticosteroid dependence as defined by failed to successfully |
| | taper to <10 mg/day of prednisone or equivalent (i.e., had a flare of |
| | disease) within 3 months of starting therapy, or if relapse occurs within 3 |
| | months of stopping corticosteroids |
| | Immunosuppressants (azathioprine ≥2 mg/kg/day or |
| | 6-mercaptopurine ≥1.0 mg/kg/day, [or documentation of atherapeutic |
| | concentration of 6-thioguanine nucleotide] or methotrexate ≥15 |
| | mg/week) for at least 8 weeks |
| | An approved anti-TNF agent at an approved labeled dose for at |
| | least 8 weeks |
| | An approved anti-integrin (e.g., vedolizumab) at an approved |
| | labeled dose for at least 8 weeks |
| | An approved anti-IL-12/23 (e.g., ustekinumab) at an approved |
| | labeled dose for at least 8 weeks |
| | OR |
| | b) Had been intolerant to one or more_of the above mentioned |
| | treatments (e.g., unable to achieve doses or treatment durations because of |
| | dose-limiting side effects [e.g., leukopenia, psychosis, uncontrolled |
| | diabetes, elevated liver enzymes]) |
| | OR |
| | c) Currently receiving one or more of the following treatments: |
| | Oral Prednisone ≥20 mg/day (or equivalent) or budesonide ≥3 |
| | mg/day for at least 4 weeks |
| | Immunosuppressants [azathioprine ≥2 mg/kg/day or |
| | 6-mercaptopurine ≥1.0 mg/kg/day, (or documentation of a therapeutic |
| | concentration of 6-thioguanine nucleotide)] for atleast 8 weeks |
| | Notes on subjects who have had prior approved biologic therapy(ies)(e.g., |
| | anti-TNF, anti-integrin, and/or anti-IL-12/23): |
| | The study will include a maximum of 70% subjects who have had |
| | prior approved biologic therapy(ies) experience. Upon reaching the |
| | maximum number of allowed biologic experienced subjects (70%), subjects |
| | who have had prior biologic experience will no longer be allowed to enter |
| | the study. |
| | Subjects cannot have had failed (no response, insufficient response, |
| | loss of response, and/or intolerance) >4 approved biologic therapies, |
| | whether of same or different mechanism of action |
| | Subjects previously on clinical trials only (i.e., did not receive |
| | commercial available therapy post-approval) are not considered tohave |
| | received the approved therapy for purpose of this inclusion criteria |
| | 6. For subjects who are women of childbearing potential (WOCBP) involved |
| | in any sexual intercourse that could lead to pregnancy, the |
| | subject has used two highly effective methods of contraception for at least 4 |
| | weeks prior to Day 1 and agrees to continue to use two highly effective |
| | methods of contraception until at least 12 weeks after the lastdose of study |
| | drug. |
| | 7. Male subjects must use, with their female partner of childbearing |
| | potential, two highly effective methods of contraception and refrain from |
| | sperm donation from screening to 12 weeks after the last dose ofstudy drug. |
| | 8. Subjects must meet drug stabilization requirements, as applicable: |
| | a) Oral corticosteroid treatment must be equivalent of ≤20 mg |
| | prednisone or ≤9 mg budesonide or beclomethasone ≤5 mg dailyat a stable |
| | dose for at least 2 weeks prior to Day 1 |
| | b) Oral aminosalicylates should be at a stable dose for at least 2 |
| | weeks prior to Day 1 |
| | c) Azathioprine, 6-mercaptopurine, and methotrexate should be at a |
| | stable dose for at least 4 weeks prior to Day 1 |
| | 9. Able to provide written informed consent and understand and comply |
| | with the requirements of the study. |
| EXCLUSION | Subjects with the following characteristics will be excluded from the study: |
| CRITERIA: | Sex and Reproductive Status |
| | 1. WOCBP and men with female partners of childbearing potential who |
| | are unwilling or unable to use two highly effective methods of contraception |
| | to avoid pregnancy for the entire study period and for upto 12 weeks after |
| | the last dose of study drug. |
| | 2 . Women who are pregnant or breastfeeding. |
| | 3. Women with a positive pregnancy test on enrollment or prior to Day 1. |
| | Target Disease Exceptions |
| | 4. Diagnosis of ulcerative colitis or indeterminate colitis. |
| | 5 . CD isolated to the stomach, duodenum, jejunum, or perianal region, |
| | without colonic and/or ileal involvement. |
| | 6. Suspected or diagnosed intra-abdominal or perianal abscess at |
| | Screening. |
| | 7. Known symptomatic stricture or stenosis not passable in endoscopy |
| | (including pediatric colonoscope). |
| | 8. Current stoma or need for colostomy or ileostomy. |
| | 9. Previous small bowel resection with combined resected length of >100 |
| | cm or previous colonic resection of >2 segments. |
| | 10. Currently receiving total parenteral nutrition. |
| | 11. Surgical bowel resection within 3 months before screening. |
| | 12. Concomitant primary sclerosing cholangitis (PSC). |
| | Medical History and Concurrent Diseases |
| | 13. Past or current evidence of definite low-grade or high-grade colonic |
| | dysplasia that has not been completely removed. |
| | 14. Subjects who are scheduled or anticipate the need for surgery, aside |
| | from dermatologic procedures. |
| | 15. Subjects who have a history of clinically significant drug or alcohol |
| | abuse. |
| | 16. Concomitant illness that in the opinion of the Investigator, is likely |
| | to require systemic glucocorticosteroid therapy during the study (e.g., |
| | moderate to severe asthma). |
| | 17. Current symptoms of severe, progressive, or uncontrolled renal, |
| | hepatic, hematological, pulmonary, cardiac, neurological, ophthalmologic, |
| | or cerebral disease. Concomitant medical conditionsthat in the opinion of |
| | the Investigator might place the subject at unacceptable risk for |
| | participation in this study. |
| | 18. Subjects with a history of cancer within the last 5 years (other than |
| | non-melanoma skin cell cancers cured by local resection). Existing non- |
| | melanoma skin cell cancers must be removed prior to enrollment. Subjects |
| | with carcinoma in situ or localized cervical cancer, treated with definitive |
| | surgical intervention, are allowed. |
| | 19. Subjects at risk for tuberculosis (TB). Specifically, subjects with: |
| | a) A history of active TB |
| | b) Current clinical, radiographic, or laboratory evidence of active TB |
| | c) Latent TB which was not successfully treated. Subjects with a |
| | positive TB screening test indicative of latent TB will not be eligible for the |
| | study unless active TB infection has been ruled out, and an appropriate |
| | course of intervention for latent TB has been initiated at least 2 weeks prior |
| | to Day 1, and no evidence of active TB on chest x-ray during screening. |
| | 20. Subjects with any serious bacterial infection within the last 3 months, |
| | unless treated and resolved with antibiotics, or any chronic bacterial |
| | infection (such as chronic pyelonephritis, osteomyelitis, and |
| | bronchiectasis). |
| | 21. Female subjects who have had a breast cancer screening that is |
| | suspicious for malignancy, and in whom the possibility of malignancy cannot |
| | be reasonably excluded following additional clinical, laboratory,or other |
| | diagnostic evaluations. |
| | 22. Subjects with any active infections (excluding fungal infections of nail |
| | beds) including, but not limited to, those that require IV antimicrobial |
| | treatment 4 weeks or oral antimicrobial treatment 2 weeks prior to |
| | randomization. Subjects with evidence of Human Immunodeficiency |
| | Virus (HIV), Hepatitis B, or Hepatitis C infection detected during screening |
| | are also excluded, but subjects with successfully treated Hepatitis C with no |
| | recurrence for ≥1 year are allowed. Subjects with active documented or |
| | suspected COVID-19 infection within 4 weeks ofrandomization or |
| | asymptomatic SARS-CoV-2 PCR test within 2 weeksof randomization are |
| | excluded. |
| | 23. Subjects with herpes zoster reactivation or cytomegalovirus (CMV) |
| | that resolved less than 2 months prior to signing informed consent. |
| | 24. Subjects who have received any live vaccines within 3 months of the |
| | anticipated first dose of study medication or who will have need of alive |
| | vaccine at any time during the study. |
| | Physical and Laboratory Test Findings |
| | 25. Positive stool Polymerase Chain Reaction (PCR) or culture for enteric |
| | pathogens. |
| | 26. Stool positive for Clostridium difficile (C. difficile) toxin. Subjects who |
| | are positive can be retested after the completion of a full course of treatment |
| | for C. difficile infection. |
| | 27. Any of the following lab values: |
| | a) Hemoglobin (Hgb) <8.0 g/dL (80 g/L) |
| | b) White blood cell (WBC) <2,500/mm³ (2.5 × 10⁹/L) |
| | c) Neutrophils <1,000/mm³ (1 × 10⁹/L) |
| | d) Platelets <100,000/mm³ (100 × 10⁹/L) |
| | d) Serum creatinine >2 times ULN |
| | e) Serum alanine aminotransferase (ALT) or aspartate |
| | aminotransferase (AST) >2 times ULN |
| | f) Any other laboratory test results that, in the opinion of the |
| | Investigator, might place the subject at unacceptable risk for |
| | participation in this study. |
| | Prohibited Therapies and/or Medications |
| | 28. Failed (no response, insufficient response, loss of response, and/or |
| | intolerance) >4 approved biologic therapies (anti-TNF, anti-integrin, anti- |
| | IL12/23), whether of same or different mechanism of action. |
| | 29. Any marketed biologic within 8 weeks for anti-TNF agents and 12 |
| | weeks for anti-integrin agents (e.g., vedolizumab) and ustekinumabprior |
| | to Day 1 or if drug level per therapeutic dose monitoring is greater than |
| | lower limit of detection. |
| | 30. Any biologic immunomodulators used for CD or other conditions within |
| | 8 weeks or 5 half-lives, whichever is longer, prior to Day 1 or ifdrug level |
| | per therapeutic dose monitoring is greater than lower limit ofdetection. |
| | 31. Rituximab within 1 year prior to Day 1. |
| | 32. Parenteral corticosteroids within 4 weeks or rectal administration of |
| | corticosteroids within 2 weeks prior to Day 1. |
| | 33. Rectal administration of 5-ASA within 2 weeks prior to Day 1. |
| | 34. Tacrolimus, cyclosporine, mycophenolate mofetil (CellCept ®), |
| | immunoadsorption columns (such as Prosorba columns), D Penicillamine, |
| | Leflunomide, Thalidomide, chronic use of non-steroidalanti-inflammatory |
| | agents (NSAIDs), and aspirin >81 mg/day within 2 weeks prior to Day 1. |
| | 35. Other investigational chemical agent within 30 days or other |
| | investigational biologic agent within 8 weeks or 5 half-lives (whicheveris |
| | longer) of entry into the IP. |
| | 36. Prior exposure to A219. |
| | Other Exclusion Criteria |
| | 37. Prisoners or subjects who are compulsorily detained (involuntarily |
| | incarcerated) for treatment of either a psychiatric or physical |
| | (e.g., infectious disease) illness. |
| | 38. Legal or mental incapacitation, or inability to understand and comply |
| | with the requirements of the study. |
| Statistical | Statistical methods will be detailed in the Statistical Analysis Plan (SAP). |
| Methods: | The SAP will provide details about methods of analysis and the specific |
| | planned analyses, and will be prepared and approved by Prometheus |
| | Biosciences and its designees before study database lock. |
| | The analysis populations are defined as follows: |
| | Full analysis set (FAS): all subjects treated with Baseline SES-CD score |
| | Safety analysis set: all subjects treated |
| | The following analyses will be performed: |
| | Efficacy: |
| | The primary efficacy endpoint, endoscopic improvement at Week 12, will be |
| | used to assess the efficacy of A219. The proportion of subjects in FAS with |
| | endoscopic improvement will be tested against the null hypothesis of |
| | endoscopic improvement rate of 12%, at a 1-sided significance level of |
| | 0.025. If significant, the 1^{st} secondary endpoint of proportion of subjects |
| | achieving clinical remission will be tested against thenull hypothesis of |
| | clinical remission rate of 16%, at a 1-sided significance level of 0.025. |
| | The point estimates for the primary and secondary endpoints will be |
| | calculated along with 90% confidence interval for FAS and by companion |
| | diagnostic (CDx) status (CDx+ or CDx−). |
| | Adverse Events: |
| | AEs will be coded using the most current version of Medical Dictionary for |
| | Regulatory Activities (MedDRA ®). |
| | A by-subject AE data listing, including verbatim term, preferred term (PT), |
| | system organ class (SOC), treatment, severity, seriousness criteria, |
| | relationship to drug, and action taken, will be provided. |
| | The number of subjects experiencing treatment-emergent adverse events |
| | (TEAEs) and number of TEAEs will be summarized by treatment using |
| | frequency counts in safety analysis set. |
| | Medical History, chest x-ray, electrocardiogram (ECG), and physical |
| | examination will be listed by subject. |
| | Changes in ECGs and physical examinations will be described in the text of |
| | the final report. |
| | Concomitant Medications: |
| | Concomitant medications will be coded using the most current World |
| | Health Organization (WHO) drug dictionary and listed by treatment. |
| | Pharmacokinetics: |
| | Summary statistics of A219 concentrations and ADA by visit. |

As described above, the disclosure established various CDx models predicting altered regulation of colon moDCs, colon Paneth cells, colon resident macrophages, colon TA cells, colon goblet cells, colon IgG plasma cells, colon EECs, colon entero-clonocytes, ileum BEST4±epithelial cells, ileum entero_clonocytes, ileum highly activated T cells, ileum IgG plasma cells, ileum lymphatics, ileum myofibroblasts, ileum activated fibroblasts, ileum CD36+ endothelial cells, ileum moDCs, ileum microfold cells, and/or ileum resident macrophages. The disclosure provides that the TL1A-driven pathology in IBD patients (e.g. CD and UC patients) can be mediated by one of these cell types and thus the CDx models predicting IBD patients with one or more of such cell types can predict the clinical response of the TBD patients after treatment by anti-TL1A antibodies.

A number of CDx models from Table 31 were tested and successfully validated in clinical trial described in this Example. Briefly, a total of 53 CD patients were recruited and completed the clinical trial with anti-TL1A (A219) treatment described in Table 32. 26 of the 53 (49%) CD patients had clinical remission after anti-TL1A (A219) treatment as defined in the Primary Objective in Table 32. 13 of the 53 (25%) CD patients had endoscopic improvement after anti-TL1A (A219) treatment as defined in the Primary Objective in Table 32. All CD patients were genotyped (e.g. for genotypes of SNPs in Table 27) and a number of CDx models from Table 31 were applied to determine the CDx status (CDx positive or CDx negative) based on the genotypes of the CD patients. Based on the CDx status, the proportion of the CD patients with clinical remission and/or with endoscopic improvement (each as defined in Table 32) among CDx positive and CDx negative patients were calculated and compared with the proportion among all patients. The performing metrics of various CDx models (including positive rate (positivity), sensitivity, specificity, positive predictive value (PPV), negative predictive value (NPV), area under the receiver operating curve (AUROC) and balanced accuracy) in the clinical trial were shown in Table 33 (based on clinical remission in CD patients as defined in Table 32) and Table 34 (based on endoscopic improvement as defined in Table 32).

As shown in Table 34, the vast majority of CDx models predictive of colon moDC, ileum moDC, ileum entero_clonocyte, ileum enterocytes, colon resident macrophages, colon goblet cells, and colon TA cells from Table 31 enriched patients having endoscopic improvement when comparing with the proportion in all patients (24.5% in all patients, and 537/622 or 86% of the models resulted in PPV>25.5%). The patients having endoscopic improvement was enriched to as high as over 60% in some CDx models, comparing to the 24.5% with endoscopic improvement in all patients. Similarly, as shown in Table 33, a large number of CDx models predictive of colon moDC, ileum moDC, ileum entero_clonocyte, ileum enterocytes, colon resident macrophages, colon goblet cells, and colon TA cells from Table 31 enriched patients having clinical remission when comparing with the proportion in all patients, resulting in clinical remission as high as over 60% in CDx+ population in some CDx models.

TABLE 33
Clinical validation (based on clinical remission) of CDx models predictive of colon moDC, ileum moDC, ileum entero_clonocyte, ileum enterocytes, colon resident macrophages, colon goblet cells, and
colon TA cells from Table 31. In Table 33, PPV, NPV, sensitivity, specificity, accuracy, precision, and AUC are all calculated based on actual clinical remission in CD patients treated with anti-TL1A
(A219 antibody) as clinical remission is defined in Table 32. In Table 33, the β coefficients in column 1 are listed in a sequential order to match the sequential order of the corresponding SNP
and SNP encodings in the SNP combination shown in the second column. In column 2 of Table 33, the numeric encoding of the SNPs (additive, Ref Negative, Alt Negative, Alt Recessive, Ref Recessive, and
Haploinsufficient (Haploinsuff)) are as described in Tabe 28. Additionally, in column 2 of Table 33, all SNPs are listed with chromosome number and hg19 location, therefore, as examples,
9:117568766 = rs6478109; 14:98734567 = rs1892231; 21:45658474 = rs56124762; 5:11562221 = rs16901748; 16:11331509 = rs12934476; 20:62305274 = rs2297437; 11:128443099 = rs7935393;
16:6147645 = rs9806914. Each individual SNP and its SNP encoding in column 2 is separate by a comma from another SNP and SNP encoding. As described in in the disclosure, and SNP can be represented
by more than one numeric encodings in the model, as shown in column 2 of Table 33. Metrics not determined are left blank in the table. The last column of Table 33 lists the diseased tissue and the
cell types based on which the CDx models were developed from as described in Table 31 and the accompanying text. For determining CDx positive or negative, RPS was calculated with the β coefficients
of column 1 and the SNP encodings of column 2 and a cutoff of 0.5 was used (CDx+ when RPS ≥ 0.5; and CDx− when RPS < 0.5). The Ref and Alt alleles of the SNPs and the SNP encoding
described in column 2 are provided in Table 1. “num snp” in column 3 describes the number of SNPs in the model, wherein each SNP is counted only once (e.g. for a 3-SNP model in which the first
SNP is represented by 2 numeric encoding as indicated in column 2, the second and third SNPs are represented by 1 numeric encoding as indicated in column 2, the num snp will be 3).

| | SNPs and SNP encodings | | | | | | | | | | |
|---|---|---|---|---|---|---|---|---|---|---|---|
| | (one SNP can have 1, 2, | | | | | | | | | | |
| | 3, 4, 5, or 6 encoding in | | | | | | | | | speci- | |
| Beta (β) | the model) | num.snp | positivity | accuracy | auc | precision | npv | ppv | sensitivity | ficity | model |
| 0.00959 | ref.negative.5:11562221, | 5 | 0.37254902 | 0.54901961 | 0.56076923 | 0.57894737 | 0.53125 | 0.57894737 | 0.42307692 | 0.68 | colon.bma.model.cluster.Goblet.cell.RDS |
| 0.01394 | ref.negative.20:62305274, | | | | | | | | | | |
| −0.00194 | alt.negative.9:117568766, | | | | | | | | | | |
| 0.00921 | additive.9:117568766, | | | | | | | | | | |
| 0.0041 | ref.negative.14:98734567, | | | | | | | | | | |
| 0.00255 | ref.negative.21:45658474 | | | | | | | | | | |
| 0.00700823864361131 | ref.negative.5:11562221, | 5 | 0.37254902 | 0.54901961 | 0.55923077 | 0.57894737 | 0.53125 | 0.57894737 | 0.42307692 | 0.68 | colon.bma.model.cluster.Goblet.cell.RDS |
| 0.00592471300741337 | ref.negative.20:62305274, | | | | | | | | | | |
| 0.00250681408805735 | additive.9:117568766, | | | | | | | | | | |
| 0.00394667129254054 | alt.negative.9:117568766, | | | | | | | | | | |
| 0.00131460936552099 | ref.negative.14:98734567, | | | | | | | | | | |
| 0.00132408089547359 | ref.negative.21:45658474 | | | | | | | | | | |
| 0.01252 | ref.negative.5:11562221, | 4 | 0.70588235 | 0.64705882 | 0.65153846 | 0.61111111 | 0.73333333 | 0.61111111 | 0.84615385 | 0.44 | colon.bma.model.cluster.Goblet.cell.RDS |
| 0.01075 | ref.negative.20:62305274, | | | | | | | | | | |
| 0.00588 | ref.negative.14:98734567, | | | | | | | | | | |
| 0.00697 | ref.negative.21:45658474 | | | | | | | | | | |
| 0.00837 | ref.negative.5:11562221, | 4 | 0.39215686 | 0.56862745 | 0.55153846 | 0.6 | 0.5483871 | 0.6 | 0.46153846 | 0.68 | colon.bma.model.cluster.Goblet.cell.RDS |
| −0.00022 | alt.negative.9:117568766, | | | | | | | | | | |
| 0.00901 | additive.9:117568766, | | | | | | | | | | |
| 0.00504 | ref.negative.14:98734567, | | | | | | | | | | |
| 0.00194 | ref.negative.21:45658474 | | | | | | | | | | |
| 0.0129 | ref.negative.20:62305274, | 4 | 0.37254902 | 0.54901961 | 0.55 | 0.57894737 | 0.53125 | 0.57894737 | 0.42307692 | 0.68 | colon.bma.model.cluster.Goblet.cell.RDS |
| 4e−05 | alt.negative.9:117568766, | | | | | | | | | | |
| 0.0095 | additive.9:117568766, | | | | | | | | | | |
| 0.0068 | ref.negative.14:98734567, | | | | | | | | | | |
| 0.00488 | ref.negative.21:45658474 | | | | | | | | | | |
| 0.00884 | ref.negative.5:11562221, | 4 | 0.41176471 | 0.54901961 | 0.55384615 | 0.57142857 | 0.53333333 | 0.57142857 | 0.46153846 | 0.64 | colon.bma.model.cluster.Goblet.cell.RDS |
| 0.01277 | ref.negative.20:62305274, | | | | | | | | | | |
| −0.00093 | alt.negative.9:117568766, | | | | | | | | | | |
| 0.00882 | additive.9:117568766, | | | | | | | | | | |
| 0.00174 | ref.negative.21:45658474 | | | | | | | | | | |
| 0.01016 | ref.negative.5:11562221, | 4 | 0.47058824 | 0.49019608 | 0.53923077 | 0.5 | 0.48148148 | 0.5 | 0.46153846 | 0.52 | colon.bma.model.cluster.Goblet.cell.RDS |
| 0.01264 | ref.negative.20:62305274, | | | | | | | | | | |
| −0.00303 | alt.negative.9:117568766, | | | | | | | | | | |
| 0.01022 | additive.9:117568766, | | | | | | | | | | |
| 0.00412 | ref.negative.14:98734567 | | | | | | | | | | |
| −0.00095 | alt.negative.9:117568766, | 3 | 0.39215686 | 0.56862745 | 0.54538462 | 0.6 | 0.5483871 | 0.6 | 0.46153846 | 0.68 | colon.bma.model.cluster.Goblet.cell.RDS |
| 0.00922 | additive.9:117568766, | | | | | | | | | | |
| 0.00393 | ref.negative.14:98734567, | | | | | | | | | | |
| 0.00406 | ref.negative.21:45658474 | | | | | | | | | | |
| 0.01087 | ref.negative.20:62305274, | 3 | 0.7254902 | 0.62745098 | 0.62846154 | 0.59459459 | 0.71428571 | 0.59459459 | 0.84615385 | 0.4 | colon.bma.model.cluster.Goblet.cell.RDS |
| 0.00441 | ref.negative.14:98734567, | | | | | | | | | | |
| 0.00931 | ref.negative.21:45658474 | | | | | | | | | | |
| 0.00881 | ref.negative.5:11562221, | 3 | 0.43137255 | 0.56862745 | 0.54538462 | 0.59090909 | 0.55172414 | 0.59090909 | 0.5 | 0.64 | colon.bma.model.cluster.Goblet.cell.RDS |
| −0.00211 | alt.negative.9:117568766, | | | | | | | | | | |
| 0.00961 | additive.9:117568766, | | | | | | | | | | |
| 0.00292 | ref.negative.21:45658474 | | | | | | | | | | |
| 0.01129 | ref.negative.5:11562221, | 3 | 0.76470588 | 0.62745098 | 0.62538462 | 0.58974359 | 0.75 | 0.58974359 | 0.88461538 | 0.36 | colon.bma.model.cluster.Goblet.cell.RDS |
| 0.00944 | ref.negative.20:62305274, | | | | | | | | | | |
| 0.00563 | ref.negative.21:45658474 | | | | | | | | | | |
| 0.01175 | ref.negative.5:11562221, | 3 | 0.78431373 | 0.60784314 | 0.60692308 | 0.575 | 0.72727273 | 0.575 | 0.88461538 | 0.32 | colon.bma.model.cluster.Goblet.cell.RDS |
| 0.00336 | ref.negative.14:98734567, | | | | | | | | | | |
| 0.00692 | ref.negative.21:45658474 | | | | | | | | | | |
| 0.01295 | ref.negative.5:11562221, | 3 | 0.82352941 | 0.60784314 | 0.60307692 | 0.57142857 | 0.77777778 | 0.57142857 | 0.92307692 | 0.28 | colon.bma.model.cluster.Goblet.cell.RDS |
| 0.00924 | ref.negative.20:62305274, | | | | | | | | | | |
| 0.00438 | ref.negative.14:98734567 | | | | | | | | | | |
| 0.0132 | ref.negative.20:62305274, | 3 | 0.41176471 | 0.54901961 | 0.54153846 | 0.57142857 | 0.53333333 | 0.57142857 | 0.46153846 | 0.64 | colon.bma.model.cluster.Goblet.cell.RDS |
| 5e−05 | alt.negative.9:117568766, | | | | | | | | | | |
| 0.00905 | additive.9:117568766, | | | | | | | | | | |
| 0.00431 | ref.negative.21:45658474 | | | | | | | | | | |
| 0.01073 | ref.negative.5:11562221, | 3 | 0.49019608 | 0.50980392 | 0.52923077 | 0.52 | 0.5 | 0.52 | 0.5 | 0.52 | colon.bma.model.cluster.Goblet.cell.RDS |
| −0.00137 | alt.negative.9:117568766, | | | | | | | | | | |
| 0.00957 | additive.9:117568766, | | | | | | | | | | |
| 0.00586 | ref.negative.14:98734567 | | | | | | | | | | |
| 0.00939 | ref.negative.5:11562221, | 3 | 0.50980392 | 0.49019608 | 0.52846154 | 0.5 | 0.48 | 0.5 | 0.5 | 0.48 | colon.bma.model.cluster.Goblet.cell.RDS |
| 0.01369 | ref.negative.20:62305274, | | | | | | | | | | |
| −0.00063 | alt.negative.9:117568766, | | | | | | | | | | |
| 0.00967 | additive.9:117568766 | | | | | | | | | | |
| 0.01173 | ref.negative.20:62305274, | 3 | 0.47058824 | 0.49019608 | 0.52769231 | 0.5 | 0.48148148 | 0.5 | 0.46153846 | 0.52 | colon.bma.model.cluster.Goblet.cell.RDS |
| −0.00167 | alt.negative.9:117568766, | | | | | | | | | | |
| 0.00888 | additive.9:117568766, | | | | | | | | | | |
| 0.00485 | ref.negative.14:98734567 | | | | | | | | | | |
| 0.01038 | haploinsuff.11:128443099, | 7 | 0.31372549 | 0.52941176 | 0.56846154 | 0.5625 | 0.51428571 | 0.5625 | 0.34615385 | 0.72 | colon.bma.model.cluster.moDC.RDS |
| 0.00307 | alt.negative.11:128443099, | | | | | | | | | | |
| 0 | ref.negative.11:128443099, | | | | | | | | | | |
| −0.00107 | additive.16:6147645, | | | | | | | | | | |
| 0.01253 | alt.recessive.16:6147645, | | | | | | | | | | |
| 0 | alt.negative.16:6147645, | | | | | | | | | | |
| 0.00949 | ref.negative.5:11562221, | | | | | | | | | | |
| 0.00461 | ref.negative.14:98734567, | | | | | | | | | | |
| 0.00156 | ref.negative.20:62305274, | | | | | | | | | | |
| −9e−05 | alt.negative.9:117568766, | | | | | | | | | | |
| −0.00865 | ref.negative.21:45658474 | | | | | | | | | | |
| 0.01143 | haploinsuff.11:128443099, | 6 | 0.41176471 | 0.58823529 | 0.61230769 | 0.61904762 | 0.56666667 | 0.61904762 | 0.5 | 0.68 | colon.bma.model.cluster.moDC.RDS |
| 0.00342 | alt.negative.11:128443099, | | | | | | | | | | |
| 0 | ref.negative.11:128443099, | | | | | | | | | | |
| −0.00129 | additive.16:6147645, | | | | | | | | | | |
| 0.01373 | alt.recessive.16:6147645, | | | | | | | | | | |
| 0 | alt.negative.16:6147645, | | | | | | | | | | |
| 0.00855 | ref.negative.5:11562221, | | | | | | | | | | |
| 0.00622 | ref.negative.14:98734567, | | | | | | | | | | |
| 0.00235 | ref.negative.20:62305274, | | | | | | | | | | |
| −0.00209 | alt.negative.9:117568766 | | | | | | | | | | |
| 0.01053 | haploinsuff.11:128443099, | 6 | 0.31372549 | 0.52941176 | 0.56307692 | 0.5625 | 0.51428571 | 0.5625 | 0.34615385 | 0.72 | colon.bma.model.cluster.moDC.RDS |
| 0.00367 | alt.negative.11:128443099, | | | | | | | | | | |
| 0 | ref.negative.11:128443099, | | | | | | | | | | |
| −0.00255 | additive.16:6147645, | | | | | | | | | | |
| 0.01557 | alt.recessive.16:6147645, | | | | | | | | | | |
| 0 | alt.negative.16:6147645, | | | | | | | | | | |
| 0.00909 | ref.negative.5:11562221, | | | | | | | | | | |
| 0.0058 | ref.negative.14:98734567, | | | | | | | | | | |
| 0.00237 | ref.negative.20:62305274, | | | | | | | | | | |
| −0.00796 | ref.negative.21:45658474 | | | | | | | | | | |
| 0.01133 | haploinsuff.11:128443099, | 6 | 0.31372549 | 0.52941176 | 0.56230769 | 0.5625 | 0.51428571 | 0.5625 | 0.34615385 | 0.72 | colon.bma.model.cluster.moDC.RDS |
| 0.00331 | alt.negative.11:128443099, | | | | | | | | | | |
| 0 | ref.negative.11:128443099, | | | | | | | | | | |
| −0.00188 | additive.16:6147645, | | | | | | | | | | |
| 0.01447 | alt.recessive.16:6147645, | | | | | | | | | | |
| 0 | alt.negative.16:6147645, | | | | | | | | | | |
| 0.0102 | ref.negative.5:11562221, | | | | | | | | | | |
| 0.00589 | ref.negative.14:98734567, | | | | | | | | | | |
| −0.00204 | alt.negative.9:117568766, | | | | | | | | | | |
| −0.00815 | ref.negative.21:45658474 | | | | | | | | | | |
| 0.01044 | haploinsuff.11:128443099, | 6 | 0.31372549 | 0.52941176 | 0.56769231 | 0.5625 | 0.51428571 | 0.5625 | 0.34615385 | 0.72 | colon.bma.model.cluster.moDC.RDS |
| 0.00323 | alt.negative.11:128443099, | | | | | | | | | | |
| 0 | ref.negative.11:128443099, | | | | | | | | | | |
| −0.00081 | additive.16:6147645, | | | | | | | | | | |
| 0.01173 | alt.recessive.16:6147645, | | | | | | | | | | |
| 0 | alt.negative.16:6147645, | | | | | | | | | | |
| 0.00506 | ref.negative.14:98734567, | | | | | | | | | | |
| 0.00195 | ref.negative.20:62305274, | | | | | | | | | | |
| −0.00119 | alt.negative.9:117568766, | | | | | | | | | | |
| −0.00717 | ref.negative.21:45658474 | | | | | | | | | | |
| 0.00063 | additive.16:6147645, | 6 | 0.47058824 | 0.52941176 | 0.53461538 | 0.54166667 | 0.51851852 | 0.54166667 | 0.5 | 0.56 | colon.bma.model.cluster.moDC.RDS |
| 0.01075 | alt.recessive.16:6147645, | | | | | | | | | | |
| 0 | alt.negative.16:6147645, | | | | | | | | | | |
| 0.01026 | ref.negative.5:11562221, | | | | | | | | | | |
| 0.00308 | ref.negative.14:98734567, | | | | | | | | | | |
| 0.00453 | ref.negative.20:62305274, | | | | | | | | | | |
| 0.00065 | alt.negative.9:117568766, | | | | | | | | | | |
| −0.0121 | ref.negative.21:45658474 | | | | | | | | | | |
| 0.0105 | haploinsuff.11:128443099, | 6 | 0.33333333 | 0.50980392 | 0.55230769 | 0.52941176 | 0.5 | 0.52941176 | 0.34615385 | 0.68 | colon.bma.model.cluster.moDC.RDS |
| 0.00336 | alt.negative.11:128443099, | | | | | | | | | | |
| 0 | ref.negative.11:128443099, | | | | | | | | | | |
| −0.00185 | additive.16:6147645, | | | | | | | | | | |
| 0.0153 | alt.recessive.16:6147645, | | | | | | | | | | |
| 0 | alt.negative.16:6147645, | | | | | | | | | | |
| 0.00991 | ref.negative.5:11562221, | | | | | | | | | | |
| 0.00196 | ref.negative.20:62305274, | | | | | | | | | | |
| −0.00127 | alt.negative.9:117568766, | | | | | | | | | | |
| −0.00866 | ref.negative.21:45658474 | | | | | | | | | | |
| 0.00997 | haploinsuff.11:128443099, | 6 | 0.45098039 | 0.50980392 | 0.53461538 | 0.52173913 | 0.5 | 0.52173913 | 0.46153846 | 0.56 | colon.bma.model.cluster.moDC.RDS |
| 0.00315 | alt.negative.11:128443099, | | | | | | | | | | |
| 0 | ref.negative.11:128443099, | | | | | | | | | | |
| 0.01062 | ref.negative.5:11562221, | | | | | | | | | | |
| 0.00853 | ref.negative.14:98734567, | | | | | | | | | | |
| 0.00133 | ref.negative.20:62305274, | | | | | | | | | | |
| 0.00245 | alt.negative.9:117568766, | | | | | | | | | | |
| −0.01076 | ref.negative.21:45658474 | | | | | | | | | | |
| 0.0105 | haploinsuff.11:128443099, | 5 | 0.41176471 | 0.58823529 | 0.61461538 | 0.61904762 | 0.56666667 | 0.61904762 | 0.5 | 0.68 | colon.bma.model.cluster.moDC.RDS |
| 0.00263 | alt.negative.11:128443099, | | | | | | | | | | |
| 0 | ref.negative.11:128443099, | | | | | | | | | | |
| −0.00167 | additive.16:6147645, | | | | | | | | | | |
| 0.01262 | alt.recessive.16:6147645, | | | | | | | | | | |
| 0 | alt.negative.16:6147645, | | | | | | | | | | |
| 0.00632 | ref.negative.5:11562221, | | | | | | | | | | |
| 0.00625 | ref.negative.14:98734567, | | | | | | | | | | |
| 0.00147 | ref.negative.20:62305274 | | | | | | | | | | |
| 0.00038 | additive.16:6147645, | 5 | 0.41176471 | 0.58823529 | 0.62384615 | 0.61904762 | 0.56666667 | 0.61904762 | 0.5 | 0.68 | colon.bma.model.cluster.moDC.RDS |
| 0.01091 | alt.recessive.16:6147645, | | | | | | | | | | |
| 0 | alt.negative.16:6147645, | | | | | | | | | | |
| 0.00676 | ref.negative.5:11562221, | | | | | | | | | | |
| 0.00312 | ref.negative.14:98734567, | | | | | | | | | | |
| 0.00426 | ref.negative.20:62305274, | | | | | | | | | | |
| −0.00194 | alt.negative.9:117568766 | | | | | | | | | | |
| 0.01116 | haploinsuff.11:128443099, | 5 | 0.35294118 | 0.56862745 | 0.60846154 | 0.61111111 | 0.54545455 | 0.61111111 | 0.42307692 | 0.72 | colon.bma.model.cluster.moDC.RDS |
| 0.0028 | alt.negative.11:128443099, | | | | | | | | | | |
| 0 | ref.negative.11:128443099, | | | | | | | | | | |
| −0.00033 | additive.16:6147645, | | | | | | | | | | |
| 0.01231 | alt.recessive.16:6147645, | | | | | | | | | | |
| 0 | alt.negative.16:6147645, | | | | | | | | | | |
| 0.00631 | ref.negative.14:98734567, | | | | | | | | | | |
| 0.00147 | ref.negative.20:62305274, | | | | | | | | | | |
| 1e−04 | alt.negative.9:117568766 | | | | | | | | | | |
| 0.0095 | haploinsuff.11:128443099, | 5 | 0.39215686 | 0.56862745 | 0.60384615 | 0.6 | 0.5483871 | 0.6 | 0.46153846 | 0.68 | colon.bma.model.cluster.moDC.RDS |
| 0.00286 | alt.negative.11:128443099, | | | | | | | | | | |
| 0 | ref.negative.11:128443099, | | | | | | | | | | |
| −0.00036 | additive.16:6147645, | | | | | | | | | | |
| 0.0108 | alt.recessive.16:6147645, | | | | | | | | | | |
| 0 | alt.negative.16:6147645, | | | | | | | | | | |
| 0.00721 | ref.negative.5:11562221, | | | | | | | | | | |
| 0.00538 | ref.negative.14:98734567, | | | | | | | | | | |
| −0.00075 | alt.negative.9:117568766 | | | | | | | | | | |
| 0.01051 | haploinsuff.11:128443099, | 5 | 0.39215686 | 0.56862745 | 0.57692308 | 0.6 | 0.5483871 | 0.6 | 0.46153846 | 0.68 | colon.bma.model.cluster.moDC.RDS |
| 0.00366 | alt.negative.11:128443099, | | | | | | | | | | |
| 0 | ref.negative.11:128443099, | | | | | | | | | | |
| −0.00211 | additive.16:6147645, | | | | | | | | | | |
| 0.01526 | alt.recessive.16:6147645, | | | | | | | | | | |
| 0 | alt.negative.16:6147645, | | | | | | | | | | |
| 0.00869 | ref.negative.5:11562221, | | | | | | | | | | |
| 0.0022 | ref.negative.20:62305274, | | | | | | | | | | |
| −0.00388 | alt.negative.9:117568766 | | | | | | | | | | |
| −0.00047 | additive.16:6147645, | 5 | 0.43137255 | 0.56862745 | 0.57461538 | 0.59090909 | 0.55172414 | 0.59090909 | 0.5 | 0.64 | colon.bma.model.cluster.moDC.RDS |
| 0.01192 | alt.recessive.16:6147645, | | | | | | | | | | |
| 0 | alt.negative.16:6147645, | | | | | | | | | | |
| 0.01042 | ref.negative.5:11562221, | | | | | | | | | | |
| 0.00248 | ref.negative.14:98734567, | | | | | | | | | | |
| 0.00425 | ref.negative.20:62305274, | | | | | | | | | | |
| −0.01038 | ref.negative.21:45658474 | | | | | | | | | | |
| 0.00094 | additive.16:6147645, | 5 | 0.43137255 | 0.56862745 | 0.55923077 | 0.59090909 | 0.55172414 | 0.59090909 | 0.5 | 0.64 | colon.bma.model.cluster.moDC.RDS |
| 0.01044 | alt.recessive.16:6147645, | | | | | | | | | | |
| 0 | alt.negative.16:6147645, | | | | | | | | | | |
| 0.00366 | ref.negative.14:98734567, | | | | | | | | | | |
| 0.00426 | ref.negative.20:62305274, | | | | | | | | | | |
| 0.00149 | alt.negative.9:117568766, | | | | | | | | | | |
| −0.00532 | ref.negative.21:45658474 | | | | | | | | | | |
| 0.01003 | ref.negative.5:11562221, | 5 | 0.82352941 | 0.60784314 | 0.56538462 | 0.57142857 | 0.77777778 | 0.57142857 | 0.92307692 | 0.28 | colon.bma.model.cluster.moDC.RDS |
| 0.00621 | ref.negative.14:98734567, | | | | | | | | | | |
| 0.00348 | ref.negative.20:62305274, | | | | | | | | | | |
| −2e−05 | alt.negative.9:117568766, | | | | | | | | | | |
| −0.0059 | ref.negative.21:45658474 | | | | | | | | | | |
| 0.0099 | haploinsuff.11:128443099, | 5 | 0.31372549 | 0.52941176 | 0.55076923 | 0.5625 | 0.51428571 | 0.5625 | 0.34615385 | 0.72 | colon.bma.model.cluster.moDC.RDS |
| 0.0031 | alt.negative.11:128443099, | | | | | | | | | | |
| 0 | ref.negative.11:128443099, | | | | | | | | | | |
| −0.00147 | additive.16:6147645, | | | | | | | | | | |
| 0.01285 | alt.recessive.16:6147645, | | | | | | | | | | |
| 0 | alt.negative.16:6147645, | | | | | | | | | | |
| 0.01016 | ref.negative.5:11562221, | | | | | | | | | | |
| 0.00585 | ref.negative.14:98734567, | | | | | | | | | | |
| −0.00761 | ref.negative.21:45658474 | | | | | | | | | | |
| 0.01015 | haploinsuff.11:128443099, | 5 | 0.31372549 | 0.52941176 | 0.56230769 | 0.5625 | 0.51428571 | 0.5625 | 0.34615385 | 0.72 | colon.bma.model.cluster.moDC.RDS |
| 0.00355 | alt.negative.11:128443099, | | | | | | | | | | |
| 0 | ref.negative.11:128443099, | | | | | | | | | | |
| −0.00048 | additive.16:6147645, | | | | | | | | | | |
| 0.01162 | alt.recessive.16:6147645, | | | | | | | | | | |
| 0 | alt.negative.16:6147645, | | | | | | | | | | |
| 0.00619 | ref.negative.14:98734567, | | | | | | | | | | |
| 0.0018 | ref.negative.20:62305274, | | | | | | | | | | |
| −0.00859 | ref.negative.21:45658474 | | | | | | | | | | |
| 0.01063 | haploinsuff.11:128443099, | 5 | 0.31372549 | 0.52941176 | 0.57076923 | 0.5625 | 0.51428571 | 0.5625 | 0.34615385 | 0.72 | colon.bma.model.cluster.moDC.RDS |
| 0.00268 | alt.negative.11:128443099, | | | | | | | | | | |
| 0 | ref.negative.11:128443099, | | | | | | | | | | |
| −0.00078 | additive.16:6147645, | | | | | | | | | | |
| 0.01174 | alt.recessive.16:6147645, | | | | | | | | | | |
| 0 | alt.negative.16:6147645, | | | | | | | | | | |
| 0.00601 | ref.negative.14:98734567, | | | | | | | | | | |
| 5e−04 | alt.negative.9:117568766, | | | | | | | | | | |
| −0.00565 | ref.negative.21:45658474 | | | | | | | | | | |
| 0.00026 | additive.16:6147645, | 5 | 0.49019608 | 0.54901961 | 0.53 | 0.56 | 0.53846154 | 0.56 | 0.53846154 | 0.56 | colon.bma.model.cluster.moDC.RDS |
| 0.01006 | alt.recessive.16:6147645, | | | | | | | | | | |
| 0 | alt.negative.16:6147645, | | | | | | | | | | |
| 0.00967 | ref.negative.5:11562221, | | | | | | | | | | |
| 0.00404 | ref.negative.20:62305274, | | | | | | | | | | |
| −0.00048 | alt.negative.9:117568766, | | | | | | | | | | |
| −0.01159 | ref.negative.21:45658474 | | | | | | | | | | |
| 0.01012 | haploinsuff.11:128443099, | 5 | 0.47058824 | 0.52941176 | 0.51769231 | 0.54166667 | 0.51851852 | 0.54166667 | 0.5 | 0.56 | colon.bma.model.cluster.moDC.RDS |
| 0.00328 | alt.negative.11:128443099, | | | | | | | | | | |
| 0 | ref.negative.11:128443099, | | | | | | | | | | |
| 0.01019 | ref.negative.5:11562221, | | | | | | | | | | |
| 0.00948 | ref.negative.14:98734567, | | | | | | | | | | |
| −0.00145 | alt.negative.9:117568766, | | | | | | | | | | |
| −0.0081 | ref.negative.21:45658474 | | | | | | | | | | |
| 0.00971 | haploinsuff.11:128443099, | 5 | 0.33333333 | 0.50980392 | 0.55230769 | 0.52941176 | 0.5 | 0.52941176 | 0.34615385 | 0.68 | colon.bma.model.cluster.moDC.RDS |
| 0.00363 | alt.negative.11:128443099, | | | | | | | | | | |
| 0 | ref.negative.11:128443099, | | | | | | | | | | |
| −0.00221 | additive.16:6147645, | | | | | | | | | | |
| 0.01457 | altrecessive.16:6147645, | | | | | | | | | | |
| 0 | alt.negative.16:6147645, | | | | | | | | | | |
| 0.00937 | ref.negative.5:11562221, | | | | | | | | | | |
| 0.00199 | ref.negative.20:62305274, | | | | | | | | | | |
| −0.00958 | ref.negative.21:45658474 | | | | | | | | | | |
| 0.01097 | haploinsuff.11:128443099, | 5 | 0.33333333 | 0.50980392 | 0.55384615 | 0.52941176 | 0.5 | 0.52941176 | 0.34615385 | 0.68 | colon.bma.model.cluster.moDC.RDS |
| 0.00293 | alt.negative.11:128443099, | | | | | | | | | | |
| 0 | ref.negative.11:128443099, | | | | | | | | | | |
| −0.00139 | additive.16:6147645, | | | | | | | | | | |
| 0.013 | alt.recessive.16:6147645, | | | | | | | | | | |
| 0 | alt.negative.16:6147645, | | | | | | | | | | |
| 0.00925 | ref.negative.5:11562221, | | | | | | | | | | |
| −0.00133 | alt.negative.9:117568766, | | | | | | | | | | |
| −0.00554 | ref.negative.21:45658474 | | | | | | | | | | |
| 0.00927 | haploinsuff.11:128443099, | 5 | 0.33333333 | 0.50980392 | 0.54846154 | 0.52941176 | 0.5 | 0.52941176 | 0.34615385 | 0.68 | colon.bma.model.cluster.moDC.RDS |
| 0.00399 | alt.negative.11:128443099, | | | | | | | | | | |
| 0 | ref.negative.11:128443099, | | | | | | | | | | |
| −0.00039 | additive.16:6147645, | | | | | | | | | | |
| 0.01222 | alt.recessive.16:6147645, | | | | | | | | | | |
| 0 | alt.negative.16:6147645, | | | | | | | | | | |
| 0.00223 | ref.negative.20:62305274, | | | | | | | | | | |
| −0.00073 | alt.negative.9:117568766, | | | | | | | | | | |
| −0.00806 | ref.negative.21:45658474 | | | | | | | | | | |
| 0.00955 | haploinsuff.11:128443099, | 5 | 0.45098039 | 0.50980392 | 0.53076923 | 0.52173913 | 0.5 | 0.52173913 | 0.46153846 | 0.56 | colon.bma.model.cluster.moDC.RDS |
| 0.00292 | alt.negative.11:128443099, | | | | | | | | | | |
| 0 | ref.negative.11:128443099, | | | | | | | | | | |
| 0.00832 | ref.negative.5:11562221, | | | | | | | | | | |
| 0.00857 | ref.negative.14:98734567, | | | | | | | | | | |
| 0.00026 | ref.negative.20:62305274, | | | | | | | | | | |
| −0.00661 | ref.negative.21:45658474 | | | | | | | | | | |
| 3e−05 | additive.16:6147645, | 5 | 0.49019608 | 0.50980392 | 0.53076923 | 0.52 | 0.5 | 0.52 | 0.5 | 0.52 | colon.bma.model.cluster.moDC.RDS |
| 0.01128 | alt.recessive.16:6147645, | | | | | | | | | | |
| 0 | alt.negative.16:6147645, | | | | | | | | | | |
| 0.01182 | ref.negative.5:11562221, | | | | | | | | | | |
| 0.00279 | ref.negative.14:98734567, | | | | | | | | | | |
| −0.00118 | alt.negative.9:117568766, | | | | | | | | | | |
| −0.01152 | ref.negative.21:45658474 | | | | | | | | | | |
| 0.01167 | haploinsuff.11:128443099, | 5 | 0.43137255 | 0.49019608 | 0.55692308 | 0.5 | 0.48275862 | 0.5 | 0.42307692 | 0.56 | colon.bma.model.cluster.moDC.RDS |
| 0.00271 | alt.negative.11:128443099, | | | | | | | | | | |
| 0 | ref.negative.11:128443099, | | | | | | | | | | |
| 0.00903 | ref.negative.5:11562221, | | | | | | | | | | |
| 0.00998 | ref.negative.14:98734567, | | | | | | | | | | |
| 7e−05 | ref.negative.20:62305274, | | | | | | | | | | |
| 0.00026 | alt.negative.9:117568766 | | | | | | | | | | |
| 0.01034 | haploinsuff.11:128443099, | 5 | 0.47058824 | 0.49019608 | 0.49692308 | 0.5 | 0.48148148 | 0.5 | 0.46153846 | 0.52 | colon.bma.model.cluster.moDC.RDS |
| 0.00279 | alt.negative.11:128443099, | | | | | | | | | | |
| 0 | ref.negative.11:128443099, | | | | | | | | | | |
| 0.00943 | ref.negative.5:11562221, | | | | | | | | | | |
| 0.00083 | ref.negative.20:62305274, | | | | | | | | | | |
| 0.00154 | alt.negative.9:117568766, | | | | | | | | | | |
| −0.00718 | ref.negative.21:45658474 | | | | | | | | | | |
| 0.0098 | haploinsuff.11:128443099, | 5 | 0.43137255 | 0.49019608 | 0.52615385 | 0.5 | 0.48275862 | 0.5 | 0.42307692 | 0.56 | colon.bma.model.cluster.moDC.RDS |
| 0.00225 | alt.negative.11:128443099, | | | | | | | | | | |
| 0 | ref.negative.11:128443099, | | | | | | | | | | |
| 0.00903 | ref.negative.14:98734567, | | | | | | | | | | |
| 0.00108 | ref.negative.20:62305274, | | | | | | | | | | |
| 0.003 | alt.negative.9:117568766, | | | | | | | | | | |
| −0.00479 | ref.negative.21:45658474 | | | | | | | | | | |
| 0.01073 | haploinsuff.11:128443099, | 4 | 0.31372549 | 0.56862745 | 0.60615385 | 0.625 | 0.54285714 | 0.625 | 0.38461538 | 0.76 | colon.bma.model.cluster.moDC.RDS |
| 0.00387 | alt.negative.11:128443099, | | | | | | | | | | |
| 0 | ref.negative.11:128443099, | | | | | | | | | | |
| −0.00227 | additive.16:6147645, | | | | | | | | | | |
| 0.01407 | alt.recessive.16:6147645, | | | | | | | | | | |
| 0 | alt.negative.16:6147645, | | | | | | | | | | |
| 0.00839 | ref.negative.5:11562221, | | | | | | | | | | |
| 0.00538 | ref.negative.14:98734567 | | | | | | | | | | |
| 0.01113 | haploinsuff.11:128443099, | 4 | 0.31372549 | 0.56862745 | 0.60615385 | 0.625 | 0.54285714 | 0.625 | 0.38461538 | 0.76 | colon.bma.model.cluster.moDC.RDS |
| 0.00292 | alt.negative.11:128443099, | | | | | | | | | | |
| 0 | ref.negative.11:128443099, | | | | | | | | | | |
| −0.00105 | additive.16:6147645, | | | | | | | | | | |
| 0.01293 | alt.recessive.16:6147645, | | | | | | | | | | |
| 0 | alt.negative.16:6147645, | | | | | | | | | | |
| 0.00587 | ref.negative.14:98734567, | | | | | | | | | | |
| −0.00133 | alt.negative.9:117568766 | | | | | | | | | | |
| 0.00106 | additive.16:6147645, | 4 | 0.41176471 | 0.58823529 | 0.61076923 | 0.61904762 | 0.56666667 | 0.61904762 | 0.5 | 0.68 | colon.bma.model.cluster.moDC.RDS |
| 0.01024 | alt.recessive.16:6147645, | | | | | | | | | | |
| 0 | alt.negative.16:6147645, | | | | | | | | | | |
| 0.00779 | ref.negative.5:11562221, | | | | | | | | | | |
| 0.00347 | ref.negative.14:98734567, | | | | | | | | | | |
| 0.00532 | ref.negative.20:62305274 | | | | | | | | | | |
| 0.00109 | additive.16:6147645, | 4 | 0.41176471 | 0.58823529 | 0.59923077 | 0.61904762 | 0.56666667 | 0.61904762 | 0.5 | 0.68 | colon.bma.model.cluster.moDC.RDS |
| 0.01026 | alt.recessive.16:6147645, | | | | | | | | | | |
| 0 | alt.negative.16:6147645, | | | | | | | | | | |
| 0.00357 | ref.negative.14:98734567, | | | | | | | | | | |
| 0.005 | ref.negative.20:62305274, | | | | | | | | | | |
| −0.00099 | alt.negative.9:117568766 | | | | | | | | | | |
| 0.01044 | haploinsuff.11:128443099, | 4 | 0.35294118 | 0.56862745 | 0.60769231 | 0.61111111 | 0.54545455 | 0.61111111 | 0.42307692 | 0.72 | colon.bma.model.cluster.moDC.RDS |
| 0.00353 | alt.negative.11:128443099, | | | | | | | | | | |
| 0 | ref.negative.11:128443099, | | | | | | | | | | |
| −0.00052 | additive.16:6147645, | | | | | | | | | | |
| 0.01277 | alt.recessive.16:6147645, | | | | | | | | | | |
| 0 | alt.negative.16:6147645, | | | | | | | | | | |
| 0.00648 | ref.negative.14:98734567, | | | | | | | | | | |
| 0.00272 | ref.negative.20:62305274 | | | | | | | | | | |
| 0.01188 | haploinsuff.11:128443099, | 4 | 0.35294118 | 0.56862745 | 0.58076923 | 0.61111111 | 0.54545455 | 0.61111111 | 0.42307692 | 0.72 | colon.bma.model.cluster.moDC.RDS |
| 0.00348 | alt.negative.11:128443099, | | | | | | | | | | |
| 0 | ref.negative.11:128443099, | | | | | | | | | | |
| −0.00146 | additive.16:6147645, | | | | | | | | | | |
| 0.0132 | alt.recessive.16:6147645, | | | | | | | | | | |
| 0 | alt.negative.16:6147645, | | | | | | | | | | |
| 0.00581 | ref.negative.14:98734567, | | | | | | | | | | |
| −0.00393 | ref.negative.21:45658474 | | | | | | | | | | |
| 0.00062 | additive.16:6147645, | 4 | 0.45098039 | 0.58823529 | 0.57615385 | 0.60869565 | 0.57142857 | 0.60869565 | 0.53846154 | 0.64 | colon.bma.model.cluster.moDC.RDS |
| 0.01233 | alt.recessive.16:6147645, | | | | | | | | | | |
| 0 | alt.negative.16:6147645, | | | | | | | | | | |
| 0.00883 | ref.negative.5:11562221, | | | | | | | | | | |
| 0.00617 | ref.negative.20:62305274, | | | | | | | | | | |
| −0.00047 | alt.negative.9:117568766 | | | | | | | | | | |
| 0.00086 | additive.16:6147645, | 4 | 0.45098039 | 0.58823529 | 0.55461538 | 0.60869565 | 0.57142857 | 0.60869565 | 0.53846154 | 0.64 | colon.bma.model.cluster.moDC.RDS |
| 0.01032 | alt.recessive.16:6147645, | | | | | | | | | | |
| 0 | alt.negative.16:6147645, | | | | | | | | | | |
| 0.00899 | ref.negative.5:11562221, | | | | | | | | | | |
| 0.00428 | ref.negative.20:62305274, | | | | | | | | | | |
| −0.00599 | ref.negative.21:45658474 | | | | | | | | | | |
| 0.00104 | additive.16:6147645, | 4 | 0.45098039 | 0.58823529 | 0.54230769 | 0.60869565 | 0.57142857 | 0.60869565 | 0.53846154 | 0.64 | colon.bma.model.cluster.moDC.RDS |
| 0.01084 | alt.recessive.16:6147645, | | | | | | | | | | |
| 0 | alt.negative.16:6147645, | | | | | | | | | | |
| 0.00389 | ref.negative.20:62305274, | | | | | | | | | | |
| 0.00166 | alt.negative.9:117568766, | | | | | | | | | | |
| −0.00772 | ref.negative.21:45658474 | | | | | | | | | | |
| 0.0107 | haploinsuff.11:128443099, | 4 | 0.39215686 | 0.56862745 | 0.58076923 | 0.6 | 0.5483871 | 0.6 | 0.46153846 | 0.68 | colon.bma.model.cluster.moDC.RDS |
| 0.00298 | alt.negative.11:128443099, | | | | | | | | | | |
| 0 | ref.negative.11:128443099, | | | | | | | | | | |
| −0.00114 | additive.16:6147645, | | | | | | | | | | |
| 0.0131 | alt.recessive.16:6147645, | | | | | | | | | | |
| 0 | alt.negative.16:6147645, | | | | | | | | | | |
| 0.00109 | ref.negative.20:62305274, | | | | | | | | | | |
| −0.00174 | alt.negative.9:117568766 | | | | | | | | | | |
| 0.01098 | haploinsuff.11:128443099, | 4 | 0.43137255 | 0.56862745 | 0.58692308 | 0.59090909 | 0.55172414 | 0.59090909 | 0.5 | 0.64 | colon.bma.model.cluster.moDC.RDS |
| 0.00263 | alt.negative.11:128443099, | | | | | | | | | | |
| 0 | ref.negative.11:128443099, | | | | | | | | | | |
| −0.00149 | additive.16:6147645, | | | | | | | | | | |
| 0.01259 | alt.recessive.16:6147645, | | | | | | | | | | |
| 0 | alt.negative.16:6147645, | | | | | | | | | | |
| 0.0062 | ref.negative.5:11562221, | | | | | | | | | | |
| 0.00242 | ref.negative.20:62305274 | | | | | | | | | | |
| 5e−04 | additive.16:6147645, | 4 | 0.43137255 | 0.56862745 | 0.60615385 | 0.59090909 | 0.55172414 | 0.59090909 | 0.5 | 0.64 | colon.bma.model.cluster.moDC.RDS |
| 0.01077 | alt.recessive.16:6147645, | | | | | | | | | | |
| 0 | alt.negative.16:6147645, | | | | | | | | | | |
| 0.00766 | ref.negative.5:11562221, | | | | | | | | | | |
| 0.00294 | ref.negative.14:98734567, | | | | | | | | | | |
| −0.00128 | alt.negative.9:117568766 | | | | | | | | | | |
| 0.00055 | additive.16:6147645, | 4 | 0.43137255 | 0.56862745 | 0.56076923 | 0.59090909 | 0.55172414 | 0.59090909 | 0.5 | 0.64 | colon.bma.model.cluster.moDC.RDS |
| 0.01149 | alt.recessive.16:6147645, | | | | | | | | | | |
| 0 | alt.negative.16:6147645, | | | | | | | | | | |
| 0.00245 | ref.negative.14:98734567, | | | | | | | | | | |
| 0.00429 | ref.negative.20:62305274, | | | | | | | | | | |
| −0.00791 | ref.negative.21:45658474 | | | | | | | | | | |
| 0.00045 | additive.16:6147645, | 4 | 0.47058824 | 0.56862745 | 0.54846154 | 0.58333333 | 0.55555556 | 0.58333333 | 0.53846154 | 0.6 | colon.bma.model.cluster.moDC.RDS |
| 0.01096 | alt.recessive.16:6147645, | | | | | | | | | | |
| 0 | alt.negative.16:6147645, | | | | | | | | | | |
| 0.01353 | ref.negative.5:11562221, | | | | | | | | | | |
| −0.00268 | alt.negative.9:117568766, | | | | | | | | | | |
| −0.0088 | ref.negative.21:45658474 | | | | | | | | | | |
| 0.01117 | haploinsuff.11:128443099, | 4 | 0.41176471 | 0.54901961 | 0.57461538 | 0.57142857 | 0.53333333 | 0.57142857 | 0.46153846 | 0.64 | colon.bma.model.cluster.moDC.RDS |
| 0.00286 | alt.negative.11:128443099, | | | | | | | | | | |
| 0 | ref.negative.11:128443099, | | | | | | | | | | |
| −0.00137 | additive.16:6147645, | | | | | | | | | | |
| 0.014 | alt.recessive.16:6147645, | | | | | | | | | | |
| 0 | alt.negative.16:6147645, | | | | | | | | | | |
| 0.00906 | ref.negative.5:11562221, | | | | | | | | | | |
| −0.00134 | alt.negative.9:117568766 | | | | | | | | | | |
| −0.00077 | additive.16:6147645, | 4 | 0.45098039 | 0.54901961 | 0.55769231 | 0.56521739 | 0.53571429 | 0.56521739 | 0.5 | 0.6 | colon.bma.model.cluster.moDC.RDS |
| 0.013 | alt.recessive.16:6147645, | | | | | | | | | | |
| 0 | alt.negative.16:6147645, | | | | | | | | | | |
| 0.01291 | ref.negative.5:11562221, | | | | | | | | | | |
| 0.00242 | ref.negative.14:98734567, | | | | | | | | | | |
| −0.01222 | ref.negative.21:45658474 | | | | | | | | | | |
| 0.00802 | ref.negative.5:11562221, | 4 | 0.80392157 | 0.58823529 | 0.59 | 0.56097561 | 0.7 | 0.56097561 | 0.88461538 | 0.28 | colon.bma.model.cluster.moDC.RDS |
| 0.0069 | ref.negative.14:98734567, | | | | | | | | | | |
| 0.00313 | ref.negative.20:62305274, | | | | | | | | | | |
| 0.00154 | alt.negative.9:117568766 | | | | | | | | | | |
| 0.01054 | ref.negative.5:11562221, | 4 | 0.84313725 | 0.58823529 | 0.52076923 | 0.55813953 | 0.75 | 0.55813953 | 0.92307692 | 0.24 | colon.bma.model.cluster.moDC.RDS |
| 0.00569 | ref.negative.14:98734567, | | | | | | | | | | |
| 0.00341 | ref.negative.20:62305274, | | | | | | | | | | |
| −0.00735 | ref.negative.21:45658474 | | | | | | | | | | |
| 0.0066 | ref.negative.14:98734567, | 4 | 0.82352941 | 0.56862745 | 0.51692308 | 0.54761905 | 0.66666667 | 0.54761905 | 0.88461538 | 0.24 | colon.bma.model.cluster.moDC.RDS |
| 0.00285 | ref.negative.20:62305274, | | | | | | | | | | |
| 0.00439 | alt.negative.9:117568766, | | | | | | | | | | |
| −0.01035 | ref.negative.21:45658474 | | | | | | | | | | |
| 0.01025 | haploinsuff.11:128443099, | 4 | 0.47058824 | 0.52941176 | 0.52615385 | 0.54166667 | 0.51851852 | 0.54166667 | 0.5 | 0.56 | colon.bma.model.cluster.moDC.RDS |
| 0.00274 | alt.negative.11:128443099, | | | | | | | | | | |
| 0 | ref.negative.11:128443099, | | | | | | | | | | |
| 0.00833 | ref.negative.5:11562221, | | | | | | | | | | |
| 0.00846 | ref.negative.14:98734567, | | | | | | | | | | |
| −0.00526 | ref.negative.21:45658474 | | | | | | | | | | |
| 0.01264 | haploinsuff.11:128443099, | 4 | 0.50980392 | 0.52941176 | 0.52307692 | 0.53846154 | 0.52 | 0.53846154 | 0.53846154 | 0.52 | colon.bma.model.cluster.moDC.RDS |
| 0.00154 | alt.negative.11:128443099, | | | | | | | | | | |
| 0 | ref.negative.11:128443099, | | | | | | | | | | |
| 0.00789 | ref.negative.5:11562221, | | | | | | | | | | |
| −0.00016 | ref.negative.20:62305274, | | | | | | | | | | |
| −0.00062 | alt.negative.9:117568766 | | | | | | | | | | |
| 0.01107 | haploinsuff.11:128443099, | 4 | 0.50980392 | 0.52941176 | 0.50230769 | 0.53846154 | 0.52 | 0.53846154 | 0.53846154 | 0.52 | colon.bma.model.cluster.moDC.RDS |
| 0.00206 | alt.negative.11:128443099, | | | | | | | | | | |
| 0 | ref.negative.11:128443099, | | | | | | | | | | |
| 0.00899 | ref.negative.5:11562221, | | | | | | | | | | |
| −0.00159 | alt.negative.9:117568766, | | | | | | | | | | |
| −0.00559 | ref.negative.21:45658474 | | | | | | | | | | |
| 0.00995 | ref.negative.5:11562221, | 4 | 0.88235294 | 0.54901961 | 0.48692308 | 0.53333333 | 0.66666667 | 0.53333333 | 0.92307692 | 0.16 | colon.bma.model.cluster.moDC.RDS |
| 0.00339 | ref.negative.20:62305274, | | | | | | | | | | |
| 0.00123 | alt.negative.9:117568766, | | | | | | | | | | |
| −0.00617 | ref.negative.21:45658474 | | | | | | | | | | |
| 0.01089 | haploinsuff.11:128443099, | 4 | 0.33333333 | 0.50980392 | 0.55384615 | 0.52941176 | 0.5 | 0.52941176 | 0.34615385 | 0.68 | colon.bma.model.cluster.moDC.RDS |
| 0.00245 | alt.negative.11:128443099, | | | | | | | | | | |
| 0 | ref.negative.11:128443099, | | | | | | | | | | |
| −0.00201 | additive.16:6147645, | | | | | | | | | | |
| 0.01395 | alt.recessive.16:6147645, | | | | | | | | | | |
| 0 | alt.negative.16:6147645, | | | | | | | | | | |
| 0.00875 | ref.negative.5:11562221, | | | | | | | | | | |
| −0.00832 | ref.negative.21:45658474 | | | | | | | | | | |
| 0.01003 | haploinsuff.11:128443099, | 4 | 0.33333333 | 0.50980392 | 0.55615385 | 0.52941176 | 0.5 | 0.52941176 | 0.34615385 | 0.68 | colon.bma.model.cluster.moDC.RDS |
| 0.00298 | alt.negative.11:128443099, | | | | | | | | | | |
| 0 | ref.negative.11:128443099, | | | | | | | | | | |
| −0.0021 | additive.16:6147645, | | | | | | | | | | |
| 0.01331 | alt.recessive.16:6147645, | | | | | | | | | | |
| 0 | alt.negative.16:6147645, | | | | | | | | | | |
| 0.00098 | ref.negative.20:62305274, | | | | | | | | | | |
| −0.00485 | ref.negative.21:45658474 | | | | | | | | | | |
| 0.01003 | haploinsuff.11:128443099, | 4 | 0.33333333 | 0.50980392 | 0.54846154 | 0.52941176 | 0.5 | 0.52941176 | 0.34615385 | 0.68 | colon.bma.model.cluster.moDC.RDS |
| 0.00271 | alt.negative.11:128443099, | | | | | | | | | | |
| 0 | ref.negative.11:128443099, | | | | | | | | | | |
| −0.00143 | additive.16:6147645, | | | | | | | | | | |
| 0.01298 | alt.recessive.16:6147645, | | | | | | | | | | |
| 0 | alt.negative.16:6147645, | | | | | | | | | | |
| −0.001 | alt.negative.9:117568766, | | | | | | | | | | |
| −0.007 | ref.negative.21:45658474 | | | | | | | | | | |
| 0.01051 | haploinsuff.11:128443099, | 4 | 0.45098039 | 0.50980392 | 0.56538462 | 0.52173913 | 0.5 | 0.52173913 | 0.46153846 | 0.56 | colon.bma.model.cluster.moDC.RDS |
| 0.00331 | alt.negative.11:128443099, | | | | | | | | | | |
| 0 | ref.negative.11:128443099, | | | | | | | | | | |
| 0.00773 | ref.negative.5:11562221, | | | | | | | | | | |
| 0.00995 | ref.negative.14:98734567, | | | | | | | | | | |
| 0.00039 | ref.negative.20:62305274 | | | | | | | | | | |
| 0.01121 | haploinsuff.11:128443099, | 4 | 0.45098039 | 0.50980392 | 0.54692308 | 0.52173913 | 0.5 | 0.52173913 | 0.46153846 | 0.56 | colon.bma.model.cluster.moDC.RDS |
| 0.00263 | alt.negative.11:128443099, | | | | | | | | | | |
| 0 | ref.negative.11:128443099, | | | | | | | | | | |
| 0.00739 | ref.negative.5:11562221, | | | | | | | | | | |
| 0.00923 | ref.negative.14:98734567, | | | | | | | | | | |
| 0.00181 | alt.negative.9:117568766 | | | | | | | | | | |
| 0.01164 | haploinsuff.11:128443099, | 4 | 0.45098039 | 0.50980392 | 0.54384615 | 0.52173913 | 0.5 | 0.52173913 | 0.46153846 | 0.56 | colon.bma.model.cluster.moDC.RDS |
| 0.00176 | alt.negative.11:128443099, | | | | | | | | | | |
| 0 | ref.negative.11:128443099, | | | | | | | | | | |
| 0.00988 | ref.negative.14:98734567, | | | | | | | | | | |
| 0 | ref.negative.20:62305274, | | | | | | | | | | |
| 0.00071 | alt.negative.9:117568766 | | | | | | | | | | |
| 0.00982 | haploinsuff.11:128443099, | 4 | 0.45098039 | 0.50980392 | 0.53307692 | 0.52173913 | 0.5 | 0.52173913 | 0.46153846 | 0.56 | colon.bma.model.cluster.moDC.RDS |
| 0.00295 | alt.negative.11:128443099, | | | | | | | | | | |
| 0 | ref.negative.11:128443099, | | | | | | | | | | |
| 0.00877 | ref.negative.14:98734567, | | | | | | | | | | |
| 0.0012 | ref.negative.20:62305274, | | | | | | | | | | |
| −0.00549 | ref.negative.21:45658474 | | | | | | | | | | |
| 0.01243 | haploinsuff.11:128443099, | 4 | 0.45098039 | 0.50980392 | 0.51692308 | 0.52173913 | 0.5 | 0.52173913 | 0.46153846 | 0.56 | colon.bma.model.cluster.moDC.RDS |
| 0.00208 | alt.negative.11:128443099, | | | | | | | | | | |
| 0 | ref.negative.11:128443099, | | | | | | | | | | |
| 0.0099 | ref.negative.14:98734567, | | | | | | | | | | |
| 0.00235 | alt.negative.9:117568766, | | | | | | | | | | |
| −0.00819 | ref.negative.21:45658474 | | | | | | | | | | |
| 0.01045 | ref.negative.5:11562221, | 4 | 0.90196078 | 0.52941176 | 0.46769231 | 0.52173913 | 0.6 | 0.52173913 | 0.92307692 | 0.12 | colon.bma.model.cluster.moDC.RDS |
| 0.00642 | ref.negative.14:98734567, | | | | | | | | | | |
| 0.00155 | alt.negative.9:117568766, | | | | | | | | | | |
| −0.00838 | ref.negative.21:45658474 | | | | | | | | | | |
| 0.01071 | haploinsuff.11:128443099, | 4 | 0.49019608 | 0.50980392 | 0.50384615 | 0.52 | 0.5 | 0.52 | 0.5 | 0.52 | colon.bma.model.cluster.moDC.RDS |
| 0.00207 | alt.negative.11:128443099, | | | | | | | | | | |
| 0 | ref.negative.11:128443099, | | | | | | | | | | |
| 0.0086 | ref.negative.5:11562221, | | | | | | | | | | |
| 5e−05 | ref.negative.20:62305274, | | | | | | | | | | |
| −0.00759 | ref.negative.21:45658474 | | | | | | | | | | |
| 0.01114 | haploinsuff.11:128443099, | 4 | 0.49019608 | 0.50980392 | 0.47769231 | 0.52 | 0.5 | 0.52 | 0.5 | 0.52 | colon.bma.model.cluster.moDC.RDS |
| 0.00184 | alt.negative.11:128443099, | | | | | | | | | | |
| 0 | ref.negative.11:128443099, | | | | | | | | | | |
| −6e−04 | ref.negative.20:62305274, | | | | | | | | | | |
| 0.00108 | alt.negative.9:117568766, | | | | | | | | | | |
| −0.00458 | ref.negative.21:45658474 | | | | | | | | | | |
| 0.00096 | additive.16:6147645, | 4 | 0.49019608 | 0.50980392 | 0.50769231 | 0.52 | 0.5 | 0.52 | 0.5 | 0.52 | colon.bma.model.cluster.moDC.RDS |
| 0.01003 | alt.recessive.16:6147645, | | | | | | | | | | |
| 0 | alt.negative.16:6147645, | | | | | | | | | | |
| 0.00265 | ref.negative.14:98734567, | | | | | | | | | | |
| −0.00072 | alt.negative.9:117568766, | | | | | | | | | | |
| −0.01127 | ref.negative.21:45658474 | | | | | | | | | | |
| −0.00012 | additive.16:6147645, | 3 | 0.43137255 | 0.60784314 | 0.59307692 | 0.63636364 | 0.5862069 | 0.63636364 | 0.53846154 | 0.68 | colon.bma.model.cluster.moDC.RDS |
| 0.01133 | alt.recessive.16:6147645, | | | | | | | | | | |
| 0 | alt.negative.16:6147645, | | | | | | | | | | |
| 0.0043 | ref.negative.20:62305274, | | | | | | | | | | |
| 0.001 | alt.negative.9:117568766 | | | | | | | | | | |
| 0.01176 | haploinsuff.11:128443099, | 3 | 0.31372549 | 0.56862745 | 0.58461538 | 0.625 | 0.54285714 | 0.625 | 0.38461538 | 0.76 | colon.bma.model.cluster.moDC.RDS |
| 0.00211 | alt.negative.11:128443099, | | | | | | | | | | |
| 0 | ref.negative.11:128443099, | | | | | | | | | | |
| −0.00315 | additive.16:6147645, | | | | | | | | | | |
| 0.01576 | alt.recessive.16:6147645, | | | | | | | | | | |
| 0 | alt.negative.16:6147645, | | | | | | | | | | |
| 0.00801 | ref.negative.5:11562221 | | | | | | | | | | |
| 0.01266 | haploinsuff.11:128443099, | 3 | 0.31372549 | 0.56862745 | 0.59307692 | 0.625 | 0.54285714 | 0.625 | 0.38461538 | 0.76 | colon.bma.model.cluster.moDC.RDS |
| 0.00208 | alt.negative.11:128443099, | | | | | | | | | | |
| 0 | ref.negative.11:128443099, | | | | | | | | | | |
| −0.002 | additive.16:6147645, | | | | | | | | | | |
| 0.01409 | alt.recessive.16:6147645, | | | | | | | | | | |
| 0 | alt.negative.16:6147645, | | | | | | | | | | |
| 0.00625 | ref.negative.14:98734567 | | | | | | | | | | |
| 0.00019 | additive.16:6147645, | 3 | 0.41176471 | 0.58823529 | 0.59846154 | 0.61904762 | 0.56666667 | 0.61904762 | 0.5 | 0.68 | colon.bma.model.cluster.moDC.RDS |
| 0.01191 | alt.recessive.16:6147645, | | | | | | | | | | |
| 0 | alt.negative.16:6147645, | | | | | | | | | | |
| 0.00282 | ref.negative.14:98734567, | | | | | | | | | | |
| 0.00475 | ref.negative.20:62305274 | | | | | | | | | | |
| −0.00119 | additive.16:6147645, | 3 | 0.45098039 | 0.58823529 | 0.60846154 | 0.60869565 | 0.57142857 | 0.60869565 | 0.53846154 | 0.64 | colon.bma.model.cluster.moDC.RDS |
| 0.01187 | alt.recessive.16:6147645, | | | | | | | | | | |
| 0 | alt.negative.16:6147645, | | | | | | | | | | |
| 0.00832 | ref.negative.5:11562221, | | | | | | | | | | |
| 0.00489 | ref.negative.20:62305274 | | | | | | | | | | |
| 0.00059 | additive.16:6147645, | 3 | 0.45098039 | 0.58823529 | 0.54230769 | 0.60869565 | 0.57142857 | 0.60869565 | 0.53846154 | 0.64 | colon.bma.model.cluster.moDC.RDS |
| 0.01156 | alt.recessive.16:6147645, | | | | | | | | | | |
| 0 | alt.negative.16:6147645, | | | | | | | | | | |
| 0.0047 | ref.negative.20:62305274, | | | | | | | | | | |
| −0.0054 | ref.negative.21:45658474 | | | | | | | | | | |
| 0.01087 | haploinsuff.11:128443099, | 3 | 0.43137255 | 0.56862745 | 0.58615385 | 0.59090909 | 0.55172414 | 0.59090909 | 0.5 | 0.64 | colon.bma.model.cluster.moDC.RDS |
| 0.00305 | alt.negative.11:128443099, | | | | | | | | | | |
| 0 | ref.negative.11:128443099, | | | | | | | | | | |
| −0.00209 | additive.16:6147645, | | | | | | | | | | |
| 0.01484 | alt.recessive.16:6147645, | | | | | | | | | | |
| 0 | alt.negative.16:6147645, | | | | | | | | | | |
| 0.0021 | ref.negative.20:62305274 | | | | | | | | | | |
| −1e−05 | additive.16:6147645, | 3 | 0.43137255 | 0.56862745 | 0.60923077 | 0.59090909 | 0.55172414 | 0.59090909 | 0.5 | 0.64 | colon.bma.model.cluster.moDC.RDS |
| 0.01219 | alt.recessive.16:6147645, | | | | | | | | | | |
| 0 | alt.negative.16:6147645, | | | | | | | | | | |
| 0.00684 | ref.negative.5:11562221, | | | | | | | | | | |
| 0.00244 | ref.negative.14:98734567 | | | | | | | | | | |
| 0.00023 | additive.16:6147645, | 3 | 0.43137255 | 0.56862745 | 0.58 | 0.59090909 | 0.55172414 | 0.59090909 | 0.5 | 0.64 | colon.bma.model.cluster.moDC.RDS |
| 0.01157 | alt.recessive.16:6147645, | | | | | | | | | | |
| 0 | alt.negative.16:6147645, | | | | | | | | | | |
| 0.00293 | ref.negative.14:98734567, | | | | | | | | | | |
| −2e−05 | alt.negative.9:117568766 | | | | | | | | | | |
| 0.01053 | haploinsuff.11:128443099, | 3 | 0.33333333 | 0.54901961 | 0.57846154 | 0.58823529 | 0.52941176 | 0.58823529 | 0.38461538 | 0.72 | colon.bma.model.cluster.moDC.RDS |
| 0.00255 | alt.negative.11:128443099, | | | | | | | | | | |
| 0 | ref.negative.11:128443099, | | | | | | | | | | |
| −0.00122 | additive.16:6147645, | | | | | | | | | | |
| 0.01237 | altrecessive.16:6147645, | | | | | | | | | | |
| 0 | alt.negative.16:6147645, | | | | | | | | | | |
| −0.00273 | alt.negative.9:117568766 | | | | | | | | | | |
| −0.00051 | additive.16:6147645, | 3 | 0.47058824 | 0.56862745 | 0.58538462 | 0.58333333 | 0.55555556 | 0.58333333 | 0.53846154 | 0.6 | colon.bma.model.cluster.moDC.RDS |
| 0.01276 | alt.recessive.16:6147645, | | | | | | | | | | |
| 0 | alt.negative.16:6147645, | | | | | | | | | | |
| 0.00956 | ref.negative.5:11562221, | | | | | | | | | | |
| −0.00124 | alt.negative.9:117568766 | | | | | | | | | | |
| −0.00024 | additive.16:6147645, | 3 | 0.47058824 | 0.56862745 | 0.55230769 | 0.58333333 | 0.55555556 | 0.58333333 | 0.53846154 | 0.6 | colon.bma.model.cluster.moDC.RDS |
| 0.01201 | alt.recessive.16:6147645, | | | | | | | | | | |
| 0 | alt.negative.16:6147645, | | | | | | | | | | |
| 0.00939 | ref.negative.5:11562221, | | | | | | | | | | |
| −0.00904 | ref.negative.21:45658474 | | | | | | | | | | |
| 0.00058 | additive.16:6147645, | 3 | 0.47058824 | 0.56862745 | 0.52076923 | 0.58333333 | 0.55555556 | 0.58333333 | 0.53846154 | 0.6 | colon.bma.model.cluster.moDC.RDS |
| 0.01056 | alt.recessive.16:6147645, | | | | | | | | | | |
| 0 | alt.negative.16:6147645, | | | | | | | | | | |
| 0.00114 | alt.negative.9:117568766, | | | | | | | | | | |
| −0.0053 | ref.negative.21:45658474 | | | | | | | | | | |
| 0.00909 | ref.negative.5:11562221, | 3 | 0.82352941 | 0.60784314 | 0.60307692 | 0.57142857 | 0.77777778 | 0.57142857 | 0.92307692 | 0.28 | colon.bma.model.cluster.moDC.RDS |
| 0.00681 | ref.negative.14:98734567, | | | | | | | | | | |
| 0.0038 | ref.negative.20:62305274 | | | | | | | | | | |
| −0.00022 | additive.16:6147645, | 3 | 0.45098039 | 0.54901961 | 0.55461538 | 0.56521739 | 0.53571429 | 0.56521739 | 0.5 | 0.6 | colon.bma.model.cluster.moDC.RDS |
| 0.01056 | alt.recessive.16:6147645, | | | | | | | | | | |
| 0 | alt.negative.16:6147645, | | | | | | | | | | |
| 0.0027 | ref.negative.14:98734567, | | | | | | | | | | |
| −0.00658 | ref.negative.21:45658474 | | | | | | | | | | |
| 0.00583 | ref.negative.14:98734567, | 3 | 0.84313725 | 0.58823529 | 0.53461538 | 0.55813953 | 0.75 | 0.55813953 | 0.92307692 | 0.24 | colon.bma.model.cluster.moDC.RDS |
| 0.003 | ref.negative.20:62305274, | | | | | | | | | | |
| −0.00574 | ref.negative.21:45658474 | | | | | | | | | | |
| 0.00686 | ref.negative.14:98734567, | 3 | 0.82352941 | 0.56862745 | 0.56769231 | 0.54761905 | 0.66666667 | 0.54761905 | 0.88461538 | 0.24 | colon.bma.model.cluster.moDC.RDS |
| 0.00368 | ref.negative.20:62305274, | | | | | | | | | | |
| 0.00272 | alt.negative.9:117568766 | | | | | | | | | | |
| 0.01013 | ref.negative.5:11562221, | 3 | 0.8627451 | 0.56862745 | 0.56461538 | 0.54545455 | 0.71428571 | 0.54545455 | 0.92307692 | 0.2 | colon.bma.model.cluster.moDC.RDS |
| 0.00397 | ref.negative.20:62305274, | | | | | | | | | | |
| 0.0012 | alt.negative.9:117568766 | | | | | | | | | | |
| 0.00917 | ref.negative.5:11562221, | 3 | 0.90196078 | 0.56862745 | 0.51615385 | 0.54347826 | 0.8 | 0.54347826 | 0.96153846 | 0.16 | colon.bma.model.cluster.moDC.RDS |
| 0.00667 | ref.negative.14:98734567, | | | | | | | | | | |
| −0.00557 | ref.negative.21:45658474 | | | | | | | | | | |
| 0.01059 | ref.negative.5:11562221, | 3 | 0.90196078 | 0.56862745 | 0.49769231 | 0.54347826 | 0.8 | 0.54347826 | 0.96153846 | 0.16 | colon.bma.model.cluster.moDC.RDS |
| 0.0032 | ref.negative.20:62305274, | | | | | | | | | | |
| −0.00783 | ref.negative.21:45658474 | | | | | | | | | | |
| 0.0115 | haploinsuff.11:128443099, | 3 | 0.47058824 | 0.52941176 | 0.55538462 | 0.54166667 | 0.51851852 | 0.54166667 | 0.5 | 0.56 | colon.bma.model.cluster.moDC.RDS |
| 0.00246 | alt.negative.11:128443099, | | | | | | | | | | |
| 0 | ref.negative.11:128443099, | | | | | | | | | | |
| 0.00754 | ref.negative.5:11562221, | | | | | | | | | | |
| 0.01015 | ref.negative.14:98734567 | | | | | | | | | | |
| 0.0121 | haploinsuff.11:128443099, | 3 | 0.47058824 | 0.52941176 | 0.52538462 | 0.54166667 | 0.51851852 | 0.54166667 | 0.5 | 0.56 | colon.bma.model.cluster.moDC.RDS |
| 0.00207 | alt.negative.11:128443099, | | | | | | | | | | |
| 0 | ref.negative.11:128443099, | | | | | | | | | | |
| 0.01046 | ref.negative.14:98734567, | | | | | | | | | | |
| −0.00294 | ref.negative.21:45658474 | | | | | | | | | | |
| 0.01184 | haploinsuff.11:128443099, | 3 | 0.50980392 | 0.52941176 | 0.53230769 | 0.53846154 | 0.52 | 0.53846154 | 0.53846154 | 0.52 | colon.bma.model.cluster.moDC.RDS |
| 0.0025 | alt.negative.11:128443099, | | | | | | | | | | |
| 0 | ref.negative.11:128443099, | | | | | | | | | | |
| 0.00721 | ref.negative.5:11562221, | | | | | | | | | | |
| −0.00097 | alt.negative.9:117568766 | | | | | | | | | | |
| 0.01087 | haploinsuff.11:128443099, | 3 | 0.50980392 | 0.52941176 | 0.49538462 | 0.53846154 | 0.52 | 0.53846154 | 0.53846154 | 0.52 | colon.bma.model.cluster.moDC.RDS |
| 0.00273 | alt.negative.11:128443099, | | | | | | | | | | |
| 0 | ref.negative.11:128443099, | | | | | | | | | | |
| 0.00771 | ref.negative.5:11562221, | | | | | | | | | | |
| −0.00423 | ref.negative.21:45658474 | | | | | | | | | | |
| 0.01032 | haploinsuff.11:128443099, | 3 | 0.50980392 | 0.52941176 | 0.48461538 | 0.53846154 | 0.52 | 0.53846154 | 0.53846154 | 0.52 | colon.bma.model.cluster.moDC.RDS |
| 0.00137 | alt.negative.11:128443099, | | | | | | | | | | |
| 0 | ref.negative.11:128443099, | | | | | | | | | | |
| −9e−05 | ref.negative.20:62305274, | | | | | | | | | | |
| −0.0043 | ref.negative.21:45658474 | | | | | | | | | | |
| 0.00866 | ref.negative.5:11562221, | 3 | 0.88235294 | 0.54901961 | 0.54615385 | 0.53333333 | 0.66666667 | 0.53333333 | 0.92307692 | 0.16 | colon.bma.model.cluster.moDC.RDS |
| 0.00739 | ref.negative.14:98734567, | | | | | | | | | | |
| 0.00056 | alt.negative.9:117568766 | | | | | | | | | | |
| 0.0108 | haploinsuff.11:128443099, | 3 | 0.33333333 | 0.50980392 | 0.54384615 | 0.52941176 | 0.5 | 0.52941176 | 0.34615385 | 0.68 | colon.bma.model.cluster.moDC.RDS |
| 0.00286 | alt.negative.11:128443099, | | | | | | | | | | |
| 0 | ref.negative.11:128443099, | | | | | | | | | | |
| −0.00088 | additive.16:6147645, | | | | | | | | | | |
| 0.01259 | alt.recessive.16:6147645, | | | | | | | | | | |
| 0 | alt.negative.16:6147645, | | | | | | | | | | |
| −0.00595 | ref.negative.21:45658474 | | | | | | | | | | |
| 0.01148 | haploinsuff.11:128443099, | 3 | 0.45098039 | 0.50980392 | 0.56230769 | 0.52173913 | 0.5 | 0.52173913 | 0.46153846 | 0.56 | colon.bma.model.cluster.moDC.RDS |
| 0.00253 | alt.negative.11:128443099, | | | | | | | | | | |
| 0 | ref.negative.11:128443099, | | | | | | | | | | |
| 0.00973 | ref.negative.14:98734567, | | | | | | | | | | |
| 0.00025 | ref.negative.20:62305274 | | | | | | | | | | |
| 0.01242 | haploinsuff.11:128443099, | 3 | 0.45098039 | 0.50980392 | 0.54384615 | 0.52173913 | 0.5 | 0.52173913 | 0.46153846 | 0.56 | colon.bma.model.cluster.moDC.RDS |
| 0.00236 | alt.negative.11:128443099, | | | | | | | | | | |
| 0 | ref.negative.11:128443099, | | | | | | | | | | |
| 0.00947 | ref.negative.14:98734567, | | | | | | | | | | |
| 0.00081 | alt.negative.9:117568766 | | | | | | | | | | |
| 0.00638 | ref.negative.14:98734567, | 3 | 0.90196078 | 0.52941176 | 0.48153846 | 0.52173913 | 0.6 | 0.52173913 | 0.92307692 | 0.12 | colon.bma.model.cluster.moDC.RDS |
| 0.00382 | alt.negative.9:117568766, | | | | | | | | | | |
| −0.00768 | ref.negative.21:45658474 | | | | | | | | | | |
| 0.00325 | ref.negative.20:62305274, | 3 | 0.90196078 | 0.52941176 | 0.46230769 | 0.52173913 | 0.6 | 0.52173913 | 0.92307692 | 0.12 | colon.bma.model.cluster.moDC.RDS |
| 0.00341 | alt.negative.9:117568766, | | | | | | | | | | |
| −0.00659 | ref.negative.21:45658474 | | | | | | | | | | |
| 0.01119 | haploinsuff.11:128443099, | 3 | 0.49019608 | 0.50980392 | 0.53846154 | 0.52 | 0.5 | 0.52 | 0.5 | 0.52 | colon.bma.model.cluster.moDC.RDS |
| 0.00297 | alt.negative.11:128443099, | | | | | | | | | | |
| 0 | ref.negative.11:128443099, | | | | | | | | | | |
| 0.00695 | ref.negative.5:11562221, | | | | | | | | | | |
| 0.00048 | ref.negative.20:62305274 | | | | | | | | | | |
| 0.00999 | haploinsuff.11:128443099, | 3 | 0.49019608 | 0.50980392 | 0.51384615 | 0.52 | 0.5 | 0.52 | 0.5 | 0.52 | colon.bma.model.cluster.moDC.RDS |
| 0.00244 | alt.negative.11:128443099, | | | | | | | | | | |
| 0 | ref.negative.11:128443099, | | | | | | | | | | |
| −1e−04 | ref.negative.20:62305274, | | | | | | | | | | |
| 0.00036 | alt.negative.9:117568766 | | | | | | | | | | |
| 0.01111 | haploinsuff.11:128443099, | 3 | 0.49019608 | 0.50980392 | 0.48615385 | 0.52 | 0.5 | 0.52 | 0.5 | 0.52 | colon.bma.model.cluster.moDC.RDS |
| 0.00215 | alt.negative.11:128443099, | | | | | | | | | | |
| 0 | ref.negative.11:128443099, | | | | | | | | | | |
| 0.00214 | alt.negative.9:117568766, | | | | | | | | | | |
| −0.00472 | ref.negative.21:45658474 | | | | | | | | | | |
| 0.01032 | ref.negative.5:11562221, | 3 | 0.96078431 | 0.50980392 | 0.44384615 | 0.51020408 | 0.5 | 0.51020408 | 0.96153846 | 0.04 | colon.bma.model.cluster.moDC.RDS |
| 0.00124 | alt.negative.9:117568766, | | | | | | | | | | |
| −0.00755 | ref.negative.21:45658474 | | | | | | | | | | |
| 0.00011 | additive.9:117568766, | 8 | 0.31372549 | 0.60784314 | 0.55615385 | 0.6875 | 0.57142857 | 0.6875 | 0.42307692 | 0.8 | colon.bma.model.cluster.Resi- |
| 0.00127 | alt.recessive.9:117568766, | | | | | | | | | | dent.macrophages.RDS |
| 0 | alt.negative.9:117568766, | | | | | | | | | | |
| 0.00156 | ref.recessive.21:45658474, | | | | | | | | | | |
| 0.00036 | ref.negative.21:45658474, | | | | | | | | | | |
| 0.00101 | ref.negative.5:11562221, | | | | | | | | | | |
| 0.00075 | ref.negative.20:62305274, | | | | | | | | | | |
| 0.00097 | ref.negative.11:128443099, | | | | | | | | | | |
| 0.00074 | ref.negative.14:98734567, | | | | | | | | | | |
| 0.00015 | alt.negative.16:11331509, | | | | | | | | | | |
| 0 | ref.negative.16:11331509, | | | | | | | | | | |
| 0.00106 | ref.negative.16:6147645 | | | | | | | | | | |
| 0.00018999087762517 | additive.9:117568766, | 8 | 0.33333333 | 0.54901961 | 0.53384615 | 0.58823529 | 0.52941176 | 0.58823529 | 0.38461538 | 0.72 | colon.bma.model.cluster.Resi- |
| 0.000333486194363657 | ref.recessive.21:45658474, | | | | | | | | | | dent.macrophages.RDS |
| 0.000227161557206912 | alt.recessive.9:117568766, | | | | | | | | | | |
| 9.98800079656857e−05 | alt.negative.9:117568766, | | | | | | | | | | |
| 0.000100275729953466 | ref.negative.21:45658474, | | | | | | | | | | |
| 8.64483570135836e−05 | ref.negative.5:11562221, | | | | | | | | | | |
| 8.02997304865149e−05 | ref.negative.20:62305274, | | | | | | | | | | |
| 7.93051794424818e−05 | ref.negative.11:128443099, | | | | | | | | | | |
| 6.36147622108668e−05 | ref.negative.14:98734567, | | | | | | | | | | |
| 3.98522874962439e−05 | alt.negative.16:11331509, | | | | | | | | | | |
| 3.2918936495849e−05 | ref.negative.16:6147645, | | | | | | | | | | |
| 2.45776244324862e−05 | ref.negative.16:11331509 | | | | | | | | | | |
| −3e−05 | additive.9:117568766, | 7 | 0.33333333 | 0.58823529 | 0.55461538 | 0.64705882 | 0.55882353 | 0.64705882 | 0.42307692 | 0.76 | colon.bma.model.cluster.Resi- |
| 0.00139 | alt.recessive.9:117568766, | | | | | | | | | | dent.macrophages.RDS |
| 0 | alt.negative.9:117568766, | | | | | | | | | | |
| 0.00154 | ref.recessive.21:45658474, | | | | | | | | | | |
| 0.00019 | ref.negative.21:45658474, | | | | | | | | | | |
| 0.00115 | ref.negative.5:11562221, | | | | | | | | | | |
| 0.00082 | ref.negative.20:62305274, | | | | | | | | | | |
| 0.00093 | ref.negative.11:128443099, | | | | | | | | | | |
| 0.00052 | ref.negative.14:98734567, | | | | | | | | | | |
| 0.00099 | ref.negative.16:6147645 | | | | | | | | | | |
| −4e−05 | additive.9:117568766, | 7 | 0.31372549 | 0.56862745 | 0.55076923 | 0.625 | 0.54285714 | 0.625 | 0.38461538 | 0.76 | colon.bma.model.cluster.Resi- |
| 0.00129 | alt.recessive.9:117568766, | | | | | | | | | | dent.macrophages.RDS |
| 0 | alt.negative.9:117568766, | | | | | | | | | | |
| 0.00145 | ref.recessive.21:45658474, | | | | | | | | | | |
| 0.00053 | ref.negative.21:45658474, | | | | | | | | | | |
| 0.00118 | ref.negative.5:11562221, | | | | | | | | | | |
| 0.00102 | ref.negative.11:128443099, | | | | | | | | | | |
| 0.00024 | ref.negative.14:98734567, | | | | | | | | | | |
| 0.00025 | alt.negative.16:11331509, | | | | | | | | | | |
| 4e−05 | ref.negative.16:11331509, | | | | | | | | | | |
| 0.001 | ref.negative.16:6147645 | | | | | | | | | | |
| −3e−05 | additive.9:117568766, | 7 | 0.31372549 | 0.52941176 | 0.53153846 | 0.5625 | 0.5428571 | 0.5625 | 0.34615385 | 0.72 | colon.bma.model.cluster.Resi- |
| 0.00114 | alt.recessive.9:117568766, | | | | | | | | | | dent.macrophages.RDS |
| 0 | alt.negative.9:117568766, | | | | | | | | | | |
| 0.00116 | ref.recessive.21:45658474, | | | | | | | | | | |
| 0.00037 | refnegative.21:45658474, | | | | | | | | | | |
| 0.00106 | ref.negative.5:11562221, | | | | | | | | | | |
| 0.00092 | ref.negative.20:62305274, | | | | | | | | | | |
| 0.00064 | ref.negative.11:128443099, | | | | | | | | | | |
| −0.00023 | ref.negative.14:98734567, | | | | | | | | | | |
| 0.00048 | alt.negative.16:11331509, | | | | | | | | | | |
| 0.00016 | ref.negative.16:11331509 | | | | | | | | | | |
| −2e−05 | additive.9:117568766, | 7 | 0.31372549 | 0.52941176 | 0.54153846 | 0.5625 | 0.51428571 | 0.5625 | 0.34615385 | 0.72 | colon.bma.model.cluster.Resi- |
| 0.00134 | alt.recessive.9:117568766, | | | | | | | | | | dent.macrophages.RDS |
| 0 | alt.negative.9:117568766, | | | | | | | | | | |
| 0.00141 | ref.recessive.21:45658474, | | | | | | | | | | |
| 0.00029 | refnegative.21:45658474, | | | | | | | | | | |
| 0.00097 | ref.negative.5:11562221, | | | | | | | | | | |
| 0.00075 | ref.negative.20:62305274, | | | | | | | | | | |
| 0.00097 | ref.negative.11:128443099, | | | | | | | | | | |
| 0.00035 | alt.negative.16:11331509, | | | | | | | | | | |
| 5e−05 | ref.negative.16:11331509, | | | | | | | | | | |
| 0.00101 | ref.negative.16:6147645 | | | | | | | | | | |
| 7e−05 | additive.9:117568766, | 7 | 0.3372549 | 0.52941176 | 0.51923077 | 0.5625 | 0.51428571 | 0.5625 | 0.34615385 | 0.72 | colon.bma.model.cluster.Resi- |
| 0.00118 | alt.recessive.9:117568766, | | | | | | | | | | dent.macrophages.RDS |
| 0 | alt.negative.9:117568766, | | | | | | | | | | |
| 0.00137 | ref.recessive.21:45658474, | | | | | | | | | | |
| 0.00016 | ref.negative.21:45658474, | | | | | | | | | | |
| 0.0011 | ref.negative.5:11562221, | | | | | | | | | | |
| 0.00129 | ref.negative.20:62305274, | | | | | | | | | | |
| 0.00059 | ref.negative.14:98734567, | | | | | | | | | | |
| 0.00034 | alt.negative.16:11331509, | | | | | | | | | | |
| −0.00027 | ref.negative.16:11331509, | | | | | | | | | | |
| 0.00098 | ref.negative.16:6147645 | | | | | | | | | | |
| 0.00024 | additive.9:117568766, | 7 | 0.31372549 | 0.52941176 | 0.54307692 | 0.5625 | 0.51428571 | 0.5625 | 0.34615385 | 0.72 | colon.bma.model.cluster.Resi- |
| 0.00108 | alt.recessive.9:117568766, | | | | | | | | | | dent.macrophages.RDS |
| 0 | alt.negative.9:117568766, | | | | | | | | | | |
| 0.00143 | ref.recessive.21:45658474, | | | | | | | | | | |
| 0.00057 | refnegative.21:45658474, | | | | | | | | | | |
| 0.00076 | ref.negative.20:62305274, | | | | | | | | | | |
| 0.00099 | ref.negative.11:128443099, | | | | | | | | | | |
| 0.00038 | refnegative.14:98734567, | | | | | | | | | | |
| 0.00018 | alt.negative.16:11331509, | | | | | | | | | | |
| −3e−05 | ref.negative.16:11331509, | | | | | | | | | | |
| 0.00099 | ref.negative.16:6147645 | | | | | | | | | | |
| 0.00158 | ref.recessive.21:45658474, | 7 | 0.31372549 | 0.52941176 | 0.52 | 0.5625 | 0.51428571 | 0.5625 | 0.34615385 | 0.72 | colon.bma.model.cluster.Resi- |
| 0.00085 | ref.negative.21:45658474, | | | | | | | | | | dent.macrophages.RDS |
| 0.00144 | ref.negative.5:11562221, | | | | | | | | | | |
| 0.00062 | ref.negative.20:62305274, | | | | | | | | | | |
| 0.00074 | ref.negative.11:128443099, | | | | | | | | | | |
| 0.00047 | ref.negative.14:98734567, | | | | | | | | | | |
| 5e−04 | alt.negative.16:11331509, | | | | | | | | | | |
| 0.00044 | ref.negative.16:11331509, | | | | | | | | | | |
| 0.00071 | ref.negative.16:6147645 | | | | | | | | | | |
| 0.00078 | additive.9:117568766, | 7 | 0.37254902 | 0.43137255 | 0.48923077 | 0.42105263 | 0.4375 | 0.42105263 | 0.30769231 | 0.56 | colon.bma.model.cluster.Resi- |
| 0.00074 | alt.recessive.9:117568766, | | | | | | | | | | dent.macrophages.RDS |
| 0 | alt.negative.9:117568766, | | | | | | | | | | |
| 0.00089 | ref.negative.5:11562221, | | | | | | | | | | |
| 0.00119 | ref.negative.20:62305274, | | | | | | | | | | |
| 0.00078 | ref.negative.11:128443099, | | | | | | | | | | |
| 0.00067 | ref.negative.14:98734567, | | | | | | | | | | |
| 0.00036 | alt.negative.16:11331509, | | | | | | | | | | |
| −2e−04 | ref.negative.16:11331509, | | | | | | | | | | |
| 0.00097 | ref.negative.16:6147645 | | | | | | | | | | |
| −1e−05 | additive.9:117568766, | 6 | 0.31372549 | 0.60784314 | 0.53307692 | 0.6875 | 0.57142857 | 0.6875 | 0.42307692 | 0.8 | colon.bma.model.cluster.Resi- |
| 0.00134 | alt.recessive.9:117568766, | | | | | | | | | | dent.macrophages.RDS |
| 0 | alt.negative.9:117568766, | | | | | | | | | | |
| 0.00152 | ref.recessive.21:45658474, | | | | | | | | | | |
| 0.00044 | ref.negative.21:45658474, | | | | | | | | | | |
| 0.00099 | ref.negative.5:11562221, | | | | | | | | | | |
| 0.00106 | ref.negative.11:128443099, | | | | | | | | | | |
| 0.00052 | ref.negative.14:98734567, | | | | | | | | | | |
| 0.0011 | ref.negative.16:6147645 | | | | | | | | | | |
| 3e−05 | additive.9:117568766, | 6 | 0.33333333 | 0.58823529 | 0.54461538 | 0.64705882 | 0.55882353 | 0.64705882 | 0.42307692 | 0.76 | colon.bma.model.cluster.Resi- |
| 0.00127 | alt.recessive.9:117568766, | | | | | | | | | | dent.macrophages.RDS |
| 0 | alt.negative.9:117568766, | | | | | | | | | | |
| 0.00145 | ref.recessive.21:45658474, | | | | | | | | | | |
| 0.00033 | refnegative.21:45658474, | | | | | | | | | | |
| 0.00087 | ref.negative.5:11562221, | | | | | | | | | | |
| 0.00067 | ref.negative.20:62305274, | | | | | | | | | | |
| 0.00106 | ref.negative.11:128443099, | | | | | | | | | | |
| 0.001 | ref.negative.16:6147645 | | | | | | | | | | |
| 0.00022 | additive.9:117568766, | 6 | 0.33333333 | 0.58823529 | 0.53076923 | 0.64705882 | 0.55882353 | 0.64705882 | 0.42307692 | 0.76 | colon.bma.model.cluster.Resi- |
| 0.00122 | alt.recessive.9:117568766, | | | | | | | | | | dent.macrophages.RDS |
| 0 | alt.negative.9:117568766, | | | | | | | | | | |
| 0.00153 | ref.recessive.21:45658474, | | | | | | | | | | |
| 0.00049 | refnegative.21:45658474, | | | | | | | | | | |
| 0.00109 | ref.negative.11:128443099, | | | | | | | | | | |
| 0.00074 | ref.negative.14:98734567, | | | | | | | | | | |
| 0.00034 | alt.negative.16:11331509, | | | | | | | | | | |
| 0.00015 | ref.negative.16:11331509, | | | | | | | | | | |
| 0.00119 | ref.negative.16:6147645 | | | | | | | | | | |
| 5e−05 | additive.9:117568766, | 6 | 0.31372549 | 0.56862745 | 0.53307692 | 0.625 | 0.54285714 | 0.625 | 0.38461538 | 0.76 | colon.bma.model.cluster.Resi- |
| 0.00115 | alt.recessive.9:117568766, | | | | | | | | | | dent.macrophages.RDS |
| 0 | alt.negative.9:117568766, | | | | | | | | | | |
| 0.00139 | ref.recessive.21:45658474, | | | | | | | | | | |
| 3e−04 | ref.negative.21:45658474, | | | | | | | | | | |
| 0.00125 | ref.negative.5:11562221, | | | | | | | | | | |
| 0.00086 | ref.negative.11:128443099, | | | | | | | | | | |
| 0 | ref.negative.14:98734567, | | | | | | | | | | |
| 0.00063 | alt.negative.16:11331509, | | | | | | | | | | |
| 0.00016 | ref.negative.16:11331509 | | | | | | | | | | |
| 1e−04 | additive.9:117568766, | 6 | 0.31372549 | 0.56862745 | 0.54461538 | 0.625 | 0.54285714 | 0.625 | 0.38461538 | 0.76 | colon.bma.model.cluster.Resi- |
| 0.00115 | alt.recessive.9:117568766, | | | | | | | | | | dent.macrophages.RDS |
| 0 | alt.negative.9:117568766, | | | | | | | | | | |
| 0.00152 | ref.recessive.21:45658474, | | | | | | | | | | |
| 0.00063 | ref.negative.21:45658474, | | | | | | | | | | |
| 0.00106 | ref.negative.5:11562221, | | | | | | | | | | |
| 0.00104 | ref.negative.11:128443099, | | | | | | | | | | |
| 0.00036 | alt.negative.16:11331509, | | | | | | | | | | |
| 0.00018 | ref.negative.16:11331509, | | | | | | | | | | |
| 0.00105 | ref.negative.16:6147645 | | | | | | | | | | |
| 0.00147 | ref.recessive.21:45658474, | 6 | 0.31372549 | 0.56862745 | 0.50230769 | 0.625 | 0.54285714 | 0.625 | 0.38461538 | 0.76 | colon.bma.model.cluster.Resi- |
| 0.00075 | ref.negative.21:45658474, | | | | | | | | | | dent.macrophages.RDS |
| 0.00132 | ref.negative.5:11562221, | | | | | | | | | | |
| 0.00047 | ref.negative.20:62305274, | | | | | | | | | | |
| 0.00082 | ref.negative.11:128443099, | | | | | | | | | | |
| −0.00026 | ref.negative.14:98734567, | | | | | | | | | | |
| 0.00061 | alt.negative.16:11331509, | | | | | | | | | | |
| 0.00045 | ref.negative.16:11331509 | | | | | | | | | | |
| −0.00023 | additive.9:117568766, | 6 | 0.35294118 | 0.56862745 | 0.56 | 0.61111111 | 0.54545455 | 0.61111111 | 0.42307692 | 0.72 | colon.bma.model.cluster.Resi- |
| 0.00154 | alt.recessive.9:117568766, | | | | | | | | | | dent.macrophages.RDS |
| 0 | alt.negative.9:117568766, | | | | | | | | | | |
| 0.00152 | ref.recessive.21:45658474, | | | | | | | | | | |
| 0.00031 | refnegative.21:45658474, | | | | | | | | | | |
| 0.00124 | ref.negative.5:11562221, | | | | | | | | | | |
| 0.00086 | ref.negative.20:62305274, | | | | | | | | | | |
| 0.00101 | ref.negative.11:128443099, | | | | | | | | | | |
| −5e−05 | ref.negative.14:98734567 | | | | | | | | | | |
| 0.00028 | additive.9:117568766, | 6 | 0.35294118 | 0.56862745 | 0.53923077 | 0.61111111 | 0.54545455 | 0.61111111 | 0.42307692 | 0.72 | colon.bma.model.cluster.Resi- |
| 0.00111 | alt.recessive.9:117568766, | | | | | | | | | | dent.macrophages.RDS |
| 0 | alt.negative.9:117568766, | | | | | | | | | | |
| 0.00157 | ref.recessive.21:45658474, | | | | | | | | | | |
| 5e−04 | ref.negative.21:45658474, | | | | | | | | | | |
| 0.00075 | ref.negative.20:62305274, | | | | | | | | | | |
| 0.00115 | ref.negative.11:128443099, | | | | | | | | | | |
| 0.00041 | refnegative.14:98734567, | | | | | | | | | | |
| 0.00115 | ref.negative.16:6147645 | | | | | | | | | | |
| 0.00157 | ref.recessive.21:45658474, | 6 | 0.39215686 | 0.56862745 | 0.52153846 | 0.6 | 0.5483871 | 0.6 | 0.46153846 | 0.68 | colon.bma.model.cluster.Resi- |
| 0.00072 | ref.negative.21:45658474, | | | | | | | | | | dent.macrophages.RDS |
| 0.00124 | ref.negative.5:11562221, | | | | | | | | | | |
| 5e−04 | ref.negative.20:62305274, | | | | | | | | | | |
| 0.00088 | ref.negative.11:128443099, | | | | | | | | | | |
| 2e−04 | ref.negative.14:98734567, | | | | | | | | | | |
| 0.00085 | ref.negative.16:6147645 | | | | | | | | | | |
| −1e−04 | additive.9:117568766, | 6 | 0.31372549 | 0.52941176 | 0.54076923 | 0.5625 | 0.51428571 | 0.5625 | 0.34615385 | 0.72 | colon.bma.model.cluster.Resi- |
| 0.00128 | alt.recessive.9:117568766, | | | | | | | | | | dent.macrophages.RDS |
| 0 | alt.negative.9:117568766, | | | | | | | | | | |
| 0.00143 | ref.recessive.21:45658474, | | | | | | | | | | |
| 0.00037 | ref.negative.21:45658474, | | | | | | | | | | |
| 0.00128 | ref.negative.5:11562221, | | | | | | | | | | |
| 0.00087 | ref.negative.20:62305274, | | | | | | | | | | |
| 0.00086 | ref.negative.11:128443099, | | | | | | | | | | |
| 0.00039 | alt.negative.16:11331509, | | | | | | | | | | |
| 0.00028 | ref.negative.16:11331509 | | | | | | | | | | |
| 0.00022 | additive.9:117568766, | 6 | 0.31372549 | 0.52941176 | 0.52769231 | 0.5625 | 0.51428571 | 0.5625 | 0.34615385 | 0.72 | colon.bma.model.cluster.Resi- |
| 0.00095 | alt.recessive.9:117568766, | | | | | | | | | | dent.macrophages.RDS |
| 0 | alt.negative.9:117568766, | | | | | | | | | | |
| 0.0013 | ref.recessive.21:45658474, | | | | | | | | | | |
| 0.00036 | ref.negative.21:45658474, | | | | | | | | | | |
| 0.00134 | ref.negative.5:11562221, | | | | | | | | | | |
| 0.00125 | ref.negative.20:62305274, | | | | | | | | | | |
| 0.00057 | alt.negative.16:11331509, | | | | | | | | | | |
| 0.00014 | ref.negative.16:11331509, | | | | | | | | | | |
| 0.00084 | ref.negative.16:6147645 | | | | | | | | | | |
| 0.00021 | additive.9:117568766, | 6 | 0.31372549 | 0.52941176 | 0.53076923 | 0.5625 | 0.51428571 | 0.5625 | 0.34615385 | 0.72 | colon.bma.model.cluster.Resi- |
| 0.00094 | alt.recessive.9:117568766, | | | | | | | | | | dent.macrophages.RDS |
| 0 | alt.negative.9:117568766, | | | | | | | | | | |
| 0.00128 | ref.recessive.21:45658474, | | | | | | | | | | |
| 0.00043 | ref.negative.21:45658474, | | | | | | | | | | |
| 0.00087 | ref.negative.20:62305274, | | | | | | | | | | |
| 0.00089 | ref.negative.11:128443099, | | | | | | | | | | |
| 0.00028 | ref.negative.14:98734567, | | | | | | | | | | |
| 0.00048 | alt.negative.16:11331509, | | | | | | | | | | |
| 0.00015 | ref.negative.16:11331509 | | | | | | | | | | |
| 0.00028 | additive.9:117568766, | 6 | 0.31372549 | 0.52941176 | 0.54230769 | 0.5625 | 0.51428571 | 0.5625 | 0.34615385 | 0.72 | colon.bma.model.cluster.Resi- |
| 0.00102 | alt.recessive.9:117568766, | | | | | | | | | | dent.macrophages.RDS |
| 0 | alt.negative.9:117568766, | | | | | | | | | | |
| 0.00137 | ref.recessive.21:45658474, | | | | | | | | | | |
| 0.00069 | ref.negative.21:45658474, | | | | | | | | | | |
| 0.00079 | ref.negative.20:62305274, | | | | | | | | | | |
| 0.00093 | ref.negative.11:128443099, | | | | | | | | | | |
| 4e−04 | alt.negative.16:11331509, | | | | | | | | | | |
| 6e−05 | ref.negative.16:11331509, | | | | | | | | | | |
| 0.00097 | ref.negative.16:6147645 | | | | | | | | | | |
| 0.00164 | ref.recessive.21:45658474, | 6 | 0.31372549 | 0.52941176 | 0.50846154 | 0.5625 | 0.51428571 | 0.5625 | 0.34615385 | 0.72 | colon.bma.model.cluster.Resi- |
| 0.00082 | ref.negative.21:45658474, | | | | | | | | | | dent.macrophages.RDS |
| 0.00134 | ref.negative.5:11562221, | | | | | | | | | | |
| 0.00034 | ref.negative.20:62305274, | | | | | | | | | | |
| 0.00095 | ref.negative.11:128443099, | | | | | | | | | | |
| 0.00051 | alt.negative.16:11331509, | | | | | | | | | | |
| 0.00035 | ref.negative.16:11331509, | | | | | | | | | | |
| 0.00088 | ref.negative.16:6147645 | | | | | | | | | | |
| 2e−05 | additive.9:117568766, | 6 | 0.35294118 | 0.52941176 | 0.53692308 | 0.55555556 | 0.51515152 | 0.55555556 | 0.38461538 | 0.68 | colon.bma.model.cluster.Resi- |
| 0.00115 | alt.recessive.9:117568766, | | | | | | | | | | dent.macrophages.RDS |
| 0 | alt.negative.9:117568766, | | | | | | | | | | |
| 0.00132 | ref.recessive.21:45658474, | | | | | | | | | | |
| 0.00028 | ref.negative.21:45658474, | | | | | | | | | | |
| 0.00142 | ref.negative.5:11562221, | | | | | | | | | | |
| 0.00163 | ref.negative.20:62305274, | | | | | | | | | | |
| 0.00024 | ref.negative.14:98734567, | | | | | | | | | | |
| 0.00065 | alt.negative.16:11331509, | | | | | | | | | | |
| 0.00014 | ref.negative.16:11331509 | | | | | | | | | | |
| −4e−05 | additive.9:117568766, | 6 | 0.33333333 | 0.50980392 | 0.53615385 | 0.52941176 | 0.5 | 0.52941176 | 0.34615385 | 0.68 | colon.bma.model.cluster.Resi- |
| 0.00129 | alt.recessive.9:117568766, | | | | | | | | | | dent.macrophages.RDS |
| 0 | alt.negative.9:117568766, | | | | | | | | | | |
| 0.00142 | ref.recessive.21:45658474, | | | | | | | | | | |
| 0.00013 | refnegative.21:45658474, | | | | | | | | | | |
| 0.00122 | ref.negative.5:11562221, | | | | | | | | | | |
| 0.00139 | ref.negative.20:62305274, | | | | | | | | | | |
| 4e−04 | ref.negative.14:98734567, | | | | | | | | | | |
| 0.00096 | ref.negative.16:6147645 | | | | | | | | | | |
| 0.00027 | additive.9:117568766, | 6 | 0.33333333 | 0.50980392 | 0.52 | 0.52941176 | 0.5 | 0.52941176 | 0.34615385 | 0.68 | colon.bma.model.cluster.Resi- |
| 0.00097 | alt.recessive.9:117568766, | | | | | | | | | | dent.macrophages.RDS |
| 0 | alt.negative.9:117568766, | | | | | | | | | | |
| 0.0012 | ref.recessive.21:45658474, | | | | | | | | | | |
| 0.00041 | refnegative.21:45658474, | | | | | | | | | | |
| 0.00135 | ref.negative.20:62305274, | | | | | | | | | | |
| 0.00061 | ref.negative.14:98734567, | | | | | | | | | | |
| 0.00016 | alt.negative.16:11331509, | | | | | | | | | | |
| −0.00023 | ref.negative.16:11331509, | | | | | | | | | | |
| 0.00088 | ref.negative.16:6147645 | | | | | | | | | | |
| 0.00147 | ref.negative.5:11562221, | 6 | 0.37254902 | 0.50980392 | 0.52384615 | 0.52631579 | 0.5 | 0.52631579 | 0.38461538 | 0.64 | colon.bma.model.cluster.Resi- |
| 0.00065 | ref.negative.20:62305274, | | | | | | | | | | dent.macrophages.RDS |
| 0.00055 | ref.negative.11:128443099, | | | | | | | | | | |
| 0.00047 | ref.negative.14:98734567, | | | | | | | | | | |
| 0.00052 | alt.negative.16:11331509, | | | | | | | | | | |
| 4e−04 | ref.negative.16:11331509, | | | | | | | | | | |
| 0.00059 | ref.negative.16:6147645 | | | | | | | | | | |
| 1e−05 | additive.9:117568766, | 6 | 0.31372549 | 0.49019608 | 0.50307692 | 0.5 | 0.48571429 | 0.5 | 0.30769231 | 0.68 | colon.bma.model.cluster.Resi- |
| 0.00137 | alt.recessive.9:117568766, | | | | | | | | | | dent.macrophages.RDS |
| 0 | alt.negative.9:117568766, | | | | | | | | | | |
| 0.00143 | ref.recessive.21:45658474, | | | | | | | | | | |
| 4e−05 | ref.negative.21:45658474, | | | | | | | | | | |
| 0.00147 | ref.negative.5:11562221, | | | | | | | | | | |
| 0.00074 | ref.negative.14:98734567, | | | | | | | | | | |
| 3e−04 | alt.negative.16:11331509, | | | | | | | | | | |
| 6e−05 | ref.negative.16:11331509, | | | | | | | | | | |
| 0.00096 | ref.negative.16:6147645 | | | | | | | | | | |
| 0.00163 | ref.recessive.21:45658474, | 6 | 0.35294118 | 0.49019608 | 0.49 | 0.5 | 0.48484848 | 0.5 | 0.34615385 | 0.64 | colon.bma.model.cluster.Resi- |
| 0.00065 | ref.negative.21:45658474, | | | | | | | | | | dent.macrophages.RDS |
| 0.00162 | ref.negative.5:11562221, | | | | | | | | | | |
| 0.00089 | ref.negative.20:62305274, | | | | | | | | | | |
| 0.00058 | ref.negative.14:98734567, | | | | | | | | | | |
| 0.00045 | alt.negative.16:11331509, | | | | | | | | | | |
| 0.00043 | ref.negative.16:11331509, | | | | | | | | | | |
| 0.00093 | ref.negative.16:6147645 | | | | | | | | | | |
| 0.00153 | ref.recessive.21:45658474, | 6 | 0.31372549 | 0.49019608 | 0.50461538 | 0.5 | 0.48571429 | 0.5 | 0.30769231 | 0.68 | colon.bma.model.cluster.Resi- |
| 9e−04 | ref.negative.21:45658474, | | | | | | | | | | dent.macrophages.RDS |
| 0.00129 | ref.negative.5:11562221, | | | | | | | | | | |
| 0.00092 | ref.negative.11:128443099, | | | | | | | | | | |
| 0.00026 | ref.negative.14:98734567, | | | | | | | | | | |
| 0.00051 | alt.negative.16:11331509, | | | | | | | | | | |
| 0.00024 | ref.negative.16:11331509, | | | | | | | | | | |
| 0.00082 | ref.negative.16:6147645 | | | | | | | | | | |
| 0.00146 | ref.recessive.21:45658474, | 6 | 0.31372549 | 0.49019608 | 0.5769231 | 0.5 | 0.48571429 | 0.5 | 0.30769231 | 0.68 | colon.bma.model.cluster.Resi- |
| 0.00115 | ref.negative.21:45658474, | | | | | | | | | | dent.macrophages.RDS |
| 0.00052 | ref.negative.20:62305274, | | | | | | | | | | |
| 0.00085 | ref.negative.11:128443099, | | | | | | | | | | |
| 0.00043 | ref.negative.14:98734567, | | | | | | | | | | |
| 0.00035 | alt.negative.16:11331509, | | | | | | | | | | |
| 0.00051 | ref.negative.16:11331509, | | | | | | | | | | |
| 9e−04 | ref.negative.16:6147645 | | | | | | | | | | |
| 0.00065 | additive.9:117568766, | 6 | 0.45098039 | 0.47058824 | 0.50692308 | 0.47826087 | 0.46428571 | 0.47826087 | 0.42307692 | 0.52 | colon.bma.model.cluster.Resi- |
| 0.00076 | alt.recessive.9:117568766, | | | | | | | | | | dent.macrophages.RDS |
| 0 | alt.negative.9:117568766, | | | | | | | | | | |
| 8e−04 | ref.negative.5:11562221, | | | | | | | | | | |
| 0.00108 | ref.negative.20:62305274, | | | | | | | | | | |
| 0.00078 | ref.negative.11:128443099, | | | | | | | | | | |
| 0.00075 | ref.negative.14:98734567, | | | | | | | | | | |
| 0.00085 | ref.negative.16:6147645 | | | | | | | | | | |
| 0.00065 | additive.9:117568766, | 6 | 0.43137255 | 0.45098039 | 0.50461538 | 0.45454545 | 0.44827586 | 0.45454545 | 0.38461538 | 0.52 | colon.bma.model.cluster.Resi- |
| 0.00054 | alt.recessive.9:117568766, | | | | | | | | | | dent.macrophages.RDS |
| 0 | alt.negative.9:117568766, | | | | | | | | | | |
| 0.00086 | ref.negative.5:11562221, | | | | | | | | | | |
| 0.00102 | ref.negative.20:62305274, | | | | | | | | | | |
| 0.00062 | ref.negative.11:128443099, | | | | | | | | | | |
| 0.00029 | ref.negative.14:98734567, | | | | | | | | | | |
| 0.00055 | alt.negative.16:11331509, | | | | | | | | | | |
| −7e−05 | ref.negative.16:11331509 | | | | | | | | | | |
| 0.00079 | additive.9:117568766, | 6 | 0.31372549 | 0.45098039 | 0.47846154 | 0.4375 | 0.45714286 | 0.4375 | 0.26923077 | 0.64 | colon.bma.model.cluster.Resi- |
| 0.00055 | alt.recessive.9:117568766, | | | | | | | | | | dent.macrophages.RDS |
| 0 | alt.negative.9:117568766, | | | | | | | | | | |
| 0.00082 | ref.negative.5:11562221, | | | | | | | | | | |
| 0.001 | ref.negative.20:62305274, | | | | | | | | | | |
| 0.00072 | ref.negative.11:128443099, | | | | | | | | | | |
| 0.00055 | alt.negative.16:11331509, | | | | | | | | | | |
| −0.00027 | ref.negative.16:11331509, | | | | | | | | | | |
| 8e−04 | ref.negative.16:6147645 | | | | | | | | | | |
| 0.00081 | additive.9:117568766, | 6 | 0.31372549 | 0.45098039 | 0.48307692 | 0.4375 | 0.45714286 | 0.4375 | 0.26923077 | 0.64 | colon.bma.model.cluster.Resi- |
| 0.00065 | alt.recessive.9:117568766, | | | | | | | | | | dent.macrophages.RDS |
| 0 | alt.negative.9:117568766, | | | | | | | | | | |
| 0.00089 | ref.negative.20:62305274, | | | | | | | | | | |
| 0.00078 | ref.negative.11:128443099, | | | | | | | | | | |
| 0.00042 | refnegative.14:98734567, | | | | | | | | | | |
| 0.00051 | alt.negative.16:11331509, | | | | | | | | | | |
| −2e−04 | ref.negative.16:11331509, | | | | | | | | | | |
| 9e−04 | ref.negative.16:6147645 | | | | | | | | | | |
| 0.00085 | additive.9:117568766, | 6 | 0.37254902 | 0.43137255 | 0.48923077 | 0.42105263 | 0.4375 | 0.42105263 | 0.30769231 | 0.56 | colon.bma.model.cluster.Resi- |
| 0.00061 | alt.recessive.9:117568766, | | | | | | | | | | dent.macrophages.RDS |
| 0 | alt.negative.9:117568766, | | | | | | | | | | |
| 0.00101 | ref.negative.5:11562221, | | | | | | | | | | |
| 0.00177 | ref.negative.20:62305274, | | | | | | | | | | |
| 0.00073 | ref.negative.14:98734567, | | | | | | | | | | |
| 0.00062 | alt.negative.16:11331509, | | | | | | | | | | |
| −5e−05 | ref.negative.16:11331509, | | | | | | | | | | |
| 0.00098 | ref.negative.16:6147645 | | | | | | | | | | |
| 6e−04 | additive.9:117568766, | 6 | 0.35294118 | 0.41176471 | 0.49307692 | 0.38888889 | 0.42424242 | 0.38888889 | 0.26923077 | 0.56 | colon.bma.model.cluster.Resi- |
| 0.00087 | alt.recessive.9:117568766, | | | | | | | | | | dent.macrophages.RDS |
| 0 | alt.negative.9:117568766, | | | | | | | | | | |
| 9e−04 | ref.negative.5:11562221, | | | | | | | | | | |
| 0.00088 | ref.negative.11:128443099, | | | | | | | | | | |
| 0.00035 | ref.negative.14:98734567, | | | | | | | | | | |
| 3e−04 | alt.negative.16:11331509, | | | | | | | | | | |
| −0.00013 | ref.negative.16:11331509, | | | | | | | | | | |
| 0.00068 | ref.negative.16:6147645 | | | | | | | | | | |
| −4e−05 | additive.9:117568766, | 5 | 0.35294118 | 0.60784314 | 0.51538462 | 0.66666667 | 0.57575758 | 0.66666667 | 0.46153846 | 0.76 | colon.bma.model.cluster.Resi- |
| 0.00135 | alt.recessive.9:117568766, | | | | | | | | | | dent.macrophages.RDS |
| 0 | alt.negative.9:117568766, | | | | | | | | | | |
| 0.00146 | ref.recessive.21:45658474, | | | | | | | | | | |
| 0.00036 | refnegative.21:45658474, | | | | | | | | | | |
| 0.00102 | ref.negative.5:11562221, | | | | | | | | | | |
| 0.00111 | ref.negative.11:128443099, | | | | | | | | | | |
| 0.00112 | ref.negative.16:6147645 | | | | | | | | | | |
| 5e−05 | additive.9:117568766, | 5 | 0.33333333 | 0.58823529 | 0.53153846 | 0.64705882 | 0.55882353 | 0.64705882 | 0.42307692 | 0.76 | colon.bma.model.cluster.Resi- |
| 0.00139 | alt.recessive.9:117568766, | | | | | | | | | | dent.macrophages.RDS |
| 0 | alt.negative.9:117568766, | | | | | | | | | | |
| 0.00154 | ref.recessive.21:45658474, | | | | | | | | | | |
| 0.00036 | ref.negative.21:45658474, | | | | | | | | | | |
| 0.00119 | ref.negative.11:128443099, | | | | | | | | | | |
| 0.00053 | ref.negative.14:98734567, | | | | | | | | | | |
| 0.0011 | ref.negative.16:6147645 | | | | | | | | | | |
| 0.00158 | ref.recessive.21:45658474, | 5 | 0.31372549 | 0.56862745 | 0.52384615 | 0.625 | 0.54285714 | 0.625 | 0.38461538 | 0.76 | colon.bma.model.cluster.Resi- |
| 0.00065 | ref.negative.21:45658474, | | | | | | | | | | dent.macrophages.RDS |
| 0.0013 | ref.negative.5:11562221, | | | | | | | | | | |
| 0.00061 | ref.negative.20:62305274, | | | | | | | | | | |
| 0.00096 | ref.negative.11:128443099, | | | | | | | | | | |
| 0.00014 | ref.negative.14:98734567 | | | | | | | | | | |
| 0.00155 | ref.recessive.21:45658474, | 5 | 0.31372549 | 0.56862745 | 0.51846154 | 0.625 | 0.54285714 | 0.625 | 0.38461538 | 0.76 | colon.bma.model.cluster.Resi- |
| 8e−04 | ref.negative.21:45658474, | | | | | | | | | | dent.macrophages.RDS |
| 0.00146 | ref.negative.5:11562221, | | | | | | | | | | |
| 0.00059 | ref.negative.20:62305274, | | | | | | | | | | |
| 8e−04 | ref.negative.11:128443099, | | | | | | | | | | |
| 0.00066 | alt.negative.16:11331509, | | | | | | | | | | |
| 0.00064 | ref.negative.16:11331509 | | | | | | | | | | |
| 0.00175 | ref.recessive.21:45658474, | 5 | 0.31372549 | 0.56862745 | 0.52076923 | 0.625 | 0.54285714 | 0.625 | 0.38461538 | 0.76 | colon.bma.model.cluster.Resi- |
| 7e−04 | ref.negative.21:45658474, | | | | | | | | | | dent.macrophages.RDS |
| 0.0013 | ref.negative.5:11562221, | | | | | | | | | | |
| 0.00032 | ref.negative.20:62305274, | | | | | | | | | | |
| 0.00108 | ref.negative.11:128443099, | | | | | | | | | | |
| 0.00103 | ref.negative.16:6147645 | | | | | | | | | | |
| 0.00157 | ref.recessive.21:45658474, | 5 | 0.31372549 | 0.56862745 | 0.51384615 | 0.625 | 0.54285714 | 0.625 | 0.38461538 | 0.76 | colon.bma.model.cluster.Resi- |
| 0.00067 | ref.negative.21:45658474, | | | | | | | | | | dent.macrophages.RDS |
| 0.00129 | ref.negative.5:11562221, | | | | | | | | | | |
| 0.00087 | ref.negative.11:128443099, | | | | | | | | | | |
| 5e−05 | ref.negative.14:98734567, | | | | | | | | | | |
| 6e−04 | alt.negative.16:11331509, | | | | | | | | | | |
| 0.00052 | ref.negative.16:11331509 | | | | | | | | | | |
| −0.00025 | additive.9:117568766, | 5 | 0.35294118 | 0.56862745 | 0.55307692 | 0.61111111 | 0.54545455 | 0.61111111 | 0.42307692 | 0.72 | colon.bma.model.cluster.Resi- |
| 0.00169 | alt.recessive.9:117568766, | | | | | | | | | | dent.macrophages.RDS |
| 0 | alt.negative.9:117568766, | | | | | | | | | | |
| 0.00149 | ref.recessive.21:45658474, | | | | | | | | | | |
| 8e−05 | ref.negative.21:45658474, | | | | | | | | | | |
| 0.001 | ref.negative.5:11562221, | | | | | | | | | | |
| 0.00092 | ref.negative.20:62305274, | | | | | | | | | | |
| 0.00102 | ref.negative.11:128443099 | | | | | | | | | | |
| −0.00017 | additive.9:117568766, | 5 | 0.35294118 | 0.56862745 | 0.54384615 | 0.61111111 | 0.54545455 | 0.61111111 | 0.42307692 | 0.72 | colon.bma.model.cluster.Resi- |
| 0.00141 | alt.recessive.9:117568766, | | | | | | | | | | dent.macrophages.RDS |
| 0 | alt.negative.9:117568766, | | | | | | | | | | |
| 0.00143 | ref.recessive.21:45658474, | | | | | | | | | | |
| 0.00026 | ref.negative.21:45658474, | | | | | | | | | | |
| 0.00103 | ref.negative.5:11562221, | | | | | | | | | | |
| 0.00104 | ref.negative.11:128443099, | | | | | | | | | | |
| −2e−05 | ref.negative.14:98734567 | | | | | | | | | | |
| 0.00019 | additive.9:117568766, | 5 | 0.35294118 | 0.56862745 | 0.53846154 | 0.61111111 | 0.54545455 | 0.61111111 | 0.42307692 | 0.72 | colon.bma.model.cluster.Resi- |
| 0.0011 | alt.recessive.9:117568766, | | | | | | | | | | dent.macrophages.RDS |
| 0 | alt.negative.9:117568766, | | | | | | | | | | |
| 0.00156 | ref.recessive.21:45658474, | | | | | | | | | | |
| 0.00052 | refnegative.21:45658474, | | | | | | | | | | |
| 0.00073 | ref.negative.20:62305274, | | | | | | | | | | |
| 0.00116 | ref.negative.11:128443099, | | | | | | | | | | |
| 0.00107 | ref.negative.16:6147645 | | | | | | | | | | |
| 0.00134 | ref.recessive.21:45658474, | 5 | 0.43137255 | 0.56862745 | 0.53153846 | 0.59090909 | 0.55172414 | 0.59090909 | 0.5 | 0.64 | colon.bma.model.cluster.Resi- |
| 0.00064 | ref.negative.21:45658474, | | | | | | | | | | dent.macrophages.RDS |
| 0.00163 | ref.negative.5:11562221, | | | | | | | | | | |
| 0.00119 | ref.negative.20:62305274, | | | | | | | | | | |
| 0.00022 | ref.negative.14:98734567, | | | | | | | | | | |
| 7e−04 | alt.negative.16:11331509, | | | | | | | | | | |
| 0.00043 | ref.negative.16:11331509 | | | | | | | | | | |
| −8e−05 | additive.9:117568766, | 5 | 0.33333333 | 0.54901961 | 0.52538462 | 0.58823529 | 0.52941176 | 0.58823529 | 0.38461538 | 0.72 | colon.bma.model.cluster.Resi- |
| 0.00127 | alt.recessive.9:117568766, | | | | | | | | | | dent.macrophages.RDS |
| 0 | alt.negative.9:117568766, | | | | | | | | | | |
| 0.00146 | ref.recessive.21:45658474, | | | | | | | | | | |
| 0.00024 | refnegative.21:45658474, | | | | | | | | | | |
| 0.00109 | ref.negative.5:11562221, | | | | | | | | | | |
| 9e−04 | ref.negative.11:128443099, | | | | | | | | | | |
| 0.00059 | alt.negative.16:11331509, | | | | | | | | | | |
| 0.00041 | ref.negative.16:11331509 | | | | | | | | | | |
| 0.0015 | ref.recessive.21:45658474, | 5 | 0.33333333 | 0.54901961 | 0.52461538 | 0.58823529 | 0.52941176 | 0.58823529 | 0.38461538 | 0.72 | colon.bma.model.cluster.Resi- |
| 0.00092 | ref.negative.21:45658474, | | | | | | | | | | dent.macrophages.RDS |
| 0.00052 | ref.negative.20:62305274, | | | | | | | | | | |
| 0.00081 | ref.negative.11:128443099, | | | | | | | | | | |
| 0.00023 | ref.negative.14:98734567, | | | | | | | | | | |
| 0.00039 | alt.negative.16:11331509, | | | | | | | | | | |
| 0.00044 | ref.negative.16:11331509 | | | | | | | | | | |
| 3e−05 | additive.9:117568766, | 5 | 0.37254902 | 0.54901961 | 0.53923077 | 0.57894737 | 0.53125 | 0.57894737 | 0.42307692 | 0.68 | colon.bma.model.cluster.Resi- |
| 0.00118 | alt.recessive.9:117568766, | | | | | | | | | | dent.macrophages.RDS |
| 0 | alt.negative.9:117568766, | | | | | | | | | | |
| 0.0013 | ref.recessive.21:45658474, | | | | | | | | | | |
| 0.00045 | refnegative.21:45658474, | | | | | | | | | | |
| 0.00072 | ref.negative.20:62305274, | | | | | | | | | | |
| 0.00091 | ref.negative.11:128443099, | | | | | | | | | | |
| 1e−05 | ref.negative.14:98734567 | | | | | | | | | | |
| −0.00016 | additive.9:117568766, | 5 | 0.45098039 | 0.54901961 | 0.53 | 0.56521739 | 0,53571429 | 0.56521739 | 0.5 | 0.6 | colon.bma.model.cluster.Resi- |
| 0.00154 | alt.recessive.9:117568766, | | | | | | | | | | dent.macrophages.RDS |
| 0 | alt.negative.9:117568766, | | | | | | | | | | |
| 0.00149 | ref.recessive.21:45658474, | | | | | | | | | | |
| −1e−05 | ref.negative.21:45658474, | | | | | | | | | | |
| 0.00128 | ref.negative.5:11562221, | | | | | | | | | | |
| 0.00153 | ref.negative.20:62305274, | | | | | | | | | | |
| −1e−05 | ref.negative.14:98734567 | | | | | | | | | | |
| 0.00028 | additive.9:117568766, | 5 | 0.31372549 | 0.52941176 | 0.53692308 | 0.5625 | 0.51428571 | 0.5625 | 0.34615385 | 0.72 | colon.bma.model.cluster.Resi- |
| 0.00086 | alt.recessive.9:117568766, | | | | | | | | | | dent.macrophages.RDS |
| 0 | alt.negative.9:117568766, | | | | | | | | | | |
| 0.00116 | ref.recessive.21:45658474, | | | | | | | | | | |
| 0.00051 | ref.negative.21:45658474, | | | | | | | | | | |
| 0.00089 | ref.negative.20:62305274, | | | | | | | | | | |
| 0.00082 | ref.negative.11:128443099, | | | | | | | | | | |
| 0.00034 | alt.negative.16:11331509, | | | | | | | | | | |
| 1e−04 | ref.negative.16:11331509 | | | | | | | | | | |
| 0.00018 | additive.9:117568766, | 5 | 0.31372549 | 0.52941176 | 0.52307692 | 0.5625 | 0.51428571 | 0.5625 | 0.34615385 | 0.72 | colon.bma.model.cluster.Resi- |
| 0.00088 | alt.recessive.9:117568766, | | | | | | | | | | dent.macrophages.RDS |
| 0 | alt.negative.9:117568766, | | | | | | | | | | |
| 0.00128 | ref.recessive.21:45658474, | | | | | | | | | | |
| 0.00047 | ref.negative.21:45658474, | | | | | | | | | | |
| 0.00096 | ref.negative.11:128443099, | | | | | | | | | | |
| 3e−05 | ref.negative.14:98734567, | | | | | | | | | | |
| 0.00044 | alt.negative.16:11331509, | | | | | | | | | | |
| 0.00011 | ref.negative.16:11331509 | | | | | | | | | | |
| 0.00018 | additive.9:117568766, | 5 | 0.31372549 | 0.52941176 | 0.52230769 | 0.5625 | 0.51428571 | 0.5625 | 0.34615385 | 0.72 | colon.bma.model.cluster.Resi- |
| 0.00124 | alt.recessive.9:117568766, | | | | | | | | | | dent.macrophages.RDS |
| 0 | alt.negative.9:117568766, | | | | | | | | | | |
| 0.00151 | ref.recessive.21:45658474, | | | | | | | | | | |
| 0.00037 | ref.negative.21:45658474, | | | | | | | | | | |
| 0.00113 | ref.negative.11:128443099, | | | | | | | | | | |
| 0.00014 | alt.negative.16:11331509, | | | | | | | | | | |
| −0.00015 | ref.negative.16:11331509, | | | | | | | | | | |
| 0.00103 | ref.negative.16:6147645 | | | | | | | | | | |
| 0.00151 | ref.recessive.21:45658474, | 5 | 0.31372549 | 0.52941176 | 0.50846154 | 0.5625 | 0.51428571 | 0.5625 | 0.34615385 | 0.72 | colon.bma.model.cluster.Resi- |
| 0.00094 | ref.negative.21:45658474, | | | | | | | | | | dent.macrophages.RDS |
| 0.00038 | ref.negative.20:62305274, | | | | | | | | | | |
| 0.00098 | ref.negative.11:128443099, | | | | | | | | | | |
| 0.00031 | ref.negative.14:98734567, | | | | | | | | | | |
| 0.00094 | ref.negative.16:6147645 | | | | | | | | | | |
| −0.00017 | additive.9:117568766, | 5 | 0.35294118 | 0.52941176 | 0.53923077 | 0.55555556 | 0.51515152 | 0.55555556 | 0.38461538 | 0.68 | colon.bma.model.cluster.Resi- |
| 0.00135 | alt.recessive.9:117568766, | | | | | | | | | | dent.macrophages.RDS |
| 0 | alt.negative.9:117568766, | | | | | | | | | | |
| 0.00149 | ref.recessive.21:45658474, | | | | | | | | | | |
| 0.00031 | refnegative.21:45658474, | | | | | | | | | | |
| 0.00158 | ref.negative.5:11562221, | | | | | | | | | | |
| 0.00149 | ref.negative.20:62305274, | | | | | | | | | | |
| 0.00057 | alt.negative.16:11331509, | | | | | | | | | | |
| 0.00033 | ref.negative.16:11331509 | | | | | | | | | | |
| 4e−05 | additive.9:117568766, | 5 | 0.35294118 | 0.52941176 | 0.53153846 | 0.55555556 | 0.51515152 | 0.55555556 | 0.38461538 | 0.68 | colon.bma.model.cluster.Resi- |
| 0.00126 | alt.recessive.9:117568766, | | | | | | | | | | dent.macrophages.RDS |
| 0 | alt.negative.9:117568766, | | | | | | | | | | |
| 0.00147 | ref.recessive.21:45658474, | | | | | | | | | | |
| 8e−05 | ref.negative.21:45658474, | | | | | | | | | | |
| 0.00119 | ref.negative.5:11562221, | | | | | | | | | | |
| 0.00132 | ref.negative.20:62305274, | | | | | | | | | | |
| 0.00093 | ref.negative.16:6147645 | | | | | | | | | | |
| 5e−05 | additive.9:117568766, | 5 | 0.43137255 | 0.52941176 | 0.52615385 | 0.54545455 | 0.51724138 | 0.54545455 | 0.46153846 | 0.6 | colon.bma.model.cluster.Resi- |
| 0.00126 | alt.recessive.9:117568766, | | | | | | | | | | dent.macrophages.RDS |
| 0 | alt.negative.9:117568766, | | | | | | | | | | |
| 0.00125 | ref.recessive.21:45658474, | | | | | | | | | | |
| 0.00024 | ref.negative.21:45658474, | | | | | | | | | | |
| 0.00139 | ref.negative.20:62305274, | | | | | | | | | | |
| 0.00028 | ref.negative.14:98734567, | | | | | | | | | | |
| 0.00041 | alt.negative.16:11331509, | | | | | | | | | | |
| 0.00011 | ref.negative.16:11331509 | | | | | | | | | | |
| 0.00164 | ref.recessive.21:45658474, | 5 | 0.43137255 | 0.52941176 | 0.50230769 | 0.54545455 | 0.51724138 | 0.54545455 | 0.46153846 | 0.6 | colon.bma.model.cluster.Resi- |
| 0.00051 | ref.negative.21:45658474, | | | | | | | | | | dent.macrophages.RDS |
| 0.00131 | ref.negative.5:11562221, | | | | | | | | | | |
| 0.00101 | ref.negative.20:62305274, | | | | | | | | | | |
| 0.00048 | ref.negative.14:98734567, | | | | | | | | | | |
| 9e−04 | ref.negative.16:6147645 | | | | | | | | | | |
| 0.00157 | ref.recessive.21:45658474, | 5 | 0.43137255 | 0.52941176 | 0.51769231 | 0.54545455 | 0.51724138 | 0.54545455 | 0.46153846 | 0.6 | colon.bma.model.cluster.Resi- |
| 0.00063 | ref.negative.21:45658474, | | | | | | | | | | dent.macrophages.RDS |
| 0.0012 | ref.negative.5:11562221, | | | | | | | | | | |
| 0.00094 | ref.negative.11:128443099, | | | | | | | | | | |
| 0.00025 | ref.negative.14:98734567, | | | | | | | | | | |
| 0.00088 | ref.negative.16:6147645 | | | | | | | | | | |
| 0.0015 | ref.recessive.21:45658474, | 5 | 0.33333333 | 0.50980392 | 0.51461538 | 0.52941176 | 0.5 | 0.52941176 | 0.34615385 | 0.68 | colon.bma.model.cluster.Resi- |
| 0.00098 | ref.negative.21:45658474, | | | | | | | | | | dent.macrophages.RDS |
| 0.00113 | ref.negative.5:11562221, | | | | | | | | | | |
| 0.00093 | ref.negative.11:128443099, | | | | | | | | | | |
| 0.00058 | alt.negative.16:11331509, | | | | | | | | | | |
| 0.00067 | ref.negative.16:11331509, | | | | | | | | | | |
| 0.00087 | ref.negative.16:6147645 | | | | | | | | | | |
| 0.00157 | ref.recessive.21:45658474, | 5 | 0.33333333 | 0.50980392 | 0.51153846 | 0.52941176 | 0.5 | 0.52941176 | 0.34615385 | 0.68 | colon.bma.model.cluster.Resi- |
| 0.00104 | ref.negative.21:45658474, | | | | | | | | | | dent.macrophages.RDS |
| 0.00044 | ref.negative.20:62305274, | | | | | | | | | | |
| 0.00099 | ref.negative.11:128443099, | | | | | | | | | | |
| 3e−04 | alt.negative.16:11331509, | | | | | | | | | | |
| 0.00032 | ref.negative.16:11331509, | | | | | | | | | | |
| 0.00075 | ref.negative.16:6147645 | | | | | | | | | | |
| 0.0014 | ref.recessive.21:45658474, | 5 | 0.37254902 | 0.50980392 | 0.49153846 | 0.52631579 | 0.5 | 0.52631579 | 0.38461538 | 0.64 | colon.bma.model.cluster.Resi- |
| 0.00053 | ref.negative.21:45658474, | | | | | | | | | | dent.macrophages.RDS |
| 0.00165 | ref.negative.5:11562221, | | | | | | | | | | |
| 0.00093 | ref.negative.20:62305274, | | | | | | | | | | |
| 0.00068 | alt.negative.16:11331509, | | | | | | | | | | |
| 0.00028 | ref.negative.16:11331509, | | | | | | | | | | |
| 0.00068 | ref.negative.16:6147645 | | | | | | | | | | |
| 0.0016 | ref.negative.5:11562221, | 5 | 0.41176471 | 0.50980392 | 0.53 | 0.52380952 | 0.5 | 0.52380952 | 0.42307692 | 0.6 | colon.bma.model.cluster.Resi- |
| 0.00115 | ref.negative.20:62305274, | | | | | | | | | | dent.macrophages.RDS |
| 0.00072 | ref.negative.14:98734567, | | | | | | | | | | |
| 6e−04 | alt.negative.16:11331509, | | | | | | | | | | |
| 0.00021 | ref.negative.16:11331509, | | | | | | | | | | |
| 0.00072 | ref.negative.16:6147645 | | | | | | | | | | |
| −0.00012 | additive.9:117568766, | 5 | 0.39215686 | 0.49019608 | 0.49307692 | 0.5 | 0.48387097 | 0.5 | 0.38461538 | 0.6 | colon.bma.model.cluster.Resi- |
| 0.0013 | alt.recessive.9:117568766, | | | | | | | | | | dent.macrophages.RDS |
| 0 | alt.negative.9:117568766, | | | | | | | | | | |
| 0.00134 | ref.recessive.21:45658474, | | | | | | | | | | |
| 7e−05 | ref.negative.21:45658474, | | | | | | | | | | |
| 0.00147 | ref.negative.5:11562221, | | | | | | | | | | |
| 0.00011 | ref.negative.14:98734567, | | | | | | | | | | |
| 7e−04 | alt.negative.16:11331509, | | | | | | | | | | |
| 0.00019 | ref.negative.16:11331509 | | | | | | | | | | |
| 6e−05 | additive.9:117568766, | 5 | 0.39215686 | 0.49019608 | 0.50615385 | 0.5 | 0.48387097 | 0.5 | 0.38461538 | 0.6 | colon.bma.model.cluster.Resi- |
| 0.00117 | alt.recessive.9:117568766, | | | | | | | | | | dent.macrophages.RDS |
| 0 | alt.negative.9:117568766, | | | | | | | | | | |
| 0.0014 | ref.recessive.21:45658474, | | | | | | | | | | |
| 2e−04 | refnegative.21:45658474, | | | | | | | | | | |
| 0.00112 | ref.negative.5:11562221, | | | | | | | | | | |
| 0.00013 | ref.negative.14:98734567, | | | | | | | | | | |
| 0.00094 | ref.negative.16:6147645 | | | | | | | | | | |
| 0.00021 | additive.9:117568766, | 5 | 0.31372549 | 0.49019608 | 0.49076923 | 0.5 | 0.48571429 | 0.5 | 0.30769231 | 0.68 | colon.bma.model.cluster.Resi- |
| 0.00107 | alt.reccssive.9:117568766, | | | | | | | | | | dent.macrophages.RDS |
| 0 | alt.negative.9:117568766, | | | | | | | | | | |
| 0.00168 | ref.recessive.21:45658474, | | | | | | | | | | |
| 0.00018 | ref.negative.21:45658474, | | | | | | | | | | |
| 0.00126 | ref.negative.5:11562221, | | | | | | | | | | |
| 0.00035 | alt.negative.16:11331509, | | | | | | | | | | |
| −5e−05 | ref.negative.16:11331509, | | | | | | | | | | |
| 0.00095 | ref.negative.16:6147645 | | | | | | | | | | |
| 0.00022 | additive.9:117568766, | 5 | 0.35294118 | 0.49019608 | 0.51 | 0.5 | 0.48484848 | 0.5 | 0.34615385 | 0.64 | colon.bma.model.cluster.Resi- |
| 0.00107 | alt.recessive.9:117568766, | | | | | | | | | | dent.macrophages.RDS |
| 0 | alt.negative.9:117568766, | | | | | | | | | | |
| 0.00145 | ref.recessive.21:45658474, | | | | | | | | | | |
| 0.00032 | ref.negative.21:45658474, | | | | | | | | | | |
| 0.00151 | ref.negative.20:62305274, | | | | | | | | | | |
| 0.00071 | ref.negative.14:98734567, | | | | | | | | | | |
| 0.00109 | ref.negative.16:6147645 | | | | | | | | | | |
| 0.00039 | additive.9:117568766, | 5 | 0.31372549 | 0.49019608 | 0.51230769 | 0.5 | 0.48571429 | 0.5 | 0.30769231 | 0.68 | colon.bma.model.cluster.Resi- |
| 0.00075 | alt.recessive.9:117568766, | | | | | | | | | | dent.macrophages.RDS |
| 0 | alt.negative.9:117568766, | | | | | | | | | | |
| 0.00128 | ref.recessive.21:45658474, | | | | | | | | | | |
| 0.00054 | refnegative.21:45658474, | | | | | | | | | | |
| 0.00134 | ref.negative.20:62305274, | | | | | | | | | | |
| 0.00056 | alt.negative.16:11331509, | | | | | | | | | | |
| 0.00017 | ref.negative.16:11331509, | | | | | | | | | | |
| 0.00089 | ref.negative.16:6147645 | | | | | | | | | | |
| 0.00046 | additive.9:117568766, | 5 | 0.47058824 | 0.49019608 | 0.52230769 | 0.5 | 0.48148148 | 0.5 | 0.46153846 | 0.52 | colon.bma.model.cluster.Resi- |
| 0.00096 | alt.recessive.9:117568766, | | | | | | | | | | dent.macrophages.RDS |
| 0 | alt.negative.9:117568766, | | | | | | | | | | |
| 0.00104 | ref.negative.5:11562221, | | | | | | | | | | |
| 0.00147 | ref.negative.20:62305274, | | | | | | | | | | |
| 0.00063 | ref.negative.14:98734567, | | | | | | | | | | |
| 0.00061 | alt.negative.16:11331509, | | | | | | | | | | |
| 0.00011 | ref.negative.16:11331509 | | | | | | | | | | |
| 0.00076 | additive.9:117568766, | 5 | 0.47058824 | 0.49019608 | 0.52846154 | 0.5 | 0.48148148 | 0.5 | 0.46153846 | 0.52 | colon.bma.model.cluster.Resi- |
| 0.00064 | alt.recessive.9:117568766, | | | | | | | | | | dent.macrophages.RDS |
| 0 | alt.negative.9:117568766, | | | | | | | | | | |
| 0.00092 | ref.negative.5:11562221, | | | | | | | | | | |
| 0.00152 | ref.negative.20:62305274, | | | | | | | | | | |
| 0.00091 | ref.negative.14:98734567, | | | | | | | | | | |
| 0.00078 | ref.negative.16:6147645 | | | | | | | | | | |
| 0.00074 | additive.9:117568766, | 5 | 0.35294118 | 0.49019608 | 0.46769231 | 0.5 | 0.48484848 | 0.5 | 0.34615385 | 0.64 | colon.bma.model.cluster.Resi- |
| 0.00057 | alt.recessive.9:117568766, | | | | | | | | | | dent.macrophages.RDS |
| 0 | alt.negative.9:117568766, | | | | | | | | | | |
| 0.00093 | ref.negative.5:11562221, | | | | | | | | | | |
| 0.00065 | ref.negative.14:98734567, | | | | | | | | | | |
| 0.00069 | alt.negative.16:11331509, | | | | | | | | | | |
| 6e−05 | ref.negative.16:11331509, | | | | | | | | | | |
| 0.00075 | ref.negative.16:6147645 | | | | | | | | | | |
| 0.00155 | ref.recessive.21:45658474, | 5 | 0.35294118 | 0.49019608 | 0.48846154 | 0.5 | 0.48484848 | 0.5 | 0.34615385 | 0.64 | colon.bma.model.cluster.Resi- |
| 0.00088 | ref.negative.21:45658474, | | | | | | | | | | dent.macrophages.RDS |
| 0.00079 | ref.negative.20:62305274, | | | | | | | | | | |
| 0.00036 | ref.negative.14:98734567, | | | | | | | | | | |
| 0.00062 | alt.negative.16:11331509, | | | | | | | | | | |
| 0.00043 | ref.negative.16:11331509, | | | | | | | | | | |
| 0.00074 | ref.negative.16:6147645 | | | | | | | | | | |
| 0.00172 | ref.negative.5:11562221, | 5 | 0.35294118 | 0.49019608 | 0.51230769 | 0.5 | 0.48484848 | 0.5 | 0.34615385 | 0.64 | colon.bma.model.cluster.Resi- |
| 0.00073 | ref.negative.20:62305274, | | | | | | | | | | dent.macrophages.RDS |
| 0.00081 | ref.negative.11:128443099, | | | | | | | | | | |
| 0.00075 | alt.negative.16:11331509, | | | | | | | | | | |
| 0.00043 | ref.negative.16:11331509, | | | | | | | | | | |
| 0.00073 | ref.negative.16:6147645 | | | | | | | | | | |
| 0.00135 | ref.negative.5:11562221, | 5 | 0.31372549 | 0.49019608 | 0.48076923 | 0.5 | 0.48571429 | 0.5 | 0.30769231 | 0.68 | colon.bma.model.cluster.Resi- |
| 0.00068 | ref.negative.11:128443099, | | | | | | | | | | dent.macrophages.RDS |
| 0.00049 | ref.negative.14:98734567, | | | | | | | | | | |
| 0.00069 | alt.negative.16:11331509, | | | | | | | | | | |
| 0.00023 | ref.negative.16:11331509, | | | | | | | | | | |
| 0.00073 | ref.negative.16:6147645 | | | | | | | | | | |
| 0.00064 | additive.9:117568766, | 5 | 0.49019608 | 0.47058824 | 0.49076923 | 0.48 | 0.46153846 | 0.48 | 0.46153846 | 0.48 | colon.bma.model.cluster.Resi- |
| 0.00087 | alt.recessive.9:117568766, | | | | | | | | | | dent.macrophages.RDS |
| 0 | alt.negative.9:117568766, | | | | | | | | | | |
| 0.00071 | ref.negative.5:11562221, | | | | | | | | | | |
| 0.00112 | ref.negative.20:62305274, | | | | | | | | | | |
| 0.00076 | ref.negative.11:128443099, | | | | | | | | | | |
| 0.00099 | ref.negative.16:6147645 | | | | | | | | | | |
| 0.00064 | additive.9:117568766, | 5 | 0.45098039 | 0.47058824 | 0.50153846 | 0.47826087 | 0.46428571 | 0.47826087 | 0.42307692 | 0.52 | colon.bma.model.cluster.Resi- |
| 0.00068 | alt.recessive.9:117568766, | | | | | | | | | | dent.macrophages.RDS |
| 0 | alt.negative.9:117568766, | | | | | | | | | | |
| 0.00088 | ref.negative.5:11562221, | | | | | | | | | | |
| 0.00089 | ref.negative.11:128443099, | | | | | | | | | | |
| 0.00019 | ref.negative.14:98734567, | | | | | | | | | | |
| 5e−04 | alt.negative.16:11331509, | | | | | | | | | | |
| −6e−05 | ref.negative.16:11331509 | | | | | | | | | | |
| 0.00064 | additive.9:117568766, | 5 | 0.45098039 | 0.47058824 | 0.50846154 | 0.47826087 | 0.46428571 | 0.47826087 | 0.42307692 | 0.52 | colon.bma.model.cluster.Resi- |
| 0.00091 | alt.recessive.9:117568766, | | | | | | | | | | dent.macrophages.RDS |
| 0 | alt.negative.9:117568766, | | | | | | | | | | |
| 0.00106 | ref.negative.20:62305274, | | | | | | | | | | |
| 0.00086 | ref.negative.11:128443099, | | | | | | | | | | |
| 0.00101 | ref.negative.14:98734567, | | | | | | | | | | |
| 0.00094 | ref.negative.16:6147645 | | | | | | | | | | |
| 0.00067 | additive.9:117568766, | 5 | 0.37254902 | 0.47058824 | 0.49538462 | 0.47368421 | 0.46875 | 0.47368421 | 0.34615385 | 0.6 | colon.bma.model.cluster.Resi- |
| 0.00057 | alt.recessive.9:117568766, | | | | | | | | | | dent.macrophages.RDS |
| 0 | alt.negative.9:117568766, | | | | | | | | | | |
| 0.0011 | ref.negative.5:11562221, | | | | | | | | | | |
| 0.00147 | ref.negative.20:62305274, | | | | | | | | | | |
| 0.00052 | alt.negative.16:11331509, | | | | | | | | | | |
| 0.00013 | ref.negative.16:11331509, | | | | | | | | | | |
| 0.00063 | ref.negative.16:6147645 | | | | | | | | | | |
| 0.00168 | ref.recessive.21:45658474, | 5 | 0.33333333 | 0.47058824 | 0.48615385 | 0.47058824 | 0.47058824 | 0.47058824 | 0.30769231 | 0.64 | colon.bma.model.cluster.Resi- |
| 0.00097 | ref.negative.21:45658474, | | | | | | | | | | dent.macrophages.RDS |
| 0.00093 | ref.negative.11:128443099, | | | | | | | | | | |
| 5e−04 | ref.negative.14:98734567, | | | | | | | | | | |
| 0.00023 | alt.negative.16:11331509, | | | | | | | | | | |
| 0.00024 | ref.negative.16:11331509, | | | | | | | | | | |
| 0.00096 | ref.negative.16:6147645 | | | | | | | | | | |
| 0.00071 | additive.9:117568766, | 5 | 0.43137255 | 0.45098039 | 0.51307692 | 0.45454545 | 0.44827586 | 0.45454545 | 0.38461538 | 0.52 | colon.bma.model.cluster.Resi- |
| 0.00043 | alt.recessive.9:117568766, | | | | | | | | | | dent.macrophages.RDS |
| 0 | alt.negative.9:117568766, | | | | | | | | | | |
| 0.00102 | ref.negative.5:11562221, | | | | | | | | | | |
| 0.00087 | ref.negative.20:62305274, | | | | | | | | | | |
| 0.00066 | ref.negative.11:128443099, | | | | | | | | | | |
| 0.00057 | alt.negative.16:11331509, | | | | | | | | | | |
| 2e−05 | ref.negative.16:11331509 | | | | | | | | | | |
| 7e−04 | additive.9:117568766, | 5 | 0.43137255 | 0.45098039 | 0.49769231 | 0.45454545 | 0.44827586 | 0.45454545 | 0.38461538 | 0.52 | colon.bma.model.cluster.Resi- |
| 0.00085 | alt.recessive.9:117568766, | | | | | | | | | | dent.macrophages.RDS |
| 0 | alt.negative.9:117568766, | | | | | | | | | | |
| 8e−04 | ref.negative.5:11562221, | | | | | | | | | | |
| 0.00105 | ref.negative.11:128443099, | | | | | | | | | | |
| 0.00082 | ref.negative.14:98734567, | | | | | | | | | | |
| 0.00099 | ref.negative.16:6147645 | | | | | | | | | | |
| 0.00062 | additive.9:117568766, | 5 | 0.43137255 | 0.45098039 | 0.50230769 | 0.45454545 | 0.44827586 | 0.45454545 | 0.38461538 | 0.52 | colon.bma.model.cluster.Resi- |
| 0.00085 | alt.recessive.9:117568766, | | | | | | | | | | dent.macrophages.RDS |
| 0 | alt.negative.9:117568766, | | | | | | | | | | |
| 0.00104 | ref.negative.20:62305274, | | | | | | | | | | |
| 0.00082 | ref.negative.11:128443099, | | | | | | | | | | |
| 0.00034 | ref.negative.14:98734567, | | | | | | | | | | |
| 0.00047 | alt.negative.16:11331509, | | | | | | | | | | |
| 9e−05 | ref.negative.16:11331509 | | | | | | | | | | |
| 0.00155 | ref.recessive.21:45658474, | 5 | 0.39215686 | 0.45098039 | 0.47615385 | 0.45 | 0.4516129 | 0.45 | 0.34615385 | 0.56 | colon.bma.model.cluster.Resi- |
| 0.00068 | ref.negative.21:45658474, | | | | | | | | | | dent.macrophages.RDS |
| 0.00157 | ref.negative.5:11562221, | | | | | | | | | | |
| 0.00068 | ref.negative.14:98734567, | | | | | | | | | | |
| 0.00063 | alt.negative.16:11331509, | | | | | | | | | | |
| 4e−04 | ref.negative.16:11331509, | | | | | | | | | | |
| 0.00098 | ref.negative.16:6147645 | | | | | | | | | | |
| 0.00165 | ref.negative.5:11562221, | 5 | 0.39215686 | 0.45098039 | 0.54615385 | 0.45 | 0.4516129 | 0.45 | 0.34615385 | 0.56 | colon.bma.model.cluster.Resi- |
| 0.00073 | ref.negative.20:62305274, | | | | | | | | | | dent.macrophages.RDS |
| 0.00068 | ref.negative.11:128443099, | | | | | | | | | | |
| 0.00034 | ref.negative.14:98734567, | | | | | | | | | | |
| 0.00061 | alt.negative.16:11331509, | | | | | | | | | | |
| 0.00033 | ref.negative.16:11331509 | | | | | | | | | | |
| 0.00046 | additive.9:117568766, | 5 | 0.31372549 | 0.45098039 | 0.48769231 | 0.4375 | 0.45714286 | 0.4375 | 0.26923077 | 0.64 | colon.bma.model.cluster.Resi- |
| 0.00053 | alt.recessive.9:117568766, | | | | | | | | | | dent.macrophages.RDS |
| 0 | alt.negative.9:117568766, | | | | | | | | | | |
| 0.00119 | ref.recessive.21:45658474, | | | | | | | | | | |
| 0.00049 | ref.negative.21:45658474, | | | | | | | | | | |
| 2e−04 | ref.negative.14:98734567, | | | | | | | | | | |
| 0.00041 | alt.negative.16:11331509, | | | | | | | | | | |
| −0.00022 | ref.negative.16:11331509, | | | | | | | | | | |
| 0.00081 | ref.negative.16:6147645 | | | | | | | | | | |
| 0.00127 | ref.negative.5:11562221, | 5 | 0.31372549 | 0.45098039 | 0.51692308 | 0.4375 | 0.45714286 | 0.4375 | 0.26923077 | 0.64 | colon.bma.model.cluster.Resi- |
| 0.00052 | ref.negative.20:62305274, | | | | | | | | | | dent.macrophages.RDS |
| 0.00071 | ref.negative.11:128443099, | | | | | | | | | | |
| 4e−04 | ref.negative.14:98734567, | | | | | | | | | | |
| 0.00067 | ref.negative.16:6147645 | | | | | | | | | | |
| 0.00061 | ref.negative.20:62305274, | 5 | 0.31372549 | 0.45098039 | 0.48846154 | 0.4375 | 0.45714286 | 0.4375 | 0.26923077 | 0.64 | colon.bma.model.cluster.Resi- |
| 0.00083 | ref.negative.11:128443099, | | | | | | | | | | dent.macrophages.RDS |
| 0.00042 | ref.negative.14:98734567, | | | | | | | | | | |
| 0.00048 | alt.negative.16:11331509, | | | | | | | | | | |
| 0.00021 | ref.negative.16:11331509, | | | | | | | | | | |
| 0.00064 | ref.negative.16:6147645 | | | | | | | | | | |
| 0.00041 | additive.9:117568766, | 5 | 0.41176471 | 0.43137255 | 0.53076923 | 0.42857143 | 0.43333333 | 0.42857143 | 0.34615385 | 0.52 | colon.bma.model.cluster.Resi- |
| 0.00104 | alt.recessive.9:117568766, | | | | | | | | | | dent.macrophages.RDS |
| 0 | alt.negative.9:117568766, | | | | | | | | | | |
| 0.00081 | ref.negative.5:11562221, | | | | | | | | | | |
| 0.00111 | ref.negative.20:62305274, | | | | | | | | | | |
| 0.00073 | ref.negative.11:128443099, | | | | | | | | | | |
| 0.00025 | ref.negative.14:98734567 | | | | | | | | | | |
| 0.00078 | additive.9:117568766, | 5 | 0.37254902 | 0.43137255 | 0.48153846 | 0.42105263 | 0.4375 | 0.42105263 | 0.30769231 | 0.56 | colon.bma.model.cluster.Resi- |
| 6e−04 | alt.recessive.9:117568766, | | | | | | | | | | dent.macrophages.RDS |
| 0 | alt.negative.9:117568766, | | | | | | | | | | |
| 0.00093 | ref.negative.5:11562221, | | | | | | | | | | |
| 9e−04 | ref.negative.11:128443099, | | | | | | | | | | |
| 0.00064 | alt.negative.16:11331509, | | | | | | | | | | |
| −3e−05 | ref.negative.16:11331509, | | | | | | | | | | |
| 8e−04 | ref.negative.16:6147645 | | | | | | | | | | |
| 0.00093 | additive.9:117568766, | 5 | 0.37254902 | 0.43137255 | 0.46692308 | 0.42105263 | 0.4375 | 0.42105263 | 0.30769231 | 0.56 | colon.bma.model.cluster.Resi- |
| 0.00052 | alt.recessive.9:117568766, | | | | | | | | | | dent.macrophages.RDS |
| 0 | alt.negative.9:117568766, | | | | | | | | | | |
| 0.00093 | ref.negative.20:62305274, | | | | | | | | | | |
| 0.00092 | ref.negative.11:128443099, | | | | | | | | | | |
| 0.00046 | alt.negative.16:11331509, | | | | | | | | | | |
| −0.00028 | ref.negative.16:11331509, | | | | | | | | | | |
| 0.00073 | ref.negative.16:6147645 | | | | | | | | | | |
| 0.00074 | additive.9:117568766, | 5 | 0.37254902 | 0.43137255 | 0.47846154 | 0.42105263 | 0.4375 | 0.42105263 | 0.30769231 | 0.56 | colon.bma.model.cluster.Resi- |
| 0.00058 | alt.recessive.9:117568766, | | | | | | | | | | dent.macrophages.RDS |
| 0 | alt.negative.9:117568766, | | | | | | | | | | |
| 0.00137 | ref.negative.20:62305274, | | | | | | | | | | |
| 0.00071 | ref.negative.14:98734567, | | | | | | | | | | |
| 0.00035 | alt.negative.16:11331509, | | | | | | | | | | |
| −0.00029 | ref.negative.16:11331509, | | | | | | | | | | |
| 0.00073 | ref.negative.16:6147645 | | | | | | | | | | |
| 0.00091 | additive.9:117568766, | 5 | 0.35294118 | 0.41176471 | 0.47846154 | 0.38888889 | 0.42424242 | 0.38888889 | 0.26923077 | 0.56 | colon.bma.model.cluster.Resi- |
| 0.00045 | alt.recessive.9:117568766, | | | | | | | | | | dent.macrophages.RDS |
| 0 | alt.negative.9:117568766, | | | | | | | | | | |
| 0.00098 | ref.negative.11:128443099, | | | | | | | | | | |
| 0.00043 | ref.negative.14:98734567, | | | | | | | | | | |
| 3e−04 | alt.negative.16:11331509, | | | | | | | | | | |
| −0.00021 | ref.negative.16:11331509, | | | | | | | | | | |
| 0.00075 | ref.negative.16:6147645 | | | | | | | | | | |
| 0.00023 | additive.9:117568766, | 4 | 0.37254902 | 0.58823529 | 0.52307692 | 0.63157895 | 0.5625 | 0.63157895 | 0.46153846 | 0.72 | colon.bma.model.cluster.Resi- |
| 0.00105 | alt.recessive.9:117568766, | | | | | | | | | | dent.macrophages.RDS |
| 0 | alt.negative.9:117568766, | | | | | | | | | | |
| 0.00151 | ref.recessive.21:45658474, | | | | | | | | | | |
| 0.00049 | refnegative.21:45658474, | | | | | | | | | | |
| 0.00115 | ref.negative.11:128443099, | | | | | | | | | | |
| 0.00109 | ref.negative.16:6147645 | | | | | | | | | | |
| 0.00157 | ref.recessive.21:45658474, | 4 | 0.31372549 | 0.56862745 | 0.51461538 | 0.625 | 0.5285714 | 0.625 | 0.38461538 | 0.76 | colon.bma.model.cluster.Resi- |
| 0.00054 | ref.negative.21:45658474, | | | | | | | | | | dent.macrophages.RDS |
| 0.00126 | ref.negative.5:11562221, | | | | | | | | | | |
| 0.00061 | ref.negative.20:62305274, | | | | | | | | | | |
| 0.00083 | ref.negative.11:128443099 | | | | | | | | | | |
| 0.00162 | ref.recessive.21:45658474, | 4 | 0.31372549 | 0.56862745 | 0.52153846 | 0.625 | 0.54285714 | 0.625 | 0.38461538 | 0.76 | colon.bma.model.cluster.Resi- |
| 0.00062 | ref.negative.21:45658474, | | | | | | | | | | dent.macrophages.RDS |
| 0.00142 | ref.negative.5:11562221, | | | | | | | | | | |
| 0.00105 | ref.negative.11:128443099, | | | | | | | | | | |
| 0.00018 | ref.negative.14:98734567 | | | | | | | | | | |
| 0.00136 | ref.recessive.21:45658474, | 4 | 0.31372549 | 0.56862745 | 0.53153846 | 0.625 | 0.54285714 | 0.625 | 0.38461538 | 0.76 | colon.bma.model.cluster.Resi- |
| 9e−04 | ref.negative.21:45658474, | | | | | | | | | | dent.macrophages.RDS |
| 0.00135 | ref.negative.5:11562221, | | | | | | | | | | |
| 0.00085 | ref.negative.11:128443099, | | | | | | | | | | |
| 8e−04 | alt.negative.16:11331509, | | | | | | | | | | |
| 0.00065 | ref.negative.16:11331509 | | | | | | | | | | |
| −0.00012 | additive.9:117568766, | 4 | 0.35294118 | 0.56862745 | 0.54153846 | 0.61111111 | 0.54545455 | 0.61111111 | 0.42307692 | 0.72 | colon.bma.model.cluster.Resi- |
| 0.00129 | alt.recessive.9:117568766, | | | | | | | | | | dent.macrophages.RDS |
| 0 | alt.negative.9:117568766, | | | | | | | | | | |
| 0.00137 | ref.recessive.21:45658474, | | | | | | | | | | |
| 0.00035 | ref.negative.21:45658474, | | | | | | | | | | |
| 0.00105 | ref.negative.5:11562221, | | | | | | | | | | |
| 0.0011 | ref.negative.11:128443099 | | | | | | | | | | |
| 0.00149 | ref.recessive.21:45658474, | 4 | 0.33333333 | 0.54901961 | 0.53 | 0.58823529 | 0.52941176 | 0.58823529 | 0.38461538 | 0.72 | colon.bma.model.cluster.Resi- |
| 0.00103 | ref.negative.21:45658474, | | | | | | | | | | dent.macrophages.RDS |
| 0.00063 | ref.negative.20:62305274, | | | | | | | | | | |
| 0.00087 | ref.negative.11:128443099, | | | | | | | | | | |
| 0.00056 | alt.negative.16:11331509, | | | | | | | | | | |
| 0.00056 | ref.negative.16:11331509 | | | | | | | | | | |
| 0.00142 | ref.recessive.21:45658474, | 4 | 0.33333333 | 0.54901961 | 0.51923077 | 0.58823529 | 0.52941176 | 0.58823529 | 0.38461538 | 0.72 | colon.bma.model.cluster.Resi- |
| 0.00095 | ref.negative.21:45658474, | | | | | | | | | | dent.macrophages.RDS |
| 0.00027 | ref.negative.20:62305274, | | | | | | | | | | |
| 0.00092 | ref.negative.11:128443099, | | | | | | | | | | |
| 0.00077 | ref.negative.16:6147645 | | | | | | | | | | |
| 0.00148 | ref.recessive.21:45658474, | 4 | 0.33333333 | 0.54901961 | 0.51615385 | 0.58823529 | 0.52941176 | 0.58823529 | 0.38461538 | 0.72 | colon.bma.model.cluster.Resi- |
| 0.00087 | ref.negative.21:45658474, | | | | | | | | | | dent.macrophages.RDS |
| 0.00101 | ref.negative.11:128443099, | | | | | | | | | | |
| −1e−05 | ref.negative.14:98734567, | | | | | | | | | | |
| 0.00048 | alt.negative.16:11331509, | | | | | | | | | | |
| 0.00053 | ref.negative.16:11331509 | | | | | | | | | | |
| 8e−05 | additive.9:117568766, | 4 | 0.37254902 | 0.54901961 | 0.54153846 | 0.57894737 | 0.53125 | 0.57894737 | 0.42307692 | 0.68 | colon.bma.model.cluster.Resi- |
| 0.00116 | alt.recessive.9:117568766, | | | | | | | | | | dent.macrophages.RDS |
| 0 | alt.negative.9:117568766, | | | | | | | | | | |
| 0.00144 | ref.recessive.21:45658474, | | | | | | | | | | |
| 0.00042 | ref.negative.21:45658474, | | | | | | | | | | |
| 0.00073 | ref.negative.20:62305274, | | | | | | | | | | |
| 0.00105 | ref.negative.11:128443099 | | | | | | | | | | |
| −6e−05 | additive.9:117568766, | 4 | 0.37254902 | 0.54901961 | 0.52615385 | 0.57894737 | 0.53125 | 0.57894737 | 0.42307692 | 0.68 | colon.bma.model.cluster.Resi- |
| 0.00134 | alt.recessive.9:117568766, | | | | | | | | | | dent.macrophages.RDS |
| 0 | alt.negative.9:117568766, | | | | | | | | | | |
| 0.00146 | ref.recessive.21:45658474, | | | | | | | | | | |
| 0.00041 | ref.negative.21:45658474, | | | | | | | | | | |
| 0.00111 | ref.negative.11:128443099, | | | | | | | | | | |
| −0.00019 | ref.negative.14:98734567 | | | | | | | | | | |
| 0.00148 | ref.recessive.21:45658474, | 4 | 0.41176471 | 0.54901961 | 0.54230769 | 0.57142857 | 0.53333333 | 0.57142857 | 0.46153846 | 0.64 | colon.bma.model.cluster.Resi- |
| 0.00047 | ref.negative.21:45658474, | | | | | | | | | | dent.macrophages.RDS |
| 0.00128 | ref.negative.5:11562221, | | | | | | | | | | |
| 0.00116 | ref.negative.20:62305274, | | | | | | | | | | |
| 0.00013 | ref.negative.14:98734567 | | | | | | | | | | |
| −0.00021 | additive.9:117568766, | 4 | 0,45098039 | 0.54901961 | 0.54538462 | 0.56521739 | 0.53571429 | 0.56521739 | 0.5 | 0.6 | colon.bma.model.cluster.Resi- |
| 0.00155 | alt.recessive.9:117568766, | | | | | | | | | | dent.macrophages.RDS |
| 0 | alt.negative.9:117568766, | | | | | | | | | | |
| 0.00154 | ref.recessive.21:45658474, | | | | | | | | | | |
| 1e−05 | ref.negative.21:45658474, | | | | | | | | | | |
| 0.00141 | ref.negative.5:11562221, | | | | | | | | | | |
| 0.0014 | ref.negative.20:62305274 | | | | | | | | | | |
| 0.00164 | ref.recessive.21:45658474, | 4 | 0.45098039 | 0.54901961 | 0.51615385 | 0.56521739 | 0.53571429 | 0.56521739 | 0.5 | 0.6 | colon.bma.model.cluster.Resi- |
| 0.00049 | ref.negative.21:45658474, | | | | | | | | | | dent.macrophages.RDS |
| 0.00165 | ref.negative.5:11562221, | | | | | | | | | | |
| 0.00119 | ref.negative.20:62305274, | | | | | | | | | | |
| 0.00059 | alt.negative.16:11331509, | | | | | | | | | | |
| 0.00032 | ref.negative.16:11331509 | | | | | | | | | | |
| 0.00149 | ref.recessive.21:45658474, | 4 | 0.45098039 | 0.54901961 | 0.49846154 | 0.56521739 | 0.53571429 | 0.56521739 | 0.5 | 0.6 | colon.bma.model.cluster.Resi- |
| 0.00047 | ref.negative.21:45658474, | | | | | | | | | | dent.macrophages.RDS |
| 0.00135 | ref.negative.5:11562221, | | | | | | | | | | |
| 0.00096 | ref.negative.20:62305274, | | | | | | | | | | |
| 0.00083 | ref.negative.16:6147645 | | | | | | | | | | |
| 0.00022 | additive.9:117568766, | 4 | 0.31372549 | 0.52941176 | 0.52615385 | 0.5625 | 0.51428571 | 0.5625 | 0.34615385 | 0.72 | colon.bma.model.cluster.Resi- |
| 0.00098 | alt.recessive.9:117568766, | | | | | | | | | | dent.macrophages.RDS |
| 0 | alt.negative.9:117568766, | | | | | | | | | | |
| 0.00135 | ref.recessive.21:45658474, | | | | | | | | | | |
| 0.00048 | ref.negative.21:45658474, | | | | | | | | | | |
| 0.00109 | ref.negative.11:128443099, | | | | | | | | | | |
| 0.00027 | alt.negative.16:11331509, | | | | | | | | | | |
| 1e−04 | ref.negative.16:11331509 | | | | | | | | | | |
| 0.00159 | ref.recessive.21:45658474, | 4 | 0.31372549 | 0.52941176 | 0.51076923 | 0.5625 | 0.51428571 | 0.5625 | 0.34615385 | 0.72 | colon.bma.model.cluster.Resi- |
| 0.00078 | ref.negative.21:45658474, | | | | | | | | | | dent.macrophages.RDS |
| 0.00039 | ref.negative.20:62305274, | | | | | | | | | | |
| 0.00096 | ref.negative.11:128443099, | | | | | | | | | | |
| 5e−05 | ref.negative.14:98734567 | | | | | | | | | | |
| 0.00141 | ref.recessive.21:45658474, | 4 | 0.31372549 | 0.52941176 | 0.52923077 | 0.5625 | 0.51428571 | 0.5625 | 0.34615385 | 0.72 | colon.bma.model.cluster.Resi- |
| 9e−04 | ref.negative.21:45658474, | | | | | | | | | | dent.macrophages.RDS |
| 0.00095 | ref.negative.20:62305274, | | | | | | | | | | |
| 3e−04 | ref.negative.14:98734567, | | | | | | | | | | |
| 0.00066 | alt.negative.16:11331509, | | | | | | | | | | |
| 5e−04 | ref.negative.16:11331509 | | | | | | | | | | |
| 0.00164 | ref.negative.5:11562221, | 4 | 0.39215686 | 0.52941176 | 0.55461538 | 0.55 | 0.51612903 | 0.55 | 0.42307692 | 0.64 | colon.bma.model.cluster.Resi- |
| 0.00106 | ref.negative.20:62305274, | | | | | | | | | | dent.macrophages.RDS |
| 0.00043 | ref.negative.14:98734567, | | | | | | | | | | |
| 0.00065 | alt.negative.16:11331509, | | | | | | | | | | |
| 0.00039 | ref.negative.16:11331509 | | | | | | | | | | |
| 9e−05 | additive.9:117568766, | 4 | 0.47058824 | 0.52941176 | 0.54307692 | 0.54166667 | 0.51851852 | 0.54166667 | 0.5 | 0.56 | colon.bma.model.cluster.Resi- |
| 0.0012 | alt.recessive.9:117568766, | | | | | | | | | | dent.macrophages.RDS |
| 0 | alt.negative.9:117568766, | | | | | | | | | | |
| 0.0014 | ref.recessive.21:45658474, | | | | | | | | | | |
| 0.00024 | ref.negative.21:45658474, | | | | | | | | | | |
| 0.00144 | ref.negative.20:62305274, | | | | | | | | | | |
| 9e−05 | ref.negative.14:98734567 | | | | | | | | | | |
| 0.00125 | ref.negative.5:11562221, | 4 | 0.58823529 | 0.52941176 | 0.56384615 | 0.53333333 | 0.52380952 | 0.53333333 | 0.61538462 | 0.44 | colon.bma.model.cluster.Resi- |
| 0.00074 | ref.negative.20:62305274, | | | | | | | | | | dent.macrophages.RDS |
| 0.00066 | ref.negative.11:128443099, | | | | | | | | | | |
| 7e−05 | ref.negative.14:98734567 | | | | | | | | | | |
| 0.00151 | ref.recessive.21:45658474, | 4 | 0.33333333 | 0.50980392 | 0.49 | 0.52941176 | 0.5 | 0.52941176 | 0.34615385 | 0.68 | colon.bma.model.cluster.Resi- |
| 0.00058 | ref.negative.21:45658474, | | | | | | | | | | dent.macrophages.RDS |
| 0.00184 | ref.negative.5:11562221, | | | | | | | | | | |
| 1e−04 | ref.negative.14:98734567, | | | | | | | | | | |
| 9e−04 | alt.negative.16:11331509, | | | | | | | | | | |
| 0.00079 | ref.negative.16:11331509 | | | | | | | | | | |
| 0.00023 | additive.9:117568766, | 4 | 0.37254902 | 0.50980392 | 0.51384615 | 0.52631579 | 0.5 | 0.52631579 | 0.38461538 | 0.64 | colon.bma.model.cluster.Resi- |
| 0.00102 | alt.recessive.9:117568766, | | | | | | | | | | dent.macrophages.RDS |
| 0 | alt.negative.9:117568766, | | | | | | | | | | |
| 0.00133 | ref.recessive.21:45658474, | | | | | | | | | | |
| 0.00035 | ref.negative.21:45658474, | | | | | | | | | | |
| 0.00142 | ref.negative.20:62305274, | | | | | | | | | | |
| 0.00063 | alt.negative.16:11331509, | | | | | | | | | | |
| 0.00025 | ref.negative.16:11331509 | | | | | | | | | | |
| 0.00025 | additive.9:117568766, | 4 | 0.37254902 | 0.50980392 | 0.50692308 | 0.52631579 | 0.5 | 0.52631579 | 0.38461538 | 0.64 | colon.bma.model.cluster.Resi- |
| 0.001 | alt.recessive.9:117568766, | | | | | | | | | | dent.macrophages.RDS |
| 0 | alt.negative.9:117568766, | | | | | | | | | | |
| 0.00138 | ref.recessive.21:45658474, | | | | | | | | | | |
| 0.00039 | ref.negative.21:45658474, | | | | | | | | | | |
| 0.00129 | ref.negative.20:62305274, | | | | | | | | | | |
| 0.00089 | ref.negative.16:6147645 | | | | | | | | | | |
| 0.00144 | ref.negative.5:11562221, | 4 | 0.41176471 | 0.50980392 | 0.56692308 | 0.52380952 | 0.5 | 0.52380952 | 0.42307692 | 0.6 | colon.bma.model.cluster.Resi- |
| 0.00098 | ref.negative.20:62305274, | | | | | | | | | | dent.macrophages.RDS |
| 0.00084 | ref.negative.14:98734567, | | | | | | | | | | |
| 0.00073 | ref.negative.16:6147645 | | | | | | | | | | |
| 0.00143 | ref.recessive.21:45658474, | 4 | 0.45098039 | 0.50980392 | 0.48692308 | 0.52173913 | 0.5 | 0.52173913 | 0.46153846 | 0.56 | colon.bma.model.cluster.Resi- |
| 0.00085 | ref.negative.21:45658474, | | | | | | | | | | dent.macrophages.RDS |
| 0.00104 | ref.negative.20:62305274, | | | | | | | | | | |
| 0.00055 | ref.negative.14:98734567, | | | | | | | | | | |
| 0.00093 | ref.negative.16:6147645 | | | | | | | | | | |
| 0.00161 | ref.negative.5:11562221, | 4 | 0.49019608 | 0.50980392 | 0.50307692 | 0.52 | 0.5 | 0.52 | 0.5 | 0.52 | colon.bma.model.cluster.Resi- |
| 0.00104 | ref.negative.20:62305274, | | | | | | | | | | dent.macrophages.RDS |
| 0.00077 | alt.negative.16:11331509, | | | | | | | | | | |
| 0.00043 | ref.negative.16:11331509, | | | | | | | | | | |
| 6e−04 | ref.negative.16:6147645 | | | | | | | | | | |
| −0.00017 | additive.9:117568766, | 4 | 0.50980392 | 0.49019608 | 0.51538462 | 0.5 | 0.48 | 0.5 | 0.5 | 0.48 | colon.bma.model.cluster.Resi- |
| 0.00135 | alt.recessive.9:117568766, | | | | | | | | | | dent.macrophages.RDS |
| 0 | alt.negative.9:117568766, | | | | | | | | | | |
| 0.00132 | ref.recessive.21:45658474, | | | | | | | | | | |
| 0.00011 | ref.negative.21:45658474, | | | | | | | | | | |
| 0.0011 | ref.negative.5:11562221, | | | | | | | | | | |
| 0.00013 | ref.negative.14:98734567 | | | | | | | | | | |
| −5e−05 | additive.9:117568766, | 4 | 0.39215686 | 0.49019608 | 0.48692308 | 0.5 | 0.48387097 | 0.5 | 0.38461538 | 0.6 | colon.bma.model.cluster.Resi- |
| 0.00129 | alt.recessive.9:117568766, | | | | | | | | | | dent.macrophages.RDS |
| 0 | alt.negative.9:117568766, | | | | | | | | | | |
| 0.00129 | ref.recessive.21:45658474, | | | | | | | | | | |
| 0.00017 | ref.negative.21:45658474, | | | | | | | | | | |
| 0.00131 | ref.negative.5:11562221, | | | | | | | | | | |
| 0.00064 | alt.negative.16:11331509, | | | | | | | | | | |
| 0.00033 | ref.negative.16:11331509 | | | | | | | | | | |
| 0.00012 | additive.9:117568766, | 4 | 0.39215686 | 0.49019608 | 0.5 | 0.5 | 0.48387097 | 0.5 | 0.38461538 | 0.6 | colon.bma.model.cluster.Resi- |
| 0.00116 | alt.recessive.9:117568766, | | | | | | | | | | dent.macrophages.RDS |
| 0 | alt.negative.9:117568766, | | | | | | | | | | |
| 0.00146 | ref.recessive.21:45658474, | | | | | | | | | | |
| 0.00013 | ref.negative.21:45658474, | | | | | | | | | | |
| 0.0011 | ref.negative.5:11562221, | | | | | | | | | | |
| 0.00104 | ref.negative.16:6147645 | | | | | | | | | | |
| 0.00038 | additive.9:117568766, | 4 | 0.47058824 | 0.49019608 | 0.54076923 | 0.5 | 0.48148148 | 0.5 | 0.46153846 | 0.52 | colon.bma.model.cluster.Resi- |
| 0.00114 | alt.recessive.9:117568766, | | | | | | | | | | dent.macrophages.RDS |
| 0 | alt.negative.9:117568766, | | | | | | | | | | |
| 0.00095 | ref.negative.5:11562221, | | | | | | | | | | |
| 0.00173 | ref.negative.20:62305274, | | | | | | | | | | |
| 0.00041 | ref.negative.14:98734567 | | | | | | | | | | |
| 0.00063 | additive.9:117568766, | 4 | 0.50980392 | 0.49019608 | 0.50615385 | 0.5 | 0.48 | 0.5 | 0.5 | 0.48 | colon.bma.model.cluster.Resi- |
| 0.00058 | alt.recessive.9:117568766, | | | | | | | | | | dent.macrophages.RDS |
| 0 | alt.negative.9:117568766, | | | | | | | | | | |
| 0.00105 | ref.negative.5:11562221, | | | | | | | | | | |
| 0.00125 | ref.negative.20:62305274, | | | | | | | | | | |
| 0.00069 | ref.negative.16:6147645 | | | | | | | | | | |
| 0.00065 | additive.9:117568766, | 4 | 0.50980392 | 0.49019608 | 0.48846154 | 0.5 | 0.48 | 0.5 | 0.5 | 0.48 | colon.bma.model.cluster.Resi- |
| 0.00068 | alt.recessive.9:117568766, | | | | | | | | | | dent.macrophages.RDS |
| 0 | alt.negative.9:117568766, | | | | | | | | | | |
| 0.00106 | ref.negative.5:11562221, | | | | | | | | | | |
| 0.00082 | ref.negative.14:98734567, | | | | | | | | | | |
| 0.00083 | ref.negative.16:6147645 | | | | | | | | | | |
| 0.00153 | ref.recessive.21:45658474, | 4 | 0.35294118 | 0.49019608 | 0.50076923 | 0.5 | 0.48484848 | 0.5 | 0.34615385 | 0.64 | colon.bma.model.cluster.Resi- |
| 0.00111 | ref.negative.21:45658474, | | | | | | | | | | dent.macrophages.RDS |
| 0.00104 | ref.negative.11:128443099, | | | | | | | | | | |
| 0.00049 | alt.negative.16:11331509, | | | | | | | | | | |
| 0.00038 | ref.negative.16:11331509, | | | | | | | | | | |
| 0.00099 | ref.negative.16:6147645 | | | | | | | | | | |
| 0.00149 | ref.negative.5:11562221, | 4 | 0.43137255 | 0.49019608 | 0.54461538 | 0.5 | 0.48275862 | 0.5 | 0.42307692 | 0.56 | colon.bma.model.cluster.Resi- |
| 0.00068 | ref.negative.11:128443099, | | | | | | | | | | dent.macrophages.RDS |
| 2e−04 | ref.negative.14:98734567, | | | | | | | | | | |
| 0.00065 | alt.negative.16:11331509, | | | | | | | | | | |
| 0.00025 | ref.negative.16:11331509 | | | | | | | | | | |
| 0.00122 | ref.negative.5:11562221, | 4 | 0.31372549 | 0.49019608 | 0.51076923 | 0.5 | 0.48571429 | 0.5 | 0.30769231 | 0.68 | colon.bma.model.cluster.Resi- |
| 0.001 | ref.negative.11:128443099, | | | | | | | | | | dent.macrophages.RDS |
| 0.00061 | ref.negative.14:98734567, | | | | | | | | | | |
| 0.00072 | ref.negative.16:6147645 | | | | | | | | | | |
| 0.00173 | ref.negative.5:11562221, | 4 | 0.31372549 | 0.49019608 | 0.46615385 | 0.5 | 0.48571429 | 0.5 | 0.30769231 | 0.68 | colon.bma.model.cluster.Resi- |
| 0.00047 | ref.negative.14:98734567, | | | | | | | | | | dent.macrophages.RDS |
| 0.00091 | alt.negative.16:11331509, | | | | | | | | | | |
| 0.00034 | ref.negative.16:11331509, | | | | | | | | | | |
| 0.00048 | ref.negative.16:6147645 | | | | | | | | | | |
| 0.00102 | ref.negative.20:62305274, | 4 | 0.43137255 | 0.49019608 | 0.51461538 | 0.5 | 0.48275862 | 0.5 | 0.42307692 | 0.56 | colon.bma.model.cluster.Resi- |
| 0.00061 | ref.negative.14:98734567, | | | | | | | | | | dent.macrophages.RDS |
| 0.00044 | alt.negative.16:11331509, | | | | | | | | | | |
| 0.00025 | ref.negative.16:11331509, | | | | | | | | | | |
| 0.00057 | ref.negative.16:6147645 | | | | | | | | | | |
| 0.00081 | additive.9:117568766, | 4 | 0.49019608 | 0.47058824 | 0.48923077 | 0.48 | 0.46153846 | 0.48 | 0.46153846 | 0.48 | colon.bma.model.cluster.Resi- |
| 0.00066 | alt.recessive.9:117568766, | | | | | | | | | | dent.macrophages.RDS |
| 0 | alt.negative.9:117568766, | | | | | | | | | | |
| 0.00148 | ref.negative.20:62305274, | | | | | | | | | | |
| 0.00081 | ref.negative.14:98734567, | | | | | | | | | | |
| 0.00091 | ref.negative.16:6147645 | | | | | | | | | | |
| 0.00059 | ref.negative.20:62305274, | 4 | 0.49019608 | 0.47058824 | 0.48153846 | 0.48 | 0.46153846 | 0.48 | 0.46153846 | 0.48 | colon.bma.model.cluster.Resi- |
| 0.00072 | ref.negative.11:128443099, | | | | | | | | | | dent.macrophages.RDS |
| 0.00045 | alt.negative.16:11331509, | | | | | | | | | | |
| 0.00031 | ref.negative.16:11331509, | | | | | | | | | | |
| 0.00045 | ref.negative.16:6147645 | | | | | | | | | | |
| 6e−04 | additive.9:117568766, | 4 | 0.45098039 | 0.47058824 | 0.50230769 | 0.47826087 | 0.46428571 | 0.47826087 | 0.42307692 | 0.52 | colon.bma.model.cluster.Resi- |
| 0.00059 | alt.recessive.9:117568766, | | | | | | | | | | dent.macrophages.RDS |
| 0 | alt.negative.9:117568766, | | | | | | | | | | |
| 0.00089 | ref.negative.5:11562221, | | | | | | | | | | |
| 0.00088 | ref.negative.11:128443099, | | | | | | | | | | |
| 0.00065 | alt.negative.16:11331509, | | | | | | | | | | |
| −4e−05 | ref.negative.16:11331509 | | | | | | | | | | |
| 0.00058 | additive.9:117568766, | 4 | 0.45098039 | 0.47058824 | 0.49384615 | 0.47826087 | 0.46428571 | 0.47826087 | 0.42307692 | 0.52 | colon.bma.model.cluster.Resi- |
| 0.00093 | alt.recessive.9:117568766, | | | | | | | | | | dent.macrophages.RDS |
| 0 | alt.negative.9:117568766, | | | | | | | | | | |
| 0.00072 | ref.negative.5:11562221, | | | | | | | | | | |
| 0.00099 | ref.negative.11:128443099, | | | | | | | | | | |
| 0.00091 | ref.negative.16:6147645 | | | | | | | | | | |
| 0.00086 | additive.9:117568766, | 4 | 0.45098039 | 0.47058824 | 0.48923077 | 0.47826087 | 0.46428571 | 0.47826087 | 0.42307692 | 0.52 | colon.bma.model.cluster.Resi- |
| 0.00056 | alt.recessive.9:117568766, | | | | | | | | | | dent.macrophages.RDS |
| 0 | alt.negative.9:117568766, | | | | | | | | | | |
| 0.00083 | ref.negative.20:62305274, | | | | | | | | | | |
| 0.00105 | ref.negative.11:128443099, | | | | | | | | | | |
| 0.00103 | ref.negative.16:6147645 | | | | | | | | | | |
| 0.00057 | additive.9:117568766, | 4 | 0.45098039 | 0.47058824 | 0.49846154 | 0.47826087 | 0.46428571 | 0.4826087 | 0.42307692 | 0.52 | colon.bma.model.cluster.Resi- |
| 0.00081 | alt.recessive.9:117568766, | | | | | | | | | | dent.macrophages.RDS |
| 0 | alt.negative.9:117568766, | | | | | | | | | | |
| 0.00092 | ref.negative.11:128443099, | | | | | | | | | | |
| 0.00052 | refnegative.14:98734567, | | | | | | | | | | |
| 0.00052 | alt.negative.16:11331509, | | | | | | | | | | |
| −1e−04 | ref.negative.16:11331509 | | | | | | | | | | |
| 0.00022 | additive.9:117568766, | 4 | 0.41176471 | 0.47058824 | 0.47615385 | 0.47619048 | 0.46666667 | 0.4619048 | 0.38461538 | 0.56 | colon.bma.model.cluster.Resi- |
| 0.001 | alt.recessive.9:117568766, | | | | | | | | | | dent.macrophages.RDS |
| 0 | alt.negative.9:117568766, | | | | | | | | | | |
| 0.00128 | ref.recessive.21:45658474, | | | | | | | | | | |
| 0.00029 | ref.negative.21:45658474, | | | | | | | | | | |
| 0.00041 | ref.negative.14:98734567, | | | | | | | | | | |
| 0.00058 | alt.negative.16:11331509, | | | | | | | | | | |
| 8e−05 | ref.negative.16:11331509 | | | | | | | | | | |
| 0.00155 | ref.negative.5:11562221, | 4 | 0.41176471 | 0.47058824 | 0.52 | 0.47619048 | 0.46666667 | 0.47619048 | 0.38461538 | 0.56 | colon.bma.model.cluster.Resi- |
| 0.00073 | ref.negative.20:62305274, | | | | | | | | | | dent.macrophages.RDS |
| 0.00056 | ref.negative.11:128443099, | | | | | | | | | | |
| 0.00081 | alt.negative.16:11331509, | | | | | | | | | | |
| 0.00037 | ref.negative.16:11331509 | | | | | | | | | | |
| 0.00133 | ref.negative.5:11562221, | 4 | 0.33333333 | 0.47058824 | 0.50076923 | 0.47058824 | 0.47058824 | 0.47058824 | 0.30769231 | 0.64 | colon.bma.model.cluster.Resi- |
| 0.00072 | ref.negative.11:128443099, | | | | | | | | | | dent.macrophages.RDS |
| 0.00051 | alt.negative.16:11331509, | | | | | | | | | | |
| 0.00025 | ref.negative.16:11331509, | | | | | | | | | | |
| 0.00056 | ref.negative.16:6147645 | | | | | | | | | | |
| 0.00081 | ref.negative.11:128443099, | 4 | 0.33333333 | 0.47058824 | 0.46615385 | 0.47058824 | 0.47058824 | 0.47058824 | 0.30769231 | 0.64 | colon.bma.model.cluster.Resi- |
| 0.00033 | ref.negative.14:98734567, | | | | | | | | | | dent.macrophages.RDS |
| 0.00044 | alt.negative.16:11331509, | | | | | | | | | | |
| 0.00011 | ref.negative.16:11331509, | | | | | | | | | | |
| 0.00052 | ref.negative.16:6147645 | | | | | | | | | | |
| 0.00051 | additive.9:117568766, | 4 | 0.43137255 | 0.45098039 | 0.52538462 | 0.45454545 | 0.44827586 | 0.45454545 | 0.38461538 | 0.52 | colon.bma.model.cluster.Resi- |
| 0.00087 | alt.recessive.9:117568766, | | | | | | | | | | dent.macrophages.RDS |
| 0 | alt.negative.9:117568766, | | | | | | | | | | |
| 0.00075 | ref.negative.5:11562221, | | | | | | | | | | |
| 0.00109 | ref.negative.20:62305274, | | | | | | | | | | |
| 0.00082 | ref.negative.11:128443099 | | | | | | | | | | |
| 5e−04 | additive.9:117568766, | 4 | 0.43137255 | 0.45098039 | 0.53307692 | 0.45454545 | 0.44827586 | 0.45454545 | 0.38461538 | 0.52 | colon.bma.model.cluster.Resi- |
| 9e−04 | alt.recessive.9:117568766, | | | | | | | | | | dent.macrophages.RDS |
| 0 | alt.negative.9:117568766, | | | | | | | | | | |
| 0.00085 | ref.negative.5:11562221, | | | | | | | | | | |
| 0.00099 | ref.negative.11:128443099, | | | | | | | | | | |
| 0.00054 | ref.negative.14:98734567 | | | | | | | | | | |
| 0.00069 | additive.9:117568766, | 4 | 0.43137255 | 0.45098039 | 0.49076923 | 0.45454545 | 0.44827586 | 0.45454545 | 0.38461538 | 0.52 | colon.bma.model.cluster.Resi- |
| 6e−04 | alt.recessive.9:117568766, | | | | | | | | | | dent.macrophages.RDS |
| 0 | alt.negative.9:117568766, | | | | | | | | | | |
| 0.00101 | ref.negative.20:62305274, | | | | | | | | | | |
| 0.00084 | ref.negative.11:128443099, | | | | | | | | | | |
| 0.00041 | alt.negative.16:11331509, | | | | | | | | | | |
| −0.00022 | ref.negative.16:11331509 | | | | | | | | | | |
| 0.00076 | additive.9:117568766, | 4 | 0.43137255 | 0.45098039 | 0.50076923 | 0.45454545 | 0.44827586 | 0.45454545 | 0.38461538 | 0.52 | colon.bma.model.cluster.Resi- |
| 0.00053 | alt.recessive.9:117568766, | | | | | | | | | | dent.macrophages.RDS |
| 0 | alt.negative.9:117568766, | | | | | | | | | | |
| 0.00098 | ref.negative.11:128443099, | | | | | | | | | | |
| 0.00103 | refnegative.14:98734567, | | | | | | | | | | |
| 0.00075 | ref.negative.16:6147645 | | | | | | | | | | |
| 2e−04 | additive.9:117568766, | 4 | 0.39215686 | 0.45098039 | 0.47846154 | 0.45 | 0.4516129 | 0.45 | 0.34615385 | 0.56 | colon.bma.model.cluster.Resi- |
| 0.00102 | alt.recessive.9:117568766, | | | | | | | | | | dent.macrophages.RDS |
| 0 | alt.negative.9:117568766, | | | | | | | | | | |
| 0.00145 | ref.recessive.21:45658474, | | | | | | | | | | |
| 2e−04 | ref.negative.21:45658474, | | | | | | | | | | |
| 0.00055 | ref.negative.14:98734567, | | | | | | | | | | |
| 0.00102 | ref.negative.16:6147645 | | | | | | | | | | |
| 0.00029 | additive.9:117568766, | 4 | 0.31372549 | 0.45098039 | 0.48692308 | 0.4375 | 0.45714286 | 0.4375 | 0.26923077 | 0.64 | colon.bma.model.cluster.Resi- |
| 0.00094 | alt.recessive.9:117568766, | | | | | | | | | | dent.macrophages.RDS |
| 0 | alt.negative.9:117568766, | | | | | | | | | | |
| 0.00128 | ref.recessive.21:45658474, | | | | | | | | | | |
| 0.00044 | ref.negative.21:45658474, | | | | | | | | | | |
| 0.00044 | alt.negative.16:11331509, | | | | | | | | | | |
| 4e−05 | ref.negative.16:11331509, | | | | | | | | | | |
| 0.00086 | ref.negative.16:6147645 | | | | | | | | | | |
| 0.00062 | additive.9:117568766, | 4 | 0.31372549 | 0.45098039 | 0.51923077 | 0.4375 | 0.45714286 | 0.4375 | 0.26923077 | 0.64 | colon.bma.model.cluster.Resi- |
| 0.00084 | alt.recessive.9:117568766, | | | | | | | | | | dent.macrophages.RDS |
| 0 | alt.negative.9:117568766, | | | | | | | | | | |
| 0.00128 | ref.negative.5:11562221, | | | | | | | | | | |
| 0.00153 | ref.negative.20:62305274, | | | | | | | | | | |
| 0.00068 | alt.negative.16:11331509, | | | | | | | | | | |
| 4e−05 | ref.negative.16:11331509 | | | | | | | | | | |
| 0.00064 | additive.9:117568766, | 4 | 0.31372549 | 0.45098039 | 0.51615385 | 0.4375 | 0.45714286 | 0.4375 | 0.26923077 | 0.64 | colon.bma.model.cluster.Resi- |
| 0.00074 | alt.recessive.9:117568766, | | | | | | | | | | dent.macrophages.RDS |
| 0 | alt.negative.9:117568766, | | | | | | | | | | |
| 0.00118 | ref.negative.5:11562221, | | | | | | | | | | |
| 7e−04 | ref.negative.14:98734567, | | | | | | | | | | |
| 0.00072 | alt.negative.16:11331509, | | | | | | | | | | |
| 0.00015 | ref.negative.16:11331509 | | | | | | | | | | |
| 0.00066 | additive.9:117568766, | 4 | 0.31372549 | 0.45098039 | 0.51076923 | 0.4375 | 0.45714286 | 0.4375 | 0.26923077 | 0.64 | colon.bma.model.cluster.Resi- |
| 0.00077 | alt.recessive.9:117568766, | | | | | | | | | | dent.macrophages.RDS |
| 0 | alt.negative.9:117568766, | | | | | | | | | | |
| 0.00156 | ref.negative.20:62305274, | | | | | | | | | | |
| 0.00053 | ref.negative.14:98734567, | | | | | | | | | | |
| 0.00071 | alt.negative.16:11331509, | | | | | | | | | | |
| 0.00013 | ref.negative.16:11331509 | | | | | | | | | | |
| 0.00067 | additive.9:117568766, | 4 | 0.31372549 | 0.45098039 | 0.46769231 | 0.4375 | 0.45714286 | 0.4375 | 0.26923077 | 0.64 | colon.bma.model.cluster.Resi- |
| 0.00078 | alt.recessive.9:117568766, | | | | | | | | | | dent.macrophages.RDS |
| 0 | alt.negative.9:117568766, | | | | | | | | | | |
| 0.00071 | ref.negative.14:98734567, | | | | | | | | | | |
| 0.00049 | alt.negative.16:11331509, | | | | | | | | | | |
| −0.00012 | ref.negative.16:11331509, | | | | | | | | | | |
| 0.00075 | ref.negative.16:6147645 | | | | | | | | | | |
| 0.00128 | ref.negative.5:11562221, | 4 | 0.31372549 | 0.45098039 | 0.51307692 | 0.4375 | 0.45714286 | 0.4375 | 0.26923077 | 0.64 | colon.bma.model.cluster.Resi- |
| 0.00055 | ref.negative.20:62305274, | | | | | | | | | | dent.macrophages.RDS |
| 0.00082 | ref.negative.11:128443099, | | | | | | | | | | |
| 0.00067 | ref.negative.16:6147645 | | | | | | | | | | |
| 0.00046 | ref.negative.20:62305274, | 4 | 0.31372549 | 0.45098039 | 0.49 | 0.4375 | 0.45714286 | 0.4375 | 0.26923077 | 0.64 | colon.bma.model.cluster.Resi- |
| 0.00075 | ref.negative.11:128443099, | | | | | | | | | | dent.macrophages.RDS |
| 0.00053 | ref.negative.14:98734567, | | | | | | | | | | |
| 0.00067 | ref.negative.16:6147645 | | | | | | | | | | |
| 0.00064 | additive.9:117568766, | 4 | 0.41176471 | 0.43137255 | 0.51692308 | 0.42857143 | 0.43333333 | 0.42857143 | 0.34615385 | 0.52 | colon.bma.model.cluster.Resi- |
| 0.00081 | alt.recessive.9:117568766, | | | | | | | | | | dent.macrophages.RDS |
| 0 | alt.negative.9:117568766, | | | | | | | | | | |
| 0.00109 | ref.negative.20:62305274, | | | | | | | | | | |
| 0.00083 | ref.negative.11:128443099, | | | | | | | | | | |
| 2e−04 | refnegative.14:98734567 | | | | | | | | | | |
| 0.00071 | ref.negative.20:62305274, | 4 | 0.41176471 | 0.43137255 | 0.49923077 | 0.42857143 | 0.43333333 | 0.42857143 | 0.34615385 | 0.52 | colon.bma.model.cluster.Resi- |
| 0.00067 | ref.negative.11:128443099, | | | | | | | | | | dent.macrophages.RDS |
| 0.00042 | ref.negative.14:98734567, | | | | | | | | | | |
| 0.00076 | alt.negative.16:11331509, | | | | | | | | | | |
| 0.00036 | ref.negative.16:11331509 | | | | | | | | | | |
| 0.00075 | additive.9:117568766, | 4 | 0.37254902 | 0.43137255 | 0.47769231 | 0.42105263 | 0.4375 | 0.42105263 | 0.30769231 | 0.56 | colon.bma.model.cluster.Resi- |
| 0.00072 | alt.recessive.9:117568766, | | | | | | | | | | dent.macrophages.RDS |
| 0 | alt.negative.9:117568766, | | | | | | | | | | |
| 0.001 | ref.negative.11:128443099, | | | | | | | | | | |
| 0.00042 | alt.negative.16:11331509, | | | | | | | | | | |
| −0.00014 | ref.negative.16:11331509, | | | | | | | | | | |
| 0.00077 | ref.negative.16:6147645 | | | | | | | | | | |
| 0.00074 | additive.9:117568766, | 4 | 0.33333333 | 0.43137255 | 0.46 | 0.41176471 | 0.44117647 | 0.41176471 | 0.26923077 | 0.6 | colon.bma.model.cluster.Resi- |
| 0.00047 | alt.recessive.9:117568766, | | | | | | | | | | dent.macrophages.RDS |
| 0 | alt.negative.9:117568766, | | | | | | | | | | |
| 0.00091 | ref.negative.5:11562221, | | | | | | | | | | |
| 0.00061 | alt.negative.16:11331509, | | | | | | | | | | |
| −6e−05 | ref.negative.16:11331509, | | | | | | | | | | |
| 0.00074 | ref.negative.16:6147645 | | | | | | | | | | |
| 0.00076 | additive.9:117568766, | 4 | 0.31372549 | 0.41176471 | 0.47538462 | 0.375 | 0.42857143 | 0.375 | 0.23076923 | 0.6 | colon.bma.model.cluster.Resi- |
| 0.00058 | alt.recessive.9:117568766, | | | | | | | | | | dent.macrophages.RDS |
| 0 | alt.negative.9:117568766, | | | | | | | | | | |
| 0.00126 | ref.negative.20:62305274, | | | | | | | | | | |
| 0.00048 | alt.negative.16:11331509, | | | | | | | | | | |
| −0.00016 | ref.negative.16:11331509, | | | | | | | | | | |
| 0.00061 | ref.negative.16:6147645 | | | | | | | | | | |
| 0.0016 | ref.recessive.21:45658474, | 4 | 0.31372549 | 0.41176471 | 0.47307692 | 0.375 | 0.42857143 | 0.375 | 0.23076923 | 0.6 | colon.bma.model.cluster.Resi- |
| 0.00075 | ref.negative.21:45658474, | | | | | | | | | | dent.macrophages.RDS |
| 0.00112 | ref.negative.5:11562221, | | | | | | | | | | |
| 0.00103 | ref.negative.11:128443099, | | | | | | | | | | |
| 0.00108 | ref.negative.16:6147645 | | | | | | | | | | |
| 0.00158 | ref.recessive.21:45658474, | 4 | 0.31372549 | 0.41176471 | 0.47384615 | 0.375 | 0.42857143 | 0.375 | 0.23076923 | 0.6 | colon.bma.model.cluster.Resi- |
| 0.00055 | ref.negative.21:45658474, | | | | | | | | | | dent.macrophages.RDS |
| 0.00134 | ref.negative.5:11562221, | | | | | | | | | | |
| 5e−04 | ref.negative.14:98734567, | | | | | | | | | | |
| 0.00079 | ref.negative.16:6147645 | | | | | | | | | | |
| 0.00142 | ref.recessive.21:45658474, | 4 | 0.31372549 | 0.41176471 | 0.46923077 | 0.375 | 0.42857143 | 0.375 | 0.23076923 | 0.6 | colon.bma.model.cluster.Resi- |
| 7e−04 | ref.negative.21:45658474, | | | | | | | | | | dent.macrophages.RDS |
| 0.00143 | ref.negative.5:11562221, | | | | | | | | | | |
| 0.00058 | alt.negative.16:11331509, | | | | | | | | | | |
| 0.00028 | ref.negative.16:11331509, | | | | | | | | | | |
| 0.00077 | ref.negative.16:6147645 | | | | | | | | | | |
| 0.0014 | ref.recessive.21:45658474, | 4 | 0.31372549 | 0.41176471 | 0.47615385 | 0.375 | 0.42857143 | 0.375 | 0.23076923 | 0.6 | colon.bma.model.cluster.Resi- |
| 0.00098 | ref.negative.21:45658474, | | | | | | | | | | dent.macrophages.RDS |
| 0.00073 | ref.negative.20:62305274, | | | | | | | | | | |
| 0.00059 | alt.negative.16:11331509, | | | | | | | | | | |
| 0.00035 | ref.negative.16:11331509, | | | | | | | | | | |
| 8e−04 | ref.negative.16:6147645 | | | | | | | | | | |
| 0.00154 | ref.recessive.21:45658474, | 4 | 0.31372549 | 0.41176471 | 0.48153846 | 0.375 | 0.42857143 | 0.375 | 0.23076923 | 0.6 | colon.bma.model.cluster.Resi- |
| 0.00094 | ref.negative.21:45658474, | | | | | | | | | | dent.macrophages.RDS |
| 0.00091 | ref.negative.11:128443099, | | | | | | | | | | |
| 0.00038 | ref.negative.14:98734567, | | | | | | | | | | |
| 0.00097 | ref.negative.16:6147645 | | | | | | | | | | |
| 0.00166 | ref.recessive.21:45658474, | 4 | 0.31372549 | 0.41176471 | 0.45923077 | 0.375 | 0.42857143 | 0.375 | 0.23076923 | 0.6 | colon.bma.model.cluster.Resi- |
| 0.00074 | ref.negative.21:45658474, | | | | | | | | | | dent.macrophages.RDS |
| 0.00059 | ref.negative.14:98734567, | | | | | | | | | | |
| 0.00061 | alt.negative.16:11331509, | | | | | | | | | | |
| 0.00019 | ref.negative.16:11331509, | | | | | | | | | | |
| 0.00081 | ref.negative.16:6147645 | | | | | | | | | | |
| 0.00133 | ref.recessive.21:45658474, | 3 | 0.31372549 | 0.56862745 | 0.51769231 | 0.625 | 0.54285714 | 0.625 | 0.38461538 | 0.76 | colon.bma.model.cluster.Resi- |
| 0.00062 | ref.negative.21:45658474, | | | | | | | | | | dent.macrophages.RDS |
| 0.0012 | ref.negative.5:11562221, | | | | | | | | | | |
| 0.00096 | ref.negative.11:128443099 | | | | | | | | | | |
| 3e−05 | additive.9:117568766, | 3 | 0.33333333 | 0.54901961 | 0.52538462 | 0.58823529 | 0.52941176 | 0.58823529 | 0.38461538 | 0.72 | colon.bma.model.cluster.Resi- |
| 0.00116 | alt.recessive.9:117568766, | | | | | | | | | | dent.macrophages.RDS |
| 0 | alt.negative.9:117568766, | | | | | | | | | | |
| 0.00139 | ref.recessive.21:45658474, | | | | | | | | | | |
| 0.00049 | ref.negative.21:45658474, | | | | | | | | | | |
| 0.00122 | ref.negative.11:128443099 | | | | | | | | | | |
| 0.00134 | ref.recessive.21:45658474, | 3 | 0.33333333 | 0.54901961 | 0.53615385 | 0.58823529 | 0.52941176 | 0.58823529 | 0.38461538 | 0.72 | colon.bma.model.cluster.Resi- |
| 0.00082 | ref.negative.21:45658474, | | | | | | | | | | dent.macrophages.RDS |
| 5e−04 | ref.negative.20:62305274, | | | | | | | | | | |
| 0.00088 | ref.negative.11:128443099 | | | | | | | | | | |
| 0.00143 | ref.recessive.21:45658474, | 3 | 0.33333333 | 0.54901961 | 0.50692308 | 0.58823529 | 0.52941176 | 0.58823529 | 0.38461538 | 0.72 | colon.bma.model.cluster.Resi- |
| 0.00091 | ref.negative.21:45658474, | | | | | | | | | | dent.macrophages.RDS |
| 0.00104 | ref.negative.11:128443099, | | | | | | | | | | |
| 0.00024 | alt.negative.16:11331509, | | | | | | | | | | |
| 0.00033 | ref.negative.16:11331509 | | | | | | | | | | |
| 0.00154 | ref.recessive.21:45658474, | 3 | 0.33333333 | 0.54901961 | 0.5 | 0.58823529 | 0.52941176 | 0.58823529 | 0.38461538 | 0.72 | colon.bma.model.cluster.Resi- |
| 0.00087 | ref.negative.21:45658474, | | | | | | | | | | dent.macrophages.RDS |
| 0.00096 | ref.negative.11:128443099, | | | | | | | | | | |
| 0.00091 | ref.negative.16:6147645 | | | | | | | | | | |
| −2e−05 | additive.9:117568766, | 3 | 0.41176471 | 0.54901961 | 0.56615385 | 0.57142857 | 0.53333333 | 0.57142857 | 0.46153846 | 0.64 | colon.bma.model.cluster.Resi- |
| 0.00131 | alt.recessive.9:117568766, | | | | | | | | | | dent.macrophages.RDS |
| 0 | alt.negative.9:117568766, | | | | | | | | | | |
| 0.00127 | ref.recessive.21:45658474, | | | | | | | | | | |
| 0.00034 | refnegative.21:45658474, | | | | | | | | | | |
| 0.00142 | ref.negative.20:62305274 | | | | | | | | | | |
| 0.00127 | ref.negative.5:11562221, | 3 | 0.82352941 | 0.60784314 | 0.60307692 | 0.57142857 | 0.77777778 | 0.57142857 | 0.92307692 | 0.28 | colon.bma.model.cluster.Resi- |
| 0.00107 | ref.negative.20:62305274, | | | | | | | | | | dent.macrophages.RDS |
| 0.00037 | ref.negative.14:98734567 | | | | | | | | | | |
| 0.0014 | ref.recessive.21:45658474, | 3 | 0.45098039 | 0.54901961 | 0.53076923 | 0.56521739 | 0.53571429 | 0.56521739 | 0.5 | 0.6 | colon.bma.model.cluster.Resi- |
| 0.00048 | ref.negative.21:45658474, | | | | | | | | | | dent.macrophages.RDS |
| 0.0013 | ref.negative.5:11562221, | | | | | | | | | | |
| 0.00099 | ref.negative.20:62305274 | | | | | | | | | | |
| 0.00135 | ref.recessive.21:45658474, | 3 | 0.31372549 | 0.52941176 | 0.52230769 | 0.5625 | 0.51428571 | 0.5625 | 0.34615385 | 0.72 | colon.bma.model.cluster.Resi- |
| 0.00075 | ref.negative.21:45658474, | | | | | | | | | | dent.macrophages.RDS |
| 0.00106 | ref.negative.20:62305274, | | | | | | | | | | |
| 0.00053 | alt.negative.16:11331509, | | | | | | | | | | |
| 4e−04 | ref.negative.16:11331509 | | | | | | | | | | |
| 0.00166 | ref.recessive.21:45658474, | 3 | 0.31372549 | 0.52941176 | 0.50846154 | 0.5625 | 0.51428571 | 0.5625 | 0.34615385 | 0.72 | colon.bma.model.cluster.Resi- |
| 0.00078 | ref.negative.21:45658474, | | | | | | | | | | dent.macrophages.RDS |
| 0.001 | ref.negative.11:128443099, | | | | | | | | | | |
| 0.00016 | ref.negative.14:98734567 | | | | | | | | | | |
| 0.00118 | ref.negative.5:11562221, | 3 | 0.60784314 | 0.54901961 | 0.56692308 | 0.5483871 | 0.55 | 0.5483871 | 0.65384615 | 0.44 | colon.bma.model.cluster.Resi- |
| 0.00074 | ref.negative.11:128443099, | | | | | | | | | | dent.macrophages.RDS |
| 0.00028 | ref.negative.14:98734567 | | | | | | | | | | |
| 0.00128 | ref.recessive.21:45658474, | 3 | 0.43137255 | 0.52941176 | 0.53153846 | 0.54545455 | 0.51724138 | 0.54545455 | 0.46153846 | 0.6 | colon.bma.model.cluster.Resi- |
| 0.00071 | ref.negative.21:45658474, | | | | | | | | | | dent.macrophages.RDS |
| 0.00104 | ref.negative.20:62305274, | | | | | | | | | | |
| 2e−04 | ref.negative.14:98734567 | | | | | | | | | | |
| 0.00175 | ref.negative.5:11562221, | 3 | 0.43137255 | 0.52941176 | 0.53769231 | 0.54545455 | 0.51724138 | 0.54545455 | 0.46153846 | 0.6 | colon.bma.model.cluster.Resi- |
| 0.00124 | ref.negative.20:62305274, | | | | | | | | | | dent.macrophages.RDS |
| 0.00085 | alt.negative.16:11331509, | | | | | | | | | | |
| 0.00061 | ref.negative.16:11331509 | | | | | | | | | | |
| 0.00139 | ref.recessive.21:45658474, | 3 | 0.47058824 | 0.52941176 | 0.48307692 | 0.54166667 | 0.51851852 | 0.54166667 | 0.5 | 0.56 | colon.bma.model.cluster.Resi- |
| 0.00078 | ref.negative.21:45658474, | | | | | | | | | | dent.macrophages.RDS |
| 0.00099 | ref.negative.20:62305274, | | | | | | | | | | |
| 0.00083 | ref.negative.16:6147645 | | | | | | | | | | |
| 0.00127 | ref.negative.5:11562221, | 3 | 0.62745098 | 0.52941176 | 0.55923077 | 0.53125 | 0.52631579 | 0.53125 | 0.65384615 | 0.4 | colon.bma.model.cluster.Resi- |
| 0.00066 | ref.negative.20:62305274, | | | | | | | | | | dent.macrophages.RDS |
| 9e−04 | ref.negative.11:128443099 | | | | | | | | | | |
| 0.00146 | ref.recessive.21:45658474, | 3 | 0.33333333 | 0.50980392 | 0.49307692 | 0.52941176 | 0.5 | 0.52941176 | 0.34615385 | 0.68 | colon.bma.model.cluster.Resi- |
| 0.00064 | ref.negative.21:45658474, | | | | | | | | | | dent.macrophages.RDS |
| 0.00156 | ref.negative.5:11562221, | | | | | | | | | | |
| 0.00074 | alt.negative.16:11331509, | | | | | | | | | | |
| 0.00056 | ref.negative.16:11331509 | | | | | | | | | | |
| 0.00102 | ref.negative.20:62305274, | 3 | 0.41176471 | 0.50980392 | 0.52615385 | 0.52380952 | 0.5 | 0.52380952 | 0.42307692 | 0.6 | colon.bma.model.cluster.Resi- |
| 0.00016 | ref.negative.14:98734567, | | | | | | | | | | dent.macrophages.RDS |
| 0.00071 | alt.negative.16:11331509, | | | | | | | | | | |
| 0.00023 | ref.negative.16:11331509 | | | | | | | | | | |
| 0.00044 | additive.9:117568766, | 3 | 0.49019608 | 0.50980392 | 0.53076923 | 0.52 | 0.5 | 0.52 | 0.5 | 0.52 | colon.bma.model.cluster.Resi- |
| 0.00104 | alt.recessive.9:117568766, | | | | | | | | | | dent.macrophages.RDS |
| 0 | alt.negative.9:117568766, | | | | | | | | | | |
| 0.00093 | ref.negative.5:11562221, | | | | | | | | | | |
| 0.00072 | ref.negative.14:98734567 | | | | | | | | | | |
| 0.00068 | additive.9:117568766, | 3 | 0.52941176 | 0.50980392 | 0.49 | 0.51851852 | 0.5 | 0.51851852 | 0.53846154 | 0.48 | colon.bma.model.cluster.Resi- |
| 5e−04 | alt.recessive.9:117568766, | | | | | | | | | | dent.macrophages.RDS |
| 0 | alt.negative.9:117568766, | | | | | | | | | | |
| 0.00093 | ref.negative.5:11562221, | | | | | | | | | | |
| 8e−04 | ref.negative.16:6147645 | | | | | | | | | | |
| 0.00065 | ref.negative.20:62305274, | 3 | 0.60784314 | 0.50980392 | 0.54538462 | 0.51612903 | 0.5 | 0.51612903 | 0.61538462 | 0.4 | colon.bma.model.cluster.Resi- |
| 0.00072 | ref.negative.11:128443099, | | | | | | | | | | dent.macrophages.RDS |
| 0.00026 | ref.negative.14:98734567 | | | | | | | | | | |
| −0.00016 | additive.9:117568766, | 3 | 0.50980392 | 0.49019608 | 0.50692308 | 0.5 | 0.48 | 0.5 | 0.5 | 0.48 | colon.bma.model.cluster.Resi- |
| 0.00129 | alt.recessive.9:117568766, | | | | | | | | | | dent.macrophages.RDS |
| 0 | alt.negative.9:117568766, | | | | | | | | | | |
| 0.00131 | ref.recessive.21:45658474, | | | | | | | | | | |
| 9e−05 | ref.negative.21:45658474, | | | | | | | | | | |
| 0.00112 | ref.negative.5:11562221 | | | | | | | | | | |
| 0.00041 | additive.9:117568766, | 3 | 0.50980392 | 0.49019608 | 0.52846154 | 0.5 | 0.48 | 0.5 | 0.5 | 0.48 | colon.bma.model.cluster.Resi- |
| 0.00102 | alt.recessive.9:117568766, | | | | | | | | | | dent.macrophages.RDS |
| 0 | alt.negative.9:117568766, | | | | | | | | | | |
| 0.00097 | ref.negative.5:11562221, | | | | | | | | | | |
| 0.00148 | ref.negative.20:62305274 | | | | | | | | | | |
| 0.00057 | additive.9:117568766, | 3 | 0.47058824 | 0.49019608 | 0.52846154 | 0.5 | 0.48148148 | 0.5 | 0.46153846 | 0.52 | colon.bma.model.cluster.Resi- |
| 0.00084 | alt.recessive.9:117568766, | | | | | | | | | | dent.macrophages.RDS |
| 0 | alt.negative.9:117568766, | | | | | | | | | | |
| 0.00162 | ref.negative.20:62305274, | | | | | | | | | | |
| 0.00057 | ref.negative.14:98734567 | | | | | | | | | | |
| 0.00076 | additive.9:117568766, | 3 | 0.50980392 | 0.49019608 | 0.48461538 | 0.5 | 0.48 | 0.5 | 0.5 | 0.48 | colon.bma.model.cluster.Resi- |
| 0.00047 | alt.recessive.9:117568766, | | | | | | | | | | dent.macrophages.RDS |
| 0 | alt.negative.9:117568766, | | | | | | | | | | |
| 0.00152 | ref.negative.20:62305274, | | | | | | | | | | |
| 0.00061 | ref.negative.16:6147645 | | | | | | | | | | |
| 0.00078 | additive.9:117568766, | 3 | 0.50980392 | 0.49019608 | 0.47 | 0.5 | 0.48 | 0.5 | 0.5 | 0.48 | colon.bma.model.cluster.Resi- |
| 0.00061 | alt.recessive.9:117568766, | | | | | | | | | | dent.macrophages.RDS |
| 0 | alt.negative.9:117568766, | | | | | | | | | | |
| 0.00075 | ref.negative.14:98734567, | | | | | | | | | | |
| 0.00089 | ref.negative.16:6147645 | | | | | | | | | | |
| 0.00151 | ref.recessive.21:45658474, | 3 | 0.50980392 | 0.49019608 | 0.50307692 | 0.5 | 0.48 | 0.5 | 0.5 | 0.48 | colon.bma.model.cluster.Resi- |
| 0.00043 | ref.negative.21:45658474, | | | | | | | | | | dent.macrophages.RDS |
| 0.00135 | ref.negative.5:11562221, | | | | | | | | | | |
| −0.00011 | ref.negative.14:98734567 | | | | | | | | | | |
| 0.00147 | ref.recessive.21:45658474, | 3 | 0.31372549 | 0.49019608 | 0.51846154 | 0.5 | 0.48571429 | 0.5 | 0.30769231 | 0.68 | colon.bma.model.cluster.Resi- |
| 0.00071 | ref.negative.21:45658474, | | | | | | | | | | dent.macrophages.RDS |
| 0.00013 | ref.negative.14:98734567, | | | | | | | | | | |
| 0.00043 | alt.negative.16:11331509, | | | | | | | | | | |
| 0.00046 | ref.negative.16:11331509 | | | | | | | | | | |
| 0.00161 | ref.negative.5:11562221, | 3 | 0.43137255 | 0.49019608 | 0.53230769 | 0.5 | 0.48275862 | 0.5 | 0.42307692 | 0.56 | colon.bma.model.cluster.Resi- |
| 0.00067 | ref.negative.11:128443099, | | | | | | | | | | dent.macrophages.RDS |
| 0.00066 | alt.negative.16:11331509, | | | | | | | | | | |
| 4e−04 | ref.negative.16:11331509 | | | | | | | | | | |
| 0.00172 | ref.negative.5:11562221, | 3 | 0.43137255 | 0.49019608 | 0.50307692 | 0.5 | 0.48275862 | 0.5 | 0.42307692 | 0.56 | colon.bma.model.cluster.Resi- |
| 0.00034 | ref.negative.14:98734567, | | | | | | | | | | dent.macrophages.RDS |
| 0.00095 | alt.negative.16:11331509, | | | | | | | | | | |
| 0.00053 | ref.negative.16:11331509 | | | | | | | | | | |
| 0.00145 | ref.negative.5:11562221, | 3 | 0.47058824 | 0.49019608 | 0.48307692 | 0.5 | 0.48148148 | 0.5 | 0.46153846 | 0.52 | colon.bma.model.cluster.Resi- |
| 0.00057 | ref.negative.14:98734567, | | | | | | | | | | dent.macrophages.RDS |
| 8e−04 | ref.negative.16:6147645 | | | | | | | | | | |
| 0.0016 | ref.negative.5:11562221, | 3 | 0.31372549 | 0.49019608 | 0.46153846 | 0.5 | 0.48571429 | 0.5 | 0.30769231 | 0.68 | colon.bma.model.cluster.Resi- |
| 0.00072 | alt.negative.16:11331509, | | | | | | | | | | dent.macrophages.RDS |
| 5e−05 | ref.negative.16:11331509, | | | | | | | | | | |
| 0.00045 | ref.negative.16:6147645 | | | | | | | | | | |
| 9e−04 | ref.negative.20:62305274, | 3 | 0.31372549 | 0.49019608 | 0.50153846 | 0.5 | 0.48571429 | 0.5 | 0.30769231 | 0.68 | colon.bma.model.cluster.Resi- |
| 0.00059 | ref.negative.11:128443099, | | | | | | | | | | dent.macrophages.RDS |
| 0.00056 | alt.negative.16:11331509, | | | | | | | | | | |
| 0.00047 | ref.negative.16:11331509 | | | | | | | | | | |
| 0.00096 | ref.negative.20:62305274, | 3 | 0.43137255 | 0.49019608 | 0.49692308 | 0.5 | 0.48275862 | 0.5 | 0.42307692 | 0.56 | colon.bma.model.cluster.Resi- |
| 6e−04 | ref.negative.14:98734567, | | | | | | | | | | dent.macrophages.RDS |
| 0.00067 | ref.negative.16:6147645 | | | | | | | | | | |
| 9e−04 | ref.negative.11:128443099, | 3 | 0.31372549 | 0.49019608 | 0.53307692 | 0.5 | 0.48571429 | 0.5 | 0.30769231 | 0.68 | colon.bma.model.cluster.Resi- |
| 0.00024 | ref.negative.14:98734567, | | | | | | | | | | dent.macrophages.RDS |
| 0.00033 | alt.negative.16:11331509, | | | | | | | | | | |
| 0.00038 | ref.negative.16:11331509 | | | | | | | | | | |
| −5e−05 | additive.9:117568766, | 3 | 0.52941176 | 0.47058824 | 0.49923077 | 0.48148148 | 0.45833333 | 0.48148148 | 0.5 | 0.44 | colon.bma.model.cluster.Resi- |
| 0.00133 | alt.recessive.9:117568766, | | | | | | | | | | dent.macrophages.RDS |
| 0 | alt.negative.9:117568766, | | | | | | | | | | |
| 0.00131 | ref.recessive.21:45658474, | | | | | | | | | | |
| 0.00028 | refnegative.21:45658474, | | | | | | | | | | |
| 0.00029 | ref.negative.14:98734567 | | | | | | | | | | |
| 0.00049 | additive.9:117568766, | 3 | 0.45098039 | 0.47058824 | 0.52461538 | 0.47826087 | 0.46428571 | 0.47826087 | 0.42307692 | 0.52 | colon.bma.model.cluster.Resi- |
| 0.00076 | alt.recessive.9:117568766, | | | | | | | | | | dent.macrophages.RDS |
| 0 | alt.negative.9:117568766, | | | | | | | | | | |
| 0.00083 | ref.negative.5:11562221, | | | | | | | | | | |
| 0.00091 | ref.negative.11:128443099 | | | | | | | | | | |
| 0.00073 | additive.9:117568766, | 3 | 0.45098039 | 0.47058824 | 0.49384615 | 0.47826087 | 0.46428571 | 0.47826087 | 0.42307692 | 0.52 | colon.bma.model.cluster.Resi- |
| 0.00059 | alt.recessive.9:117568766, | | | | | | | | | | dent.macrophages.RDS |
| 0 | alt.negative.9:117568766, | | | | | | | | | | |
| 9e−04 | ref.negative.11:128443099, | | | | | | | | | | |
| 0.00054 | alt.negative.16:11331509, | | | | | | | | | | |
| −6e−05 | ref.negative.16:11331509 | | | | | | | | | | |
| 0.00076 | additive.9:117568766, | 3 | 0.45098039 | 0.47058824 | 0.48769231 | 0.47826087 | 0.46428571 | 0.47826087 | 0.42307692 | 0.52 | colon.bma.model.cluster.Resi- |
| 0.00061 | alt.recessive.9:117568766, | | | | | | | | | | dent.macrophages.RDS |
| 0 | alt.negative.9:117568766, | | | | | | | | | | |
| 0.00103 | ref.negative.11:128443099, | | | | | | | | | | |
| 0.00072 | ref.negative.16:6147645 | | | | | | | | | | |
| 0.00134 | ref.negative.5:11562221, | 3 | 0.45098039 | 0.47058824 | 0.50923077 | 0.47826087 | 0.46428571 | 0.47826087 | 0.42307692 | 0.52 | colon.bma.model.cluster.Resi- |
| 0.00118 | ref.negative.20:62305274, | | | | | | | | | | dent.macrophages.RDS |
| 0.00059 | ref.negative.16:6147645 | | | | | | | | | | |
| 0.00016 | additive.9:117568766, | 3 | 0.41176471 | 0.47058824 | 0.48076923 | 0.47619048 | 0.46666667 | 0.47619048 | 0.38461538 | 0.56 | colon.bma.model.cluster.Resi- |
| 0.00096 | alt.recessive.9:117568766, | | | | | | | | | | dent.macrophages.RDS |
| 0 | alt.negative.9:117568766, | | | | | | | | | | |
| 0.00125 | ref.recessive.21:45658474, | | | | | | | | | | |
| 0.00039 | refnegative.21:45658474, | | | | | | | | | | |
| 0.00056 | alt.negative.16:11331509, | | | | | | | | | | |
| 0.00035 | ref.negative.16:11331509 | | | | | | | | | | |
| 3e−04 | additive.9:117568766, | 3 | 0.41176471 | 0.47058824 | 0.48153846 | 0.47619048 | 0.46666667 | 0.47619048 | 0.38461538 | 0.56 | colon.bma.model.cluster.Resi- |
| 0.00093 | alt.recessive.9:117568766, | | | | | | | | | | dent.macrophages.RDS |
| 0 | alt.negative.9:117568766, | | | | | | | | | | |
| 0.00146 | ref.recessive.21:45658474, | | | | | | | | | | |
| 0.00038 | refnegative.21:45658474, | | | | | | | | | | |
| 0.00103 | ref.negative.16:6147645 | | | | | | | | | | |
| 0.00062 | additive.9:117568766, | 3 | 0.33333333 | 0.47058824 | 0.50076923 | 0.47058824 | 0.47058824 | 0.47058824 | 0.30769231 | 0.64 | colon.bma.model.cluster.Resi- |
| 0.00059 | alt.recessive.9:117568766, | | | | | | | | | | dent.macrophages.RDS |
| 0 | alt.negative.9:117568766, | | | | | | | | | | |
| 0.00121 | ref.negative.5:11562221, | | | | | | | | | | |
| 0.00076 | alt.negative.16:11331509, | | | | | | | | | | |
| 5e−05 | ref.negative.16:11331509 | | | | | | | | | | |
| 0.00128 | ref.negative.5:11562221, | 3 | 0.33333333 | 0.47058824 | 0.51076923 | 0.47058824 | 0.47058824 | 0.47058824 | 0.30769231 | 0.64 | colon.bma.model.cluster.Resi- |
| 0.00085 | ref.negative.11:128443099, | | | | | | | | | | dent.macrophages.RDS |
| 0.00071 | ref.negative.16:6147645 | | | | | | | | | | |
| 0.00095 | ref.negative.11:128443099, | 3 | 0.33333333 | 0.47058824 | 0.48461538 | 0.47058824 | 0.47058824 | 0.47058824 | 0.30769231 | 0.64 | colon.bma.model.cluster.Resi- |
| 0.00065 | ref.negative.14:98734567, | | | | | | | | | | dent.macrophages.RDS |
| 0.00081 | ref.negative.16:6147645 | | | | | | | | | | |
| 0.00061 | ref.negative.14:98734567, | 3 | 0.33333333 | 0.47058824 | 0.43692308 | 0.47058824 | 0.47058824 | 0.47058824 | 0.30769231 | 0.64 | colon.bma.model.cluster.Resi- |
| 7e−04 | alt.negative.16:11331509, | | | | | | | | | | dent.macrophages.RDS |
| 0.00036 | ref.negative.16:11331509, | | | | | | | | | | |
| 0.00062 | ref.negative.16:6147645 | | | | | | | | | | |
| 0.00109 | ref.negative.20:62305274, | 3 | 0.47058824 | 0.45098039 | 0.45230769 | 0.45833333 | 0.44444444 | 0.45833333 | 0.42307692 | 0.48 | colon.bma.model.cluster.Resi- |
| 0.00056 | alt.negative.16:11331509, | | | | | | | | | | dent.macrophages.RDS |
| 0.00025 | ref.negative.16:11331509, | | | | | | | | | | |
| 0.00058 | ref.negative.16:6147645 | | | | | | | | | | |
| 0.00073 | additive.9:117568766, | 3 | 0.43137255 | 0.45098039 | 0.51923077 | 0.45454545 | 0.44827586 | 0.45454545 | 0.38461538 | 0.52 | colon.bma.model.cluster.Resi- |
| 0.00063 | alt.recessive.9:117568766, | | | | | | | | | | dent.macrophages.RDS |
| 0 | alt.negative.9:117568766, | | | | | | | | | | |
| 0.00104 | ref.negative.20:62305274, | | | | | | | | | | |
| 0.00095 | ref.negative.11:128443099 | | | | | | | | | | |
| 0.00056 | additive.9:117568766, | 3 | 0.43137255 | 0.45098039 | 0.52538462 | 0.45454545 | 0.44827586 | 0.45454545 | 0.38461538 | 0.52 | colon.bma.model.cluster.Resi- |
| 7e−04 | alt.recessive.9:117568766, | | | | | | | | | | dent.macrophages.RDS |
| 0 | alt.negative.9:117568766, | | | | | | | | | | |
| 0.00091 | ref.negative.11:128443099, | | | | | | | | | | |
| 0.00043 | ref.negative.14:98734567 | | | | | | | | | | |
| 0.00075 | ref.negative.11:128443099, | 3 | 0.35294118 | 0.45098039 | 0.46153846 | 0.44444444 | 0.45454545 | 0.44444444 | 0.30769231 | 0.6 | colon.bma.model.cluster.Resi- |
| 0.00034 | alt.negative.16:11331509, | | | | | | | | | | dent.macrophages.RDS |
| 0.00015 | ref.negative.16:11331509, | | | | | | | | | | |
| 0.00052 | ref.negative.16:6147645 | | | | | | | | | | |
| 0.00073 | additive.9:117568766, | 3 | 0.31372549 | 0.45098039 | 0.49230769 | 0.4375 | 0.45714286 | 0.4375 | 0.26923077 | 0.64 | colon.bma.model.cluster.Resi- |
| 0.00038 | alt.recessive.9:117568766, | | | | | | | | | | dent.macrophages.RDS |
| 0 | alt.negative.9:117568766, | | | | | | | | | | |
| 0.00129 | ref.negative.20:62305274, | | | | | | | | | | |
| 0.00059 | alt.negative.16:11331509, | | | | | | | | | | |
| −3e−04 | ref.negative.16:11331509 | | | | | | | | | | |
| 0.00076 | additive.9:117568766, | 3 | 0.31372549 | 0.45098039 | 0.49538462 | 0.4375 | 0.45714286 | 0.4375 | 0.26923077 | 0.64 | colon.bma.model.cluster.Resi- |
| 0.00041 | alt.recessive.9:117568766, | | | | | | | | | | dent.macrophages.RDS |
| 0 | alt.negative.9:117568766, | | | | | | | | | | |
| 0.00053 | ref.negative.14:98734567, | | | | | | | | | | |
| 0.00075 | alt.negative.16:11331509, | | | | | | | | | | |
| −0.00019 | ref.negative.16:11331509 | | | | | | | | | | |
| 0.00092 | additive.9:117568766, | 3 | 0.33333333 | 0.43137255 | 0.45076923 | 0.41176471 | 0.44117647 | 0.41176471 | 0.26923077 | 0.6 | colon.bma.model.cluster.Resi- |
| 0.00026 | alt.recessive.9:117568766, | | | | | | | | | | dent.macrophages.RDS |
| 0 | alt.negative.9:117568766, | | | | | | | | | | |
| 0.00057 | alt.negative.16:11331509, | | | | | | | | | | |
| −0.00032 | ref.negative.16:11331509, | | | | | | | | | | |
| 6e−04 | ref.negative.16:6147645 | | | | | | | | | | |
| 0.00051 | ref.negative.20:62305274, | 3 | 0.33333333 | 0.43137255 | 0.48461538 | 0.41176471 | 0.44117647 | 0.41176471 | 0.26923077 | 0.6 | colon.bma.model.cluster.Resi- |
| 0.00089 | ref.negative.11:128443099, | | | | | | | | | | dent.macrophages.RDS |
| 0.00073 | ref.negative.16:6147645 | | | | | | | | | | |
| 0.00154 | ref.recessive.21:45658474, | 3 | 0.31372549 | 0.41176471 | 0.46538462 | 0.375 | 0.42857143 | 0.375 | 0.23076923 | 0.6 | colon.bma.model.cluster.Resi- |
| 0.00046 | ref.negative.21:45658474, | | | | | | | | | | dent.macrophages.RDS |
| 0.00146 | ref.negative.5:11562221, | | | | | | | | | | |
| 0.00081 | ref.negative.16:6147645 | | | | | | | | | | |
| 0.00133 | ref.recessive.21:45658474, | 3 | 0.31372549 | 0.41176471 | 0.47384615 | 0.375 | 0.42857143 | 0.375 | 0.23076923 | 0.6 | colon.bma.model.cluster.Resi- |
| 0.00083 | ref.negative.21:45658474, | | | | | | | | | | dent.macrophages.RDS |
| 8e−05 | ref.negative.14:98734567, | | | | | | | | | | |
| 0.00077 | ref.negative.16:6147645 | | | | | | | | | | |
| 0.00143 | ref.recessive.21:45658474, | 3 | 0.33333333 | 0.39215686 | 0.45692308 | 0.35294118 | 0.41176471 | 0.35294118 | 0.23076923 | 0.56 | colon.bma.model.cluster.Resi- |
| 8e−04 | ref.negative.21:45658474, | | | | | | | | | | dent.macrophages.RDS |
| 0.00046 | alt.negative.16:11331509, | | | | | | | | | | |
| 0.00014 | ref.negative.16:11331509, | | | | | | | | | | |
| 0.00078 | ref.negative.16:6147645 | | | | | | | | | | |
| 0.0163343877069926 | additive.9:117568766, | 6 | 0.39215686 | 0.56862745 | 0.54769231 | 0.6 | 0.5483871 | 0.6 | 0.46153846 | 0.68 | colon.bma.model.cluster.TA.RDS |
| 0.00808942351845002 | ref.negative.21:45658474, | | | | | | | | | | |
| 0.00743974801486341 | alt.negative.9:117568766, | | | | | | | | | | |
| 0.00469363596605493 | ref.negative.20:62305274, | | | | | | | | | | |
| 0.00130331622038961 | haploinsuff.16:11331509, | | | | | | | | | | |
| 0.0014161831522967 | ref.negative.14:98734567, | | | | | | | | | | |
| 0.00127458389711692 | ref.negative.5:11562221, | | | | | | | | | | |
| 0.0012985732970323 | alt.negative.16:11331509, | | | | | | | | | | |
| 0.00105750695921108 | ref.negative.16:11331509 | | | | | | | | | | |
| 0.01873 | additive.9:117568766, | 6 | 0.35294118 | 0.52941176 | 0.52153846 | 0.55555556 | 0.51515152 | 0.55555556 | 0.38461538 | 0.68 | colon.bma.model.cluster.TA.RDS |
| 0.01698 | alt.negative.9:117568766, | | | | | | | | | | |
| 0.04228 | ref.negative.21:45658474, | | | | | | | | | | |
| 0.0261 | ref.negative.20:62305274, | | | | | | | | | | |
| 0.02925 | ref.negative.16:11331509, | | | | | | | | | | |
| 0.03802 | alt.negative.16:11331509, | | | | | | | | | | |
| 0 | haploinsuff.16:11331509, | | | | | | | | | | |
| −0.01606 | ref.negative.14:98734567, | | | | | | | | | | |
| −0.02911 | ref.negative.5:11562221 | | | | | | | | | | |
| 0.04548 | alt.negative.16:11331509, | 5 | 0.37254902 | 0.54901961 | 0.50384615 | 0.57894737 | 0.53125 | 0.57894737 | 0.42307692 | 0.68 | colon.bma.model.cluster.TA.RDS |
| 0.01127 | ref.negative.21:45658474, | | | | | | | | | | |
| 0.03759 | ref.negative.20:62305274, | | | | | | | | | | |
| 0.04077 | ref.negative.16:11331509, | | | | | | | | | | |
| 0 | haploinsuff.16:11331509, | | | | | | | | | | |
| −0.02055 | ref.negative.14:98734567, | | | | | | | | | | |
| −0.02383 | ref.negative.5:11562221 | | | | | | | | | | |
| 0.02242 | additive.9:117568766, | 5 | 0.35294118 | 0.52941176 | 0.50153846 | 0.55555556 | 0.51515152 | 0.55555556 | 0.38461538 | 0.68 | colon.bma.model.cluster.TA.RDS |
| −5e−05 | alt.negative.9:117568766, | | | | | | | | | | |
| 0.03234 | ref.negative.21:45658474, | | | | | | | | | | |
| 0.01588 | ref.negative.20:62305274, | | | | | | | | | | |
| 0.02863 | ref.negative.16:11331509, | | | | | | | | | | |
| 0.03946 | alt.negative.16:11331509, | | | | | | | | | | |
| 0 | haploinsuff.16:11331509, | | | | | | | | | | |
| −0.01212 | ref.negative.14:98734567 | | | | | | | | | | |
| 0.01805 | additive.9:117568766, | 5 | 0.35294118 | 0.52941176 | 0.52 | 0.55555556 | 0.51515152 | 0.55555556 | 0.38461538 | 0.68 | colon.bma.model.cluster.TA.RDS |
| 0.01599 | alt.negative.9:117568766, | | | | | | | | | | |
| 0.03863 | ref.negative.21:45658474, | | | | | | | | | | |
| 0.02593 | ref.negative.20:62305274, | | | | | | | | | | |
| 0.02877 | ref.negative.16:11331509, | | | | | | | | | | |
| 0.03934 | alt.negative.16:11331509, | | | | | | | | | | |
| 0 | haploinsuff.16:11331509, | | | | | | | | | | |
| −0.03258 | ref.negative.5:11562221 | | | | | | | | | | |
| 0.02801 | additive.9:117568766, | 5 | 0.43137255 | 0.52941176 | 0.56230769 | 0.54545455 | 0.51724138 | 0.54545455 | 0.46153846 | 0.6 | colon.bma.model.cluster.TA.RDS |
| −0.0028 | alt.negative.9:117568766, | | | | | | | | | | |
| 0.0307 | ref.negative.21:45658474, | | | | | | | | | | |
| 0.01601 | ref.negative.20:62305274, | | | | | | | | | | |
| −0.02102 | ref.negative.14:98734567, | | | | | | | | | | |
| −0.04694 | ref.negative.5:11562221 | | | | | | | | | | |
| 0.0165 | additive.9:117568766, | 5 | 0.41176471 | 0.50980392 | 0.44384615 | 0.52380952 | 0.5 | 0.52380952 | 0.42307692 | 0.6 | colon.bma.model.cluster.TA.RDS |
| 0.02077 | alt.negative.9:117568766, | | | | | | | | | | |
| 0.01581 | ref.negative.20:62305274, | | | | | | | | | | |
| 0.02815 | ref.negative.16:11331509, | | | | | | | | | | |
| 0.0363 | alt.negative.16:11331509, | | | | | | | | | | |
| 0 | haploinsuff.16:11331509, | | | | | | | | | | |
| −0.01112 | ref.negative.14:98734567, | | | | | | | | | | |
| −0.01683 | ref.negative.5:11562221 | | | | | | | | | | |
| 0.019 | additive.9:117568766, | 5 | 0.39215686 | 0.49019608 | 0.49923077 | 0.5 | 0.48387097 | 0.5 | 0.38461538 | 0.6 | colon.bma.model.cluster.TA.RDS |
| 0.01624 | alt.negative.9:117568766, | | | | | | | | | | |
| 0.0365 | ref.negative.21:45658474, | | | | | | | | | | |
| 0.0252 | ref.negative.16:11331509, | | | | | | | | | | |
| 0.03571 | alt.negative.16:11331509, | | | | | | | | | | |
| 0 | haploinsuff.16:11331509, | | | | | | | | | | |
| −0.01213 | ref.negative.14:98734567, | | | | | | | | | | |
| −0.04116 | ref.negative.5:11562221 | | | | | | | | | | |
| 0.04479 | ref.negative.21:45658474, | 4 | 0.37254902 | 0.54901961 | 0.51615385 | 0.57894737 | 0.53125 | 0.57894737 | 0.42307692 | 0.68 | colon.bma.model.cluster.TA.RDS |
| 0.00941 | ref.negative.20:62305274, | | | | | | | | | | |
| 0.03207 | ref.negative.16:11331509, | | | | | | | | | | |
| 0.03668 | alt.negative.16:11331509, | | | | | | | | | | |
| 0 | haploinsuff.16:11331509, | | | | | | | | | | |
| −0.01347 | ref.negative.14:98734567 | | | | | | | | | | |
| 0.04941 | ref.negative.21:45658474, | 4 | 0.37254902 | 0.54901961 | 0.53923077 | 0.57894737 | 0.53125 | 0.57894737 | 0.42307692 | 0.68 | colon.bma.model.cluster.TA.RDS |
| 0.01496 | ref.negative.20:62305274, | | | | | | | | | | |
| 0.03783 | ref.negative.16:11331509, | | | | | | | | | | |
| 0.03972 | alt.negative.16:11331509, | | | | | | | | | | |
| 0 | haploinsuff.16:11331509, | | | | | | | | | | |
| −0.02962 | ref.negative.5:11562221 | | | | | | | | | | |
| 0.04111 | ref.negative.21:45658474, | 4 | 0.78431373 | 0.60784314 | 0.55923077 | 0.575 | 0.72727273 | 0.575 | 0.88461538 | 0.32 | colon.bma.model.cluster.TA.RDS |
| 0.01004 | ref.negative.20:62305274, | | | | | | | | | | |
| −0.02069 | ref.negative.14:98734567, | | | | | | | | | | |
| −0.03623 | ref.negative.5:11562221 | | | | | | | | | | |
| 0.02598 | additive.9:117568766, | 4 | 0.41176471 | 0.54901961 | 0.54076923 | 0.57142857 | 0.53333333 | 0.57142857 | 0.46153846 | 0.64 | colon.bma.model.cluster.TA.RDS |
| −0.00553 | alt.negative.9:117568766, | | | | | | | | | | |
| 0.02595 | ref.negative.21:45658474, | | | | | | | | | | |
| 0.02097 | ref.negative.20:62305274, | | | | | | | | | | |
| −0.02324 | ref.negative.14:98734567 | | | | | | | | | | |
| 0.02486 | additive.9:117568766, | 4 | 0.45098039 | 0.54901961 | 0.54230769 | 0.56521739 | 0.53571429 | 0.56521739 | 0.5 | 0.6 | colon.bma.model.cluster.TA.RDS |
| 0.00305 | alt.negative.9:117568766, | | | | | | | | | | |
| 0.03017 | ref.negative.21:45658474, | | | | | | | | | | |
| −0.02395 | ref.negative.14:98734567, | | | | | | | | | | |
| −0.04503 | ref.negative.5:11562221 | | | | | | | | | | |
| 0.02093 | additive.9:117568766, | 4 | 0.35294118 | 0.52941176 | 0.53692308 | 0.55555556 | 0.51515152 | 0.55555556 | 0.38461538 | 0.68 | colon.bma.model.cluster.TA.RDS |
| 1e−04 | alt.negative.9:117568766, | | | | | | | | | | |
| 0.03561 | ref.negative.21:45658474, | | | | | | | | | | |
| 0.02661 | ref.negative.20:62305274, | | | | | | | | | | |
| 0.03164 | ref.negative.16:11331509, | | | | | | | | | | |
| 0.03944 | alt.negative.16:11331509, | | | | | | | | | | |
| 0 | haploinsuff.16:11331509 | | | | | | | | | | |
| 0.02683 | additive.9:117568766, | 4 | 0.43137255 | 0.52941176 | 0.52153846 | 0.54545455 | 0.51724138 | 0.54545455 | 0.46153846 | 0.6 | colon.bma.model.cluster.TA.RDS |
| 0.00185 | alt.negative.9:117568766, | | | | | | | | | | |
| 0.02497 | ref.negative.21:45658474, | | | | | | | | | | |
| 0.01841 | ref.negative.20:62305274, | | | | | | | | | | |
| −0.04414 | ref.negative.5:11562221 | | | | | | | | | | |
| 0.0221 | additive.9:117568766, | 4 | 0.41176471 | 0.50980392 | 0.47230769 | 0.52380952 | 0.5 | 0.52380952 | 0.42307692 | 0.6 | colon.bma.model.cluster.TA.RDS |
| 0.00125 | alt.negative.9:117568766, | | | | | | | | | | |
| 0.02512 | ref.negative.20:62305274, | | | | | | | | | | |
| 0.02531 | ref.negative.16:11331509, | | | | | | | | | | |
| 0.03575 | alt.negative.16:11331509, | | | | | | | | | | |
| 0 | haploinsuff.16:11331509, | | | | | | | | | | |
| −0.01056 | ref.negative.14:98734567 | | | | | | | | | | |
| 0.02369 | additive.9:117568766, | 4 | 0.41176471 | 0.50980392 | 0.48692308 | 0.52380952 | 0.5 | 0.52380952 | 0.42307692 | 0.6 | colon.bma.model.cluster.TA.RDS |
| 0.00751 | alt.negative.9:117568766, | | | | | | | | | | |
| 0.01591 | ref.negative.20:62305274, | | | | | | | | | | |
| 0.02812 | ref.negative.16:11331509, | | | | | | | | | | |
| 0.03689 | alt.negative.16:11331509, | | | | | | | | | | |
| 0 | haploinsuff.16:11331509, | | | | | | | | | | |
| −0.01956 | ref.negative.5:11562221 | | | | | | | | | | |
| 0.04966 | ref.negative.21:45658474, | 4 | 0.41176471 | 0.50980392 | 0.51076923 | 0.52380952 | 0.5 | 0.52380952 | 0.42307692 | 0.6 | colon.bma.model.cluster.TA.RDS |
| 0.03281 | ref.negative.16:11331509, | | | | | | | | | | |
| 0.03509 | alt.negative.16:11331509, | | | | | | | | | | |
| 0 | haploinsuff.16:11331509, | | | | | | | | | | |
| −0.00885 | ref.negative.14:98734567, | | | | | | | | | | |
| −0.02447 | ref.negative.5:11562221 | | | | | | | | | | |
| 0.01004 | ref.negative.20:62305274, | 4 | 0.45098039 | 0.50980392 | 0.43615385 | 0.52173913 | 0.5 | 0.52173913 | 0.46153846 | 0.56 | colon.bma.model.cluster.TA.RDS |
| 0.03192 | ref.negative.16:11331509, | | | | | | | | | | |
| 0.03623 | alt.negative.16:11331509, | | | | | | | | | | |
| 0 | haploinsuff.16:11331509, | | | | | | | | | | |
| −0.02396 | ref.negative.14:98734567, | | | | | | | | | | |
| −0.00932 | ref.negative.5:11562221 | | | | | | | | | | |
| 0.01787 | additive.9:117568766, | 4 | 0.39215686 | 0.49019608 | 0.48538462 | 0.5 | 0.48387097 | 0.5 | 0.38461538 | 0.6 | colon.bma.model.cluster.TA.RDS |
| 0.00441 | alt.negative.9:117568766, | | | | | | | | | | |
| 0.03489 | ref.negative.21:45658474, | | | | | | | | | | |
| 0.0313 | ref.negative.16:11331509, | | | | | | | | | | |
| 0.03801 | alt.negative.16:11331509, | | | | | | | | | | |
| 0 | haploinsuff.16:11331509, | | | | | | | | | | |
| −0.01943 | ref.negative.14:98734567 | | | | | | | | | | |
| 0.01848 | additive.9:117568766, | 4 | 0.39215686 | 0.49019608 | 0.51 | 0.5 | 0.48387097 | 0.5 | 0.38461538 | 0.6 | colon.bma.model.cluster.TA.RDS |
| 0.01369 | alt.negative.9:117568766, | | | | | | | | | | |
| 0.03776 | ref.negative.21:45658474, | | | | | | | | | | |
| 0.0281 | ref.negative.16:11331509, | | | | | | | | | | |
| 0.03644 | alt.negative.16:11331509, | | | | | | | | | | |
| 0 | haploinsuff.16:11331509, | | | | | | | | | | |
| −0.02749 | ref.negative.5:11562221 | | | | | | | | | | |
| 0.02795 | additive.9:117568766, | 4 | 0.52941176 | 0.47058824 | 0.48538462 | 0.48148148 | 0.45833333 | 0.48148148 | 0.5 | 0.44 | colon.bma.model.cluster.TA.RDS |
| 0.0023 | alt.negative.9:117568766, | | | | | | | | | | |
| 0.0214 | ref.negative.20:62305274, | | | | | | | | | | |
| −0.01133 | ref.negative.14:98734567, | | | | | | | | | | |
| −0.03057 | ref.negative.5:11562221 | | | | | | | | | | |
| 0.02253 | additive.9:117568766, | 4 | 0.45098039 | 0.47058824 | 0.45615385 | 0.47826087 | 0.46428571 | 0.47826087 | 0.42307692 | 0.52 | colon.bma.model.cluster.TA.RDS |
| 0.00934 | alt.negative.9:117568766, | | | | | | | | | | |
| 0.02428 | ref.negative.16:11331509, | | | | | | | | | | |
| 0.03449 | alt.negative.16:11331509, | | | | | | | | | | |
| 0 | haploinsuff.16:11331509, | | | | | | | | | | |
| −0.0052 | ref.negative.14:98734567, | | | | | | | | | | |
| −0.02086 | ref.negative.5:11562221 | | | | | | | | | | |
| 0.02557 | additive.9:117568766, | 3 | 0.43137255 | 0.56862745 | 0.52076923 | 0.59090909 | 0.55172414 | 0.59090909 | 0.5 | 0.64 | colon.bma.model.cluster.TA.RDS |
| −0.0109 | alt.negative.9:117568766, | | | | | | | | | | |
| 0.02369 | ref.negative.21:45658474, | | | | | | | | | | |
| −0.02002 | ref.negative.14:98734567 | | | | | | | | | | |
| 0.04439 | ref.negative.21:45658474, | 3 | 0.37254902 | 0.54901961 | 0.55230769 | 0.57894737 | 0.53125 | 0.57894737 | 0.42307692 | 0.68 | colon.bma.model.cluster.TA.RDS |
| 0.00713 | ref.negative.20:62305274, | | | | | | | | | | |
| 0.03424 | ref.negative.16:11331509, | | | | | | | | | | |
| 0.03888 | alt.negative.16:11331509, | | | | | | | | | | |
| 0 | haploinsuff.16:11331509 | | | | | | | | | | |
| 0.0376 | ref.negative.21:45658474, | 3 | 0.78431373 | 0.60784314 | 0.57769231 | 0.575 | 0.72727273 | 0.575 | 0.88461538 | 0.32 | colon.bma.model.cluster.TA.RDS |
| 0.01965 | ref.negative.20:62305274, | | | | | | | | | | |
| −0.01636 | ref.negative.14:98734567 | | | | | | | | | | |
| 0.04169 | ref.negative.21:45658474, | 3 | 0.78431373 | 0.60784314 | 0.58384615 | 0.575 | 0.72727273 | 0.575 | 0.88461538 | 0.32 | colon.bma.model.cluster.TA.RDS |
| 0.0113 | ref.negative.20:62305274, | | | | | | | | | | |
| −0.03932 | ref.negative.5:11562221 | | | | | | | | | | |
| 0.02369 | additive.9:117568766, | 3 | 0.41176471 | 0.54901961 | 0.59230769 | 0.57142857 | 0.53333333 | 0.57142857 | 0.46153846 | 0.64 | colon.bma.model.cluster.TA.RDS |
| −0.00928 | alt.negative.9:117568766, | | | | | | | | | | |
| 0.02433 | ref.negative.21:45658474, | | | | | | | | | | |
| 0.02431 | ref.negative.20:62305274 | | | | | | | | | | |
| 0.02623 | additive.9:117568766, | 3 | 0.45098039 | 0.54901961 | 0.55153846 | 0.56521739 | 0.53571429 | 0.56521739 | 0.5 | 0.6 | colon.bma.model.cluster.TA.RDS |
| −0.0019 | alt.negative.9:117568766, | | | | | | | | | | |
| 0.03217 | ref.negative.21:45658474, | | | | | | | | | | |
| −0.04268 | ref.negative.5:11562221 | | | | | | | | | | |
| 0.04262 | ref.negative.21:45658474, | 3 | 0.8627451 | 0.56862745 | 0.51923077 | 0.54545455 | 0.71428571 | 0.54545455 | 0.92307692 | 0.2 | colon.bma.model.cluster.TA.RDS |
| −0.01736 | ref.negative.14:98734567, | | | | | | | | | | |
| −0.04466 | ref.negative.5:11562221 | | | | | | | | | | |
| 0.01039 | ref.negative.20:62305274, | 3 | 0.92156863 | 0.54901961 | 0.48 | 0.53191489 | 0.75 | 0.53191489 | 0.96153846 | 0.12 | colon.bma.model.cluster.TA.RDS |
| −0.01066 | ref.negative.14:98734567, | | | | | | | | | | |
| −0.01939 | ref.negative.5:11562221 | | | | | | | | | | |
| 0.02279 | additive.9:117568766, | 3 | 0.41176471 | 0.50980392 | 0.49076923 | 0.52380952 | 0.5 | 0.52380952 | 0.42307692 | 0.6 | colon.bma.model.cluster.TA.RDS |
| 0.00938 | alt.negative.9:117568766, | | | | | | | | | | |
| 0.01695 | ref.negative.20:62305274, | | | | | | | | | | |
| 0.03255 | ref.negative.16:11331509, | | | | | | | | | | |
| 0.03701 | alt.negative.16:11331509, | | | | | | | | | | |
| 0 | haploinsuff.16:11331509 | | | | | | | | | | |
| 0.04119 | ref.negative.21:45658474, | 3 | 0.41176471 | 0.50980392 | 0.5 | 0.52380952 | 0.5 | 0.52380952 | 0.42307692 | 0.6 | colon.bma.model.cluster.TA.RDS |
| 0.03116 | ref.negative.16:11331509, | | | | | | | | | | |
| 0.03389 | alt.negative.16:11331509, | | | | | | | | | | |
| 0 | haploinsuff.16:11331509, | | | | | | | | | | |
| −0.01866 | ref.negative.14:98734567 | | | | | | | | | | |
| 0.04545 | ref.negative.21:45658474, | 3 | 0.41176471 | 0.50980392 | 0.52923077 | 0.52380952 | 0.5 | 0.52380952 | 0.42307692 | 0.6 | colon.bma.model.cluster.TA.RDS |
| 0.0353 | ref.negative.16:11331509, | | | | | | | | | | |
| 0.03668 | alt.negative.16:11331509, | | | | | | | | | | |
| 0 | haploinsuff.16:11331509, | | | | | | | | | | |
| −0.02195 | ref.negative.5:11562221 | | | | | | | | | | |
| 0.01272 | ref.negative.20:62305274, | 3 | 0.45098039 | 0.50980392 | 0.44461538 | 0.52173913 | 0.5 | 0.52173913 | 0.46153846 | 0.56 | colon.bma.model.cluster.TA.RDS |
| 0.03412 | ref.negative.16:11331509, | | | | | | | | | | |
| 0.03831 | alt.negative.16:11331509, | | | | | | | | | | |
| 0 | haploinsuff.16:11331509, | | | | | | | | | | |
| −0.01943 | ref.negative.14:98734567 | | | | | | | | | | |
| 0.01426 | ref.negative.20:62305274, | 3 | 0.45098039 | 0.50980392 | 0.45923077 | 0.52173913 | 0.5 | 0.52173913 | 0.46153846 | 0.56 | colon.bma.model.cluster.TA.RDS |
| 0.03362 | ref.negative.16:11331509, | | | | | | | | | | |
| 0.03653 | alt.negative.16:11331509, | | | | | | | | | | |
| 0 | haploinsuff.16:11331509, | | | | | | | | | | |
| −0.01057 | ref.negative.5:11562221 | | | | | | | | | | |
| 0.01617 | additive.9:117568766, | 3 | 0.39215686 | 0.49019608 | 0.52384615 | 0.5 | 0.48387097 | 0.5 | 0.38461538 | 0.6 | colon.bma.model.cluster.TA.RDS |
| 0.00696 | alt.negative.9:117568766, | | | | | | | | | | |
| 0.03846 | ref.negative.21:45658474, | | | | | | | | | | |
| 0.0363 | ref.negative.16:11331509, | | | | | | | | | | |
| 0.03842 | alt.negative.16:11331509, | | | | | | | | | | |
| 0 | haploinsuff.16:11331509 | | | | | | | | | | |
| 0.02866 | additive.9:117568766, | 3 | 0.50980392 | 0.49019608 | 0.52 | 0.5 | 0.48 | 0.5 | 0.5 | 0.48 | colon.bma.model.cluster.TA.RDS |
| −0.01668 | alt.negative.9:117568766, | | | | | | | | | | |
| 0.01981 | ref.negative.20:62305274, | | | | | | | | | | |
| −0.00871 | ref.negative.14:98734567 | | | | | | | | | | |
| 0.0322 | additive.9:117568766, | 3 | 0.54901961 | 0.49019608 | 0.47076923 | 0.5 | 0.47826087 | 0.5 | 0.53846154 | 0.44 | colon.bma.model.cluster.TA.RDS |
| −0.00827 | alt.negative.9:117568766, | | | | | | | | | | |
| −0.01702 | ref.negative.14:98734567, | | | | | | | | | | |
| −0.04295 | ref.negative.5:11562221 | | | | | | | | | | |
| 0.02959 | additive.9:117568766, | 3 | 0.52941176 | 0.47058824 | 0.49461538 | 0.48148148 | 0.45833333 | 0.48148148 | 0.5 | 0.44 | colon.bma.model.cluster.TA.RDS |
| −0.00234 | alt.negative.9:117568766, | | | | | | | | | | |
| 0.024 | ref.negative.20:62305274, | | | | | | | | | | |
| −0.0379 | ref.negative.5:11562221 | | | | | | | | | | |
| 0.03024 | ref.negative.16:11331509, | 3 | 0.49019608 | 0.47058824 | 0.42 | 0.48 | 0.46153846 | 0.48 | 0.46153846 | 0.48 | colon.bma.model.cluster.TA.RDS |
| 0.03651 | alt.negative.16:11331509, | | | | | | | | | | |
| 0 | haploinsuff.16:11331509, | | | | | | | | | | |
| −0.02075 | ref.negative.14:98734567, | | | | | | | | | | |
| −0.02152 | ref.negative.5:11562221 | | | | | | | | | | |
| 0.02273 | additive.9:117568766, | 3 | 0.45098039 | 0.47058824 | 0.45461538 | 0.47826087 | 0.46428571 | 0.47826087 | 0.42307692 | 0.52 | colon.bma.model.cluster.TA.RDS |
| 0.01737 | alt.negative.9:117568766, | | | | | | | | | | |
| 0.02963 | ref.negative.16:11331509, | | | | | | | | | | |
| 0.03714 | alt.negative.16:11331509, | | | | | | | | | | |
| 0 | haploinsuff.16:11331509, | | | | | | | | | | |
| −0.03062 | ref.negative.5:11562221 | | | | | | | | | | |
| 0.02158 | additive.9:117568766, | 3 | 0.35294118 | 0.45098039 | 0.43230769 | 0.44444444 | 0.45454545 | 0.44444444 | 0.30769231 | 0.6 | colon.bma.model.cluster.TA.RDS |
| 0.00819 | alt.negative.9:117568766, | | | | | | | | | | |
| 0.02014 | ref.negative.16:11331509, | | | | | | | | | | |
| 0.03569 | alt.negative.16:11331509, | | | | | | | | | | |
| 0 | haploinsuff.16:11331509, | | | | | | | | | | |
| −0.01967 | ref.negative.14:98734567 | | | | | | | | | | |
| 0 | ref.negative.14:98734567, | 8 | 0.31372549 | 0.37254902 | 0.41307692 | 0.3125 | 0.4 | 0.3125 | 0.19230769 | 0.56 | ileum.bma.model.clus- |
| −0.00202 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04818 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03299 | ref.recessive.21:45658474, | | | | | | | | | | |
| 0 | alt.negative.21:45658474, | | | | | | | | | | |
| −0.02387 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05454 | ref.negative.20:62305274, | | | | | | | | | | |
| 0 | alt.recessive.20:62305274, | | | | | | | | | | |
| 0.02664 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0644 | ref.negative.16:11331509, | | | | | | | | | | |
| 0 | alt.recessive.16:11331509, | | | | | | | | | | |
| −0.01909 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07515 | alt.negative.9:117568766, | | | | | | | | | | |
| 0.01628 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01511 | alt.negative.16:6147645, | | | | | | | | | | |
| −0.01648 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01402 | alt.negative.11:128443099, | | | | | | | | | | |
| −0.02586 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.31372549 | 0.49019608 | 0.41076923 | 0.5 | 0.48571429 | 0.5 | 0.30769231 | 0.68 | ileum.bma.model.clus- |
| −0.00786 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05768 | ref.recessive.21:45658474, | | | | | | | | | | |
| 0 | alt.negative.21:45658474, | | | | | | | | | | |
| −0.01152 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07157 | ref.negative.20:62305274, | | | | | | | | | | |
| 0 | alt.recessive.20:62305274, | | | | | | | | | | |
| 0.0271 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06157 | ref.negative.16:11331509, | | | | | | | | | | |
| 0 | alt.recessive.16:11331509, | | | | | | | | | | |
| −0.00483 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08969 | alt.negative.9:117568766, | | | | | | | | | | |
| 0.02494 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| −0.01068 | alt.negative.16:6147645, | | | | | | | | | | |
| −0.0159 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00334 | alt.negative.11:128443099, | | | | | | | | | | |
| −0.0198 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.31372549 | 0.45098039 | 0.43923077 | 0.4375 | 0.45714286 | 0.4375 | 0.26923077 | 0.64 | ileum.bma.model.clus- |
| −0.00795 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05112 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0649 | ref.recessive.21:45658474, | | | | | | | | | | |
| 0 | alt.negative.21:45658474, | | | | | | | | | | |
| −0.01046 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06853 | ref.negative.16:11331509, | | | | | | | | | | |
| 0 | alt.recessive.16:11331509, | | | | | | | | | | |
| −0.02529 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07666 | alt.negative.9:117568766, | | | | | | | | | | |
| 0.01568 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01058 | alt.negative.16:6147645, | | | | | | | | | | |
| −0.01916 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01354 | alt.negative.11:128443099, | | | | | | | | | | |
| −0.02957 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.35294118 | 0.41176471 | 0.41615385 | 0.38888889 | 0.42424242 | 0.38888889 | 0.26923077 | 0.56 | ileum.bma.model.clus- |
| −0.01547 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03317 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05696 | ref.recessive.21:45658474, | | | | | | | | | | |
| 0 | alt.negative.21:45658474, | | | | | | | | | | |
| −0.0101 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06178 | ref.negative.20:62305274, | | | | | | | | | | |
| 0 | alt.recessive.20:62305274, | | | | | | | | | | |
| 0.02073 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06285 | ref.negative.16:11331509, | | | | | | | | | | |
| 0 | alt.recessive.16:11331509, | | | | | | | | | | |
| −0.01332 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −8e−04 | alt.negative.16:6147645, | | | | | | | | | | |
| −0.01442 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00294 | alt.negative.11:128443099, | | | | | | | | | | |
| −0.02513 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.31372549 | 0.41176471 | 0.41692308 | 0.375 | 0.42857143 | 0.375 | 0.23076923 | 0.6 | ileum.bma.model.clus- |
| −0.00919 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04431 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06012 | ref.recessive.21:45658474, | | | | | | | | | | |
| 0 | alt.negative.21:45658474, | | | | | | | | | | |
| −0.0063 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05655 | ref.negative.20:62305274, | | | | | | | | | | |
| 0 | alt.recessive.20:62305274, | | | | | | | | | | |
| 0.02069 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07025 | ref.negative.16:11331509, | | | | | | | | | | |
| 0 | alt.recessive.16:11331509, | | | | | | | | | | |
| −0.02276 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09837 | alt.negative.9:117568766, | | | | | | | | | | |
| 0.01222 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02106 | alt.negative.11:128443099, | | | | | | | | | | |
| −0.02318 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.31372549 | 0.41176471 | 0.39076923 | 0.375 | 0.42857143 | 0.375 | 0.23076923 | 0.6 | ileum.bma.model.clus- |
| −0.01202 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05118 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07399 | ref.negative.20:62305274, | | | | | | | | | | |
| 0 | alt.recessive.20:62305274, | | | | | | | | | | |
| 0.02307 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07266 | ref.negative.16:11331509, | | | | | | | | | | |
| 0 | alt.recessive.16:11331509, | | | | | | | | | | |
| −0.01233 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09746 | alt.negative.9:117568766, | | | | | | | | | | |
| 0.01877 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02402 | alt.negative.16:6147645, | | | | | | | | | | |
| −0.01761 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02679 | alt.negative.11:128443099, | | | | | | | | | | |
| −0.01023 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 7 | 0.31372549 | 0.41176471 | 0.41615385 | 0.375 | 0.42857143 | 0.375 | 0.23076923 | 0.6 | ileum.bma.model.clus- |
| 0.05311 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.02547 | ref.recessive.21:45658474, | | | | | | | | | | |
| 0 | alt.negative.21:45658474, | | | | | | | | | | |
| −0.0278 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0635 | ref.negative.20:62305274, | | | | | | | | | | |
| 0 | alt.recessive.20:62305274, | | | | | | | | | | |
| 0.02175 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07842 | ref.negative.16:11331509, | | | | | | | | | | |
| 0 | alt.recessive.16:11331509, | | | | | | | | | | |
| −0.02405 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08346 | alt.negative.9:117568766, | | | | | | | | | | |
| 0.01713 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 1e−04 | alt.negative.16:6147645, | | | | | | | | | | |
| −0.01695 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.0083 | alt.negative.11:128443099, | | | | | | | | | | |
| −0.02238 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 7 | 0.33333333 | 0.39215686 | 0.40538462 | 0.35294118 | 0.41176471 | 0.35294118 | 0.23076923 | 0.56 | ileum.bma.model.clus- |
| −0.01269 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04165 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03038 | ref.recessive.21:45658474, | | | | | | | | | | |
| 0 | alt.negative.21:45658474, | | | | | | | | | | |
| −0.0326 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07453 | ref.negative.20:62305274, | | | | | | | | | | |
| 0 | alt.recessive.20:62305274, | | | | | | | | | | |
| 0.02602 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06937 | ref.negative.16:11331509, | | | | | | | | | | |
| 0 | alt.recessive.16:11331509, | | | | | | | | | | |
| −0.0116 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06968 | alt.negative.9:117568766, | | | | | | | | | | |
| 0.0074 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02582 | alt.negative.16:6147645, | | | | | | | | | | |
| −0.02305 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive16:6147645, | | | | | | | | | | |
| 0 | haploinsuff.16:6147645 | | | | | | | | | | |
| 0 | ref.negative.14:98734567, | 7 | 0.31372549 | 0.37254902 | 0.43230769 | 0.3125 | 0.4 | 0.3125 | 0.19230769 | 0.56 | ileum.bma.model.clus- |
| −0.01095 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04006 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06296 | ref.recessive.21:45658474, | | | | | | | | | | |
| 0 | alt.negative.21:45658474, | | | | | | | | | | |
| 0.00141 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05578 | ref.negative.20:62305274, | | | | | | | | | | |
| 0 | alt.recessive.20:62305274, | | | | | | | | | | |
| 0.01547 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.09702 | alt.negative.9:117568766, | | | | | | | | | | |
| 0.02182 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00978 | alt.negative.16:6147645, | | | | | | | | | | |
| −0.01987 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01747 | alt.negative.11:128443099, | | | | | | | | | | |
| −0.02949 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.33333333 | 0.54901961 | 0.47692308 | 0.58823529 | 0.52941176 | 0.58823529 | 0.38461538 | 0.72 | ileum.bma.model.clus- |
| −0.00507 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05458 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06906 | ref.negative.16:11331509, | | | | | | | | | | |
| 0 | alt.recessive.16:11331509, | | | | | | | | | | |
| −0.0259 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09296 | alt.negative.9:117568766, | | | | | | | | | | |
| 0.03085 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01859 | alt.negative.16:6147645, | | | | | | | | | | |
| −0.01288 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.0196 | alt.negative.11:128443099, | | | | | | | | | | |
| −0.0131 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.49019608 | 0.44076923 | 0.5 | 0.48571429 | 0.5 | 0.3076921 | 0.68 | ileum.bma.model.clus- |
| −0.01456 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05671 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07334 | ref.negative.20:62305274, | | | | | | | | | | |
| 0 | alt.recessive.20:62305274, | | | | | | | | | | |
| 0.01356 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07085 | ref.negative.16:11331509, | | | | | | | | | | |
| 0 | alt.recessive.16:11331509, | | | | | | | | | | |
| −0.01359 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09073 | alt.negative.9:117568766, | | | | | | | | | | |
| 0.02505 | additive.9:117568766, | | | | | | | | | | |
| 0 | ref.negative.9:117568766, | | | | | | | | | | |
| 0 | haploinsuff.9:117568766, | | | | | | | | | | |
| 0 | aIt.recessive.9:117568766, | | | | | | | | | | |
| 0 | ref.recessive.9:117568766, | | | | | | | | | | |
| 0.02372 | alt.negative.11:128443099, | | | | | | | | | | |
| −0.01718 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.49019608 | 0.41692308 | 0.5 | 0.48571429 | 0.5 | 0.30769231 | 0.68 | ileum.bma.model.clus- |
| −0.01404 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.04912 | ref.recessive.21:45658474, | | | | | | | | | | |
| 0 | alt.negative.21:45658474, | | | | | | | | | | |
| −0.01746 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06796 | ref.negative.20:62305274, | | | | | | | | | | |
| 0 | alt.recessive.20:62305274, | | | | | | | | | | |
| 0.03259 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0695 | ref.negative.16:11331509, | | | | | | | | | | |
| 0 | alt.recessive.16:11331509, | | | | | | | | | | |
| −0.01616 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07936 | alt.negative.9:117568766, | | | | | | | | | | |
| 0.01591 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00419 | alt.negative.16:6147645, | | | | | | | | | | |
| −0.01758 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 6 | 0.31372549 | 0.49019608 | 0.46461538 | 0.5 | 0.48571429 | 0.5 | 0.30769231 | 0.68 | ileum.bma.model.clus- |
| −0.00823 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.03918 | ref.recessive.21:45658474, | | | | | | | | | | |
| 0 | alt.negative.21:45658474, | | | | | | | | | | |
| −0.02683 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05958 | ref.negative.16:11331509, | | | | | | | | | | |
| 0 | alt.recessive.16:11331509, | | | | | | | | | | |
| −0.02856 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.05574 | alt.negative.9:117568766, | | | | | | | | | | |
| 0.00616 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0211 | alt.negative.16:6147645, | | | | | | | | | | |
| −0.01985 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02096 | alt.negative.11:128443099, | | | | | | | | | | |
| −0.01244 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 6 | 0.31372549 | 0.49019608 | 0.46923077 | 0.5 | 0.48571429 | 0.5 | 0.30769231 | 0.68 | ileum.bma.model.clus- |
| 0.04755 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.034 | ref.recessive.21:45658474, | | | | | | | | | | |
| 0 | alt.negative.21:45658474, | | | | | | | | | | |
| −0.0285 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0726 | ref.negative.16:11331509, | | | | | | | | | | |
| 0 | alt.recessive.16:11331509, | | | | | | | | | | |
| −0.03345 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09042 | alt.negative.9:117568766, | | | | | | | | | | |
| 0.01449 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01875 | alt.negative.16:6147645, | | | | | | | | | | |
| −0.02079 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00806 | alt.negative.11:128443099, | | | | | | | | | | |
| −0.03305 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.33333333 | 0.47058824 | 0.45923077 | 0.47058824 | 0.47058824 | 0.47058824 | 0.30769231 | 0.64 | ileum.bma.model.clus- |
| −0.00926 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04967 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03126 | ref.recessive.21:45658474, | | | | | | | | | | |
| 0 | alt.negative.21:45658474, | | | | | | | | | | |
| −0.03091 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06709 | ref.negative.16:11331509, | | | | | | | | | | |
| 0 | alt.recessive.16:11331509, | | | | | | | | | | |
| −0.01892 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00862 | alt.negative.16:6147645, | | | | | | | | | | |
| −0.01477 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00988 | alt.negative.11:128443099, | | | | | | | | | | |
| −0.00745 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.45098039 | 0.41153846 | 0.4375 | 0.45714286 | 0.4375 | 0.26923077 | 0.64 | ileum.bma.model.clus- |
| −0.00793 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03498 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04335 | ref.recessive.21:45658474, | | | | | | | | | | |
| 0 | alt.negative.21:45658474, | | | | | | | | | | |
| −0.00891 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05055 | ref.negative.20:62305274, | | | | | | | | | | |
| 0 | alt.recessive.20:62305274, | | | | | | | | | | |
| 0.02046 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07294 | ref.negative.16:11331509, | | | | | | | | | | |
| 0 | alt.recessive.16:11331509, | | | | | | | | | | |
| −0.01431 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08572 | alt.negative.9:117568766, | | | | | | | | | | |
| 0.0147 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | refrecessive.9:117568766 | | | | | | | | | | |
| 0 | ref.negative.14:98734567, | 6 | 0.31372549 | 0.45098039 | 0.48692308 | 0.4375 | 0.45714286 | 0.4375 | 0.26923077 | 0.64 | ileum.bma.model.clus- |
| −0.01237 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0395 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03577 | ref.recessive.21:45658474, | | | | | | | | | | |
| 0 | alt.negative.21:45658474, | | | | | | | | | | |
| −0.02023 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05871 | ref.negative.16:11331509, | | | | | | | | | | |
| 0 | alt.recessive.16:11331509, | | | | | | | | | | |
| −0.01947 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07274 | alt.negative.9:117568766, | | | | | | | | | | |
| 0.01835 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01833 | alt.negative.16:6147645, | | | | | | | | | | |
| −0.02097 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 6 | 0.31372549 | 0.45098039 | 0.44846154 | 0.4375 | 0.45714286 | 0.4375 | 0.26923077 | 0.64 | ileum.bma.model.clus- |
| −0.00688 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04653 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0753 | ref.recessive.21:45658474, | | | | | | | | | | |
| 0 | alt.negative.21:45658474, | | | | | | | | | | |
| 0.00119 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0788 | ref.negative.16:11331509, | | | | | | | | | | |
| 0 | alt.recessive.16:11331509, | | | | | | | | | | |
| −0.03527 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06454 | alt.negative.9:117568766, | | | | | | | | | | |
| 0.00376 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01297 | alt.negative.11:128443099, | | | | | | | | | | |
| −0.02457 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.45098039 | 0.37615385 | 0.4375 | 0.45714286 | 0.4375 | 0.26923077 | 0.64 | ileum.bma.model.clus- |
| −0.01604 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04798 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07824 | ref.negative.20:62305274, | | | | | | | | | | |
| 0 | alt.recessive.20:62305274, | | | | | | | | | | |
| 0.0214 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0657 | ref.negative.16:11331509, | | | | | | | | | | |
| 0 | alt.recessive.16:11331509, | | | | | | | | | | |
| −0.00258 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08497 | alt.negative.9:117568766, | | | | | | | | | | |
| 0.02067 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02398 | alt.negative.16:6147645, | | | | | | | | | | |
| −0.01894 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 6 | 0.31372549 | 0.45098039 | 0.39538462 | 0.4375 | 0.45714286 | 0.4375 | 0.26923077 | 0.6 | ileum.bma.model.clus- |
| −0.02208 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04558 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.051 | ref.negative.20:62305274, | | | | | | | | | | |
| 0 | alt.recessive.20:62305274, | | | | | | | | | | |
| 0.00835 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.09671 | alt.negative.9:117568766, | | | | | | | | | | |
| 0.02366 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.03481 | alt.negative.16:6147645, | | | | | | | | | | |
| −0.02218 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02821 | alt.negative.11:128443099, | | | | | | | | | | |
| −0.00629 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.45098039 | 0.41461538 | 0.4375 | 0.45714286 | 0.4375 | 0.26923077 | 0.64 | ileum.bma.model.clus- |
| −0.01369 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05736 | ref.recessive.21:45658474, | | | | | | | | | | |
| 0 | alt.negative.21:45658474, | | | | | | | | | | |
| −0.0126 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06364 | ref.negative.20:62305274, | | | | | | | | | | |
| 0 | alt.recessive.20:62305274, | | | | | | | | | | |
| 0.03237 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07378 | ref.negative.16:11331509, | | | | | | | | | | |
| 0 | alt.recessive.16:11331509, | | | | | | | | | | |
| −0.0178 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08616 | alt.negative.9:117568766, | | | | | | | | | | |
| 0.01525 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00452 | alt.negative.11:128443099, | | | | | | | | | | |
| −0.02057 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.45098039 | 0.42230769 | 0.4375 | 0.45714286 | 0.4375 | 0.26923077 | 0.64 | ileum.bma.model.clus- |
| −0.01856 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.03523 | ref.recessive.21:45658474, | | | | | | | | | | |
| 0 | alt.negative.21:45658474, | | | | | | | | | | |
| −0.02726 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07597 | ref.negative.20:62305274, | | | | | | | | | | |
| 0 | alt.recessive.20:62305274, | | | | | | | | | | |
| 0.02616 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05498 | ref.negative.16:11331509, | | | | | | | | | | |
| 0 | alt.recessive.16:11331509, | | | | | | | | | | |
| 0.00117 | alt.negative.16:11331509, | | | | | | | | | | |
| 0 | ref.recessive16:11331509, | | | | | | | | | | |
| 0 | haploinsuff.16:11331509, | | | | | | | | | | |
| 0 | additive.16:11331509, | | | | | | | | | | |
| −0.02285 | alt.negative.16:6147645, | | | | | | | | | | |
| −0.00967 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00261 | alt.negative.11:128443099, | | | | | | | | | | |
| −0.02372 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.04643 | ref.recessive.21:45658474, | 6 | 0.31372549 | 0.45098039 | 0.42461538 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.02732 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07174 | ref.negative.20:62305274, | | | | | | | | | | |
| 0 | alt.recessive.20:62305274, | | | | | | | | | | |
| 0.02999 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0742 | ref.negative.16:11331509, | | | | | | | | | | |
| 0 | alt.recessive.16:11331509, | | | | | | | | | | |
| −0.02419 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06873 | alt.negative.9:117568766, | | | | | | | | | | |
| 0.00838 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| −0.00029 | alt.negative.16:6147645, | | | | | | | | | | |
| −0.01445 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00179 | alt.negative.11:128443099, | | | | | | | | | | |
| −0.02358 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.33333333 | 0.43137255 | 0.44846154 | 0.41176471 | 0.44117647 | 0.41176471 | 0.26923077 | 0.6 | ileum.bma.model.clus- |
| −0.01743 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04157 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03317 | ref.recessive.21:45658474, | | | | | | | | | | |
| 0 | alt.negative.21:45658474, | | | | | | | | | | |
| −0.0273 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0519 | ref.negative.20:62305274, | | | | | | | | | | |
| 0 | alt.recessive.20:62305274, | | | | | | | | | | |
| 0.01391 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06111 | ref.negative.16:11331509, | | | | | | | | | | |
| 0 | alt.recessive.16:11331509, | | | | | | | | | | |
| −0.02673 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01078 | alt.negative.11:128443099, | | | | | | | | | | |
| −0.02466 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.33333333 | 0.43137255 | 0.43846154 | 0.41176471 | 0.44117647 | 0.41176471 | 0.26923077 | 0.6 | ileum.bma.model.clus- |
| −0.02308 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.03688 | ref.recessive.21:45658474, | | | | | | | | | | |
| 0 | alt.negative.21:45658474, | | | | | | | | | | |
| −0.02444 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05811 | ref.negative.20:62305274, | | | | | | | | | | |
| 0 | alt.recessive.20:62305274, | | | | | | | | | | |
| 0.02366 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07388 | alt.negative.9:117568766, | | | | | | | | | | |
| 0.01509 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| −0.0042 | alt.negative.16:6147645, | | | | | | | | | | |
| −0.01274 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.0069 | alt.negative.11:128443099, | | | | | | | | | | |
| −0.01427 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 6 | 0.33333333 | 0.43137255 | 0.41384615 | 0.41176471 | 0.44117647 | 0.41176471 | 0.26923077 | 0.6 | ileum.bma.model.clus- |
| 0.03804 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06046 | ref.recessive.21:45658474, | | | | | | | | | | |
| 0 | alt.negative.21:45658474, | | | | | | | | | | |
| −0.00591 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05333 | ref.negative.20:62305274, | | | | | | | | | | |
| 0 | alt.recessive.20:62305274, | | | | | | | | | | |
| 0.02385 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0615 | ref.negative.16:11331509, | | | | | | | | | | |
| 0 | alt.recessive.16:11331509, | | | | | | | | | | |
| −0.01399 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09637 | alt.negative.9:117568766, | | | | | | | | | | |
| 0.01944 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0185 | alt.negative.16:6147645, | | | | | | | | | | |
| −0.02821 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 6 | 0.33333333 | 0.43137255 | 0.39461538 | 0.41176471 | 0.44117647 | 0.41176471 | 0.26923077 | 0.6 | ileum.bma.model.clus- |
| 0.05429 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.09699 | ref.negative.20:62305274, | | | | | | | | | | |
| 0 | alt.recessive.20:62305274, | | | | | | | | | | |
| 0.02583 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07602 | ref.negative.l6:11331509, | | | | | | | | | | |
| 0 | alt.recessive.16:11331509, | | | | | | | | | | |
| −0.01547 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08937 | alt.negative.9:117568766, | | | | | | | | | | |
| 0.0245 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01308 | alt.negative.16:6147645, | | | | | | | | | | |
| −0.01422 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02877 | alt.negative.11:128443099, | | | | | | | | | | |
| −0.00017 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.35294118 | 0.41176471 | 0.41461538 | 0.38888889 | 0.42424242 | 0.38888889 | 0.26923077 | 0.56 | ileum.bma.model.clus- |
| −0.00942 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04332 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0194 | ref.recessive.21:45658474, | | | | | | | | | | |
| 0 | alt.negative.21:45658474, | | | | | | | | | | |
| −0.03729 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06871 | ref.negative.20:62305274, | | | | | | | | | | |
| 0 | alt.recessive.20:62305274, | | | | | | | | | | |
| 0.02172 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07294 | ref.negative.16:11331509, | | | | | | | | | | |
| 0 | alt.recessive.16:11331509, | | | | | | | | | | |
| −0.01505 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01973 | alt.negative.16:6147645, | | | | | | | | | | |
| −0.02357 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 6 | 0.35294118 | 0.41176471 | 0.37615385 | 0.38888889 | 0.42424242 | 0.38888889 | 0.26923077 | 0.56 | ileum.bma.model.clus- |
| −0.01965 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0487 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06856 | ref.negative.20:62305274, | | | | | | | | | | |
| 0 | alt.recessive.20:62305274, | | | | | | | | | | |
| 0.01322 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06602 | ref.negative.16:11331509, | | | | | | | | | | |
| 0 | alt.recessive.16:11331509, | | | | | | | | | | |
| −0.00637 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01875 | alt.negative.16:6147645, | | | | | | | | | | |
| −0.01279 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01694 | alt.negative.11:128443099, | | | | | | | | | | |
| 0.00063 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 6 | 0.31372549 | 0.41176471 | 0.42 | 0.375 | 0.42857143 | 0.375 | 0.23076923 | 0.6 | ileum.bma.model.clus- |
| 0.05105 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04908 | ref.recessive.21:45658474, | | | | | | | | | | |
| 0 | alt.negative.21:45658474, | | | | | | | | | | |
| −0.00543 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05357 | ref.negative.20:62305274, | | | | | | | | | | |
| 0 | alt.recessive.20:62305274, | | | | | | | | | | |
| 0.02065 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.09453 | alt.negative.9:117568766, | | | | | | | | | | |
| 0.02298 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01263 | alt.negative.16:6147645, | | | | | | | | | | |
| −0.02143 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01834 | alt.negative.11:128443099, | | | | | | | | | | |
| −0.01826 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.33333333 | 0.39215686 | 0.41923077 | 0.35294118 | 0.41176471 | 0.35294118 | 0.23076923 | 0.56 | ileum.bma.model.clus- |
| −0.01337 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03418 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05042 | ref.recessive.21:45658474, | | | | | | | | | | |
| 0 | alt.negative.21:45658474, | | | | | | | | | | |
| −0.01333 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04479 | ref.negative.20:62305274, | | | | | | | | | | |
| 0 | alt.recessive.20:62305274, | | | | | | | | | | |
| 0.0229 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0756 | alt.negative.9:117568766, | | | | | | | | | | |
| 0.01907 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02796 | alt.negative.16:6147645, | | | | | | | | | | |
| −0.02957 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 6 | 0.33333333 | 0.39215686 | 0.43615385 | 0.35294118 | 0.41176471 | 0.35294118 | 0.23076923 | 0.56 | ileum.bma.model.clus- |
| −0.01774 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04201 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05766 | ref.recessive.21:45658474, | | | | | | | | | | |
| 0 | alt.negative.21:45658474, | | | | | | | | | | |
| 0.00344 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06236 | ref.negative.20:62305274, | | | | | | | | | | |
| 0 | alt.recessive.20:62305274, | | | | | | | | | | |
| 0.01227 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08845 | alt.negative.9:117568766, | | | | | | | | | | |
| 0.02084 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01948 | alt.negative.11:128443099, | | | | | | | | | | |
| −0.01904 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.37254902 | 0.43769231 | 0.3125 | 0.4 | 0.3125 | 0.19230769 | 0.56 | ileum.bma.model.clus- |
| −0.01635 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.02907 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.02491 | ref.recessive.21:45658474, | | | | | | | | | | |
| 0 | alt.negative.21:45658474, | | | | | | | | | | |
| −0.042 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.03818 | ref.negative.20:62305274, | | | | | | | | | | |
| 0 | alt.recessive.20:62305274, | | | | | | | | | | |
| 0.0091 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01264 | alt.negative.16:6147645, | | | | | | | | | | |
| −0.0231 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01136 | alt.negative.11:128443099, | | | | | | | | | | |
| −0.01331 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.37254902 | 0.44230769 | 0.3125 | 0.4 | 0.3125 | 0.19230769 | 0.56 | ileum.bma.model.clus- |
| −0.01493 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04853 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0786 | ref.recessive.21:45658474, | | | | | | | | | | |
| 0 | alt.negative.21:45658474, | | | | | | | | | | |
| 0.00613 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0778 | alt.negative.9:117568766, | | | | | | | | | | |
| 0.01453 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00642 | alt.negative.16:6147645, | | | | | | | | | | |
| −0.01456 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01631 | alt.negative.11:128443099, | | | | | | | | | | |
| −0.02206 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 6 | 0.31372549 | 0.37254902 | 0.37769231 | 0.3125 | 0.4 | 0.3125 | 0.19230769 | 0.56 | ileum.bma.model.clus- |
| −0.01435 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.09156 | ref.negative.20:62305274, | | | | | | | | | | |
| 0 | alt.recessive.20:62305274, | | | | | | | | | | |
| 0.02408 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05945 | ref.negative.16:11331509, | | | | | | | | | | |
| 0 | alt.recessive.16:11331509, | | | | | | | | | | |
| −0.00027 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06289 | alt.negative.9:117568766, | | | | | | | | | | |
| 0.01777 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00176 | alt.negative.16:6147645, | | | | | | | | | | |
| −0.01319 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00672 | alt.negative.11:128443099, | | | | | | | | | | |
| −0.00564 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 6 | 0.31372549 | 0.37254902 | 0.40230769 | 0.3125 | 0.4 | 0.3125 | 0.19230769 | 0.56 | ileum.bma.model.clus- |
| 0.04839 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06626 | ref.recessive.21:45658474, | | | | | | | | | | |
| 0 | alt.negative.21:45658474, | | | | | | | | | | |
| 0.00308 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05976 | ref.negative.20:62305274, | | | | | | | | | | |
| 0 | alt.recessive.20:62305274, | | | | | | | | | | |
| 0.03106 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06364 | ref.negative.16:11331509, | | | | | | | | | | |
| 0 | alt.recessive.16:11331509, | | | | | | | | | | |
| −0.02766 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.10863 | alt.negative.9:117568766, | | | | | | | | | | |
| 0.01549 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01058 | alt.negative.11:128443099, | | | | | | | | | | |
| −0.02446 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 6 | 0.31372549 | 0.37254902 | 0.42846154 | 0.3125 | 0.4 | 0.3125 | 0.19230769 | 0.56 | ileum.bma.model.clus- |
| 0.04328 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.02499 | ref.recessive.21:45658474, | | | | | | | | | | |
| 0 | alt.negative.21:45658474, | | | | | | | | | | |
| −0.0396 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04981 | ref.negative.20:62305274, | | | | | | | | | | |
| 0 | alt.recessive.20:62305274, | | | | | | | | | | |
| 0.01508 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07196 | ref.negative.16:11331509, | | | | | | | | | | |
| 0 | alt.recessive.16:11331509, | | | | | | | | | | |
| −0.02342 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.0099 | alt.negative.16:6147645, | | | | | | | | | | |
| −0.01973 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00536 | alt.negative.11:128443099, | | | | | | | | | | |
| −0.02376 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.52941176 | 0.48230769 | 0.5625 | 0.51428571 | 0.5625 | 0.34615385 | 0.72 | ileum.bma.model.clus- |
| −0.01744 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05282 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.08848 | alt.negative.9:117568766, | | | | | | | | | | |
| 0.026 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02448 | alt.negative.16:6147645, | | | | | | | | | | |
| −0.01736 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02944 | alt.negative.11:128443099, | | | | | | | | | | |
| 0.00416 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00413167705741463 | alt.recessive.14:98734567, | 5 | 0.33333333 | 0.50980392 | 0.56923077 | 0.52941176 | 0.5 | 0.52941176 | 0.34615385 | 0.68 | ileum.bma.model.clus- |
| −0.0044359554292331 | alt.recessive.21:45658474, | | | | | | | | | | ter.Entero_Clonocyte.RDS |
| −0.00250017985876822 | alt.recessive.5:11562221, | | | | | | | | | | |
| 0.000650367063738027 | additive.9:117568766, | | | | | | | | | | |
| −0.000997363768312975 | additive.21:45658474, | | | | | | | | | | |
| 0.000657529290080377 | alt.negative.9:117568766, | | | | | | | | | | |
| −0.0011883988810037 | additive.5:11562221, | | | | | | | | | | |
| 0.000767215741330144 | haploinsuff.5:11562221, | | | | | | | | | | |
| −3.83886285000346e−05 | alt.negative.5:11562221, | | | | | | | | | | |
| 0.000308632211177067 | haploinsuff.21:45658474, | | | | | | | | | | |
| −0.000306844405921009 | ref.negative.9:117568766, | | | | | | | | | | |
| 0.000212437039615959 | alt.recessive.9:117568766, | | | | | | | | | | |
| 0.0001621570612628 | haploinsuff.9:117568766, | | | | | | | | | | |
| −0.000285045293093247 | ref.recessive.5:11562221, | | | | | | | | | | |
| −0.000243858655992485 | alt.negative.21:45658474, | | | | | | | | | | |
| −3.17901237909852e−06 | ref.recessive.21:45658474, | | | | | | | | | | |
| −0.000332281196715872 | ref.recessive.9:117568766, | | | | | | | | | | |
| 0.000647604076515593 | ref.negative.14:98734567, | | | | | | | | | | |
| 2.56340836167717e−05 | alt.negative.16:6147645, | | | | | | | | | | |
| 0.0003441190525664 | ref.negative.5:11562221, | | | | | | | | | | |
| 0.000194490403126353 | ref.negative.21:45658474 | | | | | | | | | | |
| −0.0048 | alt.recessive.14:98734567, | 5 | 0.33333333 | 0.50980392 | 0.55538462 | 0.52941176 | 0.5 | 0.52941176 | 0.34615385 | 0.68 | ileum.bma.model.clus- |
| 0 | ref.negative.14:98734567, | | | | | | | | | | ter.Entero_Clonocyte.RDS |
| −0.005 | alt.recessive.21:45658474, | | | | | | | | | | |
| −0.00093 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00462 | alt.recessive.5:11562221, | | | | | | | | | | |
| −0.00019 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00101 | additive.9:117568766, | | | | | | | | | | |
| 0.00076 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| 0.00016 | alt.negative.16:6147645 | | | | | | | | | | |
| 0 | ref.negative.14:98734567, | 5 | 0.37254902 | 0.50980392 | 0.48615385 | 0.52631579 | 0.5 | 0.52631579 | 0.38461538 | 0.64 | ileum.bma.model.clus- |
| −0.015 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0612 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05965 | ref.negative.16:11331509, | | | | | | | | | | |
| 0 | alt.recessive.16:11331509, | | | | | | | | | | |
| −0.02477 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09406 | alt.negative.9:117568766, | | | | | | | | | | |
| 0.02228 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.03312 | alt.negative.11:128443099, | | | | | | | | | | |
| −0.01295 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.49019608 | 0.48384615 | 0.5 | 0.48571429 | 0.5 | 0.30769231 | 0.68 | ileum.bma.model.clus- |
| −0.01255 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05125 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04432 | ref.recessive.21:45658474, | | | | | | | | | | |
| 0 | alt.negative.21:45658474, | | | | | | | | | | |
| −0.01644 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06678 | ref.negative.16:11331509, | | | | | | | | | | |
| 0 | alt.recessive.16:11331509, | | | | | | | | | | |
| −0.01927 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07275 | alt.negative.9:117568766, | | | | | | | | | | |
| 0.01397 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 5 | 0.35294118 | 0.49019608 | 0.43076923 | 0.5 | 0.48484848 | 0.5 | 0.34615385 | 0.64 | ileum.bma.model.clus- |
| −0.00786 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03857 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.07605 | ref.recessive.21:45658474, | | | | | | | | | | |
| 0 | alt.negative.21:45658474, | | | | | | | | | | |
| 0.00722 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07248 | alt.negative.9:117568766, | | | | | | | | | | |
| 0.01204 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0248 | alt.negative.16:6147645, | | | | | | | | | | |
| −0.0247 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.49019608 | 0.44230769 | 0.5 | 0.48571429 | 0.5 | 0.30769231 | 0.68 | ileum.bma.model.clus- |
| −0.01242 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05532 | ref.recessive.21:45658474, | | | | | | | | | | |
| 0 | alt.negative.21:45658474, | | | | | | | | | | |
| −0.01514 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04537 | ref.negative.20:62305274, | | | | | | | | | | |
| 0 | alt.recessive.20:62305274, | | | | | | | | | | |
| 0.02246 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06455 | ref.negative.16:11331509, | | | | | | | | | | |
| 0 | alt.recessive.16:11331509, | | | | | | | | | | |
| −0.02066 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.05933 | alt.negative.9:117568766, | | | | | | | | | | |
| 0.00086 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 5 | 0.31372549 | 0.49019608 | 0.39 | 0.5 | 0.48571429 | 0.5 | 0.30769231 | 0.68 | ileum.bma.model.clus- |
| −0.02665 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.07909 | ref.negative.20:62305274, | | | | | | | | | | |
| 0 | alt.recessive.20:62305274, | | | | | | | | | | |
| 0.00751 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.10068 | alt.negative.9:117568766, | | | | | | | | | | |
| 0.03173 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01515 | alt.negative.16:6147645, | | | | | | | | | | |
| −0.01057 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02873 | alt.negative.11:128443099, | | | | | | | | | | |
| 0.00024 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.37254902 | 0.47058824 | 0.44769231 | 0.47368421 | 0.46875 | 0.47368421 | 0.34615385 | 0.6 | ileum.bma.model.clus- |
| 0.04269 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.00729 | ref.recessive.21:45658474, | | | | | | | | | | |
| 0 | alt.negative.21:45658474, | | | | | | | | | | |
| −0.05181 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06846 | ref.negative.20:62305274, | | | | | | | | | | |
| 0 | alt.recessive.20:62305274, | | | | | | | | | | |
| 0.03016 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07382 | ref.negative.16:11331509, | | | | | | | | | | |
| 0 | alt.recessive.16:11331509, | | | | | | | | | | |
| −0.01541 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00414 | alt.negative.16:6147645, | | | | | | | | | | |
| −0.02148 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.33333333 | 0.47058824 | 0.40461538 | 0.47058824 | 0.47058824 | 0.47058824 | 0.30769231 | 0.64 | ileum.bma.model.clus- |
| −0.01008 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05194 | ref.recessive.21:45658474, | | | | | | | | | | |
| 0 | alt.negative.21:45658474, | | | | | | | | | | |
| −0.01003 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05245 | ref.negative.20:62305274, | | | | | | | | | | |
| 0 | alt.recessive.20:62305274, | | | | | | | | | | |
| 0.02595 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06512 | alt.negative.9:117568766, | | | | | | | | | | |
| 0.01373 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01282 | alt.negative.16:6147645, | | | | | | | | | | |
| −0.01608 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.35294118 | 0.45098039 | 0.41615385 | 0.44444444 | 0.45454545 | 0.44444444 | 0.30769231 | 0.6 | ileum.bma.model.clus- |
| −0.00467 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.043 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.08111 | ref.negative.20:62305274, | | | | | | | | | | |
| 0 | alt.recessive.20:62305274, | | | | | | | | | | |
| 0.01885 | ref.recessive.20:62305274, | | | | | | | | | | |
| 0 | alt.negative.20:62305274, | | | | | | | | | | |
| 0 | haploinsuff.20;62305274, | | | | | | | | | | |
| 0 | additive.20:62305274, | | | | | | | | | | |
| −0.0767 | ref.negative.16:11331509, | | | | | | | | | | |
| 0 | alt.recessive.16:11331509, | | | | | | | | | | |
| −0.01301 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09426 | alt.negative.9:117568766, | | | | | | | | | | |
| 0.02367 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 5 | 0.35294118 | 0.45098039 | 0.42 | 0.44444444 | 0.45454545 | 0.44444444 | 0.30769231 | 0.6 | ileum.bma.model.clus- |
| −0.01332 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05861 | ref.recessive.21:45658474, | | | | | | | | | | |
| 0 | alt.negative.21:45658474, | | | | | | | | | | |
| −0.00781 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07331 | ref.negative.20:62305274, | | | | | | | | | | |
| 0 | alt.recessive.20:62305274, | | | | | | | | | | |
| 0.03099 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05306 | ref.negative.16:11331509, | | | | | | | | | | |
| 0 | alt.recessive.16:11331509, | | | | | | | | | | |
| −0.00576 | alt.negative.16:11331509, | | | | | | | | | | |
| 0 | ref.recessive16:11331509, | | | | | | | | | | |
| 0 | haploinsuff.16:11331509, | | | | | | | | | | |
| 0 | additive.16:11331509, | | | | | | | | | | |
| −0.0109 | alt.negative.16:6147645, | | | | | | | | | | |
| −0.01598 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.45098039 | 0.43461538 | 0.4375 | 0.45714286 | 0.4375 | 0.26923077 | 0.64 | ileum.bma.model.clus- |
| −0.0133 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04722 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04508 | ref.recessive.21:45658474, | | | | | | | | | | |
| 0 | alt.negative.21:45658474, | | | | | | | | | | |
| −0.02039 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.03664 | ref.negative.20:62305274, | | | | | | | | | | |
| 0 | alt.recessive.20:62305274, | | | | | | | | | | |
| 0.02237 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06624 | ref.negative.16:11331509, | | | | | | | | | | |
| 0 | alt.recessive.16:11331509, | | | | | | | | | | |
| −0.01603 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 5 | 0.31372549 | 0.45098039 | 0.42230769 | 0.4375 | 0.45714286 | 0.4375 | 0.26923077 | 0.64 | ileum.bma.model.clus- |
| −0.01116 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04128 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.07622 | ref.recessive.21:45658474, | | | | | | | | | | |
| 0 | alt.negative.21:45658474, | | | | | | | | | | |
| 0.00261 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04384 | ref.negative.20:62305274, | | | | | | | | | | |
| 0 | alt.recessive.20:62305274, | | | | | | | | | | |
| 0.02406 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07208 | alt.negative.9:117568766, | | | | | | | | | | |
| 0.01235 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 5 | 0.31372549 | 0.45098039 | 0.46 | 0.4375 | 0.45714286 | 0.4375 | 0.26923077 | 0.64 | ileum.bma.model.clus- |
| −0.01158 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04999 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.02467 | ref.recessive.21:45658474, | | | | | | | | | | |
| 0 | alt.negative.21:45658474, | | | | | | | | | | |
| −0.03964 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05959 | ref.negative.16:11331509, | | | | | | | | | | |
| 0 | alt.recessive.16:11331509, | | | | | | | | | | |
| −0.01257 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.02101 | alt.negative.16:6147645, | | | | | | | | | | |
| −0.0203 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.45098039 | 0.45461538 | 0.4375 | 0.45714286 | 0.4375 | 0.26923077 | 0.64 | ileum.bma.model.clus- |
| −0.0092 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04011 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0575 | ref.recessive.21:45658474, | | | | | | | | | | |
| 0 | alt.negative.21:45658474, | | | | | | | | | | |
| −0.00787 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06953 | ref.negative.16:11331509, | | | | | | | | | | |
| 0 | alt.recessive.16:11331509, | | | | | | | | | | |
| −0.02394 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00853 | alt.negative.11:128443099, | | | | | | | | | | |
| −0.01293 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.45098039 | 0.37461538 | 0.4375 | 0.45714286 | 0.4375 | 0.26923077 | 0.64 | ileum.bma.model.clus- |
| −0.01719 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03733 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05779 | ref.negative.20:62305274, | | | | | | | | | | |
| 0 | alt.recessive.20:62305274, | | | | | | | | | | |
| 0.01537 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.09348 | alt.negative.9:117568766, | | | | | | | | | | |
| 0.03261 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.03145 | alt.negative.16:6147645, | | | | | | | | | | |
| −0.02444 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.45098039 | 0.45384615 | 0.4375 | 0.45714286 | 0.4375 | 0.26923077 | 0.64 | ileum.bma.model.clus- |
| −0.0153 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05191 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07184 | ref.negative.16:11331509, | | | | | | | | | | |
| 0 | alt.recessive.16:11331509, | | | | | | | | | | |
| −0.01354 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07963 | alt.negative.9:117568766, | | | | | | | | | | |
| 0.02378 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.04522 | alt.negative.16:6147645, | | | | | | | | | | |
| −0.01993 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.45098039 | 0.45230769 | 0.4375 | 0.45714286 | 0.4375 | 0.26923077 | 0.64 | ileum.bma.model.clus- |
| −0.00583 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04651 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06509 | ref.negative.16:11331509, | | | | | | | | | | |
| 0 | alt.recessive.16:11331509, | | | | | | | | | | |
| −0.02229 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00927 | alt.negative.16:6147645, | | | | | | | | | | |
| −0.00867 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01608 | alt.negative.11:128443099, | | | | | | | | | | |
| −0.00749 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.45098039 | 0.43461538 | 0.4375 | 0.45714286 | 0.4375 | 0.26923077 | 0.64 | ileum.bma.model.clus- |
| −0.01762 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.06145 | ref.recessive.21:45658474, | | | | | | | | | | |
| 0 | alt.negative.21:45658474, | | | | | | | | | | |
| −0.01091 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04281 | ref.negative.20:62305274, | | | | | | | | | | |
| 0 | alt.recessive.20:62305274, | | | | | | | | | | |
| 0.01784 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06732 | ref.negative.16:11331509, | | | | | | | | | | |
| 0 | alt.recessive.16:11331509, | | | | | | | | | | |
| −0.02343 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00266 | alt.negative.11:128443099, | | | | | | | | | | |
| −0.02373 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.45098039 | 0.44846154 | 0.4375 | 0.45714286 | 0.4375 | 0.26923077 | 0.64 | ileum.bma.model.clus- |
| −0.00163 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.07807 | ref.recessive.21:45658474, | | | | | | | | | | |
| 0 | alt.negative.21:45658474, | | | | | | | | | | |
| 0.00432 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05993 | ref.negative.16:11331509, | | | | | | | | | | |
| 0 | alt.recessive.16:11331509, | | | | | | | | | | |
| −0.01796 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07458 | alt.negative.9:117568766, | | | | | | | | | | |
| 0.01229 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0119 | alt.negative.16:6147645, | | | | | | | | | | |
| −0.01777 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.31372549 | 0.45098039 | 0.45230769 | 0.4375 | 0.45714286 | 0.4375 | 0.26923077 | 0.64 | ileum.bma.model.clus- |
| −0.01073 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.07267 | ref.recessive.21:45658474, | | | | | | | | | | |
| 0 | alt.negative.21:45658474, | | | | | | | | | | |
| 0.00913 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05463 | ref.negative.16:11331509, | | | | | | | | | | |
| 0 | alt.recessive.16:11331509, | | | | | | | | | | |
| −0.02472 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08078 | alt.negative.9:117568766, | | | | | | | | | | |
| 0.01521 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0089 | alt.negative.11:128443099, | | | | | | | | | | |
| −0.01676 | ref.negative.11:128443099, | | | | | | | | | | |
| 0 | ref.recessive.11:128443099, | | | | | | | | | | |
| 0 | haploinsuff11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.45098039 | 0.45769231 | 0.4375 | 0.45714286 | 0.4375 | 0.26923077 | 0.64 | ileum.bma.model.clus- |
| −0.01455 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05175 | ref.recessive.21:45658474, | | | | | | | | | | |
| 0 | alt.negative.21:45658474, | | | | | | | | | | |
| −0.01642 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06861 | ref.negative.16:11331509, | | | | | | | | | | |
| 0 | alt.recessive.16:11331509, | | | | | | | | | | |
| −0.02348 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00327 | alt.negative.16:6147645, | | | | | | | | | | |
| −0.01508 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00209 | alt.negative.11:128443099, | | | | | | | | | | |
| −0.03004 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.45098039 | 0.46923077 | 0.4375 | 0.45714286 | 0.4375 | 0.26923077 | 0.64 | ileum.bma.model.clus- |
| −0.02233 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05181 | ref.negative.16:11331509, | | | | | | | | | | |
| 0 | alt.recessive.16:11331509, | | | | | | | | | | |
| −0.01295 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07358 | alt.negative.9:117568766, | | | | | | | | | | |
| 0.02039 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00977 | alt.negative.16:6147645, | | | | | | | | | | |
| −0.00774 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.0185 | alt.negative.11:128443099, | | | | | | | | | | |
| −0.00294 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.45098039 | 0.43846154 | 0.4375 | 0.45714286 | 0.4375 | 0.26923077 | 0.64 | ileum.bma.model.clus- |
| 0.05042 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05085 | ref.recessive.21:45658474, | | | | | | | | | | |
| 0 | alt.negative.21:45658474, | | | | | | | | | | |
| −0.02233 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05171 | ref.negative.20:62305274, | | | | | | | | | | |
| 0 | alt.recessive.20:62305274, | | | | | | | | | | |
| 0.02061 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0769 | ref.negative.16:11331509, | | | | | | | | | | |
| 0 | alt.recessive.16:11331509, | | | | | | | | | | |
| −0.02204 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0705 | alt.negative.9:117568766, | | | | | | | | | | |
| 0.00618 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.5:11562221, | 5 | 0.31372549 | 0.45098039 | 0.42769231 | 0.4375 | 0.45714286 | 0.4375 | 0.26923077 | 0.64 | ileum.bma.model.clus- |
| 0.04295 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05105 | ref.recessive.21:45658474, | | | | | | | | | | |
| 0 | alt.negative.21:45658474, | | | | | | | | | | |
| −0.01255 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.03725 | ref.negative.20:62305274, | | | | | | | | | | |
| 0 | alt.recessive.20:62305274, | | | | | | | | | | |
| 0.0205 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00592 | alt.negative.16:6147645, | | | | | | | | | | |
| −0.01813 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00494 | alt.negative.11:128443099, | | | | | | | | | | |
| −0.01135 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.45098039 | 0.44384615 | 0.4375 | 0.45714286 | 0.4375 | 0.26923077 | 0.64 | ileum.bma.model.clus- |
| 0.04687 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.0623 | ref.negative.20:62305274, | | | | | | | | | | |
| 0 | alt.recessive.20:62305274, | | | | | | | | | | |
| 0.01489 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07748 | ref.negative.16:11331509, | | | | | | | | | | |
| 0 | alt.recessive.16:11331509, | | | | | | | | | | |
| −0.02143 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.09542 | alt.negative.9:117568766, | | | | | | | | | | |
| 0.02422 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01387 | alt.negative.11:128443099, | | | | | | | | | | |
| −0.01145 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.45098039 | 0.45769231 | 0.4375 | 0.45714286 | 0.4375 | 0.26923077 | 0.64 | ileum.bma.model.clus- |
| 0.05822 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07041 | ref.negative.16:11331509, | | | | | | | | | | |
| 0 | alt.recessive.16:11331509, | | | | | | | | | | |
| −0.03057 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0947 | alt.negative.9:117568766, | | | | | | | | | | |
| 0.01784 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.04274 | alt.negative.16:6147645, | | | | | | | | | | |
| −0.01645 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02806 | alt.negative.11:128443099, | | | | | | | | | | |
| −0.00622 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.03529 | ref.recessive.21:45658474, | 5 | 0.31372549 | 0.45098039 | 0.43692308 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.03222 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.reccssive.21:45658474, | | | | | | | | | | |
| −0.05864 | ref.negative.20:62305274, | | | | | | | | | | |
| 0 | alt.recessive.20:62305274, | | | | | | | | | | |
| 0.02357 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06437 | ref.negative.16:11331509, | | | | | | | | | | |
| 0 | alt.recessive.16:11331509, | | | | | | | | | | |
| −0.01385 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08327 | alt.negative.9:117568766, | | | | | | | | | | |
| 0.01261 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01217 | alt.negative.16:6147645, | | | | | | | | | | |
| −0.02206 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.0725 | ref.recessive.21:45658474, | 5 | 0.31372549 | 0.45098039 | 0.42076923 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| 0.00509 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06174 | ref.negative.20:62305274, | | | | | | | | | | |
| 0 | alt.recessive.20:62305274, | | | | | | | | | | |
| 0.0191 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07269 | ref.negative.16:11331509, | | | | | | | | | | |
| 0 | alt.recessive.16:11331509, | | | | | | | | | | |
| −0.02432 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06817 | alt.negative.9:117568766, | | | | | | | | | | |
| 0.0082 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| −0.00144 | alt.negative.11:128443099, | | | | | | | | | | |
| −0.02673 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.06433 | ref.recessive.21:45658474, | 5 | 0.31372549 | 0.45098039 | 0.42692308 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.01066 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06033 | ref.negative.20:62305274, | | | | | | | | | | |
| 0 | alt.recessive.20:62305274, | | | | | | | | | | |
| 0.03111 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06427 | ref.negative.16:11331509, | | | | | | | | | | |
| 0 | alt.recessive.16:11331509, | | | | | | | | | | |
| −0.00165 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.02765 | alt.negative.16:6147645, | | | | | | | | | | |
| −0.00701 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.01193 | alt.negative.11:128443099, | | | | | | | | | | |
| −0.02983 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.0716 | ref.recessive.21:45658474, | 5 | 0.31372549 | 0.45098039 | 0.44846154 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.0026 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0635 | ref.negative.16:11331509, | | | | | | | | | | |
| 0 | alt.recessive.16:11331509, | | | | | | | | | | |
| −0.022 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06586 | alt.negative.9:117568766, | | | | | | | | | | |
| 0.00851 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00111 | alt.negative.16:6147645, | | | | | | | | | | |
| −0.01176 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01241 | alt.negative.11:128443099, | | | | | | | | | | |
| −0.02098 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.33333333 | 0.43137255 | 0.41076923 | 0.41176471 | 0.44117647 | 0.41176471 | 0.26923077 | 0.6 | ileum.bma.model.clus- |
| −0.02454 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03714 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05281 | ref.negative.20:62305274, | | | | | | | | | | |
| 0 | alt.recessive.20:62305274, | | | | | | | | | | |
| 0.00207 | ref.recessive.20:62305274, | | | | | | | | | | |
| 0 | alt.negative.20:62305274, | | | | | | | | | | |
| 0 | haploinsuff.20;62305274, | | | | | | | | | | |
| 0 | additive.20:62305274, | | | | | | | | | | |
| 0.02287 | alt.negative.16:6147645, | | | | | | | | | | |
| −0.01786 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01741 | alt.negative.11:128443099, | | | | | | | | | | |
| −0.00767 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.33333333 | 0.43137255 | 0.43384615 | 0.41176471 | 0.44117647 | 0.41176471 | 0.26923077 | 0.6 | ileum.bma.model.clus- |
| 0.03254 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04333 | ref.recessive.21:45658474, | | | | | | | | | | |
| 0 | alt.negative.21:45658474, | | | | | | | | | | |
| −0.0231 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05469 | ref.negative.20:62305274, | | | | | | | | | | |
| 0 | alt.recessive.20:62305274, | | | | | | | | | | |
| 0.02495 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08736 | alt.negative.9:117568766, | | | | | | | | | | |
| 0.02051 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02087 | alt.negative.16:6147645, | | | | | | | | | | |
| −0.02589 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 5 | 0.33333333 | 0.43137255 | 0.45461538 | 0.41176471 | 0.44117647 | 0.41176471 | 0.26923077 | 0.6 | ileum.bma.model.clus- |
| 0.05444 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04311 | ref.recessive.21:45658474, | | | | | | | | | | |
| 0 | alt.negative.21:45658474, | | | | | | | | | | |
| −0.01764 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0676 | ref.negative.16:11331509, | | | | | | | | | | |
| 0 | alt.recessive.16:11331509, | | | | | | | | | | |
| −0.02983 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08911 | alt.negative.9:117568766, | | | | | | | | | | |
| 0.01422 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02435 | alt.negative.11:128443099, | | | | | | | | | | |
| −0.01988 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.33333333 | 0.43137255 | 0.44769231 | 0.41176471 | 0.44117647 | 0.41176471 | 0.26923077 | 0.6 | ileum.bma.model.clus- |
| 0.03599 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05814 | ref.recessive.21:45658474, | | | | | | | | | | |
| 0 | alt.negative.21:45658474, | | | | | | | | | | |
| −0.00867 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06406 | ref.negative.16:11331509, | | | | | | | | | | |
| 0 | alt.recessive.16:11331509, | | | | | | | | | | |
| −0.02415 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01061 | alt.negative.16:6147645, | | | | | | | | | | |
| −0.01645 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00208 | alt.negative.11:128443099, | | | | | | | | | | |
| −0.01929 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.35294118 | 0.41176471 | 0.41384615 | 0.38888889 | 0.42424242 | 0.38888889 | 0.26923077 | 0.56 | ileum.bma.model.clus- |
| −0.01234 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03305 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06327 | ref.recessive.21:45658474, | | | | | | | | | | |
| 0 | alt.negative.21:45658474, | | | | | | | | | | |
| −0.0028 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05301 | ref.negative.20:62305274, | | | | | | | | | | |
| 0 | alt.recessive.20:62305274, | | | | | | | | | | |
| 0.01141 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00281 | alt.negative.16:6147645, | | | | | | | | | | |
| −0.0131 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.35294118 | 0.41176471 | 0.43461538 | 0.38888889 | 0.42424242 | 0.38888889 | 0.26923077 | 0.56 | ileum.bma.model.clus- |
| −0.00749 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04883 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05951 | ref.negative.20:62305274, | | | | | | | | | | |
| 0 | alt.recessive.20:62305274, | | | | | | | | | | |
| 0.02149 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08872 | alt.negative.9:117568766, | | | | | | | | | | |
| 0.02399 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02378 | alt.negative.11:128443099, | | | | | | | | | | |
| −0.00943 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.35294118 | 0.41176471 | 0.43461538 | 0.38888889 | 0.42424242 | 0.38888889 | 0.26923077 | 0.56 | ileum.bma.model.clus- |
| −0.01909 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05882 | ref.recessive.21:45658474, | | | | | | | | | | |
| 0 | alt.negative.21:45658474, | | | | | | | | | | |
| −0.01241 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06038 | ref.negative.20:62305274, | | | | | | | | | | |
| 0 | alt.recessive.20:62305274, | | | | | | | | | | |
| 0.0192 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.00382 | alt.negative.16:6147645, | | | | | | | | | | |
| −0.01177 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00036 | alt.negative.11:128443099, | | | | | | | | | | |
| −0.01555 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.35294118 | 0.41176471 | 0.42692308 | 0.38888889 | 0.42424242 | 0.38888889 | 0.26923077 | 0.56 | ileum.bma.model.clus- |
| 0.04733 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06861 | ref.recessive.21:45658474, | | | | | | | | | | |
| 0 | alt.negative.21:45658474, | | | | | | | | | | |
| 0.00503 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05607 | ref.negative.20:62305274, | | | | | | | | | | |
| 0 | alt.recessive.20:62305274, | | | | | | | | | | |
| 0.01508 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08495 | alt.negative.9:117568766, | | | | | | | | | | |
| 0.0215 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01551 | alt.negative.11:128443099, | | | | | | | | | | |
| −0.02275 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.41176471 | 0.45384615 | 0.375 | 0.42857143 | 0.375 | 0.23076923 | 0.6 | ileum.bma.model.clus- |
| −0.01219 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03759 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05496 | ref.recessive.21:45658474, | | | | | | | | | | |
| 0 | alt.negative.21:45658474, | | | | | | | | | | |
| −0.00152 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04795 | ref.negative.20:62305274, | | | | | | | | | | |
| 0 | alt.recessive.20:62305274, | | | | | | | | | | |
| 0.00721 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01284 | alt.negative.11:128443099, | | | | | | | | | | |
| −0.0202 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.41176471 | 0.48076923 | 0.375 | 0.42857143 | 0.375 | 0.23076923 | 0.6 | ileum.bma.model.clus- |
| −0.01839 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.05167 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0616 | ref.recessive.21:45658474, | | | | | | | | | | |
| 0 | alt.negative.21:45658474, | | | | | | | | | | |
| −0.00558 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.09009 | alt.negative.9:117568766, | | | | | | | | | | |
| 0.01831 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0221 | alt.negative.11:128443099, | | | | | | | | | | |
| −0.01821 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.41176471 | 0.45 | 0.375 | 0.42857143 | 0.375 | 0.23076923 | 0.6 | ileum.bma.model.clus- |
| −0.02383 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05452 | ref.recessive.21:45658474, | | | | | | | | | | |
| 0 | alt.negative.21:45658474, | | | | | | | | | | |
| −0.00511 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06369 | alt.negative.9:117568766, | | | | | | | | | | |
| 0.017 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0049 | alt.negative.16:6147645, | | | | | | | | | | |
| −0.02033 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00659 | alt.negative.11:128443099, | | | | | | | | | | |
| −0.01501 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.41176471 | 0.40692308 | 0.375 | 0.42857143 | 0.375 | 0.23076923 | 0.6 | ileum.bma.model.clus- |
| −0.01639 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.09677 | ref.negative.20:62305274, | | | | | | | | | | |
| 0 | alt.recessive.20:62305274, | | | | | | | | | | |
| 0.02039 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05795 | ref.negative.16:11331509, | | | | | | | | | | |
| 0 | alt.recessive.16:11331509, | | | | | | | | | | |
| −0.00103 | alt.negalive.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07741 | alt.negative.9:117568766, | | | | | | | | | | |
| 0.02351 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02698 | alt.negative.11:128443099, | | | | | | | | | | |
| 0.0041 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.41176471 | 0.46538462 | 0.375 | 0.42857143 | 0.375 | 0.23076923 | 0.6 | ileum.bma.model.clus- |
| 0.05017 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04798 | ref.recessive.21:45658474, | | | | | | | | | | |
| 0 | alt.negative.21:45658474, | | | | | | | | | | |
| −0.01398 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.08475 | ref.negative.16:11331509, | | | | | | | | | | |
| 0 | alt.recessive.16:11331509, | | | | | | | | | | |
| −0.02341 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0719 | alt.negative.9:117568766, | | | | | | | | | | |
| 0.00857 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01043 | alt.negative.16:6147645, | | | | | | | | | | |
| −0.00793 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 5 | 0.31372549 | 0.41176471 | 0.38384615 | 0.375 | 0.42857143 | 0.375 | 0.23076923 | 0.6 | ileum.bma.model.clus- |
| 0.03442 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07012 | ref.negative.20:62305274, | | | | | | | | | | |
| 0 | alt.recessive.20:62305274, | | | | | | | | | | |
| 0.02962 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0669 | ref.negative.16:11331509, | | | | | | | | | | |
| 0 | alt.recessive.16:11331509, | | | | | | | | | | |
| −0.00502 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07845 | alt.negative.9:117568766, | | | | | | | | | | |
| 0.02196 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01294 | alt.negative.16:6147645, | | | | | | | | | | |
| −0.02292 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 5 | 0.31372549 | 0.41176471 | 0.37230769 | 0.375 | 0.42857143 | 0.375 | 0.23076923 | 0.6 | ileum.bma.model.clus- |
| 0.04564 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05989 | ref.negative.20:62305274, | | | | | | | | | | |
| 0 | alt.recessive.20:62305274, | | | | | | | | | | |
| 0.01795 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0655 | ref.negative.16:11331509, | | | | | | | | | | |
| 0 | alt.recessive.16:11331509, | | | | | | | | | | |
| −0.01487 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01803 | alt.negative.16:6147645, | | | | | | | | | | |
| −0.02142 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01381 | alt.negative.11:128443099, | | | | | | | | | | |
| −0.0138 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.05044 | ref.recessive.21:45658474, | 5 | 0.31372549 | 0.41176471 | 0.42538462 | 0.375 | 0.42857143 | 0.375 | 0.23076923 | 0.6 | ileum.bma.model.clus- |
| 0 | alt.negative.21:45658474, | | | | | | | | | | ter.Entero_Clonocyte.RDS |
| −0.0129 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06684 | ref.negative.20:62305274, | | | | | | | | | | |
| 0 | alt.recessive.20:62305274, | | | | | | | | | | |
| 0.01652 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07301 | alt.negative.9:117568766, | | | | | | | | | | |
| 0.01256 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01273 | alt.negative.16:6147645, | | | | | | | | | | |
| 0.0211 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01914 | alt.negative.11:128443099, | | | | | | | | | | |
| −0.01917 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.37254902 | 0.39215686 | 0.44230769 | 0.36842105 | 0.40625 | 0.36842105 | 0.26923077 | 0.52 | ileum.bma.model.clus- |
| −0.0218 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.04864 | ref.recessive.21:45658474, | | | | | | | | | | |
| 0 | alt.negative.21:45658474, | | | | | | | | | | |
| −0.00968 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05837 | ref.negative.20:62305274, | | | | | | | | | | |
| 0 | alt.recessive.20:62305274, | | | | | | | | | | |
| 0.01099 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07098 | alt.negative.9:117568766, | | | | | | | | | | |
| 0.01215 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01916 | alt.negative.11:128443099, | | | | | | | | | | |
| −0.01199 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.33333333 | 0.39215686 | 0.35615385 | 0.35294118 | 0.41176471 | 0.35294118 | 0.23076923 | 0.56 | ileum.bma.model.clus- |
| −0.01726 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0306 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06951 | ref.negative.20:62305274, | | | | | | | | | | |
| 0 | alt.recessive.20:62305274, | | | | | | | | | | |
| 0.02452 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06374 | ref.negative.16:11331509, | | | | | | | | | | |
| 0 | alt.recessive.16:11331509, | | | | | | | | | | |
| −0.00695 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00949 | alt.negative.16:6147645, | | | | | | | | | | |
| −0.01513 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 5 | 0.33333333 | 0.39215686 | 0.38384615 | 0.35294118 | 0.41176471 | 0.35294118 | 0.23076923 | 0.56 | ileum.bma.model.clus- |
| −0.02675 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03303 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07924 | ref.negative.20:62305274, | | | | | | | | | | |
| 0 | alt.recessive.20:62305274, | | | | | | | | | | |
| 0.00972 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06097 | ref.negative.16:11331509, | | | | | | | | | | |
| 0 | alt.recessive.16:11331509, | | | | | | | | | | |
| −0.0171 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01829 | alt.negative.11:128443099, | | | | | | | | | | |
| −0.0114 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.33333333 | 0.39215686 | 0.37538462 | 0.35294118 | 0.41176471 | 0.35294118 | 0.23076923 | 0.56 | ileum.bma.model.clus- |
| −0.01442 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.09317 | ref.negative.20:62305274, | | | | | | | | | | |
| 0 | alt.recessive.20:62305274, | | | | | | | | | | |
| 0.02068 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07525 | ref.negative.16:11331509, | | | | | | | | | | |
| 0 | alt.recessive.16:11331509, | | | | | | | | | | |
| −0.01006 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0777 | alt.negative.9:117568766, | | | | | | | | | | |
| 0.02095 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01127 | alt.negative.16:6147645, | | | | | | | | | | |
| −0.01316 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.08062 | ref.negative.20:62305274, | 5 | 0.33333333 | 0.39215686 | 0.38461538 | 0.35294118 | 0.41176471 | 0.35294118 | 0.23076923 | 0.56 | ileum.bma.model.clus- |
| 0 | alt.recessive.20:62305274, | | | | | | | | | | ter.Entero_Clonocyte.RDS |
| 0.01564 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06392 | ref.negative.16:11331509, | | | | | | | | | | |
| 0 | alt.recessive.16:11331509, | | | | | | | | | | |
| −0.0063 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07636 | alt.negative.9:117568766, | | | | | | | | | | |
| 0.02549 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00425 | alt.negative.16:6147645, | | | | | | | | | | |
| −0.01521 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00988 | alt.negative.11:128443099, | | | | | | | | | | |
| −0.00793 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.41176471 | 0.35294118 | 0.38692308 | 0.33333333 | 0.36666667 | 0.33333333 | 0.26923077 | 0.44 | ileum.bma.model.clus- |
| 0.04286 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05326 | ref.negative.20:62305274, | | | | | | | | | | |
| 0 | alt.recessive.20:62305274, | | | | | | | | | | |
| 0.0184 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07749 | alt.negative.9:117568766, | | | | | | | | | | |
| 0.01766 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01956 | alt.negative.16:6147645, | | | | | | | | | | |
| −0.01829 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01857 | alt.negative.11:128443099, | | | | | | | | | | |
| −0.00632 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.37254902 | 0.46615385 | 0.3125 | 0.4 | 0.3125 | 0.19230769 | 0.56 | ileum.bma.model.clus- |
| −0.02041 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03096 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04548 | ref.recessive.21:45658474, | | | | | | | | | | |
| 0 | alt.negative.21:45658474, | | | | | | | | | | |
| −0.01623 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.00275 | alt.negative.16:6147645, | | | | | | | | | | |
| −0.00786 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01109 | alt.negative.11:128443099, | | | | | | | | | | |
| −0.01362 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.37254902 | 0.43153846 | 0.3125 | 0.4 | 0.3125 | 0.19230769 | 0.56 | ileum.bma.model.clus- |
| 0.0397 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03168 | ref.recessive.21:45658474, | | | | | | | | | | |
| 0 | alt.negative.21:45658474, | | | | | | | | | | |
| −0.03053 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04716 | ref.negative.20:62305274, | | | | | | | | | | |
| 0 | alt.recessive.20:62305274, | | | | | | | | | | |
| 0.02475 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0695 | ref.negative.16:11331509, | | | | | | | | | | |
| 0 | alt.recessive.16:11331509, | | | | | | | | | | |
| −0.02208 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.00065 | alt.negative.11:128443099, | | | | | | | | | | |
| −0.02696 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 5 | 0.31372549 | 0.37254902 | 0.44 | 0.3125 | 0.4 | 0.3125 | 0.19230769 | 0.56 | ileum.bma.model.clus- |
| 0.04938 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06299 | ref.recessive.21:45658474, | | | | | | | | | | |
| 0 | alt.negative.21:45658474, | | | | | | | | | | |
| 0.00136 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.08208 | alt.negative.9:117568766, | | | | | | | | | | |
| 0.02018 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0143 | alt.negative.16:6147645, | | | | | | | | | | |
| −0.02088 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02483 | alt.negative.11:128443099, | | | | | | | | | | |
| −0.01952 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 5 | 0.31372549 | 0.33333333 | 0.36769231 | 0.25 | 0.37142857 | 0.25 | 0.15384615 | 0.52 | ileum.bma.model.clus- |
| −0.02894 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.09543 | ref.negative.20:62305274, | | | | | | | | | | |
| 0 | alt.recessive.20:62305274, | | | | | | | | | | |
| 0.01537 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05606 | ref.negative.16:11331509, | | | | | | | | | | |
| 0 | alt.recessive.16:11331509, | | | | | | | | | | |
| 0 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00752 | alt.negative.16:6147645, | | | | | | | | | | |
| −0.0148 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01411 | alt.negative.11:128443099, | | | | | | | | | | |
| −0.00163 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00471 | alt.recessive.14:98734567, | 4 | 0.39215686 | 0.56862745 | 0.57384615 | 0.6 | 0.5483871 | 0.6 | 0.46153846 | 0.68 | ileum.bma.model.clus- |
| 0 | ref.negative.14:98734567, | | | | | | | | | | ter.Entero_Clonocyte.RDS |
| −0.00491 | alt.recessive.21:45658474, | | | | | | | | | | |
| −9e−04 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| 0.00117 | additive.9:117568766, | | | | | | | | | | |
| 0.00042 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| −2e−05 | alt.negative.16:6147645 | | | | | | | | | | |
| 0 | ref.negative.14:98734567, | 4 | 0.33333333 | 0.54901961 | 0.46230769 | 0.58823529 | 0.52941176 | 0.58823529 | 0.38461538 | 0.72 | ileum.bma.model.clus- |
| −0.00931 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.06821 | alt.negative.9:117568766, | | | | | | | | | | |
| 0.02289 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00882 | alt.negative.16:6147645, | | | | | | | | | | |
| −0.01274 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01965 | alt.negative.11:128443099, | | | | | | | | | | |
| 0.00211 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00477 | alt.recessive.14:98734567, | 4 | 0.37254902 | 0.54901961 | 0.56615385 | 0.57894737 | 0.53125 | 0.57894737 | 0.42307692 | 0.68 | ileum.bma.model.clus- |
| 0 | ref.negative.14:98734567, | | | | | | | | | | ter.Entero_Clonocyte.RDS |
| −0.00501 | alt.recessive.21:45658474, | | | | | | | | | | |
| −0.00093 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00461 | alt.recessive.5:11562221, | | | | | | | | | | |
| −0.00018 | additive.5:11562221, | | | | | | | | | | |
| 0 | haploinsuf1.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00101 | additive.9:117568766, | | | | | | | | | | |
| 0.00081 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766 | | | | | | | | | | |
| 0 | ref.negative.14:98734567, | 4 | 0.31372549 | 0.52941176 | 0.45692308 | 0.5625 | 0.51428571 | 0.5625 | 0.34615385 | 0.72 | ileum.bma.model.clus- |
| −0.00948 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04509 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.08063 | alt.negative.9:117568766, | | | | | | | | | | |
| 0.02135 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0224 | alt.negative.16:6147645, | | | | | | | | | | |
| −0.01959 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.31372549 | 0.52941176 | 0.49538462 | 0.5625 | 0.51428571 | 0.5625 | 0.34615385 | 0.72 | ileum.bma.model.clus- |
| 0.05465 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05974 | ref.negative.16:11331509, | | | | | | | | | | |
| 0 | alt.recessive.16:11331509, | | | | | | | | | | |
| −0.02278 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08876 | alt.negative.9:117568766, | | | | | | | | | | |
| 0.0235 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02366 | alt.negative.11:128443099, | | | | | | | | | | |
| −0.00854 | ref.negative.11:128443099, | | | | | | | | | | |
| 0 | ref.recessive.l1:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.39215686 | 0.52941176 | 0.51692308 | 0.55 | 0.51612903 | 0.55 | 0.42307692 | 0.64 | ileum.bma.model.clus- |
| −0.01408 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04761 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.08527 | alt.negative.9:117568766, | | | | | | | | | | |
| 0.02476 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0252 | alt.negative.11:128443099, | | | | | | | | | | |
| −0.00733 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.33333333 | 0.50980392 | 0.48923077 | 0.52941176 | 0.5 | 0.52941176 | 0.34615385 | 0.68 | ileum.bma.model.clus- |
| −0.00628 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04492 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.0671 | ref.negative.16:11331509, | | | | | | | | | | |
| 0 | alt.recessive.16:11331509, | | | | | | | | | | |
| −0.02071 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07719 | alt.negative.9:117568766, | | | | | | | | | | |
| 0.02422 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.33333333 | 0.50980392 | 0.51692308 | 0.52941176 | 0.5 | 0.52941176 | 0.34615385 | 0.68 | ileum.bma.model.clus- |
| −0.01532 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05847 | ref.negative.16:11331509, | | | | | | | | | | |
| 0 | alt.recessive.16:11331509, | | | | | | | | | | |
| −0.01685 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.05998 | alt.negative.9:117568766, | | | | | | | | | | |
| 0.01817 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02161 | alt.negative.11:128443099, | | | | | | | | | | |
| −0.00086 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.33333333 | 0.50980392 | 0.46923077 | 0.52941176 | 0.5 | 0.52941176 | 0.34615385 | 0.68 | ileum.bma.model.clus- |
| 0.05025 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.0705 | ref.negative.16:11331509, | | | | | | | | | | |
| 0 | alt.recessive.16:11331509, | | | | | | | | | | |
| −0.02107 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07676 | alt.negative.9:117568766, | | | | | | | | | | |
| 0.01664 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02352 | alt.negative.16:6147645, | | | | | | | | | | |
| −0.01344 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 4 | 0.37254902 | 0.50980392 | 0.47461538 | 0.52631579 | 0.5 | 0.52631579 | 0.38461538 | 0.64 | ileum.bma.model.clus- |
| −0.01288 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.0608 | ref.recessive.21:45658474, | | | | | | | | | | |
| 0 | alt.negative.21:45658474, | | | | | | | | | | |
| −0.0044 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06717 | ref.negative.16:11331509, | | | | | | | | | | |
| 0 | alt.recessive.16:11331509, | | | | | | | | | | |
| −0.02111 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06968 | alt.negative.9:117568766, | | | | | | | | | | |
| 0.00921 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.39215686 | 0.49019608 | 0.47538462 | 0.5 | 0.48387097 | 0.5 | 0.38461538 | 0.6 | ileum.bma.model.clus- |
| −0.0113 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04766 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04697 | ref.recessive.21:45658474, | | | | | | | | | | |
| 0 | alt.negative.21:45658474, | | | | | | | | | | |
| −0.01657 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05978 | ref.negative.16:11331509, | | | | | | | | | | |
| 0 | alt.recessive.16:11331509, | | | | | | | | | | |
| −0.02056 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 4 | 0.31372549 | 0.49019608 | 0.45923077 | 0.5 | 0.48571429 | 0.5 | 0.30769231 | 0.68 | ileum.bma.model.clus- |
| −0.01348 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03484 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.01324 | alt.negative.16:6147645, | | | | | | | | | | |
| −0.01329 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02442 | alt.negative.11:128443099, | | | | | | | | | | |
| −0.00116 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.35294118 | 0.49019608 | 0.43461538 | 0.5 | 0.48484848 | 0.5 | 0.34615385 | 0.64 | ileum.bma.model.clus- |
| −0.01838 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.0519 | ref.recessive.21:45658474, | | | | | | | | | | |
| 0 | alt.negative.21:45658474, | | | | | | | | | | |
| −0.01241 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06627 | ref.negative.20:62305274, | | | | | | | | | | |
| 0 | alt.recessive.20:62305274, | | | | | | | | | | |
| 0.01392 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05531 | ref.negative.16:11331509, | | | | | | | | | | |
| 0 | alt.recessive.16:11331509, | | | | | | | | | | |
| −0.01249 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.5:11562221, | 4 | 0.31372549 | 0.49019608 | 0.45384615 | 0.5 | 0.48571429 | 0.5 | 0.30769231 | 0.68 | ileum.bma.model.clus- |
| 0.04692 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03714 | ref.recessive.21:45658474, | | | | | | | | | | |
| 0 | alt.negative.21:45658474, | | | | | | | | | | |
| −0.02796 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04143 | ref.negative.20:62305274, | | | | | | | | | | |
| 0 | alt.recessive.20:62305274, | | | | | | | | | | |
| 0.01964 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08717 | alt.negative.9:117568766, | | | | | | | | | | |
| 0.0161 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.5:11562221, | 4 | 0.39215686 | 0.49019608 | 0.44 | 0.5 | 0.48387097 | 0.5 | 0.38461538 | 0.6 | ileum.bma.model.clus- |
| 0.0508 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06346 | ref.recessive.21:45658474, | | | | | | | | | | |
| 0 | alt.negative.21:45658474, | | | | | | | | | | |
| 0.00402 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06709 | alt.negative.9:117568766, | | | | | | | | | | |
| 0.01428 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0125 | alt.negative.16:6147645, | | | | | | | | | | |
| −0.02038 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.06252 | ref.recessive.21:45658474, | 4 | 0.35294118 | 0.49019608 | 0.42076923 | 0.5 | 0.48484848 | 0.5 | 0.34615385 | 0.64 | ileum.bma.model.clus- |
| 0 | alt.negative.21:45658474, | | | | | | | | | | ter.Entero_Clonocyte.RDS |
| −0.01194 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07004 | ref.negative.20:62305274, | | | | | | | | | | |
| 0 | alt.recessive.20:62305274, | | | | | | | | | | |
| 0.02843 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07686 | alt.negative.9:117568766, | | | | | | | | | | |
| 0.01156 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.00519 | alt.negative.11:128443099, | | | | | | | | | | |
| −0.01303 | ref.negative11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.04771 | ref.recessive.21:45658474, | 4 | 0.35294118 | 0.49019608 | 0.48076923 | 0.5 | 0.48484848 | 0.5 | 0.34615385 | 0.64 | ileum.bma.model.clus- |
| 0 | alt.negative.21:45658474, | | | | | | | | | | ter.Entero_Clonocyte.RDS |
| −0.01981 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05084 | ref.negative.16:11331509, | | | | | | | | | | |
| 0 | alt.recessive.16:11331509, | | | | | | | | | | |
| −0.01473 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06746 | alt.negative.9:117568766, | | | | | | | | | | |
| 0.01571 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| −0.00973 | alt.negative.16:6147645, | | | | | | | | | | |
| −0.00679 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.06236 | ref.negative.16:11331509, | 4 | 0.35294118 | 0.49019608 | 0.49153846 | 0.5 | 0.48484848 | 0.5 | 0.34615385 | 0.64 | ileum.bma.model.clus- |
| 0 | alt.recessive.16:11331509, | | | | | | | | | | ter.Entero_Clonocyte.RDS |
| −0.02095 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07108 | alt.negative.9:117568766, | | | | | | | | | | |
| 0.01658 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02344 | alt.negative.16:6147645, | | | | | | | | | | |
| −0.0148 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02861 | alt.negative.11:128443099, | | | | | | | | | | |
| −0.00429 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00507 | alt.recessive.14:98734567, | 1 | 0.31372549 | 0.49019608 | 0.54 | 0.5 | 0.48571429 | 0.5 | 0.30769231 | 0.68 | ileum.bma.model.clus- |
| 0 | ref.negative.14:98734567, | | | | | | | | | | ter.Entero_Clonocyte.RDS |
| −0.00511 | alt.recessive.21:45658474, | | | | | | | | | | |
| −0.00085 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00464 | alt.recessive.5:11562221, | | | | | | | | | | |
| −0.00028 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00039 | alt.negative.16:6147645 | | | | | | | | | | |
| −0.00519 | alt.recessive.21:45658474, | 4 | 0.47058824 | 0.49019608 | 0.52923077 | 0.5 | 0.48148148 | 0.5 | 0.46153846 | 0.52 | ileum.bma.model.clus- |
| −0.00093 | additive.21:45658474, | | | | | | | | | | ter.Entero_Clonocyte.RDS |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.0039 | alt.recessive.5:11562221, | | | | | | | | | | |
| −0.00048 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00087 | additive.9:117568766, | | | | | | | | | | |
| 0.00181 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| −2e−04 | alt.negative.16:6147645 | | | | | | | | | | |
| 0 | ref.negative.5:11562221, | 4 | 0.37254902 | 0.47058824 | 0.43307692 | 0.47368421 | 0.46875 | 0.47368421 | 0.34615385 | 0.6 | ileum.bma.model.clus- |
| 0.04642 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05293 | ref.recessive.21:45658474, | | | | | | | | | | |
| 0 | alt.negative.21:45658474, | | | | | | | | | | |
| −0.01371 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05356 | ref.negative.20:62305274, | | | | | | | | | | |
| 0 | alt.recessive.20:62305274, | | | | | | | | | | |
| 0.01718 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06687 | ref.negative.16:11331509, | | | | | | | | | | |
| 0 | alt.recessive.16:11331509, | | | | | | | | | | |
| −0.0208 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 4 | 0.33333333 | 0.47058824 | 0.43230769 | 0.47058824 | 0.47058824 | 0.47058824 | 0.30769231 | 0.64 | ileum.bma.model.clus- |
| −0.0225 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.04932 | ref.recessive.21:45658474, | | | | | | | | | | |
| 0 | alt.negative.21:45658474, | | | | | | | | | | |
| −0.00884 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05535 | ref.negative.20:62305274, | | | | | | | | | | |
| 0 | alt.recessive.20:62305274, | | | | | | | | | | |
| 0.01479 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05825 | alt.negative.9:117568766, | | | | | | | | | | |
| 0.0106 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.33333333 | 0.47058824 | 0.41230769 | 0.47058824 | 0.47058824 | 0.47058824 | 0.30769231 | 0.64 | ileum.bma.model.clus- |
| −0.01189 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05588 | ref.recessive.21:45658474, | | | | | | | | | | |
| 0 | alt.negative.21:45658474, | | | | | | | | | | |
| −0.00911 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05276 | ref.negative.20:62305274, | | | | | | | | | | |
| 0 | alt.recessive.20:62305274, | | | | | | | | | | |
| 0.017 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.003 | alt.negative.11:128443099, | | | | | | | | | | |
| −0.00307 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive11:128443099, | | | | | | | | | | |
| 0 | alt.recessive.11:128443099 | | | | | | | | | | |
| 0 | ref.negative.14:98734567, | 4 | 0.33333333 | 0.47058824 | 0.45923077 | 0.47058824 | 0.47058824 | 0.47058824 | 0.30769231 | 0.64 | ileum.bma.model.clus- |
| −0.0271 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05826 | ref.recessive.21:45658474, | | | | | | | | | | |
| 0 | alt.negative.21:45658474, | | | | | | | | | | |
| −0.00704 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0519 | ref.negative.16:11331509, | | | | | | | | | | |
| 0 | alt.recessive.16:11331509, | | | | | | | | | | |
| −0.01483 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00069 | alt.negative.16:6147645, | | | | | | | | | | |
| −0.01984 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.33333333 | 0.47058824 | 0.43153846 | 0.47058824 | 0.47058824 | 0.47058824 | 0.30769231 | 0.64 | ileum.bma.model.clus- |
| 0.0456 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.08429 | alt.negative.9:117568766, | | | | | | | | | | |
| 0.02016 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.03996 | alt.negative.16:6147645, | | | | | | | | | | |
| −0.02395 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01666 | alt.negative.11:128443099, | | | | | | | | | | |
| −0.01007 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.03439 | ref.recessive.21:45658474, | 4 | 0.33333333 | 0.47058824 | 0.43538462 | 0.47058824 | 0.47058824 | 0.47058824 | 0.30769231 | 0.64 | ileum.bma.model.clus- |
| 0 | alt.negative.21:45658474, | | | | | | | | | | ter.Entero_Clonocyte.RDS |
| −0.03299 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06546 | ref.negative.20:62305274, | | | | | | | | | | |
| 0 | alt.recessive.20:62305274, | | | | | | | | | | |
| 0.02934 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07273 | ref.negative.16:11331509, | | | | | | | | | | |
| 0 | alt.recessive.16:11331509, | | | | | | | | | | |
| −0.02167 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07195 | alt.negative.9:117568766, | | | | | | | | | | |
| 0.00703 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.00533 | alt.recessive.14:98734567, | 4 | 0.33333333 | 0.47058824 | 0.51846154 | 0.47058824 | 0.47058824 | 0.47058824 | 0.30769231 | 0.64 | ileum.bma.model.clus- |
| 0 | ref.negative.14:98734567, | | | | | | | | | | ter.Entero_Clonocyte.RDS |
| −0.0039 | alt.recessive.5:11562221, | | | | | | | | | | |
| −0.00022 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00064 | additive.9:117568766, | | | | | | | | | | |
| 0.00189 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| 0.00024 | alt.negative.16:6147645 | | | | | | | | | | |
| 0 | ref.negative.5:11562221, | 4 | 0.43137255 | 0.45098039 | 0.41923077 | 0.45454545 | 0.44827586 | 0.45454545 | 0.38461538 | 0.52 | ileum.bma.model.clus- |
| 0.0476 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07333 | ref.negative.20:62305274, | | | | | | | | | | |
| 0 | alt.recessive.20:62305274, | | | | | | | | | | |
| 0.02833 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06571 | ref.negative.16:11331509, | | | | | | | | | | |
| 0 | alt.recessive.16:11331509, | | | | | | | | | | |
| −0.01143 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08833 | alt.negative.9:117568766, | | | | | | | | | | |
| 0.01853 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.5:11562221, | 4 | 0.39215686 | 0.45098039 | 0.44769231 | 0.45 | 0.4516129 | 0.45 | 0.34615385 | 0.56 | ileum.bma.model.clus- |
| 0.05102 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06174 | ref.recessive.21:45658474, | | | | | | | | | | |
| 0 | alt.negative.21:45658474, | | | | | | | | | | |
| 7e−04 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.08011 | ref.negative.16:11331509, | | | | | | | | | | |
| 0 | alt.recessive.16:11331509, | | | | | | | | | | |
| −0.02788 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08021 | alt.negative.9:117568766, | | | | | | | | | | |
| 0.00946 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.35294118 | 0.45098039 | 0.45153846 | 0.44444444 | 0.45454545 | 0.44444444 | 0.30769231 | 0.6 | ileum.bma.model.clus- |
| −0.02315 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03235 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03229 | ref.recessive.21:45658474, | | | | | | | | | | |
| 0 | alt.negative.21:45658474, | | | | | | | | | | |
| −0.02957 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.03853 | ref.negative.20:62305274, | | | | | | | | | | |
| 0 | alt.recessive.20:62305274, | | | | | | | | | | |
| 0.00849 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274 | | | | | | | | | | |
| 0 | ref.negative.14:98734567, | 4 | 0.35294118 | 0.45098039 | 0.38846154 | 0.44444444 | 0.45454545 | 0.44444444 | 0.30769231 | 0.6 | ileum.bma.model.clus- |
| −0.02766 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.02959 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07928 | ref.negative.20:62305274, | | | | | | | | | | |
| 0 | alt.recessive.20:62305274, | | | | | | | | | | |
| 0.00751 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01624 | alt.negative.16:6147645, | | | | | | | | | | |
| −0.01572 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.35294118 | 0.45098039 | 0.46 | 0.44444444 | 0,45454545 | 0.44444444 | 0.30769231 | 0.6 | ileum.bma.model.clus- |
| 0.0402 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05598 | ref.negative.16:11331509, | | | | | | | | | | |
| 0 | alt.recessive.16:11331509, | | | | | | | | | | |
| −0.01558 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.0225 | alt.negative.l6:6147645, | | | | | | | | | | |
| −0.01333 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01113 | alt.negative.11:128443099, | | | | | | | | | | |
| 0.00136 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.03667 | ref.recessive.21:45658474, | 4 | 0.35294118 | 0.45098039 | 0.46076923 | 0.44444444 | 0.45454545 | 0.44444444 | 0.30769231 | 0.6 | ileum.bma.model.clus- |
| 0 | alt.negative.21:45658474, | | | | | | | | | | ter.Entero_Clonocyte.RDS |
| −0.02481 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.08218 | alt.negative.9:117568766, | | | | | | | | | | |
| 0.02281 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01028 | alt.negative.16:6147645, | | | | | | | | | | |
| −0.01958 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00796 | alt.negative.11:128443099, | | | | | | | | | | |
| −0.01682 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.07555 | ref.negative.20:62305274, | 4 | 0.35294118 | 0.45098039 | 0.40615385 | 0.44444444 | 0.45454545 | 0.44444444 | 0.30769231 | 0.6 | ileum.bma.model.clus- |
| 0 | alt.recessive.20:62305274, | | | | | | | | | | ter.Entero_Clonocyte.RDS |
| 0.01443 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07707 | alt.negative.9:117568766, | | | | | | | | | | |
| 0.02082 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01799 | alt.negative.16:6147645, | | | | | | | | | | |
| −0.01958 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02573 | alt.negative.11:128443099, | | | | | | | | | | |
| −0.00362 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.49019608 | 0.43137255 | 0.42923077 | 0.44 | 0.42307692 | 0.44 | 0.42307692 | 0.44 | ileum.bma.model.clus- |
| −0.02272 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.0884 | ref.negative.20:62305274, | | | | | | | | | | |
| 0 | alt.recessive.20:62305274, | | | | | | | | | | |
| 0.00931 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0877 | alt.negative.9:117568766, | | | | | | | | | | |
| 0.02895 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02626 | alt.negative.11:128443099, | | | | | | | | | | |
| −0.01162 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive11:128443099, | | | | | | | | | | |
| 0 | alt.recessive.11:128443099 | | | | | | | | | | |
| 0 | ref.negative.14:98734567, | 4 | 0.31372549 | 0.45098039 | 0.44230769 | 0.4375 | 0.45714286 | 0.4375 | 0.26923077 | 0.64 | ileum.bma.model.clus- |
| −0.01204 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0455 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.08234 | ref.recessive.21:45658474, | | | | | | | | | | |
| 0 | alt.negative.21:45658474, | | | | | | | | | | |
| 0.01454 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.0671 | alt.negative.9:117568766, | | | | | | | | | | |
| 0.00973 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.31372549 | 0.45098039 | 0.47615385 | 0.4375 | 0.45714286 | 0.4375 | 0.26923077 | 0.64 | ileum.bma.model.clus- |
| −0.01158 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03358 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04355 | ref.recessive.21:45658474, | | | | | | | | | | |
| 0 | alt.negative.21:45658474, | | | | | | | | | | |
| −0.01958 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.00239 | alt.negative.11:128443099, | | | | | | | | | | |
| −0.01318 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.45098039 | 0.44846154 | 0.4375 | 0.45714286 | 0.4375 | 0.26923077 | 0.64 | ileum.bma.model.clus- |
| −0.01515 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04977 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06642 | ref.negative.16:11331509, | | | | | | | | | | |
| 0 | alt.recessive.16:11331509, | | | | | | | | | | |
| −0.01189 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00721 | alt.negative.16:6147645, | | | | | | | | | | |
| −0.00661 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 4 | 0.31372549 | 0.45098039 | 0.45615385 | 0.4375 | 0.45714286 | 0.4375 | 0.26923077 | 0.64 | ileum.bma.model.clus- |
| −0.02019 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0546 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05643 | ref.negative.16:11331509, | | | | | | | | | | |
| 0 | alt.recessive.16:11331509, | | | | | | | | | | |
| −0.01601 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.02083 | alt.negative.11:128443099, | | | | | | | | | | |
| −0.00085 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.45098039 | 0.46461538 | 0.4375 | 0.45714286 | 0.4375 | 0.26923077 | 0.64 | ileum.bma.model.clus- |
| −0.0181 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.06992 | ref.recessive.21:45658474, | | | | | | | | | | |
| 0 | alt.negative.21:45658474, | | | | | | | | | | |
| 0.00287 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05709 | ref.negative.16:11331509, | | | | | | | | | | |
| 0 | alt.recessive.16:11331509, | | | | | | | | | | |
| −0.02247 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00392 | alt.negative.11:128443099, | | | | | | | | | | |
| −0.0225 | ref.negative11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.45098039 | 0.35 | 0.4375 | 0.45714286 | 0.4375 | 0.26923077 | 0.64 | ileum.bma.model.clus- |
| −0.01496 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.08049 | ref.negative.20:62305274, | | | | | | | | | | |
| 0 | alt.recessive.20:62305274, | | | | | | | | | | |
| 0.01426 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05707 | alt.negative.9:117568766, | | | | | | | | | | |
| 0.01907 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01741 | alt.negative.16:6147645, | | | | | | | | | | |
| −0.01594 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 4 | 0.31372549 | 0.45098039 | 0.44153846 | 0.4375 | 0.45714286 | 0.4375 | 0.26923077 | 0.64 | ileum.bma.model.clus- |
| −0.00697 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05028 | ref.negative.16:11331509, | | | | | | | | | | |
| 0 | alt.recessive.16:11331509, | | | | | | | | | | |
| −0.00504 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06821 | alt.negative.9:117568766, | | | | | | | | | | |
| 0.02071 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02252 | alt.negative.16:6147645, | | | | | | | | | | |
| −0.01613 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.31372549 | 0.45098039 | 0.45692308 | 0.4375 | 0.45714286 | 0.4375 | 0.26923077 | 0.64 | ileum.bma.model.clus- |
| 0.03743 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03188 | ref.recessive.21:45658474, | | | | | | | | | | |
| 0 | alt.negative.21:45658474, | | | | | | | | | | |
| −0.03088 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05672 | ref.negative.16:11331509, | | | | | | | | | | |
| 0 | alt.recessive.16:11331509, | | | | | | | | | | |
| −0.01146 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.02476 | alt.negative.16:6147645, | | | | | | | | | | |
| −0.02411 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.05666 | ref.recessive.21:45658474, | 4 | 0.31372549 | 0.45098039 | 0.45384615 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.01164 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04403 | ref.negative.20:62305274, | | | | | | | | | | |
| 0 | alt.recessive.20:62305274, | | | | | | | | | | |
| 0.01217 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05968 | ref.negative.16:11331509, | | | | | | | | | | |
| 0 | alt.recessive.16:11331509, | | | | | | | | | | |
| −0.02201 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.00951 | alt.negative.11:128443099, | | | | | | | | | | |
| −0.02373 | ref.negative11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.06182 | ref.recessive.21:45658474, | 4 | 0.31372549 | 0.45098039 | 0.45615385 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.00652 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07255 | ref.negative.16:11331509, | | | | | | | | | | |
| 0 | alt.recessive.16:11331509, | | | | | | | | | | |
| −0.02937 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.06496 | alt.negative.9:117568766, | | | | | | | | | | |
| 0.00929 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01433 | alt.negative.11:128443099, | | | | | | | | | | |
| −0.02379 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.41176471 | 0.43137255 | 0.43692308 | 0.42857143 | 0.43333333 | 0.42857143 | 0.34615385 | 0.52 | ileum.bma.model.clus- |
| 0.04064 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03279 | ref.recessive.21:45658474, | | | | | | | | | | |
| 0 | alt.negative.21:45658474, | | | | | | | | | | |
| −0.02613 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.02401 | alt.negative.16:6147645, | | | | | | | | | | |
| −0.02324 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00247 | alt.negative.11:128443099, | | | | | | | | | | |
| −0.02003 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.41176471 | 0.43137255 | 0.40769231 | 0.42857143 | 0.43333333 | 0.42857143 | 0.34615385 | 0.52 | ileum.bma.model.clus- |
| 0.03769 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.0631 | ref.negative.20:62305274, | | | | | | | | | | |
| 0 | alt.recessive.20:62305274, | | | | | | | | | | |
| 0.0153 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06868 | ref.negative.16:11331509, | | | | | | | | | | |
| 0 | alt.recessive.16:11331509, | | | | | | | | | | |
| −0.01164 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01125 | alt.negative.11:128443099, | | | | | | | | | | |
| −0.01268 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.50980392 | 0.41176471 | 0.41846154 | 0.42307692 | 0.4 | 0.42307692 | 0.42307692 | 0.4 | ileum.bma.model.clus- |
| −0.01978 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04397 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05826 | ref.negative.20:62305274, | | | | | | | | | | |
| 0 | alt.recessive.20:62305274, | | | | | | | | | | |
| 0.01475 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06803 | alt.negative.9:117568766, | | | | | | | | | | |
| 0.01631 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 4 | 0.37254902 | 0.43137255 | 0.45615385 | 0.42105263 | 0.4375 | 0.42105263 | 0.30769231 | 0.56 | ileum.bma.model.clus- |
| −0.0183 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.0357 | ref.recessive.21:45658474, | | | | | | | | | | |
| 0 | alt.negative.21:45658474, | | | | | | | | | | |
| −0.02327 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04499 | ref.negative.20:62305274, | | | | | | | | | | |
| 0 | alt.recessive.20:62305274, | | | | | | | | | | |
| 0.01606 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.00394 | alt.negative.16:6147645, | | | | | | | | | | |
| −0.01644 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.33333333 | 0.43137255 | 0.46846154 | 0.41176471 | 0.44117647 | 0.41176471 | 0.26923077 | 0.6 | ileum.bma.model.clus- |
| 0.04726 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.0492 | ref.recessive.21:45658474, | | | | | | | | | | |
| 0 | alt.negative.21:45658474, | | | | | | | | | | |
| −0.01192 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06361 | ref.negative.16:11331509, | | | | | | | | | | |
| 0 | alt.recessive.16:11331509, | | | | | | | | | | |
| −0.02598 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.0107 | alt.negative.11:128443099, | | | | | | | | | | |
| −0.0247 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.33333333 | 0.43137255 | 0.47923077 | 0.41176471 | 0.44117647 | 0.41176471 | 0.26923077 | 0.6 | ileum.bma.model.clus- |
| 0.04746 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05698 | ref.recessive.21:45658474, | | | | | | | | | | |
| 0 | alt.negative.21:45658474, | | | | | | | | | | |
| −0.00606 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.08137 | alt.negative.9:117568766, | | | | | | | | | | |
| 0.01618 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0176 | alt.negative.11:128443099, | | | | | | | | | | |
| −0.01475 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.l1:128443099 | | | | | | | | | | |
| 0 | ref.negative.14:98734567, | 4 | 0.39215686 | 0.41176471 | 0.39461538 | 0.4 | 0.41935484 | 0.4 | 0.30769231 | 0.52 | ileum.bma.model.clus- |
| −0.01421 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.07171 | ref.negative.20:62305274, | | | | | | | | | | |
| 0 | alt.recessive.20:62305274, | | | | | | | | | | |
| 0.025 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05811 | ref.negative.16:11331509, | | | | | | | | | | |
| 0 | alt.recessive.16:11331509, | | | | | | | | | | |
| −0.01283 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07776 | alt.negative.9:117568766, | | | | | | | | | | |
| 0.02087 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.5:11562221, | 4 | 0.35294118 | 0,41176471 | 0.44153846 | 0.38888889 | 0.42424242 | 0.38888889 | 0.26923077 | 0.56 | ileum.bma.model.clus- |
| 0.05053 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06346 | ref.negative.20:62305274, | | | | | | | | | | |
| 0 | alt.recessive.20:62305274, | | | | | | | | | | |
| 0.01899 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.08986 | alt.negative.9:117568766, | | | | | | | | | | |
| 0.02612 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0304 | alt.negative.11:128443099, | | | | | | | | | | |
| −0.00556 | ref.negative.11:128443099, | | | | | | | | | | |
| 0 | ref.recessive.l1:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.41176471 | 0.46538462 | 0.375 | 0.42857143 | 0.375 | 0.23076923 | 0.6 | ileum.bma.model.clus- |
| −0.01672 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0371 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06195 | ref.recessive.21:45658474, | | | | | | | | | | |
| 0 | alt.negative.21:45658474, | | | | | | | | | | |
| −0.00353 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.00879 | alt.negative.16:6147645, | | | | | | | | | | |
| −0.01488 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 4 | 0.31372549 | 0.41176471 | 0.40384615 | 0.375 | 0.42857143 | 0.375 | 0.23076923 | 0.6 | ileum.bma.model.clus- |
| −0.02097 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04034 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05992 | ref.negative.20:62305274, | | | | | | | | | | |
| 0 | alt.recessive.20:62305274, | | | | | | | | | | |
| 0.01548 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01734 | alt.negative.11:128443099, | | | | | | | | | | |
| −0.00349 | ref.negative.11:128443099, | | | | | | | | | | |
| 0 | ref.recessive.11;128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.41176471 | 0.48307692 | 0.375 | 0.42857143 | 0.375 | 0.23076923 | 0.6 | ileum.bma.model.clus- |
| −0.02386 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.03303 | ref.recessive.21:45658474, | | | | | | | | | | |
| 0 | alt.negative.21:45658474, | | | | | | | | | | |
| −0.02344 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06285 | alt.negative.9:117568766, | | | | | | | | | | |
| 0.01499 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01257 | alt.negative.11:128443099, | | | | | | | | | | |
| −0.01095 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.31372549 | 0.41176471 | 0.44461538 | 0.375 | 0.42857143 | 0.375 | 0.23076923 | 0.6 | ileum.bma.model.clus- |
| 0.03325 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.053 | ref.recessive.21:45658474, | | | | | | | | | | |
| 0 | alt.negative.21:45658474, | | | | | | | | | | |
| −0.01203 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06128 | ref.negative.20:62305274, | | | | | | | | | | |
| 0 | alt.recessive.20:62305274, | | | | | | | | | | |
| 0.01093 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00081 | alt.negative.11:128443099, | | | | | | | | | | |
| −0.02004 | ref.negative.11:128443099, | | | | | | | | | | |
| 0 | ref.recessive.l1:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.31372549 | 0.41176471 | 0.37769231 | 0.375 | 0.42857143 | 0.375 | 0.23076923 | 0.6 | ileum.bma.model.clus- |
| 0.03666 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05221 | ref.negative.20:62305274, | | | | | | | | | | |
| 0 | alt.recessive.20:62305274, | | | | | | | | | | |
| 0.02337 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06308 | ref.negative.16:11331509, | | | | | | | | | | |
| 0 | alt.recessive.16:11331509, | | | | | | | | | | |
| −0.01289 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.02575 | alt.negative.16:6147645, | | | | | | | | | | |
| −0.02259 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.31372549 | 0.41176471 | 0.35923077 | 0.375 | 0.42857143 | 0.375 | 0.23076923 | 0.6 | ileum.bma.model.clus- |
| 0.03419 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06695 | ref.negative.20:62305274, | | | | | | | | | | |
| 0 | alt.recessive.20:62305274, | | | | | | | | | | |
| 0.01562 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07961 | alt.negative.9:117568766, | | | | | | | | | | |
| 0.02101 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.035 | alt.negative.16:6147645, | | | | | | | | | | |
| −0.02638 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.01826 | ref.recessive.21:45658474, | 4 | 0.31372549 | 0.41176471 | 0.43923077 | 0.375 | 0.42857143 | 0.375 | 0.23076923 | 0.6 | ileum.bma.model.clus- |
| 0 | alt.negative.21:45658474, | | | | | | | | | | ter.Entero_Clonocyte.RDS |
| −0.03734 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06171 | ref.negative.20:62305274, | | | | | | | | | | |
| 0 | alt.recessive.20:62305274, | | | | | | | | | | |
| 0.02329 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05446 | ref.negative.16:11331509, | | | | | | | | | | |
| 0 | alt.recessive.16:11331509, | | | | | | | | | | |
| −0.00553 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01305 | alt.negative.16:6147645, | | | | | | | | | | |
| −0.02199 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 4 | 0.37254902 | 0.39215686 | 0.39153846 | 0.36842105 | 0.40625 | 0.36842105 | 0.26923077 | 0.52 | ileum.bma.model.clus- |
| −0.01894 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04506 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06884 | ref.negative.20:62305274, | | | | | | | | | | |
| 0 | alt.recessive.20:62305274, | | | | | | | | | | |
| 0.01557 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06058 | ref.negative.16:11331509, | | | | | | | | | | |
| 0 | alt.recessive.16:11331509, | | | | | | | | | | |
| −0.00675 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 4 | 0.37254902 | 0.39215686 | 0.47153846 | 0.36842105 | 0.40625 | 0.36842105 | 0.26923077 | 0.52 | ileum.bma.model.clus- |
| −0.01545 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.03721 | ref.recessive.21:45658474, | | | | | | | | | | |
| 0 | alt.negative.21:45658474, | | | | | | | | | | |
| −0.01944 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.00168 | alt.negative.16:6147645, | | | | | | | | | | |
| −0.01596 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00209 | alt.negative.11:128443099, | | | | | | | | | | |
| −0.01511 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.33333333 | 0.39215686 | 0.43538462 | 0.35294118 | 0.41176471 | 0.35294118 | 0.23076923 | 0.56 | ileum.bma.model.clus- |
| −0.02026 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.0639 | ref.recessive.21:45658474, | | | | | | | | | | |
| 0 | alt.negative.21:45658474, | | | | | | | | | | |
| −0.001 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05062 | alt.negative.9:117568766, | | | | | | | | | | |
| 0.00857 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01788 | alt.negative.16:6147645, | | | | | | | | | | |
| −0.01671 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.04715 | ref.recessive.21:45658474, | 4 | 0.33333333 | 0.39215686 | 0.44692308 | 0.35294118 | 0.41176471 | 0.35294118 | 0.23076923 | 0.56 | ileum.bma.model.clus- |
| 0 | alt.negative.21:45658474, | | | | | | | | | | ter.Entero_Clonocyte.RDS |
| −0.02259 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06135 | ref.negative.20:62305274, | | | | | | | | | | |
| 0 | alt.recessive.20:62305274, | | | | | | | | | | |
| 0.02226 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00295 | alt.negative.16:6147645, | | | | | | | | | | |
| −0.02752 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00026 | alt.negative.11:128443099, | | | | | | | | | | |
| −0.02658 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.10213 | ref.negative.20:62305274, | 4 | 0.33333333 | 0.39215686 | 0.34846154 | 0.35294118 | 0.41176471 | 0.35294118 | 0.23076923 | 0.56 | ileum.bma.model.clus- |
| 0 | alt.recessive.20:62305274, | | | | | | | | | | ter.Entero_Clonocyte.RDS |
| 0.03065 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06598 | ref.negative.16:11331509, | | | | | | | | | | |
| 0 | alt.recessive.16:11331509, | | | | | | | | | | |
| −0.00285 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08385 | alt.negative.9:117568766, | | | | | | | | | | |
| 0.02072 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0264 | alt.negative.16:6147645, | | | | | | | | | | |
| −0.02261 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.06979 | ref.negative.20:62305274, | 4 | 0.33333333 | 0.39215686 | 0.37307692 | 0.35294118 | 0.41176471 | 0.35294118 | 0.23076923 | 0.56 | ileum.bma.model.clus- |
| 0 | alt.recessive.20:62305274, | | | | | | | | | | ter.Entero_Clonocyte.RDS |
| 0.01806 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.066 | ref.negative.16:11331509, | | | | | | | | | | |
| 0 | alt.recessive.16:11331509, | | | | | | | | | | |
| −0.01368 | alt.negative.16:11331509, | | | | | | | | | | |
| 0 | ref.reccssive.16:11331509, | | | | | | | | | | |
| 0 | haploinsuff.16:11331509, | | | | | | | | | | |
| 0 | additive.16:11331509, | | | | | | | | | | |
| 0.01278 | alt.negative.16:6147645, | | | | | | | | | | |
| −0.01997 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01315 | alt.negative.11:128443099, | | | | | | | | | | |
| −0.00572 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.35294118 | 0.37254902 | 0.38615385 | 0.33333333 | 0.39393939 | 0.33333333 | 0.23076923 | 0.52 | ileum.bma.model.clus- |
| −0.02423 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.09534 | ref.negative.20:62305274, | | | | | | | | | | |
| 0 | alt.recessive.20:62305274, | | | | | | | | | | |
| 0.00793 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06589 | ref.negative.16:11331509, | | | | | | | | | | |
| 0 | alt.recessive.16:11331509, | | | | | | | | | | |
| −0.00691 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00991 | alt.negative.11:128443099, | | | | | | | | | | |
| 0.00013 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.05805 | ref.recessive.21:45658474, | 4 | 0.35294118 | 0.37254902 | 0.44769231 | 0.33333333 | 0.39393939 | 0.33333333 | 0.23076923 | 0.52 | ileum.bma.model.clus- |
| 0 | alt.negative.21:45658474, | | | | | | | | | | ter.Entero_Clonocyte.RDS |
| −0.01518 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05206 | ref.negative.16:11331509, | | | | | | | | | | |
| 0 | alt.recessive.16:11331509, | | | | | | | | | | |
| −0.01901 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01005 | alt.negative.16:6147645, | | | | | | | | | | |
| −0.02117 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| −0.00239 | alt.negative.11:128443099, | | | | | | | | | | |
| −0.02419 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.37254902 | 0.39 | 0.3125 | 0.4 | 0.3125 | 0.19230769 | 0.56 | ileum.bma.model.clus- |
| −0.01799 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.06965 | ref.negative.20:62305274, | | | | | | | | | | |
| 0 | alt.recessive.20:62305274, | | | | | | | | | | |
| 0.00826 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.02431 | alt.negative.16:6147645, | | | | | | | | | | |
| −0.02077 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02122 | alt.negative.11:128443099, | | | | | | | | | | |
| 0.00236 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 4 | 0.31372549 | 0.37254902 | 0.43 | 0.3125 | 0.4 | 0.3125 | 0.19230769 | 0.56 | ileum.bma.model.clus- |
| −0.01972 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.04569 | ref.negative.16:11331509, | | | | | | | | | | |
| 0 | alt.recessive.16:11331509, | | | | | | | | | | |
| 0.00012 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01434 | alt.negative.16:6147645, | | | | | | | | | | |
| −0.01281 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01564 | alt.negative.11:128443099, | | | | | | | | | | |
| −0.00279 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 4 | 0.31372549 | 0.37254902 | 0.39615385 | 0.3125 | 0.4 | 0.3125 | 0.19230769 | 0.56 | ileum.bma.model.clus- |
| 0.03082 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06758 | ref.recessive.21:45658474, | | | | | | | | | | |
| 0 | alt.negative.21:45658474, | | | | | | | | | | |
| −0.00229 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04889 | ref.negative.20:62305274, | | | | | | | | | | |
| 0 | alt.recessive.20:62305274, | | | | | | | | | | |
| 0.01645 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.03189 | alt.negative.16:6147645, | | | | | | | | | | |
| −0.03265 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 4 | 0.31372549 | 0.37254902 | 0.37230769 | 0.3125 | 0.4 | 0.3125 | 0.19230769 | 0.56 | ileum.bma.model.clus- |
| 0.03792 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06006 | ref.negative.20:62305274, | | | | | | | | | | |
| 0 | alt.recessive.20:62305274, | | | | | | | | | | |
| 0.01503 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01648 | alt.negative.16:6147645, | | | | | | | | | | |
| −0.01418 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02054 | alt.negative.11:128443099, | | | | | | | | | | |
| −0.00059 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.0595 | ref.recessive.21:45658474, | 4 | 0.31372549 | 0.37254902 | 0.39846154 | 0.3125 | 0.4 | 0.3125 | 0.19230769 | 0.56 | ileum.bma.model.clus- |
| 0 | alt.negative.21:45658474, | | | | | | | | | | ter.Entero_Clonocyte.RDS |
| −0.01342 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05837 | ref.negative.20:62305274, | | | | | | | | | | |
| 0 | alt.recessive.20:62305274, | | | | | | | | | | |
| 0.02518 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07428 | alt.negative.9:117568766, | | | | | | | | | | |
| 0.01562 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive9:117568766, | | | | | | | | | | |
| 0.02447 | alt.negative.16:6147645, | | | | | | | | | | |
| −0.02934 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.08458 | ref.negative.20:62305274, | 4 | 0.31372549 | 0.37254902 | 0.41769231 | 0.3125 | 0.4 | 0.3125 | 0.19230769 | 0.56 | ileum.bma.model.clus- |
| 0 | alt.recessive.20:62305274, | | | | | | | | | | ter.Entero_Clonocyte.RDS |
| 0.01975 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.05943 | ref.negative.16:11331509, | | | | | | | | | | |
| 0 | alt.recessive.16:11331509, | | | | | | | | | | |
| −0.01123 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08836 | alt.negative.9:117568766, | | | | | | | | | | |
| 0.02544 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01555 | alt.negative.11:128443099, | | | | | | | | | | |
| −0.00624 | ref.negative11:128443099, | | | | | | | | | | |
| 0 | ref.recessive.11:128443099, | | | | | | | | | | |
| 0 | haploinsuff.11:128443099, | | | | | | | | | | |
| 0 | additive11:128443099, | | | | | | | | | | |
| 0 | alt.recessive.11:128443099 | | | | | | | | | | |
| 0 | ref.negative.14:98734567, | 4 | 0.33333333 | 0.35294118 | 0.34076923 | 0.29411765 | 0.38235294 | 0.29411765 | 0.19230769 | 0.52 | ileum.bma.model.clus- |
| −0.02666 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.08905 | ref.negative.20:62305274, | | | | | | | | | | |
| 0 | alt.recessive.20:62305274, | | | | | | | | | | |
| 0.02264 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.04556 | ref.negative.16:11331509, | | | | | | | | | | |
| 0 | alt.recessive.16:11331509, | | | | | | | | | | |
| 0.01413 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00825 | alt.negative.16:6147645, | | | | | | | | | | |
| −0.01804 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.00471 | alt.recessive.14:98734567, | 3 | 0.39215686 | 0.56862745 | 0.57769231 | 0.6 | 0.5483871 | 0.6 | 0.46153846 | 0.68 | ileum.bma.model.clus- |
| 0 | ref.negative.14:98734567, | | | | | | | | | | ter.Entero_Clonocyte.RDS |
| −0.00491 | alt.recessive.21:45658474, | | | | | | | | | | |
| −9e−04 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| 0.00117 | additive.9:117568766, | | | | | | | | | | |
| 0.00041 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766 | | | | | | | | | | |
| 0 | ref.negative.14:98734567, | 3 | 0.31372549 | 0.52941176 | 0.5153846 | 0.5625 | 0.51428571 | 0.5625 | 0.34615385 | 0.72 | ileum.bma.model.clus- |
| −0.02207 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.04317 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06222 | alt.negative.9:117568766, | | | | | | | | | | |
| 0.01489 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.0581 | alt.negative.9:117568766, | 3 | 0.31372549 | 0.52941176 | 0.49307692 | 0.5625 | 0.51428571 | 0.5625 | 0.34615385 | 0.72 | ileum.bma.model.clus- |
| 0.01387 | additive.9:117568766, | | | | | | | | | | ter.Entero_Clonocyte.RDS |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01249 | alt.negative.16:6147645, | | | | | | | | | | |
| −0.01279 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02864 | alt.negative.11:128443099, | | | | | | | | | | |
| −0.00625 | ref.negative.11:128443099, | | | | | | | | | | |
| 0 | ref.recessive.11:128443099, | | | | | | | | | | |
| 0 | haploinsuff11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.39215686 | 0.52941176 | 0.49846154 | 0.55 | 0.51612903 | 0.55 | 0.42307692 | 0.64 | ileum.bma.model.clus- |
| 0.04451 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.09495 | alt.negative.9:117568766, | | | | | | | | | | |
| 0.03015 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02588 | alt.negative.11:128443099, | | | | | | | | | | |
| −0.01411 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 3 | 0.33333333 | 0.50980392 | 0.43384615 | 0.52941176 | 0.5 | 0.52941176 | 0.34615385 | 0.68 | ileum.bma.model.clus- |
| −0.01563 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.06442 | alt.negative.9:117568766, | | | | | | | | | | |
| 0.02148 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02364 | alt.negative.16:6147645, | | | | | | | | | | |
| −0.01851 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 3 | 0.33333333 | 0.50980392 | 0.48846154 | 0.52941176 | 0.5 | 0.52941176 | 0.34615385 | 0.68 | ileum.bma.model.clus- |
| 0.04871 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06997 | ref.negative.16:11331509, | | | | | | | | | | |
| 0 | alt.recessive.16:11331509, | | | | | | | | | | |
| −0.0181 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0672 | alt.negative.9:117568766, | | | | | | | | | | |
| 0.02043 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.00531 | alt.recessive.21:45658474, | 3 | 0.33333333 | 0.50980392 | 0.52 | 0.52941176 | 0.5 | 0.52941176 | 0.34615385 | 0.68 | ileum.bma.model.clus- |
| −0.00088 | additive.21:45658474, | | | | | | | | | | ter.Entero_Clonocyte.RDS |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00384 | alt.recessive.5:11562221, | | | | | | | | | | |
| −0.00057 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 1e−04 | alt.negative.16:6147645 | | | | | | | | | | |
| −0.00504 | alt.recessive.21:45658474, | 3 | 0.33333333 | 0.50980392 | 0.54923077 | 0.52941176 | 0.5 | 0.52941176 | 0.34615385 | 0.68 | ileum.bma.model.clus- |
| −0.00093 | additive.21:45658474, | | | | | | | | | | ter.Entero_Clonocyte.RDS |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| 0.00105 | additive.9:117568766, | | | | | | | | | | |
| 0.00144 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| −0.00039 | alt.negative.16:6147645 | | | | | | | | | | |
| 0 | ref.negative.14:98734567, | 3 | 0.37254902 | 0.50980392 | 0.50538462 | 0.52631579 | 0.5 | 0.52631579 | 0.38461538 | 0.64 | ileum.bma.model.clus- |
| −0.01001 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05929 | ref.negative.16:11331509, | | | | | | | | | | |
| 0 | alt.recessive.16:11331509, | | | | | | | | | | |
| −0.01677 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0554 | alt.negative.9:117568766, | | | | | | | | | | |
| 0.01785 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.06413 | ref.negative.16:11331509, | 3 | 0.37254902 | 0.50980392 | 0.48461538 | 0.52631579 | 0.5 | 0.52631579 | 0.38461538 | 0.64 | ileum.bma.model.clus- |
| 0 | alt.recessive.16:11331509, | | | | | | | | | | ter.Entero_Clonocyte.RDS |
| −0.01723 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0808 | alt.negative.9:117568766, | | | | | | | | | | |
| 0.02554 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02458 | alt.negative.16:6147645, | | | | | | | | | | |
| −0.01674 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.00495 | alt.recessive.14:98734567, | 3 | 0.41176471 | 0.50980392 | 0.52769231 | 0.52380952 | 0.5 | 0.52380952 | 0.42307692 | 0.6 | ileum.bma.model.clus- |
| 0 | ref.negative.14:98734567, | | | | | | | | | | ter.Entero_Clonocyte.RDS |
| −0.005 | alt.recessive.21:45658474, | | | | | | | | | | |
| −0.00081 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| 0.00018 | altnegative.16:6147645 | | | | | | | | | | |
| −0.0031 | alt.recessive.5:11562221, | 3 | 0.41176471 | 0.50980392 | 0.50384615 | 0.52380952 | 0.5 | 0.52380952 | 0.42307692 | 0.6 | ileum.bma.model.clus- |
| −0.00054 | additive.5:11562221, | | | | | | | | | | ter.Entero_Clonocyte.RDS |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00048 | additive.9:117568766, | | | | | | | | | | |
| 0.00309 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| −0.00015 | alt.negative.16:6147645 | | | | | | | | | | |
| 0 | ref.negative.14:98734567, | 3 | 0.47058824 | 0.49019608 | 0.45 | 0.5 | 0.48148148 | 0.5 | 0.46153846 | 0.52 | ileum.bma.model.clus- |
| −0.02094 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.04328 | ref.recessive.21:45658474, | | | | | | | | | | |
| 0 | alt.negative.21:45658474, | | | | | | | | | | |
| −0.02443 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05928 | ref.negative.20:62305274, | | | | | | | | | | |
| 0 | alt.recessive.20:62305274, | | | | | | | | | | |
| 0.01623 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274 | | | | | | | | | | |
| 0 | ref.negative.14:98734567, | 3 | 0.35294118 | 0.49019608 | 0.47307692 | 0.5 | 0.48484848 | 0.5 | 0.34615385 | 0.64 | ileum.bma.model.clus- |
| −0.02123 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05268 | ref.recessive.21:45658474, | | | | | | | | | | |
| 0 | alt.negative.21:45658474, | | | | | | | | | | |
| −0.01449 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05436 | ref.negative.16:11331509, | | | | | | | | | | |
| 0 | alt.recessive.16:11331509, | | | | | | | | | | |
| −0.01805 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 3 | 0.31372549 | 0.49019608 | 0.47846154 | 0.5 | 0.48571429 | 0.5 | 0.30769231 | 0.68 | ileum.bma.model.clus- |
| −0.02073 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.04653 | ref.recessive.21:45658474, | | | | | | | | | | |
| 0 | alt.negative.21:45658474, | | | | | | | | | | |
| −0.01509 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.00743 | alt.negative.11:128443099, | | | | | | | | | | |
| −0.01684 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 3 | 0.50980392 | 0.49019608 | 0.50307692 | 0.5 | 0.48 | 0.5 | 0.5 | 0.48 | ileum.bma.model.clus- |
| −0.02673 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.07565 | alt.negative.9:117568766, | | | | | | | | | | |
| 0.02649 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02241 | alt.negative.11:128443099, | | | | | | | | | | |
| 0.00202 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.39215686 | 0.49019608 | 0.47076923 | 0.5 | 0.48387097 | 0.5 | 0.38461538 | 0.6 | ileum.bma.model.clus- |
| 0.04939 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05981 | ref.negative.16:11331509, | | | | | | | | | | |
| 0 | alt.recessive.16:11331509, | | | | | | | | | | |
| −0.01828 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01308 | alt.negative.11:128443099, | | | | | | | | | | |
| −0.01399 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.39215686 | 0.49019608 | 0.46 | 0.5 | 0.48387097 | 0.5 | 0.38461538 | 0.6 | ileum.bma.model.clus- |
| 0.05527 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.07884 | alt.negative.9:117568766, | | | | | | | | | | |
| 0.02106 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.03954 | alt.negative.16:6147645, | | | | | | | | | | |
| −0.01848 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 3 | 0.43137255 | 0.49019608 | 0.46 | 0.5 | 0.48275862 | 0.5 | 0.42307692 | 0.56 | ileum.bma.model.clus- |
| 0.03549 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.01921 | alt.negative.16:6147645, | | | | | | | | | | |
| −0.01098 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02091 | alt.negative.11:128443099, | | | | | | | | | | |
| 0.01151 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.06111 | ref.recessive.21:45658474, | 3 | 0.35294118 | 0.49019608 | 0.47923077 | 0.5 | 0.48484848 | 0.5 | 0.34615385 | 0.64 | ileum.bma.model.clus- |
| 0 | alt.negative.21:45658474, | | | | | | | | | | ter.Entero_Clonocyte.RDS |
| −0.00897 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05732 | ref.negative.16:11331509, | | | | | | | | | | |
| 0 | alt.recessive.16:11331509, | | | | | | | | | | |
| −0.01444 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.0567 | alt.negative.9:117568766, | | | | | | | | | | |
| 0.00751 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.06389 | ref.negative.16:11331509, | 3 | 0.31372549 | 0.49019608 | 0.51230769 | 0.5 | 0.48571429 | 0.5 | 0.30769231 | 0.68 | ileum.bma.model.clus- |
| 0 | alt.recessive.16:11331509, | | | | | | | | | | ter.Entero_Clonocyte.RDS |
| −0.03569 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.07005 | alt.negative.9:117568766, | | | | | | | | | | |
| 0.01756 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.02952 | alt.negative.11:128443099, | | | | | | | | | | |
| −0.00693 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00528 | alt.recessive.14:98734567, | 3 | 0.39215686 | 0.49019608 | 0.51615385 | 0.5 | 0.48387097 | 0.5 | 0.38461538 | 0.6 | ileum.bma.model.clus- |
| 0 | ref.negative.14:98734567, | | | | | | | | | | ter.Entero_Clonocyte.RDS |
| −0.00389 | alt.recessive.5:11562221, | | | | | | | | | | |
| −0.00021 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00064 | additive.9:117568766, | | | | | | | | | | |
| 0.00196 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766 | | | | | | | | | | |
| −0.00574 | alt.recessive.14:98734567, | 3 | 0.58823529 | 0.49019608 | 0.52 | 0.5 | 0.47619048 | 0.5 | 0.57692308 | 0.4 | ileum.bma.model.clus- |
| 0 | ref.negative.14:98734567, | | | | | | | | | | ter.Entero_Clonocyte.RDS |
| −0.00391 | alt.recessive.5:11562221, | | | | | | | | | | |
| −0.00027 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00054 | alt.negative.16:6147645 | | | | | | | | | | |
| −0.00524 | alt.recessive.14:98734567, | 3 | 0.43137255 | 0.49019608 | 0.52461538 | 0.5 | 0.48275862 | 0.5 | 0.42307692 | 0.56 | ileum.bma.model.clus- |
| 0 | ref.negative.14:98734567, | | | | | | | | | | ter.Entero_Clonocyte.RDS |
| 0.00079 | additive.9:117568766, | | | | | | | | | | |
| 0.00156 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766, | | | | | | | | | | |
| 8e−05 | alt.negative.16:6147645 | | | | | | | | | | |
| 0 | ref.negative.14:98734567, | 3 | 0.56862745 | 0.47058824 | 0.49538462 | 0.48275862 | 0.45454545 | 0.48275862 | 0.53846154 | 0.4 | ileum.bma.model.clus- |
| −0.0196 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03789 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.01791 | alt.negative.11:128443099, | | | | | | | | | | |
| 0.00063 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.45098039 | 0.47058824 | 0.47230769 | 0.47826087 | 0.46428571 | 0.47826087 | 0.42307692 | 0.52 | ileum.bma.model.clus- |
| 0.04221 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.04316 | ref.recessive.21:45658474, | | | | | | | | | | |
| 0 | alt.negative.21:45658474, | | | | | | | | | | |
| −0.01895 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06518 | ref.negative.16:11331509, | | | | | | | | | | |
| 0 | alt.recessive.16:11331509, | | | | | | | | | | |
| −0.02348 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0.04513 | ref.recessive.21:45658474, | 3 | 0.45098039 | 0.47058824 | 0.43307692 | 0.47826087 | 0.46428571 | 0.47826087 | 0.42307692 | 0.52 | ileum.bma.model.clus- |
| 0 | alt.negative.21:45658474, | | | | | | | | | | ter.Entero_Clonocyte.RDS |
| −0.01395 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05963 | ref.negative.20:62305274, | | | | | | | | | | |
| 0 | alt.recessive.20:62305274, | | | | | | | | | | |
| 0.02038 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06301 | ref.negative.16:11331509, | | | | | | | | | | |
| 0 | alt.recessive.16:11331509, | | | | | | | | | | |
| −0.01213 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 3 | 0.41176471 | 0.47058824 | 0.47384615 | 0.47619048 | 0.46666667 | 0.47619048 | 0.38461538 | 0.56 | ileum.bma.model.clus- |
| −0.01415 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03999 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06856 | ref.negative.16:11331509, | | | | | | | | | | |
| 0 | alt.recessive.16:11331509, | | | | | | | | | | |
| −0.01622 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.14:98734567, | 3 | 0.37254902 | 0.47058824 | 0.50153846 | 0.47368421 | 0.46875 | 0.47368421 | 0.34615385 | 0.6 | ileum.bma.model.clus- |
| −0.02014 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.0398 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.02691 | ref.recessive.21:45658474, | | | | | | | | | | |
| 0 | alt.negative.21:45658474, | | | | | | | | | | |
| −0.03186 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474 | | | | | | | | | | |
| 0 | ref.negative.5:11562221, | 3 | 0.37254902 | 0.47058824 | 0.43384615 | 0.47368421 | 0.46875 | 0.47368421 | 0.34615385 | 0.6 | ileum.bma.model.clus- |
| 0.04311 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06231 | ref.recessive.21:45658474, | | | | | | | | | | |
| 0 | alt.negative.21:45658474, | | | | | | | | | | |
| −0.00719 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.02732 | ref.negative.20:62305274, | | | | | | | | | | |
| 0 | alt.recessive.20:62305274, | | | | | | | | | | |
| 0.0194 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274 | | | | | | | | | | |
| 0 | ref.negative.5:11562221, | 3 | 0.37254902 | 0.47058824 | 0.48769231 | 0.47368421 | 0.46875 | 0.47368421 | 0.34615385 | 0.6 | ileum.bma.model.clus- |
| 0.04353 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.05944 | ref.recessive.21:45658474, | | | | | | | | | | |
| 0 | alt.negative.21:45658474, | | | | | | | | | | |
| −0.00884 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06574 | alt.negative.9:117568766, | | | | | | | | | | |
| 0.00716 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.5:11562221, | 3 | 0.37254902 | 0.47058824 | 0.47307692 | 0.47368421 | 0.46875 | 0.47368421 | 0.34615385 | 0.6 | ileum.bma.model.clus- |
| 0.04624 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06743 | ref.recessive.21:45658474, | | | | | | | | | | |
| 0 | alt.negative.21:45658474, | | | | | | | | | | |
| −6e−05 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.00335 | alt.negative.11:128443099, | | | | | | | | | | |
| −0.01938 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.07641 | ref.recessive.21:45658474, | 3 | 0.37254902 | 0.47058824 | 0.40461538 | 0.47368421 | 0.46875 | 0.47368421 | 0.34615385 | 0.6 | ileum.bma.model.clus- |
| 0 | alt.negative.21:45658474, | | | | | | | | | | ter.Entero_Clonocyte.RDS |
| 0.00736 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.04648 | ref.negative.20:62305274, | | | | | | | | | | |
| 0 | alt.recessive.20:62305274, | | | | | | | | | | |
| 0.01251 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07252 | alt.negative.9:117568766, | | | | | | | | | | |
| 0.012 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 3 | 0.33333333 | 0.47058824 | 0.40615385 | 0.47058824 | 0.47058824 | 0.47058824 | 0.30769231 | 0.64 | ileum.bma.model.clus- |
| −0.02611 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.06794 | ref.negative.20:62305274, | | | | | | | | | | |
| 0 | alt.recessive.20:62305274, | | | | | | | | | | |
| 0.01145 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.07137 | alt.negative.9:117568766, | | | | | | | | | | |
| 0.02064 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0.05952 | ref.recessive.21:45658474, | 3 | 0.33333333 | 0.47058824 | 0.43307692 | 0.47058824 | 0.47058824 | 0.47058824 | 0.30769231 | 0.64 | ileum.bma.model.clus- |
| 0 | alt.negative.21:45658474, | | | | | | | | | | ter.Entero_Clonocyte.RDS |
| −0.00532 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.01667 | ref.negative.20:62305274, | | | | | | | | | | |
| 0 | alt.recessive.20:62305274, | | | | | | | | | | |
| 0.01504 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00254 | alt.negative.11:128443099, | | | | | | | | | | |
| −0.0113 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.05221 | ref.negative.16:11331509, | 3 | 0.33333333 | 0.47058824 | 0.49846154 | 0.47058824 | 0.47058824 | 0.47058824 | 0.30769231 | 0.64 | ileum.bma.model.clus- |
| 0 | alt.recessive.16:11331509, | | | | | | | | | | ter.Entero_Clonocyte.RDS |
| −0.00576 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00888 | alt.negative.16:6147645, | | | | | | | | | | |
| −0.00832 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02277 | alt.negative.11:128443099, | | | | | | | | | | |
| 0.00997 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.00518 | alt.recessive.21:45658474, | 3 | 0.43137255 | 0.45098039 | 0.51769231 | 0.45454545 | 0.44827586 | 0.45454545 | 0.38461538 | 0.52 | ileum.bma.model.clus- |
| −0.00093 | additive.21:45658474, | | | | | | | | | | ter.Entero_Clonocyte.RDS |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00391 | alt.recessive.5:11562221, | | | | | | | | | | |
| −0.00049 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221, | | | | | | | | | | |
| 0.00087 | additive.9:117568766, | | | | | | | | | | |
| 0.00176 | alt.negative.9:117568766, | | | | | | | | | | |
| 0 | ref.negative.9:117568766, | | | | | | | | | | |
| 0 | alt.recessive.9:117568766, | | | | | | | | | | |
| 0 | haploinsuff.9:117568766, | | | | | | | | | | |
| 0 | ref.recessive.9:117568766 | | | | | | | | | | |
| 0 | ref.negative.14:98734567, | 3 | 0.35294118 | 0.45098039 | 0.39846154 | 0.44444444 | 0.45454545 | 0.44444444 | 0.30769231 | 0.6 | ileum.bma.model.clus- |
| −0.02025 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03043 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06396 | ref.negative.20:62305274, | | | | | | | | | | |
| 0 | alt.recessive.20:62305274, | | | | | | | | | | |
| 0.01272 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274 | | | | | | | | | | |
| −0.00501 | alt.recessive.14:98734567, | 3 | 0.35294118 | 0.45098039 | 0.53076923 | 0.44444444 | 0.45454545 | 0.44444444 | 0.30769231 | 0.6 | ileum.bma.model.clus- |
| 0 | ref.negative.14:98734567, | | | | | | | | | | ter.Entero_Clonocyte.RDS |
| −0.00513 | alt.recessive.21:45658474, | | | | | | | | | | |
| −0.00085 | additive.21:45658474, | | | | | | | | | | |
| 0 | haploinsuff.21:45658474, | | | | | | | | | | |
| 0 | alt.negative.21:45658474, | | | | | | | | | | |
| 0 | ref.recessive.21:45658474, | | | | | | | | | | |
| 0 | ref.negative.21:45658474, | | | | | | | | | | |
| −0.00461 | alt.recessive.5:11562221, | | | | | | | | | | |
| −0.00026 | additive.5:11562221, | | | | | | | | | | |
| 0 | haploinsuff.5:11562221, | | | | | | | | | | |
| 0 | alt.negative.5:11562221, | | | | | | | | | | |
| 0 | ref.recessive.5:11562221, | | | | | | | | | | |
| 0 | ref.negative.5:11562221 | | | | | | | | | | |
| 0 | ref.negative.14:98734567, | 3 | 0.31372549 | 0.45098039 | 0.39 | 0.4375 | 0.45714286 | 0.4375 | 0.26923077 | 0.64 | ileum.bma.model.clus- |
| −0.02466 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.07325 | ref.negative.20:62305274, | | | | | | | | | | |
| 0 | alt.recessive.20:62305274, | | | | | | | | | | |
| 0.01297 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01901 | alt.negative.11:128443099, | | | | | | | | | | |
| 0.00971 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.04638 | ref.recessive.21:45658474, | 3 | 0.31372549 | 0.45098039 | 0.47076923 | 0.4375 | 0.45714286 | 0.4375 | 0.26923077 | 0.64 | ileum.bma.model.clus- |
| 0 | alt.negative.21:45658474, | | | | | | | | | | ter.Entero_Clonocyte.RDS |
| −0.01501 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06056 | ref.negative.16:11331509, | | | | | | | | | | |
| 0 | alt.recessive.16:11331509, | | | | | | | | | | |
| −0.02495 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.0091 | alt.negative.11:128443099, | | | | | | | | | | |
| −0.01427 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.04618 | ref.recessive.21:45658474, | 3 | 0.41176471 | 0.43137255 | 0.46 | 0.42857143 | 0.43333333 | 0.42857143 | 0.34615385 | 0.52 | ileum.bma.model.clus- |
| 0 | alt.negative.21:45658474, | | | | | | | | | | ter.Entero_Clonocyte.RDS |
| −0.02611 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06065 | ref.negative.16:11331509, | | | | | | | | | | |
| 0 | alt.recessive.16:11331509, | | | | | | | | | | |
| −0.01741 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.00454 | alt.negative.16:6147645, | | | | | | | | | | |
| −0.01453 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.37254902 | 0.43137255 | 0.48769231 | 0.42105263 | 0.4375 | 0.42105263 | 0.30769231 | 0.56 | ileum.bma.model.clus- |
| −0.01708 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.06681 | ref.recessive.21:45658474, | | | | | | | | | | |
| 0 | alt.negative.21:45658474, | | | | | | | | | | |
| −0.00312 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.06038 | alt.negative.9:117568766, | | | | | | | | | | |
| 0.01265 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| 0 | ref.negative.14:98734567, | 3 | 0.37254902 | 0.43137255 | 0.41846154 | 0.42105263 | 0.4375 | 0.42105263 | 0.30769231 | 0.56 | ileum.bma.model.clus- |
| −0.0184 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05939 | ref.negative.16:11331509, | | | | | | | | | | |
| 0 | alt.recessive.16:11331509, | | | | | | | | | | |
| −0.00779 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01913 | alt.negative.16:6147645, | | | | | | | | | | |
| −0.01823 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.37254902 | 0.43137255 | 0.45461538 | 0.42105263 | 0.4375 | 0.42105263 | 0.30769231 | 0.56 | ileum.bma.model.clus- |
| −0.014 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.01474 | alt.negative.16:6147645, | | | | | | | | | | |
| −0.01502 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.02037 | alt.negative.11:128443099, | | | | | | | | | | |
| 0.00687 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.37254902 | 0.43137255 | 0.41923077 | 0.42105263 | 0.4375 | 0.42105263 | 0.30769231 | 0.56 | ileum.bma.model.clus- |
| 0.04179 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06601 | ref.negative.20:62305274, | | | | | | | | | | |
| 0 | alt.recessive.20:62305274, | | | | | | | | | | |
| 0.01638 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.09777 | alt.negative.9:117568766, | | | | | | | | | | |
| 0.03466 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.07537 | ref.negative.20:62305274, | 3 | 0.37254902 | 0.43137255 | 0.42923077 | 0.42105263 | 0.4375 | 0.42105263 | 0.30769231 | 0.56 | ileum.bma.model.clus- |
| 0 | alt.recessive.20:62305274, | | | | | | | | | | ter.Entero_Clonocyte.RDS |
| 0.01801 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06285 | ref.negative.16:11331509, | | | | | | | | | | |
| 0 | alt.recessive.16:11331509, | | | | | | | | | | |
| −0.00898 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01806 | alt.negative.11:128443099, | | | | | | | | | | |
| −0.01066 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.39215686 | 0.41176471 | 0.43230769 | 0.4 | 0.41935484 | 0.4 | 0.30769231 | 0.52 | ileum.bma.model.clus- |
| 0.03842 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.05247 | ref.negative.16:11331509, | | | | | | | | | | |
| 0 | alt.recessive.16:11331509, | | | | | | | | | | |
| −0.01988 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.03171 | alt.negative.16:6147645, | | | | | | | | | | |
| −0.02735 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.35294118 | 0.41176471 | 0.38846154 | 0.38888889 | 0.42424242 | 0.38888889 | 0.26923077 | 0.56 | ileum.bma.model.clus- |
| −0.02096 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.07652 | ref.negative.20:62305274, | | | | | | | | | | |
| 0 | alt.recessive.20:62305274, | | | | | | | | | | |
| 0.01587 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01371 | alt.negative.16:6147645, | | | | | | | | | | |
| −0.01864 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.35294118 | 0.41176471 | 0.46692308 | 0.38888889 | 0.42424242 | 0.38888889 | 0.26923077 | 0.56 | ileum.bma.model.clus- |
| −0.02004 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.05749 | ref.negative.16:11331509, | | | | | | | | | | |
| 0 | alt.recessive.16:11331509, | | | | | | | | | | |
| −0.00978 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.01364 | alt.negative.11:128443099, | | | | | | | | | | |
| −0.00177 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0.07273 | ref.recessive.21:45658474, | 3 | 0.35294118 | 0.41176471 | 0.44615385 | 0.38888889 | 0.42424242 | 0.38888889 | 0.26923077 | 0.56 | ileum.bma.model.clus- |
| 0 | alt.negative.21:45658474, | | | | | | | | | | ter.Entero_Clonocyte.RDS |
| 0.00189 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07234 | alt.negative.9:117568766, | | | | | | | | | | |
| 0.01374 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01281 | alt.negative.11:128443099, | | | | | | | | | | |
| −0.01639 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.05966 | ref.negative.20:62305274, | 3 | 0.35294118 | 0.41176471 | 0.35769231 | 0.38888889 | 0.42424242 | 0.38888889 | 0.26923077 | 0.56 | ileum.bma.model.clus- |
| 0 | alt.recessive.20:62305274, | | | | | | | | | | ter.Entero_Clonocyte.RDS |
| 0.02115 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.01668 | alt.negative.16:6147645, | | | | | | | | | | |
| −0.01778 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.01081 | alt.negative.11:128443099, | | | | | | | | | | |
| −0.01382 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.5:11562221, | 3 | 0.50980392 | 0.37254902 | 0.39461538 | 0.38461538 | 0.36 | 0.38461538 | 0.38461538 | 0.36 | ileum.bma.model.clus- |
| 0.03679 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06169 | ref.negative.20:62305274, | | | | | | | | | | |
| 0 | alt.recessive.20:62305274, | | | | | | | | | | |
| 0.00816 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.0258 | alt.negative.11:128443099, | | | | | | | | | | |
| −0.00256 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| −0.07479 | ref.negative.20:62305274, | 3 | 0.41176471 | 0.39215686 | 0.42 | 0.38095238 | 0.4 | 0.38095238 | 0.30769231 | 0.48 | ileum.bma.model.clus- |
| 0 | alt.recessive.20:62305274, | | | | | | | | | | ter.Entero_Clonocyte.RDS |
| 0.00594 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0978 | alt.negative.9:117568766, | | | | | | | | | | |
| 0.03507 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01832 | alt.negative.11:128443099, | | | | | | | | | | |
| −0.00477 | ref.negative.11:128443099, | | | | | | | | | | |
| 0 | ref.recessive.11:128443099, | | | | | | | | | | |
| 0 | haploinsuff.11:128443099, | | | | | | | | | | |
| 0 | additive.11:128443099, | | | | | | | | | | |
| 0 | alt.recessive.11:128443099 | | | | | | | | | | |
| 0 | ref.negative.14:98734567, | 3 | 0.31372549 | 0.41176471 | 0.43076923 | 0.375 | 0.42857143 | 0.375 | 0.23076923 | 0.6 | ileum.bma.model.clus- |
| −0.01366 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0 | ref.negative.5:11562221, | | | | | | | | | | |
| 0.03997 | ref.recessive.5:11562221, | | | | | | | | | | |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.03051 | alt.negative.16:6147645, | | | | | | | | | | |
| −0.01943 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 3 | 0.31372549 | 0.41176471 | 0.42076923 | 0.375 | 0.42857143 | 0.375 | 0.23076923 | 0.6 | ileum.bma.model.clus- |
| 0.03261 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.0579 | ref.negative.20:62305274, | | | | | | | | | | |
| 0 | alt.recessive.20:62305274, | | | | | | | | | | |
| 0.01396 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06801 | ref.negative.16:11331509, | | | | | | | | | | |
| 0 | alt.recessive.16:11331509, | | | | | | | | | | |
| −0.02229 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0.02177 | ref.recessive.21:45658474, | 3 | 0.37254902 | 0.39215686 | 0.43923077 | 0.36842105 | 0.40625 | 0.36842105 | 0.26923077 | 0.52 | ileum.bma.model.clus- |
| 0 | alt.negative.21:45658474, | | | | | | | | | | ter.Entero_Clonocyte.RDS |
| −0.03723 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.07344 | ref.negative.20:62305274, | | | | | | | | | | |
| 0 | alt.recessive.20:62305274, | | | | | | | | | | |
| 0.01596 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.00969 | alt.negative.16:6147645, | | | | | | | | | | |
| −0.02187 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.07407 | ref.negative.20:62305274, | 3 | 0.37254902 | 0.39215686 | 0.35076923 | 0.36842105 | 0.40625 | 0.36842105 | 0.26923077 | 0.52 | ileum.bma.model.clus- |
| 0 | alt.recessive.20:62305274, | | | | | | | | | | ter.Entero_Clonocyte.RDS |
| 0.01593 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.0812 | alt.negative.9:117568766, | | | | | | | | | | |
| 0.02088 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.0235 | alt.negative.16:6147645, | | | | | | | | | | |
| −0.01789 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.33333333 | 0.39215686 | 0.43769231 | 0.35294118 | 0.41176471 | 0.35294118 | 0.23076923 | 0.56 | ileum.bma.model.clus- |
| −0.01738 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| 0.05382 | ref.recessive.21:45658474, | | | | | | | | | | |
| 0 | alt.negative.21:45658474, | | | | | | | | | | |
| −0.01225 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.02017 | alt.negative.16:6147645, | | | | | | | | | | |
| −0.01929 | additive.166147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.5:11562221, | 3 | 0.33333333 | 0.39215686 | 0.42846154 | 0.35294118 | 0.41176471 | 0.35294118 | 0.23076923 | 0.56 | ileum.bma.model.clus- |
| 0.0257 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| 0.06349 | ref.recessive.21:45658474, | | | | | | | | | | |
| 0 | alt.negative.21:45658474, | | | | | | | | | | |
| −0.00307 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.02186 | alt.negative.16:6147645, | | | | | | | | | | |
| −0.02625 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.06744 | ref.recessive.21:45658474, | 3 | 0.33333333 | 0.39215686 | 0.42692308 | 0.35294118 | 0.41176471 | 0.35294118 | 0.23076923 | 0.56 | ileum.bma.model.clus- |
| 0 | alt.negative.21:45658474, | | | | | | | | | | ter.Entero_Clonocyte.RDS |
| −0.00413 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| −0.05702 | alt.negative.9:117568766, | | | | | | | | | | |
| 0.00886 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766, | | | | | | | | | | |
| 0.01275 | alt.negative.16:6147645, | | | | | | | | | | |
| −0.02139 | additive.16:6147645, | | | | | | | | | | |
| 0 | ref.negative.l6:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| −0.08467 | ref.negative.20:62305274, | 3 | 0.33333333 | 0.39215686 | 0.40076923 | 0.35294118 | 0.41176471 | 0.35294118 | 0.23076923 | 0.56 | ileum.bma.model.clus- |
| 0 | alt.recessive.20:62305274, | | | | | | | | | | ter.Entero_Clonocyte.RDS |
| 0.02625 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06504 | ref.negative.16:11331509, | | | | | | | | | | |
| 0 | alt.recessive.16:11331509, | | | | | | | | | | |
| −0.00625 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| −0.08499 | alt.negative.9:117568766, | | | | | | | | | | |
| 0.02661 | additive.9:117568766, | | | | | | | | | | |
| 0 | ref.negative.9:117568766, | | | | | | | | | | |
| 0 | haploinsuff.9:117568766, | | | | | | | | | | |
| 0 | alt.recessive.9:117568766, | | | | | | | | | | |
| 0 | ref.recessive.9:117568766 | | | | | | | | | | |
| −0.08096 | ref.negative.20:62305274, | 3 | 0.33333333 | 0.39215686 | 0.36538462 | 0.35294118 | 0.41176471 | 0.35294118 | 0.23076923 | 0.56 | ileum.bma.model.clus- |
| 0 | alt.recessive.20:62305274, | | | | | | | | | | ter.Entero_Clonocyte.RDS |
| 0.02412 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06496 | ref.negative.16:11331509, | | | | | | | | | | |
| 0 | alt.recessive.16:11331509, | | | | | | | | | | |
| −0.00655 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509, | | | | | | | | | | |
| 0.02183 | alt.negative.16:6147645, | | | | | | | | | | |
| −0.02259 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0 | ref.negative.14:98734567, | 3 | 0.39215686 | 0.37254902 | 0.38 | 0.35 | 0.38709677 | 0.35 | 0.26923077 | 0.48 | ileum.bma.model.clus- |
| −0.01696 | ref.recessive.14:98734567, | | | | | | | | | | ter.Entero_Clonocyte.RDS |
| 0 | alt.negative.14:98734567, | | | | | | | | | | |
| 0 | haploinsuff.14:98734567, | | | | | | | | | | |
| 0 | additive.14:98734567, | | | | | | | | | | |
| −0.06552 | ref.negative.20:62305274, | | | | | | | | | | |
| 0 | alt.recessive.20:62305274, | | | | | | | | | | |
| 0.01 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| −0.06675 | ref.negative.16:11331509, | | | | | | | | | | |
| 0 | alt.recessive.16:11331509, | | | | | | | | | | |
| −0.00451 | alt.negative.16:11331509, | | | | | | | | | | |
| 0 | ref.recessive.16:11331509, | | | | | | | | | | |
| 0 | haploinsuff.16:11331509, | | | | | | | | | | |
| 0 | additive.16:11331509 | | | | | | | | | | |
| 0 | ref.negative.5:11562221, | 3 | 0.37254902 | 0.35294118 | 0.33846154 | 0.31578947 | 0.375 | 0.31578947 | 0.23076923 | 0.48 | ileum.bma.model.clus- |
| 0.0394 | ref.recessive.5:11562221, | | | | | | | | | | ter.Entero_Clonocyte.RDS |
| 0 | haploinsuff.5:11562221, | | | | | | | | | | |
| 0 | additive.5:11562221, | | | | | | | | | | |
| 0 | alt.negative.5:11562221, | | | | | | | | | | |
| −0.06701 | ref.negative.20:62305274, | | | | | | | | | | |
| 0 | alt.recessive.20:62305274, | | | | | | | | | | |
| 0.01984 | ref.recessive.20:62305274, | | | | | | | | | | |
| 0 | alt.negative.20:62305274, | | | | | | | | | | |
| 0 | haploinsuff.20:62305274, | | | | | | | | | | |
| 0 | additive.20:62305274, | | | | | | | | | | |
| 0.03553 | alt.negative.16:6147645, | | | | | | | | | | |
| −0.02756 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645 | | | | | | | | | | |
| 0.04604 | ref.recessive.21:45658474, | 3 | 0.31372549 | 0.37254902 | 0.46 | 0.3125 | 0.4 | 0.3125 | 0.19230769 | 0.56 | ileum.bma.model.clus- |
| 0 | alt.negative.21:45658474, | | | | | | | | | | ter.Entero_Clonocyte.RDS |
| −0.01737 | additive.21:45658474, | | | | | | | | | | |
| 0 | ref.negative.21:45658474, | | | | | | | | | | |
| 0 | haploinsuff.21:45658474, | | | | | | | | | | |
| 0 | alt.recessive.21:45658474, | | | | | | | | | | |
| 0.01234 | alt.negative.16:6147645, | | | | | | | | | | |
| −0.01828 | additive.16:6147645, | | | | | | | | | | |
| 0 | ref.negative.16:6147645, | | | | | | | | | | |
| 0 | alt.recessive.16:6147645, | | | | | | | | | | |
| 0 | ref.recessive.16:6147645, | | | | | | | | | | |
| 0 | haploinsuff.16:6147645, | | | | | | | | | | |
| 0.00513 | alt.negative.11:128443099, | | | | | | | | | | |
| −0.01386 | ref.negative.11:128443099, | | | | | | | | | | |
| 0 | ref.recessive.11:128443099, | | | | | | | | | | |
| 0 | haploinsuff11:128443099, | | | | | | | | | | |
| 0 | additive.11:128443099, | | | | | | | | | | |
| 0 | alt.recessive.11:128443099 | | | | | | | | | | |
| −0.00413167705741463 | alt.recessive.14:98734567, | 5 | 0.33333333 | 0.50980392 | 0.56923077 | 0.52941176 | 0.5 | 0.52941176 | 0.34615385 | 0.68 | ileum.bma.model.clus- |
| −0.0044359554292331 | alt.recessive.21:45658474, | | | | | | | | | | ter.Enterocyte.RDS |
| −0.00250017985876822 | alt.recessive.5:11562221, | | | | | | | | | | |
| 0.000650367063738027 | additive.9:117568766, | | | | | | | | | | |
| −0.000997363768312975 | additive.21:45658474, | | | | | | | | | | |
| 0.000657529290080377 | alt.negative.9:117568766, | | | | | | | | | | |
| −0.0011883988810037 | additive.5:11562221, | | | | | | | | | | |
| 0.000767215741330144 | haploinsuff.5:11562221, | | | | | | | | | | |
| −3.83886285000346e−05 | alt.negative.5:11562221, | | | | | | | | | | |
| 0.000308632211177067 | haploinsuff.21:45658474, | | | | | | | | | | |
| −0.000306844405921009 | ref.negative.9:117568766, | | | | | | | | | | |
| 0.000212437039615959 | alt.recessive.9:117568766, | | | | | | | | | | |
| 0.0001621570612628 | haploinsuff.9:117568766, | | | | | | | | | | |
| −0.000285045293093247 | ref.recessive.5:11562221, | | | | | | | | | | |
| −0.000243858655992485 | alt.negative.21:45658474, | | | | | | | | | | |
| −3.17901237909852e−06 | ref.recessive.21:45658474, | | | | | | | | | | |
| −0.000332281196715872 | ref.recessive.9:117568766, | | | | | | | | | | |
| 0.000647604076515593 | ref.negative.14:98734567, | | | | | | | | | | |
| 2.56340836167717e−05 | alt.negative.16:6147645, | | | | | | | | | | |
| 0.0003441190525664 | ref.negative.5:11562221, | | | | | | | | | | |
| 0.000194490403126353 | ref.negative.21:45658474 | | | | | | | | | | |
| 0.00126367892280255 | ref.negative.5:11562221, | 4 | 0.70588235 | 0.64705882 | 0.65 | 0.61111111 | 0.73333333 | 0.61111111 | 0.84615385 | 0.44 | ileum.bma.model.cluster.moDC.RDS |
| 0.000385412296568075 | ref.negative.21:45658474, | | | | | | | | | | |
| 0.00107853624983427 | ref.negative.14:98734567, | | | | | | | | | | |
| 0.000864588647734689 | ref.negative.20:62305274, | | | | | | | | | | |
| −0.000778141022430513 | alt.recessive.20:62305274, | | | | | | | | | | |
| −0.000751986747075365 | alt.recessive.14:98734567, | | | | | | | | | | |
| −0.00033387140950681 | alt.recessive.21:45658474, | | | | | | | | | | |
| −0.000707982569494113 | alt.recessive.5:11562221 | | | | | | | | | | |
| 0.00198 | ref.negative.5:11562221, | 4 | 0.70588235 | 0.64705882 | 0.65 | 0.61111111 | 0.73333333 | 0.61111111 | 0.84615385 | 0.44 | ileum.bma.model.cluster.moDC.RDS |
| 0 | alt.recessive.5:11562221, | | | | | | | | | | |
| 0.00072 | ref.negative.21:45658474, | | | | | | | | | | |
| 0 | alt.recessive.21:45658474, | | | | | | | | | | |
| 0.00183 | ref.negative.14:98734567, | | | | | | | | | | |
| 0 | alt.recessive.14:98734567, | | | | | | | | | | |
| 0.00165 | refnegative.20:62305274, | | | | | | | | | | |
| 0 | alt.recessive.20:62305274 | | | | | | | | | | |
| 0.00065 | ref.negative.21:45658474, | 3 | 0.7254902 | 0.62745098 | 0.62692308 | 0.59459459 | 0.71428571 | 0.59459459 | 0.84615385 | 0.4 | ileum.bma.model.cluster.moDC.RDS |
| 0 | alt.recessive.21:45658474, | | | | | | | | | | |
| 0.00177 | ref.negative.14:98734567, | | | | | | | | | | |
| 0 | alt.recessive.14:98734567, | | | | | | | | | | |
| 0.00157 | ref.negative.20:62305274, | | | | | | | | | | |
| 0 | alt.recessive.20:62305274 | | | | | | | | | | |
| 0.00188 | ref.negative.5:11562221, | 3 | 0.76470588 | 0.62745098 | 0.62538462 | 0.58974359 | 0.75 | 0.58974359 | 0.88461538 | 0.36 | ileum.bma.model.cluster.moDC.RDS |
| 0 | alt.recessive.5:11562221, | | | | | | | | | | |
| 0.00079 | ref.negative.21:45658474, | | | | | | | | | | |
| 0 | alt.recessive.21:45658474, | | | | | | | | | | |
| 0.00155 | ref.negative.20:62305274, | | | | | | | | | | |
| 0 | alt.recessive.20:62305274 | | | | | | | | | | |
| 0.0018 | ref.negative.5:11562221, | 3 | 0.78431373 | 0.60784314 | 0.60692308 | 0.575 | 0.72727273 | 0.575 | 0.88461538 | 0.32 | ileum.bma.model.cluster.moDC.RDS |
| 0 | alt.recessive.5:11562221, | | | | | | | | | | |
| 0.00056 | refnegative.21:45658474, | | | | | | | | | | |
| 0 | alt.recessive.21:45658474, | | | | | | | | | | |
| 0.00169 | ref.negative.14:98734567, | | | | | | | | | | |
| 0 | alt.recessive.14:98734567 | | | | | | | | | | |
| 0.00189 | ref.negative.5:11562221, | 3 | 0.82352941 | 0.60784314 | 0.60230769 | 0.57142857 | 0.77777778 | 0.57142857 | 0.92307692 | 0.28 | ileum.bma.model.cluster.moDC.RDS |
| 0 | alt.recessive.5:11562221, | | | | | | | | | | |
| 0.00189 | refnegative.14:98734567, | | | | | | | | | | |
| 0 | alt.recessive.14:98734567, | | | | | | | | | | |
| 0.00156 | refnegative.20:62305274, | | | | | | | | | | |
| 0 | alt.recessive.20:62305274 | | | | | | | | | | |

Lengthy table referenced here
US20260066079A1-20260305-T00002
Please refer to the end of the specification for access instructions.

As is clear from the above description, β₀ is the intercept used in MRS calculation and can move the resulting MRS up or down. In some embodiments of the various methods provided herein, the β₀ used for the calculation of MRS, RPS, and/or PRI is about 0.0077127943934849. In some embodiments, the β₀ used for the calculation of MRS, RPS, and/or PRI is 0.0077127943934849. In some embodiments, the β₀ used for the calculation of MRS, RPS, and/or PRI is about 0.008. In some embodiments, the cutoff for MRS is about 0.0322446725024791 and β₀ is about 0.0077127943934849. In some embodiments, the cutoff for MRS is 0.0322446725024791 and β₀ is about 0.0077127943934849. In some embodiments, the cutoff for MRS is about 0.03 and β₀ is about 0.0077127943934849. In some embodiments, the cutoff for MRS is about 0.0322446725024791 and β₀ is about 0.008. In some embodiments, the cutoff for MRS is 0.0322446725024791 and β₀ is about 0.008. In some embodiments, the cutoff for MRS is about 0.03 and β₀ is about 0.008. In some embodiments, the β₀ and the MRS cutoff described in this paragraph is provided for each row of Table 31.

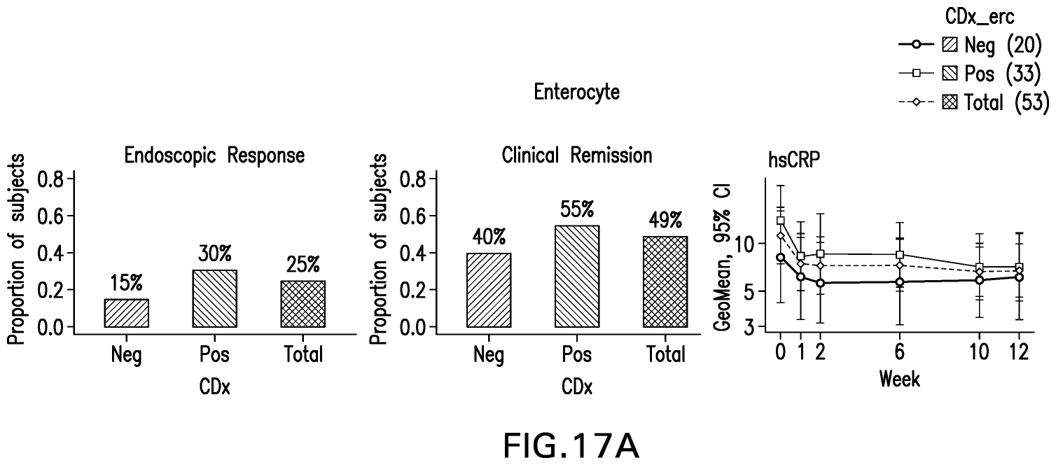

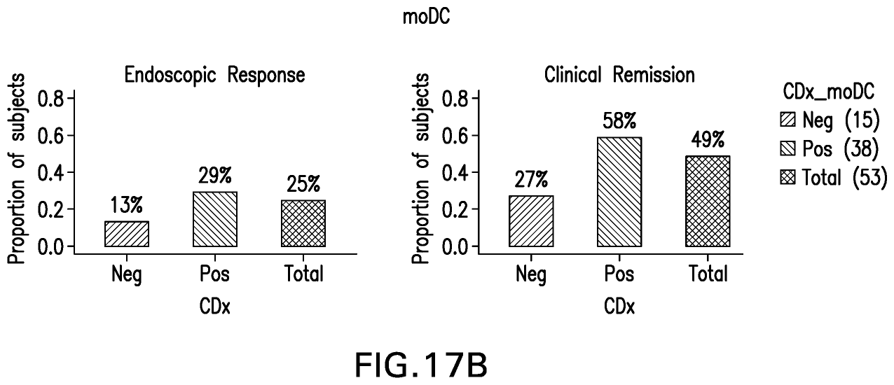

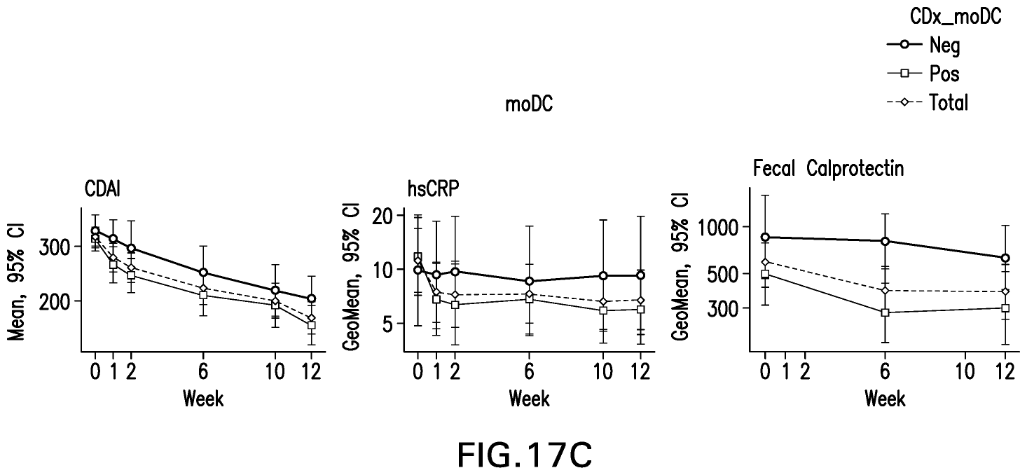

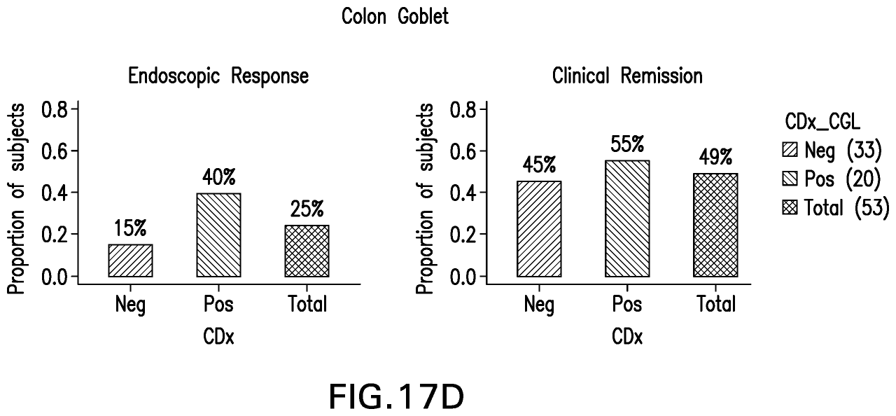

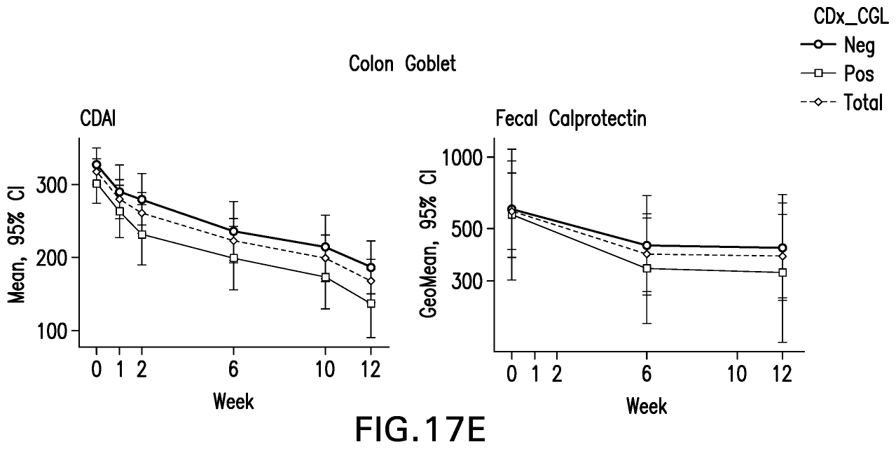

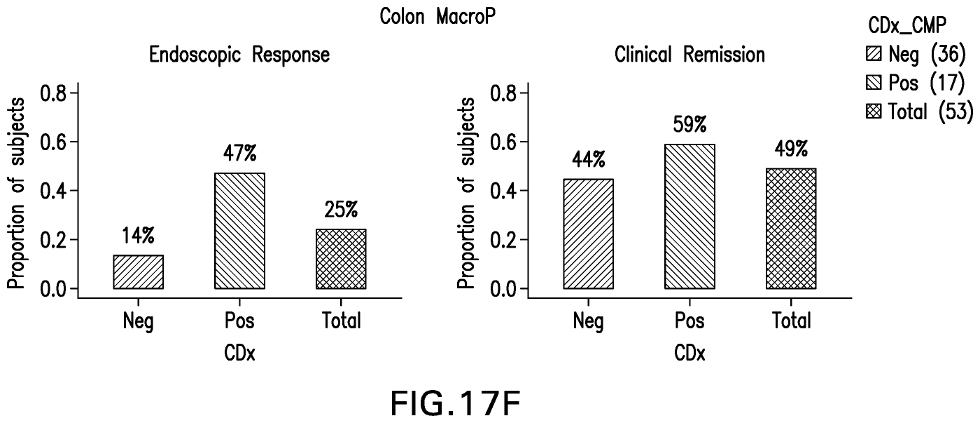

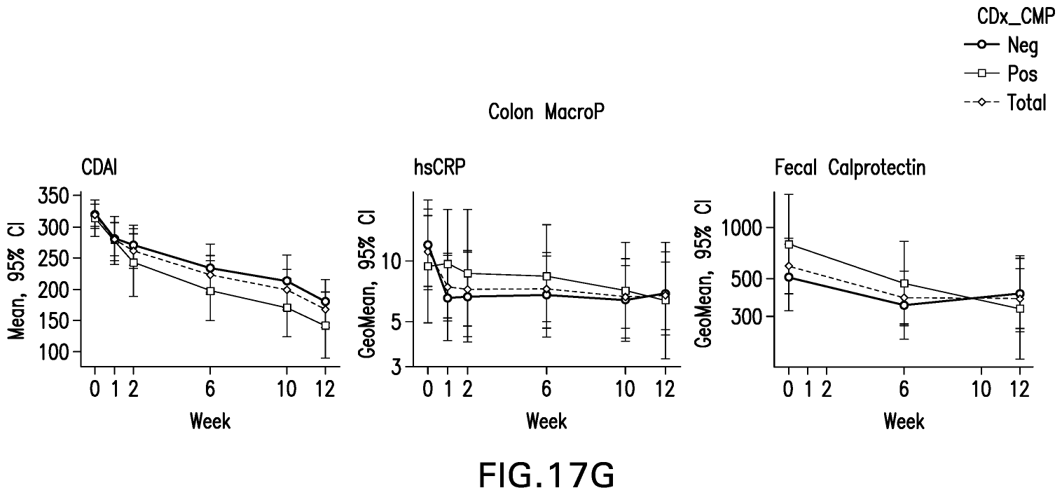

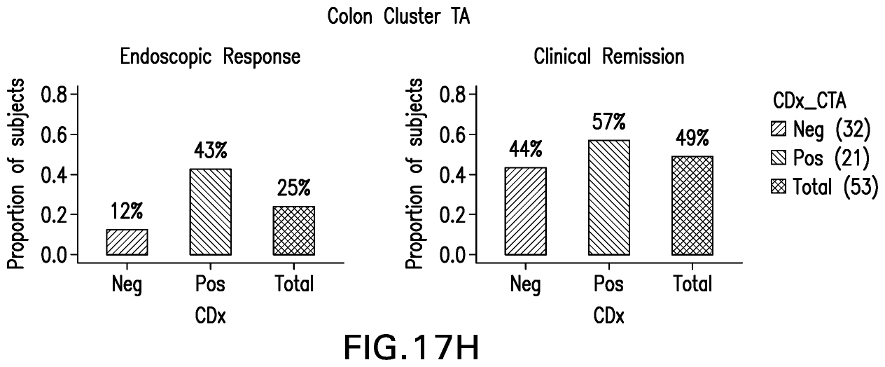

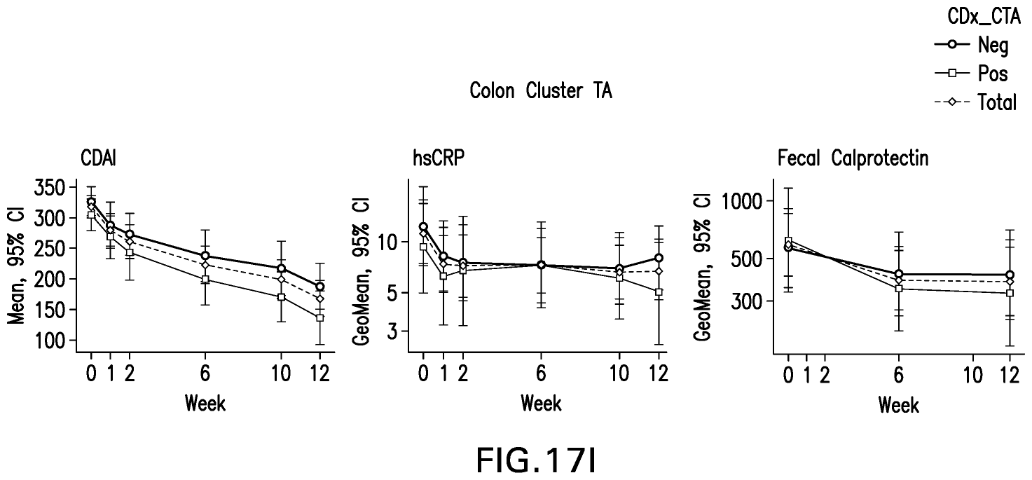

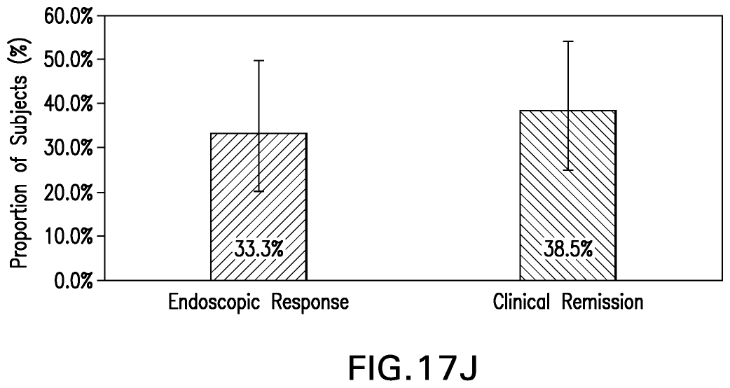

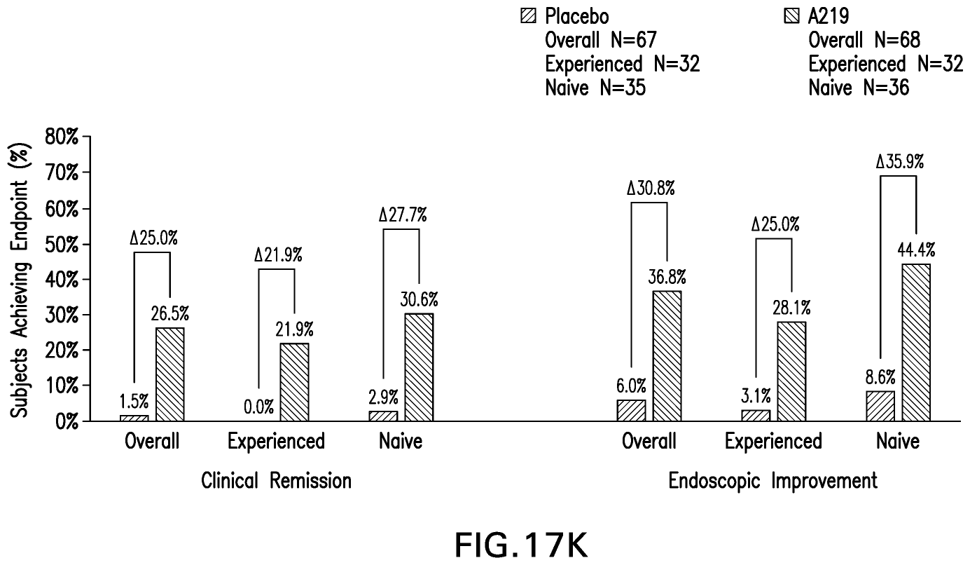

Certain exemplary comparisons of clinical remission and endoscopic improvement between CDx classified patients and all patients for the combined average CDx models are shown in FIG. 17A-17I. Comparisons with certain combined average CDx models (last 19 rows of Table 31) were shown to provide an overall picture of the models based on certain predictive cell types and to evaluate these combined average models. As shown in FIG. 17A, the combined average CDx model for predicting altered regulation of ileum entero_clonocytes enriched patients having endoscopic response and clinical remission among the CDx positive subpopulation, increasing endoscopic response from 25% in all patients to 30% in CDx positive patients and increasing clinical remission from 49% in all patients to 55% in CDx positive patients. Furthermore, the combined average CDx model for predicting altered regulation of entero_clonocytes also distinguished the secondary biomarkers for clinical improvement, decreasing the mean hsCRP level (as defined in secondary objective in Table 32) in CDx positive patients while increasing the mean hsCRP in CDx negative patients (FIG. 17A).

As shown in FIG. 17B, the combined average CDx model for predicting altered regulation of colon moDC enriched patients having endoscopic response and clinical remission among the CDx positive subpopulation, increasing endoscopic response from 25% in all patients to 29% in CDx positive patients and increasing clinical remission from 49% in all patients to 58% in CDx positive patients. Furthermore, as shown in FIG. 17C, the combined average CDx model for predicting altered regulation of colon moDC also distinguished the secondary biomarkers for clinical improvement, decreasing the mean CDAI, mean fecal calprotectin, and mean hsCRP level (each as defined in secondary objective in Table 32) in CDx positive patients while increasing the same parameters in CDx negative patients.

As shown in FIG. 17D, the combined average CDx model for predicting altered regulation of colon goblet cells enriched patients having endoscopic response and clinical remission among the CDx positive subpopulation, increasing endoscopic response from 25% in all patients to 40% in CDx positive patients and increasing clinical remission from 49% in all patients to 55% in CDx positive patients. Furthermore, as shown in FIG. 17E, the combined average CDx model for predicting altered regulation of colon goblet cells also distinguished the secondary biomarkers for clinical improvement, decreasing mean fecal calprotectin and mean hsCRP level (each as defined in secondary objective in Table 32) in CDx positive patients while increasing the same parameters in CDx negative patients.

As shown in FIG. 17F, the combined average CDx model for predicting altered regulation of colon resident macrophage enriched patients having endoscopic response and clinical remission among the CDx positive subpopulation, increasing endoscopic response from 25% in all patients to 47% in CDx positive patients and increasing clinical remission from 49% in all patients to 59% in CDx positive patients. Furthermore, as shown in FIG. 17G, the combined average CDx model for predicting altered regulation of colon resident macrophage also distinguished the secondary biomarkers for clinical improvement, including mean CDAI, mean fecal calprotectin, and mean hsCRP level (each as defined in secondary objective in Table 32) in CDx positive patients when comparing against the same parameters in CDx negative patients.

As shown in FIG. 17H, the combined average CDx model for predicting altered regulation of colon transit-amplifying (TA) progenitor cells enriched patients having endoscopic response and clinical remission among the CDx positive subpopulation, increasing endoscopic response from 25% in all patients to 43% in CDx positive patients and increasing clinical remission from 49% in all patients to 57% in CDx positive patients. Furthermore, as shown in FIG. 17I, the combined average CDx model for predicting altered regulation of colon TA cells also distinguished the secondary biomarkers for clinical improvement, decreasing the mean CDAI, mean fecal calprotectin, and mean hsCRP level (each as defined in secondary objective in Table 32) in CDx positive patients while increasing the same parameters in CDx negative patients.

Additional criteria for selecting patients for improving the patient outcomes of anti-TL1A treatment in CD patients were also analyzed. Patients that have been treated with an advanced IBD therapy have a high robust rate, as measured by both clinical remission and endoscopic improvement (FIG. 17J). In FIG. 17J, the patients that have already been treated with an advanced IBD therapy were determined based on treatment with approved biologics (anti-TNFα, anti-IL23, or anti-integrin), S1P1 modulators, and/or JAK inhibitors prior to the treatment with A219.

Accordingly, a number of pre-specified CDx models (e.g. predictive of ileum entero-clonocytes, colon moDC, colon resident macrophages, colon goblet cells, and colon TA cells) from Table 31 were validated in clinical trials to identified a CDx positive subpopulation that have an increased proportion treated patients with endoscopic response and clinical remission, thereby confirming the CDx models provided herein with clinical data. Such enrichment of endoscopic response and clinical remission in CDx positive subpopulation was further validated by the distinctive secondary biomarkers between the CDx positive and CDx negative subpopulations. Therefore, the CDx models described herein provide both the rationale and mechanisms, i.e. the association between a particular cell types with IBD patients' response to anti-TL1A treatment, and provide the validated clinical effectiveness in enriching clinical remission and endoscopic improvement in CDx positive IBD patient subpopulation.

### 7.24 Example 24: Additional Selection Criteria for Treating UC Patients with Anti-TL1A

Having conducted the clinical trial described in Section 7.22 (Example 22), various criteria for selecting patients for improving the patient outcomes of anti-TL1A treatment in UC patients were analyzed. Patients that have not been treated with an advanced IBD therapy (“Naïve” in FIG. 17K) have a higher response rate than both the patients that have already been treated with an advanced IBD therapy (“Experienced” in FIG. 17K) and the overall patient population (both naïve and experienced, shown as “Overall” in FIG. 17K), as measured by both clinical remission and endoscopic improvement (FIG. 17K). In FIG. 17K, the patients that have already been treated with an advanced IBD therapy (“Experienced” in FIG. 17K) were determined based on treatment with approved biologics (anti-TNFα, anti-IL23, or anti-integrin), S1P1 modulators, and/or JAK inhibitors prior to the treatment with A219. The results in FIG. 17K demonstrate that no prior exposure to advanced IBD therapies can be a validated selection criterium to improve patients' response to anti-TL1A treatment in UC patients.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

LENGTHY TABLES
The patent application contains a lengthy table section. A copy of the table is available in electronic form from the USPTO web site (https://seqdata.uspto.gov/docdetail?docId=US20260066079A1). An electronic copy of the table will also be available from the USPTO upon request and payment of the fee set forth in 37 CFR 1.19(b)(3).

What is claimed:

1. A method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a Predictive Response Index (PRI) above a cutoff, wherein the PRI is calculated from a combination of polymorphisms determined from a sample from the subject and the PRI above the cutoff predicts a positive therapeutic response in the subject to a treatment with the inhibitor of TL1A activity or expression with a positive predictive value of at least about 29%.

2. A method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
(i) obtaining or having obtained a sample from the subject;
(ii) subjecting the sample to an assay adapted to detect a combination of polymorphisms;
(iii) calculating a Predictive Response Index (PRI) from the combination of polymorphisms, wherein the subject is determined to be suitable for treatment with an inhibitor of TL1A activity or expression if the PRI is above a cutoff; and
(b) treating the subject by administering a therapeutically effective amount of the inhibitor of TL1A activity or expression to the subject.

3. A method of determining a Predictive Response Index (PRI) for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the PRI above a cutoff indicates the subject is suitable for treatment with an inhibitor of TL1A activity or expression.

4. A method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression if the PRI is above a cutoff.

5. The method of any one of claims 2 to 4, wherein the method further comprises preparing DNA from the sample.

6. The method of any one of claims 1 to 5, wherein the PRI is a Response Probability Score (RPS).

7. The method of any one of claims 1 to 6, wherein the PRI has a positive correlation coefficient with RPS.

8. The method of claim 7, wherein the correlation coefficient is Pearson correlation coefficient or Spearman correlation coefficient.

9. The method of claim 7 or 8, wherein the positive correlation coefficient is at least about 0.6, at least about 0.65, at least about 0.7, at least about 0.75, at least about 0.8, at least about 0.85, at least about 0.95, at least about 0.99, or 1.

10. A computer-implemented method of determining a Response Probability Score (RPS) for a subject, the method comprising:
(a) receiving genotype data obtained from a sample from the subject with an inflammatory, a fibrotic, or a fibrostenotic disease or condition, wherein the genotype data comprises a combination of polymorphisms;
(b) analyzing the genotype data with a first statistical algorithm configured to produce a Model Risk Score (MRS) for the subject by performing operations comprising:
(i) assigning a weighted numerical value to each polymorphism in the combination of polymorphisms to produce a plurality of weighted values; and
(ii) summing the plurality of weighted values;
(c) providing the MRS to a second statistical algorithm configured to perform a logarithmic function on the MRS to produce a Response Probability Score (RPS); and
(d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition.

11. A computer-implemented method of determining a Response Probability Score (RPS) for a subject, the method comprising:
(a) obtaining a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
(b) receiving genotype data for a plurality of polymorphisms obtained from a sample from the subject;
(c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
(d) applying a logarithmic scale and a cutoff to the MRS to produce a Response Probability Score (RPS).

12. The method of any one of claims 6 to 11, wherein the RPS ranges from 0 to 1.

13. The method of any one of claims 6 to 12, wherein the cutoff is 0.5.

14. The method of any one of claims 6 to 13, wherein the RPS is calculated as 1/(1+e^{(-MRS)}), wherein the MRS is calculated as
$${β}_{0} + {\sum }_{i = 1}^{n} ( {β}_{i} ) \times ( {χ}_{i} ) ,$$
and wherein χᵢ is the mathematical representation of the ith single nucleotide polymorphisms (SNP) in the model and βᵢ is the weight for the ith SNP in the model.

15. The method of any one of claims 1 to 5, wherein PRI is a Model Risk Score (MRS).

16. The method of any one of claims 1 to 5 and 15, wherein the PRI has a positive correlation coefficient with MRS.

17. The method of claim 15 and 16, wherein the correlation coefficient is Pearson correlation coefficient or Spearman correlation coefficient.

18. The method of claim 16 and 17, wherein the positive correlation coefficient is at least about 0.6, at least about 0.65, at least about 0.7, at least about 0.75, at least about 0.8, at least about 0.85, at least about 0.95, at least about 0.99, or 1.

19. The method of any one of claims 10 to 16, wherein the MRS is calculated as
$${β}_{0} + {\sum }_{i = 1}^{n} ( {β}_{i} ) \times ( {χ}_{i} ) ,$$
and wherein χᵢ is the mathematical representation of the ith SNP in the model.

20. The method of claim 10 or 19, wherein the SNP in the model is mathematically represented by χᵢ as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and I for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

21. The method of any one of claims 1 to 20, wherein the combination of polymorphisms comprises one or more polymorphisms selected from Table 27, or a proxy polymorphism in linkage disequilibrium therewith as determined with an R² of at least 0.85, or a combination thereof.

22. The method of any one of claims 1 to 21, wherein the cutoff is such that the PRI above the cutoff is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%.

23. The method of any one of claims 1 to 22, wherein the cutoff is such that the PRI above the cutoff is predictive of a positive therapeutic response in the subject to a treatment with the inhibitor of TL1A activity or expression with a specificity of at least about 50%, 55%, 60%, 65%, 70%, 75%, 80%, 85%, 90%, or 95%.

24. The method of any one of claims 1 to 23, wherein the cutoff is such that the PRI above the cutoff is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%.

25. The method of any one of claims 1 to 24, wherein the cutoff is such that the PRI above the cutoff is predictive of a positive therapeutic response in the subject to a treatment with the inhibitor of TL1A activity or expression with a sensitivity of at least about 20%, 25%, 30%, 35%, 40%, 45%, 55%, 60%, 65%, 70%, 75%, 80%, 85%, 90%, or 95%.

26. The method of any one of claims 1 to 25, wherein the cutoff is such that the PRI above the cutoff is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%.

27. The method of any one of claims 1 to 26, wherein the cutoff is such that the PRI above the cutoff is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%.

28. The method of any one of claims 1 to 27, wherein the cutoff is such that the PRI above the cutoff is predictive of an increase of one or more IBD enriched cell types with a positive predictive value of at least about 29%, 30%, 35%, 40%, 45%, 50%, 51%, 55%, 60%, 65%, 70%, 75%, 80%, 85%, 90%, or 95%.

29. The method of any one of claims 1 to 28, wherein the cutoff is such that the PRI above the cutoff is predictive of a decrease of one or more IBD depleted cell types with a positive predictive value of at least about 29%, 30%, 35%, 40%, 45%, 50%, 51%, 55%, 60%, 65%, 70%, 75%, 80%, 85%, 90%, or 95%.

30. The method of any one of claims 1 to 29, wherein the cutoff is such that the PRI above the cutoff is predictive of an increase of one or more IBD enriched cell types with a specificity of at least about 50%, 55%, 60%, 65%, 70%, 75%, 80%, 85%, 90%, or 95%.

31. The method of any one of claims 1 to 30, wherein the cutoff is such that the PRI above the cutoff is predictive of a decrease of one or more IBD depleted cell types with a specificity of at least about 50%, 55%, 60%, 65%, 70%, 75%, 80%, 85%, 90%, or 95%.

32. The method of any one of claims 1 to 31, wherein the cutoff is such that the PRI above the cutoff is predictive of an increase of one or more IBD enriched cell types with a negative predictive value of at least about 50%, 55%, 60%, 65%, 70%, 75%, 80%, 85%, 90%, or 95%.

33. The method of any one of claims 1 to 31, wherein the cutoff is such that the PRI above the cutoff is predictive of a decrease of one or more IBD depleted cell types with a negative predictive value of at least about 50%, 55%, 60%, 65%, 70%, 75%, 80%, 85%, 90%, or 95%.

34. The method of any one of claims 1 to 33, wherein the cutoff is such that the PRI above the cutoff is predictive of an increase of one or more IBD enriched cell types with a sensitivity of at least about 20%, 25%, 30%, 35%, 40%, 45%, 50%, 55%, 60%, 65%, 70%, 75%, 80%, 85%, 90%, or 95%.

35. The method of any one of claims 1 to 34, wherein the cutoff is such that the PRI above the cutoff is predictive of a decrease of one or more IBD depleted cell types with a sensitivity of at least about 20%, 25%, 30%, 35%, 40%, 45%, 50%, 55%, 60%, 65%, 70%, 75%, 80%, 85%, 90%, or 95%.

36. The method of any one of claims 1 to 35, wherein the cutoff is such that the PRI above the cutoff is predictive of an increase of one or more IBD enriched cell types with a positive rate of at least about 10%, 15%, 20%, 25% 30%, 35%, 40%, 45%, 50%, 55%, 60%, 65%, 70%, or 75%.

37. The method of any one of claims 1 to 36, wherein the cutoff is such that the PRI above the cutoff is predictive of a decrease of one or more IBD depleted cell types with a positive rate of at least about 10%, 15%, 20%, 25%, 30%, 35%, 40%, 45%, 50%, 55%, 60%, 65%, 70%, or 75%.

38. The method of any one of claims 1 to 37, wherein the cutoff is such that the PRI above the cutoff is predictive of an increase of one or more IBD enriched cell types with an accuracy of at least about 50%, 55%, 60%, 65%, 70%, 75%, 80%, 85%, 90%, or 95%.

39. The method of any one of claims 1 to 38, wherein the cutoff is such that the PRI above the cutoff is predictive of a decrease of one or more IBD depleted cell types with an accuracy of at least about 50%, 55%, 60%, 65%, 70%, 75%, 80%, 85%, 90%, or 95%.

40. The method of any one of claims 28 to 39, wherein the one or more IBD enriched cell types comprises 1, 2, 3, 4, 5, 6, 7, 8, 9, 10, 11, 12, 13, or 14 cell types selected from the group consisting of activated fibroblasts, monocyte-derived dendritic cells (moDCs), and CD36+ endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts.

41. The method of any one of claims 29 to 40, wherein the one or more IBD depleted cell types comprises 1 or 2 cell types selected from the group consisting of Tuft cells and BEST4+ epithelial cells.

42. The method of any one of claims 1 to 41, wherein the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

43. The method of any one of claims 1 to 42, wherein the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

44. A method of treating an inflammatory, a fibrotic, or a fibrostenotic disease or condition in a subject, the method comprising: administering to the subject a therapeutically effective amount of an inhibitor of Tumor necrosis factor-like cytokine 1A (TL1A) activity or expression, wherein the subject is selected based on a comparison of a Predictive Response Index (PRI) to a cutoff according to (1) or (2):
(1) if the PRI has a positive correlation with a Response Probability Score (RPS), then the subject is selected if the PRI is above the cutoff; or
(2) if the PRI has a negative correlation with a RPS, then the subject is selected if the PRI is below the cutoff,
and wherein the PRI is calculated from a combination of polymorphisms determined from a sample from the subject and the comparison of the Predictive Response Index (PRI) to the cutoff according to (1) or (2) predicts a positive therapeutic response in the subject to a treatment with the inhibitor of TL1A activity or expression with a positive predictive value of at least about 29%.

45. A method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
(i) obtaining or having obtained a sample from the subject;
(ii) subjecting the sample to an assay adapted to detect a combination of polymorphisms;
(iii) calculating a Predictive Response Index (PRI) from the combination of polymorphisms, wherein the subject is determined to be suitable for treatment with an inhibitor of TL1A activity or expression based on a comparison of the PRI to a cutoff according to (1) or (2):
(1) if the PRI has a positive correlation with RPS, then the subject is determined to be suitable if the PRI is above the cutoff; or
(2) if the PRI has a negative correlation with RPS, then the subject is determined to be suitable if the PRI is below the cutoff, and
(b) treating the subject by administering a therapeutically effective amount of the inhibitor of TL1A activity or expression to the subject.

46. A method of determining a comparison of a Predictive Response Index (PRI) to a cutoff for a subject with an inflammatory, a fibrotic, or a fibrostenotic disease or condition, the method comprising:
(a) obtaining or having obtained a sample from the subject;
(b) subjecting the sample to an assay adapted to detect a combination of polymorphisms; and
(c) calculating the PRI from the combination of polymorphisms, wherein the comparison is determined according to (1) or (2):
(1) if the PRI has a positive correlation with RPS, then the subject is determined to be suitable for treatment with an inhibitor of TL1A activity or expression if the PRI is above the cutoff; or
(2) if the PRI has a negative correlation with RPS, then the subject is determined to be suitable for treatment with an inhibitor of TL1A activity or expression if the PRI is below the cutoff.

47. A method of selecting a subject with an inflammatory, a fibrotic, or a fibrostenotic disease or condition for treatment with an inhibitor of TL1A activity or expression, the method comprising:
(a) obtaining or having obtained a sample from the subject;
(b) subjecting the sample to an assay adapted to detect a combination of polymorphisms;
(c) calculating a Predictive Response Index (PRI) from the combination of polymorphisms; and
(d) selecting the subject for treatment with the inhibitor of TL1A activity or expression based on a comparison of the PRI to a cutoff according to (1) or (2):
(1) if the PRI has a positive correlation with RPS, then selecting the subject if the PRI is above the cutoff; or
(2) if the PRI has a negative correlation with RPS, then selecting the subject if the PRI is below the cutoff.

48. The method of any one of claims 45 to 47, wherein the method further comprises preparing DNA from the sample.

49. A method of treating an inflammatory, a fibrotic, or a fibrostenotic disease or condition in a subject, the method comprising: administering to the subject a therapeutically effective amount of an inhibitor of TL1A activity or expression, based, at least partially, on a Predictive Response Index (PRI) calculated by applying one or more statistical algorithms to a combination of polymorphisms detected from a sample obtained from the subject and determining a comparison of the PRI to a cutoff to predicts a positive therapeutic response in the subject to a treatment with the inhibitor of TL1A activity or expression.

50. A method of treating an inflammatory, a fibrotic, or a fibrostenotic disease or condition in a subject, the method comprising: administering an inhibitor of TL1A activity or expression to the subject that is predicted to exhibit a positive therapeutic response to the inhibitor of TL1A activity or expression, as determined by a Predictive Response Index (PRI) that is calculated by:
(a) detecting a presence of a combination of polymorphisms in a sample from the subject;
(b) applying a statistical algorithm to the combination of polymorphisms detected in step (a) to generate the PRI; and
(c) determining a comparison of the PRI to a cutoff.

51. A method of treating an inflammatory, a fibrotic, or a fibrostenotic disease or condition in a subject, the method comprising:
(a) determining whether the subject with an inflammatory, a fibrotic, or a fibrostenotic disease or condition is suitable for treatment with an inhibitor of TL1A activity or expression by:
(i) obtaining or having obtained a sample from the subject;
(ii) subjecting the sample to an assay adapted to detect a combination of polymorphisms;
(iii) calculating a Predictive Response Index (PRI) from the combination of polymorphisms, wherein the PRI is further determined in a comparison to a cutoff; and
(b) treating the subject by administering a therapeutically effective amount of the inhibitor of TL1A activity or expression to the subject.

52. The method of any one of claims 50 to 51, wherein the method further comprises preparing DNA from the sample.

53. The method of any one of claims 49 to 52, wherein the comparison of PRI to a cutoff is determined according to (1) or (2):
(1) if the PRI has a positive correlation with RPS, then determining the PRI of the subject if the PRI is above the cutoff; or
(2) if the PRI has a negative correlation with RPS, then determining the PRI of the subject if the PRI is below the cutoff.

54. The method of any one of claims 44 to 48 and 53, wherein the correlation coefficient is Pearson correlation coefficient or Spearman correlation coefficient.

55. The method of any one of claims 44 to 48 and 53 to 54, wherein if the PRI has a positive correlation with RPS then the positive correlation coefficient is at least about 0.6, at least about 0.65, at least about 0.7, at least about 0.75, at least about 0.8, at least about 0.85, at least about 0.95, at least about 0.99, or 1, or wherein if the PRI has a negative correlation with RPS then the negative correlation coefficient is at most about −0.6, at most about −0.65, at most about −0.7, at most about −0.75, at most about −0.8, at most about −0.85, at most about −0.95, at most about −0.99, or −1.

56. The method of any one of claims 44 to 48 and 53 to 55, wherein the RPS ranges from 0 to 1.

57. The method of claims 44 to 48 and 53 to 56, wherein if the PRI has a positive correlation with RPS then the cutoff is 0.5, or wherein if the PRI has a negative correlation with RPS then the cutoff is −0.5.

58. The method of any one of claims 44 to 48 and 53 to 57, wherein the RPS is calculated as 1/(1+e^{(-MRS)}), wherein the MRS is calculated as
$${β}_{0} + {\sum }_{i = 1}^{n} ( {β}_{i} ) \times ( {χ}_{i} ) ,$$
and wherein χᵢ is the mathematical representation of the ith single nucleotide polymorphisms (SNP) in the model and βᵢ is the weight for the ith SNP in the model.

59. The method of claim 58, wherein the SNP in the model is mathematically represented by χᵢ as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and 1 for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

60. The method of any one of claims 44 to 59, wherein the combination of polymorphisms comprises one or more polymorphisms selected from Table 27, or a proxy polymorphism in linkage disequilibrium therewith as determined with an R² of at least 0.85, or a combination thereof.

61. The method of any one of claims 44 to 48 and 53 to 60, wherein the cutoff is such that the comparison of the PRI to the cutoff according to (1) or (2) is predictive of a positive therapeutic response in the subject to a treatment with the inhibitor of TL1A activity or expression with a positive predictive value of at least about 29%, 30%, 35%, 40%, 45%, 50%, 51%, 55%, 60%, 65%, 70%, 75%, 80%, 85%, 90%, or 95%.

62. The method of any one of claims 44 to 48 and 53 to 61, wherein the cutoff is such that the comparison of the PRI to the cutoff according to (1) or (2) is predictive of a positive therapeutic response in the subject to a treatment with the inhibitor of TL1A activity or expression with a specificity of at least about 50%, 55%, 60%, 65%, 70%, 75%, 80%, 85%, 90%, or 95%.

63. The method of any one of claims 44 to 48 and 53 to 62, wherein the cutoff is such that the comparison of the PRI to the cutoff according to (1) or (2) is predictive of a positive therapeutic response in the subject to a treatment with the inhibitor of TL1A activity or expression with a negative predictive value of at least about 50%, 55%, 60%, 65%, 70%, 75%, 80%, 85%, 90%, or 95%.

64. The method of any one of claims 44 to 48 and 53 to 63, wherein the cutoff is such that the comparison of the PRI to the cutoff according to (1) or (2) is predictive of a positive therapeutic response in the subject to a treatment with the inhibitor of TL1A activity or expression with a sensitivity of at least about 20%, 25%, 30%, 35%, 40%, 45%, 50%, 55%, 60%, 65%, 70%, 75%, 80%, 85%, 90%, or 95%.

65. The method of any one of claims 44 to 48 and 53 to 64, wherein the cutoff is such that the comparison of the PRI to the cutoff according to (1) or (2) is predictive of a positive therapeutic response in the subject to a treatment with the inhibitor of TL1A activity or expression with a positive rate of at least about 10%, 15%, 20%, 25%, 30%, 35%, 40%, 45%, 50%, 55%, 60%, 65%, 70%, or 75%.

66. The method of any one of claims 44 to 48 and 53 to 65, wherein the cutoff is such that the comparison of the PRI to the cutoff according to (1) or (2) is predictive of a positive therapeutic response in the subject to a treatment with the inhibitor of TL1A activity or expression with an accuracy of at least about 50%, 55%, 60%, 65%, 70%, 75%, 80%, 85%, 90%, or 95%.

67. The method of any one of claims 44 to 48 and 53 to 66, wherein the cutoff is such that the comparison of the PRI to the cutoff according to (1) or (2) is predictive of an increase of one or more IBD enriched cell types with a positive predictive value of at least about 29%, 30%, 35%, 40%, 45%, 50%, 51%, 55%, 60%, 65%, 70%, 75%, 80%, 85%, 90%, or 95%.

68. The method of any one of claims 44 to 48 and 53 to 67, wherein the cutoff is such that the comparison of the PRI to the cutoff according to (1) or (2) is predictive of a decrease of one or more IBD depleted cell types with a positive predictive value of at least about 29%, 30%, 35%, 40%, 45%, 50%, 51%, 55%, 60%, 65%, 70%, 75%, 80%, 85%, 90%, or 95%.

69. The method of any one of claims 44 to 48 and 53 to 68, wherein the cutoff is such that the comparison of the PRI to the cutoff according to (1) or (2) is predictive of an increase of one or more IBD enriched cell types with a specificity of at least about 50%, 55%, 60%, 65%, 70%, 75%, 80%, 85%, 90%, or 95%.

70. The method of any one of claims 44 to 48 and 53 to 69, wherein the cutoff is such that the comparison of the PRI to the cutoff according to (1) or (2) is predictive of a decrease of one or more IBD depleted cell types with a specificity of at least about 50%, 55%, 60%, 65%, 70%, 75%, 80%, 85%, 90%, or 95%.

71. The method of any one of claims 44 to 48 and 53 to 70, wherein the cutoff is such that the comparison of the PRI to the cutoff according to (1) or (2) is predictive of an increase of one or more IBD enriched cell types with a negative predictive value of at least about 50%, 55%, 60%, 65%, 70%, 75%, 80%, 85%, 90%, or 95%.

72. The method of any one of claims 44 to 48 and 53 to 71, wherein the cutoff is such that the comparison of the PRI to the cutoff according to (1) or (2) is predictive of a decrease of one or more IBD depleted cell types with a negative predictive value of at least about 50%, 55%, 60%, 65%, 70%, 75%, 80%, 85%, 90%, or 95%.

73. The method of any one of claims 44 to 48 and 53 to 72, wherein the cutoff is such that the comparison of the PRI to the cutoff according to (1) or (2) is predictive of an increase of one or more IBD enriched cell types with a sensitivity of at least about 20%, 25%, 30%, 35%, 40%, 45%, 50%, 55%, 60%, 65%, 70%, 75%, 80%, 85%, 90%, or 95%.

74. The method of any one of claims 44 to 48 and 53 to 73, wherein the cutoff is such that the comparison of the PRI to the cutoff according to (1) or (2) is predictive of a decrease of one or more IBD depleted cell types with a sensitivity of at least about 20%, 25%, 30%, 35%, 40%, 45%, 50%, 55%, 60%, 65%, 70%, 75%, 80%, 85%, 90%, or 95%.

75. The method of any one of claims 44 to 48 and 53 to 74, wherein the cutoff is such that the comparison of the PRI to the cutoff according to (1) or (2) is predictive of an increase of one or more IBD enriched cell types with a positive rate of at least about 10%, 15%, 20%, 25%, 30%, 35%, 40%, 45%, 50%, 55% 60%, 65%, 70%, or 75%.

76. The method of any one of claims 44 to 48 and 53 to 75, wherein the cutoff is such that the comparison of the PRI to the cutoff according to (1) or (2) is predictive of a decrease of one or more IBD depleted cell types with a positive rate of at least about 10%, 15%, 20%, 25%, 30%, 35%, 40%, 45%, 50%, 55%, 60%, 65%, 70%, or 75%.

77. The method of any one of claims 44 to 48 and 53 to 76, wherein the cutoff is such that the comparison of the PRI to the cutoff according to (1) or (2) is predictive of an increase of one or more IBD enriched cell types with an accuracy of at least about 50%, 55%, 60%, 65%, 70%, 75%, 80%, 85%, 90%, or 95%.

78. The method of any one of claims 44 to 48 and 53 to 77, wherein the cutoff is such that the comparison of the PRI to the cutoff according to (1) or (2) is predictive of a decrease of one or more IBD depleted cell types with an accuracy of at least about 50%, 55%, 60%, 65%, 70%, 75%, 80%, 85%, 90%, or 95%.

79. The method of any one of claims 67 to 78, wherein the one or more IBD enriched cell types comprises 1, 2, 3, 4, 5, 6, 7, 8, 9, 10, 11, 12, 13, or 14 cell types selected from the group consisting of activated fibroblasts, monocyte-derived dendritic cells (moDCs), and CD36+endothelial cells, enterocytes and clonocytes, EECs, goblet cells, IgG plasma cells, Paneth cells, resident macrophages, TA cells, highly activated T cells, lymphatic epithelial cells, microfold cells, and myofibroblasts.

80. The method of any one of claims 68 to 79, wherein the one or more IBD depleted cell types comprises 1 or 2 cell types selected from the group consisting of Tuft cells and BEST4+ epithelial cells.

81. The method of any one of claims 44 to 80, wherein the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

82. The method of any one of claims 44 to 81, wherein the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

83. The method of any one of claims 1 to 82, wherein
(i) the PRI is calculated with a combination of polymorphisms selected from the combinations listed in column 2 of Table 31 and corresponding β coefficients listed in column 1 of Table 31;
(ii) the MRS is calculated with a combination of polymorphisms selected from the combinations listed in column 2 of Table 31 and corresponding β coefficients listed in column 1 of Table 31; and/or
(iii) the RPS is calculated with a combination of polymorphisms selected from the combinations listed in column 2 of Table 31 and corresponding β coefficients listed in column 1 of Table 31.

84. The method of any one of claims 1 to 83, wherein the combination of polymorphisms is detected in the sample by subjecting the sample to an assay configured to detect a presence of at least three nucleotides corresponding to nucleic acid position 501 within at least three of SEQ ID NOS: 2001-2048 and 2057-2059.

85. A computer-implemented system comprising at least one processor and instructions executable by the at least one processor to provide an application configured to determine a Response Probability Score (RPS) for a subject by performing operations comprising:
(a) receiving genotype data obtained from a sample from the subject with an inflammatory, a fibrotic, or a fibrostenotic disease or condition, wherein the genotype data comprises a combination of polymorphisms;
(b) applying a first statistical algorithm to the genotype data, the first statistical algorithm configured to produce a Model Risk Score (MRS) for the subject by performing operations comprising:
(i) assigning a weighted numerical value to each polymorphism in the combination of polymorphisms to produce a plurality of weighted values; and
(ii) summing the plurality of weighted values;
(c) applying a second statistical algorithm to the MRS, the second statistical algorithm configured to perform a logarithmic function on the MRS to produce a Response Probability Score (RPS); and
(d) applying a cutoff to the RPS, wherein the RPS relative to the cutoff is indicative that the subject is suitable for treatment with an inhibitor of TL1A activity or expression for treatment of the inflammatory, fibrotic, or fibrostenotic disease or condition

86. A computer-implemented system comprising at least one processor and instructions executable by the at least one processor to provide an application configured to determine a Response Probability Score (RPS) for a subject by performing operations comprising:
(a) receiving a plurality of multi-single nucleotide polymorphism (multi-SNP) models, wherein each multi-SNP model is predictive of a positive therapeutic response to an inhibitor of TL1A activity or expression for treatment of an inflammatory, a fibrotic, or a fibrostenotic disease or condition in the subject;
(b) receiving genotype data for a plurality of polymorphisms obtained from a sample from the subject;
(c) calculating a Model Risk Score (MRS) utilizing one or more statistical algorithms configured to perform operations comprising: (i) assigning a weighted numerical value to each polymorphism of the plurality of polymorphisms to produce a plurality of weighted values, and (ii) summing the plurality of weighted values; and
(d) applying a logarithmic scale and a cutoff to the MRS to produce a Response Probability Score (RPS).

87. The computer-implemented system of any one of claims 85 to 86, wherein the RPS ranges from 0 to 1.

88. The computer-implemented system of any one of claims 85 to 87, wherein the cutoff is 0.5.

89. The computer-implemented system of claim 85, wherein the genotype data is a combination of single nucleotide polymorphisms (SNPs).

90. The computer-implemented system of any one of claims 85 to 89, wherein the RPS is calculated as 1/(1+e^{(-MRS)}), wherein the MRS is calculated as
$${β}_{0} + {\sum }_{i = 1}^{n} ( {β}_{i} ) \times ( {χ}_{i} ) ,$$
and wherein χᵢ is the mathematical representation of the ith single nucleotide polymorphisms (SNP) in the model and βᵢ is the weight for the ith SNP in the model.

91. The computer-implemented system of any one of claims 85 to 90, wherein the MRS is calculated as
$${β}_{0} + {\sum }_{i = 1}^{n} ( {β}_{i} ) \times ( {χ}_{i} ) ,$$
and wherein χᵢ is the mathematical representation of the ith SNP in the model.

92. The computer-implemented system of any one of claims 86 to 91, wherein the SNP in the model is mathematically represented by y as:
(i) 0 for homozygous reference alleles, 1 for heterozygous reference and alternative alleles, and 2 for homozygous alternative alleles;
(ii) 1 for homozygous reference alleles, 1 for heterozygous reference and alternative alleles, and 0 for homozygous alternative alleles;
(iii) 0 for homozygous reference alleles, 1 for heterozygous reference and alternative alleles, and 1 for homozygous alternative alleles;
(iv) 0 for homozygous reference alleles, 0 for heterozygous reference and alternative alleles, and 1 for homozygous alternative alleles;
(v) 1 for homozygous reference alleles, 0 for heterozygous reference and alternative alleles, and 0 for homozygous alternative alleles; and/or
(vi) 0 for homozygous reference alleles, 1 for heterozygous reference and alternative alleles, and 0 for homozygous alternative alleles.

93. The computer-implemented system of any one of claims 86 to 92, wherein the combination of polymorphisms comprises one or more polymorphisms selected from Table 27, or a proxy polymorphism in linkage disequilibrium therewith as determined with an R² of at least 0.85, or a combination thereof.

94. The computer-implemented system of any one of claims 86 to 93, wherein the combination of polymorphisms comprise at least two, at least three, at least four, at least five, at least six, at least seven, at least eight, at least nine, at least ten, at least eleven, at least twelve, at least thirteen, at least fourteen, at least fifteen, or at least sixteen polymorphisms.

95. The computer-implemented system of any one of claims 86 to 94, wherein the PRI is calculated from a 1-SNP model selected from the 1-SNP models of Table 5, a 2-SNP combination selected from the 2-SNP models of Table 5, a 3-SNP combination selected from the 3-SNP models of Table 5, a 4-SNP combination selected from the 4-SNP models of Table 5, a 5-SNP combination selected from the 5-SNP models of Table 5, a 6-SNP combination selected from the 6-SNP models of Table 5, a 7-SNP combination selected from the 7-SNP models of Table 5, or a 8-SNP combination selected from the 8-SNP models of Table 5.

96. The computer-implemented system of any one of claims 86 to 95, wherein
(i) the MRS is calculated with a combination of polymorphisms selected from the combinations listed in column 2 of Table 31 and corresponding β coefficients listed in column 1 of Table 31; and/or
(ii) the RPS is calculated with a combination of polymorphisms selected from the combinations listed in column 2 of Table 31 and corresponding β coefficients listed in column 1 of Table 31.

97. The computer-implemented system of any one of claims 86 to 96, wherein the combination of polymorphisms is detected in the sample by subjecting the sample to an assay configured to detect a presence of at least three nucleotides corresponding to nucleic acid position 501 within at least three of SEQ ID NOS: 2001-2048 and 2057-2059.

98. The method of any one of claims 1 to 84 or the computer-implemented system of any one of claims 85 to 97, wherein the subject has been treated with an advanced IBD therapy prior to the treatment with the inhibitor of TL1A activity or expression.

99. The method of any one of claims 1 to 84 or the computer-implemented system of any one of claims 85 to 97, wherein the subject has not been treated with an advanced IBD therapy prior to the treatment with the inhibitor of TL1A activity or expression.

100. The method or the system of claim 98 or 99, wherein the advanced IBD therapy comprises one or more selected from the group consisting of a biologic therapeutic agent for IBD, an S1P1 modulator, or a JAK inhibitor.

101. The method or the system of claim 100, wherein the biologic therapeutic agent for IBD comprises an anti-TNFα antibody, an anti-IL23 antibody, or an anti-integrin a4P7 antibody.

102. The method of any one of claims 1 to 84 and 98 to 101 or the computer-implemented system of any one of claims 85 to 101, wherein the inhibitor of TL1A activity or expression is an antibody or antigen binding fragment thereof that binds to TL1A (anti-TL1A antibody or antigen binding fragment), wherein the anti-TL1A antibody or antigen binding fragment comprises a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and a light chain variable region comprising: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15.

103. The method of any one of claims 1 to 84 and 98 to 102 or the computer-implemented system of any one of claims 85 to 102, wherein the inhibitor of TL1A activity or expression is an anti-TL1A antibody or antigen binding fragment, wherein the anti-TL1A antibody or antigen binding fragment comprises a heavy chain variable domain comprising an amino acid sequence at least about 90% identical to any one of SEQ ID NOS: 101-135, or 310-302, and a light chain variable domain comprising an amino acid sequence at least about 90% identical to any one of SEQ ID NOS: 201-206 or 303.

104. The method or system of claim 102 or 103, wherein the heavy chain variable domain comprises an amino acid sequence at least about 91%, 92%, 93%, 94%, 95%, 96%, 97%, 98%, 99%, or 100% identical to any one of SEQ ID NOS: 101-135, or 310-302.

105. The method or system of any one of claims 102 to 104, wherein the light chain variable domain comprises an amino acid sequence at least about 91%, 92%, 93%, 94%, 95%, 96%, 97%, 98%, 99%, or 100% identical to any one of SEQ ID NOS: 201-206 or 303.

106. The method of any one of claims 1 to 84 and 98 to 105 or the computer-implemented system of any one of claims 85 to 105, wherein the inhibitor of TL1A activity or expression is an anti-TL1A antibody or antigen binding fragment, wherein the anti-TL1A antibody or antigen binding fragment comprises: (a) a heavy chain variable framework region comprising a human IGHV1-46*02 framework or a modified human IGHV1-46*02 framework; and (b) a light chain variable framework region comprising a human IGKV3-20 framework or a modified human IGKV3-20 framework; wherein the heavy chain variable framework region and the light chain variable framework region collectively comprise less than about 14 amino acid modifications from the human IGHV1-46*02 framework and the human IGKV3-20 framework.

107. The method or system of claim 106, wherein an amino acid modification of the less than 14 amino acid modifications comprises: (a) the amino acid modification is at position 47 in the heavy chain variable region, and the amino acid at position 47 is R, N, D, C, Q, E, G, H, I, L, K, M, F, P, S, T, W, Y, or V; (b) the amino acid modification is at position 45 in the heavy chain variable region, and the amino acid at position 45 is A, N, D, C, Q, E, G, H, I, L, K, M, F, P, S, T, W, Y, or V; (c) the amino acid modification is at position 55 in the heavy chain variable region, and the amino acid at position 55 is A, R, N, D, C, Q, E, G, H, I, L, K, F, P, S, T, W, Y, or V; (d) the amino acid modification is at position 78 in the heavy chain variable region, and the amino acid at position 78 is A, R, N, D, C, Q, E, G, H, I, L, K, M, F, P, S, T, W, or Y; (e) the amino acid modification is at position 80 in the heavy chain variable region, and the amino acid at position 80 is A, R, N, D, C, Q, E, G, H, I, L, K, F, P, S, T, W, Y, or V; (f) the amino acid modification is at position 82 in the heavy chain variable region, and the amino acid at position 82 is A, N, D, C, Q, E, G, H, I, L, K, M, F, P, S, T, W, Y, or V; (g) the amino acid modification is at position 89 in the heavy chain variable region, and the amino acid at position 89 is A, R, N, D, C, Q, E, G, H, I, L, K, M, F, P, S, T, W, or Y; or (h) the amino acid modification is at position 91 in the heavy chain variable region, and the amino acid at position 91 is A, R, N, D, C, Q, E, G, H, I, L, K, F, P, S, T, W, Y, or V; or a combination of two or more modifications selected from (a) to (h).

108. The method or system of claim 107, wherein an amino acid modification of the less than 14 amino acid modifications comprises: A47R, R45K, M55I, V78A, M80I, R82T, V89A, M91L in the heavy chain variable region, per Aho or Kabat numbering.

109. The method or system of claim 107, wherein an amino acid modification of the less than 14 amino acid modifications comprises: (a) a modification at amino acid position 54 in the light chain variable region; and/or (b) a modification at amino acid position 55 in the light chain variable region; per Aho or Kabat numbering.

110. The method or system of claim 106, wherein an amino acid modification of the less than 14 amino acid modifications comprises: (a) the amino acid modification is at position 54 of the light chain variable region, and the amino acid at position 54 is A, R, N, D, C, Q, E, G, H, I, K, M, F, P, S, T, W, Y, or V; and/or (b) the amino acid modification is at position 55 of the light chain variable region, and the amino acid at position 55 is A, R, N, D, C, Q, E, G, H, I, K, M, F, P, S, T, W, Y, or V.

111. The method or system of claim 110, wherein an amino acid modification of the less than 14 amino acid modifications comprises L54P and/or L55 W in the light chain variable region, per Aho or Kabat numbering.

112. The method of any one of claims 1 to 84 and 98 to 101 or the computer-implemented system of any one of claims 85 to 101, wherein the inhibitor of TL1A activity or expression is an antibody or antigen binding fragment thereof that binds to TL1A and comprises:
a heavy chain variable region comprising SEQ ID NO: 301 X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS, and
a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V.

113. The method of any one of claims 1 to 84 and 98 to 101 or the computer-implemented system of any one of claims 85 to 101, wherein the inhibitor of TL1A activity or expression is an antibody or antigen binding fragment thereof that binds to TL1A and comprises:
a heavy chain variable region comprising SEQ ID NO: 302 X1VQLVQSGAEVKKPGASVKVSCKAS[HCDR1]WVX2QX3PGQGLEWX4G[HCDR2]RX5 TX6TX7DTSTSTX8YX9ELSSLRSEDTAVYYC[HCDR3]WGQGTTVTVSS, and
a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V.

114. The method or system of any one of claims 112 to 113, wherein:
(A) X1 IS Q OR E,
(B) X2 IS R OR K
(C) X3 IS A OR R;
(D) X4 IS M OR I;
(E) X5 IS V OR A;
(F) X6 IS M OR I;
(G) X7 IS R OR T;
(H) X8 IS V OR A;
(I) X9 IS M OR L
(J) X10 IS L OR P;
(K) X11 IS L OR W; OR
(L) X1-X11 ARE ANY COMBINATION OF (A) TO (K).

115. The method or system of any one of claims 112 to 114, wherein the antibody or antigen binding fragment comprises a heavy chain CDR1 as set forth by SEQ ID NO: 1, a heavy chain CDR2 as set forth by any one of SEQ ID NOS: 2-5, a heavy chain CDR3 as set forth by any one of SEQ ID NOS: 6-9, a light chain CDR1 as set forth by SEQ ID NO: 10, a light chain CDR2 as set forth by SEQ ID NO: 11, and a light chain CDR3 as set forth by any one of SEQ ID NOS: 12-15.

116. The method or system of any one of claims 112 to 114, wherein the antibody or antigen binding fragment comprises a heavy chain framework (FR) 1 as set forth by SEQ ID NO: 304, a heavy chain FR2 as set forth by SEQ ID NO: 305 or SEQ ID NO: 313, a heavy chain FR3 as set forth by any one of SEQ ID NOS: 306, 307, 314, or 315, a heavy chain FR4 as set forth by SEQ ID NO: 308, a light chain FR1 as set forth by SEQ ID NO: 309, a light chain FR2 as set forth by SEQ ID NO: 310, a light chain FR3 as set forth by SEQ ID NO: 311, or a light chain FR4 as set forth by SEQ ID NO: 312, or a combination thereof.

117. The method of any one of claims 1 to 84 and 98 to 116 or the computer-implemented system of any one of claims 85 to 116, wherein the antibody or antigen binding fragment comprises a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of two or more selected from (a)-(uu), per Kabat numbering.

118. The method of any one of claims 1 to 116 or the computer-implemented system of any one of claims 85 to 116, wherein the antibody or antigen binding fragment comprises a human IgG4 Fc region.

119. The method of any one of claims 1 to 84 and 98 to 116 or the computer-implemented system of any one of claims 85 to 116, wherein the antibody or antigen binding fragment comprises a Fc region comprising a sequence at least about 90%, 91%, 92%, 93%, 94%, 95%, 96%, 97%, 98%, 99%, or 100% identical to any one of SEQ ID NOS: 320-362.

120. The method of any one of claims 1 to 84 and 98 to 116 or the computer-implemented system of any one of claims 85 to 116, wherein the antibody of antigen binding fragment comprises a fragment crystallizable (Fc) region comprising reduced antibody-dependent cell-mediated cytotoxicity (ADCC) function as compared to human IgG1 and/or reduced complement-dependent cytotoxicity (CDC) as compared to human IgG1.

121. The method of any one of claims 1 to 84 and 98 to 116 or the computer-implemented system of any one of claims 85 to 116, wherein the antibody or antigen binding fragment comprises a Fc region and wherein the Fc comprises the human IgG1 comprises SEQ ID NO: 320.

122. The method of any one of claims 1 to 84 and 98 to 116 or the computer-implemented system of any one of claims 85 to 116, wherein the antibody or antigen binding fragment comprises a Fc region and wherein the ADCC function of the Fc region comprising reduced ADCC is at least about 50% reduced as compared to human IgG1.

123. The method of any one of claims 1 to 84 and 98 to 116 or the computer-implemented system of any one of claims 85 to 116, wherein the antibody or antigen binding fragment comprises a Fc region and wherein the CDC function of the Fc region comprising reduced CDC is at least about 50% reduced as compared to human IgG1.

124. The method of any one of claims 1 to 84 and 98 to 116 or the computer-implemented system of any one of claims 85 to 116, wherein the antibody or antigen binding fragment comprises a Fc region and wherein the Fc comprises (i) a human IgG4 Fc region or (ii) a human IgG4 Fc region comprising (a) S228P, (b) S228P and L235E, or (c) S228P, F234A, and L235A, per Kabat numbering.

125. The method of any one of claims 1 to 84 and 98 to 116 or the computer-implemented system of any one of claims 85 to 116, wherein the antibody or antigen binding fragment comprises a Fc region and wherein the Fc comprises a human IgG2 Fc region; IgG2-IgG4 cross-subclass Fc region; IgG2-IgG3 cross-subclass Fc region; IgG2 comprising H268Q, V309L, A330S, P331S (IgG2m4), or IgG2 comprising V234A, G237A, P238S, H268A, V309L, A330S, P331S (IgG2σ).

126. The method of any one of claims 1 to 84 and 98 to 116 or the computer-implemented system of any one of claims 85 to 116, wherein the antibody or antigen binding fragment comprises a Fc region and wherein the Fc comprises a human IgG1 with a substitution selected from 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering.

127. The method of any one of claims 1 to 84 and 98 to 116 or the computer-implemented system of any one of claims 85 to 116, wherein the antibody or antigen binding fragment comprises a Fc region and wherein the Fc comprises a sequence at least about 90%, 91%, 92%, 93%, 94%, 95%, 96%, 97%, 98%, 99%, or 100% identical to any one of SEQ ID NOS: 320-362.

128. The method of any one of claims 1 to 84 and 98 to 116 or the computer-implemented system of any one of claims 85 to 116, wherein the antibody or antigen binding fragment comprises a Fc region and wherein the Fc comprises any one of SEQ ID NOs: 401-413 or a sequence at least about 90%, 91%, 92%, 93%, 94%, 95%, 96%, 97%, 98%, or 99% identical to any one of SEQ ID NOs: 401-413.

129. The method of any one of claims 1 to 84 and 98 to 116 or the computer-implemented system of any one of claims 85 to 116, wherein the antibody or antigen binding fragment comprises a heavy chain comprising any one of SEQ ID NOs: 501-513 or a sequence at least about 90%, 91%, 92%, 93%, 94%, 95%, 96%, 97%, 98%, or 99% identical to any one of SEQ ID NOs: 501-513.

130. The method of any one of claims 1 to 84 and 98 to 116 or the computer-implemented system of any one of claims 85 to 116, wherein the antibody or antigen binding fragment comprises a light chain comprising any one of SEQ ID NO: 514 or a sequence at least about 90%, 91%, 92%, 93%, 94%, 95%, 96%, 97%, 98%, or 99% identical to any one of SEQ ID NO: 514.

131. The method of any one of claims 1 to 84 and 98 to 130 or the computer-implemented system of any one of claims 85 to 130, wherein the combination of polymorphisms are detected in the sample by subjecting the sample to an assay configured to detect a presence of combination of nucleotides corresponding to nucleic acid position 501 within a combination of sequences selected from SEQ ID NOS: 2001-2041, and 2057-2059.

132. The method of any one of claims 1 to 84 and 98 to 131 or the computer-implemented system of any one of claims 85 to 131, wherein the inflammatory, fibrotic, or fibrostenotic disease or condition comprises inflammatory bowel disease, Crohn's disease, obstructive Crohn's disease, ulcerative colitis, intestinal fibrosis, intestinal fibrostenosis, rheumatoid arthritis, or primary sclerosing cholangitis.

133. The method or system of claim 132, wherein the Crohn's disease is ileal, ileocolonic, or colonic Crohn's disease.

134. The method of any one of claims 1 to 84 and 98 to 133 or the computer-implemented system of any one of claims 85 to 133, wherein the subject has, or is at risk for developing, a non-response or loss-of-response to a standard therapy comprising glucocorticosteroids, anti-TNF therapy, anti-a4-b7 therapy, anti-IL12p40 therapy, or a combination thereof.

135. The method of any one of claims 1 to 84 and 98 to 134 or the computer-implemented system of any one of claims 85 to 134, further comprising determining whether the subject with an inflammatory, a fibrotic, or a fibrostenotic disease or condition is suitable for treatment with an inhibitor of TL1A activity or expression based, at least in part, on the at least three polymorphisms detected in the sample.

136. The method or system of claim 135, wherein the at least three polymorphisms are detected by utilizing assay comprising a quantitative polymerase chain reaction (qPCR), nucleic acid sequencing reaction, or a genotyping array.

137. The method of any one of claims 1 to 84 and 98 to 136 or the computer-implemented system of any one of claims 85 to 136, wherein the combination of polymorphisms comprises or consists of any combination of polymorphisms described in row x of column 2 of Table 31, wherein x is any number between 2 to 1374.

138. The method of any one of claims 1 to 84 and 98 to 136 or the computer-implemented system of any one of claims 85 to 136, wherein the combination of polymorphisms comprises or consists of any combination of polymorphisms described in row x of column 2 of Table 31, wherein x is any number between 2 to 1374, wherein the polymorphisms of the combination of polymorphisms have β coefficients described in the row x of column 1 of Table 31, and wherein the polymorphisms of the combination of polymorphisms are numerically encoded as described in the row x of column 2 of Table 1.

139. The method of any one of claims 1 to 84 and 98 to 138, the method further comprising providing the sample to determine PRI for claims 1 to 9, 12 to 84, and 98 to 138, MRS for claims 10 to 43, 58 to 84 and 98 to 138, or RPS for claims 6 to 49, 53 to 84, and 98 to 138.

140. The method of any one of claims 1 to 3, 5 to 46, 48 to 84 and 98 to 139, the method further comprising selecting the subject according to PRI for claims 1 to 3, 5 to 9, 12 to 84 and 98 to 138.

141. The method of claim 140, wherein the PRI is RPS or MRS.

142. The method of any one of claims 1 to 84 and 98 to 141, the method further comprising
contacting genetic materials in the sample with one or more nucleic acid primer pairs having forward and reverse primers capable of hybridizing to one or more target nucleic acid sequences, the one or more target nucleic acid sequences collectively comprising chromosome positions of the polymorphisms of row x of column 2 of Table 31, wherein x is any number between 2 to 1374,
amplifying the target nucleic acid sequences by polymerase chain reactions with the nucleic acid primer pairs of the contacting step,
inputting results from the amplifying step into a computer system, and
analyzing the results via the computer system to determine PRI for claims 1 to 9, 12 to 84, and 98 to 138, MRS for claims 10 to 43, 58 to 84 and 98 to 138, or RPS for claims 6 to 49, 53 to 84, and 98 to 138, wherein the computer system comprises a storage unit configured to store the parameters of row y of column 1 of Table 31, wherein the y is identical to the x in the contacting step.

143. The method of any one of claims 14, 19 to 43, 58 to 84, and 98 to 142, wherein (i) the β₀ is about 0.0077127943934849 or (ii) the β₀ is about 0.008.

144. The method of any one of claims 14, 19 to 43, 58 to 84, and 98 to 143, wherein (i) the cutoff for MRS is about 0.0322446725024791 or (ii) the cutoff for MRS is about 0.03.
